(19) United States
(12) Patent Application Publication
(10) Pub. No.: US 2026/0250268 A1
(43) Pub. Date: Aug. 27, 2026

(54) COMPOUNDS AS PRMT5 DEGRADERS AND USES THEREOF

(71) Applicant: Oncopia Therapeutics, Inc. d/b/a/ SK Life Science Labs, King of Prussia, PA (US)

(72) Inventors: Xuqing ZHANG, King of Prussia, PA (US); Guozhang XU, King of Prussia, PA (US); Qiaolin DENG, King of Prussia, PA (US); Wenxue WU, King of Prussia, PA (US); Jeremy ROACH, King of Prussia, PA (US)

(21) Appl. No.: 19/536,810

(22) Filed: Feb. 11, 2026

Related U.S. Application Data
(60) Provisional application No. 63/757,033, filed on Feb. 11, 2025.

(51) Int. Cl. C07D 403/04; C07D 401/04; C07D 487/04

(52) U.S. Cl. CPC C07D 403/04; C07D 401/04; C07D 487/04

(57) ABSTRACT

Described herein are compounds of Formula I and their pharmaceutically acceptable salts, solvates, and stereoisomers, as well as their uses (e.g., as PRMT5 degraders).

### RELATED APPLICATION

This application claims the benefit of and priority to U.S. Provisional Application No. 63/757,033, filed Feb. 11, 2025, the contents of which are incorporated herein by reference in their entireties.

### BACKGROUND

As the predominant member of type II protein arginine methyltransferases (PRMTs), protein arginine methyltransferase 5 (PRMT5) catalyzes monomethylation and symmetric dimethylation of arginine residues of its histone substrates, including H3R2, H3R8, and H4R3, and its nonhistone substrates, including p53, EGFR, N-MYC, SmD3, and RNA polymerase II, when interacting with its binding partner MEP50. Through methylation of these substrates, PRMT5 regulates multiple biological processes, such as chromatin remodeling, gene expression, mRNA splicing, DNA replication and repair, and cell cycle regulation.

The aberrant expression of PRMT5 has been associated with infectious disease, heart disease, and cancers, including breast cancer, lung cancer, and hepatocellular cancer (HCC). Genetic knockdown of PRMT5 was effective to suppress tumor growth in vitro and in vivo. For example, PRMT5 knockdown decreased the proliferation, invasion, and migration of HCC cell lines. The knockdown of PRMT5 in AR-positive LNCaP prostate cancer cells completely suppressed tumor growth in xenograft mouse models. In addition, the knockdown of PRMT5 drastically prolonged the survival in a murine model of BCR-ABL-driven chronic myelogenous leukemia and of MYC-driven B cell lymphoma. Moreover, the PRMT5 knockdown effectively suppressed the growth of patient-derived xenograft glioblastoma (GBM) tumors in vivo. Collectively, these findings suggest that PRMT5 down-regulation may provide a potential therapeutic strategy for treating cancer.

Recently, proteolysis targeting chimera (PROTAC) has emerged as a powerful technology to reduce the protein levels of the target of interest by hijacking the cellular ubiquitin-proteasome system. PROTACs are can phenocopy the effects of genetic knockdown or knockout as they not only inhibit the target proteins as traditional inhibitors do but also more importantly eliminate the other functions (e.g., scaffolding function) of the protein targets. Another potential advantage of PROTACs over occupancy-driven small-molecule inhibitors is that PROTACs may potentially overcome the resistance to small-molecule inhibitor treatments.

### SUMMARY

In some aspects, the present disclosure provides compounds of Formula I′:

T-L-V(I′),

or a pharmaceutically acceptable salt, solvate, or stereoisomer thereof, wherein:
T is of Formula I′-1

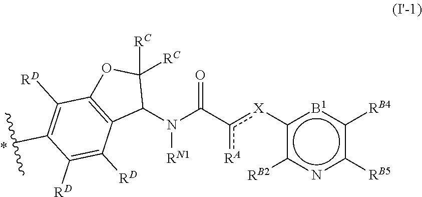

wherein:
* denotes attachment to L;
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
B¹ is N, CR^{B1}, or NR^{BN1}, as valency permits;
R^{B1}, R^{B2}, R^{B4}, and R^{B5} are independently hydrogen, halogen, —CN, —NO₂, —OH, —NH₂, —C(═O)NH₂, —C(═O)(C₁₋₆ alkylamino), C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, or C₁₋₆ alkyl; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, or 3- to 12-membered heterocyclyl; or
R^{BN1} and R^{B4}, together with the intervening atoms to which they are attached, form 5-membered heteroaryl optionally substituted with one or more halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, or 3- to 12-membered heterocyclyl;
R^{N1} is hydrogen, C₁₋₆ alkyl, or C₃₋₁₂ carbocyclyl, wherein the C₁₋₆ alkyl or C₃₋₁₂ carbocyclyl is optionally substituted with one or more halogen, —CN, —NO₂, —OH, —NH₂, or C₃₋₁₂ carbocyclyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
each R^{D} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
L is of Formula I′-2

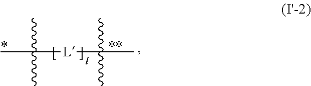

wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N(R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆-10 arylene, or 5- to 10-membered heteroarylene, wherein the —O—(C₁₋₆ alkylene)-, —(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆-10 arylene, or 5- to 10-membered heteroarylene is optionally substituted with one or more oxo, halogen, —CN, —NO₂, —OH, —NH₂, or C₁₋₆ alkyl;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(═O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
1 is an integer selected from 0 to 10;
V is of Formula I′-3

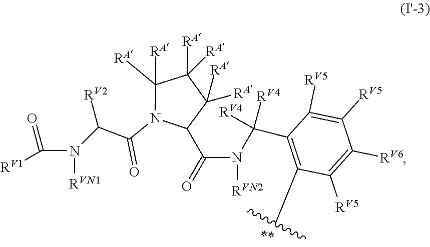

wherein:
* denotes attachment to L;
R^{V1} is hydrogen, C₁₋₆ alkyl, C₃₋₁₂ carbocyclyl, or 3- to 6-membered heterocyclyl, wherein the C₁₋₆ alkyl, C₃-6 carbocyclyl, or 3- to 6-membered heterocyclyl is optionally substituted with one or more halogen, —CN, —NO₂, —OH, —NH₂, or C₁₋₆ alkyl optionally substituted with one or more halogen;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen, C₁₋₆ alkyl, C₃₋₆ carbocyclyl, 3- to 6-membered heterocyclyl, C₆₋₁₀ aryl, or 5- to 10-membered heteroaryl, wherein the C₁₋₆ alkyl, C₃₋₆ carbocyclyl, 3- to 6-membered heterocyclyl, C₆₋₁₀ aryl, or 5- to 10-membered heteroaryl is optionally substituted with one or more halogen, —CN, —NO₂, —OH, —NH₂, or C₁₋₆ alkyl;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, —NH₂, C₃₋₁₂ carbocyclyl, 3- to 12-membered heterocyclyl, C₆₋₁₀ aryl, or 5- to 10-membered heteroaryl;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is hydrogen, halogen, —CN, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl optionally substituted with one or more-CN, C₂₋₆ alkenyl, or C₂₋₆ alkynyl.

In some aspects, the present disclosure provides conjugates comprising a compound disclosed herein, wherein the compound is attached to a conjugate partner.

In some aspects, the present disclosure provides pharmaceutical compositions comprising a compound disclosed herein, and a pharmaceutically acceptable excipient.

In some aspects, the present disclosure provides methods of degrading a PRMT5 protein in a subject, comprising administering to the subject a compound disclosed herein.

In some aspects, the present disclosure provides uses of a compound disclosed herein in the manufacture of a medicament for degrading a PRMT5 protein in a subject.

In some aspects, the present disclosure provides compounds disclosed herein for use in degrading a PRMT5 protein in a subject.

In some aspects, the present disclosure provides methods of reducing the amount of a PRMT5 protein in a subject (e.g., in a biological sample (e.g., a cell or a tissue) obtained from the subject), comprising administering to the subject a compound disclosed herein.

In some aspects, the present disclosure provides uses of a compound disclosed herein in the manufacture of a medicament for reducing the amount of a PRMT5 protein in a subject (e.g., in a biological sample (e.g., a cell or a tissue) obtained from the subject).

In some aspects, the present disclosure provides compounds disclosed herein for use in reducing the amount of a PRMT5 protein in a subject (e.g., in a biological sample (e.g., a cell or a tissue) obtained from the subject).

In some aspects, the present disclosure provides methods of treating or preventing a disease or disorder in a subject in need thereof, comprising administering to the subject a compound disclosed herein (e.g., in a therapeutically effective amount).

In some aspects, the present disclosure provides methods of treating a disease or disorder in a subject in need thereof, comprising administering to the subject a compound disclosed herein (e.g., in a therapeutically effective amount).

In some aspects, the present disclosure provides uses of a compound disclosed herein in the manufacture of a medicament for treating or preventing a disease or disorder in a subject in need thereof.

In some aspects, the present disclosure provides uses of a compound disclosed herein in the manufacture of a medicament for treating a disease or disorder in a subject in need thereof.

In some aspects, the present disclosure provides compounds disclosed herein for use in treating or preventing a disease or disorder in a subject in need thereof.

In some aspects, the present disclosure provides compounds disclosed herein for use in treating a disease or disorder in a subject in need thereof.

### DETAILED DESCRIPTION

The present disclosure relates to compounds and compositions that may be useful as PRMT5 protein degraders. The present disclosure also relates to methods of degrading a PRMT5 protein comprising contacting the PRMT5 protein with a compound or composition disclosed herein. The present disclosure also relates to methods of treating or preventing a PRMT5 protein-mediated disease or condition in a subject in need thereof by administering (e.g., in a therapeutically effective amount) a compound or composition disclosed herein.

### Compounds of the Application

In some aspects, the present disclosure provides compounds of Formula I′

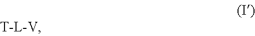

or a pharmaceutically acceptable salt, solvate, or stereoisomer thereof, wherein:
T is of Formula I′-1

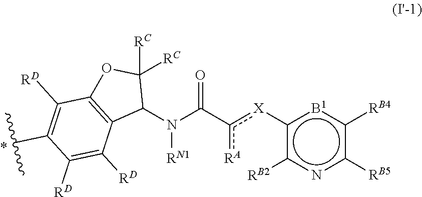

wherein:
attachment to L;
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
B¹ is N, CR^{B1}, or NR^{BN1}, as valency permits;
R^{B1}, R^{B2}, R^{B4}, and R^{B5} are independently hydrogen, halogen, —CN, —NO₂, —OH, —NH₂, —C(═O)NH₂, —C(═O)(C₁₋₆ alkylamino), C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, or C₁₋₆ alkyl; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, or 3- to 12-membered heterocyclyl; or
R^{BN1} and R^{B4}, together with the intervening atoms to which they are attached, form 5-membered heteroaryl optionally substituted with one or more halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, or 3- to 12-membered heterocyclyl;
R^{N1} is hydrogen, C₁₋₆ alkyl, or C₃₋₁₂ carbocyclyl, wherein the C₁₋₆ alkyl or C₃₋₁₂ carbocyclyl is optionally substituted with one or more halogen, —CN, —NO₂, —OH, —NH₂, or C₃₋₁₂ carbocyclyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
each R^{D} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
L is of Formula I′-2

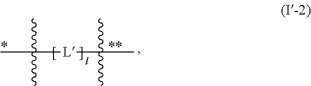

wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N(R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O—(C₁₋₆ alkylene)-, —(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more oxo, halogen, —CN, —NO₂, —OH, —NH₂, or C₁₋₆ alkyl;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(═O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
1 is an integer selected from 0 to 10;
V is of Formula I′-3

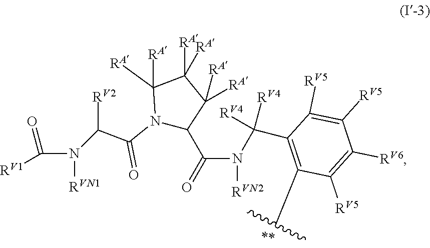

wherein:
* denotes attachment to L;
R^{V1} is hydrogen, C₁₋₆ alkyl, C₃₋₁₂ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, —NH₂, or C₁₋₆ alkyl optionally substituted with one or more halogen;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen, C₁₋₆ alkyl, C₃₋₆ carbocyclyl, 3- to 6-membered heterocyclyl, C₆₋₁₀ aryl, or 5- to 10-membered heteroaryl, wherein the C₁₋₆ alkyl, C₃₋₆ carbocyclyl, 3- to 6-membered heterocyclyl, C₆₋₁₀ aryl, or 5- to 10-membered heteroaryl is optionally substituted with one or more halogen, —CN, —NO₂, —OH, —NH₂, or C₁₋₆ alkyl;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, —NH₂, C₃₋₁₂ carbocyclyl, 3- to 12-membered heterocyclyl, C₆₋₁₀ aryl, or 5- to 10-membered heteroaryl;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is hydrogen, halogen, —CN, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl optionally substituted with one or more-CN, C₂₋₆ alkenyl, or C₂₋₆ alkynyl.

In some embodiments, the present disclosure provides compounds of Formula I

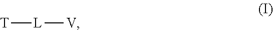

or a pharmaceutically acceptable salt, solvate, or stereoisomer thereof, wherein:
T is of Formula 1-1

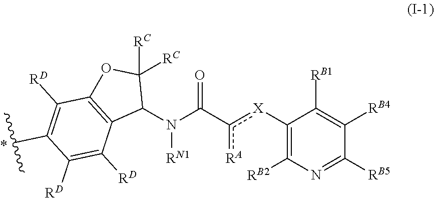

wherein:
* denotes attachment to L;
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
R^{B1}, R^{B2}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, or —NH₂; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl;
R^{N1} is hydrogen, C₁₋₆ alkyl, or C₃₋₁₂ carbocyclyl, wherein the C₁₋₆ alkyl or C₃₋₁₂ carbocyclyl is optionally substituted with one or more halogen, —CN, —NO₂, —OH, —NH₂, or C₃₋₁₂ carbocyclyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
each R^{D} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
L is of Formula 1-2

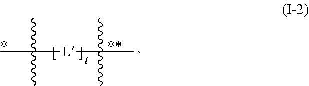

wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N (R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O—(C₁₋₆ alkylene)-, —(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more halogen, —CN, —NO₂, —OH, or —NH₂;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(—O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
1 is an integer selected from 0 to 10;
V is of Formula 1-3

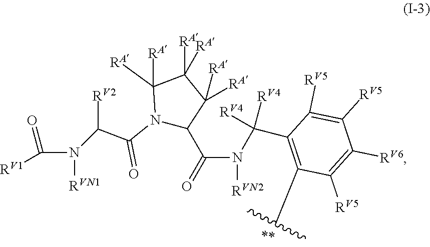

wherein:
* denotes attachment to L;
R^{V1} is hydrogen, C₁₋₆ alkyl, C₃₋₆ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, or —NH₂;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen or C₁₋₆ alkyl, wherein the C₁₋₆ alkyl is optionally substituted with one or more halogen, —CN, —NO₂, —OH, or —NH₂;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl, C₂₋₆ alkenyl, or C₂₋₆ alkynyl.

In some embodiments, the compound is of Formula (I′-a)

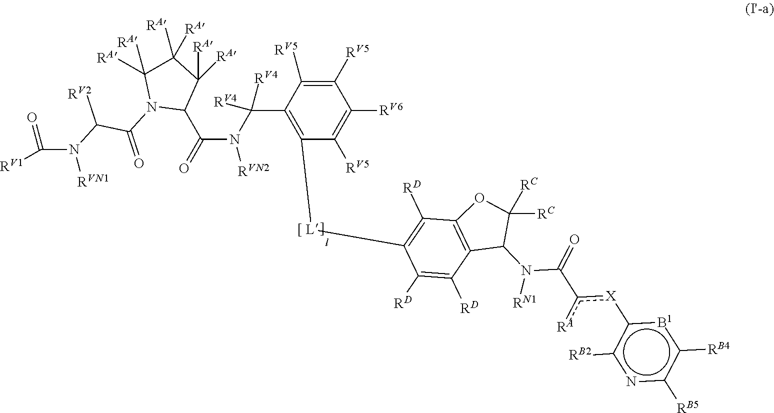

or a pharmaceutically acceptable salt, solvate, or stereoisomer thereof.

In some embodiments, the compound is of Formula (I′-a-i), (I′-a-ii), or (I′-a-iii)

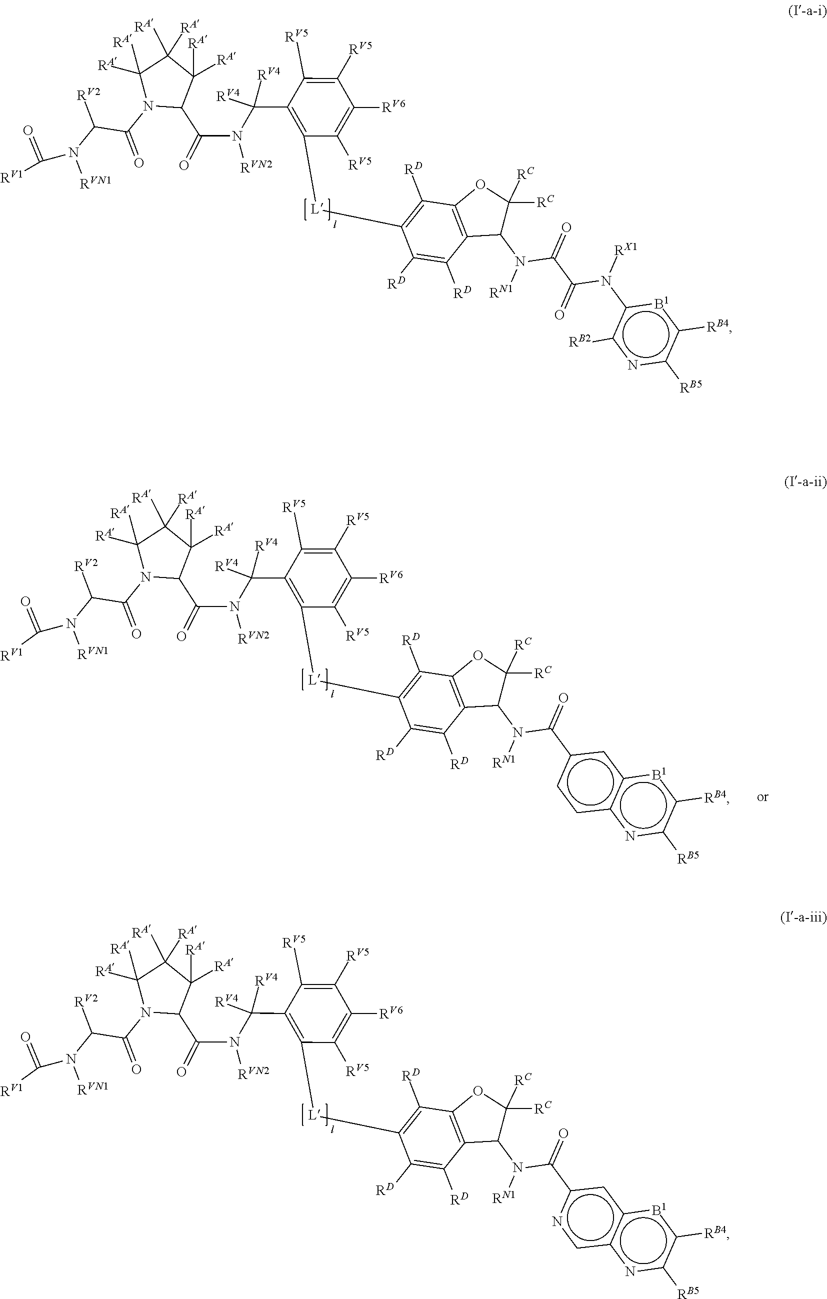

or a pharmaceutically acceptable salt, solvate, or stereoisomer thereof.

### Variable T

In some embodiments, at least one [symbol] is a single bond, as valency permits.

In some embodiments, at least one [symbol] is a double bond, as valency permits.

In some embodiments, one [symbol] is a single bond, as valency permits.

In some embodiments, one [symbol] is a double bond, as valency permits.

In some embodiments, X is N(R^{X1}), and R^{A} is oxo.

In some embodiments, X is C(R^{X2}), and R^{A} and R^{B2}, together with the intervening atoms to which they are attached, form C₆ aryl optionally substituted with one or more halogen (e.g., F, Cl, Br, or I), —CN, —NO₂, —OH, or —NH₂.

In some embodiments, X is C(R^{X2}), and R^{A} and R^{B2}, together with the intervening atoms to which they are attached, form 6-membered heteroaryl (e.g., heteroaryl comprising one 6-membered ring and 1-3 heteroatoms selected from N, O, and S) optionally substituted with one or more halogen (e.g., F, Cl, Br, or I), —CN, —NO₂, —OH, or —NH₂.

In some embodiments, X is C(R^{X2}), and R^{A} and R^{B2}, together with the intervening atoms to which they are attached, form pyridinyl.

In some embodiments, R^{X1} is hydrogen.

In some embodiments, R^{X1} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, R^{X2} is hydrogen.

In some embodiments, R^{X2} is halogen (e.g., F, Cl, Br, or I).

In some embodiments, R^{X2} is —CN.

In some embodiments, R^{X2} is —NO₂.

In some embodiments, R^{X2} is —OH.

In some embodiments, R^{X2} is —NH₂.

In some embodiments, R^{X2} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), wherein the C₁₋₆ alkyl is optionally substituted with one or more halogen (e.g., F, Cl, Br, or I), —CN, —NO₂, —OH, or —NH₂.

In some embodiments, B¹ is N.

In some embodiments, B¹ is CR^{B1}.

In some embodiments, B¹ is NR^{BN1}.

In some embodiments, R^{B1} is hydrogen.

In some embodiments, R^{B1} is halogen (e.g., F, Cl, Br, or I).

In some embodiments, R^{B1} is —CN.

In some embodiments, R^{B1} is —NO₂.

In some embodiments, R^{B1} is —OH.

In some embodiments, R^{B1} is —NH₂.

In some embodiments, R^{B1} is —C(═O)NH₂.

In some embodiments, R^{B1} is —C(═O)(C₁₋₆ alkylamino).

In some embodiments, R^{B1} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆).

In some embodiments, R^{B1} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B1} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B1} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B1} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B1} is C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₂), cycloheptadienyl(C₂), cycloheptatrienyl(C₇), cyclooctyl(C₈), cyclooctenyl(C₅), bicyclo[2.2.1]heptanyl(C₂), bicyclo[2.2.2]octanyl(C₅), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B1} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B1} is C₆₋₁₀ aryl (e.g., phenyl or naphthyl) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B1} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B2} is hydrogen.

In some embodiments, R^{B2} is halogen (e.g., F, Cl, Br, or I).

In some embodiments, R^{B2} is —CN.

In some embodiments, R^{B2} is —NO₂.

In some embodiments, R^{B2} is —OH.

In some embodiments, R^{B2} is —NH₂.

In some embodiments, R^{B2} is —C(═O)NH₂.

In some embodiments, R^{B2} is —C(═O)(C₁₋₆ alkylamino).

In some embodiments, R^{B2} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, R^{B2} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B2} is C₁₋₆ alkylamino (e.g., dimethylamino, diethylamino, di-n-propylamino, di-i-propylamino, di-n-butylamino, di-i-butylamino, di-s-butylamino, di-i-butylamino, dipentylamino, dihexylamino, methylethylamino, methyl-n-propylamino, methyl-i-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, f-butylhexylamino, or pentylhexylamino) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B2} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B2} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B2} is C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₂), cycloheptatrienyl(C₇), cyclooctyl(C₅), cyclooctenyl(C₄), bicyclo[2.2.1]heptanyl(C₇), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), S-butyl(C₄), t-butyl(C₄), pentyl(C₅), or hexyl(C₆)).

In some embodiments, R^{B2} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), S-butyl(C₄), t-butyl(C₄), pentyl(C₅), or hexyl(C₆)).

In some embodiments, R^{B2} is C₆₋₁₀ aryl (e.g., phenyl or naphthyl) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B2} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B4} is hydrogen.

In some embodiments, R^{B4} is halogen (e.g., F, Cl, Br, or I).

In some embodiments, R^{B4} is —CN.

In some embodiments, R^{B4} is —NO₂.

In some embodiments, R^{B4} is —OH.

In some embodiments, R^{B4} is —NH₂.

In some embodiments, R^{B4} is —C(═O)NH₂.

In some embodiments, when R^{B4} is —C(═O)NH₂, then R^{B5} is not C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-i-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino).

In some embodiments, when R^{B4} is —C(═O)NH₂, then RBS is not —NH(CH₃).

In some embodiments, when R^{B4} is —C(═O)NH₂, then R^{B5} is hydrogen.

In some embodiments, R^{B4} is —C(═O)(C₁₋₆ alkylamino).

In some embodiments, R^{B4} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, R^{B4} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B4} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B4} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B4} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B4} is C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₇), cycloheptatrienyl(C₇), cyclooctyl(C₄), cyclooctenyl(C₈), bicyclo[2.2.1]heptanyl(C₇), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), S-butyl(C₄), t-butyl(C₄), pentyl(C₅), or hexyl(C₆)).

In some embodiments, R^{B4} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), S-butyl(C₄), t-butyl(C₄), pentyl(C₅), or hexyl(C₆).

In some embodiments, R^{B4} is C₆₋₁₀ aryl (e.g., phenyl or naphthyl) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B4} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{B1} is hydrogen or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), and R^{B4} is halogen (e.g., F, Cl, Br, or I), —CN, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more-OH, C₂₋₆ alkynyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₂), cycloheptadienyl(C₇), cycloheptatrienyl(C₇), cyclooctyl(C₈), cyclooctenyl(C₉), bicyclo[2.2.1]heptanyl(C₂), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), or 3- to 12-membered heterocyclyl (e.g., heterocyclyl comprising one or two 3- to 8-membered rings and 1-5 heteroatoms selected from N, O, and S).

In some embodiments, R^{B5} is hydrogen.

In some embodiments, R^{B5} is halogen (e.g., F, Cl, Br, or I).

In some embodiments, R^{B5} is —CN.

In some embodiments, R^{B5} is —NO₂.

In some embodiments, R^{B5} is —OH.

In some embodiments, R^{B5} is —NH₂.

In some embodiments, R^{B5} is —C(═O)NH₂.

In some embodiments, R^{B5} is —C(═O)(C₁₋₆ alkylamino).

In some embodiments, R^{B5} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, R^{B5} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, R^{B5} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, R^{B5} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, or —NH₂.

In some embodiments, R^{B5} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, or —NH₂.

In some embodiments, R^{B5} is C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₂), cycloheptatrienyl(C₇), cyclooctyl(C₄), cyclooctenyl(C₅), bicyclo[2.2.1]heptanyl(C₇), bicyclo[2.2.2]octanyl(C₄), cyclononyl(C₉), cyclononenyl(C₉), cyclodecyl(C₁₀), cyclodecenyl (C₁₀), octahydro-1H-indenyl(C₅), decahydronaphthalenyl(C₁₀), or spiro[4.5]decanyl(C₁₀)) optionally substituted with one or more halogen (e.g., F, Cl, Br, or I), —CN, —NO₂, —OH, or —NH₂.

In some embodiments, R^{B5} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B5} is C₆₋₁₀ aryl (e.g., phenyl or naphthyl) optionally substituted with one or more halogen (e.g., F, Cl, Br, or I), —CN, —NO₂, —OH, or —NH₂.

In some embodiments, R^{B5} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, or —NH₂.

In some embodiments, R^{B1} and R^{B4}, together with the intervening atoms to which they are attached, form 3- to 8-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₁), cycloheptatrienyl(C₇), cyclooctyl(C₄), cyclooctenyl(C₅), bicyclo[2.2.1]heptanyl(C₇), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), or 3- to 12-membered heterocyclyl (e.g., heterocyclyl comprising one or two 3- to 8-membered rings and 1-5 heteroatoms selected from N, O, and S).

In some embodiments, R^{B1} and R^{B4}, together with the intervening atoms to which they are attached, form 5- to 6-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆), C₁₋₆ alkoxy (e.g., methoxy (C₁), ethoxy (C₂), propoxy (C₃), i-propoxy (C₃), n-butoxy (C₄), i-butoxy (CA), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-i-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₅), cycloheptyl(C₂), cycloheptenyl (C), cycloheptadienyl(C₂), cycloheptatrienyl(C₇), cyclooctyl(C₅), cyclooctenyl(C₈), bicyclo[2.2.1]heptanyl(C₇), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), or 3- to 12-membered heterocyclyl (e.g., heterocyclyl comprising one or two 3- to 8-membered rings and 1-5 heteroatoms selected from N, O, and S).

In some embodiments, R^{B1} and R^{B4}, together with the intervening atoms to which they are attached, form dihydrofuran or pyrazole optionally substituted with one or more halogen or C₁₋₆ alkyl.

In some embodiments, R^{BN1} and R^{B4}, together with the intervening atoms to which they are attached, form 5-membered heteroaryl (e.g., heteroaryl comprising one 5-membered ring and 1-3 heteroatoms selected from N, O, and S) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl (CA), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (CA), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl (CA), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₇), cycloheptatrienyl (C), cyclooctyl(C₈), cyclooctenyl(C₄), bicyclo[2.2.1]heptanyl(C₂), bicyclo[2.2.2]octanyl(C₅), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), or 3- to 12-membered heterocyclyl (e.g., heterocyclyl comprising one or two 3- to 8-membered rings and 1-5 heteroatoms selected from N, O, and S).

In some embodiments, R^{N1} is hydrogen.

In some embodiments, R^{N1} is C₁₋₆ alkyl optionally substituted with one or more halogen (e.g., F, Cl, Br, or I), —CN, —NO₂, —OH, —NH₂, or C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₂), cycloheptatrienyl(C₂), cyclooctyl(C₄), cyclooctenyl(C₄), bicyclo[2.2.1]heptanyl(C₂), bicyclo[2.2.2]octanyl(C₅), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)).

In some embodiments, R^{N1} is C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₂), cycloheptatrienyl(C₂), cyclooctyl(C₄), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)) optionally substituted with one or more halogen (e.g., F, Cl, Br, or I), —CN, —NO₂, —OH, —NH₂, or C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₂), cycloheptadienyl(C₂), cycloheptatrienyl(C₂), cyclooctyl(C₄), cyclooctenyl(C₅), bicyclo[2.2.1]heptanyl(C₇), bicyclo[2.2.2]octanyl(C₅), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)).

In some embodiments, at least one R^{C} is hydrogen.

In some embodiments, each R^{C} is hydrogen.

In some embodiments, at least one R^{C} is halogen (e.g., F, Cl, Br, or I).

In some embodiments, at least one R^{C} is —CN.

In some embodiments, at least one R^{C} is —NO₂.

In some embodiments, at least one R^{C} is —OH.

In some embodiments, at least one R^{C} is —NH².

In some embodiments, at least one R^{C} is C₁₋₆ alkyl (e.g., methyl(C₁), ethyl(C₂), n-propyl(C₃), i-propyl(C₃), n-butyl(C₄), i-butyl(C₄), s-butyl (CA), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, at least one R^{C} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{C} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-i-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{C} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, or —NH₂.

In some embodiments, at least one R^{C} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, or —NH₂.

In some embodiments, at least one R^{D} is hydrogen.

In some embodiments, at least two R^{D} are hydrogen.

In some embodiments, one R^{D} is hydrogen.

In some embodiments, two R^{D} are hydrogen.

In some embodiments, three R^{D} are hydrogen.

In some embodiments, at least one R^{D} is halogen (e.g., F, Cl, Br, or I).

In some embodiments, at least one R^{D} is —CN.

In some embodiments, at least one R^{D} is —NO₂.

In some embodiments, at least one R^{D} is —OH.

In some embodiments, at least one R^{D} is —NH₂.

In some embodiments, at least one R^{D} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, at least one R^{D} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{D} is C₁₋₆ alkylamino (e.g., dimethylamino, diethylamino, di-n-propylamino, di-i-propylamino, di-n-butylamino, di-i-butylamino, di-s-butylamino, di-i-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-¿-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{D} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, or —NH₂.

In some embodiments, at least one R^{D} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, or —NH₂.

In some embodiments, T is of Formula I-1.

In some embodiments, T is of Formula I′-1.

In some embodiments, T is of Formula I-1-a

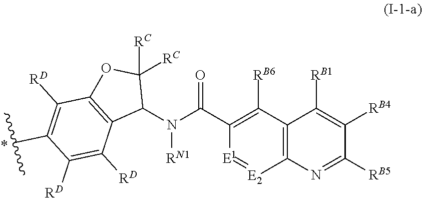

wherein:
E¹ is N or CR^{B7};
E² is N or CR^{B8}, and
R^{B6}, R^{B7}, and R^{B8} are independently hydrogen, halogen, —CN, —NO₂, —OH, or —NH₂.

In some embodiments, E¹ is N.

In some embodiments, E¹ is CR^{B7}.

In some embodiments, E² is N.

In some embodiments, E² is CR^{B8}

In some embodiments, E¹ is N, and E² is CR^{B8}.

In some embodiments, E¹ is CR^{B7}, and E² is N.

In some embodiments, R^{B6} is hydrogen.

In some embodiments, R^{B6} is halogen (e.g., F, Cl, Br, or I).

In some embodiments, R^{B6} is —CN.

In some embodiments, R^{B6} is —NO₂.

In some embodiments, R^{B6} is —OH.

In some embodiments, R^{B6} is —NH₂.

In some embodiments, R^{B7} is hydrogen.

In some embodiments, R^{B7} is halogen (e.g., F, Cl, Br, or I).

In some embodiments, R^{B7} is —CN.

In some embodiments, R^{B7} is —NO₂.

In some embodiments, R^{B7} is —OH.

In some embodiments, R^{B7} is —NH₂.

In some embodiments, R^{B8} is hydrogen.

In some embodiments, R^{B8} is halogen (e.g., F, Cl, Br, or I).

In some embodiments, R^{B8} is —CN.

In some embodiments, R^{B8} is —NO₂.

In some embodiments, R^{B8} is —OH.

In some embodiments, R^{B8} is —NH₂.

In some embodiments, T is of Formula I-1-b

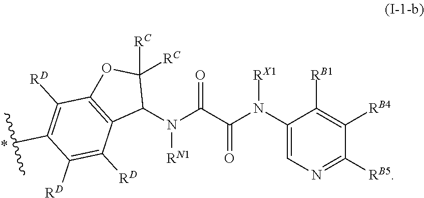

In some embodiments, T is:

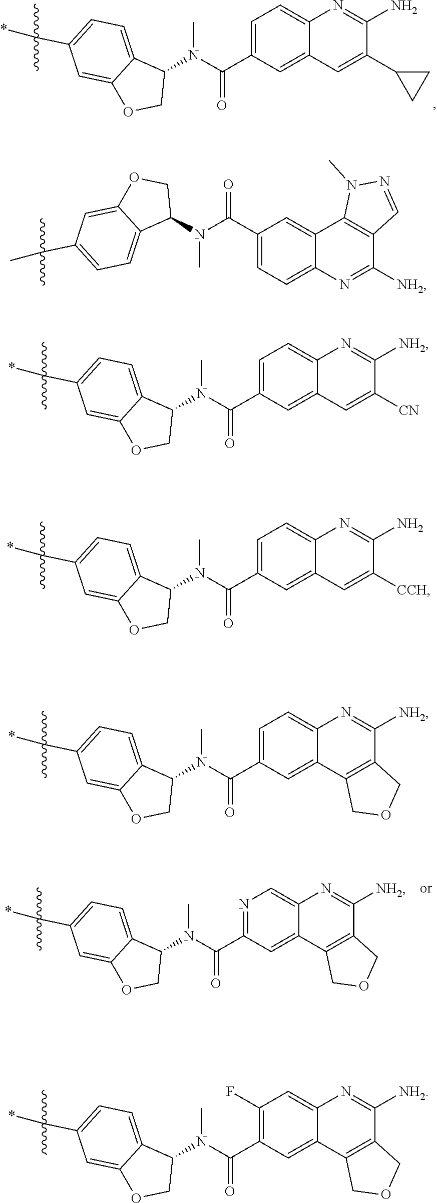

### Variable L

In some embodiments, at least one L′ is —C(═O)—.

In some embodiments, at least one L′ is —C(═O)N(R^{L′})—.

In some embodiments, at least one L′ is —N(R^{L′})C(═O)—.

In some embodiments, at least one L′ is —C(═O)O—.

In some embodiments, at least one L′ is —OC(═O)—.

In some embodiments, at least one L′ is —N(R^{L′})—.

In some embodiments, at least one L′ is —O—.

In some embodiments, at least one L′ is —O—(C₁₋₆ alkylene)—(e.g., —O-methylene-(C₁), —O-ethylene-(C₂), —O-n-propylene-(C₃), —O-i-propylene-(C₃), —O-n-butylene-(C₄), —O-i-butylene-(C₄), —O-s-butylene-(C₄), —O-t-butylene-(C₄), —O-pentylene-(C₅), or —O-hexylene-(C₆)) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, at least one L′ is —(C₁₋₆ alkylene)-O- (e.g., -methylene-O—(C₁), -ethylene-O—(C₂), -n-propylene-O—(C₃), -i-propylene-O—(C₃), -n-butylene-O—(C₄), -i-butylene-O—(C₄), -s-butylene-O—(C₄), -t-butylene-O—(C₄), -pentylene-O-(C₅), or -hexylene-O-(C₆)) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆).

In some embodiments, at least one L′ is —S—.

In some embodiments, at least one L′ is —S(═O)₂—.

In some embodiments, at least one L′ is C₁₋₆ alkylene (e.g., methylene (C₁), ethylene (C₂), n-propylene (C₃), i-propylene (C₃), n-butylene (C₄), i-butylene (C₄), s-butylene (C₄), t-butylene (C₄), pentylene (C₅), or hexylene (C₆)) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆).

In some embodiments, at least one L′ is C₁₋₆ heteroalkylene optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl (CA), i-butyl(C₄), s-butyl (CA), t-butyl(C₄), pentyl(C₅), or hexyl(C₆)).

In some embodiments, at least one L′ is C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, at least one L′ is C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆).

In some embodiments, at least one L′ is C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₅), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆).

In some embodiments, at least one L′ is 3- to 12-membered heterocyclylene (e.g., heterocyclylene comprising one or two 3- to 8-membered rings and 1-5 heteroatoms selected from N, O, and S) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, at least one L′ is C₆₋₁₀ arylene (e.g., phenylene or naphthylene) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, at least one L′ is 5- to 10-membered heteroarylene (e.g., heteroarylene comprising one or two 5- to 6-membered rings and 1-5 heteroatoms selected from N, O, and S) optionally substituted with one or more oxo, halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, wherein when at least one L′ is 5- to 10-membered heteroarylene (e.g., heteroarylene comprising one or two 5- to 6-membered rings and 1-5 heteroatoms selected from N, O, and S) optionally substituted with one or more oxo, then R^{B4} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, wherein when at least one L′ is 5- to 10-membered heteroarylene (e.g., heteroarylene comprising one or two 5- to 6-membered rings and 1-5 heteroatoms selected from N, O, and S) optionally substituted with one or more oxo, then R^{B5} is —NH₂.

In some embodiments, wherein when at least one L′ is 5- to 10-membered heteroarylene (e.g., heteroarylene comprising one or two 5- to 6-membered rings and 1-5 heteroatoms selected from N, O, and S) optionally substituted with one or more oxo, then R^{BN1} and R^{B4}, together with the intervening atoms to which they are attached, do not form 5-membered heteroaryl (e.g., heteroaryl comprising one 5-membered ring and 1-3 heteroatoms selected from N, O, and S) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₂), cyclooctyl(C₅), cyclooctenyl(C₅), bicyclo[2.2.1]heptanyl(C₇), bicyclo[2.2.2]octanyl(C₅), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), or 3- to 12-membered heterocyclyl (e.g., heterocyclyl comprising one or two 3- to 8-membered rings and 1-5 heteroatoms selected from N, O, and S).

In some embodiments, at least one R^{L′} is hydrogen.

In some embodiments, each R^{L′} is hydrogen.

In some embodiments, at least one R^{L′} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, at least one R^{L′} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, or —NH₂.

In some embodiments, at least one R^{E} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, or —NH₂.

In some embodiments, at least one R^{L′} is C₃₋₈ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₄), cyclooctenyl(C₅), bicyclo[2.2.1]heptanyl(C₂), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)) optionally substituted with one or more halogen (e.g., F, Cl, Br, or I), —CN, —NO₂, —OH, or —NH₂.

In some embodiments, at least one R^{L′} is 3- to 8-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, or —NH₂.

In some embodiments, at least one R^{L′} is —S(═O)₂R^{a} optionally substituted with one or more halogen (e.g., F, Cl, Br, or I), —CN, —NO₂, —OH, or —NH₂.

In some embodiments, at least one R^{L′} is —S(═O)₂OR^{a} optionally substituted with one or more halogen (e.g., F, Cl, Br, or I), —CN, —NO₂, —OH, or —NH₂.

In some embodiments, at least one R^{L′} is —S(═O)₂N(R^{a})₂ optionally substituted with one or more halogen (e.g., F, Cl, Br, or I), —CN, —NO₂, —OH, or —NH₂.

In some embodiments, at least one R^{L′} is —C(═O)R^{a} optionally substituted with one or more halogen (e.g., F, Cl, Br, or I), —CN, —NO₂, —OH, or —NH₂.

In some embodiments, at least one R^{L′} is —C(═O)OR^{a} optionally substituted with one or more halogen (e.g., F, Cl, Br, or I), —CN, —NO₂, —OH, or —NH₂.

In some embodiments, at least one RE′ is —C(═O)N(R^{a})₂ optionally substituted with one or more halogen (e.g., F, Cl, Br, or I), —CN, —NO₂, —OH, or —NH₂.

In some embodiments, at least one R^{a} is hydrogen.

In some embodiments, each R^{a} is hydrogen.

In some embodiments, at least one R^{a} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, at least one R^{a} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, or —NH₂.

In some embodiments, at least one R^{a} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, or —NH₂.

In some embodiments, 1 is an integer selected from 1 to 10, an integer selected from 2 to 10, an integer selected from 3 to 10, an integer selected from 4 to 10, an integer selected from 5 to 10, an integer selected from 6 to 10, an integer selected from 7 to 10, or an integer selected from 8 to 10.

In some embodiments, 1 is an integer selected from 1 to 9, an integer selected from 1 to 8, an integer selected from 1 to 7, an integer selected from 1 to 6, an integer selected from 1 to 5, an integer selected from 1 to 4, or an integer selected from 1 to 3.

In some embodiments, 1 is an integer selected from 2 to 9, an integer selected from 3 to 8, or an integer selected from 4 to 7.

In some embodiments, 1 is 0. In some embodiments, 1 is 1. In some embodiments, 1 is 2. In some embodiments, 1 is 3. In some embodiments, 1 is 4. In some embodiments, 1 is 5. In some embodiments, 1 is 6. In some embodiments, 1 is 7. In some embodiments, 1 is 8. In some embodiments, 1 is 9. In some embodiments, 1 is 10.

In some embodiments, each L′ is independently selected from —C(═O)—, —C(═O)N(R^{L′})—, —N(R^{L′})C(═O)—, —N(R^{L′})—, —O—, —O—(C₁₋₆ alkylene)-, —(C₁₋₆ alkylene)-O—, C₁₋₆ alkylene, C₁₋₆ heteroalkylene, C₃₋₁₂ carbocyclylene, 3- to 12-membered heterocyclylene, and 5- to 10-membered heteroarylene.

In some embodiments, L is of Formula I-2.

In some embodiments, L is of Formula I′-2.

In some embodiments, L is **—[O—(C₁₋₆ alkylene)]₁₋₈-N(R^{L′})C(—O)-(5- to 10-membered heteroarylene)-*, **—O—[(C₁₋₆ alkylene)]₁₋₃-N(R^{L′})C(═O)-(5- to 6-membered heteroarylene)-*, **—[O—(C₁₋₆ alkylene)]₁₋₇-(3- to 12-membered heterocyclylene)-C(═O)-(5- to 10-membered heteroarylene)-*, **—[O—(C₁₋₆ alkylene)]₁₋₆—O—(C₃₋₁₂ carbocyclylene)-N(R^{L′})C(═O)-(5- to 10-membered heteroarylene)-*, or **—O—[(C₁₋₆ heteroalkylene)]₁₋₃-N(R^{L′})C(═O)-(5- to 10-membered heteroarylene)-*

In some embodiments, L is **—[O—(C₁₋₆ alkylene)]₁₋₈-N(H)C(═O)-(pyridinylene)-*, **—O—[(C₁₋₆ alkylene)]₁₋₃-N(H)C(═O)-(pyridinylene)-*, **—[O—(C₁₋₆ alkylene)]₁₋₇-(3- to 12-membered heterocyclylene)-C(═O)-(pyridinylene)-*, **—[O—(C₁₋₆ alkylene)]₁₋₆—O—(C₃₋₁₂ carbocyclylene)-N (H)C(═O))-(pyridinylene)-*, or **—O—[(C₁₋₆ heteroalkylene)]₁₋₃-N(H)C(═O)-(pyridinylene)-*.

In some embodiments, L is **—O—(C₁₋₆ alkylene)-O—(C₃₋₁₂ carbocyclylene)-N(R^{L′})C(—O)-(5- to 10-membered heteroarylene)-*, **-(3- to 12-membered heterocyclylene)-(C₁₋₆ alkylene)-(3- to 12-membered heterocyclylene)-C(—O)-(5- to 10-membered heteroarylene)-*, or **-(3- to 12-membered heterocyclylene)-C(-0)-(3- to 12-membered heterocyclylene)-C(═O)-(5- to 10-membered heteroarylene)-*.

In some embodiments, L is **—O—(C₁₋₆ alkylene)-O—(C₃₋₁₂ carbocyclylene)-N(H)C(═O)-(pyridinylene)-*, **-(3- to 12-membered heterocyclylene)-(C₁₋₆ alkylene)-(3- to 12-membered heterocyclylene)-C(═O)-(pyridinylene)-*, or **-(3- to 12-membered heterocyclylene)-C(═O)-(3- to 12-membered heterocyclylene)-C(═O)-(pyridinylene)-*

In some embodiments, L is **-[L′]₁₋₁-(pyridinylene)-*

### Variable V

In some embodiments, R^{V1} is hydrogen.

In some embodiments, R^{V1} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I).

In some embodiments, R^{V1} is methyl.

In some embodiments, R^{V1} is C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₂), cycloheptadienyl(C₂), cycloheptatrienyl(C₂), cyclooctyl(C₈), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I).

In some embodiments, R^{V1} is C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I).

In some embodiments, R^{V1} is C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) optionally substituted with one or more halogen (e.g., F, Cl, Br, or I).

In some embodiments, R^{V1} is C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) substituted with one F.

In some embodiments, R^{V1} is 3- to 6-membered heterocyclyl (e.g., heterocyclyl comprising one 3- to 6-membered ring and 1-3 heteroatoms selected from N, O, and S) optionally substituted with one or more halogen (e.g., F, Cl, Br, or I), —CN, —NO₂, —OH, —NH₂, or C₁₋₆ alkyl (e.g., methyl (C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I).

In some embodiments, R^{V1} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) or C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)), wherein the C₁₋₆ alkyl or C₃₋₆ carbocyclyl is optionally substituted with one or more halogen (e.g., F, Cl, Br, or I).

In some embodiments, R^{VN1} is hydrogen.

In some embodiments, R^{VN1} is C₁₋₆ alkyl, wherein the C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) is optionally substituted with one or more halogen (e.g., F, Cl, Br, or I), —CN, —NO₂, —OH, or —NH₂.

In some embodiments, R^{V2} is hydrogen.

In some embodiments, R^{V2} is C₁₋₆ alkyl, wherein the C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) is optionally substituted with one or more halogen (e.g., F, Cl, Br, or I), —CN, —NO₂, —OH, —NH₂ or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{V2} is C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{V2} is 3- to 6-membered heterocyclyl (e.g., heterocyclyl comprising one 3- to 6-membered ring and 1-3 heteroatoms selected from N, O, and S) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{V2} is C₆₋₁₀ aryl (e.g., phenyl or naphthyl) optionally substituted with one or more halogen (e.g., F, Cl, Br, or I), —CN, —NO₂, —OH, —NH₂, or C₁₋₆ alkyl (e.g., methyl (C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, R^{V2} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, —NH₂, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)).

In some embodiments, at least one R^{A′} is hydrogen.

In some embodiments, each R^{A′} is hydrogen.

In some embodiments, at least one R^{A′} is halogen (e.g., F, Cl, Br, or I).

In some embodiments, at least one R^{A} is —CN.

In some embodiments, at least one R^{A′} is —NO₂.

In some embodiments, at least one R^{A′} is —OH.

In some embodiments, one R^{A′} is —OH, and each of the remaining R^{A′} is independently hydrogen.

In some embodiments, at least one R^{A′} is —NH₂.

In some embodiments, at least one R^{A′} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, at least one R^{A′} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{A′} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, butylhexylamino, i-butylhexylamino, s-butylhexylamino, t-butylhexylamino, of pentylhexylamino) optionally substituted with one or more halogen (e.g., F, Cl, Br, or I), —CN, —NO₂, —OH, or —NH₂.

In some embodiments, at least one R^{A′} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, or —NH₂.

In some embodiments, at least one R^{A′} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, or —NH₂.

In some embodiments, R^{VN2} is hydrogen.

In some embodiments, R^{VN2} is C₁₋₆ alkyl (e.g., methyl(C₁), ethyl(C₂), n-propyl(C₃), i-propyl(C₃), n-butyl (CA), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, at least one R^{V4} is hydrogen.

In some embodiments, each R^{V4} is hydrogen.

In some embodiments, at least one R^{V4} is halogen (e.g., F, Cl, Br, or I).

In some embodiments, at least one R^{V4} is —CN.

In some embodiments, at least one R^{V4} is —NO₂.

In some embodiments, at least one R^{V4} is —OH.

In some embodiments, at least one R^{V4} is —NH₂.

In some embodiments, at least one R^{V4} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, —NH₂, C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₂), cycloheptadienyl(C₇), cycloheptatrienyl(C₇), cyclooctyl(C₅), cyclooctenyl(C₄), bicyclo[2.2.1]heptanyl(C₇), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- to 6-membered rings and 1-5 heteroatoms selected from N, O, and S). In some embodiments, at least one R^{V4} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, —NH₂, C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₂), cycloheptatrienyl(C₇), cyclooctyl(C₅), cyclooctenyl(C₄), bicyclo[2.2.1]heptanyl(C₇), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- to 6-membered rings and 1-5 heteroatoms selected from N, O, and S).

In some embodiments, at least one R^{V4} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, of pentylhexylamino) optionally substituted with one or more halogen (e.g., F, Cl, Br, or I), —CN, —NO₂, —OH, —NH₂, C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₂), cycloheptatrienyl(C₂), cyclooctyl(C₄), cyclooctenyl(C₅), bicyclo[2.2.1]heptanyl (C), bicyclo[2.2.2]octanyl(C₄), cyclononyl(C₉), cyclononenyl(C₉), cyclodecyl(C₁₀), cyclodecenyl (C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- to 6-membered rings and 1-5 heteroatoms selected from N, O, and S).

In some embodiments, at least one R^{V4} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, —NH₂, C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₅), cyclooctenyl(C₅), bicyclo[2.2.1]heptanyl(C₇), bicyclo[2.2.2]octanyl(C₅), cyclononyl(C₉), cyclononenyl(C₅), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- to 6-membered rings and 1-5 heteroatoms selected from N, O, and S).

In some embodiments, at least one R^{V4} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, —NH₂, C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₂), cycloheptatrienyl(C₇), cyclooctyl(C₈), cyclooctenyl(C₄), bicyclo[2.2.1]heptanyl(C₂), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- to 6-membered rings and 1-5 heteroatoms selected from N, O, and S).

In some embodiments, at least one R^{V5} is hydrogen.

In some embodiments, each R^{V5} is hydrogen.

In some embodiments, at least one R^{V5} is halogen (e.g., F, Cl, Br, or I).

In some embodiments, at least one R^{V5} is —CN.

In some embodiments, at least one R^{V5} is —NO₂.

In some embodiments, at least one R^{V5} is —OH.

In some embodiments, at least one R^{V5} is —NH₂.

In some embodiments, at least one R^{V5} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, or —NH₂.

In some embodiments, at least one R^{V5} is C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)) optionally substituted with one or more halogen (e.g., F, Cl, Br, or I), —CN, —NO₂, —OH, or —NH₂.

In some embodiments, at least one R^{V5} is C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-t-propylamino, methyl-n-butylamino, methyl-t-butylamino, methyl-s-butylamino, methyl-t-butylamino, methylpentylamino, methylhexylamino, ethyl-n-propylamino, ethyl-t-propylamino, ethyl-n-butylamino, ethyl-s-butylamino, ethyl-t-butylamino, ethyl-t-butylamino, ethylpentylamino, ethylhexylamino, propyl-n-butylamino, propyl-i-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino) optionally substituted with one or more halogen (e.g., F, Cl, Br, or I), —CN, —NO₂, —OH, or —NH₂.

In some embodiments, at least one R^{V5} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, or —NH₂.

In some embodiments, at least one R^{V5} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, or —NH₂.

In some embodiments, R^{V6} is hydrogen.

In some embodiments, R^{V6} is halogen (e.g., F, Cl, Br, or I).

In some embodiments, R^{V6} is —CN.

In some embodiments, R^{V6} is C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more —CN, C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), or C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, R^{V6} is C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), S-butyl(C₄), t-butyl(C₄), pentyl(C₅), or hexyl(C₆)) optionally substituted with one or more-CN, C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), or C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, R^{V6} is C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more-CN, C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), or C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, R^{V6} is C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₂), cycloheptenyl(C₇), cycloheptadienyl(C₇), cycloheptatrienyl(C₂), cyclooctyl(C₄), cyclooctenyl(C₈), bicyclo[2.2.1]heptanyl(C₇), bicyclo[2.2.2]octanyl(C₄), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl (CA), i-butyl(C₄), s-butyl(C₄), t-butyl(C₄), pentyl(C₅), or hexyl(C₆)) optionally substituted with one or more-CN, C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), or C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, R^{V6} is 3- to 12-membered heterocyclyl (e.g., heterocyclyl comprising one or two 3- to 8-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more-CN, C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), or C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, R^{V6} is C₆₋₁₀ aryl (e.g., phenyl or naphthyl) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more-CN, C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), or C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, R^{V6} is 5- to 10-membered heteroaryl (e.g., heteroaryl comprising one or two 5- to 6-membered rings and 1-5 heteroatoms selected from N, O, and S) optionally substituted with one or more halogen (e.g., F, Cl, Br, or I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more-CN, C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆), or C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)).

In some embodiments, R^{V6} is C₂₋₆ alkynyl or 5- to 10-membered heteroaryl (e.g., heteroaryl comprising one or two 5- to 6-membered rings and 1-5 heteroatoms selected from N, O, and S) optionally substituted with one or more C₁₋₆ alkyl e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆).

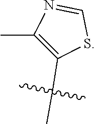 [SMILES: *c1scnc1C]

In some embodiments, R^{V6} is —C≡CH or

In some embodiments, V is of Formula I-3.

In some embodiments, V is of Formula I′-3.

In some embodiments, V is of Formula I-3-a

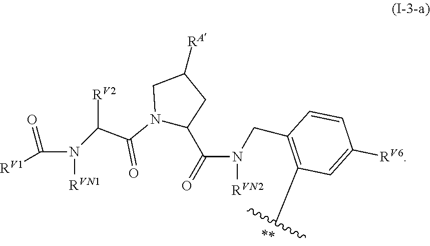

In some embodiments, V is of Formula I-3-a-i

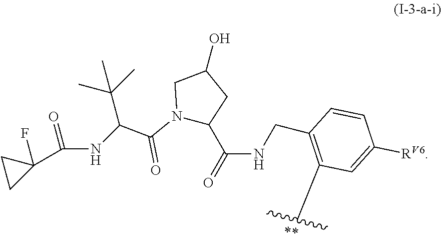

In some embodiments, V is

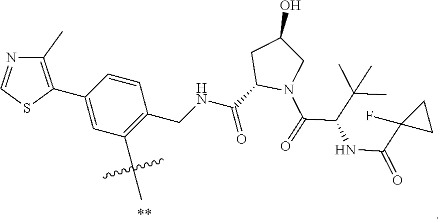 [SMILES: *c1cc(-c2scnc2C)ccc1CNC(=O)[C@@H]1C[C@@H](O)CN1C(=O)[C@@H](NC(=O)C1(F)CC1)C(C)(C)C]

Embodiments of the variables in any of the Formulae described herein, e.g., Formulae I-1, I′-1, 1-2, I′-2, 1-3, and I′-3, as applicable, are described below. Any of the variables can be any moiety as described in the embodiments below. In addition, the combination of any moieties described for any of the variables, as applicable, with any moieties described for any of the remaining variables, are also contemplated.

Without wishing to be limited by this statement, while various options for variables are described herein, it is understood that the present disclosure intends to encompass operable embodiments having combinations of the options. The disclosure may be interpreted as excluding the non-operable embodiments caused by certain combinations of the options.

When a range of values is listed, each discrete value and sub-range within the range are also contemplated. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

In some embodiments, the compound is selected from the compounds in Table 1.

In some embodiments, the compound is selected from the compounds in Table 1, or a pharmaceutically acceptable salt thereof.

In some embodiments, the compound is selected from the compounds in Table 1, or a pharmaceutically acceptable salt, solvate, or stereoisomer thereof.

TABLE 1

| Com- | |
|---|---|
| pound | |
| No. | Structure |
| A1 | 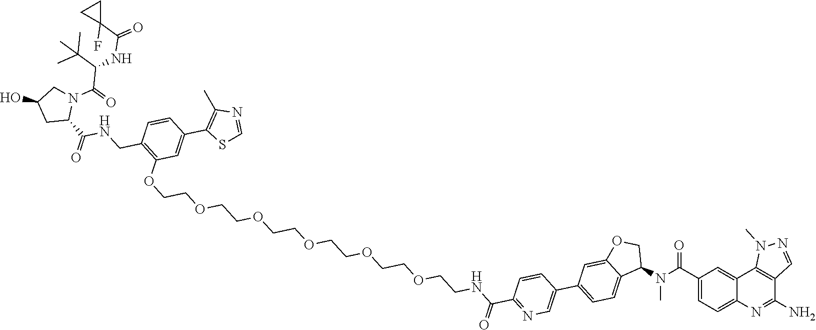 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)c2ccc(-c3ccc4c(c3)OC[C@H]4N(C)C(=O)c3ccc4nc(N)c5cnn(C)c5c4c3)cn2)c1] |
| A2 | 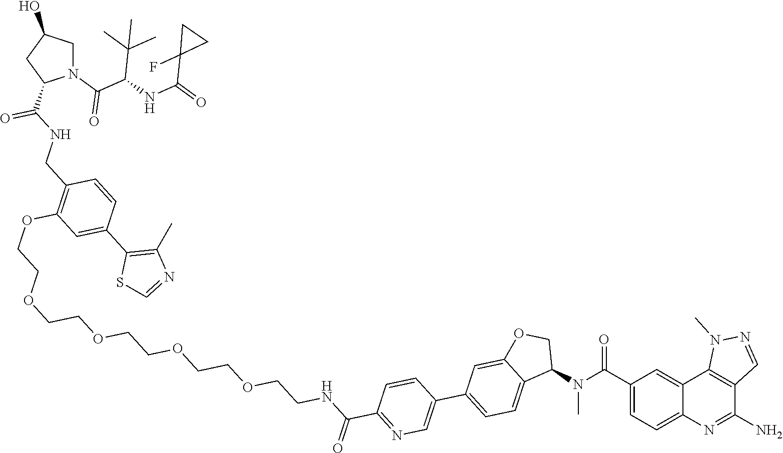 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)c2ccc(-c3ccc4c(c3)OC[C@H]4N(C)C(=O)c3ccc4nc(N)c5cnn(C)c5c4c3)cn2)c1] |
| A3 | 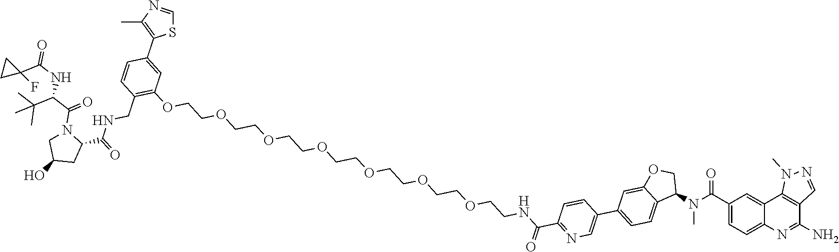 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCOCCNC(=O)c2ccc(-c3ccc4c(c3)OC[C@H]4N(C)C(=O)c3ccc4nc(N)c5cnn(C)c5c4c3)cn2)c1] |
| A4 | 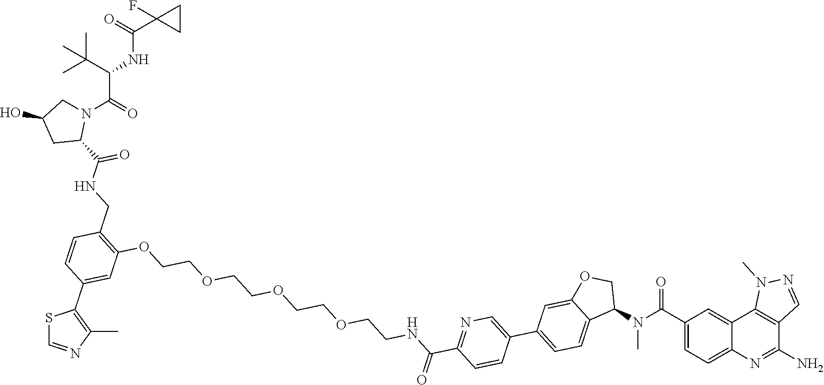 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3ccc4nc(N)c5cnn(C)c5c4c3)cn2)c1] |
| A5 | 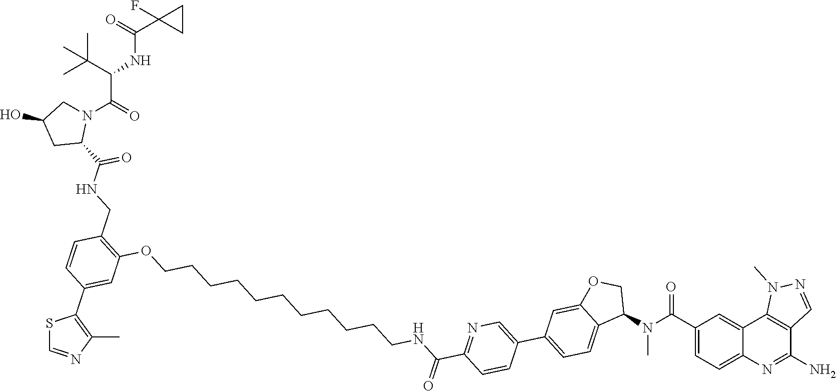 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCNC(=O)c2ccc(-c3ccc4c(c3)OC[C@H]4N(C)C(=O)c3ccc4nc(N)c5cnn(C)c5c4c3)cn2)c1] |
| A6 | 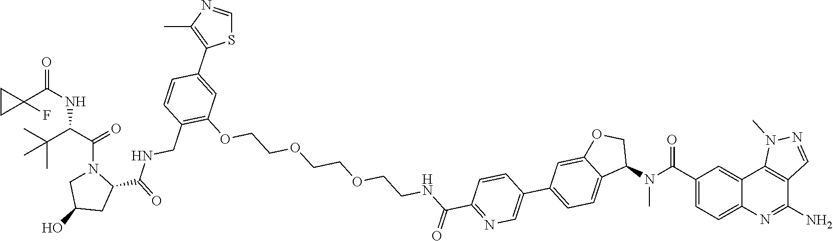 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c5cnn(C)c5c4c3)cn2)c1] |
| A7 | 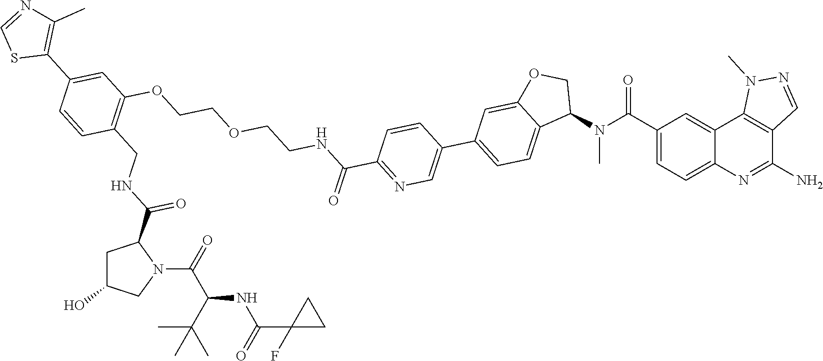 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCNC(=O)c2ccc(-c3ccc4c(c3)OC[C@H]4N(C)C(=O)c3ccc4nc(N)c5cnn(C)c5c4c3)cn2)c1] |
| A8 | 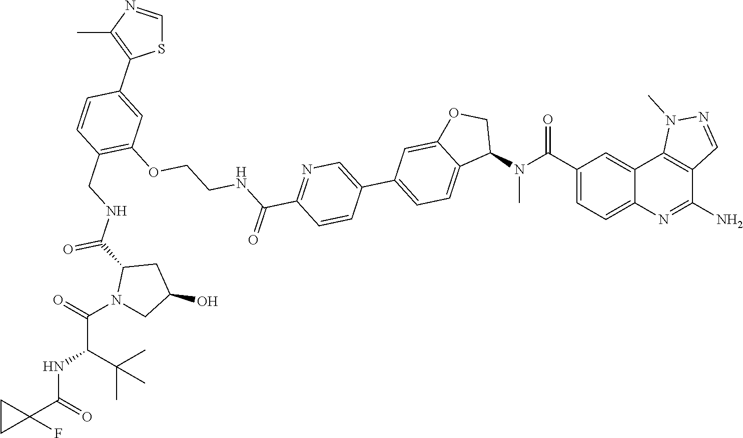 [SMILES: Cc1ncsc1-c1ccc(CNC(=O)[C@@H]2C[C@@H](O)CN2C(=O)[C@@H](NC(=O)C2(F)CC2)C(C)(C)C)c(OCCNC(=O)c2ccc(-c3ccc4c(c3)OC[C@H]4N(C)C(=O)c3ccc4nc(N)c5cnn(C)c5c4c3)cn2)c1] |
| A9 | 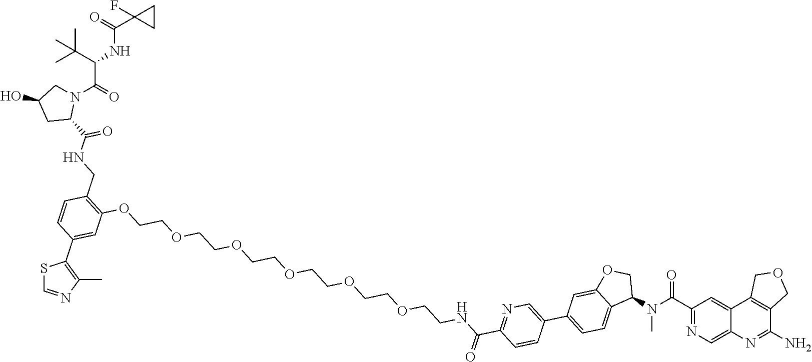 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)c2ccc(-c3ccc4c(c3)OC[C@H]4N(C)C(=O)c3cc4c5c(c(N)nc4cn3)COC5)cn2)c1] |
| A10 | 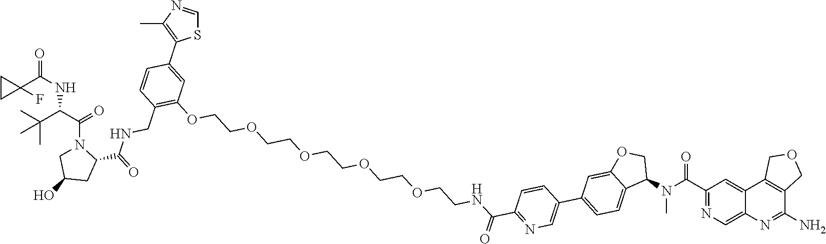 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)c2ccc(-c3ccc4c(c3)OC[C@H]4N(C)C(=O)c3cc4c5c(c(N)nc4cn3)COC5)cn2)c1] |
| A11 | 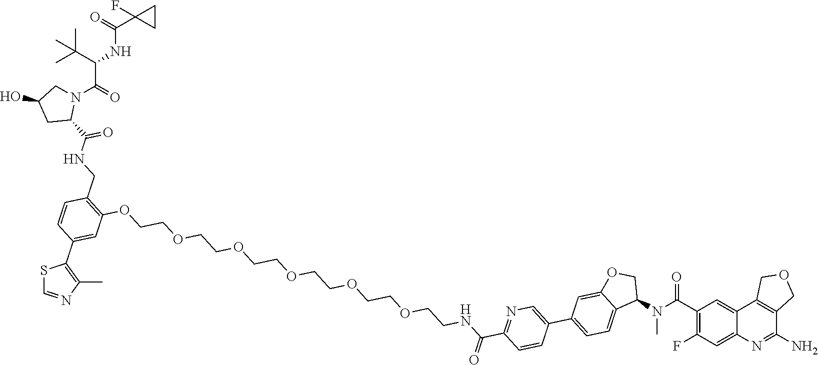 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)c2ccc(-c3ccc4c(c3)OC[C@H]4N(C)C(=O)c3cc4c5c(c(N)nc4cc3F)COC5)cn2)c1] |
| A12 | 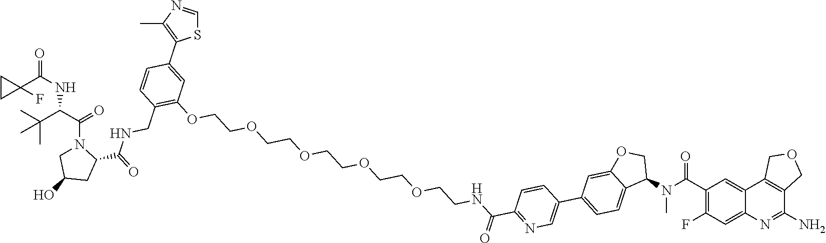 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)c2ccc(-c3ccc4c(c3)OC[C@H]4N(C)C(=O)c3cc4c5c(c(N)nc4cc3F)COC5)cn2)c1] |
| A13 | 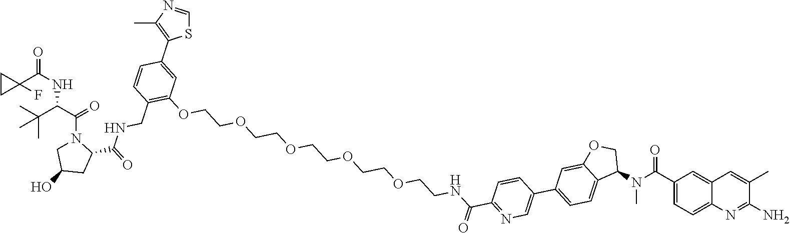 [SMILES: Cc1cc2cc(C(=O)N(C)[C@@H]3COc4cc(-c5ccc(C(=O)NCCOCCOCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A14 | 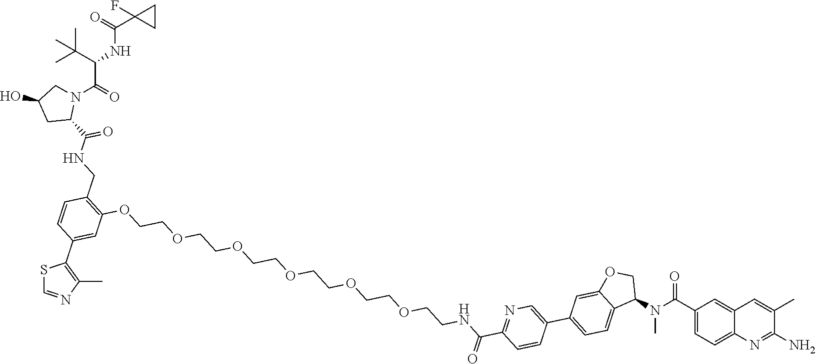 [SMILES: Cc1cc2cc(C(=O)N(C)[C@@H]3COc4cc(-c5ccc(C(=O)NCCOCCOCCOCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A15 | 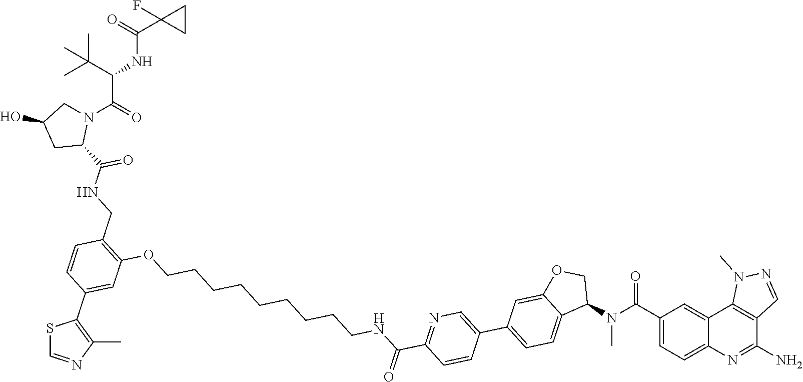 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCNC(=O)c2ccc(-c3ccc4c(c3)OC[C@H]4N(C)C(=O)c3ccc4nc(N)c5cnn(C)c5c4c3)cn2)c1] |
| A16 | 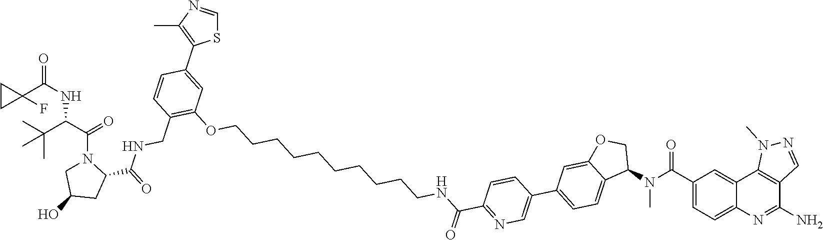 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCNC(=O)c2ccc(-c3ccc4c(c3)OC[C@H]4N(C)C(=O)c3ccc4nc(N)c5cnn(C)c5c4c3)cn2)c1] |
| A17 | 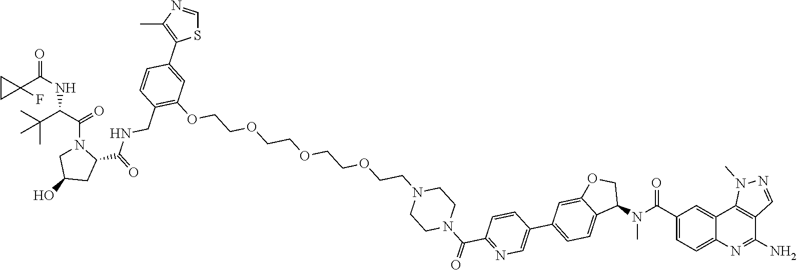 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCN2CCN(C(=O)c3ccc(-c4ccc5c(c4)OC[C@H]5N(C)C(=O)c4ccc5nc(N)c6cnn(C)c6c5c4)cn3)CC2)c1] |
| A18 | 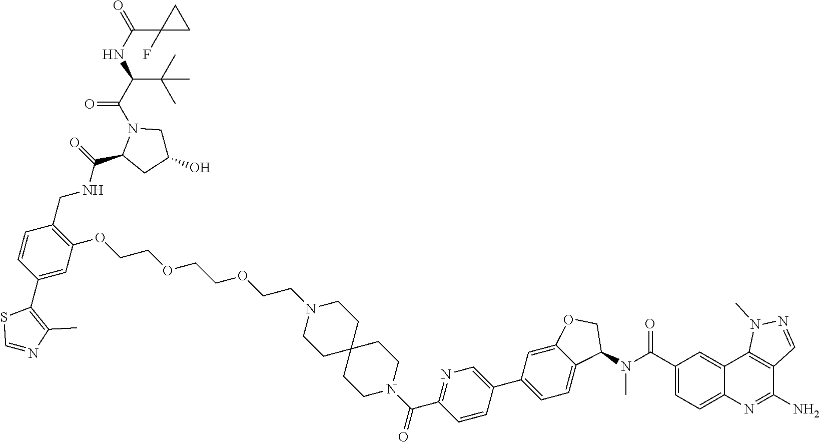 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCN2CCC3(CC2)CCN(C(=O)c2ccc(-c4ccc5c(c4)OC[C@H]5N(C)C(=O)c4ccc5nc(N)c6cnn(C)c6c5c4)cn2)CC3)c1] |
| A19 | 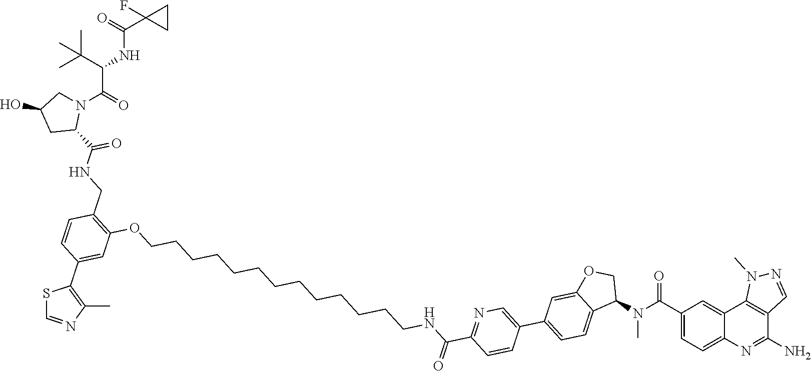 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCNC(=O)c2ccc(-c3ccc4c(c3)OC[C@H]4N(C)C(=O)c3ccc4nc(N)c5cnn(C)c5c4c3)cn2)c1] |
| A20 | 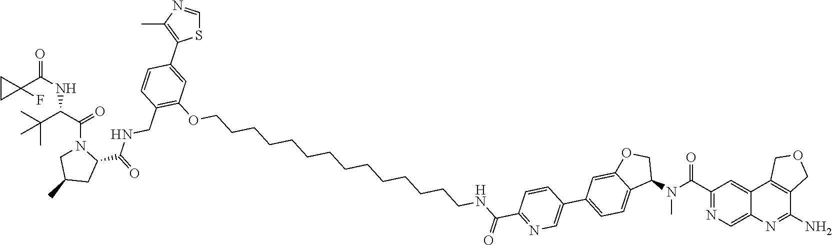 [SMILES: Cc1ncsc1-c1ccc(CNC(=O)[C@@H]2C[C@@H](C)CN2C(=O)[C@@H](NC(=O)C2(F)CC2)C(C)(C)C)c(OCCCCCCCCCCCCCCNC(=O)c2ccc(-c3ccc4c(c3)OC[C@H]4N(C)C(=O)c3cc4c5c(c(N)nc4cn3)COC5)cn2)c1] |
| A21 | 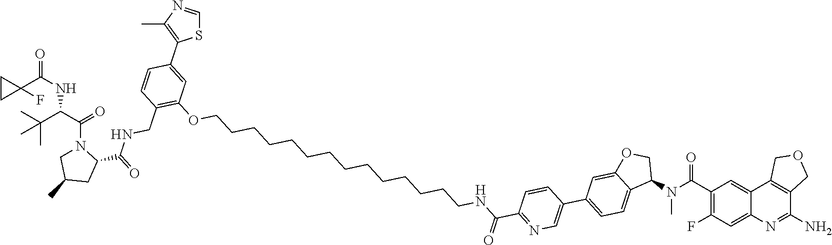 [SMILES: Cc1ncsc1-c1ccc(CNC(=O)[C@@H]2C[C@@H](C)CN2C(=O)[C@@H](NC(=O)C2(F)CC2)C(C)(C)C)c(OCCCCCCCCCCCCCCNC(=O)c2ccc(-c3ccc4c(c3)OC[C@H]4N(C)C(=O)c3cc4c5c(c(N)nc4cc3F)COC5)cn2)c1] |
| A22 | 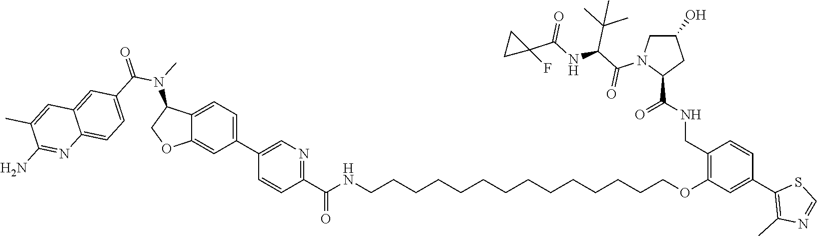 [SMILES: Cc1cc2cc(C(=O)N(C)[C@@H]3COc4cc(-c5ccc(C(=O)NCCCCCCCCCCCCCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A23 | 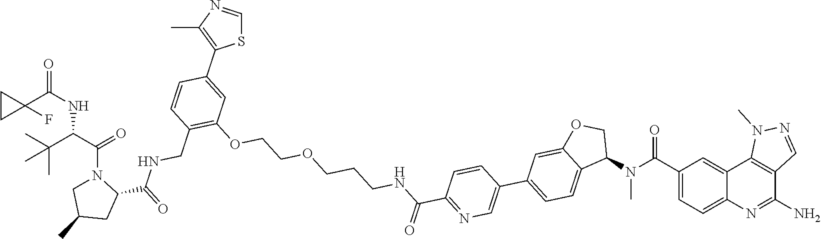 [SMILES: Cc1ncsc1-c1ccc(CNC(=O)[C@@H]2C[C@@H](C)CN2C(=O)[C@@H](NC(=O)C2(F)CC2)C(C)(C)C)c(OCCOCCCNC(=O)c2ccc(-c3ccc4c(c3)OC[C@H]4N(C)C(=O)c3ccc4nc(N)c5cnn(C)c5c4c3)cn2)c1] |
| A24 | 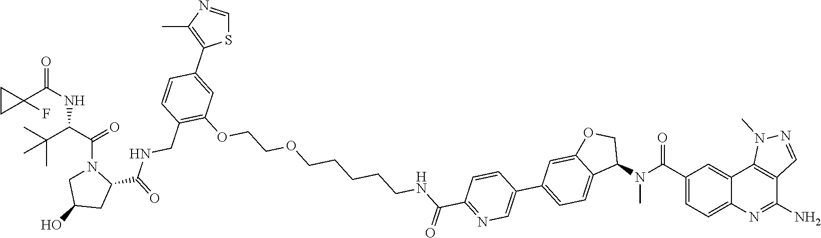 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCCNC(=O)c2ccc(-c3ccc4c(c3)OC[C@H]4N(C)C(=O)c3ccc4nc(N)c5cnn(C)c5c4c3)cn2)c1] |
| A25 | 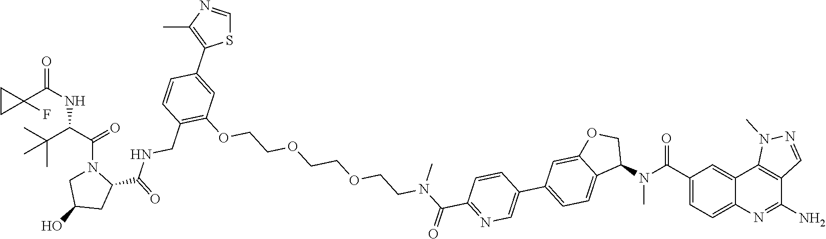 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCN(C)C(=O)c2ccc(-c3ccc4c(c3)OC[C@H]4N(C)C(=O)c3ccc4nc(N)c5cnn(C)c5c4c3)cn2)c1] |
| A26 | 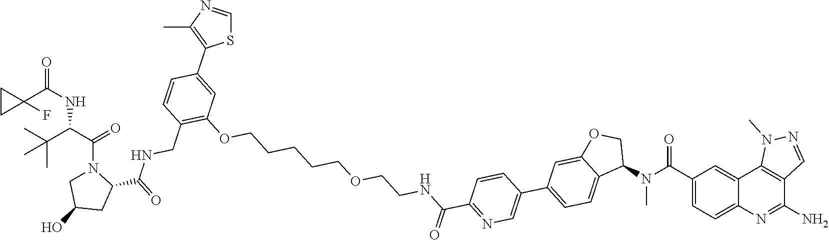 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCOCCNC(=O)c2ccc(-c3ccc4c(c3)OC[C@H]4N(C)C(=O)c3ccc4nc(N)c5cnn(C)c5c4c3)cn2)c1] |
| A27 | 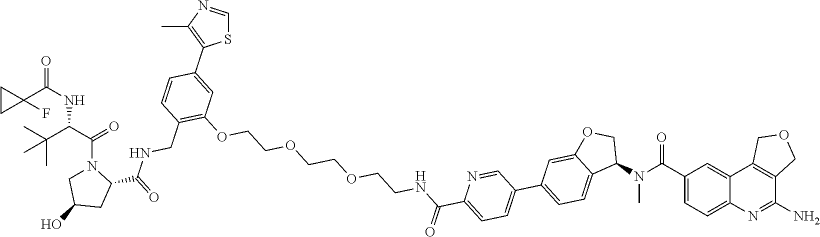 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c5c(c4c3)COC5)cn2)c1] |
| A28 | 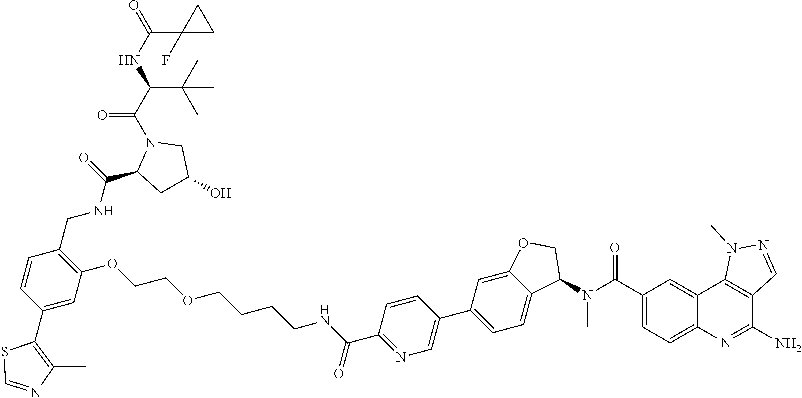 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c5cnn(C)c5c4c3)cn2)c1] |
| A29 | 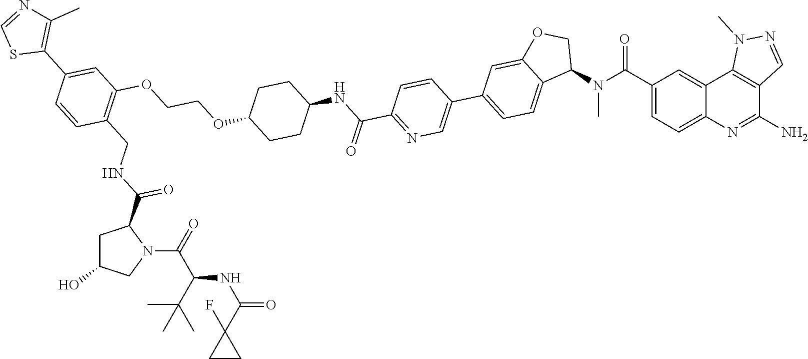 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6cnn(C)c6c5c4)cn3)CC2)c1] |
| A30 | 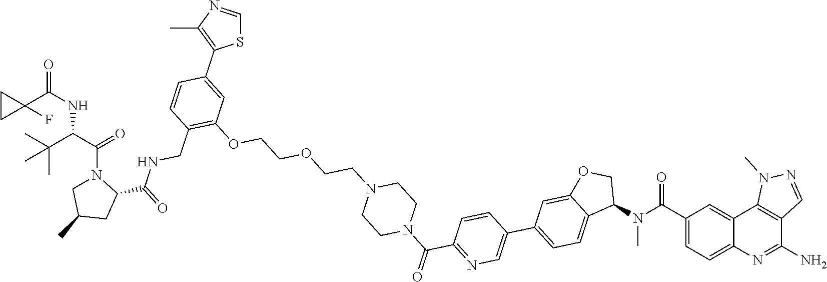 [SMILES: Cc1ncsc1-c1ccc(CNC(=O)[C@@H]2C[C@@H](C)CN2C(=O)[C@@H](NC(=O)C2(F)CC2)C(C)(C)C)c(OCCOCCN2CCN(C(=O)c3ccc(-c4ccc5c(c4)OC[C@H]5N(C)C(=O)c4ccc5nc(N)c6cnn(C)c6c5c4)cn3)CC2)c1] |
| A31 | 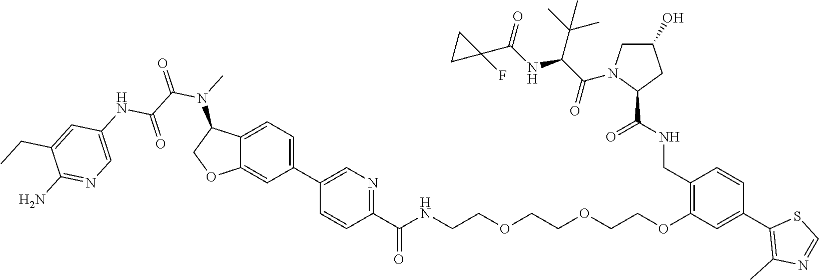 [SMILES: CCc1cc(NC(=O)C(=O)N(C)[C@@H]2COc3cc(-c4ccc(C(=O)NCCOCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A32 | 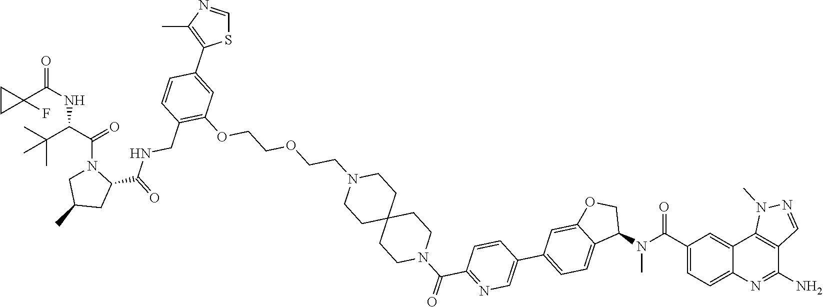 [SMILES: Cc1ncsc1-c1ccc(CNC(=O)[C@@H]2C[C@@H](C)CN2C(=O)[C@@H](NC(=O)C2(F)CC2)C(C)(C)C)c(OCCOCCN2CCC3(CC2)CCN(C(=O)c2ccc(-c4ccc5c(c4)OC[C@H]5N(C)C(=O)c4ccc5nc(N)c6cnn(C)c6c5c4)cn2)CC3)c1] |
| A33 | 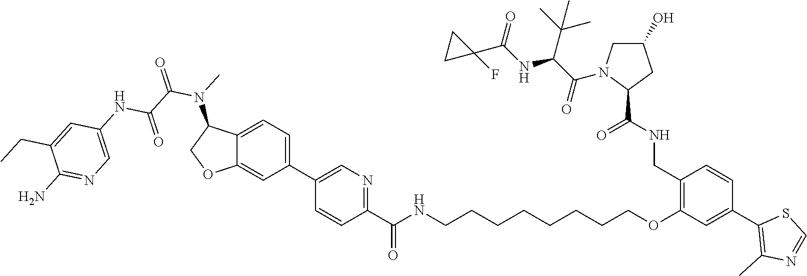 [SMILES: CCc1cc(NC(=O)C(=O)N(C)[C@@H]2COc3cc(-c4ccc(C(=O)NCCCCCCCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A34 | 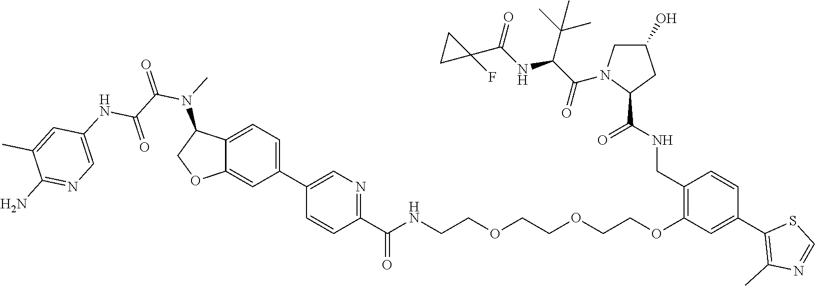 [SMILES: Cc1cc(NC(=O)C(=O)N(C)[C@@H]2COc3cc(-c4ccc(C(=O)NCCOCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A35 | 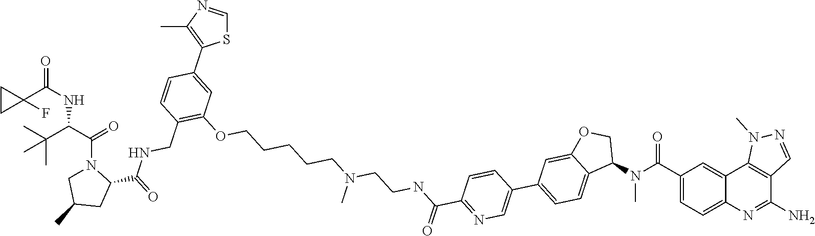 [SMILES: Cc1ncsc1-c1ccc(CNC(=O)[C@@H]2C[C@@H](C)CN2C(=O)[C@@H](NC(=O)C2(F)CC2)C(C)(C)C)c(OCCCCCN(C)CCNC(=O)c2ccc(-c3ccc4c(c3)OC[C@H]4N(C)C(=O)c3ccc4nc(N)c5cnn(C)c5c4c3)cn2)c1] |
| A36 | 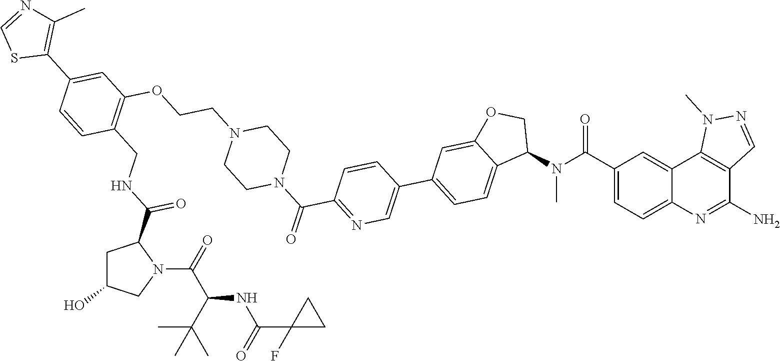 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(C(=O)c3ccc(-c4ccc5c(c4)OC[C@H]5N(C)C(=O)c4ccc5nc(N)c6cnn(C)c6c5c4)cn3)CC2)c1] |
| A37 | 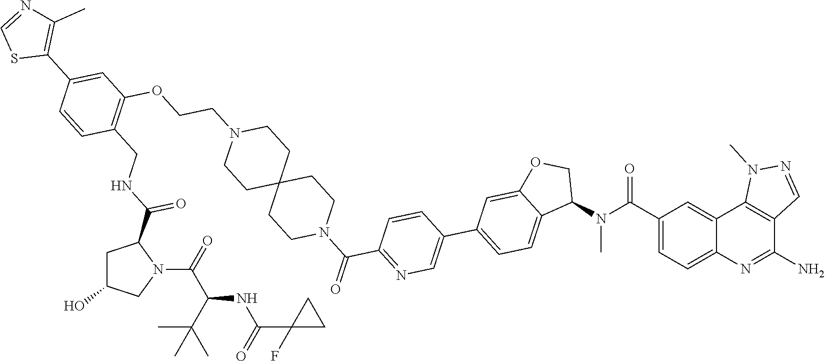 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC3(CC2)CCN(C(=O)c2ccc(-c4ccc5c(c4)OC[C@H]5N(C)C(=O)c4ccc5nc(N)c6cnn(C)c6c5c4)cn2)CC3)c1] |
| A38 | 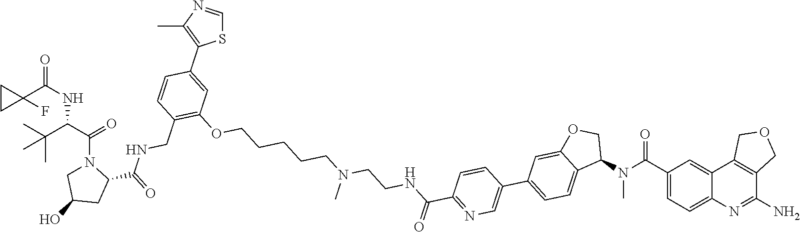 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCN(C)CCNC(=O)c2ccc(-c3ccc4c(c3)OC[C@H]4N(C)C(=O)c3ccc4nc(N)c5c(c4c3)COC5)cn2)c1] |
| A39 | 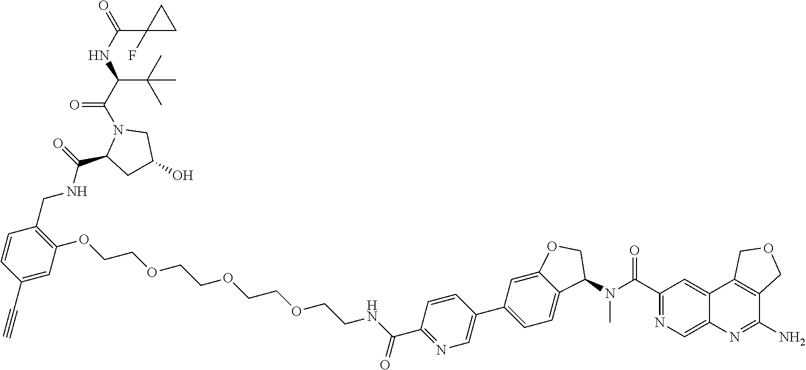 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3cc4c5c(c(N)nc4cn3)COC5)cn2)c1] |
| A40 | 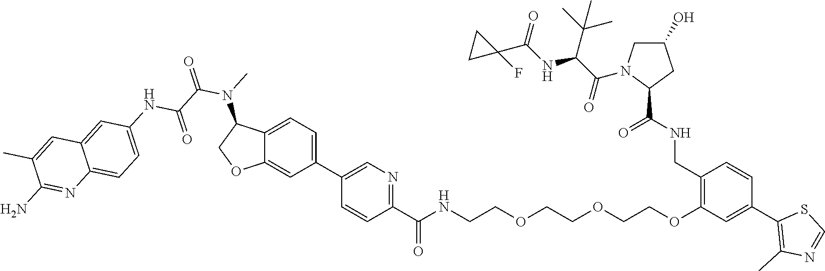 [SMILES: Cc1cc2cc(NC(=O)C(=O)N(C)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A41 | 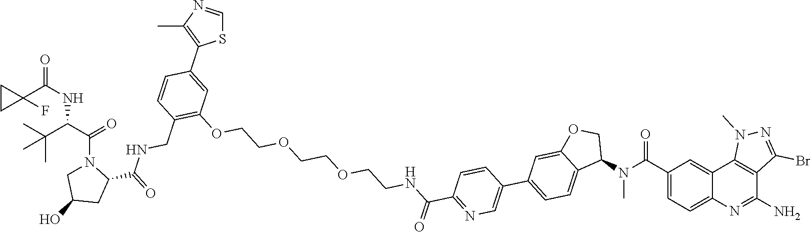 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c5c(Br)nn(C)c5c4c3)cn2)c1] |
| A42 | 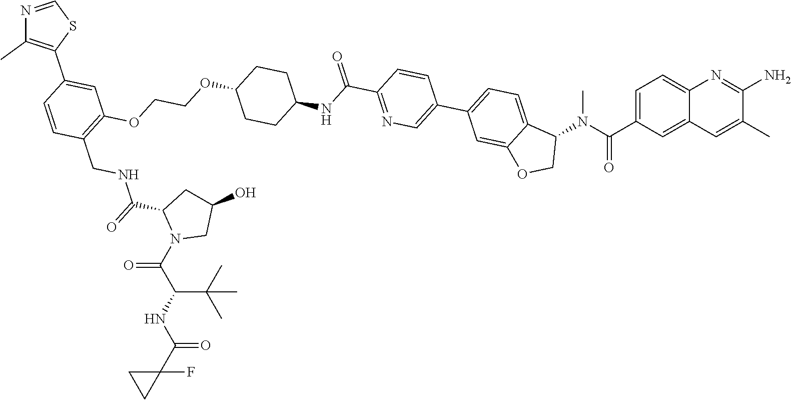 [SMILES: Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A43 | 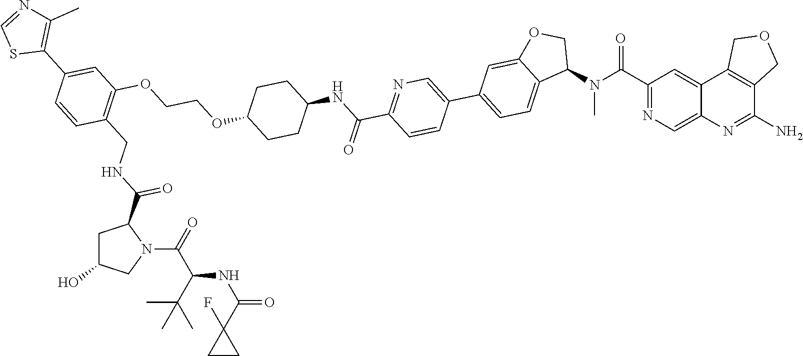 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4cc5c6c(c(N)nc5cn4)COC6)cn3)CC2)c1] |
| A44 | 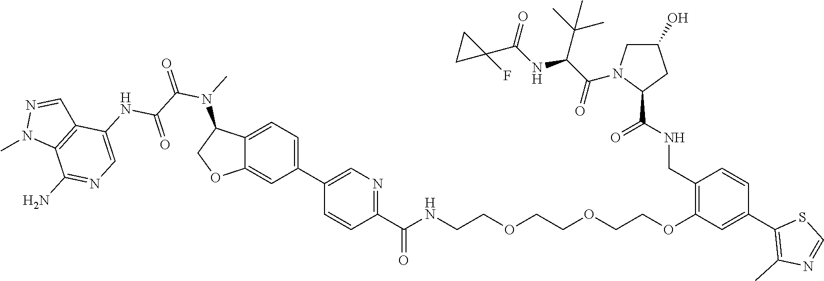 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)C(=O)Nc3cnc(N)c4c3cnn4C)cn2)c1] |
| A45 | 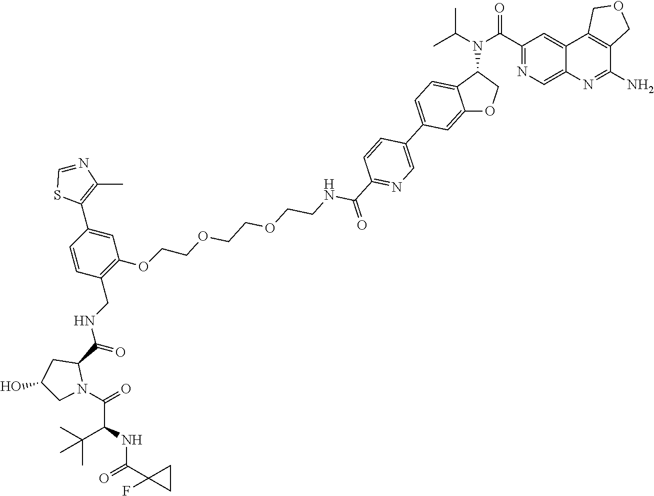 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C(=O)c3cc4c5c(c(N)nc4cn3)COC5)C(C)C)cn2)c1] |
| A46 | 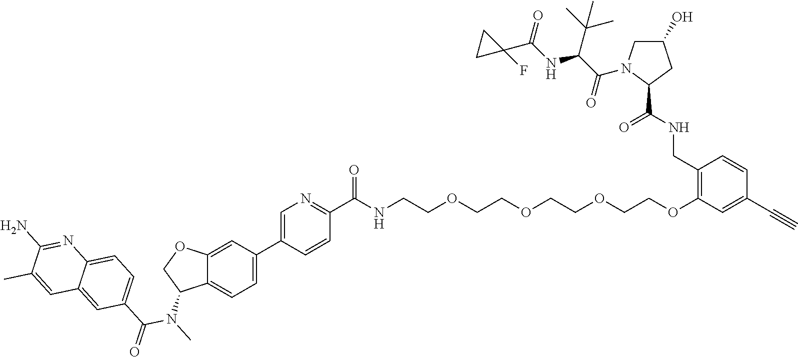 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3ccc4nc(N)c(C)cc4c3)cn2)c1] |
| A47 | 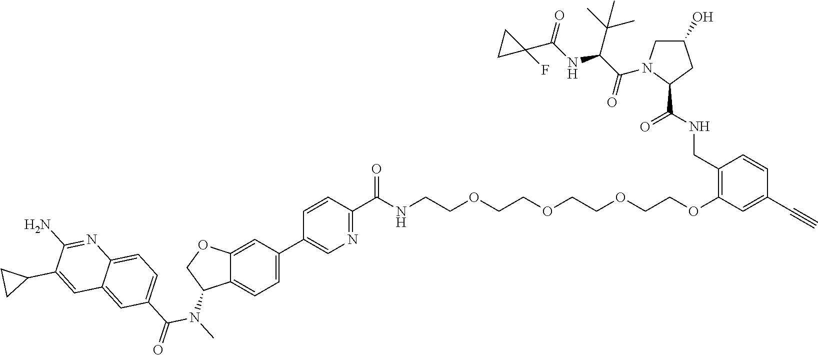 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3ccc4nc(N)c(C5CC5)cc4c3)cn2)c1] |
| A48 | 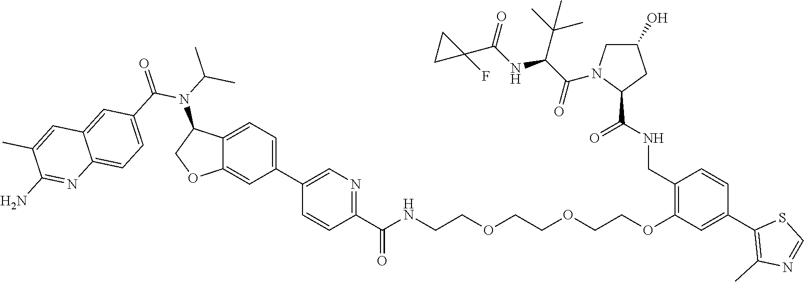 [SMILES: Cc1cc2cc(C(=O)N(C(C)C)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A49 | 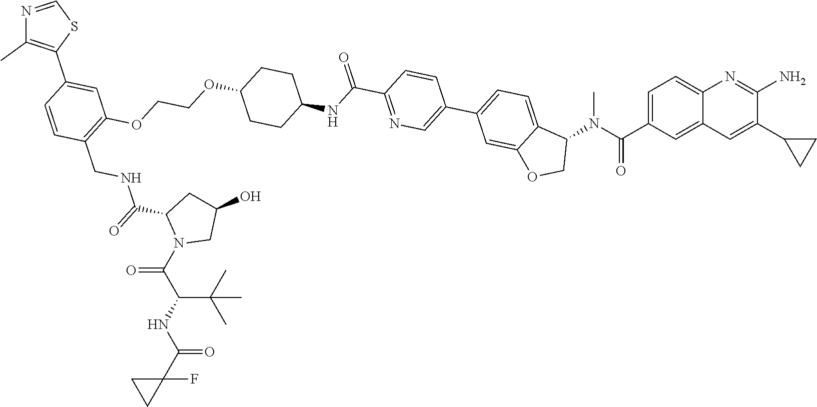 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CC6)cc5c4)cn3)CC2)c1] |
| A50 | 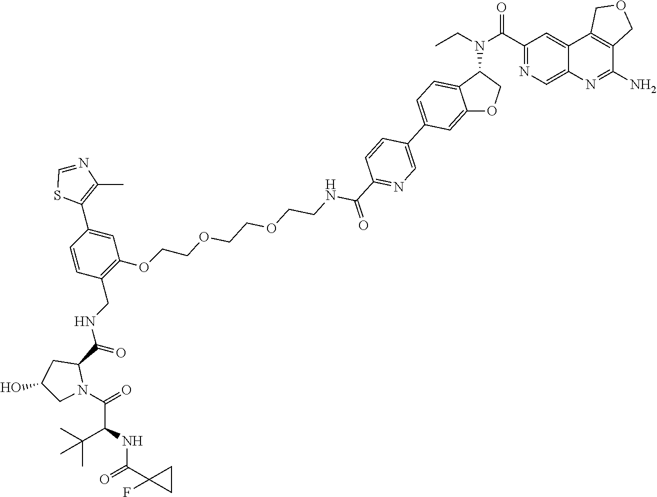 [SMILES: CCN(C(=O)c1cc2c3c(c(N)nc2cn1)COC3)[C@@H]1COc2cc(-c3ccc(C(=O)NCCOCCOCCOc4cc(-c5scnc5C)ccc4CNC(=O)[C@@H]4C[C@@H](O)CN4C(=O)[C@@H](NC(=O)C4(F)CC4)C(C)(C)C)nc3)ccc21] |
| A51 | 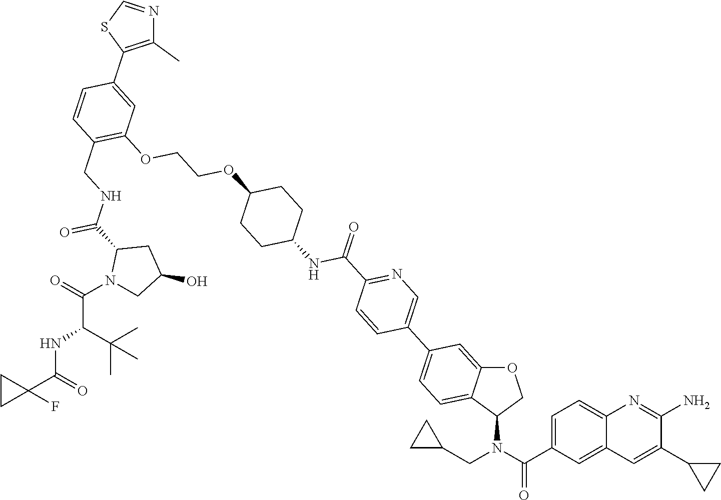 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(CC4CC4)C(=O)c4ccc5nc(N)c(C6CC6)cc5c4)cn3)CC2)c1] |
| A52 | 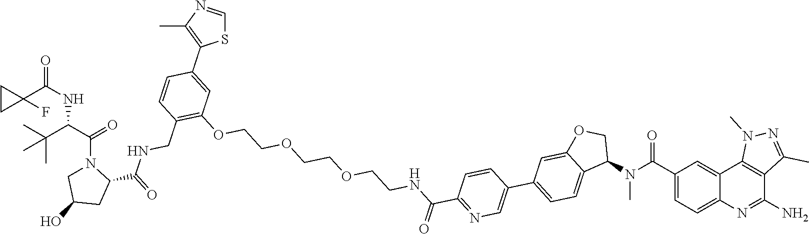 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c5c(C)nn(C)c5c4c3)cn2)c1] |
| A53 | 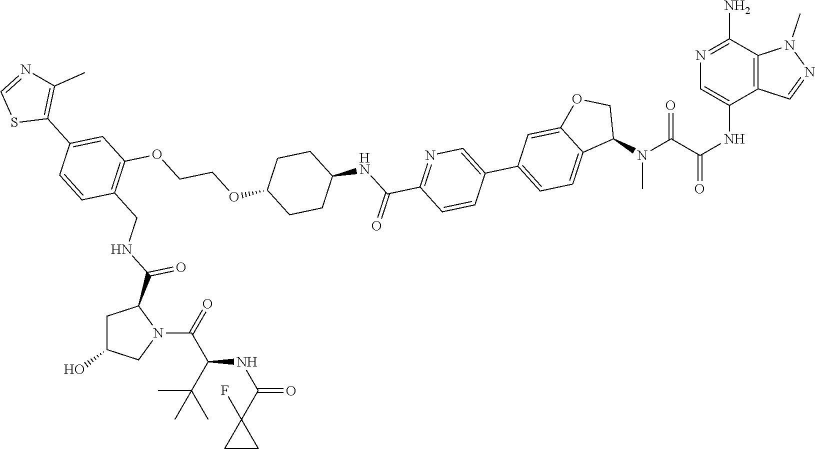 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)C(=O)Nc4cnc(N)c5c4cnn5C)cn3)CC2)c1] |
| A54 | 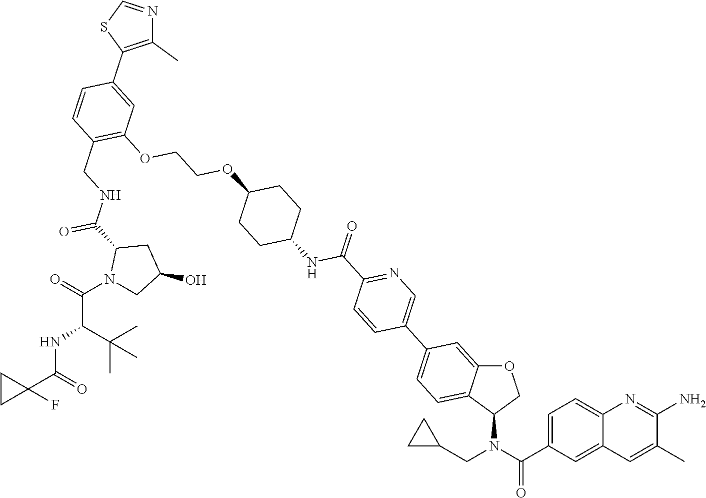 [SMILES: Cc1cc2cc(C(=O)N(CC3CC3)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A55 | 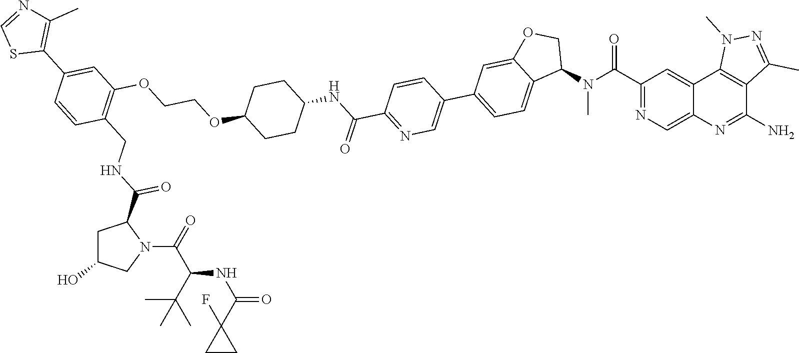 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4cc5c(cn4)nc(N)c4c(C)nn(C)c45)cn3)CC2)c1] |
| A56 | 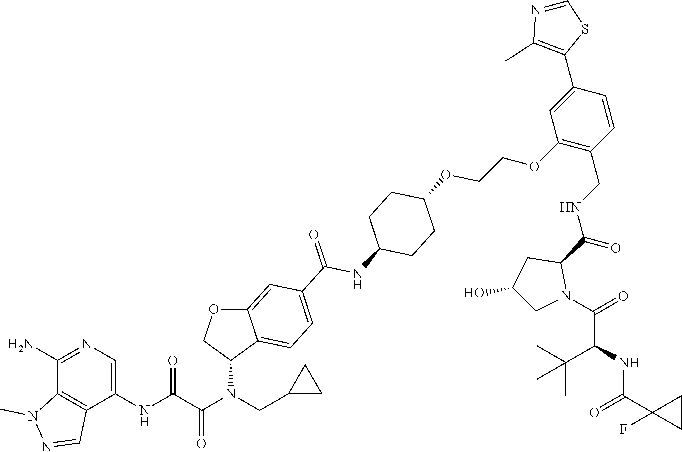 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc4c(c3)OC[C@H]4N(CC3CC3)C(=O)C(=O)Nc3cnc(N)c4c3cnn4C)CC2)c1] |
| A57 | 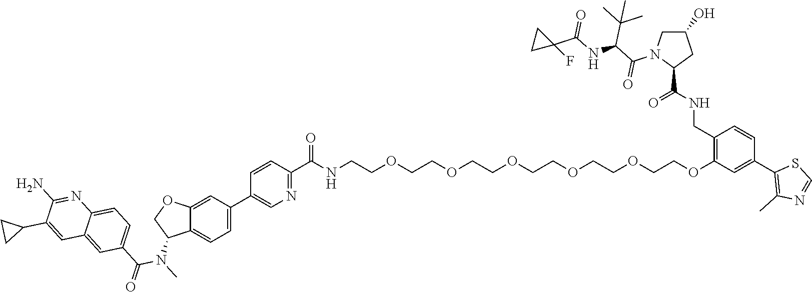 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)c2ccc(-c3ccc4c(c3)OC[C@H]4N(C)C(=O)c3ccc4nc(N)c(C5CC5)cc4c3)cn2)c1] |
| A58 | 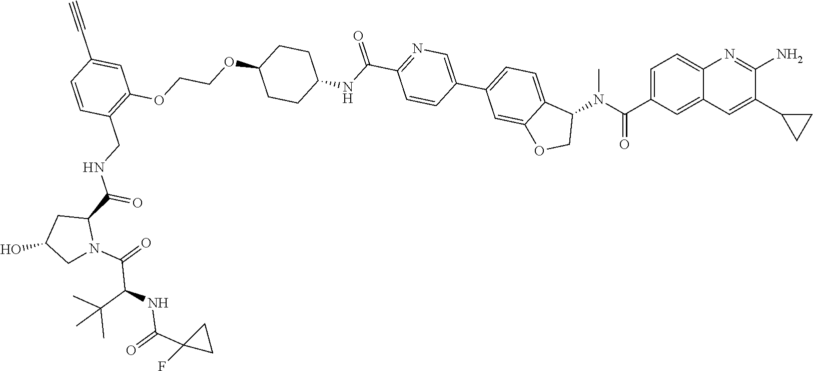 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CC6)cc5c4)cn3)CC2)c1] |
| A59 | 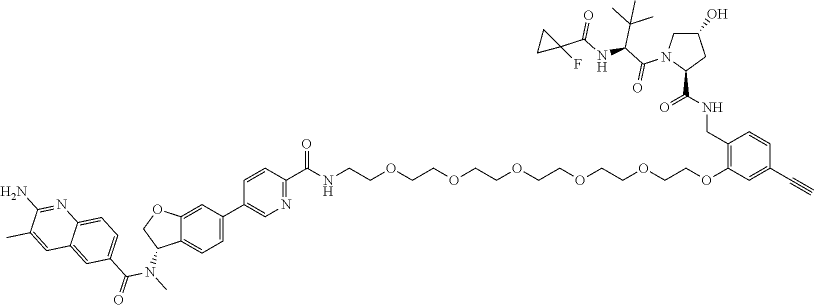 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)c2ccc(-c3ccc4c(c3)OC[C@H]4N(C)C(=O)c3ccc4nc(N)c(C)cc4c3)cn2)c1] |
| A60 | 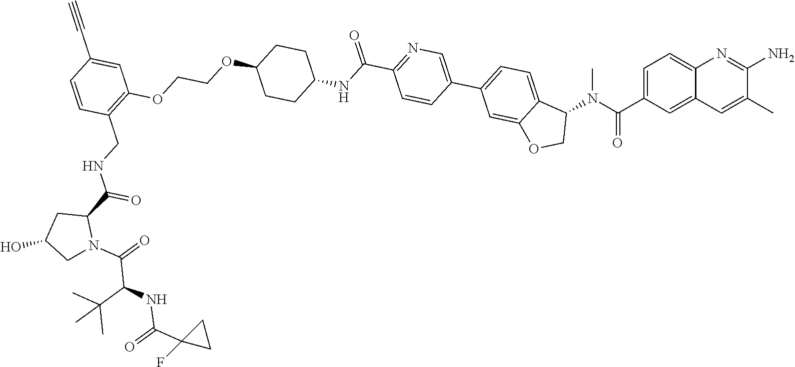 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C)cc5c4)cn3)CC2)c1] |
| A61 | 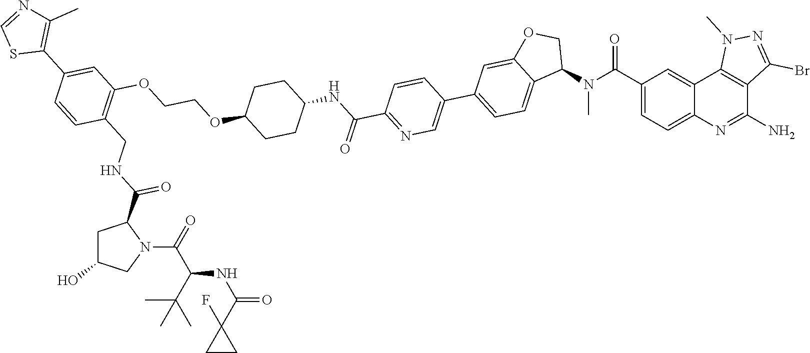 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6c(Br)nn(C)c6c5c4)cn3)CC2)c1] |
| A62 | 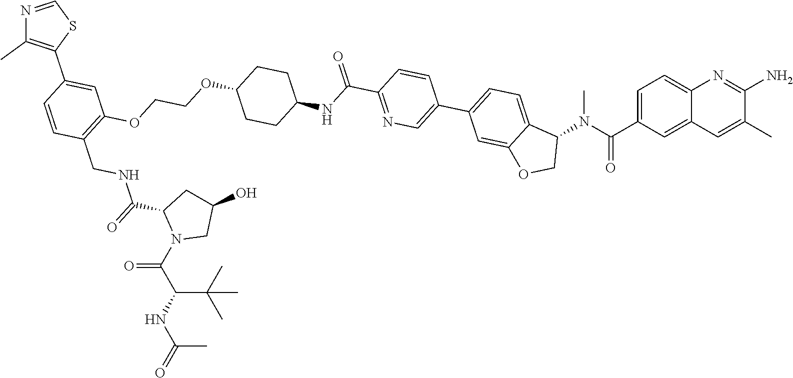 [SMILES: CC(=O)N[C@H](C(=O)N1C[C@H](O)C[C@H]1C(=O)NCc1ccc(-c2scnc2C)cc1OCCO[C@H]1CC[C@H](NC(=O)c2ccc(-c3ccc4c(c3)OC[C@H]4N(C)C(=O)c3ccc4nc(N)c(C)cc4c3)cn2)CC1)C(C)(C)C] |
| A63 | 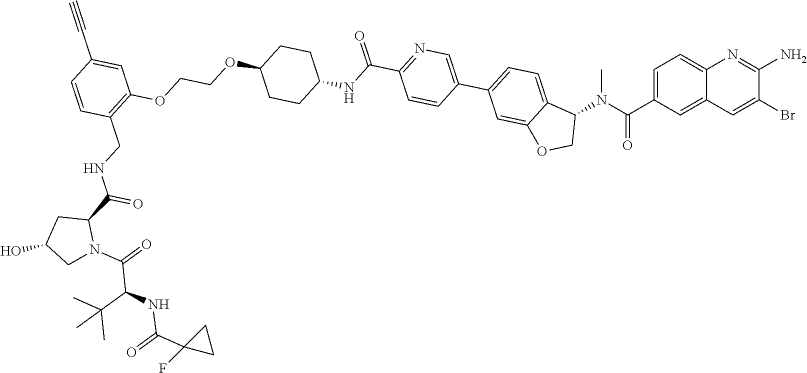 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(Br)cc5c4)cn3)CC2)c1] |
| A64 | 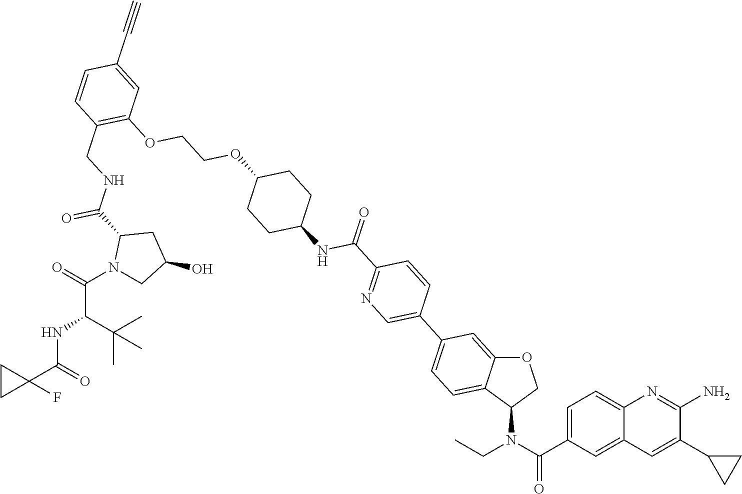 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(CC)C(=O)c4ccc5nc(N)c(C6CC6)cc5c4)cn3)CC2)c1] |
| A65 | 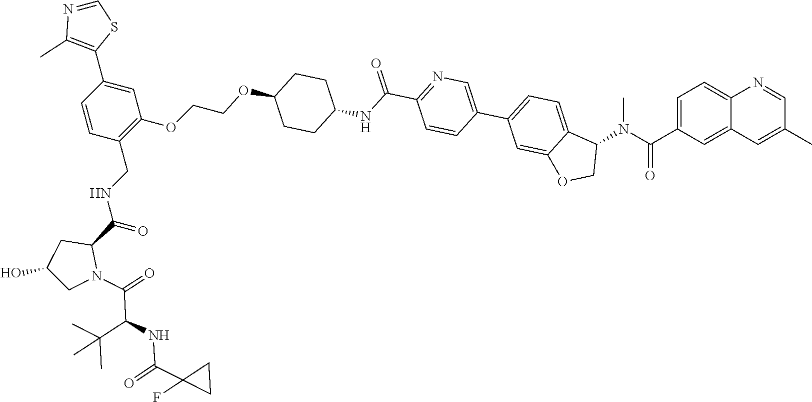 [SMILES: Cc1cnc2ccc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2c1] |
| A66 | 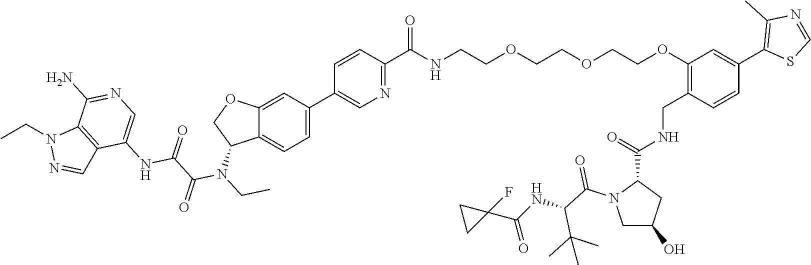 [SMILES: CCN(C(=O)C(=O)Nc1cnc(N)c2c1cnn2CC)[C@@H]1COc2cc(-c3ccc(C(=O)NCCOCCOCCOc4cc(-c5scnc5C)ccc4CNC(=O)[C@@H]4C[C@@H](O)CN4C(=O)[C@@H](NC(=O)C4(F)CC4)C(C)(C)C)nc3)ccc21] |
| A67 | 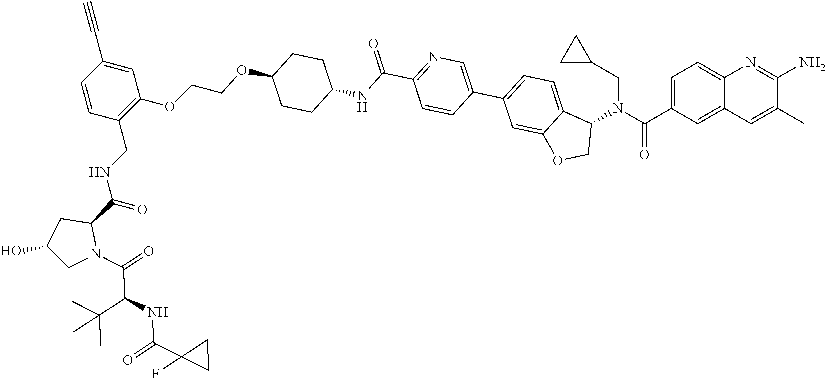 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(CC4CC4)C(=O)c4ccc5nc(N)c(C)cc5c4)cn3)CC2)c1] |
| A68 | 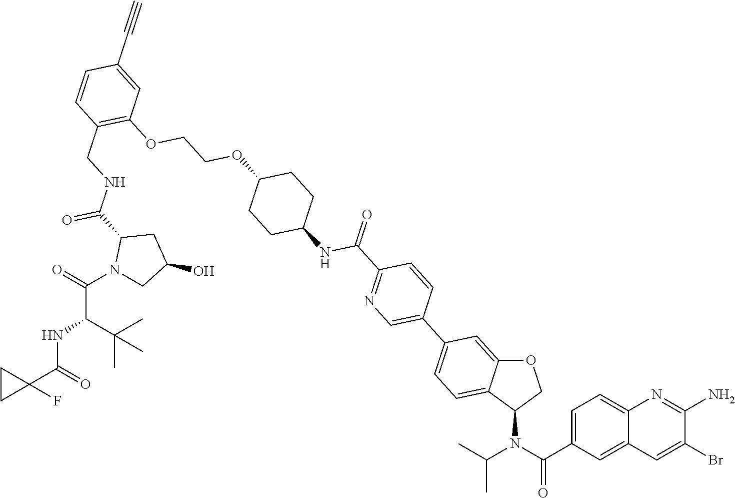 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C(=O)c4ccc5nc(N)c(Br)cc5c4)C(C)C)cn3)CC2)c1] |
| A69 | 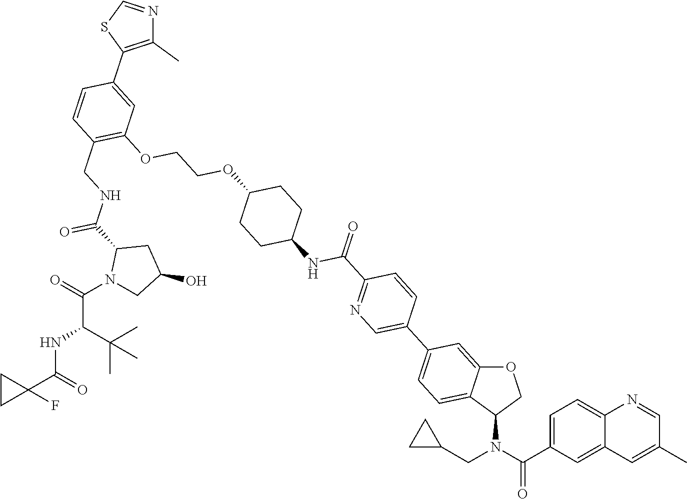 [SMILES: Cc1cnc2ccc(C(=O)N(CC3CC3)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2c1] |
| A70 | 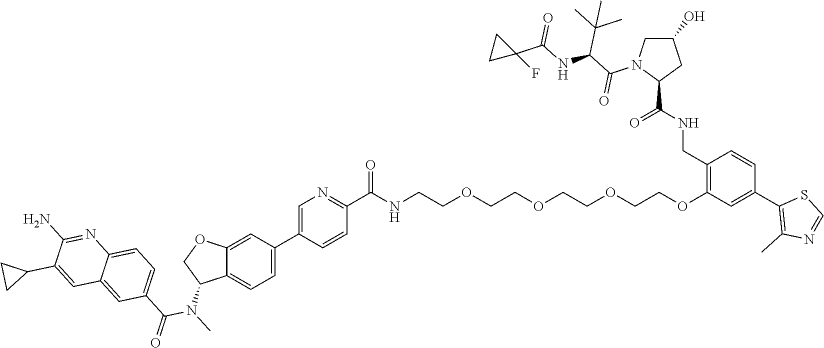 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3ccc4nc(N)c(C5CC5)cc4c3)cn2)c1] |
| A71 | 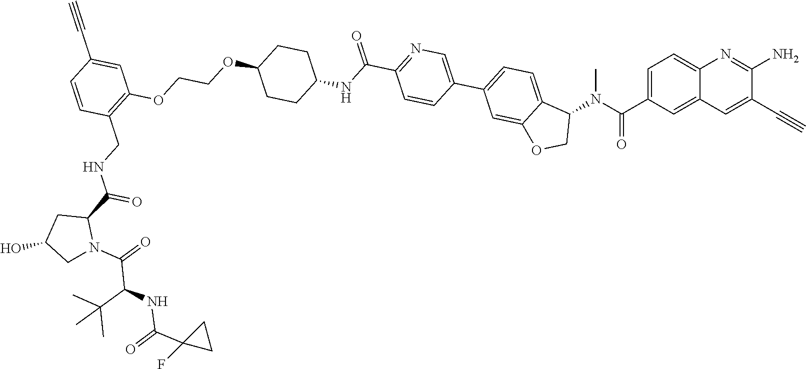 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C#C)cc5c4)cn3)CC2)c1] |
| A72 | 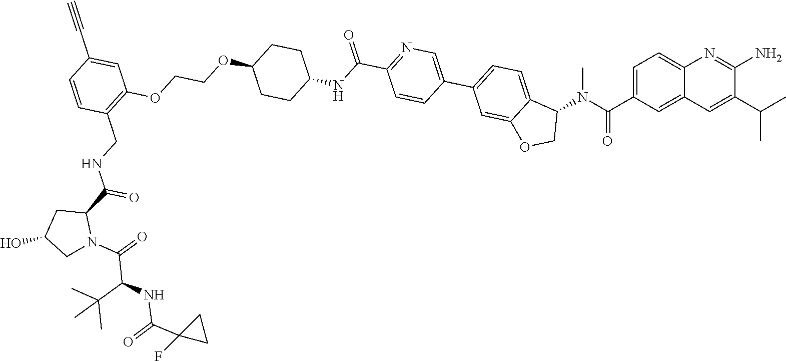 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C(C)C)cc5c4)cn3)CC2)c1] |
| A73 | 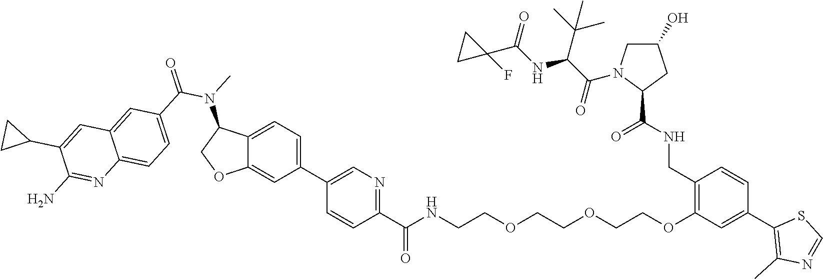 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c(C5CC5)cc4c3)cn2)c1] |
| A74 | 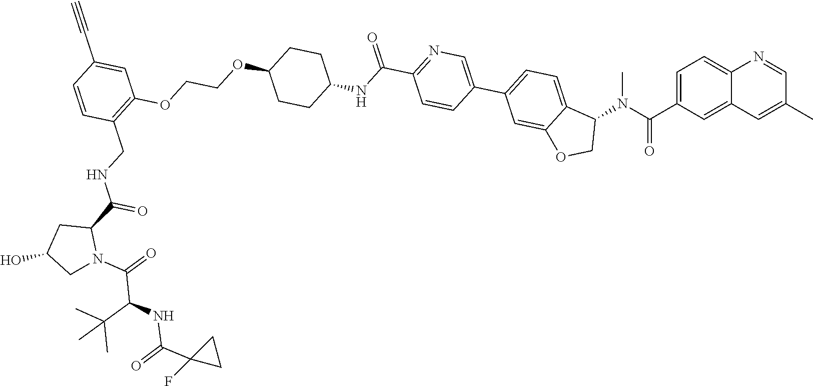 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5ncc(C)cc5c4)cn3)CC2)c1] |
| A75 | 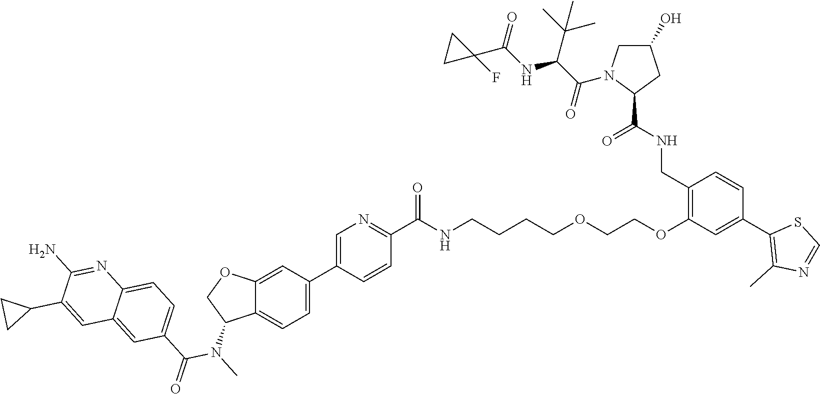 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(C5CC5)cc4c3)cn2)c1] |
| A76 | 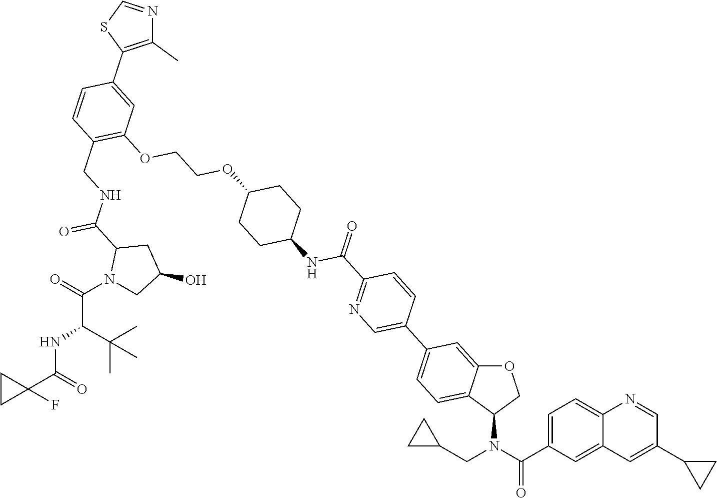 [SMILES: Cc1ncsc1-c1ccc(CNC(=O)C2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(CC4CC4)C(=O)c4ccc5ncc(C6CC6)cc5c4)cn3)CC2)c1] |
| A77 | 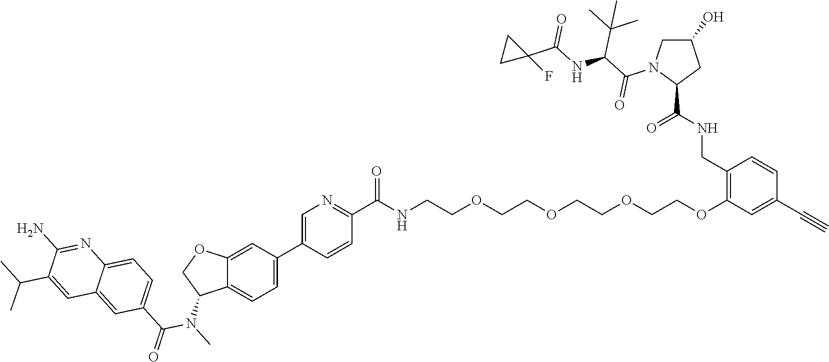 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3ccc4nc(N)c(C(C)C)cc4c3)cn2)c1] |
| A78 | 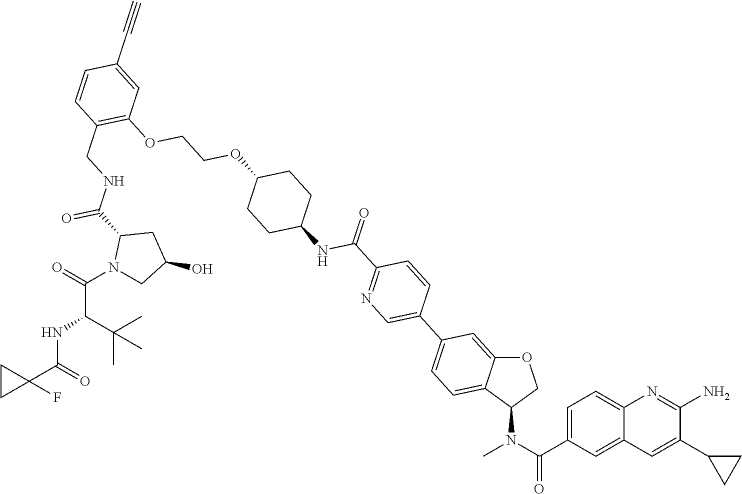 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CC6)cc5c4)cn3)CC2)c1] |
| A79 | 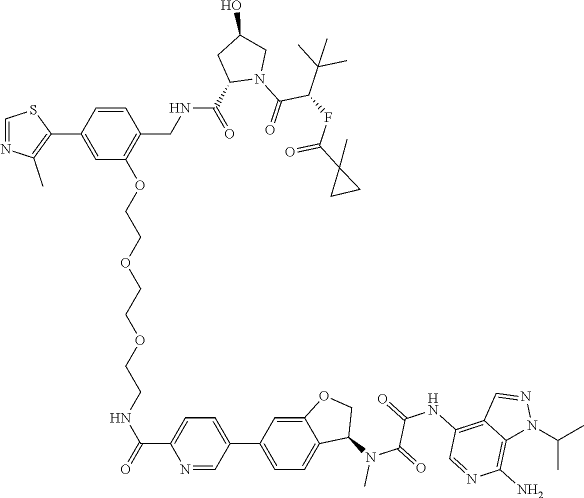 |
| A80 | 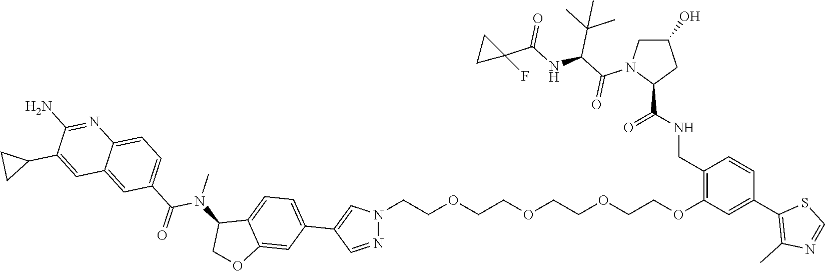 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCn2cc(-c3ccc4c(c3)OC[C@H]4N(C)C(=O)c3ccc4nc(N)c(C5CC5)cc4c3)cn2)c1] |
| A81 | 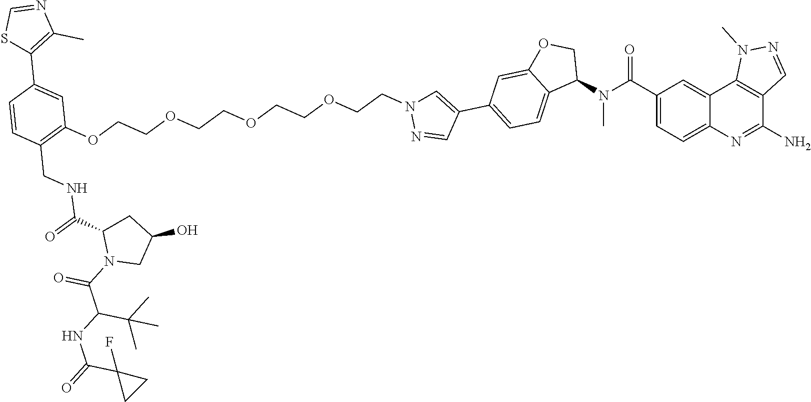 [SMILES: Cc1ncsc1-c1ccc(CNC(=O)[C@@H]2C[C@@H](O)CN2C(=O)C(NC(=O)C2(F)CC2)C(C)(C)C)c(OCCOCCOCCOCCn2cc(-c3ccc4c(c3)OC[C@H]4N(C)C(=O)c3ccc4nc(N)c5cnn(C)c5c4c3)cn2)c1] |
| A82 | 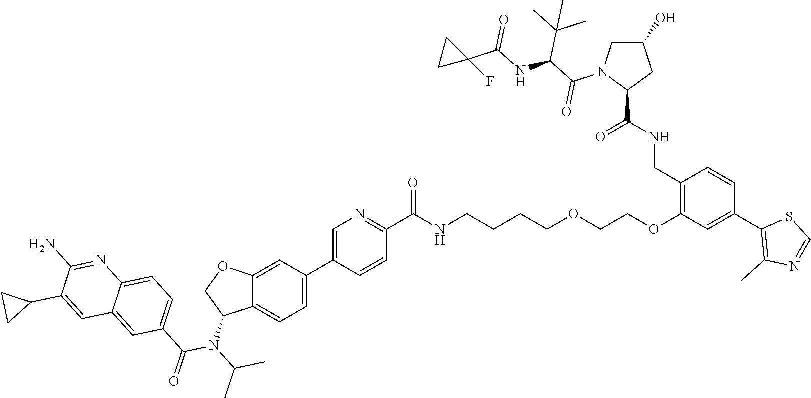 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C(=O)c3ccc4nc(N)c(C5CC5)cc4c3)C(C)C)cn2)c1] |
| A83 | 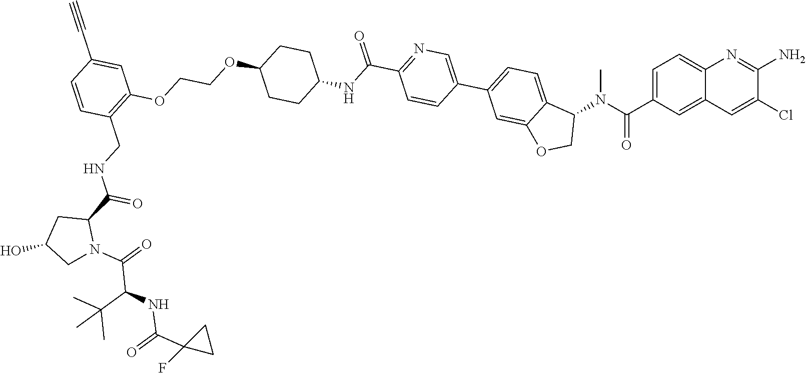 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(Cl)cc5c4)cn3)CC2)c1] |
| A84 | 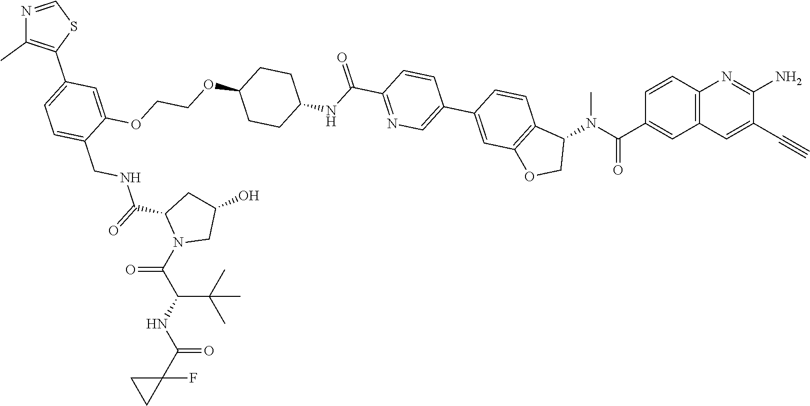 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@H](O)CN7C(=O)[C@@H](NC(=O)C7(F)CC7)C(C)(C)C)CC6)nc5)ccc43)ccc2nc1N] |
| A85 | 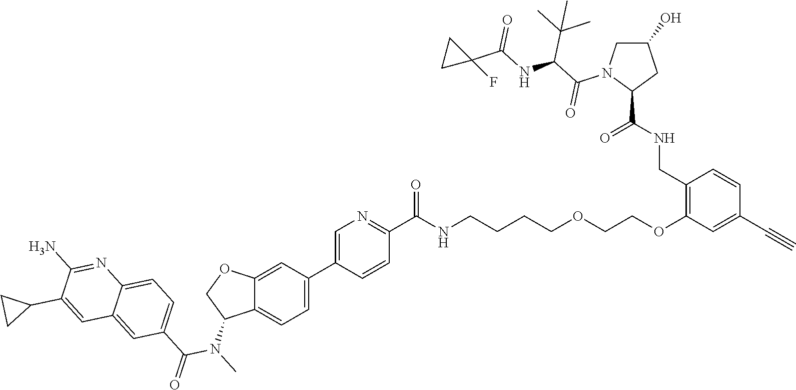 [SMILES: C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(C5CC5)cc4c3)cn2)c1] |
| A86 | 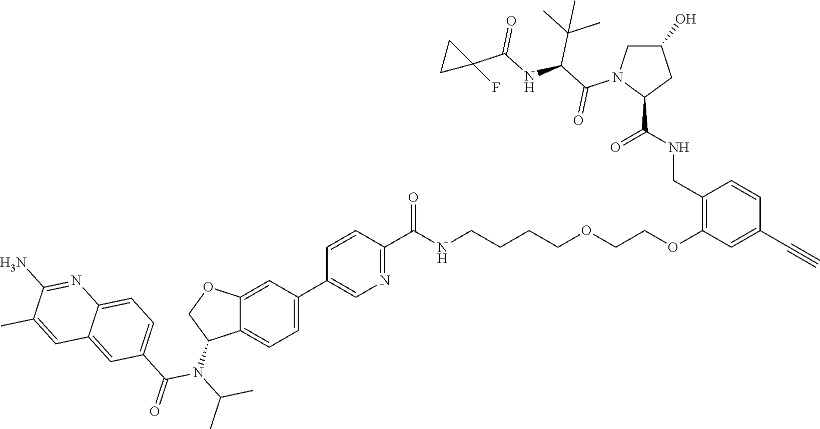 [SMILES: C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C(=O)c3ccc4nc(N)c(C)cc4c3)C(C)C)cn2)c1] |
| A87 | 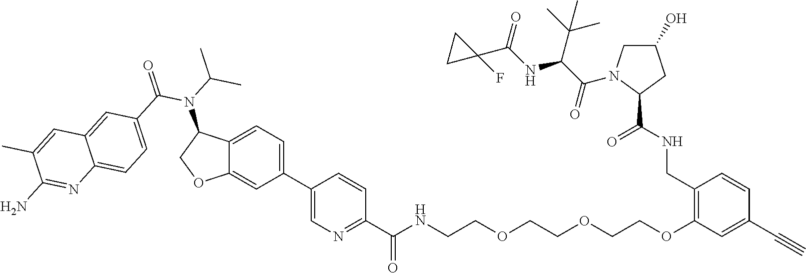 [SMILES: C#Cc1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C(=O)c3ccc4nc(N)c(C)cc4c3)C(C)C)cn2)c1] |
| A88 | 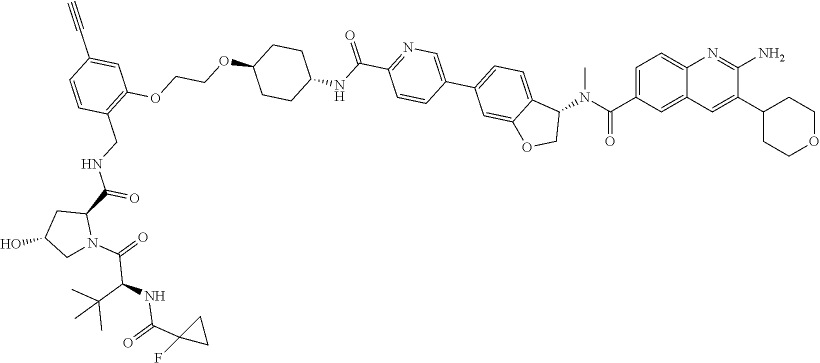 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CCOCC6)cc5c4)cn3)CC2)c1] |
| A89 | 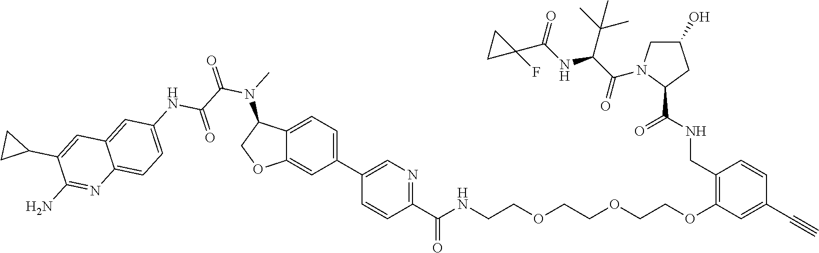 [SMILES: C#Cc1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)C(=O)Nc3ccc4nc(N)c(C5CC5)cc4c3)cn2)c1] |
| A90 | 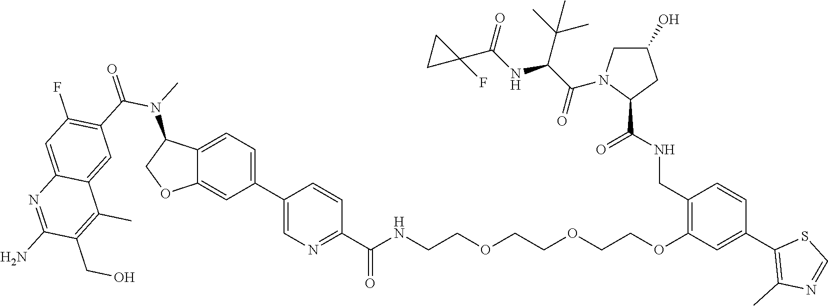 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3cc4c(C)c(CO)c(N)nc4cc3F)cn2)c1] |
| A91 | 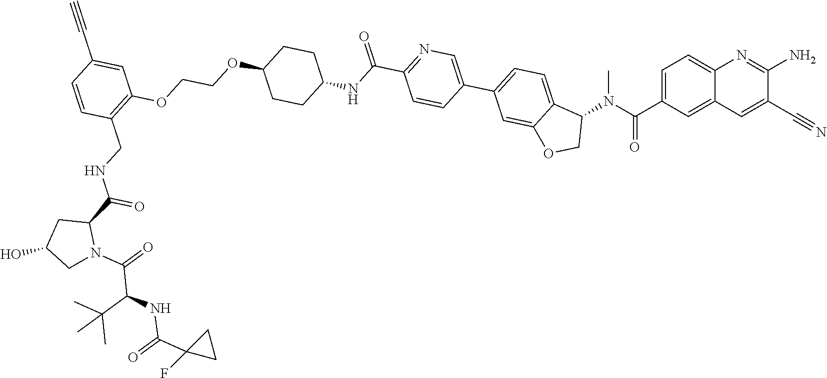 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A92 | 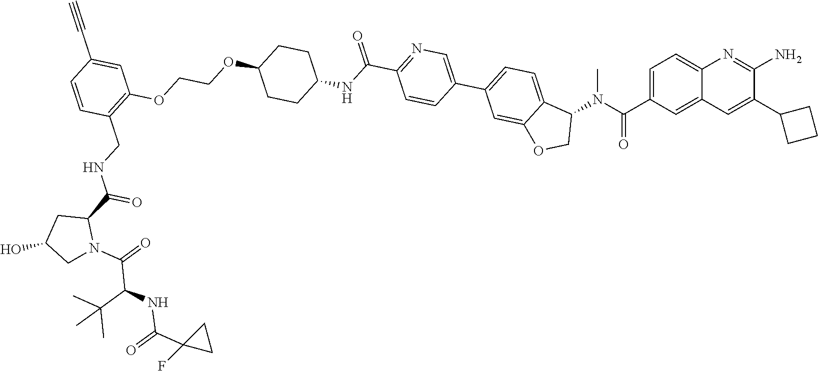 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CCC6)cc5c4)cn3)CC2)c1] |
| A93 | 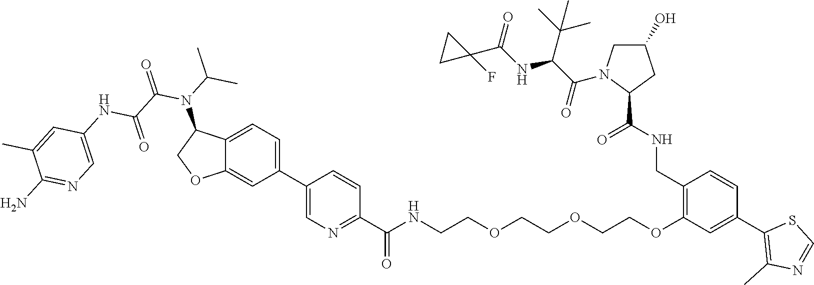 [SMILES: Cc1cc(NC(=O)C(=O)N(C(C)C)[C@@H]2COc3cc(-c4ccc(C(=O)NCCOCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A94 | 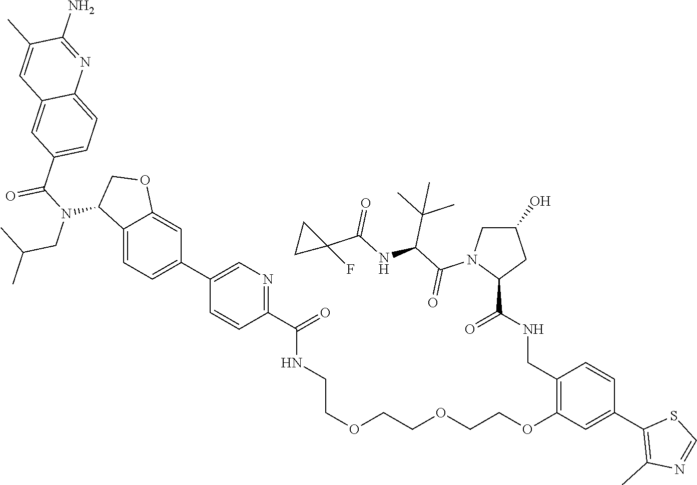 [SMILES: Cc1cc2cc(C(=O)N(CC(C)C)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A95 | 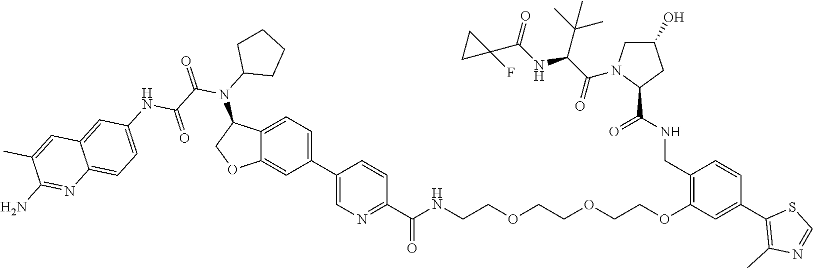 [SMILES: Cc1cc2cc(NC(=O)C(=O)N(C3CCCC3)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A96 | 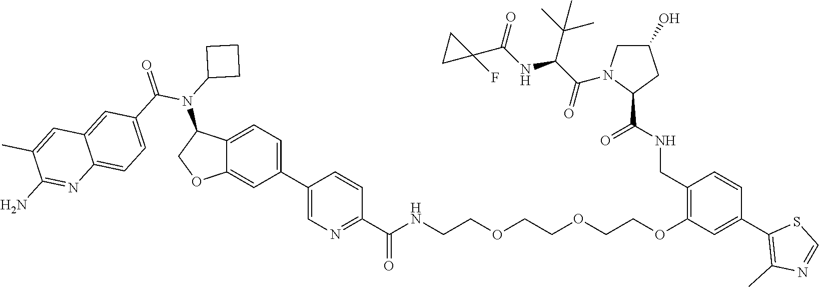 [SMILES: Cc1cc2cc(C(=O)N(C3CCC3)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A97 | 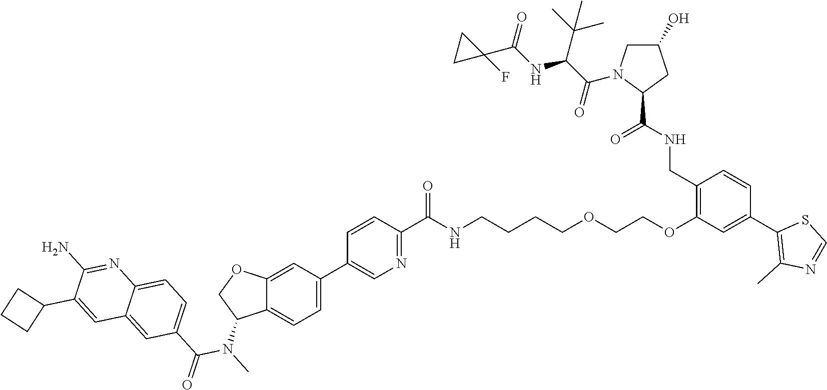 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(C5CCC5)cc4c3)cn2)c1] |
| A98 | 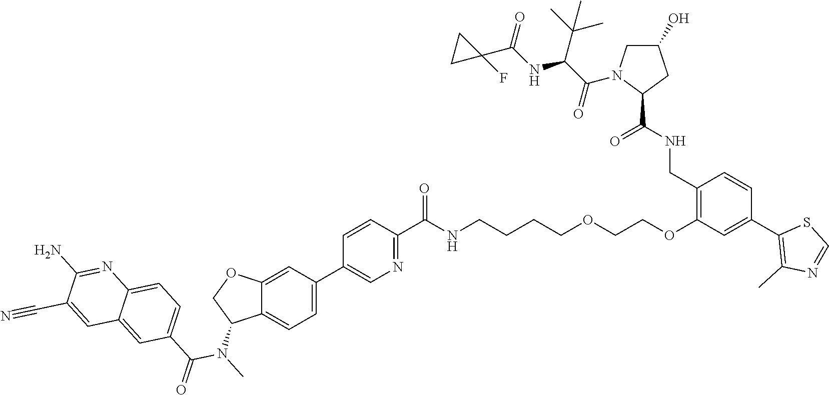 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(C#N)cc4c3)cn2)c1] |
| A99 | 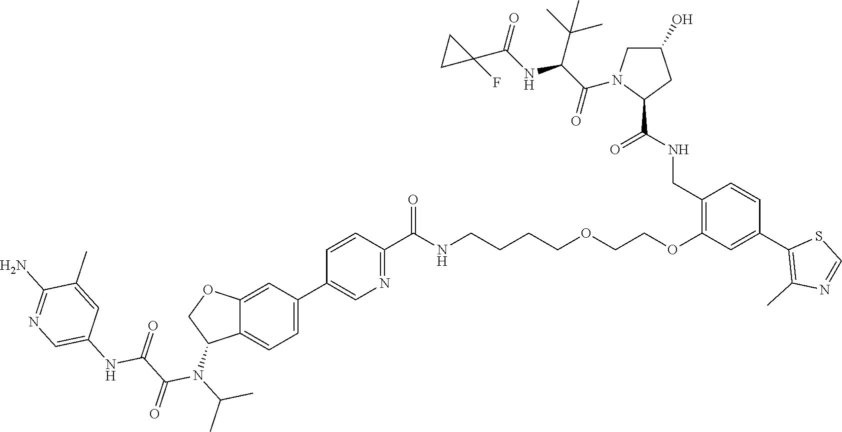 [SMILES: Cc1cc(NC(=O)C(=O)N(C(C)C)[C@@H]2COc3cc(-c4ccc(C(=O)NCCCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A100 | 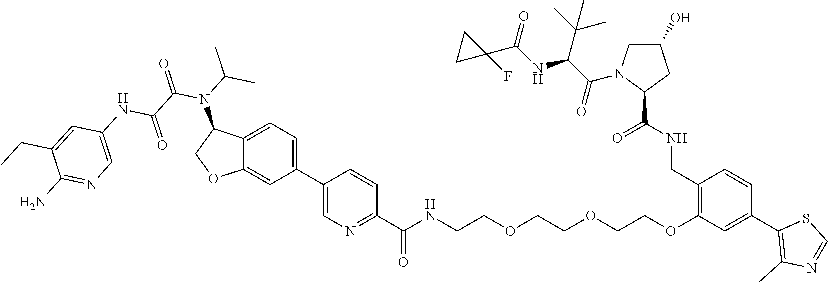 [SMILES: CCc1cc(NC(=O)C(=O)N(C(C)C)[C@@H]2COc3cc(-c4ccc(C(=O)NCCOCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A101 | 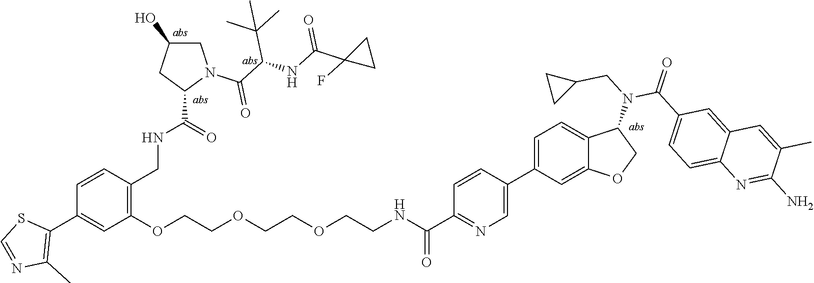 [SMILES: Cc1cc2cc(C(=O)N(CC3CC3)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A102 | 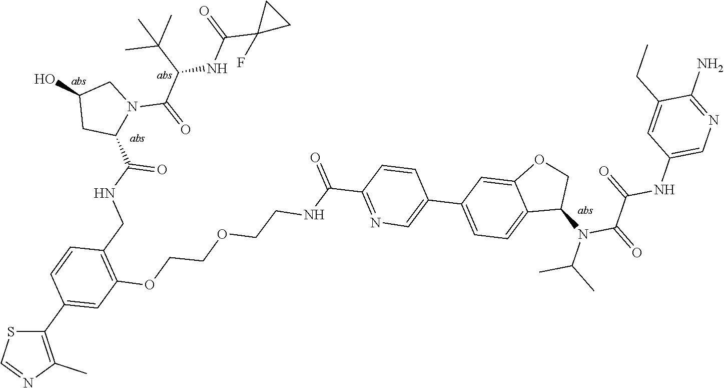 [SMILES: CCc1cc(NC(=O)C(=O)N(C(C)C)[C@@H]2COc3cc(-c4ccc(C(=O)NCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A103 | 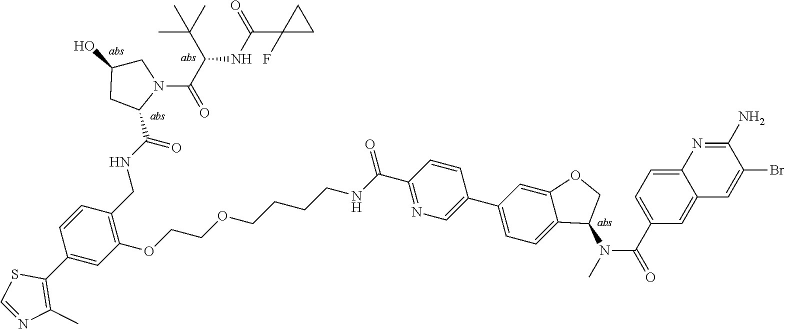 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(Br)cc4c3)cn2)c1] |
| A104 | 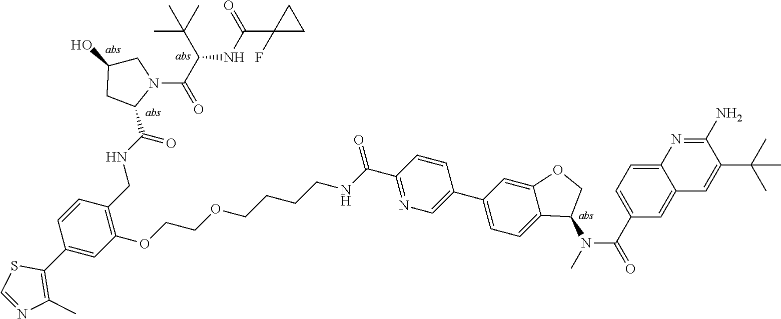 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(C(C)(C)C)cc4c3)cn2)c1] |
| A105 | 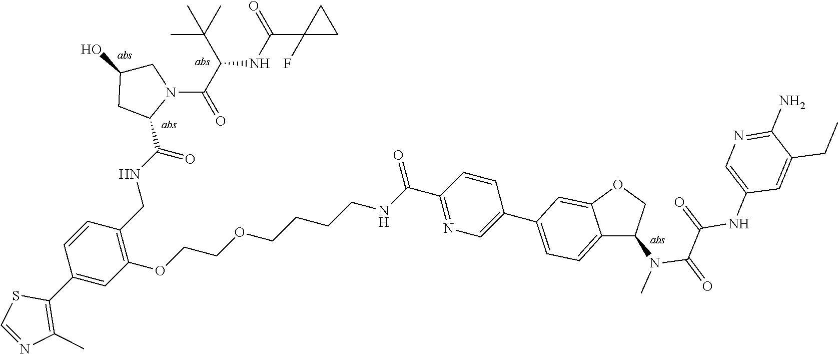 [SMILES: CCc1cc(NC(=O)C(=O)N(C)[C@@H]2COc3cc(-c4ccc(C(=O)NCCCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N] |
| A106 | 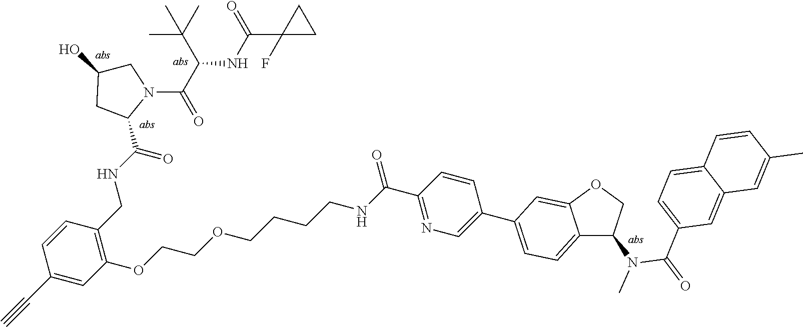 [SMILES: C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4ccc(C)cc4c3)cn2)c1] |
| A107 | 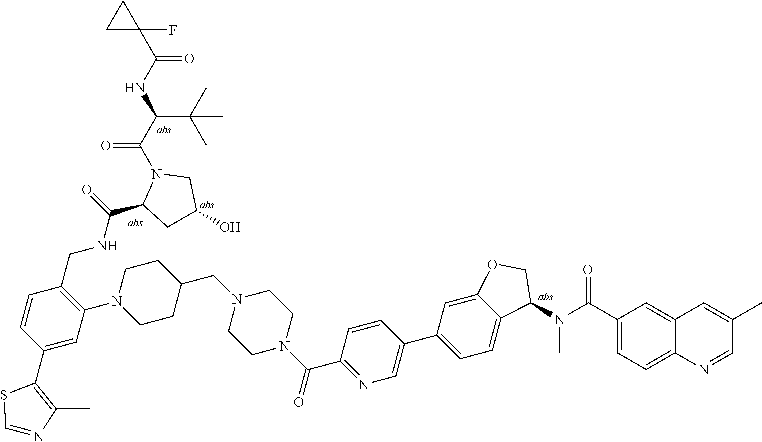 [SMILES: Cc1cnc2ccc(C(=O)N(C)[C@@H]3COc4cc(-c5ccc(C(=O)N6CCN(CC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)cc2c1] |
| A108 | 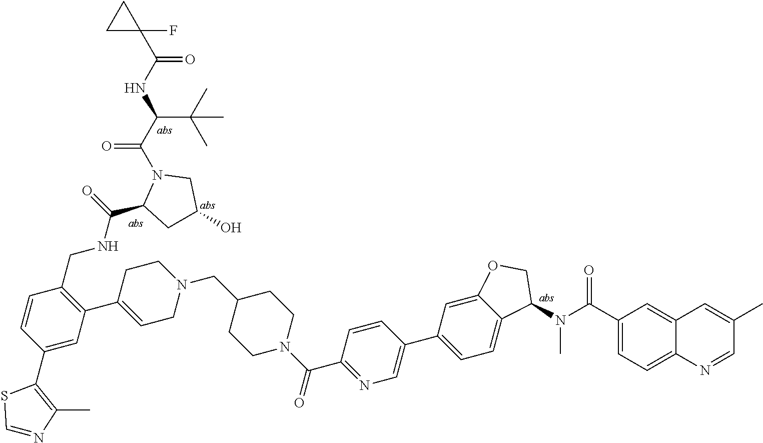 [SMILES: Cc1cnc2ccc(C(=O)N(C)[C@@H]3COc4cc(-c5ccc(C(=O)N6CCC(CN7CC=C(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)cc2c1] |
| A109 | 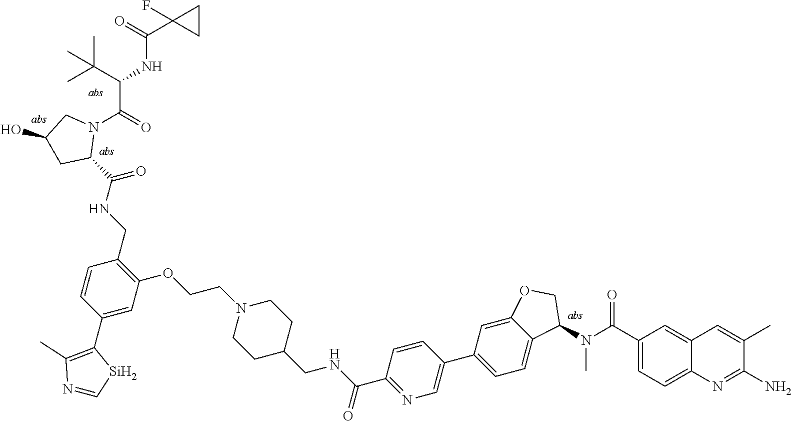 |
| A110 | 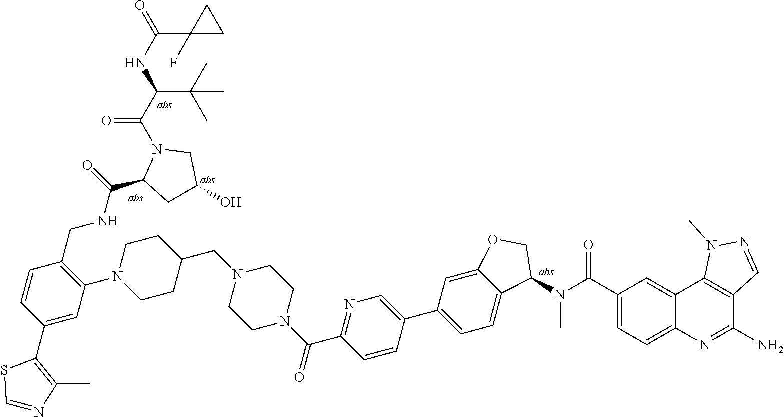 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7cnn(C)c7c6c5)cn4)CC3)CC2)c1] |
| A111 | 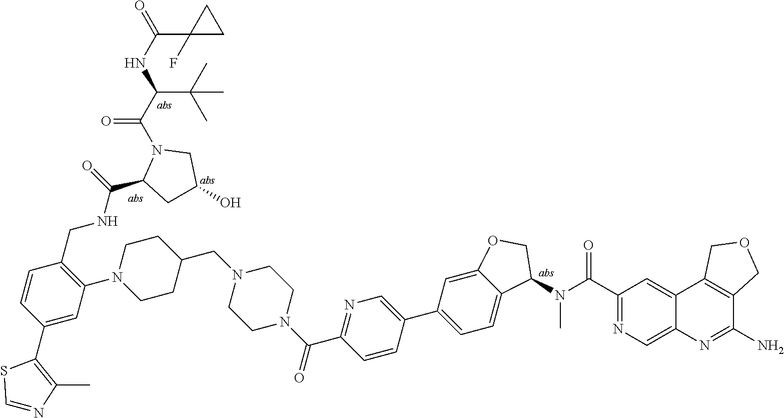 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5cc6c7c(c(N)nc6cn5)COC7)cn4)CC3)CC2)c1] |
| A112 | 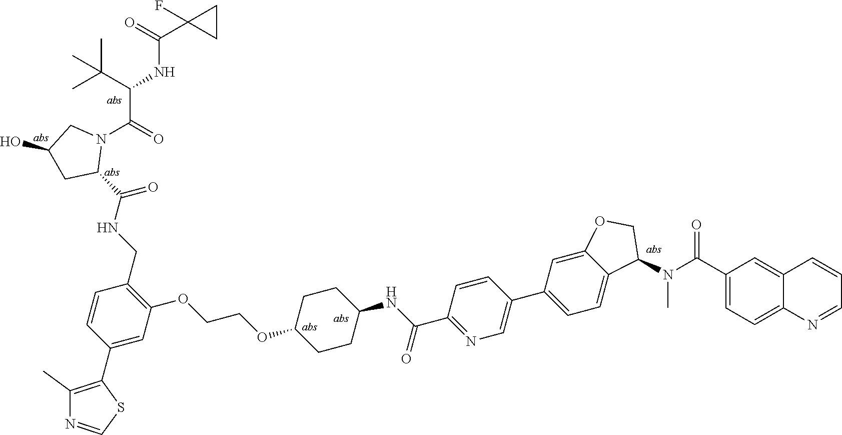 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5ncccc5c4)cn3)CC2)c1] |
| A113 | 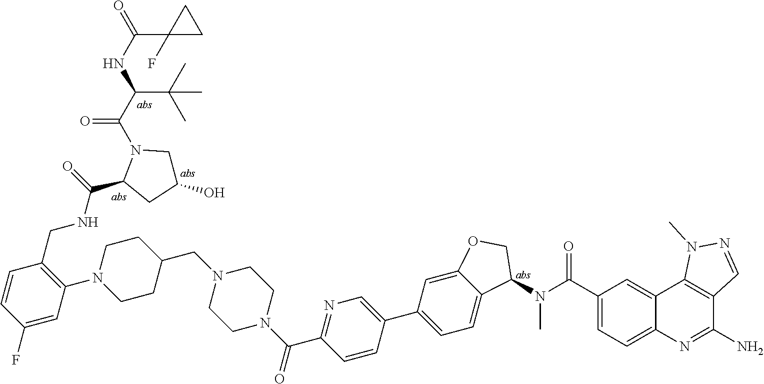 [SMILES: CN(C(=O)c1ccc2nc(N)c3cnn(C)c3c2c1)[C@@H]1COc2cc(-c3ccc(C(=O)N4CCN(CC5CCN(c6cc(F)ccc6CNC(=O)[C@@H]6C[C@@H](O)CN6C(=O)[C@@H](NC(=O)C6(F)CC6)C(C)(C)C)CC5)CC4)nc3)ccc21] |
| A114 | 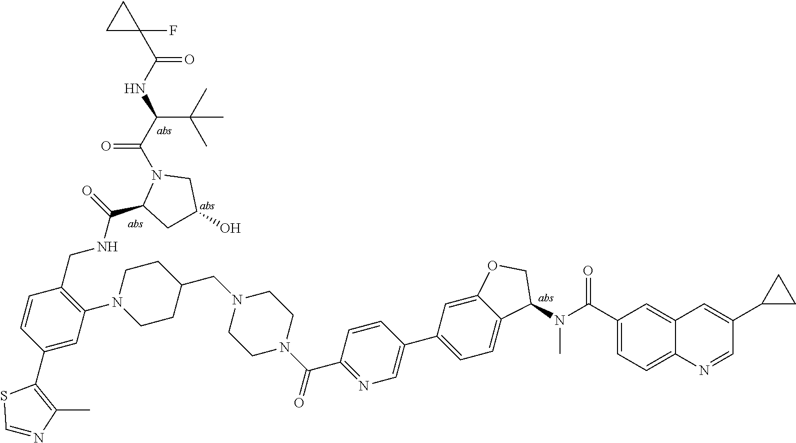 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncc(C7CC7)cc6c5)cn4)CC3)CC2)c1] |
| A115 | 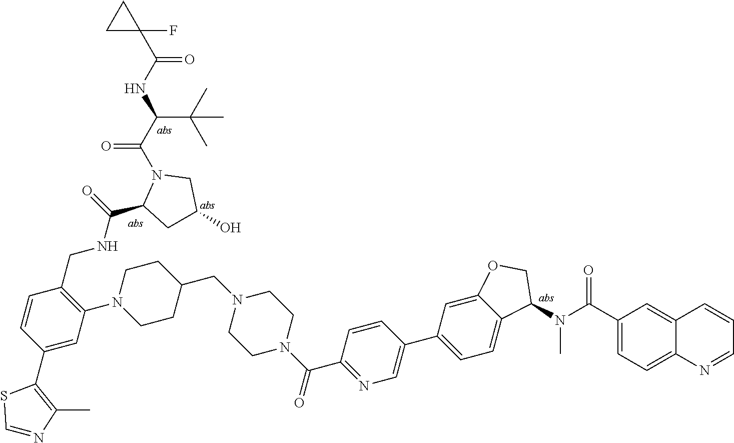 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncccc6c5)cn4)CC3)CC2)c1] |
| A116 | 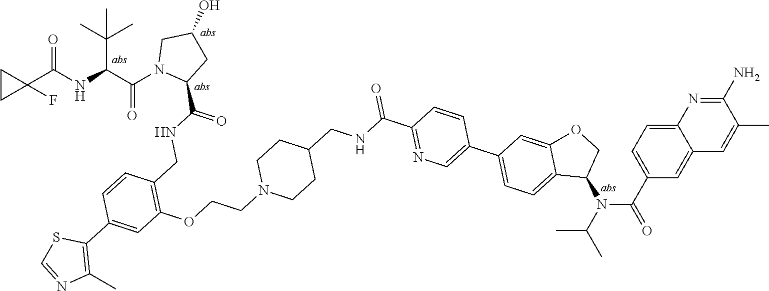 [SMILES: Cc1cc2cc(C(=O)N(C(C)C)[C@@H]3COc4cc(-c5ccc(C(=O)NCC6CCN(CCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A117 | 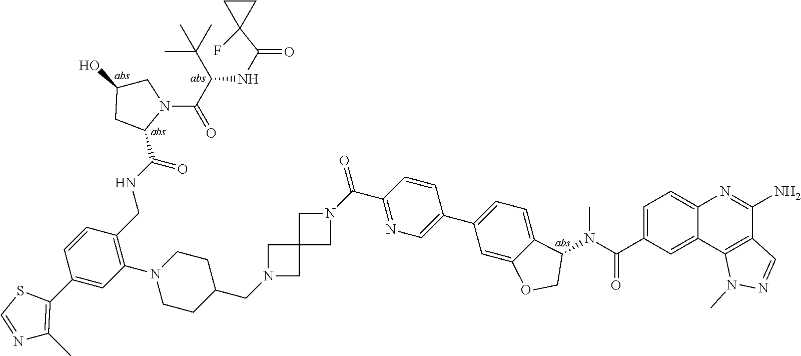 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4(C3)CN(C(=O)c3ccc(-c5ccc6c(c5)OC[C@H]6N(C)C(=O)c5ccc6nc(N)c7cnn(C)c7c6c5)cn3)C4)CC2)c1] |
| A118 | 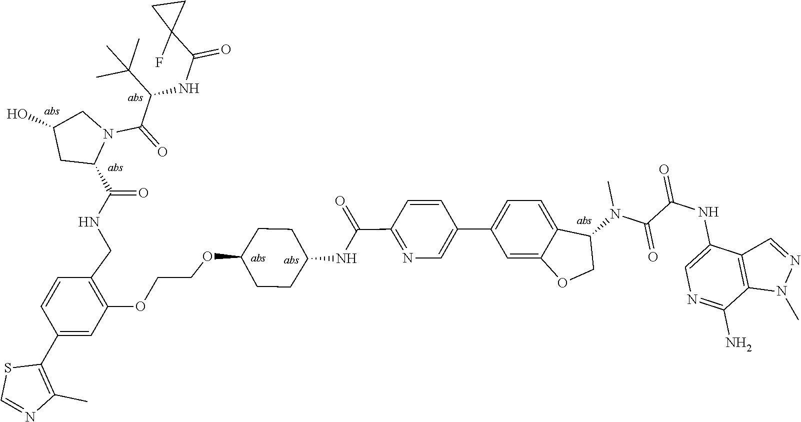 [SMILES: Cc1ncsc1-c1ccc(CNC(=O)[C@@H]2C[C@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)C(=O)Nc4cnc(N)c5c4cnn5C)cn3)CC2)c1] |
| A119 | 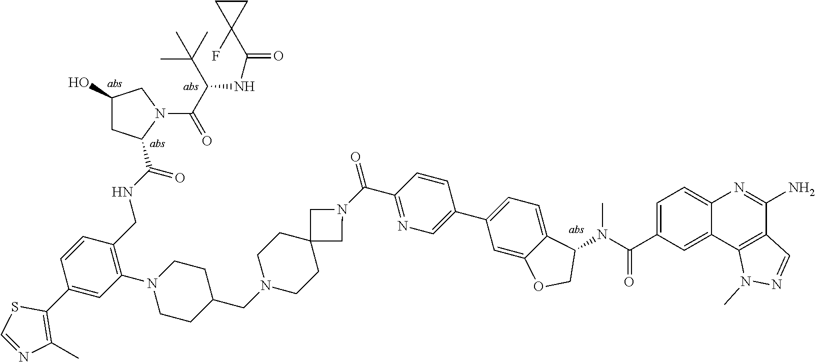 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCC4(CC3)CN(C(=O)c3ccc(-c5ccc6c(c5)OC[C@H]6N(C)C(=O)c5ccc6nc(N)c7cnn(C)c7c6c5)cn3)C4)CC2)c1] |
| A120 | 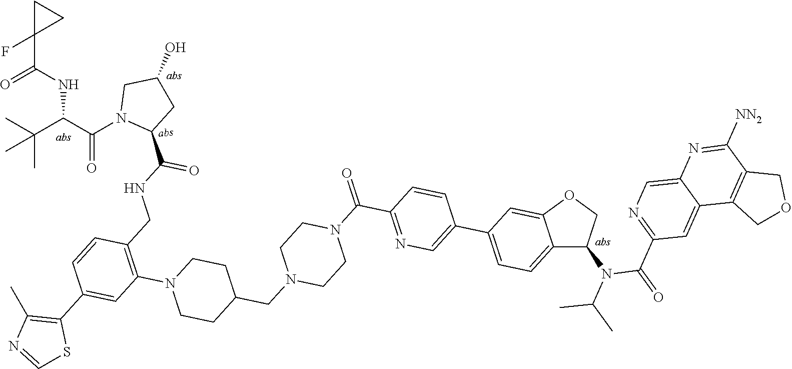 |
| A121 | 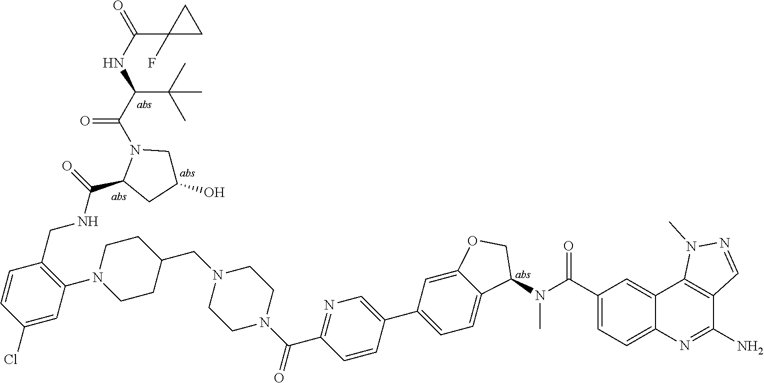 [SMILES: CN(C(=O)c1ccc2nc(N)c3cnn(C)c3c2c1)[C@@H]1COc2cc(-c3ccc(C(=O)N4CCN(CC5CCN(c6cc(Cl)ccc6CNC(=O)[C@@H]6C[C@@H](O)CN6C(=O)[C@@H](NC(=O)C6(F)CC6)C(C)(C)C)CC5)CC4)nc3)ccc21] |
| A122 | 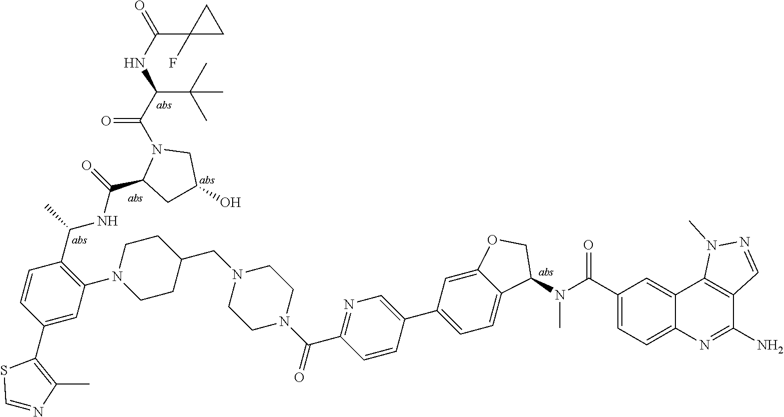 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7cnn(C)c7c6c5)cn4)CC3)CC2)c1] |
| A123 | 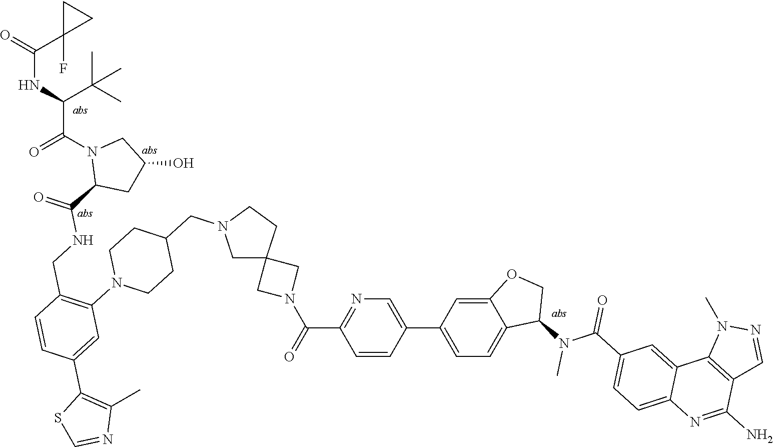 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCC4(C3)CN(C(=O)c3ccc(-c5ccc6c(c5)OC[C@H]6N(C)C(=O)c5ccc6nc(N)c7cnn(C)c7c6c5)cn3)C4)CC2)c1] |
| A124 | 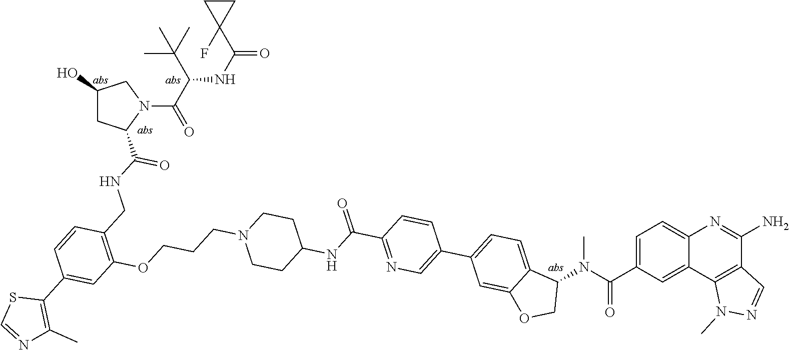 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCN2CCC(NC(=O)c3ccc(-c4ccc5c(c4)OC[C@H]5N(C)C(=O)c4ccc5nc(N)c6cnn(C)c6c5c4)cn3)CC2)c1] |
| A125 | 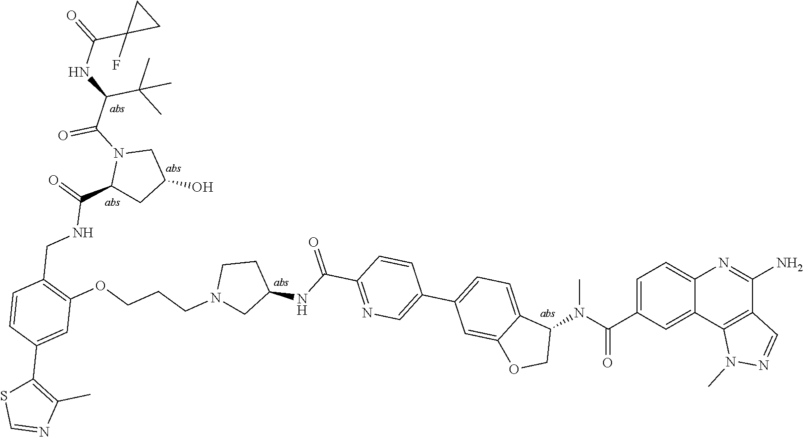 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCN2CC[C@@H](NC(=O)c3ccc(-c4ccc5c(c4)OC[C@H]5N(C)C(=O)c4ccc5nc(N)c6cnn(C)c6c5c4)cn3)C2)c1] |
| A126 | 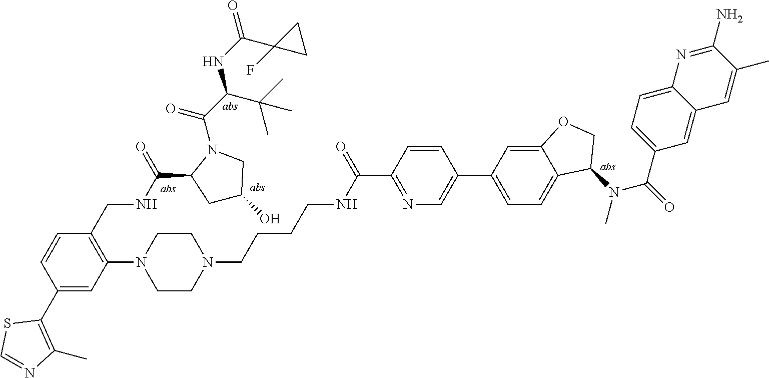 [SMILES: Cc1cc2cc(C(=O)N(C)[C@@H]3COc4cc(-c5ccc(C(=O)NCCCCN6CCN(c7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A127 | 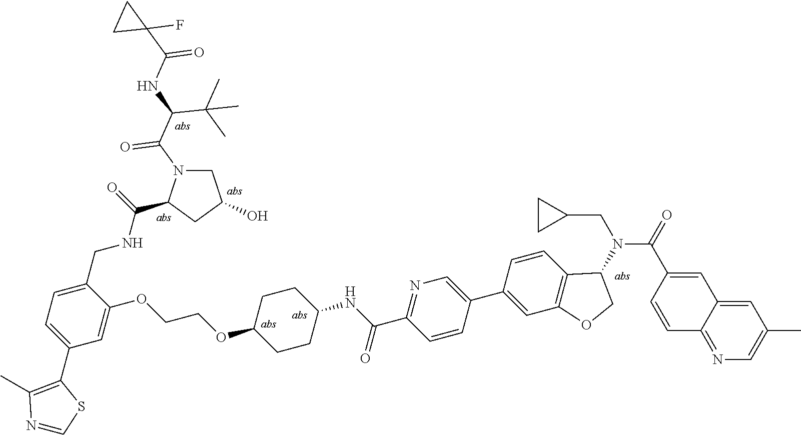 |
| A128 | 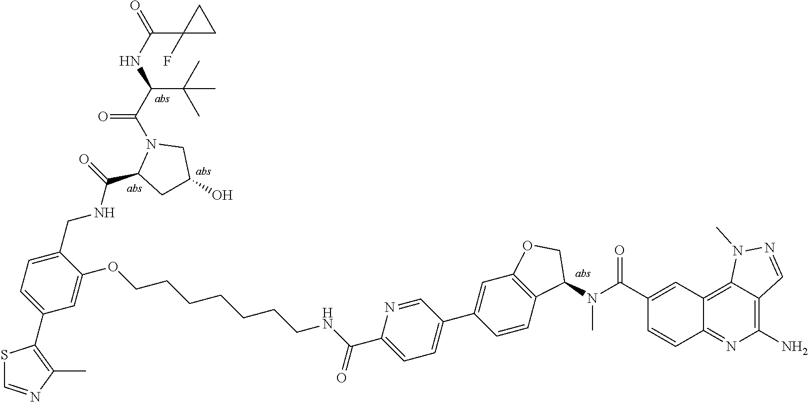 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCNC(=O)c2ccc(-c3ccc4c(c3)OC[C@H]4N(C)C(=O)c3ccc4nc(N)c5cnn(C)c5c4c3)cn2)c1] |
| A129 | 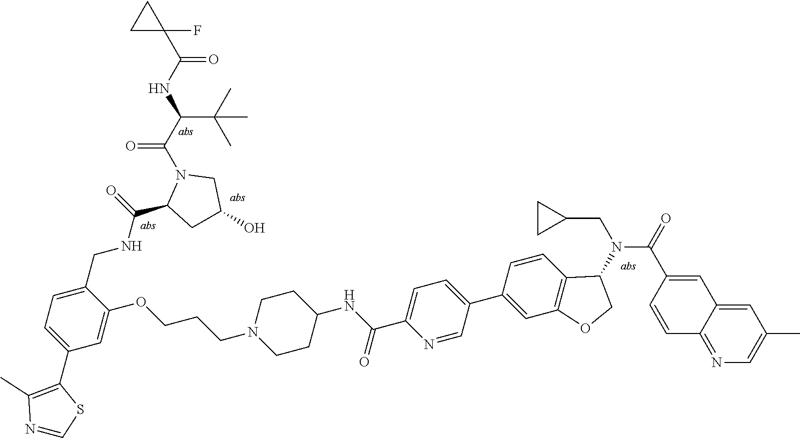 |
| A130 | 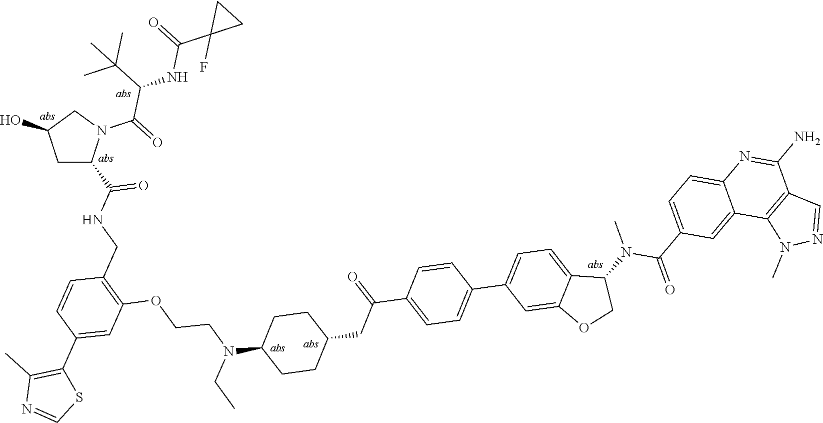 [SMILES: CCN(CCOc1cc(-c2scnc2C)ccc1CNC(=O)[C@@H]1C[C@@H](O)CN1C(=O)[C@@H](NC(=O)C1(F)CC1)C(C)(C)C)[C@H]1CC[C@H](CC(=O)c2ccc(-c3ccc4c(c3)OC[C@H]4N(C)C(=O)c3ccc4nc(N)c5cnn(C)c5c4c3)cc2)CC1] |
| A131 | 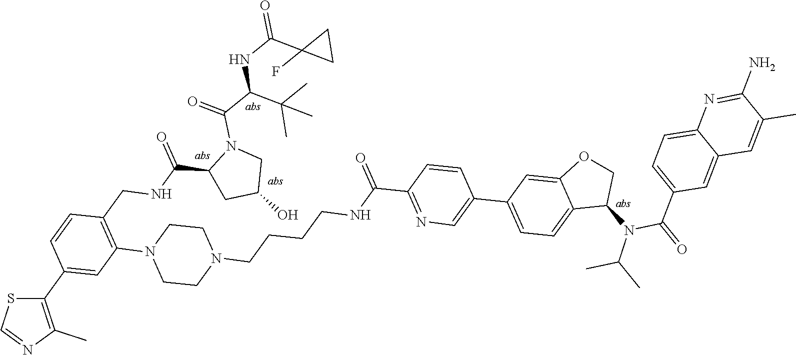 [SMILES: Cc1cc2cc(C(=O)N(C(C)C)[C@@H]3COc4cc(-c5ccc(C(=O)NCCCCN6CCN(c7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A132 | 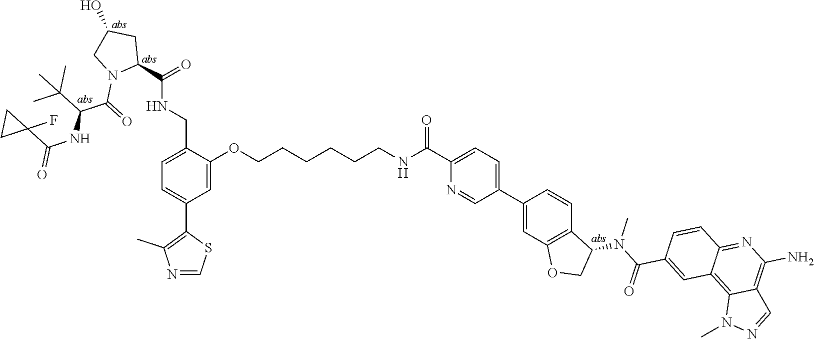 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCNC(=O)c2ccc(-c3ccc4c(c3)OC[C@H]4N(C)C(=O)c3ccc4nc(N)c5cnn(C)c5c4c3)cn2)c1] |
| A133 | 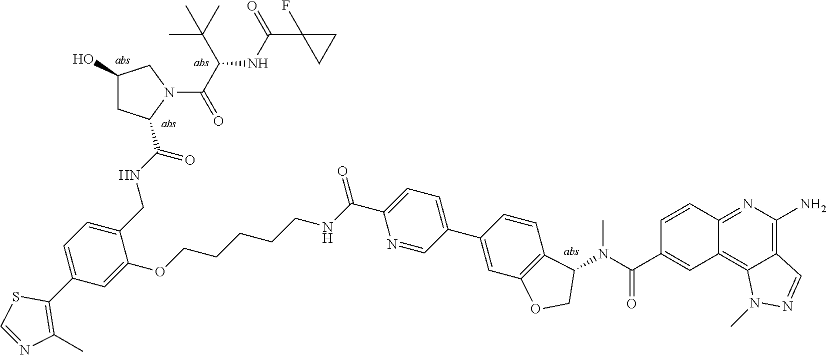 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCNC(=O)c2ccc(-c3ccc4c(c3)OC[C@H]4N(C)C(=O)c3ccc4nc(N)c5cnn(C)c5c4c3)cn2)c1] |
| A134 | 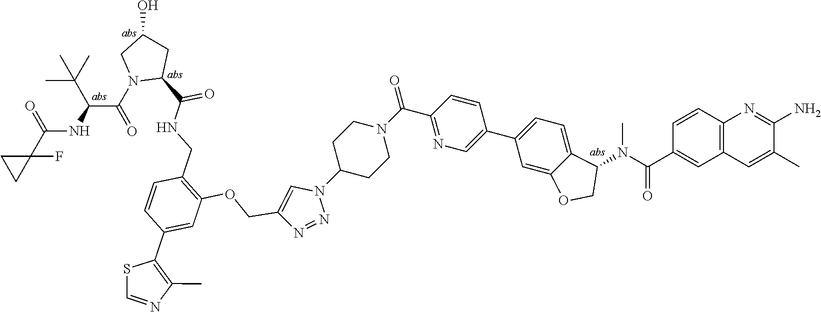 [SMILES: Cc1cc2cc(C(=O)N(C)[C@@H]3COc4cc(-c5ccc(C(=O)N6CCC(n7cc(COc8cc(-c9scnc9C)ccc8CNC(=O)[C@@H]8C[C@@H](O)CN8C(=O)[C@@H](NC(=O)C8(F)CC8)C(C)(C)C)nn7)CC6)nc5)ccc43)ccc2nc1N] |
| A135 | 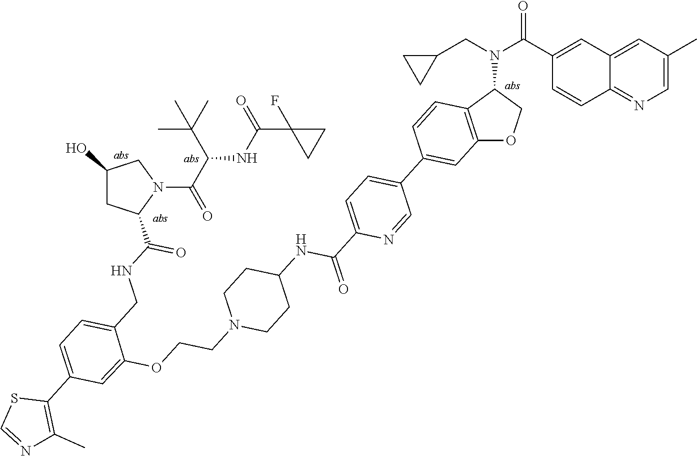 [SMILES: Cc1cnc2ccc(C(=O)N(CC3CC3)[C@@H]3COc4cc(-c5ccc(C(=O)NC6CCN(CCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2c1] |
| A136 | 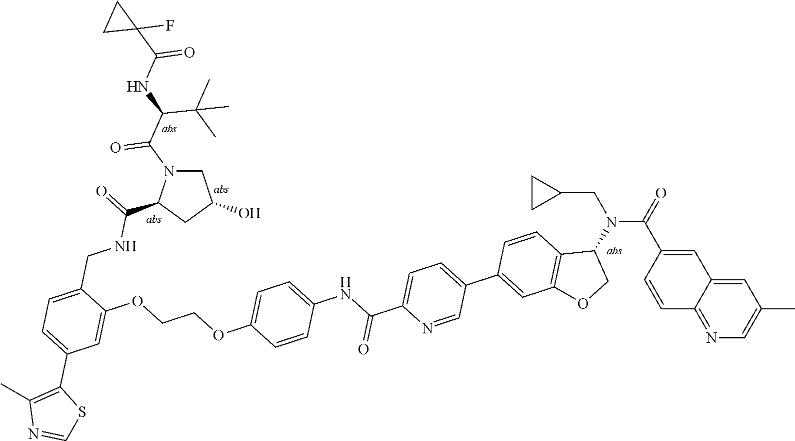 |
| A137 | 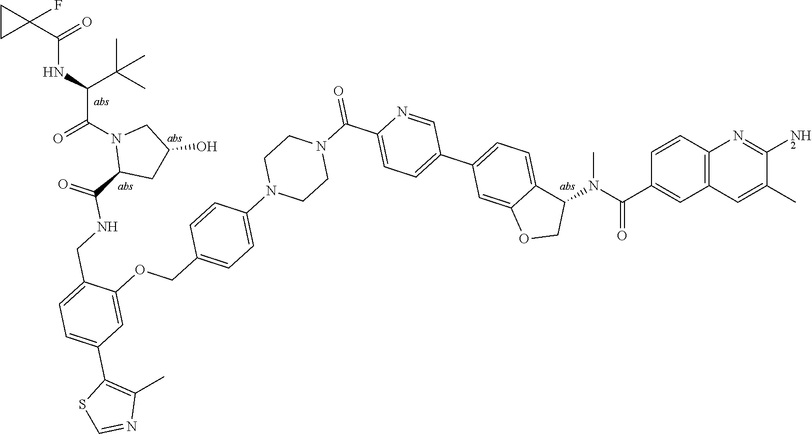 [SMILES: Cc1cc2cc(C(=O)N(C)[C@@H]3COc4cc(-c5ccc(C(=O)N6CCN(c7ccc(COc8cc(-c9scnc9C)ccc8CNC(=O)[C@@H]8C[C@@H](O)CN8C(=O)[C@@H](NC(=O)C8(F)CC8)C(C)(C)C)cc7)CC6)nc5)ccc43)ccc2nc1N] |
| A138 | 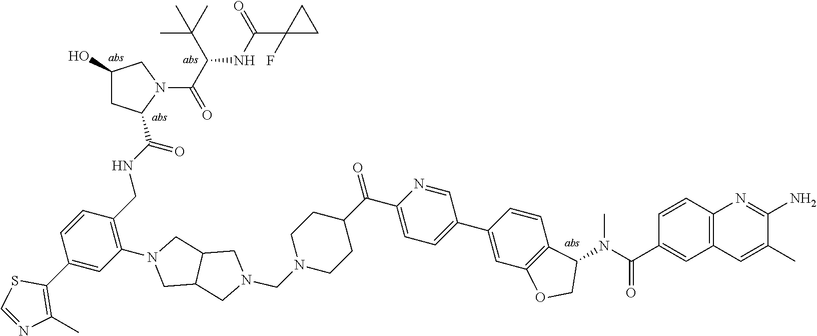 [SMILES: Cc1cc2cc(C(=O)N(C)[C@@H]3COc4cc(-c5ccc(C(=O)C6CCN(CN7CC8CN(c9cc(-c%10scnc%10C)ccc9CNC(=O)[C@@H]9C[C@@H](O)CN9C(=O)[C@@H](NC(=O)C9(F)CC9)C(C)(C)C)CC8C7)CC6)nc5)ccc43)ccc2nc1N] |
| A139 | 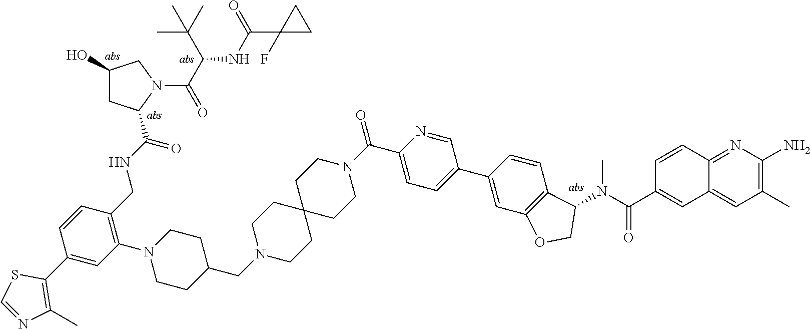 [SMILES: Cc1cc2cc(C(=O)N(C)[C@@H]3COc4cc(-c5ccc(C(=O)N6CCC7(CCN(CC8CCN(c9cc(-c%10scnc%10C)ccc9CNC(=O)[C@@H]9C[C@@H](O)CN9C(=O)[C@@H](NC(=O)C9(F)CC9)C(C)(C)C)CC8)CC7)CC6)nc5)ccc43)ccc2nc1N] |
| A140 | 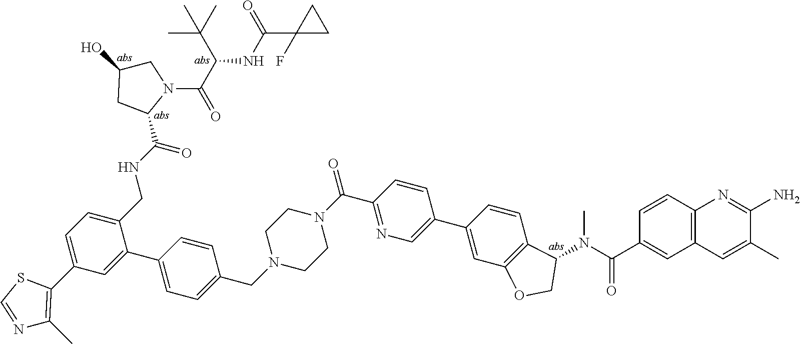 [SMILES: Cc1cc2cc(C(=O)N(C)[C@@H]3COc4cc(-c5ccc(C(=O)N6CCN(Cc7ccc(-c8cc(-c9scnc9C)ccc8CNC(=O)[C@@H]8C[C@@H](O)CN8C(=O)[C@@H](NC(=O)C8(F)CC8)C(C)(C)C)cc7)CC6)nc5)ccc43)ccc2nc1N] |
| A141 | 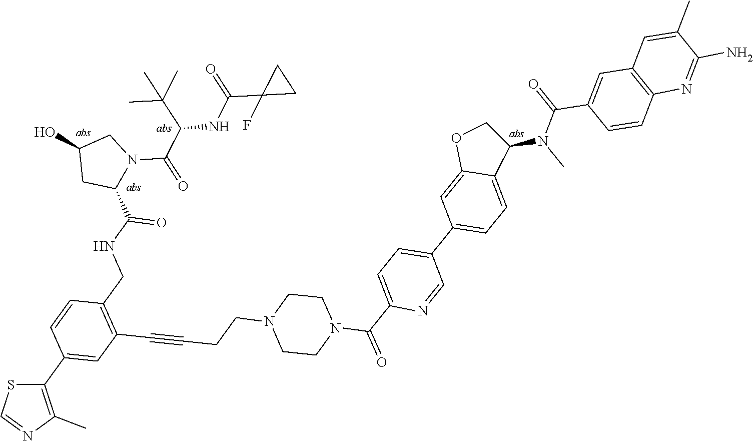 [SMILES: Cc1cc2cc(C(=O)N(C)[C@@H]3COc4cc(-c5ccc(C(=O)N6CCN(CCC#Cc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A142 | 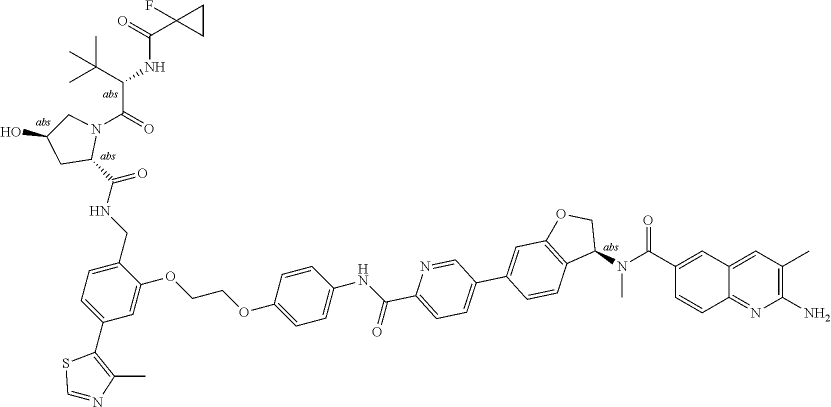 [SMILES: Cc1cc2cc(C(=O)N(C)[C@@H]3COc4cc(-c5ccc(C(=O)Nc6ccc(OCCOc7cc(-c8scnc8C)ccc7CNC(=O)[C@@H]7C[C@@H](O)CN7C(=O)[C@@H](NC(=O)C7(F)CC7)C(C)(C)C)cc6)nc5)ccc43)ccc2nc1N] |
| A143 | 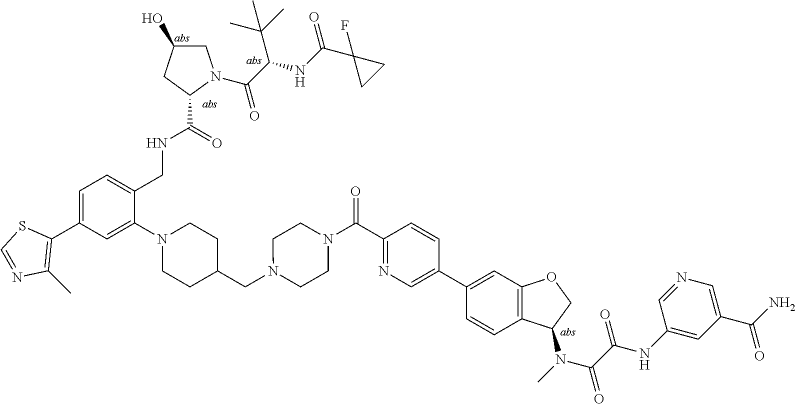 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)C(=O)Nc5cncc(C(N)=O)c5)cn4)CC3)CC2)c1] |
| A144 | 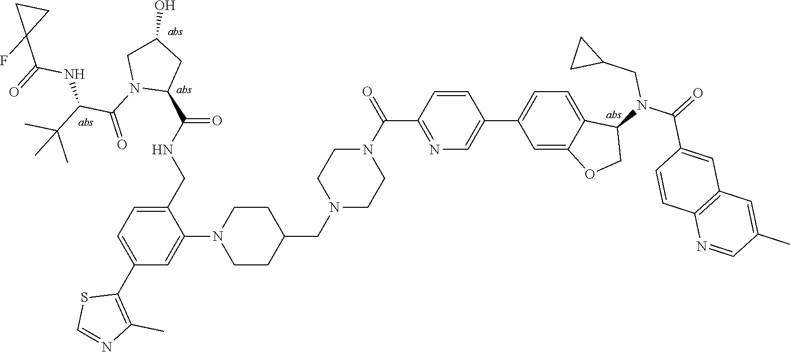 [SMILES: Cc1cnc2ccc(C(=O)N(CC3CC3)[C@H]3COc4cc(-c5ccc(C(=O)N6CCN(CC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)cc2c1] |
| A145 | 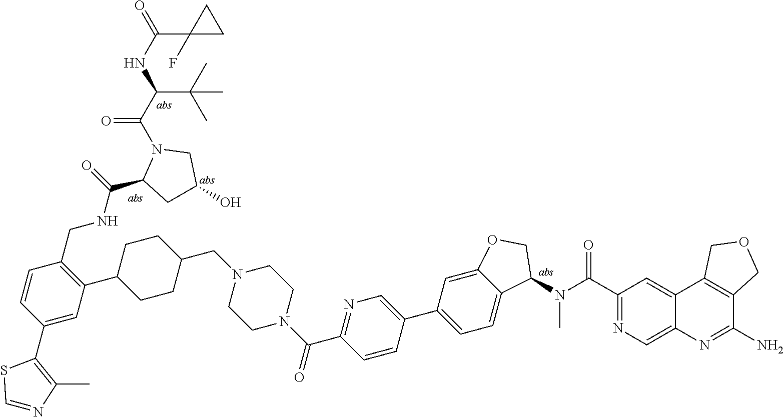 [SMILES: Cc1ncsc1-c1ccc(CNC(=O)[C@@H]2C[C@@H](O)CN2C(=O)[C@@H](NC(=O)C2(F)CC2)C(C)(C)C)c(C2CCC(CN3CCN(C(=O)c4ccc(-c5ccc6c(c5)OC[C@H]6N(C)C(=O)c5cc6c7c(c(N)nc6cn5)COC7)cn4)CC3)CC2)c1] |
| A146 | 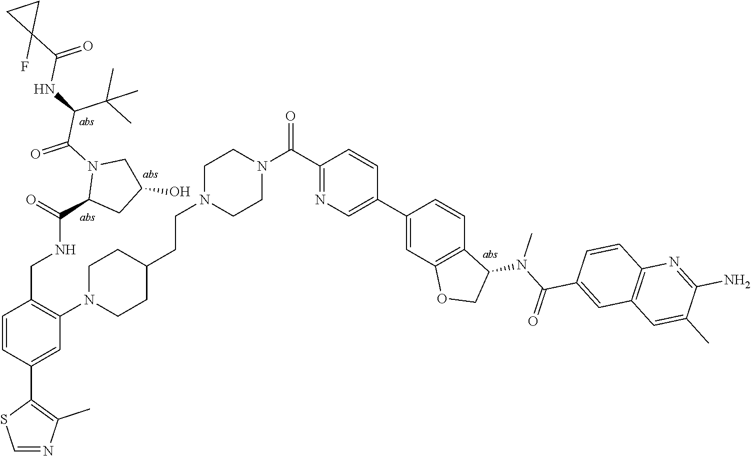 [SMILES: Cc1cc2cc(C(=O)N(C)[C@@H]3COc4cc(-c5ccc(C(=O)N6CCN(CCC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A147 | 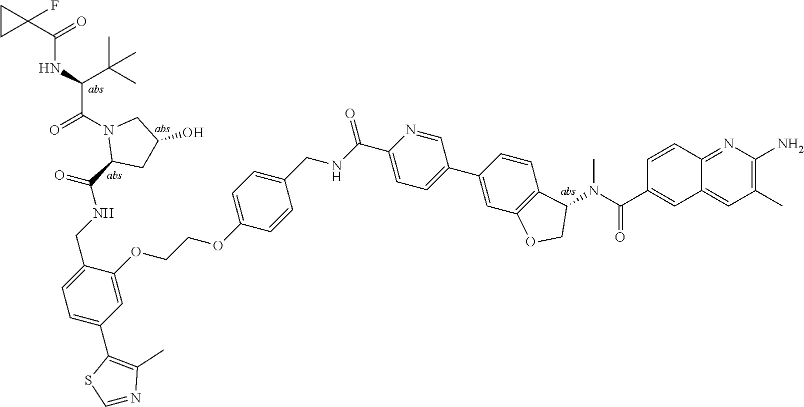 [SMILES: Cc1cc2cc(C(=O)N(C)[C@@H]3COc4cc(-c5ccc(C(=O)NCc6ccc(OCCOc7cc(-c8scnc8C)ccc7CNC(=O)[C@@H]7C[C@@H](O)CN7C(=O)[C@@H](NC(=O)C7(F)CC7)C(C)(C)C)cc6)nc5)ccc43)ccc2nc1N] |
| A148 | 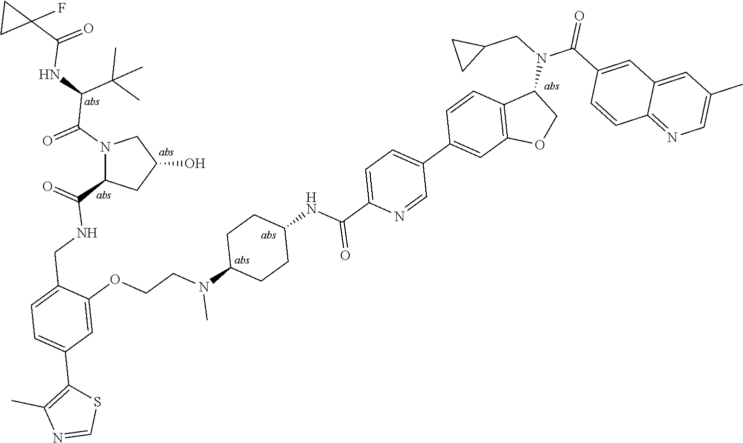 [SMILES: Cc1cnc2ccc(C(=O)N(CC3CC3)[C@@H]3COc4cc(-c5ccc(C(=O)N[C@H]6CC[C@H](N(C)CCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2c1] |
| A149 | 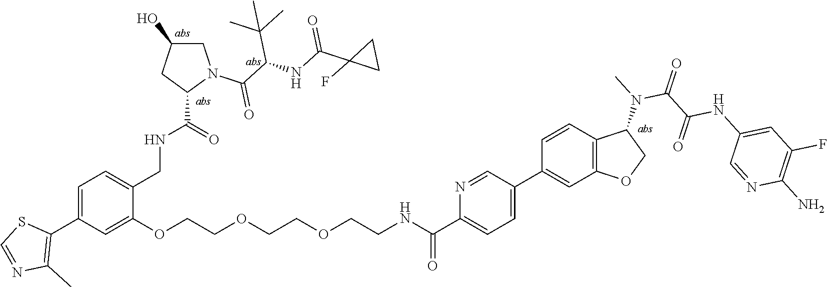 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)C(=O)Nc3cnc(N)c(F)c3)cn2)c1] |
| A150 | 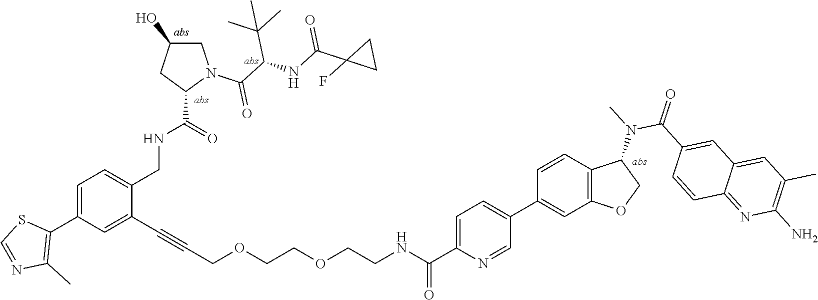 [SMILES: Cc1cc2cc(C(=O)N(C)[C@@H]3COc4cc(-c5ccc(C(=O)NCCOCCOCC#Cc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A151 | 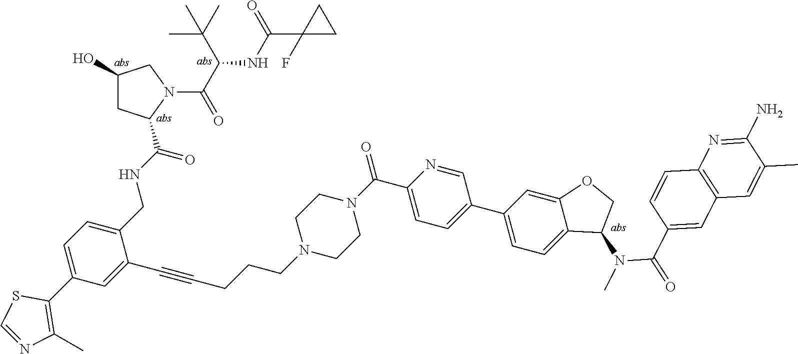 [SMILES: Cc1cc2cc(C(=O)N(C)[C@@H]3COc4cc(-c5ccc(C(=O)N6CCN(CCCC#Cc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A152 | 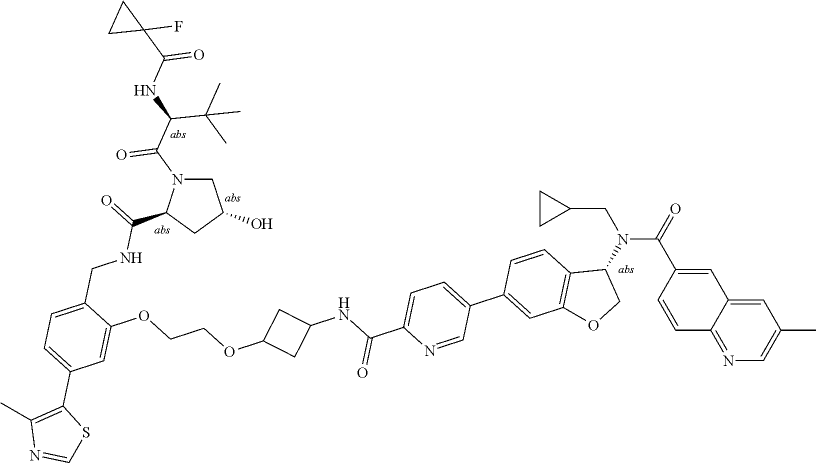 |
| A153 | 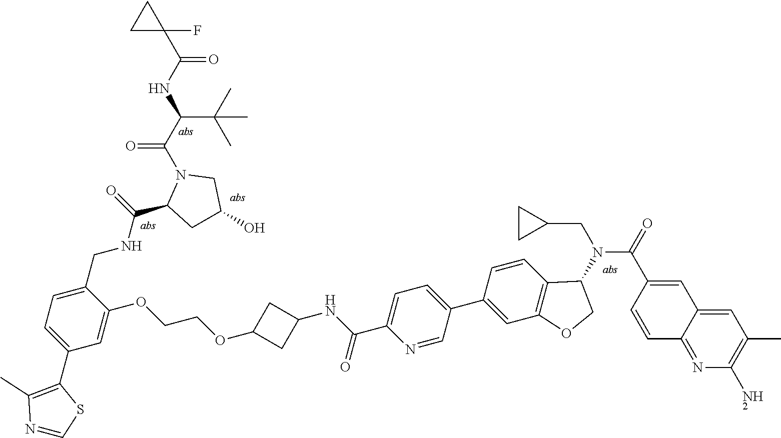 |
| A154 | 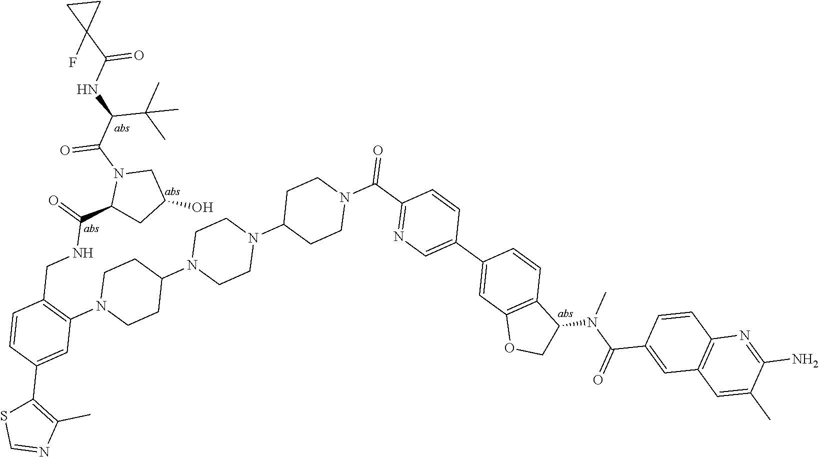 [SMILES: Cc1cc2cc(C(=O)N(C)[C@@H]3COc4cc(-c5ccc(C(=O)N6CCC(N7CCN(C8CCN(c9cc(-c%10scnc%10C)ccc9CNC(=O)[C@@H]9C[C@@H](O)CN9C(=O)[C@@H](NC(=O)C9(F)CC9)C(C)(C)C)CC8)CC7)CC6)nc5)ccc43)ccc2nc1N] |
| A155 | 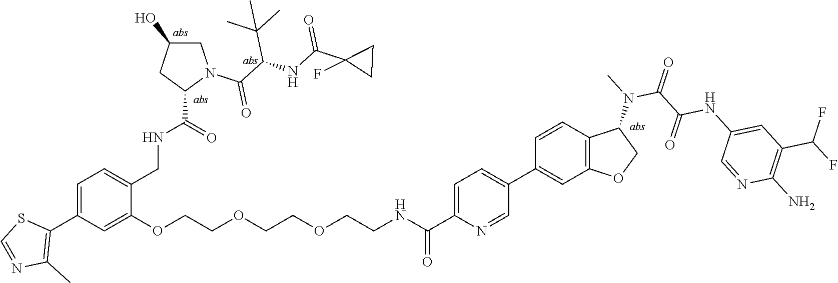 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)C(=O)Nc3cnc(N)c(C(F)F)c3)cn2)c1] |
| A156 | 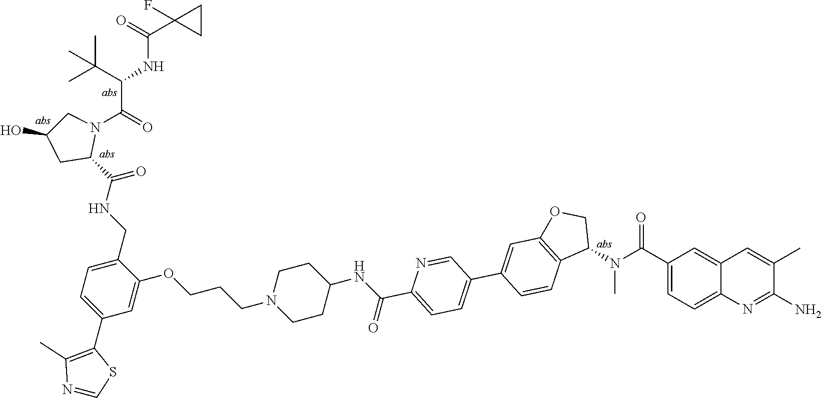 [SMILES: Cc1cc2cc(C(=O)N(C)[C@H]3COc4cc(-c5ccc(C(=O)NC6CCN(CCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A157 | 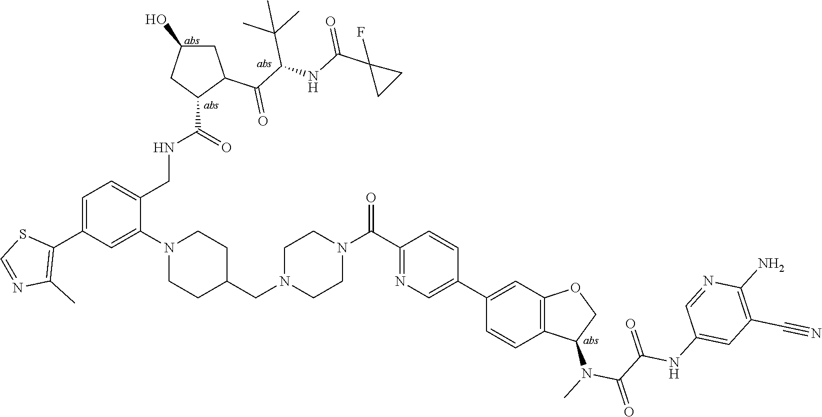 [SMILES: Cc1ncsc1-c1ccc(CNC(=O)[C@@H]2C[C@@H](O)CC2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)C(=O)Nc5cnc(N)c(C#N)c5)cn4)CC3)CC2)c1] |
| A158 | 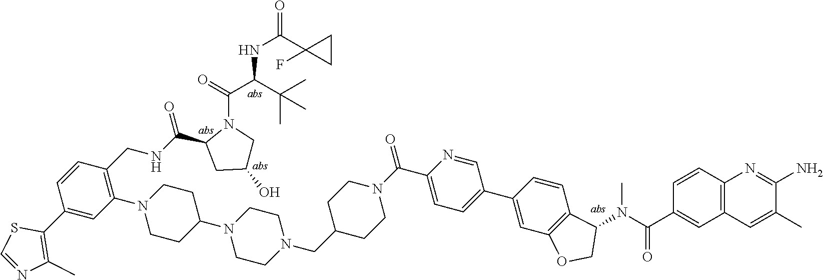 [SMILES: Cc1cc2cc(C(=O)N(C)[C@@H]3COc4cc(-c5ccc(C(=O)N6CCC(CN7CCN(C8CCN(c9cc(-c%10scnc%10C)ccc9CNC(=O)[C@@H]9C[C@@H](O)CN9C(=O)[C@@H](NC(=O)C9(F)CC9)C(C)(C)C)CC8)CC7)CC6)nc5)ccc43)ccc2nc1N] |
| A159 | 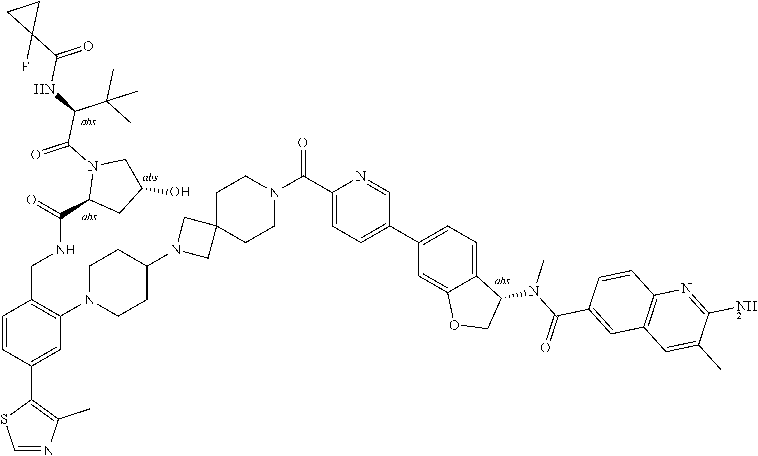 [SMILES: Cc1cc2cc(C(=O)N(C)[C@@H]3COc4cc(-c5ccc(C(=O)N6CCC7(CC6)CN(C6CCN(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A160 | 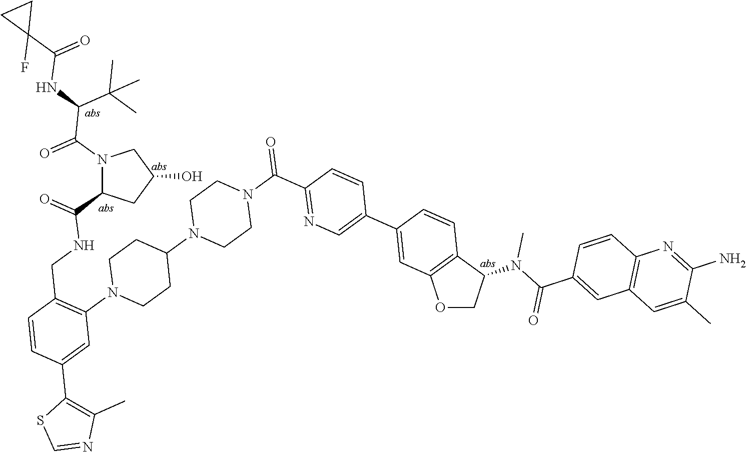 [SMILES: Cc1cc2cc(C(=O)N(C)[C@@H]3COc4cc(-c5ccc(C(=O)N6CCN(C7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A161 | 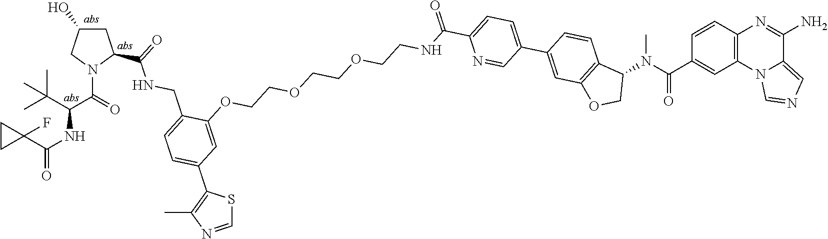 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c5cncn5c4c3)cn2)c1] |
| A162 | 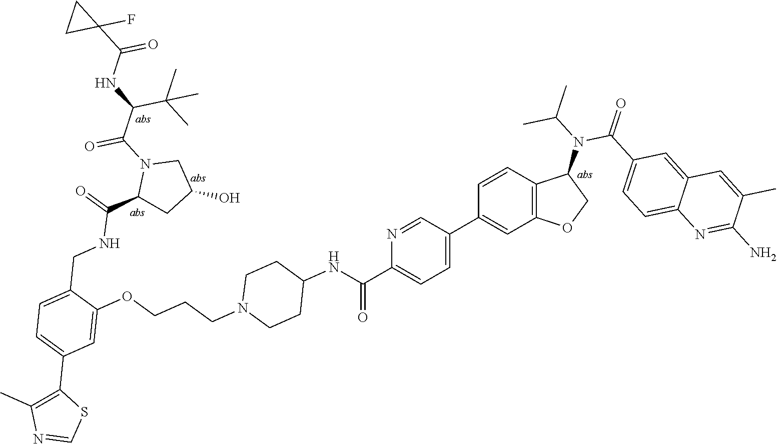 |
| A163 | 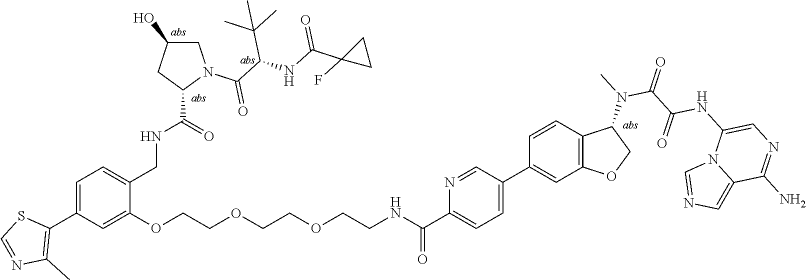 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)C(=O)Nc3cnc(N)c4cncn34)cn2)c1] |
| A164 | 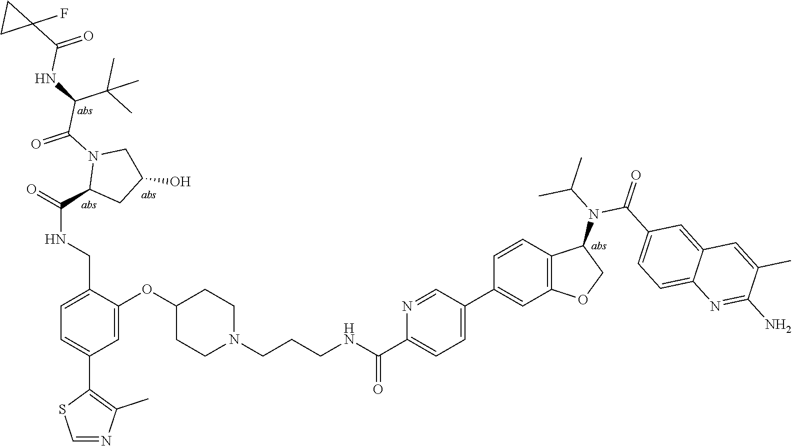 [SMILES: Cc1cc2cc(C(=O)N(C(C)C)[C@H]3COc4cc(-c5ccc(C(=O)NCCCN6CCC(Oc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A165 | 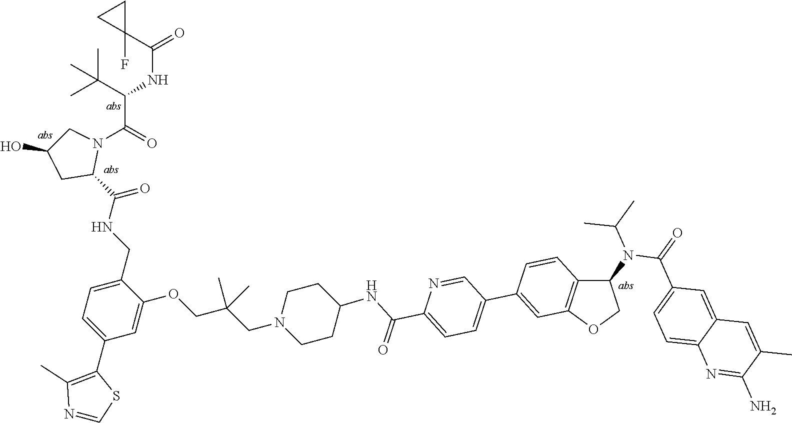 [SMILES: Cc1cc2cc(C(=O)N(C(C)C)[C@H]3COc4cc(-c5ccc(C(=O)NC6CCN(CC(C)(C)COc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A166 | 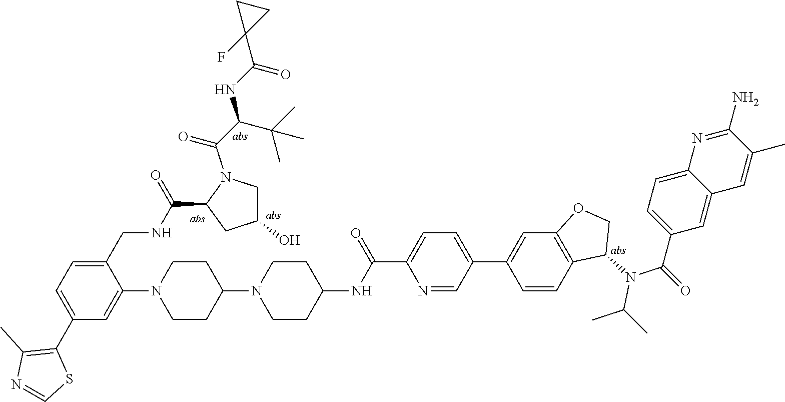 |
| A167 | 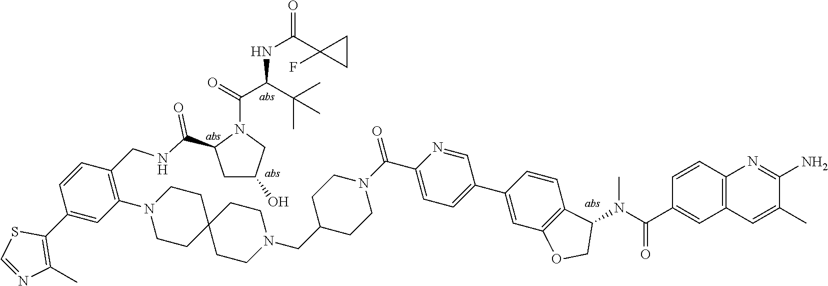 [SMILES: Cc1cc2cc(C(=O)N(C)[C@@H]3COc4cc(-c5ccc(C(=O)N6CCC(CN7CCC8(CC7)CCN(c7cc(-c9scnc9C)ccc7CNC(=O)[C@@H]7C[C@@H](O)CN7C(=O)[C@@H](NC(=O)C7(F)CC7)C(C)(C)C)CC8)CC6)nc5)ccc43)ccc2nc1N] |
| A168 | 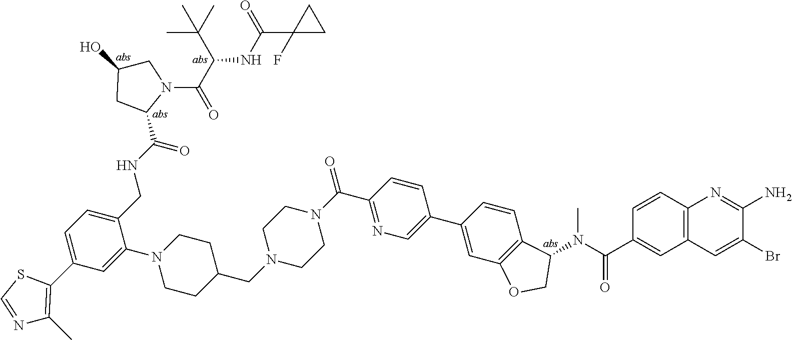 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(Br)cc6c5)cn4)CC3)CC2)c1] |
| A169 | 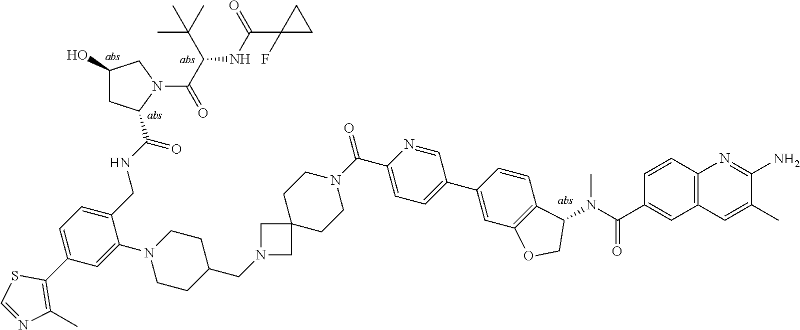 [SMILES: Cc1cc2cc(C(=O)N(C)[C@@H]3COc4cc(-c5ccc(C(=O)N6CCC7(CC6)CN(CC6CCN(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A170 | 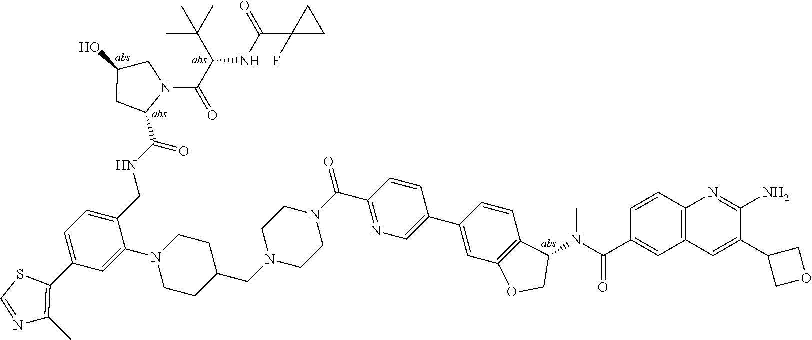 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C7COC7)cc6c5)cn4)CC3)CC2)c1] |
| A171 | 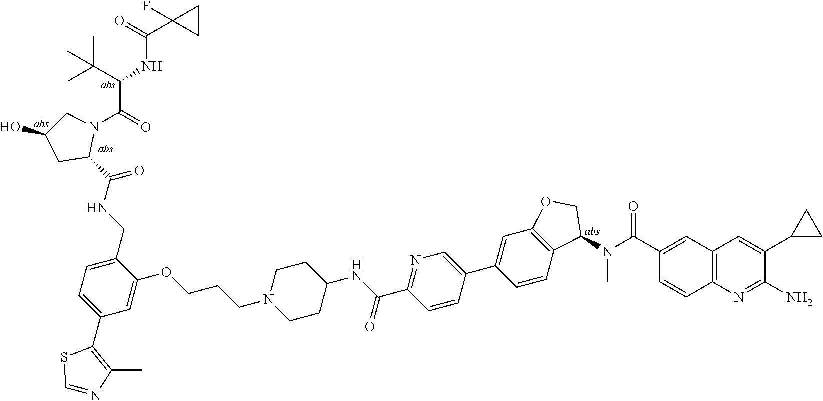 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCN2CCC(NC(=O)c3ccc(-c4ccc5c(c4)OC[C@H]5N(C)C(=O)c4ccc5nc(N)c(C6CC6)cc5c4)cn3)CC2)c1] |
| A172 | 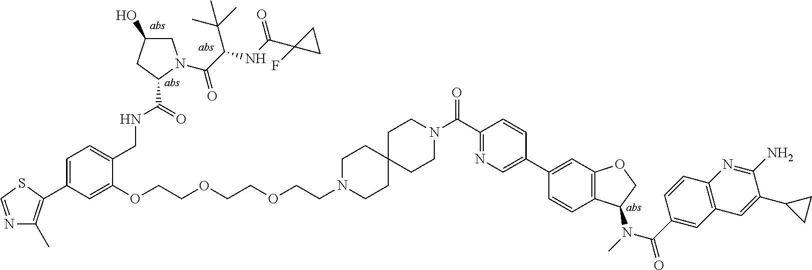 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCN2CCC3(CC2)CCN(C(=O)c2ccc(-c4ccc5c(c4)OC[C@H]5N(C)C(=O)c4ccc5nc(N)c(C6CC6)cc5c4)cn2)CC3)c1] |
| A173 | 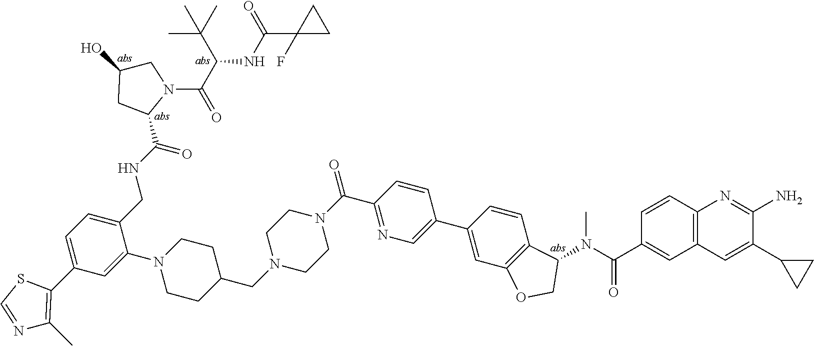 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C7CC7)cc6c5)cn4)CC3)CC2)c1] |
| A174 | 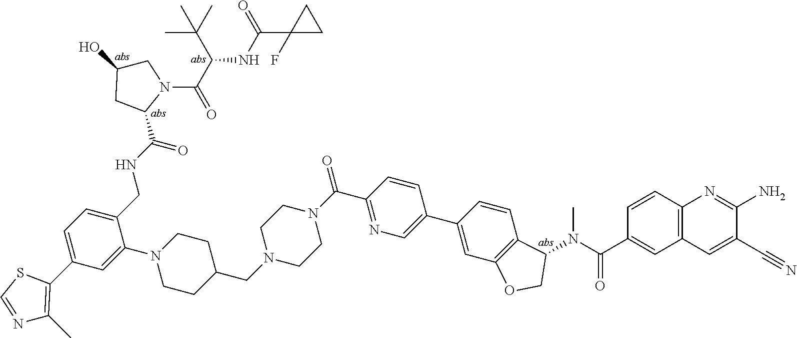 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C#N)cc6c5)cn4)CC3)CC2)c1] |
| A175 | 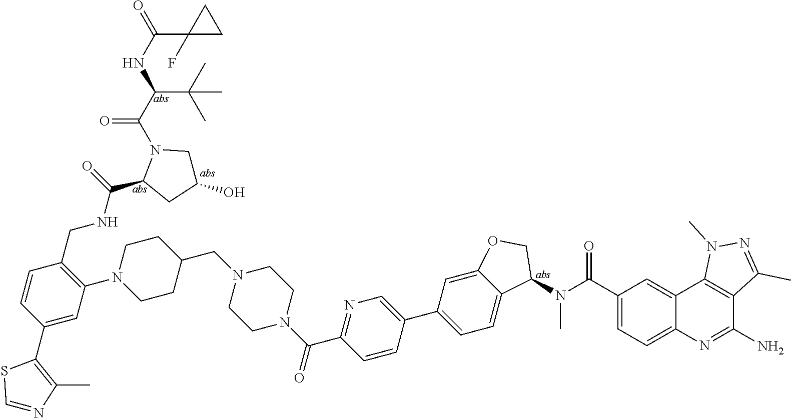 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7c(C)nn(C)c7c6c5)cn4)CC3)CC2)c1] |
| A176 | 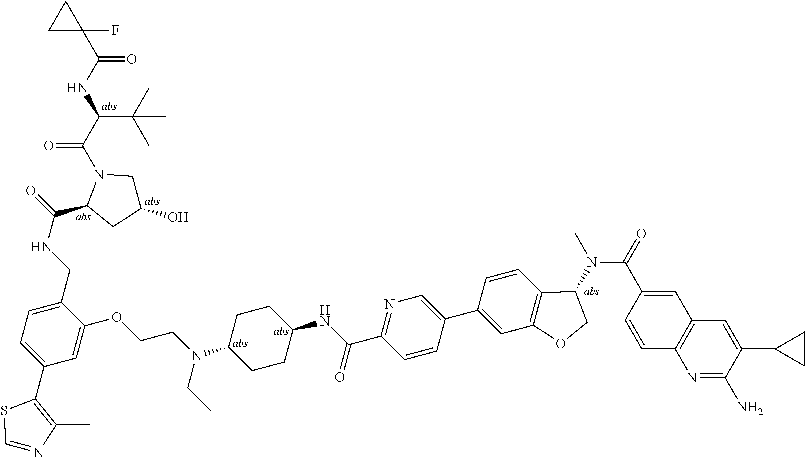 [SMILES: CCN(CCOc1cc(-c2scnc2C)ccc1CNC(=O)[C@@H]1C[C@@H](O)CN1C(=O)[C@@H](NC(=O)C1(F)CC1)C(C)(C)C)[C@H]1CC[C@H](NC(=O)c2ccc(-c3ccc4c(c3)OC[C@H]4N(C)C(=O)c3ccc4nc(N)c(C5CC5)cc4c3)cn2)CC1] |
| A177 | 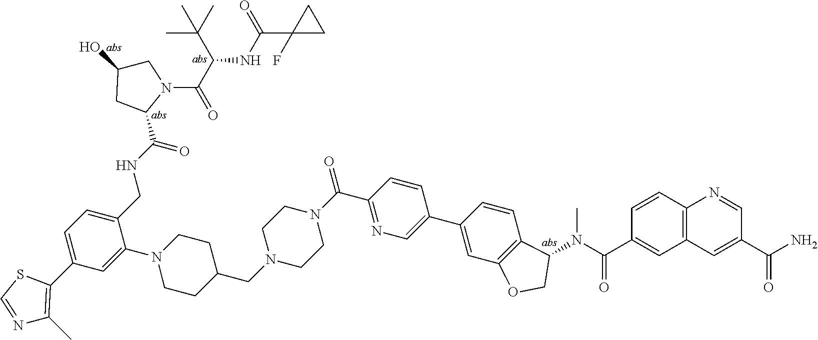 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncc(C(N)=O)cc6c5)cn4)CC3)CC2)c1] |
| A178 | 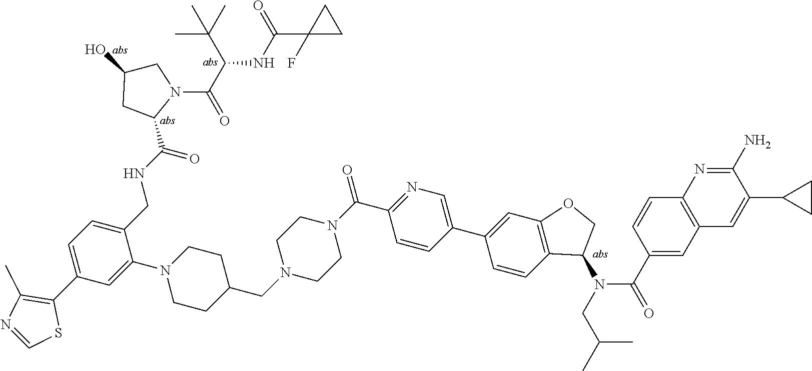 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(CC(C)C)C(=O)c5ccc6nc(N)c(C7CC7)cc6c5)cn4)CC3)CC2)c1] |
| A179 | 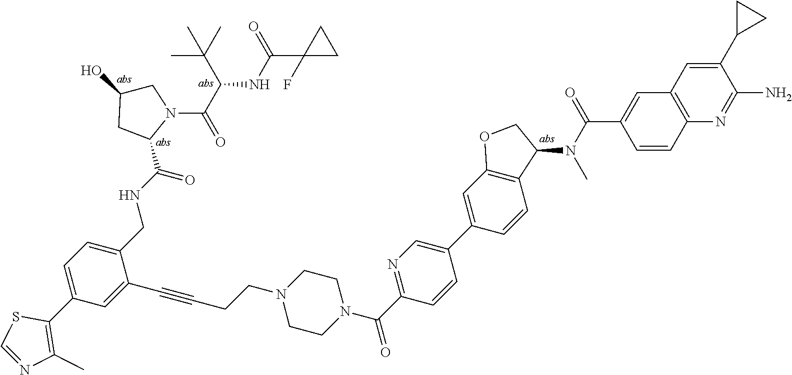 [SMILES: Cc1ncsc1-c1ccc(CNC(=O)[C@@H]2C[C@@H](O)CN2C(=O)[C@@H](NC(=O)C2(F)CC2)C(C)(C)C)c(C#CCCN2CCN(C(=O)c3ccc(-c4ccc5c(c4)OC[C@H]5N(C)C(=O)c4ccc5nc(N)c(C6CC6)cc5c4)cn3)CC2)c1] |
| A180 | 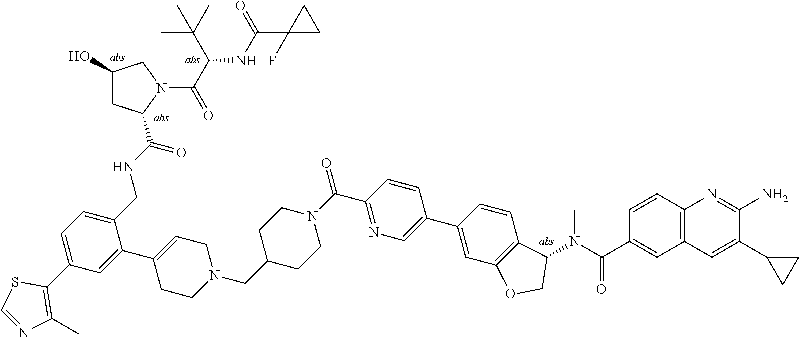 [SMILES: Cc1ncsc1-c1ccc(CNC(=O)[C@@H]2C[C@@H](O)CN2C(=O)[C@@H](NC(=O)C2(F)CC2)C(C)(C)C)c(C2=CCN(CC3CCN(C(=O)c4ccc(-c5ccc6c(c5)OC[C@H]6N(C)C(=O)c5ccc6nc(N)c(C7CC7)cc6c5)cn4)CC3)CC2)c1] |
| A181 | 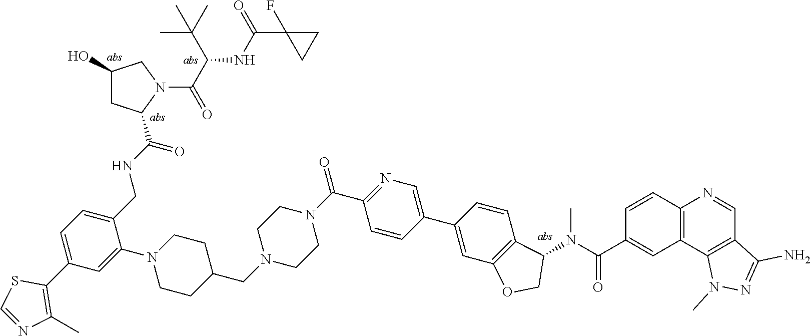 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncc7c(N)nn(C)c7c6c5)cn4)CC3)CC2)c1] |
| A182 | 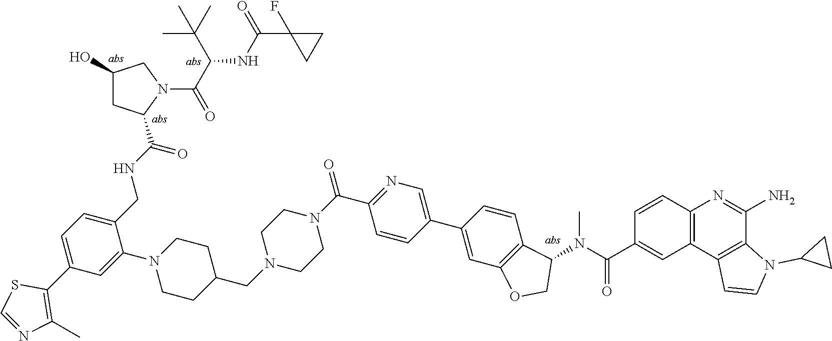 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7c(ccn7C7CC7)c6c5)cn4)CC3)CC2)c1] |
| A183 | 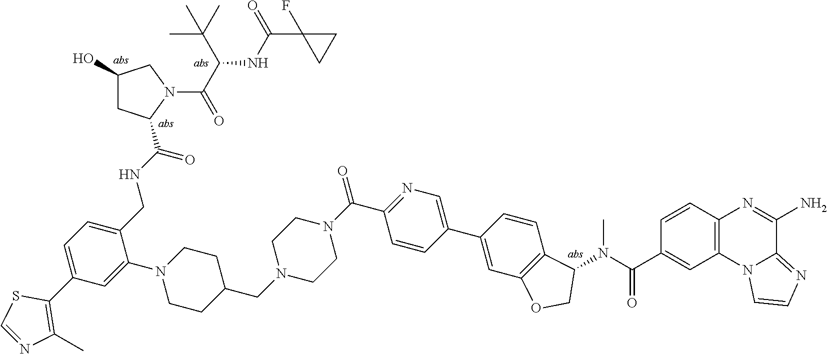 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7nccn7c6c5)cn4)CC3)CC2)c1] |
| A184 | 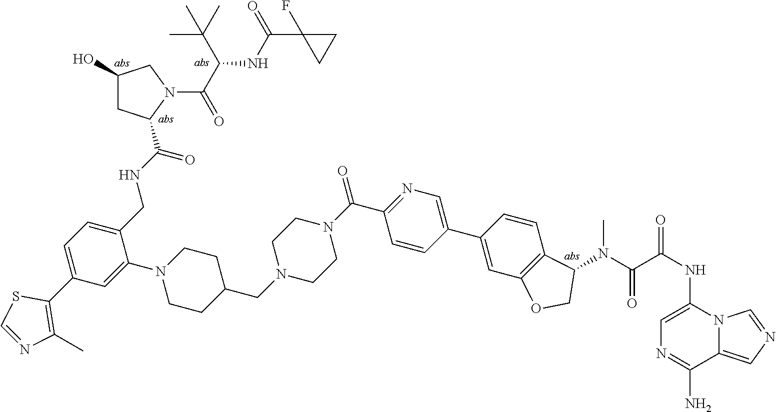 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)C(=O)Nc5cnc(N)c6cncn56)cn4)CC3)CC2)c1] |
| A185 | 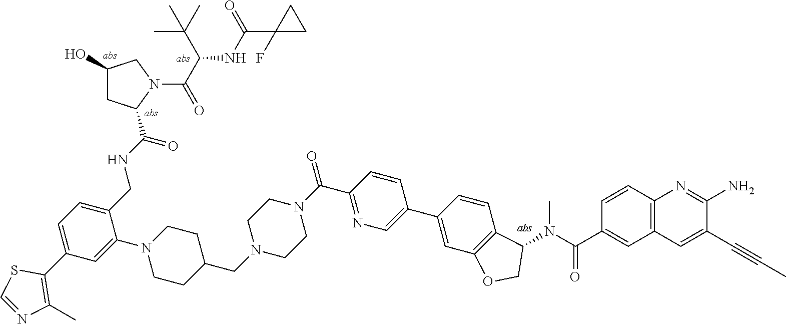 [SMILES: CC#Cc1cc2cc(C(=O)N(C)[C@@H]3COc4cc(-c5ccc(C(=O)N6CCN(CC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A186 | 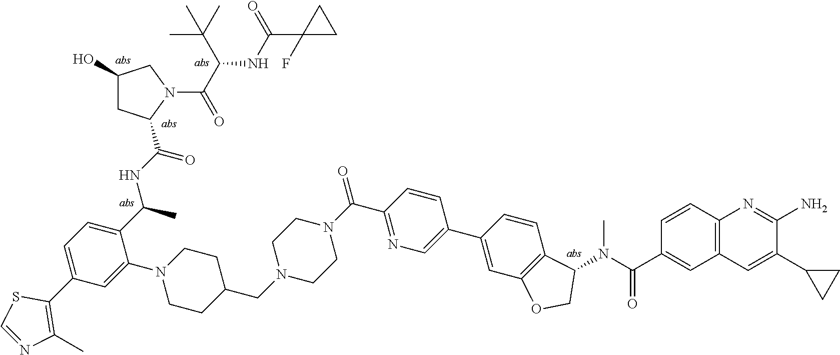 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C7CC7)cc6c5)cn4)CC3)CC2)c1] |
| A187 | 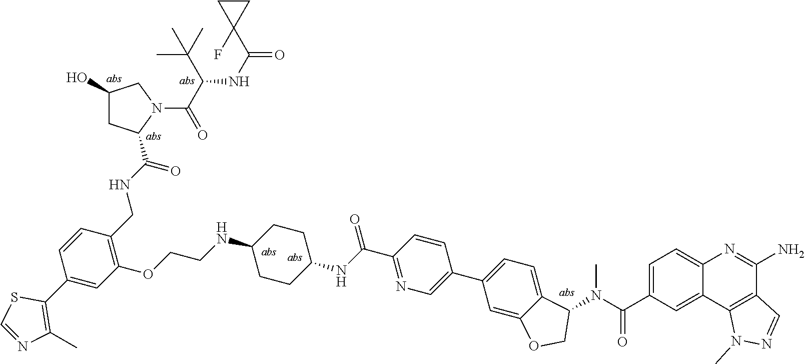 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN[C@H]2CC[C@H](NC(=O)c3ccc(-c4ccc5c(c4)OC[C@H]5N(C)C(=O)c4ccc5nc(N)c6cnn(C)c6c5c4)cn3)CC2)c1] |
| A188 | 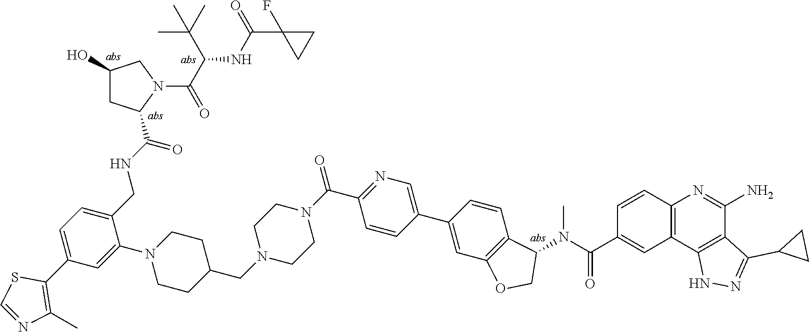 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7c(C8CC8)n[nH]c7c6c5)cn4)CC3)CC2)c1] |
| A189 | 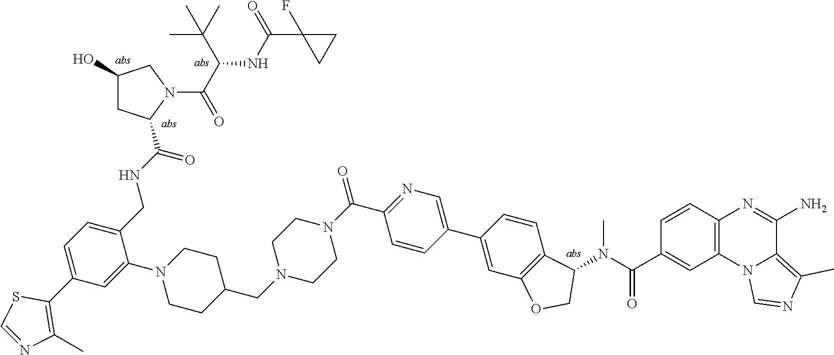 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7c(C)ncn7c6c5)cn4)CC3)CC2)c1] |
| A190 | 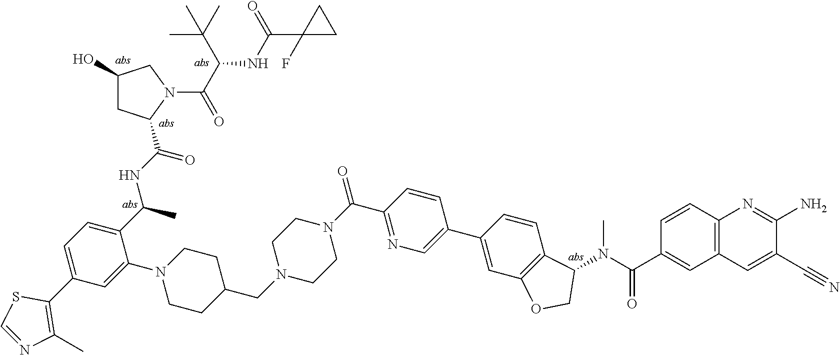 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C#N)cc6c5)cn4)CC3)CC2)c1] |
| A192 | 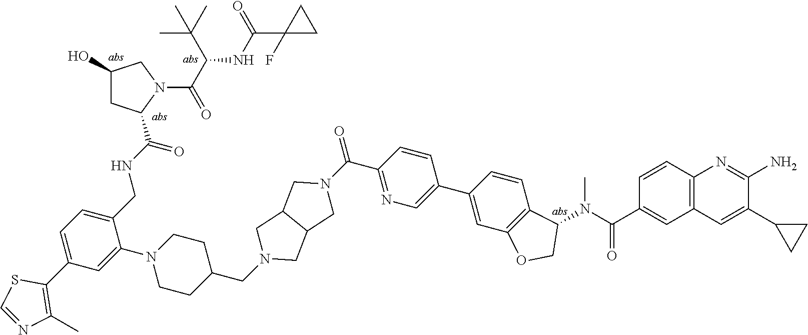 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4CN(C(=O)c5ccc(-c6ccc7c(c6)OC[C@H]7N(C)C(=O)c6ccc7nc(N)c(C8CC8)cc7c6)cn5)CC4C3)CC2)c1] |
| A193 | 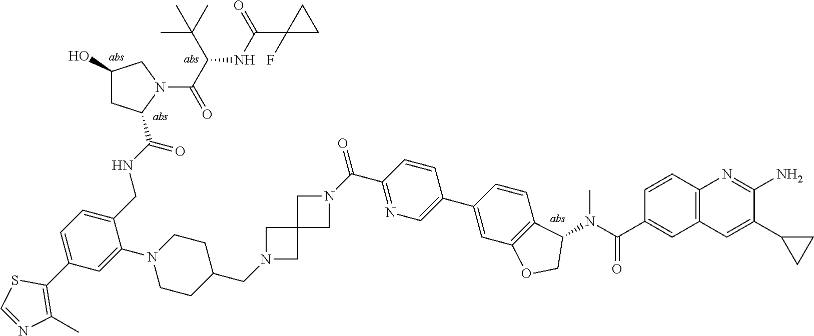 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4(C3)CN(C(=O)c3ccc(-c5ccc6c(c5)OC[C@H]6N(C)C(=O)c5ccc6nc(N)c(C7CC7)cc6c5)cn3)C4)CC2)c1] |
| A194 | 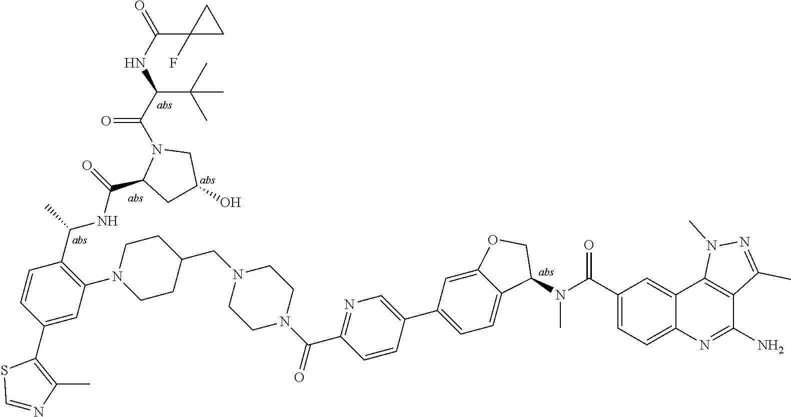 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7c(C)nn(C)c7c6c5)cn4)CC3)CC2)c1] |
| A196 | 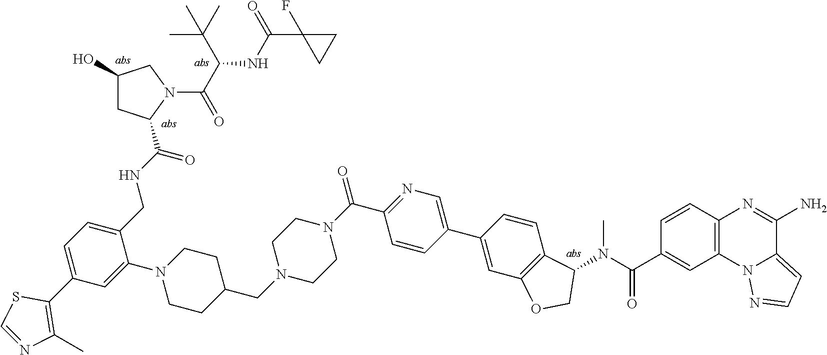 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7ccnn7c6c5)cn4)CC3)CC2)c1] |
| A198 | 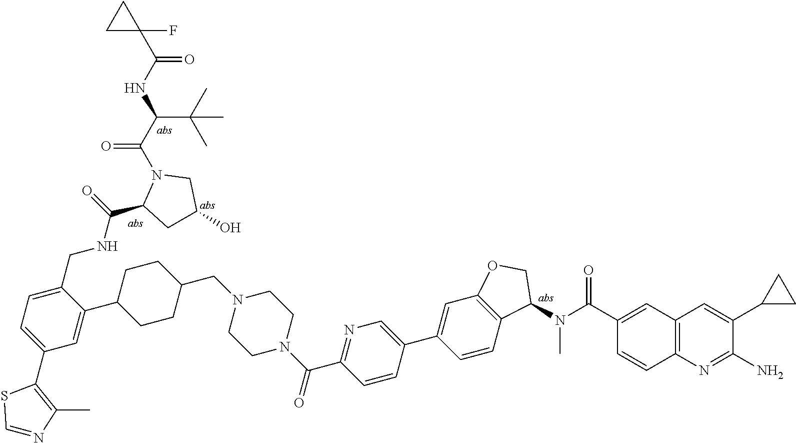 [SMILES: Cc1ncsc1-c1ccc(CNC(=O)[C@@H]2C[C@@H](O)CN2C(=O)[C@@H](NC(=O)C2(F)CC2)C(C)(C)C)c(C2CCC(CN3CCN(C(=O)c4ccc(-c5ccc6c(c5)OC[C@H]6N(C)C(=O)c5ccc6nc(N)c(C7CC7)cc6c5)cn4)CC3)CC2)c1] |
| A199 | 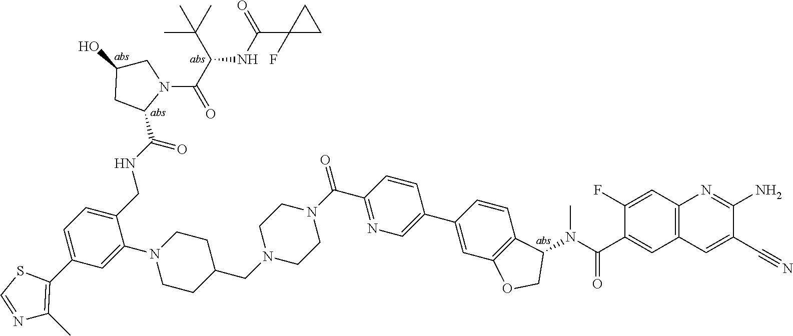 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5cc6cc(C#N)c(N)nc6cc5F)cn4)CC3)CC2)c1] |
| A200 | 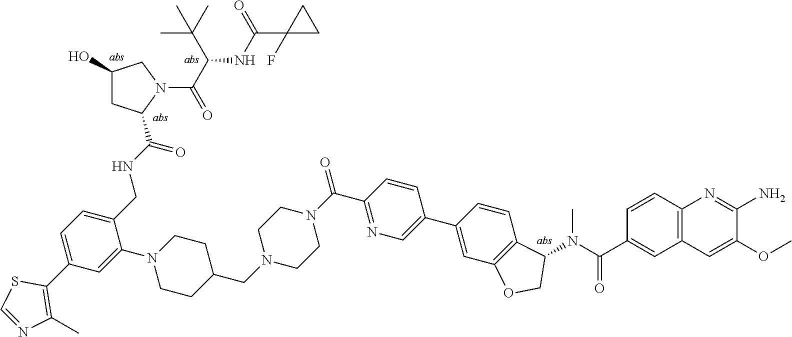 [SMILES: COc1cc2cc(C(=O)N(C)[C@@H]3COc4cc(-c5ccc(C(=O)N6CCN(CC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A201 | 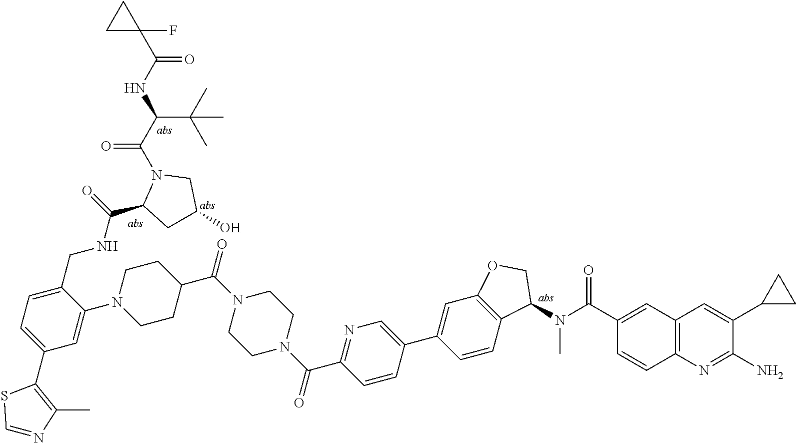 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(C(=O)N3CCN(C(=O)c4ccc(-c5ccc6c(c5)OC[C@H]6N(C)C(=O)c5ccc6nc(N)c(C7CC7)cc6c5)cn4)CC3)CC2)c1] |
| A202 | 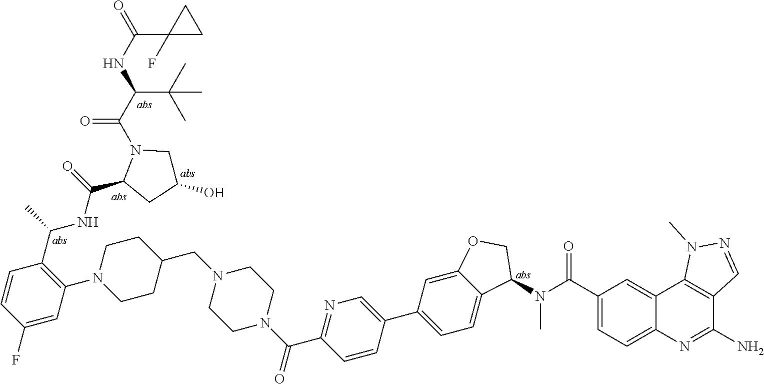 [SMILES: C[C@H](NC(=O)[C@@H]1C[C@@H](O)CN1C(=O)[C@@H](NC(=O)C1(F)CC1)C(C)(C)C)c1ccc(F)cc1N1CCC(CN2CCN(C(=O)c3ccc(-c4ccc5c(c4)OC[C@H]5N(C)C(=O)c4ccc5nc(N)c6cnn(C)c6c5c4)cn3)CC2)CC1] |
| A203 | 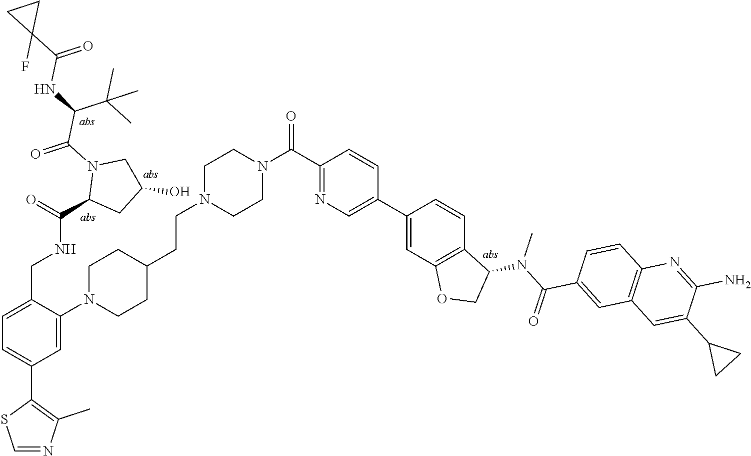 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CCN3CCN(C(=O)c4ccc(-c5ccc6c(c5)OC[C@H]6N(C)C(=O)c5ccc6nc(N)c(C7CC7)cc6c5)cn4)CC3)CC2)c1] |
| A204 | 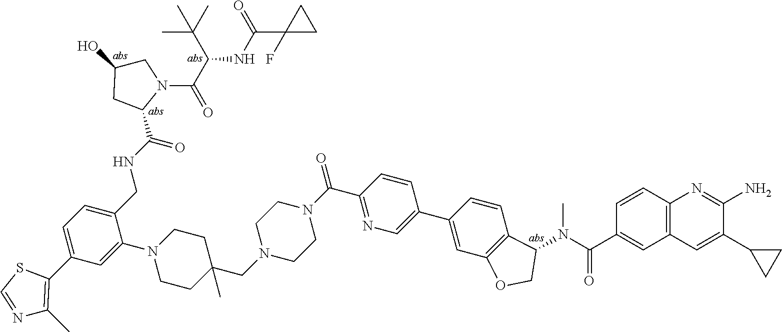 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(C)(CN3CCN(C(=O)c4ccc(-c5ccc6c(c5)OC[C@H]6N(C)C(=O)c5ccc6nc(N)c(C7CC7)cc6c5)cn4)CC3)CC2)c1] |
| A206 | 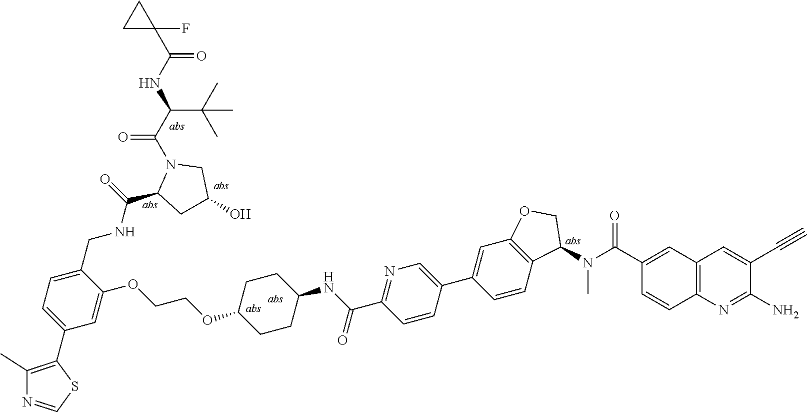 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A207 | 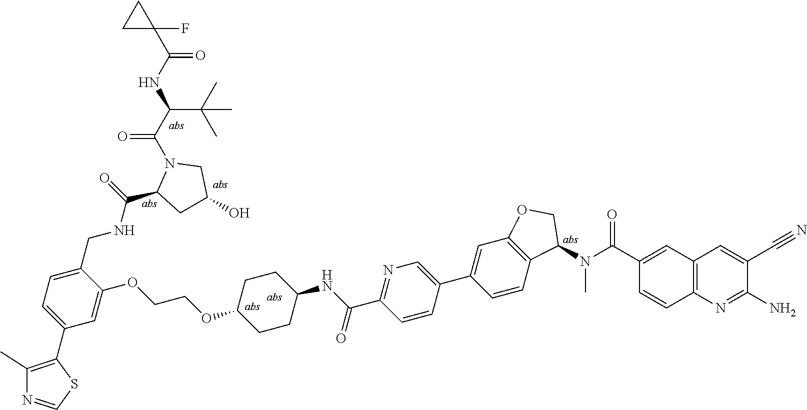 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A208 | 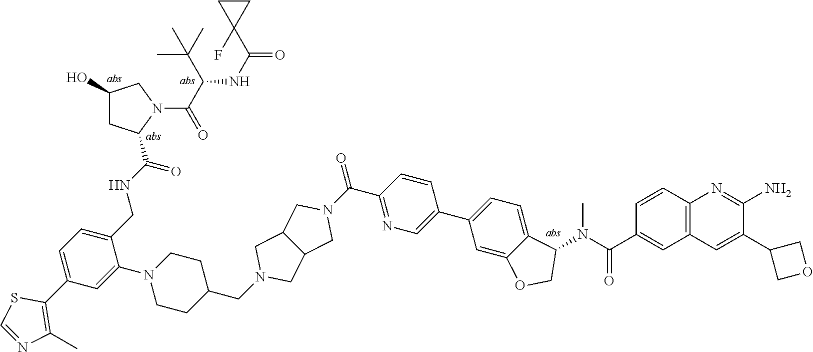 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4CN(C(=O)c5ccc(-c6ccc7c(c6)OC[C@H]7N(C)C(=O)c6ccc7nc(N)c(C8COC8)cc7c6)cn5)CC4C3)CC2)c1] |
| A209 | 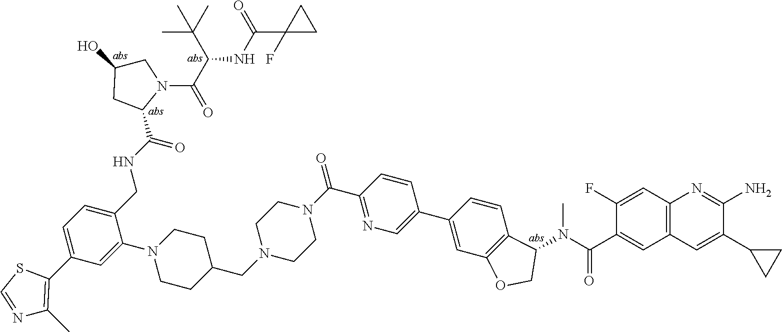 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5cc6cc(C7CC7)c(N)nc6cc5F)cn4)CC3)CC2)c1] |
| A210 | 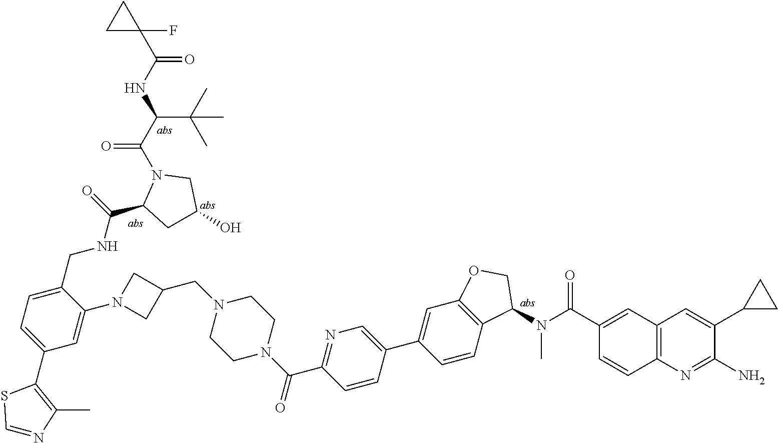 [SMILES: Cc1ncsc1-c1ccc(CNC(=O)[C@@H]2C[C@@H](O)CN2C(=O)[C@@H](NC(=O)C2(F)CC2)C(C)(C)C)c(N2CC(CN3CCN(C(=O)c4ccc(-c5ccc6c(c5)OC[C@H]6N(C)C(=O)c5ccc6nc(N)c(C7CC7)cc6c5)cn4)CC3)C2)c1] |
| A212 | 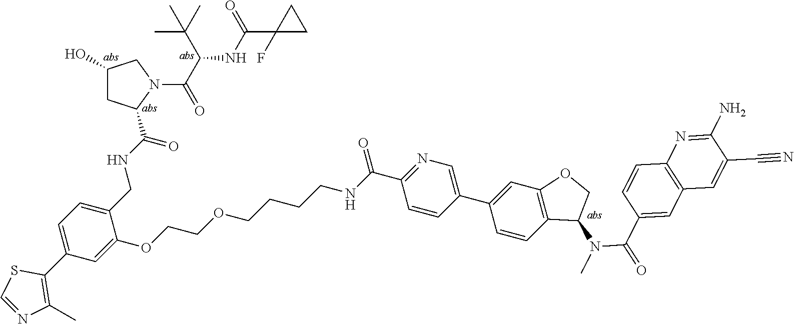 [SMILES: Cc1ncsc1-c1ccc(CNC(=O)[C@@H]2C[C@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(C#N)cc4c3)cn2)c1] |
| A213 | 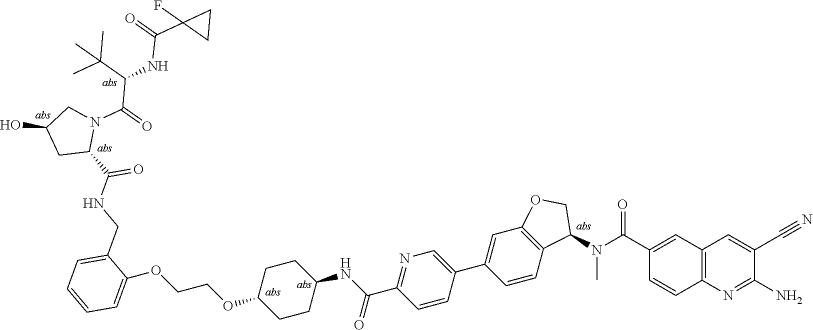 [SMILES: CN(C(=O)c1ccc2nc(N)c(C#N)cc2c1)[C@@H]1COc2cc(-c3ccc(C(=O)N[C@H]4CC[C@H](OCCOc5ccccc5CNC(=O)[C@@H]5C[C@@H](O)CN5C(=O)[C@@H](NC(=O)C5(F)CC5)C(C)(C)C)CC4)nc3)ccc21] |
| A214 | 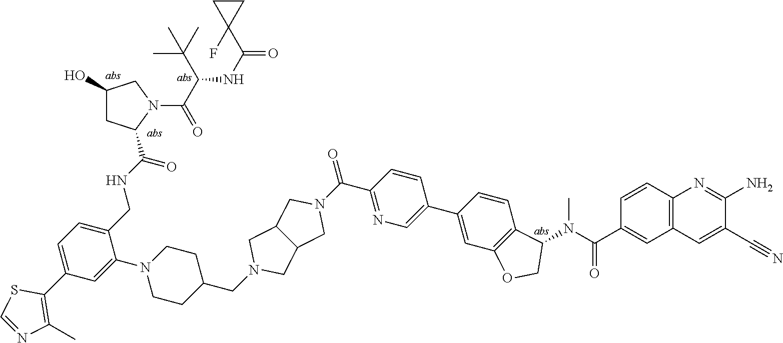 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4CN(C(=O)c5ccc(-c6ccc7c(c6)OC[C@H]7N(C)C(=O)c6ccc7nc(N)c(C#N)cc7c6)cn5)CC4C3)CC2)c1] |
| A215 | 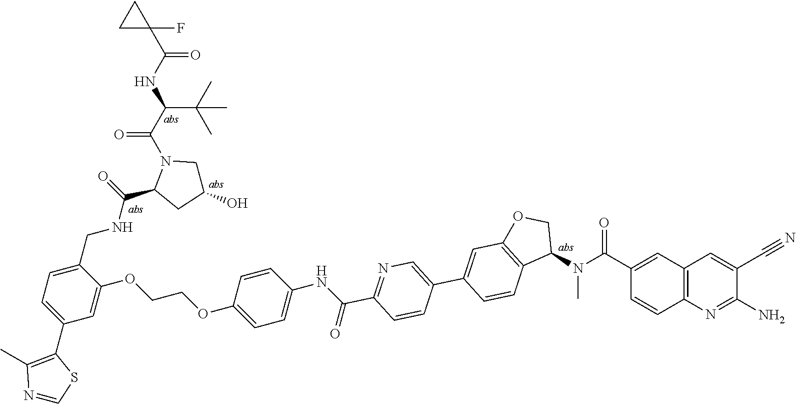 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOc2ccc(NC(=O)c3ccc(-c4ccc5c(c4)OC[C@H]5N(C)C(=O)c4ccc5nc(N)c(C#N)cc5c4)cn3)cc2)c1] |
| A216 | 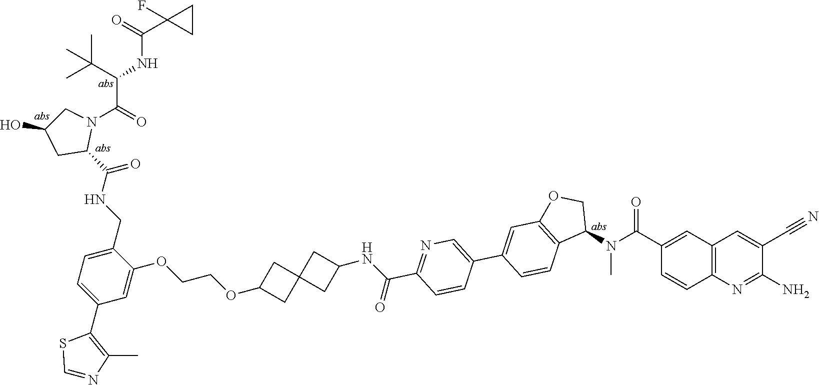 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOC2CC3(CC(NC(=O)c4ccc(-c5ccc6c(c5)OC[C@H]6N(C)C(=O)c5ccc6nc(N)c(C#N)cc6c5)cn4)C3)C2)c1] |
| A217 | 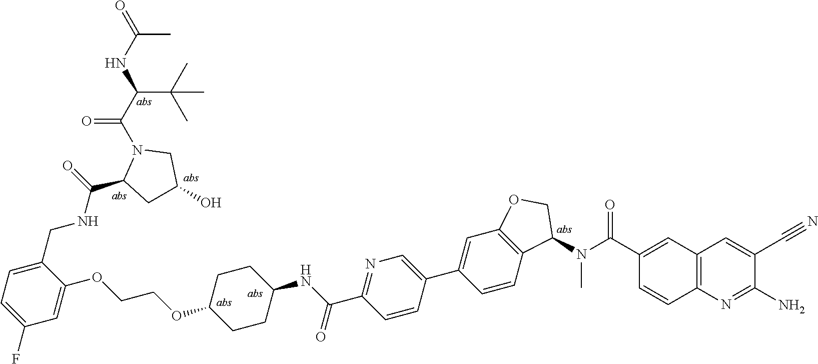 [SMILES: CC(=O)N[C@H](C(=O)N1C[C@H](O)C[C@H]1C(=O)NCc1ccc(F)cc1OCCO[C@H]1CC[C@H](NC(=O)c2ccc(-c3ccc4c(c3)OC[C@H]4N(C)C(=O)c3ccc4nc(N)c(C#N)cc4c3)cn2)CC1)C(C)(C)C] |
| A218 | 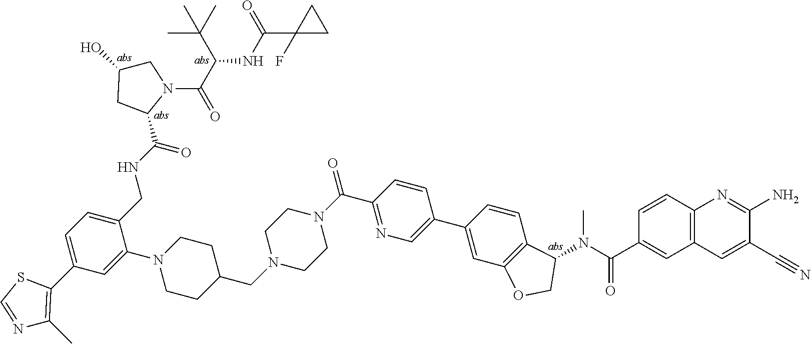 [SMILES: Cc1ncsc1-c1ccc(CNC(=O)[C@@H]2C[C@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C#N)cc6c5)cn4)CC3)CC2)c1] |
| A219 | 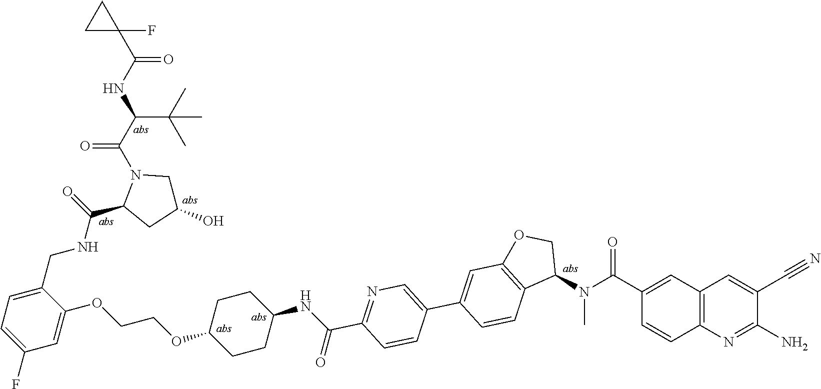 [SMILES: CN(C(=O)c1ccc2nc(N)c(C#N)cc2c1)[C@@H]1COc2cc(-c3ccc(C(=O)N[C@H]4CC[C@H](OCCOc5cc(F)ccc5CNC(=O)[C@@H]5C[C@@H](O)CN5C(=O)[C@@H](NC(=O)C5(F)CC5)C(C)(C)C)CC4)nc3)ccc21] |
| A220 | 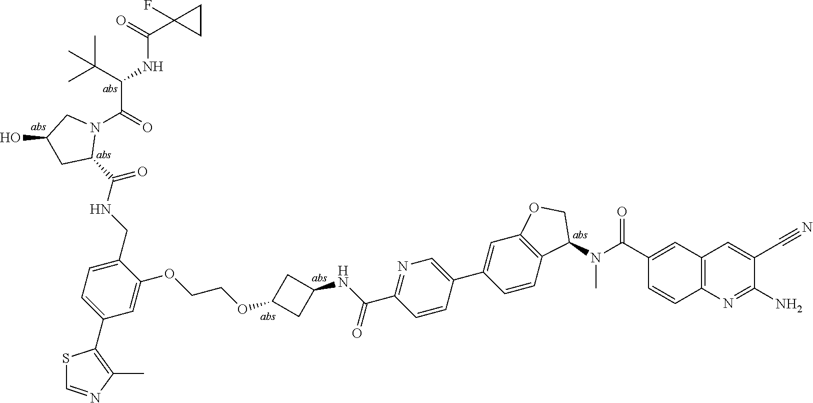 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2C[C@H](NC(=O)c3ccc(-c4ccc5c(c4)OC[C@H]5N(C)C(=O)c4ccc5nc(N)c(C#N)cc5c4)cn3)C2)c1] |
| A221 | 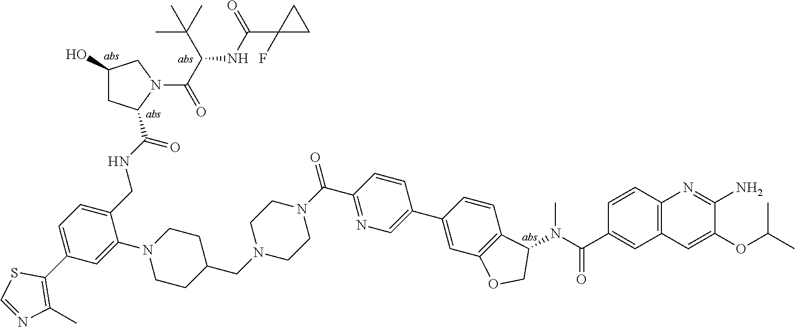 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(OC(C)C)cc6c5)cn4)CC3)CC2)c1] |
| A222 | 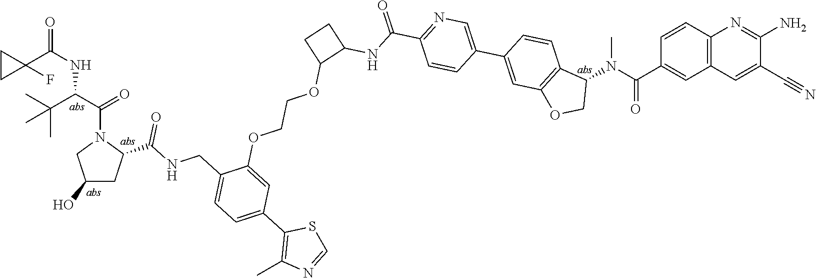 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOC2CCC2NC(=O)c2ccc(-c3ccc4c(c3)OC[C@H]4N(C)C(=O)c3ccc4nc(N)c(C#N)cc4c3)cn2)c1] |
| A223 | 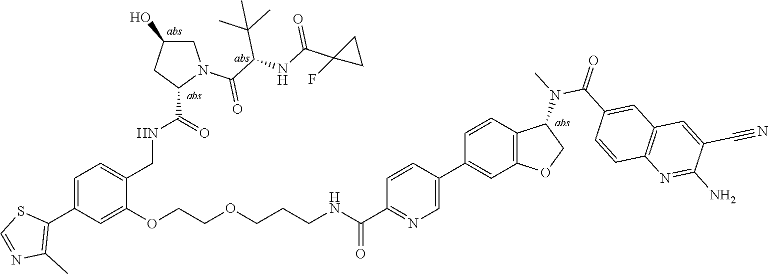 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCNC(=O)c2ccc(-c3ccc4c(c3)OC[C@H]4N(C)C(=O)c3ccc4nc(N)c(C#N)cc4c3)cn2)c1] |
| A224 | 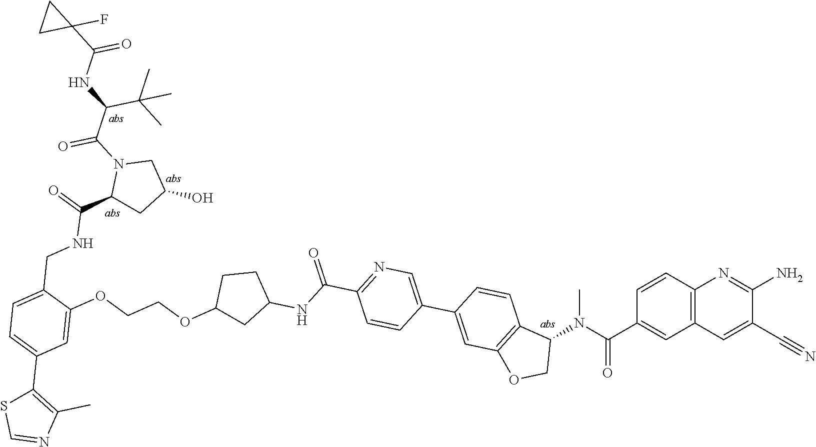 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOC2CCC(NC(=O)c3ccc(-c4ccc5c(c4)OC[C@H]5N(C)C(=O)c4ccc5nc(N)c(C#N)cc5c4)cn3)C2)c1] |
| A225 | 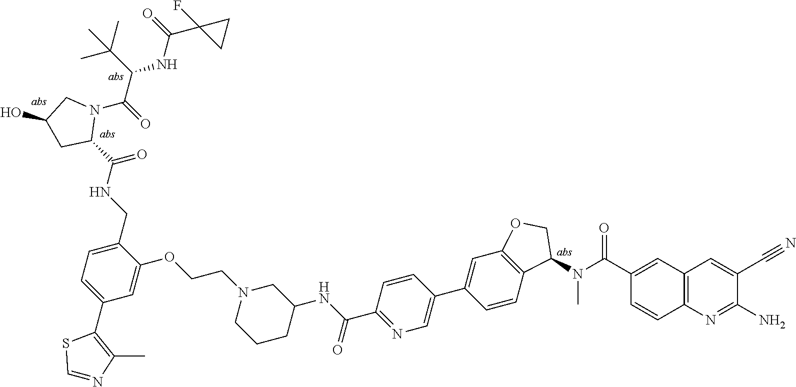 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCCC(NC(=O)c3ccc(-c4ccc5c(c4)OC[C@H]5N(C)C(=O)c4ccc5nc(N)c(C#N)cc5c4)cn3)C2)c1] |
| A226 | 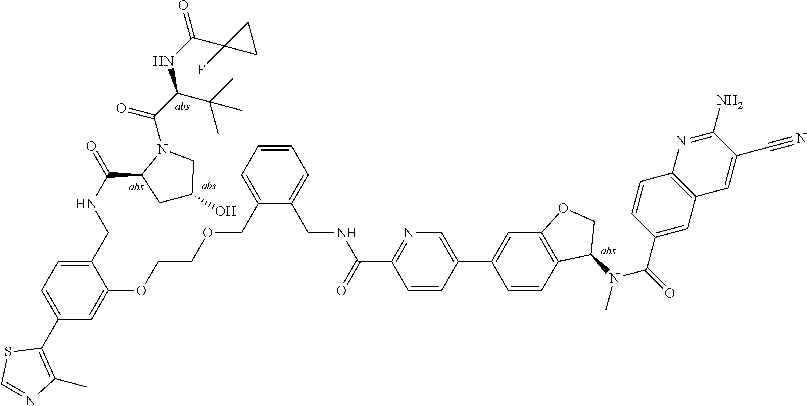 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCc2ccccc2CNC(=O)c2ccc(-c3ccc4c(c3)OC[C@H]4N(C)C(=O)c3ccc4nc(N)c(C#N)cc4c3)cn2)c1] |
| A227 | 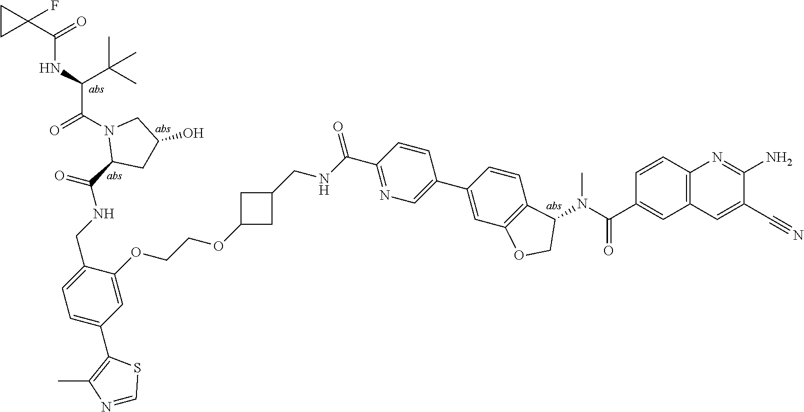 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOC2CC(CNC(=O)c3ccc(-c4ccc5c(c4)OC[C@H]5N(C)C(=O)c4ccc5nc(N)c(C#N)cc5c4)cn3)C2)c1] |
| A228 | 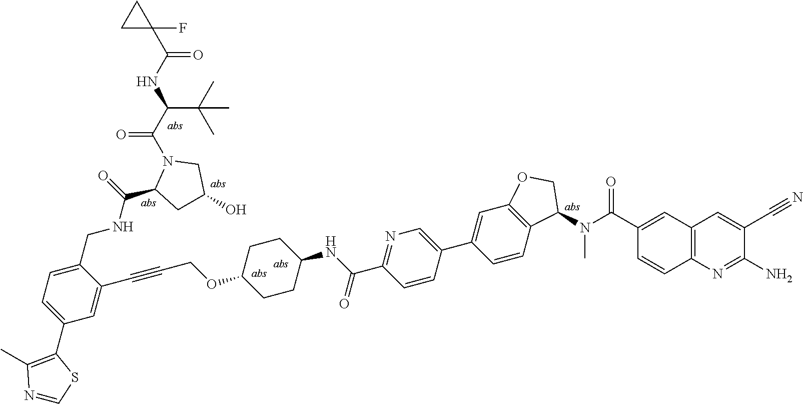 [SMILES: Cc1ncsc1-c1ccc(CNC(=O)[C@@H]2C[C@@H](O)CN2C(=O)[C@@H](NC(=O)C2(F)CC2)C(C)(C)C)c(C#CCO[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A229 | 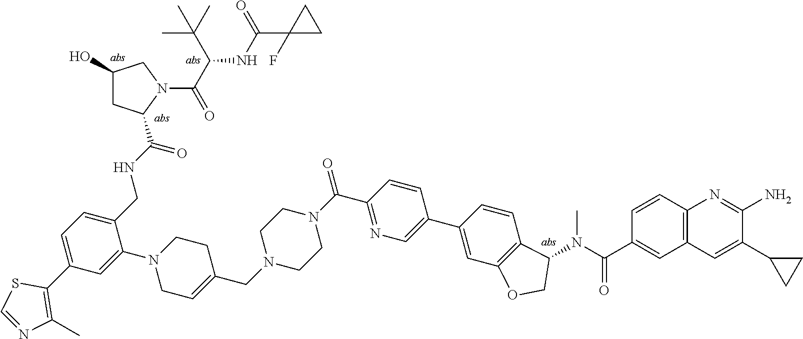 [SMILES: Cc1ncsc1-c1ccc(CNC(=O)[C@@H]2C[C@@H](O)CN2C(=O)[C@@H](NC(=O)C2(F)CC2)C(C)(C)C)c(N2CC=C(CN3CCN(C(=O)c4ccc(-c5ccc6c(c5)OC[C@H]6N(C)C(=O)c5ccc6nc(N)c(C7CC7)cc6c5)cn4)CC3)CC2)c1] |
| A230 | 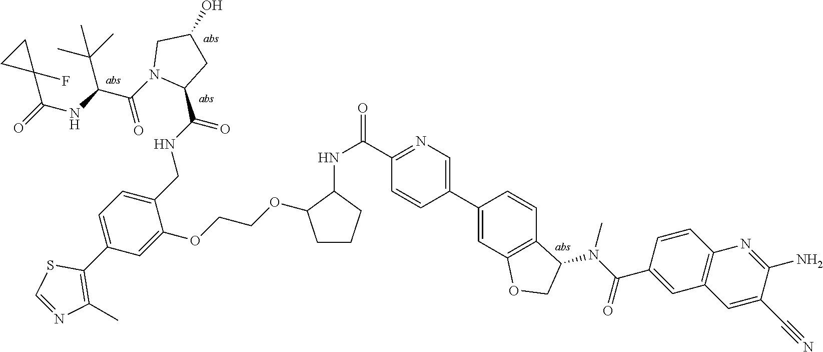 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOC2CCCC2NC(=O)c2ccc(-c3ccc4c(c3)OC[C@H]4N(C)C(=O)c3ccc4nc(N)c(C#N)cc4c3)cn2)c1] |
| A231 | 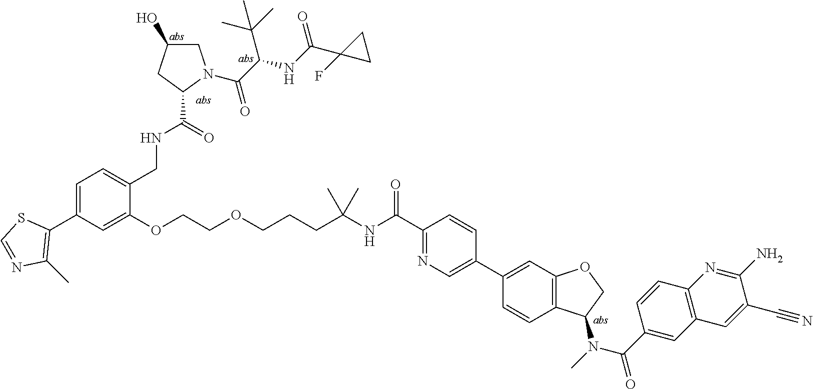 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCC(C)(C)NC(=O)c2ccc(-c3ccc4c(c3)OC[C@H]4N(C)C(=O)c3ccc4nc(N)c(C#N)cc4c3)cn2)c1] |
| A233 | 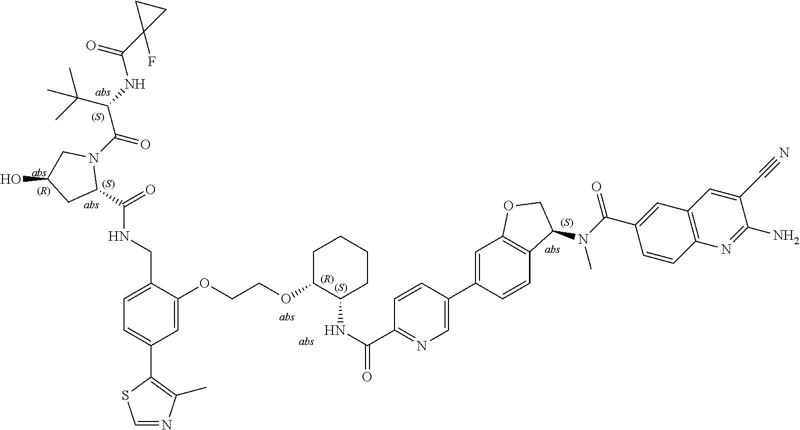 |
| A234 | 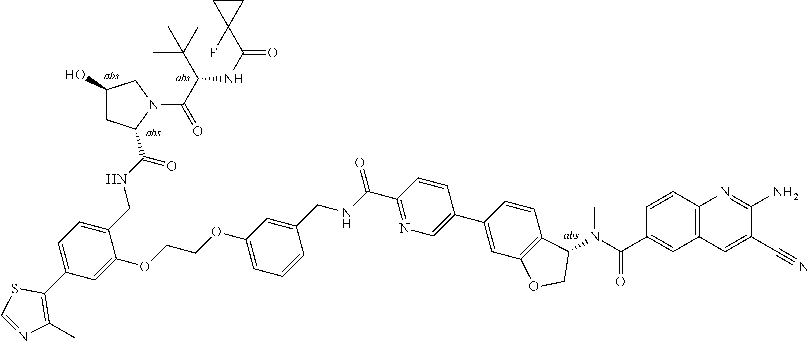 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOc2cccc(CNC(=O)c3ccc(-c4ccc5c(c4)OC[C@H]5N(C)C(=O)c4ccc5nc(N)c(C#N)cc5c4)cn3)c2)c1] |
| A235 | 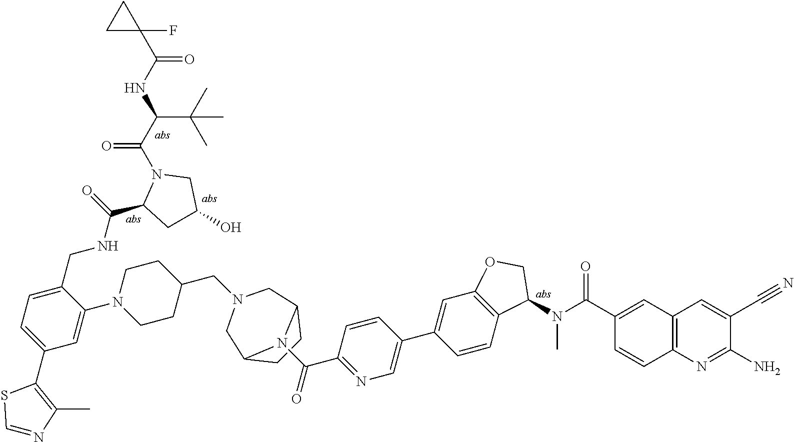 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4CCC(C3)N4C(=O)c3ccc(-c4ccc5c(c4)OC[C@H]5N(C)C(=O)c4ccc5nc(N)c(C#N)cc5c4)cn3)CC2)c1] |
| A236 | 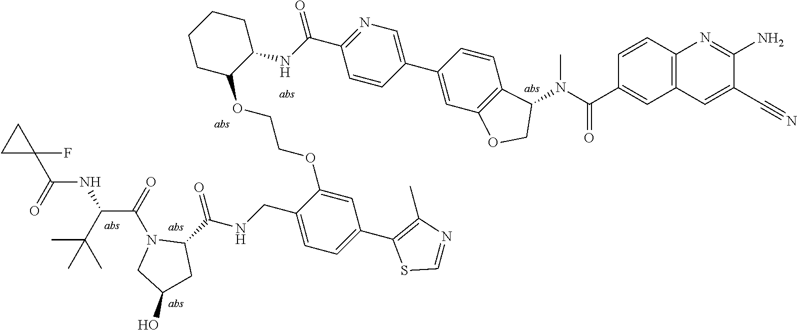 |
| A237 | 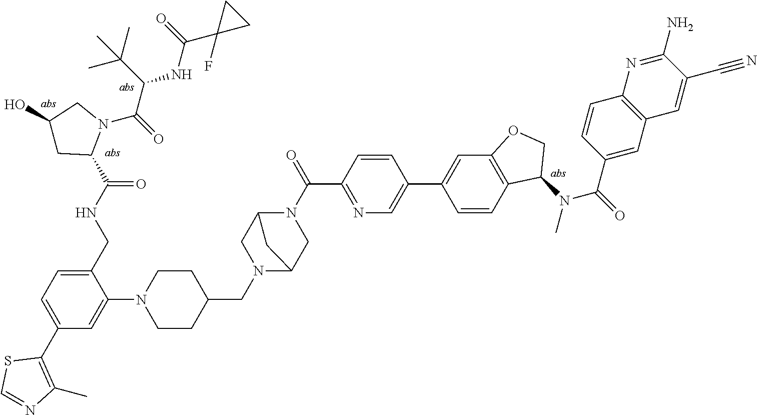 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4CC3CN4C(=O)c3ccc(-c4ccc5c(c4)OC[C@H]5N(C)C(=O)c4ccc5nc(N)c(C#N)cc5c4)cn3)CC2)c1] |
| A239 | 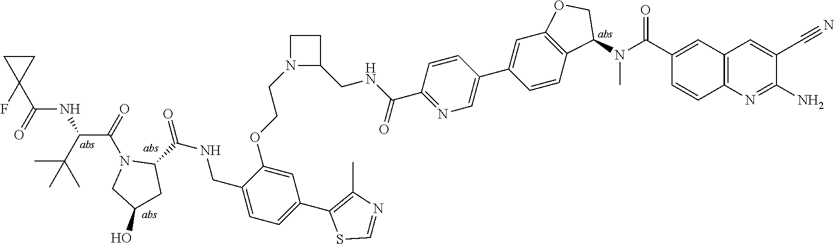 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC2CNC(=O)c2ccc(-c3ccc4c(c3)OC[C@H]4N(C)C(=O)c3ccc4nc(N)c(C#N)cc4c3)cn2)c1] |
| A240 | 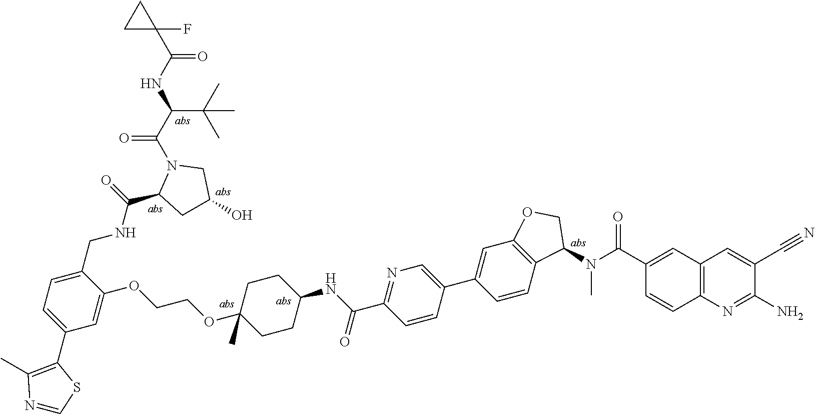 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@]2(C)CC[C@@H](NC(=O)c3ccc(-c4ccc5c(c4)OC[C@H]5N(C)C(=O)c4ccc5nc(N)c(C#N)cc5c4)cn3)CC2)c1] |
| A241 | 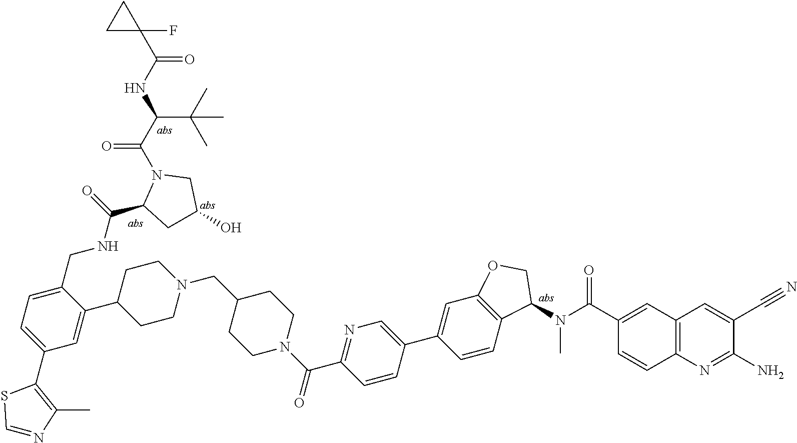 [SMILES: Cc1ncsc1-c1ccc(CNC(=O)[C@@H]2C[C@@H](O)CN2C(=O)[C@@H](NC(=O)C2(F)CC2)C(C)(C)C)c(C2CCN(CC3CCN(C(=O)c4ccc(-c5ccc6c(c5)OC[C@H]6N(C)C(=O)c5ccc6nc(N)c(C#N)cc6c5)cn4)CC3)CC2)c1] |
| A242 | 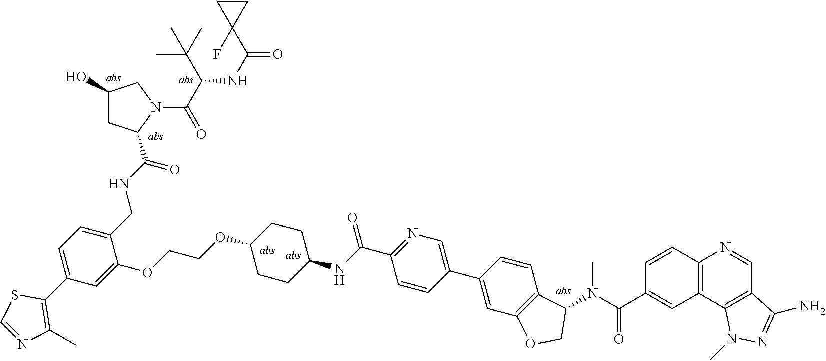 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5ncc6c(N)nn(C)c6c5c4)cn3)CC2)c1] |
| A243 | 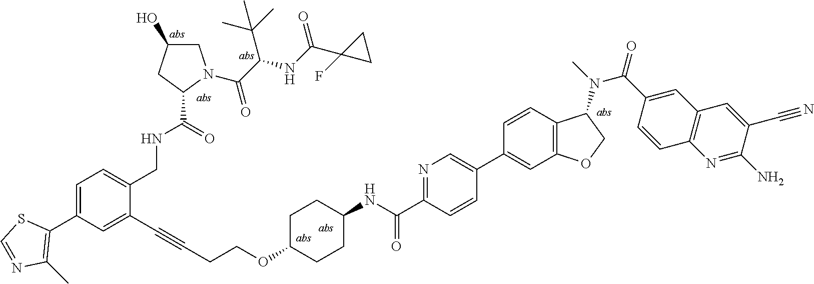 [SMILES: Cc1ncsc1-c1ccc(CNC(=O)[C@@H]2C[C@@H](O)CN2C(=O)[C@@H](NC(=O)C2(F)CC2)C(C)(C)C)c(C#CCCO[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A244 | 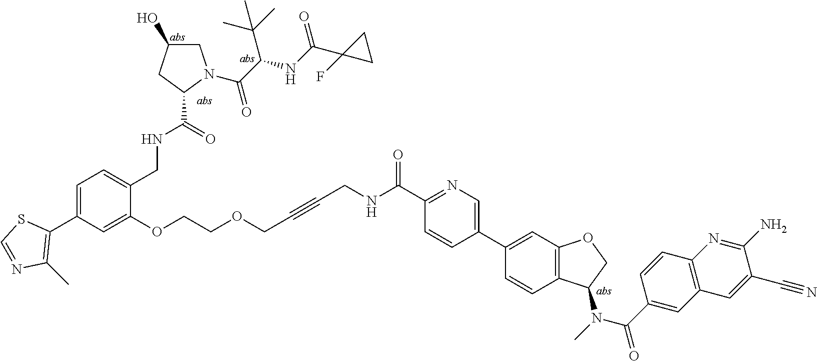 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCC#CCNC(=O)c2ccc(-c3ccc4c(c3)OC[C@H]4N(C)C(=O)c3ccc4nc(N)c(C#N)cc4c3)cn2)c1] |
| A245 | 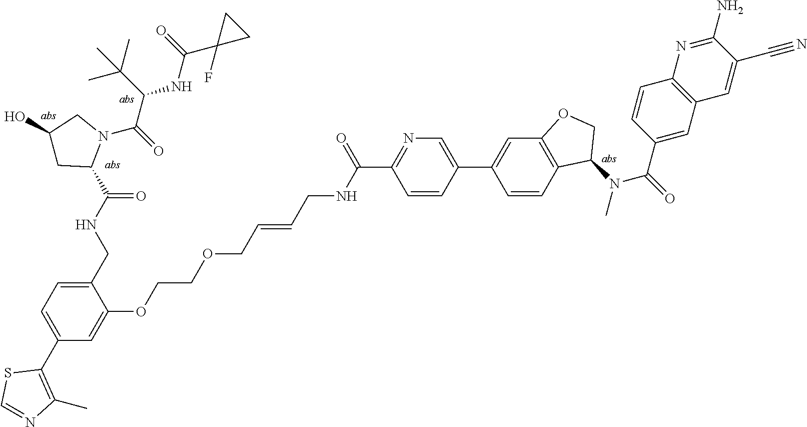 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOC/C=C/CNC(=O)c2ccc(-c3ccc4c(c3)OC[C@H]4N(C)C(=O)c3ccc4nc(N)c(C#N)cc4c3)cn2)c1] |
| A248 | 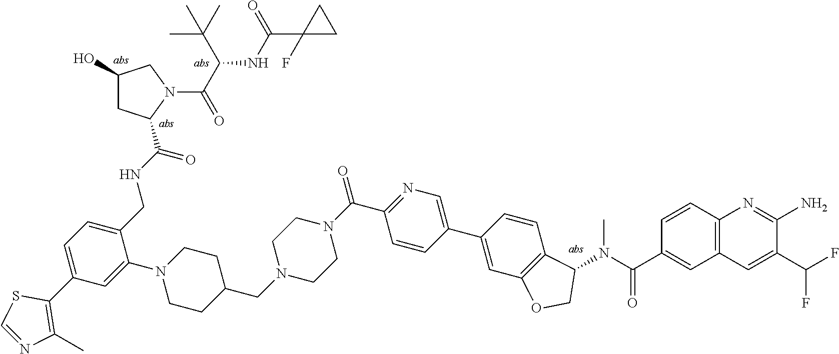 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C(F)F)cc6c5)cn4)CC3)CC2)c1] |
| A249 | 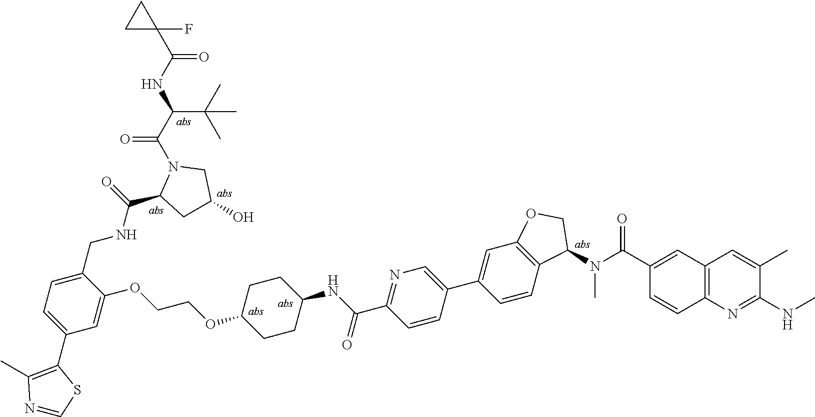 |
| A250 | 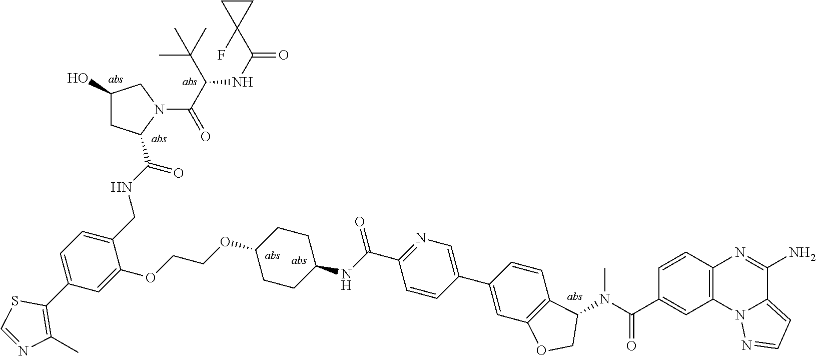 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6ccnn6c5c4)cn3)CC2)c1] |
| A251 | 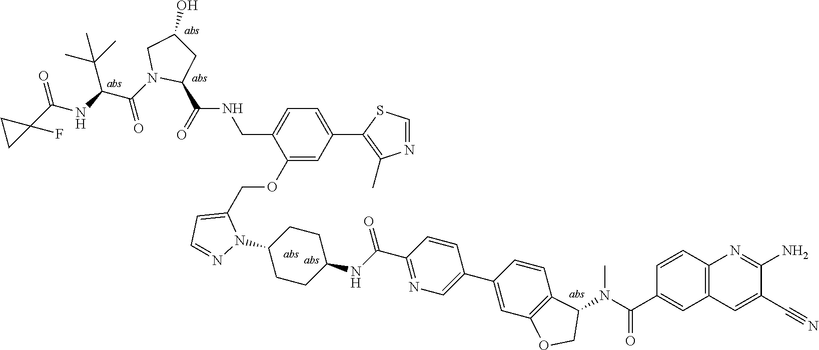 [SMILES: Cc1ncsc1-c1ccc(CNC(=O)[C@@H]2C[C@@H](O)CN2C(=O)[C@@H](NC(=O)C2(F)CC2)C(C)(C)C)c(OCc2ccnn2[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A252 | 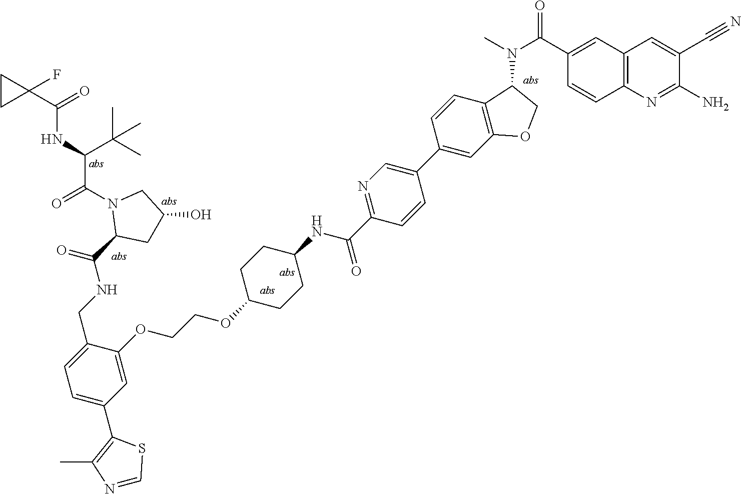 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A254 | 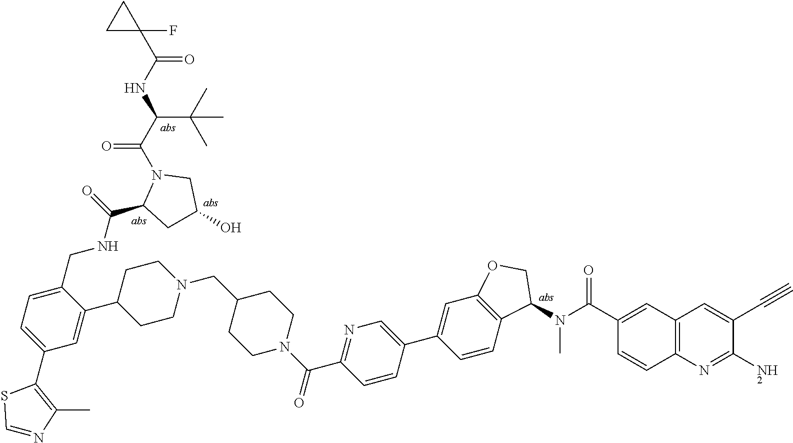 [SMILES: C#Cc1cc2cc(C(=O)N(C)[C@@H]3COc4cc(-c5ccc(C(=O)N6CCC(CN7CCC(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A255 | 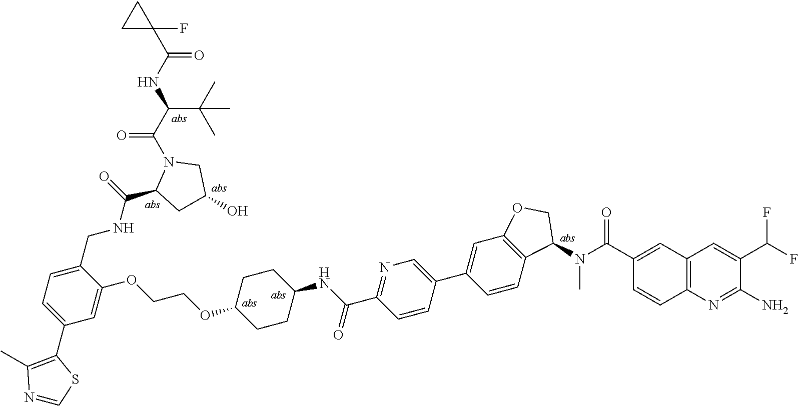 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C(F)F)cc5c4)cn3)CC2)c1] |
| A256 | 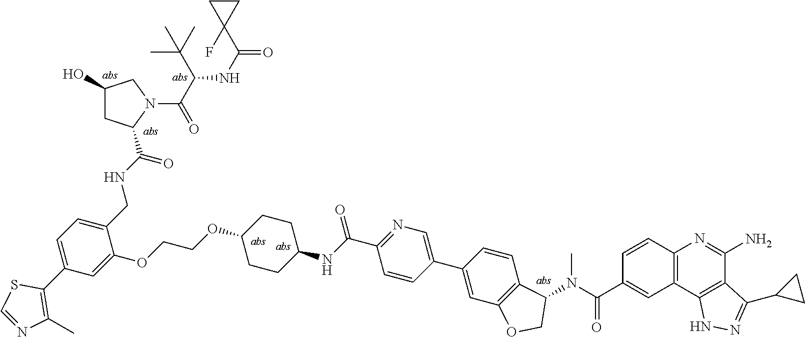 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6c(C7CC7)n[nH]c6c5c4)cn3)CC2)c1] |
| A257 | 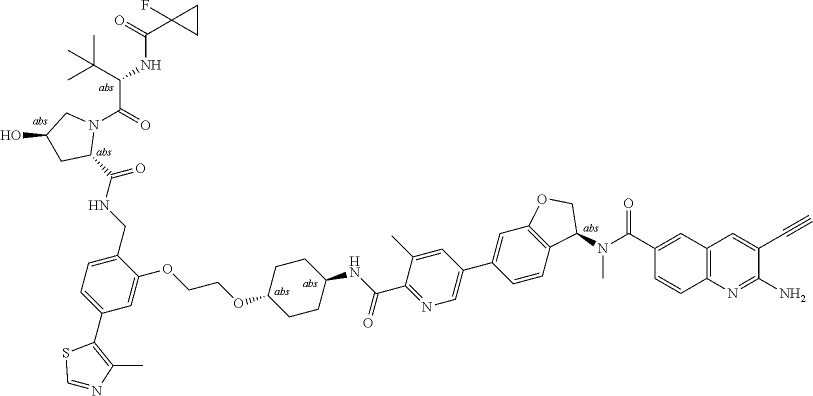 [SMILES: C#Cc1cc2cc(C(=O)N(C)[C@@H]3COc4cc(-c5cnc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)c(C)c5)ccc43)ccc2nc1N] |
| A259 | 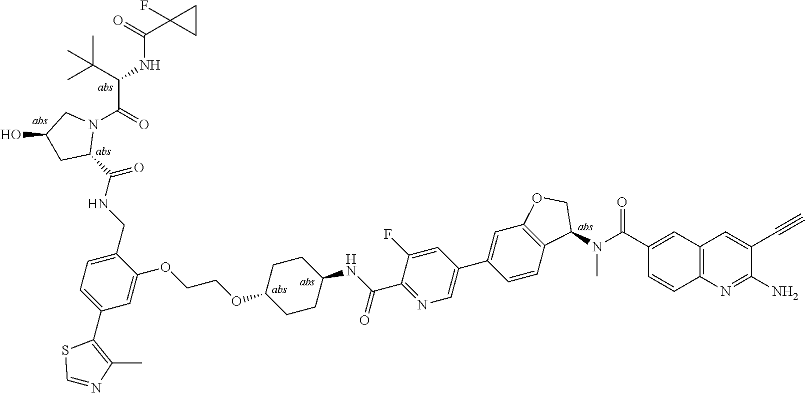 [SMILES: C#Cc1cc2cc(C(=O)N(C)[C@@H]3COc4cc(-c5cnc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)c(F)c5)ccc43)ccc2nc1N] |
| A260 | 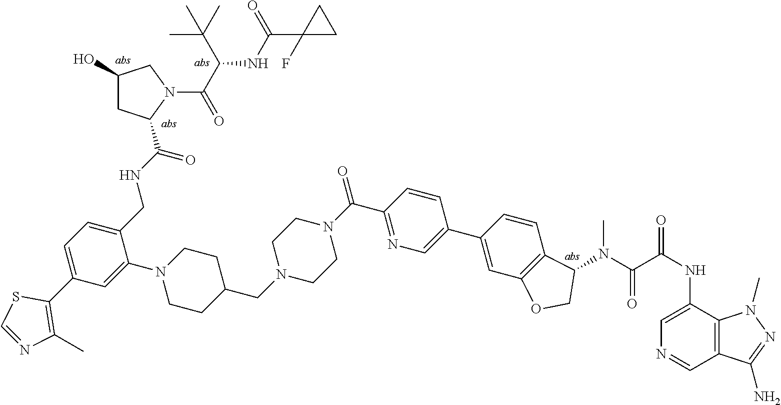 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)C(=O)Nc5cncc6c(N)nn(C)c56)cn4)CC3)CC2)c1] |
| A262 | 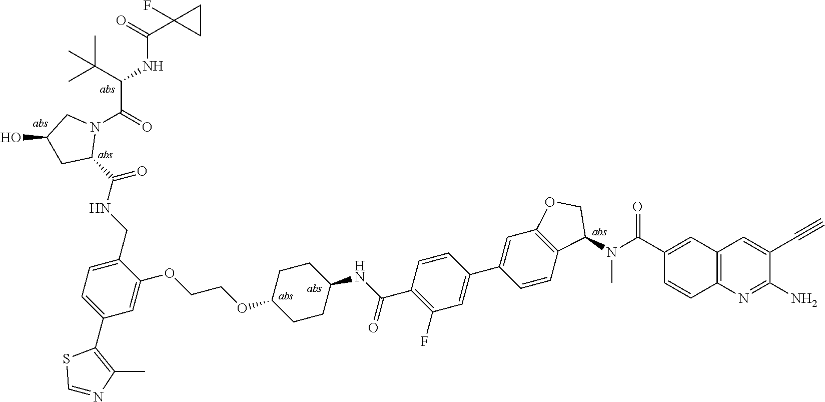 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)c(F)c5)ccc43)ccc2nc1N] |
| A263 | 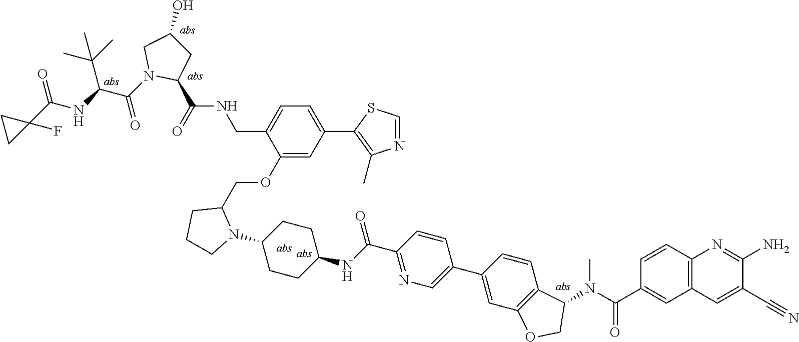 [SMILES: Cc1ncsc1-c1ccc(CNC(=O)[C@@H]2C[C@@H](O)CN2C(=O)[C@@H](NC(=O)C2(F)CC2)C(C)(C)C)c(OCC2CCCN2[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A264 | 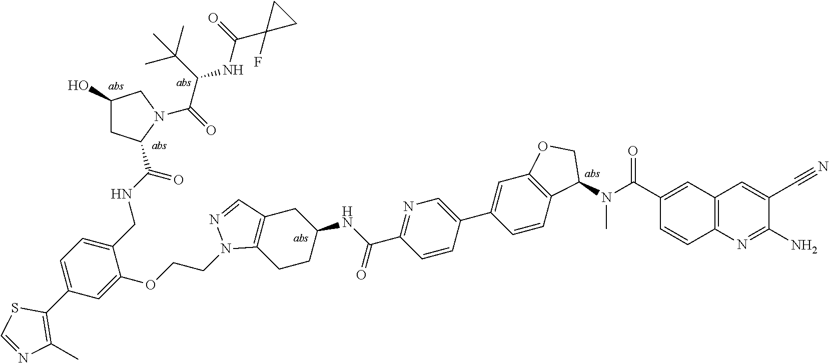 [SMILES: Cc1ncsc1-c1ccc(CNC(=O)[C@@H]2C[C@@H](O)CN2C(=O)[C@@H](NC(=O)C2(F)CC2)C(C)(C)C)c(OCCn2ncc3c2CC[C@H](NC(=O)c2ccc(-c4ccc5c(c4)OC[C@H]5N(C)C(=O)c4ccc5nc(N)c(C#N)cc5c4)cn2)C3)c1] |
| A265 | 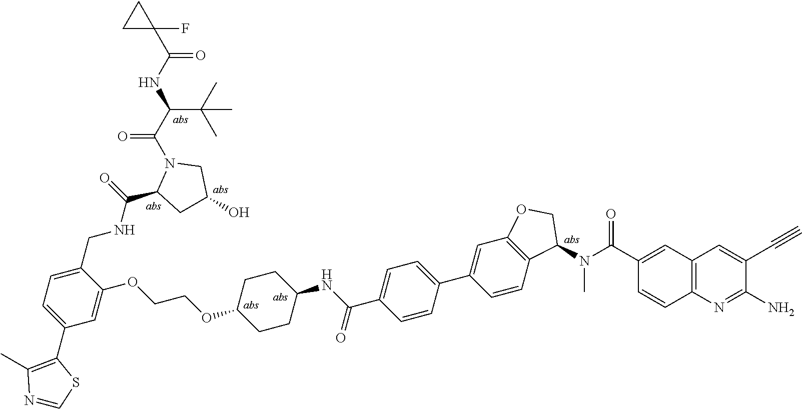 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)cc5)ccc43)ccc2nc1N] |
| A266 | 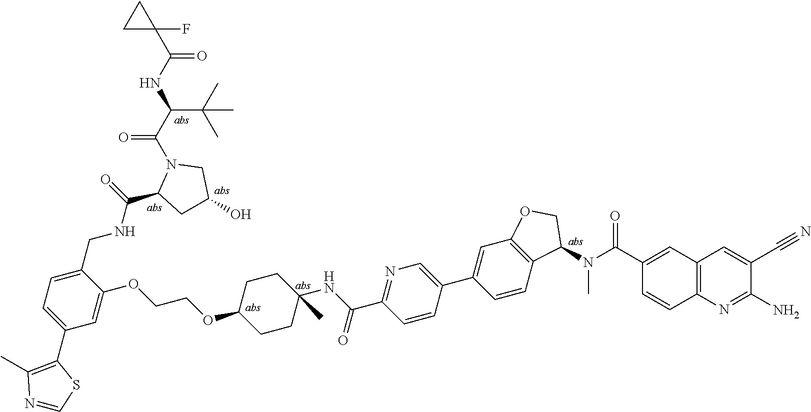 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@](C)(NC(=O)c3ccc(-c4ccc5c(c4)OC[C@H]5N(C)C(=O)c4ccc5nc(N)c(C#N)cc5c4)cn3)CC2)c1] |
| A267 | 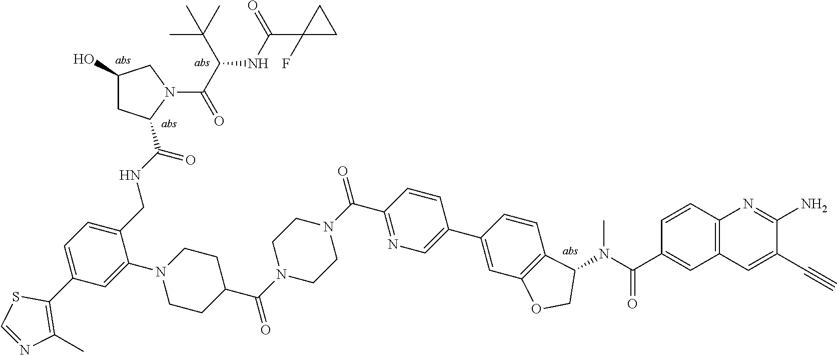 [SMILES: C#Cc1cc2cc(C(=O)N(C)[C@@H]3COc4cc(-c5ccc(C(=O)N6CCN(C(=O)C7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A268 | 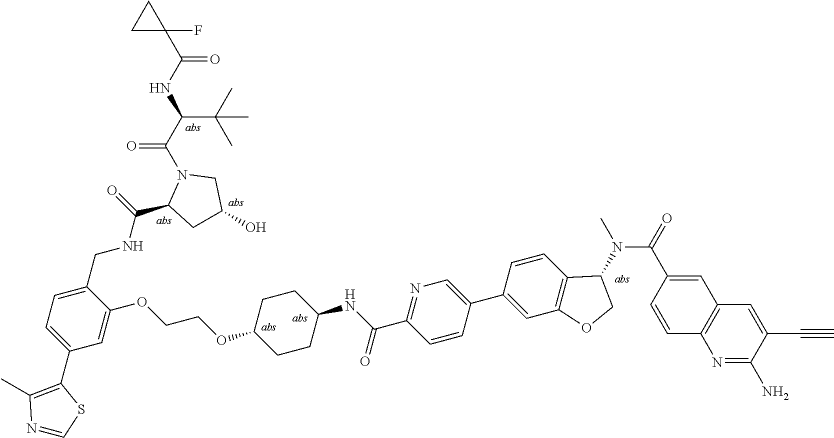 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A269 | 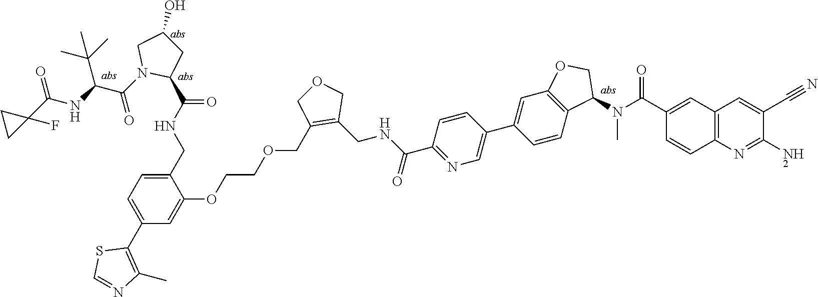 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCC2=C(CNC(=O)c3ccc(-c4ccc5c(c4)OC[C@H]5N(C)C(=O)c4ccc5nc(N)c(C#N)cc5c4)cn3)COC2)c1] |
| A270 | 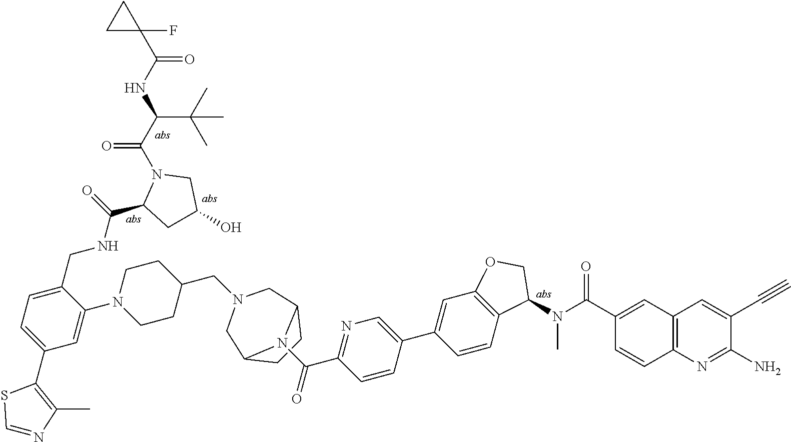 [SMILES: C#Cc1cc2cc(C(=O)N(C)[C@@H]3COc4cc(-c5ccc(C(=O)N6C7CCC6CN(CC6CCN(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A271 | 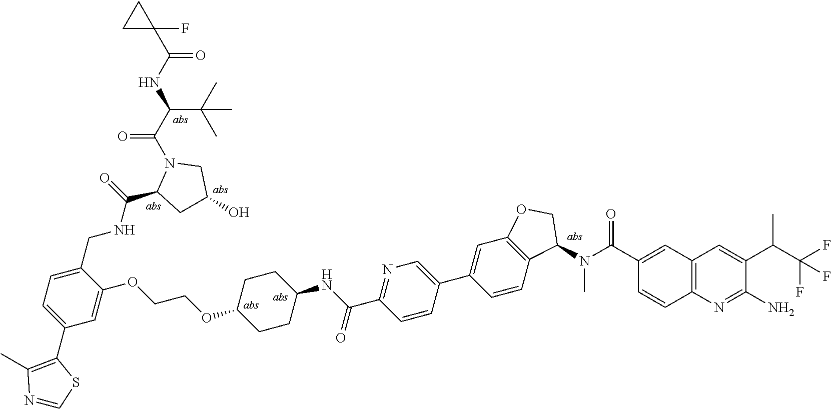 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C(C)C(F)(F)F)cc5c4)cn3)CC2)c1] |
| A272 | 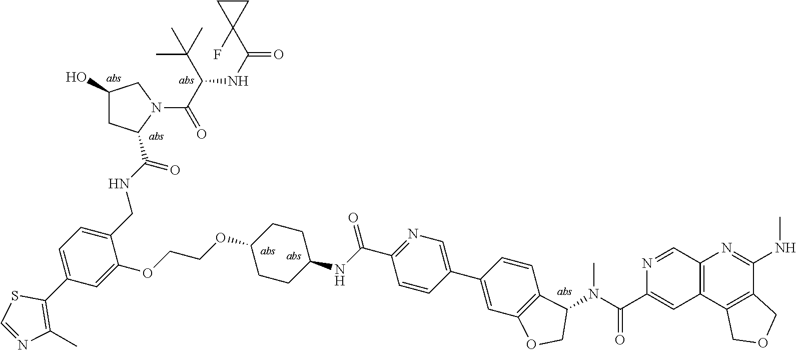 [SMILES: CNc1nc2cnc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2c2c1COC2] |
| A274 | 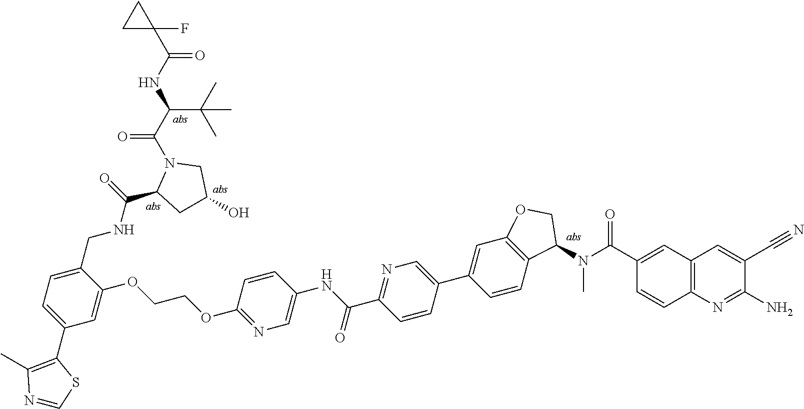 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOc2ccc(NC(=O)c3ccc(-c4ccc5c(c4)OC[C@H]5N(C)C(=O)c4ccc5nc(N)c(C#N)cc5c4)cn3)cn2)c1] |
| A275 | 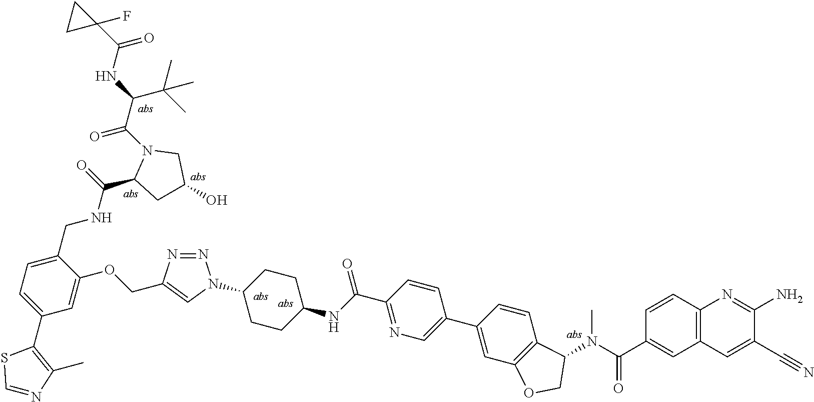 [SMILES: Cc1ncsc1-c1ccc(CNC(=O)[C@@H]2C[C@@H](O)CN2C(=O)[C@@H](NC(=O)C2(F)CC2)C(C)(C)C)c(OCc2cn([C@H]3CC[C@H](NC(=O)c4ccc(-c5ccc6c(c5)OC[C@H]6N(C)C(=O)c5ccc6nc(N)c(C#N)cc6c5)cn4)CC3)nn2)c1] |
| A276 | 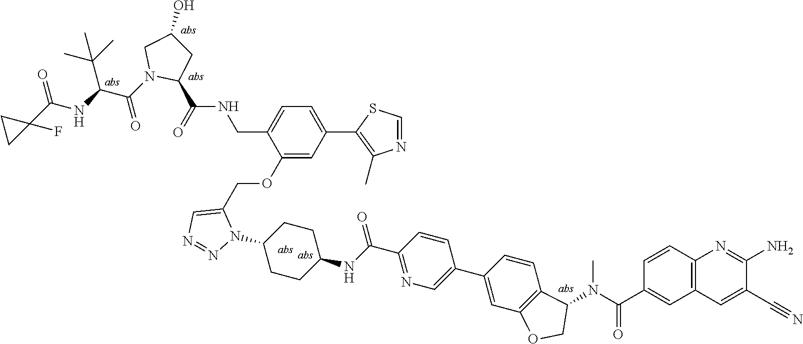 [SMILES: Cc1ncsc1-c1ccc(CNC(=O)[C@@H]2C[C@@H](O)CN2C(=O)[C@@H](NC(=O)C2(F)CC2)C(C)(C)C)c(OCc2cnnn2[C@H]2CC[C@H](NC(=O)c3ccc(-c4ccc5c(c4)OC[C@H]5N(C)C(=O)c4ccc5nc(N)c(C#N)cc5c4)cn3)CC2)c1] |
| A278 | 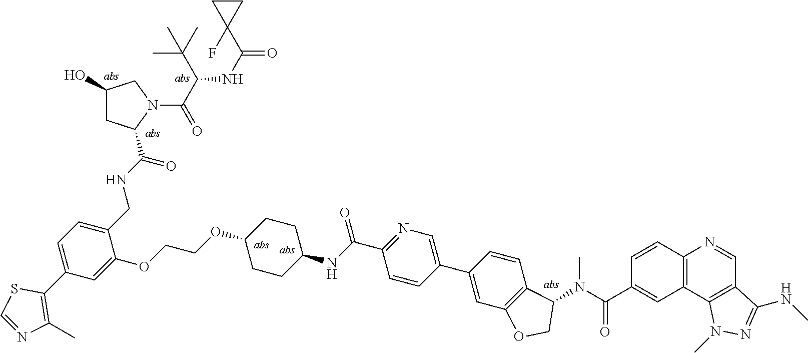 [SMILES: CNc1nn(C)c2c1cnc1ccc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc12] |
| A279 | 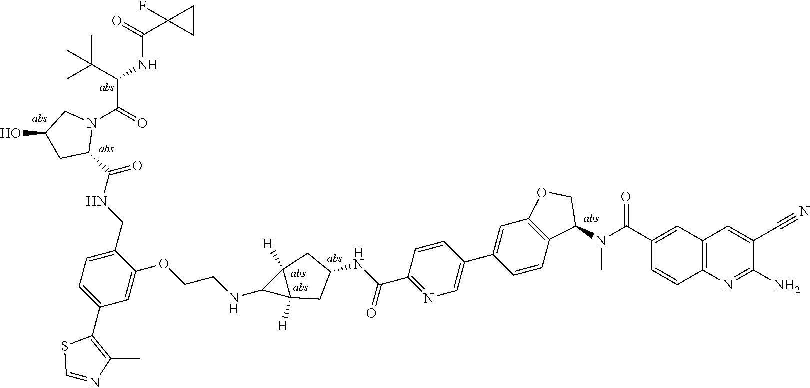 [SMILES: Cc1ncsc1-c1ccc(CNC(=O)[C@@H]2C[C@@H](O)CN2C(=O)[C@@H](NC(=O)C2(F)CC2)C(C)(C)C)c(OCCNC2[C@H]3C[C@H](NC(=O)c4ccc(-c5ccc6c(c5)OC[C@H]6N(C)C(=O)c5ccc6nc(N)c(C#N)cc6c5)cn4)C[C@@H]23)c1] |
| A280 | 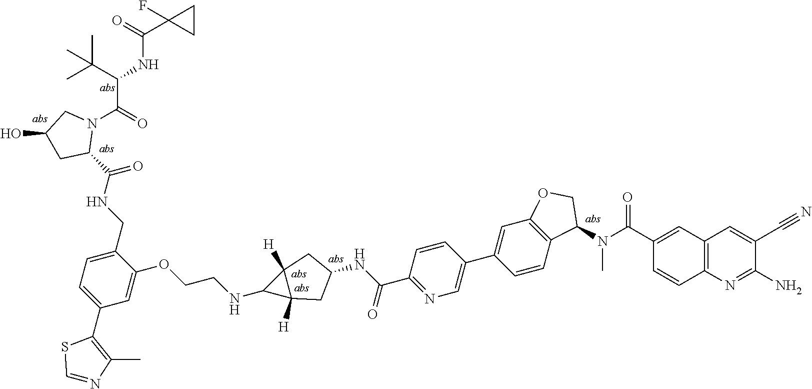 [SMILES: Cc1ncsc1-c1ccc(CNC(=O)[C@@H]2C[C@@H](O)CN2C(=O)[C@@H](NC(=O)C2(F)CC2)C(C)(C)C)c(OCCNC2[C@H]3C[C@@H](NC(=O)c4ccc(-c5ccc6c(c5)OC[C@H]6N(C)C(=O)c5ccc6nc(N)c(C#N)cc6c5)cn4)C[C@@H]23)c1] |
| A281 | 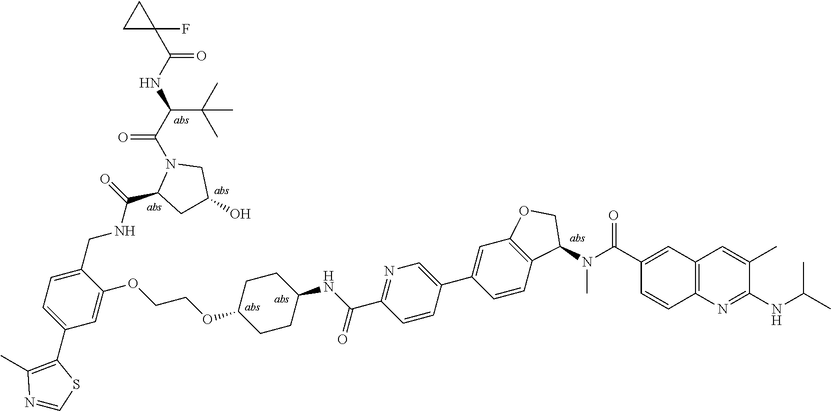 [SMILES: Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1NC(C)C] |
| A282 | 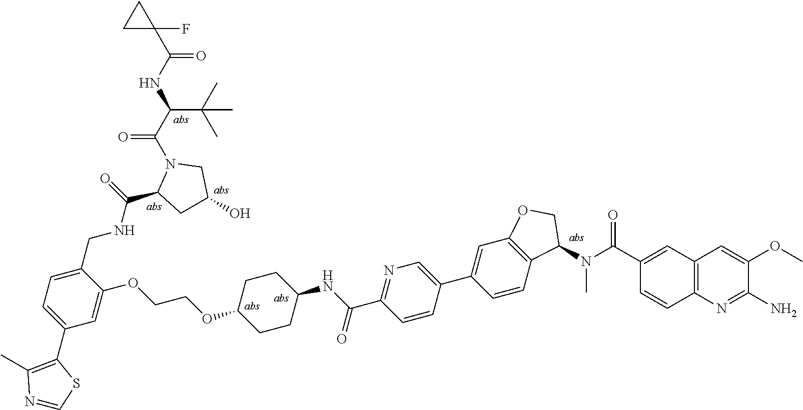 |
| A283 | 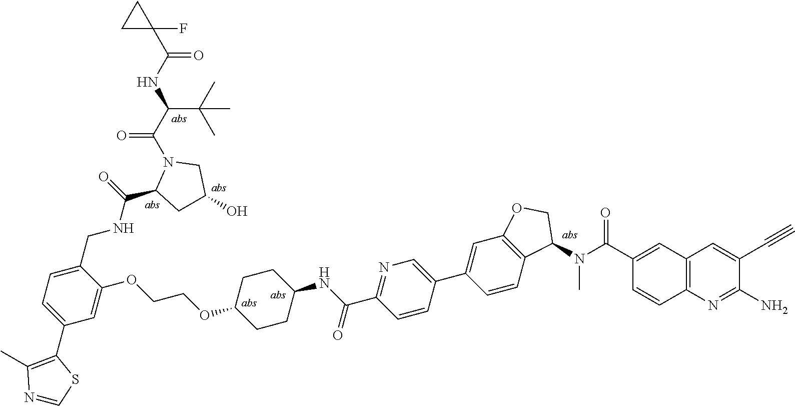 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A284 | 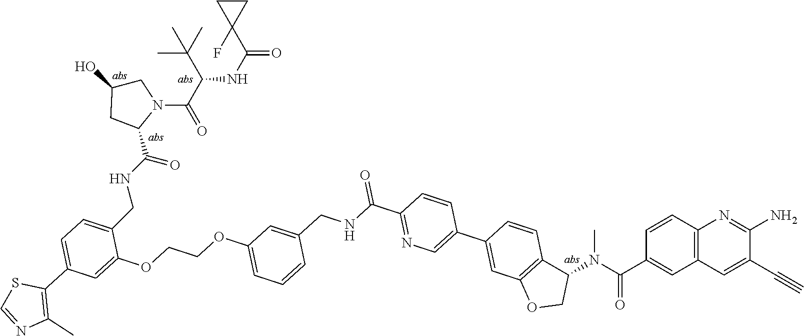 [SMILES: C#Cc1cc2cc(C(=O)N(C)[C@@H]3COc4cc(-c5ccc(C(=O)NCc6cccc(OCCOc7cc(-c8scnc8C)ccc7CNC(=O)[C@@H]7C[C@@H](O)CN7C(=O)[C@@H](NC(=O)C7(F)CC7)C(C)(C)C)c6)nc5)ccc43)ccc2nc1N] |
| A285 | 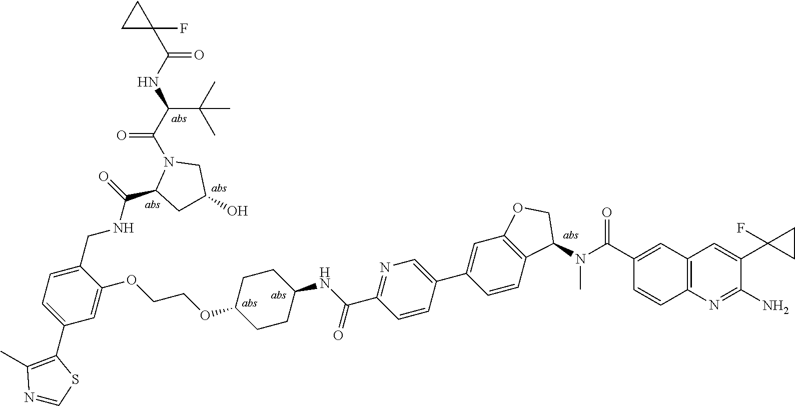 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6(F)CC6)cc5c4)cn3)CC2)c1] |
| A286 | 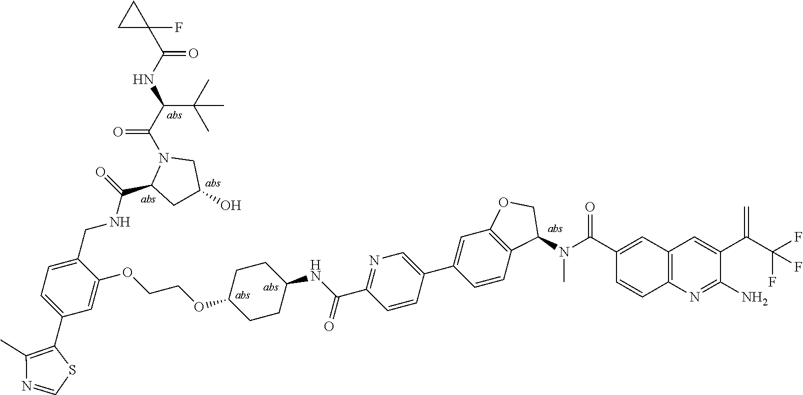 [SMILES: C=C(c1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N)C(F)(F)F] |
| A287 | 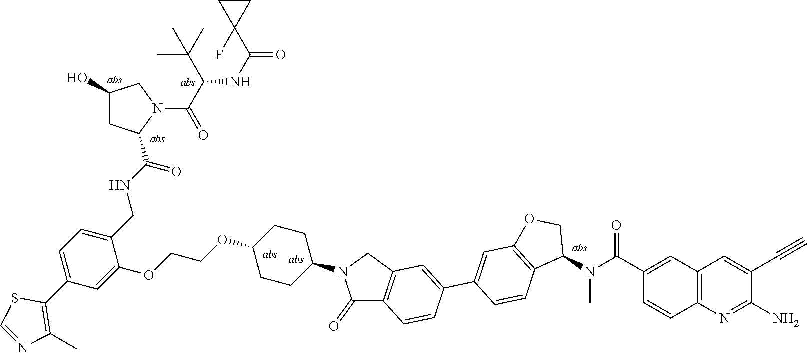 [SMILES: C#Cc1cc2cc(C(=O)N(C)[C@@H]3COc4cc(-c5ccc6c(c5)CN([C@H]5CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC5)C6=O)ccc43)ccc2nc1N] |
| A288 | 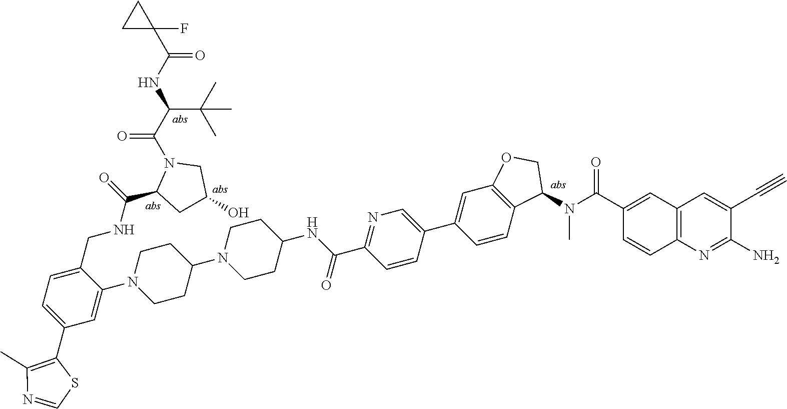 [SMILES: C#Cc1cc2cc(C(=O)N(C)[C@@H]3COc4cc(-c5ccc(C(=O)NC6CCN(C7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A289 | 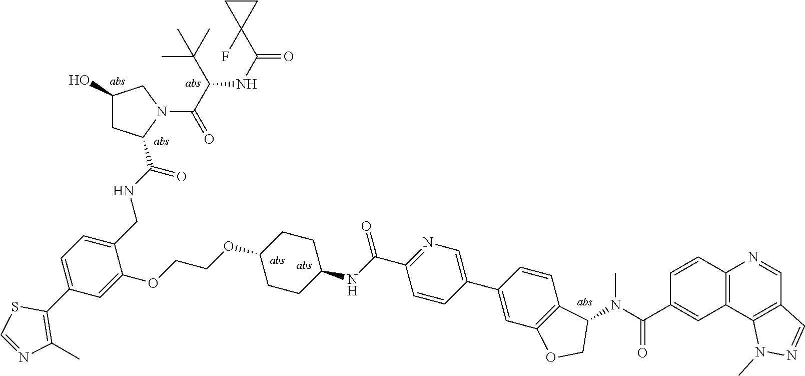 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5ncc6cnn(C)c6c5c4)cn3)CC2)c1] |
| A290 | 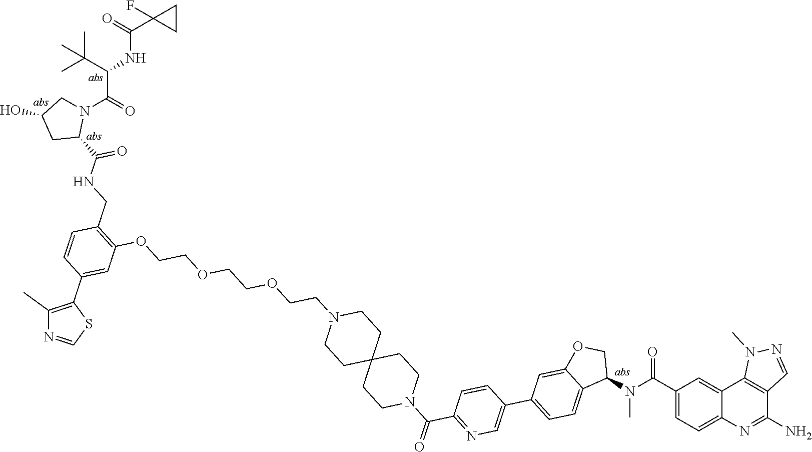 [SMILES: Cc1ncsc1-c1ccc(CNC(=O)[C@@H]2C[C@H](O)CN2C(=O)[C@@H](NC(=O)C2(F)CC2)C(C)(C)C)c(OCCOCCOCCN2CCC3(CC2)CCN(C(=O)c2ccc(-c4ccc5c(c4)OC[C@H]5N(C)C(=O)c4ccc5nc(N)c6cnn(C)c6c5c4)cn2)CC3)c1] |
| A291 | 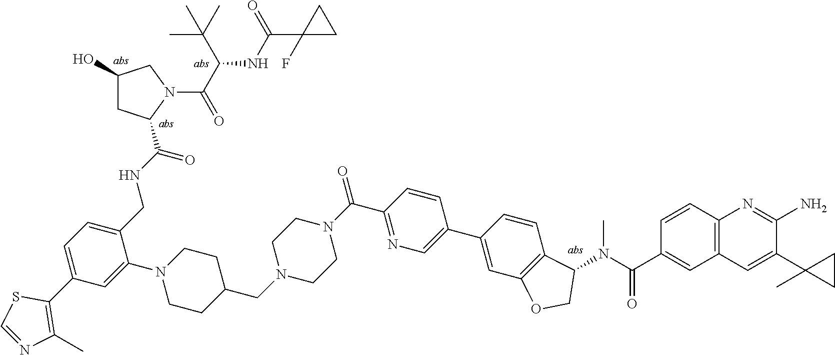 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C7(C)CC7)cc6c5)cn4)CC3)CC2)c1] |
| A292 | 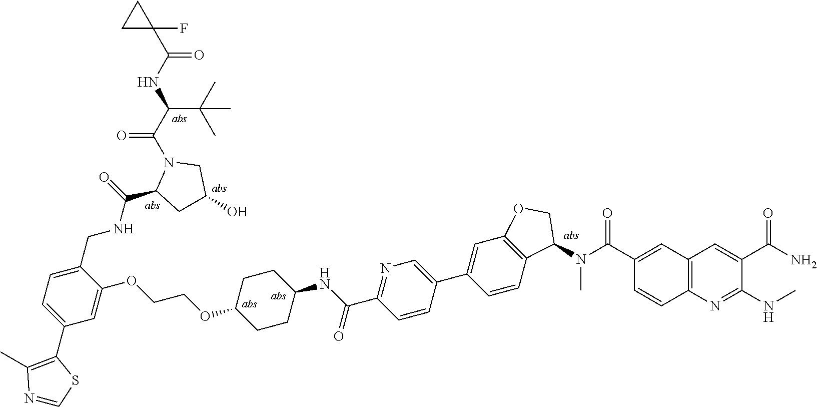 |
| A293 | 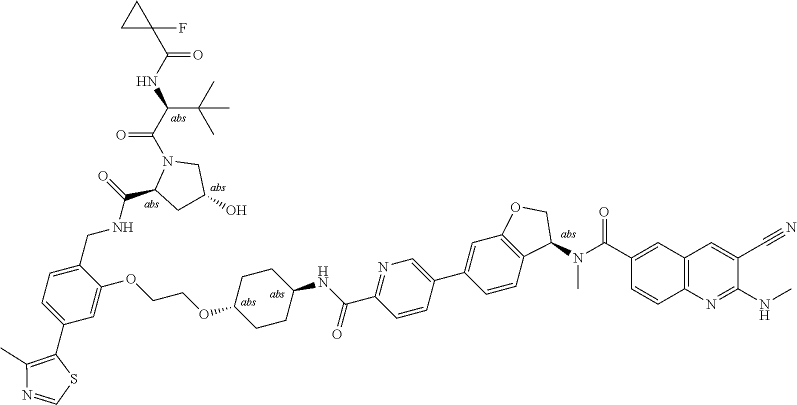 |
| A294 | 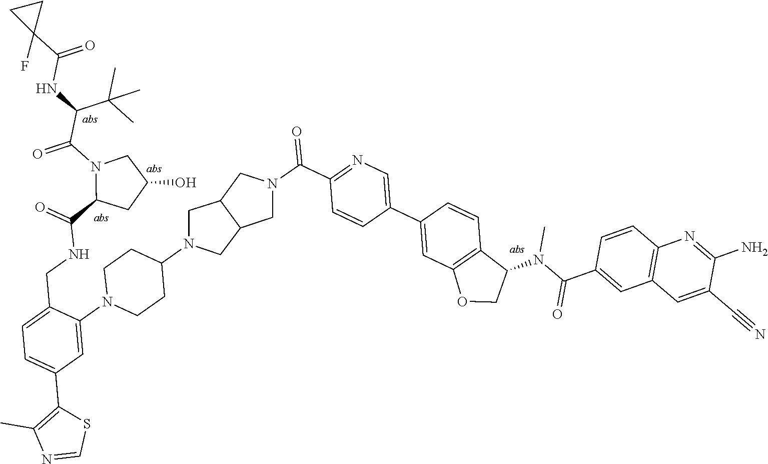 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(N3CC4CN(C(=O)c5ccc(-c6ccc7c(c6)OC[C@H]7N(C)C(=O)c6ccc7nc(N)c(C#N)cc7c6)cn5)CC4C3)CC2)c1] |
| A295 | 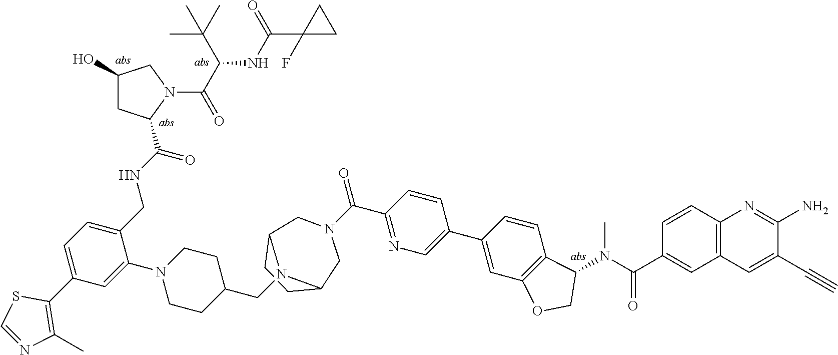 [SMILES: C#Cc1cc2cc(C(=O)N(C)[C@@H]3COc4cc(-c5ccc(C(=O)N6CC7CCC(C6)N7CC6CCN(c7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A297 | 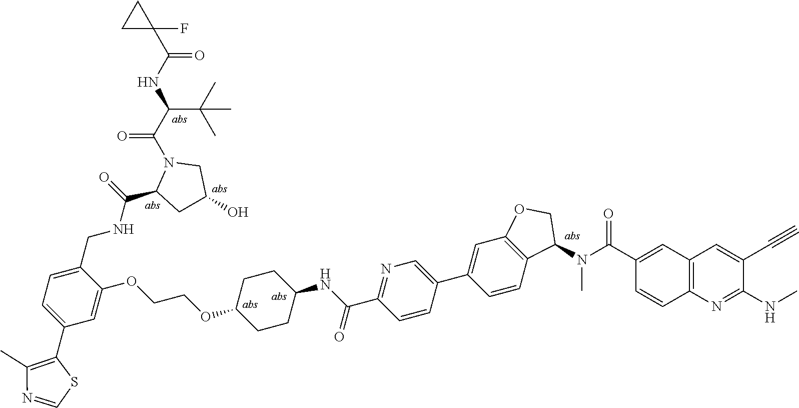 |
| A298 | 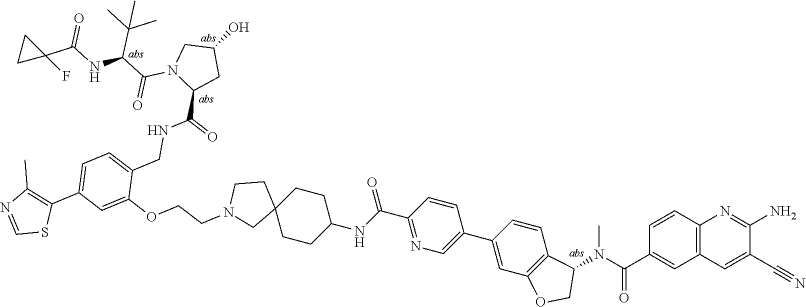 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC3(CCC(NC(=O)c4ccc(-c5ccc6c(c5)OC[C@H]6N(C)C(=O)c5ccc6nc(N)c(C#N)cc6c5)cn4)CC3)C2)c1] |
| A299 | 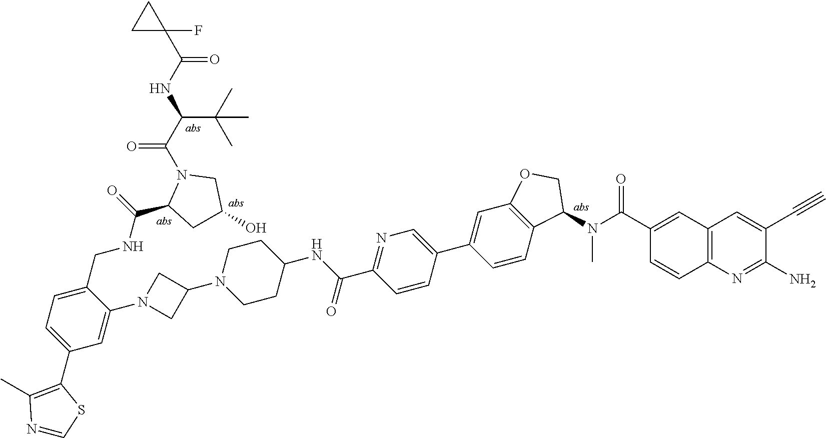 [SMILES: C#Cc1cc2cc(C(=O)N(C)[C@@H]3COc4cc(-c5ccc(C(=O)NC6CCN(C7CN(c8cc(-c9scnc9C)ccc8CNC(=O)[C@@H]8C[C@@H](O)CN8C(=O)[C@@H](NC(=O)C8(F)CC8)C(C)(C)C)C7)CC6)nc5)ccc43)ccc2nc1N] |
| A301 | 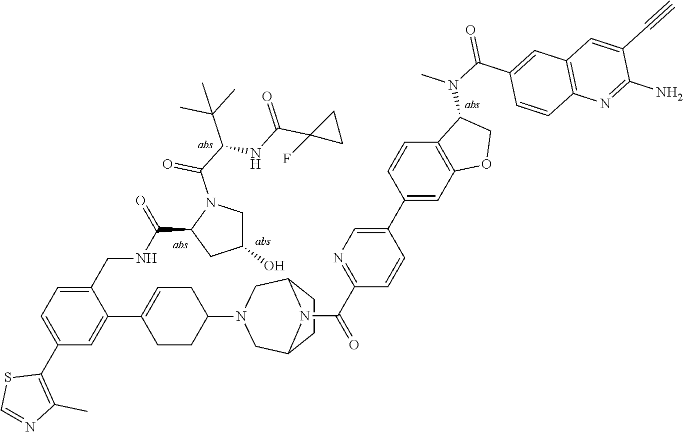 [SMILES: C#Cc1cc2cc(C(=O)N(C)[C@@H]3COc4cc(-c5ccc(C(=O)N6C7CCC6CN(C6CC=C(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A303 | 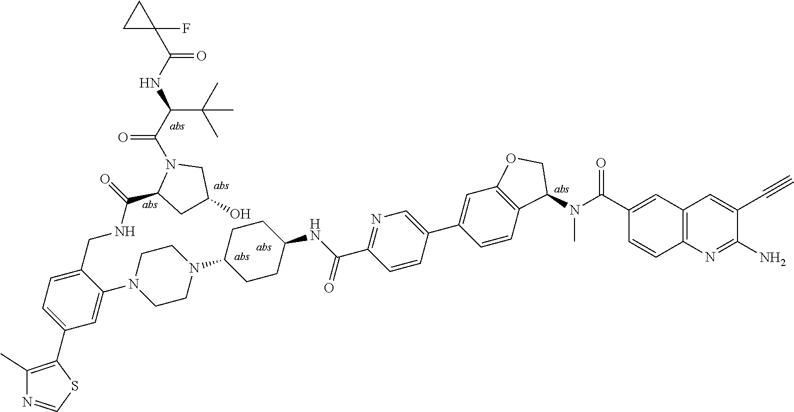 [SMILES: C#Cc1cc2cc(C(=O)N(C)[C@@H]3COc4cc(-c5ccc(C(=O)N[C@H]6CC[C@H](N7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A304 | 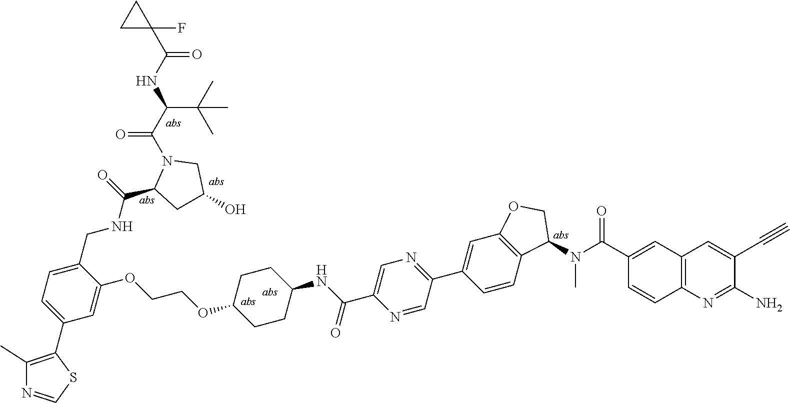 [SMILES: C#Cc1cc2cc(C(=O)N(C)[C@@H]3COc4cc(-c5cnc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)cn5)ccc43)ccc2nc1N] |
| A305 | 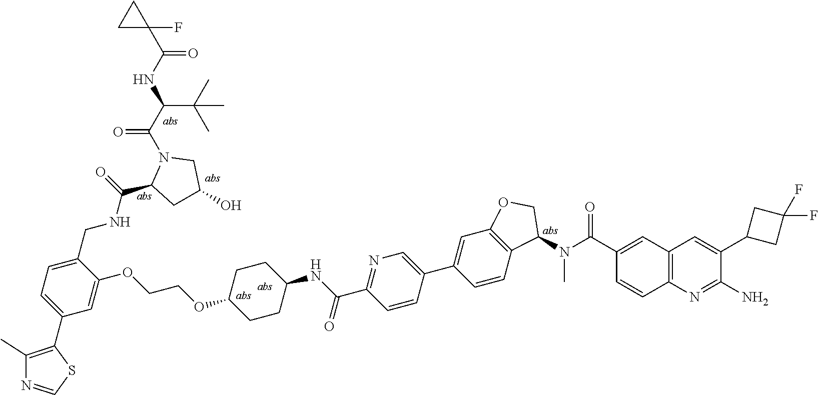 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CC(F)(F)C6)cc5c4)cn3)CC2)c1] |
| A306 | 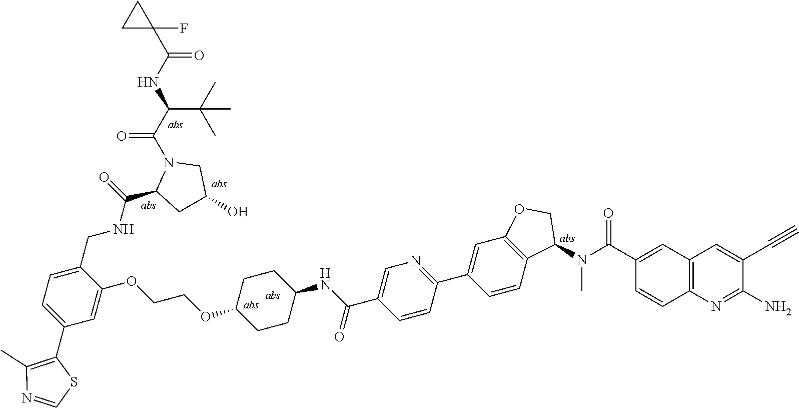 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)cn5)ccc43)ccc2nc1N] |
| A307 | 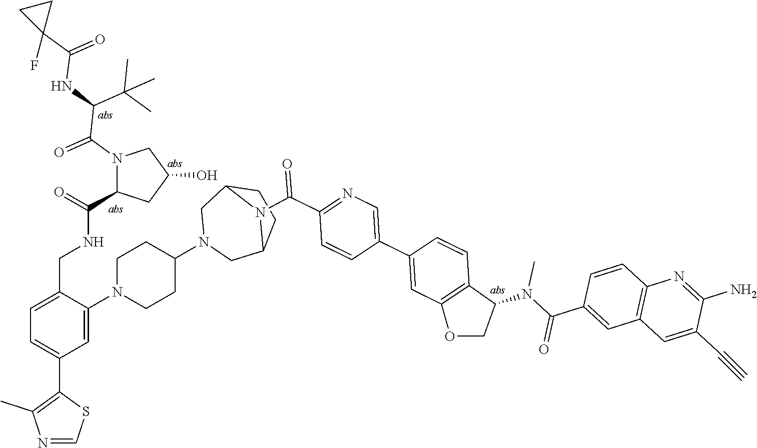 [SMILES: C#Cc1cc2cc(C(=O)N(C)[C@@H]3COc4cc(-c5ccc(C(=O)N6C7CCC6CN(C6CCN(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A308 | 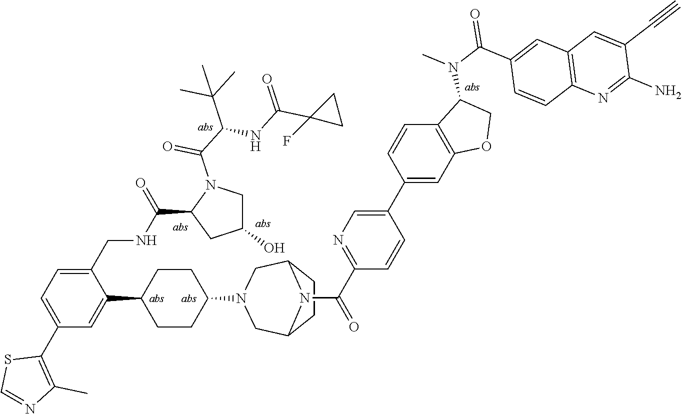 [SMILES: C#Cc1cc2cc(C(=O)N(C)[C@@H]3COc4cc(-c5ccc(C(=O)N6C7CCC6CN([C@H]6CC[C@H](c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A309 | 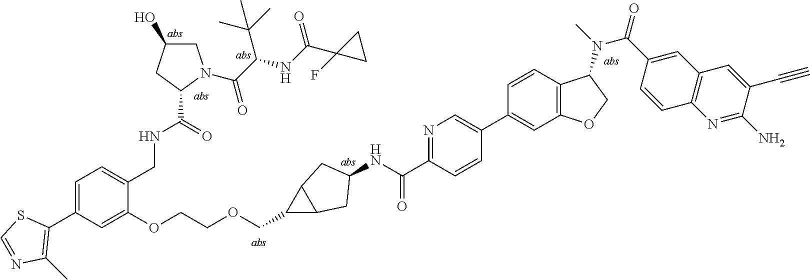 |
| A310 | 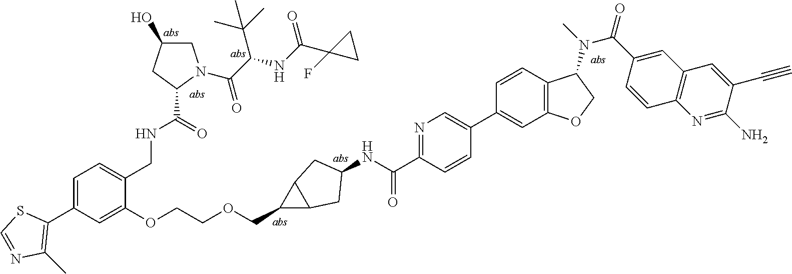 [SMILES: C#Cc1cc2cc(C(=O)N(C)[C@@H]3COc4cc(-c5ccc(C(=O)N[C@H]6CC7C(C6)[C@@H]7COCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N] |
| A311 | 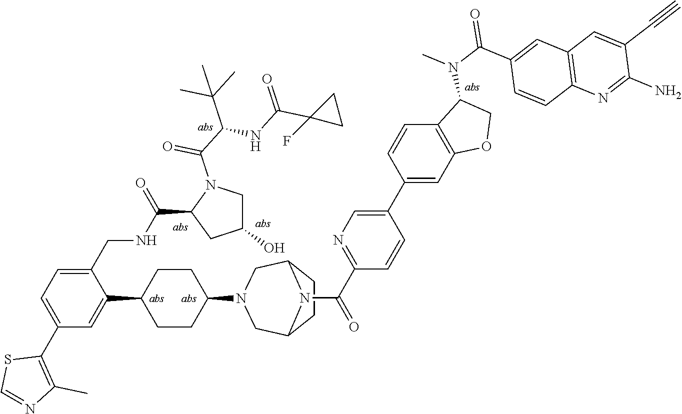 [SMILES: C#Cc1cc2cc(C(=O)N(C)[C@@H]3COc4cc(-c5ccc(C(=O)N6C7CCC6CN([C@H]6CC[C@@H](c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A312 | 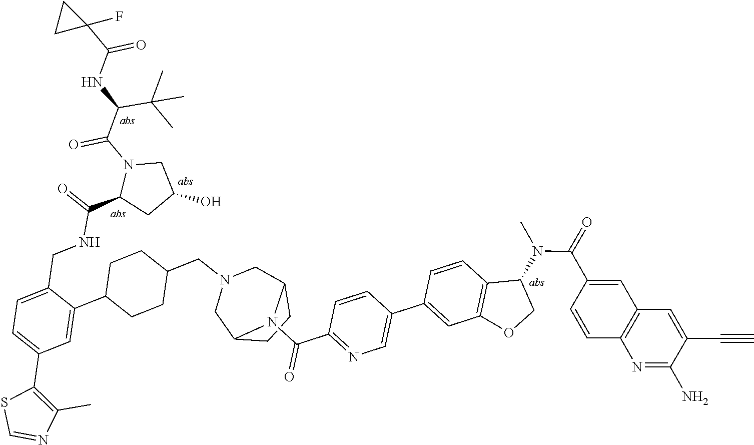 [SMILES: C#Cc1cc2cc(C(=O)N(C)[C@@H]3COc4cc(-c5ccc(C(=O)N6C7CCC6CN(CC6CCC(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A313 | 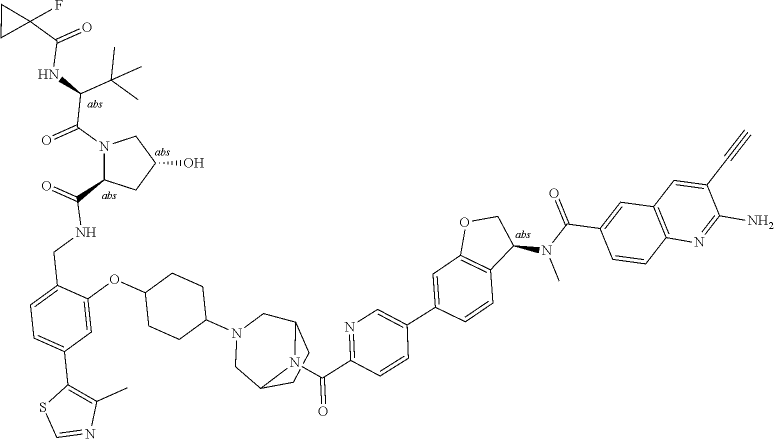 [SMILES: C#Cc1cc2cc(C(=O)N(C)[C@@H]3COc4cc(-c5ccc(C(=O)N6C7CCC6CN(C6CCC(Oc8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A314 | 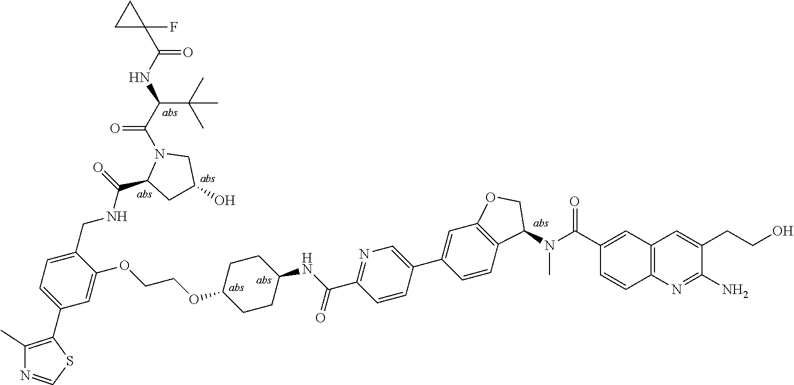 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(CCO)cc5c4)cn3)CC2)c1] |
| A315 | 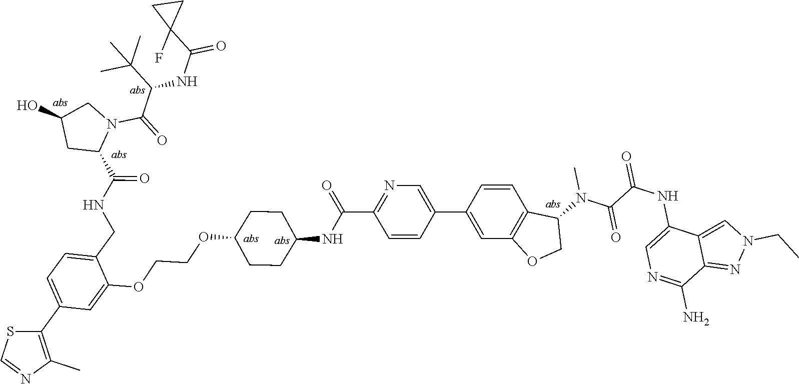 [SMILES: CCn1cc2c(NC(=O)C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cnc(N)c2n1] |
| A316 | 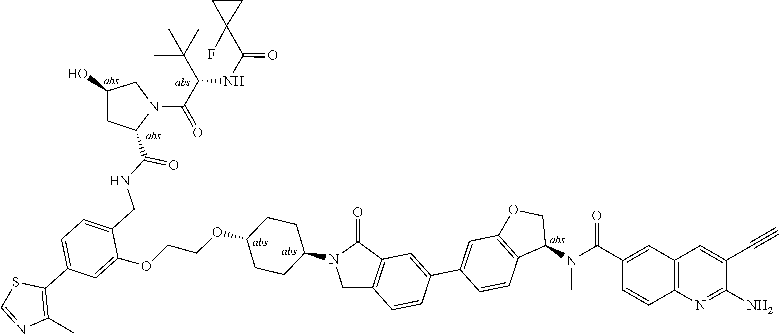 [SMILES: C#Cc1cc2cc(C(=O)N(C)[C@@H]3COc4cc(-c5ccc6c(c5)C(=O)N([C@H]5CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC5)C6)ccc43)ccc2nc1N] |
| A318 | 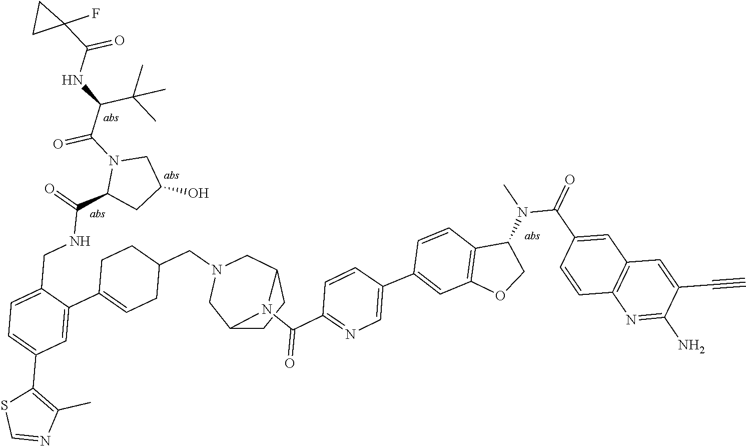 [SMILES: C#Cc1cc2cc(C(=O)N(C)[C@@H]3COc4cc(-c5ccc(C(=O)N6C7CCC6CN(CC6CC=C(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A319 | 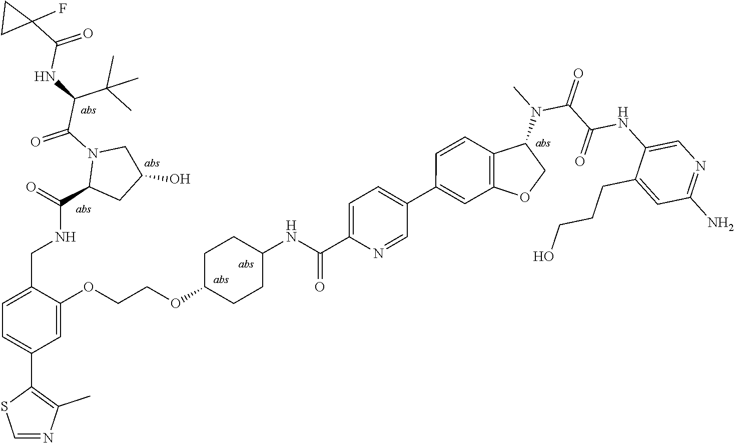 |
| A321 | 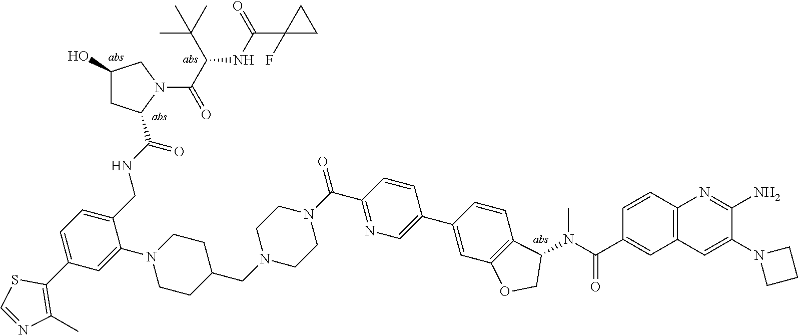 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(N7CCC7)cc6c5)cn4)CC3)CC2)c1] |
| A323 | 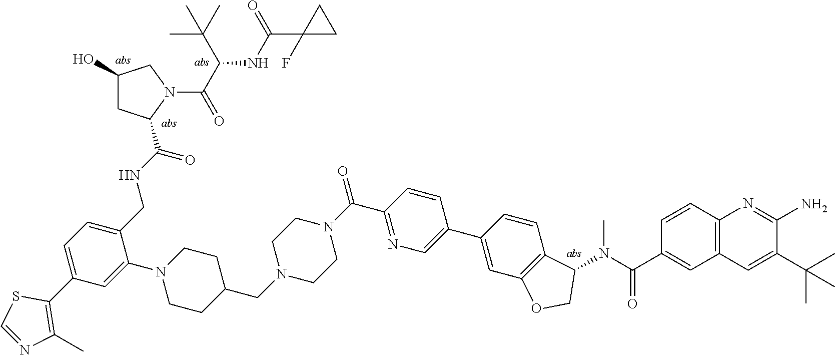 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c(C(C)(C)C)cc6c5)cn4)CC3)CC2)c1] |
| A324 | 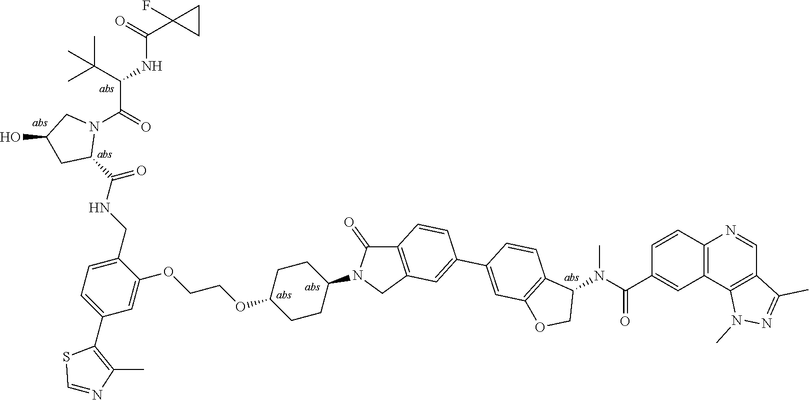 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](N3Cc4cc(-c5ccc6c(c5)OC[C@H]6N(C)C(=O)c5ccc6ncc7c(C)nn(C)c7c6c5)ccc4C3=O)CC2)c1] |
| A325 | 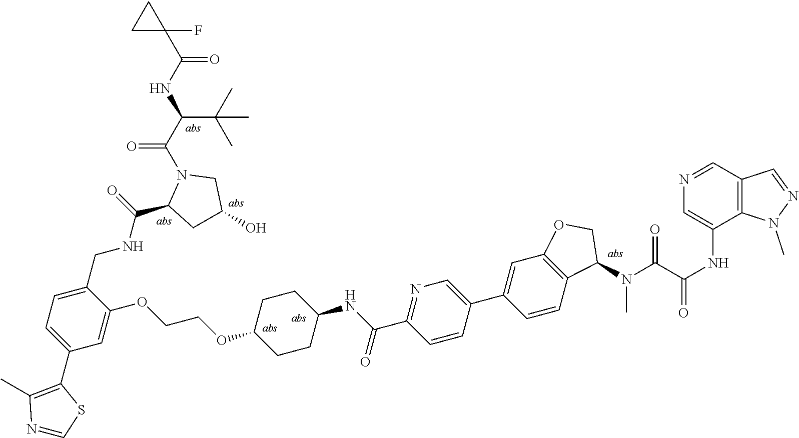 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)C(=O)Nc4cncc5cnn(C)c45)cn3)CC2)c1] |
| A326 | 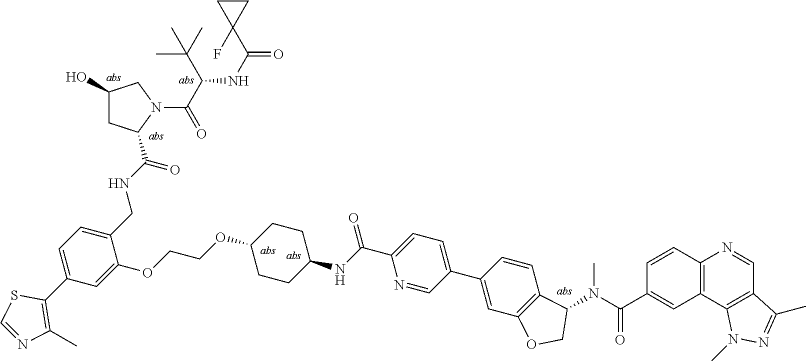 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5ncc6c(C)nn(C)c6c5c4)cn3)CC2)c1] |
| A327 | 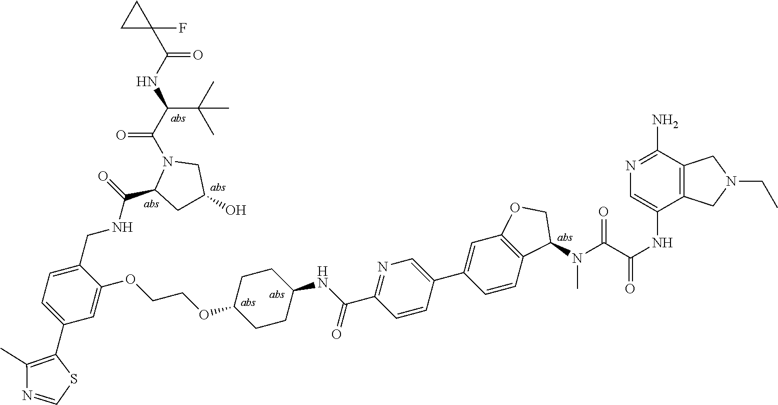 [SMILES: CCN1Cc2c(NC(=O)C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cnc(N)c2C1] |
| A328 | 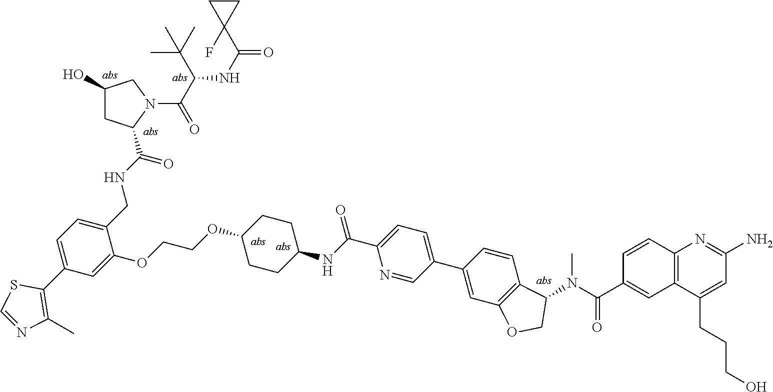 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)cc(CCCO)c5c4)cn3)CC2)c1] |
| A329 | 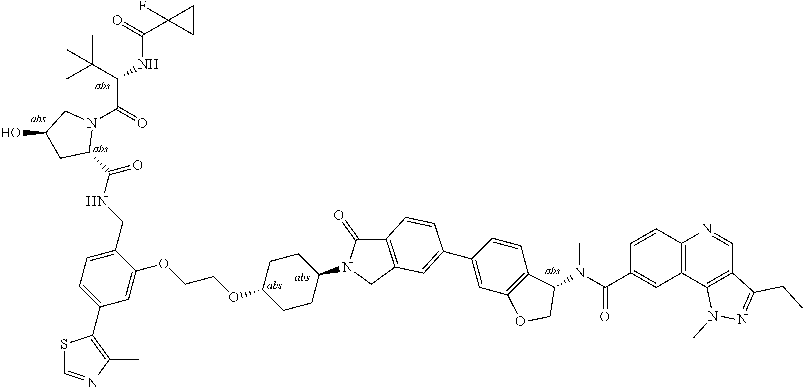 [SMILES: CCc1nn(C)c2c1cnc1ccc(C(=O)N(C)[C@@H]3COc4cc(-c5ccc6c(c5)CN([C@H]5CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC5)C6=O)ccc43)cc12] |
| A330 | 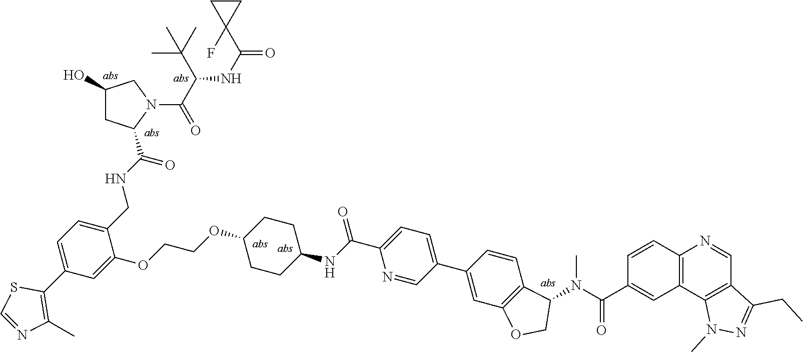 [SMILES: CCc1nn(C)c2c1cnc1ccc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc12] |
| A331 | 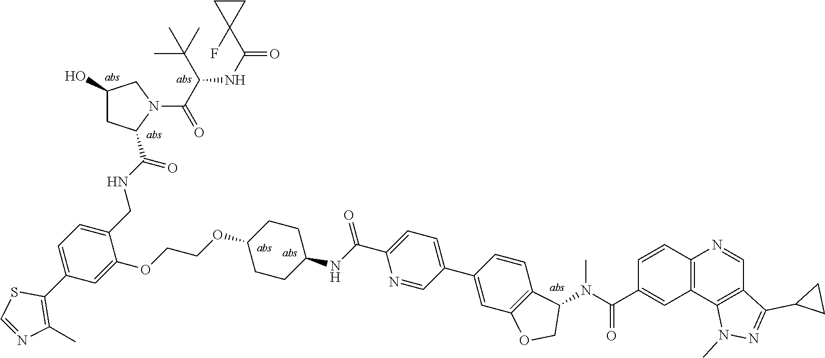 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5ncc6c(C7CC7)nn(C)c6c5c4)cn3)CC2)c1] |
| A334 | 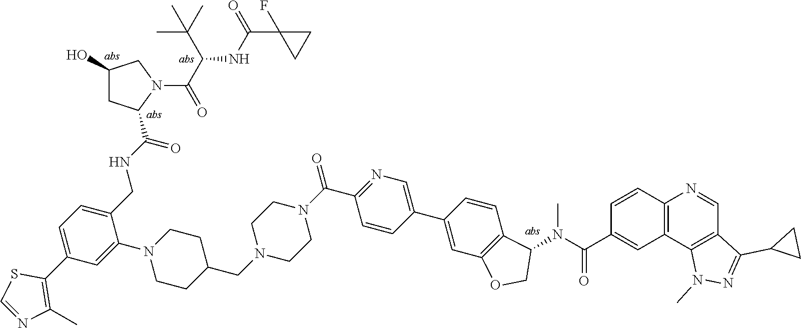 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncc7c(C8CC8)nn(C)c7c6c5)cn4)CC3)CC2)c1] |
| A335 | 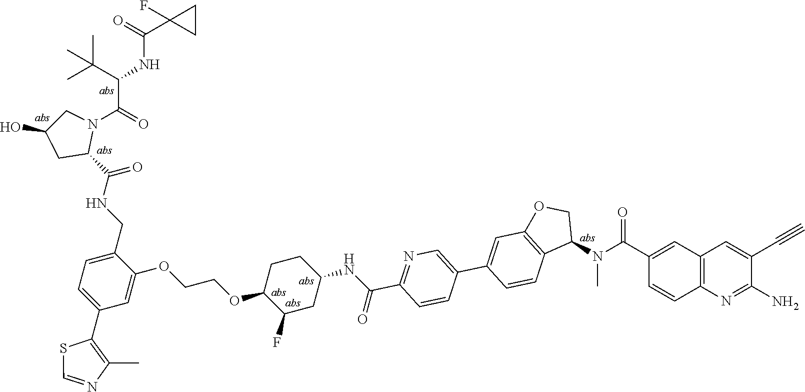 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)[C@H](F)C6)nc5)ccc43)ccc2nc1N] |
| A336 | 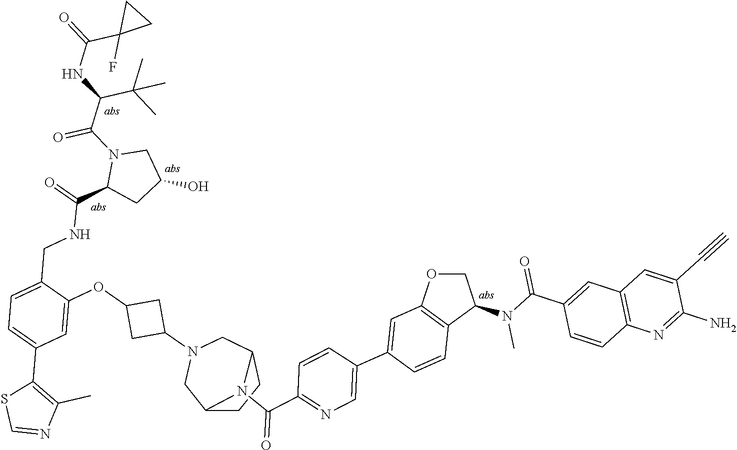 [SMILES: C#Cc1cc2cc(C(=O)N(C)[C@@H]3COc4cc(-c5ccc(C(=O)N6C7CCC6CN(C6CC(Oc8cc(-c9scnc9C)ccc8CNC(=O)[C@@H]8C[C@@H](O)CN8C(=O)[C@@H](NC(=O)C8(F)CC8)C(C)(C)C)C6)C7)nc5)ccc43)ccc2nc1N] |
| A337 | 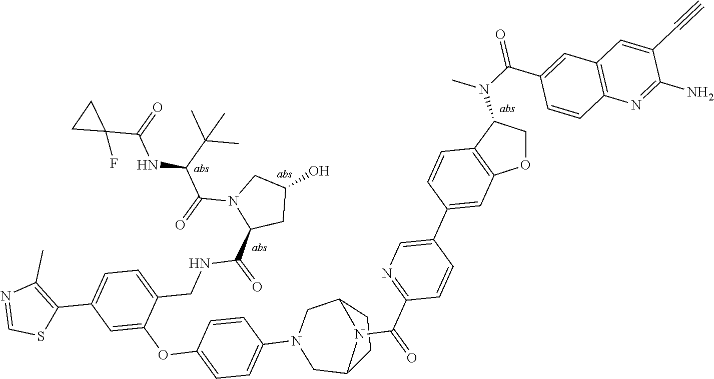 [SMILES: C#Cc1cc2cc(C(=O)N(C)[C@@H]3COc4cc(-c5ccc(C(=O)N6C7CCC6CN(c6ccc(Oc8cc(-c9scnc9C)ccc8CNC(=O)[C@@H]8C[C@@H](O)CN8C(=O)[C@@H](NC(=O)C8(F)CC8)C(C)(C)C)cc6)C7)nc5)ccc43)ccc2nc1N] |
| A338 | 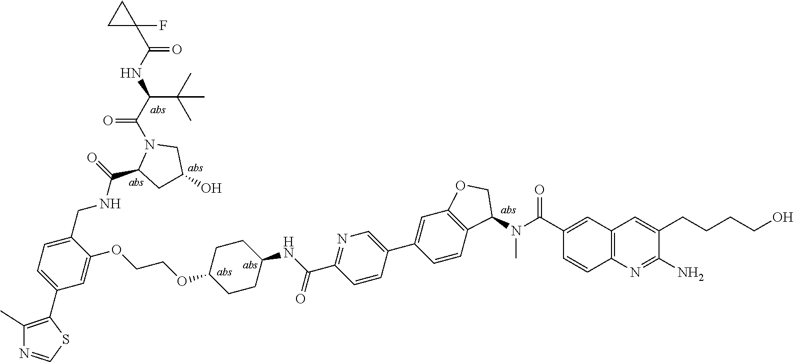 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(CCCCO)cc5c4)cn3)CC2)c1] |
| A339 | 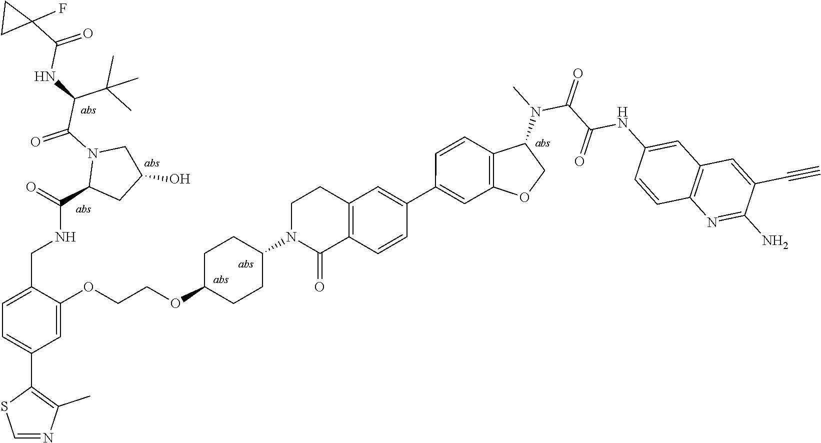 [SMILES: C#Cc1cc2cc(NC(=O)C(=O)N(C)[C@@H]3COc4cc(-c5ccc6c(c5)CCN([C@H]5CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC5)C6=O)ccc43)ccc2nc1N] |
| A341 | 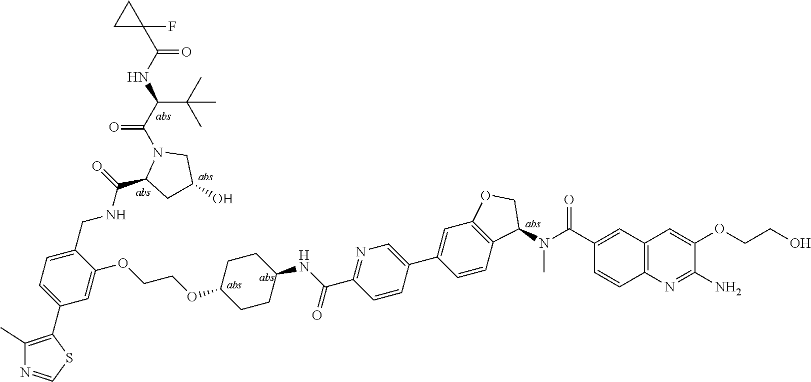 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(OCCO)cc5c4)cn3)CC2)c1] |
| A342 | 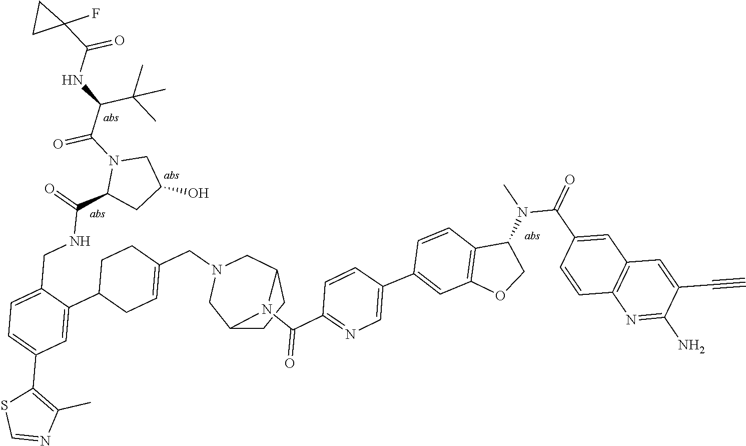 [SMILES: C#Cc1cc2cc(C(=O)N(C)[C@@H]3COc4cc(-c5ccc(C(=O)N6C7CCC6CN(CC6=CCC(c8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A343 | 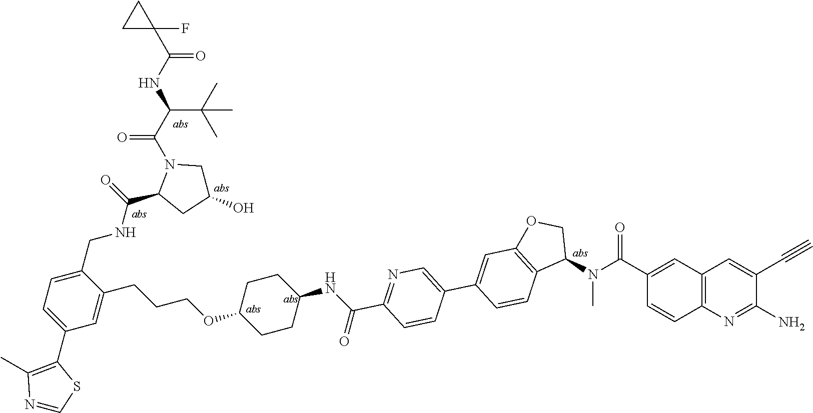 [SMILES: C#Cc1cc2cc(C(=O)N(C)[C@@H]3COc4cc(-c5ccc(C(=O)N[C@H]6CC[C@H](OCCCc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A344 | 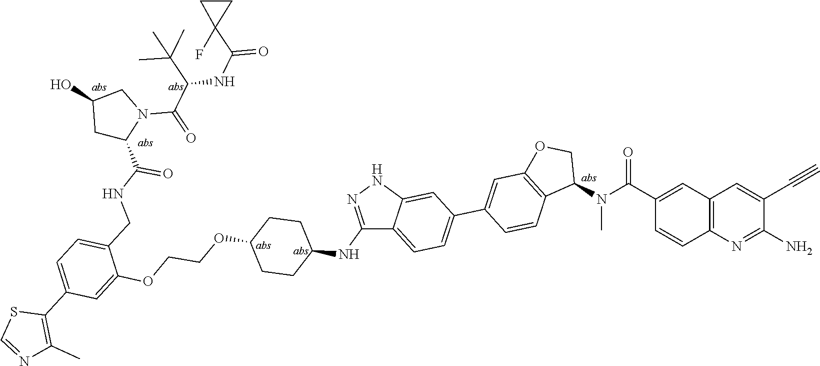 [SMILES: C#Cc1cc2cc(C(=O)N(C)[C@@H]3COc4cc(-c5ccc6c(N[C@H]7CC[C@H](OCCOc8cc(-c9scnc9C)ccc8CNC(=O)[C@@H]8C[C@@H](O)CN8C(=O)[C@@H](NC(=O)C8(F)CC8)C(C)(C)C)CC7)n[nH]c6c5)ccc43)ccc2nc1N] |
| A345 | 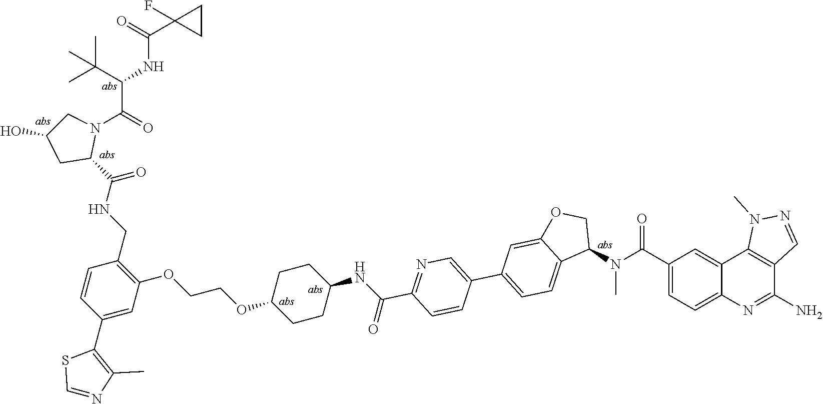 [SMILES: Cc1ncsc1-c1ccc(CNC(=O)[C@@H]2C[C@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6cnn(C)c6c5c4)cn3)CC2)c1] |
| A346 | 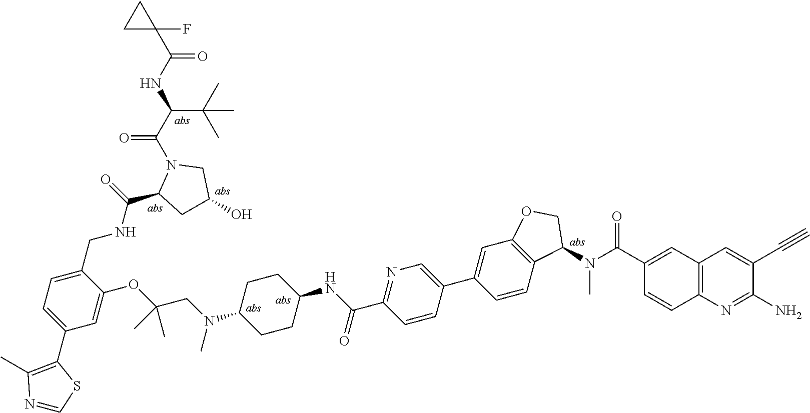 [SMILES: C#Cc1cc2cc(C(=O)N(C)[C@@H]3COc4cc(-c5ccc(C(=O)N[C@H]6CC[C@H](N(C)CC(C)(C)Oc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A347 | 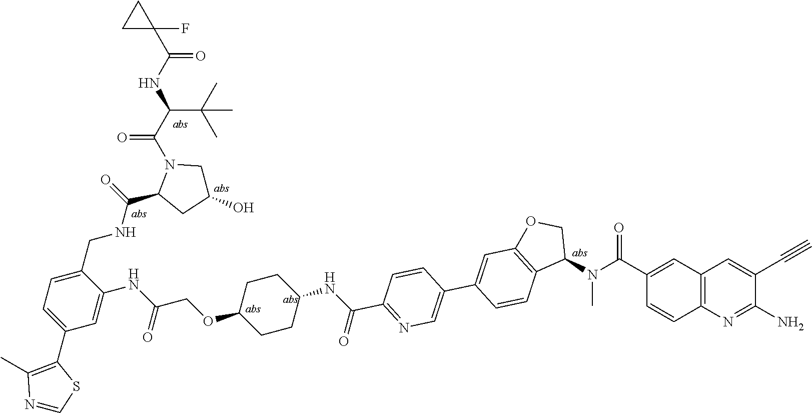 [SMILES: C#Cc1cc2cc(C(=O)N(C)[C@@H]3COc4cc(-c5ccc(C(=O)N[C@H]6CC[C@H](OCC(=O)Nc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A350 | 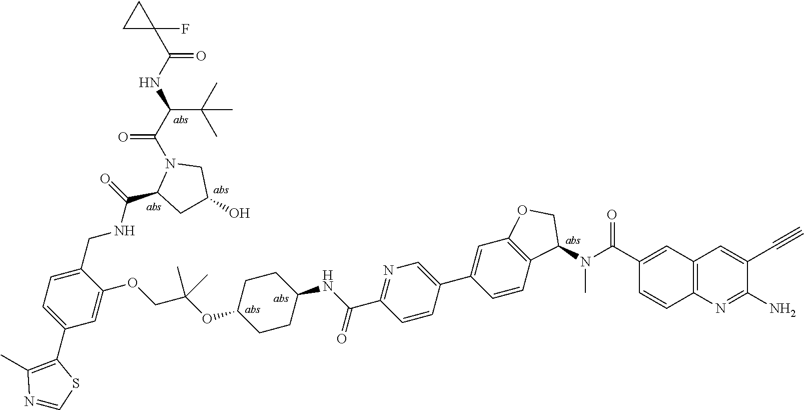 [SMILES: C#Cc1cc2cc(C(=O)N(C)[C@@H]3COc4cc(-c5ccc(C(=O)N[C@H]6CC[C@H](OC(C)(C)COc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A352 | 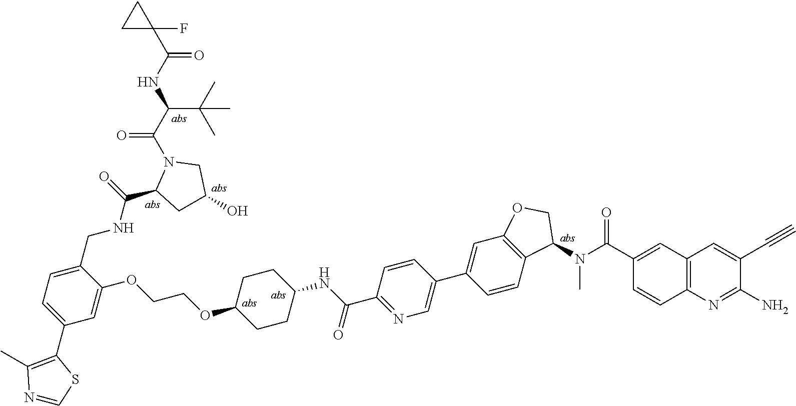 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A353 | 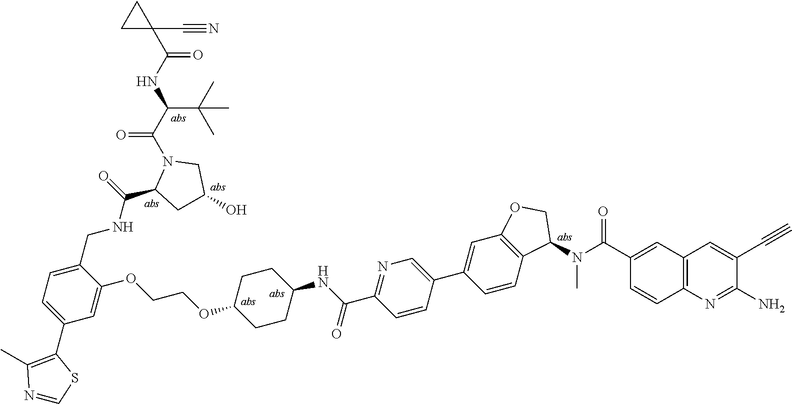 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(C#N)CC7)C(C)(C)C)CC6)nc5)ccc43)ccc2nc1N] |
| A354 | 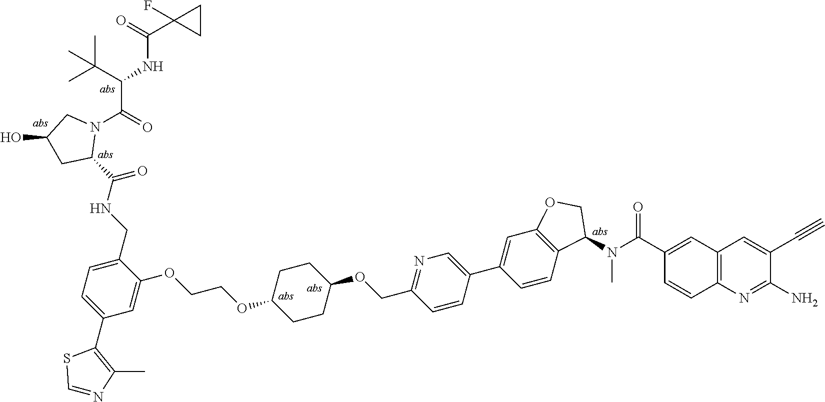 [SMILES: C#Cc1cc2cc(C(=O)N(C)[C@@H]3COc4cc(-c5ccc(CO[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A355 | 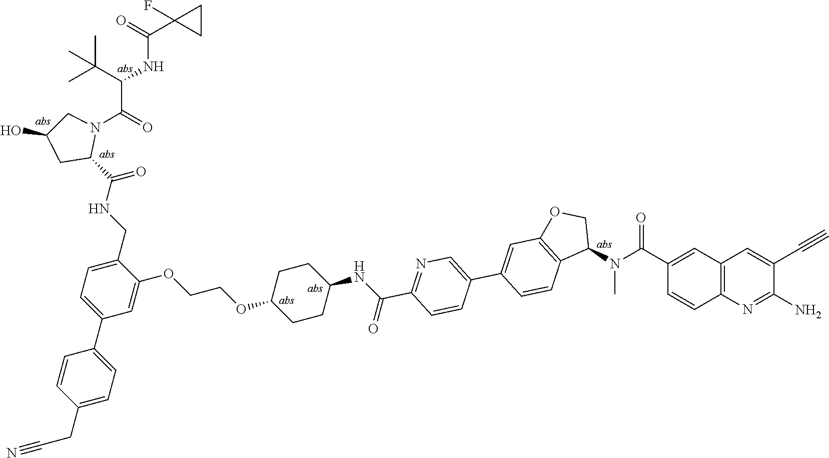 [SMILES: C#Cc1cc2cc(C(=O)N(C)[C@@H]3COc4cc(-c5ccc(C(=O)N[C@H]6CC[C@H](OCCOc7cc(-c8ccc(CC#N)cc8)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A357 | 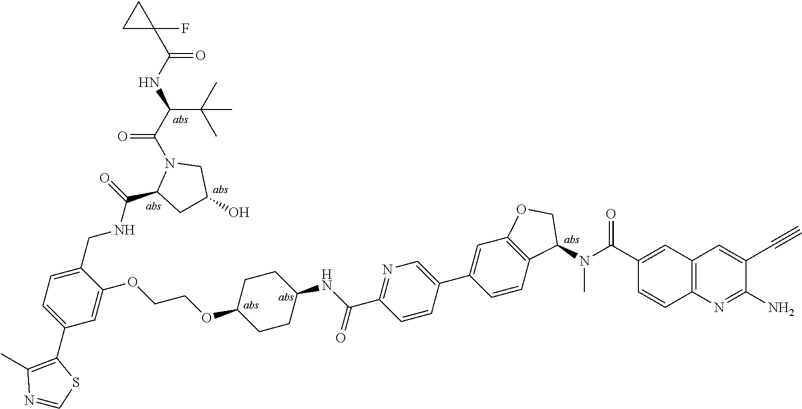 [SMILES: C#Cc1cc2cc(C(=O)N(C)[C@@H]3COc4cc(-c5ccc(C(=O)N[C@H]6CC[C@@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A358 | 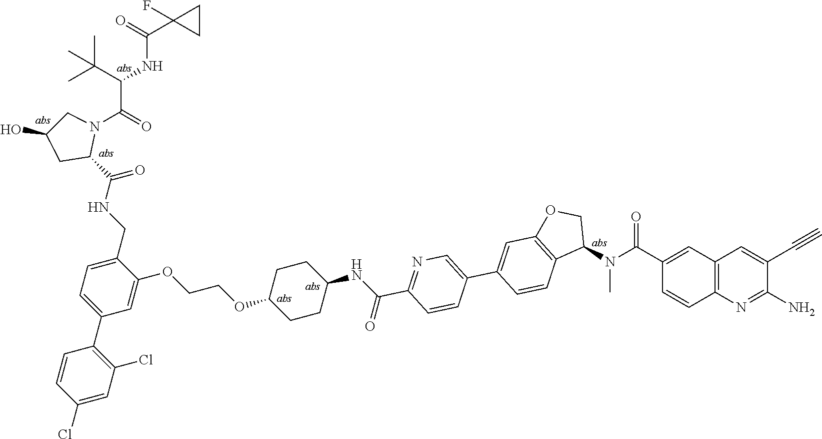 [SMILES: C#Cc1cc2cc(C(=O)N(C)[C@@H]3COc4cc(-c5ccc(C(=O)N[C@H]6CC[C@H](OCCOc7cc(-c8ccc(Cl)cc8Cl)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A359 | 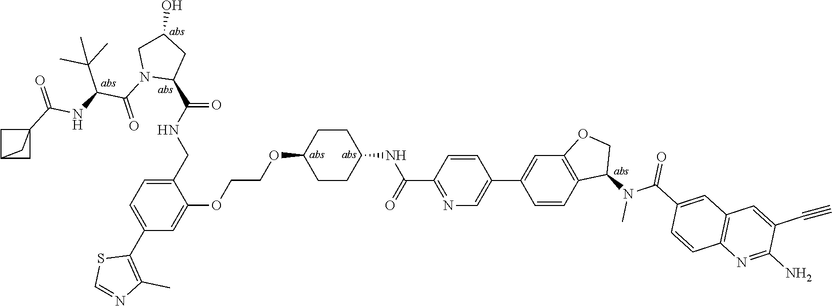 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C78CC(C7)C8)C(C)(C)C)CC6)nc5)ccc43)ccc2nc1N] |
| A360 | 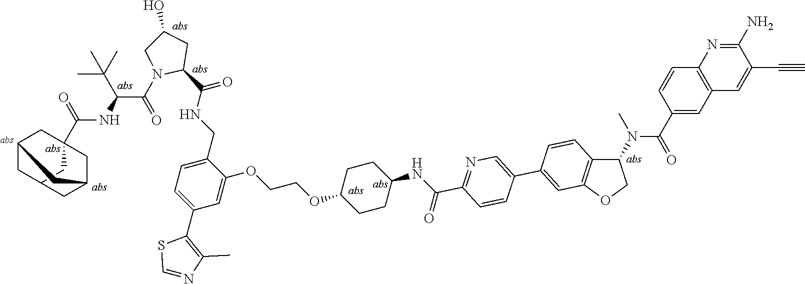 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)[C@]78CC9C[C@H](C[C@H](C9)C7)C8)C(C)(C)C)CC6)nc5)ccc43)ccc2nc1N] |
| A363 | 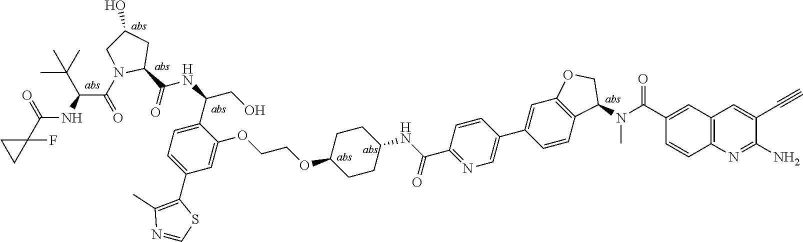 [SMILES: C#Cc1cc2cc(C(=O)N(C)[C@@H]3COc4cc(-c5ccc(C(=O)N[C@H]6CC[C@H](OCCOc7cc(-c8scnc8C)ccc7[C@H](CO)NC(=O)[C@@H]7C[C@@H](O)CN7C(=O)[C@@H](NC(=O)C7(F)CC7)C(C)(C)C)CC6)nc5)ccc43)ccc2nc1N] |
| A364 | 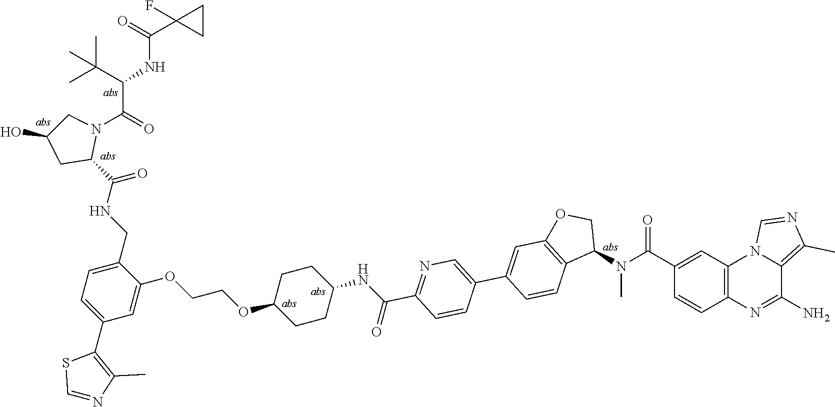 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6c(C)ncn6c5c4)cn3)CC2)c1] |
| A365 | 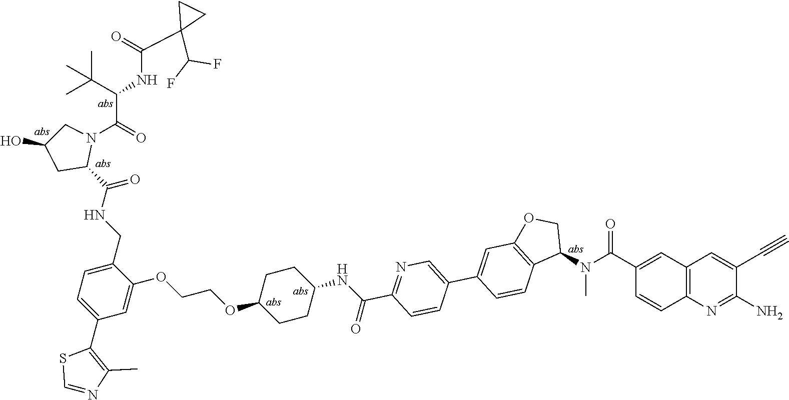 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(C(F)F)CC7)C(C)(C)C)CC6)nc5)ccc43)ccc2nc1N] |
| A366 | 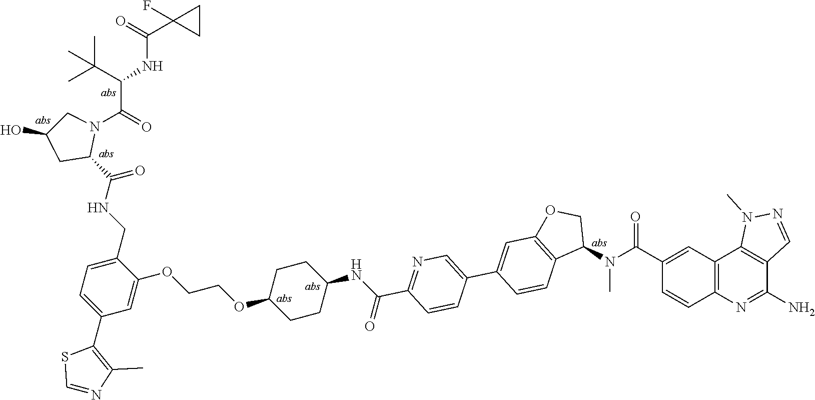 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@@H](NC(=O)c3ccc(-c4ccc5c(c4)OC[C@H]5N(C)C(=O)c4ccc5nc(N)c6cnn(C)c6c5c4)cn3)CC2)c1] |
| A367 | 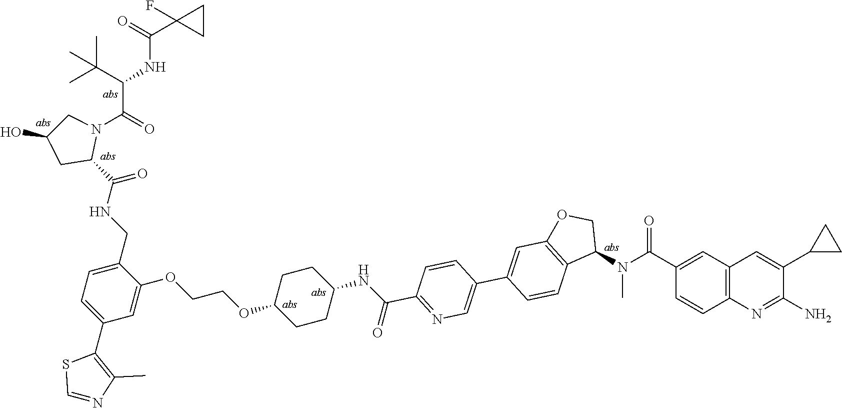 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@@H](NC(=O)c3ccc(-c4ccc5c(c4)OC[C@H]5N(C)C(=O)c4ccc5nc(N)c(C6CC6)cc5c4)cn3)CC2)c1] |
| A368 | 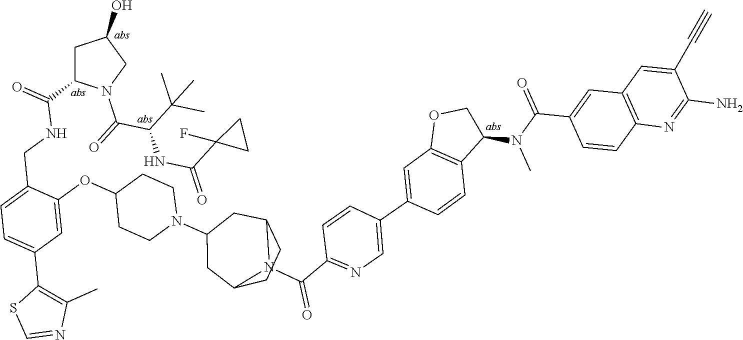 [SMILES: C#Cc1cc2cc(C(=O)N(C)[C@@H]3COc4cc(-c5ccc(C(=O)N6C7CCC6CC(N6CCC(Oc8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N] |
| A369 | 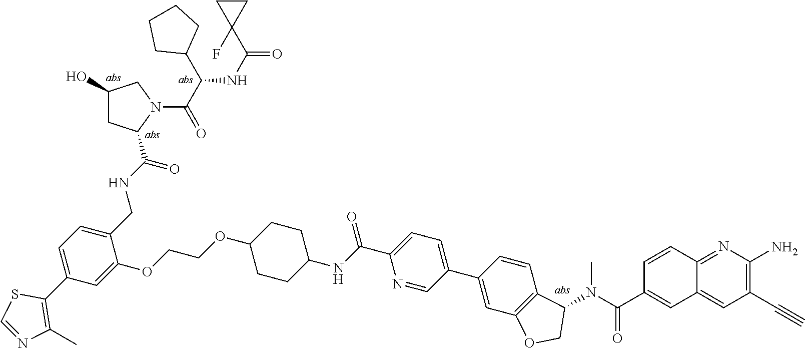 [SMILES: C#Cc1cc2cc(C(=O)N(C)[C@@H]3COc4cc(-c5ccc(C(=O)NC6CCC(OCCOc7cc(-c8scnc8C)ccc7CNC(=O)[C@@H]7C[C@@H](O)CN7C(=O)[C@@H](NC(=O)C7(F)CC7)C7CCCC7)CC6)nc5)ccc43)ccc2nc1N] |
| A370 | 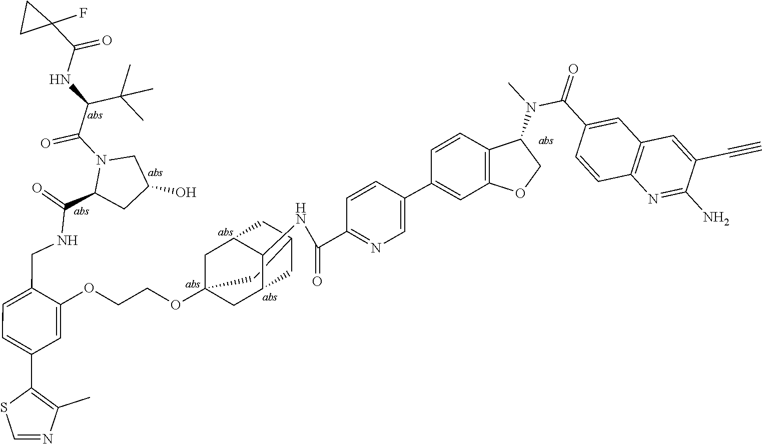 [SMILES: C#Cc1cc2cc(C(=O)N(C)[C@@H]3COc4cc(-c5ccc(C(=O)NC6[C@@H]7CC8C[C@H]6C[C@@](OCCOc6cc(-c9scnc9C)ccc6CNC(=O)[C@@H]6C[C@@H](O)CN6C(=O)[C@@H](NC(=O)C6(F)CC6)C(C)(C)C)(C8)C7)nc5)ccc43)ccc2nc1N] |
| A371 | 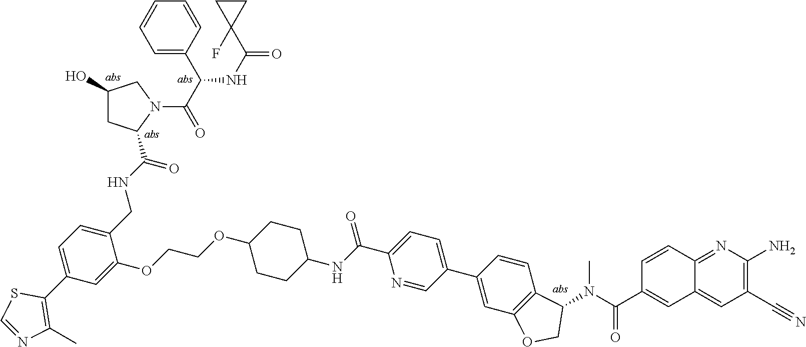 [SMILES: Cc1ncsc1-c1ccc(CNC(=O)[C@@H]2C[C@@H](O)CN2C(=O)[C@@H](NC(=O)C2(F)CC2)c2ccccc2)c(OCCOC2CCC(NC(=O)c3ccc(-c4ccc5c(c4)OC[C@H]5N(C)C(=O)c4ccc5nc(N)c(C#N)cc5c4)cn3)CC2)c1] |
| A373 | 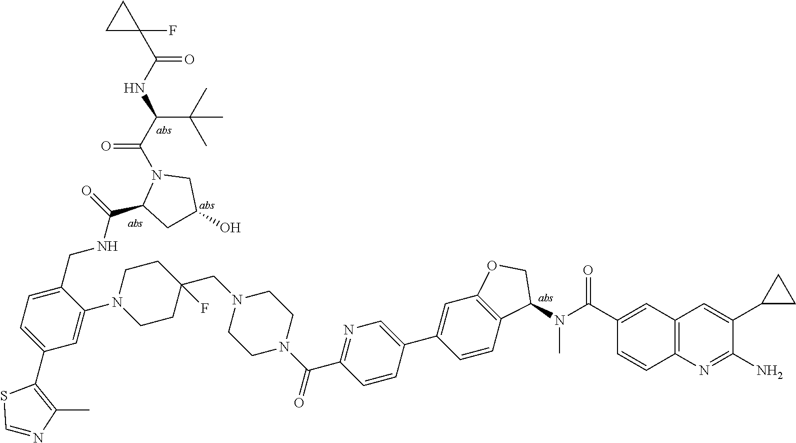 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(F)(CN3CCN(C(=O)c4ccc(-c5ccc6c(c5)OC[C@H]6N(C)C(=O)c5ccc6nc(N)c(C7CC7)cc6c5)cn4)CC3)CC2)c1] |
| A374 | 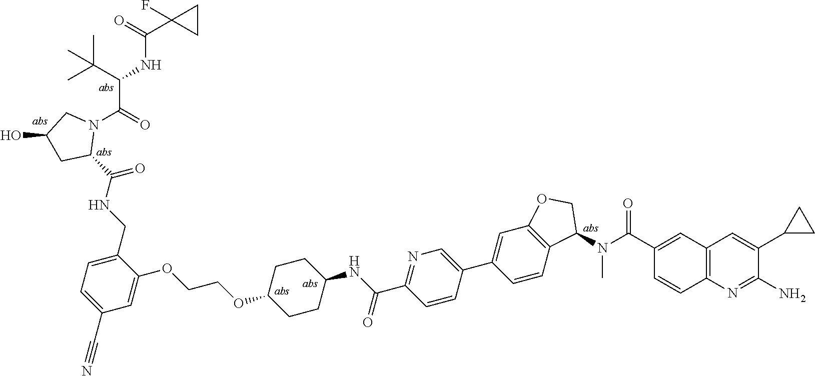 [SMILES: CN(C(=O)c1ccc2nc(N)c(C3CC3)cc2c1)[C@@H]1COc2cc(-c3ccc(C(=O)N[C@H]4CC[C@H](OCCOc5cc(C#N)ccc5CNC(=O)[C@@H]5C[C@@H](O)CN5C(=O)[C@@H](NC(=O)C5(F)CC5)C(C)(C)C)CC4)nc3)ccc21] |
| A375 | 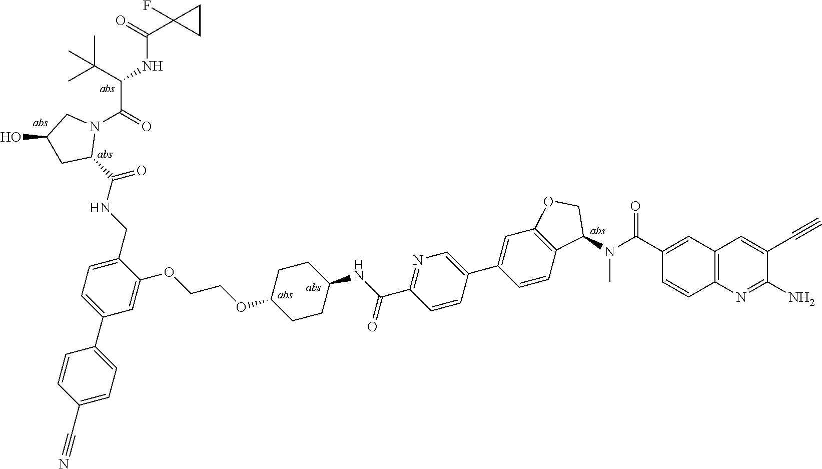 [SMILES: C#Cc1cc2cc(C(=O)N(C)[C@@H]3COc4cc(-c5ccc(C(=O)N[C@H]6CC[C@H](OCCOc7cc(-c8ccc(C#N)cc8)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A376 | 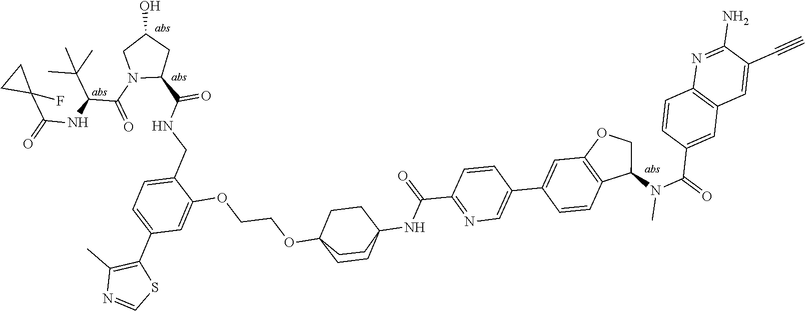 [SMILES: C#Cc1cc2cc(C(=O)N(C)[C@@H]3COc4cc(-c5ccc(C(=O)NC67CCC(OCCOc8cc(-c9scnc9C)ccc8CNC(=O)[C@@H]8C[C@@H](O)CN8C(=O)[C@@H](NC(=O)C8(F)CC8)C(C)(C)C)(CC6)CC7)nc5)ccc43)ccc2nc1N] |
| A378 | 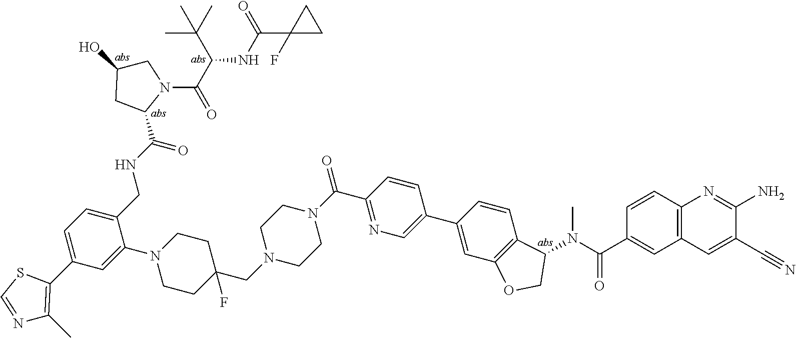 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(F)(CN3CCN(C(=O)c4ccc(-c5ccc6c(c5)OC[C@H]6N(C)C(=O)c5ccc6nc(N)c(C#N)cc6c5)cn4)CC3)CC2)c1] |
| A379 | 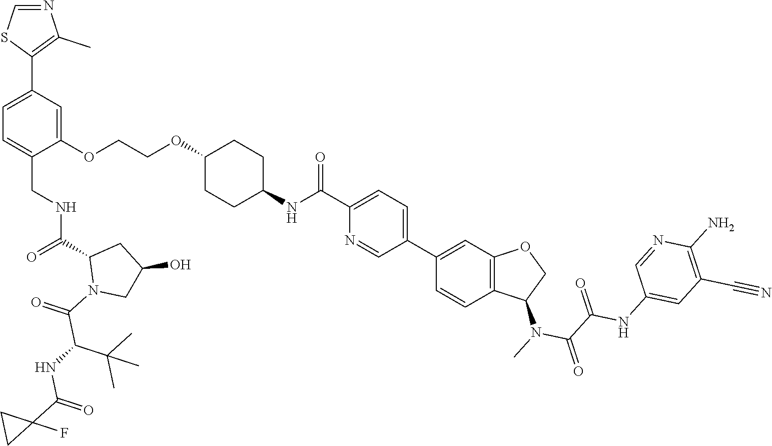 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)C(=O)Nc4cnc(N)c(C#N)c4)cn3)CC2)c1] |
| A380 | 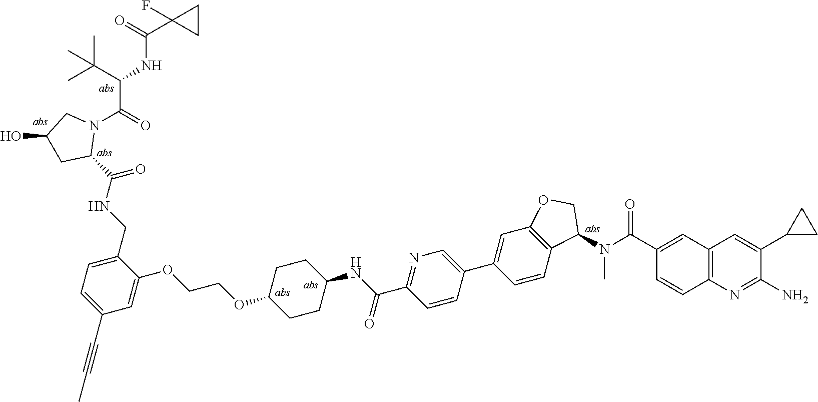 [SMILES: CC#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CC6)cc5c4)cn3)CC2)c1] |
| A383 | 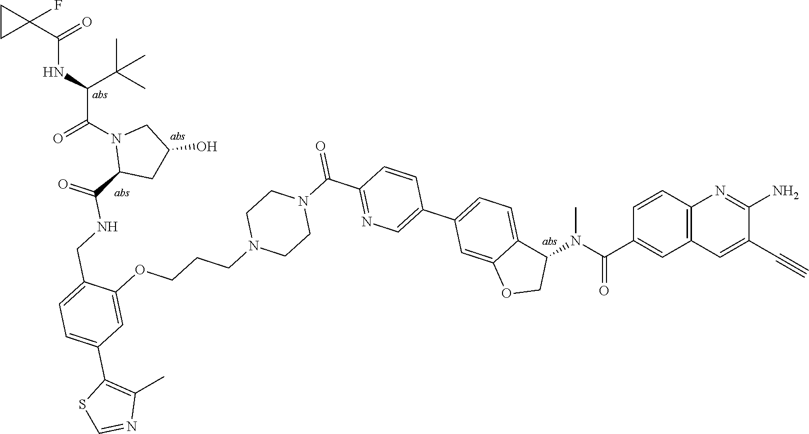 [SMILES: C#Cc1cc2cc(C(=O)N(C)[C@@H]3COc4cc(-c5ccc(C(=O)N6CCN(CCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A384 | 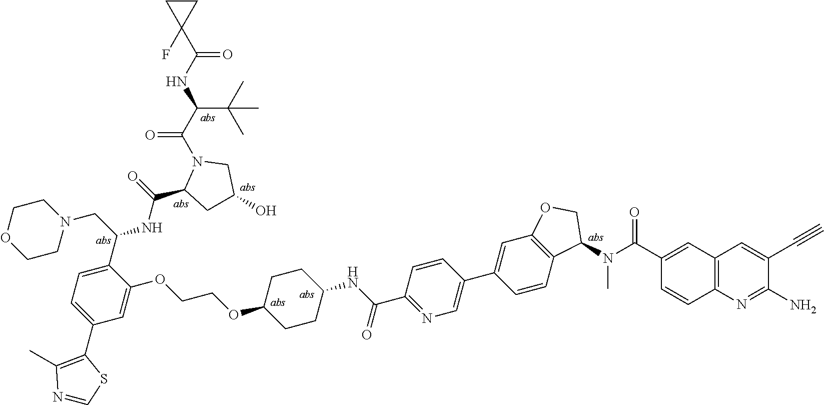 [SMILES: C#Cc1cc2cc(C(=O)N(C)[C@@H]3COc4cc(-c5ccc(C(=O)N[C@H]6CC[C@H](OCCOc7cc(-c8scnc8C)ccc7[C@H](CN7CCOCC7)NC(=O)[C@@H]7C[C@@H](O)CN7C(=O)[C@@H](NC(=O)C7(F)CC7)C(C)(C)C)CC6)nc5)ccc43)ccc2nc1N] |
| A385 | 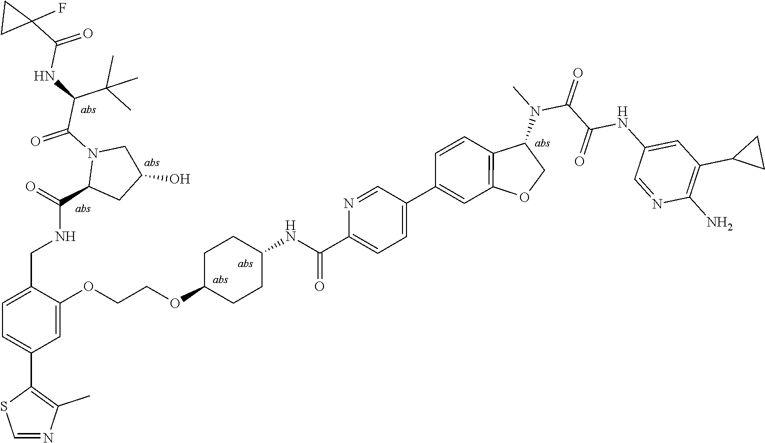 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)C(=O)Nc4cnc(N)c(C5CC5)c4)cn3)CC2)c1] |
| A386 | 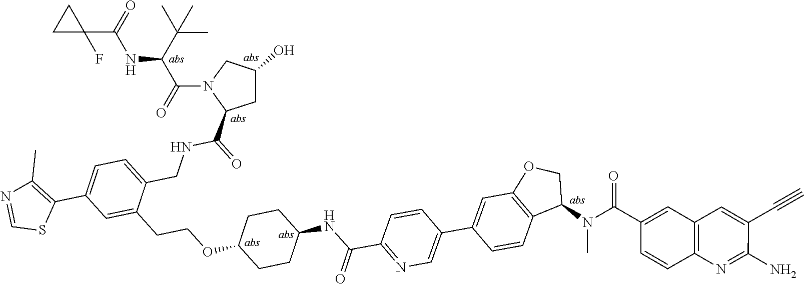 [SMILES: C#Cc1cc2cc(C(=O)N(C)[C@@H]3COc4cc(-c5ccc(C(=O)N[C@H]6CC[C@H](OCCc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N] |
| A387 | 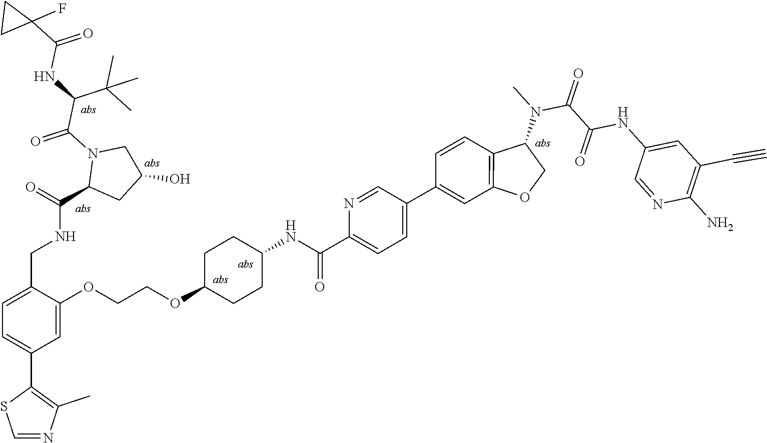 [SMILES: C#Cc1cc(NC(=O)C(=O)N(C)[C@@H]2COc3cc(-c4ccc(C(=O)N[C@H]5CC[C@H](OCCOc6cc(-c7scnc7C)ccc6CNC(=O)[C@@H]6C[C@@H](O)CN6C(=O)[C@@H](NC(=O)C6(F)CC6)C(C)(C)C)CC5)nc4)ccc32)cnc1N] |
| A389 | 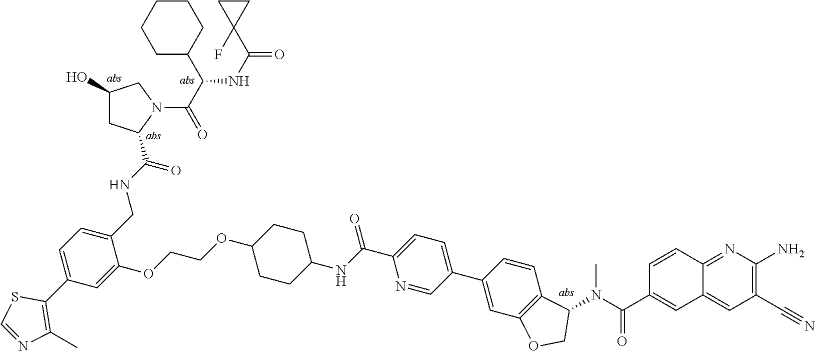 [SMILES: Cc1ncsc1-c1ccc(CNC(=O)[C@@H]2C[C@@H](O)CN2C(=O)[C@@H](NC(=O)C2(F)CC2)C2CCCCC2)c(OCCOC2CCC(NC(=O)c3ccc(-c4ccc5c(c4)OC[C@H]5N(C)C(=O)c4ccc5nc(N)c(C#N)cc5c4)cn3)CC2)c1] |
| A390 | 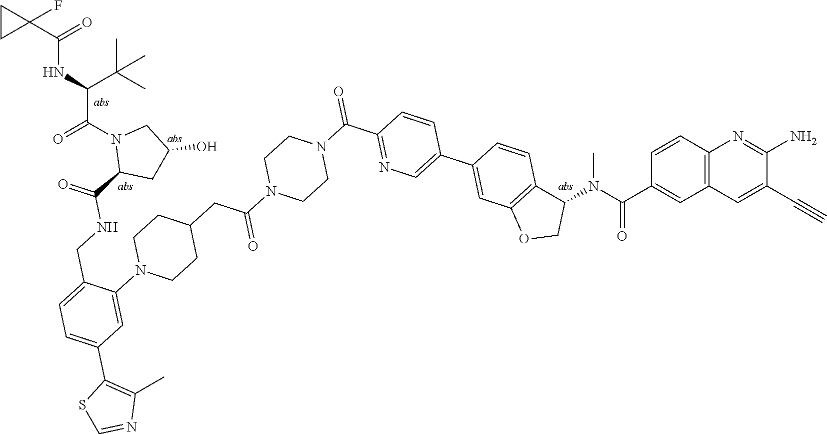 [SMILES: C#Cc1cc2cc(C(=O)N(C)[C@@H]3COc4cc(-c5ccc(C(=O)N6CCN(C(=O)CC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N] |
| A391 | 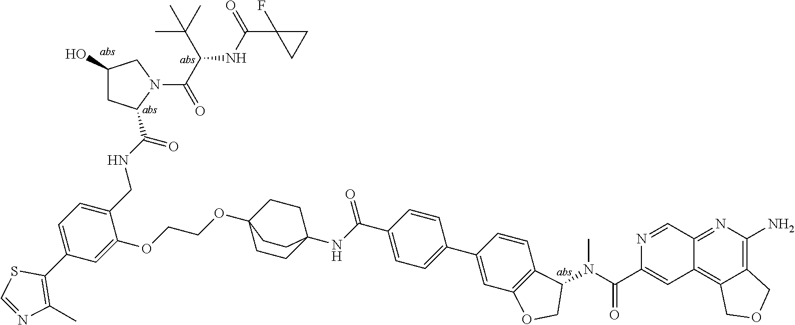 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOC23CCC(NC(=O)c4ccc(-c5ccc6c(c5)OC[C@H]6N(C)C(=O)c5cc6c7c(c(N)nc6cn5)COC7)cc4)(CC2)CC3)c1] |
| A392 | 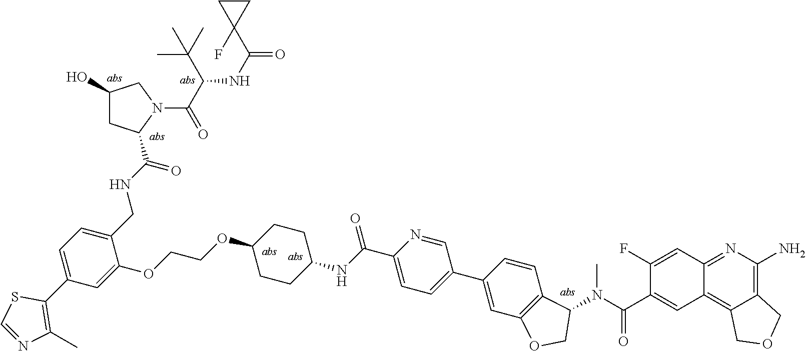 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4cc5c6c(c(N)nc5cc4F)COC6)cn3)CC2)c1] |
| A393 | 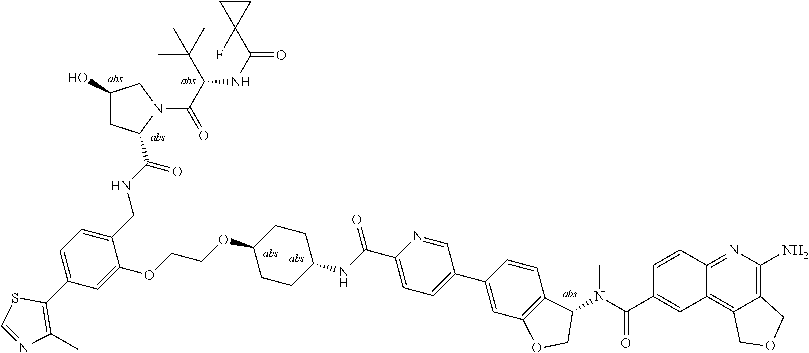 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6c(c5c4)COC6)cn3)CC2)c1] |
| A394 | 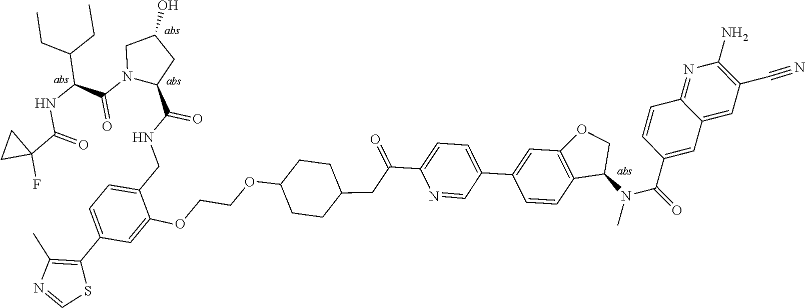 [SMILES: CCC(CC)[C@H](NC(=O)C1(F)CC1)C(=O)N1C[C@H](O)C[C@H]1C(=O)NCc1ccc(-c2scnc2C)cc1OCCOC1CCC(CC(=O)c2ccc(-c3ccc4c(c3)OC[C@H]4N(C)C(=O)c3ccc4nc(N)c(C#N)cc4c3)cn2)CC1] |
| A395 | 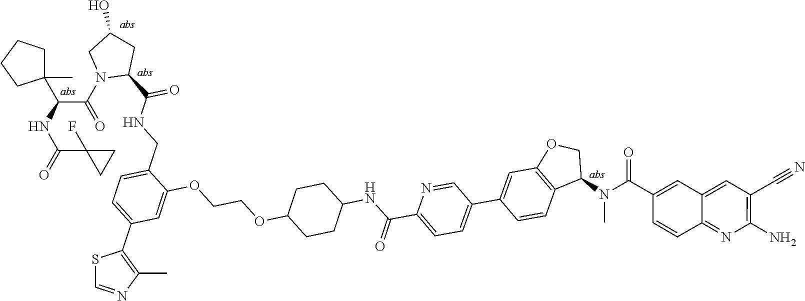 [SMILES: Cc1ncsc1-c1ccc(CNC(=O)[C@@H]2C[C@@H](O)CN2C(=O)[C@@H](NC(=O)C2(F)CC2)C2(C)CCCC2)c(OCCOC2CCC(NC(=O)c3ccc(-c4ccc5c(c4)OC[C@H]5N(C)C(=O)c4ccc5nc(N)c(C#N)cc5c4)cn3)CC2)c1] |
| A396 | 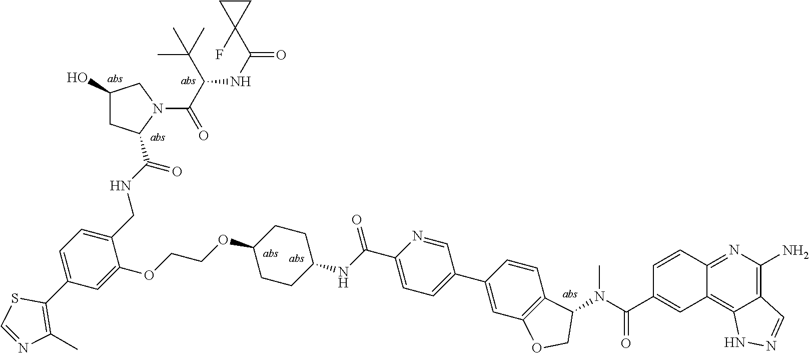 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6cn[nH]c6c5c4)cn3)CC2)c1] |
| A397 | 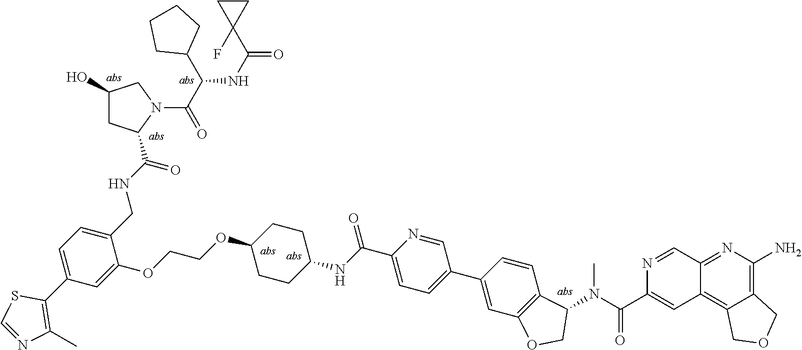 [SMILES: Cc1ncsc1-c1ccc(CNC(=O)[C@@H]2C[C@@H](O)CN2C(=O)[C@@H](NC(=O)C2(F)CC2)C2CCCC2)c(OCCO[C@H]2CC[C@H](NC(=O)c3ccc(-c4ccc5c(c4)OC[C@H]5N(C)C(=O)c4cc5c6c(c(N)nc5cn4)COC6)cn3)CC2)c1] |
| A398 | 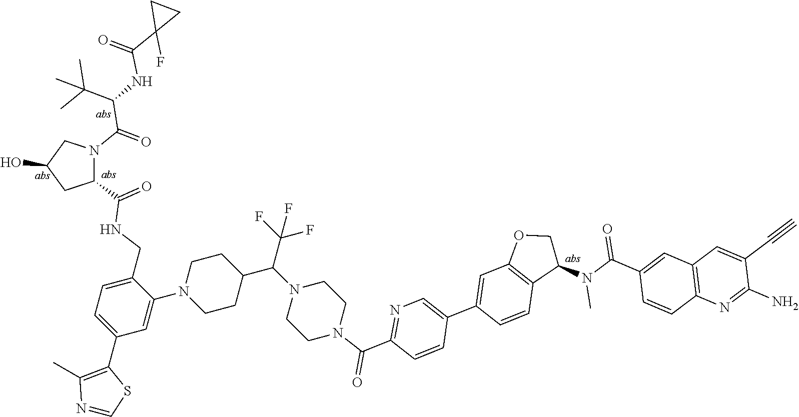 [SMILES: C#Cc1cc2cc(C(=O)N(C)[C@@H]3COc4cc(-c5ccc(C(=O)N6CCN(C(C7CCN(c8cc(-c9scnc9C)ccc8CNC(=O)[C@@H]8C[C@@H](O)CN8C(=O)[C@@H](NC(=O)C8(F)CC8)C(C)(C)C)CC7)C(F)(F)F)CC6)nc5)ccc43)ccc2nc1N] |
| A399 | 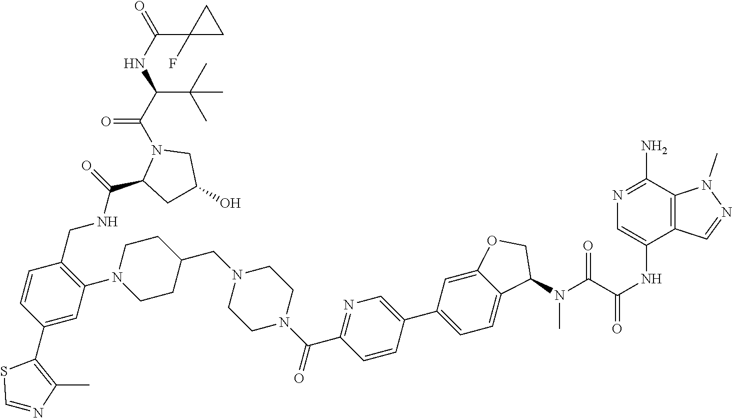 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)C(=O)Nc5cnc(N)c6c5cnn6C)cn4)CC3)CC2)c1] |
| A400 | 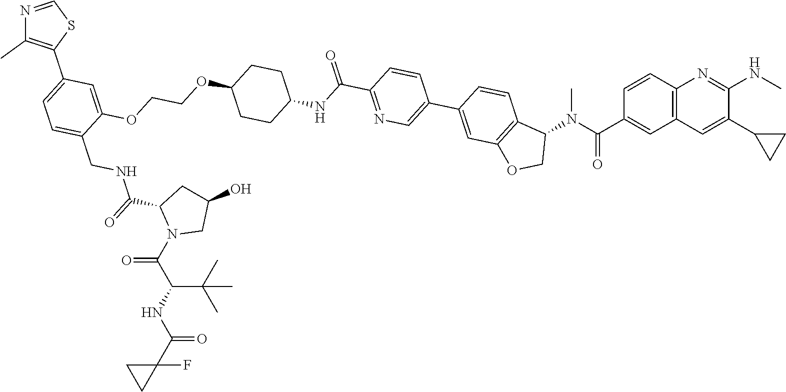 [SMILES: CNc1nc2ccc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2cc1C1CC1] |
| A401 | 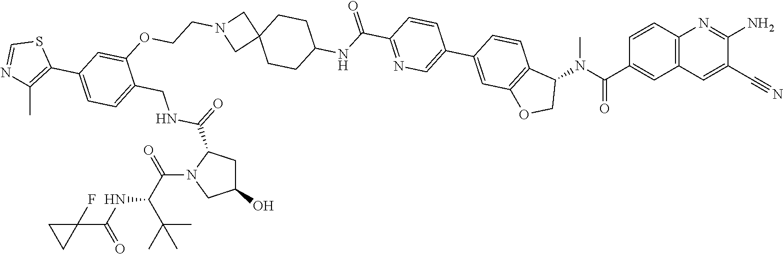 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CC3(CCC(NC(=O)c4ccc(-c5ccc6c(c5)OC[C@H]6N(C)C(=O)c5ccc6nc(N)c(C#N)cc6c5)cn4)CC3)C2)c1] |
| A402 | 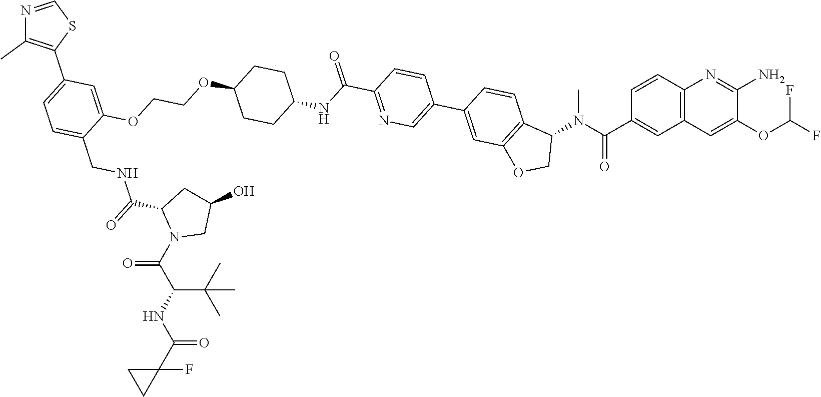 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(OC(F)F)cc5c4)cn3)CC2)c1] |
| A403 | 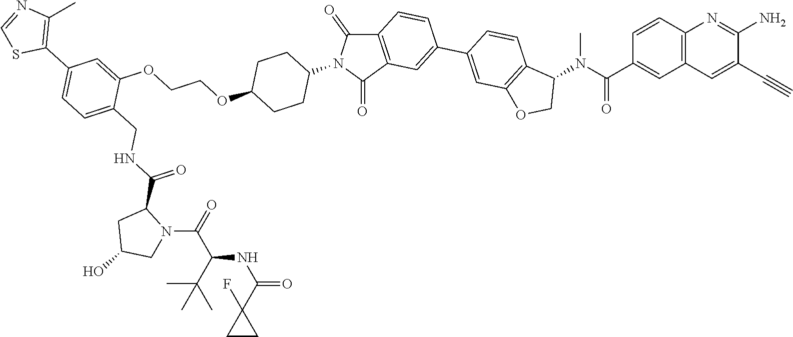 |

| Compound No. | |
|---|---|
| A1 | 4-amino-N-((S)-6-(6-((17-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)-3,6,9,12,15-pentaoxaheptadecyl) |
| | carbamoyl)pyridin-3-yl)-2,3-dihydrobenzofuran-3-yl)-N,1-dimethyl-1H-pyrazolo[4,3-c]quinoline-8-carboxamide |
| A2 | 4-amino-N-((S)-6-(6-((14-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)-3,6,9,12-tetraoxatetradecyl)carbamoyl)- |
| | pyridin-3-yl)-2,3-dihydrobenzofuran-3-yl)-N,1-dimethyl-1H-pyrazolo[4,3-c]quinoline-8-carboxamide |
| A3 | 4-amino-N-((S)-6-(6-((20-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)-3,6,9,12,15,18-hexaoxaicosyl) |
| | carbamoyl)pyridin-3-yl)-2,3-dihydrobenzofuran-3-yl)-N,1-dimethyl-1H-pyrazolo[4,3-c]quinoline-8-carboxamide |
| A4 | 4-amino-N-((S)-6-(6-((2-(2-(2-(2-(2-(((2S,4R)-1-((S)-2-(1-fluorocyclopropane-1-carboxamido)-3,3-dimethyl- |
| | butanoyl)-4-hydroxypyrrolidine-2-carboxamido)methyl)-5-(4-methylthiazol-5-yl)phenoxy)ethoxy)ethoxy)ethyl) |
| | carbamoyl)pyridin-3-yl)-2,3-dihydrobenzofuran-3-yl)-N,1-dimethyl-1H-pyrazolo[4,3-c]quinoline-8-carboxamide |
| A5 | 4-amino-N-((S)-6-(6-((11-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)undecyl)carbamoyl)pyridin-3-yl)- |
| | 2,3-dihydrobenzofuran-3-yl)-N,1-dimethyl-1H-pyrazolo[4,3-c]quinoline-8-carboxamide |
| A6 | 4-amino-N-((S)-6-(6-((2-(2-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)ethoxy)ethyl) |
| | carbamoyl)pyridin-3-yl)-2,3-dihydrobenzofuran-3-yl)-N,1-dimethyl-1H-pyrazolo[4,3-c]quinoline-8-carboxamide |
| A7 | 4-amino-N-((S)-6-(6-((2-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethoxy)ethyl)carbamoyl)pyrrolidin- |
| | 3-yl)-2,3-dihydrobenzofuran-3-yl)-N,1-dimethyl-1H-pyrazolo[4,3-c]quinoline-8-carboxamide |
| A8 | 4-amino-N-((S)-6-(6-((2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethyl)carbamoyl)pyridin-3-yl)-2,3- |
| | dihydrobenzofuran-3-yl)-N,1-dimethyl-1H-pyrazolo[4,3-c]quinoline-8-carboxamide |
| A9 | 4-amino-N-((S)-6-(6-((17-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)-3,6,9,12,15-pentaoxaheptadecyl) |
| | carbamoyl)pyridin-3-yl)-N-methyl-1,3-dihydrofuro[3,4-c][1,7]naphthyridine-8-carboxamide |
| A10 | 4-amino-N-((S)-6-(6-((14-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)-3,6,9,12-tetraoxatetradecyl)carbamoyl) |
| | pyridin-3-yl)-2,3-dihydrobenzofuran-3-yl)-N-methyl-1,3-dihydrofuro[3,4-c][1,7]naphthyridine-8-carboxamie |
| A11 | 4-amino-7-fluoro-N-((S)-6-(6-((17-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)-3,6,9,12,15- |
| | pentaoxaheptadecyl)carbamoyl)pyridin-3-yl)-2,3-dihydrobenzofuran-3-yl)-N-methyl-1,3-dihydrofuro[3,4-c]quinoline-8- |
| | carboxamide |
| A12 | 4-amino-7-fluoro-N-((S)-6-(6-((14-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)-3,6,9,12-tetraoxatetradecyl)carbamoyl) |
| | pyridin-3-yl)-2,3-dihydrobenzofuran-3-yl)-N-methyl-1,3-dihydrofuro[3,4-c]quinoline-8-carboxamide |
| A13 | 2-amino-N-((S)-6-(6-((14-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)-3,6,9,12-yl)-2,3-dihydrobenzofuran- |
| | 3-yl)-N,3-dimethylquinoline-6-carboxamide |
| A14 | 2-amino-N-((S)-6-(6-((17-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)-3,6,9,12,15-pentaoxaheptadecyl) |
| | carbamoyl)pyridin-3-yl)-2,3-dihydrobenzofuran-3-yl)-N,3-dimethylquinoline-6-carboxamide |
| A15 | 4-amino-N-((S)-6-(6-((9-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)nonyl)carbamoyl)pyridin-3-yl)-2,3- |
| | dihydrobenzofuran-3-yl)-N,1-dimethyl-1H-pyrazolo[4,3-c]quinoline-8-carboxamide |
| A16 | 4-amino-N-((S)-6-(6-((10-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)decyl)carbamoyl)pyridin-3-yl)- |
| | 2,3-dihydrobenzofuran-3-yl)-N,1-dimethyl-1H-pyrazolo[4,3-c]quinoline-8-carboxamide |
| A17 | 4-amino-N-((S)-6-(6-(4-(2-(2-(2-(2-(2-(((2S,4R)-1-((S)-2-(1-fluorocyclopropane-1-carboxamido)-3,3-dimethyl- |
| | butanoyl)-4-hydroxypyrrolidine-2-carboxamido)methyl)-5-(4-methylthiazol-5-yl)phenoxy)ethoxy)ethoxy)ethoxy) |
| | ethyl)piperazine-1-carbonyl)pyridin-3-yl)-2,3-dihydrobenzofuran-3-yl)-N,1-dimethyl-1H-pyrazolo[4,3-c] |
| | quinoline-8-carboxamide |
| A18 | 4-amino-N-((S)-6-(6-(9-(2-(2-(2-(2-(((2S,4R)-1-((S)-2-(1-fluorocyclopropane-1-carboxamido)-3,3-dimethyl- |
| | butanoyl)-4-hydroxypyrrolidine-2-carboxamido)methyl)-5-(4-methylthiazol-5-yl)phenoxy)ethoxy)ethoxy)ethyl)-3,9- |
| | diazaspiro[5.5]undecane-3-carbonyl)pyridin-3-yl)-2,3-dihydrobenzofuran-3-yl)-N,1-dimethyl- |
| | 1H-pyrazolo[4,3-c]quinoline-8-carboxamide |
| A19 | 4-amino-N-((S)-6-(6-((13-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)tridecyl)carbamoyl)pyridin- |
| | 3-yl)-2,3-dihydrobenzofuran-3-yl)-N,1-dimethyl-1H-pyrazolo[4,3-c]quinoline-8-carboxamide |
| A20 | 4-amino-N-((S)-6-(6-((14-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)tetradecyl)carbamoyl)pyridin-3-yl)- |
| | 2,3-dihydrobenzofuran-3-yl)-N-methyl-1,3-dihydrofuro[3,4-c][1,7]naphthyridine-8-carboxamide |
| A21 | 4-amino-7-fluoro-N-((S)-6-(6-((14-(2-(((2S,4R)-1-((S)-2-(1-fluorocyclopropane-1-carboxamido)-3,3-dimethyl- |
| | butanoyl)-4-hydroxypyrrolidine-2-carboxamido)methyl)-5-(4-methylthiazol-5-yl)phenoxy)tetradecyl)carbamoyl) |
| | pyridin-3-yl)-2,3-dihydrobenzofuran-3-yl)-N-methyl-1,3-dihydrofuro[3,4-c]quinoline-8-carboxamide |
| A22 | 2-amino-N-((S)-6-(6-((14-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)tetradecyl)carbamoyl)pyridin-3-yl)- |
| | 2,3-dihydrobenzofuran-3-yl)-N,3-dimethylquinoline-6-carboxamide |
| A23 | 4-amino-N-((S)-6-(6-((3-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)propyl)carbamoyl) |
| | pyridin-3-yl)-2,3-dihydrobenzofuran-3-yl)-N,1-dimethyl-1H-pyrazolo[4,3-c]quinoline-8-carboxamide |
| A24 | 4-amino-N-((S)-6-(6-((5-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)pentyl)carbamoyl) |
| | pyridin-3-yl)-2,3-dihydrobenzofuran-3-yl)-N,1-dimethyl-1H-pyrazolo[4,3-c]quinoline-8-carboxamide |
| A25 | 4-amino-N-((S)-6-(6-((2-(2-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)ethoxy)ethyl)(methyl) |
| | carbamoyl)pyridin-3-yl)-2,3-dihydrobenzofuran-3-yl)-N,1-dimethyl-1H-pyrazolo[4,3-c]quinoline-8-carboxamide |
| A26 | 4-amino-N-((S)-6-(6-((2-((5-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)pentyl)oxy)ethyl)carbamoyl)pyridin- |
| | 3-yl)-2,3-dihydrobenzofuran-3-yl)-N,1-dimethyl-1H-pyrazolo[4,3-c]quinoline-8-carboxamide |
| A27 | 4-amino-N-((S)-6-(6-((2-(2-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)ethoxy)ethyl)carbamoyl) |
| | pyridin-3-yl)-2,3-dihydrobenzofuran-3-yl)-N-methyl-1,3-dihydrofuro[3,4-c][1,7]naphthyridine-8-carboxamide |
| A28 | 4-amino-N-((S)-6-(6-((4-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethoxy)butyl)carbamoyl)pyridin- |
| | 3-yl)-2,3-dihydrobenzofuran-3-yl)-N,1-dimethyl-1H-pyrazolo[4,3-c]quinoline-8-carboxamide |
| A29 | 4-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N,1-dimethyl- |
| | 1H-pyrazolo[4,3-c]quinoline-8-carboxamide |
| A30 | 4-amino-N-((S)-6-(6-(4-(2-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethoxy)ethyl)piperazine-1- |
| | carbonyl)pyridin-3-yl)-2,3-dihydrobenzofuran-3-yl)-N,1-dimethyl-1H-pyrazolo[4,3-c]quinoline-8-carboxamide |
| A31 | N1-(6-amino-5-ethylpyridin-3-yl)-N2-((S)-6-(6-((2-(2-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)ethoxy)ethyl)carbamoyl)pyridin-3-yl)-2,3-dihydrobenzofuran-3-yl)-N2-methyloxalamide |
| A32 | 4-amino-N-((S)-6-(6-(9-(2-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)ethyl)-3,9-diazaspiro |
| | [5.5]undecane-3-carbonyl)pyridin-3-yl)-2,3-dihydrobenzofuran-3-yl)-N,1-dimethyl- |
| | 1H-pyrazolo[4,3-c]quinoline-8-carboxamide |
| A33 | N1-(6-amino-5-ethylpyridin-3-yl)-N2-((S)-6-(6-((8-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)octyl) |
| | carbamoyl)pyridin-3-yl)-2,3-dihydrobenzofuran-3-yl)-N2-methyloxalamide |
| A34 | N1-(6-amino-5-methylpyridin-3-yl)-N2-((S)-6-(6-((2-(2-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)ethoxy)ethyl)carbamoyl)pyridin-3-yl)-2,3-dihydrobenzofuran-3-yl)-N2-methyloxalamide |
| A35 | 4-amino-N-((S)-6-(6-((2-((5-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)pentyl)(methyl)amino)ethyl) |
| | carbamoyl)pyridin-3-yl)-2,3-dihydrobenzofuran-3-yl)-N,1-dimethyl-1H-pyrazolo[4,3-c]quinoline-8-carboxamide |
| A36 | 4-amino-N-((S)-6-(6-(4-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethyl)piperazine-1-carbonyl)pyridin- |
| | 3-yl)-2,3-dihydrobenzofuran-3-yl)-N,1-dimethyl-1H-pyrazolo[4,3-c]quinoline-8-carboxamide |
| A37 | 4-amino-N-((S)-6-(6-(9-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethyl)-3,9-diazaspiro[5.5]undecane-3- |
| | carbonyl)pyridin-3-yl)-2,3-dihydrobenzofuran-3-yl)-N,1-dimethyl-1H-pyrazolo[4,3-c]quinoline-8-carboxamide |
| A38 | 4-amino-N-((S)-6-(6-((2-((5-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)pentyl)(methyl)amino)ethyl) |
| | carbamoyl)pyridin-3-yl)-2,3-dihydrobenzofuran-3-yl)-N-methyl-1,3-dihydrofuro[3,4-c][1,7]naphthyridine-8- |
| | carboxamide |
| A39 | 4-amino-N-((S)-6-(6-((2-(2-(2-(2-(5-ethynyl-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)ethoxy)ethoxy)ethyl) |
| | carbamoyl)pyridin-3-yl)-2,3-dihydrobenzofuran-3-yl)-N-methyl-1,3-dihydrofuro[3,4-c][1,7]naphthyridine-8- |
| | carboxamide |
| A40 | 2-amino-N-((S)-6-(6-((2-(2-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)ethoxy)ethyl)carbamoyl) |
| | pyridin-3-yl)-2,3-dihydrobenzofuran-3-yl)-N,3-dimethylquinoline-6-carboxamide |
| A41 | 4-amino-3-bromo-N-((S)-6-(6-((2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)ethoxy) |
| | ethyl)carbamoyl)pyridin-3-yl)-2,3-dihydrobenzofuran-3-yl)-N,1-dimethyl-1H-pyrazolo[4,3-c]quinoline-8-carboxamide |
| A42 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N,3-dimethylquinoline-6-carboxamide |
| A43 | 4-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methyl-1,3-dihydrofuro[3,4-c][1,7] |
| | naphthyridine-8-carboxamide |
| A44 | N1-(7-amino-1-methyl-1H-pyrazolo[3,4-c]pyridin-4-yl)-N2-((S)-6-(6-((2-(2-(2-(2-(((2S,4R)-1-((S)-2-(1- |
| | fluorocyclopropane-1-carboxamido)-3,3-dimethylbutanoyl)-4-hydroxypyrrolidine-2-carboxamido)methyl)- |
| | 5-(4-methylthiazol-5-yl)phenoxy)ethoxy)ethoxy)ethyl)carbamoyl)pyridin-3-yl)-2,3-dihydrobenzofuran- |
| | 3-yl)-N2-methyloxalamide |
| A45 | 4-amino-N-((S)-6-(6-((2-(2-(2-(2-(((2S,4R)-1-((S)-2-(1-fluorocyclopropane-1-carboxamido)-3,3-dimethyl- |
| | butanoyl)-4-hydroxypyrrolidine-2-carboxamido)methyl)-5-(4-methylthiazol-5-yl)phenoxy)ethoxy)ethoxy) |
| | ethyl)carbamoyl)pyridin-3-yl)-2,3-dihydrobenzofuran-3-yl)-N-isopropyl-1,3-dihydrofuro[3,4-c][1,7] |
| | naphthyridine-8-carboxamide |
| A46 | 2-amino-N-((S)-6-(6-((2-(2-(2-(2-(5-ethynyl-2-(((2S,4R)-1-((S)-2-(1-fluorocyclopropane-1-carboxamido)- |
| | 3,3-dimethylbutanoyl)-4-hydroxypyrrolidine-2-carboxamido)methyl)phenoxy)ethoxy)ethoxy)ethoxy)ethyl) |
| | carbamoyl)pyridin-3-yl)-2,3-dihydrobenzofuran-3-yl)-N,3-dimethylquinoline-6-carboxamide |
| A47 | 2-amino-3-cyclopropyl-N-((S)-6-(6-((2-(2-(2-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy)ethoxy) |
| | ethoxy)ethyl)carbamoyl)pyridin-3-yl)-2,3-dihydrobenzofuran-3-yl)-N-methylquinoline-6-carboxamide |
| A48 | 2-amino-N-((S)-6-(6-((2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)ethoxy)ethyl)carbamoyl)pyridin-3-yl)-2,3-dihydrobenzofuran-3-yl)-N-isopropyl-3- |
| | methylquinoline-6-carboxamide |
| A49 | 2-amino-3-cyclopropyl-N-((S)-6-(6-(((1S,4S)-4-(2-(2-(((2S,4R)-1-((S)-2-(1-fluorocyclopropane- |
| | 1-carboxamido)-3,3-dimethylbutanoyl)-4-hydroxypyrrolidine-2-carboxamido)methyl)-5-(4- |
| | methylthiazol-5-yl)phenoxy)ethoxy)cyclohexyl)carbamoyl)pyridin-3-yl)-2,3-dihydrobenzofuran- |
| | 3-yl)-N-methylquinoline-6-carboxamide |
| A50 | 4-amino-N-ethyl-N-((S)-6-(6-((2-(2-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4- |
| | methylthiazol-5-yl)phenoxy)ethoxy)ethoxy)ethyl)carbamoyl)pyridin-3-yl)-2,3-dihydrobenzofuran- |
| | 3-yl)-1,3-dihydrofuro[3,4-c][1,7]naphthyridine-8-carboxamide |
| A51 | 2-amino-3-cyclopropyl-N-(cyclopropylmethyl)-N-((S)-6-(6-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2- |
| | carboxamido)methyl)-5-(4-methylthiazol-5-yl)phenoxy)ethoxy)cyclohexyl)carbamoyl)pyridin-3- |
| | yl)-2,3-dihydrobenzofuran-3-yl)quinoline-6-carboxamide |
| A52 | 4-amino-N-((S)-6-(6-((2-(2-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5- |
| | yl)phenoxy)ethoxy)ethoxy)ethyl)carbamoyl)pyridin-3-yl)-2,3-dihydrobenzofuran-3-yl)-N,1,3- |
| | trimethyl-1H-pyrazolo[4,3-c]quinoline-8-carboxamide |
| A53 | N1-(7-amino-1-methyl-1H-pyrazolo[3,4-c]pyridin-4-yl)-N2-((S)-6-(6-(((1S,4S)-4-(2-(2-(((2S,4R)- |
| | 1-((S)-2-(1-fluorocyclopropane-1-carboxamido)-3,3-dimethylbutanoyl)-4-hydroxypyrrolidine-2- |
| | carboxamido)methyl)-5-(4-methylthiazol-5-yl)phenoxy)ethoxy)cyclohexyl)carbamoyl)pyridin- |
| | 3-yl)-2,3-dihydrobenzofuran-3-yl)-N2-methyloxalamide |
| A54 | 2-amino-N-(cyclopropylmethyl)-N-((S)-6-(6-(((1S,4S)-4-(2-(2-(((2S,4R)-1-((S)-2-(1-fluorocyclo- |
| | propane-1-carboxamido)-3,3-dimethylbutanoyl)-4-hydroxypyrrolidine-2-carboxamido)methyl)- |
| | 5-(4-methylthiazol-5-yl)phenoxy)ethoxy)cyclohexyl)carbamoyl)pyridin-3-yl)-2,3- |
| | dihydrobenzofuran-3-yl)-3-methylquinoline-6-carboxamide |
| A55 | 4-amino-N-((S)-6-(6-(((1S,4S)-4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5- |
| | yl)phenoxy)ethoxy)cyclohexyl)carbamoyl)pyridin-3-yl)-2,3-dihydrobenzofuran-3-yl)-N,1,3- |
| | trimethyl-1H-pyrazolo[4,3-c]quinoline-8-carboxamide |
| A56 | N1-(7-amino-1-methyl-1H-pyrazolo[3,4-c]pyridin-4-yl)-N2-(cyclopropylmethyl)-N2-((S)-6-(6- |
| | (((1S,4S)-4-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)cyclohexyl) |
| | carbamoyl)pyridin-3-yl)-2,3-dihydrobenzofuran-3-yl)oxalamide |
| A57 | 2-amino-3-cyclopropyl-N-((S)-6-(6-((17-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)- |
| | 3,6,9,12,15-pentaoxaheptadecyl)carbamoyl)pyridin-3-yl)-2,3-dihydrobenzofuran-3-yl)-N-methylquinoline- |
| | 6-carboxamide |
| A58 | 2-amino-3-cyclopropyl-N-((S)-6-(6-(((1S,4S)-4-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy)cyclohexyl) |
| | carbamoyl)pyridin-3-yl)-2,3-dihydrobenzofuran-3-yl)-N-methylquinoline-6-carboxamide |
| A59 | 2-amino-N-((S)-6-(6-((17-(5-ethynyl-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)-3,6,9,12,15-pentaoxaheptadecyl) |
| | carbamoyl)pyridin-3-yl)-2,3-dihydrobenzofuran-3-yl)-N,3-dimethylquinoline-6-carboxamide |
| A60 | 2-amino-N-((S)-6-(6-(((1S,4S)-4-(2-(5-ethynyl-2-(((2S,4R)-1-((S)-2-(1-fluorocyclopropane-1-carboxamido)- |
| | 3,3-dimethylbutanoyl)-4-hydroxypyrrolidine-2-carboxamido)methyl)phenoxy)ethoxy)cyclohexyl)carbamoyl) |
| | pyridin-3-yl)-2,3-dihydrobenzofuran-3-yl)-N,3-dimethylquinoline-6-carboxamide |
| A61 | 4-amino-3-bromo-N-((S)-6-(6-(((1S,4S)-4-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)cyclohexyl)carbamoyl)pyridin-3-yl)-2,3-dihydrobenzofuran-3-yl)-N,1-dimethyl-1H-pyrazolo |
| | [4,3-c]quinoline-8-carboxamide |
| A62 | N-((S)-6-(6-(((1S,4S)-4-(2-(2-(((2S,4R)-1-((S)-2-acetamido-3,3-dimethylbutanoyl)-4-hydroxypyrrolidine-2- |
| | carboxamido)methyl)-5-(4-methylthiazol-5-yl)phenoxy)ethoxy)cyclohexyl)carbamoyl)pyridin-3-yl)-2,3- |
| | dihydrobenzofuran-3-yl)-2-amino-N,3-dimethylquinoline-6-carboxamide |
| A63 | 2-amino-3-bromo-N-((S)-6-(6-(((1S,4S)-4-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy) |
| | cyclohexyl)carbamoyl)pyridin-3-yl)-2,3-dihydrobenzofuran-3-yl)-N-methylquinoline-6-carboxamide |
| A64 | 2-amino-3-cyclopropyl-N-ethyl-N-((S)-6-(6-(((1S,4S)-4-(2-(5-ethynyl-2-(((2S,4R)-1-((S)-2-(1-fluorocyclopropane- |
| | 1-carboxamido)-3,3-dimethylbutanoyl)-4-hydroxypyrrolidine-2-carboxamido)methyl)phenoxy)ethoxy) |
| | cyclohexyl)carbamoyl)pyridin-3-yl)-2,3-dihydrobenzofuran-3-yl)quinoline-6-carboxamide |
| A65 | N-((S)-6-(6-(((1S,4S)-4-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethoxy)cyclohexyl)carbamoyl)pyridin- |
| | 3-yl)-2,3-dihydrobenzofuran-3-yl)-N,3-dimethylquinoline-6-carboxamide |
| A66 | N1-(7-amino-1-ethyl-1H-pyrazolo[3,4-c]pyridin-4-yl)-N2-ethyl-N2-((S)-6-(6-((2-(2-(2-(2-(((2S,4R)-1-((S)-2-(1- |
| | fluorocyclopropane-1-carboxamido)-3,3-dimethylbutanoyl)-4-hydroxypyrrolidine-2-carboxamido)methyl)-5-(4- |
| | methylthiazol-5-yl)phenoxy)ethoxy)ethoxy)ethyl)carbamoyl)pyridin-3-yl)-2,3-dihydrobenzofuran-3-yl)oxalamide |
| A67 | 2-amino-N-(cyclopropylmethyl)-N-((S)-6-(6-(((1S,4S)-4-(2-(5-ethynyl-2-(((2S,4R)-1-((S)-2-(1- |
| | fluorocyclopropane-1-carboxamido)-3,3-dimethylbutanoyl)-4-hydroxypyrrolidine-2-carboxamido)methyl) |
| | phenoxy)ethoxy)cyclohexyl)carbamoyl)pyridin-3-yl)-2,3-dihydrobenzofuran-3-yl)-3-methylquinoline-6-carboxamide |
| A68 | 2-amino-3-bromo-N-((S)-6-(6-(((1S,4S)-4-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy)cyclohexyl) |
| | carbamoyl)pyridin-3-yl)-2,3-dihydrobenzofuran-3-yl)-N-isopropylquinoline-6-carboxamide |
| A69 | N-(cyclopropylmethyl)-N-((S)-6-(6-(((1S,4S)-4-(2-(2-(((2S,4R)-1-((S)-2-(1-fluorocyclopropane- |
| | 1-carboxamido)-3,3-dimethylbutanoyl)-4-hydroxypyrrolidine-2-carboxamido)methyl)-5-(4- |
| | methylthiazol-5-yl)phenoxy)ethoxy)cyclohexyl)carbamoyl)pyridin-3-yl)-2,3-dihydrobenzofuran- |
| | 3-yl)-3-methylquinoline-6-carboxamide |
| A70 | 2-amino-3-cyclopropyl-N-((S)-6-(6-((2-(2-(2-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5-yl) |
| | phenoxy)ethoxy)ethoxy)ethoxy)ethyl)carbamoyl)pyridin-3-yl)-2,3-dihydrobenzofuran-3-yl)-N-methylquinoline-6- |
| | carboxamide |
| A71 | 2-amino-3-ethynyl-N-((S)-6-(6-(((1S,4S)-4-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy)cyclohexyl) |
| | carbamoyl)pyridin-3-yl)-2,3-dihydrofuran-3-yl)-N-methylquinoline-6-carboxamide |
| A72 | 2-amino-N-((S)-6-(6-(((1S,4S)-4-(2-(5-ethynyl-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)cyclohexyl)carbamoyl)pyridin-3-yl)- |
| | 2,3-dihydrobenzofuran-3-yl)-3-isopropyl-N-methylquinoline-6-carboxamide |
| A73 | 2-amino-3-cyclopropyl-N-((S)-6-(6-((2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)ethoxy) |
| | ethyl)carbamoyl)pyridin-3-yl)-2,3-dihydrobenzofuran-3-yl)-N-methylquinoline-6-carboxamide |
| A74 | N-((S)-6-(6-(((1S,4S)-4-(2-(5-ethynyl-2-(((2S,4R)-1-((S)-2-(1-fluorocyclopropane-1-carboxamido)- |
| | 3,3-dimethylbutanoyl)-4-hydroxypyrrolidine-2-carboxamido)methyl)phenoxy)ethoxy)cyclohexyl) |
| | carbamoyl)pyridin-3-yl)-2,3-dihydrobenzofuran-3-yl)-N,3-dimethylquinoline-6-carboxamide |
| A75 | 2-amino-3-cyclopropyl-N-((S)-6-(6-((4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy) |
| | butyl)carbamoyl)pyrrolidin-3-yl)-2,3-dihydrobenzofuran-3-yl)-N-methylquinoline-6-carboxamide |
| A76 | 3-cyclopropyl-N-(cyclopropylmethyl)-N-((S)-6-(6-(((1S,4S)-4-(2-(2-(((2S,4R)-1-((S)-2-(1-fluorocyclopropane- |
| | 1-carboxamido)-3,3-dimethylbutanoyl)-4-hydroxypyrrolidine-2-carboxamido)methyl)-5-(4-methylthiazol-5- |
| | yl)phenoxy)ethoxy)cyclohexyl)carbamoyl)pyridin-3-yl)-2,3-dihydrobenzofuran-3-yl)quinoline-6-carboxamide |
| A77 | 2-amino-N-((S)-6-(6-((2-(2-(2-(2-(5-ethynyl-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)ethoxy)ethoxy)ethyl) |
| | carbamoyl)pyridin-3-yl)-2,3-dihydrobenzofuran-3-yl)-3-isopropyl-N-methylquinoline-6-carboxamide |
| A78 | 2-amino-3-cyclopropyl-N-((S)-6-(6-(((1S,4S)-4-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy)cyclohexyl) |
| | carbamoyl)pyridin-3-yl)-2,3-dihydrobenzofuran-3-yl)-N-isopropylquinoline-6-carboxamide |
| A79 | N1-(7-amino-1-isopropyl-1H-pyrazolo[3,4-c]pyridin-4-yl)-N2-((S)-6-(6-((2-(2-(2-(2-(((2S,4R)-1-((S)-2-(1- |
| | fluorocyclopropane-1-carboxamido)-3,3-dimethylbutanoyl)-4-hydroxypyrrolidine-2-carboxamido)methyl)-5-(4- |
| | methylthiazol-5-yl)phenoxy)ethoxy)ethoxy)ethyl)carbamoyl)pyridin-3-yl)-2,3-dihydrobenzofuran-3-yl)-N2- |
| | methyloxalamide |
| A80 | 2-amino-3-cyclopropyl-N-((S)-6-(1-(2-(2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)ethoxy) |
| | ethoxy)ethyl)-1H-pyrazol-4-yl)-2,3-dihydrobenzofuran-3-yl)-N-methylquinoline-6-carboxamide |
| A81 | 4-amino-N-((S)-6-(1-(2-(2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | ethoxy)ethoxy)ethyl)-1H-pyrazol-4-yl)-2,3-dihydrobenzofuran-3-yl)-N,1-dimethyl-1H-pyrazolo[4,3-c] |
| | quinoline-8-carboxamide |
| A82 | 2-amino-3-cyclopropyl-N-((S)-6-(6-((4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | butyl)carbamoyl)pyridin-3-yl)-2,3-dihydrobenzofuran-3-yl)-N-isopropylquinoline-6-carboxamide |
| A83 | 2-amino-3-chloro-N-((S)-6-(6-(((1S,4S)-4-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy)cyclohexyl) |
| | carbamoyl)pyridin-3-yl)-2,3-dihydrobenzofuran-3-yl)-N-methylquinoline-6-carboxamide |
| A84 | 2-amino-3-ethynyl-N-((S)-6-(6-(((1S,4S)-4-(2-(2-(((2S,4S)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)cyclohexyl) |
| | carbamoyl)pyridin-3-yl)-2,3-dihydrobenzofuran-3-yl)-N-methylquinoline-6-carboxamide |
| A85 | 2-amino-3-cyclopropyl-N-((S)-6-(6-((4-(2-(5-ethynyl-2-(((2S,4R)-1-((S)-2-(1-fluorocyclopropane-1-carboxamido)- |
| | 3,3-dimethylbutanoyl)-4-hydroxypyrrolidine-2-carboxamido)methyl)phenoxy)ethoxy)butyl)carbamoyl)pyridin-3-yl)-2,3- |
| | dihydrobenzofuran-3-yl)-N-methylquinoline-6-carboxamide |
| A86 | 2-amino-N-((S)-6-(6-((4-(2-(5-ethynyl-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)butyl)carbamoyl) |
| | pyridin-3-yl)-2,3-dihydrobenzofuran-3-yl)-N-isopropyl-3-methylquinoline-6-carboxamide |
| A87 | 2-amino-N-((S)-6-(6-((2-(2-(2-(5-ethynyl-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)ethoxy)ethyl)carbamoyl) |
| | pyridin-3-yl)-2,3-dihydrobenzofuran-3-yl)-N-isopropyl-3-methylquinoline-6-carboxamide |
| A88 | 2-amino-N-((S)-6-(6-(((1S,4S)-4-(2-(5-ethynyl-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)cyclohexyl)carbamoyl)pyridin-3- |
| | yl)-2,3-dihydrobenzofuran-3-yl)-N-methyl-3-(tetrahydro-2H-pyran-4-yl)quinoline-6-carboxamide |
| A89 | 2-amino-3-cyclopropyl-N-((S)-6-(6-((2-(2-(2-(5-ethynyl-2-(((2S,4R)-1-((S)-2-(1-fluorocyclopropane-1-carboxamido)- |
| | 3,3-dimethylbutanoyl)-4-hydroxypyrrolidine-2-carboxamido)methyl)phenoxy)ethoxy)ethoxy)ethyl)carbamoyl)pyridin-3- |
| | yl)-2,3-dihydrobenzofuran-3-yl)-N-methylquinoline-6-carboxamide |
| A90 | 2-amino-7-fluoro-N-((S)-6-(6-((2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)ethoxy) |
| | ethyl)carbamoyl)pyridin-3-yl)-2,3-dihydrobenzofuran-3-yl)-3-(hydroxymethyl)-N,4-dimethylquinoline-6-carboxamide |
| A91 | 2-amino-3-cyano-N-((S)-6-(6-(((1S,4S)-4-(2-(5-ethynyl-2-(((2S,4R)-1-((S)-2-(1-fluorocyclopropane-1-carboxamido)- |
| | 3,3-dimethylbutanoyl)-4-hydroxypyrrolidine-2-carboxamido)methyl)phenoxy)ethoxy)cyclohexyl)carbamoyl)pyridin-3- |
| | yl)-2,3-dihydrobenzofuran-3-yl)-N-methylquinoline-6-carboxamide |
| A92 | 2-amino-3-cyclobutyl-N-((S)-6-(6-(((1S,4S)-4-(2-(5-ethynyl-2-(((2S,4R)-1-((S)-2-(1-fluorocyclopropane-1- |
| | carboxamido)-3,3-dimethylbutanoyl)-4-hydroxypyrrolidine-2-carboxamido)methyl)phenoxy)ethoxy)cyclohexyl) |
| | carbamoyl)pyridin-3-yl)-2,3-dihydrobenzofuran-3-yl)-N-methylquinoline-6-carboxamide |
| A93 | N1-(6-amino-5-methylpyridin-3-yl)-N2-((S)-6-(6-((2-(2-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)ethoxy)ethyl)carbamoyl)pyridin-3-yl)-2,3-dihydrobenzofuran-3-yl)-N2-isopropyloxalamide |
| A94 | 2-amino-N-((S)-6-(6-((2-(2-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)ethoxy)ethyl)carbamoyl)pyridin- |
| | 3-yl)-2,3-dihydrobenzofuran-3-yl)-N-isobutyl-3-methylquinoline-6-carboxamide |
| A95 | 2-amino-N-cyclopentyl-N-((S)-6-(6-((2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)ethoxy) |
| | ethyl)carbamoyl)pyridin-3-yl)-2,3-dihydrobenzofuran-3-yl)-3-methylquinoline-6-carboxamide |
| A96 | 2-amino-N-cyclobutyl-N-((S)-6-(6-((2-(2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-4-yl)phenoxy)ethoxy)ethoxy) |
| | ethyl)carbamoyl)pyridin-3-yl)-2,3-dihydrobenzofuran-3-yl)-3-methylquinoline-6-carboxamide |
| A97 | 2-amino-3-cyclobutyl-N-((S)-6-(6-((4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)butyl) |
| | carbamoyl)pyridin-3-yl)-2,3-dihydrobenzofuran-3-yl)-N-methylquinoline-6-carboxamide |
| A98 | 2-amino-3-cyano-N-((S)-6-(6-((4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)butyl) |
| | carbamoyl)pyridin-3-yl)-2,3-dihydrobenzofuran-3-yl)-N-methylquinoline-6-carboxamide |
| A99 | N1-(6-amino-5-methylpyridin-3-yl)-N2-((S)-6-(6-((4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy)butyl) |
| | carbamoyl)pyridin-3-yl)-2,3-dihydrobenzofuran-3-yl)-N2-isopropyloxalamide |
| A100 | N1-(6-amino-5-ethylpyridin-3-yl)-N2-((S)-6-(6-((2-(2-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy)ethoxy) |
| | ethyl)carbamoyl)pyridin-3-yl)-2,3-dihydrobenzofuran-3-yl)-N2-isopropyloxalamide |
| A101 | 2-amino-N-(cyclopropylmethyl)-N-((S)-6-(6-((2-(2-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)ethoxy)ethyl)carbamoyl)pyridin-3-yl)-2,3-dihydrobenzofuran-3-yl)-3-methylquinoline-6-carboxamide |
| A102 | N1-(6-amino-5-ethylpyridin-3-yl)-N2-((S)-6-(6-((4-(2-(2-(((2S,4R)-1-((S)-2-(1- |
| | fluorocyclopropane-1-carboxamido)-3,3-dimethylbutanoyl)-4-hydroxypyrrolidine-2- |
| | carboxamido)methyl)-5-(4-methylthiazol-5-yl)phenoxy)ethoxy)butyl)carbamoyl)pyridin-3-yl)-2,3- |
| | dihydrobenzofuran-3-yl)-N2-isopropyloxalamide |
| A103 | 2-amino-3-bromo-N-((S)-6-(6-((4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy) |
| | ethoxy)butyl)carbamoyl)pyridin-3-yl)-2,3-dihydrobenzofuran-3-yl)-N-methylquinoline-6-carboxamide |
| A104 | 2-amino-3-(tert-butyl)-N-((S)-6-(6-((4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy) |
| | butyl)carbamoyl)pyridin-3-yl)-2,3-dihydrobenzofuran-3-yl)-N-methylquinoline-6-carboxamide |
| A105 | N1-(6-amino-5-ethylpyridin-3-yl)-N2-((S)-6-(6-((4-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)butyl)carbamoyl)pyridin-3-yl)-2,3-dihydrobenzofuran-3-yl)-N2-methyloxalamide |
| A106 | N-((S)-6-(6-((4-(2-(5-ethynyl-2-(((2S,4R)-1-((S)-2-(1-fluorocyclopropane-1-carboxamido)-3,3-dimethylbutanoyl)- |
| | 4-hydroxypyrrolidine-2-carboxamido)methyl)phenoxy)ethoxy)butyl)carbamoyl)pyridin-3-yl)-2,3- |
| | dihydrobenzofuran-3-yl)-N,3-dimethylquinoline-6-carboxamide |
| A107 | N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl) |
| | piperazine-1-carbonyl)pyridin-3-yl)-2,3-dihydrobenzofuran-3-yl)-N,3-dimethylquinoline-6-carboxamide |
| A108 | N-((S)-6-(6-(4-((4-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)-3,6-dihydropyridin-1(2H)- |
| | yl)methyl)piperidine-1-carbonyl)pyridin-3-yl)-2,3-dihydrobenzofuran-3-yl)-N,3-dimethylquinoline-6-carboxamide |
| A109 | 2-amino-N-((S)-6-(6-(((1-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethyl)piperidin-4-yl) |
| | methyl)carbamoyl)pyridin-3-yl)-2,3-dihydrobenzofuran-3-yl)-N,3-dimethylquinoline-6-carboxamide |
| A110 | 4-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl) |
| | piperazine-1-carbonyl)pyridin-3-yl)-2,3-dihydrobenzofuran-3-yl)-N,1-dimethyl-1H-pyrazolo[4,3-c] |
| | quinoline-8-carboxamide |
| A111 | 4-amino-N-((S)-6-(6-(4-((1-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)methyl)piperazine-1- |
| | carbonyl)pyridin-3-yl)-2,3-dihydrobenzofuran-3-yl)-N-methyl-1,3-dihydrofuro[3,4-c][1,7]naphthyridine-8- |
| | carboxamide |
| A112 | N-((S)-6-(6-(((1S,4S)-4-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethoxy)cyclohexyl)carbamoyl) |
| | pyridin-3-yl)-2,3-dihydrobenzofuran-3-yl)-N-methylquinoline-6-carboxamide |
| A113 | 4-amino-N-((S)-6-(6-(4-((1-(5-fluoro-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenyl)piperidin-4-yl)methyl)piperazine-1- |
| | carbonyl)pyridin-3-yl)-2,3-dihydrobenzofuran-3-yl)-N,1-dimethyl-1H-pyrazolo[4,3-c]quinoline-8-carboxamide |
| A114 | 3-cyclopropyl-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N-methylquinoline-6-carboxamide |
| A115 | N-((S)-6-(6-(4-((1-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N-methylquinoline-6-carboxamide |
| A116 | 2-amino-N-((S)-6-(6-(((1-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethyl) |
| | piperidin-4-yl)methyl)carbamoyl)pyridin-3-yl)-2,3-dihydrobenzofuran-3-yl)-N-isopropyl-3-methylquinoline- |
| | 6-carboxamide |
| A117 | 4-amino-N-((S)-6-(6-(6-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)-2,6- |
| | diazaspiro[3.3]heptane-2-carbonyl)pyridin-3-yl)-2,3-dihydrobenzofuran-3-yl)-N,1-dimethyl-1H-pyrazolo |
| | [4,3-c]quinoline-8-carboxamide |
| A118 | N1-(7-amino-1-methyl-1H-pyrazolo[3,4-c]pyridin-4-yl)-N2-((S)-6-(6-(((1S,4S)-4-(2-(2-(((2S,4S)-1-((S)-2- |
| | (1-fluorocyclopropane-1-carboxamido)-3,3-dimethylbutanoyl)-4-hydroxypyrrolidine-2-carboxamido)methyl)- |
| | 5-(4-methylthiazol-5-yl)phenoxy)ethoxy)cyclohexyl)carbamoyl)yl)pyridin-3-yl)-2,3-dihydrobenzofuran-3-yl)- |
| | N2-methyloxalamide |
| A119 | 4-amino-N-((S)-6-(6-(7-((1-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)methyl)-2,7- |
| | diazaspiro[3.5]nonane-2-carbonyl)pyridin-3-yl)-2,3-dihydrobenzofuran-3-yl)-N,1-dimethyl-1H-pyrazolo |
| | [4,3-c]quinoline-8-carboxamide |
| A120 | 4-amino-N-((S)-6-(6-(4-((1-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)methyl)piperazine-1- |
| | carbonyl)pyridin-3-yl)-2,3-dihydrobenzofuran-3-yl)-N-isopropyl-1,3-dihydrofuro[3,4-c][1,7]naphthyridine-8- |
| | carboxamide |
| A121 | 4-amino-N-((S)-6-(6-(4-((1-(5-chloro-2-(((2S,4R)-1-((S)-2-(1-fluorocyclopropane-1-carboxamido)-3,3-dimethylbutanoyl)-4- |
| | hydroxypyrrolidine-2-carboxamido)methyl)phenyl)piperidin-4-yl)methyl)piperazine-1-carbonyl)pyridin-3-yl)-2,3- |
| | dihydrobenzofuran-3-yl)-N,1-dimethyl-1H-pyrazolo[4,3-c]quinoline-8-carboxamide |
| A122 | 4-amino-N-((S)-6-(6-(4-((1-(2-((S)-1-((2S,4R)-1-((S)-2-(1-fluorocyclopropane-1-carboxamido)-3,3-dimethylbutanoyl)-4- |
| | hydroxypyrrolidine-2-carboxamido)ethyl)-5-(4-methylthiazol-5-yl)phenyl)piperidin-4-yl)methyl) |
| | piperazine-1-carbonyl)pyridin-3-yl)-2,3-dihydrobenzofuran-3-yl)-N,1-dimethyl-1H-pyrazolo[4,3-c] |
| | quinoline-8-carboxamide |
| A123 | 4-amino-N-((S)-6-(6-(6-((1-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)methyl)-2,6- |
| | diazaspiro[3.4]octane-2-carbonyl)pyridin-3-yl)-2,3-dihydrobenzofuran-3-yl)-N,1-dimethyl-1H-pyrazolo |
| | [4,3-c]quinoline-8-carboxamide |
| A124 | 4-amino-N-((S)-6-(6-(((1-(3-(2-(((2S,4R)-1-((S)-2-(1-fluorocyclopropane-1-carboxamido)-3,3-dimethybutanoyl)-4- |
| | hydroxypyrrolidine-2-carboxamido)methyl)-5-(4-methylthiazol-5-yl)phenoxy)propyl)piperidin-4-yl) |
| | carbamoyl)pyridin-3-yl)-2,3-dihydrobenzofuran-3-yl)-N,1-dimethyl-1H-pyrazolo[4,3-c]quinoline-8-carboxamide |
| A125 | 4-amino-N-((S)-6-(6-(((R)-1-(3-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)propyl)pyrrolidin-3-yl)carbamoyl) |
| | pyridin-3-yl)-2,3-dihydrobenzofuran-3-yl)-N,1-dimethyl-1H-pyrazolo[4,3-c]quinoline-8-carboxamide |
| A126 | 2-amino-N-((S)-6-(6-((4-(4-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperazin-1-yl)butyl)carbamoyl) |
| | pyridin-3-yl)-2,3-dihydrobenzofuran-3-yl)-N,3-dimethylquinoline-6-carboxamide |
| A127 | N-(cyclopropylmethyl)-N-((S)-6-(4-(((1S,4S)-4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy) |
| | cyclohexyl)carbamoyl)phenyl)-2,3-dihydrobenzofuran-3-yl)-3-methylquinoline-6-carboxamide |
| A128 | 4-amino-N-((S)-6-(6-((7-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)heptyl)carbamoyl)pyridin-3- |
| | yl)-2,3-dihydrobenzofuran-3-yl)-N,1-dimethyl-1H-pyrazolo[4,3-c]quinoline-8-carboxamide |
| A129 | N-(cyclopropylmethyl)-N-((S)-6-(6-((1-(3-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)propyl) |
| | piperidin-4-yl)carbamoyl)pyridin-3-yl)-2,3-dihydrobenzofuran-3-yl)-3-methylquinoline-6-carboxamide |
| A130 | 4-amino-N-((S)-6-(6-(((1S,4S)-4-(ethyl(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethyl)amino) |
| | cyclohexyl)carbamoyl)pyridin-3-yl)-2,3-dihydrobenzofuran-3-yl)-N,1-dimethyl-1H-pyrazolo[4,3-c]quinoline- |
| | 8-carboxamide |
| A131 | 2-amino-N-((S)-6-(6-((4-(4-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperazin-1-yl)butyl)carbamoyl) |
| | pyridin-3-yl)-2,3-dihydrobenzofuran-3-yl)-N-isopropyl-3-methylquinoline-6-carboxamide |
| A132 | 4-amino-N-((S)-6-(6-((6-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)hexyl)carbamoyl)pyridin-3- |
| | yl)-2,3-dihydrobenzofuran-3-yl)-N,1-dimethyl-1H-pyrazolo[4,3-c]quinoline-8-carboxamide |
| A133 | 4-amino-N-((S)-6-(6-((5-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)pentyl)carbamoyl)pyridin-3- |
| | yl)-2,3-dihydrobenzofuran-3-yl)-N,1-dimethyl-1H-pyrazolo[4,3-c]quinoline-8-carboxamide |
| A134 | 2-amino-N-((S)-6-(6-(4-(4-((2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)methyl)-1H-1,2,3-triazol-1- |
| | yl)piperidine-1-carbonyl)pyridin-3-yl)-2,3-dihydrobenzofuran-3-yl)-N,3-dimethylquinoline-6-carboxamide |
| A135 | N-(cyclopropylmethyl)-N-((S)-6-(6-((1-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethyl) |
| | piperidin-4-yl)carbamoyl)pyridin-3-yl)-2,3-dihydrobenzofuran-3-yl)-3-methylquinoline-6-carboxamide |
| A136 | N-(cyclopropylmethyl)-N-((S)-6-(6-((4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy) |
| | phenyl)carbamoyl)pyridin-3-yl)-2,3-dihydrobenzofuran-3-yl)-3-methylquinoline-6-carboxamide |
| A137 | 2-amino-N-((S)-6-(6-(4-(4-((2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)methyl)phenyl)piperazine-1- |
| | carbonyl)pyridin-3-yl)-2,3-dihydrobenzofuran-3-yl)-N,3-dimethylquinoline-6-carboxamide |
| A138 | 2-amino-N-((3S)-6-(6-(4-((5-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)hexahydropyrrolo[3,4-c]pyrrol- |
| | 2(1H)-yl)methyl)piperidine-1-carbonyl)pyridin-3-yl)-2,3-dihydrobenzofuran-3-yl)-N,3-dimethylquinoline-6- |
| | carboxamide |
| A139 | 2-amino-N-((S)-6-(6-(9-((1-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)methyl)-3,9-diazaspiro |
| | [5.5]undecane-3-carbonyl)pyridin-3-yl)-2,3-dihydrobenzofuran-3-yl)-N,3-dimethylquinoline-6-carboxamide |
| A140 | 2-amino-N-((S)-6-(6-(4-((2′-(((2S,4R)-1-((S)-2-(1-fluorocyclopropane-1-carboxamido)-3,3-dimethylbutanoyl)-4- |
| | hydroxypyrrolidine-2-carboxamido)methyl)-5′-(4-methylthiazol-5-yl)-[1,1′-biphenyl]-4-yl)methyl)piperazine-1- |
| | carbonyl)pyridin-3-yl)-2,3-dihydrobenzofuran-3-yl)-N,3-dimethylquinoline-6-carboxamide |
| A141 | 2-amino-N-((S)-6-(6-(4-(4-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)but-3-yn-1-yl)piperazine-1-carbonyl) |
| | pyridin-3-yl)-2,3-dihydrobenzofuran-3-yl)-N,3-dimethylquinoline-6-carboxamide |
| A142 | 2-amino-N-((S)-6-(6-((4-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethoxy)phenyl)carbamoyl)pyridin-3- |
| | yl)-2,3-dihydrobenzofuran-3-yl)-N,3-dimethylquinoline-6-carboxamide |
| A143 | N1-(5-carbamoylpyridin-3-yl)-N2-((S)-6-(6-(4-((1-(2-(((2S,4R)-1-((S)-2-(1- |
| | fluorocyclopropane-1-carboxamido)-3,3-dimethylbutanoyl)-4-hydroxypyrrolidine-2-carboxamido)methyl)- |
| | 5-(4-methylthiazol-5-yl)phenyl)piperidin-4-yl)methyl)piperazine-1-carbonyl)pyridin-3-yl)-2,3-dihydrobenzofuran- |
| | 3-yl)-N2-methyloxalamide |
| A144 | N-(cyclopropylmethyl)-N-((R)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-3-methylquinoline-6-carboxamide |
| A145 | 4-amino-N-((S)-6-(6-(4-((4-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)cyclohexyl)methyl)piperazine- |
| | 1-carbonyl)pyridin-3-yl)-2,3-dihydrobenzofuran-3-yl)-N-methyl-1,3-dihydrofuro[3,4-c][1,7]naphthyridine- |
| | 8-carboxamide |
| A146 | 2-amino-N-((S)-6-(6-(4-(1-(2-(1-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)ethyl)piperazine-1- |
| | carbonyl)pyridin-3-yl)-2,3-dihydrobenzofuran-3-yl)-N,3-dimethylquinoline-6-carboxamide |
| A147 | 2-amino-N-((S)-6-(6-((4-(2-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)ethoxy)benzyl)carbamoyl)pyridin-3- |
| | yl)-2,3-dihydrobenzofuran-3-yl)-N,3-dimethylquinoline-6-carboxamide |
| A148 | N-(cyclopropylmethyl)-N-((S)-6-(6-(((1S,4S)-4-((2-(2-(((2S,4R)-1-((S)-2-(1- |
| | fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethyl)(methyl)amino)cyclohexyl)carbamoyl)pyridin-3-yl)-2,3- |
| | dihydrobenzofuran-3-yl)-3-methylquinoline-6-carboxamide |
| A149 | N1-(6-amino-5-fluoropyridin-3-yl)-N2-((S)-6-(6-((2-(2-(2-(2-(((2S,4R)-1-((S)-2-(1- |
| | fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethoxy)ethoxy)ethyl)carbamoyl)pyridin-3-yl)-2,3- |
| | dihydrobenzofuran-3-yl)-N2-methyloxalamide |
| A150 | 2-amino-N-((S)-6-(6-((2-(2-((3-(2-(((2S,4R)-1-((S)-2-(1- |
| | fluorocyclopropane-1-carboxamido)-3,3-dimethylbutanoyl)-4-hydroxypyrrolidine-2-carboxamido) |
| | methyl)-5-(4-methylthiazol-5-yl)phenyl)prop-2-yn-1-yl)oxy)ethoxy)ethyl)carbamoyl)pyridin-3-yl)-2,3- |
| | dihydrobenzofuran-3-yl)-N,3-dimethylquinoline-6-carboxamide |
| A151 | 2-amino-N-((S)-6-(6-(4-(5-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)pent-4-yn-1-yl)piperazine-1-carbonyl) |
| | pyridin-3-yl)-2,3-dihydrobenzofuran-3-yl)-N,3-dimethylquinoline-6-carboxamide |
| A152 | N-(cyclopropylmethyl)-N-((S)-6-(6-((3-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy) |
| | cyclobutyl)carbamoyl)pyridin-3-yl)-2,3-dihydrobenzofuran-3-yl)-3-methylquinoline-6-carboxamide |
| A153 | 2-amino-N-(cyclopropylmethyl)-N-((S)-6-(6-((3-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)cycobutyl)carbamoyl)pyridin-3-yl)-2,3-dihydrobenzofuran-3-yl)-3-methylquinoline-6- |
| | carboxamide |
| A154 | 2-amino-N-((S)-6-(6-(4-(4-(1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)piperazin-1-yl) |
| | piperidine-1-carbonyl)pyridin-3-yl)-2,3-dimethylquinoline-6-carboxamide |
| A155 | N1-(6-amino-5-(difluoromethyl)pyridin-3-yl)-N2-((S)-6-(6-((2-(2-(2-(2-(((2S,4R)-1-((S)-2-(1- |
| | fluorocyclopropane-1-carboxamido)-3,3-dimethylbutanoyl)-4-hydroxypyrrolidine-2-carboxamido)methyl)- |
| | 5-(4-methylthiazol-5-yl)phenoxy)ethoxy)ethoxy)ethyl)carbamoyl)pyridin-3-yl)-2,3-dihydrobenzofuran-3- |
| | yl)-N2-methyloxalamide |
| A156 | 2-amino-N-((R)-6-(6-((1-(3-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenoxy)propyl)piperidin-4-yl)carbamoyl) |
| | pyridin-3-yl)-2,3-dihydrobenzofuran-3-yl)-N,3-dimethylquinoline-6-carboxamide |
| A157 | N1-(6-amino-5-cyanopyridin-3-yl)-N2-((S)-6-(6-(4-((1-(2-(((2S,4R)-1-((S)-2-(1- |
| | fluorocyclopropane-1-carboxamido)-3,3-dimethylbutanoyl)-4-hydroxypyrrolidine-2-carboxamido) |
| | methyl)-5-(4-methylthiazol-5-yl)phenyl)piperidin-4-yl)methyl)piperazine-1-carbonyl)pyridin-3-yl)-2,3- |
| | dihydrobenzofuran-3-yl)-N2-methyloxalamide |
| A158 | 2-amino-N-((S)-6-(6-(4-((4-(1-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenyl) |
| | piperidin-4-yl)piperazin-1-yl)methyl)piperidine-1-carbonyl)pyridin-3-yl)-2,3-dihydrobenzofuran-3-yl)-N,3- |
| | dimethylquinoline-6-carboxamide |
| A159 | 2-amino-N-((S)-6-(6-(2-(1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)-2,7-diazaspiro |
| | [3.5]nonane-7-carbonyl)pyridin-3-yl)-2,3-dihydrobenzofuran-3-yl)-N,3-dimethylquinoline-6-carboxamide |
| A160 | 2-amino-N-((S)-6-(6-(4-(1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)piperazine-1- |
| | carbonyl)pyridin-3-yl)-2,3-dihydrobenzofuran-3-yl)-N,3-dimethylquinoline-6-carboxamide |
| A161 | 4-amino-N-((S)-6-(6-((2-(2-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)ethoxy)ethyl) |
| | carbamoyl)pyridin-3-yl)-2,3-dihydrobenzofuran-3-yl)-N-methylimidazo[1,5-a]quinoxaline-8-carboxamide |
| A162 | 2-amino-N-((R)-6-(6-((1-(3-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)propyl)piperidin-4-yl) |
| | carbamoyl)pyridin-3-yl)-2,3-dihydrobenzofuran-3-yl)-N-isopropyl-3-methylquinoline-6-carboxamide |
| A163 | N1-(8-aminoimidazo[1,5-a]pyrazin-5-yl)-N2-((S)-6-(6-((2-(2-(2-(2-(((2S,4R)-1-((S)-2-(1-fluorocyclopropane- |
| | 1-carboxamido)-3,3-dimethylbutanoyl)-4-hydroxypyrrolidine-2-carboxamido)methyl)-5-(4-methylthiazol- |
| | 5-yl)phenoxy)ethoxy)ethoxy)ethyl)carbamoyl)pyridin-3-yl)-2,3-dihydrobenzofuran-3-yl)-N2-methyloxalamide |
| A164 | 2-amino-N-((R)-6-(6-((3-(4-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)piperidin-1-yl)propyl)carbamoyl) |
| | pyridin-3-yl)-2,3-dihydrobenzofuran-3-yl)-N-isopropyl-3-methylquinoline-6-carboxamide |
| A165 | 2-amino-N-((R)-6-(6-((1-(3-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)-2,2-dimethylpropyl)piperidin-4- |
| | yl)carbamoyl)pyridin-3-yl)-2,3-dihydrobenzofuran-3-yl)-N-isopropyl-3-methylquinoline-6-carboxamide |
| A166 | 2-amino-N-((R)-6-(6-((1′-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)-[1,4′-bipiperidin]-4-yl) |
| | carbamoyl)pyridin-3-yl)-2,3-dihydrobenzofuran-3-yl)-N-isopropyl-3-methylquinoline-6-carboxamide |
| A167 | 2-amino-N-((S)-6-(6-(4-((9-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)-3,9-diazaspiro[5.5]undecan-3- |
| | yl)methyl)piperidine-1-carbonyl)pyridin-3-yl)-2,3-dihydrobenzofuran-3-yl)-N,3-dimethylquinoline-6-carboxamide |
| A168 | 2-amino-3-bromo-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A169 | 2-amino-N-((S)-6-(6-(2-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)-2,7- |
| | diazaspiro[3.5]nonane-7-carbonyl)pyridin-3-yl)-2,3-dihydrobenzofuran-3-yl)-N,3-dimethylquinoline-6-carboxamide |
| A170 | 2-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N-methyl-3-(oxetan-3-yl)quinoline-6-carboxamide |
| A171 | 2-amino-3-cyclopropyl-N-((S)-6-(6-((1-(3-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy) |
| | propyl)piperidin-4-yl)carbamoyl)pyridin-3-yl)-2,3-dihydrobenzofuran-3-yl)-N-methylquinoline-6-carboxamide |
| A172 | 2-amino-3-cyclopropyl-N-((S)-6-(6-(9-(2-(2-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy) |
| | ethoxy)ethoxy)ethyl)-3,9-diazaspiro[5.5]undecane-3-carbonyl)pyridin-3-yl)-2,3-dihydrobenzofuran-3-yl)-N- |
| | methylquinoline-6-carboxamide |
| A173 | 2-amino-3-cyclopropyl-N-((S)-6-(6-(4-((1-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenyl) |
| | piperidin-4-yl)methyl)piperazine-1-carbonyl)pyridin-3-yl)-2,3-dihydrobenzofuran-3-yl)-N-methylquinoline |
| | 6-carboxamide |
| A174 | 2-amino-3-cyano-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin- |
| | 4-yl)methyl)piperazine-1-carbonyl)pyridin-3-yl)-2,3-dihydrobenzofuran-3-yl)-N-methylquinoline-6-carboxamide |
| A175 | 4-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N,1,3-trimethyl-1H-pyrazolo[4,3-c]quinoline-8-carboxamide |
| A176 | 2-amino-3-cyclopropyl-N-((S)-6-(6-(((1S,4S)-4-(ethyl(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethyl) |
| | amino)cyclohexyl)carbamoyl)pyridin-3-yl)-2,3-dihydrobenzofuran-3-yl)-N-methylquinoline-6-carboxamide |
| A177 | N6-((S)-6-(6-(4-((1-(2-(((2S,4R)-1-((S)-2-(1-fluorocyclopropane-1-carboxamido)-3,3-dimethylbutanoyl)-4- |
| | hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N6-methylquinoline-3,6-dicarboxamide |
| A178 | 2-amino-3-cyclopropyl-N-((S)-6-(6-(4-((1-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenyl)piperidin- |
| | 4-yl)methyl)piperazine-1-carbonyl)pyridin-3-yl)-2,3-dihydrobenzofuran-3-yl)-N-isobutylquinoline-6-carboxamide |
| A179 | 2-amino-3-cyclopropyl-N-((S)-6-(6-(4-(4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)but-3-yn-1- |
| | yl)piperazine-1-carbonyl)pyridin-3-yl)-2,3-dihydrobenzofuran-3-yl)-N-methylquinoline-6-carboxamide |
| A180 | 2-amino-3-cyclopropyl-N-((S)-6-(6-(4-((4-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenyl)-3,6- |
| | dihydropyridin-1(2H)-yl)methyl)piperidine-1-carbonyl)pyridin-3-yl)-2,3-dihydrobenzofuran-3-yl)-N- |
| | methylquinoline-6-carboxamide |
| A181 | 3-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl) |
| | piperazine-1-carbonyl)pyridin-3-yl)-2,3-dihydrobenzofuran-3-yl)-N,1-dimethyl-1H-pyrazolo[4,3-c]quinoline-8- |
| | carboxamide |
| A182 | 4-amino-3-cyclopropyl-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methyl-3H-pyrrolo[2,3-c] |
| | quinoline-8-carboxamide |
| A183 | 4-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N-methylimidazo[1,2-a]quinoxaline-8-carboxamide |
| A184 | N1-(8-aminoimidazo[1,5-a]pyrazin-5-yl)-N2-((S)-6-(6-(4-((1-(2-(((2S,4R)-1-((S)-2-(1- |
| | fluorocyclopropane-1-carboxamido)-3,3-dimethylbutanoyl)-4-hydroxypyrrolidine-2-carboxamido)methyl)- |
| | 5-(4-methylthiazol-5-yl)phenyl)piperidin-4-yl)methyl)piperazine-1-carbonyl)pyridin-3-yl)-2,3-dihydrobenzo- |
| | furan-3-yl)-N2-methyloxalamide |
| A185 | 2-amino-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl) |
| | piperidin-4-yl)methyl)piperazine-1-carbonyl)pyridin-3-yl)-2,3-dihydrobenzofuran-3-yl)-N-methyl-3- |
| | (prop-1-yn-1-yl)quinoline-6-carboxamide |
| A186 | 2-amino-3-cyclopropyl-N-((S)-6-(6-(4-((1-(2-((S)-1-((2S,4R)-1-((S)-2-(1-fluorocyclopropane-1-carboxamido)- |
| | 3,3-dimethylbutanoyl)-4-hydroxypyrrolidine-2-carboxamido)ethyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A187 | 4-amino-N-((S)-6-(6-(((1S,4S)-4-((2-(2-(((2S,4R)-1-((S)-2-(1-fluorocyclopropane-1-carboxamido)-3,3-dimethyl- |
| | butanoyl)-4-hydroxypyrrolidine-2-carboxamido)methyl)-5-(4-methylthiazol-5-yl)phenoxy)ethyl)amino)cyclohexyl) |
| | carbamoyl)pyridin-3-yl)-2,3-dihydrobenzofuran-3-yl)-N,1-dimethyl-1H-pyrazolo[4,3-c]quinoline-8-carboxamide |
| A188 | 4-amino-3-cyclopropyl-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methyl-1H-pyrazolo[4,3-c]quinoline- |
| | 8-carboxamide |
| A189 | 4-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N,3-dimethylimidazo[1,5-a]quinoxaline-8-carboxamide |
| A190 | 2-amino-3-cyano-N-((S)-6-(6-(4-((1-(2-((S)-1-((2S,4R)-1-((S)-2-(1-fluorocyclopropane-1-carboxamido)-3,3- |
| | dimethylbutanoyl)-4-hydroxypyrrolidine-2-carboxamido)ethyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A192 | 2-amino-3-cyclopropyl-N-((3S)-6-(6-(5-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4-yl) |
| | methyl)octahydropyrrolo[3,4-c]pyrrole-2-carbonyl)pyridin-3-yl)-2,3-dihydrobenzofuran-3-yl)-N-methylquinoline- |
| | 6-carboxamide |
| A193 | 2-amino-3-cyclopropyl-N-((S)-6-(6-(6-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)-2,6-diazaspiro[3.3]heptane-2-carbonyl)pyridin-3-yl)-2,3-dihydrobenzofuran-3-yl)-N-methylquinoline- |
| | 6-carboxamide |
| A194 | 4-amino-N-((S)-6-(6-(4-((1-(2-((S)-1-((2S,4R)-1-((S)-2-(1-fluorocyclopropane-1-carboxamido)-3,3-dimethylbutanoyl)- |
| | 4-hydroxypyrrolidine-2-carboxamido)ethyl)-5-(4-methylthiazol-5-yl)phenyl)piperidin-4-yl)methyl)piperazine-1- |
| | carbonyl)pyridin-3-yl)-2,3-dihydrobenzofuran-3-yl)-N,1,3-trimethyl-1H-pyrazolo[4,3-c]quinoline-8-carboxamide |
| A196 | 4-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-N-methylpyrazolo[1,5-a]quinoxaline-8-carboxamide |
| A198 | 2-amino-3-cyclopropyl-N-((S)-6-(6-(4-((4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)cyclohexyl) |
| | methyl)piperazine-1-carbonyl)pyridin-3-yl)-2,3-dihydrobenzofuran-3-yl)-N-methylquinoline-6-carboxamide |
| A199 | 2-amino-3-cyano-7-fluoro-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A200 | 2-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-3-methoxy-N-methylquinoline-6-carboxamide |
| A201 | 2-amino-3-cyclopropyl-N-((S)-6-(6-(4-(1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidine-4- |
| | carbonyl)piperazine-1-carbonyl)pyridin-3-yl)-2,3-dihydrobenzofuran-3-yl)-N-methylquinoline-6-carboxamide |
| A202 | 4-amino-N-((S)-6-(6-(4-((1-(5-fluoro-2-((S)-1-((2S,4R)-1-((S)-2-(1-fluorocyclopropane-1-carboxamido)-3,3- |
| | dimethylbutanoyl)-4-hydroxypyrrolidine-2-carboxamido)ethyl)phenyl)piperidin-4-yl)methyl)piperazine-1- |
| | carbonyl)pyridin-3-yl)-2,3-dihydrobenzofuran-3-yl)-N,1-dimethyl-1H-pyrazolo[4,3-c]quinoline-8-carboxamide |
| A203 | 2-amino-3-cyclopropyl-N-((S)-6-(6-(4-(2-(1-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenyl)piperidin-4-yl)ethyl)piperazine-1-carbonyl)pyridin-3-yl)-2,3- |
| | dihydrobenzofuran-3-yl)-N-methylquinoline-6-carboxamide |
| A204 | 2-amino-3-cyclopropyl-N-((S)-6-(6-(4-((1-(2-(((2S,4R)-1-((S)-2- |
| | (1-fluorocyclopropane-1-carboxamido)-3,3-dimethylbutanoyl)-4-hydroxypyrrolidine-2- |
| | carboxamido)methyl)-5-(4-methylthiazol-5-yl)phenyl)-4-methylpiperidin-4-yl)methyl)piperazine- |
| | 1-carbonyl)pyridin-3-yl)-2,3-dihydrobenzofuran-3-yl)-N-methylquinoline-6-carboxamide |
| A206 | 2-amino-3-ethynyl-N-((S)-6-(6-(((1S,4R)-4-(2-(2-(((2S,4R)-1-((S)-2-(1-fluorocyclopropane-1- |
| | carboxamido)-3,3-dimethylbutanoyl)-4-hydroxypyrrolidine-2-carboxamido)methyl)-5-(4-methylthiazol-5- |
| | yl)phenoxy)ethoxy)cyclohexyl)carbamoyl)pyridin-3-yl)-2,3-dihydrobenzofuran-3-yl)-N-methylquinoline-6- |
| | carboxamide |
| A207 | 2-amino-3-cyano-N-((S)-6-(6-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethoxy)cyclohexyl)carbamoyl)pyridin-3-yl)-2,3-dihydrobenzofuran- |
| | 3-yl)-N-methylquinoline-6-carboxamide |
| A208 | 2-amino-N-((3S)-6-(6-(5-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl) |
| | piperidin-4-yl)methyl)octahydropyrrolo[3,4-c]pyrrole-2-carbonyl)pyridin-3-yl)-2,3-dihydrobenzofuran- |
| | 3-yl)-N-methyl-3-(oxetan-3-yl)quinoline-6-carboxamide |
| A209 | 2-amino-3-cyclopropyl-7-fluoro-N-((S)-6-(6-(4-((1-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenyl)piperidin- |
| | 4-yl)methyl)piperazine-1-carbonyl)pyridin-3-yl)-2,3-dihydrobenzofuran-3-yl)-N-methylquinoline-6-carboxamide |
| A210 | 2-amino-3-cyclopropyl-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)azetidin-3- |
| | yl)methyl)piperazine-1-carbonyl)pyridin-3-yl)-2,3-dihydrobenzofuran-3-yl)-N-methylquinoline-6-carboxamide |
| A211 | 2-amino-3-cyano-N-((S)-6-(6-((4-(2-(2-(((2S,4S)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)butyl) |
| | carbamoyl)pyridin-3-yl)-2,3-dihydrobenzofuran-3-yl)-N-methylquinoline-6-carboxamide |
| A213 | 2-amino-3-cyano-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)cyclohexyl)carbamoyl)pyridin- |
| | 3-yl)-2,3-dihydrobenzofuran-3-yl)-N-methylquinoline-6-carboxamide |
| A214 | 2-amino-3-cyano-N-((3S)-6-(6-(5-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)octahydropyrrolo[3,4-c]pyrrole-2-carbonyl)pyridin-3-yl)-2,3-dihydrobenzofuran-3-yl)-N- |
| | methylquinoline-6-carboxamide |
| A215 | 2-amino-3-cyano-N-((S)-6-(6-((4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | phenyl)carbamoyl)pyridin-3-yl)-2,3-dihydrobenzofuran-3-yl)-N-methylquinoline-6-carboxamide |
| A216 | 2-amino-3-cyano-N-((S)-6-(6-((6-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | spiro[3.3]heptan-2-yl)carbamoyl)pyridin-3-yl)-2,3-dihydrobenzofuran-3-yl)-N-methylquinoline-6-carboxamide |
| A217 | N-((S)-6-(6-(((1S,4S)-4-(2-(2-(((2S,4R)-1-((S)-2-acetamido-3,3-dimethylbutanoyl)-4-hydroxypyrrolidine-2- |
| | carboxamido)methyl)-5-fluorophenoxy)ethoxy)cyclohexyl)carbamoyl)pyridin-3-yl)-2,3-dihydrobenzofuran-3- |
| | yl)-2-amino-2-cyano-N-methylquinoline-6-carboxamide |
| A218 | 2-amino-3-cyano-N-((S)-6-(6-(4-((1-(2-(((2S,4S)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin- |
| | 4-yl)methyl)piperazine-1-carbonyl)pyridin-3-yl)-2,3-dihydrobenzofuran-3-yl)-N-methylquinoline-6-carboxamide |
| A219 | 2-amino-3-cyano-N-((S)-6-(6-(((1S,4S)-4-(2-(5-fluoro-2-(((2S,4R)-1-((S)-2-(1-fluorocyclopropane-1-carboxamido)- |
| | 3,3-dimethylbutanoyl)-4-hydroxypyrrolidine-2-carboxamido)methyl)phenoxy)ethoxy)cyclohexyl)carbamoyl) |
| | pyridin-3-yl)-2,3-dihydrobenzofuran-3-yl)-N-methylquinoline-6-carboxamide |
| A220 | 2-amino-3-cyano-N-((S)-6-(6-(((1S,3S)-3-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)cyclobutyl) |
| | carbamoyl)pyridin-3-yl)-2,3-dihydrobenzofuran-3-yl)-N-methylquinoline-6-carboxamide |
| A221 | 2-amino-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine- |
| | 1-carbonyl)pyridin-3-yl)-2,3-dihydrobenzofuran-3-yl)-3-isopropoxy-N-methylquinoline-6-carboxamide |
| A222 | 2-amino-3-cyano-N-((3S)-6-(6-((2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclobutyl)carbamoyl)pyridin-3-yl)-2,3-dihydrobenzofuran-3-yl)-N-methylquinoline-6-carboxamide |
| A223 | 2-amino-3-cyano-N-((S)-6-(6-((3-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | propyl)carbamoyl)pyridin-3-yl)-2,3-dihydrobenzofuran-3-yl)-N-methylquinoline-6-carboxamide |
| A224 | 2-amino-3-cyano-N-((3S)-6-(6-((3-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclopentyl)carbamoyl)pyridin-3-yl)-2,3-dimethylquinoline-6-carboxamide |
| A225 | 2-amino-3-cyano-N-((3S)-6-(6-((1-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethyl) |
| | piperidin-3-yl)carbamoyl)pyridin-3-yl)-2,3-dihydrobenzofuran-3-yl)-N-methylquinoline-6-carboxamide |
| A226 | 2-amino-3-cyano-N-((S)-6-(6-((2-((2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | methyl)benzyl)carbamoyl)pyridin-3-yl)-2,3-dihydrobenzofuran-3-yl)-N-methylquinoline-6-carboxamide |
| A227 | 2-amino-3-cyano-N-((S)-6-(6-(((3-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclobutyl)methyl)carbamoyl)pyridin-3-yl)-2,3-dihydrobenzofuran-3-yl)-N-methylquinoline-6-carboxamide |
| A228 | 2-amino-3-cyano-N-((S)-6-(6-(((1S,4S)-4-((3-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)prop-2-yn- |
| | 1-yl)oxy)cyclohexyl)carbamoyl)pyridin-3-yl)-2,3-dihydrobenzofuran-3-yl)-N-methylquinoline-6-carboxamide |
| A229 | 2-amino-3-cyclopropyl-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)-1,2,3,6- |
| | tetrahydropyridin-4-yl)methyl)piperazine-1-carbonyl)pyridin-3-yl)-2,3-dihydrobenzofuran-3-yl)-N- |
| | methylquinoline-6-carboxamide |
| A230 | 2-amino-3-cyano-N-((3S)-6-(6-((2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclopentyl)carbamoyl)pyridin-3-yl)-2,3-dihydrobenzofuran-3-yl)-N-methylquinoline-6-carboxamide |
| A231 | 2-amino-3-cyano-N-((S)-6-(6-((5-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)- |
| | 2-methylpentan-2-yl)carbamoyl)pyridin-3-yl)-2,3-dihydrobenzofuran-3-yl)-N-methylquinoline-6-carboxamide |
| A233 | 2-amino-3-cyano-N-((S)-6-(6-(((1S,2R)-2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methylquinoline-6-carboxamide |
| A234 | 2-amino-3-cyano-N-((S)-6-(6-((3-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | benzyl)carbamoyl)pyridin-3-yl)-2,3-dihydrobenzofuran-3-yl)-N-methylquinoline-6-carboxamide |
| A235 | 2-amino-3-cyano-N-((3S)-6-(6-(3-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin- |
| | 4-yl)methyl)-3,8-diazabicyclo[3.2.1]octane-8-carbonyl)pyridin-3-yl)-2,3-dihydrobenzofuran-3-yl)-N- |
| | methylquinoline-6-carboxamide |
| A236 | 2-amino-3-cyano-N-((S)-6-(6-(((1S,2R)-2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methylquinoline-6-carboxamide |
| A237 | 2-amino-3-cyano-N-((3S)-6-(6-(5-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl) |
| | piperidin-4-yl)methyl)-2,5-diazabicyclo[2.2.1]heptane-2-carbonyl)pyridin-3-yl)-2,3-dihydrobenzofuran- |
| | 3-yl)-N-methylquinoline-6-carboxamide |
| A239 | 2-amino-3-cyano-N-((3S)-6-(6-(((1-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethyl) |
| | azetidin-2-yl)methyl)carbamoyl)pyridin-3-yl)-2,3-dihydrobenzofuran-3-yl)-N-methylquinoline-6- |
| | carboxamide |
| A240 | 2-amino-3-cyano-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)-4- |
| | methylcyclohexyl)carbamoyl)pyridin-3-yl)-2,3-dihydrobenzofuran-3-yl)-N-methylquinoline-6-carboxamide |
| A241 | 2-amino-3-cyano-N-((S)-6-(6-(4-((4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl) |
| | piperidin-1-yl)methyl)piperidine-1-carbonyl)pyridin-3-yl)-2,3-dihydrobenzofuran-3-yl)-N-methylquinoline-6- |
| | carboxamide |
| A242 | 3-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N,1-dimethyl-1H-pyrazolo[4,3-c]quinoline-8- |
| | carboxamide |
| A243 | 2-amino-3-cyano-N-((S)-6-(6-(((1S,4S)-4-((4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)but-3-yn-1- |
| | yl)oxy)cyclohexyl)carbamoyl)pyridin-3-yl)-2,3-dihydrobenzofuran-3-yl)-N-methylquinoline-6-carboxamide |
| A244 | 2-amino-3-cyano-N-((S)-6-(6-((4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | but-2-yn-1-yl)carbamoyl)pyridin-3-yl)-2,3-dihydrobenzofuran-3-yl)-N-methylquinoline-6-carboxamide |
| A245 | 2-amino-3-cyano-N-((S)-6-(6-(((E)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | but-2-en-1-yl)carbamoyl)pyridin-3-yl)-2,3-dihydrobenzofuran-3-yl)-N-methylquinoline-6-carboxamide |
| A248 | 2-amino-3-(difluoromethyl)-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A249 | N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-N,3-dimethyl-2-(methylamino) |
| | quinoline-6-carboxamide |
| A250 | 4-amino-N-((S)-6-(6-(((1S,4S)-4-(2-(2-(((2S,4R)-1-((S)-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-N-methylpyrazolo[1,5-a] |
| | quinoxaline-8-carboxamide |
| A251 | 2-amino-3-cyano-N-((S)-6-(6-(((1S,4S)-4-(5-((2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)methyl)- |
| | 1H-pyrazol-1-yl)cyclohexyl)carbamoyl)pyridin-3-yl)-2,3-dihydrobenzofuran-3-yl)-N-methylquinoline-6-carboxamide |
| A252 | 2-amino-3-cyano-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-isopropylquinoline-6-carboxamide |
| A254 | 2-amino-3-ethynyl-N-((S)-6-(6-(4-((4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-1- |
| | yl)methyl)piperidine-1-carbonyl)pyridin-3-yl)-2,3-dihydrobenzofuran-3-yl)-N-methylquinoline-6-carboxamide |
| A255 | 2-amino-3-(difluoromethyl)-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methylquinoline-6-carboxamide |
| A256 | 4-amino-3-cyclopropyl-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methyl-1H-pyrazolo[4,3-c]quinoline- |
| | 8-carboxamide |
| A257 | 2-amino-3-ethynyl-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)-5-methylpyridin-3-yl)-2,3-dihydrobenzofuran-3-yl)-N-methylquinoline-6-carboxamide |
| A259 | 2-amino-3-ethynyl-N-((S)-6-(5-fluoro-6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methylquinoline-6-carboxamide |
| A260 | N1-(3-amino-1-methyl-1H-pyrazolo[4,3-c]pyridin-7-yl)-N2-((S)-6-(6-(4-((1-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenyl)piperidin-4-yl)methyl)piperazine-1-carbonyl)pyridin-3-yl)-2,3- |
| | dihydrobenzofuran-3-yl)-N2-methyloxalamide |
| A262 | 2-amino-3-ethynyl-N-((S)-6-(3-fluoro-4-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethoxy)cyclohexyl)carbamoyl)phenyl)-2,3-dihydrobenzofuran- |
| | 3-yl)-N-methylquinoline-6-carboxamide |
| A263 | 2-amino-3-cyano-N-((3S)-6-(6-(((1S,4S)-4-(2-((2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenoxy)methyl)pyrrolidin-1-yl)cyclohexyl)carbamoyl)pyridin-3-yl)- |
| | 2,3-dihydrobenzofuran-3-yl)-N-methylquinoline-6-carboxamide |
| A264 | 2-amino-3-cyano-N-((S)-6-(6-(((S)-1-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethyl)- |
| | 4,5,6,7-tetrahydro-1H-indazol-5-yl)carbamoyl)pyridin-3-yl)-2,3-dihydrobenzofuran-3-yl)-N-methylquinoline- |
| | 6-carboxamide |
| A265 | 2-amino-3-ethynyl-N-((S)-6-(4-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)phenyl)-2,3-dihydrobenzofuran-3-yl)-N-methylquinoline-6-carboxamide |
| A266 | 2-amino-3-cyano-N-((S)-6-(6-(((1S,4S)-4-(2-(2-(((2R,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)-1- |
| | methylcyclohexyl)carbamoyl)pyridin-3-yl)-2,3-dihydrobenzofuran-3-yl)-N-methylquinoline-6-carboxamide |
| A267 | 2-amino-3-ethynyl-N-((S)-6-(6-(4-(1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidine- |
| | 4-carbonyl)piperazine-1-carbonyl)pyridin-3-yl)-2,3-dihydrobenzofuran-3-yl)-N-methylquinoline-6-carboxamide |
| A268 | 2-amino-3-ethynyl-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-isopropylquinoline-6-carboxamide |
| A269 | 2-amino-3-cyano-N-((S)-6-(6-(((4-((2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | methyl)-2,5-dihydrofuran-3-yl)methyl)carbamoyl)pyridin-3-yl)-2,3-dihydrobenzofuran-3-yl)-N- |
| | methylquinoline-6-carboxamide |
| A270 | 2-amino-3-ethynyl-N-((3S)-6-(6-(3-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)-3,8-diazabicyclo[3.2.1]octane-8-carbonyl)pyridin-3-yl)-2,3-dihydrobenzofuran-3-yl)-N- |
| | methylquinoline-6-carboxamide |
| A271 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-N-methyl-3-(1,1,1- |
| | trifluoropropan-2-yl)quinoline-6-carboxamide |
| A272 | N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-N-methyl-4- |
| | (methylamino)-1,3-dihydrofuro[3,4-c][1,7]naphthyridine-8-carboxamide |
| A274 | 2-amino-3-cyano-N-((S)-6-(6-((6-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)pyridin-3-yl)carbamoyl)pyridin-3-yl)-2,3-dihydrobenzofuran-3-yl)-N-methylquinoline-6-carboxamide |
| A275 | 2-amino-3-cyano-N-((S)-6-(6-(((1S,4S)-4-(4-((2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)methyl)- |
| | 1H-1,2,3-triazol-1-yl)cyclohexyl)carbamoyl)pyridin-3-yl)-2,3-dihydrobenzofuran-3-yl)-N-methylquinoline-6- |
| | carboxamide |
| A276 | 2-amino-3-cyano-N-((S)-6-(6-(((1S,4S)-4-(5-((2-(((2S,4R)-1-((S)-2-(1-fluorocyclopropane-1-carboxamido)-3,3- |
| | dimethylbutanoyl)-4-hdyroxypyrrolidine-2-carboxamido)methyl)-5-(4-methylthiazol-5-yl)phenoxy)methyl)-1H- |
| | 1,2,3-triazol-1-yl)cyclohexyl)carbamoyl)pyridin-3-yl)-2,3-dihydrobenzofuran-3-yl)-N-methylquinoline-6-carboxamide |
| A278 | N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-N,1-dimethyl-3-(methylamino)- |
| | 1H-pyrazolo[4,3-c]quinoline-8-carboxamide |
| A279 | 2-amino-3-cyano-N-((3S)-6-(6-(((1R,3R,5S)-6-((2-(2-(((2S,4R)-1-((S)-2- |
| | (1-fluorocyclopropane-1-carboxamido)-3,3-dimethylbutanoyl)-4-hydroxypyrrolidine-2-carboxamido)methyl)- |
| | 5-(4-methylthiazol-5-yl)phenoxy)ethyl)amino)bicyclo[3.1.0]hexan-3-yl)carbamoyl)pyridin-3-yl)-2,3- |
| | dihydrobenzofuran-3-yl)-N-methylquinoline-6-carboxamide |
| A280 | 2-amino-3-cyano-N-((3S)-6-(6-(((1R,3S,5S)-6-((2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethyl) |
| | amino)bicyclo[3.1.0]hexan-3-yl)carbamoyl)pyridin-3-yl)-2,3-dihydrobenzofuran-3-yl)-N-methylquinoline-6- |
| | carboxamide |
| A281 | N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-2-(isopropylamino)-N,3- |
| | dimethylquinoline-6-carboxamide |
| A282 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-3-methoxy-N-methylquinoline-6- |
| | carboxamide |
| A283 | 2-amino-3-ethynyl-N-((S)-6-(6-((1-(3-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | propyl)piperidin-4-yl)carbamoyl)pyridin-3-yl)-2,3-dihydrobenzofuran-3-yl)-N-methylquinoline-6-carboxamide |
| A284 | 2-amino-3-ethynyl-N-((S)-6-(6-((3-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | benzyl)carbamoyl)pyridin-3--yl)-2,3-dihydrobenzofuran-3-yl)-N-methylquinoline-6-carboxamide |
| A285 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-3-(1-fluorocyclopropyl)-N- |
| | methylquinoline-6-carboxamide |
| A286 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methyl-3-(3,3,3-trifluoroprop-1- |
| | en-2-yl)quinoline-6-carboxamide |
| A287 | 2-amino-3-ethynyl-N-((S)-6-(2-((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)-1-oxoisoindolin-5-yl)-2,3-dihydrobenzofuran-3-yl)-N-methylquinoline-6-carboxamide |
| A288 | 2-amino-3-ethynyl-N-((S)-6-(6-((1′-(2-(((2S,4R)-1-((S)-2-(1-fluorocyclopropane-1-carboxamido)-3,3- |
| | methylbutanoyl)-4-hydroxypyrrolidine-2-carboxamido)methyl)-5-(4-methylthiazol-5-yl)phenyl)-[1,4′- |
| | bipiperidin]-4-yl)carbamoyl)pyridin-3-yl)-2,3-dihydrobenzofuran-3-yl)-N-methylquinoline-6-carboxamide |
| A289 | N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N,1-dimethyl-1H-pyrazolo[4,3-c] |
| | quinoline-8-carboxamide |
| A290 | 4-amino-N-((S)-6-(6-(9-(2-(2-(2-(2-(((2S,4S)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)ethoxy)ethyl)-3,9-diazaspiro[5.5]undecane-3-carbonyl)pyridin-3-yl)-2,3-dihydrobenzofuran- |
| | 3-yl)-N,1-dimethyl-1H-pyrazolo[4,3-c]quinoline-8-carboxamide |
| A291 | 2-amino-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl) |
| | piperidin-4-yl)methyl)piperazine-1-carbonyl)pyridin-3-yl)-2,3-dihydrobenzofuran-3-yl)-N-methyl-3- |
| | (1-methylcyclopropyl)quinoline-6-carboxamide |
| A292 | N6-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N6-methyl-2-(methylamino)quinoline- |
| | 3,6-dicarboxamide |
| A293 | 3-cyano-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-N-methyl-2-(methylamino) |
| | quinoline-6-carboxamide |
| A294 | 2-amino-3-ethynyl-N-((3S)-6-(6-(5-(1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)octahydropyrrolo[3,4-c]pyrrole-2-carbonyl)pyridin-3-yl)-2,3-dihydrobenzofuran-3-yl)-N- |
| | methylquinoline-6-carboxamide |
| A295 | 2-amino-3-ethynyl-N-((3S)-6-(6-(8-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)-3,8-diazabicyclo[3.2.1]octane-3-carbonyl)pyridin-3-yl)-2,3-dihydrobenzofuran-3-yl)-N- |
| | methylquinoline-6-carboxamide |
| A297 | 3-ethynyl-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methyl-2-(methylamino)quinoline-6-carboxamide |
| A298 | 2-amino-3-cyano-N-((S)-6-(6-((2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethyl)-2-azaspiro[4.5]decan-8-yl)carbamoyl)pyridin-3-yl)-2,3-dihydrobenzofuran-3-yl)-N-methylquinoline-6-carboxamide |
| A299 | 2-amino-3-ethynyl-N-((S)-6-(6-((1-(1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)azetidin-3- |
| | yl)piperidin-4-yl)carbamoyl)pyridin-3-yl)-2,3-dihydrobenzofuran-3-yl)-N-methylquinoline-6-carboxamide |
| A301 | 2-amino-3-ethynyl-N-((3S)-6-(6-(3-(2′-(((2S,4R)-1-((S)-2-(1-fluorocyclopropane-1-carboxamido)-3,3- |
| | dimethylbutanoyl)-4-hydroxypyrrolidine-2-carboxamido)methyl)-5′-(4-methylthiazol-5-yl)-2,3,4,5- |
| | tetrahydro-[1,1′-biphenyl]-4-yl)-3,8-diazabicyclo[3.2.1]octane-8-carbonyl)pyridin-3-yl)-2,3-dihydrobenzofuran- |
| | 3-yl)-N-methylquinoline-6-carboxamide |
| A303 | 2-amino-3-ethynyl-N-((S)-6-(6-(((1S,4S)-4-(4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperazin-1- |
| | yl)cyclohexyl)carbamoyl)pyridin-3-yl)-2,3-dihydrobenzofuran-3-yl)-N-methylquinoline-6-carboxamide |
| A304 | 2-amino-3-ethynyl-N-((S)-6-(5-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyrazin-2-yl)-2,3-dihydrobenzofuran-3-yl)-N-methylquinoline-6-carboxamide |
| A305 | 2-amino-3-(3,3-difluorocyclobutyl)-N-((S)-6-(6-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethoxy)cyclohexyl)carbamoyl)pyridin-3-yl)-2,3- |
| | dihydrobenzofuran-3-yl)-N-methylquinoline-6-carboxamide |
| A306 | 2-amino-3-ethynyl-N-((S)-6-(5-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethoxy)cyclohexyl)carbamoyl)yl)pyridin-2-yl)-2,3- |
| | dihydrobenzofuran-3-yl)-N-methylquinoline-6-carboxamide |
| A307 | 2-amino-3-ethynyl-N-((3S)-6-(6-(3-(1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin- |
| | 4-yl)-3,8-diazabicyclo[3.2.1]octane-8-carbonyl)pyridin-3-yl)-2,3-dihydrobenzofuran-3-yl)-N- |
| | methylquinoline-6-carboxamide |
| A308 | 2-amino-3-ethynyl-N-((3S)-6-(6-(3-((1S,4r)-4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)cyclohexyl)- |
| | 3,8-diazabicyclo[3.2.1]octane-8-carbonyl)pyridin-3-yl)-2,3-dihydrobenzofuran-3-yl)-N-methylquinoline-6-carboxamide |
| A309 | 2-amino-3-ethynyl-N-((3S)-6-(6-(((3S,6R)-6-((2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | methyl)bicyclo[3.1.0]hexan-3-yl)carbamoyl)pyridin-3-yl)-2,3-dihydrobenzofuran-3-yl)-N-methylquinoline-6-carboxamide |
| A310 | 2-amino-3-ethynyl-N-((3S)-6-(6-(((3R,6S)-6-((2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | methyl)bicyclo[3.1.0]hexan-3-yl)carbamoyl)pyridin-3-yl)-2,3-dihydrobenzofuran-3-yl)-N-methylquinoline-6-carboxamide |
| A311 | 2-amino-3-ethynyl-N-((3S)-6-(6-(3-((1R,4s)-4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)cyclohexyl)- |
| | 3,8-diazabicyclo[3.2.1]octane-8-carbonyl)pyridin-3-yl)-2,3-dihydrobenzofuran-3-yl)-N-methylquinoline-6-carboxamide |
| A312 | 2-amino-3-ethynyl-N-((3S)-6-(6-(3-((4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)cyclohexyl) |
| | methyl)-3,8-diazabicyclo[3.2.1]octane-8-carbonyl)pyridin-3-yl)-2,3-dihydrobenzofuran-3-yl)-N- |
| | methylquinoline-6-carboxamide |
| A313 | 2-amino-3-ethynyl-N-((3S)-6-(6-(3-(4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)cyclohexyl)-3,8- |
| | diazabicyclo[3.2.1]octane-8-carbonyl)pyridin-3-yl)-2,3-dihydrobenzofuran-3-yl)-N-methylquinoline-6-carboxamide |
| A314 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)yl)pyridin-3-yl)-2,3-dihydrobenzofuran-3-yl)-3-(2-hydroxyethyl)-N-methylquinoline-6-carboxamide |
| A315 | N1-(7-amino-2-ethyl-2H-pyrazolo[3,4-c]pyridin-4-yl)-N2-((S)-6-(6-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2-carboxamido)methyl)- |
| | 5-(4-methylthiazol-5-yl)phenoxy)ethoxy)cyclohexyl)carbamoyl)pyridin-3-yl)-2,3-dihydrobenzofuran-3-yl)- |
| | N2-methyloxalamide |
| A316 | 2-amino-3-ethynyl-N-((S)-6-(2-((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)-3-oxoisoindolin-5-yl)-2,3-dihydrobenzofuran-3-yl)-N-methylquinoline-6-carboxamide |
| A318 | 2-amino-3-ethynyl-N-((3S)-6-(6-(3-((2′-(((2S,4R)-1-((S)-2-(1-fluorocyclopropane-1-carboxamido)-3,3- |
| | dimethylbutanoyl)-4-hydroxypyrrolidine-2-carboxamido)methyl)-5′-(4-methylthiazol-5-yl)-2,3,4,5-tetrahydro- |
| | [1,1′-biphenyl]-4-yl)methyl)-3,8-diazabicyclo[3.2.1]octane-8-carbonyl)pyridin-3-yl)-2,3-dihydrobenzofuran-3- |
| | yl)-N-methylquinoline-6-carboxamide |
| A319 | N1-(6-amino-4-(3-hydroxypropyl)pyridin-3-yl)-N2-((S)-6-(6-(((1S,4S)-4-(2-(2-(((2S,4R)-1-((S)-2- |
| | (1-fluorocyclopropane-1-carboxamido)-3,3-dimethylbutanoyl)-4-3,3-dimethylbutanoyl)-4-hydroxypyrrolidine-2- |
| | carboxamido)methyl)-5-(4-methylthiazol-5-yl)phenoxy)ethoxy)cyclohexyl)carbamoyl)pyridin-3-yl)-2,3-dihydro- |
| | benzofuran-3-yl)-N2-methyloxalamide |
| A321 | 2-amino-3-(azetidin-1-yl)-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A323 | 2-amino-3-(tert-butyl)-N-((S)-6-(6-(4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A324 | N-((S)-6-(2-((1S,4S)-4-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)cyclohexyl)-1-oxoisoindolin- |
| | 5-yl)-2,3-dihydrobenzofuran-3-yl)-N,1,3-trimethyl-1H-pyrazolo[4,3-c]quinoline-8-carboxamide |
| A325 | N1-((S)-6-(6-(((1S,4S)-4-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)cyclohexyl)carbamoyl) |
| | pyridin-3-yl)-2,3-dihydrobenzofuran-3-yl)-N1-methyl-N2-(1-methyl-1H-pyrazolo[4,3-c]pyridin-7-yl)oxalamide |
| A326 | N-((S)-6-(6-(((1S,4S)-4-(2-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)cyclohexyl)carbamoyl) |
| | pyridin-3-yl)-2,3-dihydrobenzofuran-3-yl)-N,1,3-trimethyl-1H-pyrazolo[4,3-c]quinoline-8-carboxamide |
| A327 | N1-(4-amino-2-ethyl-2,3-dihydro-1H-pyrrolo[3,4-c]pyridin-7-yl)-N2-((S)-6-(6-(((1S,4S)-4-(2-(2-(((2S,4R)- |
| | 1-((S)-2-(1-fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethoxy)cyclohexyl)carbamoyl)pyridin-3-yl)-2,3-dihydrobenzofuran- |
| | 3-yl)-N2-methyloxalamide |
| A328 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-4-(3-hydroxypropyl)-N-methylquinoline- |
| | 6-carboxamide |
| A329 | 3-ethyl-N-((S)-6-(2-((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)-1-oxoisoindolin-5-yl)-2,3-dihydrobenzofuran-3-yl)-N,1-dimethyl-1H-pyrazolo |
| | [4,3-c]quinoline-8-carboxamide |
| A330 | 3-ethyl-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N,1-dimethyl-1H-pyrazolo[4,3-c] |
| | quinoline-8-carboxamide |
| A331 | 3-cyclopropyl-N-((S)-6-(6-(((1S,4S)-4-(2-(2-(((2S,4R)-1-((S)-2-(1-fluorocyclopropane-1-carboxamido)-3,3- |
| | hydroxypyrrolidine-2-carboxamido)methyl)-5-(4-methylthiazol-5-yl)phenoxy)ethoxy)cyclohexyl)carbamoyl) |
| | pyridin-3-yl)-2,3-dihydrobenzofuran-3-yl)-N,1-dimethyl-1H-pyrazolo[4,3-c]quinoline-8-carboxamide |
| A334 | 3-cyclopropyl-N-((S)-6-(6-(4-((1-(2-(((2S,4R)-1-((S)-2-(1-fluorocyclopropane-1-carboxamido)-3,3-dimethylbutanoyl)- |
| | 4-hydroxypyrrolidine-2-carboxamido)methyl)-5-(4-methylthiazol-5-yl)phenyl)piperidin-4-yl)methyl)piperazine-1- |
| | carbonyl)pyridin-3-yl)-2,3-dihydrobenzofuran-3-yl)-N,1-dimethyl-1H-pyrazolo[4,3-c]quinoline-8-carboxamide |
| A335 | 2-amino-3-ethynyl-N-((S)-6-(6-(((1S,3R,4S)-3-fluoro-4-(2-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenoxy)ethoxy)cyclohexyl)carbamoyl)pyridin-3-yl)-2,3-dihydrobenzofuran- |
| | 3-yl)-N-methylquinoline-6-carboxamide |
| A336 | 2-amino-3-ethynyl-N-((3S)-6-(6-(3-(3-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)cyclobutyl)-3,8- |
| | diazabicyclo[3.2.1]octane-8-carbonyl)pyridin-2-yl)-2,3-dihydrobenzofuran-3-yl)-N-methylquinoline-6-carboxamide |
| A337 | 2-amino-3-ethynyl-N-((3S)-6-(6-(3-(4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)phenyl)- |
| | 3,8-diazabicyclo[3.2.1]octane-8-carbonyl)pyridin-3-yl)-2,3-dihydrobenzofuran-3-yl)-N-methylquinoline-6-carboxamide |
| A338 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)cyclohexyl) |
| | carbamoyl)pyridin-3-yl)-2,3-dihydrobenzofuran-3-yl)-3-(4-hydroxybutyl)-N-methylquinoline-6-carboxamide |
| A339 | 2-amino-3-ethynyl-N-((S)-6-(2-((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)cyclohexyl)- |
| | 1-oxo-1,2,3,4-tetrahydroisoquinolin-6-yl)-2,3-dihydrobenzofuran-3-yl)-N-methylquinoline-6-carboxamide |
| A341 | 2-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-3-(2-hydroxyethoxy)-N-methylquinoline-6- |
| | carboxamide |
| A342 | 2-amino-3-ethynyl-N-((3S)-6-(6-(3-((2′-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5′-yl)-1,2,3,6-tetrahydro- |
| | [1,1′-biphenyl]-4-yl)methyl-3,8-diazabicyclo[3.2.1]octane-8-carbonyl)pyridin-3-yl)-2,3-dihydrobenzofuran-3- |
| | yl)-N-methylquinoline-6-carboxamide |
| A343 | 2-amino-3-ethynyl-N-((S)-6-(6-(((1S,4S)-4-(3-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)propoxy) |
| | cyclohexyl)carbamoyl)pyridin-3-yl)-2,3-dihydrobenzofuran-3-yl)-N-methylquinoline-6-carboxamide |
| A344 | 2-amino-3-ethynyl-N-((S)-6-(3-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)amino)-1H-indazol-6-yl)-2,3-dihydrobenzofuran-3-yl)-N-methylquinoline-6-carboxamide |
| A345 | 4-amino-N-((S)-6-(6-(((1S,4S)-4-(2-(2-(((2S,4S)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-N,1-dimethyl-1H-pyrazolo |
| | [4,3-c]quinoline-8-carboxamide |
| A346 | 2-amino-3-ethynyl-N-((S)-6-(6-(((1S,4S)-4-((2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)-2- |
| | methylpropyl)(methyl)amino)cyclohexyl)carbamoyl)pyridin-3-yl)-2,3-dihydrobenzofuran-3-yl)-N- |
| | methylquinoline-6-carboxamide |
| A347 | 2-amino-3-ethynyl-N-((S)-6-(6-(((1S,4S)-4-(2-((2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)amino)-2- |
| | oxoethoxy)cyclohexyl)carbamoyl)pyridin-3-yl)-2,3-dihydrobenzofuran-3-yl)-N-methylquinoline-6-carboxamide |
| A350 | 2-amino-3-ethynyl-N-((S)-6-(6-(((1S,4S)-4-((1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)-2- |
| | methylpropan-2-yl)oxy)cyclohexyl)carbamoyl)pyridin-3-yl)-2,3-dihydrobenzofuran-3-yl)-N-methylquinoline-6- |
| | carboxamide |
| A352 | 2-amino-3-ethynyl-N-((S)-6-(6-(((1S,4S)-4-(2-(2-((S)-1-((2S,4R)-1-((S)-2-(1-fluorocyclopropane-1-carboxamido)-3,3- |
| | dimethylbutanoyl)-4-hydroxypyrrolidine-2-carboxamido)ethyl)-5-(4-methylthiazol-5-yl)phenoxy)ethoxy)cyclohexyl) |
| | carbamoyl)yl)pyridin-3-yl)-2,3-dihydrobenzofuran-3-yl)-N-methylquinoline-6-carboxamide |
| A353 | 2-amino-N-((S)-6-(6-(((1S,4S)-4-(2-(2-(((2S,4R)-1-((S)-2-(1-cyanocyclopropane-1-carboxamido)-3,3-dimethylbutanoyl)- |
| | 4-hydroxypyrrolidine-2-carboxamido)methyl)-5-(4-methylthiazol-5-yl)phenoxy)ethoxy)cyclohexyl)carbamoyl) |
| | pyridin-3-yl)-2,3-dihydrofuran-3-yl)-3-ethynyl-N-methylquinoline-6-carboxamide |
| A354 | 2-amino-3-ethynyl-N-((S)-6-(6-((((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)oxy)methyl)pyridin-3-yl)-2,3-dihydrobenzofuran-3-yl)-N-methylquinoline-6-carboxamide |
| A355 | 2-amino-N-((S)-6-(6-(((1S,4S)-4-(2-((4′-(cyanomethyl)-4-(((2S,4R)-1-((S)-2-(1-fluorocyclopropane-1-carboxamido)-3,3- |
| | dimethylbutanoyl)-4-hydroxypyrrolidine-2-carboxamido)methyl)-[1,1′-biphenyl]-3-yl)oxy)ethoxy) |
| | cyclohexyl)carbamoyl)pyridin-3-yl)-2,3-dihydrobenzofuran-3-yl)-3-ethynyl-N-methylquinoline-6-carboxamide |
| A357 | 2-amino-3-ethynyl-N-((S)-6-(6-(((1R,4R)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)cyclohexyl) |
| | carbamoyl)pyridin-3-yl)-2,3-dihydrobenzofuran-3-yl)-N-methylquinoline-6-carboxamide |
| A358 | 2-amino-N-((S)-6-(6-(((1S,4S)-4-(2-((2′,4′-dichloro-4-(((2S,4R)-1-((S)-2-(1-fluorocyclopropane-1- |
| | carboxamido)-3,3-dimethylbutanoyl)-4-hydroxypyrrolidine-2-carboxamido)methyl)-[1,1′-biphenyl]-3- |
| | yl)oxy)ethoxy)cyclohexyl)carbamoyl)pyridin-3-yl)-2,3-dihydrobenzofuran-3-yl)-3-ethynyl-N- |
| | methylquinoline-6-carboxamide |
| A359 | 2-amino-N-((S)-6-(6-(((1S,4S)-4-(2-(2-(((2S,4R)-1-((S)-2-(bicyclo[1.1.1]pentane-1-carboxamido)- |
| | 3,3-dimethybutanoyl)-4-hydroxypyrrolidine-2-carboxamido)methyl)-5-(4-methylthiazol-5-yl)phenoxy) |
| | ethoxy)cyclohexyl)carbamoyl)pyridin-3-yl)-2,3-dihydrobenzofuran-3-yl)-3-ethynyl-N-methylquinoline-6- |
| | carboxamide |
| A360 | N-((3S)-6-(6-(((1S,4S)-4-(2-(2-(((2S,4R)-1-((2S)-2-((1R,3R,5S)-adamantante-1-carboxamido)-3,3-dimethylbutanoyl)- |
| | 4-hydroxypyrrolidine-2-carboxamido)methyl)-5-(4-methylthiazol-5-yl)phenoxy)ethoxy)cyclohexyl) |
| | carbamoyl)pyridin-3-yl)-2,3-dihydrobenzofuran-3-yl)-2-amino-3-ethynyl-N-methylquinoline-6-carboxamide |
| A363 | 2-amino-3-ethynyl-N-((S)-6-(6-(((1R,4S)-4-(2-(2-((R)-1-((2S,4R)-1-carboxamido)-3,3-dimethylbutanoyl)-4- |
| | hydroxypyrrolidine-2-carboxamido)-2-hydroxyethyl)-5-(4-methylthiazol-5-yl)phenoxy)ethoxy)cyclohexyl) |
| | carbamoyl)pyridin-3-yl)-2,3-dihydrobenzofuran-3-yl)-N-methylquinoline-6-carboxamide |
| A364 | 4-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N,3-dimethylimidazo[1,5-a]quinoline-8-carboxamide |
| A365 | 2-amino-N-((S)-6-(6-(((1S,4S)-4-(2-(2-(((2S,4R)-1-((S)-2-(1-difluoromethyl)cyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-3-ethynyl-N-methylquinoline-6-carboxamide |
| A366 | 4-amino-N-((S)-6-(6-(((1R,4R)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy)cyclohexyl) |
| | carbamoyl)pyridin-3-yl)-2,3-dihydrobenzofuran-3-yl)-N,1-dimethyl-1H-pyrazolo[4,3-c]quinoline-8-carboxamide |
| A367 | 2-amino-3-cyclopropyl-N-((S)-6-(6-(((1R,4R)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methylquinoline-6-carboxamide |
| A368 | 2-amino-3-ethynyl-N-((3S)-6-(6-(3-(4-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)piperidin-1- |
| | yl)-8-azabicyclo[3.2.1]octane-8-carbonyl)pyridin-3-yl)-2,3-dihydrobenzofuran-3-yl)-N-methylquinoline-6-carboxamide |
| A369 | 2-amino-3-cyano-N-((S)-6-(6-((4-(2-(2-(((2S,4R)-1-((S)-2-cyclopentyl-2-(1-fluorocyclopropane-1-carboxamido) |
| | acetyl)-4-hydroxypyrrolidine-2-carboxamido)methyl)-5-(4-methylthiazol-5-yl)phenoxy)ethoxy)cyclohexyl)carbamoyl) |
| | pyridin-3-yl)-2,3-dihydrobenzofuran-3-yl)-N-methylquinoline-6-carboxamide |
| A370 | 2-amino-3-ethynyl-N-((3S)-6-(6-(((1R,3S,5R)-5-(2-(2-(((2S,4R)-1-((S)-2- |
| | (1-fluorocyclopropane-1-carboxamido)-3,3-dimethylbutanoyl)-4-hydroxypyrrolidine-2- |
| | carboxamido)methyl)-5-(4-methylthiazol-5-yl)phenoxy)ethoxy)adamantan-2-yl)carbamoyl)pyridin- |
| | 3-yl)-2,3-dihydrobenzofuran-3-yl)-N-methylquinoline-6-carboxamide |
| A371 | 2-amino-3-cyano-N-((S)-6-(6-((4-(2-(2-(((2S,4R)-1-((S)-2-(1-fluorocyclopropane-1-carboxamido)- |
| | 2-phenylacetyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methylquinoline-6-carboxamide |
| A373 | 2-amino-3-cyclopropyl-N-((S)-6-(6-(4-((4-fluoro-1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A374 | 2-amino-N-((S)-6-(6-(((1S,4S)-4-(2-(5-cyano-2-(((2S,4R)-1-((S)-2-(1-fluorocyclopropane-1-carboxamido)-3,3- |
| | dimethylbutanoyl)-4-hydroxypyrrolidine-2-carboxamido)methyl)phenoxy)ethoxy)cyclohexyl)carbamoyl)pyridin-3- |
| | yl)-2,3-dihydrobenzofuran-3-yl)-3-cyclopropyl-N-methylquinoline-6-carboxamide |
| A375 | 2-amino-N-((S)-6-(6-(((1S,4S)-4-(2-((4′-cyano-4-(((2S,4R)-1-((S)-2-(1-fluorocyclopropane-1-carboxamido)-3,3- |
| | dimethylbutanoyl)-4-hydroxypyrrolidine-2-carboxamido)methyl)-[1,1′-biphenyl]-3-yl)oxy)ethoxy)cyclohexyl) |
| | carbamoyl)pyridin-3-yl)-2,3-dihydrobenzofuran-3-yl)-3-ethynyl-N-methylquinoline-6-carboxamide |
| A376 | 2-amino-3-ethynyl-N-((S)-6-(6-((4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | bicyclo[2.2.2]octan-1-yl)carbamoyl)pyridin-3-yl)-2,3-dihydrobenzofuran-3-yl)-N-methylquinoline-6-carboxamide |
| A378 | 2-amino-3-cyano-N-((S)-6-(6-(4-((4-fluoro-1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)methyl)piperazine-1-carbonyl)pyridin-3-yl)-2,3-dihydrobenzofuran-3-yl)-N-methylquinoline-6-carboxamide |
| A379 | N1-(6-amino-5-cyanopyridin-3-yl)-N2-((S)-6-(6-(((1S,4S)-4-(2-(2-(((2S,4R)-1-((S)-2-(1-fluorocyclopropane-1-carboxamido)- |
| | 3,3-dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N2-methyloxalamide |
| A380 | 2-amino-3-cyclopropyl-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(prop-1-yn-1-yl)phenoxy)ethoxy)cyclohexyl) |
| | carbamoyl)pyridin-3-yl)-2,3-dihydrobenzofuran-3-yl)-N-methylquinoline-6-carboxamide |
| A383 | 2-amino-3-ethynyl-N-((S)-6-(6-(4-(3-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)propyl) |
| | piperazine-1-carbonyl)pyridin-3-yl)-2,3-dihydrobenzofuran-3-yl)-N-methylquinoline-6-carboxamide |
| A384 | 2-amino-3-ethynyl-N-((S)-6-(6-(((1R,4S)-4-(2-(2-((R)-1-((2S,4R)-1-((S)-2- |
| | (1-fluorocyclopropane-1-carboxamido)-3,3-dimethylbutanoyl)-4-hydroxypyrrolidine-2-carboxamido)- |
| | 2-morpholinoethyl)-5-(4-methythiazol-5-yl)phenoxy)ethoxy)cyclohexyl)carbamoyl)pyridin-3-yl)-2,3- |
| | dihydrobenzofuran-3-yl)-N-methylquinoline-6-carboxamide |
| A385 | N1-(6-amino-5-cyclopropylpyridin-3-yl)-N2-((S)-6-(6-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2- |
| | carboxamido)methyl)-5-(4-methylthiazol-5-yl)phenoxy)ethoxy)cyclohexyl)carbamoyl) |
| | pyridin-3-yl)-2,3-dihydrobenzofuran-3-yl)-N2-methyloxalamide |
| A386 | 2-amino-3-ethynyl-N-((S)-6-(6-(((1S,4S)-4-(2-(((2S,4R)-1-((S)-2- |
| | (1-fluorocyclopropane-1-carboxamido)-3,3-dimethylbutanoyl)-4-hydroxypyrrolidine-2- |
| | carboxamido)methyl)-5-(4-methylthiazol-5-yl)phenethoxy)cyclohexyl)carbamoyl)pyridin-3-yl)- |
| | 2,3-dihydrobenzofuran-3-yl)-N-methylquinoline-6-carboxamide |
| A387 | N1-(6-amino-5-ethynylpyridin-3-yl)-N2-((S)-6-(6-(((1S,4S)-4-(2-(2-(((2S,4R)-1-((S)-2- |
| | (1-fluorocyclopropane-1-carboxamido)-3,3-dimethylbutanoyl)-4-hydroxypyrrolidine-2- |
| | carboxamido)methyl)-5-(4-methylthiazol-5-yl)phenoxy)ethoxy)cyclohexyl)carbamoyl)pyridin-3-yl)- |
| | 2,3-dihydrobenzofuran-3-yl)-N2-methyloxalamide |
| A389 | 2-amino-3-cyano-N-((S)-6-(6-((4-(2-(2-(((2S,4R)-1-((S)-2-cyclohexyl-2-(1-fluorocyclopropane-1- |
| | carboxamido)acetyl)-4-hydroxypyrrolidine-2-carboxamido)methyl)-5-(4-methylthiazol-5-yl) |
| | phenoxy)ethoxy)cyclohexyl)carbamoyl)pyridin-3-yl)-2,3-dihydrobenzofuran-3-yl)-N- |
| | methylquinoline-6-carboxamide |
| A390 | 2-amino-3-ethynyl-N-((S)-6-(6-(4-(2-(1-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenyl)piperidin-4- |
| | yl)acetyl)piperazine-1-carbonyl)pyridin-3-yl)-2,3-dihydrobenzofuran-3-yl)-N-methylquinoline-6-carboxamide |
| A391 | 4-amino-N-((S)-6-(4-((4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy) |
| | ethoxy)bicyclo[2.2.2]octan-1-yl)carbamoyl)phenyl)-2,3-dihydrobenzofuran-3-yl)-N-methyl-1,3- |
| | dihydrofuro[3,4-c][1,7]naphthyridine-8-carboxamide |
| A392 | 4-amino-7-fluoro-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methyl-1,3-dihydrofuro[3,4-c] |
| | quinoline-8-carboxamide |
| A393 | 4-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methyl-1,3-dihydrofuro[3,4-c] |
| | quinoline-8-carboxamide |
| A394 | 2-amino-3-cyano-N-((S)-6-(6-((4-(2-(2-(((2S,4R)-1-((S)-3-ethyl-2-(1-fluorocyclopropane-1-carboxamido)pentanoyl)- |
| | 4-hydroxypyrrolidine-2-carboxamido)methyl)-5-(4-methylthiazol-5-yl)phenoxy)ethoxy)cyclohexyl)carbamoyl) |
| | pyridin-3-yl)-2,3-dihydrobenzofuran-3-yl)-N-methylquinoline-6-carboxamide |
| A395 | 2-amino-3-cyano-N-((S)-6-(6-((4-(2-(2-(((2S,4R)-1-((S)-2-(1-fluorocyclopropane-1-carboxamido)-2- |
| | (1-methylcyclopentyl)acetyl)-4-hydroxypyrrolidine-2-carboxamido)methyl)-5-(4-methylthiazol-5-yl) |
| | phenoxy)ethoxy)cyclohexyl)carbamoyl)yl)pyridin-3-yl)-2,3-dihydrobenzofuran-3-yl)-N-methylquinoline-6-carboxamide |
| A396 | 4-amino-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methyl-1H-pyrazolo[4,3-c]quinoline-8-carboxamide |
| A397 | 4-amino-N-((S)-6-(6-(((1S,4S)-4-(2-(2-(((2S,4R)-1-((S)-2-cyclopentyl-2-(1-fluorocyclopropane-1- |
| | carboxamido)acetyl)-4-hydoxypyrrolidine-2-carboxamido)methyl)-5-(4-methylthiazol-5-yl)phenoxy) |
| | ethoxy)cyclohexyl)carbamoyl)yl)pyridin-3-yl)-2,3-dihydrobenzofuran-3-yl)-N-methyl-1,3-dihydrofuro |
| | [3,4-c][1,7]naphthyridine-8-carboxamide |
| A398 | 2-amino-3-ethynyl-N-methyl-N-((3S)-6-(6-(4-(2,2,2-trifluoro-1-(1-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenyl)piperidin-4-yl)ethyl)piperazine-1-carbonyl)pyridin-3-yl)-2,3- |
| | dihydrobenzofuran-3-yl)quinoline-6-carboxamide |
| A399 | N1-(7-amino-1-methyl-1H-pyrazolo[3,4-c]pyridin-4-yl)-N2-((S)-6-(6-(4-((1-(2-(((2S,4R)-1-((S)-2- |
| | (1-fluorocyclopropane-1-carboxamido)-3,3-dimethylbutanoyl)-4-hydroxypyrrolidine-2-carboxamido) |
| | methyl)-5-(4-methylthiazol-5-yl)phenyl)piperidin-4-yl)methyl)piperazine-1-carbonyl)pyridin-3-yl)- |
| | 2,3-dihydrobenzofuran-3-yl)-N2-methyloxalamide |
| A400 | 3-cyclopropyl-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)yl)pyridin-3-yl)-2,3-dihydrobenzofuran-3-yl)-N-methyl-2-(methylamino)quinoline- |
| | 6-carboxamide |
| A401 | 2-amino-3-cyano-N-((S)-6-(6-((2-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethyl)- |
| | 2-azaspiro[3.5]nonan-7-yl)carbamoyl)pyridin-3-yl)-2,3-dihydrobenzofuran-3-yl)-N-methylquinoline-6-carboxamide |
| A402 | 2-amino-3-(difluoromethoxy)-N-((S)-6-(6-(((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)carbamoyl)pyridin-3-yl)-2,3-dihydrobenzofuran-3-yl)-N-methylquinoline-6-carboxamide |
| A403 | 2-amino-3-ethynyl-N-((S)-6-(2-((1S,4S)-4-(2-(2-(((2S,4R)-1-((S)-2-(1-fluorocyclopropane-1-carboxamido)-3,3- |
| | dimethylbutanoyl)-4-hydroxypyrrolidine-2-carboxamido)methyl)-5-(4-methylthiazol-5-yl)phenoxy)ethoxy) |
| | cyclohexyl)-1,3-dioxoisoindolin-5-yl)-2,3-dihydrobenzofuran-3-yl)-N-methylquinoline-6-carboxamide |

In some embodiments, the compound is not selected from Examples A292 and A329.

In some embodiments, the compound is not selected from Examples A292 and A329, or a pharmaceutically acceptable salt thereof.

In some embodiments, the compound is not selected from Examples A292 and A329, or a pharmaceutically acceptable salt, solvate, or stereoisomer thereof.

The compounds of the present disclosure may possess advantageous characteristics, as compared to known compounds, such as known PRMT5 degraders. For example, the compounds of the present disclosure may display more PRMT5 activity, more favorable pharmacokinetic properties (e.g., as measured by Cₘₐₓ, Tₘₐₓ, and/or AUC), and/or less interaction with other cellular targets (e.g., hepatic cellular transporter such as OATP1B1) and accordingly improved safety (e.g., drug-drug interaction). These beneficial properties of the compounds of the present disclosure may be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

### Pharmaceutically Acceptable Salts

In some embodiments, the compounds disclosed herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefor react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the disclosure may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the disclosure.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present disclosure.

In some embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers (Stereoisomers, Geometric Isomer, Tautomer, Etc.)

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is termed a “racemic mixture” or “racemate”.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In some embodiments, the weights are based upon total weight of all enantiomers or stereoisomers of the compound.

As used herein and unless otherwise indicated, the term “enantiomerically pure (R)-compound” refers to at least about 95% by weight (R)-compound and at most about 5% by weight(S) -compound, at least about 99% by weight (R)-compound and at most about 1% by weight(S)-compound, or at least about 99.9% by weight (R)-compound and at most about 0.1% by weight(S) -compound. In some embodiments, the weights are based upon total weight of compound.

As used herein and unless otherwise indicated, the term “enantiomerically pure(S)-compound” refers to at least about 95% by weight(S)-compound and at most about 5% by weight (R)-compound, at least about 99% by weight(S)-compound and at most about 1% by weight (R)-compound or at least about 99.9% by weight(S)-compound and at most about 0.1% by weight (R)-compound. In some embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure (R)-compound can comprise, for example, about 90% excipient and about 10% enantiomerically pure (R)-compound. In some embodiments, the enantiomerically pure (R)-compound in such compositions can, for example, comprise, at least about 95% by weight (R)-compound and at most about 5% by weight(S)-compound, by total weight of the compound. For example, a pharmaceutical composition comprising enantiomerically pure(S)-compound can comprise, for example, about 90% excipient and about 10% enantiomerically pure(S)-compound. In some embodiments, the enantiomerically pure(S)-compound in such compositions can, for example, comprise, at least about 95% by weight(S)-compound and at most about 5% by weight (R)-compound, by total weight of the compound. In some embodiments, the active ingredient can be formulated with little or no excipient or carrier.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

In some embodiments, the compounds described herein exist as geometric isomers. In some embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the disclosure.

In some embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the disclosure.

In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

In some embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest. All tautomeric forms of the compounds disclosed herein are contemplated and are within the scope of the disclosure. The exact ratio of the tautomers depends on several factors, including temperature, solvent, and pH.

### Conjugates

In some aspects, the present disclosure provides conjugates comprising the compound described herein, wherein the compound is directly or indirectly attached to a conjugate partner.

In some aspects, the present disclosure provides a compound described herein, wherein the compound is conjugated to a conjugate partner.

In some embodiments, the conjugate comprises a compound described herein directly attached (e.g., via a bond) to a conjugate partner.

In some embodiments, the conjugate comprises a compound described herein indirectly attached (e.g., via a linker moiety) to a conjugate partner.

In some embodiments, the conjugate comprises one or more compound described herein.

In some embodiments, the conjugate partner is an antibody or fragment thereof.

In some embodiments, the conjugate partner is an antibody.

In some embodiments, the conjugate partner is an antibody fragment.

In some embodiments, the conjugate partner is a half-life extending moiety (i.e., a moiety that increases the circulatory half-life of the conjugate in vivo, compared to the unconjugated compound).

In some embodiments, the compound described herein is conjugated to a conjugate partner.

In some embodiments, the compound described herein is conjugated to an antibody or fragment thereof.

In some embodiments, the compound described herein is conjugated to a half-life extending moiety.

In some embodiments, the present disclosure provides a compound for conjugating with a conjugate partner.

In some embodiments, the present disclosure provides a compound for conjugating with an antibody or fragment thereof.

In some embodiments, the present disclosure provides a compound for conjugating with a half-life extending moiety.

In some embodiments, the antibody and/or linker moiety of the conjugate is described in PCT Application No. PCT/US2023/085698; Hong, B. K. et al. J. Med. Chem. 2023, 66 (1), 140-148; Dragovich, P. S. Chem. Soc. Rev. 2022, 51 (10), 3886-3897; or Poudel, Y. B. et al. J. Med. Chem. 2024, 67 (18), 15996-16001 (each incorporated herein by reference).

In some embodiments, the half-life extending moiety comprises one or more moiety each independently selected from albumin, albumin binding domain, XTEN polypeptide, and polyalkylene glycol (PAO).

In some embodiments, the half-life extending moiety is selected from albumin, albumin binding domain, XTEN polypeptide, and polyalkylene glycol (PAO).

In some embodiments, the half-life extending moiety is albumin

In some embodiments, the half-life extending moiety is albumin binding domain.

In some embodiments, the half-life extending moiety is XTEN polypeptide.

In some embodiments, the half-life extending moiety is polyalkylene glycol (PAO).

In some embodiments, the PAO comprises a polymer of alkylene oxide.

In some embodiments, the PAO comprises a polymer of ethylene oxide.

In some embodiments, the PAO comprises a polymer of propylene oxide.

In some embodiments, the PAO is polypropylene glycol, polyethylene glycol (PEG), polyethylene glycol methyl ether (mPEG), or polyoxyethylenated polyol, or a copolymer (e.g., block copolymer) thereof.

In some embodiments, the PAO is polyethylene glycol (PEG) or polyethylene glycol methyl ether (mPEG).

In some embodiments, the PAO is polyethylene glycol (PEG).

In some embodiments, the PAO is polyethylene glycol methyl ether (mPEG).

In some embodiments, the PEG is polydisperse PEG, monodisperse PEG, or discrete PEG. In some embodiments, polydisperse PEGs are heterogeneous mixtures of sizes and molecular weights, whereas monodisperse PEGs are purified from heterogeneous mixtures and provide a single chain length and molecular weight. In some embodiments, the PEG units are discrete PEGs. In some embodiments, the discrete PEGs provide a single molecule with defined and specified chain length.

In some embodiments, the half-life extending moiety is described in Binder, U. et al. Expert Opinion on Biological Therapy, 2024, 25, 93-118, or Zaman, R. et al. Journal of Controlled Release, 2019, 301 (10), 176-189 (each incorporated herein by reference).

### Pharmaceutical Compositions

In some embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, the present disclosure provides pharmaceutical compositions comprising a compound described herein, or a pharmaceutically acceptable salt, solvate, or stereoisomer thereof, and a pharmaceutically acceptable excipient.

In some embodiments, the compound provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In some embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In some embodiments, the pharmaceutical composition is formulated for intravenous injection, oral administration, inhalation, nasal administration, topical administration, or ophthalmic administration. In some embodiments, the pharmaceutical composition is formulated for oral administration. In some embodiments, the pharmaceutical composition is formulated for intravenous injection. In some embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In some embodiments, the pharmaceutical composition is formulated as a tablet.

### Preparation and Characterization of the Compounds

The compounds of the present disclosure can be prepared in a number of ways well known to those skilled in the art of organic synthesis. By way of example, the compounds of the present disclosure can be synthesized using the methods described below, together with synthetic methods known in the art of synthetic organic chemistry, or variations thereon as appreciated by those skilled in the art. The compounds of the present disclosure (i.e., a compound of the present application (e.g., a compound of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Method

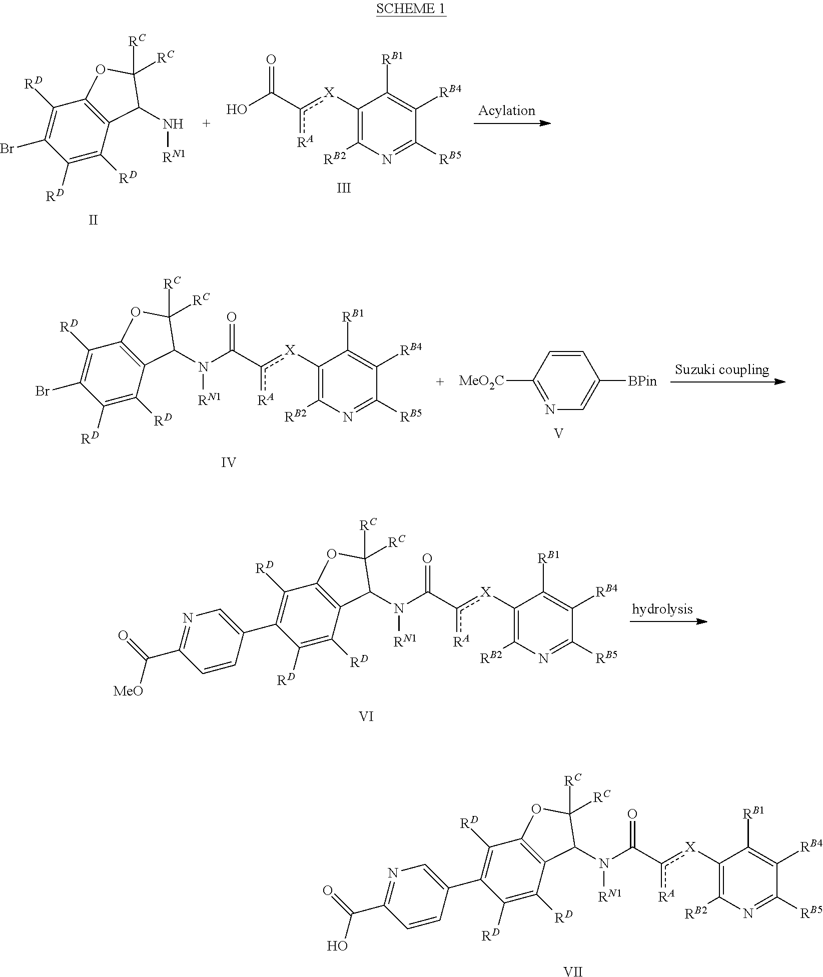

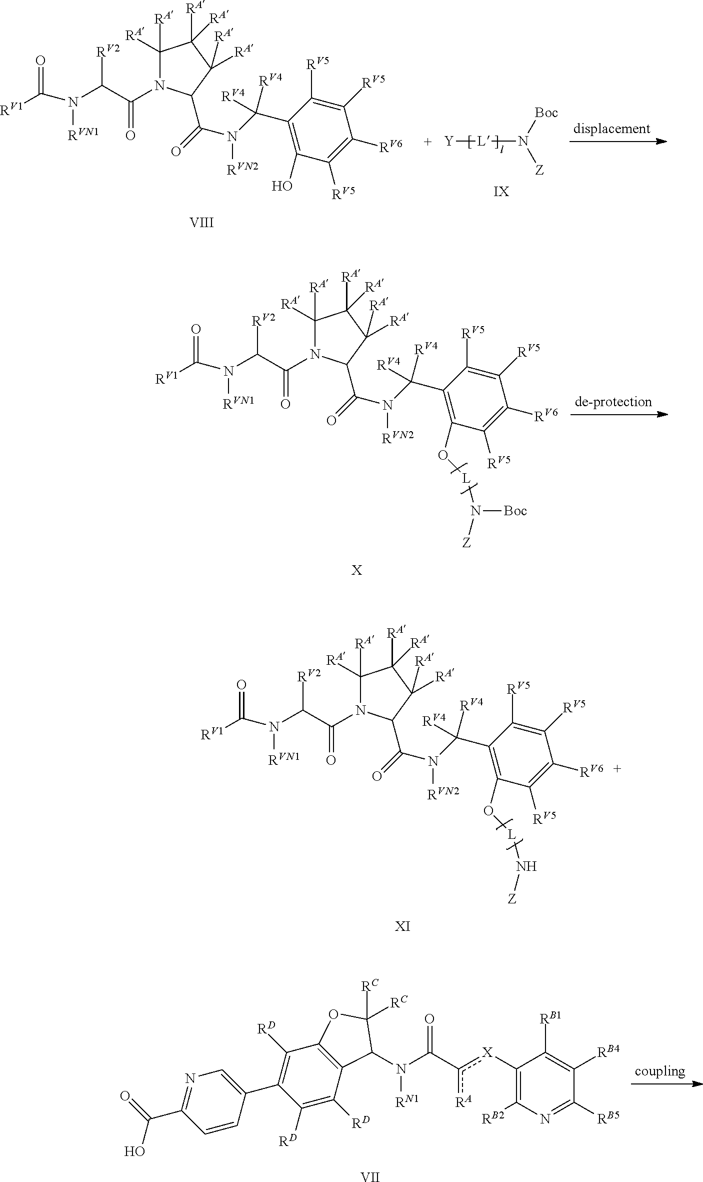

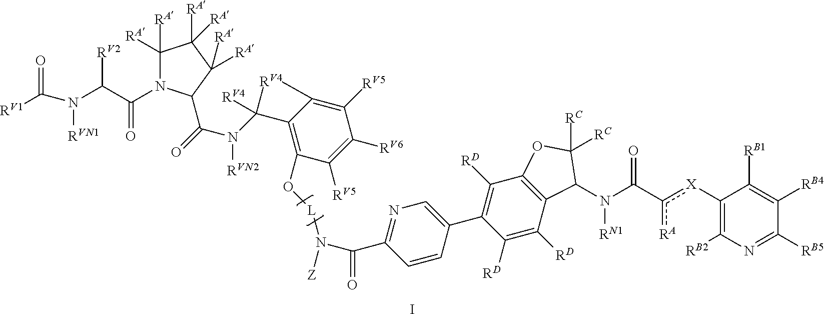

A compound of Formula I may be prepared according to the procedures shown in SCHEME 1.

According to SCHEME 1, a commercially available or prepared by known methods according to WO2024067433 of formula II is coupled with commercially available or prepared by known methods according to WO2022026892 of formula III; in a suitable solvent such as DME, DCM and the like; with a suitable coupling agent such as HATU, T3P and the like; at temperatures ranging from 0° C. to 60° C.; to provide the coupled compound of formula IV. A compound of formula IV is coupled with a commercially available pinacol borate ester of formula V; with a suitable catalyst such as PdCl₂(dppf), Pd(Ph₃P)₄ and the like; with a suitable base such as potassium acetate, sodium carbonate, and the like; in a suitable solvent such as 1,4-dioxane, DMSO and the like; at temperatures ranging from 60° C. to 120° C.; to provide coupled compound of formula VI. A coupled compound of formula VI is hydrolyzed with a suitable base such as LiOH, NaOH and the like; in a suitable solvent such as MeOH, THE, water and the like; at temperatures ranging from 25° C. to 80° C.; to provide hydrolyzed compound of formula VII.

A commercially available or prepared according to WO20230181555 compound of formula VIII is treated with a commercially available or prepared according to WO20230181555 or WO2018189664 compound of formula IX, under a suitable base such as Cs₂CO₃, K₂CO₃ and the like; in a suitable solvent such as DMF, DMSO and the like; at temperatures ranging from 60° C. to 120° C.; to provide coupled compound of formula X. A compound of formula X is treated with an acidic solvent such as TFA, 6 N HCl and the like in a suitable solvent such as 1,4-dioxane, DCM and the like; at temperatures ranging from 0° C. to 40° C.; to provide a primary amine compound of formula XI.

An amine compound of formula XI is reacted with an acidic compound of formula VII; in a suitable solvent such as DMF, DCM and the like; with a suitable coupling agent such as HATU, T3P and the like; at temperatures ranging from 0° C. to 60° C.; to provide the coupled compound of formula I.

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J.C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In some embodiments, each of the intermediates prepared in the Schemes herein are considered embodiments of the present disclosure.

In some embodiments, each of the intermediates prepared in the Examples herein are considered embodiments of the present disclosure.

In some embodiments, each synthetic step as disclosed in the Schemes herein is separately considered as part of the present disclosure.

In some embodiments, each synthetic step as disclosed in the Examples herein is separately considered as part of the present disclosure.

### Biological Assays

The biological activities of the compounds of the present application can be assessed with methods and assays known in the art.

In some embodiments, protein degradation is measured using HiBIT Assay. For example, PRMT5 degradation potency and Dₘₐₓ were determined using in HiBit technology in HiBit MTAP KO and MTAP WT cells. HCT116 cells +/−MTAP (Creative Biogene (CSC-RT2701) were genetically modified via CRISPR/Cas9 to fuse HiBiT to the carboxy terminus of PRMT5. Cells were cultured in McCoy's 5A containing 10% FBS and 1% (Vol: Vol) penicillin-streptomycin. At the time of experiment, cells were seeded at a density of 1250/25 μL/well in a 384-well plate (Corning; cat #3571) 24 hrs prior to compound treatment. Investigational compounds were prepared at 3 mM top concentration in 100% DMSO and an 11-point 3-fold serial dilution done in DMSO. 25 nL of serial diluted compounds were transferred to wells containing cell in 25 μL of media for a final top concentration of 3 μM. An equal volume of DMSO was added to control wells. Each treatment was performed in duplicate. Cells were incubated at 37° C.+5% CO₂ in a cell culture incubator for 18 hours. At the end of experiment, levels of PRMT5 were evaluated with Nano-Glo® HiBIT Lytic Detection Assay (Promega N3050). Assay reagent was prepared according to the manual provided with the Promega kit. 25 μl of this assay solution was added to each well. Luminescence from each well was measured with Envision plate reader (Perkin Elmer). Dose response curves were fit using Perkin Elmer Signals Lead Discovery software. In some embodiments, the biological assay is described in the Examples herein.

### Methods of Use

In some aspects, the present disclosure provides methods of degrading a PRMT5 protein in a subject, comprising administering to the subject a compound disclosed herein.

In some aspects, the present disclosure provides uses of a compound disclosed herein in the manufacture of a medicament for degrading a PRMT5 protein in a subject.

In some aspects, the present disclosure provides compounds disclosed herein for use in degrading a PRMT5 protein in a subject.

In some aspects, the present disclosure provides methods of reducing the amount of a SMARCA2 or SMARCA4 protein in a subject (e.g., in a biological sample (e.g., a cell or a tissue) obtained from the subject), comprising administering to the subject a compound disclosed herein.

In some aspects, the present disclosure provides uses of a compound disclosed herein in the manufacture of a medicament for reducing the amount of a PRMT5 protein in a subject (e.g., in a biological sample (e.g., a cell or a tissue) obtained from the subject).

In some aspects, the present disclosure provides compounds disclosed herein for use in reducing the amount of a PRMT5 protein in a subject (e.g., in a biological sample (e.g., a cell or a tissue) obtained from the subject).

In some aspects, the present disclosure provides methods of treating or preventing a disease or disorder in a subject in need thereof, comprising administering to the subject a compound disclosed herein (e.g., in a therapeutically effective amount).

In some aspects, the present disclosure provides methods of treating a disease or disorder in a subject in need thereof, comprising administering to the subject a compound disclosed herein (e.g., in a therapeutically effective amount).

In some aspects, the present disclosure provides uses of a compound disclosed herein in the manufacture of a medicament for treating or preventing a disease or disorder in a subject in need thereof.

In some aspects, the present disclosure provides uses of a compound disclosed herein in the manufacture of a medicament for treating a disease or disorder in a subject in need thereof.

In some aspects, the present disclosure provides compounds disclosed herein for use in treating or preventing a disease or disorder in a subject in need thereof.

In some aspects, the present disclosure provides compounds disclosed herein for use in treating a disease or disorder in a subject in need thereof.

In some embodiments, the disease or disorder is a PRMT5 protein-mediated disease or disorder, such as breast, lung, colorectal, and pancreatic cancers, as well as glioblastoma, neuroblastoma, and hematologic malignancies.

In some embodiments, the disease or disorder is cancer.

In some embodiments, the cancer is non-small cell lung cancer, small-cell lung cancer, colorectal cancer, bladder cancer, glioma, breast cancer, melanoma, non-melanoma skin cancer, endometrial cancer, esophagogastric cancer, pancreatic cancer, hepatobiliary cancer, soft tissue sarcoma, ovarian cancer, head and neck cancer, renal cell carcinoma, bone cancer, non-Hodgkin lymphoma, prostate cancer, embryonal tumor, germ cell tumor, cervical cancer, thyroid cancer, salivary gland cancer, gastrointestinal neuroendocrine tumor, uterine sarcoma, gastrointestinal stromal tumor, CNS cancer, thymic tumor, Adrenocortical carcinoma, appendiceal cancer, small bowel cancer, or penile cancer.

In some embodiments, the cancer is selected from NSCLC adenocarcinoma (LUAD), NSCL squamous cell carcinoma (LUSC), liver hepatocellular carcinoma (LIHC), uterine corpus endometrial carcinoma (UCEC), esophageal carcinoma (ESCA), skin cutaneous melanoma (SKCM), stomach adenocarcinoma (STAD), colon adenocarcinoma (COAD), bladder urothelial carcinoma (BLCA), and uterine carcinosarcoma (UCS).

In some embodiments, the cancer is selected from NSCLC adenocarcinoma (LUAD), NSCL squamous cell carcinoma (LUSC), liver hepatocellular carcinoma (LIHC), and uterine corpus endometrial carcinoma (UCEC).

In some embodiments, the cancer includes, but is not limited to, one or more of the cancers of Table A.

TABLE A

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal carcinoma | endocrine gland | endodermal sinus | enteropathy- |
| | neoplasm | tumor | associated T-cell |
| | | | lymphoma |
| esophageal cancer | fetus in fetu | fibroma | fibrosarcoma |
| follicular lymphoma | follicular thyroid | ganglioneuroma | gastrointestinal |
| | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck cancer | hemangiopericytoma | hematological cancer | hepatoblastoma |
| hepatosplenic T-cell | Hodgkin's lymphoma | non-Hodgkin's | invasive lobular |
| lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton | mantle cell |
| histiocytoma | nerve sheath tumor | tumor | lymphoma |
| marginal zone B-cell | mast cell leukemia | mediastinal germ cell | medullary carcinoma |
| lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid liposarcoma | myxoma | myxosarcoma | nasopharyngeal |
| | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | primary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal zone | squamous cell | synovial sarcoma | Sezary's disease |
| lymphoma | carcinoma | | |
| small intestine cancer | squamous carcinoma | stomach cancer | T-cell lymphoma |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In some embodiments, the cancer is a solid tumor. In some embodiments, the cancer is a hematological cancer. Exemplary hematological cancers include, but are not limited to, the cancers listed in Table B. In some embodiments, the hematological cancer is acute lymphocytic leukemia, chronic lymphocytic leukemia (including B-cell chronic lymphocytic leukemia), or acute myeloid leukemia.

TABLE B

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic |
| | lymphoma |
| Waldenstrom's macroglobulinemia | T-cell lymphoma |
| (WM) | |
| diffuse large B-cell lymphoma | mast cell leukemia |
| (DLBCL) | |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell |
| | lymphoma |
| Richter's transformation | |

In some embodiments, the subject is a mammal.

In some embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.F. Eliel, Ed., Univ. of Notre Dame Press, Notre Dame, IN 1972).

The disclosure additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₃₋₆ alkyl.

The following terms are intended to have the meanings presented therewith below and are useful in understanding the description and intended scope of the present disclosure. When describing the disclosure, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, which is also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl(C₁), ethyl(C₂), n-propyl(C₃), isopropyl(C₃), n-butyl(C₄), tert-butyl(C₄), sec-butyl(C₄), isobutyl(C₄), n-pentyl(C₅), 3-pentanyl(C₅), amyl(C₅), neopentyl(C₅), 3-methyl-2-butanyl(C₅), tertiary amyl(C₅), and n-hexyl(C₆). Additional examples of alkyl groups include n-heptyl(C₂), n-octyl(C₄) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In some embodiments, the alkyl group is unsubstituted C₁₋₁₀ alkyl (e.g., —CH₃). In some embodiments, the alkyl group is substituted C₁₋₁₀ alkyl. Common alkyl abbreviations include Me(—CH₃), Et(—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkylene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH (CH₃)—, —C(CH₃)₂CH₂—, —CH₂C(CH₃)₂—), substituted propylene (—CH(CH₃)CH₂CH₂—, —CH₂CH (CH₃)CH₂—, —CH₂CH₂CH (CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds)(“C₂₋₂₀ alkenyl”). In some embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl(C₂), 1-propenyl(C₃), 2-propenyl(C₃), 1-butenyl(C₄), 2-butenyl(C₄), butadienyl(C₄), and the like. Examples of C₂₋₆ alkenyl groups include the aforementioned C₂₋₄ alkenyl groups as well as pentenyl(C₅), pentadienyl(C₅), hexenyl(C₆), and the like. Additional examples of alkenyl include heptenyl(C₇), octenyl(C₈), octatrienyl(C₅), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In some embodiments, the alkenyl group is unsubstituted C₂₋₁₀ alkenyl. In some embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH—CHCH₂—, —CH₂—CH═CH—). Exemplary substituted divalent alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)—CHCH₂—, —CH═C(CH₃)CH₂—, —CH═CHCH (CH₃)—, —CH═CHC(CH₃)₂-, —CH (CH₃)—CH—CH—, —C(CH₃)₂-CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds)(“C₂₋₂₀ alkynyl”). In some embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl(C₂), 1-propynyl(C₃), 2-propynyl(C₃), 1-butynyl(C₄), 2-butynyl(C₄), and the like. Examples of C₂₋₆ alkenyl groups include the aforementioned C₂₋₄ alkynyl groups as well as pentynyl(C₅), hexynyl(C₆), and the like. Additional examples of alkynyl include heptynyl(C₂), octynyl(C₅), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In some embodiments, the alkynyl group is unsubstituted C₂₋₁₀ alkynyl. In some embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In some embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and/or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In some embodiments, the heteroalkyl group is an unsubstituted heteroC₁₋₁₀ alkyl. In some embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment, In some embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and lor 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In some embodiments, the heteroalkenyl group is an unsubstituted heteroC₂₋₁₀ alkenyl. In some embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In some embodiments, a heteroalkynyl group refers to a group having from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆ alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and lor 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In some embodiments, the heteroalkynyl group is an unsubstituted heteroC₂₋₁₀ alkynyl. In some embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In some embodiments, the aryl group is unsubstituted C₆₋₁₄ aryl. In some embodiments, the aryl group is substituted C₆₋₁₄ aryl.

“Arylene” as used herein, refers to an aryl group wherein two hydrogens are removed to provide a divalent radical. When a range or number of carbons is provided for a particular “arylene” group, it is understood that the range or number refers to the range or number of carbons in the aryl group. An “arylene” group may be substituted or unsubstituted with one or more substituents as described herein.

“Heteroaryl” refers to a radical of a 5- to 14-membered monocyclic or polycyclic 4n+2 aromatic ring system (e.g., having 6, 10, or 14 π electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In some embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In some embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In some embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In some embodiments, the 5- to 6-membered heteroaryl has 1-3 ring heteroatoms independently selected from nitrogen, oxygen, and sulfur. In some embodiments, the 5- to 6-membered heteroaryl has 1-2 ring heteroatoms independently selected from nitrogen, oxygen, and sulfur. In some embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In some embodiments, the heteroaryl group is unsubstituted 5- to 14-membered heteroaryl. In some embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroarylene” as used herein, refers to a heteroaryl group wherein two hydrogens are removed to provide a divalent radical. When a range or number of ring members is provided for a particular “heteroarylene” group, it is understood that the range or number refers to the number of ring members in the heteroaryl group. A “heteroarylene” group may be substituted or unsubstituted with one or more substituents as described herein.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In some embodiments, a carbocyclyl group has 3 to 10 ring carbon atoms (“C₃-10 carbocyclyl”). In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 12 ring carbon atoms (“C₅₋₁₂ carbocyclyl”). In some embodiments, a carbocyclyl group has 5 to 10 ring carbon atoms (“C₅₋₁₀ carbocyclyl”). In some embodiments, a carbocyclyl group has 5 to 8 ring carbon atoms (“C₅₋₈ carbocyclyl”). In some embodiments, a carbocyclyl group has 5 or 6 ring carbon atoms (“C₅₋₆ carbocyclyl”). Exemplary C₃₋₆ carbocyclyl include, without limitation, cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), and the like. Exemplary C₃₋₈ carbocyclyl include, without limitation, the aforementioned C₃₋₆ carbocyclyl groups as well as cycloheptyl(C₂), cycloheptenyl(C₂), cycloheptadienyl(C₂), cycloheptatrienyl(C₂), cyclooctyl(C₂), cyclooctenyl(C₄), bicyclo[2.2.1]heptanyl(C₇), bicyclo[2.2.2]octanyl(C₅), and the like. Exemplary C₃₋₁₀ carbocyclyl include, without limitation, the aforementioned C₃₋₈ carbocyclyl groups as well as cyclononyl(C₉), cyclononenyl(C₉), cyclodecyl(C₁₀), cyclodecenyl(C₁₀), octahydro-1H-indenyl(C₉), decahydronaphthalenyl(C₁₀), spiro[4.5]decanyl(C₁₀), and the like.

In some embodiments, “carbocyclyl” is a monocyclic, saturated carbocyclyl group having from 3 to 12 ring carbon atoms (“C₃₋₁₂ carbocyclyl”). In some embodiments, “carbocyclyl” is a monocyclic, saturated carbocyclyl group having from 3 to 10 ring carbon atoms (“C₃-10 carbocyclyl”). In some embodiments, “carbocyclyl” is a monocyclic, saturated carbocyclyl group having from 3 to 8 ring carbon atoms (“C₃-8 carbocyclyl”). In some embodiments, “carbocyclyl” is a monocyclic, saturated carbocyclyl group having from 3 to 6 ring carbon atoms (“C₃₋₆ carbocyclyl”). In some embodiments, “carbocyclyl” is a monocyclic, saturated carbocyclyl group having from 5 to 12 ring carbon atoms (“C₃₋₁₂ carbocyclyl”). In some embodiments, a carbocyclyl group has 5 to 10 ring carbon atoms (“C₅₋₁₀ carbocyclyl”). In some embodiments, a carbocyclyl group has 5 to 8 ring carbon atoms (“C₅₋₈ carbocyclyl”). In some embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl(C₅) and cyclohexyl(C₅). Examples of C₃₋₆ carbocyclyl include the aforementioned C₅₋₆ carbocyclyl groups as well as cyclopropyl(C₃) and cyclobutyl(C₄). Examples of C₃₋₈ carbocyclyl include the aforementioned C₃₋₆ carbocyclyl groups as well as cycloheptyl(C₂) and cyclooctyl(C₈). Unless otherwise specified, each instance of a carbocyclyl group is independently unsubstituted (an “unsubstituted carbocyclyl”) or substituted (a “substituted carbocyclyl”) with one or more substituents. In some embodiments, the carbocyclyl group is unsubstituted C₃₋₁₂ carbocyclyl. In some embodiments, the carbocyclyl group is substituted C₃₋₁₂ carbocyclyl.

As the foregoing examples illustrate, In some embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In some embodiments, the carbocyclyl group is unsubstituted C₃₋₁₂ carbocyclyl. In some embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocycle group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”).

In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a Ce aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In some embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In some embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In some embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In some embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In some embodiments, the 5- to 6-membered heterocyclyl has 1-3 ring heteroatoms selected from nitrogen, oxygen, and sulfur. In some embodiments, the 5- to 6-membered heterocyclyl has 1-2 ring heteroatoms selected from nitrogen, oxygen, and sulfur. In some embodiments, the 5- to 6-membered heterocyclyl has one ring heteroatom selected from nitrogen, oxygen, and sulfur.

As the foregoing examples illustrate, In some embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In some embodiments, the heterocyclyl group is unsubstituted 3- to 12-membered heterocyclyl. In some embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocycle group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group-OR, wherein R is alkyl as defined herein. C₁₋₆ lkoxy refers to the group-OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro (Cl), bromo (Br), and iodo (I). In some embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl (Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl (Moz or MeOZ), 2,2,2-trichloroehtoxycarbonyl (Troc), and benzyl carbamate (Cbz)), esters (e.g., acetyl (Ac); benzoyl (Bz), trifluoroacetyl, and phthalimide), amines (e.g., benzyl (Bn), p-methoxybenzyl (PMB), p-methoxyphenyl (PMP), and triphenylmethyl (trityl)), and sulfonamides (e.g., tosyl (Ts), N-alkyl nitrobenzenesulfonamides (Nosyl), and 2-nitrophenylsulfenyl (Nps)).

Common types of thiol-protecting groups include but not limited to sulfide (e.g., p-methylbenzyl (Meb), t-butyl, acetamidomethyl (Acm), and triphenylmethyl (Trityl)).

Common types of carboxylic acid-protecting groups include but not limited to esters (e.g., methyl ester, triphenylmethyl (Trityl), t-butyl ester, benzyl ester (Bn), S-t-butyl ester, silyl esters, and orthoesters) and oxazoline.

These and other exemplary substituents are described in more detail in the Detailed Description, Examples, and claims. The disclosure is not intended to be limited in any manner by the above exemplary listing of substituents.

### Other Definitions

“Antibody” is used in the broadest sense and specifically encompasses monoclonal antibodies (including full length monoclonal antibodies), polyclonal antibodies, multispecific antibodies (e.g., bispecific antibodies), and antibody fragments so long as they exhibit the desired biological activity. “Antibody fragment” and all grammatical variants thereof as used herein are defined as a portion of an intact antibody comprising the antigen binding site or variable region of the intact antibody, wherein the portion is free of the constant heavy chain domains (i.e., CH₂, CH₃, and CH₄, depending on antibody isotype) of the Fc region of the intact antibody. Examples of antibody fragments include Fab, Fab, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). “Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (VL and VH, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells. An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells. “Antibody construct” refers to an antibody or a fusion protein comprising (i) an antigen binding domain and (ii) an Fc domain. In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the VL, VH, CL, and CH₁ domains, (ii) a F(ab′)₂ fragment, which is a bivalent fragment comprising two Fab fragments linked by a disulfide bridge at the hinge region, (iii) a Fv fragment consisting of the VL and VH domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., VL and VH) joined by a synthetic linker which enables the two domains to be synthesized as a single polypeptide chain. The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a T-cell receptor, so as to provide a T-cell receptor like construct that binds TROP2. In yet another embodiment, the antibody fragment is part of a bispecific T-cell engager (BiTEs) comprising a CD1 or CD3 binding domain and linker. “Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics. The terms “Fc receptor” or “FcR” refer to a receptor that binds to the Fc region of an antibody. There are three main classes of Fc receptors: (1) FcγR which bind to IgG, (2) FcαR which binds to IgA, and (3) FcaR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal,

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In some embodiments, the number or numerical range vary by 1%, 2%, 3%, 4%, 5%, 6%, 7%, 8%, 9%, 10%, 11%, 12%, 13%, 14%, or 15% of the stated number or numerical range. In some embodiments, the number or numerical range vary by 1%, 2%, 3%, 4%, or 5% of the stated number or numerical range. In some embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, when a moiety is “optionally substituted with one or more A, B, C, D, or E,” it is understood that the moiety is optionally substituted with one more substituent, wherein each substituent is independently selected from A, B, C, D, and E.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### Exemplary Embodiments

Exemplary Embodiment No. 1. A Compound of Formula I

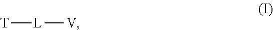

or a pharmaceutically acceptable salt, solvate, or stereoisomer thereof, wherein:
T is of Formula 1-1

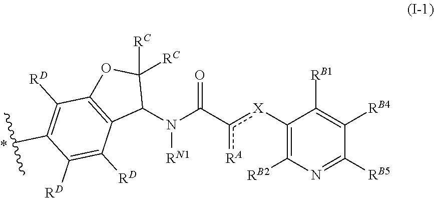

wherein:
* denotes attachment to L;
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
R^{B1}, R^{B2}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, or —NH₂; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl;
R^{N1} is hydrogen, C₁₋₆ alkyl, or C₃₋₁₂ carbocyclyl, wherein the C₁₋₆ alkyl or C₃₋₁₂ carbocyclyl is optionally substituted with one or more halogen, —CN, —NO₂, —OH, —NH₂, or C₃₋₁₂ carbocyclyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
each R^{D} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
L is of Formula I-2

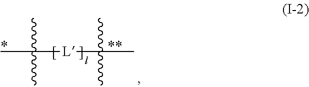

wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N(R^{L′})—, —O—, —O—(C₁₋₆ alkylene)-, —(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O—(C₁₋₆ alkylene)-, —(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more halogen, —CN, —NO₂, —OH, or —NH₂;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(═O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
1 is an integer selected from 0 to 10;
V is of Formula I-3

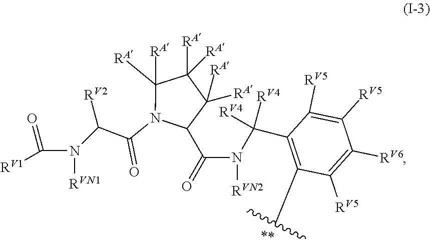

wherein:
* denotes attachment to L;
R^{V1} is hydrogen, C₁₋₆ alkyl, C₃₋₆ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, or —NH₂;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen or C₁₋₆ alkyl, wherein the C₁₋₆ alkyl is optionally substituted with one or more halogen, —CN, —NO₂, —OH, or —NH₂;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
R^{V6} is C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, C₁₋₆ alkyl, C₂₋₆ alkenyl, or C₂₋₆ alkynyl.

Exemplary Embodiment No. 2. The compound of Exemplary Embodiment No. 1, wherein X is N(R^{X1}), and R^{A} is oxo.

Exemplary Embodiment No. 3. The compound of Exemplary Embodiment No. 1, wherein X is C(R^{X2}), and R^{A} and R^{B2}, together with the intervening atoms to which they are attached, form C₆ aryl or 6-membered heteroaryl, wherein the C₆ aryl or 6-membered heteroaryl is optionally substituted with one or more halogen, —CN, —NO₂, —OH, or —NH₂.

Exemplary Embodiment No. 4. The compound of Exemplary Embodiment No. 3, wherein R^{A} and R^{B2}, together with the intervening atoms to which they are attached, form C₆ aryl or pyridinyl.

Exemplary Embodiment No. 5. The compound of any one of Exemplary Embodiment Nos. 1-4, wherein R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂-6 alkynyl.

Exemplary Embodiment No. 6. The compound of Exemplary Embodiment No. 5, wherein R^{B1} and R^{B4}, together with the intervening atoms to which they are attached, form dihydrofuran or pyrazole optionally substituted with one or more halogen or C₁₋₆ alkyl.

Exemplary Embodiment No. 7. The compound of any one of Exemplary Embodiment Nos. 1-4, wherein R^{B1} is hydrogen or C₁₋₆ alkyl, and R^{B4} is halogen, —CN, C₁₋₆ alkyl optionally substituted with one or more-OH, C₂₋₆ alkynyl, C₃₋₁₂ carbocyclyl, or 3- to 12-membered heterocyclyl.

Exemplary Embodiment No. 8. The compound of any one of Exemplary Embodiment Nos. 1-7, wherein R^{B5} is hydrogen or —NH₂.

Exemplary Embodiment No. 9. The compound of any one of Exemplary Embodiment Nos. 1-8, wherein each R^{C} is independently hydrogen.

Exemplary Embodiment No. 10. The compound of any one of Exemplary Embodiment Nos. 1-9, wherein R^{N1} is C₁₋₆ alkyl optionally substituted with one or more C₃₋₁₂ carbocyclyl.

Exemplary Embodiment No. 11. The compound of any one of Exemplary Embodiment Nos. 1-9, wherein R^{N1} is C₃₋₁₂ carbocyclyl.

Exemplary Embodiment No. 12. The compound of any one of Exemplary Embodiment Nos. 1-11, wherein each R^{D} is independently hydrogen.

Exemplary Embodiment No. 13. The compound of any one of Exemplary Embodiment Nos. 1-12, wherein T is of Formula I-1-a or I-1-b

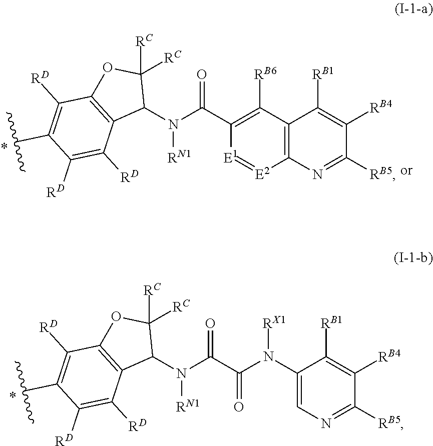

wherein:
E¹ is N or CR^{B7};
E² is N or CR^{B8}, and
R^{B6}, R^{B7}, and R^{B8} are independently hydrogen, halogen, —CN, —NO₂, —OH, or —NH₂.

Exemplary Embodiment No. 14. The compound of any one of Exemplary Embodiment Nos. 1-13, wherein each L′ is independently selected from —C(═O)—, —C(═O)N(R^{L′})—, —N(R^{L})C(═O)—, —N(R^{L′})—, —O—, —O—(C₁₋₆ alkylene)-, —(C₁₋₆ alkylene)-O—, C₁₋₆ alkylene, C₁₋₆ heteroalkylene, C₃₋₁₂ carbocyclylene, 3- to 12-membered heterocyclylene, and 5- to 10-membered heteroarylene.

Exemplary Embodiment No. 15. The compound of any one of Exemplary Embodiment Nos. 1-14, wherein L is **—[O—(C₁₋₆ alkylene)]₁₋₈—N(R^{L′})C(═O)-(5- to 10-membered heteroarylene)-*, **—O—[(C₁₋₆ alkylene)]₁₋₃—N(R^{L′})C(═O)-(5- to 6-membered heteroarylene)-*, **—[O—(C₁₋₆ alkylene)]₁₋₇-(3- to 12-membered heterocyclylene)-C(═O)-(5- to 10-membered heteroarylene)-*, **—[O—(C₁₋₆ alkylene)]₁₋₆—O—(C₃₋₁₂ carbocyclylene)-N(R1′)C(═O)-(5- to 10-membered heteroarylene)-*, or **—O—[(C₁₋₆ heteroalkylene)]_{1*3}—N(R^{L′})C(═O)-(5- to 10-membered heteroarylene)-*.

Exemplary Embodiment No. 16. The compound of Exemplary Embodiment No. 15, wherein L is **—[O—(C₁₋₆ alkylene)]₁₋₈—N(H)C(═O)-(pyridinylene)-*, **—O—[(C₁₋₆ alkylene)]₁₋₃-N (H)C(═O)-(pyridinylene)-*, **—[O—(C₁₋₆ alkylene)]₁₋₇-(3- to 12-membered heterocyclylene)-C(═O)-(pyridinylene)-*, **—[O—(C₁₋₆ alkylene)]₁₋₆—O—(C₃₋₁₂ carbocyclylene)-N(H)C(═O)-(pyridinylene) —*, or **—O—[(C₁₋₆ heteroalkylene)]₁₋₃—N(H)C(═O)-(pyridinylene)-*.

Exemplary Embodiment No. 17. The compound of any one of Exemplary Embodiment Nos. 1-16, wherein R^{V1} is C₁₋₆ alkyl or C₃₋₆ carbocyclyl, wherein the C₁₋₆ alkyl or C₃₋₆ carbocyclyl is optionally substituted with one or more halogen.

Exemplary Embodiment No. 18. The compound of any one of Exemplary Embodiment Nos. 1-17, wherein R^{VN1} and R^{VN2} are each independently hydrogen.

Exemplary Embodiment No. 19. The compound of any one of Exemplary Embodiment Nos. 1-18, wherein R^{V2} is C₁₋₆ alkyl.

Exemplary Embodiment No. 20. The compound of any one of Exemplary Embodiment Nos. 1-19, wherein one R^{A′} is —OH, and each of the remaining R^{A′} is independently hydrogen.

Exemplary Embodiment No. 21. The compound of any one of Exemplary Embodiment Nos. 1-20, wherein each R^{V4} is independently hydrogen.

Exemplary Embodiment No. 22. The compound of any one of Exemplary Embodiment Nos. 1-21, wherein each R^{V5} is independently hydrogen.

Exemplary Embodiment No. 23. The compound of any one of Exemplary Embodiment Nos. 1-22, wherein R^{V6} is C₂₋₆ alkynyl or 5- to 10-membered heteroaryl optionally substituted with one or more C₁₋₆ alkyl.

Exemplary Embodiment No. 24. The compound of any one of Exemplary Embodiment Nos. 1-23, wherein V is of Formula 1-3-a

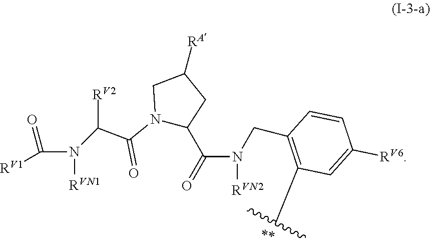

Exemplary Embodiment No. 25. A compound selected from the compounds in Table 1, or a pharmaceutically acceptable salt thereof.

Exemplary Embodiment No. 26. A pharmaceutical composition comprising the compound of any one of Exemplary Embodiment Nos. 1-25, and a pharmaceutically acceptable excipient.

Exemplary Embodiment No. 27. A method of treating a disease or disorder comprising administering to a patient in need thereof a compound of any one of Exemplary Embodiment Nos. 1-25.

Exemplary Embodiment No. 28. Use of a compound of any one of Exemplary Embodiment Nos. 1-25 in the manufacture of a medicament for treating a disease or disorder.

Exemplary Embodiment No. 29. A compound of any one of Exemplary Embodiment Nos. 1-25 for use in treating a disease or disorder.

Exemplary Embodiment No. 30. The method, use, or compound for use of any one of Exemplary Embodiment Nos. 27-29, wherein the disease or disorder is a PRMT5 protein-mediated disease or disorder.

Exemplary Embodiment No. 31. The method, use, or compound for use of any one of Exemplary Embodiment Nos. 27-30, wherein the disease or disorder is cancer.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

### I. Synthesis and Characterization of Compounds

In the following examples, the chemical reagents were purchased from commercial sources (such as Alfa, Acros, Sigma Aldrich, TCI and Shanghai Chemical Reagent Company), and used without further purification.

In obtaining the compounds described in the examples below and the corresponding analytical data, the following experimental and analytical protocols were followed unless otherwise indicated.

Unless otherwise stated, reaction mixtures were magnetically stirred at room temperature (rt) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Compound purification was carried out as needed using a variety of traditional methods including, but not limited to, preparative chromatography under acidic, neutral, or basic conditions using either normal phase or reverse phase HPLC or flash columns or Prep-TLC plates.

Flash chromatography was performed on a Biotage Isolera One via column with silica gel particles of 100-200 mesh or 200-300 mesh. Analytical and preparative thin-layer chromatography was performed using silica gel 60 GF254 plates. Normal-phase silica gel chromatography (FCC) was also performed on silica gel (SiO₂) using prepacked cartridges.

Preparative reverse-phase high performance liquid chromatography (RP HPLC) was performed on either:

### Method A

Prep-HPLC with YMC-Actus Triart 18C(5 μm, 20×250 mm), and mobile phase of 5-99% ACN in water (0.1% HCOOH) over 10 min and then hold at 100% ACN for 2 min, at a flow rate of 25 mL/min; or

### Method B

Preparative supercritical fluid high performance liquid chromatography (SFC) was performed either on a Thar 80 Prep-SFC system, or Waters 80Q Prep-SFC system from Waters. The ABPR was set to 100 bar to keep the CO₂ in SF conditions, and the flow rate may verify according to the compound characteristics, with a flow rate ranging from 50 g/min to 70 g/min. The column temperature was ambient temperature.

Nuclear magnetic resonance (NMR) spectra were recorded using Brucker AVANCE NEO 400 MHz at around 20-30° C. unless otherwise specified. The following abbreviations are used: s, singlet; d, doublet; t, triplet; q, quartet; m, multiplet; dd, doublet of doublets; ddd, doublet of doublet of doublets; dt, doublet of triplets; bs, broad signal. Chemical shifts were reported in parts per million (ppm, 8) downfield from tetramethylsilane. It will be understood that for compounds comprising an exchangeable proton, said proton may or may not be visible on an NMR spectrum depending on the choice of solvent used for running the NMR spectrum and the concentration of the compound in the solution.

Mass spectra (MS) were obtained on a SHIMADZU LCMS-2020 MSD using electrospray ionization (ESI) in positive mode unless otherwise indicated. Calculated (calcd.) mass corresponds to the exact mass.

Chemical names were generated using ChemDraw Ultra 12.0, ChemDraw Ultra 14.0, ChemDraw Ultra 20.0 (CambridgeSoft Corp., Cambridge, MA) or ACD/Name Version 10.01 (Advanced Chemistry).

Compounds designated as R or S are enantiopure compounds where the absolute configuration was determined.

### Intermediate 1. (S)-5-(3-(4-(bis(tert-butoxycarbonyl)amino)-N,1-dimethyl-1H-pyrazolo[4,3-c]quinoline-8-carboxamido)-2,3-dihydrobenzofuran-6-yl)picolinic acid

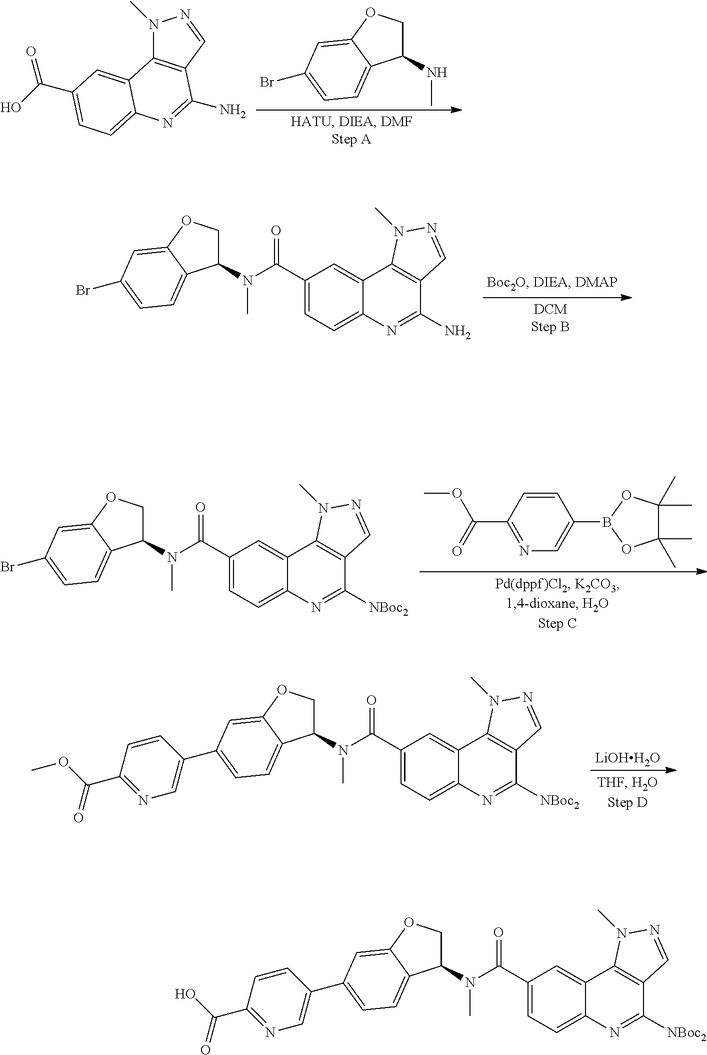

Step A: (S)-4-amino-N-(6-bromo-2,3-dihydrobenzofuran-3-yl)-N,1-dimethyl-1H-pyrazolo[4,3-c]quinoline-8-carboxamide

To a mixture of 4-amino-1-methyl-1H-pyrazolo[4,3-c]quinoline-8-carboxylic acid (945 mg, 3.9 mmol, 1.0 eq.), (S)-6-bromo-N-methyl-2,3-dihydrobenzofuran-3-amine (890 mg, 3.9 mmol, 1.0 eq.), DIEA (1.51 g, 11.7 mmol, 3.0 eq.) in DMF (10 mL) was added HATU (2.23 g, 5.85 mmol, 1.5 eq.) at 0° C. The reaction mixture was stirred for 1 hour under N₂ atmosphere. The mixture was purified by flash chromatographer (eluent, 0~3% MeOH in DCM) to give(S)-4-amino-N-(6-bromo-2,3-dihydrobenzofuran-3-yl)-N, 1-dimethyl-1H-pyrazolo[4,3-c]quinoline-8-carboxamide (1.3 g, 74% yield) as a yellow solid.

LC-MS (ESI): mass calcd, for C₂₁H₁₈BrN₅O₂, 451/453; m/z found, 451.6/453.6 [M+H]⁺.

Step B: tert-butyl(S)-(8-(6-bromo-2,3-dihydrobenzofuran-3-yl)(methyl)carbamoyl)-1-methyl-1H-pyrazolo[4,3-c]quinolin-4-yl)(tert-butoxycarbonyl)carbamate

To a mixture of(S)-4-amino-N-(6-bromo-2,3-dihydrobenzofuran-3-yl)-N,1-dimethyl-1H-pyrazolo[4,3-c]quinoline-8-carboxamide (1.3 g, 2.9 mmol, 1.0 eq.), Boc₂O (1.42 g, 6.5 mmol, 2.1 eq.), DIEA (800 mg, 6.19 mmol, 2.0 eq.) in DCM (13 mL) was added DMAP (76 mg, 619 μmol, 0.2 eq.) at 0° C. The reaction mixture was stirred for 2 hours under N₂ atmosphere. The mixture was purified by flash chromatographer (eluent, 0~35% EtOAc in PE) to give tert-butyl(S)-(8-((6-bromo-2,3-dihydrobenzofuran-3-yl)(methyl)carbamoyl)-1-methyl-1H-pyrazolo[4,3-c]quinolin-4-yl)(tert-butoxycarbonyl)carbamate (1.8 g, 96% yield) as a white solid.

LC-MS (ESI): mass calcd, for C₃₁H₃₄BrN₅O₆, 651/653; m/z found, 652.3/654.3 [M+H]⁺.

Step C: methyl(S)-5-(3-(4-(bis(tert-butoxycarbonyl)amino)-N, 1-dimethyl-1H-pyrazolo[4,3-c]quinoline-8-carboxamido)-2,3-dihydrobenzofuran-6-yl)picolinate

To a mixture of tert-butyl(S)-(8-((6-bromo-2,3-dihydrobenzofuran-3-yl)(methyl)carbamoyl)-1-methyl-1H-pyrazolo[4,3-c]quinolin-4-yl)(tert-butoxycarbonyl)carbamate (1.3 g, 1.99 mmol, 1 eq.), methyl 5-(4,4,5,5-tetramethyl-1,3,2-dioxaborolan-2-yl)picolinate (629 mg, 2.39 mmol, 1.2 eq.) and K₂CO₃ (826 mg, 5.98 mol, 3.0 eq.) in 1,4-dioxane (20.0 mL) and H₂O (1.0 mL) was added Pd(dppf)Cl₂ (146 mg, 199 μmol, 0.1 eq.) under nitrogen atmosphere. Then the mixture was stirred at 90° C. for 3 hours. The cooled mixture was diluted with EtOAc and washed with H₂O and brine. The organic layers were dried over anhydrous Na₂SO₄, filtered and concentrated under reduced pressure to dryness. The residue was purified by flash chromatographer (eluent, 0~55% EtOAc in PE) to give the methyl(S)-5-(3-(4-(bis(tert-butoxycarbonyl)amino)-N, 1-dimethyl-1H-pyrazolo[4,3-c]quinoline-8-carboxamido)-2,3-dihydrobenzofuran-6-yl)picolinate (850 mg, 60% yield) as a white solid.

LC-MS (ESI): mass calcd, for C₃₈H₄₀N₆O₈, 708; m/z found, 709.4 [M+H]⁺.

Step D: (S)-5-(3-(4-(bis(tert-butoxycarbonyl)amino)-N, 1-dimethyl-1H-pyrazolo[4,3-c]quinoline-8-carboxamido)-2,3-dihydrobenzofuran-6-yl)picolinic acid

To a mixture of methyl(S)-5-(3-(4-(bis(tert-butoxycarbonyl)amino)-N,1-dimethyl-1H-pyrazolo[4,3-c]quinoline-8-carboxamido)-2,3-dihydrobenzofuran-6-yl)picolinate (850 mg, 1.2 mol, 1.0 eq.) in THF (4.0 mL) was added H₂O (1.0 mL) and LiOH·H₂O (43 mg, 1.8 mmol, 1.5 eq.) at room temperature. Then the mixture was stirred at room temperature for 2 hours. The mixture was concentrated under reduced pressure to dryness to give the(S)-5-(3-(4-(bis(tert-butoxycarbonyl)amino)-N, 1-dimethyl-1H-pyrazolo[4,3-c]quinoline-8-carboxamido)-2,3-dihydrobenzofuran-6-yl)picolinic acid (85 mg, 88% yield) as a brown solid.

LC-MS (ESI): mass calcd, for C₃₇H₃₈N₆O₈, 694; m/z found, 694.8 [M+H]⁺.

### Intermediate 2. (S)-5-(3-(4-amino-N-methyl-1,3-dihydrofuro[3,4-c][1,7]naphthyridine-8-carboxamido)-2,3-dihydrobenzofuran-6-yl)picolinic acid

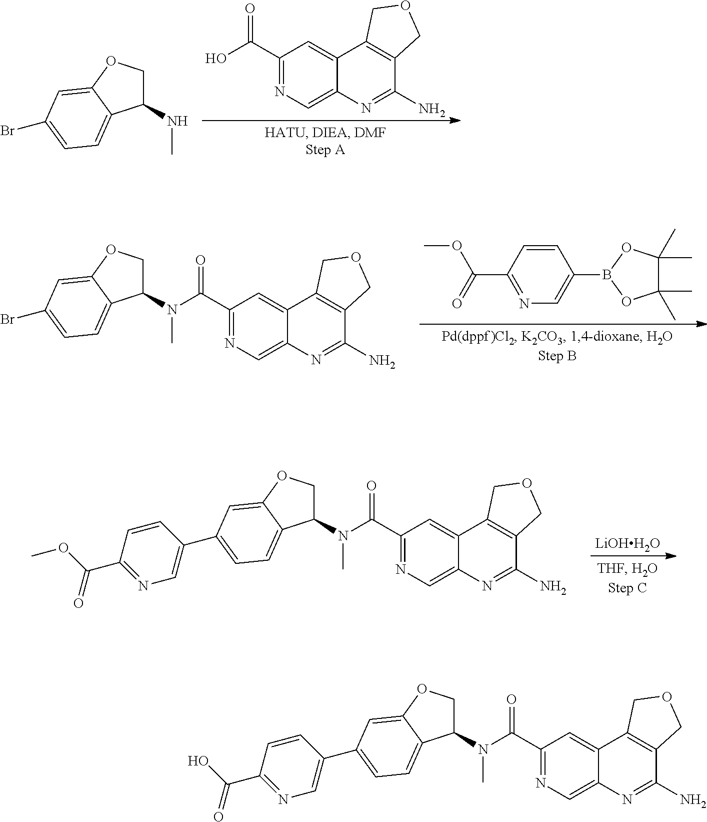

Step A: (S)-4-amino-N-(6-bromo-2,3-dihydrobenzofuran-3-yl)-N-methyl-1,3-dihydrofuro[3,4-c][1,7]naphthyridine-8-carboxamide

To a mixture of 4-amino-1,3-dihydrofuro[3,4-c][1,7]naphthyridine-8-carboxylic acid (100 mg, 433 μmol, 1.0 eq.), (S)-6-bromo-N-methyl-2,3-dihydrobenzofuran-3-amine (99 mg, 433 μmol, 1.0 eq.), DIEA (168 mg, 1.30 mmol, 3.0 eq.) in DMF (1 mL) was added HATU (247 mg, 649 μmol, 1.5 eq.) at 0° C. The reaction mixture was stirred for 1 hour under N₂ atmosphere. The mixture was purified by flash chromatographer (eluent, 0~3% MeOH in DCM) to give(S)-4-amino-N-(6-bromo-2,3-dihydrobenzofuran-3-yl)-N-methyl-1,3-dihydrofuro[3,4-c][1,7]naphthyridine-8-carboxamide (130 mg, 68% yield) as a yellow solid.

LC-MS (ESI): mass calcd, for C₂₀H₁₇BrN₄O₃, 440/442; m/z found, 440.6/442.6 [M+H]⁺.

Step B: methyl(S)-5-(3-(4-amino-N-methyl-1,3-dihydrofuro[3,4-c][1,7]naphthyridine-8-carboxamido)-2,3-dihydrobenzofuran-6-yl)picolinate

To a mixture of(S)-4-amino-N-(6-bromo-2,3-dihydrobenzofuran-3-yl)-N-methyl-1,3-dihydrofuro[3,4-c][1,7]naphthyridine-8-carboxamide (120 mg, 272 μmol, 1.0 eq.), methyl 5-(4,4,5,5-tetramethyl-1,3,2-dioxaborolan-2-yl)picolinate (86 mg, 326 μmol, 1.2 eq.) and K₂CO₃ (113 mg, 816 μmol, 3.0 eq.) in 1,4-dioxane (2.0 mL) and H₂O (0.1 mL) was added Pd(dppf)Cl₂ (20 mg, 27.2 μmol, 0.1 eq.) under nitrogen atmosphere. Then the mixture was stirred at 90° C. for 3 hours. The cooled mixture was diluted with EtOAc and washed with H₂O and brine. The organic layers were dried over anhydrous Na₂SO₄, filtered and concentrated under reduced pressure to dryness. The residue was purified by flash chromatographer (eluent, 0~3% MeOH in DCM) to give the methyl(S)-5-(3-(4-amino-N-methyl-1,3-dihydrofuro[3,4-c][1,7]naphthyridine-8-carboxamido)-2,3-dihydrobenzofuran-6-yl)picolinate (110 mg, 81% yield) as a brown solid.

LC-MS (ESI): mass calcd, for C₂₇H₂₃N₅O₅, 497; m/z found, 498.2 [M+H]⁺.

Step C: (S)-5-(3-(4-amino-N-methyl-1,3-dihydrofuro[3,4-c][1,7]naphthyridine-8-carboxamido)-2,3-dihydrobenzofuran-6-yl)picolinic acid

To a mixture of methyl(S)-5-(3-(4-amino-N-methyl-1,3-dihydrofuro[3,4-c][1,7]naphthyridine-8-carboxamido)-2,3-dihydrobenzofuran-6-yl)picolinate (100 mg, 201 μmol, 1.0 eq.) in THF (2.0 mL) was added H₂O (0.4 mL) and LiOH·H₂O (7.2 mg, 302 μmol, 1.5 eq.) at room temperature. Then the mixture was stirred at room temperature for 2 hours. The mixture was concentrated under reduced pressure to dryness to give the(S)-5-(3-(4-amino-N-methyl-1,3-dihydrofuro[3,4-c][1,7]naphthyridine-8-carboxamido)-2,3-dihydrobenzofuran-6-yl)picolinic acid (85 mg, 88% yield) as a brown solid.

LC-MS (ESI): mass calcd, for C₂₆H₂₁N₅O₅, 483; m/z found, 484.2 [M+H]⁺.

### Intermediate 3. (S)-5-(3-(2-amino-N,3-dimethylquinoline-6-carboxamido)-2,3-dihydrobenzofuran-6-yl)picolinic acid

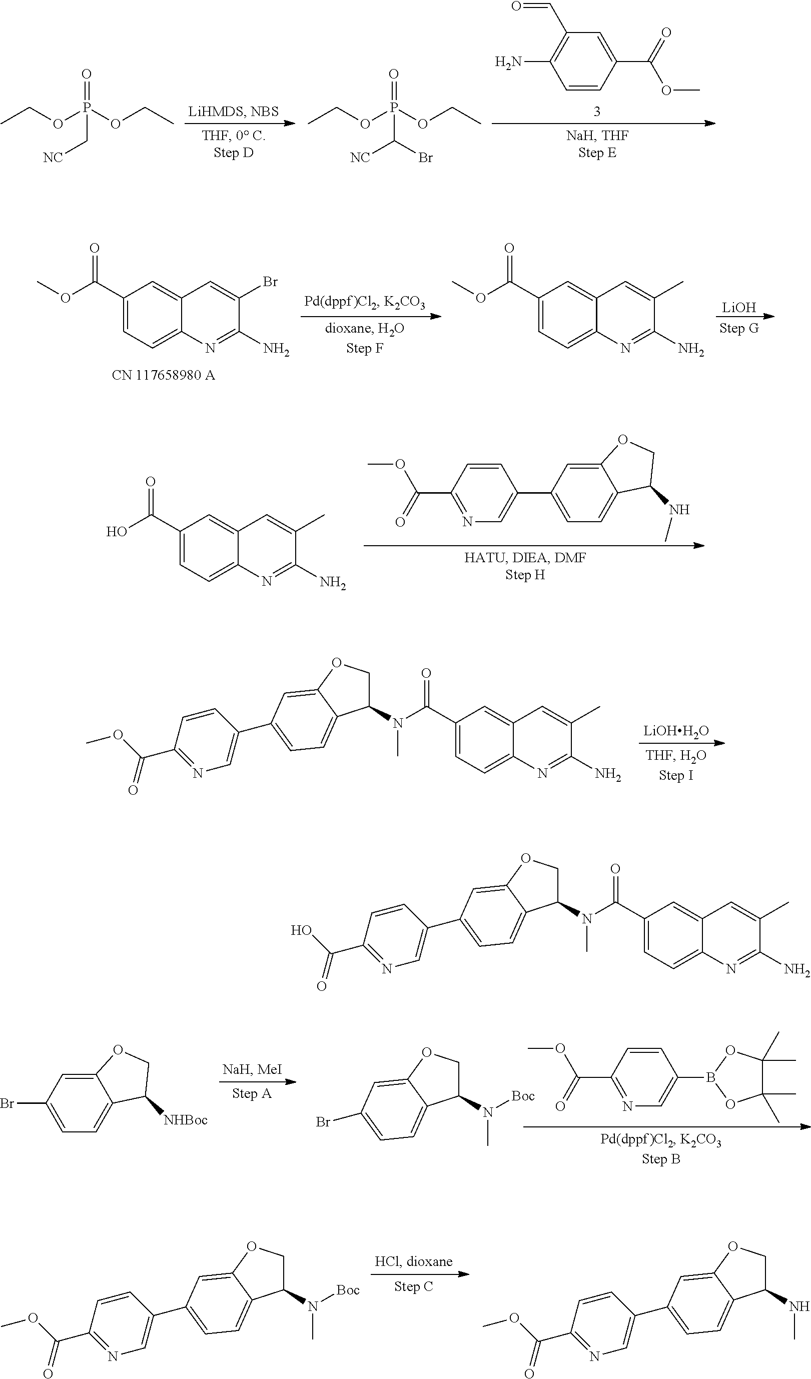

Step A: tert-butyl(S)-(6-bromo-2,3-dihydrobenzofuran-3-yl)(methyl)carbamate

A solution of tert-butyl(S)-(6-bromo-2,3-dihydrobenzofuran-3-yl)carbamate (3.0 g, 9.6 mmol, 1.0 eq.) in DMF (30.0 mL) was added NaH (573 mg, 14.3 mmol, 1.5 eq.) and stirred for 30 min at 0° C. Then the Mel (896 μL, 14.3 mmol, 1.5 eq.) was added and the mixture was stirred at room temperature for 50 min. The resulting solution was diluted with 100 mL of water and extracted with EtOAc (30 mL×3). The combined organic layers were washed with brine (100 mL), dried over Na₂SO₄ and concentrated under reduced pressure. The residue was purified by flash column chromatography on silica gel (100-200 mesh silica gel, 0-5% EtOAc in PE) to give tert-butyl(S)-(6-bromo-2,3-dihydrobenzofuran-3-yl)(methyl)carbamate (3.0 g, 90% yield) as a yellow oil.

LC-MS (ESI): mass calcd, for C₁₄H₁₈BrNO₃, 327.05; m/z found, 274.2 [M−56+H]⁺.

Step B: methyl(S)-5-(3-((tert-butoxycarbonyl)(methyl)amino)-2,3-dihydrobenzofuran-6-yl)picolinate

To a mixture of tert-butyl(S)-(6-bromo-2,3-dihydrobenzofuran-3-yl)(methyl)carbamate (900 mg, 2.7 mmol, 1.0 eq.), methyl 5-(4,4,5,5-tetramethyl-1,3,2-dioxaborolan-2-yl)picolinate (938 mg, 3.6 mmol, 1.3 eq.), K₂CO₃ (1.5 g, 11.0 mmol, 4.0 eq.) and Pd(dppf)Cl₂ (201 mg, 274 μmol, 0.1 eq.) in 1,4-dioxane (20.0 mL) and Water (2.0 mL) was stirred at 90° C. under N₂ for 3 hours. The resulting solution was diluted with 100 mL of water and extracted with EtOAc (50 mL×3). The combined organic layers were washed with brine (100 mL.), dried over Na₂SO₄ and concentrated under reduced pressure. The residue was purified by flash column chromatography on silica gel (100-200 mesh silica gel, 0-25% EtOAc in PE) to give methyl(S)-5-(3-((tert-butoxycarbonyl)(methyl)amino)-2,3-dihydrobenzofuran-6-yl)picolinate (1.0 g, 95% yield) as a yellow solid.

LC-MS (ESI): mass calcd, for C₂₁H₂₄N₂O₅, 384.16; m/z found, 385.0 [M+H]⁺.

Step C: methyl(S)-5-(3-(methylamino)-2,3-dihydrobenzofuran-6-yl)picolinate

To a flask containing methyl(S)-5-(3-((tert-butoxycarbonyl)(methyl)amino)-2,3-dihydrobenzofuran-6-yl)picolinate (400 mg, 1.04 mmol, 1.0 eq.) in 1,4-Dioxane (5 mL) was added HCl (5 mL, 4 M in 1,4-dioxane) at room temperature. The reaction was stirred at room temperature for 2 hours. The residue was concentrated under reduced pressure to give methyl(S)-5-(3-(methylamino)-2,3-dihydrobenzofuran-6-yl)picolinate (290 mg, 98% yield) as a yellow solid.

LC-MS (ESI): mass calcd, for C₁₆H₁₆N₂O₃, 284.12; m/z found, 285.3 [M+H]⁺.

Step D: diethyl (bromo (cyano)methyl)phosphonate

To a solution of methyl 2-amino-3-bromoquinoline-6-carboxylate (10.0 g, 56.5 mmol, 1.0 eq.) in THF (150 mL) was added dropwise LiHMDS (68 mL, 68 mmol, 1.2 eq., 1 M in THF) at −60° C. The resulting mixture was stirred at −60° C. under N₂ for 0.5 hour. Then NBS (9.0 g, 50.5 mmol, 0.9 eq.) in THF (100 mL) was added dropwise to the reaction under N₂ at −60° C. The resulting mixture was stirred at −60° C. under N₂ for 3 hours. The reaction mixture was poured into water (100 mL) and then extracted with EtOAc (100 mL×2). The combined organic layers were washed with brine, dried over anhydrous Na₂SO₄ and concentrated. The residue was purified by column chromatography (silica gel, 0~30% EtOAc in PE) to afford diethyl (bromo (cyano)methyl)phosphonate (9.2 g, 63% yield) as a colorless oil.

LC-MS (ESI): mass calcd, for C₆H₁₁BrNO₃P, 216.0; m/z found, 256.0 [M+H]⁺.

Step E: methyl 2-amino-3-bromoquinoline-6-carboxylate

To a solution of methyl 4-amino-3-formylbenzoate (5.0 g, 35.2 mmol, 1.0 eq.) in THF (100 mL) was added NaH (1.6 g, 42 mmol, 1.2 eq.) at 0° C. under N₂. The resulting mixture was stirred at 0° C. under N₂ for 0.5 hour. Then diethyl (bromo (cyano)methyl)phosphonate (9.0 g, 35.2 mmol, 1.2 eq.) in THF (50 mL) was added dropwise to the reaction under N₂ at 0° C. The resulting mixture was stirred at room temperature under N₂ for 3 hours. The reaction mixture was poured into water (100 mL) and then extracted with EtOAc (100 mL×2). The combined organic layers were washed with brine, dried over anhydrous Na₂SO₄ and concentrated. The residue was purified by column chromatography (silica gel, 0~50% EtOAc in PE) to afford methyl 2-amino-3-bromoquinoline-6-carboxylate (4.6 g, 46% yield) as brown solid.

LC-MS (ESI): mass calcd, for C₆H₁₁BrNO₃P, 282.1; m/z found, 282/284 [M+H]⁺.

Step F: methyl 2-amino-3-methylquinoline-6-carboxylate

To a stirred mixture of methyl 2-amino-3-bromoquinoline-6-carboxylate (CN 117658980 A, 1.50 g, 5.34 mmol, 1.0 eq.) in dioxane (15 mL) and H₂O (1.5 mL) were added 2,4,6-trimethyl-1,3,5,2,4,6-trioxatriborinane (3.35 g, 7.6 mL, 26.7 mmol, 5.0 eq.), sodium carbonate (1.70 g, 16.0 mmol, 3.0 eq.) and Pd(dppf)Cl₂ (195 mg, 267 μmol, 0.05 eq.). The resulting mixture was stirred at 100° C. under N₂ for 15 hours. The reaction mixture was poured into water (100 mL) and then extracted with EtOAc (100 mL×2). The combined organic layers were washed with brine, dried over anhydrous Na₂SO₄ and concentrated. The residue was purified by column chromatography (silica gel, PE~100% EtOAc in PE) to afford methyl 2-amino-3-methylquinoline-6-carboxylate (790 mg, 69% yield) as a white solid.

LC-MS (ESI): mass calcd, for C₁₂H₁₂N₂O₂, 216.09; m/z found, 216.9 [M+H]⁺.

Step G: 2-amino-3-methylquinoline-6-carboxylic acid

To a stirred mixture of methyl 2-amino-3-methylquinoline-6-carboxylate (790 mg, 3.65 mmol, 1.0 eq.) in THF (8 mL)/H₂O (4 mL) was added LiOH (307 mg, 7.31 mmol, 2.0 eq.). The resulting mixture was stirred at room temperature for 15 hours. The mixture was concentrated under reduced pressure to give 2-amino-3-methylquinoline-6-carboxylic acid (700 mg, 95% yield) as a white solid.

LC-MS (ESI): mass calcd, for C₁₁H₁₀N₂O₂, 202.07; m/z found, 202.9 [M+H]⁺.

Step H: methyl(S)-5-(3-(2-amino-N,3-dimethylquinoline-6-carboxamido)-2,3-dihydrobenzofuran-6-yl)picolinate

To a stirred mixture of 2-amino-3-methylquinoline-6-carboxylic acid (150 mg, 742 μmol, 1.0 eq.) in DMF (6 mL) were added methyl(S)-5-(3-(methylamino)-2,3-dihydrobenzofuran-6-yl)picolinate (211 mg, 742 μmol, 1.0 eq.), HATU (423 mg, 1.11 mmol, 1.5 eq.) and DIEA (646 μL, 3.71 mmol, 5.0 eq.). The mixture was stirred at room temperature for 2 hours. The reaction mixture was poured into water (60 mL) and then extracted with EtOAc (50 mL×2). The combined organic layers were washed with brine, dried over anhydrous Na₂SO₄ and concentrated. The crude product was purified by flash silica chromatography, eluting with a gradient of 0~8% MeOH in DCM to afford methyl(S)-5-(3-(2-amino-N,3-dimethylquinoline-6-carboxamido)-2,3-dihydrobenzofuran-6-yl)picolinate (110 mg, 32% yield) as a yellow solid.

LC-MS (ESI): mass calcd, for C₂₇H₂₄N₄O₄, 468.18; m/z found, 469.2 [M+H]⁺.

Step I: (S)-5-(3-(2-amino-N,3-dimethylquinoline-6-carboxamido)-2,3-dihydrobenzofuran-6-yl)picolinic acid

A solution of methyl(S)-5-(3-(2-amino-N,3-dimethylquinoline-6-carboxamido)-2,3-dihydrobenzofuran-6-yl)picolinate (110 mg, 235 μmol, 1.0 eq.) and LiOH (11 mg, 470 μmol, 2.0 eq.) in THE (3 mL) and Water (1.5 mL) with stirring at room temperature. The mixture was stirred at room temperature for 2 hours. The residue was concentrated under reduced pressure to give(S)-5-(3-(2-amino-N,3-dimethylquinoline-6-carboxamido)-2,3-dihydrobenzofuran-6-yl) picolinic acid (100 mg, 94% yield) as a solid.

LC-MS (ESI): mass calcd, for C₂₆H₂₂N₄O₄, 454.16; m/z found, 455.2 [M+H]⁺.

### Intermediate 4. (S)-5-(3-(2-amino-3-cyclopropyl-N-methylquinoline-6-carboxamido)-2,3-dihydrobenzofuran-6-yl)picolinic acid

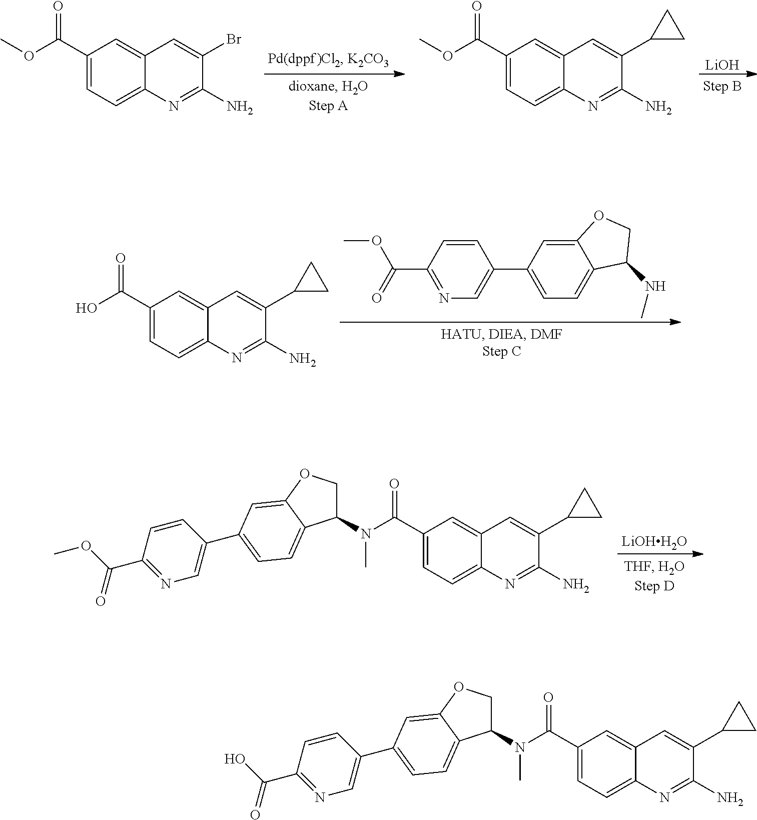

Step A: methyl 2-amino-3-cyclopropylquinoline-6-carboxylate

To a stirred mixture of methyl 2-amino-3-bromoquinoline-6-carboxylate (1.0 g, 3.5 mmol, 1.0 eq.) in dioxane (15 mL) and H₂O (1.5 mL) were added methyl 2-amino-3-cyclopropylquinoline-6-carboxylate (600 mg, 7.0 mmol, 2.0 eq.), potassium phosphate tribasic (2.2 g, 10.5 mmol, 3.0 eq.), tricyclohexyl phosphine (100 mg, 350 μmol, 0.1 eq.) and Pd (OAc) 2 (70 mg, 350 μmol, 0.1 eq.). The resulting mixture was stirred at 100° C. under N₂ for 15 hours. The reaction mixture was poured into water (100 mL) and then extracted with EtOAc (100 mL×2). The combined organic layers were washed with brine, dried over anhydrous Na₂SO₄ and concentrated. The residue was purified by column chromatography (silica gel, 0~50% EtOAc in PE) to afford methyl 2-amino-3-cyclopropylquinoline-6-carboxylate (420 mg, 48% yield) as a yellow solid.

LC-MS (ESI): mass calcd, for C₁₂H₁₂N₂O₂, 242.3; m/z found, 243.3 [M+H]⁺.

Step B: 2-amino-3-cyclopropylquinoline-6-carboxylic acid

To a stirred mixture of methyl 2-amino-3-cyclopropylquinoline-6-carboxylate (283 mg, 1.17 mmol, 1.0 eq.) in THF (4 mL) and H₂O (2 mL) were added LiOH (98 mg, 2.34 mmol, 2.0 eq.). The resulting mixture was stirred at room temperature for 15 hours. The pH of the mixture was adjusted to around pH=7 by progressively adding 1 mol/L aq. HCl solution. The mixture was filtered, and the filter cake was concentrated to give 2-amino-3-cyclopropylquinoline-6-carboxylic acid (228 mg, 86% yield) as a white solid.

LC-MS (ESI): mass calcd, for C₁₃H₁₂N₂O₂, 228.09; m/z found, 229.2 [M+H]⁺.

Step C: methyl(S)-5-(3-(2-amino-3-cyclopropyl-N-methylquinoline-6-carboxamido)-2,3-dihydrobenzofuran-6-yl)picolinate

To a mixture of methyl(S)-5-(3-(methylamino)-2,3-dihydrobenzofuran-6-yl)picolinate (148 mg, 521 μmol, 1.0 eq.), HATU (396 mg, 1.04 mmol, 2.0 eq.) and 2-amino-3-cyclopropylquinoline-6-carboxylic acid (143 mg, 625 μmol, 1.2 eq.) in DMF (2 mL) was added DIEA (363 μL, 2.08 mmol, 4.0 eq.). The reaction mixture was purged with nitrogen three times, then stirred at room temperature for 30 min under nitrogen. The reaction mixture was poured into water (100 mL) and then extracted with EtOAc (100 mL×2). The combined organic layers were washed with brine, dried over anhydrous Na₂SO₄ and concentrated. The crude product was purified by flash silica chromatography, elution with a gradient of 0 to 100% EtOAc in PE. The desired fractions were evaporated to dryness to afford methyl(S)-5-(3-(2-amino-3-cyclopropyl-N-methylquinoline-6-carboxamido)-2,3-dihydrobenzofuran-6-yl)picolinate (180 mg, 70% yield) as a yellow solid.

LC-MS (ESI): mass calcd, for C₂₉H₂₆N₄O₄,494.20; m/z found, 495.3 [M+H]⁺.

Step D: (S)-5-(3-(2-amino-3-cyclopropyl-N-methylquinoline-6-carboxamido)-2,3-dihydrobenzofuran-6-yl)picolinic acid

To a solution of methyl(S)-5-(3-(2-amino-3-cyclopropyl-N-methylquinoline-6-carboxamido)-2,3-dihydrobenzofuran-6-yl)picolinate (180 mg, 364 μmol, 1.0 eq.) in THF (2 mL) and Water (1 mL) was added LiOH (17.4 mg, 728 μmol, 2.0 eq.). The reaction mixture was stirred at room temperature for 1 hour under nitrogen. The reaction mixture was evaporated to dryness to afford(S)-5-(3-(2-amino-3-cyclopropyl-N-methylquinoline-6-carboxamido)-2,3-dihydrobenzofuran-6-yl)picolinic acid (170 mg, 97% yield) as a white solid.

LC-MS (ESI): mass calcd, for C₂₈H₂₄N₄O₄, 480.18; m/z found, 481.3 [M+H]⁺.

### Intermediate 5. N-((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl) phenoxy)ethoxy)cyclohexyl)-5-((S)-3-(methylamino)-2,3-dihydrobenzofuran-6-yl)picolinamide

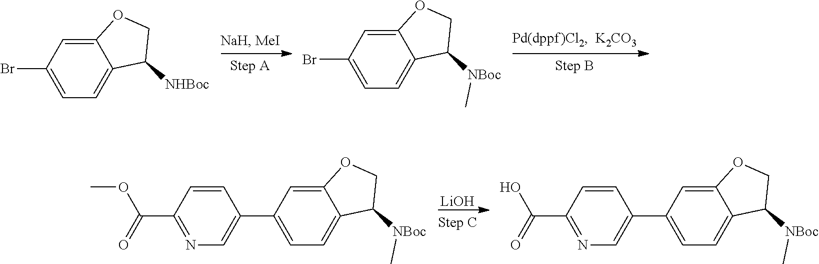

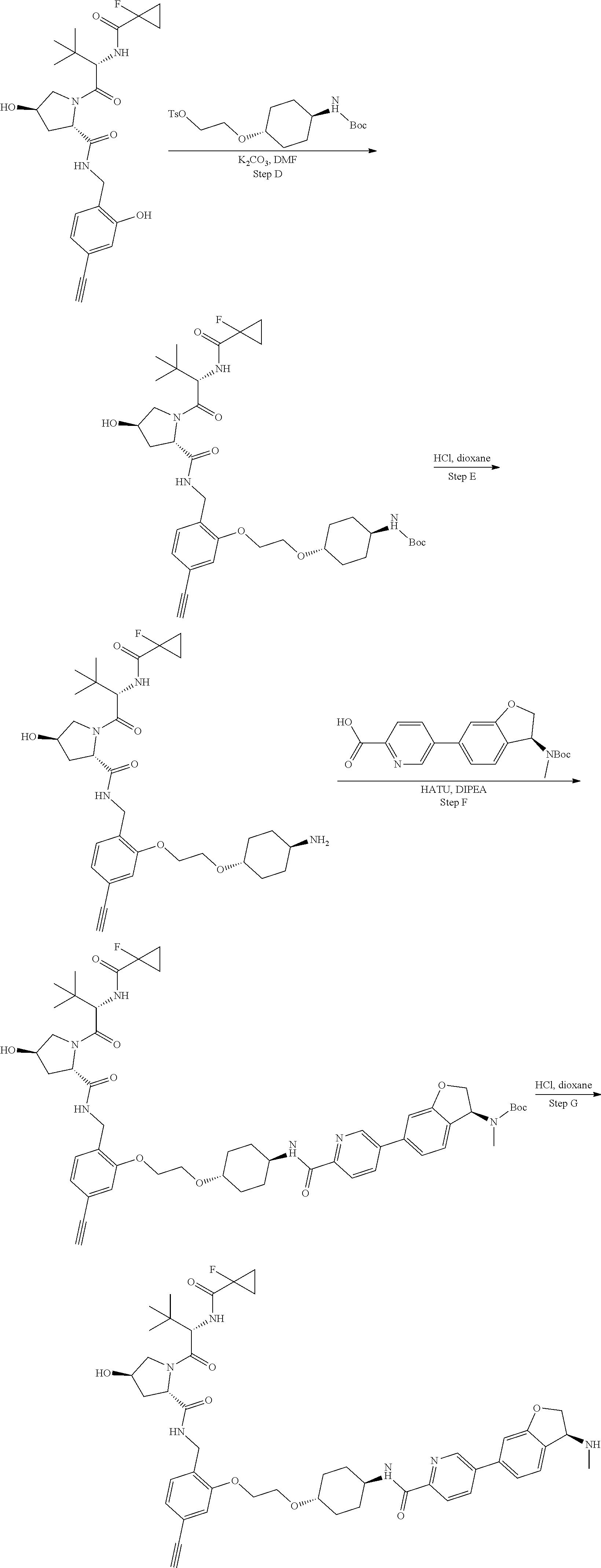

Step A: tert-butyl(S)-(6-bromo-2,3-dihydrobenzofuran-3-yl)(methyl)carbamate

A solution of tert-butyl(S)-(6-bromo-2,3-dihydrobenzofuran-3-yl)carbamate (3.0 g, 9.6 mmol, 1.0 eq.) in DMF (30.0 mL) was added NaH (573 mg, 14.3 mmol, 1.5 eq.) and stirred for 30 min at 0° C. Then the Mel (896 μL, 14.3 mmol, 1.5 eq.) was added and the mixture was stirred at room temperature for 1 hour. The resulting solution was diluted with 100 ml of water and extracted with EtOAc (30 mL×3). The combined organic layers were washed with brine (100 mL), dried over Na₂SO₄ and concentrated under reduced pressure. The residue was purified by flash column chromatography on silica gel (100-200 mesh silica gel, 0-5% EtOAc in PE) to give tert-butyl(S)-(6-bromo-2,3-dihydrobenzofuran-3-yl)(methyl)carbamate (3.0 g, 90% yield) as a yellow oil.

LC-MS (ESI): mass calcd, for C₁₄H₁₈BrNO₃, 327.05; m/z found, 274.2 [M−56+H]⁺.

Step B: methyl(S)-5-(3-((tert-butoxycarbonyl)(methyl)amino)-2,3-dihydrobenzofuran-6-yl)picolinate

To a mixture of tert-butyl(S)-(6-bromo-2,3-dihydrobenzofuran-3-yl)(methyl)carbamate (900 mg, 2.7 mmol, 1.0 eq.), methyl 5-(4,4,5,5-tetramethyl-1,3,2-dioxaborolan-2-yl)picolinate (938 mg, 3.6 mmol, 1.3 eq.), K₂CO₃ (1.5 g, 11.0 mmol, 4.0 eq.) and PdCl₂ (dppf)(201 mg, 274 μmol, 0.1 eq.) in 1,4-dioxane (20.0 mL) and Water (2.0 mL) was stirred at 90° C. under N₂ for 3 hours. The resulting solution was diluted with 100 ml of water and extracted with EtOAc (50 mL×3). The combined organic layers were washed with brine (100 mL), dried over Na₂SO₄ and concentrated under reduced pressure. The residue was purified by flash column chromatography on silica gel (100-200 mesh silica gel, 0-25% EtOAc in PE) to give methyl(S)-5-(3-((tert-butoxycarbonyl)(methyl)amino)-2,3-dihydrobenzofuran-6-yl)picolinate (1.0 g, 95% yield) as a yellow solid.

LC-MS (ESI): mass calcd, for C₂₁H₂₄N₂O₅, 384.16; m/z found, 385.0 [M+H]⁺.

Step C: (S)-5-(3-((tert-butoxycarbonyl)(methyl)amino)-2,3-dihydrobenzofuran-6-yl)picolinic acid

To a solution of methyl(S)-5-(3-((tert-butoxycarbonyl)(methyl)amino)-2,3-dihydrobenzofuran-6-yl)picolinate (300 mg, 780 μmol, 1.0 eq.) in THF (6.0 mL) and Water (5.0 mL) was added Lithium hydroxide monohydrate (66 mg, 1.56 mmol, 2.0 eq.) and stirred at room temperature for 50 min. The resulting solution was concentrated under reduced pressure to give(S) -5-(3-((tert-butoxycarbonyl)(methyl)amino)-2,3-dihydrobenzofuran-6-yl)picolinic acid (280 mg, 97% yield) as a yellow solid.

LC-MS (ESI): mass calcd, for C₂₀H₂₂N₂O₅, 370.15; m/z found, 371.3 [M+H]⁺.

Step D: tert-butyl ((1S,4r)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl) phenoxy)ethoxy)cyclohexyl)carbamate

To a solution of (2S,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide (230 mg, 501 μmol, 1.0 eq.), 2-(((1r,4r)-4-((tert-butoxycarbonyl)amino)cyclohexyl)oxy)ethyl 4-methylbenzenesulfonate (310 mg, 751 μmol, 1.5 eq.) and KI (17 mg, 100 μmol, 0.2 eq.) in DMF (8.0 mL) was added K₂CO₃ (208 mg, 1.50 mmol, 3.0 eq.). The mixture was stirred at 75° C. for 6 hours. The resulting solution was diluted with 50 mL of water and extracted with EtOAc (30 mL×3). The combined organic layers were washed with brine (50 mL), dried over Na₂SO₄ and concentrated under reduced pressure. The residue was purified by flash column chromatography on silica gel (100-200 mesh silica gel, 0-5% MeOH in DCM) to give tert-butyl ((1S,4r)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamate (200 mg, 57% yield) as a yellow oil.

LC-MS (ESI): mass calcd, for C₃₇H₅₃FN₄O₈, 700.38; m/z found, 701.6 [M+H]⁺.

Step E: (2S,4R)—N-(2-(2-(((1r,4S)-4-aminocyclohexyl)oxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide

To a solution of tert-butyl ((1S,4r)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamate (200 mg, 285 μmol, 1.0 eq.) in DCM (6.0 mL) was added HCl-dioxane (2.0 mL) and stirred at room temperature for 1 hour. The resulting solution was concentrated under reduced pressure to give (2S,4R)—N-(2-(2-(((1r,4S)-4-aminocyclohexyl)oxy)ethoxy)-4-ethynylbenzyl)-1-((S)-2-(1-fluorocyclopropane-1-carboxamido)-3,3-dimethylbutanoyl)-4-hydroxypyrrolidi-ne-2-carboxamide (170 mg, 99% yield) as a yellow oil.

LC-MS (ESI): mass calcd, for C₃₂H₄₅FN₄O₆, 600.33; m/z found, 601.1 [M+H]⁺.

Step F: tert-butyl ((S)-6-(6-(((1S, 4S)-4-(2-(5-ethynyl-2-(((2S,4R)-1-((S)-2-(1-fluorocyclopropane-1-carboxamido) ~3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)(methyl)carbamate

To a solution of(S)-5-(3-((tert-butoxycarbonyl)(methyl)amino)-2,3-dihydrobenzofuran-6-yl)picolinic acid (80 mg, 216 μmol, 1.0 eq.), (2S,4R)—N-(2-(2-(((1r,4S)-4-aminocyclohexyl)oxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide (156 mg, 259 μmol, 1.0 eq.) and HATU (123 mg, 324 μmol, 1.5 eq.) in DMF (5.0 mL) was added DIEA (150 μL, 864 μmol, 4.0 eq.). The mixture was stirred at room temperature for 0.5 hour. The resulting solution was diluted with 30 mL of water and extracted with EtOAc (20 mL×3). The combined organic layers were washed with brine (30 mL), dried over Na₂SO₄ and concentrated under reduced pressure. The residue was purified by flash column chromatography on silica gel (100-200 mesh silica gel, 0-5% MeOH in DCM) to give tert-butyl ((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)(methyl)carbamate (120 mg, 58% yield) as a yellow oil.

LC-MS (ESI): mass calcd, for C₅₂H₆₅FN₆O₁₀, 952.47; m/z found, 953.3 [M+H]⁺.

Step G: N-((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)pheno-xy)ethoxy)cyclohexyl)-5-(S)-3-(methylamino)-2,3-dihydrobenzofuran-6-yl)picolinamide

To a solution of tert-butyl ((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzo-furan-3-yl)(methyl)carbamate (120 mg, 126 μmol, 1.0 eq.) in DCM (5.0 mL) was added HCl-dioxane (3.0 mL) and stirred at room temperature for 1 hour. The resulting solution was concentrated under reduced pressure to give N-((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)-5-((S)-3-(methylamino)-2,3-dihydrobenzo-furan-6-yl)picolinamide (100 mg, 93% yield) as a yellow solid.

LC-MS (ESI): mass calcd, for C₄₇H₅₇FN₆O₈, 852.42; m/z found, 853.8 [M+H]⁺.

### Example A1. 4-amino-N—((S)-6-(6-((17-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)-3,6,9,12,15-pentaoxaheptadecyl)carbamoyl)pyridin-3-yl)-2,3-dihydrobenzofuran-3-yl)-N,1-dimethyl-1H-pyrazolo[4,3-c]quinoline-8-carboxamide

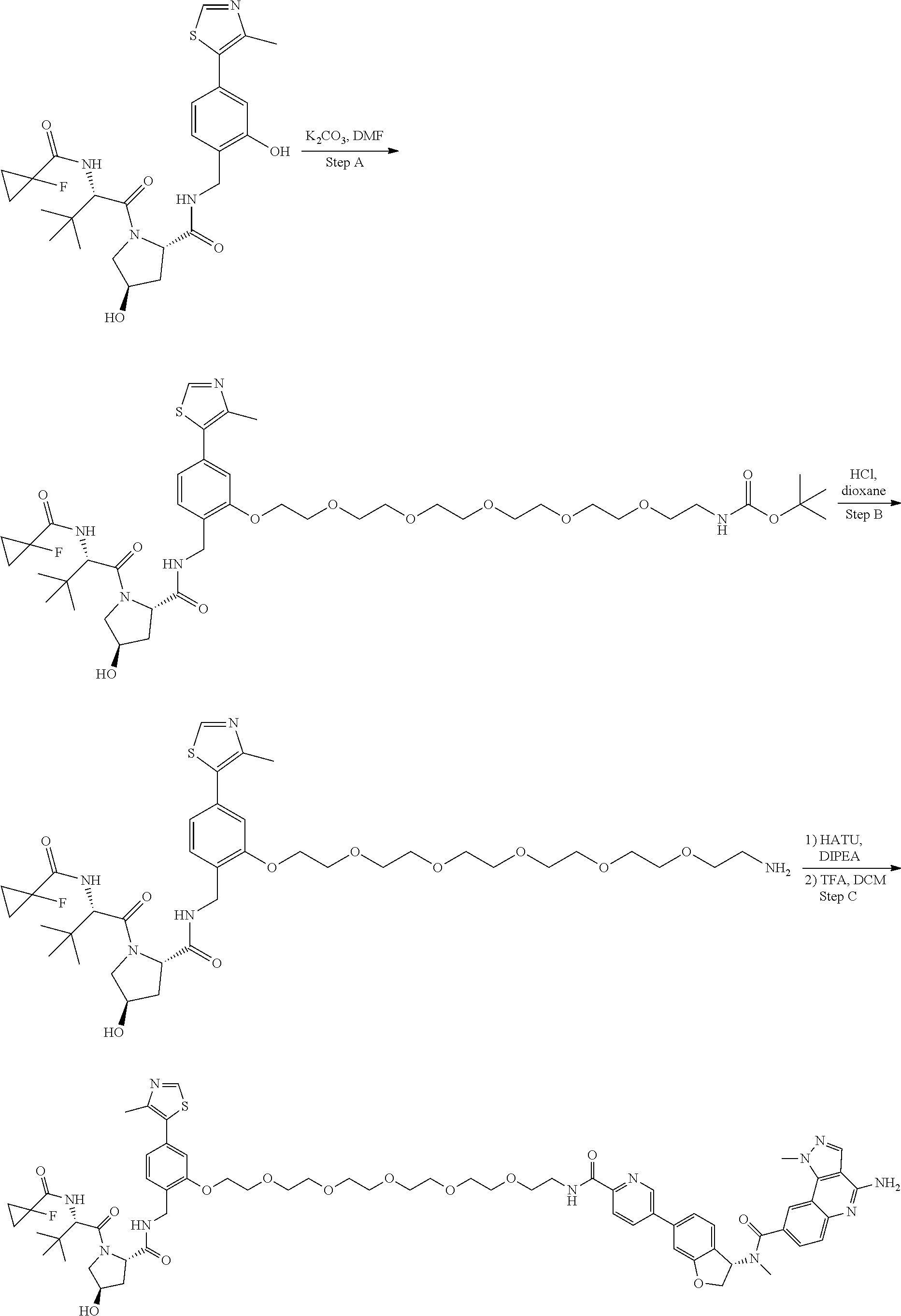 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCNC(=O)c2ccc(-c3ccc4c(c3)OC[C@H]4N(C)C(=O)c3ccc4nc(N)c5cnn(C)c5c4c3)cn2)c1]

Step A: tert-butyl (17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12, 15-pentaoxaheptadecyl)carbamate

To a solution of tert-butyl (17-bromo-3,6,9,12, 15-pentaoxaheptadecyl)carbamate (0.11 mg, 1.0 mmol, 1.0 eq.), (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (50 mg, 0.1 mmol, 1.0 eq.) in DMF (3 mL) was added K₂CO₃ (25 mg, 0.18 mmol, 3.0 eq.). The mixture was stirred at 60° C. for 16 hours under N₂ atmosphere. The mixture was diluted with water (30 mL) and extracted with DCM (20 mL×3). The combined organic layers were washed with brine, dried over anhydrous Na₂SO₄ and concentrated under reduced pressure. The residue was purified by flash chromatographer (eluent, 0~10% MeOH in DCM) to give tert-butyl (17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamate (55 mg, 65% yield) as a white solid.

LC-MS (ESI): mass calcd, for C₄₃H₆₆FN₅O₁₂S, 895.4; m/z found, 896.4 [M+H]⁺.

Step B: (2S,4R)—N-(2-((17-amino-3,6,9,12, 15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of tert-butyl (17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamate (55 mg, 0.6 mmol) in Dioxane (2 mL) was added HCl in Dioxane (2 mL, 4 mol/L) at 0° C. The reaction mixture was stirred for 1 hour at room temperature under N₂ atmosphere. The mixture was concentrated by vacuum to give (2S,4R)—N-(2-((17-amino-3,6,9,12,15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (35 mg, 71.6% yield) as a white solid.

LC-MS (ESI): mass calcd, for C₃₈H₅₈FN₅O₁₀S, 795.4; m/z found, 796.4 [M+H]⁺.

Step C: 4-amino-N—(S)-6-(6-((17-(2-(((2S,4R)-1-((S)-2-(1-fluorocyclopropane-1-carboxamido)-3, 3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamoyl)pyridin-3-yl)-2,3-dihydrobenzofuran-3-yl)-N,1-dimethyl-1H-pyrazolo[4,3-c]quinoline-8-carboxamide

To a solution of (28,4R)—N-(2-((17-amino-3,6,9,12, 15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (35 mg, 0.04 mmol, 1.0 eq.), (S)-5-(3-(4-(bis(tert-butoxycarbonyl)amino)-N, 1-dimethyl-1H-pyrazolo[4,3-c]quinoline-8-carboxamido)-2,3-dihydrobenzofuran-6-yl)picolinic acid (30 mg, 0.04 mmol, 1.0 eq.), HATU (40 mg, 0.1 mmol, 2.5 eq.) in DMF (2 mL) was added DIEA (50 mg, 0.4 mmol, 8.0 eq.) at 0° C. The reaction mixture was stirred at 0° C. for 30 mins under N₂ atmosphere. The mixture was diluted with water (30 mL) and extracted with EtOAc (20 mL×3). The combined organic layers were washed with brine, dried over anhydrous Na₂SO₄ and concentrated under reduced pressure. The residue was added DCM (0.5 mL) and TFA (0.5 mL), then stirred for 1 hour. The mixture was concentrated under reduced pressure. The residue was purified by prep-HPLC(C18, 20~95%, MeCN in H₂O with 0.1% HCOOH) to give 4-amino-N—((S)-6-(6-((17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12, 15-pentaoxaheptadecyl)carbamoyl)pyridin-3-yl)-2,3-dihydrobenzofuran-3-yl)-N, 1-dimethyl-1H-pyrazolo[4,3-c]quinoline-8-carboxamide (9.0 mg, 16% yield) as a white solid.

LC-MS (ESI): mass calcd, for C₆₃H₇₄FN₁₁O₁₂S, 1272.5; m/z found, 1273.5 [M+H]⁺.

### Example A2. 4-amino-N—((S)-6-(6-((14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12-tetraoxatetradecyl)carbam-oyl)pyridin-3-yl)-2,3-dihydrobenzofuran-3-yl)-N,1-dimethyl-1H-pyrazolo[4,3-c]quinoline-8-carboxamide

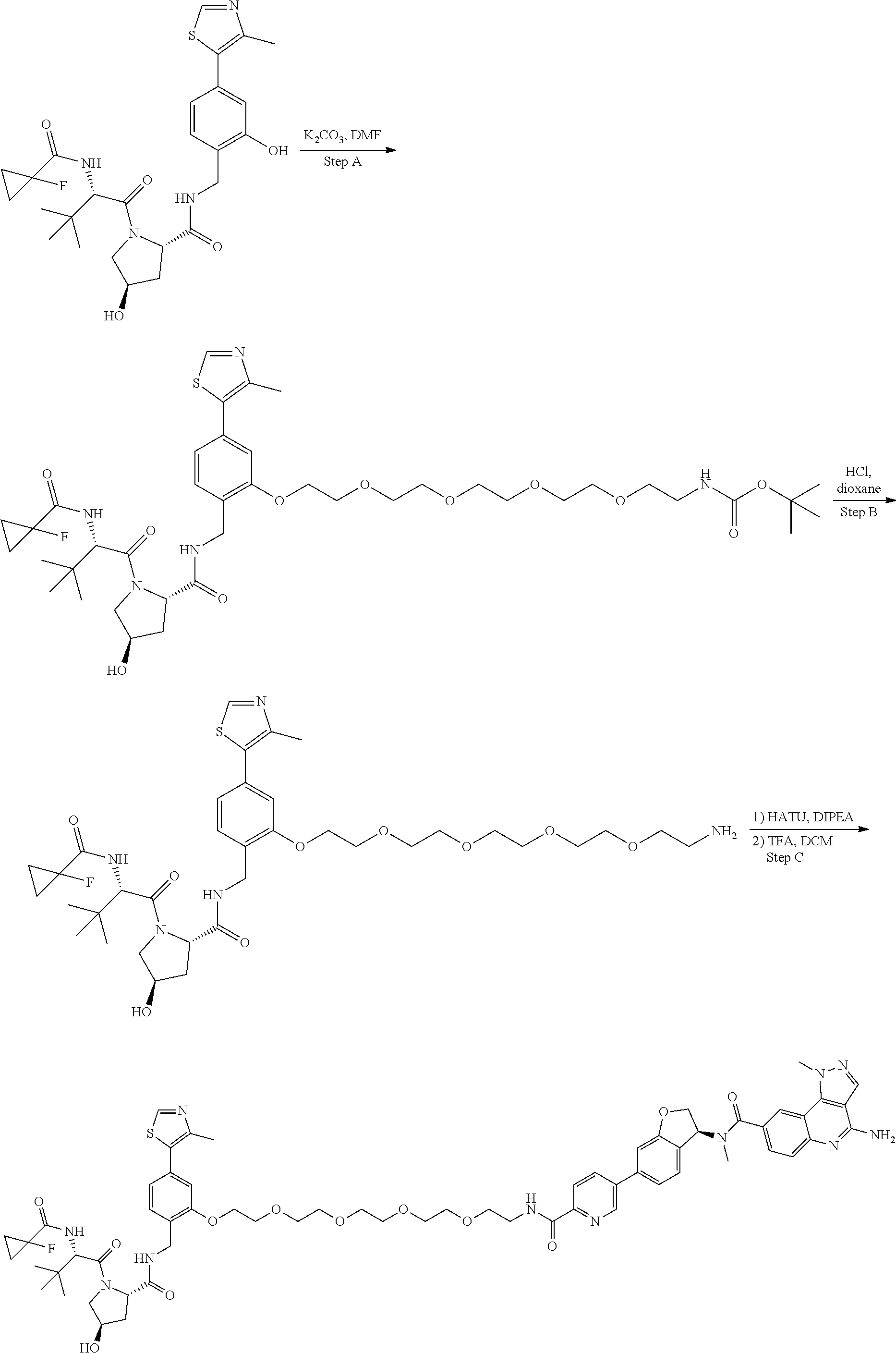 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)c2ccc(-c3ccc4c(c3)OC[C@H]4N(C)C(=O)c3ccc4nc(N)c5cnn(C)c5c4c3)cn2)c1]

Example A2 was synthesized following the same procedures as described in Example A1 (7.2 mg, 10% yield over 4 steps) as a white solid. LC-MS (ESI): mass calcd, for C₆₃H₇₄FN₁₁O₁₂S, 1227.5; m/z found, 1228.4 [M+H]⁺.

### Example A3. 4-amino-N—((S)-6-(6-((20-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)-3,6,9,12,15,18-hexaoxaicosyl)carbamoyl)pyridin-3-yl)-2,3-dihydrobenzofuran-3-yl)-N,1-dimethyl-1H-pyrazolo[4,3-c]quinoline-8-carboxamide

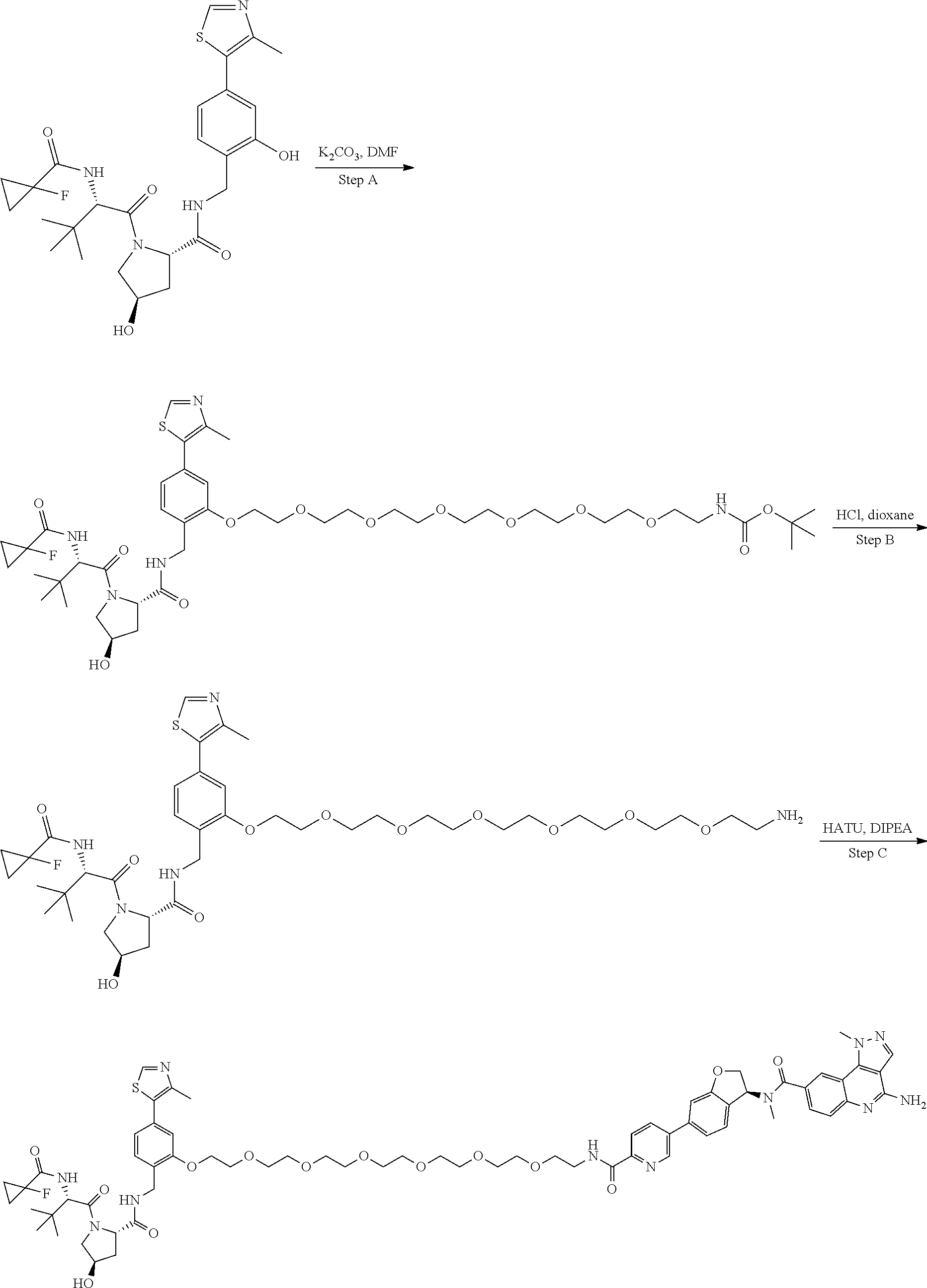 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCOCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCOCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCOCCNC(=O)c2ccc(-c3ccc4c(c3)OC[C@H]4N(C)C(=O)c3ccc4nc(N)c5cnn(C)c5c4c3)cn2)c1]

Example A3 was synthesized following the same procedures as described in Example A1 (0.5 mg, 3.7% yield over 3 steps) as a white solid. LC-MS (ESI): mass calcd. For C₆₇H₈₂FN₁₇O₁₄S, 1315.5; m/z found, 1316.4 [M+H]⁺.

### Example A4. 4-amino-N—((S)-6-(6-((2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)carbamoyl)pyridin-3-yl)-2,3-dihydrobenzofuran-3-yl)-N,1-dimethyl-1H-pyrazolo[4,3-c]quinoline-8-carboxamide

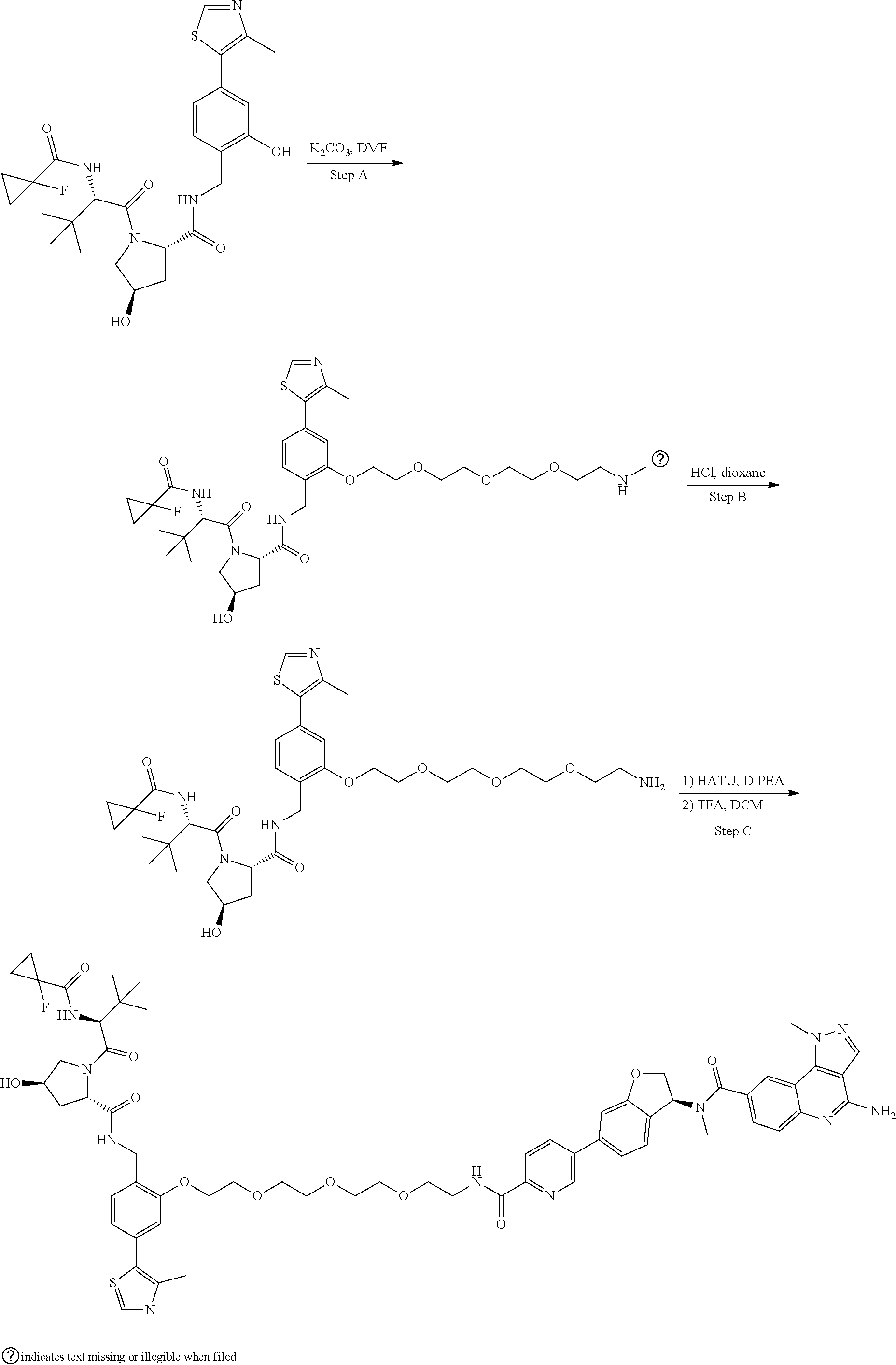

Example A4 was synthesized following the same procedures as described in Example A1 (5.0 mg, 58% yield over 3 steps) as a white solid. LC-MS (ESI): mass calcd, for C₆H₇FN₁₁O₁₁S, 1183.5; m/z found, 1184.6 [M+H]⁺.

### Example A5. 4-amino-N—((S)-6-(6-((11-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy) undecyl)carbamoyl)pyridin-3-yl)-2,3-dihydrobenzofuran-3-yl)-N,1-dimethyl-1H-pyrazolo[4,3-c]quinoline-8-carboxamide

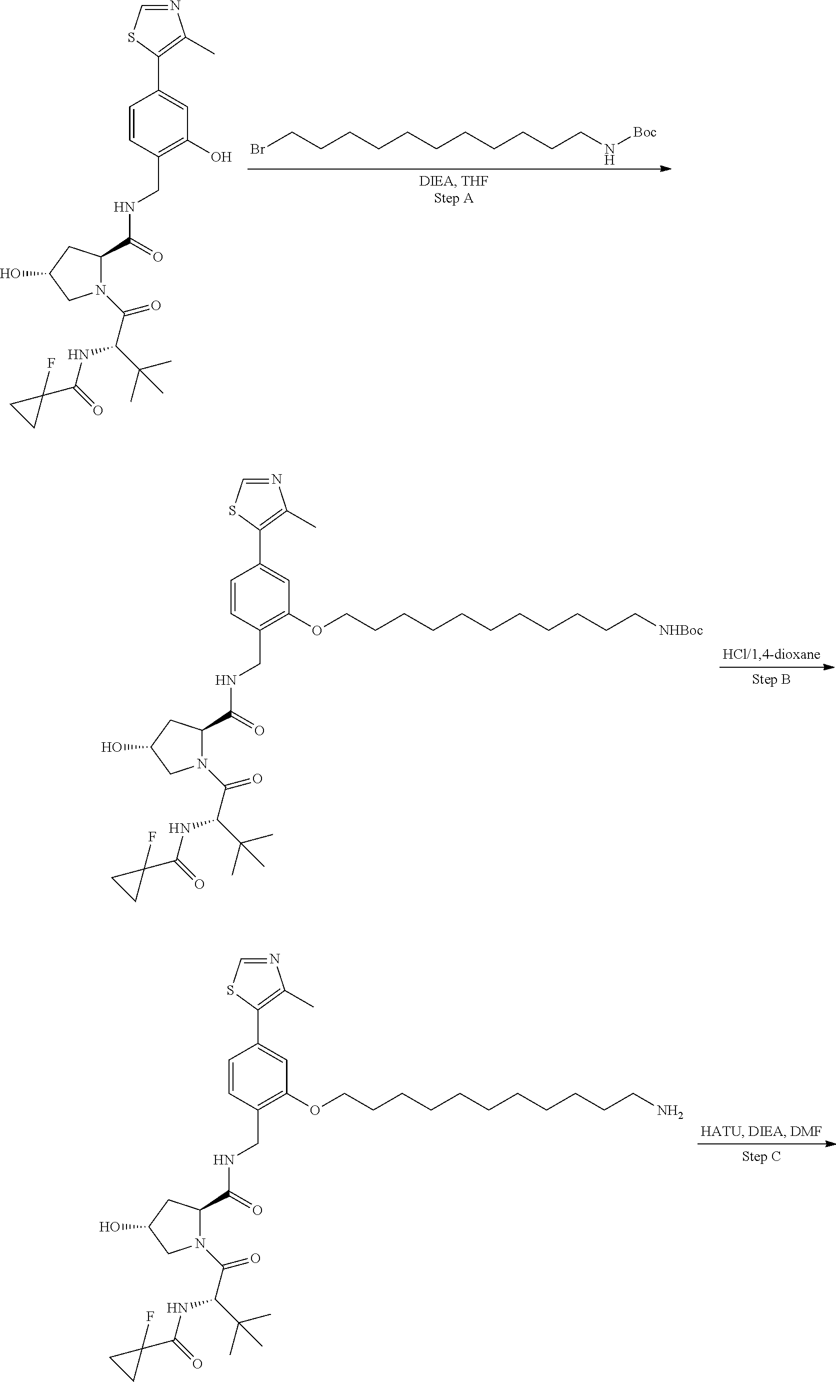 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)NCCCCCCCCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCN)c1]

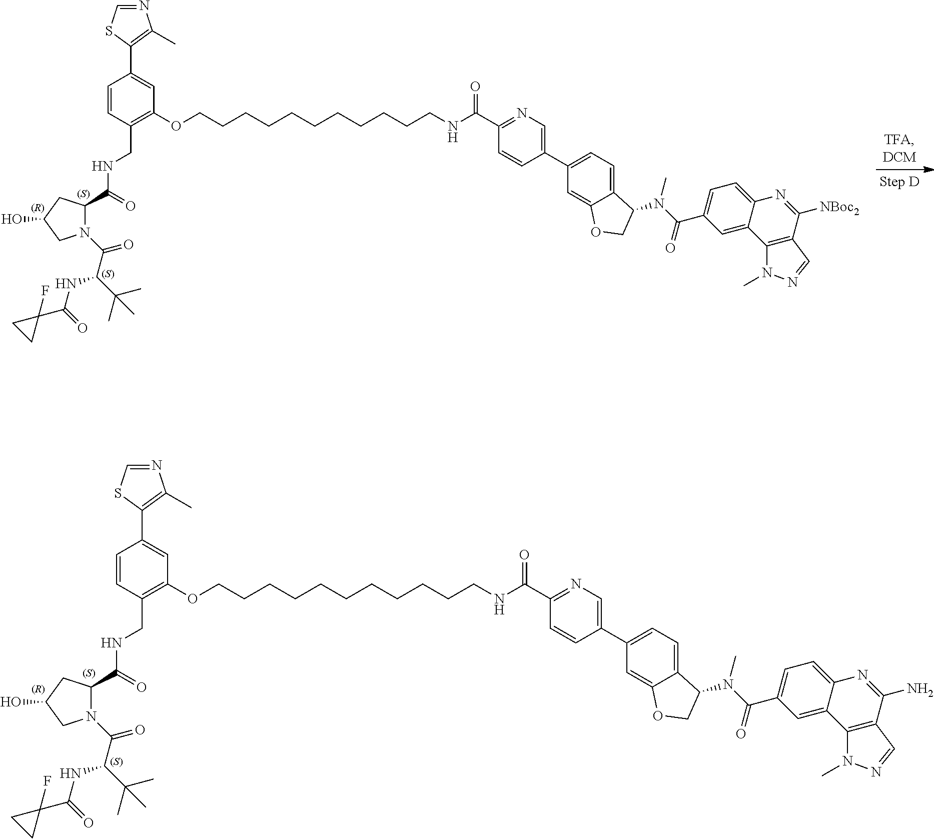

Example A5 was synthesized following the same procedures as described in Example A1 (5.0 mg, 48% yield over 4 steps) as a white solid. LC-MS (ESI): mass calcd, for C₆₄H₇₆FN₁₁O₈S, 1177.5; m/z found, 1178.6 [M+H]⁺.

### Example A6. 4-amino-N—((S)-6-(6-((2-(2-(2-(2-(((28,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N,1-dimethyl-1H-pyrazolo[4,3-c]quinoline-8-carboxamide

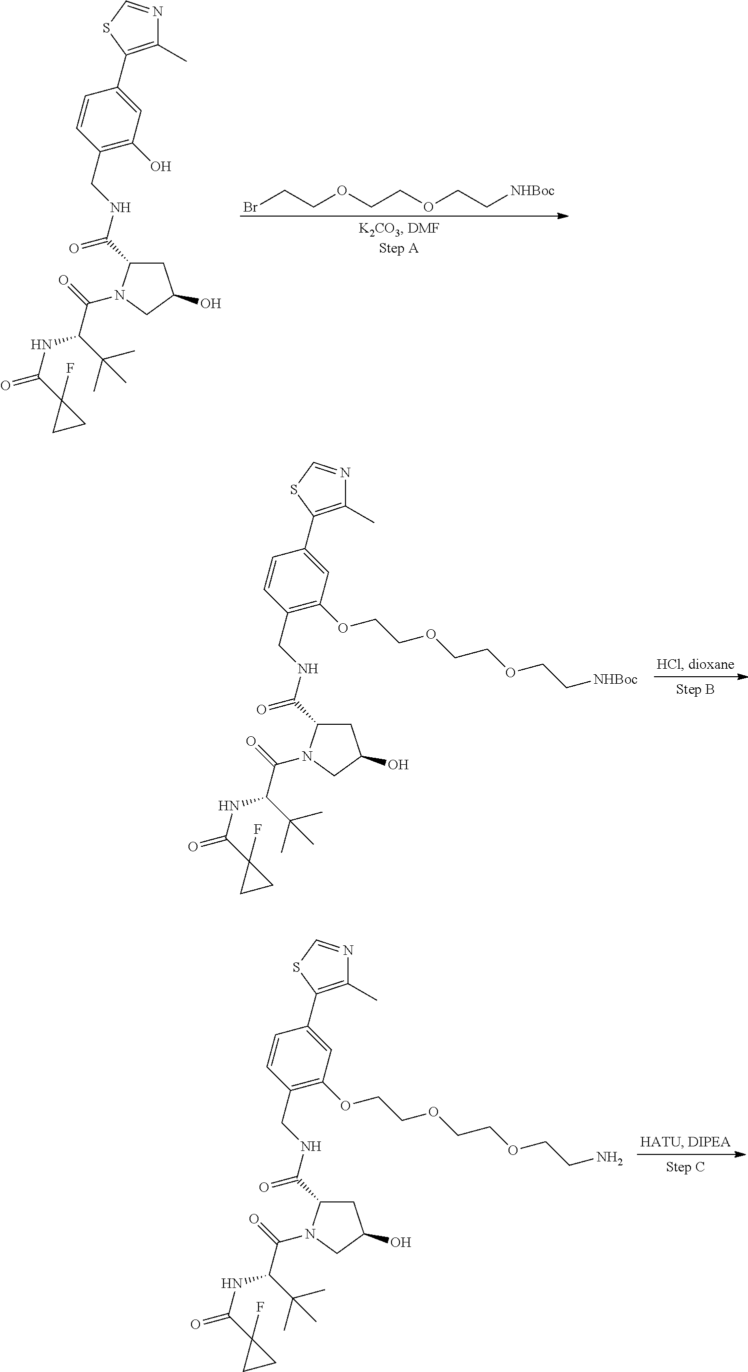 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)NCCOCCOCCBr ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCN)c1]

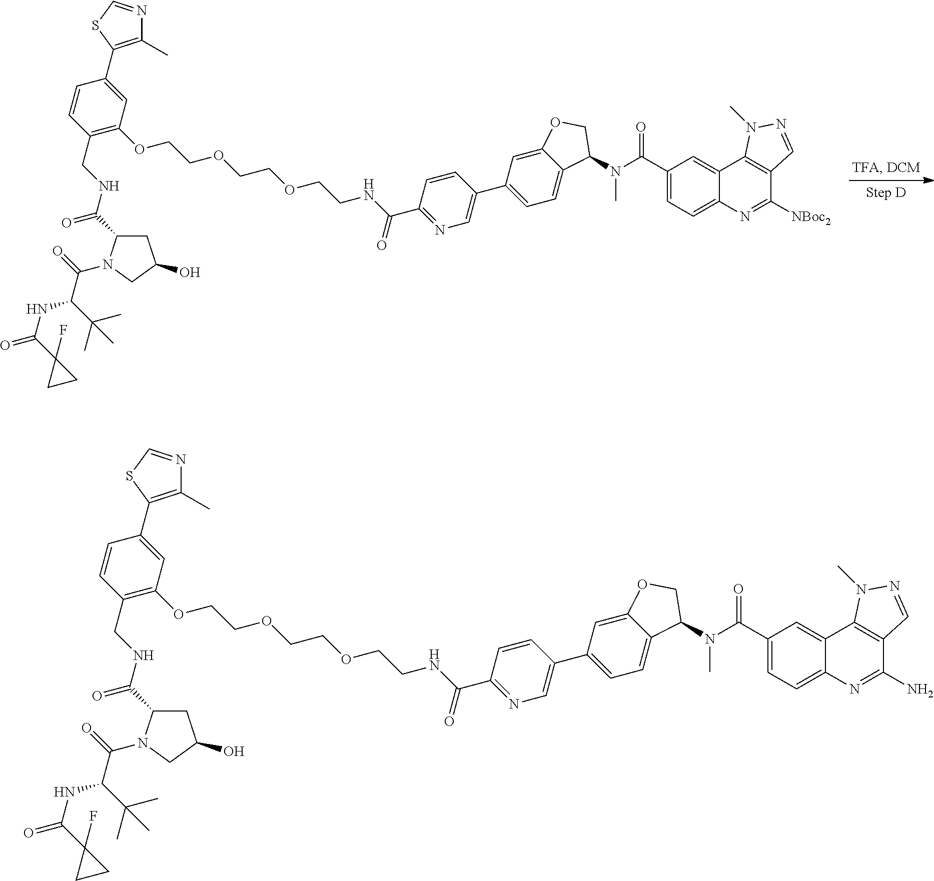

Example A6 was synthesized following the same procedures as described in Example A1 (2.6 mg, 4% yield over 4 steps) as a white solid. LC-MS (ESI): mass calcd, for C₅₉H₆₆FN₁₁O₁₀S, 1139.47; m/z found, 1040.8 [M+H]⁺.

### Example A7. 4-amino-N—((S)-6-(6-((2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl) yl)pyridin-3-yl)-2,3-dihydrobenzofuran-3-yl)-N,1-dimethyl-1H-pyrazolo[4,3-c]quinoline-8-carboxamide

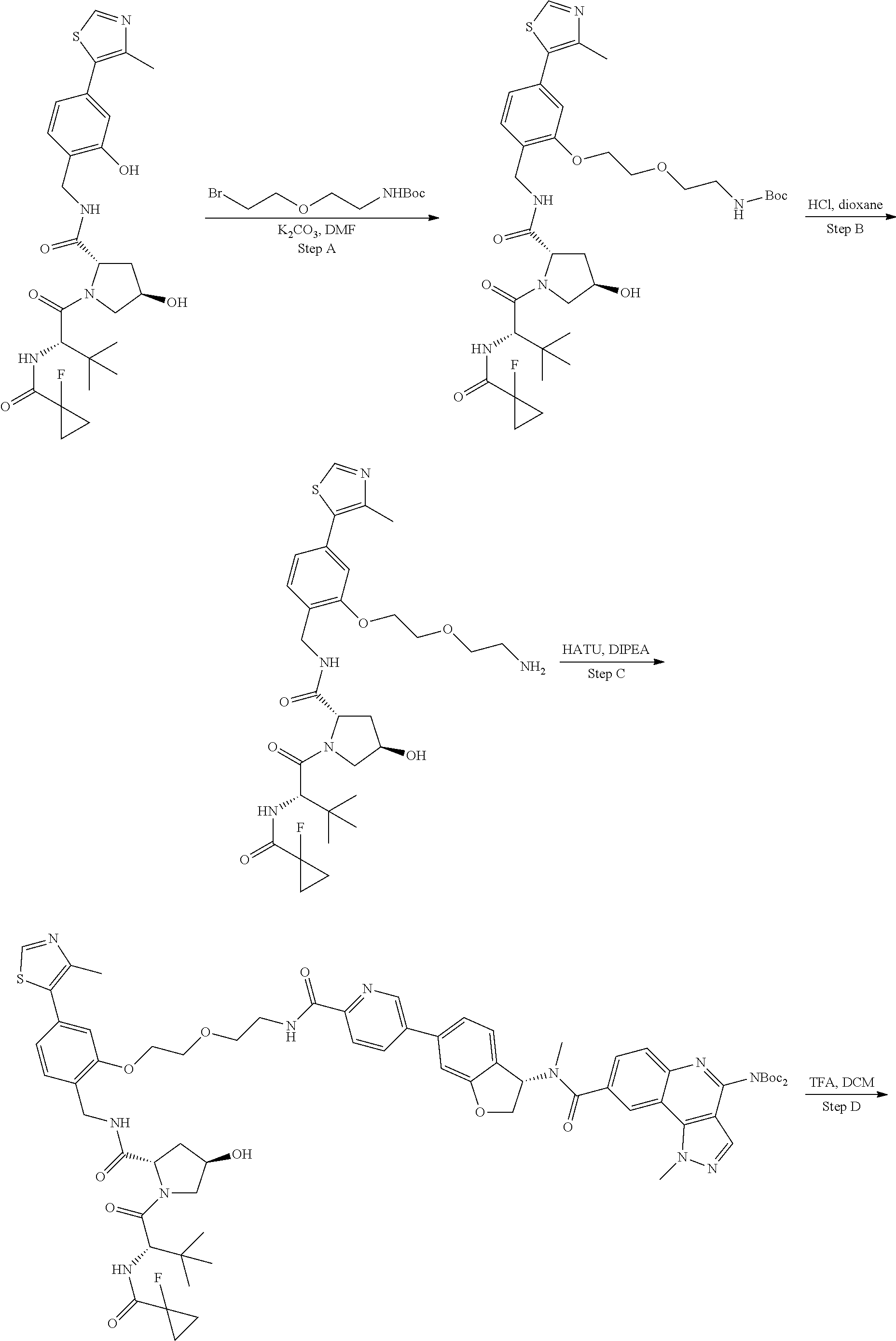

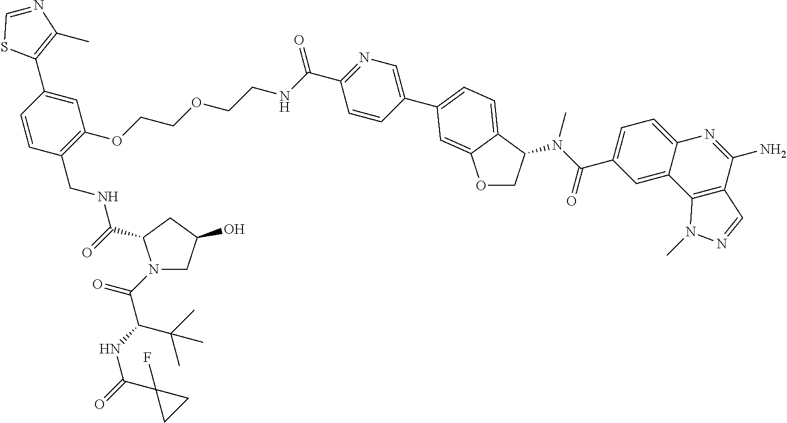 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCNC(=O)c2ccc(-c3ccc4c(c3)OC[C@H]4N(C)C(=O)c3ccc4nc(N)c5cnn(C)c5c4c3)cn2)c1]

Example A7 was synthesized following the same procedures as described in Example A1 (2.0 mg, 7.3% yield over 4 steps) as a white solid. LC-MS (ESI): mass calcd. For C₅₇H₆₂FN₁₁O₉S, 1095.4; m/z found, 1096.3 [M+H]⁺.

### Example A8. 4-amino-N—((S)-6-(6-((2-(2-(((2S,4R)-1-((S)-2-(1-fluorocyclopropane-1-carboxamido)-3,3-dimethylbutanoyl)-4-hydroxypyrroli-dine-2-carboxamido)methyl)-5-(4-methylthiazol-5-yl)phenoxy)ethyl)carbamoyl)-pyridin-3-yl)-2,3-dihydrobenzofuran-3-yl)-N,1-dimethyl-1H-pyrazolo[4,3-c]quinoline-8-carboxamide

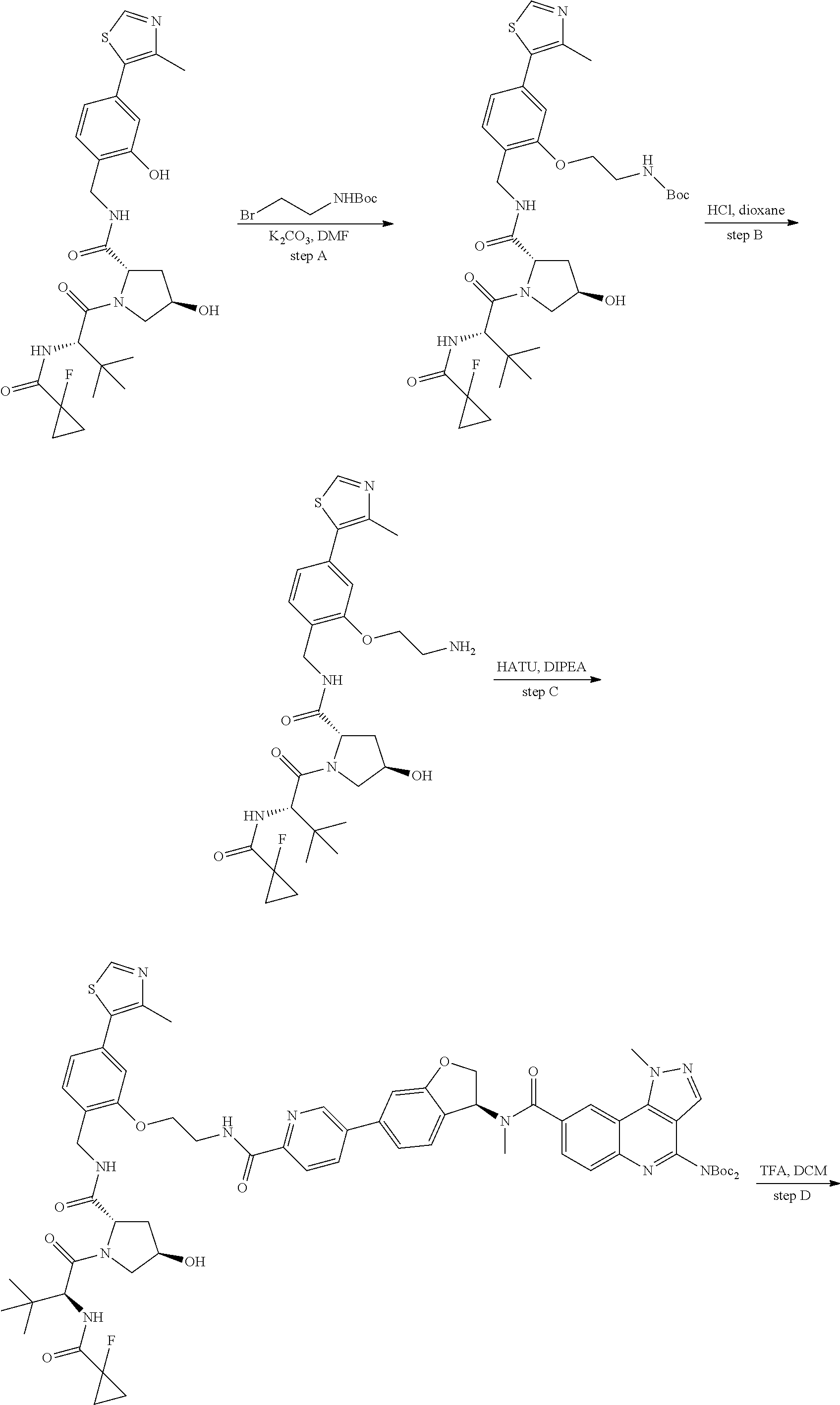

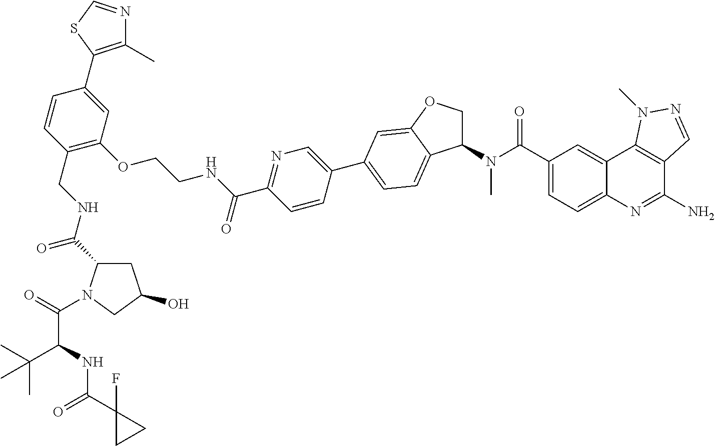 [SMILES: Cc1ncsc1-c1ccc(CNC(=O)[C@@H]2C[C@@H](O)CN2C(=O)[C@@H](NC(=O)C2(F)CC2)C(C)(C)C)c(OCCNC(=O)c2ccc(-c3ccc4c(c3)OC[C@H]4N(C)C(=O)c3ccc4nc(N)c5cnn(C)c5c4c3)cn2)c1]

Example A8 was synthesized following the same procedures as described in Example A1 (3 mg, 10% yield over 4 steps) as a white solid. LC-MS (ESI): mass calcd, for C₅₅H₅₈FN₁₁O₈S, 1051.4; m/z found, 1052 [M+H]⁺.

### Example A9. 4-amino-N—((S)-6-(6-((17-(2-(((2S,4R)-1-((S)-2-(1-fluorocyclopropane-1-carboxamido)-3,3-dimethylbutanoyl)-4-hyroxypyrrolidine-2-carboxamido)methyl)-5-(4-methylthiazol-5-yl)phenoxy)-3,6,9,12,15-pentaoxah-eptadecyl)carbamoyl)pyridin-3-yl)-2,3-dihydrobenzofuran-3-yl)-N-methyl-1,3-dihydrofuro[3,4-c][1,7]naphthyridine-8-carboxamide

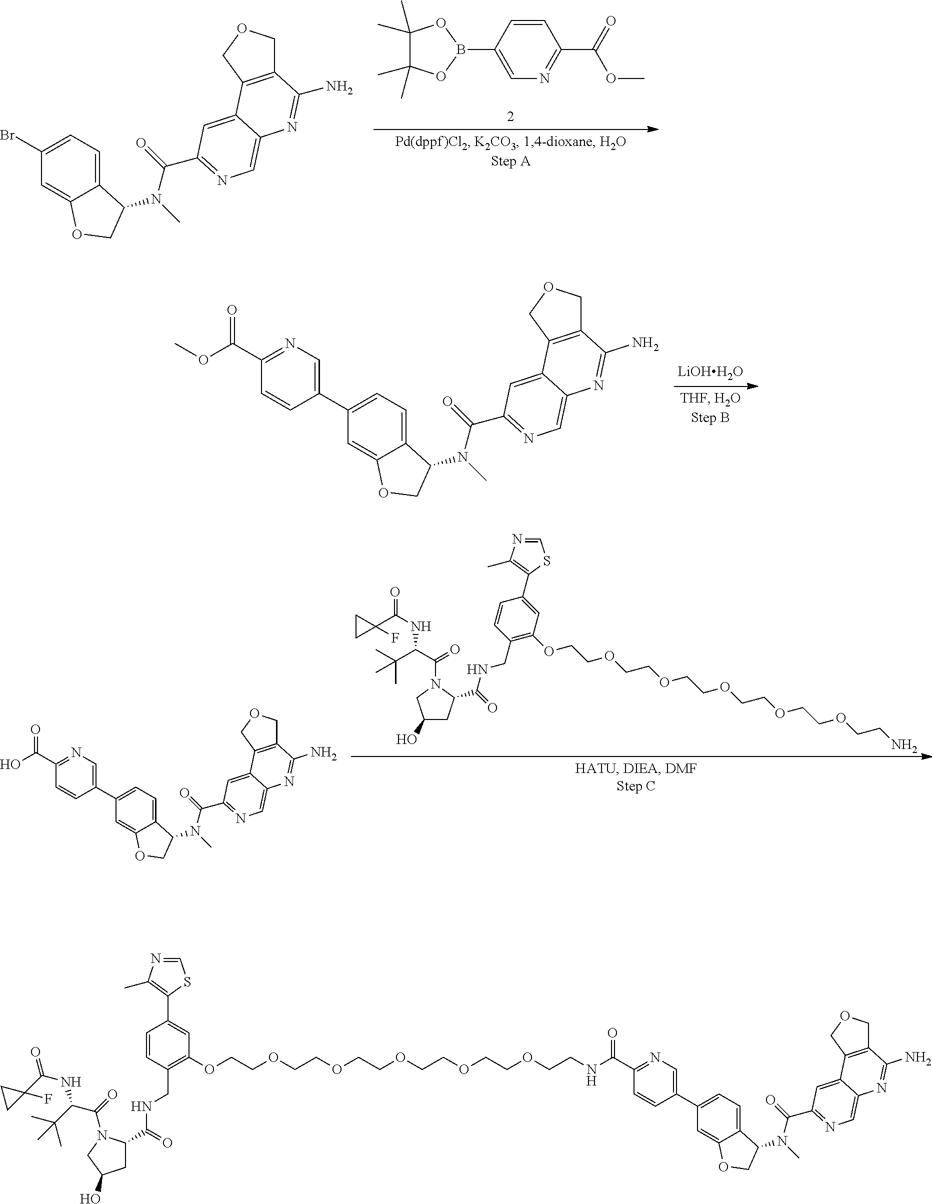

Step A: methyl(S)-5-(3-(4-amino-N-methyl-1,3-dihydrofuro[3,4-c][1,7]naphthyridine-8-carboxamido)-2,3-dihydrobenzofuran-6-yl)picolinate

To a mixture of(S)-4-amino-N-(6-bromo-2,3-dihydrobenzofuran-3-yl)-N-methyl-1,3-dihydrofuro[3,4-c][1,7]naphthyridine-8-carboxamide (120 mg, 0.27 mmol, 1.0 eq.), methyl 5-(4,4,5,5-tetramethyl-1,3,2-dioxaborolan-2-yl)picolinate (86 mg, 0.32 mmol, 1.2 eq.), K₂CO₃ (150 mg, 1.1 mmol, 4.0 eq.) and Pd(dppf)Cl₂ (20.1 mg, 27 μmol, 0.1 eq.) in 1,4-dioxane (5.0 mL) and Water (1.0 mL) was stirred at 90° C. under N₂ for 3 hours. The resulting solution was diluted with 30 mL of water and extracted with EtOAc (30 mL×3). The combined organic layers were washed with brine (20 mL), dried over Na₂SO₄ and concentrated under reduced pressure. The residue was purified by flash column chromatography on silica gel (100-200 mesh silica gel, 0-25% EtOAc in PE) to give methyl(S)-5-(3-(4-amino-N-methyl-1,3-dihydrofuro[3,4-c][1,7]naphthyridine-8-carboxamido)-2,3-dihydrobenzofuran-6-yl)picolinate (90 mg, 67% yield) as a yellow solid. LC-MS (ESI): mass calcd, for C₂₇H₂₃N₅O₅, 497.17; m/z found, 498.1 [M+H]⁺.

Step B: (S)-5-(3-(4-amino-N-methyl-1,3-dihydrofuro[3,4-c][1,7]naphthyridine-8-carboxamido)-2,3-dihydrobenzofuran-6-yl)picolinic acid

To a stirred mixture of methyl(S)-5-(3-(4-amino-N-methyl-1,3-dihydrofuro[3,4-c][1,7]naphthyridine-8-carboxamido)-2,3-dihydrobenzofuran-6-yl)picolinate (90 mg, 0.18 mmol, 1.0 eq.) in THF (4 mL)/H₂O (1 mL) was added LiOH (15 mg, 0.36 mmol, 2.0 eq.). The resulting mixture was stirred at room temperature for 15 hours. The mixture was concentrated under reduced pressure to give(S)-5-(3-(4-amino-N-methyl-1,3-dihydrofuro[3,4-c][1,7]naphthyridine-8-carboxamido)-2,3-dihydrobenzofuran-6-yl)picolinic acid (80 mg, 92% yield) as a white solid. LC-MS (ESI): mass calcd, for C₂₆H₂₁N₅O₅, 483.15; m/z found, 483.2 [M+H]⁺.

Step C: 4-amino-N—(S)-6-(6-(17-(2-((25, 4R)-1-(S)-2-(1-fluorocyclopropane-1-carboxamido)-3,3-dimethylbutanoyl)-4-hyroxypyrrolidine-2-carboxamido)methyl)-5-(4-methylthiazol-5-yl)phenoxy)-3,6,9,12, 15-pentaoxah-eptadecyl)carbamoyl)pyridin-3-yl)-2,3-dihydrobenzofuran-3-yl)-N-methyl-1,3-dihydrofuro[3,4-c][1,7]naphthyridine-8-carboxamide

Example A9 was synthesized following the same procedures as described in Example A1 (3 mg, 14% yield) as a white solid. LC-MS (ESI): mass calcd, for C₆₄H₇₇FN₁₀O₁₄S, 1260.53; m/z found, 1260.5 [M+H]⁺.

### Example A10. 4-amino-N—((S)-6-(6-((14-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)-3,6,9,12-tetraoxatetradecyl)carbamoyl)pyridin-3-yl)-2,3-dihydrobenzofuran-3-yl)-N-methyl-1,3-dihydrofuro[3,4-c][1,7]naphthyridine-8-carboxamide

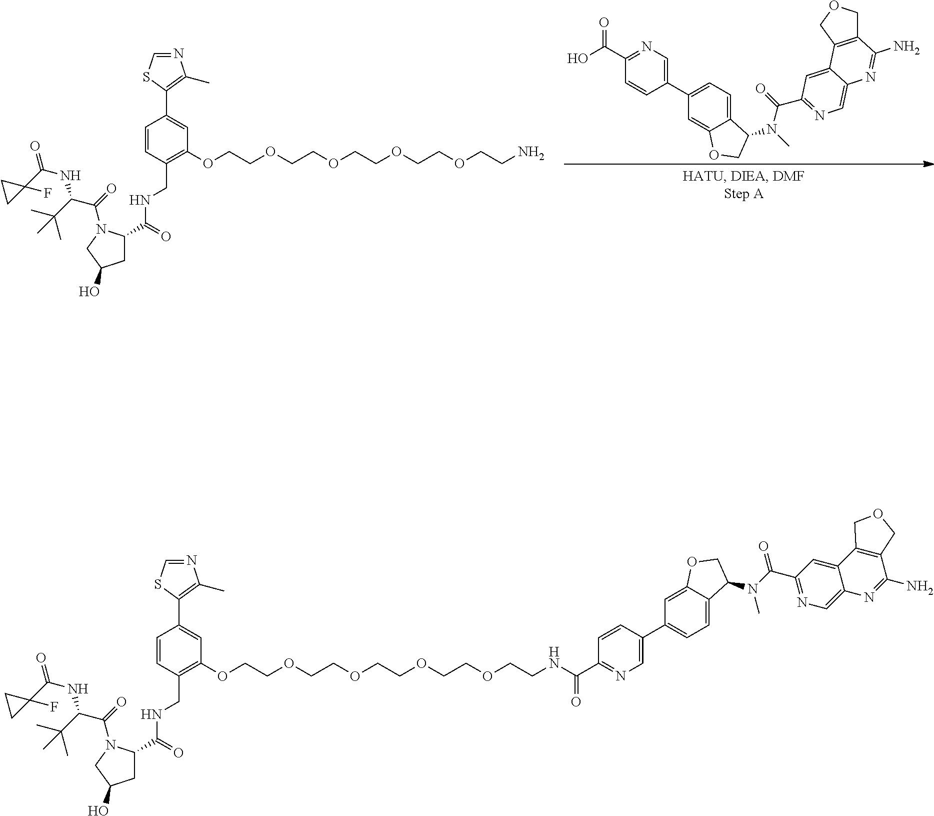 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCN)c1 ; CN(C(=O)c1cc2c3c(c(N)nc2cn1)COC3)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)c2ccc(-c3ccc4c(c3)OC[C@H]4N(C)C(=O)c3cc4c5c(c(N)nc4cn3)COC5)cn2)c1]

Example A10 was synthesized following the same procedures as described in Example A9 (1.2 mg, 8% yield) as a white solid. LC-MS (ESI): mass calcd, for C₆₂H₇₃FN₁₀O₁₃S, 1216.51; m/z found, 1217.4 [M+H]⁺.

### Example A11. 4-amino-7-fluoro-N—((S)-6-(6-((17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl) carbamoyl)pyridin-3-yl)-2,3-dihydrobenzofuran-3-yl)-N-methyl-1,3-dihydrofuro[3,4-c]aninoline-8-carboxamide

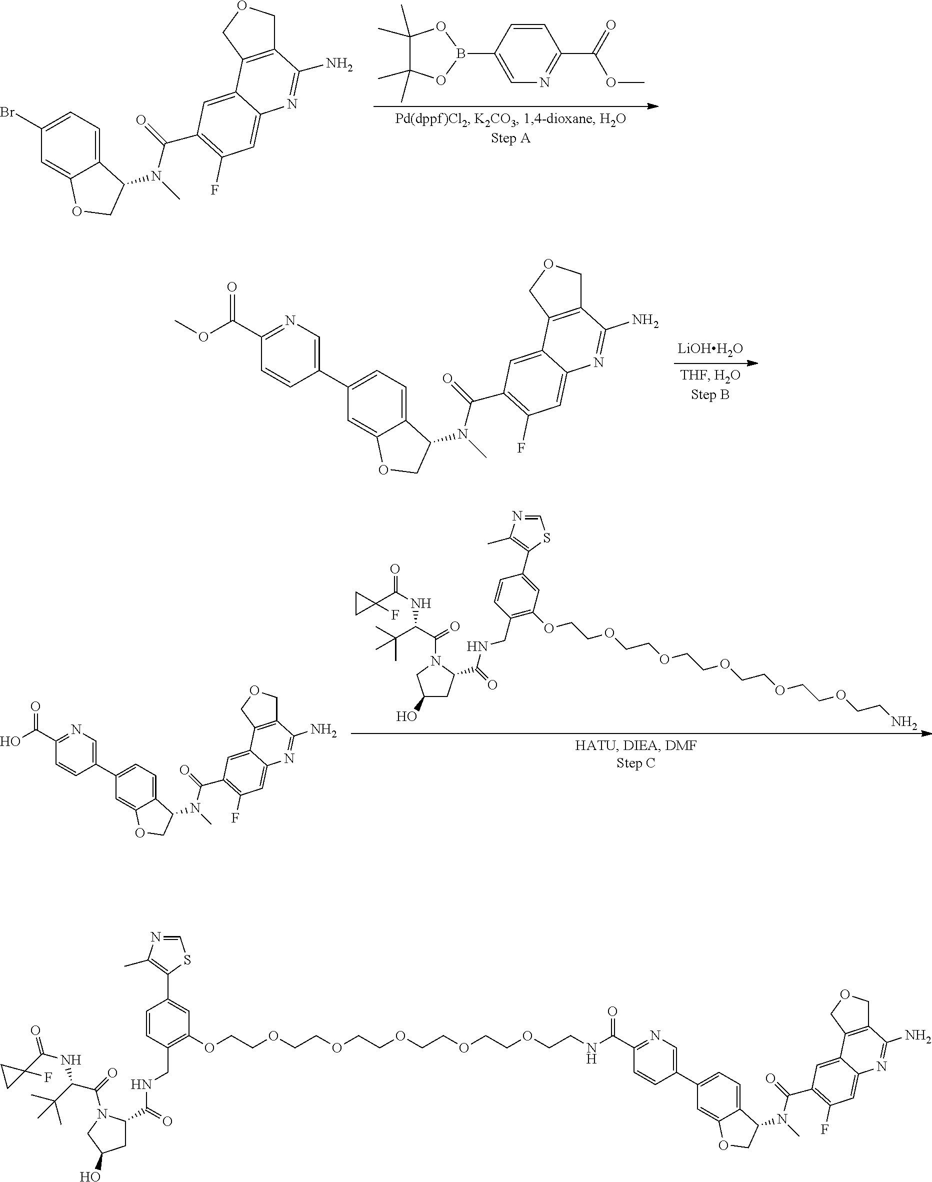

Step A: tert-butyl 3-(3-bromo-2-(hydroxymethyl)phenyl)-3-hydroxypyrrolidine-1-carboxylate

To a mixture of(S)-4-amino-N-(6-bromo-2,3-dihydrobenzofuran-3-yl)-7-fluoro-N-methyl-1,3-dihydrofuro[3,4-c]quinoline-8-carboxamide (160 mg, 349 μmol, 1.0 eq.), methyl 5-(4,4,5,5-tetramethyl-1,3,2-dioxaborolan-2-yl)picolinate (110 mg, 419 μmol, 1.2 eq.) and K₂CO₃ (145 mg, 1.05 mmol, 3.0 eq.) in 1,4-dioxane (2.0 mL) and H₂O (0.1 mL) was added Pd(dppf)Cl₂ (25 mg, 34.9 μmol, 0.1 eq.) under nitrogen atmosphere. Then was stirred at 100° C. for 3 hours. The mixture was poured into water and extracted with EtOAc (20 mL×3). The combined organic layers were washed with brine, dried over anhydrous Na₂SO₄ and concentrated under reduced pressure. The residue was purified by flash chromatographer (eluent, 0~25% EtOAc in PE) to give the methyl(S)-5-(3-(4-amino-7-fluoro-N-methyl-1,3-dihydrofuro[3,4-c]quinoline-8-carboxamido)-2,3-dihydrobenzofuran-6-yl)picolinate (130 mg, 72% yield) as a white solid.

LC-MS (ESI): mass calcd, for C₂₈H₂₃FN₄O₅, 514.17; m/z found, 514.9 [M+H]⁺.

Step B: (S)-5-(3-(4-amino-7-fluoro-N-methyl-1,3-dihydrofuro[3,4-c]quinoline-8-carboxamido)-2,3-dihydrobenzofuran-6-yl)picolinic acid

To a mixture of methyl(S)-5-(3-(4-amino-7-fluoro-N-methyl-1,3-dihydrofuro[3,4-c]quinoline-8-carboxamido)-2,3-dihydrobenzofuran-6-yl)picolinate (100 mg, 194 μmol, 1.0 eq.) in THF (2.0 mL) was added H₂O (0.4 mL) and LiOH·H₂O (7 mg, 292 μmol, 1.5 eq.) at room temperature. Then the mixture was stirred at room temperature for 2 hours. The mixture was concentrated under reduced pressure to dryness to give the(S)-5-(3-(4-amino-7-fluoro-N-methyl-1,3-dihydrofuro[3,4-c]quinoline-8-carboxamido)-2,3-dihydrobenzofuran-6-yl)picolinic acid (85 mg, 87% yield) as a white solid.

LC-MS (ESI): mass calcd, for C₂₇H₂₁FN₄O₅, 500.1; m/z found, 501.2 [M+H]⁺.

Step C: 4-amino-7-fluoro-N—(S)-6-(6-((17-(2-(((2S,4R)-1-(S)-2-(1-fluorocyclopropane-I-carboxamido)-3,3-dimethylbutanoyl)-4-hydroxypyrrolidi-ne-2-carboxamido)methyl)-5-(4-methylthiazol-5-yl)phenoxy)-3,6,9, 12,15-pentaoxaheptadecyl)carbamoyl)pyridin-3-yl)-2,3-dihydrobenzofuran-3-yl)-N-methyl-1,3-dihydrofuro[3,4-c]quinoline-8-carboxamide

To a mixture of (2S,4R)—N-(2-((17-amino-3,6,9,12,15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20.0 mg, 25.1 μmol, 1.0 eq.), (S)-5-(3-(4-amino-7-fluoro-N-methyl-1,3-dihydrofuro[3,4-c]quinoline-8-carboxamido)-2,3-dihydrobenzofuran-6-yl)picolinic acid (12.6 mg, 25.1 μmol, 1.0 eq.) and DIEA (9.7 mg, 13.1 μL, 75.4 μmol, 3.0 eq.) in DMF (1.0 mL) was added HATU (14.3 mg, 37.7 μmol, 1.5 eq) at room temperature. Then the mixture was stirred at room temperature for 1 hour. The mixture was filtered and purified by prep-HPLC(C18, 10~95%, MeCN in H₂O with 0.1% FA) to give the 4-amino-7-fluoro-N—((S)-6-(6-((17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamoyl)pyridin-3-yl)-2,3-dihydrobenzofuran-3-yl)-N-methyl-1,3-dihydrofuro[3,4-c]quinoline-8-carboxamide (6 mg, 18% yield) as a white solid.

LC-MS (ESI): mass calcd, for C₆₅H₇₇F₂N₉O₁₄S, 1277.5; m/z found, 1278.7 [M+H]⁺.

### Example A12. 4-amino-7-fluoro-N—((S)-6-(6-((14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12-tetraoxatetradecyl) carbamoyl)pyridin-3-yl)-2,3-dihydrobenzofuran-3-yl)-N-methyl-1,3-dihydrofuro[3,4-c]quinoline-8-carboxamide

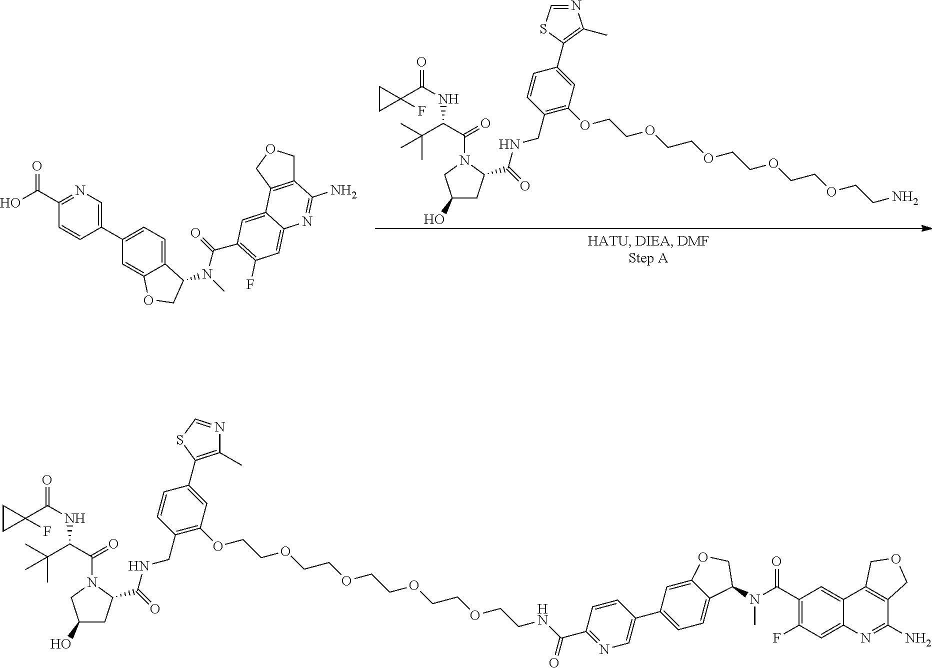 [SMILES: CN(C(=O)c1cc2c3c(c(N)nc2cc1F)COC3)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCNC(=O)c2ccc(-c3ccc4c(c3)OC[C@H]4N(C)C(=O)c3cc4c5c(c(N)nc4cc3F)COC5)cn2)c1]

Example A12 was synthesized following the same procedures as described in Example A11 (10 mg, 15% yield) as a white solid. LC-MS (ESI): mass calcd, for C₆₃H₇₃F₂N₉O₁₃S, 1233.5; m/z found, 1234.2 [M+H]⁺.

### Example A13. 2-amino-N—((S)-6-(6-((14-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)-3,6,9,12-tetraoxatetradecyl)carbamoyl)pyridin-3-yl)-2,3-dihydrobenzofuran-3-yl)-N,3-dimethylqui-noline-6-carboxamide

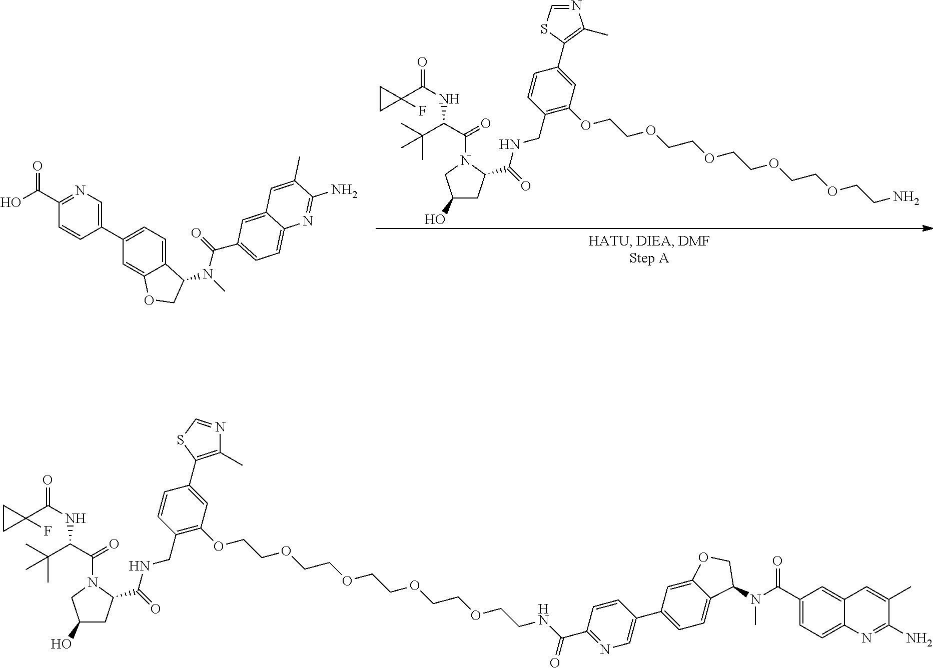 [SMILES: Cc1cc2cc(C(=O)N(C)[C@@H]3COc4cc(-c5ccc(C(=O)O)nc5)ccc43)ccc2nc1N ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCN)c1 ; Cc1cc2cc(C(=O)N(C)[C@@H]3COc4cc(-c5ccc(C(=O)NCCOCCOCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N]

Step A: 2-amino-N—((S)-6-(6-((14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12-tetraoxatetradecyl)carbamoyl)pyridin-3-yl)-2,3-dihydrobenzofuran-3-yl)-N,3-dimethylquinoline-6-carboxamide

To a solution of (2S,4R)—N-(2-((14-amino-3,6,9,12-tetraoxatetradecyl)oxy)-4-(4-methylthiazol-5-yl)benzyl)-1-((S)-2-(1-fluorocyclopropane-1-carboxamido)-3,3-dimethylbutanoyl)-4-hydroxy-pyrrolidine-2-carboxamide (20 mg, 26.6 μmol, 1.0 eq.), (S)-5-(3-(2-amino-N,3-dimethylquinoline-6-carboxamido)-2,3-dihydrobenzofuran-6-yl)picolinic acid (12 mg, 26.6 μmol, 1.0 eq.), HATU (15 mg, 39.9 μmol, 1.5 eq.) in DMF (1 mL) was added DIEA (10.3 mg, 79.8 μmol, 3.0 eq.) at 0° C. The reaction mixture was stirred for 1 hour under N₂ atmosphere. The mixture was purified by prep-HPLC(C18, 20~95%, MeCN in H₂O with 0.1% HCOOH) to give 2-amino-N—((S)-6-(6-((14-(2-(((2S,4R)-1-((S)-2-(1-fluorocyclopropane-1-carboxamido)-3,3-dimethyl-butanoyl)-4-hydroxypyrrolidine-2-carboxamido)methyl)-5-(4-methylthiazol-5-yl)phenoxy)-3,6,9,12-tetraoxatetradecyl)carbamoyl)pyridin-3-yl)-2,3-dihydrobenzofuran-3-yl)-N,3-dimethylquinoline-6-carboxamide (9.5 mg, 30% yield) as a white solid.

LC-MS (ESI): mass calcd, for C₆₄H₇₈FN₉O₁₃S, 1187; m/z found, 1187.9 [M+H]⁺.

### Example A14. 2-amino-N—((S)-6-(6-((17-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)-3,6,9,12,15-pentao-xaheptadecyl)carbamoyl)pyridin-3-yl)-2,3-dihydrobenzofuran-3-yl)-N,3-dimeth-ylquinoline-6-carboxamide

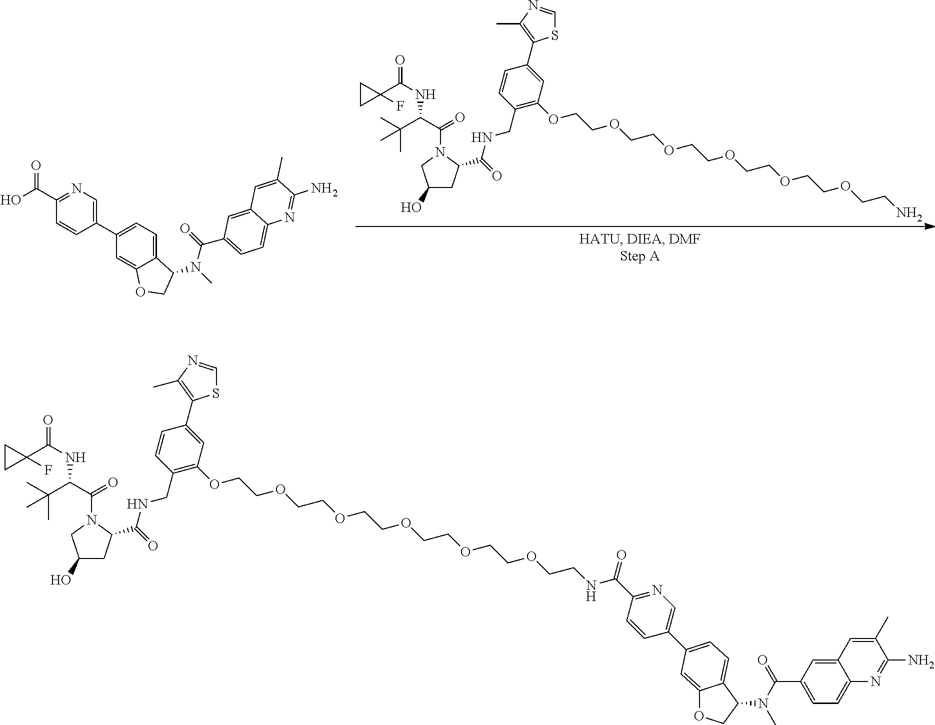 [SMILES: Cc1cc2cc(C(=O)N(C)[C@@H]3COc4cc(-c5ccc(C(=O)O)nc5)ccc43)ccc2nc1N ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOCCOCCN)c1 ; Cc1cc2cc(C(=O)N(C)[C@@H]3COc4cc(-c5ccc(C(=O)NCCOCCOCCOCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N]

Step A: 2-amino-N—(S)-6-(6-(17-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12, 15-pentaoxaheptadecyl)carbamoyl)-pyridin-3-yl)-2,3-dihydrobenzofuran-3-yl)-N,3-dimethylquinoline-6-carboxamide

To a solution of (28,4R)—N-(2-((17-amino-3,6,9,12,15-pentaoxaheptadecyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20 mg, 25.1 μmol, 1.0 eq.), (S)-5-(3-(2-amino-N,3-dimethylquinoline-6-carboxamido)-2,3-dihydrobenzofuran-6-yl)picolinic acid (11 mg, 25.1 μmol, 1.0 eq.), HATU (14 mg, 37.7 μmol, 1.5 eq.) in DMF (1 mL) was added DIEA (9.7 mg, 75.4 μmol, 3.0 eq.) at 0° C. The reaction mixture was stirred for 1 hour under N₂ atmosphere. The mixture was purified by prep-HPLC(C18, 20~95%, MeCN in H₂O with 0.1% HCOOH) to give 2-amino-N—((S)-6-(6-((17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)carbamoyl)pyridin-3-yl)-2,3-dihydrobenzofuran-3-yl)-N,3-dimethylquinoline-6-carboxamide (5.9 mg, 19% yield) as a white solid.

LC-MS (ESI): mass calcd, for C₆₄H₇₈FN₉O₁₃S, 1231.5; m/z found, 1231.8 [M+H]⁺.

### Example A15. 4-amino-N—((S)-6-(6-((9-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy) nonyl)carbamoyl)pyridin-3-yl)-2,3-dihydrobenzofuran-3-yl)-N,1-dimethyl-1H-pyrazolo[4,3-c]quinoline-8-carboxamide

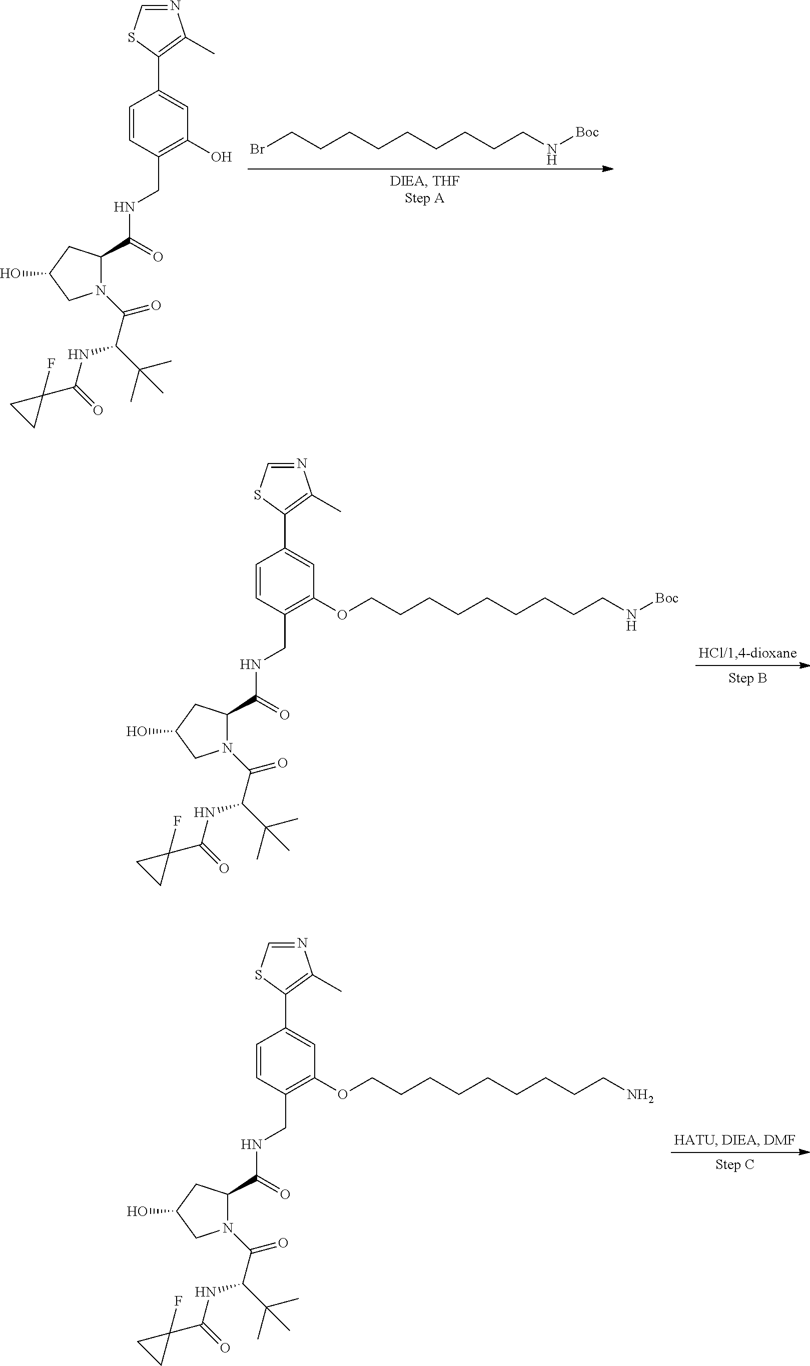 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)NCCCCCCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCN)c1]

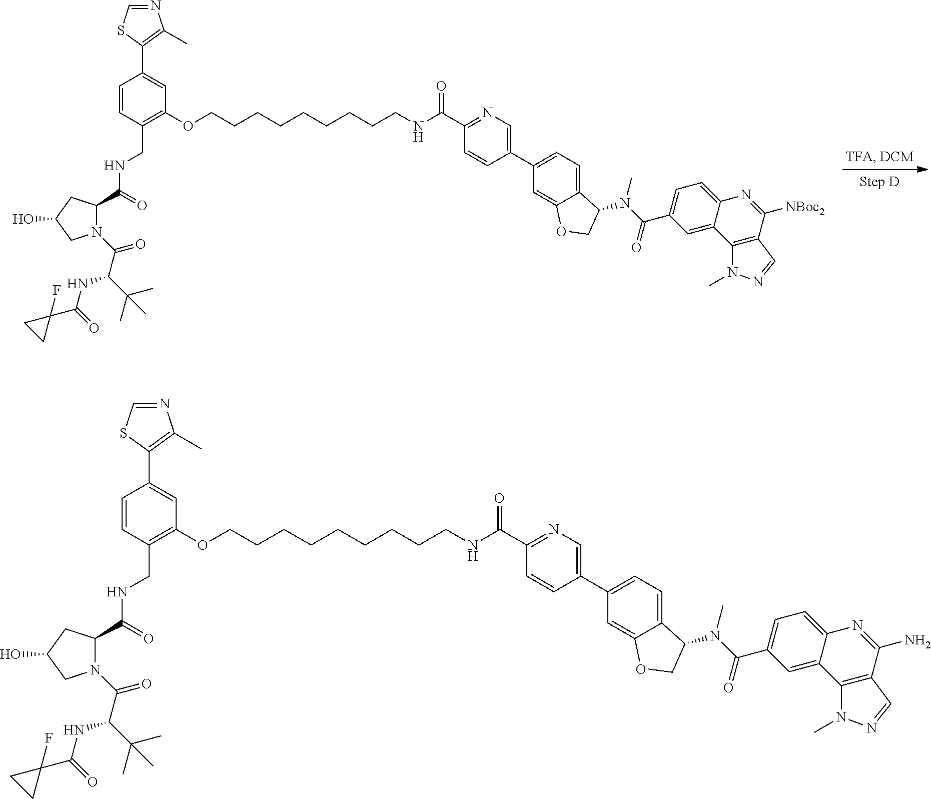

Example A15 was synthesized following the same procedures as described in Example A1 (3 mg, 6% yield over 4 steps) as a white solid. LC-MS (ESI): mass calcd, for C₆₂H₂₂FN₁₁O₈S, 1149.5; m/z found, 1150.7 [M+H]⁺.

### Example A16. 4-amino-N—((S)-6-(6-((10-(2-(((2S,4R)-1-((S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy) decyl)carbamoyl)pyridin-3-yl)-2,3-dihydrobenzofuran-3-yl)-N,1-dimethyl-1H-pyrazolo[4,3-c]quinoline-8-carboxamide

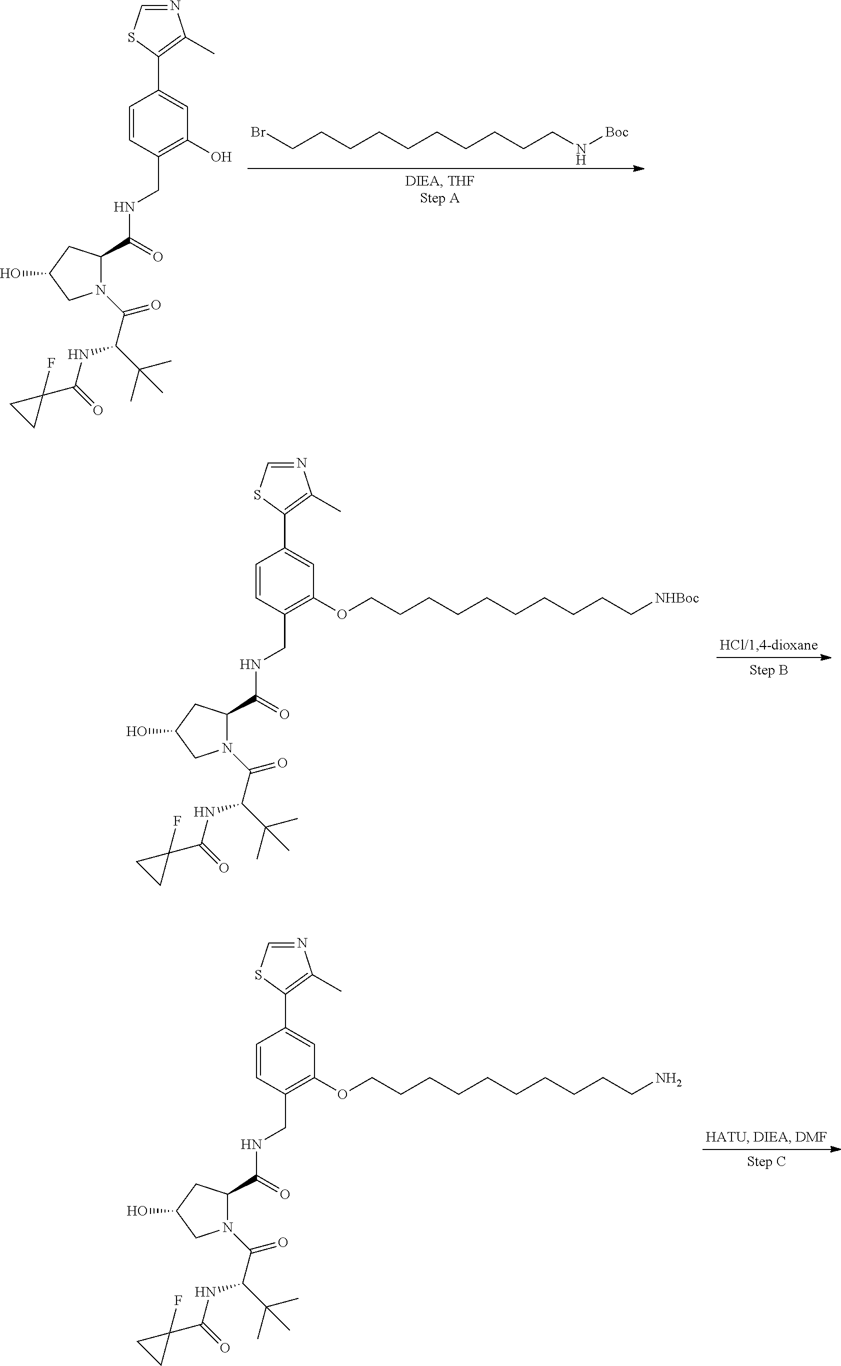 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)NCCCCCCCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCN)c1]

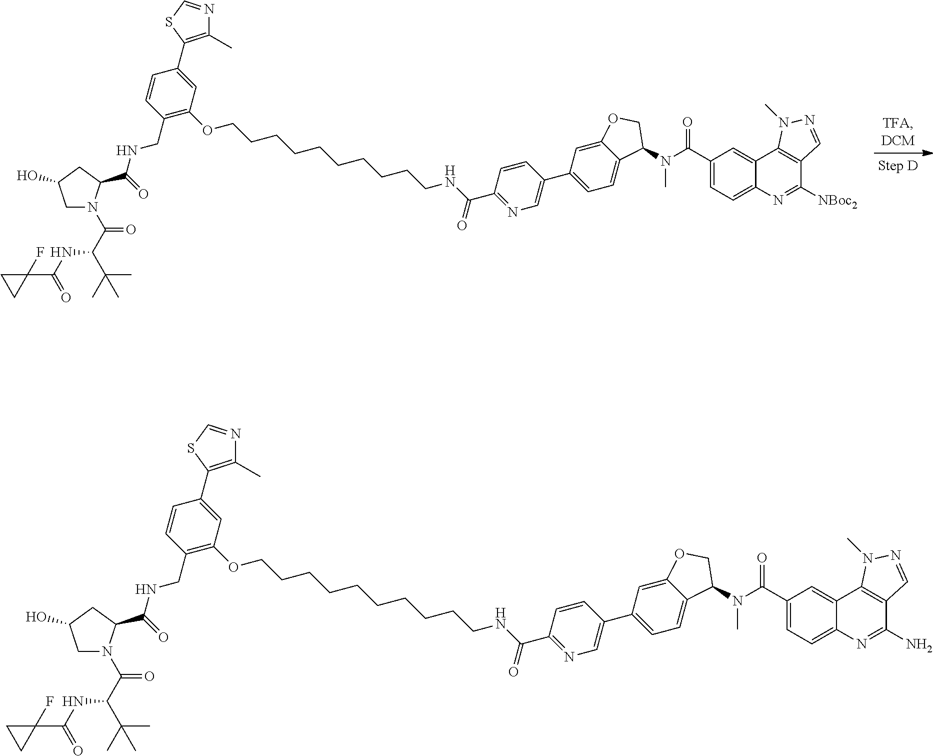

Example A16 was synthesized following the same procedures as described in Example A1 (3 mg, 5% yield over 4 steps) as a white solid. LC-MS (ESI): mass calcd, for C₆₃H₇₄FN₁₁O₈S, 1163.5; m/z found, 1164.6 [M+H]⁺.

### Example A17. 4-amino-N—((S)-6-(6-(4-(2-(2-(2-(2-(2-(((28,4R)-1-((S)-2-(1-fluorocyclopropane-1-carboxamido)-3,3-dimethylbutanoyl)-4-hydroxypyrroli-dine-2-carboxamido)methyl)-5-(4-methylthiazol-5-yl)phenoxy)ethoxy)ethoxy)-ethoxy)ethyl) piperazine-1-carbonyl)pyridin-3-yl)-2,3-dihydrobenzofuran-3-yl)-N,1-dimethyl-1H-pyrazolo[4,3-c]quinoline-8-carboxamide

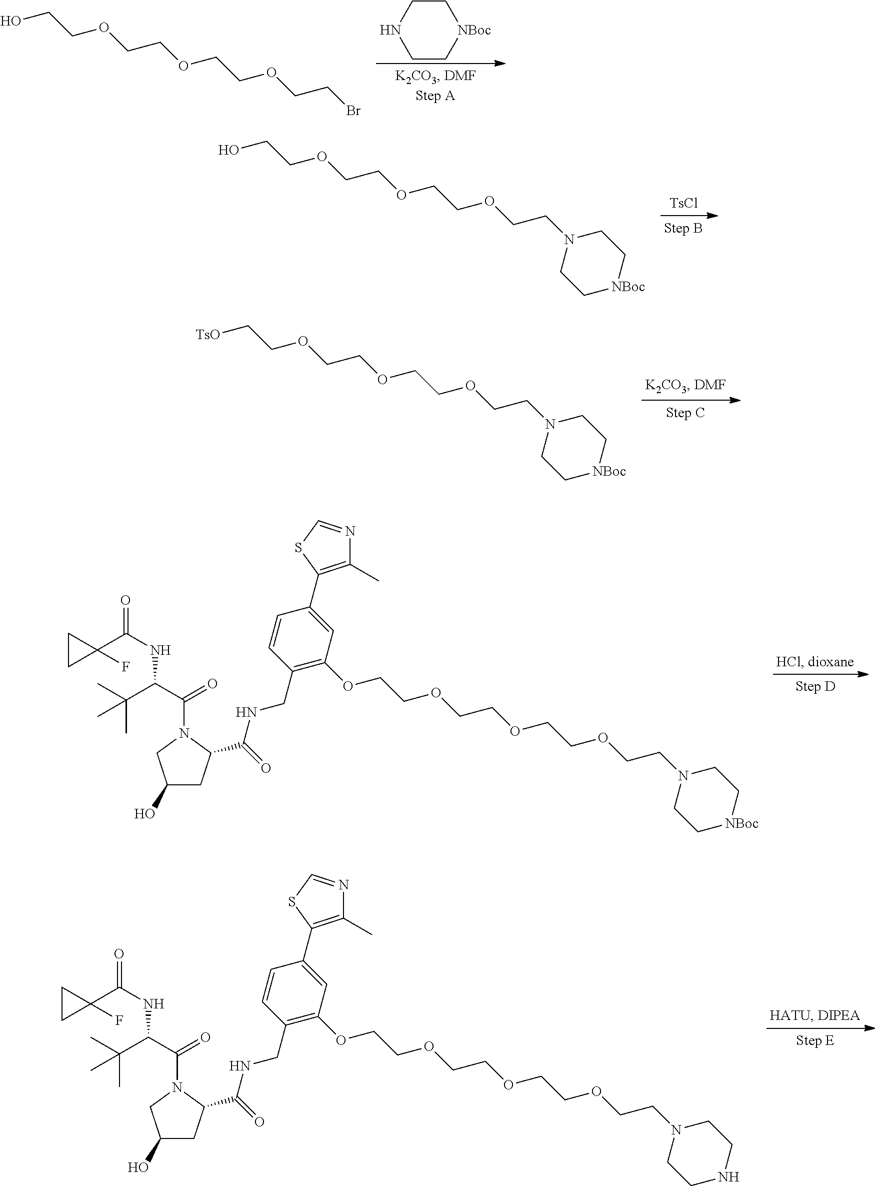 [SMILES: OCCOCCOCCOCCBr ; CC(C)(C)OC(=O)N1CCNCC1 ; CC(C)(C)OC(=O)N1CCN(CCOCCOCCOCCO)CC1 ; Cc1ccc(S(=O)(=O)OCCOCCOCCOCCN2CCN(C(=O)OC(C)(C)C)CC2)cc1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCN2CCN(C(=O)OC(C)(C)C)CC2)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCN2CCNCC2)c1]

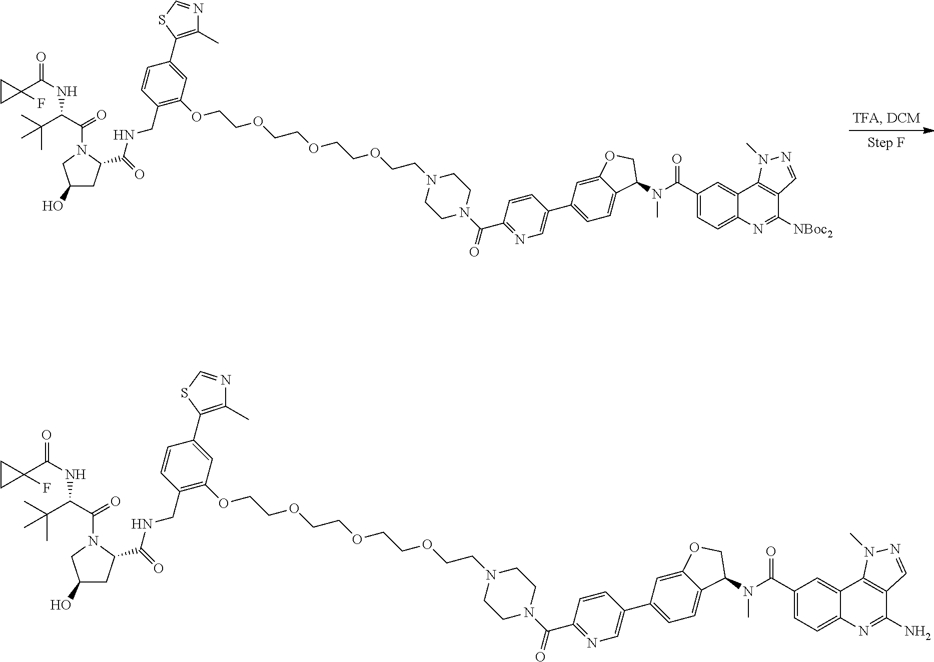

Step A: tert-butyl 4-(2-(2-(2-(2-hydroxyethoxy)ethoxy)ethoxy)ethyl) piperazine-1-carboxylate To a solution of 2-(2-(2-(2-bromoethoxy)ethoxy)ethoxy) ethan-1-ol (100 mg, 0.4 mmol, 1.0 eq.) and tert-Butyl 1-piperazinecarboxylate (80 mg, 0.4 mmol, 1.1 eq.) in MeCN (5 mL) was added K₂CO₃ (162 mg, 1.2 mol, 3.0 eq.). Then the mixture was stirred at 80° C. for 16 hours. The mixture was diluted with water and extracted three times with EtOAc (20 mL×3). The organic phase was dried over Na₂SO₄, filtered and concentrated in vacuum to afford crude tert-butyl 4-(2-(2-(2-(2-hydroxyethoxy)ethoxy)ethoxy)ethyl) piperazine-1-carboxylate (200 mg, 70% purity) as a colorless oil.

LC-MS (ESI): mass calcd, for C₁₇H₃₄N₂O₆, 362.2; m/z found, 363 [M+H]⁺.

Step B: tert-butyl 4-(2-(2-(2-(2-(tosyloxy)ethoxy)ethoxy)ethoxy)ethyl) piperazine-1-carboxylate

To a solution of tert-butyl 4-(2-(2-(2-(2-hydroxyethoxy)ethoxy)ethoxy)ethyl) piperazine-1-carboxylate (100 mg, 70% purity, 1.0 eq.) and TEA (80 μL, 0.6 mmol, 3.0 eq.) in MeCN (10 mL) was added TsCl (55 mg, 0.29 mmol, 1.5 eq.),followed by DMAP (47 mg, 0.39 mmol, 2.0 eq.). Then the mixture was stirred at room temperature for 3 hours. The mixture was diluted with water and extracted three times with EtOAc. The organic phase was dried over Na₂SO₄, filtered and concentrated in vacuum. The residue was purified on silica gel eluting (0~4% MeOH in DCM) to afford tert-butyl 4-(2-(2-(2-(2-(tosyloxy)ethoxy)ethoxy)ethoxy)ethyl) piperazine-1-carboxylate (75 mg, 75% yield) as a white solid.

LC-MS (ESI): mass calcd, for C₂₄H₄₀N₂O₈S, 516.3; m/z found, 517 [M+H]⁺.

Step C: tert-butyl 4-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl) piperazine-1-carboxylate

To a solution of tert-butyl 4-(2-(2-(2-(2-(tosyloxy)ethoxy)ethoxy)ethoxy)ethyl) piperazine-1-carboxylate (75 mg, 0.2 mmol, 2.0 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl) pyrrolid-ine-2-carboxamide (40 mg, 0.08 mmol, 1.0 eq.) in DMF (2 mL) was added K₂CO₃ (41 mg, 0.3 mol, 4.0 eq.). Then the mixture was stirred at 50° C. for 6 hours. The mixture was diluted with water and extracted three times with EtOAc (20 mL×3). The organic phase was dried over Na₂SO₄, filtered and concentrated in vacuum. The residue was purified on silica gel eluting (0~9% MeOH in DCM) to afford tert-butyl 4-(2-(2-(2-(2-(2-(((2S,4R)-1-((S)-2-(1-fluorocyclopropane-1-carboxamido)-3,3-dimethyl-butanoyl)-4-hydroxypyrrolidine-2-carboxamido)methyl)-5-(4-methylthiazol-5-yl)phenoxy)ethoxy)ethoxy)ethoxy)ethyl) piperazine-1-carboxylate (50 mg, 76% yield) as a colorless oil.

LC-MS (ESI): mass calcd, for C₄₃H₆₅FN₆O₁₀S, 876.5; m/z found, 877 [M+H]⁺.

### Steps D-F

### Example A17 was synthesized following the same procedures as described in Example A1 (3 mg, 4% yield over 3 steps) as a white solid. LC-MS (ESI): mass calcd, for C₆₅H₇₇FN₁₂O₁₁S, 1252.5; m/z found, 1253.6 [M+H]⁺

### Example A18. 4-amino-N—((S)-6-(6-(9-(2-(2-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)-3,9-diazaspiro[5.5]undecane-3-carbonyl)pyridin-3-yl)-2,3-dihydrobenzofuran-3-yl)-N,1-dimethyl-1H-pyrazolo[4,3-c]quinoline-8-carboxamide

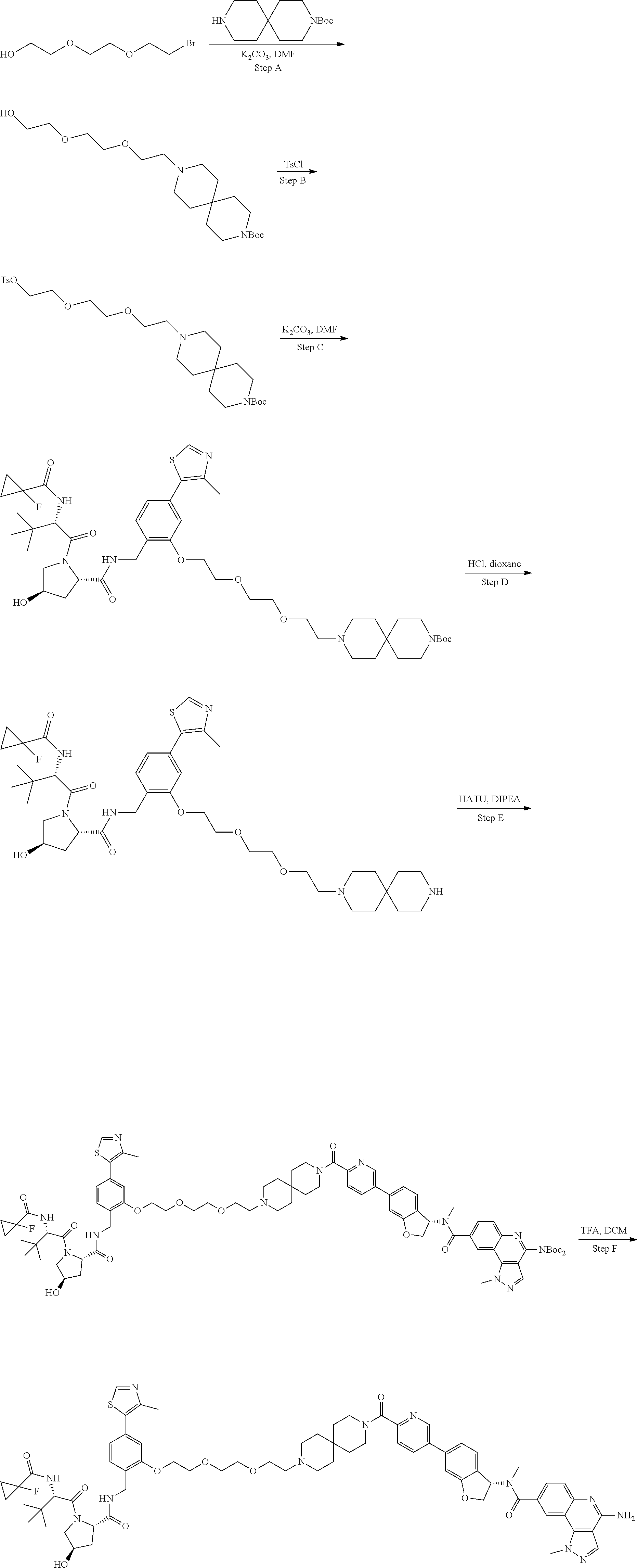

Example A18 was synthesized following the same procedures as described in Example A17 (3 mg, 4% yield over 6 steps) as a white solid. LC-MS (ESI): mass calcd, for C₆₈H₈₁FN₁₂O₁₀S, 1276.6; m/z found, 1277.4 [M+H]⁺.

### Example A19. 4-amino-N—((S)-6-(6-((13-(2-(((2R,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)tridecyl)-carbamoyl)pyridin-3-yl)-2,3-dihydrobenzofuran-3-yl)-N,1-dimethyl-III-pyrazolo[4,3-c]quinoline-8-carboxamide

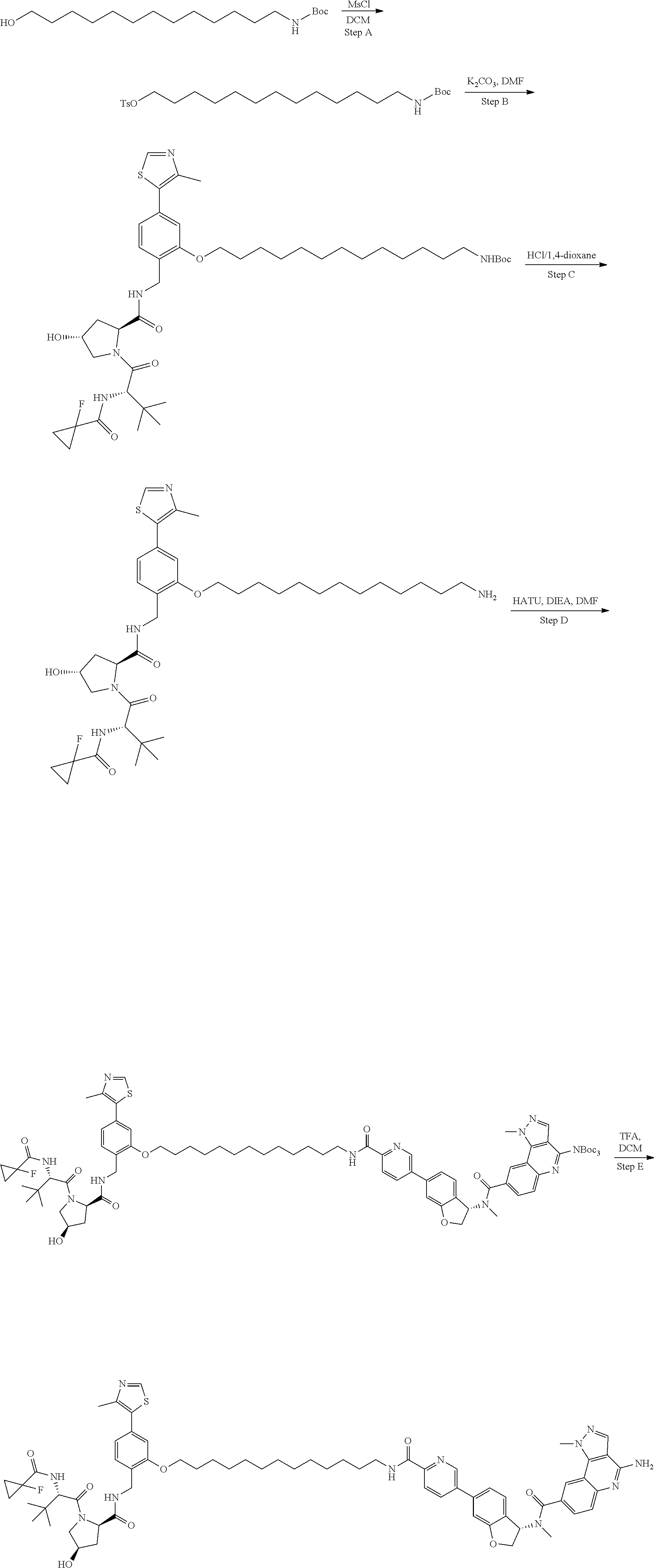

Step A: 13-((tert-butoxycarbonyl)amino) tridecyl 4-methylbenzenesulfonate

To a solution of tert-butyl (13-hydroxytridecyl)carbamate (100 mg, 0.3 mmol, 1.0 eq.) in DMF (20 mL) was added DIEA (130 mg, 1 mmol, 3.0 eq.) and TsCl (90 mg, 0.5 mmol, 1.6 eq.) at 0° C. The reaction mixture was stirred at 0° C. for 3 hours under N₂ atmosphere. The mixture was diluted with water and extracted with EtOAc (20 mL×3). The combined organic layers were washed with brine, dried over anhydrous Na₂SO₄ and concentrated under reduced pressure. The residue was purified by flash chromatographer (eluent, 0~50% EtOAc in PE) to give (13-((tert-butoxycarbonyl)amino) tridecyl 4-methylbenzenesulfonate (90 mg, 60% yield) as a solid.

LC-MS (ESI): mass calcd, for C25H43NO₅S, 469.3; m/z found, 470.3 [M+H]⁺.

Step B: tert-butyl (13-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)tridecyl)carbamate

To a solution of 13-((tert-butoxycarbonyl)amino) tridecyl 4-methylbenzenesulfonate (30 mg, 0.06 mmol, 1.0 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (30 mg, 0.06 mmol, 1.0 eq.) in DMF (5 mL) was added K₂CO₃ (25 mg, 0.18 mmol, 3.0 eq.). The mixture was stirred at 60° C. for 16 hours under N₂ atmosphere. The mixture was diluted with water (30 mL) and extracted with DCM (20 mL×3). The combined organic layers were washed with brine, dried over anhydrous Na₂SO₄ and concentrated under reduced pressure. The residue was purified by flash chromatographer (eluent, 0~10% MeOH in DCM) to give tert-butyl (13-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)tridecyl)carbamate (20 mg, 40% yield) as a white solid.

LC-MS (ESI): mass calcd, for C₄₄H₆₈FN₅O₇S, 830.5; m/z found, 831.5 [M+H]⁺.

### Steps C-E

Example A19 was synthesized following the same procedures as described in Example A1 (4.0 mg, 11% yield over 3 steps) as a white solid. LC-MS (ESI): mass calcd. for C₆₆H₈₀FN₁₁O₈S, 1205.99; m/z found, 1207.0 [M+H]⁺.

### Example A20. 4-amino-N—((S)-6-(6-((14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) tetradecyl)carbamoyl)pyridin-3-yl)-2,3-dihydrobenzofuran-3-yl)-N-methyl-1,3-dihydrofuro[3,4-c][1,7]naphthyridine-8-carboxamide

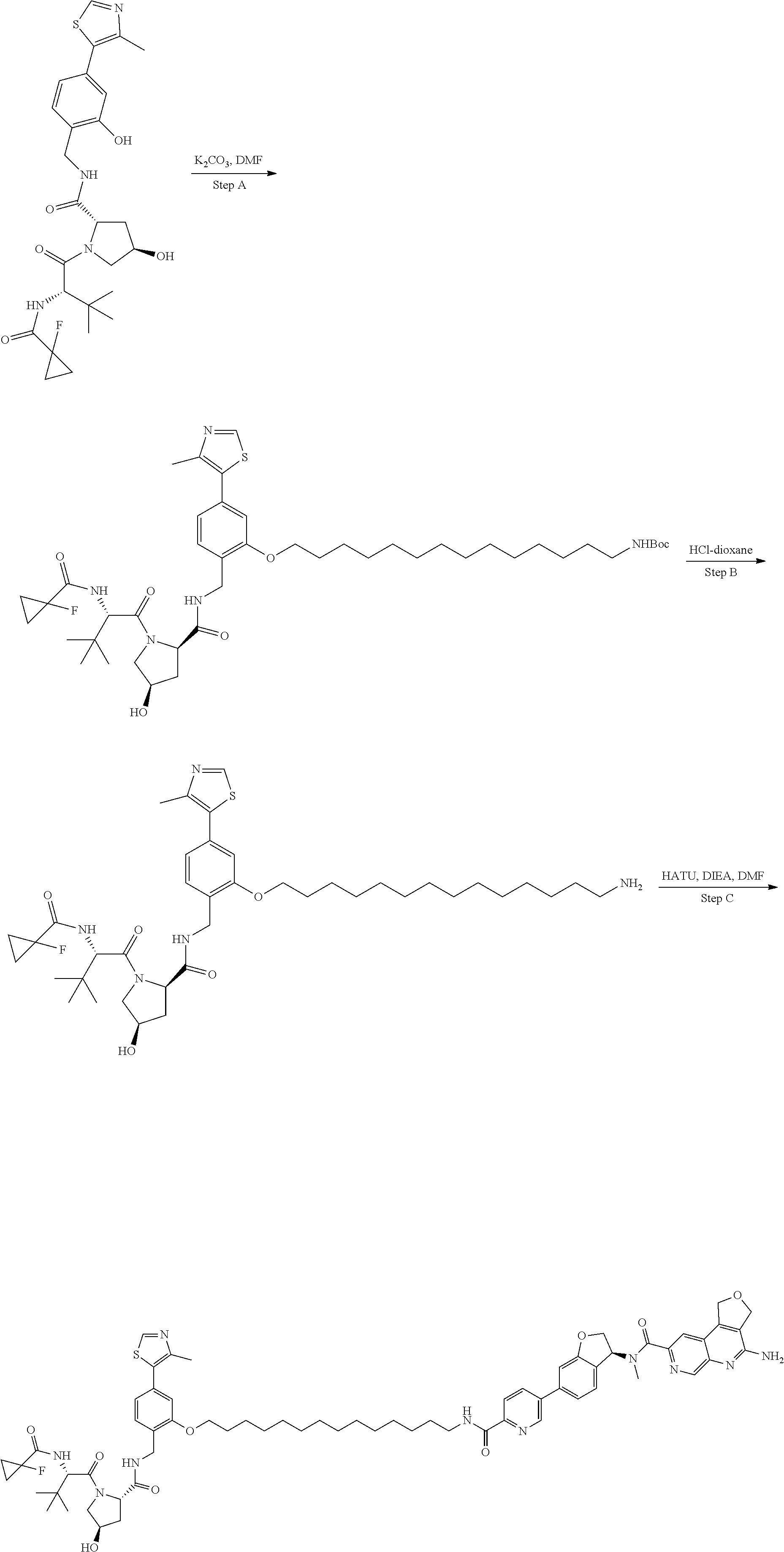 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@H]2C[C@@H](O)CN2C(=O)[C@@H](NC(=O)C2(F)CC2)C(C)(C)C)c(OCCCCCCCCCCCCCCNC(=O)OC(C)(C)C)c1 ; Cc1ncsc1-c1ccc(CNC(=O)[C@H]2C[C@@H](O)CN2C(=O)[C@@H](NC(=O)C2(F)CC2)C(C)(C)C)c(OCCCCCCCCCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCCNC(=O)c2ccc(-c3ccc4c(c3)OC[C@H]4N(C)C(=O)c3cc4c5c(c(N)nc4cn3)COC5)cn2)c1]

Example A20 was synthesized following the same procedures as described in Example A1 (4.0 mg, 11% yield over 3 steps) as a white solid. LC-MS (ESI): mass calcd. for C₆₆H₈₁FN₁₀O₉S, 1208.59; m/z found, 1209.7 [M+H]⁺.

### Example A21. 4-amino-7-fluoro-N—((S)-6-(6-((14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) tetradecyl)carbamoyl)pyridin-3-yl)-2,3-dihydrobenzofuran-3-yl)-N-methyl-1,3-dihydrofuro[3,4-c]quinoline-8-carboxamide

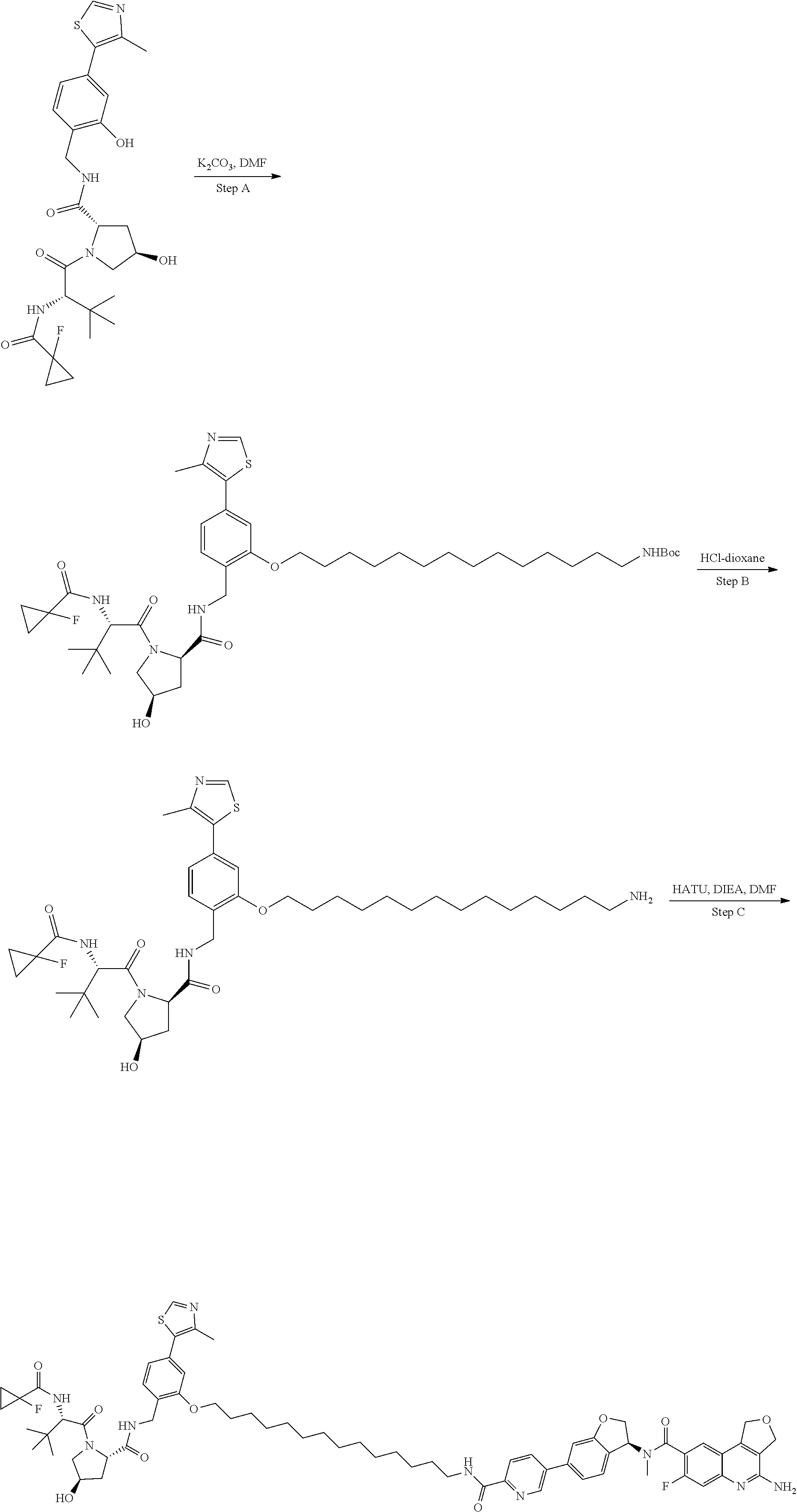 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@H]2C[C@@H](O)CN2C(=O)[C@@H](NC(=O)C2(F)CC2)C(C)(C)C)c(OCCCCCCCCCCCCCCNC(=O)OC(C)(C)C)c1 ; Cc1ncsc1-c1ccc(CNC(=O)[C@H]2C[C@@H](O)CN2C(=O)[C@@H](NC(=O)C2(F)CC2)C(C)(C)C)c(OCCCCCCCCCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCCCCCCCNC(=O)c2ccc(-c3ccc4c(c3)OC[C@H]4N(C)C(=O)c3cc4c5c(c(N)nc4cc3F)COC5)cn2)c1]

Example A21 was synthesized following the same procedures as described in Example A11 (6.1 mg, 4% yield over 3 steps) as a white solid. LC-MS (ESI): mass calcd. for C₆₇H₈₁F₂N₉O₉S, 1225.58; m/z found, 1226.8 [M+H]⁺.

### Example A22. 2-amino-N—((S)-6-(6-((14-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) tetradecyl)carbamoyl)pyridin-3-yl)-2,3-dihydrobenzofuran-3-yl)-N,3-dimethylquinoline-6-carboxamide

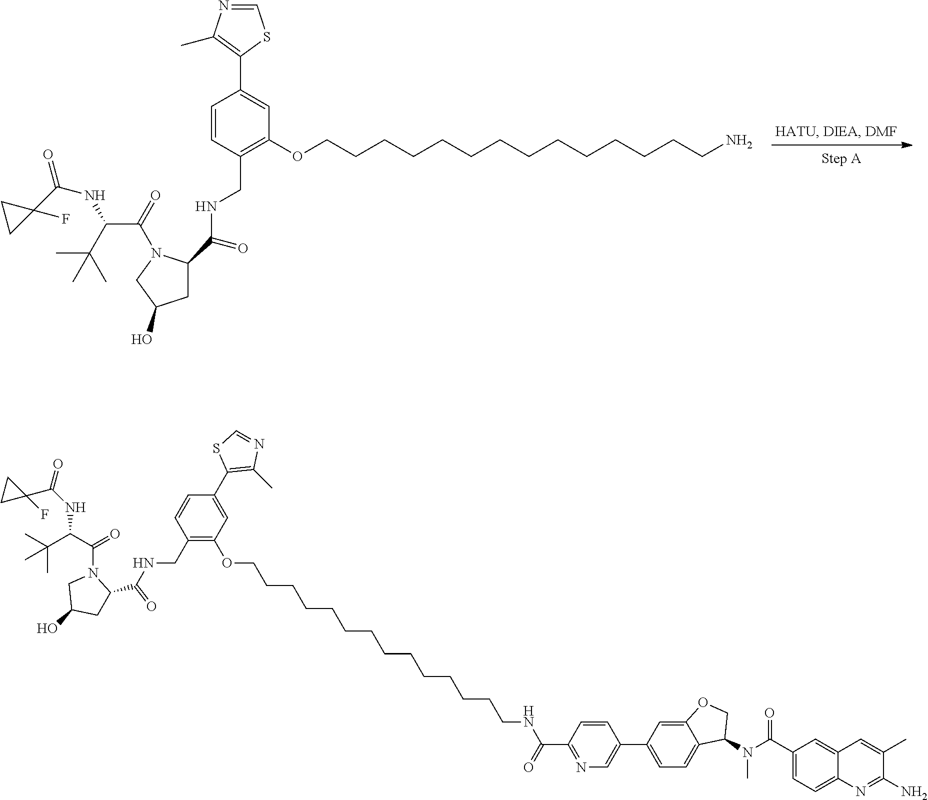 [SMILES: Cc1ncsc1-c1ccc(CNC(=O)[C@H]2C[C@@H](O)CN2C(=O)[C@@H](NC(=O)C2(F)CC2)C(C)(C)C)c(OCCCCCCCCCCCCCCN)c1 ; Cc1cc2cc(C(=O)N(C)[C@@H]3COc4cc(-c5ccc(C(=O)NCCCCCCCCCCCCCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N]

Example A22 was synthesized following the same procedures as described in Example A21 (6 mg, 11% yield) as a white solid. LC-MS (ESI): mass calcd, for C₆₆H₈₂FN₉O₈S, 1179.60; m/z found, 1180.5 [M+H]⁺.

### Example A23. 4-amino-N—((S)-6-(6-((3-(2-(2-(((2S,4R)-1-((S)-2-(1-fluorocyclopropane-1-carboxamido)-3,3-dimethylbutanoyl)-4-hydroxypyrroli-dine-2-carboxamido)methyl)-5-(4-methylthiazol-5-yl)phenoxy)ethoxy) propyl)-carbamoyl)pyridin-3-yl)-2,3-dihydrobenzo-furan-3-yl)-N,1-dimethyl-1H-pyrazolo[4,3-c]quinoline-8-carboxamide

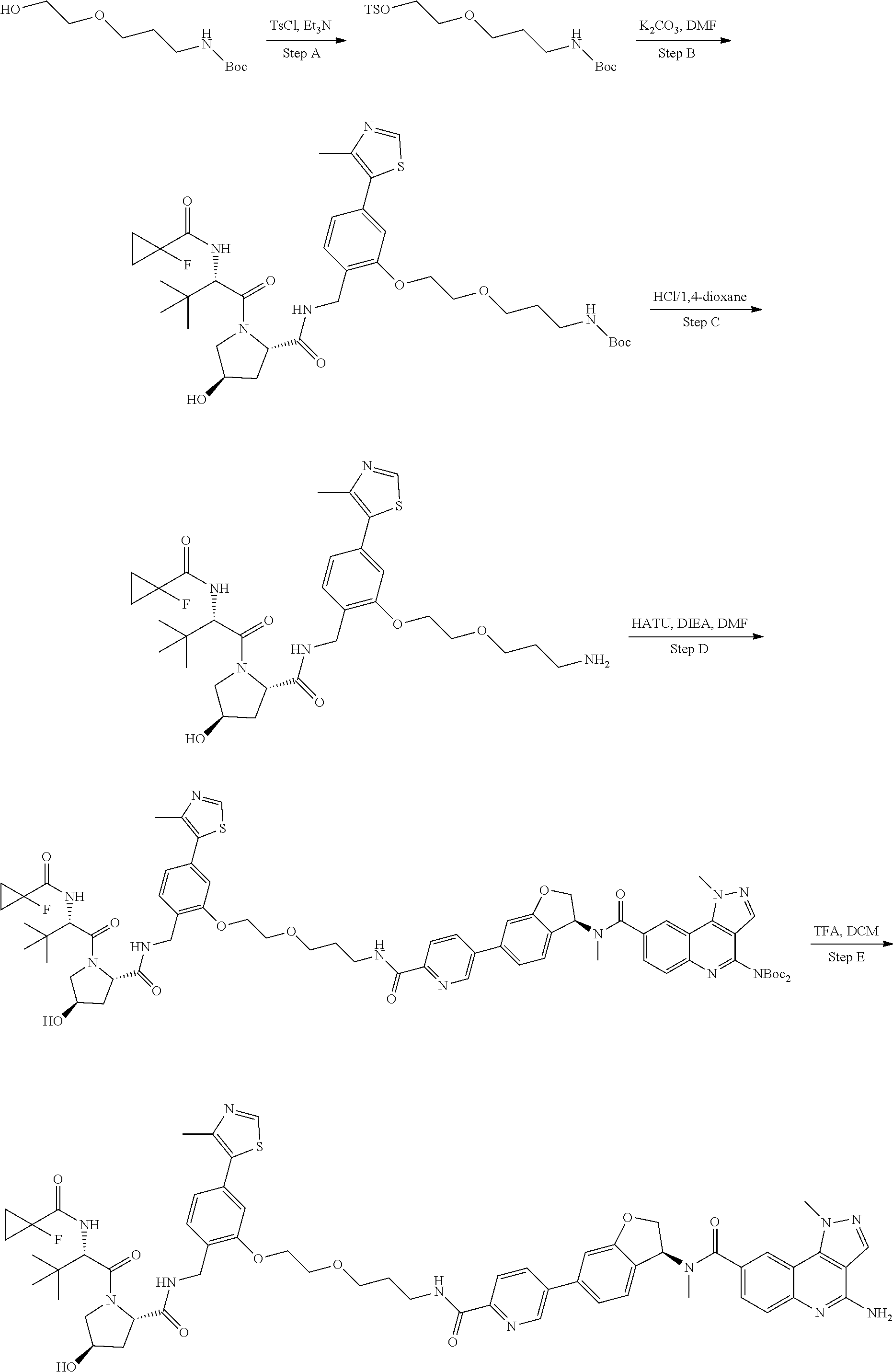

Step A: 2-(3-((tert-butoxycarbonyl)amino) propoxy)ethyl 4-methylbenzenesulfonate

To a solution of tert-butyl (3-(2-hydroxyethoxy) propyl)carbamate (200 mg, 0.9 mmol, 1.0 eq.) and TEA (0.4 mL, 2.7 mmol, 3.0 eq.) in MeCN (10 mL) was added TsCl (259 mg, 1.4 mmol, 1.5 eq.) and DMAP (222 mg, 1.8 mmol, 2.0 eq.). Then the mixture was stirred at room temperature for 3 hours. The resulting solution was diluted with H₂O (20 mL) and extracted with EtOAc (20 mL×3). The combined organic layers were washed with brine, dried over anhydrous Na₂SO₄ and concentrated under reduced pressure. The residue was purified on silica gel eluting (0 ~2% MeOH in DCM) to afford 2-(3-((tert-butoxycarbonyl)amino) propoxy)ethyl 4-methylbenzenesulfonate (140 mg, 41% yield) as a white solid.

LC-MS (ESI): mass calcd, for C17H27NO₆S, 373.2; m/z found, 374.1 [M+H]⁺.

Step B: tert-butyl (3-(2-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) propyl)carbamate

To a solution of 2-(3-((tert-butoxycarbonyl)amino) propoxy)ethyl 4-methylbenzenesulfonate (40 mg, 0.1 mmol, 1.2 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (50 mg, 0.09 mmol, 1.0 eq.) in DMF (2 mL) was added K₂CO₃ (52 mg, 0.4 mmol, 4.0 eq.). Then the mixture was stirred at 50° C. for 6 hours. The resulting solution was diluted with H₂O (20 mL) and extracted with EtOAc (20 mL×3). The combined organic layers were washed with brine, dried over anhydrous Na₂SO₄ and concentrated under reduced pressure. The residue was purified on silica gel eluting (0~7% MeOH in DCM) to afford tert-butyl (3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) propyl)carbamate (50 mg, 88% yield) as a colorless oil.

LC-MS (ESI): mass calcd, for C₃₆H₅₂FN₅O₈S, 733.4; m/z found, 734 [M+H]⁺.

### Steps C-E

Example A23 was synthesized following the same procedures as described in Example A1 (10 mg, 21% yield over 3 steps) as a white solid. LC-MS (ESI): mass calcd. for C₅₈H₆₄FN₁₁O₉S, 1109.5; m/z found, 1110.6 [M+H]⁺.

### Example A24. 4-amino-N—((S)-6-(6-((5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)pentyl)-carbamoyl)pyridin-3-yl)-2,3-dihydrobenzofuran-3-yl)-N,1-dimethyl-1H-pyrazolo[4,3-c]quinoline-8-carboxamide

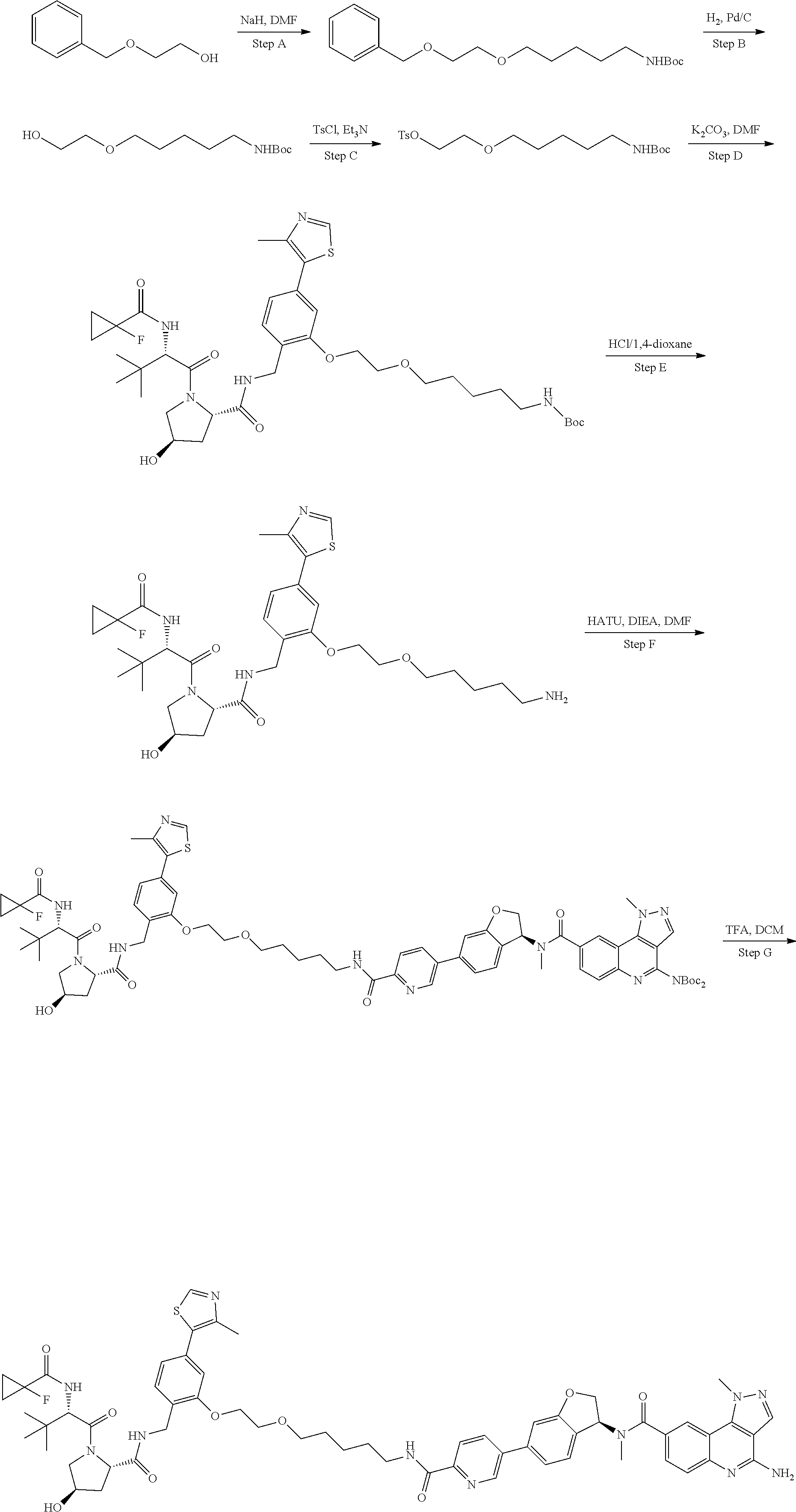

Step A: tert-butyl (5-(2-(benzyloxy)ethoxy)pentyl)carbamate

To a solution of 2-(benzyloxy) ethan-1-ol (1 g, 6.6 mmol, 1.0 eq.) in DMF (20 mL) was added NaH (400 mg, 9.85 mmol, 1.5 eq., 60%) was added at 0° C. After 0.5 hour, tert-butyl (5-bromopentyl)carbamate (2.1 g, 7.88 mmol, 1.2 eq.) was added at 0° C. Then the mixture was stirred at room temperature for 2 hours. The mixture was poured into water and extracted with EtOAc (20 mL×3). The combined organic layers were washed with brine, dried over anhydrous Na₂SO₄ and concentrated under reduced pressure. The residue was purified on silica gel eluting (0 ~ 25% EtOAc in PE) to afford tert-butyl (5-(2-(benzyloxy)ethoxy)pentyl)carbamate (200 mg, 9% yield) as a yellow solid.

LC-MS (ESI): mass calcd, for C₁₉H₃₁NO₄, 377.2; m/z found, 378.1 [M+H]⁺.

Step B: tert-butyl (5-(2-hydroxyethoxy)pentyl)carbamate

A mixture of Example tert-butyl (5-(2-(benzyloxy)ethoxy)pentyl)carbamate (200 mg, 0.59 mmol), Pd/C(50% water, 30 mg) in MeOH (7 mL) was stirred under Hz at room temperature for 16 hours. The mixture was filtered and concentrated in vacuo to give tert-butyl (5-(2-hydroxyethoxy)pentyl)carbamate (130 mg, 89% yield) as a yellow solid.

LC-MS (ESI): mass calcd, for C₁₂H₂₅NO₄, 247.2; m/z found, 248.3 [M+H]⁺.

Step C: 2-((5-((tert-butoxycarbonyl)amino)pentyl)oxy)ethyl 4-methylbenzenesulfonate

To a solution of tert-butyl (5-(2-hydroxyethoxy)pentyl)carbamate (130 mg, 0.5 mmol, 1.0 eq.) and TEA (0.2 mL, 1.6 mmol, 3.0 eq.) in MeCN (10 mL) was added TsCl (149 mg, 0.8 mmol, 1.5 eq.) and DMAP (128 mg, 1.1 mmol, 2.0 eq.). Then the mixture was stirred at room temperature for 3 hours. The mixture was poured into water and extracted with EtOAc (20 mL×3). The combined organic layers were washed with brine, dried over anhydrous Na₂SO₄ and concentrated under reduced pressure. The residue was purified on silica gel eluting (0 ~ 2% MeOH in DCM) to afford 2-((5-((tert-butoxycarbonyl)amino)pentyl)oxy)ethyl 4-methylbenzenesulfonate (120 mg, 57% yield) as a white solid.

LC-MS (ESI): mass calcd, for C₁₉H₃₁NO₆S, 401.2; m/z found, 402.3 [M+H]⁺.

Step D: tert-butyl (5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)pentyl)carbamate

To a solution of 2-((5-((tert-butoxycarbonyl)amino)pentyl)oxy)ethyl 4-methylbenzenesulfonate (40 mg, 0.1 mmol, 1.1 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (50 mg, 0.1 mmol, 1.0 eq.) in DMF (2 mL) was added K₂CO₃ (52 mg, 0.4 mmol, 4.0 eq.). Then the mixture was stirred at 50° C. for 6 hours. The mixture was poured into water and extracted with EtOAc (20 mL×3). The combined organic layers were washed with brine, dried over anhydrous Na₂SO₄ and concentrated under reduced pressure. The residue was purified on silica gel eluting (0~7% MeOH in DCM) to afford tert-butyl (5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)pentyl) carbamate (30 mg, 49% yield) as a colorless oil.

LC-MS (ESI): mass calcd, for C₃₈H₅₆FN₅O₈S, 761.4; m/z found, 762.3 [M+H]⁺.

### Steps E-G

Example A24 was synthesized following the same procedures as described in Example A1 (14.8 mg, 25% yield over 3 steps) as a white solid. LC-MS (ESI): mass calcd. for C₆₀H₆₈FN₁₁O₉S, 1137.5; m/z found, 1138.4 [M+H]⁺.

### Example A25. 4-amino-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)(methyl)carbamoyl)pyridin-3-yl)-2,3-dihydrobenzofuran-3-yl)-N,1-dimethyl-1H-pyrazolo[4,3-c]quinoline-8-carboxamide

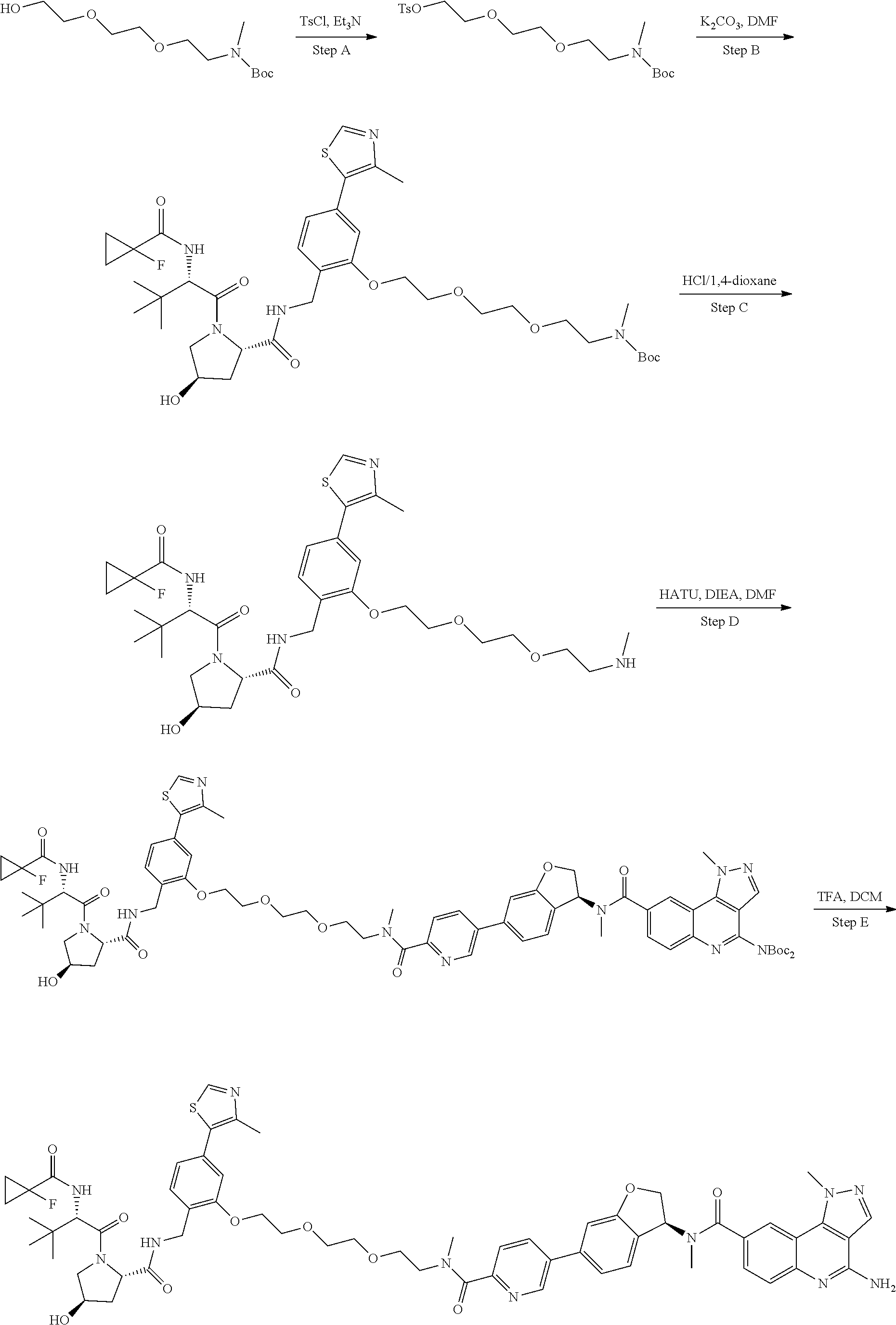

Step A: 2,2,5-trimethyl-4-oxo-3,8,11-trioxa-5-azatridecan-13-yl 4-methylbenzenesulfonate

To a solution of tert-butyl(2-(2-(2-hydroxyethoxy)ethoxy)ethyl)(methyl)carbamate (150 mg, 570 μmol, 1.0 eq.), Et₃N (120 mg, 1.2 mmol, 2.0 eq.) in DCM (5 mL) was added TsCl (163 mg, 854 μmol, 1.5 eq.). The mixture was stirred for 3 hours. The resulting solution was diluted with H₂O (20 mL) and extracted with DCM (20 mL×3). The combined organic layers were washed with brine (20 mL×2), dried over Na₂SO₄ and concentrated under reduced pressure, the residue was purified by flash column chromatography on silica gel (eluent, 0~20% EtOAc in PE) to give 2,2,5-trimethyl-4-oxo-3,8,11-trioxa-5-azatridecan-13-yl 4-methylbenzenesulfonate (150 mg, 63% yield) as a yellow oil.

LC-MS (ESI): mass calcd, for C₁₉H₃₁NO₇S, 417.18; m/z found, 418.2 [M+H]⁺.

Step B: tert-butyl(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)(methyl)carbamate

To a solution of 2,2,5-trimethyl-4-oxo-3,8,11-trioxa-5-azatridecan-13-yl 4-methylbenzenesulfonate (55 mg, 132 μmol, 1.3 eq.), (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (50 mg, 110 μmol, 1.0 eq.) in DMF (5 mL) was added K₂CO₃ (30 mg, 220 μmol, 2.0 eq.). The reaction mixture was stirred overnight at 55° C. The resulting solution was diluted with H₂O (20 mL) and extracted with EtOAc (20 mL×3). The combined organic layers were washed with brine, dried over anhydrous Na₂SO₄ and concentrated under reduced pressure. The residue was purified by flash chromatographer (eluent, 0~4% MeOH in DCM) to give tert-butyl(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)(methyl)carbamate (80 mg, 78% yield) as a yellow solid.

LC-MS (ESI): mass calcd, for C₃₈H₅₆FN₅O₉S, 777.38; m/z found, 778.4 [M+H]⁺.

### Steps C-E

Example A25 was synthesized following the same procedures as described in Example A1 (6 mg, 7% yield over 3 steps) as a white solid. LC-MS (ESI): mass calcd, for C₆₀H₆₈FN₁₁O₁₀S, 1153.5; m/z found, 1154.3 [M+H]⁺.

### Example A26. 4-amino-N—((S)-6-(6-((2-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentyl)oxy)ethyl)carbamoyl)pyridin-3-yl)-2,3-dihydrobenzo-furan-3-yl)-N,1-dimethyl-1H-pyrazolo[4,3-c]quinoline-8-carboxamide

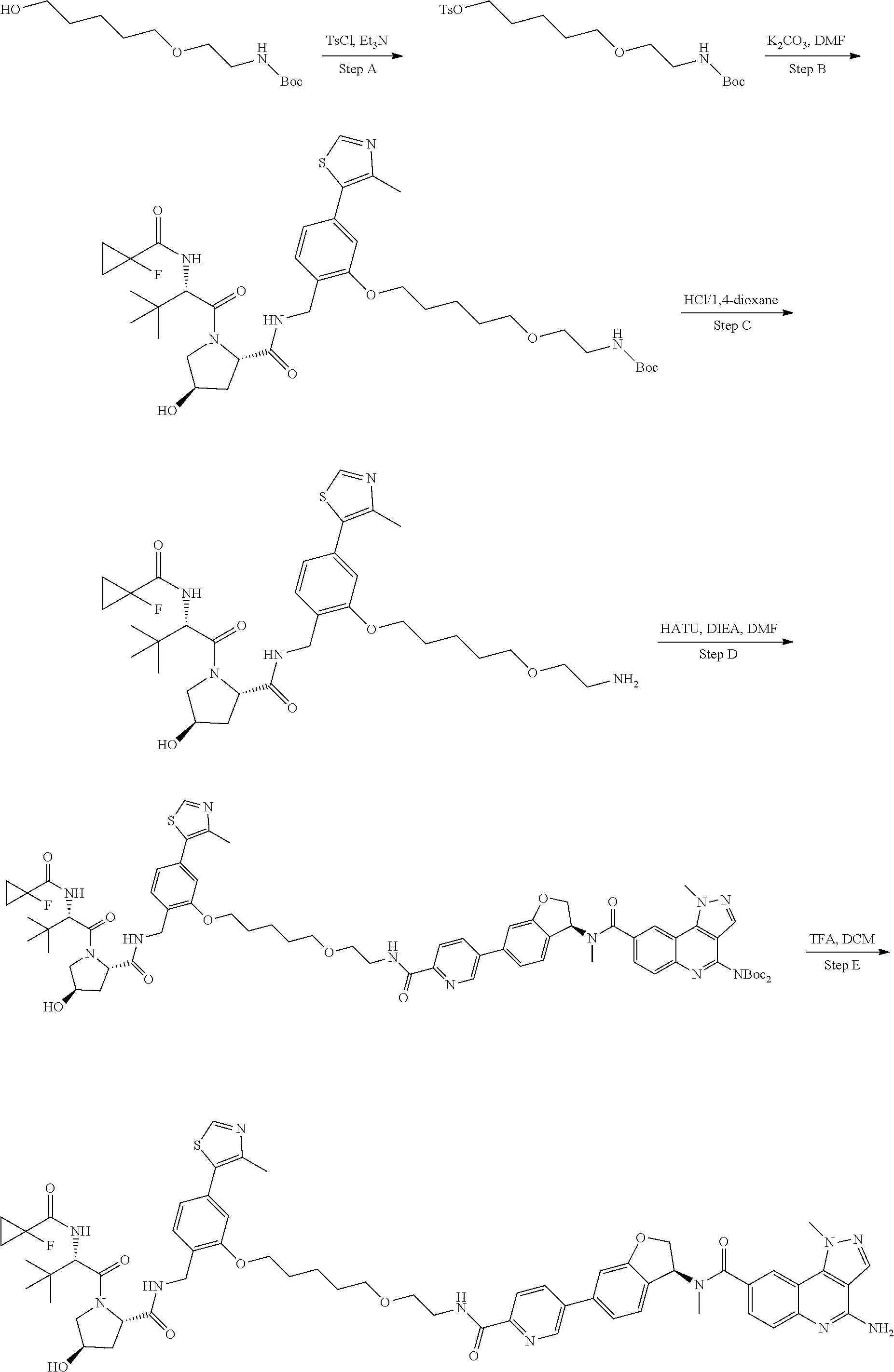

Step A: 5-(2-((tert-butoxycarbonyl)amino)ethoxy) pentyl 4-methylbenzenesulfonate

To a solution of tert-butyl(2-((5-hydroxypentyl)oxy)ethyl)carbamate (150 mg, 0.6 mmol, 1.0 eq.), Et₃N (120 mg, 1.2 mmol, 2.0 eq.) in DCM (5 mL) was added TsCl (171 mg, 0.9 mmol, 1.5 eq.). The mixture was stirred for 3 hours. The resulting solution was diluted with H₂O (20 mL), extracted with DCM (20 mL×3). The combined organic layers were washed with brine (20 mL×2), dried over Na₂SO₄ and concentrated under reduced pressure, the residue was purified by flash column chromatography on silica gel (eluent, 0~20% EtOAc in PE) to give 5-(2-((tert-butoxycarbonyl)amino)ethoxy) pentyl 4-methylbenzenesulfonate (150 mg, 62% yield) as a yellow oil.

LC-MS (ESI): mass calcd, for C19H31NO₆S, 401.19; m/z found, 402.1 [M+H]⁺.

Step B: tert-butyl(2-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentyl)oxy)ethyl)carbamate

To a solution of 5-(2-((tert-butoxycarbonyl)amino)ethoxy) pentyl 4-methylbenzenesulfonate (55 mg, 137 μmol, 1.3 eq.), (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (52 mg, 105 μmol, 1.0 eq.) in DMF (5 mL) was added K₂CO₃ (29 mg, 210 μmol, 2.0 eq.). The reaction mixture was stirred overnight at 55° C. The resulting solution was diluted with H₂O (20 ml) and extracted with EtOAc (20 mL×3). The combined organic layers were washed with brine, dried over anhydrous Na₂SO₄ and concentrated under reduced pressure. The residue was purified by flash chromatographer (eluent, 0~4% MeOH in DCM) to give tert-butyl(2-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentyl)oxy)ethyl)carbamate (50 mg, 67% yield) as a yellow solid.

LC-MS (ESI): mass calcd, for C₃₈H₅₆FN₅O₈S, 761.38; m/z found, 762.3 [M+H]⁺.

### Step C-E

Example A26 was synthesized following the same procedures as described in Example A1 (5 mg, 4% yield over 3 steps) as a white solid. LC-MS (ESI): mass calcd, for C₆₀H₆₈FN₁₁O₉S, 1137.49; m/z found, 1138.4 [M+H]⁺.

### Example A27. 4-amino-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzo furan-3-yl)-N-methyl-1,3-dihydrofuro[3,4-c][1,7]naphthyridine-8-carboxamide

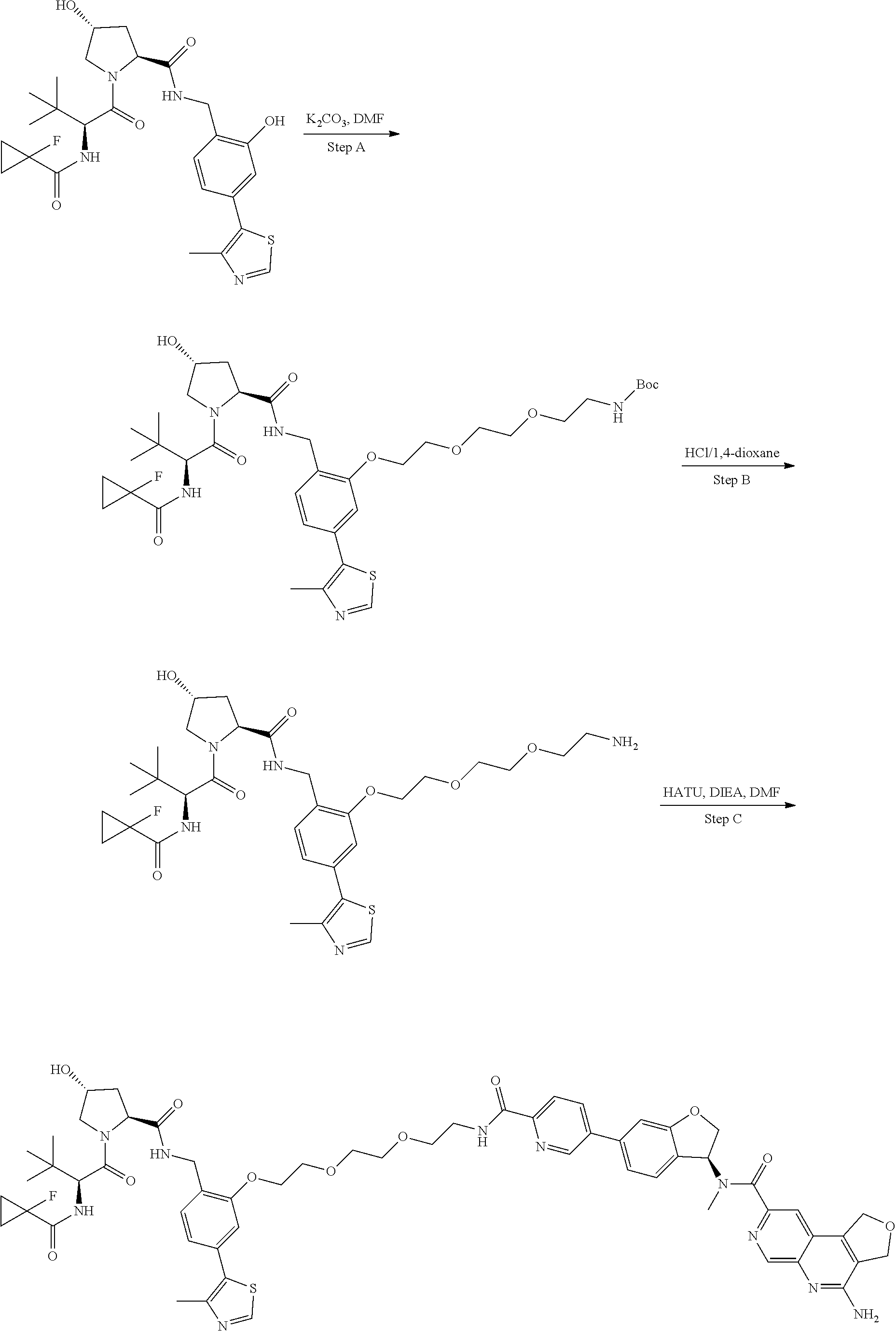 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3cc4c5c(c(N)nc4cn3)COC5)cn2)c1]

Example A27 was synthesized following the same procedures as described in Example A9 (9.5 mg, 18% yield over 3 steps) as a white solid. LC-MS (ESI): mass calcd. for C₅₈H₆₅FN₁₀O₁₁S, 1128.4; m/z found, 1129.1 [M+H]⁺.

### Example A28. 4-amino-N—((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N,1-dimethyl-1H-pyrazolo[4,3-c]quinoline-8-carboxamide

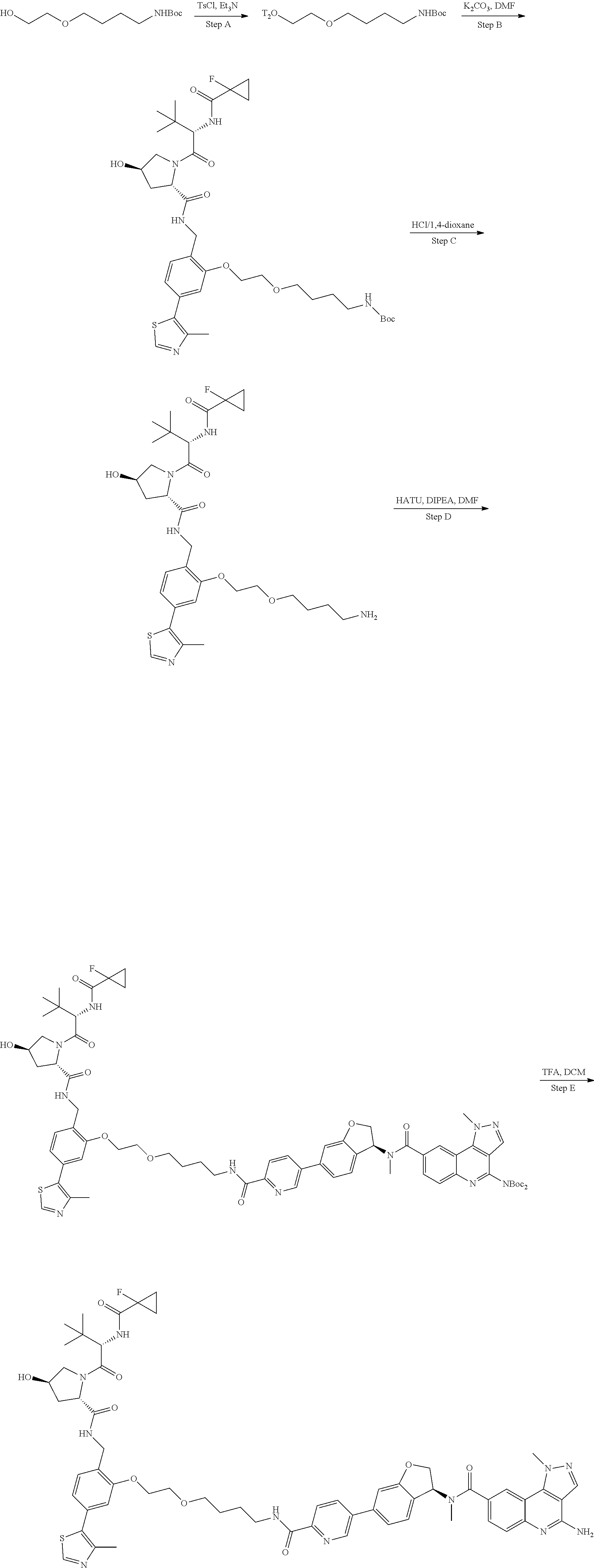

Step A: 2-(4-((tert-butoxycarbonyl)amino)butoxy)ethyl 4-methylbenzenesulfonate

To a solution of tert-butyl (4-(2-hydroxyethoxy)butyl)carbamate (250 mg, 1.07 mmol, 1.0 eq.) and TEA (0.6 mL,3.22 mmol, 3.0 eq.) in MeCN (10 mL) was added TsCl (305 mg, 1.61 mmol, 1.5 eq.), followed by DMAP (261 mg, 2.15 mmol, 2.0 eq.). Then the mixture was stirred at room temperature for 3 hours. The mixture was diluted with water and extracted three times with EtOAc (30 mL). The organic phase was dried over Na₂SO₄, filtered and concentrated in vacuum. The residue was purified on silica gel eluting (0 ~ 2% MeOH in DCM) to afford 2-(4-((tert-butoxycarbonyl)amino)butoxy)ethyl 4-methylbenzenesulfonate (200 mg, 48% yield) as a white solid.

LC-MS (ESI): mass calcd, for C18H19NO₆S, 387.2; m/z found, 388.1 [M+H]⁺.

Step B: tert-butyl (4-(2-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamate

To a solution of 2-(4-((tert-butoxycarbonyl)amino)butoxy)ethyl 4-methylbenzenesulfonate (47 mg, 0.12 mmol, 1.3 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (50 mg, 0.10 mmol, 1.0 eq.) in DMF (2 mL) was added K₂CO₃ (52 mg, 0.38 mmol, 4.0 eq.). Then the mixture was stirred at 50° C. for 6 hours. The mixture was diluted with water and extracted three times with EtOAc (20 mL). The organic phase was dried over Na₂SO₄, filtered and concentrated in vacuum. The residue was purified on silica gel eluting (0~6% MeOH in DCM) to afford tert-butyl (4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamate (60 mg, 85% yield) as a colorless oil.

LC-MS (ESI): mass calcd, for C₃₇H₅₄FN₅O₈S, 747.4; m/z found, 748.4 [M+H]⁺.

### Step C-E

Example A28 was synthesized following the same procedures as described in Example A1 (18 mg, 32% yield over 3 steps) as a white solid. LC-MS (ESI): mass calcd. for C₅₉H₆₆FN₁₁O₉S, 1123.47; m/z found, 1124.3 [M+H]⁺.

### Example A29. 4-amino-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-carbamoyl) pyridin-3-yl)-2,3-dihydrobenzofuran-3-yl)-N,1-dimethyl-1H-pyrazolo[4,3-c]quinoline-8-carboxamide

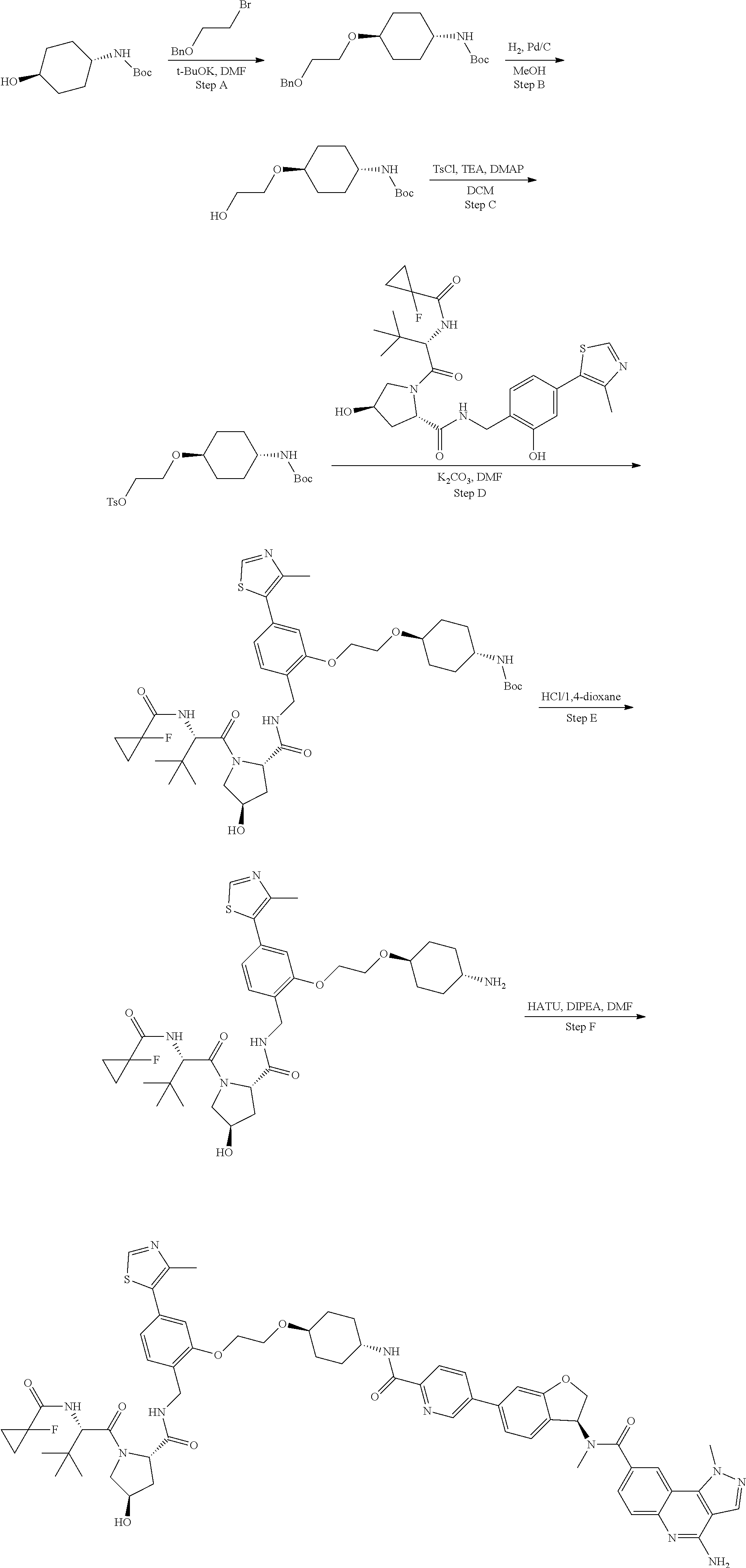

Step A: tert-butyl ((1r,4r)-4-(2-(benzyloxy)ethoxy)cyclohexyl)carbamate

To a solution of tert-butyl ((1r,4r)-4-hydroxycyclohexyl)carbamate (1.0 g, 4.6 mmol, 1.0 eq.) in THF (10 mL) was added 2-methylpropan-2-olate potassium (625 mg, 0.64 mL, 5.6 mmol, 1.2 eq.) at 0° C. and stirred for 30 min under 0° C. Then ((2-bromoethoxy)methyl) benzene (1 g, 4.64 mmol, 1.0 eq.) was added at 0° C. and the mixture stirred at room temperature for 16 hours. The mixture was poured into water and extracted with EtOAc (30 mL×3). The combined organic layers were washed with brine, dried over anhydrous Na₂SO₄ and concentrated under reduced pressure. The residue was purified by flash chromatographer (eluent, 0~30% EtOAc in PE) to give the tert-butyl ((1r,4r)-4-(2-(benzyloxy)ethoxy)cyclohexyl)carbamate (100 mg, 6% yield) as a colorless oil.

LC-MS (ESI): mass calcd, for C₂₀H₃₁NO₄, 349.26; m/z found, 366.8 [M+H₂O]⁺.

Step B: tert-butyl ((1r,4r)-4-(2-hydroxyethoxy)cyclohexyl)carbamate

To a solution of tert-butyl ((1r,4r)-4-(2-(benzyloxy)ethoxy)cyclohexyl)carbamate (100 mg, 286 μmol, 1.0 eq.) in MeOH (2.0 mL) was added Pd/C(20 mg, 10% Wt) at room temperature under nitrogen atmosphere. Then was stirred at room temperature for 5 hours under H₂ balloon. The mixture was filtered and concentrated under reduced pressure to dryness to give the tert-butyl ((1r,4r)-4-(2-hydroxyethoxy)cyclohexyl)carbamate (60 mg, 81% yield) as a yellow oil.

LC-MS (ESI): mass calcd, for C₁₃H₂₅NO₄, 259.18; m/z found, 204.2 [M+H−56]⁺.

Step C: 2-(((1r,4r)-4-((tert-butoxycarbonyl)amino)cyclohexyl)oxy)ethyl4-methylbenzenesulfonate

To a mixture of tert-butyl ((1r,4r)-4-(2-hydroxyethoxy)cyclohexyl)carbamate (30.0 mg, 116 μmol, 1.0 eq.), TEA (35.1 mg, 48.4 μL, 347 μmol, 3.0 eq.) and DMAP (14.0 mg, 116 μmol, 1.0 eq.) in DCM (2 mL) was added TsCl (33 mg, 26.4 μL, 174 μmol, 1.5 eq.) at 0° C. Then the mixture was stirred at room temperature for 1 hour. The mixture was poured into water and extracted with EtOAc (20 mL×3). The combined organic layers were washed with brine, dried over anhydrous Na₂SO₄ and concentrated under reduced pressure to give the 2-(((1r,4r)-4-((tert-butoxycarbonyl)amino)cyclohexyl)oxy)ethyl 4-methylbenzenesulfonate (35 mg, 73% yield) as a yellow oil. The mixture was used for next step without further purification.

LC-MS (ESI): mass calcd, for C20H31NO₆S, 413.19; m/z found, 414.3 [M+H]⁺.

Step D: tert-butyl ((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamate

To a mixture of 2-(((1r,4r)-4-((tert-butoxycarbonyl)amino)cyclohexyl)oxy)ethyl 4-methylbenzenesulfonate (23.0 mg, 56.3 μmol, 1.0 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (30.0 mg, 56.3 μmol, 1.0 eq.) in DMF (2 mL) was added K₂CO₃ (23.4 mg, 169 μmol, 3.0 eq.) at room temperature. Then the mixture was stirred at 55° C. for 5 hours. The mixture was poured into water and extracted with EtOAc (30 mL×3). The combined organic layers were washed with brine, dried over anhydrous Na₂SO₄ and concentrated under reduced pressure. The residue was purified by flash chromatographer (eluent, 0~5% MeOH in DCM) to give the tert-butyl ((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamate (30 mg, 69% yield) as a colorless oil.

LC-MS (ESI): mass calcd, for C₂₀H₃₁NO₄, 773.38; m/z found, 774.6 [M+H]⁺.

### Steps E-F

Example A29 was synthesized following the same procedures as described in Example A1 (6.6 mg, 14.8% yield over 2 steps) as a white solid. LC-MS (ESI): mass calcd. for C₆₁H₆₈FN₁₁O₉S, 1149.49; m/z found, 1150.6 [M+H]⁺. ¹H NMR (400 MHz, DMSO-d₆) δ 8.98 (s, 1H), 8.92 (s, 1H), 8.54-8.45 (m, 2H), 8.39-8.30 (m, 2H), 8.27 (d, J=8.1 Hz, 1H), 8.08 (d, J=8.2 Hz, 1H), 7.75-7.63 (m, 2H), 7.62-7.55 (m, 1H), 7.43-7.37 (m, 2H), 7.34 (s, 1H), 7.29 (d, J=6.9 Hz, 1H), 7.05 (s, 1H), 6.97 (d, J=9.0 Hz, 1H), 5.17 (d, J=3.5 Hz, 1H), 4.87-4.68 (m, 2H), 4.59 (d, J=9.4 Hz, 1H), 4.52 (t, J=8.2 Hz, 1H), 4.44 (s, 3H), 4.38-4.34 (m, 1H), 4.30 (d, J=5.9 Hz, 1H), 4.26-4.20 (m, 1H), 4.19-4.13 (m, 2H), 3.87-3.76 (m, 3H), 3.69-3.58 (m, 2H), 3.11-3.05 (m, 1H), 2.72 (s, 3H), 2.47 (s, 3H), 2.12-2.01 (m, 3H), 1.97-1.90 (m, 1H), 1.89-1.80 (m, 2H), 1.56-1.44 (m, 2H), 1.42-1.28 (m, 4H), 1.27-1.13 (m, 5H), 0.95 (s, 9H).

### Example A30. 4-amino-N—((S)-6-(6-(4-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl) piperazine-1-carbonyl)pyridin-3-yl)-2,3-dihydrobenzofuran-3-yl)-N,1-dimethyl-1H-pyrazolo[4,3-c]quinoline-8-carboxamide

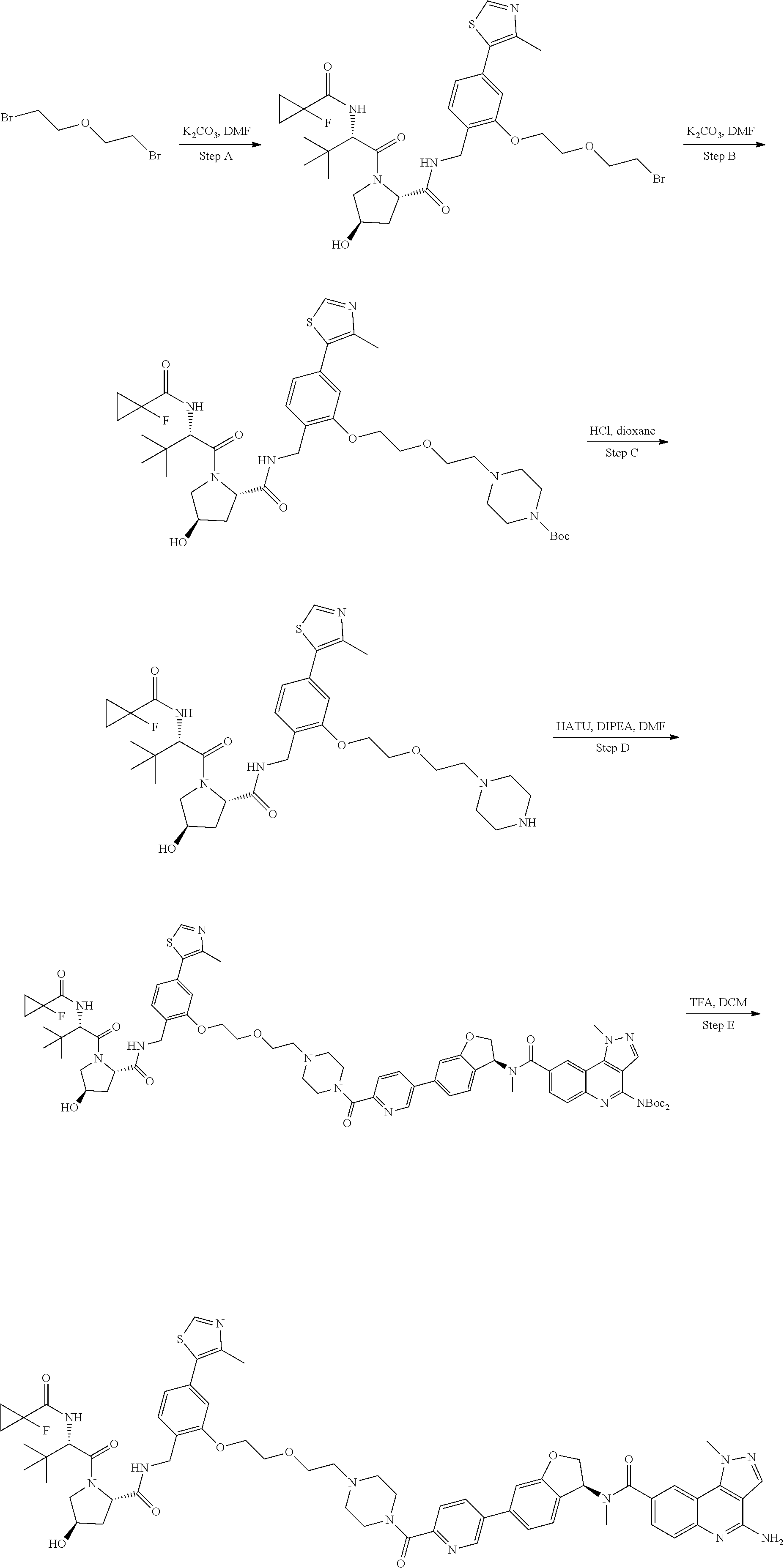

Example A30 was synthesized following the same procedures as described in Example A17 (2.8 mg, 4% yield over 5 steps) as a white solid. LC-MS (ESI): mass calcd. for C₆₁H₆₉FN₁₂O₉S, 1164.5; m/z found, 1165.3 [M+H]⁺.

### Example A31. N1-(6-amino-5-ethylpyridin-3-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-N2-methyloxalamide

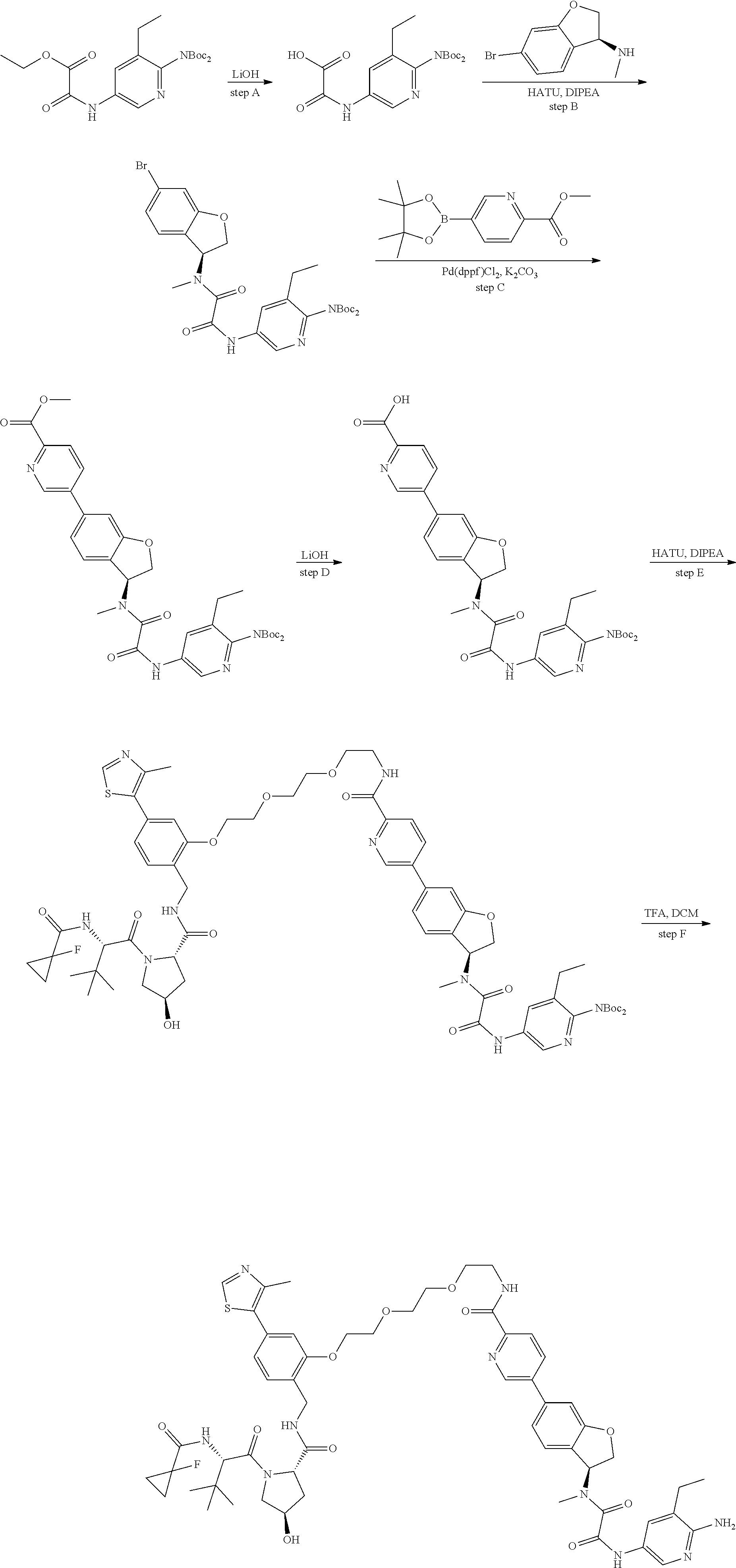

Step A: 2-(6-(bis(tert-butoxycarbonyl)amino)-5-ethylpyridin-3-yl)amino)-2-oxoacetic acid

To a solution of ethyl 2-((6-(bis(tert-butoxycarbonyl)amino)-5-ethylpyridin~3-yl)amino)-2-oxoacetate (200 mg, 457 μmol, 1.0 eq.) in Water (2 mL) and EtOH (5 mL) was added the solution of Lithium hydroxide monohydrate (21.1 mg, 503 μmol, 1.1 eq.) in Water (3.00 mL) at 0° C. The resulting mixture was stirred at 0° C. for 3 hours. The mixture was added IM hydrochloric acid to adjust pH=3-4, extracted with EtOAc (30 mL×2), dried over Na₂SO₄ and concentrated under reduced pressure to afford 2-((6-(bis(tert-butoxycarbonyl)amino)-5-ethylpyridin-3-yl)amino)-2-oxoacetic acid (150 mg, 80% yield) as light yellow solid.

LC-MS (ESI): mass calcd, for C₁₉H₂₇N₃O₇, 409.2; m/z found, 410.1 [M+H]⁺.

Step B: tert-butyl(S)-(5-(2-((6-bromo-2,3-dihydrobenzofuran-3-yl)(methyl)amino)-2-oxoacetamido)-3-ethylpyridin-2-yl)(tert-butoxycarbonyl)carbamate

A solution of 2-((6-(bis(tert-butoxycarbonyl)amino)-5-ethylpyridin-3-yl)amino)-2-oxoacetic acid (150 mg, 366 μmol, 1.0 eq.) and(S)-6-bromo-N-methyl-2,3-dihydrobenzofuran-3-amine (100 mg, 439 μmol, 1.2 eq.) in DMF (3.00 mL) was added HATU (209 mg, 549 μmol, 1.5 eq.) and DIEA (191 μL, 1.0 mmol, 3.0 eq.). The mixture was stirred at room temperature for 1 hour. The resulting solution was diluted with 100 mL of H₂O and extracted with EtOAc (20 mL×3). The combined organic layers were washed with brine, dried over Na₂SO₄ and concentrated under reduced pressure. The residue was purified by flash column chromatography on silica gel (100-200 mesh silica gel, 0-30% EtOAc in PE) to give tert-butyl(S)-(5-(2-((6-bromo-2,3-dihydrobenzofuran-3-yl)(methyl)amino)-2-oxoacetamido)-3-ethylpyridin-2-yl)(tert-butoxycarbonyl)carbamate (190 mg, 84% yield) as a colorless oil.

LC-MS (ESI): mass calcd, for C₂₈H₃₅BrN₄O₇, 618.2; m/z found, 619.2 [M+H]⁺.

Step C: methyl(S)-5-(3-(2-((6-(bis(tert-butoxycarbonyl)amino)-5-ethylpyridin-3-yl)amino)-N-methyl-2-oxoacetamido)-2,3-dihydrobenzofuran-6-yl)picolinate

A solution of tert-butyl(S)-(5-(2-((6-bromo-2,3-dihydrobenzofuran-3-yl)(methyl)amino)-2-oxoacetamido)-3-ethylpyridin-2-yl)(tert-butoxycarbonyl)carbamate (95 mg, 153 μmol, 1.0 eq.) and methyl 5-(4,4,5,5-tetramethyl-1,3,2-dioxaborolan-2-yl)picolinate (61 mg, 230 μmol, 1.5 eq.) in 1,4-Dioxane (2.0 mL) and Water (0.10 mL) was added K₂CO₃ (64 mg, 460 μmol, 3.0 eq.) and PdCl₂ (dppf)(11 mg, 15.3 μmol, 0.1 eq.). The mixture was stirred at 90° C. for 3 hours. The resulting solution was diluted with 100 mL of H₂O and extracted with EtOAc (20 mL×3). The combined organic layers were washed with brine, dried over Na₂SO₄ and concentrated under reduced pressure. The residue was purified by flash column chromatography on silica gel (100-200 mesh silica gel, 0-67% EtOAc in PE) to give methyl(S)-5-(3-(2-((6-(bis(tert-butoxycarbonyl)amino)-5-ethylpyridin-3-yl)amino)-N-methyl-2-oxoacetamido)-2,3-dihydrobenzofuran-6-yl)picolinate (85.0 mg, 82% yield) as a colorless oil.

LC-MS (ESI): mass calcd, for C₃₅H₄₁N₅O₉, 675.3; m/z found, 676.4 [M+H]⁺.

Step D: (S)-5-(3-(2-(6-(bis(tert-butoxycarbonyl)amino)-5-ethylpyridin-3-yl)amino)-N-methyl-2-oxoacetamido)-2,3-dihydrobenzofuran-6-yl)picolinic acid

A solution of methyl(S)-5-(3-(2-((6-(bis(tert-butoxycarbonyl)amino)-5-ethylpyridin-3-yl)amino)-N-methyl-2-oxoacetamido)-2,3-dihydrobenzofuran-6-yl)picolinate (42 mg, 62 μmol, 1.0 eq.) in THF (2 mL) and Water (1 mL) was added Lithium hydroxide monohydrate (5.22 mg, 124 μmol, 2.0 eq.). The mixture was stirred at room temperature for 1 hour. The resulting solution was concentrated under reduced pressure to give(S)-5-(3-(2-((6-(bis(tert-butoxycarbonyl)amino)-5-ethylpyridin-3-yl)amino)-N-methyl-2-oxoacetamido)-2,3-dihydrobenzofuran-6-yl) picolinic acid (42.0 mg, 94% yield) as a yellow solid. LC-MS (ESI): mass calcd, for C₃₄H₃₉N₅O₉, 661.3; m/z found, 662.2 [M+H]⁺.

### Step E-F

Example A31 was synthesized following the same procedures as described in Example A1 (5.0 mg, 17% yield over 2 steps) as a white solid. LC-MS (ESI): mass calcd. for C₅₆H₆₇FN₁₀O₁₁S, 1106.5; m/z found, 1107.9 [M+H]⁺.

### Example A32. 4-amino-N—((S)-6-(6-(9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carbonyl)pyridin-3-yl)-2,3-dihydrobenzofuran-3-yl)-N,1-dimethyl-1H-pyrazolo[4,3-c]quinoline-8-carboxamide

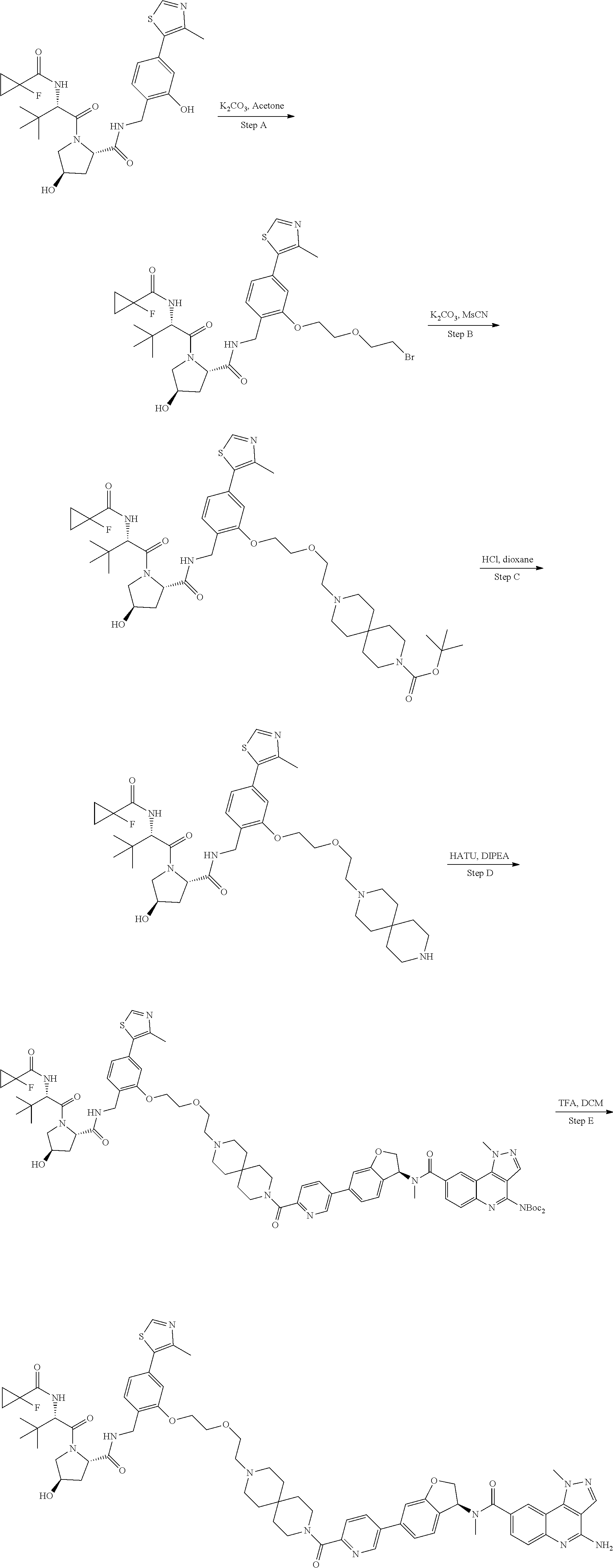

Step A: (2S,4R)—N-(2-(2-(2-bromoethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (100 mg, 0.19 mmol, 1.0 eq.), 1-bromo-2-(2-bromoethoxy) ethane (1 mL) in Acetone (5 mL) was added K₂CO₃ (54 mg, 0.38 mmol, 2.0 eq.). The mixture was stirred at 80° C. for 16 hours under N₂ atmosphere. The mixture was diluted with water (30 mL) and extracted with DCM (20 mL×3). The combined organic layers were washed with brine, dried over anhydrous Na₂SO₄ and concentrated under reduced pressure. The residue was purified by flash chromatographer (eluent, 0~4% MeOH in DCM) to give (2S,4R)—N-(2-(2-(2-bromoethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (80 mg, 62% yield) as a white solid.

LC-MS (ESI): mass calcd, for C₃₀H₄₀BrFN₄O₆S, 682,68; m/z found, 683,7 [M+H]⁺.

Step B: tert-butyl 9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carboxylate

To a solution of (2S,4R)—N-(2-(2-(2-bromoethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (80 mg, 0.12 mmol, 1.0 eq.), tert-butyl 3,9-diazaspiro[5.5]undecane-3-carboxylate (40 mg, 0.16 mmol, 1.3 eq.) in MeCN (8 mL) was added K₂CO₃ (33 mg, 0.24 mmol, 2.0 eq.). The reaction mixture was stirred overnight at 80° C. The mixture was poured into water and extracted with EtOAc (20 mL×3). The combined organic layers were washed with brine, dried over anhydrous Na₂SO₄ and concentrated under reduced pressure. The residue was purified by flash chromatographer (eluent, 0~10% MeOH in DCM) to give tert-butyl 9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carboxylate (40 mg, 53% yield) as a white solid.

LC-MS (ESI): mass calcd, for C₄₄H₆₅FN₆O₈S, 856.46; m/z found, 857.5 [M+H]⁺.

Step C: (2S,4R)—N-(2-(2-(2-(3,9-diazaspiro[5.5]undecan-3-yl)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of tert-butyl 9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carboxylate (40 mg, 0.05 mmol) in Dioxane (2 mL) was added HCl in Dioxane (2 mL, 4 mol/L) at 0° C. The reaction mixture was stirred for 1 hour at room temperature under N₂ atmosphere. The mixture was concentrated by vacuum to give (2S,4R)—N-(2-(2-(2-(3,9-diazaspiro[5.5]undecan-3-yl)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30 mg, 80% yield) as a white solid.

LC-MS (ESI): mass calcd, for C₃₉H₅₇FN₆O₆S, 756.40; m/z found, 757.5 [M+H]⁺.

Step D: tert-butyl (tert-butoxycarbonyl)(8-(((S)-6-(6-(9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carbonyl)pyridin-3-yl)-2,3-dihydrobenzofuran-3-yl)(methyl)carbamoyl)-1-methyl-1H-pyrazolo[4,3-c]quinolin-4-yl)carbamate

To a solution of (2S,4R)—N-(2-(2-(2-(3,9-diazaspiro[5.5]undecan-3-yl)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30 mg, 0.04 mmol, 1.0 eq.), (S)-5-(3-(4-(bis(tert-butoxycarbonyl)amino)-N,1-dimethyl-1H-pyrazolo[4,3c]quinoline-8-carboxamido)-2,3-dihydrobenzofuran-6-yl)picolinic acid (20 mg, 0.04 mmol, 1.0 eq.) and HATU (30 mg, 0.08 mmol, 2.0 eq.) in DMF (5 mL) was added Et₃N(12 mg, 0.12 mmol, 3.0 eq.) at 0° C. The reaction mixture was stirred at 0° C. for 30 mins under N₂ atmosphere. The mixture was diluted with water (30 mL) and extracted with EtOAc (20 mL×3). The combined organic layers were washed with brine, dried over anhydrous Na₂SO₄ and concentrated under reduced pressure. The residue was purified by flash chromatographer (eluent, 0~10% MeOH in DCM) to give tert-butyl (tert-butoxycarbonyl)(8-(((S)-6-(6-(9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carbonyl)pyridin-3-yl)-2,3-dihydrobenzofuran-3-yl)(methyl)carbamoyl)-1-methyl-1H-pyrazolo[4,3-c]quinolin-4-yl)carbamate (20 mg, 50% yield) as a white solid.

LC-MS (ESI): mass calcd, for C₇₆H₉₃FN₁₂O₁₃S, 1432.67; m/z found, 1433.6 [M+H]⁺.

Step E: 4-amino-N—(S)-Jun. 16, 2019-(2-(2-(2-(25, 4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carbonyl)pyridin-3-yl)-2,3-dihydrobenzofuran-3-yl)-N, 1-dimethyl-1H-pyrazolo[4,3-c]quinoline-8-carboxamide

To a solution of tert-butyl (tert-butoxycarbonyl)(8-(((S)-6-(6-(9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspiro[5.5]undecane-3-carbonyl)pyridin-3-yl)-2,3-dihydrobenzofuran-3-yl)(methyl)carbamoyl)-1-methyl-1H-pyrazolo[4,3-c]quinolin-4-yl)carbamate (20 mg, 0.01 mmol, 1.0 eq.) in dioxane (3 mL) was added TFA (1 mL) under N₂ atmosphere. The residue was purified by prep-HPLC(C18, 20~95%, MeCN in H₂O with 0.1% HCOOH) to give 4-amino-N—((S)-6-(6-(9-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethyl)-3,9-diazaspirocarbonyl) pyridin-3-yl)-2,3-dihydrobenzofuran-3-yl)-N,1-dimethyl-1H-pyrazolo[4,3-c]quinoline-8-carboxamide (6 mg, 40% yield) as a white solid. LC-MS (ESI): mass calcd, for C₆₆H₇₇FN₁₂O₉S, 1232.56; m/z found, 1233.4 [M+H]⁺.

### Example A33. N1-(6-amino-5-ethylpyridin-3-yl)-N2-((S)-6-(6-((8-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-octyl)carbamoyl)pyridin-3-yl)-2,3-dihydrobenzofuran-3-yl)-N2-methyloxalamide

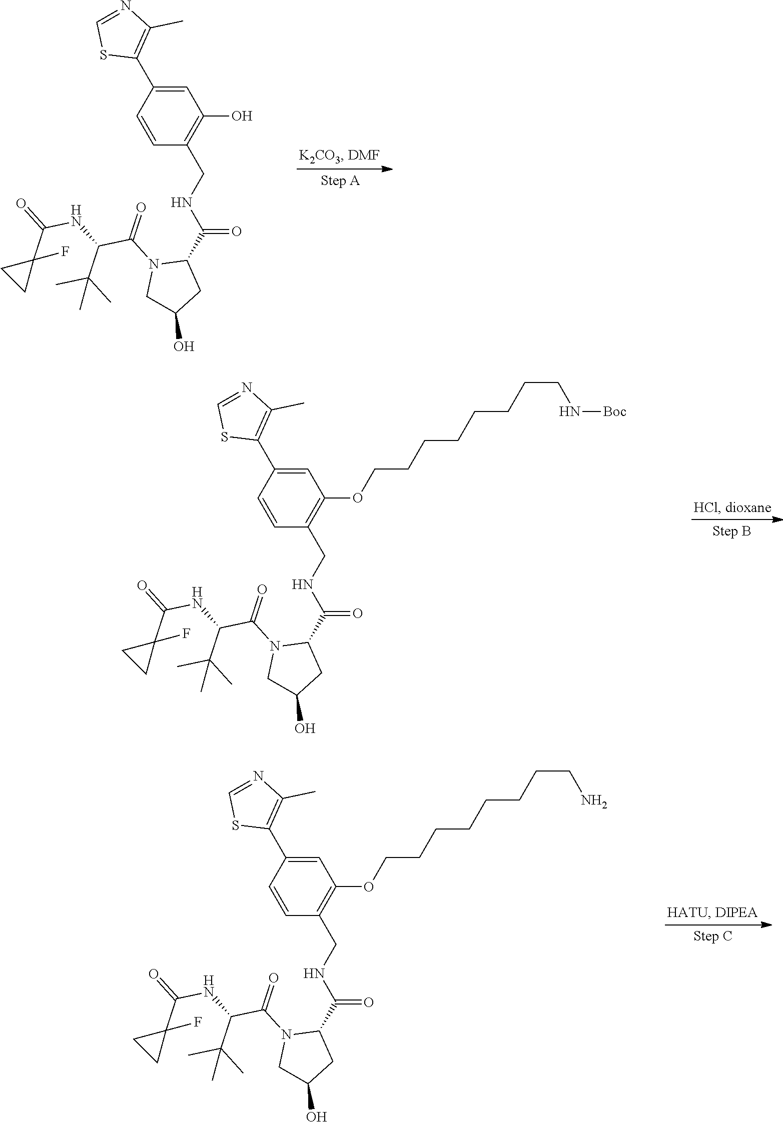 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCCN)c1]

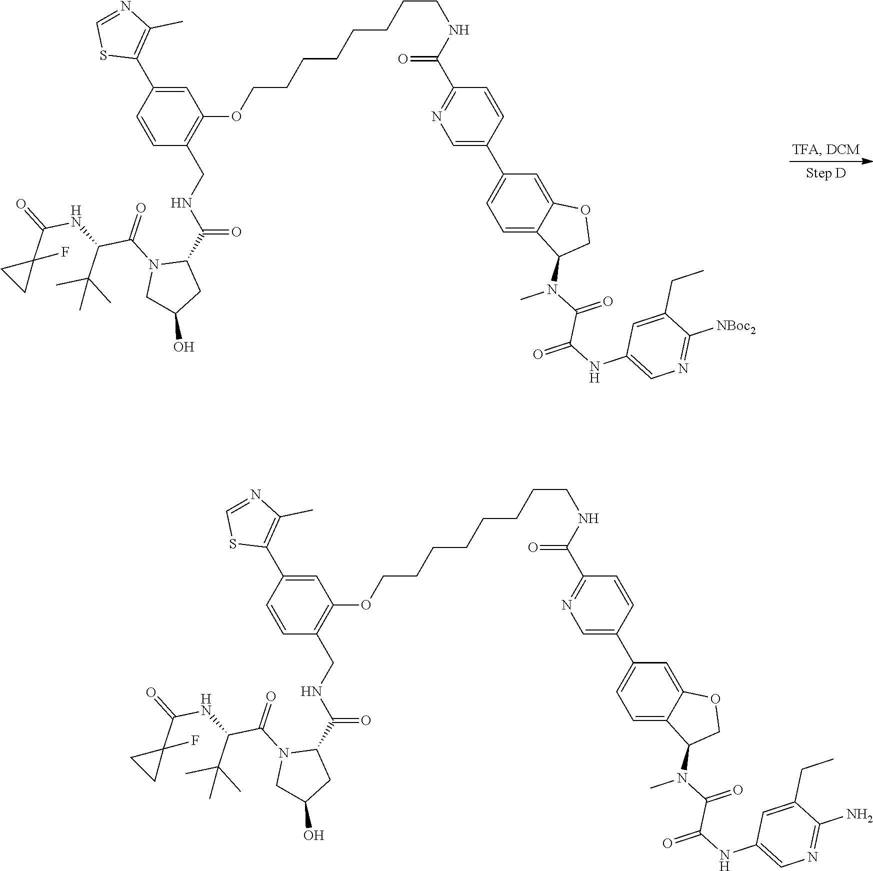

Example A33 was synthesized following the same procedures as described in Example A31 (3.5 mg, 5% yield over 4 steps) as a white solid. LC-MS (ESI): mass calcd. for C₅₈H₇₁FN₁₀O₉S, 1102.5; m/z found, 1103.4 [M+H]⁺.

### Example A34. N1-(6-amino-5-methylpyridin-3-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-N2-methyloxalamide

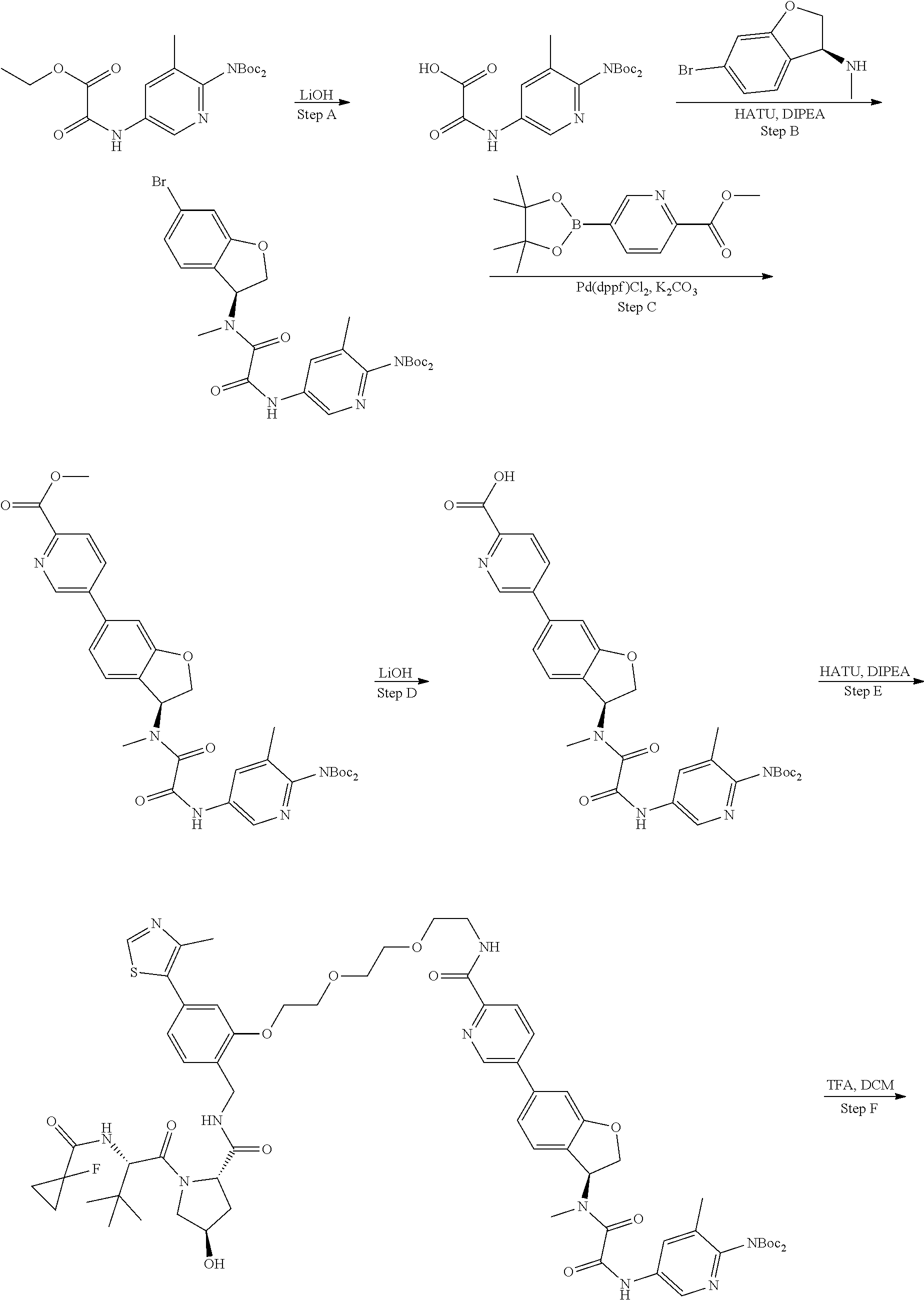

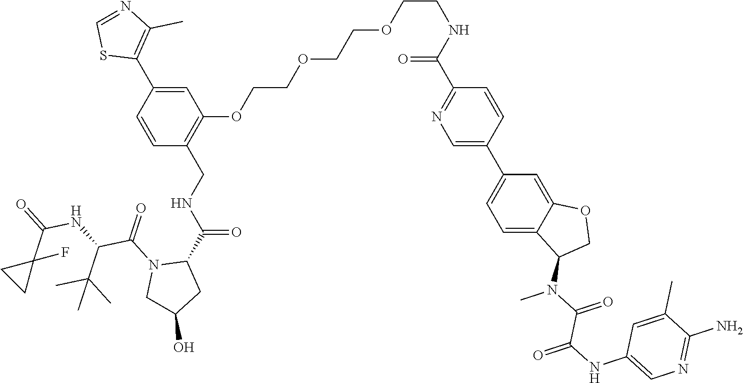 [SMILES: Cc1cc(NC(=O)C(=O)N(C)[C@@H]2COc3cc(-c4ccc(C(=O)NCCOCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N]

Example A34 was synthesized following the same procedures as described in Example A31 (9.0 mg, 14% yield over 6 steps) as a white solid. LC-MS (ESI): mass calcd. for C₅₅H₆₅FN₁₀O₁₁S, 1092.5; m/z found, 1093.2 [M+H]⁺. ¹H NMR (400 MHz, DMSO-d₆) δ 10.54 (d, J=62.6 Hz, 1H), 8.93 (d, J=15.8 Hz, 2H), 8.71 (t, J=5.6 Hz, 1H), 8.48 (t, J=5.7 Hz, 1H), 8.26 (d, J=8.3 Hz, 1H), 8.10-8.02 (m, 2H), 7.58-7.27 (m, 6H), 7.01 (s, 1H), 6.95 (d, J=7.6 Hz, 1H), 6.27-5.84 (m, 1H), 5.66 (s, 2H), 5.17 (d, J=3.5 Hz, 1H), 4.75 (d, J=12.7 Hz, 1H), 4.61 (dd, J=15.8, 7.3 Hz, 2H), 4.51 (t, J=8.2 Hz, 1H), 4.34 (s, 1H), 4.28 (d, J=5.9 Hz, 1H), 4.23 (d, J=5.5 Hz, 1H), 4.17 (s, 2H), 3.82-3.79 (m, 2H), 3.64 (d, J=5.4 Hz, 4H), 3.59 (d, J=5.5 Hz, 4H), 3.50 (s, 2H), 2.73 (s, 1H), 2.59 (s, 2H), 2.43 (s, 3H), 2.09 (d, J=13.4 Hz, 1H), 2.05 (d, J=3.5 Hz, 3H), 1.91 (t, J=8.8 Hz, 1H), 1.36 (dd, J=18.4, 9.7 Hz, 2H), 1.21 (d, J=8.5 Hz, 2H), 0.95 (s, 9H).

### Example A35. 4-amino-N—((S)-6-(6-((2-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentyl)(methyl)amino)ethyl)carbamoyl)pyridin-3-yl)-2,3-dihydrobenzofuran-3-yl)-N,1-dimethyl-1H-pyrazolo[4,3-c]quinoline-8-carboxamide

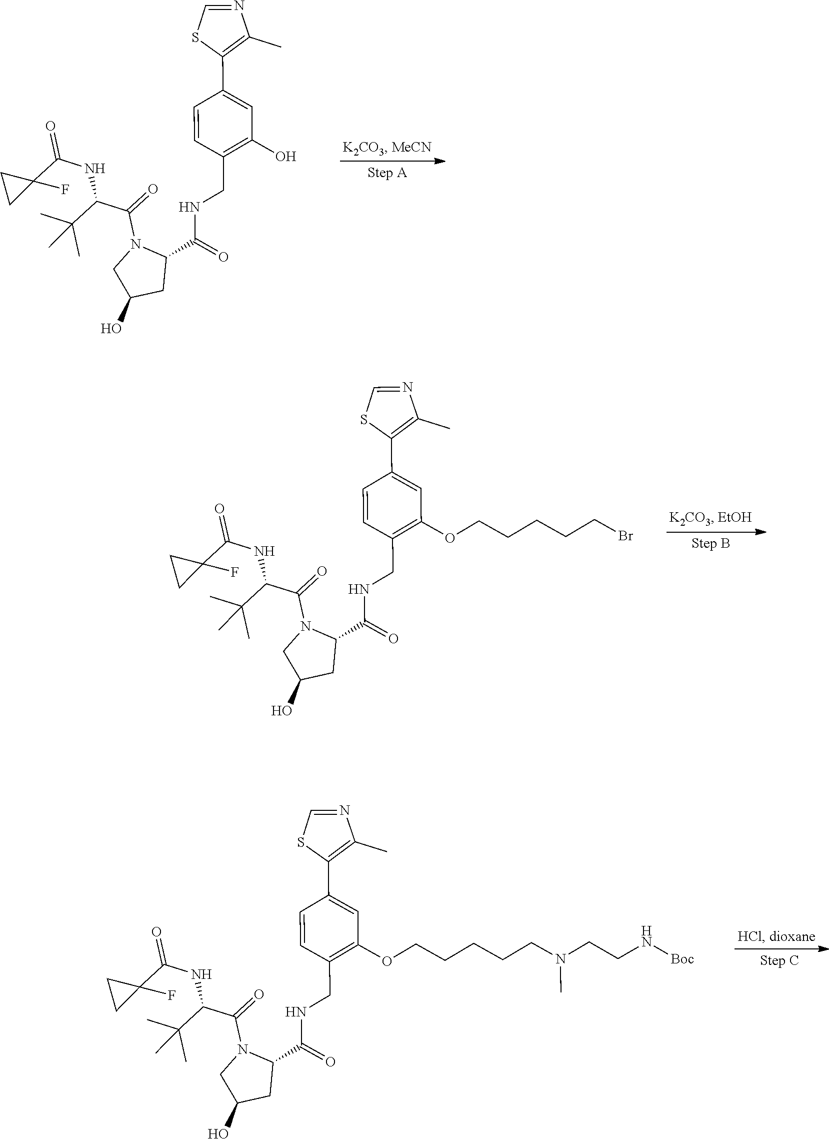 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCBr)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCN(C)CCNC(=O)OC(C)(C)C)c1]

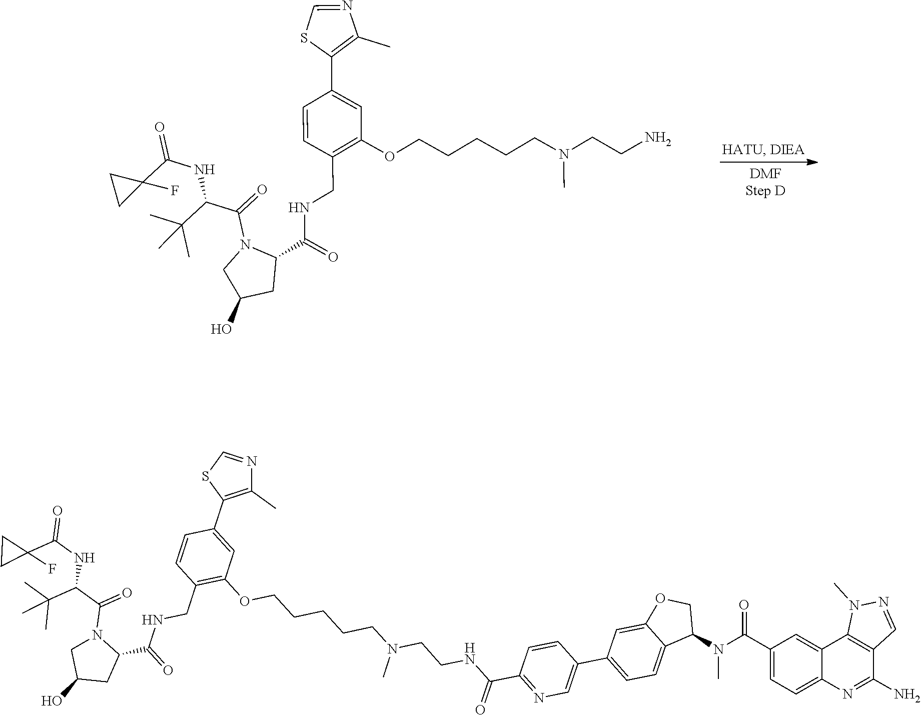 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCN(C)CCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCN(C)CCNC(=O)c2ccc(-c3ccc4c(c3)OC[C@H]4N(C)C(=O)c3ccc4nc(N)c5cnn(C)c5c4c3)cn2)c1]

Step A: (2S,4R)—N-(2-((5-bromopentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrroli-dine-2-carboxamide (40 mg, 0.08 mmol, 1.0 eq.) and1,5-dibromopentane (0.5 mL) in Acetone (5 mL) was added K₂CO₃ (35 mg, 0.25 mmol, 3.0 eq.). The mixture was stirred at 80° C. for 16 hours under N2 atmosphere. The mixture was diluted with water (30 mL) and extracted with DCM (20 mL×3). The combined organic layers were washed with brine, dried over anhydrous Na₂SO₄ and concentrated under reduced pressure. The residue was purified by flash chromatographer (eluent, 0~4% MeOH in DCM) to give (2S,4R)—N-(2-((5-bromopentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30 mg, 60% yield) as a white solid.

LC-MS (ESI): mass calcd, for C₃₁H₄₂BrFN₄O₅S, 680,7; m/z found, 681,7 [M+H]⁺.

Step B: tert-butyl(2-((5-(2-(((2S,4R)-1-((S)-2-(1-fluorocyclopropane-1-carboxamido) ~3,3-dimethylbutanoyl)-4-hydroxypyrrolidine-2-carboxamido)methyl)-5-(4-methylthiazol-5-yl)phenoxy)pentyl)(methyl)amino)ethyl)carbamate

To a solution of (2S,4R)—N-(2-((5-bromopentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30 mg, 0.05 mmol, 1.0 eq.) and tert-butyl(2-(methylamino)ethyl)carbamate (35 mg, 0.1 mmol, 2.0 eq.) in EtOH (2 mL) was added K₂CO₃ (15 mg, 0.1 mmol, 2.0 eq.). The reaction mixture was stirred overnight at 80° C. The mixture was poured into water and extracted with EtOAc (20 mL×3). The combined organic layers were washed with brine, dried over anhydrous Na₂SO₄ and concentrated under reduced pressure. The residue was purified by flash chromatographer (eluent, 0~10% MeOH in DCM) to give tert-butyl(2-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentyl)(methyl)amino)ethyl)carbamate (30 mg, 73% yield) as a white solid.

LC-MS (ESI): mass calcd. for: C₃₉H₅₉FN₆O₇S, 774.41; m/z found, 775.3 [M+H]⁺.

Step C: (2S,4R)—N-(2-((5-((2-aminoethyl)(methyl)amino)pentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of tert-butyl(2-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentyl)(methyl)amino)ethyl)carbamate (30 mg, 0.04 mmol, 1.0 eq.) in Dioxane (1 mL) was added HCl in Dioxane (1 mL, 4 mol/L) at 0° C. The reaction mixture was stirred for 1 hour at room temperature under N₂ atmosphere. The mixture was concentrated by vacuum to give (2S,4R)—N-(2-((5-((2-aminoethyl)(methyl)amino)pentyl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (30 mg, 80% yield) as a white solid.

LC-MS (ESI): mass calcd, for C₃₄H₃₄FN₆O₅S, 674.3; m/z found, 675.3 [M+H]⁺.

### Step D

Example A35 was synthesized following the same procedures as described in Example A1 (2.0 mg, 7.3% yield). LC-MS (ESI): mass calcd. For C₆₁H₇₁FN₁₂O₈S, 1150.5; m/z found, 1151.4 [M+H]⁺.

### Example A36. 4-amino-N—((S)-6-(6-(4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl) piperazine-1-carbonyl)pyridin-3-yl)-2,3-dihydrobenzo furan-3-yl)-N,1-dimethyl-1H-pyrazolo[4,3-c]quinoline-8-carboxamide

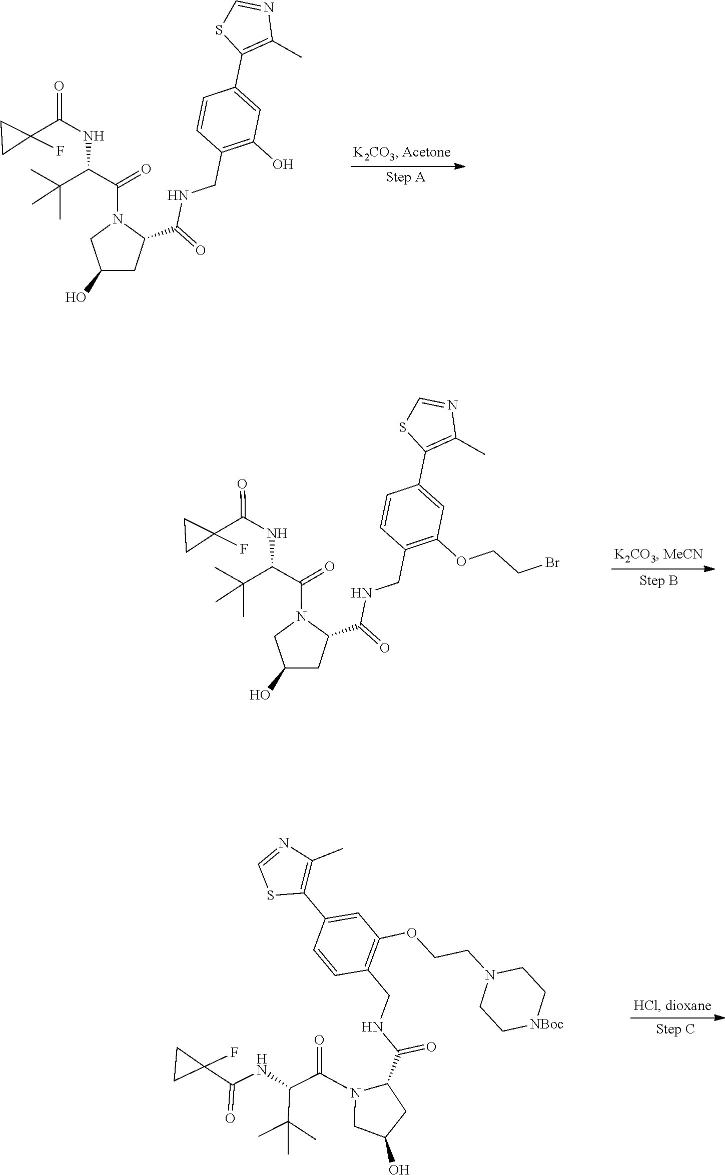 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCBr)c1 ; Cc1ncsc1-c1ccc(CNC(=O)[C@@H]2C[C@@H](O)CN2C(=O)[C@@H](NC(=O)C2(F)CC2)C(C)(C)C)c(OCCN2CCN(C(=O)OC(C)(C)C)CC2)c1]

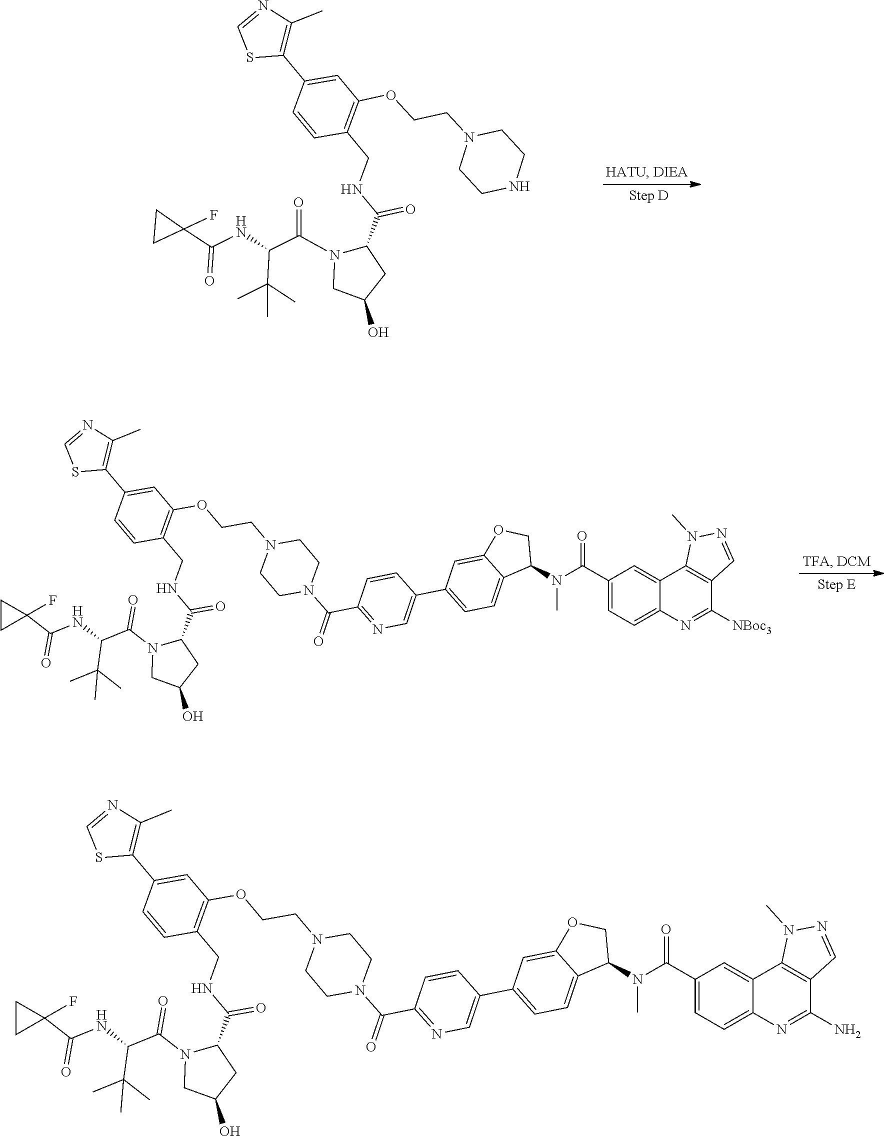

Step A: (2S,4R)—N-(2-(2-bromoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (100 mg, 0.19 mmol, 1.0 eq.), 1,2-dibromoethane (1 ml) in Acetone (8 mL) was added K₂CO₃ (52 mg, 0.38 mmol, 2.0 eq.) at room temperature. The mixture was stirred at 80° C. for 16 hours under N₂ atmosphere. The cooled mixture was filtrated. The filtrate was concentrated under reduced pressure. The residue was purified by flash chromatographer (eluent, 0~5% MeOH in DCM) to give (2S,4R)—N-(2-(2-bromoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (80 mg, 66% yield) as a white solid.

LC-MS (ESI): mass calcd, for C₂₈H₃₆BrFN₄O₅S, 638,6; m/z found, 639,6 [M+H]⁺.

Step B: tert-butyl 4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl) piperazine-1-carboxylate

To a solution of (2S,4R)—N-(2-(2-bromoethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (40 mg, 0.06 mmol, 1.0 eq.), tert-butyl piperazine-1-carboxylate (24 mg, 0.12 mmol, 2.0 eq.) in MeCN (6 mL) was added K₂CO₃ (17 mg, 0.12 mmol, 2.0 eq.). The reaction mixture was stirred overnight at 80° C. The mixture was poured into water at 0° C. and extracted with EtOAc (20 mL×3). The combined organic layers were washed with brine, dried over anhydrous Na₂SO₄ and concentrated under reduced pressure. The residue was purified by flash chromatographer (eluent, 0~10% MeOH in DCM) to give tert-butyl 4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl) piperazine-1-carboxylate (20 mg, 43% yield) as a white solid.

LC-MS (ESI): mass calcd, for C₃₇H₅₃FN₆O₇S, 744.37; m/z found, 745.3 [M+H]⁺.

Step C: (2S,4R)-1-((S)-2-(1-fluorocyclopropane-1-carboxamido)-3,3-dimethylbutanoyl)-4-hydroxy-N-(4-(4-methylthiazol-5-yl)-2-(2-(piperazin-1-yl)ethoxy)benzyl)pyrrolidine-2-carboxamide

To a solution of tert-butyl 4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl) piperazine-1-carboxylate (20 mg, 0.027 mmol) in Dioxane (2 mL) was added HCl in Dioxane (2 mL, 4 mol/L) at 0° C. The reaction mixture was stirred for 1 hour at room temperature under N₂ atmosphere. The mixture was concentrated by vacuum to give (2S,4R)-1-((S)-2-(1-fluorocyclopropane-1-carboxamido)-3,3-dimethylbutanoyl)-4-hydroxy-N-(4-(4-methylthiazol-5-yl)-2-(2-(piperazin-1-yl)ethoxy)benzyl)pyrrolidine-2-carboxamide (17 mg, 99% yield) as a white solid.

LC-MS (ESI): mass calcd, for C₃₂H₄₅FN₆O₅S, 644.32; m/z found, 645.3 [M+H]⁺.

### Steps D-E

Example A36 was synthesized following the same procedures as described in Example A1 (4 mg, 15% yield over 2 steps) as a white solid. LC-MS (ESI): mass calcd, for C₅₉H₆₅FN₁₂O₈S, 1120.5; m/z found, 1121.3 [M+H]⁺.

### Example A37. 4-amino-N—((S)-6-(6-(9-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)-3,9-diazaspiro[5.5]undecane-3-carbonyl)pyridin-3-yl)-2,3-dihydrobenzofuran-3-yl)-N,1-dimethyl-1H-pyrazolo[4,3-c]quinoline-8-carboxamide

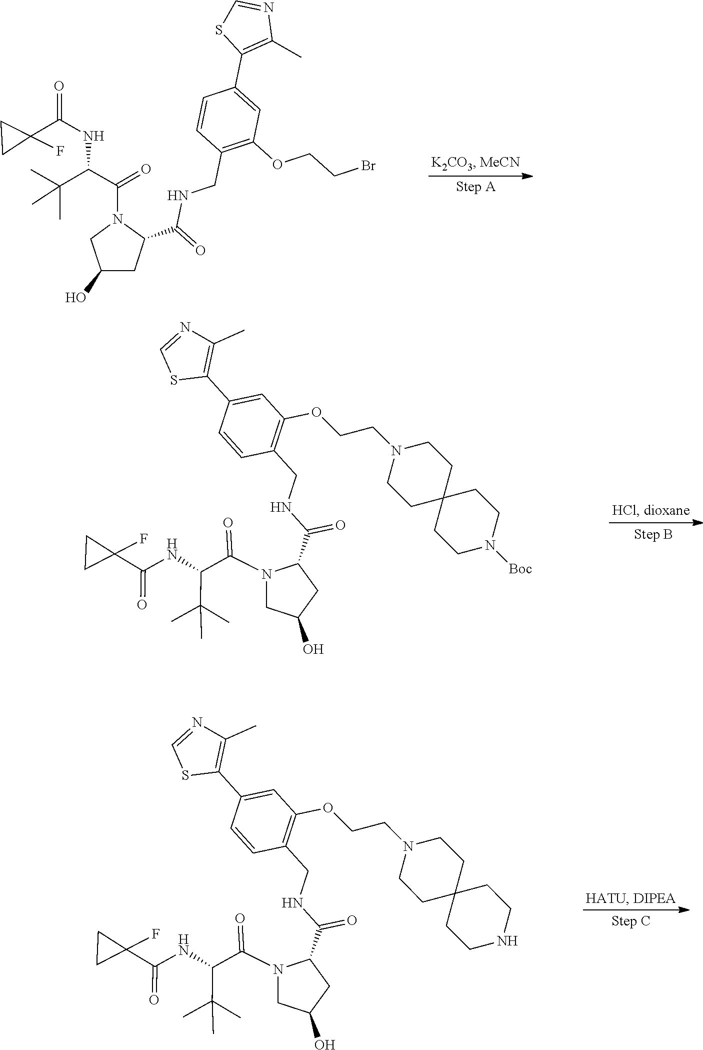 [SMILES: Cc1ncsc1-c1ccc(CNC(=O)[C@@H]2C[C@@H](O)CN2C(=O)[C@@H](NC(=O)C2(F)CC2)C(C)(C)C)c(OCCBr)c1 ; Cc1ncsc1-c1ccc(CNC(=O)[C@@H]2C[C@@H](O)CN2C(=O)[C@@H](NC(=O)C2(F)CC2)C(C)(C)C)c(OCCN2CCC3(CC2)CCN(C(=O)OC(C)(C)C)CC3)c1 ; Cc1ncsc1-c1ccc(CNC(=O)[C@@H]2C[C@@H](O)CN2C(=O)[C@@H](NC(=O)C2(F)CC2)C(C)(C)C)c(OCCN2CCC3(CCNCC3)CC2)c1]

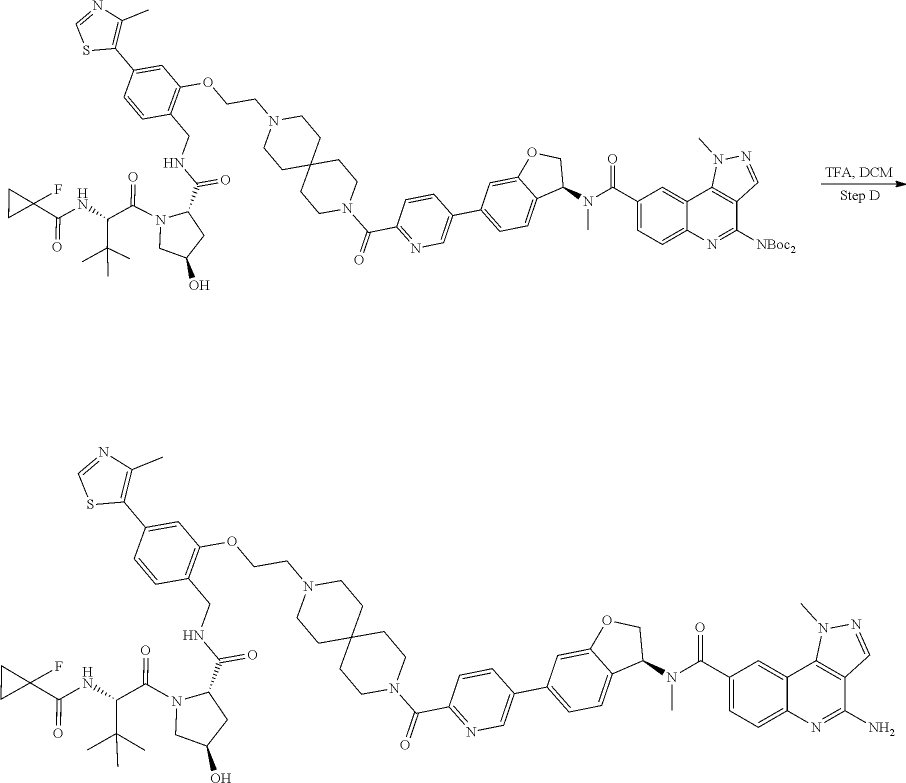

Example A37 was synthesized following the same procedures as described in Example A1 (6 mg, 5% yield over 4 steps) as a white solid. LC-MS (ESI): mass calcd, for C₆₄H₇₃FN₁₂O₈S, 1188.5; m/z found, 1189.6 [M+H]⁺.

### Example A38. 4-amino-N—((S)-6-(6-((2-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)pentyl)(methyl)amino)ethyl)carbamoyl)pyridin-3-yl)-2,3-dihydrobenzofuran-3-yl)-N-methyl-1,3-dihydrofuro[3,4-c][1,7]naphthyridine-8-carboxamide

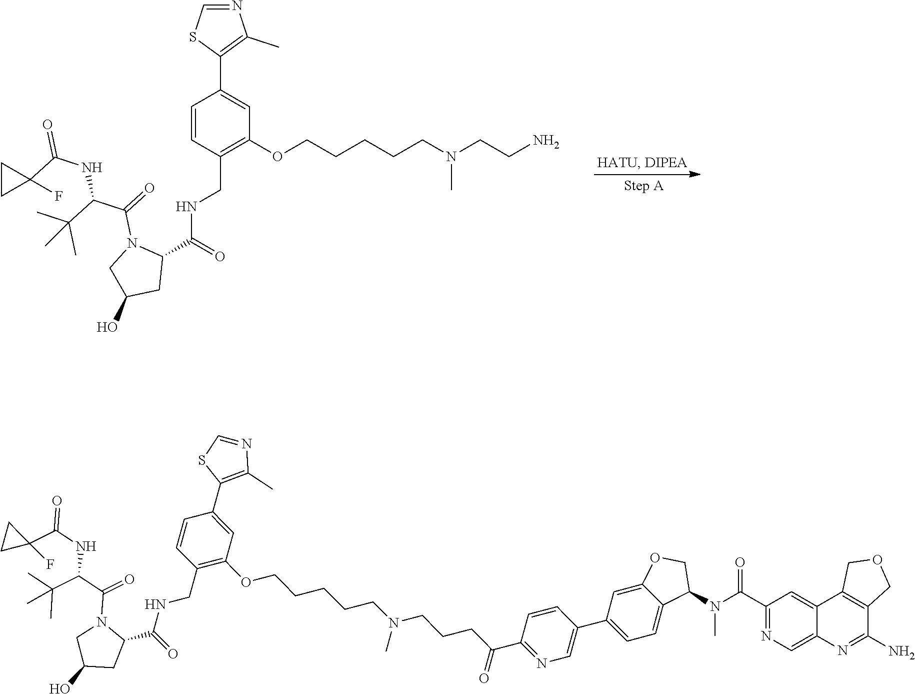 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCN(C)CCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCN(C)CCCC(=O)c2ccc(-c3ccc4c(c3)OC[C@H]4N(C)C(=O)c3cc4c5c(c(N)nc4cn3)COC5)cn2)c1]

Example A38 was synthesized following the same procedures as described in Example A9 (3.3 mg, 15% yield) as a white solid. LC-MS (ESI): mass calcd, for C₆₀H₇₀FN₁₁O₉S, 1139.51; m/z found, 1140.5 [M+H]⁺.

### Example A39. 4-amino-N—((S)-6-(6-((2-(2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamoyl)pyridin-3-yl)-2,3-dihydrobenzofuran-3-yl)-N-methyl-1,3-dihydrofuro[3,4-c][1,7]naphthyridine-8-carboxamide

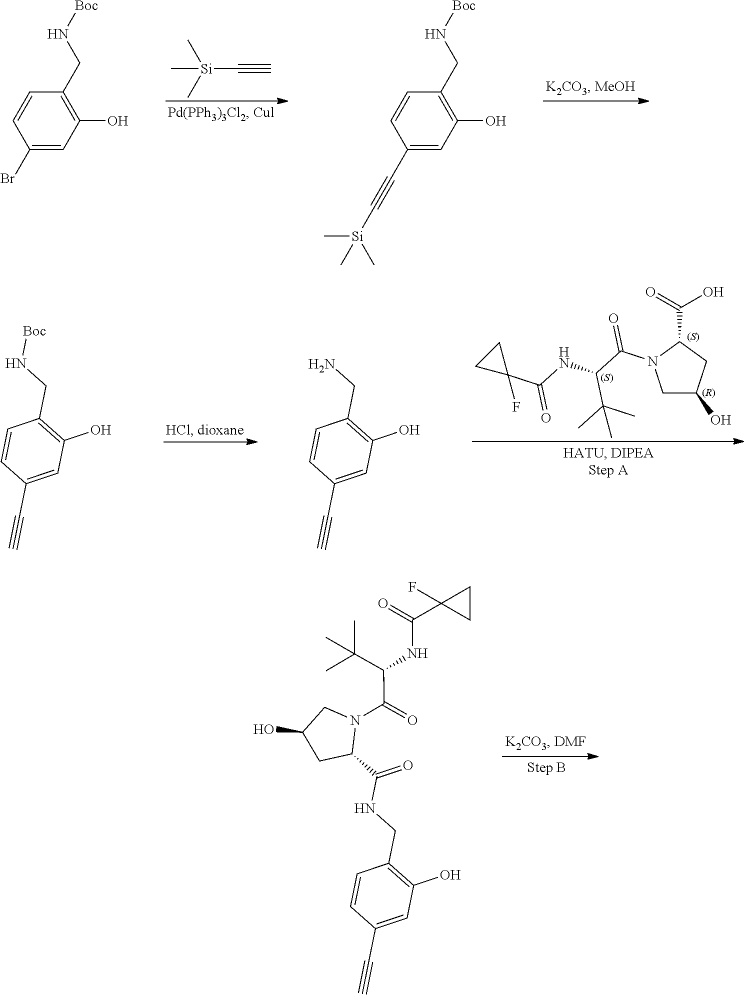

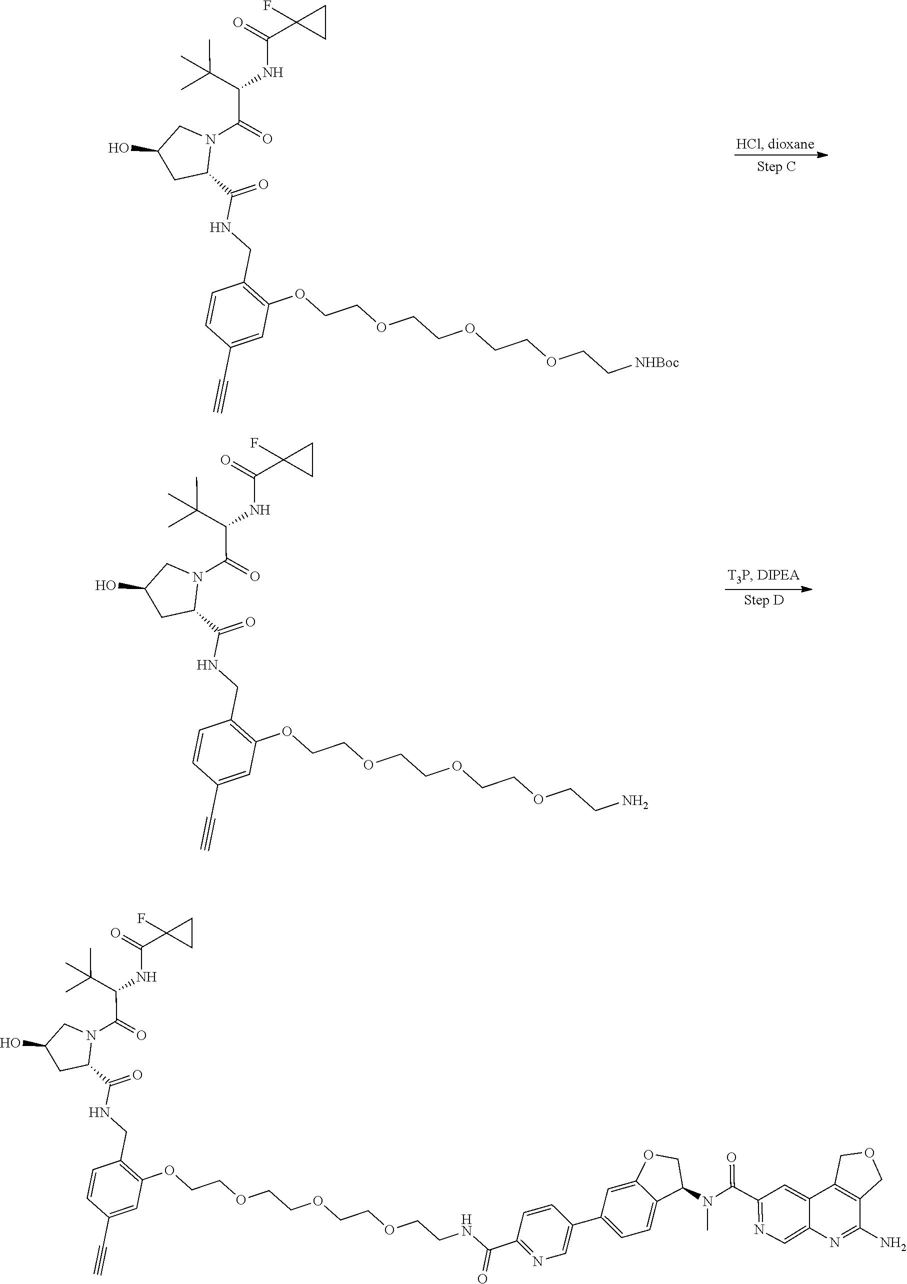 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)OC(C)(C)C)c1 ; C#Cc1ccc(CNC(=O)[C@@H]2C[C@@H](O)CN2C(=O)[C@@H](NC(=O)C2(F)CC2)C(C)(C)C)c(OCCOCCOCCOCCN)c1 ; C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3cc4c5c(c(N)nc4cn3)COC5)cn2)c1]

Step A: (2S,4R)—N-(4-ethynyl-2-hydroxybenzyl)-1-((S)-2-(1-fluorocyclopropane-1-carboxamido)-3, 3-dimethylbutanoyl)-4-hydroxypyrrolidine-2-carboxamide

To a solution of 2-(aminomethyl)-5-ethynylphenol (90 mg, 0.61 mmol, 1.2 eq., WO2023278402) and (2S,4R)-1-((S)-2-(1-fluorocyclopropane-1-carboxamido)-3,3-dimethylbutanoyl)-4-hydroxypyrrolidine-2-carboxylic acid (170 mg, 0.51 mmol, 1.0 eq.) in DMF (15 mL) was added HATU (293 mg, 0.77 mmol, 1.5 eq.) and DIPEA (25 μL, 1.55 mmol, 3.0 eq.). Then the mixture was stirred at room temperature for 1 hour. The resulting solution was diluted with 30 mL of H₂O and extracted with EtOAc (20 mL×3). The combined organic layers were washed with brine, dried over Na₂SO₄ and concentrated under reduced pressure. The residue was purified by prep-HPLC(5~55% MeOH in water) to afford (28,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide (80 mg, 34% yield) as a yellow solid.

LC-MS (ESI): mass calcd, for C₂₄H₃₀FN₃O₅, 459.2; m/z found, 460.3 [M+H]⁺.

Step B: tert-butyl(2-(2-(2-(2-(5-ethynyl-2-(((25, 4R)-1-((S)-2-(1-fluorocyclopropane-1-carboxamido)-3,3-dimethylbutanoyl)-4-hydroxypyrrolidine-2-carboxamido)methyl)phenoxy) ethoxy)ethoxy)ethoxy)ethyl)carbamate

To a solution of (2S,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide (30 mg, 0.06 mmol, 1.0 eq.) and 1-Boc-amino-3,6,9-trioxaundecanyl-11-bromide (35 mg, 0.10 mmol, 1.5 eq.) in DMF (2 mL) was added K₂CO₃ (27 mg, 0.20 mmol, 3.0 eq.), followed by KI (2 mg, 0.01 mmol, 0.2 eq.). Then the mixture was stirred at 75° C. for 3 hours. The resulting solution was diluted with 30 mL of H₂O and extracted with EtOAc (20 mL×3). The combined organic layers were washed with brine, dried over Na₂SO₄ and concentrated under reduced pressure. The residue was purified on silica gel eluting (0~14% MeOH in DCM) to afford tert-butyl(2-(2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamate (50 mg, 78% yield) as a yellow solid.

LC-MS (ESI): mass calcd, for C₃₇H₅₅FN₃₄O₁₀, 734.4; m/z found, 735.3 [M+H]⁺.

Step C: (2S,4R)—N-(2-(2-(2-(2-(2-aminoethoxy)ethoxy)ethoxy)ethoxy)-4-ethynylbenzyl)-1-((S)-2-(1-fluorocyclopropane-1-carboxamido)-3,3-dimethylbutanoyl)-4-hydroxypyrrolidi-ne-2-carboxamide

To solution of tert-butyl(2-(2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamate (50 mg, 0.07 mmol, 1.0 eq.) in MeCN (3 mL) was added HCl-dioxane (1 mL, 4 M/L in dioxane). Then the mixture was stirred at room temperature for 1 hour. The mixture was concentrated in vacuum to afford (2S,4R)—N-(2~(2-(2-(2-(2-aminoethoxy)ethoxy)ethoxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide (40 mg, 93% yield) as a yellow solid.

LC-MS (ESI): mass calcd, for C₃₂H₄₇FN₄O₈, 634.3; m/z found, 635.2 [M+H]⁺.

Step D: 4-amino-N—(S)-6-(6-(2-(2-(2-(2-(5-ethynyl-2-(((28,4R)-1-(S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamoyl)pyridin-3-yl)-2,3-dihydrobenzofuran-3-yl)-N-methyl-1,3-dihydrofuro[3,4-c][1,7]naphthyridine-8-carboxamide

To a solution of (2S,4R)—N-(2-(2-(2-(2-(2-aminoethoxy)ethoxy)ethoxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide (40 mg, 0.06 mmol, 2.0 eq.) and(S)-5-(3-(4-amino-N-methyl-1,3-dihydrofuro[3,4-c][1,7]naphthyridine-8-carboxamido)-2,3-dihydrobenzofuran-6-yl) picolinic acid (15 mg, 0.03 mmol, 1.0 eq.) in DMF (2 mL) was added T3P (20 mg, 0.06 mmol, 2.0 eq.), followed by TEA (17 μL, 0.09 mmol, 3.0 eq.). Then the mixture was stirred at room temperature for 1 hour. The mixture was purified by prep-HPLC(C18, 20~95%, MeCN in H₂O with 0.1% HCOOH) to afford 4-amino-N—((S)-6-(6-((2-(2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamoyl)pyridin-3-yl)-2,3-dihydrobenzofuran-3-yl)-N-methyl-1,3-dihydrofuro[3,4-c][1,7]naphthyridine-8-carboxamide (1.6 mg, 4.7% yield) as a yellow solid.

LC-MS (ESI): mass calcd, for C₅₈H₆₆FN₉O₁₂, 1099.5; m/z found, 1100.3 [M+H]⁺.

### Example A40. 2-amino-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N,3-dimethylquinoli-ne-6-carboxamide

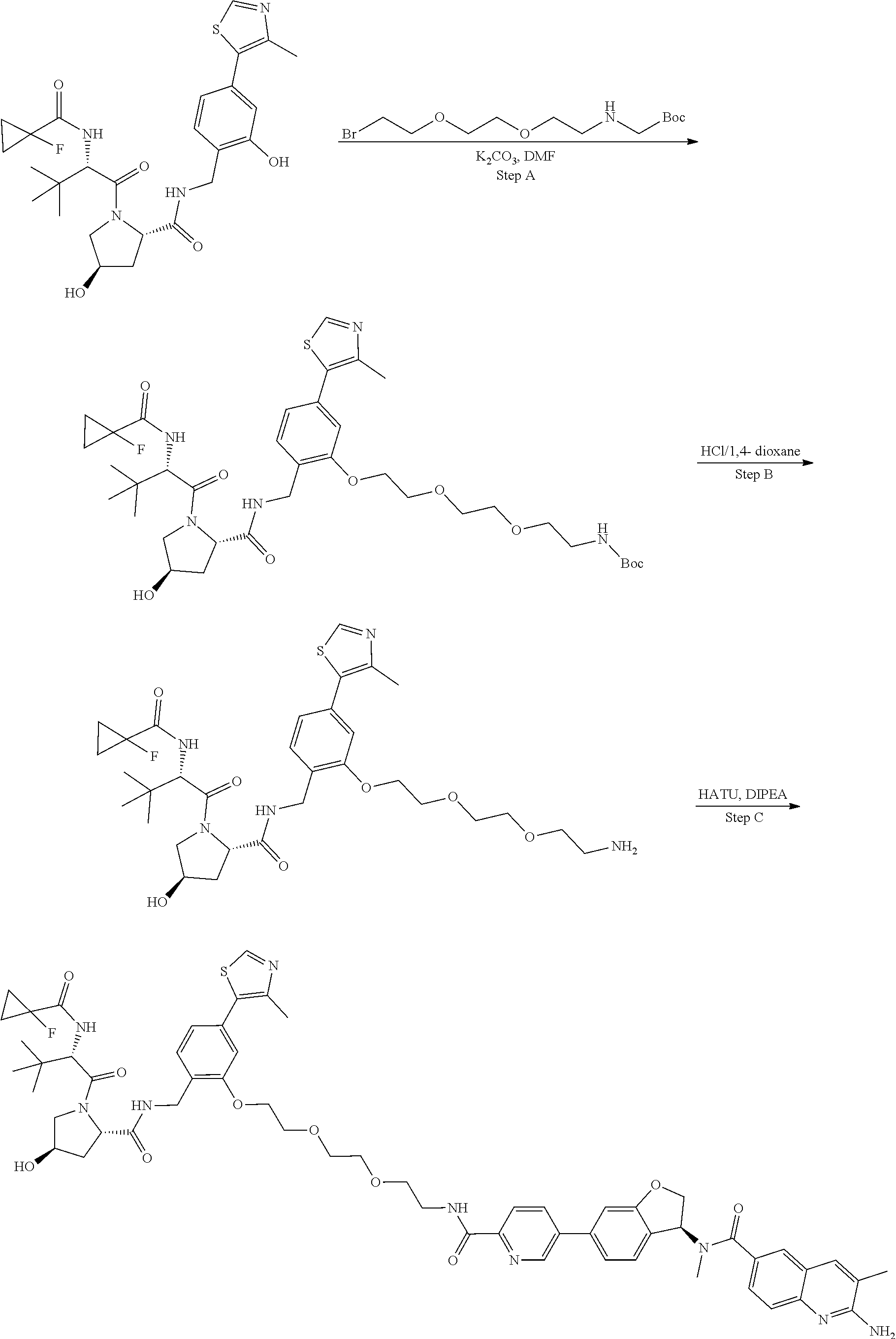 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)CNCCOCCOCCBr ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)OC(C)(C)C)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCN)c1 ; Cc1cc2cc(C(=O)N(C)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N]

Example A40 was synthesized following the same procedures as described in Example A9 (6 mg, 18% yield over 3 steps) as a white solid. LC-MS (ESI): mass calcd, for C₅₈H₆₆FN₉O₁₀S, 1099.46; m/z found, 1100.4 [M+H]. 1H NMR (400 MHz, DMSO-d₆) δ 8.94 (s, 1H), 8.90 (s, 1H), 8.71 (t, J=5.8 Hz, 1H), 8.48 (t, J=5.9 Hz, 1H), 8.25 (dd, J=8.2, 2.1 Hz, 1H), 8.08 (d, J=8.1 Hz, 1H), 7.94 (s, 1H), 7.85 (s, 1H), 7.56 (dd, J=33.1, 11.7 Hz, 3H), 7.40 (d, J=7.8 Hz, 1H), 7.36 (d, J=8.0 Hz, 1H), 7.30 (d, J=10.5 Hz, 2H), 7.00 (s, 1H), 6.95 (d, J=7.8 Hz, 1H), 5.19 (s, 1H), 4.70-4.67 (m, 1H), 4.59 (d, J=9.2 Hz, 1H), 4.51 (s, 1H), 4.33 (d, J=6.9 Hz, 2H), 4.29 (d, J=6.2 Hz, 1H), 4.23 (d, J=5.7 Hz, 1H), 4.18-4.15 (m, 2H), 3.82-3.78 (m, 4H), 3.65-3.63 (m, 4H), 3.60 (d, J=2.4 Hz, 4H), 2.67 (s, 3H), 2.43 (s, 3H), 2.25 (s, 3H), 2.12-2.05 (m, 1H), 1.91 (ddd, J=13.0, 8.9, 4.4 Hz, 1H), 1.40-1.32 (m, 2H), 1.24-1.20 (m, 2H), 0.94 (s, 9H).

### Example A41. 4-amino-3-bromo-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-N,1-dimethyl-1H-pyrazolo[4,3-c]quinoline-8-carboxamide

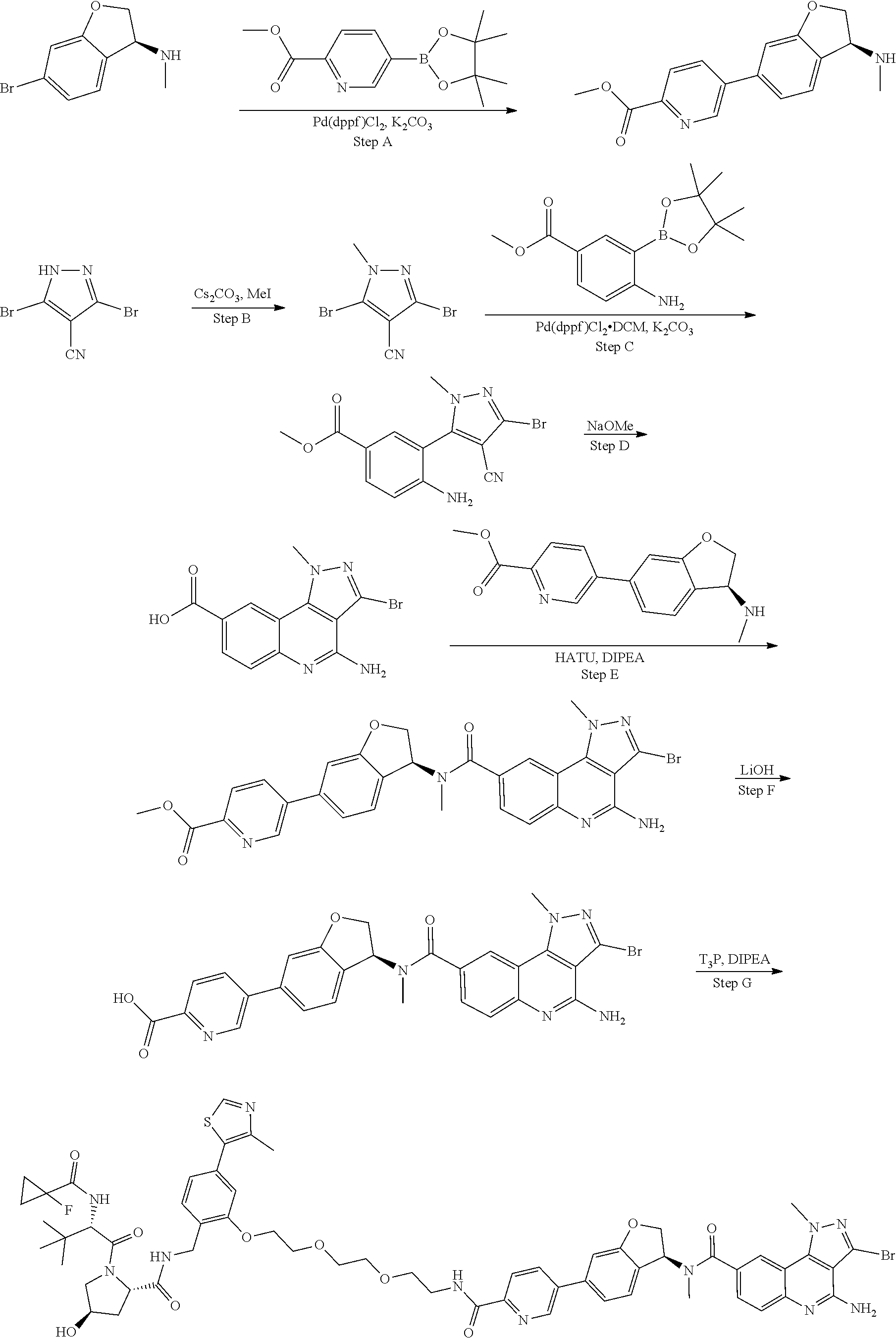

Step A: methyl(S)-5-(3-(methylamino)-2,3-dihydrobenzofuran-6-yl)picolinate

To a mixture of(S)-6-bromo-N-methyl-2,3-dihydrobenzofuran-3-amine (100 mg, 438 μmol, 1.0 eq.), methyl 5-(4,4,5,5-tetramethyl-1,3,2-dioxaborolan-2-yl)picolinate (115 mg, 438 μmol, 1.0 eq.) and K₂CO₃ (182 mg, 1.32 mmol, 3.0 eq.) in 1,4-dioxane (2.0 mL) and H₂O (0.1 mL) was added Pd(dppf)Cl₂ (32 mg, 43.8 μmol, 0.1 eq.) under nitrogen atmosphere. Then the mixture was stirred at 90° C. for 3 hours. The mixture was poured into water and extracted with EtOAc (20 mL×3). The combined organic layers were washed with brine, dried over anhydrous Na₂SO₄ and concentrated under reduced pressure. The residue was purified by flash chromatographer (eluent, 0~3% MeOH in DCM) to give the methyl(S)-5-(3-(methylamino)-2,3-dihydrobenzofuran-6-yl)picolinate (60 mg, 48% yield) as a black oil.

LC-MS (ESI): mass calcd, for C₁₆H₁₆N₂O₃, 284; m/z found, 285.3 [M+H]⁺.

Step B: 3,5-dibromo-1-methyl-1H-pyrazole-4-carbonitrile

To a mixture of 3,5-dibromo-1H-pyrazole-4-carbonitrile (2.0 g, 7.97 mmol, 1.0 eq.) and Cs₂CO₃ (3.9 g, 12.0 mmol, 1.5 eq.) in DMF (20 mL) was added Mel (1.7 g, 748 μL, 12.0 mmol, 1.5 eq.) at room temperature and stirred for 16 hours. The mixture was poured into ice-water and filter. The filter cake was concentrated under reduced pressure to dryness to give the 3,5-dibromo-1-methyl-1H-pyrazole-4-carbonitrile (1.9 g, 90% yield) as a white solid.

LC-MS (ESI): mass calcd, for C₅H₃Br₂N₃, 264.8; m/z found, 266.0 [M+H]⁺. ¹H NMR (400 MHz, DMSO-6) δ 3.32 (s, 3H).

Step C: methyl 4-amino-3-(3-bromo-4-cyano-1-methyl-1H-pyrazol-5-yl)benzoate

To a mixture of methyl 4-amino-3-(4,4,5,5-tetramethyl-1,3,2-dioxaborolan-2-yl)benzoate (209 mg, 755 μmol, 1.0 eq.), 3,5-dibromo-1-methyl-1H-pyrazole-4-carbonitrile (200 mg, 755 μmol, 1.0 eq.) and K₂CO₃ (313 mg, 2.3 mmol, 3.0 eq.) in 1,4-dioxane (4.0 mL) and H₂O (1.0 mL) was added Pd(dppf)Cl₂·DCM (123 mg, 151 μmol, 0.2 eq.) under nitrogen atmosphere. Then was stirred at 50° C. for 5 hours. The mixture was poured into water and extracted with EtOAc (20 mL×3). The combined organic layers were washed with brine, dried over anhydrous Na₂SO₄ and concentrated under reduced pressure. The residue was purified by flash chromatographer (eluent, 0~25% EtOAc in PE) to give the methyl 4-amino-3-(3-bromo-4-cyano-1-methyl-1H-pyrazol-5-yl)benzoate (20 mg, 8% yield) as a white solid.

LC-MS (ESI): mass calcd, for C₁₃H₁₁BrN₄O₂, 334/336; m/z found, 335.1/337.1 [M+H]⁺. ¹H NMR (400 MHz, DMSO-d₆) δ 7.81 (dd, J=8.7, 2.1 Hz, 1H), 7.67 (d, J=2.1 Hz, 1H), 6.83 (d, J=8.7 Hz, 1H), 6.30 (s, 2H), 3.76 (s, 3H), 3.64 (s, 3H).

Step D: 4-amino-3-bromo-1-methyl-1H-pyrazolo[4,3-c]quinoline-8-carboxylic acid

To a solution of methyl 4-amino-3-(3-bromo-4-cyano-1-methyl-1H-pyrazol-5-yl)benzoate (20.0 mg, 59.7 μmol, 1.0 eq.) in MeOH (1 mL) was added Sodium methoxide (934 mg, 1.0 mL, 33% Wt, 5.71 mmol, 95.0 eq.) under nitrogen atmosphere. Then the mixture was stirred at 70° C. for 5 hours. The cooled mixture was concentrated under reduced pressure to dryness to give the 4-amino-3-bromo-1-methyl-1H-pyrazolo[4,3-c]quinoline-8-carboxylic acid (17.0 mg, 89% yield) as a white solid Which was used into next step without further purification.

LC-MS (ESI): mass calcd, for C₁₂H₉BrN₄O₂, 320/322; m/z found, 321.1/323.1 [M+H]⁺.

Step E: methyl(S)-5-(3-(4-amino-3-bromo-N, 1-dimethyl-1H-pyrazolo[4,3-c]quinoline-8-carboxamido)-2,3-dihydrobenzofuran-6-yl)picolinate

To a mixture of 4-amino-3-bromo-1-methyl-1H-pyrazolo[4,3-c]quinoline-8-carboxylic acid (15 mg, 46.7 μmol, 1.0 eq.), methyl(S)-5-(3-(methylamino)-2,3-dihydrobenzofuran-6-yl)picolinate (13 mg, 46.7 μmol, 1.0 eq.) and DIEA (18 mg, 24 μL, 140 μmol, 3.0 eq.) in DMF (1 mL) was added T3P (22.3 mg, 20.6 μL, 70.1 μmol, 1.5 eq., 50% in DMF) at room temperature. Then was stirred for 1 hour. The mixture was filtered and purified by flash chromatographer (eluent, 0~3% MeOH in DCM) to give the methyl(S)-5-(3-(4-amino-3-bromo-N,1-dimethyl-1H-pyrazolo[4,3-c]quinoline-8-carboxamido)-2,3-dihydrobenzofuran-6-yl)picolinate (15 mg, 55% yield) as a white solid.

LC-MS (ESI): mass calcd, for C₂₈H₂₃BrN₆O₄, 586/588; m/z found, 587.3/589.3 [M+H]⁺.

Step F: (S)-5-(3-(4-amino-3-bromo-N,1-dimethyl-1H-pyrazolo[4,3-c]quinoline-8-carboxamido)-2,3-dihydrobenzofuran-6-yl)picolinic acid

To a mixture of methyl(S)-5-(3-(4-amino-3-bromo-N,1-dimethyl-1H-pyrazolo[4,3-c]quinoline-8-carboxamido)-2,3-dihydrobenzofuran-6-yl)picolinate (15.0 mg, 25.5 μmol, 1.0 eq.) in THF (2.0 mL) was added H₂O (0.4 mL) and LiOH·H2O (1.83 mg, 76.6 μmol, 3.0 eq.) at room temperature. Then the mixture was stirred at room temperature for 2 hours. The mixture was concentrated under reduced pressure to dryness to give the(S)-5-(3-(4-amino-3-bromo-N,1-dimethyl-1H-pyrazolo[4,3-c]quinoline-8-carboxamido)-2,3-dihydrobenzofuran-6-yl) picolinic acid (13 mg, 88% yield) as a white solid.

LC-MS (ESI): mass calcd, for C₂₇H₂₁BrN₆O₄, 337/339; m/z found, 338/340 [M+H]⁺.

Step G: 4-amino-3-bromo-N—(S)-6-(6-(2-(2-(2-(2-(((28,4R)-1-(S)-2-(1-fluorocyclopropane-I-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)-carbamoyl) pyridin-3-yl)-2,3-dihydrobenzofuran-3-yl)-N, 1-dimethyl-1H-pyrazolo[4,3-c]quinoline-8-carboxamide

To a mixture of (2S,4R)—N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (15.0 mg, 22.7 μmol, 1.0 eq.), (S)-5-(3-(4-amino-3-bromo-N, 1-dimethyl-1H-pyrazolo[4,3-c]quinoline-8-carboxamido)-2,3-dihydrobenzofuran-6-yl)picolinic acid (13.0 mg, 22.7 μmol, 1.0 eq.) and DIEA (8.8 mg, 11.8 μL, 68.0 μmol, 3.0 eq.) in DMF (1.0 mL) was added T3P (10.8 mg, 10.0 μL, 34.0 μmol, 1.5 eq., 50% in EtOAc) at room temperature. Then the mixture was stirred at room temperature for 1 hour. The mixture was filtered and purified by prep-HPLC(C18, 10~95%, MeCN in H₂O with 0.1% FA) to give the 4-amino-3-bromo-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N,1-dimethyl-1H-pyrazolo[4,3-c]quinoline-8-carboxamide (3 mg, 11% yield) as a white solid.

LC-MS (ESI): mass calcd, for C₅₉H₆₅BrFN₁₁O₁₀S, 1217/1219; m/z found, 1217.6/1219.6 [M+H]⁺.

### Example A42. 2-amino-N—((S)-6-(6-(((1S,4S)-4-(2-(2-(((2,4R)-1-((S)-2-(1-fluorocyclopropane-1-carboxamido)-3,3-dimethylbutanoyl)-4-hydroxypyrrolidilidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,3-dimethyl-quinoline-6-carboxamide

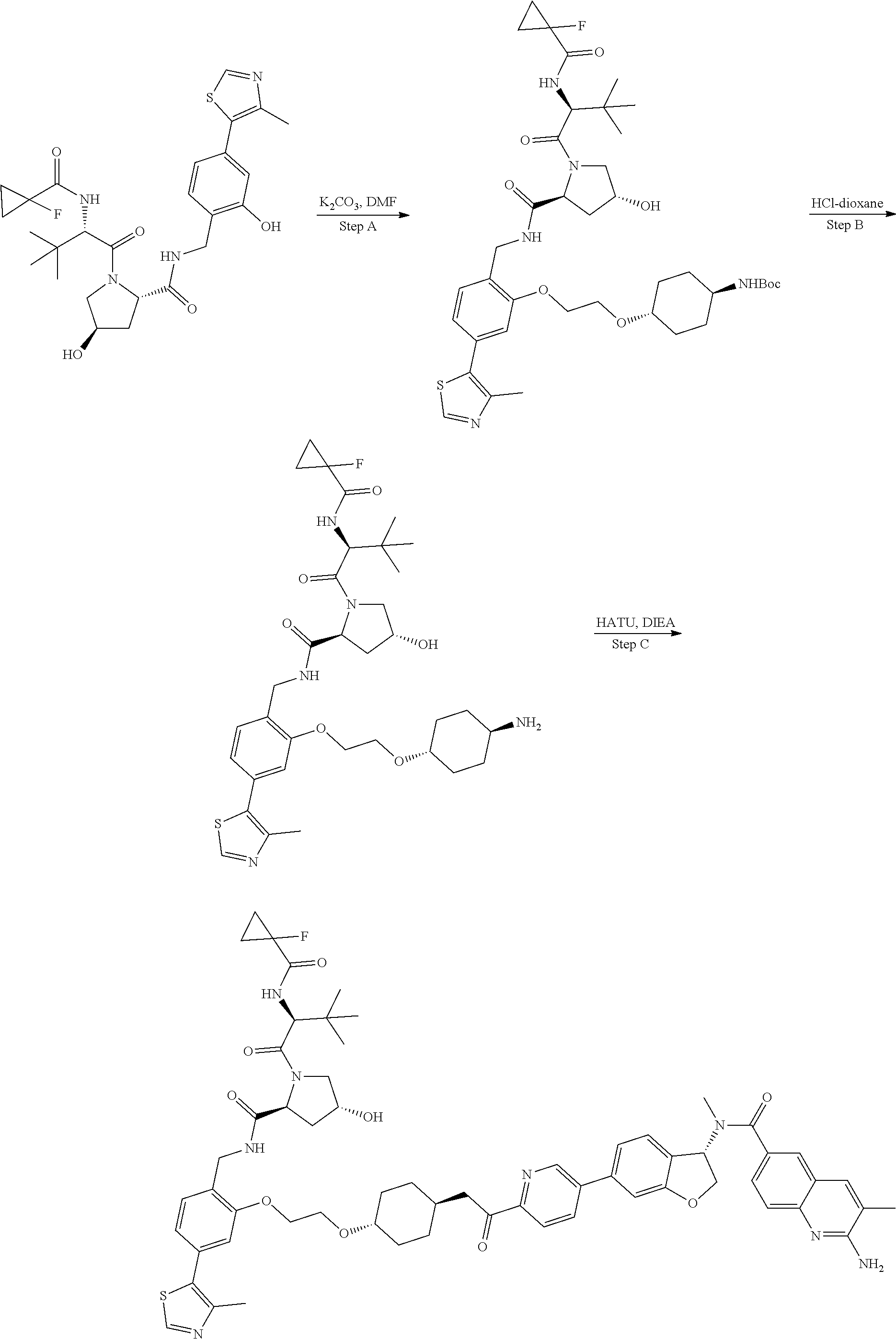 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)OC(C)(C)C)CC2)c1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1 ; Cc1cc2cc(C(=O)N(C)[C@@H]3COc4cc(-c5ccc(C(=O)C[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N]

Example A42 was synthesized following the same procedures as described in Example A21 (6.5 mg, 11% yield over 3 steps) as a white solid. LC-MS (ESI): mass calcd. for C₆₀H₆₈FN₉O₉S, 1109.48; m/z found, 1110.4 [M+H], ¹HNMR (400 MHz, DMSO-d₆) δ 8.99 (s, 1H), 8.92 (d, J=1.8 Hz, 1H), 8.51-8.46 (m, 2H), 8.27 (dd, J=8.2, 2.2 Hz, 1H), 8.08 (d, J=8.3 Hz, 1H), 7.80 (d, J=13.7 Hz, 2H), 7.57-7.48 (m, 3H), 7.40 (t, J=9.0 Hz, 2H), 7.33 (s, 1H), 7.29 (dd, J=9.4, 2.6 Hz, 1H), 7.05 (d, J=1.4 Hz, 1H), 6.97 (dd, J=7.7, 1.4 Hz, 1H), 6.50 (s, 2H), 5.18 (d, J=3.6 Hz, 1H), 4.71 (dd, J=18.7, 14.4 Hz, 2H), 4.60 (d, J=9.1 Hz, 1H), 4.52 (t, J=8.2 Hz, 1H), 4.35 (s, 1H), 4.33-4.20 (m, 2H), 4.18 (s, 2H), 3.80 (d, J=4.1 Hz, 3H), 3.64 (dt, J=22.3, 7.4 Hz, 2H), 3.41 (s, 1H), 2.67 (s, 3H), 2.47 (s, 3H), 2.23 (s, 3H), 2.11 (d, J=12.5 Hz, 1H), 2.06 (d, J=14.6 Hz, 2H), 1.97-1.91 (m, 1H), 1.86 (d, J=9.8 Hz, 2H), 1.54-1.46 (m, 2H), 1.35 (dd, J=11.3, 7.3 Hz, 3H), 1.26-1.20 (m, 3H), 0.96 (s, 9H).

### Example A43. 4-amino-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-1,3-dihydrofuro[3,4-c][1,7]naphthyri-dine-8-carboxamide

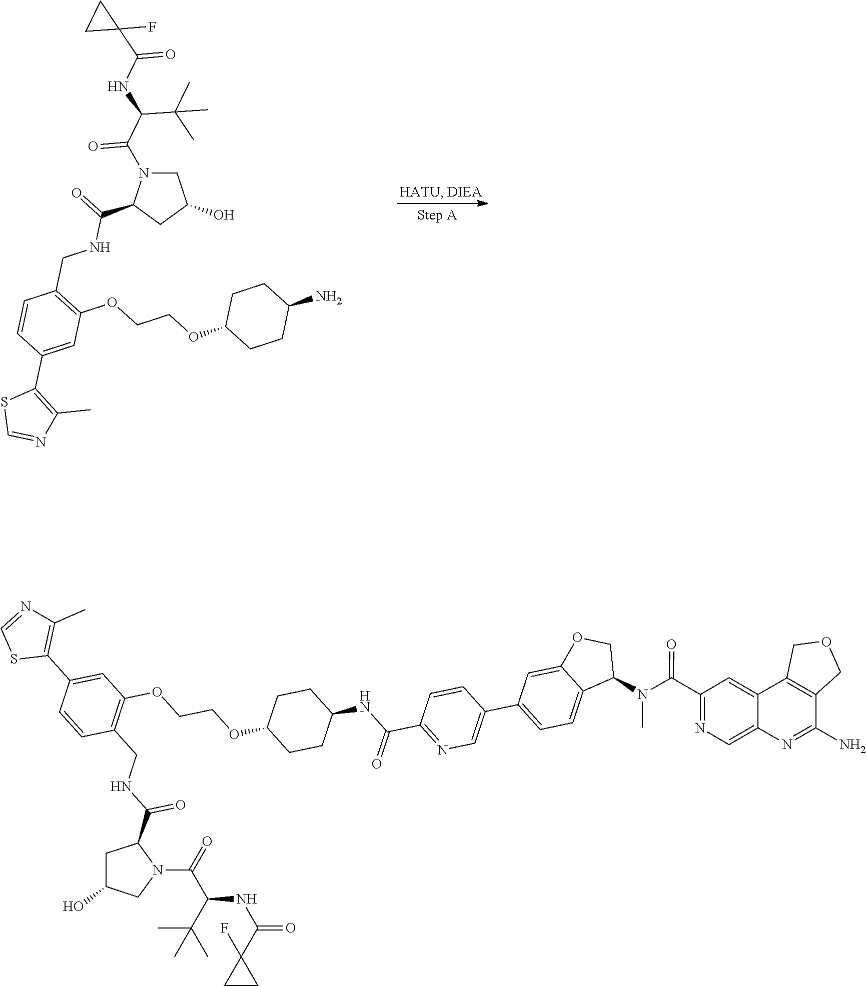 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4cc5c6c(c(N)nc5cn4)COC6)cn3)CC2)c1]

Example A43 was synthesized following the same procedures as described in Example A21 (3.1 mg, 11% yield) as a white solid. LC-MS (ESI): mass calcd, for C₆₀H₆₇FN₁₀O₁₀S, 1139.3; m/z found, 1140.2 [M+H]⁺.

### Example A44. 1′-(4-amino-1-methyl-1H-pyrazolo[4,3-c]quinoline-8-carbonyl)-N-(17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl)-3H-spiro[isobenzofuran-1,3′-pyrrolidine]-5-carboxamide

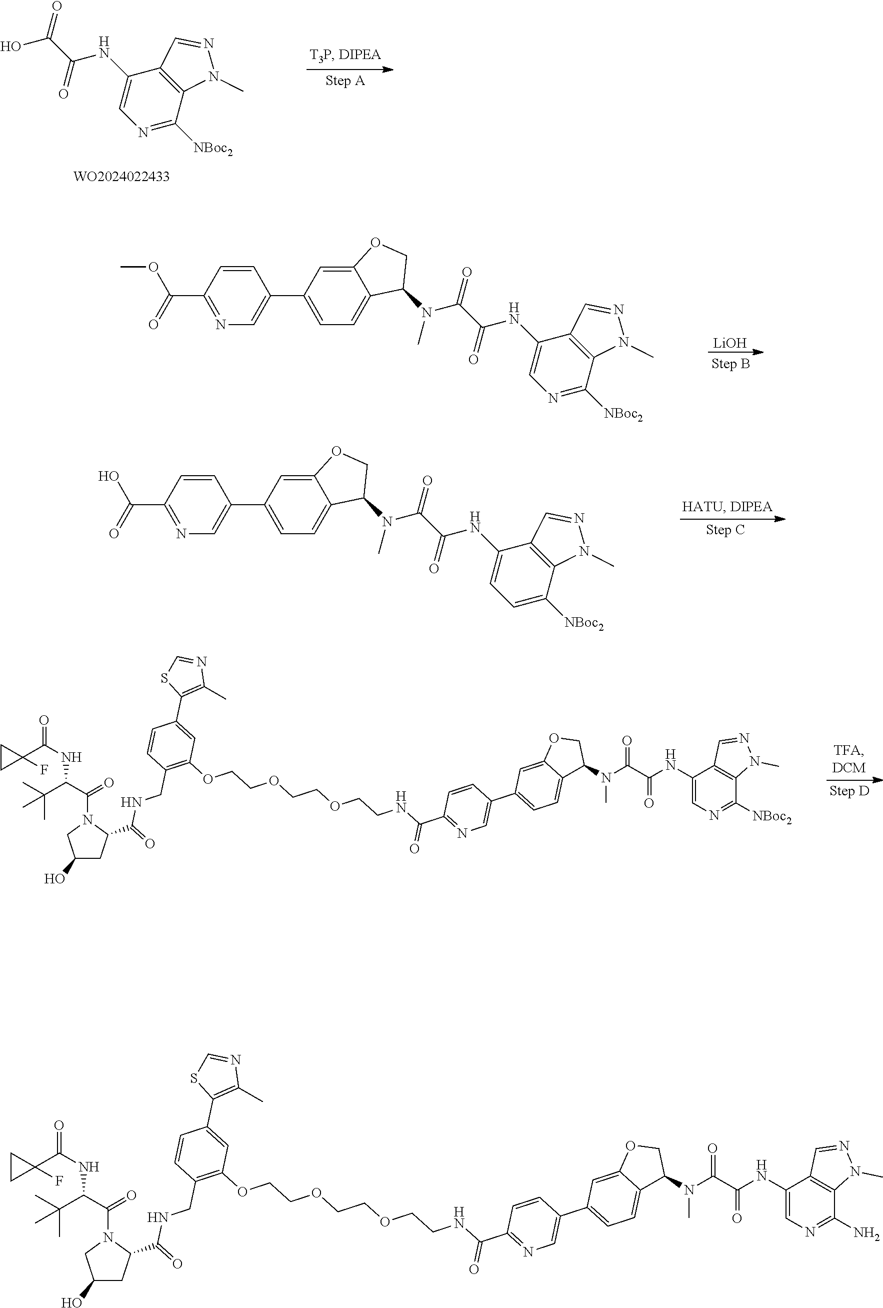

Step A: methyl(S)-5-(3-(2-((7-(bis(tert-butoxycarbonyl)amino)-1-methyl-1H-pyrazolo[3,4-c]pyridin-4-yl)amino)-N-methyl-2-oxoacetamido)-2,3-dihydrobenzofuran-6-yl)picolinate

To a solution of 2-((7-(bis(tert-butoxycarbonyl)amino)-1-methyl-1H-pyrazolo[3,4-c]pyridin-4-yl)amino)-2-oxoacetic acid (35 mg, 80.4 μmol, 1.0 eq. WO2024022433), methyl(S)-5-(3-(methylamino)-2,3-dihydrobenzofuran-6-yl)picolinate (23 mg, 80.4 μmol, 1.0 eq.) and T3P (0.07 mL, 121 μmol, 1.5 eq. 50% in DMF) in DMF (1 mL) was added DIEA (31.2 mg, 42.0 μmol, 3.0 eq.) at room temperature. The reaction mixture was stirred at room temperature for 30 mins under N₂ atmosphere. The mixture was diluted with water (10 mL) and extracted with EtOAc (10 mL×3). The combined organic layers were washed with brine, dried over anhydrous Na₂SO₄ and concentrated under reduced pressure to dryness to give methyl(S)-5-(3-(2-((7-(bis(tert-butoxycarbonyl)amino)-1-methyl-1H-pyrazolo[3,4-c]pyridin-4-yl)amino)-N-methyl-2-oxoacetamido)-2,3-dihydrobenzofuran-6-yl)picolinate (20 mg, 36% yield) as yellow oil.

LC-MS (ESI): mass calcd, for C₃₅H₃₉N₇O₉, 701.28; m/z found, 702.3 [M+H]⁺.

Step B: (S)-5-(3-(2-((7-(bis(tert-butoxycarbonyl)amino)-1-methyl-1H-pyrazolo[3,4-c]pyridin-4-yl)amino)-N-methyl-2-oxoacetamido)-2,3-dihydrobenzofuran-6-yl)picolinic acid

To a mixture of methyl(S)-5-(3-(2-((7-(bis(tert-butoxycarbonyl)amino)-1-methyl-1H-pyrazolo[3,4-c]pyridin-4-yl)amino)-N-methyl-2-oxoacetamido)-2,3-dihydrobenzofuran-6-yl)picolinate (20 mg, 28.5 μmol, 1.0 eq.) in THF (2.0 mL) was added H₂O (0.4 mL) and LiOH·H₂O (2.1 mg, 85.5 μmol, 3.0 eq.) at room temperature. Then the mixture was stirred at room temperature for 2 hours. The mixture was concentrated under reduced pressure to dryness to give the(S)-5-(3-(4-amino-N-methyl-1,3-dihydrofuro[3,4-c][1,7]naphthyridine-8-carboxamido)-2,3-dihydrobenzo furan-6-yl)picolinic acid (18 mg, 92% yield) as a white solid.

LC-MS (ESI): mass calcd, for C₃₄H₃₇N₇O₉, 687.27; m/z found, 688.3 [M+H]⁺.

Step C: tert-butyl (tert-butoxycarbonyl)(4-(2-(S)-6-16-(2-(2-(2-(2-(((2S,4R)-1-(S)-2-(1-fluorocyclopropane-1-carboxamido)-3,3-dimethylbutanoyl)-4-hydroxypyrrolidi-ne-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)(methyl)amino)-2-oxoacetamido)-1-methyl-1H-pyrazolo[3,4-c]pyridin-7-yl)carbamate

To a solution of (2S,4R)—N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (15 mg, 22.6 μmol, 1.0 eq.), (S)-5-(3-(2-((7-(bis(tert-butoxycarbonyl)amino)-1-methyl-1H-pyrazolo[3,4-c]pyridin-4-yl)amino)-N-methyl-2-oxoacetamido)-2,3-dihydrobenzofuran-6-yl)picolinic acid (15.5 mg, 22.6 μmol, 1.0 eq.) and T3P (0.09 mL, 33.9 μmol, 1.5 eq. 50% in DMF) in DMF (1 mL) was added DIEA (8.8 mg, 67.8 μmol, 3.0 eq.) at room temperature. The reaction mixture was stirred at room temperature for 30 mins under N₂ atmosphere. The mixture was diluted with water (10 mL) and extracted with EtOAc (10 mL×3). The combined organic layers were washed with brine, dried over anhydrous Na₂SO₄ and concentrated under reduced pressure to dryness to give tert-butyl (tert-butoxycarbonyl)(4-(2-(((S)-6-(6-((2-(2-(2-(2-(((2S,4R)-1-((S)-2-(1-fluorocyclocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)(methyl)amino)-2-oxoacetamido)-1-methyl-1H-pyrazolo[3,4-c]pyridin-7-yl)carbamate (10 mg, 33% yield) as a brown oil.

LC-MS (ESI): mass calcd, for C₆₆H₈₁FN₁₂O₁₅S, 1332.56; m/z found, 1333.6 [M+H]⁺.

Step D: N1-(7-amino-1-methyl-1H-pyrazolo[3,4-c]pyridin-4-yl)-N2-((S)-6-(6-((2-(2-(2-(2-(((2S, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) ethoxy)ethyl)carbamoyl)pyridin-3-yl)-2,3-dihydrobenzofuran-3-yl)-N2-methyloxalamide

To a solution of tert-butyl (tert-butoxycarbonyl)(4-(2-(((S)-6-(6-((2-(2-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin -3-yl)-2,3-dihydrobenzofuran-3-yl)(methyl)amino)-2-oxoacetamido)-1-methyl-1H-pyrazolo[3,4-c]pyridin-7-yl)carbamate (10 mg, 7.5 μmol, 1.0 eq.) in DCM (2.0 mL) was added TFA (0.7 mL) under N₂ atmosphere. The mixture was concentrated under reduced pressure to dryness. The residue was purified by prep-HPLC(C18, 20~95%, MeCN in H₂O with 0.1% HCOOH) to give N1-(7-amino-1-methyl-1H-pyrazolo[3,4-c]pyridin-4-yl)-N2-((S)-6-(6-((2-(2-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-N2-methyloxalamide (2.0 mg, 23% yield) as a white solid.

LC-MS (ESI): mass calcd, for C₅₆H₆₅FN₁₂O₁₁S, 1132.46; m/z found, 1133.9 [M+H]⁺.

### Example A45. 4-amino-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N-isopropyl-1,3-dihydrofuro[3,4-c][1,7]naphthyridine-8-carboxamide

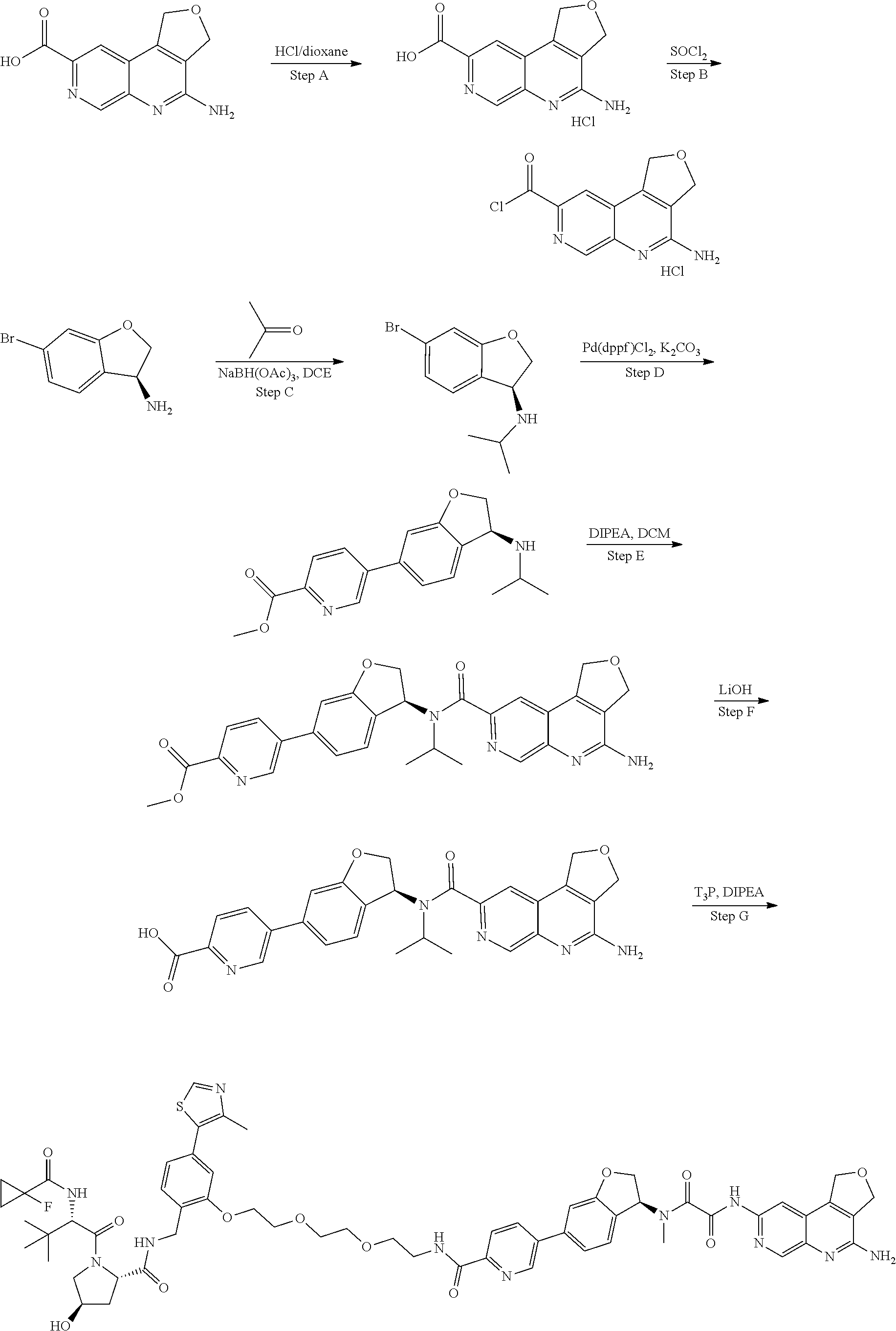

Steps A-B: 4-amino-1,3-dihydrofuro[3,4-c][1,7]naphthyridine-8-carbonyl chloride

To a solution of 4-amino-1,3-dihydrofuro[3,4-c][1,7]naphthyridine-8-carboxylic acid (100 mg, 433 μmol, 1.0 eq.) in DCM (3.0 mL) was added HCl-dioxane (0.1 mL) and stirred at room temperature for 10 min. The resulting solution was concentrated under reduced pressure. The resulting yellow solid was treated with SOCl₂ (3 mL) followed by DMF (0.05 mL). The reaction mixture was stirred at 55° C. for 1 hour. The reaction mixture was cooled to room temperature, diluted with DCM and filtered to give 4-amino-1,3-dihydrofuro[3,4-c][1,7]naphthyridine-8-carbonyl chloride (80 mg, 74% yield) as a yellow solid.

LC-MS (ESI): mass calcd, for C₁₁H₈ClN₃O₂, 249.0; m/z found, 250.0 [M+H]⁺.

Step C: (S)-6-bromo-N-isopropyl-2,3-dihydrobenzofuran-3-amine

To a solution of(S)-6-bromo-2,3-dihydrobenzofuran-3-amine (150 mg, 701 μmol, 1.0 eq.) and AcOH (0.02 mL) in DCE (7.5 mL) was added acetone (1.5 mL). Then mixture was stirred for 2 hours at 20° C. Then Na(OAc)₃BH (297 mg, 1.40 mmol, 2.0 eq.) was added and the mixture was stirred at room temperature for 2 hours. The resulting solution was diluted with 30 mL of water and extracted with EtOAc (20 mL×3). The combined organic layers were washed with brine (50 mL), dried over Na₂SO₄ and concentrated under reduced pressure. The residue was purified by flash column chromatography on silica gel (100-200 mesh silica gel, 0-2% MeOH in DCM) to give(S)-6-bromo-N-isopropyl-2,3-dihydrobenzofuran-3-amine (120 mg, 70% yield) as a brown solid.

LC-MS (ESI): mass calcd, for C₁₁H₁₄BrNO, 255.1; m/z found, 256.2 [M+H]⁺.

Step D: methyl(S)-5-(3-(isopropylamino)-2,3-dihydrobenzofuran-6-yl)picolinate

To a solution of(S)-6-bromo-N-isopropyl-2,3-dihydrobenzofuran-3-amine (120.0 mg, 468 μmol, 1.0 eq.), methyl 5-(4,4,5,5-tetramethyl-1,3,2-dioxaborolan-2-yl)picolinate (160.2 mg, 609 μmol, 1.3 eq.) and K₂CO₃ (194.2 mg, 1.405 mmol, 3.0 eq.) in 1,4-Dioxane (4 mL) and Water (200 μL) was added PdCl₂ (dppf)(34.28 mg, 46.85 μmol, 0.1 eq.). The mixture was stirred at 90° C. for 3 hours. The resulting solution was diluted with 30 mL of water and extracted with EtOAc (20 mL×3). The combined organic layers were washed with brine 50 mL), dried over Na₂SO₄ and concentrated under reduced pressure. The residue was purified by flash column chromatography on silica gel (100-200 mesh silica gel, 0-3% MeOH in DCM) to give methyl(S)-5-(3-(isopropylamino)-2,3-dihydrobenzofuran-6-yl)picolinate (90 mg, 61% yield) as a yellow solid.

LC-MS (ESI): mass calcd, for C₁₈H₂₀N₂O₃, 312.3; m/z found, 313.2 [M+H]⁺.

Step E: methyl(S)-5-(3-(4-amino-N-isopropyl-1,3-dihydrofuro[3,4-c][1,7]naphthyridine-8-carboxamido)-2,3-dihydrobenzofuran-6-yl)picolinate

To a solution of methyl(S)-5-(3-(isopropylamino)-2,3-dihydrobenzofuran-6-yl)picolinate (30.0 mg, 96 μmol, 1.0 eq.), DIEA (42 μL, 240 μmol, 2.5 eq.) in DCM (3.00 mL) was added 4-amino-1,3-dihydrofuro[3,4-c][1,7]naphthyridine-8-carbonyl chloride (25.0 mg, 120 μmol, 1.1 eq.). The mixture was stirred at room temperature for 1.5 hours. The resulting solution was diluted with 10 mL of water and extracted with DCM (10 mL×3). The combined organic layers were washed with brine (20 mL), dried over Na₂SO₄ and concentrated under reduced pressure. The residue was purified by flash column chromatography on silica gel (100-200 mesh silica gel, 0-7% MeOH in DCM) to give methyl(S)-5-(3-(4-amino-N-isopropyl-1,3-dihydrofuro[3,4-c][1,7]naphthyridine-8-carboxamido)-2,3-dihydrobenzofuran-6-yl)picolinate (15.0 mg, 56% yield) as a yellow solid.

LC-MS (ESI): mass calcd, for C₂₉H₂₇N₅O₅, 525.2; m/z found, 526.1 [M+H]⁺.

Step F: (S)-5-(3-(4-amino-N-isopropyl-1,3-dihydrofuro[3,4-c][1,7]naphthyridine-8-carboxamido)-2,3-dihydrobenzofuran-6-yl)picolinic acid

To a solution of methyl(S)-5-(3-(4-amino-N-isopropyl-1,3-dihydrofuro[3,4-c][1,7]naphthyridine-8-carboxamido)-2,3-dihydrobenzofuran-6-yl)picolinate (15.0 mg, 28.5 μmol, 1.0 eq.) in THF (1.0 mL) and Water (1.0 mL) was added Lithium hydroxide monohydrate (2.4 mg, 57.1 μmol, 2.0 eq.) and stirred at room temperature for 50 min. The resulting solution was concentrated under reduced pressure to give(S)-5-(3-(4-amino-N-isopropyl-1,3-dihydrofuro[3,4-c][1,7]naphthyridine-8-carboxamido)-2,3-dihydrobenzofuran-6-yl)picolinic acid (14.0 mg, 96% yield) as a yellow solid.

LC-MS (ESI): mass calcd, for C₂₈H₂₅N₅O₅, 511.2; m/z found, 512.1 [M+H]⁺.

Step G: 4-amino-N—(S)-6-(6-(2-(2-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N-isopropyl-1,3-dihydrofuro[3,4-c][1,7]naphthyridine-8-carboxamide

To a solution of(S)-5-(3-(4-amino-N-isopropyl-1,3-dihydrofuro[3,4-c][1,7]naphthyridine-8-carboxamido)-2,3-dihydrobenzofuran-6-yl)picolinic acid (15 mg, 23.5 μmol, 1.0 eq.), (2S,4R)—N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (31 mg, 46.9 μmol 2.0 eq.) and T3P (27.6 μL, 46.9 μmol, 2.0 eq.) in DMF (3.0 mL) was added TEA (10 μL, 71 μmol, 3.0 eq.). The mixture was stirred at 50° C. for 1 hour. The resulting solution was diluted with 10 mL of water and extracted with EtOAc (10 mL×3). The combined organic layers were washed with brine (20 mL), dried over Na₂SO₄ and concentrated under reduced pressure. The residue was purified by prep-HPLC(C18, 20~95%, MeCN in H₂O with 0.1% HCOOH) to give 4-amino-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N-isopropyl-1,3-dihydrofuro[3,4-c][1,7]naphthyridine-8-carboxamide (4.3 mg, 15% yield) as a yellow solid.

LC-MS (ESI): mass calcd, for C₆₀H₆₉FN₁₀O₁₁S, 1156.4; m/z found, 1158 [M+H]⁺. ¹H NMR (400 MHz, DMSO-d₆) δ 8.97-8.95 (m, 1H), 8.92-8.83 (m, 2H), 8.71 (t, J=5.6 Hz, 1H), 8.49 (t, J=5.9 Hz, 1H), 8.22 (d, J=7.4 Hz, 1H), 8.07 (d, J=7.9 Hz, 1H), 7.62 (s, 1H), 7.40 (d, J=7.7 Hz, 1H), 7.31-7.27 (m, 2H), 7.25-7.16 (m, 2H), 7.02 (s, 3H), 6.95 (d, J=7.6 Hz, 1H), 5.43-5.38 (m, 1H), 5.32 (s, 1H), 5.17 (d, J=3.3 Hz, 1H), 5.03 (s, 2H), 4.81-4.75 (m, 1H), 4.69-4.65 (m, 1H), 4.59 (d, J=9.3 Hz, 1H), 4.53 (d, J=8.3 Hz, 1H), 4.38-4.32 (m, 2H), 4.29 (d, J=6.1 Hz, 1H), 4.23 (d, J=5.3 Hz, 1H), 4.18 (s, 2H), 3.82-3.80 (m, 2H), 3.66-3.64 (m, 2H), 3.61-3.58 (m, 4H), 3.50-3.48 (m, 2H), 2.44 (s, 3H), 2.07 (d, J=7.5 Hz, 1H), 1.95-1.89 (m, 1H), 1.44 (d, J=6.0 Hz, 2H), 1.40-1.37 (m, 1H), 1.35-1.33 (m, 1H), 1.28-1.16 (m, 8H), 0.95 (s, 9H).

### Example A46. 2-amino-N—((S)-6-(6-((2-(2-(2-(2-(5-ethynyl-2-(((28,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamoyl)pyridin-3-yl)-2,3-carboxamide

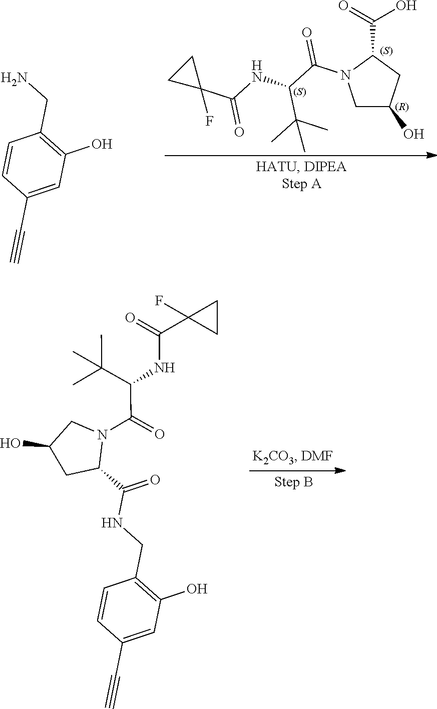

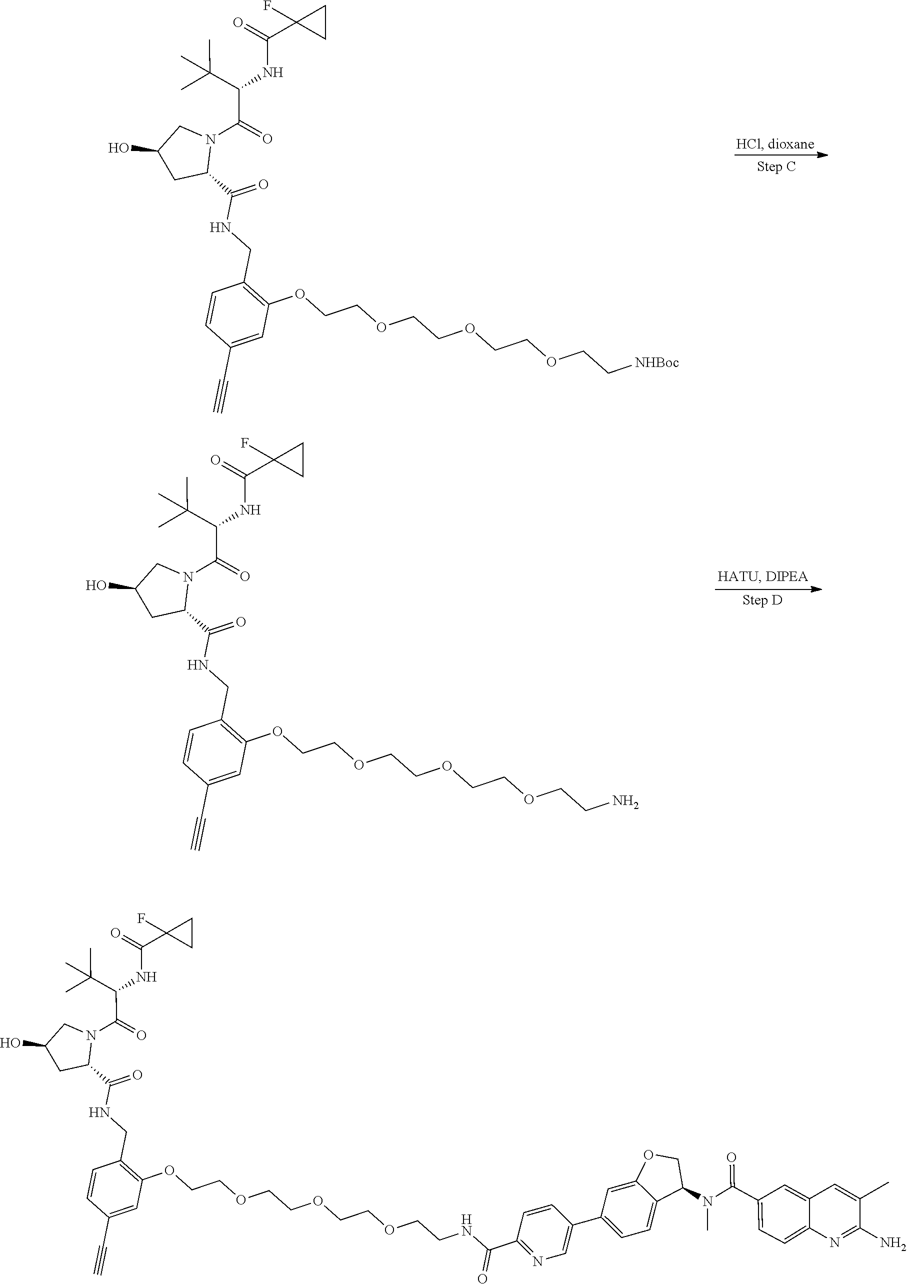

Step A: (2S,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide

To a stirred mixture of (2S,4R)-1-((S)-2-(1-fluorocyclopropane-1-carboxamido)-3,3-dimethylbutanoyl)-4-hydroxypyrrolidine-2-carboxylic acid (WO 2023/278402, 100 mg, 303 μmol, 1.0 eq.) in DMF (3 mL) were added 2-(aminomethyl)-5-ethynylphenol (54 mg, 363 μmol, 1.2 eq.), HATU (173 mg, 454 μmol, 1.5 eq.) and DIEA (157 μL, 908 μmol, 3.0 eq.). The resulting mixture was stirred at room temperature for 1 hour. The reaction mixture was poured into water (30 mL) and then extracted with EtOAc (30 mL×3). The combined organic layers were washed with brine, dried over anhydrous Na₂SO₄ and concentrated. The residue was purified by prep-HPLC(5-55% MeOH in H₂O) to give (28,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide (80.0 mg, 58% yield) as a yellow solid.

LC-MS (ESI): mass calcd, for C₂₄H₃₀FN₃O₅, 459.52; m/z found, 460.4 [M+H]⁺.

### Step B-D

Example A46 was synthesized following the same procedures as described in Example A13 (5.4 mg, 7% yield over 3 steps) as a white solid. LC-MS (ESI): mass calcd, for C₅₈H₆₇FN₈O₁₁, 1071.2; m/z found, 1072.3 [M+H]⁺. ¹H NMR (400 MHz, DMSO-d₆) δ 8.94 (s, 1H), 8.76-8.68 (m, 1H), 8.47 (t, J=5.9 Hz, 1H), 8.28 (dd, J=8.2, 1.9 Hz, 1H), 8.09 (d, J=8.1 Hz, 1H), 7.88 (s, 1H), 7.82 (s, 1H), 7.61-7.52 (m, 3H), 7.40 (d, J=7.6 Hz, 1H), 7.35 (s, 1H), 7.32-7.26 (m, 2H), 7.03 (s, 1H), 6.97 (d, J=7.7 Hz, 1H), 6.74 (s, 1H), 5.17 (d, J=3.4 Hz, 1H), 4.85-4.73 (m, 1H), 4.72-4.67 (m, 1H), 4.59 (d, J=9.3 Hz, 1H), 4.49 (t, J=8.2 Hz, 1H), 4.34 (s, 1H), 4.29-4.24 (m, 1H), 4.20-4.15 (m, 1H), 4.13-4.11 (m, 2H), 3.77-3.74 (m, 2H), 3.63-3.59 (m, 4H), 3.58-3.54 (m, 8H), 3.51-3.47 (m, 4H), 2.68 (s, 3H), 2.24 (s, 3H), 2.12-2.04 (m, 1H), 1.94-1.85 (m, 1H), 1.41-1.31 (m, 2H), 1.25-1.20 (m, 2H), 0.95 (s, 9H).

### Example A47. 2-amino-3-cyclopropyl-N—((S)-6-(6-((2-(2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)carbamoyl)pyridin-3-yl)-2,3-dihydrobenzofuran-3-yl)-N-methylquinoline-6-carboxamide

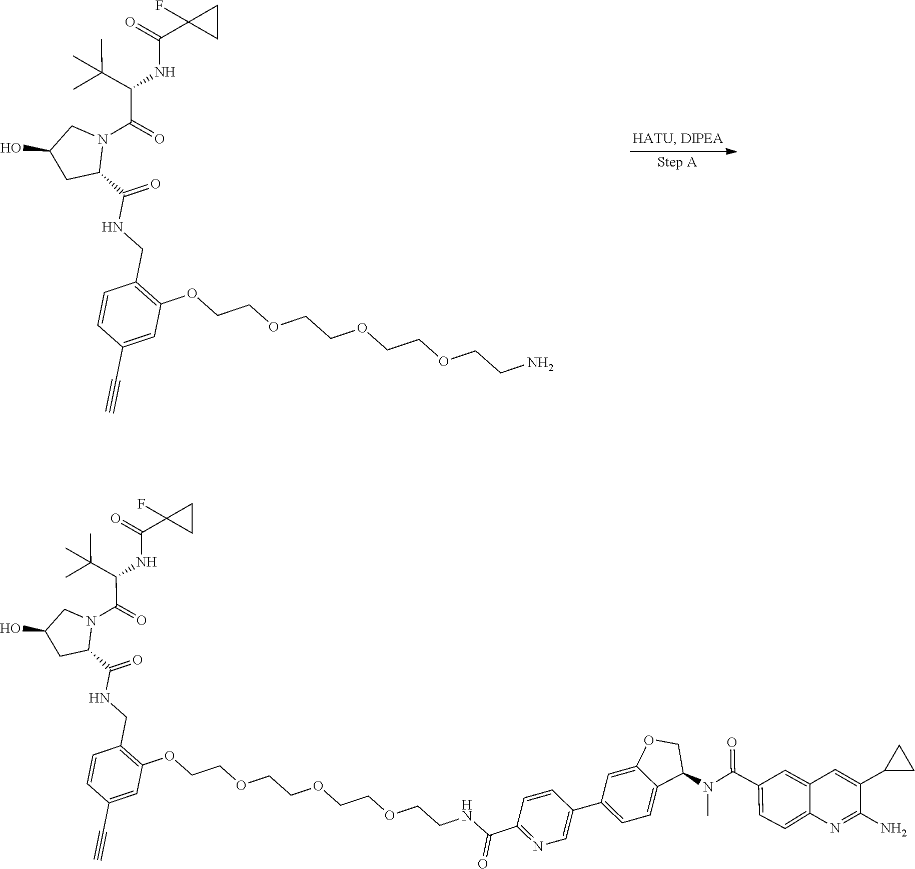 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCN)c1 ; C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3ccc4nc(N)c(C5CC5)cc4c3)cn2)c1]

Example A47 was synthesized following the same procedures as described in Example A13 (7.6 mg, 6% yield) as a white solid. LC-MS (ESI): mass calcd, for C₆₀H₆₉FN₈O₁₁, 1096.5; m/z found, 1097.4 [M+H]⁺. ¹H NMR (400 MHz, DMSO-d₆) δ 8.94 (d, J=1.8 Hz, 1H), 8.74-8.69 (m, 1H), 8.47 (t, J=5.9 Hz, 1H), 8.28 (dd, J=8.2, 2.2 Hz, 1H), 8.09 (d, J=8.2 Hz, 1H), 7.84 (s, 1H), 7.78 (s, 1H), 7.62-7.58 (m, 1H), 7.56-7.50 (m, 2H), 7.40 (d, J=8.0 Hz, 1H), 7.35 (s, 1H), 7.32-7.25 (m, 2H), 7.03 (s, 1H), 6.98-6.95 (m, 1H), 5.17 (d, J=3.3 Hz, 1H), 4.76 (s, 1H), 4.69-4.65 (m, 1H), 4.59 (d, J=9.2 Hz, 1H), 4.49 (t, J=8.2 Hz, 1H), 4.34 (s, 1H), 4.29-4.24 (m, 1H), 4.20-4.15 (m, 1H), 4.13-4.11 (m, 2H), 3.77-3.73 (m, 2H), 3.67-3.62 (m, 2H), 3.61-3.57 (m, 4H), 3.57-3.56 (m, 2H), 3.55-3.54 (m, 4H), 3.52-3.46 (m, 4H), 2.67 (s, 3H), 2.12-2.04 (m, 1H), 1.93-1.83 (m, 2H), 1.40-1.32 (m, 2H), 1.24-1.19 (m, 2H), 1.02-0.98 (m, 2H), 0.95 (s, 9H), 0.69 (d, J=3.9 Hz, 2H).

### Example A48. 2-amino-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N-isopropyl-3-methylquinoline-6-carboxamide

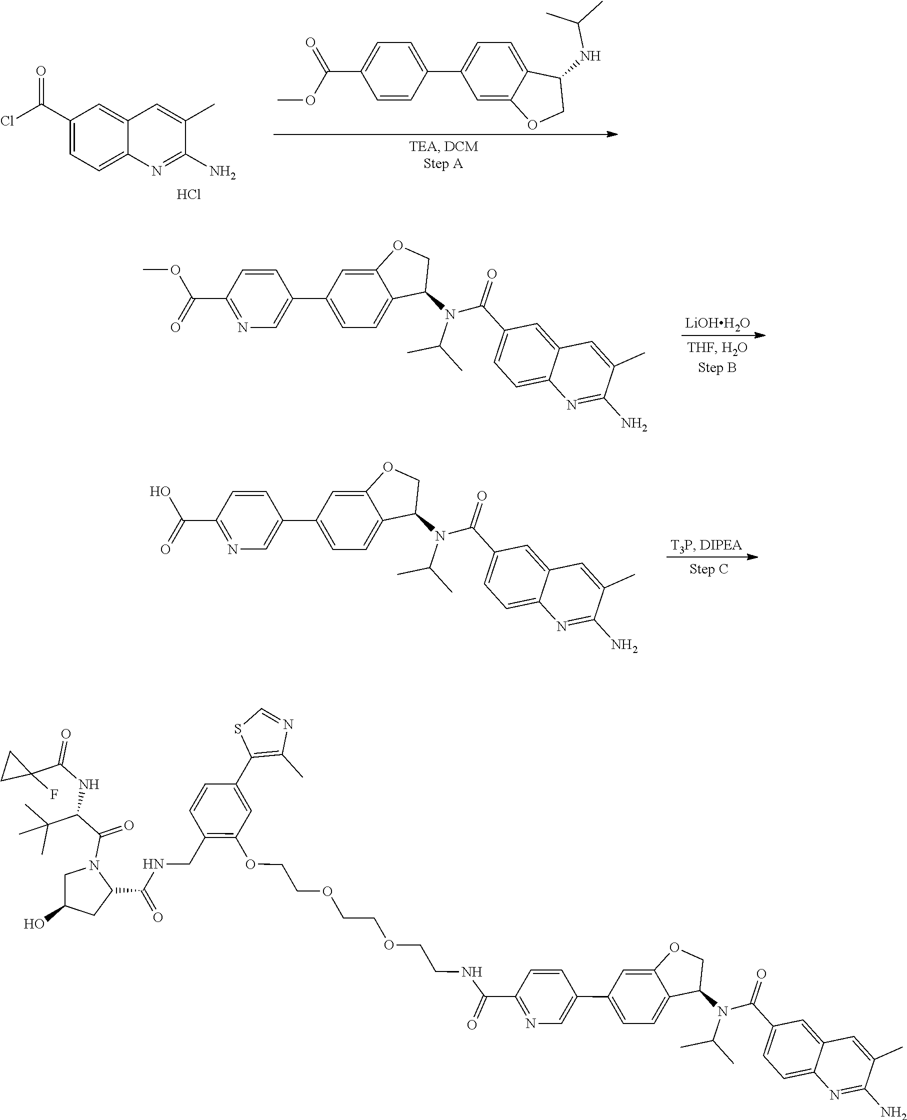

Example A48 was synthesized following the same procedures as described in Example A45 (8 mg, 4% yield over 3 steps) as a white solid. LC-MS (ESI): mass calcd, for C₆₀H₇₀FN₉O₁₀S, 1127; m/z found, 1128.2 [M+H]⁺.

### Example A49. 2-amino-3-cyclopropyl-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methylquinoline-6-carboxamide

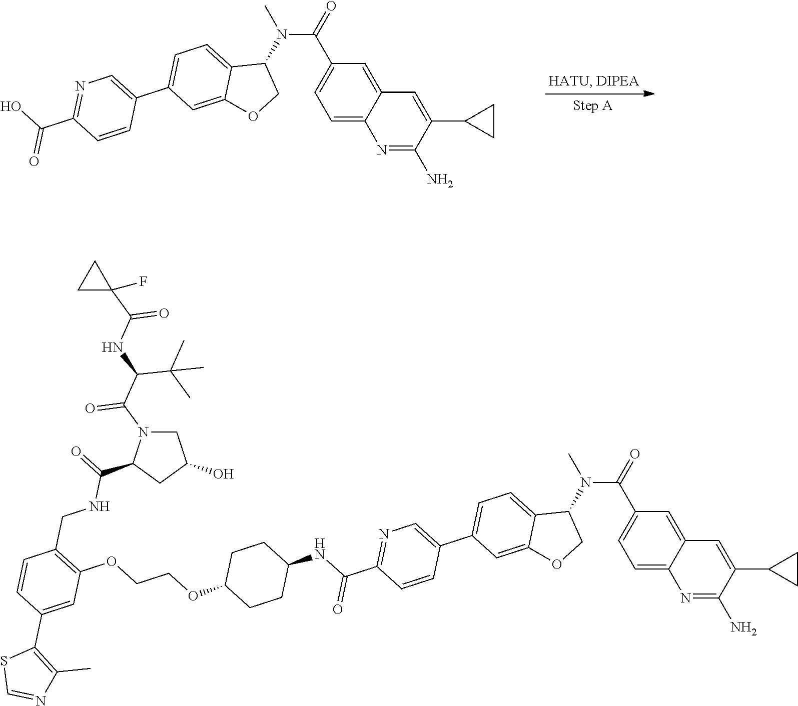 [SMILES: CN(C(=O)c1ccc2nc(N)c(C3CC3)cc2c1)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CC6)cc5c4)cn3)CC2)c1]

Example A49 was synthesized following the same procedures as described in Example A29 (6.7 mg, 15% yield) as a white solid. LC-MS (ESI): mass calcd, for C₆₂H₇₀FN₉O₉S, 1135.50; m/z found, 1136.3 [M+H]⁺. ¹H NMR (400 MHz, DMSO-d₆) δ 8.99 (s, 1H), 8.92 (d, 1H), 8.52-8.44 (m, 2H), 8.30-8.23 (m, 1H), 8.08 (d, J=8.3 Hz, 1H), 7.80 (s, 1H), 7.71 (s, 1H), 7.57-7.48 (m, 3H), 7.44-7.37 (m, 2H), 7.35-7.32 (m, 1H), 7.31-7.27 (m, 1H), 7.07-7.02 (m, 1H), 7.00-6.94 (m, 1H), 6.68 (s, 2H), 5.17 (d, J=3.5 Hz, 1H), 4.80-4.64 (m, 2H), 4.59 (d, J=9.4 Hz, 1H), 4.52 (t, J=8.1 Hz, 1H), 4.41-4.23 (m, 3H), 4.22-4.10 (m, 3H), 3.83-3.76 (m, 3H), 3.68-3.57 (m, 2H), 2.67 (s, 3H), 2.47 (s, 3H), 2.11-2.01 (m, 3H), 1.91-1.77 (m, 4H), 1.57-1.47 (m, 2H), 1.41-1.12 (m, 8H), 0.95 (s, 9H), 0.68-0.63 (m, 2H).

### Example A50. 4-amino-N-ethyl-N—((S)-6-(6-((2-(2-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-1,3-dihydrofuro[3,4-c][1,7]naphthyridine-8-carboxamide

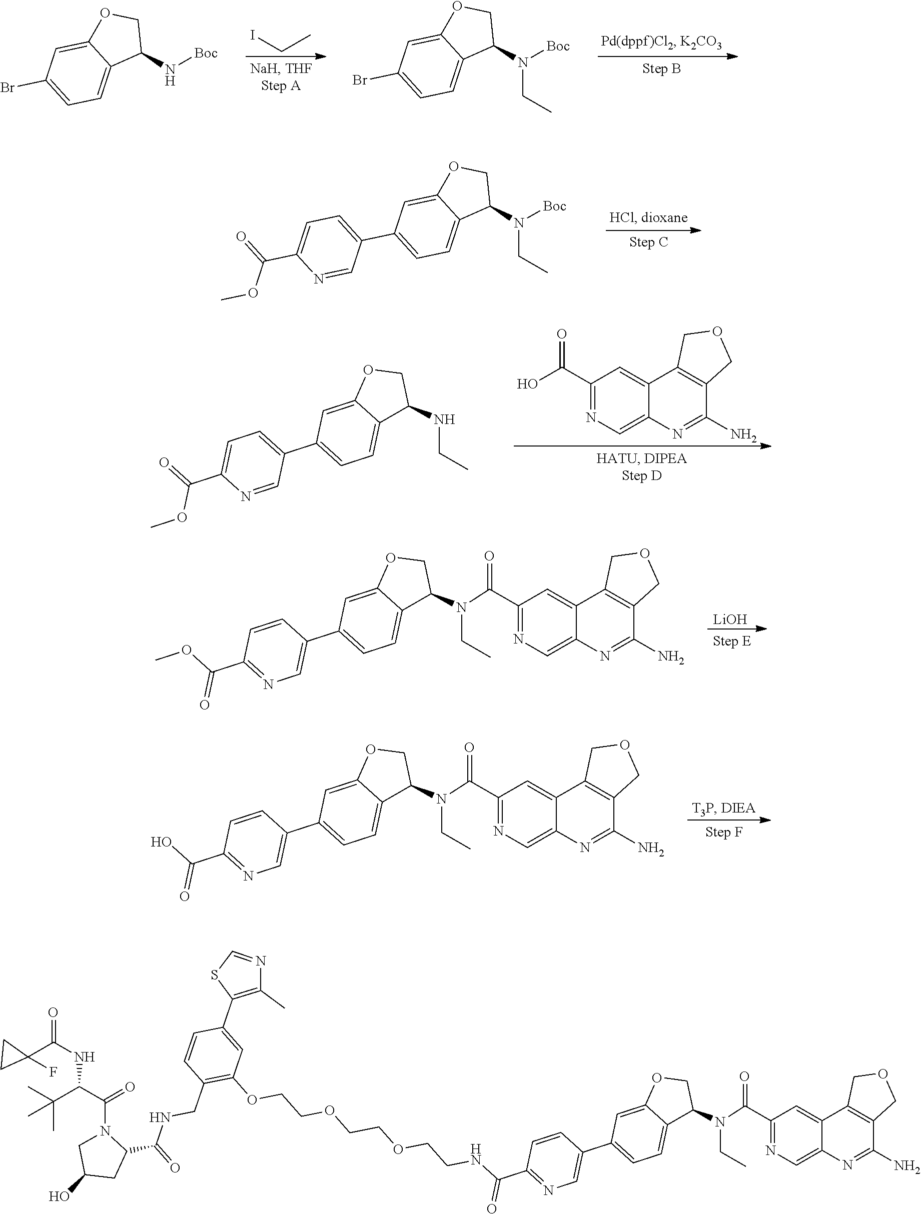

Step A: (S)-2-amino-N-(6-bromo-2,3-dihydrobenzofuran-3-yl)-N,3-dimethylquinoline-6-carboxamide

To a solution of tert-butyl(S)-(6-bromo-2,3-dihydrobenzofuran-3-yl)carbamate (500 mg, 1.59 mmol, 1.0 eq.) in DMF (5 mL) was added NaH (95.5 mg, 2.39 mmol, 1.5 eq.) at 0° C. The mixture was stirred for 1 hour under N₂ atmosphere. Then Iodoethane (372 mg, 2.39 mmol, 1.5 eq.) was added at 0° C. The mixture was stirred at room temperature for 1 hour. The mixture was quenched by addition to ice-water and extracted with EtOAc (10 mL×3). The combined organic layers were washed with brine, dried over anhydrous Na₂SO₄ and concentrated under reduced pressure. The residue was purified by flash chromatographer (eluent, 0~25% EtOAc in PE) to give tert-butyl(S)-(6-bromo-2,3-dihydrobenzofuran-3-yl)(ethyl)carbamate (510 mg, 94% yield) as a white solid.

LC-MS (ESI): mass calcd, for C₁₅H₂₀BrNO3, 341/343; m/z found, 286.1/288.1 [M+H−56]⁺.

Step B: methyl(S)-5-(3-((tert-butoxycarbonyl)(ethyl)amino)-2,3-dihydrobenzofuran-6-yl)picolinate

To a mixture of tert-butyl(S)-(6-bromo-2,3-dihydrobenzofuran-3-yl)(ethyl)carbamate (480 mg, 1.4 mmol, 1.0 eq.), methyl 5-(4,4,5,5-tetramethyl-1,3,2-dioxaborolan-2-yl)picolinate (554 mg, 2.1 mmol, 1.5 eq.) and K₂CO₃ (582 mg, 4.21 mmol, 3.0 eq.) in 1,4-dioxane (6.0 mL) and H₂O (0.3 mL) was added Pd(dppf)Cl₂ (103 mg, 140 μmol, 0.1 eq.) under nitrogen atmosphere. Then the mixture was stirred at 90° C. for 3 hours. The mixture was poured into water (20 mL) and extracted with EtOAc (20 mL×3). The combined organic layers were washed with brine, dried over anhydrous Na₂SO₄ and concentrated under reduced pressure. The residue was purified by flash chromatographer (eluent, 0~50% EtOAc in PE) to methyl(S)-5-(3-((tert-butoxycarbonyl)(ethyl)amino)-2,3-dihydrobenzofuran-6-yl)picolinate (510 mg, 91% yield) as a yellow solid.

LC-MS (ESI): mass calcd, for C₂₂H₂₆N₂O₅, 398.18; m/z found, 399.3 [M+H]⁺.

Step C: methyl(S)-5-(3-(ethylamino)-2,3-dihydrobenzofuran-6-yl)picolinate

To a solution of methyl(S)-5-(3-((tert-butoxycarbonyl)(ethyl)amino)-2,3-dihydrobenzofuran-6-yl)picolinate (510 mg, 1.28 mmol, 1.0 eq.) in HCl/1,4-Dioxane (6 mL, 4 mol/L) at 0° C. The reaction mixture was stirred for 1 hour at room temperature under N₂ atmosphere. The mixture was concentrated by vacuum to give methyl(S)-5-(3-(ethylamino)-2,3-dihydrobenzofuran-6-yl)picolinate (380 mg, 99% yield) as a white solid.

LC-MS (ESI): mass calcd, for C₁₇H₁₈N₂O₃, 298.13; m/z found, 299.1 [M+H]⁺.

### Step D-F

Example A50 was synthesized following the same procedures as described in Example A9 (6.4 mg, 9% yield over 3 steps) as a white solid. LC-MS (ESI): mass calcd. for C₅₉H₆₇FN₁₀O₁₁S, 1142.5; m/z found, 1143.2 [M+H]⁺. ¹H NMR (400 MHz, DMSO-d₆) δ 8.95 (s, 1H), 8.93-8.84 (m, 2H), 8.71 (t, J=5.8 Hz, 1H), 8.48 (t, J=6.0 Hz, 1H), 8.25 (d, J=7.6 Hz, 1H), 8.07 (d, J=8.2 Hz, 1H), 7.90-7.76 (m, 1H), 7.66-7.46 (m, 1H), 7.41-7.26 (m, 4H), 7.21-7.07 (m, 2H), 7.01 (s, 1H), 6.95 (d, J=7.8 Hz, 1H), 6.01 (d, J=80.0 Hz, 1H), 5.43-5.35 (m, 2H), 5.05 (s, 2H), 4.74-4.56 (m, 3H), 4.51 (t, J=8.2 Hz, 1H), 4.36-4.33 (m, 1H), 4.29-4.22 (m, 1H), 4.19-4.15 (m, 2H), 3.82-3.78 (m, 2H), 3.66-3.63 (m, 3H), 3.61-3.57 (m, 5H), 3.51-3.47 (m, 4H), 3.21-3.14 (m, 2H), 2.43 (s, 3H), 2.11-2.04 (m, 1H), 1.96-1.88 (m, 1H), 1.38-1.33 (m, 2H), 1.24-1.19 (m, 2H), 1.09-0.96 (m, 3H), 0.95 (s, 9H).

### Example A51. 2-amino-3-cyclopropyl-N-(cyclopropylmethyl)-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydroben zofuran-3-yl)quinoline-6-carboxamide

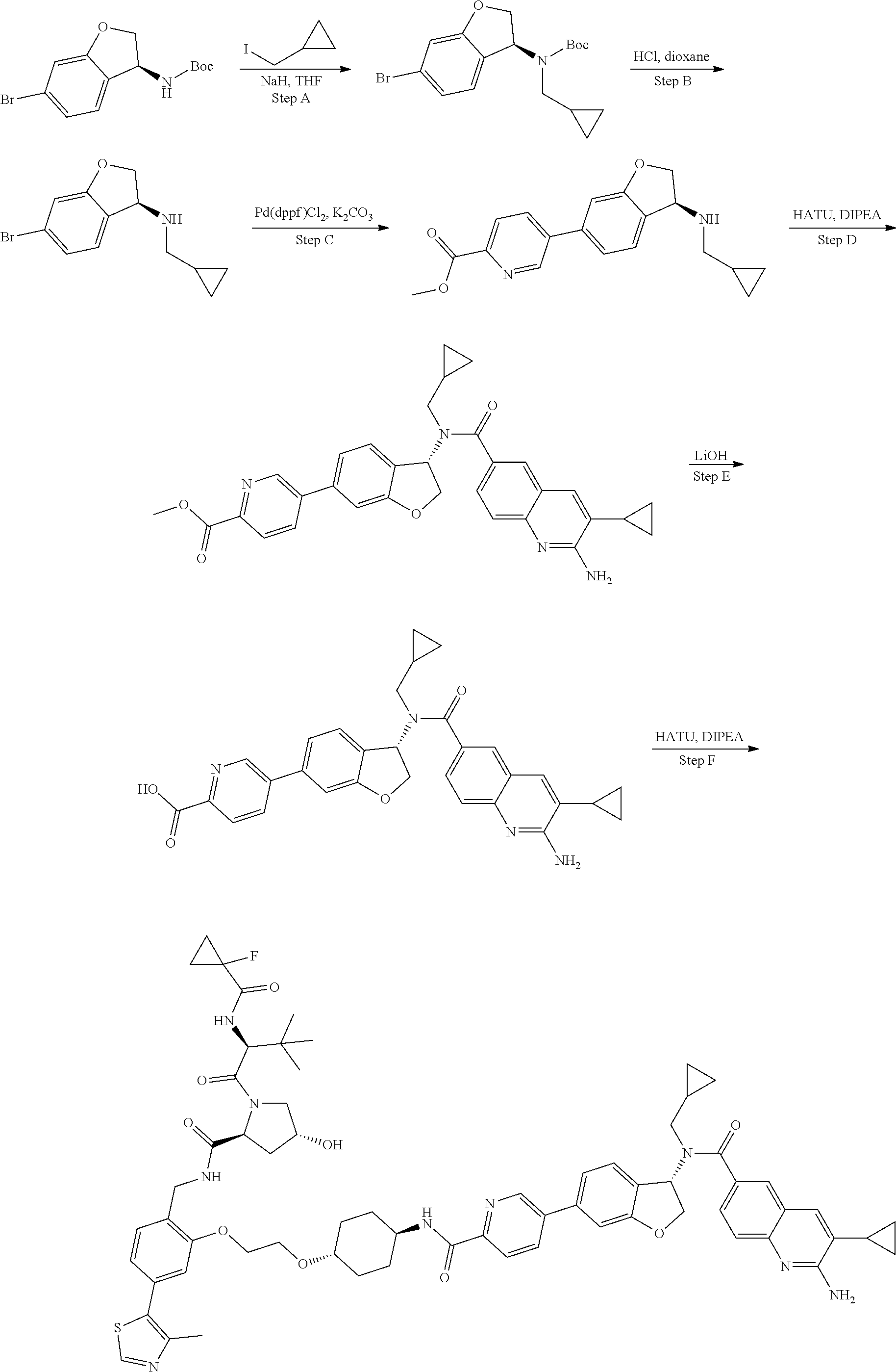

Step A: tert-butyl(S)-(6-bromo-2,3-dihydrobenzofuran-3-yl)(cyclopropylmethyl)carbamate

To a solution of tert-butyl (R)-(6-bromo-2,3-dihydrobenzofuran-3-yl)carbamate (100 mg, 318 μmol, 1.0 eq.) in DMF (5 mL) was added NaH (16 mg, 637 μmol, 2.0 eq., 60% w/w) at 0° C. The mixture was stirred at room temperature for 1 hour and then (iodomethyl)cyclopropane (87 mg, 477 μmol, 1.5 eq.) was added to the solution. The mixture was diluted with water (30 mL) and extracted with EtOAc (20 mL×3). The combined organic layers were washed with brine, dried over anhydrous Na₂SO₄ and concentrated under reduced pressure. The residue was purified by flash chromatographer (eluent, 0~20% EtOAc in PE) to afford tert-butyl (R)-(6-bromo-2,3-dihydrobenzofuran-3-yl)(cyclopropylmethyl)carbamate (80 mg, 68% yield) as a yellow solid.

LC-MS (ESI): mass calcd, for C17H22BrNO3, 367.1; m/z found, 368.1 [M+H]⁺.

Step B: (S)-6-bromo-N-(cyclopropylmethyl)-2,3-dihydrobenzofuran-3-amine

To a solution of tert-butyl (R)-(6-bromo-2,3-dihydrobenzofuran-3-yl)(cyclopropylmethyl)carbamate (80 mg, 0.22 mmol, 1.0 eq.) in dioxane (5 mL) was added HCl/dioxane (1 mL, 4 mol/L). The mixture was stirred at room temperature for 1 hour. The mixture was concentrated by vacuum to afford(S)-6-bromo-N-(cyclopropylmethyl)-2,3-dihydrobenzofuran-3-amine (80 mg crude, HCl salt) as a yellow solid.

LC-MS (ESI): mass calcd, for C12H14BrNO, 267.1; m/z found, 268.1 [M+H]⁺.

Step C: methyl(S)-5-(3-((cyclopropylmethyl)amino)-2,3-dihydrobenzofuran-6-yl)picolinate

To a solution of(S)-6-bromo-N-(cyclopropylmethyl)-2,3-dihydrobenzofuran-3-amine (80 mg, 0.3 mmol, 1.0 eq.), methyl 5-(4,4,5,5-tetramethyl-1,3,2-dioxaborolan-2-yl)picolinate (80 mg, 0. 3 mmol, 1.0 eq.) and K₂CO₃ (83 mg, 0.6 mmol, 2.0 eq.) in dioxane (4 mL) and water (0.4 mL) was added Pd(dppf)Cl₂ (22 mg, 0.03 mmol, 0.1 eq,). The mixture was stirred at 90° C. for 3 hours. The mixture was diluted with water (30 mL) and extracted with EtOAc (20 mL×3). The combined organic layers were washed with brine, dried over anhydrous Na₂SO₄ and concentrated under reduced pressure. The residue was purified by flash chromatographer (eluent, 0~4% MeOH in DCM) to give methyl(S)-5-(3-((cyclopropylmethyl)amino)-2,3-dihydrobenzofuran-6-yl)picolinate (40 mg, 60% yield) as a white solid.

LC-MS (ESI): mass calcd, for C₁₉H₂₀N₂O₃, 324.1; m/z found, 325.1 [M+H]⁺.

### Step D-F

Example A51 was synthesized following the same procedures as described in Example A29 (11 mg, 15% yield over 3 steps) as a white solid. LC-MS (ESI): mass calcd. for C₆₅H₇₄FN₉O₉S, 1175.53; m/z found, 1176.4 [M+H]⁺.

Example A52. 4-amino-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N,1,3-trimethyl-1H-pyrazolo[4,3-c]quinoline-8-carboxamide

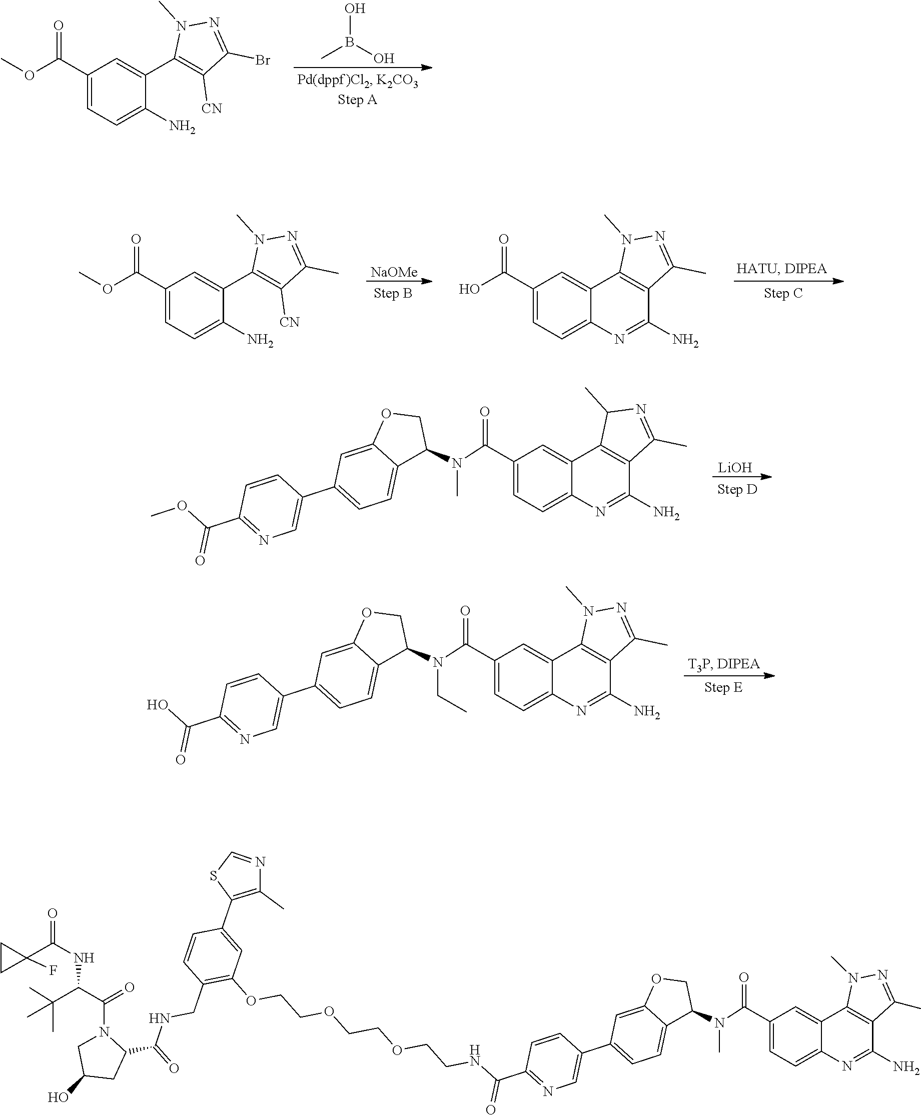

Step A: methyl 4-amino-3-(4-cyano-1,3-dimethyl-1H-pyrazol-5-yl)benzoate

To a mixture of methyl 4-amino-3-(3-bromo-4-cyano-1-methyl-1H-pyrazol-5-yl)benzoate (100 mg, 298 μmol, 1.0 eq.), methylboronic acid (89.3 mg, 1.49 mmol, 5.0 eq.) and K₃PO₄ (190 mg, 895 μmol, 3.0 eq.) in 1,4-dioxane (2.0 mL) and H₂O (0.1 mL) was added Pd(dppf)Cl₂ (21.8 mg, 29.8 μmol, 0.1 eq.) at room temperature and stirred at 90° C. for 1 hour. The cooled mixture was diluted with EtOAc (20 mL×3) and washed with H₂O and brine. The organic layers were dried over anhydrous Na₂SO₄, filtered and concentrated under reduced pressure to dryness. The residue was purified by flash chromatographer (eluent, 0~3% MeOH in DCM) to give the methyl 4-amino-3-(4-cyano-1,3-dimethyl-1H-pyrazol-5-yl)benzoate (30 mg, 37% yield) as a white solid.

LC-MS (ESI): mass calcd, for C₁₄H₁₄N₄O₂, 270; m/z found, 271.1 [M+H]⁺.

Step B: 4-amino-1,3-dimethyl-1H-pyrazolo[4,3-c]quinoline-8-carboxylic acid

To a solution of methyl 4-amino-3-(4-cyano-1,3-dimethyl-1H-pyrazol-5-yl)benzoate (30 mg, 111 μmol, 1.0 eq.) in MeOH (1.00 mL) was added Sodium methoxide (934 mg, 1.00 mL, 33% Wt, 51.4 eq, 5.71 mmol) under nitrogen atmosphere. Then the mixture was stirred at 70° C. for 5 hours. The cooled mixture was concentrated under reduced pressure to dryness to give the 4-amino-1,3-dimethyl-1H-pyrazolo[4,3-c]quinoline-8-carboxylic acid (25 mg, 88% yield) as a white solid which was used into next step without further purification.

LC-MS (ESI): mass calcd, for C₁₃H₁₂N₄O₂, 256; m/z found, 257.0 [M+H]⁺.

### Step C-E

Example A52 was synthesized following the same procedures as described in Example A41 (5.4 mg, 4% yield over 3 steps) as a white solid. LC-MS (ESI): mass calcd. for C₆₀H₆₈FN₁₁O₁₀S, 1153.4; m/z found, 1154.2 [M+H]⁺. ¹H NMR (400 MHz, DMSO-d₆) δ 8.94 (s, 1H), 8.90 (s, 1H), 8.70 (t, J=5.7 Hz, 1H), 8.48 (t, J=5.9 Hz, 1H), 8.31 (s, 1H), 8.25 (d, J=8.1 Hz, 1H), 8.07 (d, J=8.1 Hz, 1H), 7.75-7.62 (m, 2H), 7.55 (s, 1H), 7.41-7.34 (m, 2H), 7.32-7.26 (m, 2H), 7.00 (s, 1H), 6.94 (d, J=7.7 Hz, 1H), 4.74 (s, 2H), 4.58 (d, J=9.1 Hz, 1H), 4.51 (t, J=8.2 Hz, 1H), 4.37-4.31 (m, 4H), 4.28 (d,)=6.0 Hz, 1H), 4.22 (d, J=5.6 Hz, 1H), 4.18-4.15 (m, 2H), 3.81-3.78 (m, 2H), 3.68-3.62 (m, 4H), 3.61-3.56 (m, 6H), 3.51-3.47 (m, 5H), 2.70 (s, 3H), 2.61 (s, 3H), 2.42 (s, 3H), 2.11-2.04 (m, 1H), 1.94-1.87 (m, 1H), 1.36-1.30 (m, 2H), 1.23-1.18 (m, 2H), 0.94 (s, 9H).

### Example A53. N1-(7-amino-1-methyl-1H-pyrazolo[3,4-c]pyridin-4-yl)-N2-((S)-6-(6-(((1S,4S)-4-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N2-methyloxalamide

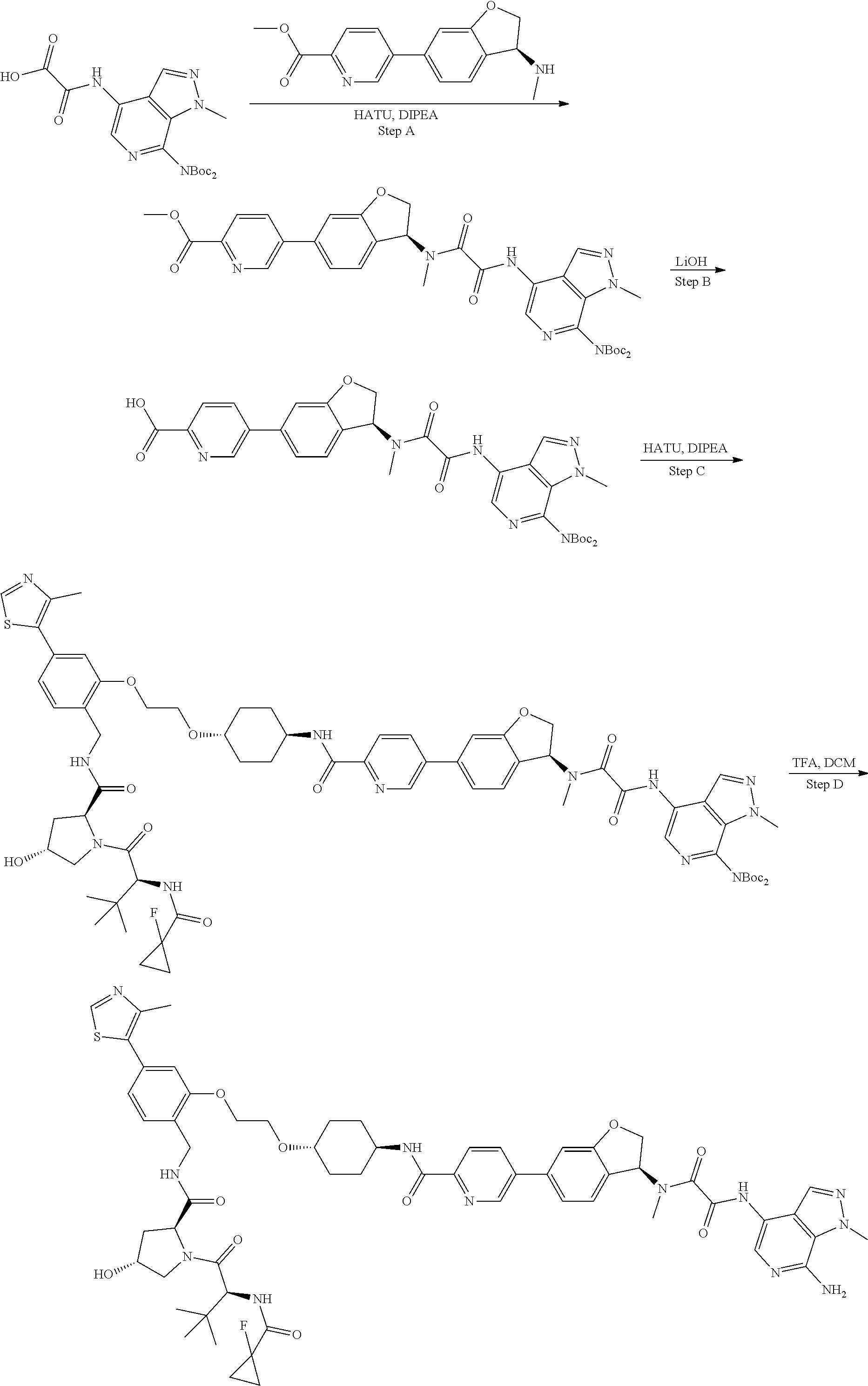

Example A53 was synthesized following the same procedures as described in Example A44 (1.8 mg, 3% yield over 4 steps) as a white solid. LC-MS (ESI): mass calcd. for C₅₈H₆₇FN₁₂O₁₀S, 1142.5; m/z found, 1143.7 [M+H]⁺. ¹H NMR (400 MHz, DMSO-d₆) δ 8.98 (s, 1H), 8.92 (d, J=4.7 Hz, 1H), 8.52-8.44 (m, 2H), 8.34-8.19 (m, 2H), 8.10-7.98 (m, 2H), 7.60 (d, J=7.7 Hz, 1H), 7.51-7.43 (m, 1H), 7.43-7.32 (m, 3H), 7.29 (d, J=7.7 Hz, 1H), 7.05 (s, 1H), 6.97 (d, J=8.0 Hz, 1H), 4.85-4.61 (m, 2H), 4.59 (d, J=9.3 Hz, 1H), 4.52 (t, J=8.1 Hz, 1H), 4.37-4.33 (m, 4H), 4.32-4.27 (m, 1H), 4.27-4.20 (m, 1H), 4.19-4.13 (m, 2H), 3.88-3.74 (m, 3H), 3.70-3.47 (m, 2H), 3.43-3.38 (m, 2H), 2.75 (s, 1H), 2.65 (s, 2H), 2.47 (s, 3H), 2.10-1.93 (m, 6H), 1.88-1.82 (m, 2H), 1.61-1.39 (m, 4H), 1.38-1.29 (m, 4H), 1.22-1.16 (m, 2H), 0,95 (s, 9H).

### Example A54. 2-amino-N-(cyclopropylmethyl)-N—((S)-6-(6-(((18,4S)-4-(2-(2-(((28,4R)-1-((S)-2-(1-fluorocyclopropane-1-carboxamido)-3,3-dimethylbutanoyl)-4-hydroxypyrrolidine -2-carboxamido)methyl)-5-(4-methylthiazol-5-yl)phenoxy)ethoxy)cyclohexyl)carbamoyl) pyridin-3-yl)-2,3-dihydrobenzofuran-3-yl)-3-methylquinoline-6-carboxamide

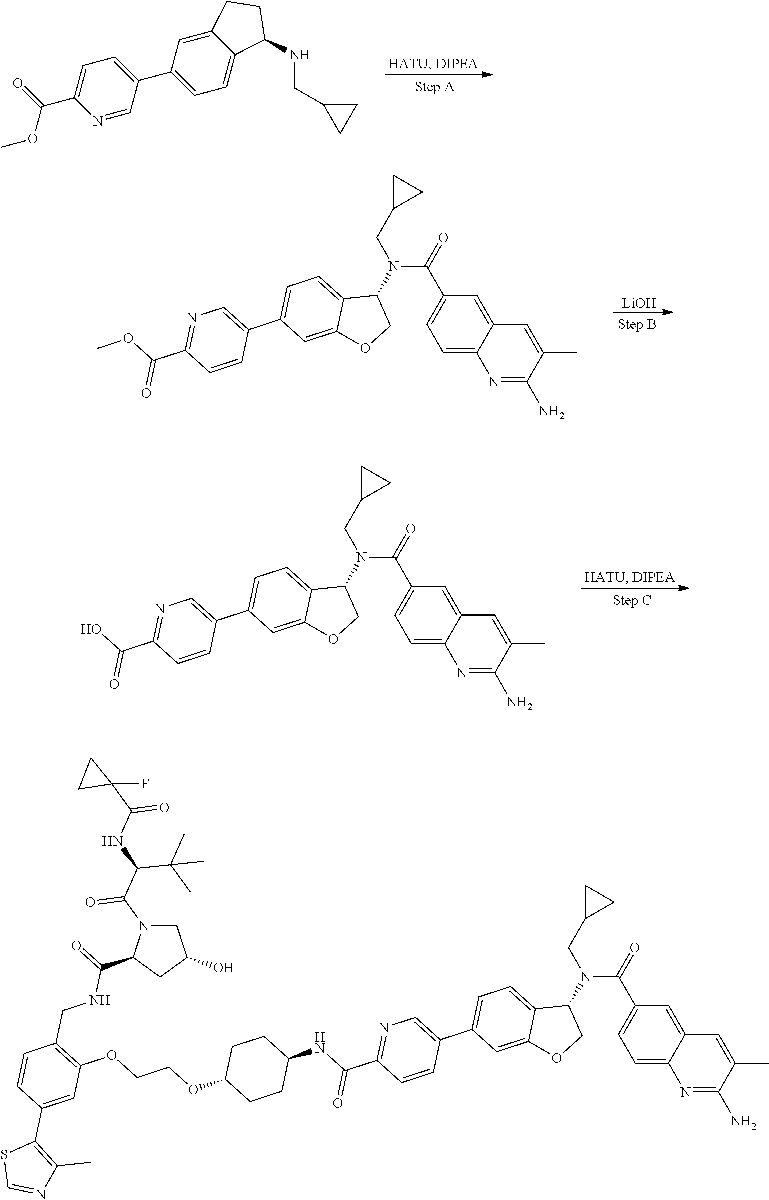

Example A54 was synthesized following the same procedures as described in Example A51 (2.5 mg, 13% yield over 3 steps) as a white solid. LC-MS (ESI): mass calcd. for C₆₃H₇₂FN₉O₉S, 1149.5; m/z found, 1150.4 [M+H]⁺. ¹H NMR (400 MHz, DMSO-d₆) δ 8.99 (s, 1H), 8.92 (s, 1H), 8.51-8.45 (m, 2H), 8.27 (d, J=10.3 Hz, 1H), 8.08 (d, J=8.2 Hz, 1H), 7.81 (s, 1H), 7.60-7.56 (m, 2H), 7.43-7.36 (m, 2H), 7.32-7.27 (m, 2H), 7.05 (s, 1H), 6.97 (d, J=9.1 Hz, 1H), 4.76-4.70 (m, 1H), 4.60 (d, J=8.7 Hz, 1H), 4.55-4.46 (m, 1H), 4.38-4.34 (m, 1H), 4.33-4.27 (m, 1H), 4.27-4.20 (m, 1H), 4.20-4.15 (m, 2H), 3.84-3.77 (m, 3H), 3.67-3.57 (m, 3H), 3.51-3.45 (m, 4H), 2.47 (s, 3H), 2.25 (s, 3H), 2.11-2.01 (m, 4H), 1.89-1.84 (m, 2H), 1.60-1.54 (m, 2H), 1.52-1.47 (m, 3H), 1.35-1.31 (m, 4H), 1.23-1.20 (m, 2H), 0.95 (s, 9H), 0.40-0.26 (m, 2H).

### Example A55. 4-amino-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,1,3-trimethyl-1H-pyrazolo[4,3-c]quinoline-8-carboxamide

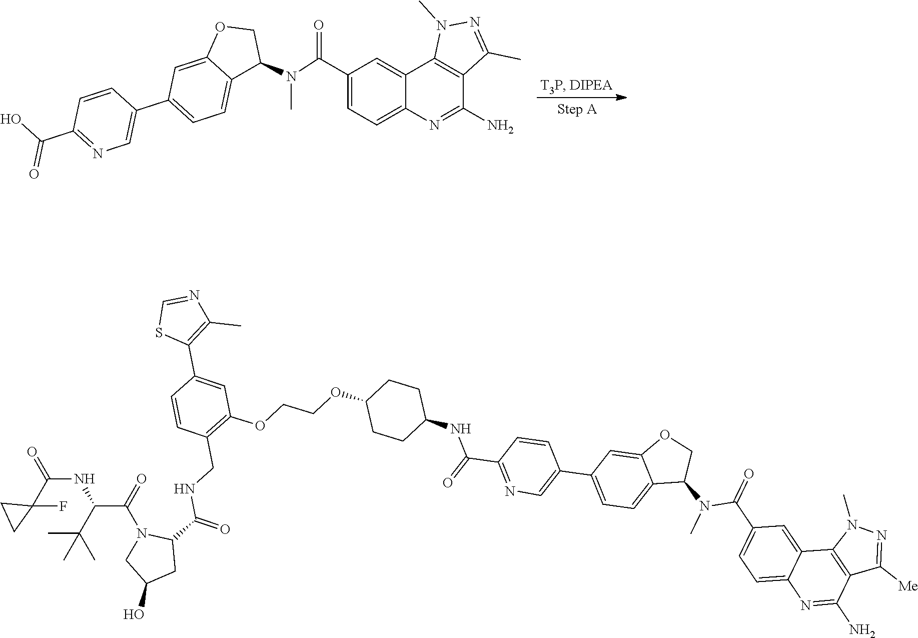

Example A55 was synthesized following the same procedures as described in Example A52 (3.5 mg, 15% yield) as a white solid. LC-MS (ESI): mass calcd, for C₆₂H₇₀FN₁₁O₉S, 1163.51; m/z found, 1164.5 [M+H]⁺. ¹H NMR (400 MHz, DMSO-d₆) δ 9.04-8.86 (m, 2H), 8.55-8.43 (m, 2H), 8.32-8.27 (m, 1H), 8.19-8.04 (m, 1H), 7.67-7.60 (m, 2H), 7.43-7.37 (m, 2H), 7.35-7.24 (m, 2H), 7.05 (d, J=4.6 Hz, 1H), 6.97 (d, J=8.8 Hz, 1H), 6.63 (s, 1H), 4.78-4.72 (m, 1H), 4.60 (d, J=8.9 Hz, 1H), 4.54-4.50 (m, 1H), 4.39-4.32 (m, 4H), 4.31-4.23 (m, 2H), 4.20-4.14 (m, 3H), 3.82-3.76 (m, 4H), 3.66-3.61 (m, 3H), 2.72 (s, 3H), 2.61 (s, 3H), 2.47 (s, 3H), 2.09-2.03 (m, 3H), 1.96-1.93 (m, 1H), 1.88-1.82 (m, 2H), 1.79-1.72 (m, 1H), 1.65-1.57 (m, 1H), 1.53-1.50 (m, 1H), 1.41-1.38 (m, 1H), 1.35-1.31 (m, 2H), 1.23-1.20 (m, 2H), 0.96 (s, 9H).

### Example A56. N1-(7-amino-1-methyl-1H-pyrazolo[3,4-c]pyridin-4-yl)-N2-(cyclopropylmethyl)-N2-((S)-6-(6-(((1S,4S)-4-(2-(2-(((28,4R)-1-((S)-2-(1-fluorocyclopropane -1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)oxalamide

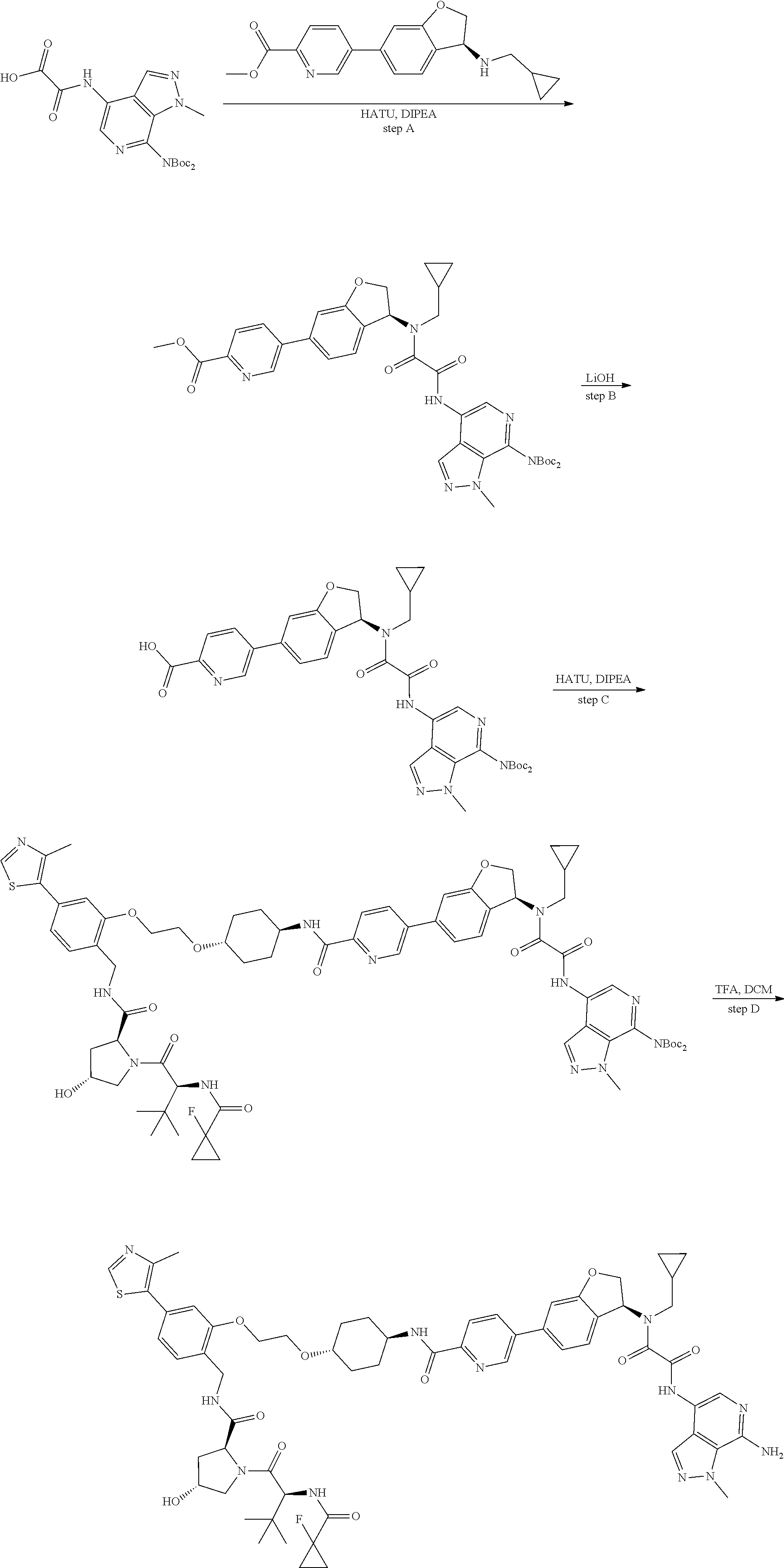

Example A56 was synthesized following the same procedures as described in Example A44 (5.4 mg, 7% yield over 4 steps) as a white solid. LC-MS (ESI): mass calcd. for C₆₁H₁₇FN₁₂O₁₀S, 1182.5; m/z found, 1183.3 [M+H]⁺. ¹H NMR (400 MHz, DMSO-d₆) δ 10.64 (d, J=25.5 Hz, 1H), 8.89 (s, 1H), 8.82 (d, J=11.0 Hz, 1H), 8.42-8.37 (m, 2H), 8.20-8.14 (m, 1H), 7.98 (dd, J=8.3, 3.9 Hz, 1H), 7.93-7.70 (m, 2H), 7.55-7.40 (m, 1H), 7.33-7.31 (m, 1H), 7.26-7.24 (m, 1H), 7.22-7.18 (m, 1H), 6.96 (s, 1H), 6.88 (d, J=9.2 Hz, 1H), 6.15 (d, J=5.5 Hz, 2H), 5.94-5.68 (m, 1H), 5.08 (d, J=3.6 Hz, 1H), 4.82-4.67 (m, 1H), 4.64-4.58 (m, 1H), 4.50 (d, J=9.2 Hz, 1H), 4.42 (t, J=8.2 Hz, 1H), 4.27-4.24 (m, 1H), 4.20-4.15 (m, 4H), 4.10-4.06 (m, 2H), 3.73-3.70 (m, 2H), 3.59-3.48 (m, 2H), 3.28-3.27 (m, 1H), 3.21-3.20 (m, 2H), 3.20-3.19 (m, 2H), 2.38-2.36 (m, 5H), 2.03-1.92 (m, 4H), 1.87-1.83 (m, 1H), 1.79-1.74 (m, 2H), 1.45-1.38 (m, 2H), 1.31-1.21 (m, 4H), 1.16-1.10 (m, 4H), 0.86 (s, 9H).

### Example A57. 2-amino-3-cyclopropyl-N—((S)-6-(6-((17-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-3,6,9,12,15-pentaoxaheptadecyl) carbamoyl)pyridin-3-yl)-2,3-dihydrobenzofuran-3-yl)-N-methyl quinoline-6-carboxamide

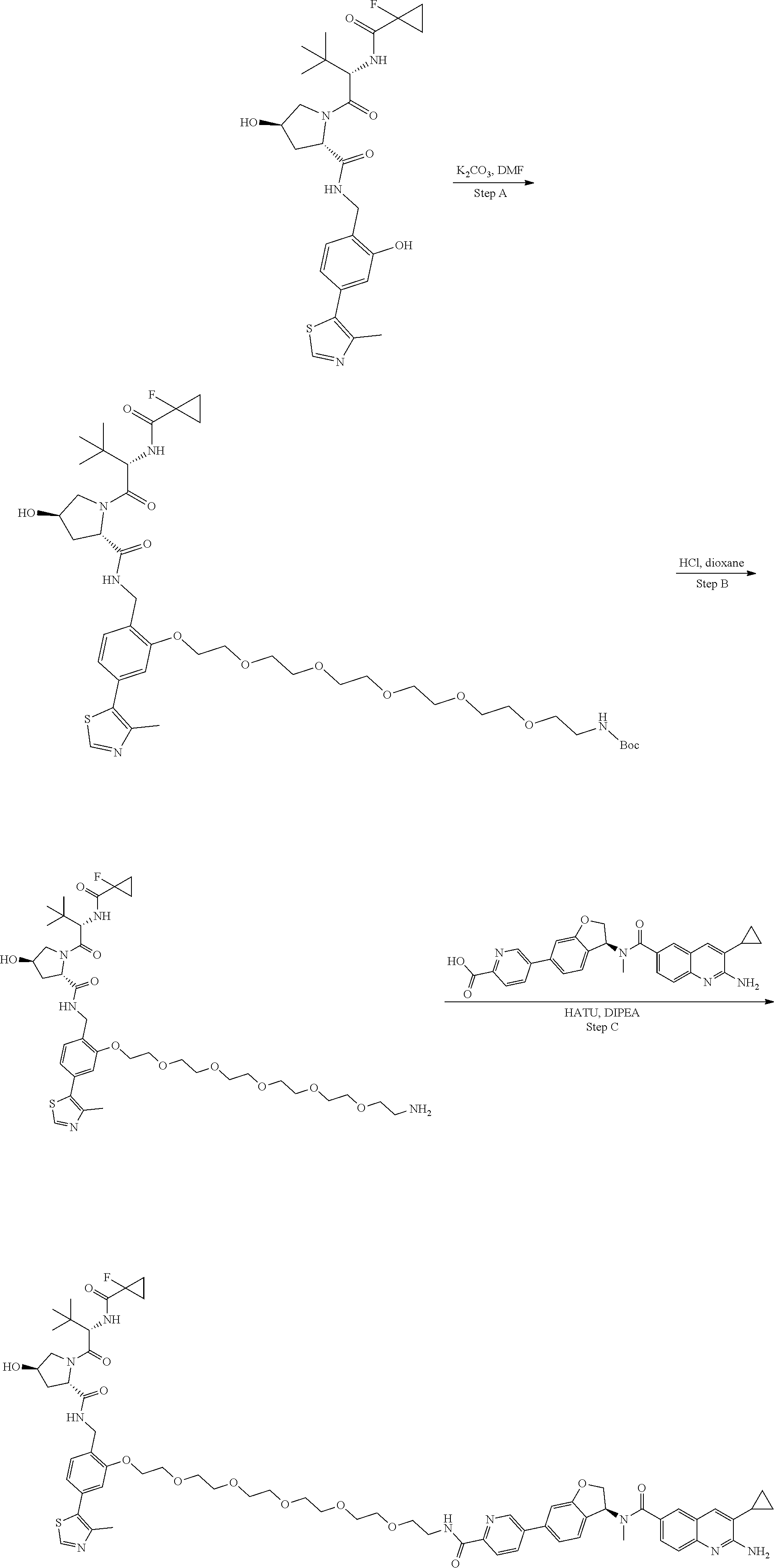

Example A57 was synthesized following the same procedures as described in Example A44 (8 mg, 9% yield over 3 steps) as a white solid. LC-MS (ESI): mass calcd, for C₆₆H₈₀FN₉O₁₃S, 1257.6; m/z found, 1258.9 [M+H]⁺. ¹H NMR (400 MHz, DMSO-d₆) δ 9.06-8.85 (m, 2H), 8.71 (t, J=5.8 Hz, 1H), 8.48 (t, J=5.9 Hz, 1H), 8.27 (dd, J=8.2, 2.2 Hz, 1H), 8.08 (d, J=8.3 Hz, 1H), 7.89-7.69 (m, 2H), 7.64-7.47 (m, 3H), 7.43-7.32 (m, 3H), 7.28 (dd, J=9.2, 2.6 Hz, 1H), 7.03 (d, J=1.4 Hz, 1H), 6.97-6.75 (m, 2H), 4.75 (s, 1H), 4.67 (dd, J=10.2, 3.9 Hz, 1H), 4.59 (d, J=9.1 Hz, 1H), 4.51 (t, J=8.2 Hz, 1H), 4.36-4.26 (m, 2H), 4.23-4.14 (m, 3H), 3.79-3.75 (m, 2H), 3.66-3.59 (m, 4H), 3.57-3.47 (m, 20H), 2.66 (s, 3H), 2.45 (s, 3H), 2.13-2.02 (m, 1H), 1.95-1.80 (m, 2H), 1.40-1.32 (m, 2H), 1.24-1.18 (m, 2H), 1.02-0.97 (m, 2H), 0.95 (s, 9H), 0.68 (d, J=3.8 Hz, 2H).

### Example A58. 2-amino-3-cyclopropyl-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine -2-carboxamido)methyl)phenoxy)ethoxy)cyclohexyl)carbamoyl)pyridin-3-yl)-2,3-dihydrobenzofuran-3-yl)-N-methylquinoline-6-carboxamide

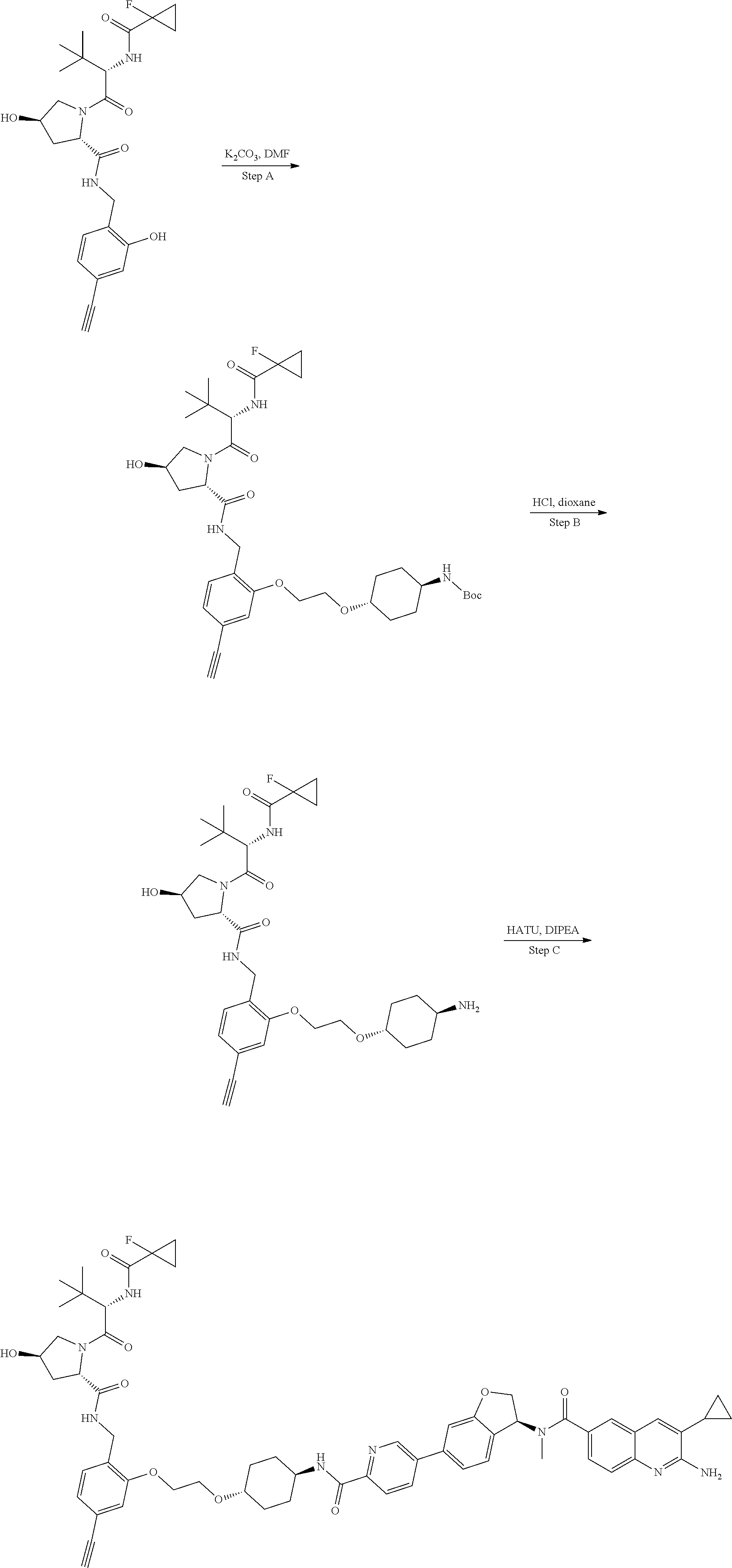 [SMILES: C#Cc1ccc(CNC(=O)[C@@H]2C[C@@H](O)CN2C(=O)[C@@H](NC(=O)C2(F)CC2)C(C)(C)C)c(O)c1 ; C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)OC(C)(C)C)CC2)c1 ; C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1 ; C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CC6)cc5c4)cn3)CC2)c1]

Step A: tert-butyl (15,4r)-4-(2-(5-ethynyl-2-(((2S,4R)-1-(S)-2-(1-fluorocyclopropane-1-carboxamido)-3,3-dimethylbutanoyl)-4-hydroxypyrrolidi-ne-2-carboxamido)methyl) phenoxy)ethoxy)cyclohexyl)carbamate

To a solution of (2S,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide (230 mg, 501 μmol, 1.0 eq.), 2-(((1r,4r)-4-((tert-butoxycarbonyl)amino)cyclohexyl)oxy)ethyl 4-methylbenzenesulfonate (310 mg, 751 μmol, 1.5 eq.) and KI (17 mg, 100 μmol, 0.2 eq.) in DMF (8.0 mL) was added K₂CO₃ (208 mg, 1.50 mmol, 3.0 eq.). The mixture was stirred at 75° C. for 6 hours. The resulting solution was diluted with 50 mL of water and extracted with EtOAc (30 mL×3). The combined organic layers were washed with brine (50 mL), dried over Na₂SO₄ and concentrated under reduced pressure. The residue was purified by flash column chromatography on silica gel (100-200 mesh silica gel, 0-5% MeOH in DCM) to give tert-butyl ((1S,4r)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamate (200 mg, 57% yield) as a yellow oil.

LC-MS (ESI): mass calcd, for C₃₇H₅₃FN₄O₈, 700.38; m/z found, 701.6 [M+H]⁺.

Step B: (2S,4R)—N-(2-(2-(((1r,4S)-4-aminocyclohexyl)oxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide

To a solution of tert-butyl ((1S,4r)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamate (200 mg, 285 μmol, 1.0 eq.) in DCM (6.0 mL) was added HCl-dioxane (2.0 mL) and stirred at room temperature for 1 hour. The resulting solution was concentrated under reduced pressure to give (2S,4R)—N-(2-(2-(((1r,4S)-4-aminocyclohexyl)oxy)ethoxy)-4-ethynylbenzyl)-1-((S)-2-(1-fluorocyclopropane-1-carboxamido)-3,3-dimethylbutanoyl)-4-hydroxypyrrolidi-ne-2-carboxamide (170 mg, 99% yield) as a yellow oil.

LC-MS (ESI): mass calcd, for C₃₂H₄₅FN₄O₆, 600.33; m/z found, 601.1 [M+H]⁺.

### Step C

Example A58 was synthesized following the same procedures as described in Example A29 (5 mg, 14% yield) as a white solid. LC-MS (ESI): mass calcd, for C₆₀H₆₇N₈O₉S, 1062.5; m/z found, 1063.5 [M+H]⁺. ¹H NMR (400 MHz, DMSO-d₆) δ 8.92 (s, 1H), 8.56-8.41 (m, 2H), 8.33-8.20 (m, 1H), 8.08 (d, J=8.2 Hz, 1H), 7.81 (s, 1H), 7.74 (s, 1H), 7.60-7.47 (m, 3H), 7.38 (d, J=7.9 Hz, 1H), 7.35-7.24 (m, 3H), 7.06 (s, 1H), 6.98 (d, J=7.9 Hz, 1H), 6.77 (s, 1H), 4.85-4.64 (m, 2H), 4.59 (d, J=9.2 Hz, 1H), 4.50 (t, J=8.2 Hz, 1H), 4.35 (s, 1H), 4.32-4.17 (m, 2H), 4.15-4.09 (m, 3H), 3.83-3.74 (m, 3H), 3.66-3.58 (m, 2H), 3.47-3.44 (m, 2H), 2.67 (s, 3H), 2.12-2.01 (m, 3H), 1.95-1.81 (m, 4H), 1.54-1.47 (m, 2H), 1.38-1.21 (m, 6H), 1.01-0.92 (m, 11H), 0.71-0.62 (m, 2H).

### Example A59. 2-amino-N—((S)-6-(6-((17-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)-3,6,9,12,15-pentaoxaheptadecyl)carbamoyl)pyridin-3-yl)-2,3-dihydrobenzofuran-3-yl)-N,3-dimethylquinoline-6-carboxamide

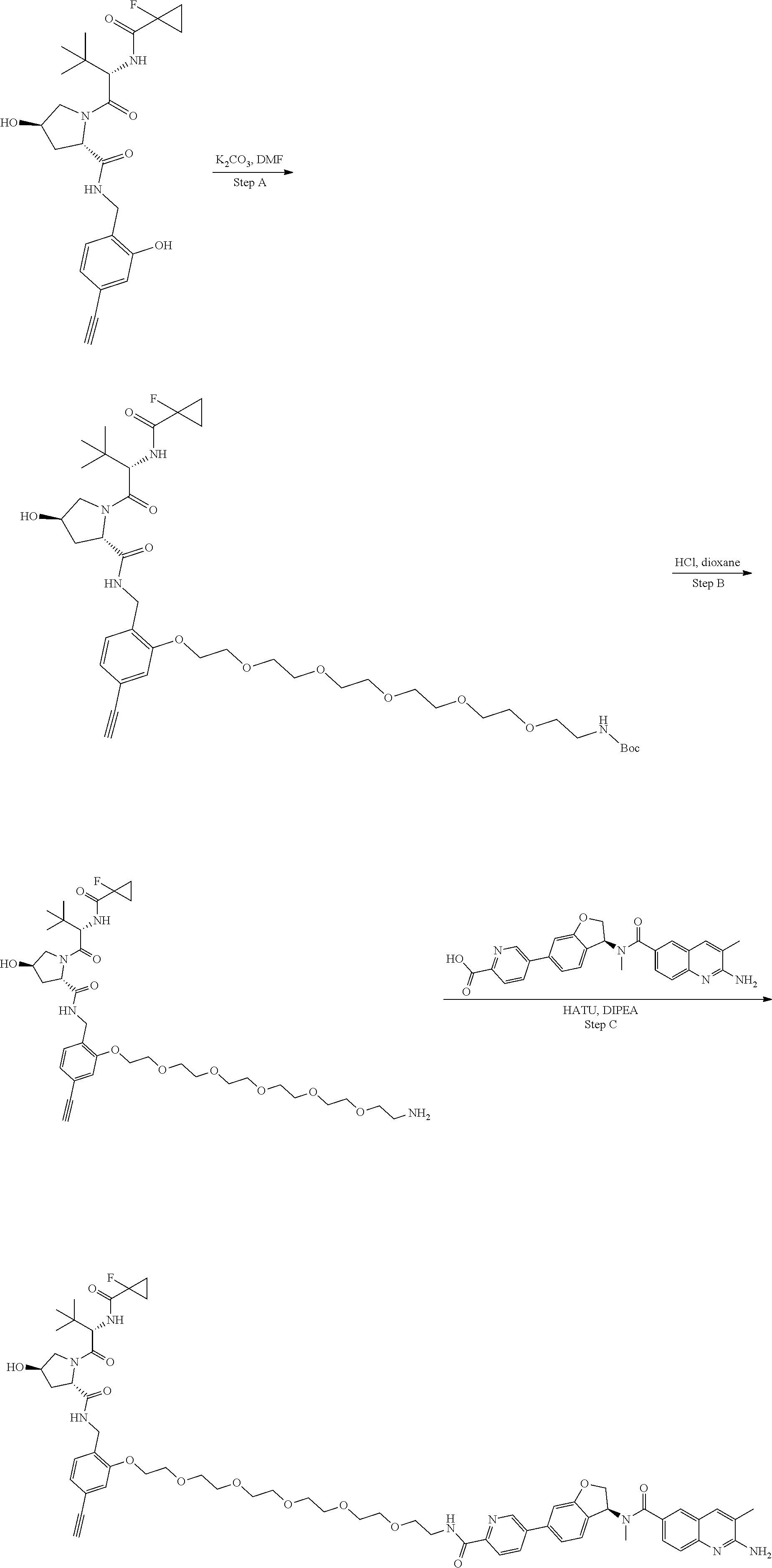

Example A59 was synthesized following the same procedures as described in Example A39 (2.1 mg, 3% yield over 2 steps) as a white solid. LC-MS (ESI): mass calcd, for C₆₂H₇₅FN₈O₁₃, 1158.5; m/z found, 1159.9 [M+H]⁺. ¹H NMR (400 MHz, DMSO-d₆) δ 8.94 (s, 1H), 8.71 (t, J=5.8 Hz, 1H), 8.46 (d, J=6.1 Hz, 1H), 8.28 (d, J=10.1 Hz, 1H), 8.09 (d, J=8.2 Hz, 1H), 7.92 (d, J=47.4 Hz, 2H), 7.73-7.51 (m, 3H), 7.40 (d, J=8.0 Hz, 1H), 7.35 (s, 1H), 7.30 (t, J=8.8 Hz, 2H), 7.03 (s, 1H), 6.96 (d, J=8.0 Hz, 1H), 4.69 (s, 2H), 4.58 (d, J=9.1 Hz, 1H), 4.49 (t, J=8.2 Hz, 1H), 4.34 (s, 1H), 4.29-4.17 (m, 2H), 4.11 (s, 3H), 3.77-3.73 (m, 2H), 3.58 (dd, J=10.2, 5.0 Hz, 6H), 3.54-3.48 (m, 20H), 2.67 (s, 3H), 1.61 (s, 1H), 1.46 (s, 1H), 1.34 (d, J=9.6 Hz, 2H), 1.22 (dd, J=11.3, 3.0 Hz, 2H), 0.95 (s, 9H).

### Example A60. 2-amino-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-carboxamide

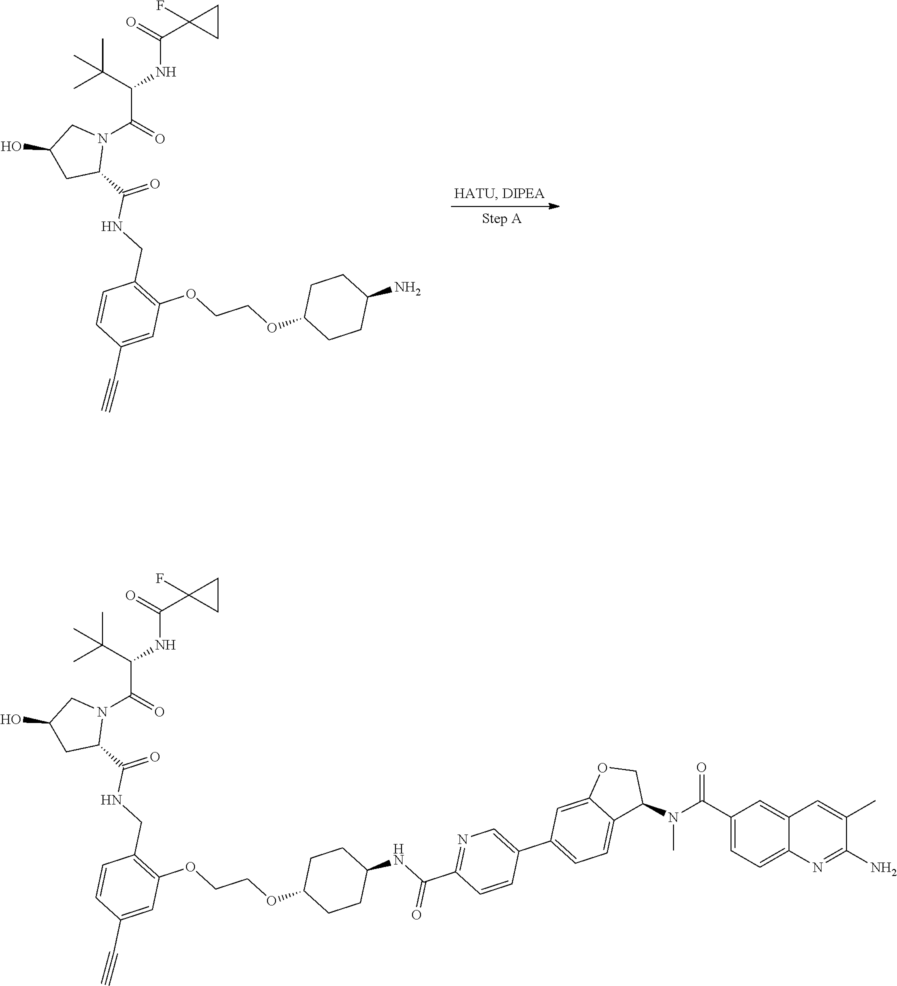 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1 ; C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C)cc5c4)cn3)CC2)c1]

Example A60 was synthesized following the same procedures as described in Example A39 (3.2 mg, 10.2% yield) as a white solid. LC-MS (ESI): mass calcd, for C₆₀H₆₇N₈O₉S, 1036.5; m/z found, 1037.5 [M+H]⁺. ¹H NMR (400 MHz, DMSO-d₆) δ 8.92 (s, 1H), 8.51-8.44 (m, 2H), 8.31-8.24 (m, 1H), 8.08 (d, J=8.4 Hz, 1H), 7.83-7.77 (m, 2H), 7.57-7.53 (m, 2H), 7.52-7.48 (m, 1H), 7.39 (d, J=8.0 Hz, 1H), 7.34-7.27 (m, 3H), 7.06 (s, 1H), 6.98 (d, J=7.5 Hz, 1H), 6.51 (s, 2H), 5.16 (s, 1H), 4.71-4.66 (m, 1H), 4.59 (d, J=9.0 Hz, 1H), 4.52-4.47 (m, 1H), 4.36-4.33 (m, 1H), 4.30-4.25 (m, 1H), 4.23-4.18 (m, 1H), 4.15-4.09 (m, 3H), 3.82-3.76 (m, 3H), 3.66-3.57 (m, 2H), 3.46-3.42 (m, 2H), 2.67 (s, 3H), 2.23 (s, 3H), 2.07-2.00 (m, 3H), 1.88-1.83 (m, 2H), 1.54-1.45 (m, 3H), 1.36-1.30 (m, 3H), 1.24-1.20 (m, 3H), 0.95 (s, 9H).

### Example A61. 4-amino-3-bromo-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,1-dimethyl-1H-pyrazolo[4,3-c]quinoline-8-carboxamide

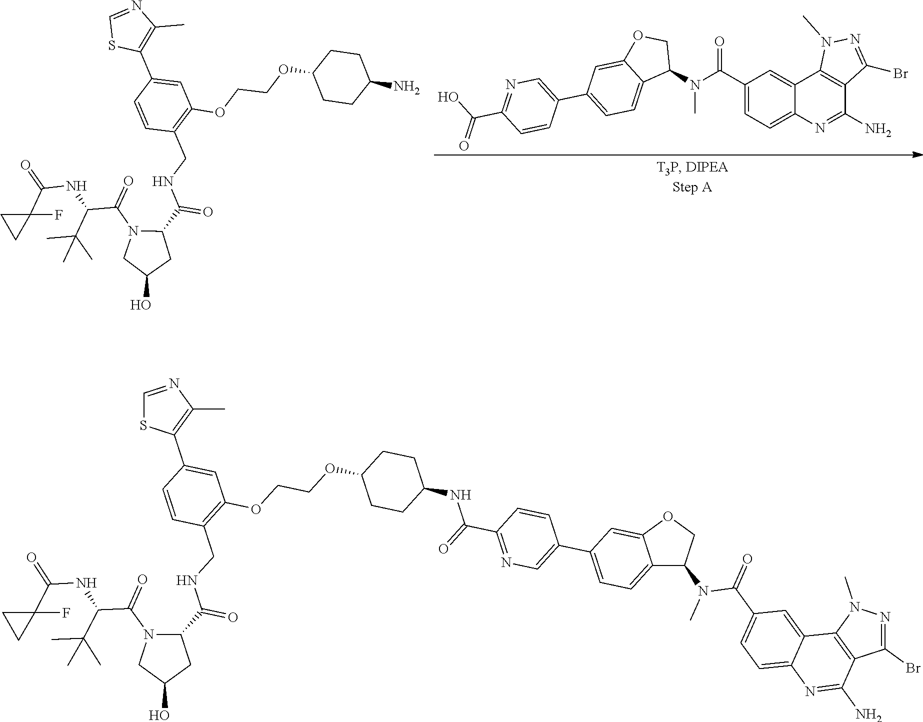 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1 ; CN(C(=O)c1ccc2nc(N)c3c(Br)nn(C)c3c2c1)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c6c(Br)nn(C)c6c5c4)cn3)CC2)c1]

Example A61 was synthesized following the same procedures as described in Example A41 (4.1 mg, 5% yield) as a white solid. LC-MS (ESI): mass calcd, for C₆₁H₆₇BrFN₁₁O₉S, 1227/1229; m/z found, 1227.7/1229.7 [M+H]⁺. ¹H NMR (400 MHz, DMSO-d₆) δ 8.98 (s, 1H), 8.92 (s, 1H), 8.53-8.45 (m, 2H), 8.37 (s, 1H), 8.27 (d, J=7.2 Hz, 1H), 8.08 (d, J=8.1 Hz, 1H), 7.85-7.73 (m, 2H), 7.63-7.57 (m, 1H), 7.43-7.37 (m, 2H), 7.34 (s, 1H), 7.31-7.26 (m, 1H), 7.05 (s, 1H), 6.97 (d, J=7.8 Hz, 1H), 4.84-4.68 (m, 2H), 4.59 (d, J=8.8 Hz, 1H), 4.52 (t, J=7.9 Hz, 1H), 4.45 (s, 3H), 4.37-4.34 (m, 1H), 4.29 (d, J=6.7 Hz, 1H), 4.24 (d, J=5.6 Hz, 1H), 4.19-4.15 (m, 2H), 3.85-3.74 (m, 4H), 3.69-3.54 (m, 5H), 2.70 (s, 3H), 2.46 (s, 3H), 2.13-2.00 (m, 4H), 1.99-1.89 (m, 2H), 1.88-1.82 (m, 2H), 1.54-1.42 (m, 3H), 1.40-1.30 (m, 4H), 0.95 (s, 9H).

### Example A62. N—((S)-6-(6-(((1S,4S)-4-(2-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-2-amino-N,3-dimethylquinoline-6-carboxamide

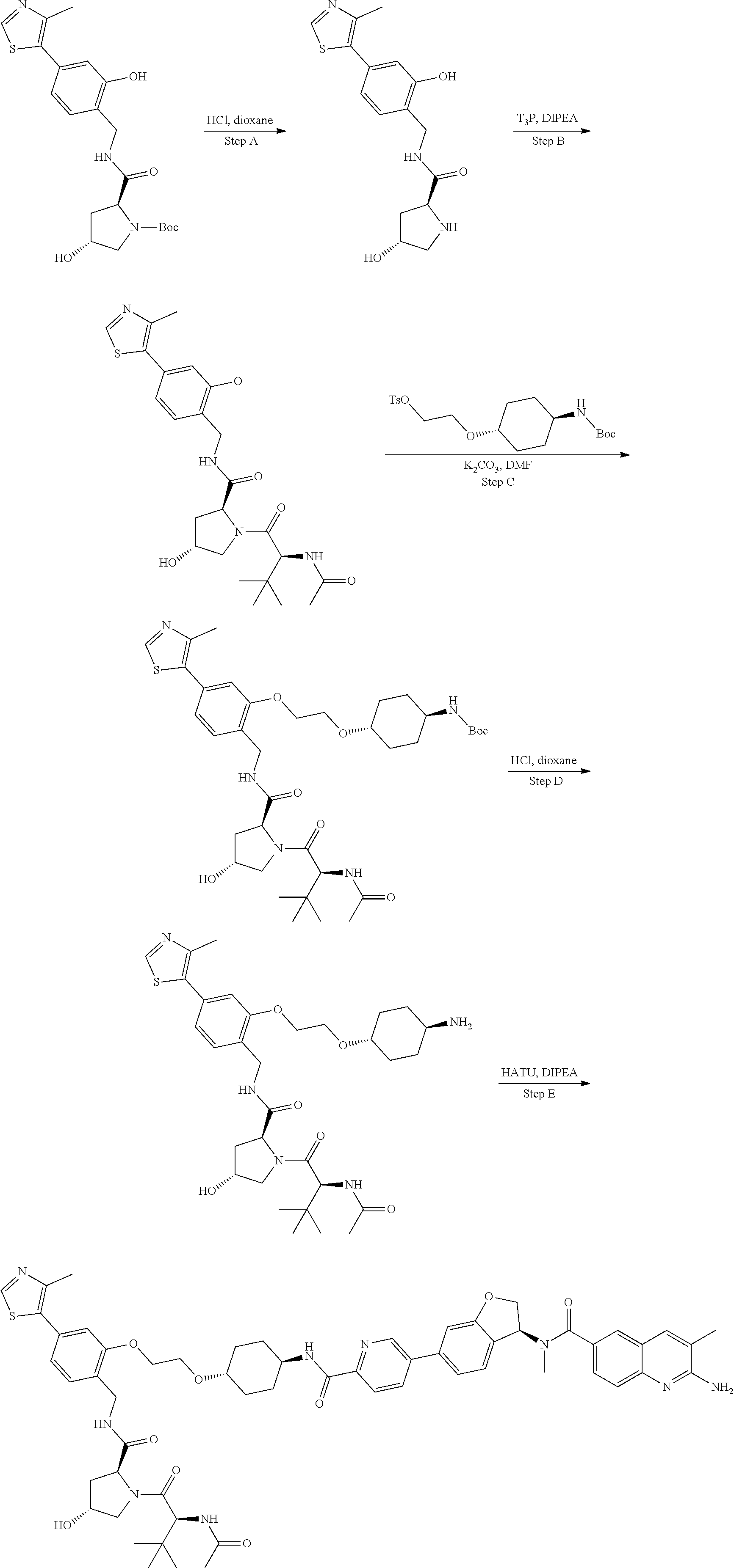

Step A: (2S,4R)-4-hydroxy-N-(2-hydroxy-4-(4-methylthiazol-5-yl)benzyl)pyrrolidine-2-carboxamide

To a solution of tert-butyl(2S,4R)-4-hydroxy-2-((2-hydroxy-4-(4-methylthiazol-5-yl)benzyl)carbamoyl)pyrrolidine-1-carboxylate (170 mg, 392 μmol, 1.0 eq.) in HCl/1,4-Dioxane (2 mL, 4 mol/L) at 0° C. The reaction mixture was stirred for 1 hour at room temperature under N₂ atmosphere. The mixture was concentrated by vacuum to give (2S,4R)-4-hydroxy-N-(2-hydroxy-4-(4-methylthiazol-5-yl)benzyl)pyrrolidine-2-carboxamide (130 mg, 99% yield) as a white solid.

LC-MS (ESI): mass calcd, for C16H19N303S, 333.1; m/z found, 334.2 [M+H]⁺.

Step B: (2S, 4R)-1-((S)-2-acetamido-3,3-dimethylbutanoyl)-4-hydroxy-N-(2-hydroxy-4-(4-methylthiazol-5-yl)benzyl)pyrrolidine-2-carboxamide

To a solution of (2S,4R)-4-hydroxy-N-(2-hydroxy-4-(4-methylthiazol-5-yl)benzyl)pyrrolidine-2-carboxamide (60 mg, 180 μmol, 1.0 eq.), (S)-2-acetamido-3,3-dimethylbutanoic acid (24.9 mg, 144 μmol, 0.8 eq.), T3P (0.08 mL, 270 μmol, 1.5 eq. 50% in DMF) in DMF (1 mL) was added DIEA (69.8 mg, 540 μmol, 3.0 eq.) at room temperature. The reaction mixture was stirred at room temperature for 30 mins under N₂ atmosphere. The mixture was diluted with water (10 mL) and extracted with EtOAc (10 mL×3). The combined organic layers were washed with brine, dried over anhydrous Na₂SO₄ and concentrated under reduced pressure to dryness. The residue was purified by flash chromatographer (eluent, 0~3% MeOH in DCM) to give (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide (50 mg, 57% yield) as yellow oil.

LC-MS (ESI): mass calcd, for C₂₄H₃₂N₄O₅S, 488.2; m/z found, 489.3 [M+H]⁺.

Step C: tert-butyl ((1S,4r)-4-(2-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)ethoxy) cyclohexyl)carbamate

To a mixture of (2S,4R)-1-((S)-2-acetamido-3,3-dimethylbutanoyl)-4-hydroxy-N-(2-hydroxy-4-(4-methylthiazol-5-yl)benzyl)pyrrolidine-2-carboxamide (50 mg, 102 μmol, 1.0 eq.) and 2-(((1r,4r)-4-((tert-butoxycarbonyl)amino)cyclohexyl)oxy)ethyl 4-methylbenzenesulfonate (63.5 mg, 153 μmol, 1.5 eq.) in DMF (2.0 mL) was added K₂CO₃ (42.4 mg, 169 μmol, 3.0 eq.) at room temperature. Then the mixture was stirred at 55° C. for 5 hours. The mixture was diluted with EtOAc, then was washed with H₂O and brine, dried over anhydrous Na₂SO₄, filtered and concentrated under reduced pressure to dryness. The residue was purified by flash chromatographer (eluent, 0~5% MeOH in DCM) to give the tert-butyl ((1S,4r)-4-(2-(2-(((2S,4R)-1-((S)-2-acetamido-3,3-dimethylbutanoyl)-4-hydroxypyrrolidine-2-carboxamido)methyl)-5-(4-methylthiazol-5-yl)phenoxy)ethoxy)cyclohexyl)carbamate (35 mg, 47% yield) as a colorless oil.

LC-MS (ESI): mass calcd, for C₃₇H₅₅N₅O₈S, 729.4; m/z found, 730.3 [M+H]⁺.

### Step D-E

Example A62 was synthesized following the same procedures as described in Example A13 (3.0 mg, 9% yield) as a white solid. LC-MS (ESI): mass calcd, for C₅₈H₆₇N₉O₉S, 1066.3; m/z found, 1067.3 [M+H]⁺. ¹H NMR (400 MHz, DMSO-d₆) δ 8.99 (s, 1H), 8.93 (s, 1H), 8.52-8.47 (m, 2H), 8.31-8.23 (m, 1H), 8.13-8.04 (m, 1H), 8.01-7.91 (m, 1H), 7.85-7.72 (m, 2H), 7.59-7.44 (m, 4H), 7.42-7.36 (m, 1H), 7.34 (s, 1H), 7.05 (s, 1H), 6.98-6.89 (m, 1H), 6.50 (s, 2H), 5.77-5.75 (m, 1H), 4.85-4.64 (m, 2H), 4.56-4.45 (m, 2H), 4.38-4.31 (m, 2H), 4.21-4.14 (m, 3H), 3.83-3.79 (m, 3H), 3.68-3.62 (m, 6H), 2.68 (s, 3H), 2.23 (s, 3H), 2.09-2.01 (m, 4H), 1.92-1.85 (m, 5H), 1.54-1.49 (m, 2H), 1.33-1.28 (m, 2H), 0.93 (s, 9H).

### Example A63. 2-amino-3-bromo-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

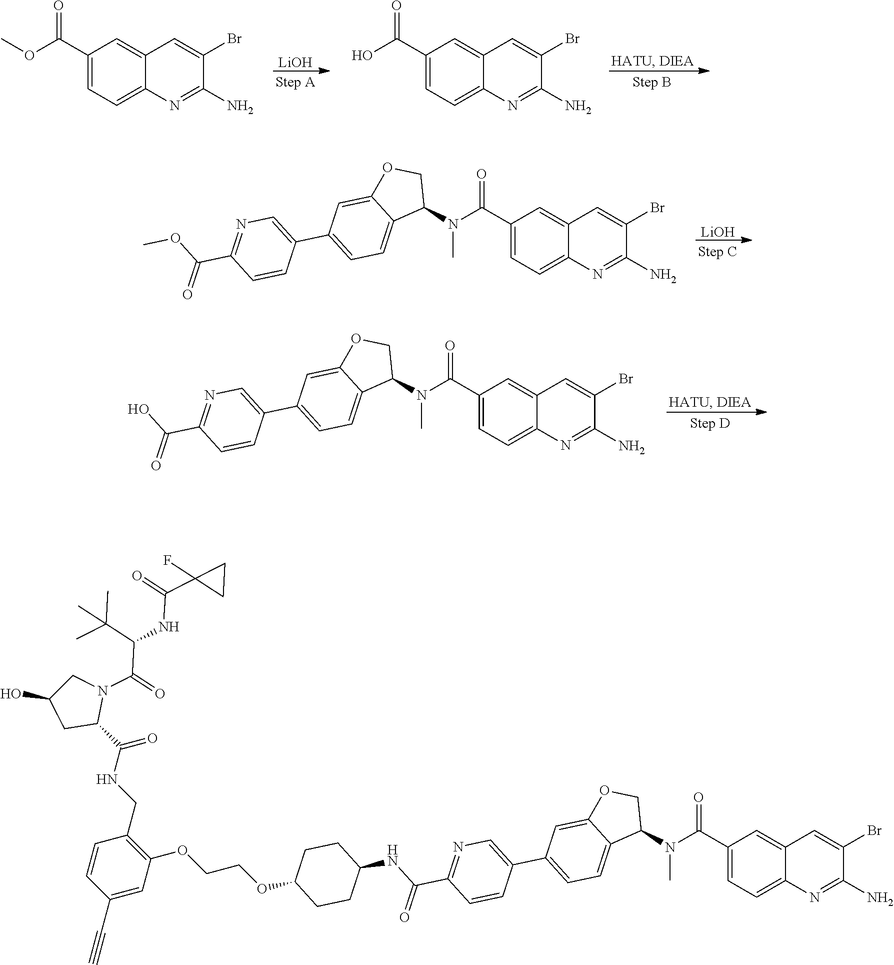

Example A63 was synthesized following the same procedures as described in Example A29 (3.6 mg, 10% yield over 4 steps) as a white solid. LC-MS (ESI): mass calcd. for C₅₇H₆₂BrFN₈O₉, 1100.38; m/z found, 1101.5 [M+H]⁺. ¹H NMR (400 MHz, DMSO-d₆) δ 8.92 (d, J=1.7 Hz, 1H), 8.78-8.61 (m, 1H), 8.54-8.40 (m, 2H), 8.27 (dd, J=8.2, 2.0 Hz, 1H), 8.08 (d, J=8.2 Hz, 1H), 7.98-7.94 (m, 1H), 7.81-7.76 (m, 1H), 7.69-7.63 (m, 1H), 7.55 (d, J=7.8 Hz, 1H), 7.39 (d, J=7.7 Hz, 1H), 7.33 (d, J=6.8 Hz, 1H), 7.30-7.27 (m, 1H), 7.06 (s, 1H), 6.98 (d, J=7.7 Hz, 1H), 4.82-4.67 (m, 2H), 4.59 (d, J=9.4 Hz, 1H), 4.49 (t, J=8.2 Hz, 1H), 4.35 (s, 1H), 4.30 (d, J=6.2 Hz, 1H), 4.26 (d, J=6.2 Hz, 1H), 4.21 (d, J=5.6 Hz, 1H), 4.18-4.15 (m, 1H), 4.14-4.11 (m, 3H), 3.80-3.78 (m, 2H), 3.67-3.65 (m, 2H), 3.39-3.32 (m, 2H), 2.67 (s, 3H), 2.11-1.99 (m, 3H), 1.95-1.88 (m, 1H), 1.88-1.81 (m, 2H), 1.55-1.44 (m, 2H), 1.40-1.26 (m, 4H), 1.23-1.19 (m, 2H), 0.95 (s, 9H).

### Example A64. 2-amino-3-cyclopropyl-N-ethyl-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl) pyridin-3-yl)-2,3-dihydrobenzofuran-3-yl)quinoline-6-carboxamide

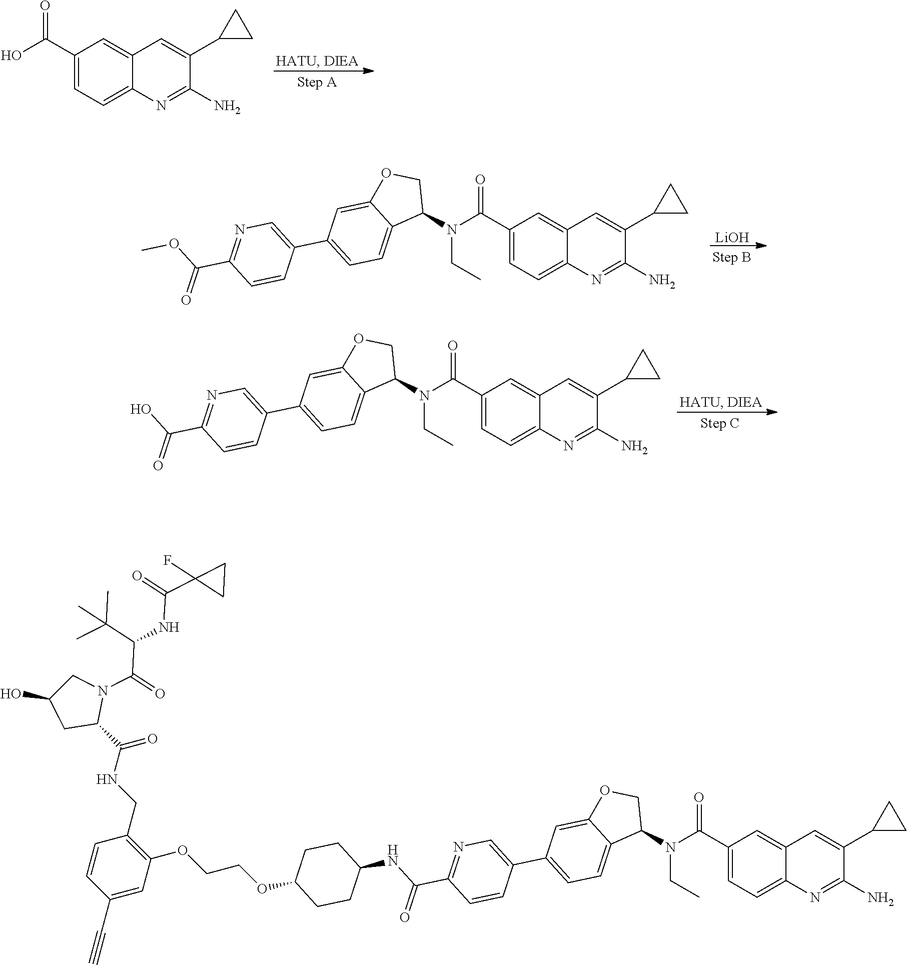

Step A: methyl(S)-5-(3-(2-amino-3-cyclopropyl-N-ethylquinoline-6-carboxamido)-2,3-dihydrobenzofuran-6-yl)picolinate

To a solution of methyl(S)-5-(3-(ethylamino)-2,3-dihydrobenzofuran-6-yl)picolinate (30 mg, 101 μmol, 1 eq.) in DMF (3 mL) was added 2-amino-3-cyclopropylquinoline-6-carboxylic acid (25 mg, 111 μmol, 1.1 eq.), HATU (77 mg, 201 μmol, 2 eq.) and DIEA (52 mg, 402 μmol, 4 eq.) at 0° C. The mixture was then stirred at room temperature for 1 hour under N₂ atmosphere. The resulting solution was diluted with 30 mL of H₂O and extracted with EtOAc (30 mL×3). The combined organic layers were washed with brine, dried over Na₂SO₄, and concentrated under reduced pressure. The residue was purified by flash column chromatography on silica gel (100-200 mesh silica gel, 0-7% MeOH in DCM) to afford methyl(S)-5-(3-(2-amino-3-cyclopropyl-N-ethylquinoline-6-carboxamido)-2,3-dihydrobenzofuran-6-yl)picolinate (27 mg, 53% yield) as a yellow oil.

LC-MS (ESI): mass calcd, for C₃₀H₂₈N₄O₄, 508.2; m/z found, 509.2 [M+H]⁺.

### Step B—C

Example A64 was synthesized following the same procedures as described in Example A50 (6.7 mg, 12% yield over 2 steps) as a white solid. LC-MS (ESI): mass calcd. for C₆₁H₆₉FN₈O₉, 1076.5; m/z found, 1077.3 [M+H]⁺. ¹H NMR (400 MHz, DMSO-d₆) δ 8.92 (d, J=1.9 Hz, 1H), 8.76 (s, 2H), 8.53-8.41 (m, 2H), 8.32-8.17 (m, 2H), 8.08 (d, J=8.2 Hz, 1H), 8.00 (s, 1H), 7.88-7.71 (m, 2H), 7.57 (d, J=7.8 Hz, 1H), 7.43-7.24 (m, 4H), 7.06 (s, 1H), 6.98 (d, J=8.5 Hz, 1H), 4.67-4.62 (m, 1H), 4.59 (d, J=9.2 Hz, 1H), 4.53-4.47 (m, 1H), 4.35 (s, 1H), 4.31-4.19 (m, 2H), 4.15-4.10 (m, 3H), 3.81-3.76 (m, 3H), 3.67-3.62 (m, 5H), 3.39-3.31 (m, 2H), 2.12-2.00 (m, 3H), 1.95-1.80 (m, 4H), 1.57-1.46 (m, 2H), 1.45-1.28 (m, 4H), 1.27-1.14 (m, 3H), 1.10-1.05 (m, 2H), 1.00-0.88 (m, 11H), 0.83-0.79 (m, 2H).

### Example A65. N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzo furan-3-yl)-N,3-dimethylquinoline-6-carboxamide

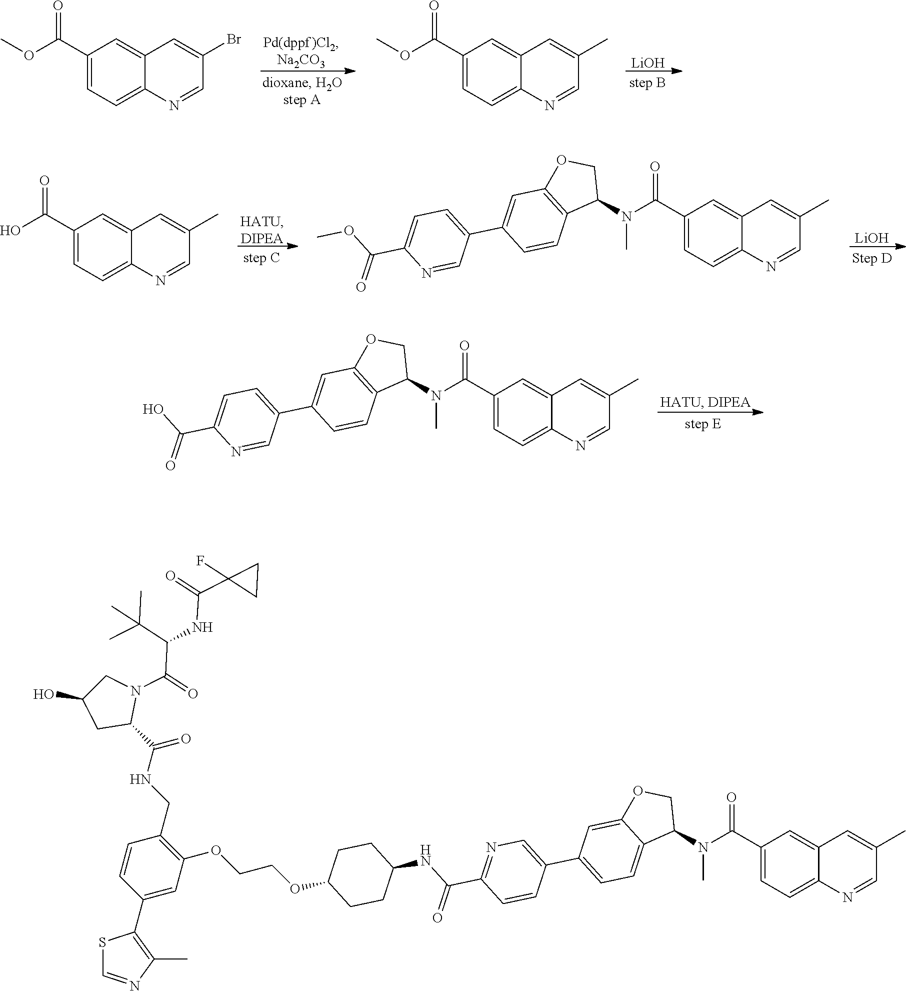

Step A: methyl 3-methylquinoline-6-carboxylate

To a mixture of methyl 3-bromoquinoline-6-carboxylate (1.0 g, 3.8 mmol, 1.0 eq.), 2,4,6-Trimethyl-1,3,5,2,4,6-trioxatriborinane (4.2 mL, 50% wt, 15.0 mmol, 4.0 eq.), Xphos Pd G2 (296 mg, 376 μmol, 0.1 eq.) and K₃PO₄ (1.6 g, 7.5 mmol, 2.0 eq.) in 1,4-dioxane (20 mL) was stirred at 100° C. under N₂ overnight. The mixture was poured into EtOAc (80 mL) and filtered. The filter cake was washed with EtOAc (20 mL×3). The combined filtrates were washed with brine, dried over Na₂SO₄ and concentrated under reduced pressure. The residue was purified by flash column chromatography on silica gel (100-200 mesh silica gel, 0-15% EtOAc in PE) to give methyl 3-methylquinoline-6-carboxylate (730 mg, 92% yield) as a yellow solid.

LC-MS (ESI): mass calcd, for C₁₂H₁₁NO₂, 201.2; m/z found, 202 [M+H]⁺.

Step B: lithium 3-methylquinoline-6-carboxylic

To a solution of methyl 3-methylquinoline-6-carboxylate (330 mg, 1.6 mmol, 1.0 eq.) in THF (5.0 mL) and Water (5.0 mL) was added LiOH (138 mg, 3.3 mmol, 2.0 eq.). The mixture was stirred at room temperature for 2 hours. The resulting solution was concentrated under reduced pressure to give lithium 3-methylquinoline-6-carboxylic (300 mg, 97% yield) as a yellow solid.

LC-MS (ESI): mass calcd, for C₁₁H₉NO₂, 187.2; m/z found, 188.3 [M+H]⁺.

Step C: methyl(S)-5-(3-(N,3-dimethylquinoline-6-carboxamido)-2,3-dihydrobenzofuran-6-yl)picolinate

To a solution of lithium 3-methylquinoline-6-carboxylic (50 mg, 267 μmol, 1.0 eq.) in DMF (4 mL) was added methyl 5-(3-(methylamino)-2,3-dihydrobenzofuran-6-yl)picolinate (84 mg, 294 μmol, 1.1 eq.), HATU (152 mg, 401 μmol, 1.5 eq.) and DIEA (140 μL, 801 μmol, 3.0 eq.). The mixture was stirred at room temperature for 1 hour. The resulting solution was diluted with 10 mL of water and extracted with EtOAc (10 mL×3). The combined organic layers were washed with brine, dried over Na₂SO₄ and concentrated under reduced pressure. The residue was purified by flash column chromatography on silica gel (100-200 mesh silica gel, 0-60% EtOAc in PE) to give methyl(S)-5-(3-(N,3-dimethylquinoline-6-carboxamido)-2,3-dihydrobenzofuran-6-yl)picolinate (80 mg, 66% yield) as a yellow solid.

LC-MS (ESI): mass calcd, for C₂₇H₂₃N₃O₄, 453.1; m/z found, 454.4 [M+H]⁺.

### Step D-E

Example A65 was synthesized following the same procedures as described in Example A13 (16 mg, 40% yield over 2 steps) as a white solid. LC-MS (ESI): mass calcd. for C₆₀H₆₇FN₈O₉S, 1095.3; m/z found, 1096.7 [M+H]⁺. ¹H NMR (400 MHz, DMSO-d₆) δ 9.00 (s, 1H), 8.93 (s, 1H), 8.88 (s, 1H), 8.51-8.47 (m, 2H), 8.30-8.26 (m, 2H), 8.12-8.09 (m, 2H), 8.08-8.06 (m, 1H), 7.82 (d, J=7.2 Hz, 1H), 7.60 (d, J=7.4 Hz, 1H), 7.43-7.39 (m, 2H), 7.35 (s, 1H), 7.30 (d, J=6.7 Hz, 1H), 7.06 (s, 1H), 6.98 (d, J=7.8 Hz, 1H), 6.45 (s, 1H), 5.63 (s, 1H), 4.84 (s, 1H), 4.75-4.71 (m, 1H), 4.60 (d, J=9.2 Hz, 1H), 4.53 (t, J=8.1 Hz, 1H), 4.37 (s, 1H), 4.32-4.23 (m, 2H), 4.20-4.17 (m, 2H), 3.83-3.79 (m, 3H), 3.67-3.61 (m, 2H), 3.51-3.46 (m, 2H), 2.72-2.65 (m, 3H), 2.53 (s, 3H), 2.47 (s, 3H), 2.14-2.09 (m, 1H), 2.08-2.04 (m, 2H), 1.92-1.89 (m, 2H), 1.71-1.68 (m, 1H), 1.55-1.48 (m, 2H), 1.37-1.33 (m, 2H), 1.24-1.21 (m, 2H), 0.96 (s, 9H)

### Example A66, N1-(7-amino-1-ethyl-1H-pyrazolo[3,4-c]pyridin-4-yl)-N2-ethyl-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)oxalamide

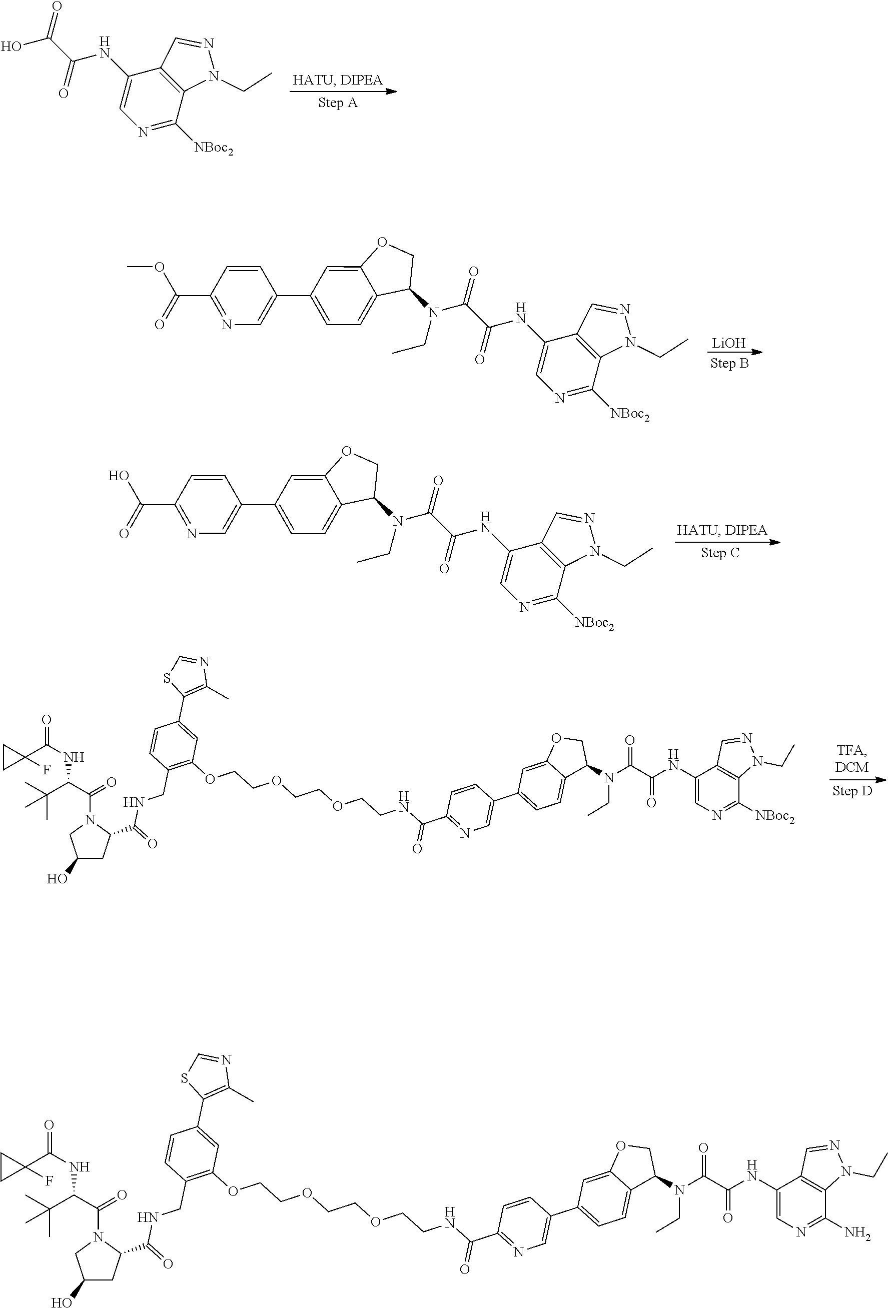

Example A66 was synthesized following the same procedures as described in Example A41 (4.3 mg, 13% yield over 4 steps) as a white solid. LC-MS (ESI): mass calcd. for C₅₈H₆₉FN₁₂O₁₁S, 1161.3; m/z found, 1162.4 [M+H]⁺. ¹H NMR (400 MHz, DMSO-d₆) δ 10.76 (d, J=41.5 Hz, 1H), 8.94 (d, J=4.2 Hz, 1H), 8.92-8.87 (m, 1H), 8.71 (t, J=4.7 Hz, 1H), 8.49 (t, J=5.8 Hz, 1H), 8.28-8.22 (m, 1H), 8.09-7.82 (m, 3H), 7.63-7.47 (m, 1H), 7.41-7.35 (m, 2H), 7.34-7.28 (m, 2H), 6.99 (s, 1H), 6.94 (d, J=7.7 Hz, 1H), 6.25 (s, 2H), 6.02-5.73 (m, 1H), 5.22 (d, J=3.5 Hz, 1H), 4.84-4.74 (m, 1H), 4.67-4.57 (m, 4H), 4.50 (t, J=8.3 Hz, 1H), 4.35 (s, 1H), 4.30-4.21 (m, 2H), 4.17-4.15 (m, 2H), 3.82-3.78 (m, 4H), 3.64-3.63 (m, 3H), 3.39-3.36 (m, 3H), 3.19-3.12 (m, 2H), 2.51 (s, 3H), 2.43-2.41 (m, 3H), 2.11-2.05 (m, 1H), 1.94-1.88 (m, 1H), 1.36-1.33 (m, 3H), 1.23-1.19 (m, 2H), 1.03 (t, J=7.1 Hz, 2H), 0.94 (s, 9H).

### Example A67. 2-amino-N-(cyclopropylmethyl)-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl) pyridin-3-yl)-2,3-dihydrobenzofuran-3-yl)-3-methylquino-line-6-carboxamide

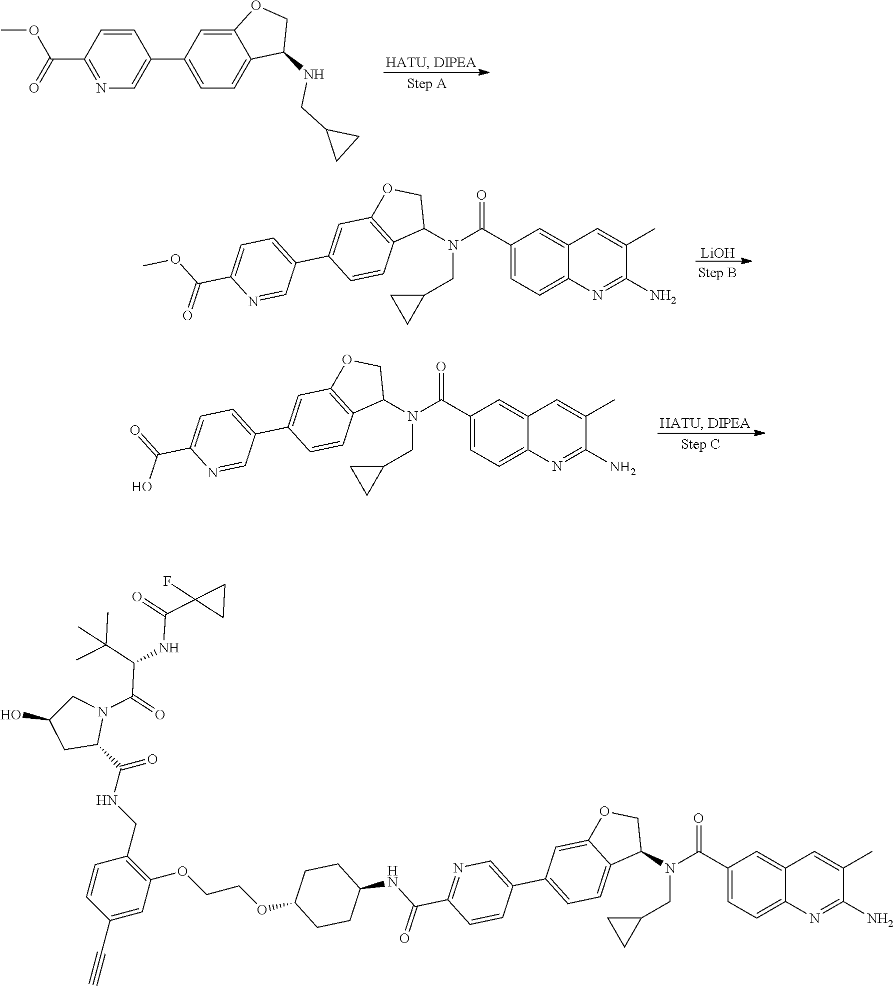

Example A67 was synthesized following the same procedures as described in Example A51 (2.0 mg, 6.0% yield over 3 steps) as a white solid. LC-MS (ESI): mass calcd. For C₆₁H₆₉N₈O₉, 1076.5; m/z found, 1077.3 [M+H]⁺. ¹H NMR (400 MHz, DMSO-d₆) δ 8.51-8.42 (m, 2H), 8.30-8.04 (m, 1H), 7.78 (d, J=30.3 Hz, 1H), 7.61-7.48 (m, 2H), 7.37 (d, J=8.7 Hz, 1H), 7.34-7.24 (m, 3H), 7.22-7.17 (m, 1H), 7.07-7.03 (m, 1H), 6.97 (d, J=6.9 Hz, 1H), 6.65 (s, 1H), 6.49 (s, 1H), 5.33 (d, J=4.6 Hz, 1H), 5.17 (d, J=3.6 Hz, 1H), 4.88-4.67 (m, 1H), 4.59 (d, J=9.4 Hz, 1H), 4.53-4.43 (m, 1H), 4.35 (s, 1H), 4.31-4.17 (m, 2H), 4.16-4.07 (m, 3H), 3.81-3.73 (m, 2H), 3.71-3.50 (m, 2H), 3.43-3.38 (m, 2H), 3.26-3.21 (m, 2H), 2.22 (s, 3H), 2.02-1.99 (m, 3H), 1.89-1.82 (m, 2H), 1.79-1.70 (m, 2H), 1.60-1.54 (m, 2H), 1.48-1.45 (m, 2H), 1.39-1.37 (m, 2H), 1.14 (d, J=6.7 Hz, 2H), 0.95 (s, 9H), 0.88-0.85 (m, 4H).

### Example A68. 2-amino-3-bromo-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-isopropylquinoline-6-carboxamide

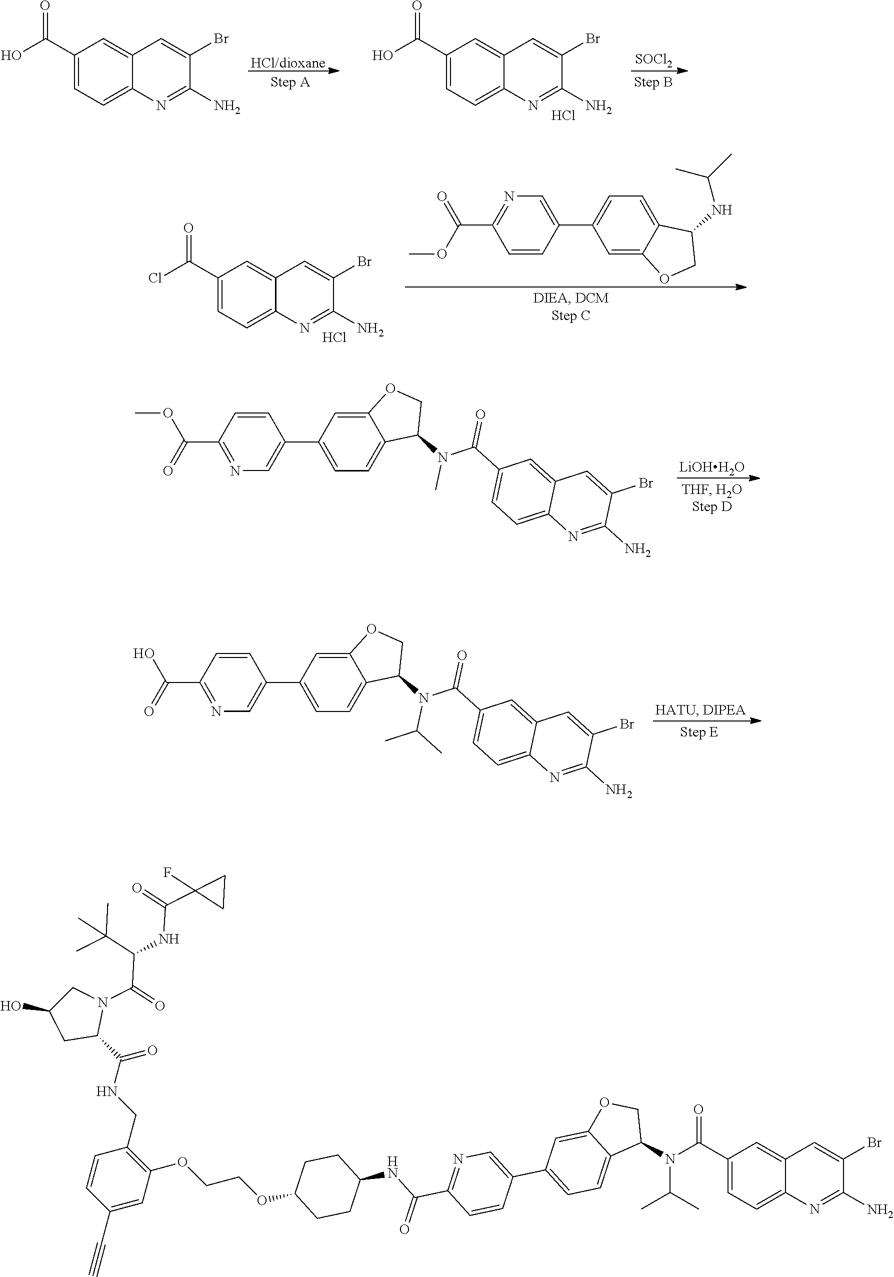

Step A: 2-amino-3-bromoquinoline-6-carboxylic acid hydrochloride

To a solution of 2-amino-3-bromoquinoline-6-carboxylic acid (100 mg, 376 mmol, 1.0 eq.) in Dioxane (1 mL) was added Hydrogen chloride (1 mL, 4 M/L), the reaction mixture was stirred at room temperature for 0.5 hour. The reaction mixture was concentrated under reduced pressure to afford 2-amino-3-bromoquinoline-6-carboxylic acid hydrochloride (113 mg, 98% yield) as a white solid.

Step B: 2-amino-3-bromoquinoline-6-carbonyl chloride

To a stirred mixture of 2-amino-3-bromoquinoline-6-carboxylic acid (100 mg, 374 μmol, 1. 0 eq.) in SOCl₂ (2 mL) were added Pyridine (3.03 μL, 37.4 μmol, 0.1 eq.). The resulting mixture was stirred at 55° C. for 1 hour. The reaction mixture was concentrated to afford 2-amino-3-bromoquinoline-6-carbonyl chloride (100 mg, 94% yield) as a white solid.

Step C: methyl(S)-5-(3-(2-amino-3-bromo-N-isopropylquinoline-6-carboxamido)-2,3-dihydrobenzofuran-6-yl)picolinate

To a stirred mixture of methyl(S)-5-(3-(isopropylamino)-2,3-dihydrobenzofuran-6-yl)picolinate (30.0 mg, 96.0 μmol, 1.0 eq.) in DCM (2 mL) were added DIEA (62 mg, 83.6 μL, 480 μmol, 5.0 eq.) and 2-amino-3-bromoquinoline-6-carbonyl chloride (82 mg, 288 μmol, 3.0 eq.) slowly at 0° C. The resulting mixture was stirred at room temperature for 1 hour. The reaction mixture was poured into water and then extracted with DCM (50 mL×2). The combined organic layers were washed with brine, dried over anhydrous Na₂SO₄ and concentrated. The residue was purified by column chromatography (silica gel, 0% EA in PE ~ 10% methanol in dichloromethane) and prep-HPLC(TFA 0.1%) to afford methyl(S)-5-(3-(2-amino-3-bromo-N-isopropylquinoline-6-carboxamido)-2,3-dihydrobenzofuran-6-yl)picolinate (29 mg, 54% yield) as a white solid.

LC-MS (ESI): mass calcd. For C₂₈H₂₅BrN₄O₄, 560.11; m/z found, 563. 1 [M+H]⁺.

### Step D-E

Example A68 was synthesized following the same procedures as described in Example A45 (9.2 mg, 12% yield over 2 steps) as a white solid. LC-MS (ESI): mass calcd. For C₅₉H₆₆BrFN₈O₉, 1128.41; m/z found, 1129.8 [M+H]⁺. ¹H NMR (400 MHz, DMSO-d₆) δ 8.89 (s, 1H), 8.47 (s, 3H), 8.23 (d, J=8.3 Hz, 1H), 8.07 (d, J=8.1 Hz, 1H), 7.71 (s, 1H), 7.57-7.46 (m, 2H), 7.37 (s, 1H), 7.34-7.24 (m, 3H), 7.19 (s, 1H), 7.06 (s, 1H), 6.97 (d, J=7.6 Hz, 1H), 6.90-6.76 (m, 2H), 5.41 (s, 1H), 5.16 (s, 1H), 4.75 (t, J=8.7 Hz, 1H), 4.70-4.65 (m, 1H), 4.59 (d, J=8.8 Hz, 1H), 4.49 (t, J=8.1 Hz, 1H), 4.35 (s, 1H), 4.30-4.25 (m, 1H), 4.20 (d, J=5.5 Hz, 1H), 4.15-4.08 (m, 3H), 3.85-3.73 (m, 4H), 3.64-3.59 (m, 2H), 2.08-2.02 (m, 2H), 1.88-1.81 (m, 2H), 1.55-1.47 (m, 2H), 1.41-1.38 (m, 2H), 1.35-1.31 (m, 2H), 1.26-1.19 (m, 4H), 1.06-0.86 (m, 15H).

### Example A69. N-(cyclopropylmethyl)-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-3-methylquino-line-6-carboxamide

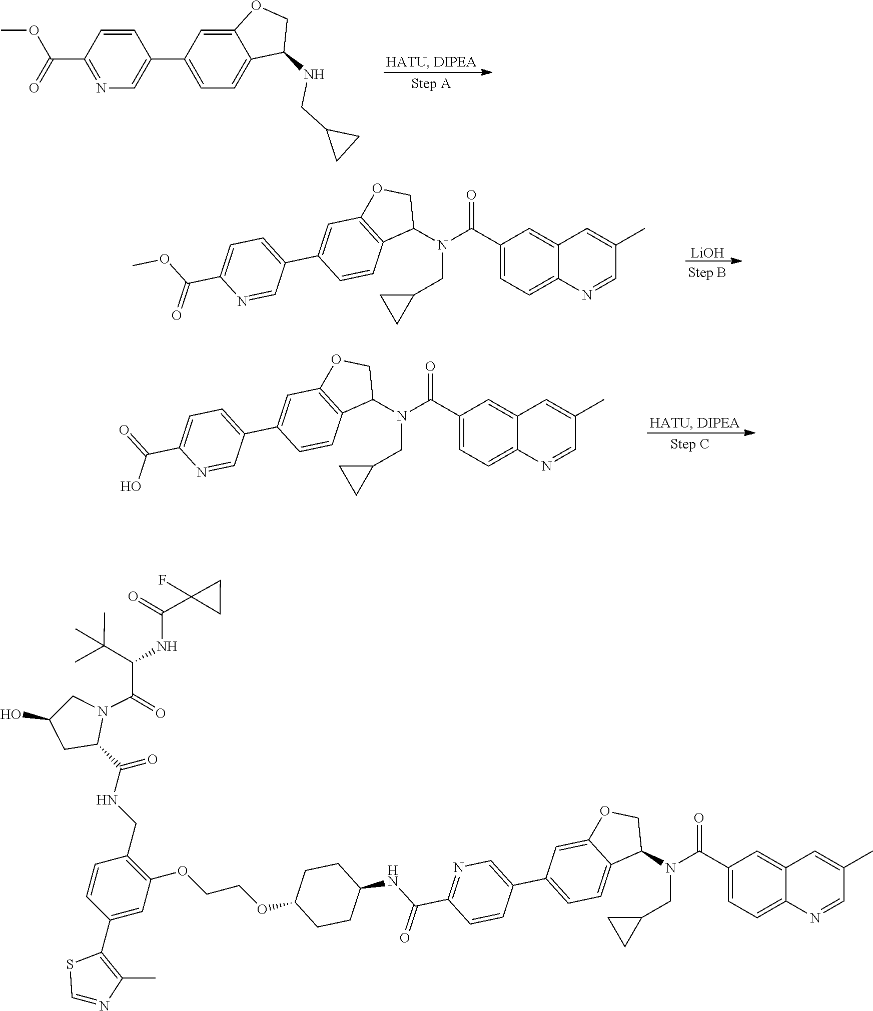

Step A: methyl 5-(3-(N-(cyclopropylmethyl)-3-methylquinoline-6-carboxamido)-2,3-dihydrobenzofuran-6-yl)picolinate

A solution of 3-methylquinoline-6-carboxylic acid (15 mg, 80 μmol, 1.0 eq.), methyl 5-(3-((cyclopropylmethyl)amino)-2,3-dihydrobenzofuran-6-yl)picolinate (29 mg, 88 μmol, 1.1 eq) and HATU (46 mg, 120 μmol, 1.5 eq.) in DMF (2.0 mL) was added DIEA (42 μL, 240 μmol, 3.0 eq.) and stirred for 1 hour at room temperature. The resulting solution was diluted with 30 mL of water and extracted with EtOAc (10 mL×3). The combined organic layers were washed with brine (30 mL), dried over Na₂SO₄ and concentrated under reduced pressure. The residue was purified by flash column chromatography on silica gel (100-200 mesh silica gel, 0-60% EtOAc in PE) to give methyl 5-(3-(N-(cyclopropylmethyl)-3-methylquinoline-6-carboxamido)-2,3-dihydrobenzofuran-6-yl)picolinate (20 mg, 51% yield) as a yellow solid.

LC-MS (ESI): mass calcd, for C₃₀H₂₇N₃O₄, 493.6; m/z found, 494.5 [M+H]⁺.

Step B: 5-(3-(N-(cyclopropylmethyl)-3-methylquinoline-6-carboxamido)-2,3-dihydrobenzofuran-6-yl)picolinic acid

A solution of methyl 5-(3-(N-(cyclopropylmethyl)-3-methylquinoline-6-carboxamido)-2,3-dihydrobenzofuran-6-yl)picolinate (20 mg, 41 μmol, 1.0 eq.) in THF (2.0 mL) and Water (2.0 mL) was added Lithium hydroxide monohydrate (4 mg, 81 μmol, 2.0 eq.) and stirred at room temperature for 1 hour. The resulting solution was concentrated under reduced pressure to give 5-(3-(N-(cyclopropylmethyl)-3-methylquinoline-6-carboxamido)-2,3-dihydrobenzofuran-6-yl) picolinic acid (12 mg, 60% yield) as a yellow solid.

LC-MS (ESI): mass calcd, for C₂₉H₂₅N₃O₄, 479.5; m/z found, 480.4 [M+H]⁺.

Step C: N-(cyclopropylmethyl)-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-3-methylquinoline-6-carboxamide

To a solution of 5-(3-(N-(cyclopropylmethyl)-3-methylquinoline-6-carboxamido)-2,3-dihydrobenzofuran-6-yl)picolinic acid (15 mg, 31 μmol, 1.0 eq.), (2S,4R)—N-(2-(2-(((1r,4S)-4-aminocyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (23 mg, 34 μmol, 1.1 eq.) and HATU (18 mg, 47 μmol, 1.5 eq.) in DMF (2.0 mL) was added DIPEA (16 μL, 94 μmol, 3.0 eq.). The mixture was stirred at room temperature for 1 hour. The resulting solution was diluted with 10 mL of water and extracted with EtOAc (10 mL×3). The combined organic layers were washed with brine (20 mL), dried over Na₂SO₄ and concentrated under reduced pressure. The residue was purified by prep-HPLC to give tert-butyl 4-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-1,2,3,6-tetrahydropyridine-1-carbonyl) piperidine-1-carboxylate (5.8 mg, 16% yield) as a white solid.

LC-MS (ESI): mass calcd, for C₆₃H₇₁FN₈O₉S, 1134.5; m/z found, 1135.6 [M+H]⁺. 1H NMR (400 MHz, DMSO-d₆) δ 9.20 (s, 1H), 9.15-9.08 (m, 2H), 8.74-8.66 (m, 2H), 8.54-8.45 (m, 2H), 8.32-8.26 (m, 3H), 8.01 (d, J=8.6 Hz, 1H), 7.83 (d, J=7.8 Hz, 1H), 7.63-7.57 (m, 2H), 7.55-7.46 (m, 2H), 7.26 (d, J=1.2 Hz, 1H), 7.18 (dd, J=7.8, 1.2 Hz, 1H), 4.97 (d, J=5.6 Hz, 2H), 4.80 (d, J=9.2 Hz, 1H), 4.73 (t, J=8.2 Hz, 1H), 4.57 (s, 1H), 4.54-4.41 (m, 2H), 4.40-4.35 (m, 2H), 4.05-3.98 (m, 3H), 3.89-3.81 (m, 2H), 3.70-3.68 (m, 1H), 3.41-3.26 (m, 3H), 2.73 (s, 3H), 2.68 (s, 3H), 2.32-2.22 (m, 3H), 2.18-2.12 (m, 1H), 2.07 (d, J=10.2 Hz, 2H), 1.77-1.68 (m, 2H), 1.60-1.49 (m, 4H), 1.48-1.39 (m, 4H), 1.21-1.10 (m, 10H), 0.62-0.46 (m, 2H).

### Example A70. 2-amino-3-cyclopropyl-N—((S)-6-(6-((2-(2-(2-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl) carbamoyl)pyridin-3-yl)-2,3-dihydrobenzofuran-3-yl)-N-methylquino-line-6-carboxamide

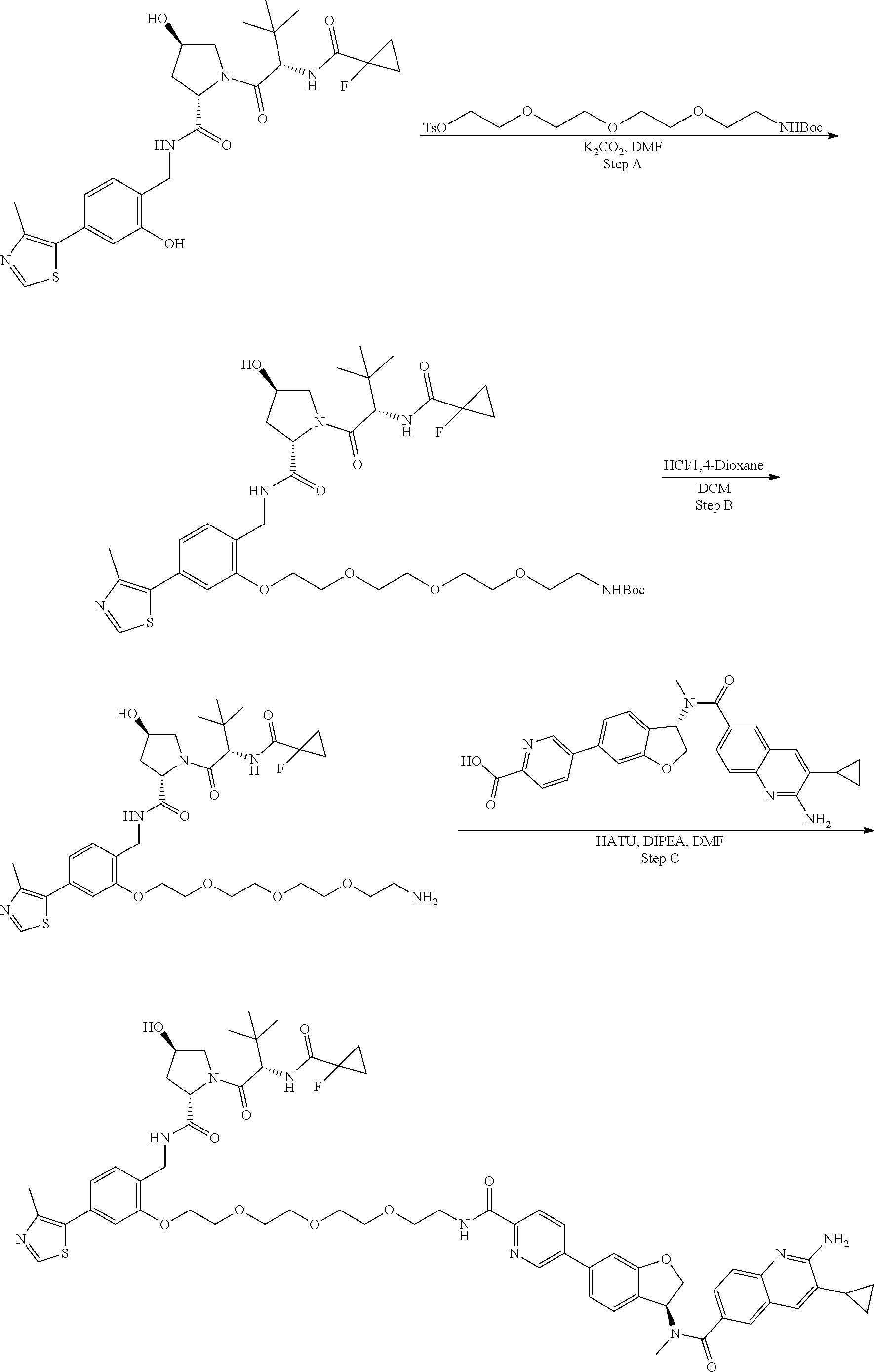

Step A: tert-butyl(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)carbamate

To a mixture of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl) pyrrolidi ne-2-carboxamide (30 mg, 56 μmol, 1.0 eq.) and 2,2-dimethyl-4-oxo-3,8,11,14-tetraoxa-5-azahexadecan-16-yl 4-methylbenzenesulfonate (33 mg, 73 μmol, 1.3 eq.) in DMF (3.00 mL) was added K₂CO₃ (31 mg, 225 μmol, 4.0 eq.). The mixture was stirred at 55° C. for 16 hours under N₂ atmosphere. The resulting solution was diluted with 100 mL of water and extracted with EtOAc (100 mL×3). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford tert-butyl(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)carbamate (30 mg, 66% yield) as a white solid. LC-MS (ESI): mass calcd, for C₃₉H₅₈FN₅O₁₀S, 807.4; m/z found, 808.4 [M+H]⁺.

### Step B—C

Example A70 was synthesized following the same procedures as described in Example A13 (9.4 mg, 66% yield over 2 steps) as a white solid. LC-MS (ESI): mass calcd. for C₆₂H₇₂FN₉O₁₁S, 1169.5; m/z found, 1170.5 [M+H]⁺. ¹H NMR (400 MHz, DMSO-d₆) δ 8.98 (d, J=10.5 Hz, 1H), 8.93 (s, 1H), 8.78 (s, 1H), 8.70 (t, J=5.7 Hz, 1H), 8.48 (t, J=6.0 Hz, 1H), 8.32-8.14 (m, 2H), 8.08 (d, J=8.2 Hz, 1H), 8.04 (s, 1H), 7.86 (d, J=8.5 Hz, 1H), 7.77 (s, 1H), 7.53 (d, J=7.9 Hz, 1H), 7.43-7.37 (m, 2H), 7.35 (s, 1H), 7.28 (d, J=9.1 Hz, 1H), 7.04-7.01 (m, 1H), 6.95 (d, J=7.8 Hz, 1H), 4.68 (s, 2H), 4.59 (d, J=9.4 Hz, 1H), 4.53-4.50 (m, 1H), 4.36-4.34 (m, 1H), 4.27-4.21 (m, 2H), 4.18-4.15 (m, 3H), 3.79-3.76 (m, 2H), 3.66-3.54 (m, 12H), 3.50-3.47 (m, 2H), 2.73-2.60 (m, 3H), 2.44 (s, 3H), 2.11-2.06 (m, 1H), 1.95-1.88 (m, 2H), 1.39-1.33 (m, 2H), 1.23-1.19 (m, 2H), 1.12-1.05 (m, 2H), 0.95 (s, 9H), 0.84-0.77 (m, 2H).

### Example A71. 2-amino-3-ethynyl-N—((S)-6-(6-(((1S,4S)-4-(2-(5-ethynyl-2-(((2S,4R)-1-((S)-2-(1-fluorocyclopropane-1-carboxamido)-3,3-dimethylbutano-yl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

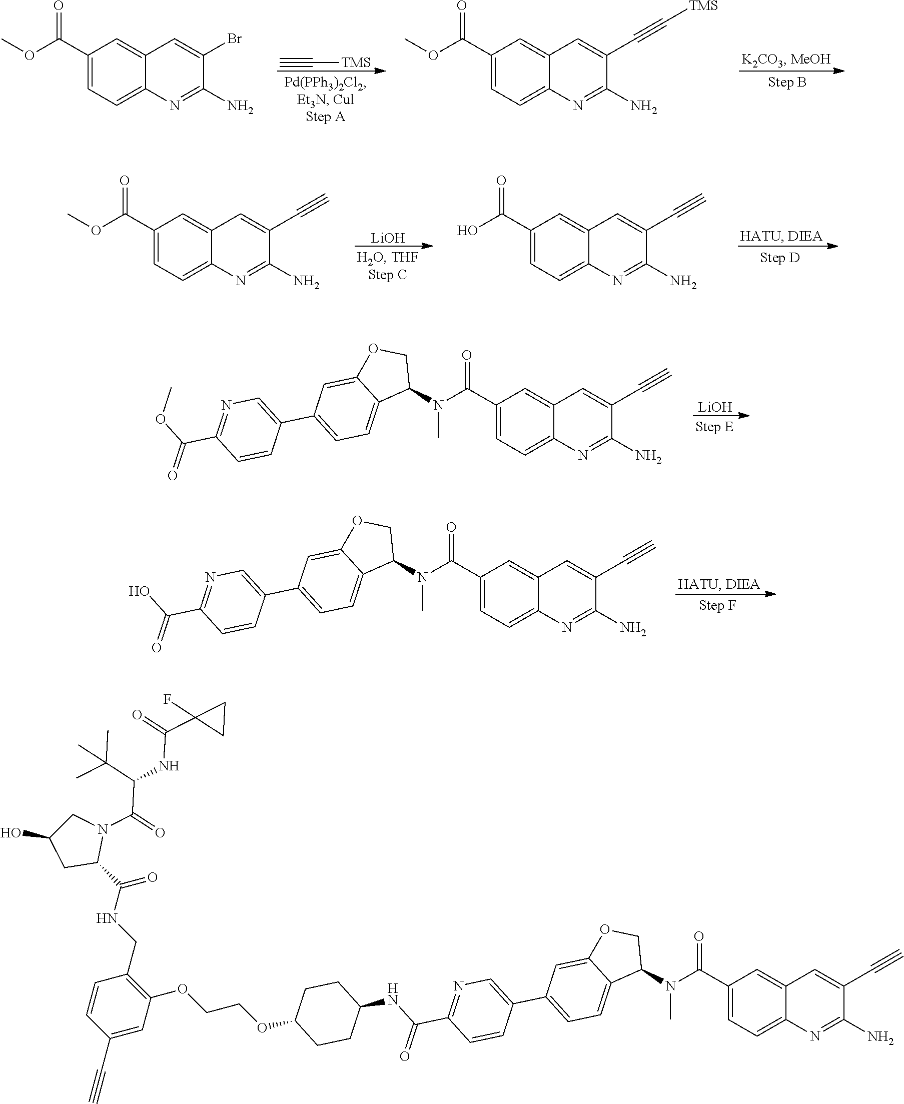

Step A: methyl 2-amino-3-((trimethylsilyl) ethynyl)quinoline-6-carboxylate

To a solution of methyl 2-amino-3-bromoquinoline-6-carboxylate (150 mg, 534 μmol, 1.0 eq.), Bis-(triphenylphosphino)-palladous chloride (56 mg, 80.0 μmol, 0.15 eq.) and Pcy₃ (30 mg, 107 μmol, 0.2 eq.) in DMF (3.0 mL) was added TEA (744 μL, 5.34 mmol, 10.0 eq.) and ethynyltrimethylsilane (314 mg, 3.20 mmol, 6.0 eq.) at 90° C. The mixture was stirred at 90° C. for 6 hours. The resulting solution was diluted with 30 mL of water and extracted with EtOAc (20 mL×3). The combined organic layers were washed with brine (50 mL), dried over Na₂SO₄ and concentrated under reduced pressure. The residue was purified by flash column chromatography on silica gel (100-200 mesh silica gel, 0-5% EtOAc in PE) to give methyl 2-amino-3-((trimethylsilyl) ethynyl)quinoline-6-carboxylate (65 mg, 37% yield) as a yellow solid. LC-MS (ESI): mass calcd, for C16H18N2O₂Si, 298.11; m/z found, 299.0 [M+H]⁺.

Step B: methyl 2-amino-3-ethynylquinoline-6-carboxylate

To a solution of methyl 2-amino-3-((trimethylsilyl) ethynyl)quinoline-6-carboxylate (57 mg, 191 μmol, 1.0 eq.) in MeOH (3.0 mL) was added K₂CO₃ (53 mg, 382 μmol, 2.0 eq.). The mixture was stirred at room temperature for 2 hours. The resulting solution was diluted with 10 mL of water and extracted with EtOAc (10 mL×3). The combined organic layers were washed with brine (20 mL), dried over Na₂SO₄ and concentrated under reduced pressure. The residue was purified by flash column chromatography on silica gel (100-200 mesh silica gel, 0-3% MeOH in DCM) to give methyl 2-amino-3-ethynylquinoline-6-carboxylate (45 mg, 94% yield) as a white solid. LC-MS (ESI): mass calcd, for C₁₃H₁₀N₂O₂, 226.07; m/z found, 227.1 [M+H]⁺.

Step C: 2-amino-3-ethynylquinoline-6-carboxylic acid

To a solution of methyl 2-amino-3-ethynylquinoline-6-carboxylate (45 mg, 199 μmol, 1.0 eq.) in THF (2.0 mL) and Water (2.0 mL) was added Lithium hydroxide monohydrate (17 mg, 398 μmol, 2.0 eq.) and stirred at room temperature for 6 hours. The resulting solution was concentrated under reduced pressure to give 2-amino-3-ethynylquinoline-6-carboxylic acid (40 mg, 95% yield) as a yellow solid. LC-MS (ESI): mass calcd, for C₁₂H₈N₂O₂, 212.06; m/z found, 213.0 [M+H]⁺.

### Step D-F

Example A71 was synthesized following the same procedures as described in Example A41 (6.4 mg, 5% yield over 3 steps) as a white solid. LC-MS (ESI): mass calcd, for C₅₉H₆₃FN₈O₉, 1046.47; m/z found, 1047.7 [M+H]⁺. ¹H NMR (400 MHz, DMSO-d₆) δ 8.93 (s, 1H), 8.57-8.42 (m, 3H), 8.28 (d, J=8.1 Hz, 1H), 8.09 (d, J=8.1 Hz, 1H), 7.99 (s, 1H), 7.82-7.78 (m, 1H), 7.66 (s, 1H), 7.56 (d, J=7.8 Hz, 1H), 7.40 (d, J=7.2 Hz, 1H), 7.36-7.25 (m, 3H), 7.06 (s, 1H), 6.98 (d, J=7.5 Hz, 1H), 6.37-5.71 (m, 1H), 4.82 (s, 1H), 4.70 (s, 1H), 4.59 (d, J=9.1 Hz, 1H), 4.50 (t, J=8.0 Hz, 1H), 4.35 (s, 1H), 4.30-4.19 (m, 2H), 4.17-4.12 (m, 3H), 3.84-3.78 (m, 3H), 3.68-3.60 (m, 4H), 2.93 (s, 1H), 2.67 (s, 3H), 2.10-2.00 (m, 3H), 1.96-1.89 (m, 1H), 1.89-1.81 (m, 2H), 1.56-1.47 (m, 2H), 1.40-1.31 (m, 3H), 1.26-1.22 (m, 3H), 0.95 (s, 9H).

### Example A72. 2-amino-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrroli-dine-2-carboxamido)methyl)phenoxy)ethoxy)cyclohexyl)carbamoyl)pyridin-3-yl)-2,3-dihydrobenzofuran-3-yl)-3-isopropyl-N-methylquinoline-6-carboxamide

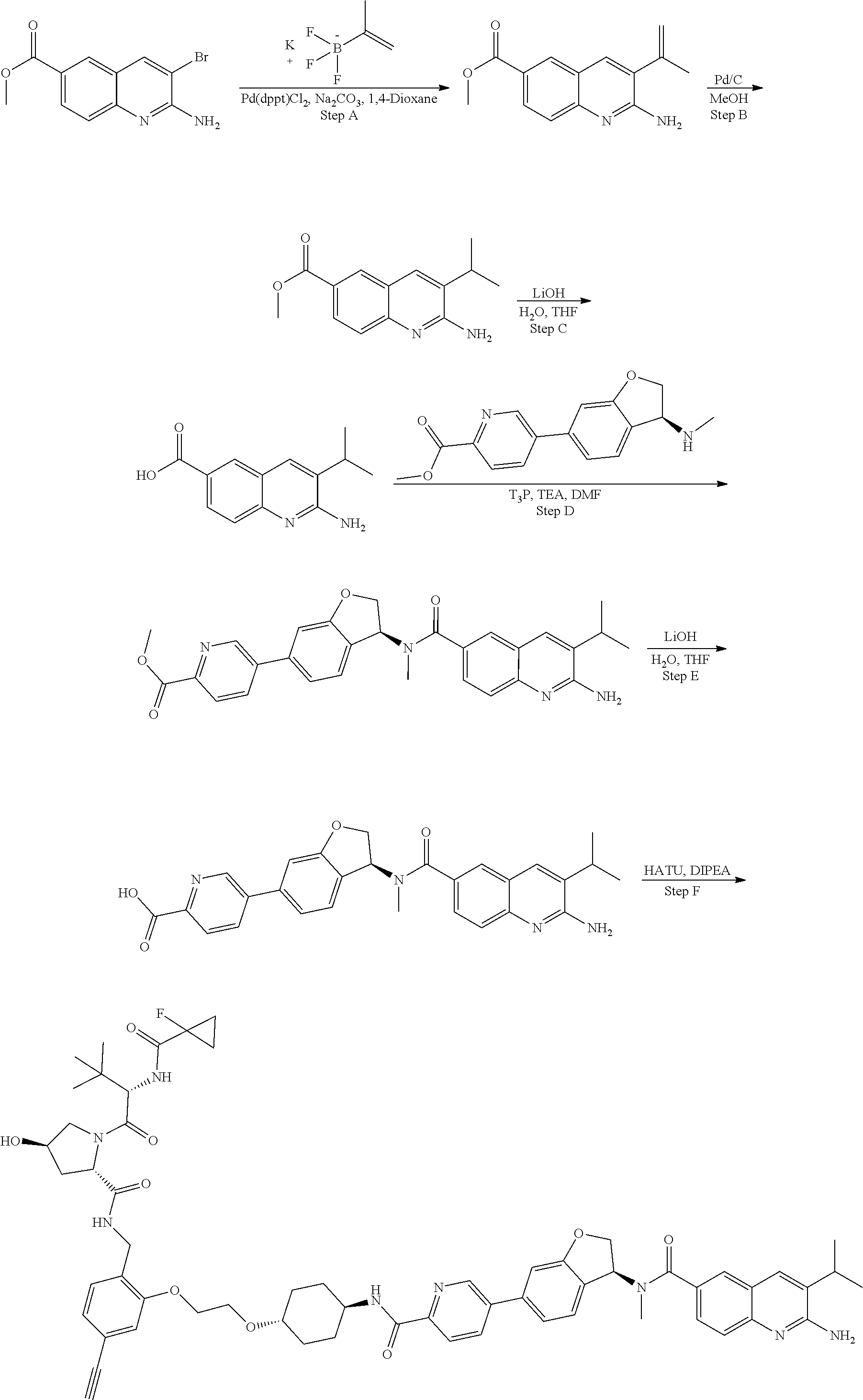

Step A: methyl 2-amino-3-(prop-1-en-2-yl)quinoline-6-carboxylate

To a mixture of methyl 2-amino-3-bromoquinoline-6-carboxylate (100 mg, 356 μmol, 1.0 eq), Pd(dppf)Cl₂ (52 mg, 71 μmol, 0.2 eq), potassium trifluoro (prop-1-en-2-yl) borate (263 mg, 2 mmol, 5.0 eq) and Water (500 μL) in 1,4-Dioxane (5.00 mL) was added Na₂CO₃ (113 mg, 1 mmol, 3.0 eq). The mixture was stirred at 100° C. for 16 hours under N₂ atmosphere. The resulting solution was diluted with 100 mL of water and extracted with EtOAc (30 mL×2). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl 2-amino-3-(prop-1-en-2-yl)quinoline-6-carboxylate (60 mg, 70% yield) as a yellow solid.

LC-MS (ESI): mass calcd, for C₁₄H₁₄N₂O₂, 242.1; m/z found, 243.1 [M+H]⁺.

Step B: methyl 2-amino-3-isopropylquinoline-6-carboxylate

A mixture of methyl 2-amino-3-(prop-1-en-2-yl)quinoline-6-carboxylate (60 mg, 248 μmol, 1.0 eq) and Pd/C(20 mg, 188 μmol, 0.8 eq) in MeOH (5.00 mL) was stirred at room temperature for 16 hour under N₂ atmosphere. The resulting solution was filtered and concentrated under reduced pressure to afford methyl 2-amino-3-isopropylquinoline-6-carboxylate (60 mg, 99% yield) as a yellow solid, which was used directly without further purification.

LC-MS (ESI): mass calcd, for C₁₄H₁₆N₂O₂, 244.1; m/z found, 245.1 [M+H]⁺.

Step C: 2-amino-3-isopropylquinoline-6-carboxylic acid

To a solution of methyl 2-amino-3-isopropylquinoline-6-carboxylate (60 mg, 246 μmol, 1.0 eq) and Water (1.00 mL) in THF (2.00 mL) was added lithium hydroxide (12 mg, 491 μmol, 2.0 eq). The mixture was stirred at room temperature for 16 hours under N₂ atmosphere. The resulting solution was concentrated under reduced pressure to afford 2-amino-3-isopropylquinoline-6-carboxylic acid (55 mg, 97% yield) as a white solid, which was used directly without further purification.

LC-MS (ESI): mass calcd, for C₁₃H₁₄N₂O₂, 230.1; m/z found, 231.1 [M+H]⁺.

### Steps D-F

Example A72 was synthesized following the same procedures as described in Example A41 (2.6 mg, 10% yield over 3 steps) as a white solid. LC-MS (ESI): mass calcd. for C₆₀H₆₉FN₈O₉, 1064.5; m/z found, 1065.5 [M+H]⁺. ¹H NMR (400 MHz, DMSO-d₆) δ 8.92 (s, 1H), 8.53-8.45 (m, 2H), 8.29-8.25 (m, 1H), 8.08 (d, J=8.2 Hz, 1H), 7.93-7.82 (m, 2H), 7.59-7.53 (m, 2H), 7.52-7.46 (m, 1H), 7.39 (d, J=7.9 Hz, 1H), 7.34-7.25 (m, 3H), 7.07-7.05 (m, 1H), 6.98 (d, J=7.7 Hz, 1H), 6.63 (s, 2H), 5.17 (d, J=3.6 Hz, 1H), 4.87-4.69 (m, 1H), 4.69-4.63 (m, 1H), 4.59 (d, J=9.1 Hz, 1H), 4.50 (t, J=8.2 Hz, 1H), 4.35 (s, 1H), 4.30-4.16 (m, 2H), 4.15-4.10 (m, 3H), 3.86-3.80 (m, 1H), 3.80-3.76 (m, 2H), 3.73-3.44 (m, 2H), 3.09-3.03 (m, 1H), 2.68 (s, 3H), 2.07-1.95 (m, 4H), 1.89-1.83 (m, 2H), 1.54-1,47 (m, 2H), 1.39-1.30 (m, 4H), 1.26 (s, 4H), 1.24 (s, 6H), 0.95 (s, 9H).

### Example A73. 2-amino-3-cyclopropyl-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-N-methylquinoline-6-carboxamide

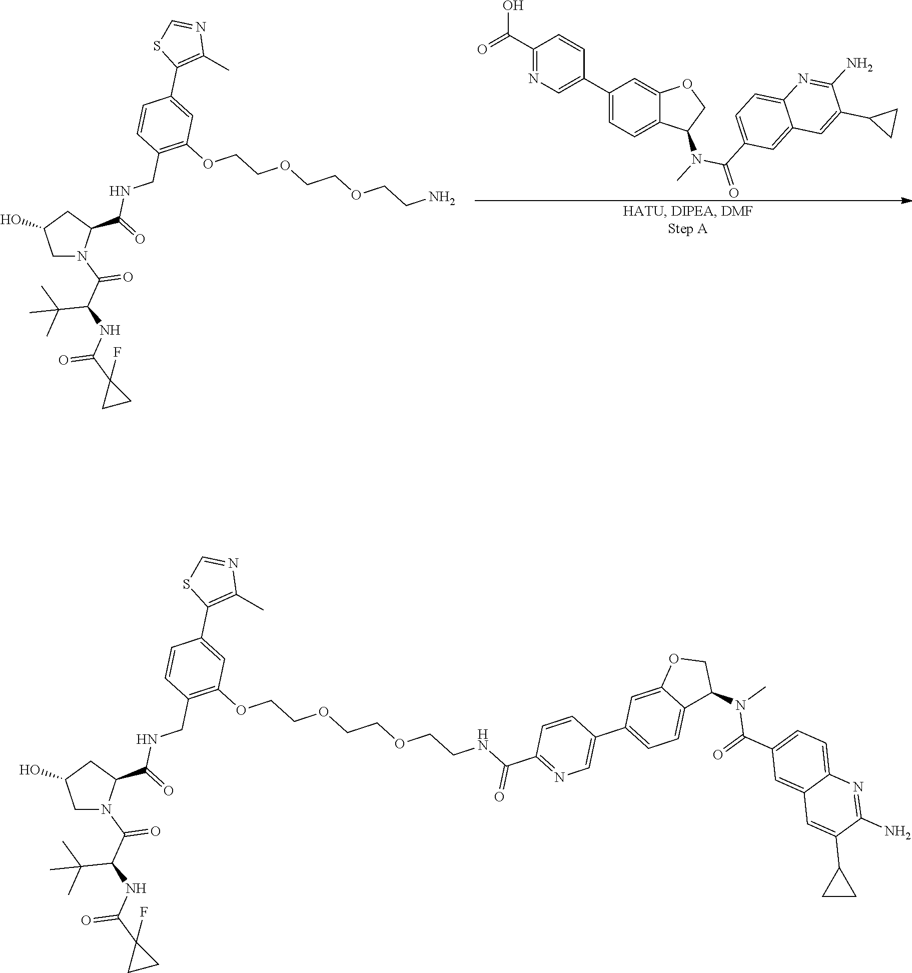 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCN)c1 ; CN(C(=O)c1ccc2nc(N)c(C3CC3)cc2c1)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c(C5CC5)cc4c3)cn2)c1]

Example A73 was synthesized following the same procedures as described in Example A9 (6.2 mg, 18% yield) as a white solid. LC-MS (ESI): mass calcd, for C₆₀H₆₈FN₉O₁₀S, 1125.5; m/z found, 1126.5 [M+H]⁺. ¹H NMR (400 MHz, DMSO-d₆) δ 8.95 (s, 1H), 8.91 (s, 1H), 8.76 (s, 1H), 8.71-8.68 (m, 1H), 8.48 (t, J=6.0 Hz, 1H), 8.27-8.20 (m, 2H), 8.10-8.03 (m, 2H), 7.86 (d, J=8.4 Hz, 1H), 7.79-7.72 (m, 1H), 7.52 (d, J=7.6 Hz, 1H), 7.41-7.32 (m, 3H), 7.28 (d, J=6.6 Hz, 1H), 7.01 (s, 1H), 6.95 (d, J=7.9 Hz, 1H), 4.83-4.64 (m, 2H), 4.59 (d, J=9.2 Hz, 1H), 4.51 (t, J=8.0 Hz, 1H), 4.35-4.33 (m, 1H), 4.28 (d, J=6.0 Hz, 1H), 4.22 (d, J=5.1 Hz, 1H), 4.18-4.15 (m, 2H), 3.81-3.80 (m, 2H), 3.65-3.63 (m, 4H), 3.61-3.60 (m, 3H), 3.51-3.50 (m, 4H), 2.69-2.61 (m, 3H), 2.43 (s, 3H), 2.11-2.05 (m, 1H), 1.94-1.88 (m, 2H), 1.38-1.32 (m, 2H), 1.23-1.19 (m, 2H), 1.10-1.06 (m, 2H), 0.94 (s, 9H), 0.83-0.79 (m, 2H).

### Example A74. N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N,3-dimethylquinoline-6-carboxamide

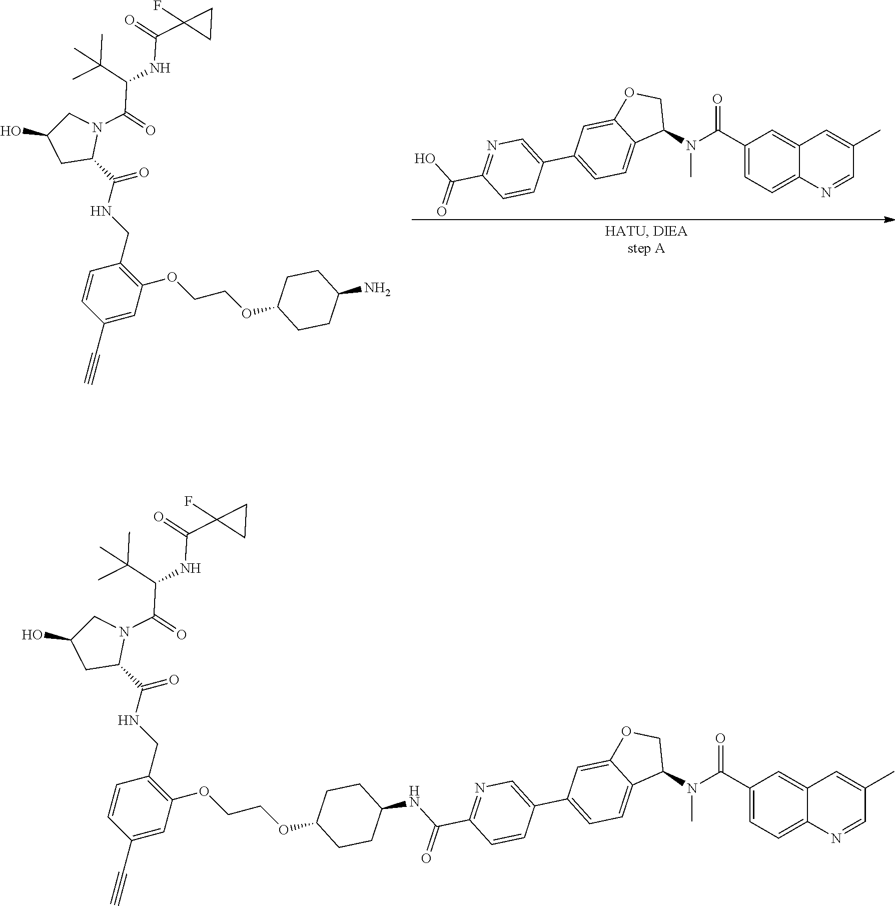 [SMILES: C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](N)CC2)c1 ; Cc1cnc2ccc(C(=O)N(C)[C@@H]3COc4cc(-c5ccc(C(=O)O)nc5)ccc43)cc2c1 ; C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5ncc(C)cc5c4)cn3)CC2)c1]

Example A74 was synthesized following the same procedures as described in Example A39 (9 mg, 18% yield) as a white solid. LC-MS (ESI): mass calcd, for C₅₈H₆₄FN₇O₉, 1021.5; m/z found, 1022.2 [M+H]⁺, ¹H NMR (400 MHz, DMSO-d₆) δ 8.98-8.85 (m, 2H), 8.56-8.44 (m, 2H), 8.35 (s, 1H), 8.28 (d, J=7.2 Hz, 1H), 8.16-8.03 (m, 3H), 7.85 (d, J=8.3 Hz, 1H), 7.59 (d, J=7.7 Hz, 1H), 7.47-7.24 (m, 4H), 7.09-7.06 (m, 1H), 6.99-6.96 (m, 1H), 4.72 (dd, J=10.2, 4.0 Hz, 1H), 4.59 (d, J=9.3 Hz, 1H), 4.50 (t, J=8.2 Hz, 1H), 4.35 (s, 1H), 4.30 (d, J=6.0 Hz, 1H), 4.26 (d, J=6.1 Hz, 1H), 4.21 (d, J=5.6 Hz, 1H), 4.17-4.16 (m, 1H), 4.14-4.12 (m, 4H), 3.80-3.77 (m, 2H), 3.67-3.63 (m, 1H), 3.62-3.58 (m, 1H), 3.39-3.31 (m, 1H), 2.74-2.69 (m, 1H), 2.68-2.61 (m, 2H), 2.54 (s, 3H), 2.12-1.99 (m, 3H), 1.95-1.80 (m, 3H), 1.57-1.46 (m, 2H), 1.42-1.27 (m, 4H), 1.24-1.18 (m, 2H), 0.95 (s, 9H).

### Example A75. 2-amino-3-cyclopropyl-N—((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-methylquinoline-6-carboxamide

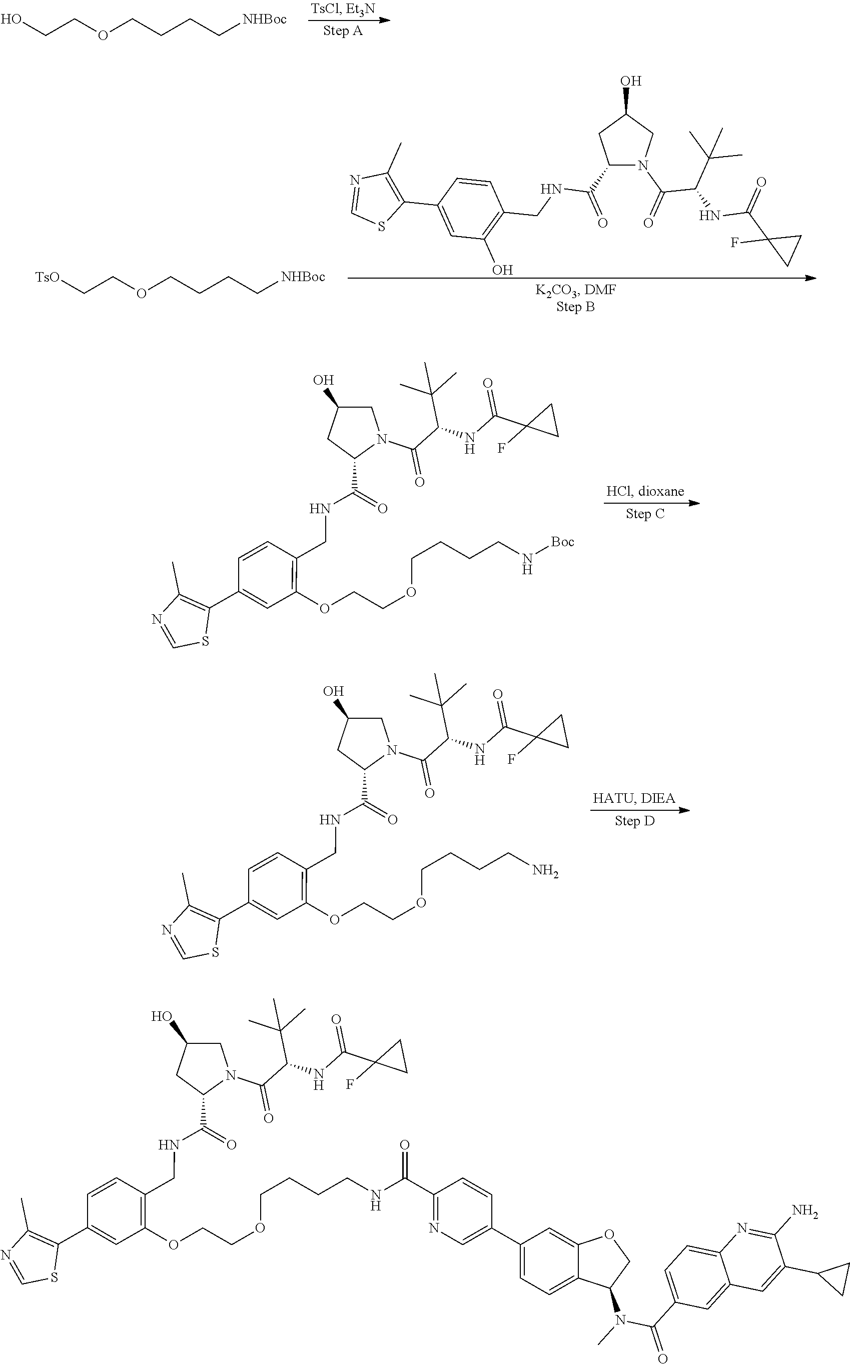

Example A75 was synthesized following the same procedures as described in Example A23 (9.8 mg, 17% yield over 4 steps) as a white solid. LC-MS (ESI): mass calcd. for C₆₀H₆₈FN₉O₉S, 1109.5; m/z found, 1110.5 [M+H]⁺. ¹H NMR (400 MHz, DMSO-d₆) δ 8.97 (s, 1H), 8.91 (s, 1H), 8.81-8.78 (m, 1H), 8.74-8.63 (m, 1H), 8.50-8.44 (m, 1H), 8.28-8.20 (m, 2H), 8.09-8.03 (m, 2H), 7.88-7.84 (m, 1H), 7.80-7.71 (m, 1H), 7.56-7.51 (m, 1H), 7.41-7.34 (m, 3H), 7.30-7.25 (m, 1H), 7.04 (s, 1H), 6.97-6.93 (m, 1H), 4.82-4.66 (m, 2H), 4.60-4.57 (m, 1H), 4.53-4.49 (m, 1H), 4.35-4.33 (m, 1H), 4.30-4.27 (m, 1H), 4.23-4.21 (m, 1H), 4.21-4.17 (m, 3H), 3.67-3.57 (m, 6H), 3.54-3.51 (m, 2H), 3.35-3.30 (m, 2H), 2.67 (s, 3H), 2.45 (s, 3H), 2.10-2.05 (m, 1H), 1.94-1.87 (m, 2H), 1.62-1.55 (m, 4H), 1.38-1.32 (m, 2H), 1.23-1.18 (m, 2H), 1.10-1.06 (m, 2H), 0.94 (s, 9H), 0.83-0.79 (m, 2H).

### Example A76. 3-cyclopropyl-N-(cyclopropylmethyl)-N—((S)-6-(6-(((1S,4S)-4-(2-(2-(((28,4R)-1-((S)-2-(1-fluorocyclopropane-1-carboxamido)-3,3-dimethylbutano-yl)-4-hydroxypyrrolidine-2-carboxamido)methyl)-5-(4-methylthiazol-5-yl)pheno-xy)ethoxy) cyclohexyl)carbamoyl)pyridin-3-yl)-2,3-dihydrobenzofuran-3-yl)quinoline-6-carboxamide

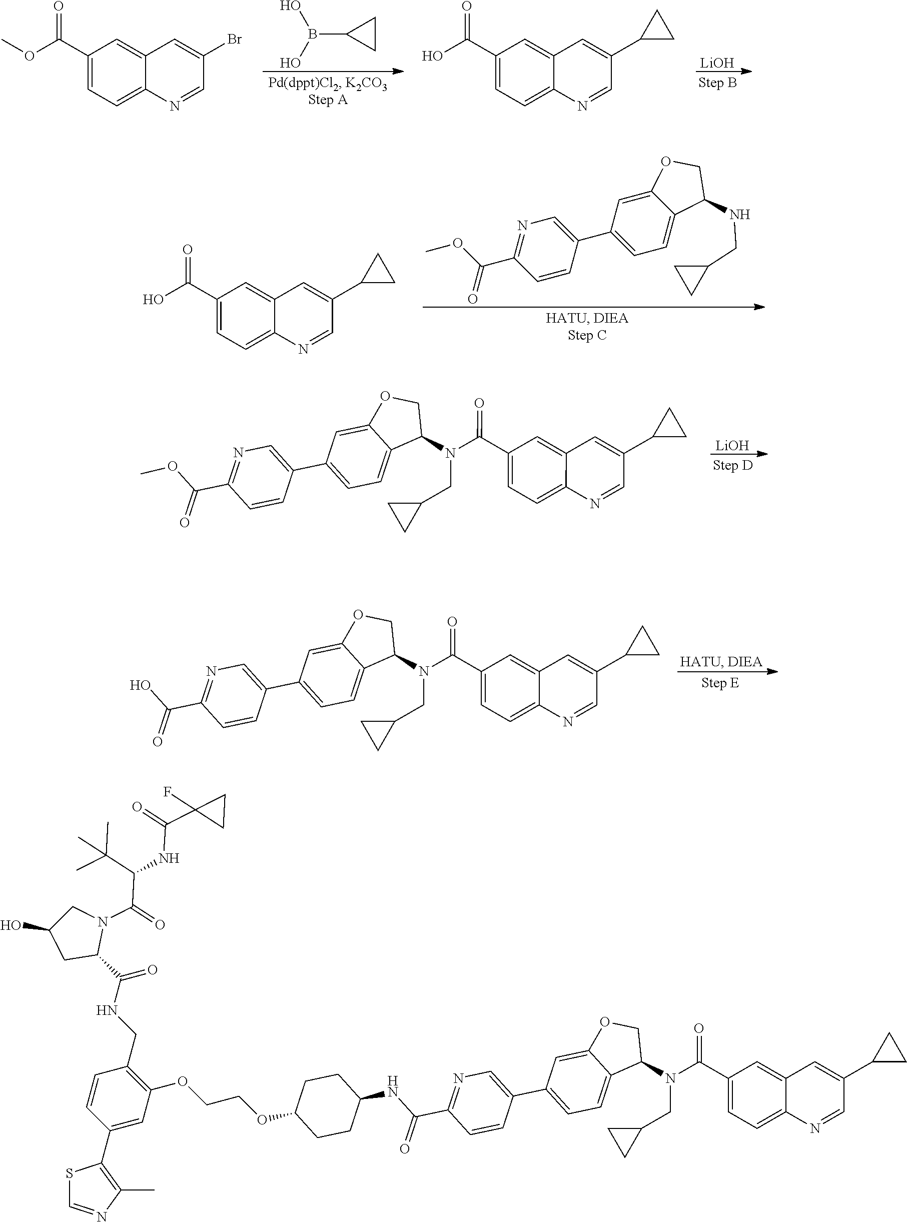

Step A: methyl 2-amino-3-cyclopropylquinoline-6-carboxylate

To a solution of methyl 2-amino-3-bromoquinoline-6-carboxylate (1.0 g, 3.56 mmol, 1.0 eq.), cyclopropylboronic acid (917 mg, 10.7 mmol, 3.0 eq.) and K₂CO₃ (1.5 g, 10.7 mmol, 3.0 eq.) in 1,4-Dioxane (8.0 mL) and Water (2.0 mL) was added PdCl₂ (dppf)(260 mg, 356 μmol, 0.1 eq.). The mixture was stirred at 100° C. under N₂ for 16 hours. The resulting solution was diluted with 100 mL of water and extracted with EtOAc (30 mL×3). The combined organic layers were washed with brine (100 mL), dried over Na₂SO₄ and concentrated under reduced pressure. The residue was purified by flash column chromatography on silica gel (100-200 mesh silica gel, 0-45% EtO Ac in PE) to give methyl 2-amino-3-cyclopropylquinoline-6-carboxylate (370 mg, 43% yield) as a yellow solid.

LC-MS (ESI): mass calcd, for C₁₄H₁₄N₂O₂, 242.10; m/z found, 243.0 [M+H]⁺.

Step B: 2-amino-3-cyclopropylquinoline-6-carboxylic acid

To a solution of methyl 2-amino-3-cyclopropylquinoline-6-carboxylate (370 mg, 1.53 mmol, 1.0 eq.) in THF (5.0 mL) and Water (5.0 mL) was added Lithium hydroxide monohydrate (128 mg, 3.05 mmol, 2.0 eq.) and stirred at room temperature for 16 hours. The resulting solution was concentrated under reduced pressure to 2-amino-3-cyclopropylquinoline-6-carboxylic acid (350 mg, 99% yield) as a yellow solid.

LC-MS (ESI): mass calcd, for C₁₃H₁₁NO₂, 213.1; m/z found, 214.2 [M+H]⁺.

Step C: methyl(S)-5-(3-(3-cyclopropyl-N-(cyclopropylmethyl)quinoline-6-carboxamido)-2,3-dihydrobenzofuran-6-yl)picolinate

To a solution of methyl(S)-5-(3-((cyclopropylmethyl)amino)-2,3-dihydrobenzofuran-6-yl)picolinate (50 mg, 154 μmol, 1.0 eq.) in DMF (3 mL) was added 3-cyclopropylquinoline-6-carboxylic acid (33 mg, 154 μmol, 1.0 eq.), HATU (117 mg, 308 μmol, 2.0 eq.) and DIPEA (80 mg, 617 μmol, 4.0 eq.) at room temperature. The mixture was then stirred at room temperature for 1 hour under N₂ atmosphere. The resulting solution was diluted with 30 mL of H₂O and extracted with EtOAc (30 mL×3). The combined organic layers were washed with brine, dried over Na₂SO₄, and concentrated under reduced pressure. The residue was purified by flash column chromatography on silica gel (100-200 mesh silica gel, 0-100% EtOAc in PE) to afford methyl(S) -5-(3-(3-cyclopropyl-N-(cyclopropylmethyl)quinoline-6-carboxamido)-2,3-dihydrobenzofuran-6-yl)picolinate (58 mg, 72% yield) as a yellow oil.

LC-MS (ESI): mass calcd, for C₃₂H₂₉N₃O₄, 519.2; m/z found, 520.4 [M+H]⁺.

Step D: (S)-5-(3-(3-cyclopropyl-N-(cyclopropylmethyl)quinoline-6-carboxamido)-2,3-dihydrobenzofuran-6-yl)picolinic acid

To a solution of methyl(S)-5-(3-(3-cyclopropyl-N-(cyclopropylmethyl)quinoline-6-carboxamido)-2,3-dihydrobenzofuran-6-yl)picolinate (48 mg, 92 μmol, 1.0 eq.) in THF: H₂O (4 mL, 3:1) was added LiOH (4 mg, 185 μmol, 2.0 eq.) at room temperature. The mixture was then stirred at room temperature for 1 hour under N₂ atmosphere. The reaction was concentrated under reduced pressure to give(S)-5-(3-(3-cyclopropyl-N-(cyclopropylmethyl)quinoline-6-carboxamido)-2,3-dihydrobenzo furan-6-yl)picolinic acid (46 mg, crude). The product was used for next step directly without further purification.

LC-MS (ESI): mass calcd, for C₃₁H₂₇N₃O₄, 505.2; m/z found, 506.5 [M+H]⁺.

Step E: 3-cyclopropyl-N-(cyclopropylmethyl)-N—(S)-6-(6-(1S,4S)-4-(2-(2-((2S,4R)-1-(S)-2-(1-fluorocyclopropane-1-carboxamido)-3, 3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)quinoline-6-carboxamide

To a solution of(S)-5-(3-(3-cyclopropyl-N-(cyclopropylmethyl)quinoline-6-carboxamido)-2,3-dihydrobenzofuran-6-yl)picolinic acid (20 mg, 40 μmol, 1 eq.) and (2S,4R)—N-(2-(2-(((1r,4S)-4-aminocyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (27 mg, 40 μmol, 1.0 eq.) in DMF (3 mL) was added HATU (30 mg, 79 μmol, 2.0 eq.) and DIPEA (20 mg, 158 μmol, 4.0 eq.) at room temperature. The mixture was then stirred at room temperature for 1 hour under N₂ atmosphere. The residue was purified by Pre-HPLC(C18, 20~95%, MeCN in H₂O with 0.1% TFA) to afford 3-cyclopropyl-N-(cyclopropylmethyl)-N—((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)quinoline-6-carboxamide (13 mg, 27% yield) as a white solid.

LC-MS (ESI): mass calcd, for C₆₅H₇₃FN₈O₉S, 1160.5; m/z found, 1161.4 [M+H]⁺. 1H NMR (400 MHz, DMSO-d₆) δ 8.99 (s, 1H), 8.94-8.88 (m, 2H), 8.55-8.42 (m, 2H), 8.27 (d, J=8.3 Hz, 1H), 8.19 (s, 1H), 8.08 (d, J=8.4 Hz, 3H), 7.79 (d, J=8.8 Hz, 1H), 7.61 (d, J=7.8 Hz, 1H), 7.44-7.36 (m, 2H), 7.33-7.26 (m, 2H), 7.05 (s, 1H), 6.97 (d, J=9.1 Hz, 1H), 4.76 (s, 1H), 4.60 (d, J=9.3 Hz, 1H), 4.55-4.50 (m, 1H), 4.40-4.27 (m, 3H), 4.26-4.23 (m, 1H), 4.22-4.16 (m, 3H), 3.82-3.80 (m, 3H), 3.68-3.64 (m, 1H), 3.62-3.59 (m, 1H), 3.39-3.34 (m, 1H), 3.21-3.14 (m, 1H), 3.10-3.02 (m, 1H), 2.47 (s, 3H), 2.24-1.99 (m, 5H), 1.97-1.90 (m, 1H), 1.89-1.83 (m, 2H), 1.57-1.46 (m, 2H), 1.40-1.27 (m, 4H), 1.27-1.17 (m, 3H), 1.15-1.11 (m, 2H), 1.00-0.89 (m, 12H), 0.40-0.24 (m, 2H).

### Example A77. 2-amino-N—((S)-6-(6-((2-(2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamoyl)pyridin-3-yl)-2,3-dihydrobenzofuran-3-yl)-3-isopropyl-N-methylquinoline-6-carboxamide

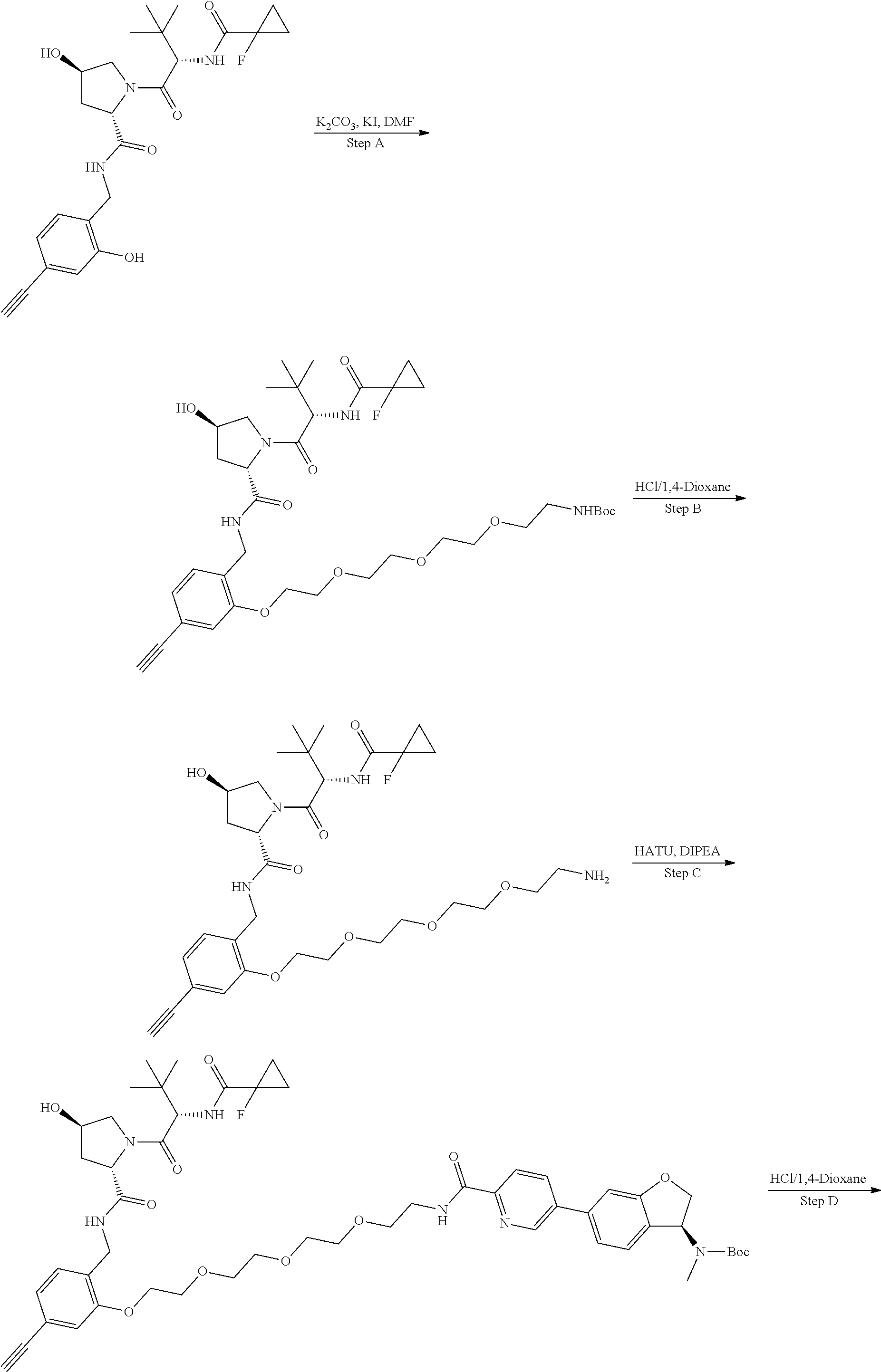

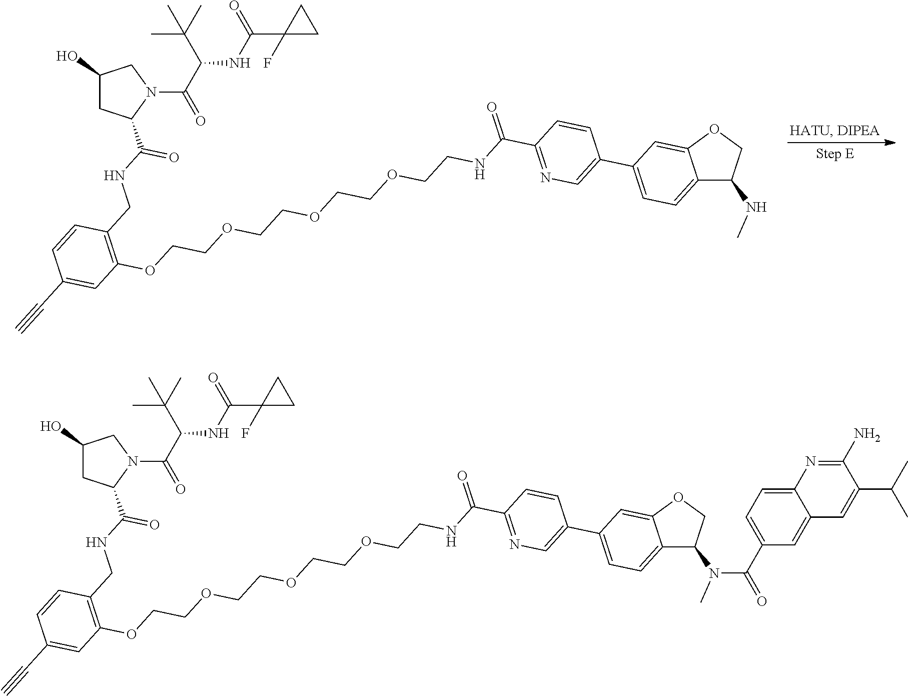 [SMILES: C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4NC)cn2)c1 ; C#Cc1ccc(CNC(=O)[C@@H]2C[C@@H](O)CN2C(=O)[C@@H](NC(=O)C2(F)CC2)C(C)(C)C)c(OCCOCCOCCOCCNC(=O)c2ccc(-c3ccc4c(c3)OC[C@H]4N(C)C(=O)c3ccc4nc(N)c(C(C)C)cc4c3)cn2)c1]

Step A: tert-butyl(2-(2-(2-(2-(5-ethynyl-2-(((25, 4R)-1-((S)-2-(1-fluorocyclopropane-1-carboxamido)-3,3-dimethylbutanoyl)-4-hydroxypyrrolidine-2-carboxamido)methyl) phenoxy)ethoxy)ethoxy)ethoxy)ethyl)carbamate

To a mixture of (2S,4R)—N-(4-ethynyl-2-hydroxybenzyl)-1-((S)-2-(1-fluorocyclopropane-1-carboxamido)-3,3-dimethylbutanoyl)-4-hydroxypyrrolidine-2-carboxamide (30 mg, 65 μmol, 1.0 eq), KI (3 mg, 20 μmol, 0.3 eq) and 2,2-dimethyl-4-oxo-3,8,11,14-tetraoxa-5-azahexadecan-16-yl 4-methylbenzenesulfonate (38 mg, 85 μmol, 1.3 eq) in DMF (3.00 mL) was added K₂CO₃ (27 mg, 196 μmol, 3.0 eq). The mixture was stirred at 75° C. for 3 hours under N₂ atmosphere. The resulting solution was diluted with 100 mL of water and extracted with EtOAc (30 mL×2). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford tert-butyl(2-(2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethoxy)ethyl)carbamate (25 mg, yield 52%) as a colorless oil.

LC-MS (ESI): mass calcd, for C₃₇H₅₅FN₄O₁₀, 734.4; m/z found, 735.4 [M+H]⁺.

### Steps B-E

Example A77 was synthesized following the same procedures as described in Example A39 (4.3 mg, 11% yield over 4 steps) as a white solid. LC-MS (ESI): mass calcd. for C₆₀H₇₁FN₈O₁₁, 1098.5; m/z found, 1099.5 [M+H]⁺. ¹H NMR (400 MHz, DMSO-d₆) δ 8.94 (s, 1H), 8.72-8.67 (m, 2H), 8.51-8.44 (m, 1H), 8.40 (s, 1H), 8.30-8.26 (m, 1H), 8.14-8.07 (m, 2H), 7.89-7.84 (m, 1H), 7.79-7.71 (m, 1H), 7.57-7.53 (m, 1H), 7.43-7.35 (m, 2H), 7.32-7.25 (m, 2H), 7.24-7.07 (m, 1H), 7.03 (s, 1H), 6.97-6.94 (m, 1H), 5.03-4.61 (m, 2H), 4.61-4.56 (m, 1H), 4.53-4.43 (m, 1H), 4.35-4.29 (m, 1H), 4.29-4.20 (m, 1H), 4.20-4.13 (m, 1H), 4.13-4.09 (m, 3H), 3.76-3.73 (m, 2H), 3.64-3.53 (m, 14H), 3.17-3.13 (m, 1H), 2.67 (s, 3H), 2.10-2.04 (m, 1H), 1.92-1.85 (m, 1H), 1.40-1.32 (m, 2H), 1.30-1.27 (m, 6H), 1.23-1.19 (m, 2H), 0.94 (s, 9H).

### Example A78. 2-amino-3-cyclopropyl-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-isopropylquinoline-6-carboxamide

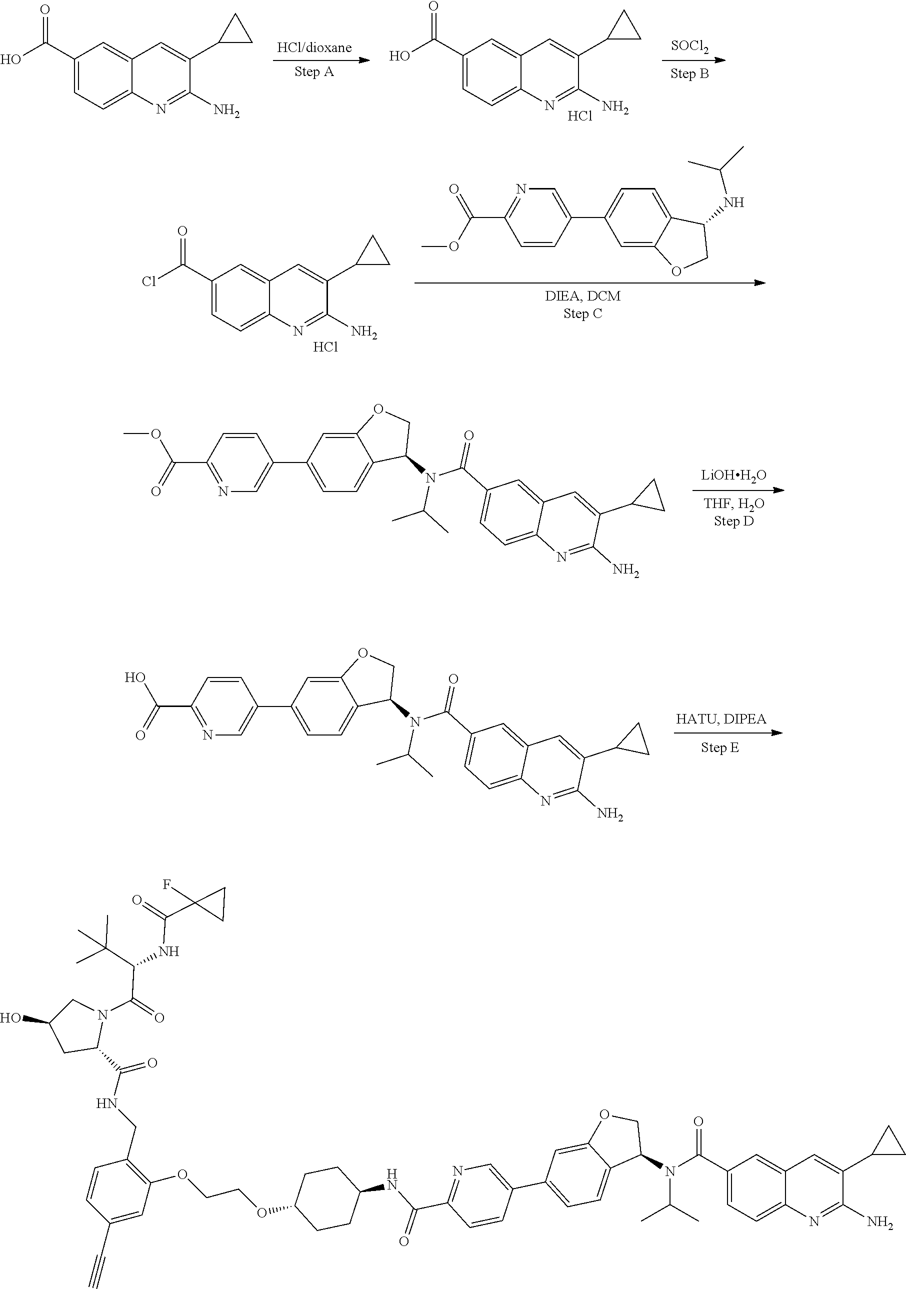

Example A78 was synthesized following the same procedures as described in Example A45 (11 mg, 14% yield over 5 steps) as a white solid. LC-MS (ESI): mass calcd, for C₆₂H₇₁FN₈O₉, 1090.53; m/z found, 1091.5 [M+H]⁺. ¹H NMR (400 MHz, DMSO-d₆DMSO-d₆) δ 8.89 (s, 1H), 8.76-8.54 (m, 2H), 8.53-8.40 (m, 2H), 8.27-8.15 (m, 2H), 8.09-8.04 (m, 1H), 7.97-7.80 (m, 1H), 7.78-7.65 (m, 2H), 7.45-7.34 (m, 1H), 7.33-7.24 (m, 3H), 7.24-7.12 (m, 1H), 7.06 (s, 1H), 6.98 (d, J=8.3 Hz, 1H), 5.48-5.38 (m, 1H), 4.82-4.73 (m, 1H), 4.71-4.65 (m, 1H), 4.59 (d, J=8.9 Hz, 1H), 4.49 (t, J=8.1 Hz, 1H), 4.35 (s, 1H), 4.32-4.25 (m, 1H), 4.23-4.16 (m, 1H), 4.15-4.10 (m, 3H), 3.80-3.77 (m, 2H), 3.65-3.60 (m, 2H), 3.39-3.37 (m, 2H), 2.11-2.00 (m, 3H), 1.95-1.83 (m, 4H), 1.52-1.45 (m, 2H), 1.42-1.37 (m, 3H), 1.36-1.29 (m, 3H), 1.27-1.14 (m, 6H), 1.09-1.04 (m, 2H), 0.99-0.92 (m, 9H), 0.84-0.76 (m, 2H).

### Example A79. N1-(7-amino-1-isopropyl-1H-pyrazolo[3,4-c]pyridin-4-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) ethoxy)ethyl)carbamoyl)pyridin-3-yl)-2,3-dihydrobenzofuran-3-yl)-N2-methyloxalamide

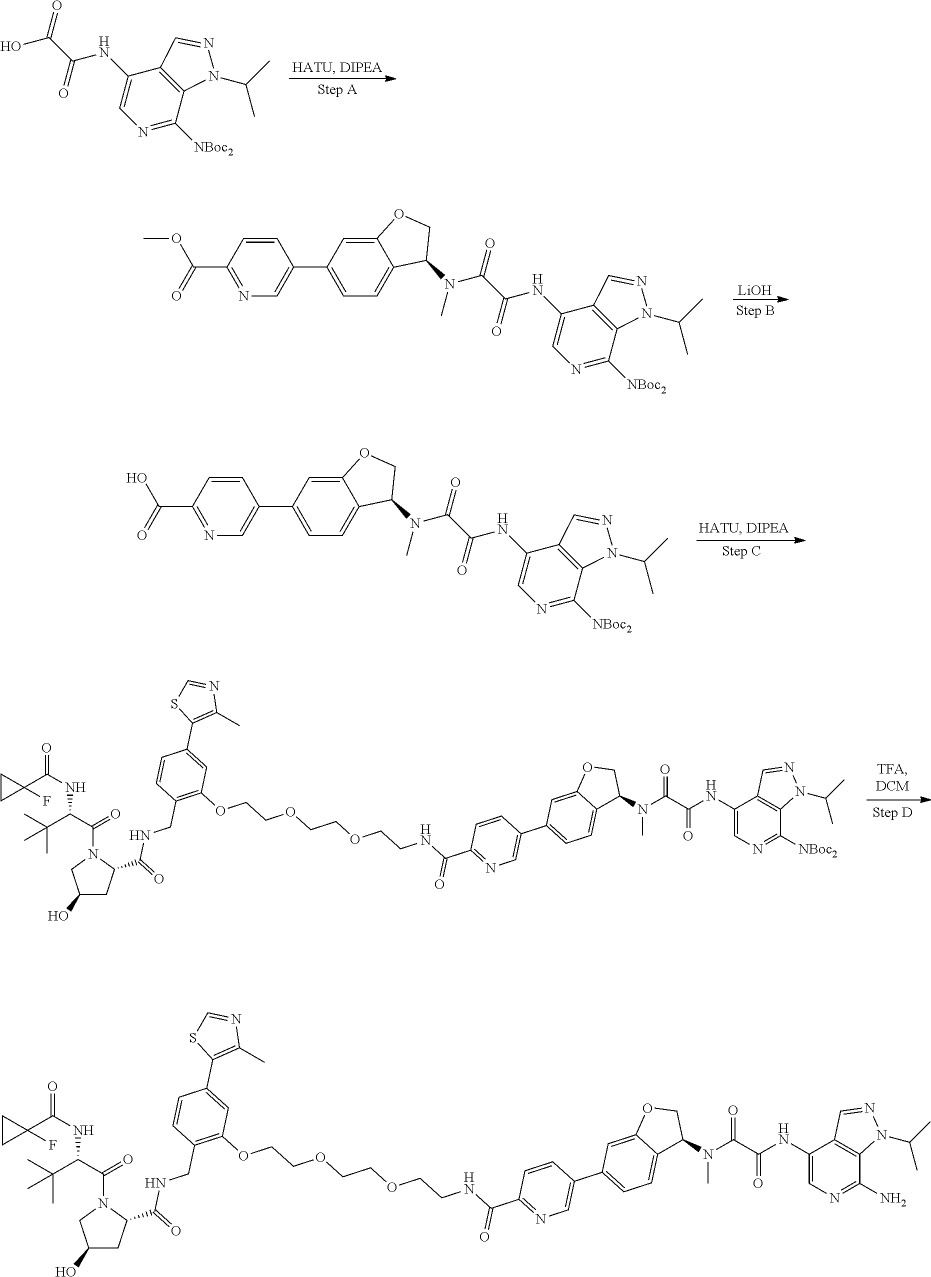

Example A79 was synthesized following the same procedures as described in Example A44 (1.3 mg, 1% yield over 12 steps) as a white solid. LC-MS (ESI): mass calcd. for C₅₈H₆FN₁₂O₁₁S, 1160.49; m/z found, 1161.3 [M+H]⁺. ¹H NMR (400 MHz, DMSO-d₆) δ 10.75 (d, J=61.8 Hz, 1H), 9.01-8.86 (m, 2H), 8.76-8.68 (m, 1H), 8.49-8.42 (m, 2H), 8.26 (d, J=8.1 Hz, 1H), 8.16-8.04 (m, 2H), 8.01-7.87 (m, 1H), 7.66-7.43 (m, 1H), 7.42-7.34 (m, 3H), 7.31-7.26 (m, 1H), 7.01 (s, 1H), 6.95 (d, J=7.7 Hz, 1H), 6.22 (s, 2H), 5.33-5.27 (m, 1H), 5.16 (s, 1H), 4.83-4.72 (m, 1H), 4.70-4.62 (m, 1H), 4.61-4.49 (m, 2H), 4.36-4.32 (m, 1H), 4.29-4.23 (m, 1H), 4.19-4.15 (m, 2H), 3.83-3.78 (m, 2H), 3.67-3.62 (m, 3H), 3.62-3.57 (m, 5H), 3.52-3.47 (m, 3H), 3.37 (s, 3H), 2.43 (s, 3H), 2.12-2.06 (m, 1H), 2.02-1.98 (m, 1H), 1.94-1.89 (m, 1H), 1.46 (d, J=6.4 Hz, 6H), 1,40-1.36 (m, 1H), 1.35-1.32 (m, 1H), 1.30-1.27 (m, 1H), 0.95 (s, 9H).

### Example A80. 2-amino-3-cyclopropyl-N—((S)-6-(1-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)-1H-pyrazol-4-yl)-2,3-dihydrobenzofuran-3-yl)-N-methyl quinoline-6-carboxamide

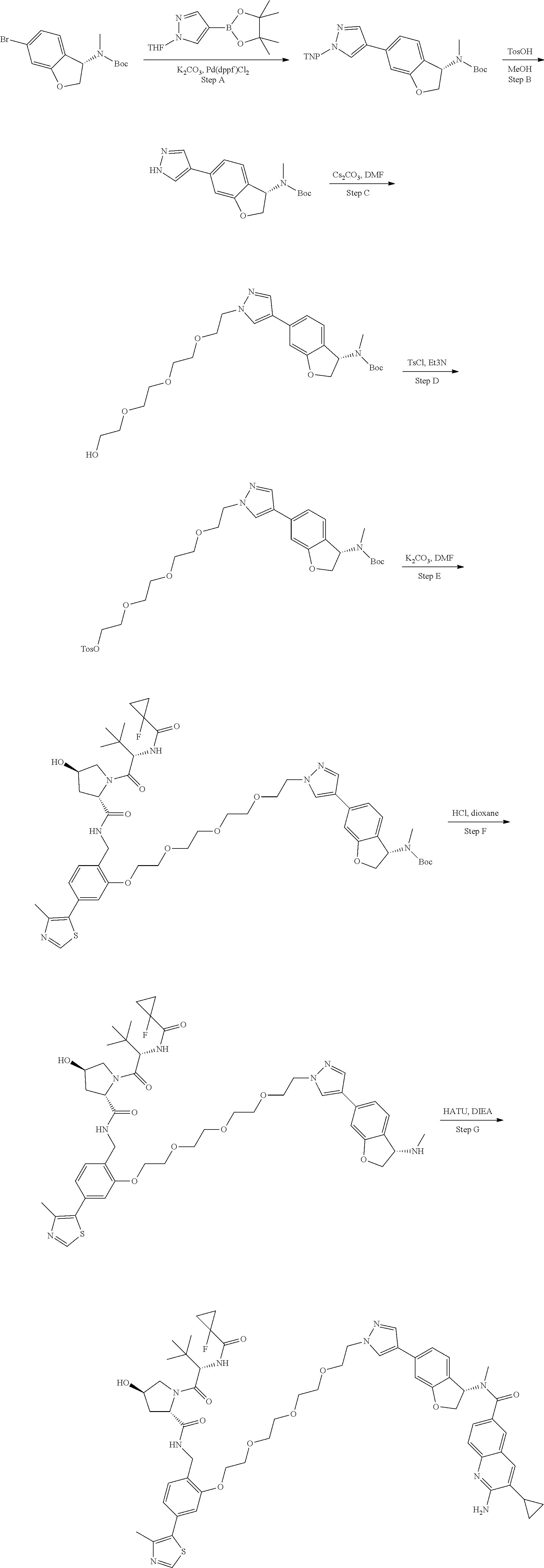

Step A: tert-butyl methyl ((3S)-6-(1-(tetrahydro-2H-pyran-2-yl)-1H-pyrazol-4-yl)-2,3-dihydrobenzofuran-3-yl)carbamate

To a stirred mixture of tert-butyl(S)-(6-bromo-2,3-dihydrobenzofuran-3-yl)(methyl)carbamate (530 mg, 1.61 mmol, 1.0 eq.) in dioxane (5 mL) and H₂O (1.3 mL) were added 1-(tetrahydro-2H-pyran-2-yl)-4-(4,4,5,5-tetramethyl-1,3,2-dioxaborolan-2-yl)-1H-pyrazole (539 mg, 1.94 mmol, 1.2 eq.), potassium carbonate (446 mg, 3.23 mmol, 2.0 eq.) and Pd (dppf) C12 (59 mg, 80.7 μmol, 0.05 eq.). The resulting mixture was stirred at 100° C. under N₂ for 5 hours. The reaction mixture was poured into water and then extracted with EtOAc (50 mL×2). The combined organic layers were washed with brine, dried over anhydrous Na₂SO₄ and concentrated. The residue was purified by column chromatography (silica gel, 0% EtOAc in PE ~ 20% EtOAc in PE) to afford tert-butyl methyl ((3S)-6-(1-(tetrahydro-2H-pyran-2-yl)-1H-pyrazol-4-yl)-2,3-dihydrobenzofuran-3-yl)carbamate (600 mg, 93% yield) as a white solid.

LC-MS (ESI): mass calcd. For C₂₂H₂₉N₃O₄, 399.22; m/z found, 400.3 [M+H]⁺.

Step B: tert-butyl(S)-(6-(1H-pyrazol-4-yl)-2,3-dihydrobenzofuran-3-yl)(methyl)carbamate

To a stirred mixture of tert-butyl methyl ((3S)-6-(1-(tetrahydro-2H-pyran-2-yl)-1H-pyrazol-4-yl)-2,3-dihydrobenzofuran-3-yl)carbamate (600 mg, 1.5 mmol, 1.0 eq.) in MeOH (6 mL) were added p-Toluenesulfonic acid (129 mg, 751.0 μmol, 0.5 eq.). The resulting mixture was stirred at room temperature for 15 hours. The reaction mixture was poured into water and then extracted with EtOAc (50 mL×2). The combined organic layers were washed with brine, dried over anhydrous Na₂SO₄ and concentrated. The residue was triturated with PE: EtOAc (10:1) and filtered to afford tert-butyl(S)-(6-(1H-pyrazol-4-yl)-2,3-dihydrobenzofuran-3-yl)(methyl)carbamate (372 mg, 79% yield) as a white solid.

LC-MS (ESI): mass calcd. For C₁₇H₂₁N₃O₃, 315.16; m/z found, 316.2 [M+H]⁺.

Step C: tert-butyl(S)-(6-(1-(2-(2-(2-(2-hydroxyethoxy)ethoxy)ethoxy)ethyl)-1H-pyrazol-4-yl)-2,3-dihydrobenzofuran-3-yl)(methyl)carbamate

To a stirred mixture of tert-butyl(S)-(6-(1H-pyrazol-4-yl)-2,3-dihydrobenzofuran-3-yl)(methyl)carbamate (372 mg, 1.18 mmol, 1.0 eq.) in DMF (4 mL) were added 2-(2-(2-(2-bromoethoxy)ethoxy)ethoxy) ethan-1-ol (364 mg, 1.42 mmol, 1.2 eq.) and Cs₂CO₃ (769 mg, 2.36 mmol, 2.0 eq.). The resulting mixture was stirred at 60° C. for 2 hours. The reaction mixture was poured into water and then extracted with EtOAc (50 mL×2). The combined organic layers were washed with brine, dried over anhydrous Na₂SO₄ and concentrated. The residue was purified by column chromatography (silica gel, 0% EtOAc in PE~10% methanol in dichloromethane) to afford tert-butyl(S)-(6-(1-(2-(2-(2-(2-hydroxyethoxy)ethoxy)ethoxy)ethyl)-1H-pyrazol-4-yl)-2,3-dihydrobenzofuran-3-yl)(methyl)carbamate (535 mg, 92% yield) as a white solid.

LC-MS (ESI): mass calcd. For C₂₅H₃₇N₃O₇, 491.26; m/z found, 492.4 [M+H]⁺.

Step D: (S)-2-(2-(2-(2-(4-(3-((tert-butoxycarbonyl)(methyl)amino)-2,3-dihydrobenzofuran-6-yl)-1H-pyrazol-1-yl)ethoxy)ethoxy)ethoxy)ethyl 4-methylbenzenesulfonate

To a stirred mixture of tert-butyl(S)-(6-(1-(2-(2-(2-(2-hydroxyethoxy)ethoxy)ethoxy)ethyl)-1H-pyrazol-4-yl)-2,3-dihydrobenzofuran-3-yl)(methyl)carbamate (535 mg, 1.1 mmol, 1.0 eq.) in DCM (5 mL) were added DIEA (422 mg, 3.265 mmol, 3.0 eq.), N,N-dimethylpyridin-4-amine (266 mg, 2.18 mmol, 2.0 eq.) and p-Toluenesulfonyl chloride (311 mg, 1.632 mmol, 1.5 eq.) slowly at 0° C. The resulting mixture was stirred at room temperature for 3 hours. The reaction mixture was poured into water and then extracted with EtOAc (50 mL×2). The combined organic layers were washed with brine, dried over anhydrous Na₂SO₄ and concentrated. The residue was purified by column chromatography (silica gel, 0% EtOAc in PE~ 100% EtOAc in PE) to afford(S)-2-(2-(2-(2-(4-(3-((tert-butoxycarbonyl)(methyl)amino)-2,3-dihydrobenzofuran-6-yl)-1H-pyrazol-1-yl)ethoxy)ethoxy)ethoxy)ethyl 4-methylbenzenesulfonate (446 mg, 64% yield) as a white solid.

LC-MS (ESI): mass calcd. For C32H43N309S, 645.27; m/z found, 646.6 [M+H]⁺.

Step E: tert-butyl ((S)-6-(1-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)-1H-pyrazol-4-yl)-2,3-dihydrobenzofuran-3-yl)(methyl)carbamate

To a stirred mixture of(S)-2-(2-(2-(2-(4-(3-((tert-butoxycarbonyl)(methyl)amino)-2,3-dihydrobenzofuran-6-yl)-1H-pyrazol-1-yl)ethoxy)ethoxy)ethoxy)ethyl 4-methylbenzenesulfonate (446 mg, 690.6 μmol, 1.0 eq.) in DMF (5 mL) were added (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (368 mg, 690.6 μmol, 1.0 eq.) and K₂CO₃ (286 mg, 2.072 mmol, 3.0 eq.). The resulting mixture was stirred at 60° C. for 15 hours. The reaction mixture was poured into water and then extracted with EtOAc (50 mL×2). The combined organic layers were washed with brine, dried over anhydrous Na₂SO₄ and concentrated. The residue was purified by column chromatography (silica gel, 0% EtOAc in PE ~ 10% methanol in dichloromethane) to afford tert-butyl ((S)-6-(1-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)-1H-pyrazol-4-yl)-2,3-dihydrobenzofuran-3-yl)(methyl)carbamate (405 mg, 58% yield) as a white solid.

LC-MS (ESI): mass calcd. For C₅₁H₆₈FN₇O₁₁S, 1005.47; m/z found, 1006.8 [M+H]⁺.

Step F: (2S,4R)-1-((S)-2-(1-fluorocyclopropane-1-carboxamido)-3,3-dimethylbutanoyl)-4-hydroxy-N-(2-(2-(2-(2-(2-(4-((S)-3-(methylamino)-2,3-dihydrobenzofuran-6-yl)-1H-pyrazol-1-yl)ethoxy)ethoxy)ethoxy)ethoxy)-4-(4-methylthiazol-5-yl)benzyl)pyrrolidine-2-carboxamide

To a solution of tert-butyl ((S)-6-(1-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)-1H-pyrazol-4-yl)-2,3-dihydrobenzo furan-3-yl)(methyl)carbamate (405 mg, 403 μmol, 1.0 eq.) in dioxane (4 mL) was added hydrochloric acid (4 mL, 4 M/L), the reaction mixture was stirred at room temperature for 1 hour. The reaction mixture was concentrated under reduced pressure to (2S,4R)-1-((S)-2-(1-fluorocyclopropane-1-carboxamido)-3,3-dimethylbutanoyl)-4-afford hydroxy-N-(2-(2-(2-(2-(2-(4-((S)-3-(methylamino)-2,3-dihydrobenzofuran-6-yl)-1H-pyrazol-1-yl)ethoxy)ethoxy)ethoxy)ethoxy)-4-(4-methylthiazol-5-yl)benzyl)pyrrolidine-2-carboxamide (365 mg, 99% yield) as a white solid.

LC-MS (ESI): mass calcd. For C₄₆H₆₀FN₇O₉S, 905.42; m/z found, 906.7 [M+H]⁺.

Step G: 2-amino-3-cyclopropyl-N—((S)-6-(1-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)-1H-pyrazol-4-yl)-2,3-dihydrobenzofuran-3-yl)-N-methyl quinoline-6-carboxamide

To a stirred mixture of (2S,4R)-1-((S)-2-(1-fluorocyclopropane-1-carboxamido)-3,3-dimethylbutanoyl)-4-hydroxy-N-(2-(2-(2-(2-(2-(4-((S)-3-(methylamino)-2,3-dihydrobenzofuran-6-yl)-1H-pyrazol-1-yl)ethoxy)ethoxy)ethoxy)ethoxy)-4-(4-methylthiazol-5-yl)benzyl)pyrrolidine -2-carboxamide (50 mg, 55.2 μmol, 1.0 eq.) in DMF (2 mL) were added DIEA (35.7 mg, 48.1 μL, 276 μmol, 5.0 eq.), 2-amino-3-cyclopropylquinoline-6-carboxylic acid (13.4 mg, 55.2 μmol, 1.0 eq.) and HATU (21.0 mg, 55.2 μmol, 1.0 eq.). The resulting mixture was stirred at room temperature for 1 hour. The reaction mixture was purified by prep-HPLC to afford 2-amino-3-cyclopropyl-N—((S)-6-(1-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)-1H-pyrazol-4-yl)-2,3-dihydrobenzofuran-3-yl)-N-methyl quinoline-6-carboxamide (25 mg, 38% yield) as a white solid.

LC-MS (ESI): mass calcd, for C₅₉H₇₀FN₉O₁₀S, 1115.50; m/z found, 1116.2 [M+H]⁺. ¹H NMR (400 MHz, DMSO-d₆) δ 8.96 (s, 1H), 8.79-8.71 (m, 2H), 8.48 (t, J=5.9 Hz, 1H), 8.20 (s, 1H), 8.16 (s, 1H), 8.05-7.98 (m, 1H), 7.88 (s, 1H), 7.83 (d, J=7.4 Hz, 1H), 7.78-7.72 (m, 1H), 7.39 (d, J=7.7 Hz, 1H), 7.35-7.31 (m, 1H), 7.28 (d, J=6.9 Hz, 1H), 7.19-7.15 (m, 1H), 7.12-7.07 (m, 1H), 7.01-6.98 (m, 1H), 6.95 (d, J=8.0 Hz, 1H), 4.78-4.57 (m, 3H), 4.53-4.49 (m, 1H), 4.34 (s, 1H), 4.30-4.20 (m, 4H), 4.18-4.11 (m, 3H), 3.78-3.75 (m, 4H), 3.58-3.56 (m, 4H), 3.51-3.48 (m, 6H), 2.66-2.55 (m, 3H), 2.44 (s, 3H), 2.09-2.04 (m, 1H), 1.94-1.88 (m, 2H), 1.39-1.32 (m, 2H), 1.21 (d, J=8.4 Hz, 2H), 1.07 (d, J=8.1 Hz, 2H), 0.95 (s, 9H), 0.83-0.79 (m, 2H).

### Example A81. 4-amino-N—((S)-6-(1-(2-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethoxy)ethyl)-1H-pyrazol-4-yl)-2,3-dihydrobenzofuran-3-yl)-N,1-dimethyl-1H-pyrazolo[4,3-c]quinoline-8-carboxamide

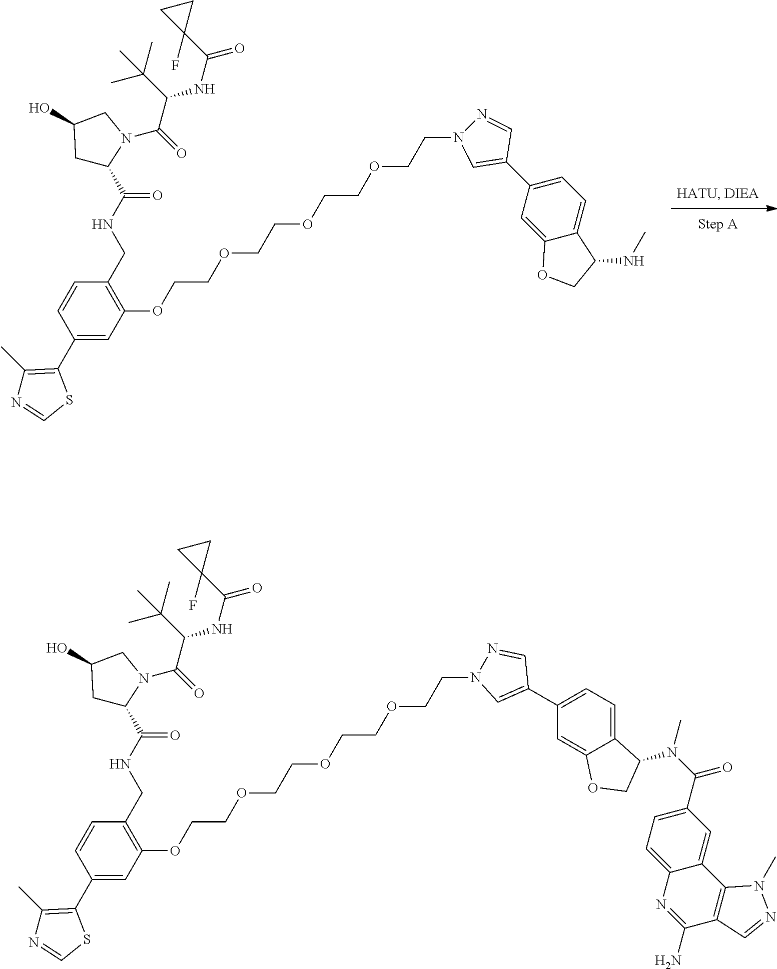

Example A81 was synthesized following the same procedures as described in Example A80 (7.50 mg, 12% yield) as a white solid. LC-MS (ESI): mass calcd. For C₅₈H₆₈FN₁₁O₁₀S, 1129.49; m/z found, 1129.8 [M+H]⁺. ¹H NMR (400 MHz, DMSO-d₆) δ 9.63 (s, 1H), 8.96 (s, 1H), 8.63 (s, 1H), 8.48 (t, J=5.8 Hz, 1H), 8.42 (s, 1H), 8.16 (s, 1H), 7.92-7.81 (m, 3H), 7.43-7.32 (m, 2H), 7.28 (d, J=6.7 Hz, 1H), 7.18 (d, J=5.6 Hz, 1H), 7.10 (s, 1H), 7.00 (s, 1H), 6.93 (d, J=7.8 Hz, 1H), 4.70-4.64 (m, 1H), 4.59 (d, J=9.1 Hz, 1H), 4.53 (d, J=3.7 Hz, 1H), 4.51-4.47 (m, 3H), 4.34 (s, 1H), 4.32-4.26 (m, 1H), 4.26-4.19 (m, 3H), 4.18-4.09 (m, 3H), 3.79-3.77 (m, 2H), 3.75-3.73 (m, 2H), 3.63-3.62 (m, 1H), 3.61-3.60 (m, 1H), 3.59-3.56 (m, 4H), 3.52-3.51 (m, 2H), 3.49 (s, 3H), 2.51 (s, 3H), 2.43 (s, 3H), 2.10-2.03 (m, 1H), 1.94-1.87 (m, 1H), 1.40-1.30 (m, 2H), 1.21 (dd, J=8.3, 2.6 Hz, 2H), 0.95 (s, 9H).

### Example A82. 2-amino-3-cyclopropyl-N—((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-isopropylquinoline-6-carboxamide

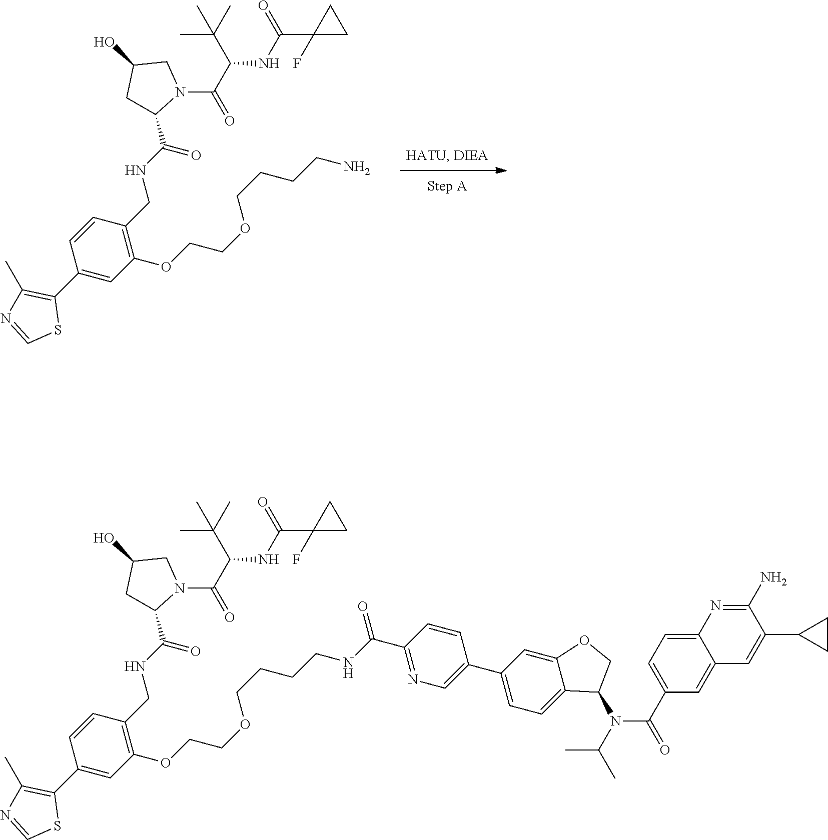 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C(=O)c3ccc4nc(N)c(C5CC5)cc4c3)C(C)C)cn2)c1]

Example A82 was synthesized following the same procedures as described in Example A28 (15.3 mg, 44% yield) as a white solid. LC-MS (ESI): mass calcd, for C₆₂H₇₂FN₉O₉S, 1137.52; m/z found, 1138.3 [M+H]⁺. ¹H NMR (400 MHz, DMSO-d₆DMSO-d₆) δ 8.97 (s, 1H), 8.88 (s, 1H), 8.78 (d, J=6.0 Hz, 1H), 8.74-8.68 (m, 1H), 8.48 (t, J=5.9 Hz, 1H), 8.26-8.15 (m, 2H), 8.06 (d, J=8.2 Hz, 1H), 7.88 (s, 1H), 7.78-7.65 (m, 2H), 7.40 (d, J=7.7 Hz, 1H), 7.38-7.31 (m, 1H), 7.31-7.25 (m, 2H), 7.23-7.14 (m, 1H), 7.04 (s, 1H), 6.95 (d, J=7.8 Hz, 1H), 5.42 (t, J=9.1 Hz, 1H), 4.82-4.73 (m, 1H), 4.70-4.65 (m, 1H), 4.58 (d, J=9.1 Hz, 1H), 4.50 (t, J=8.1 Hz, 1H), 4.38-4.30 (m, 2H), 4.28 (d, J=5.8 Hz, 1H), 4.24-4.21 (m, 1H), 4.20-4.15 (m, 3H), 3.75-3.74 (m, 2H), 3.53-3.49 (m, 4H), 3.35-3.30 (m, 2H), 2.45 (s, 3H), 2.10-2.04 (m, 1H), 1.94-1.86 (m, 2H), 1.63-1.56 (m, 4H), 1.42-1.37 (m, 3H), 1.35-1.29 (m, 2H), 1.25-1.15 (m, 5H), 1.09-1.04 (m, 2H), 0.94 (s, 9H), 0.82-0.75 (m, 2H).

### Example A83. 2-amino-3-chloro-N—((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

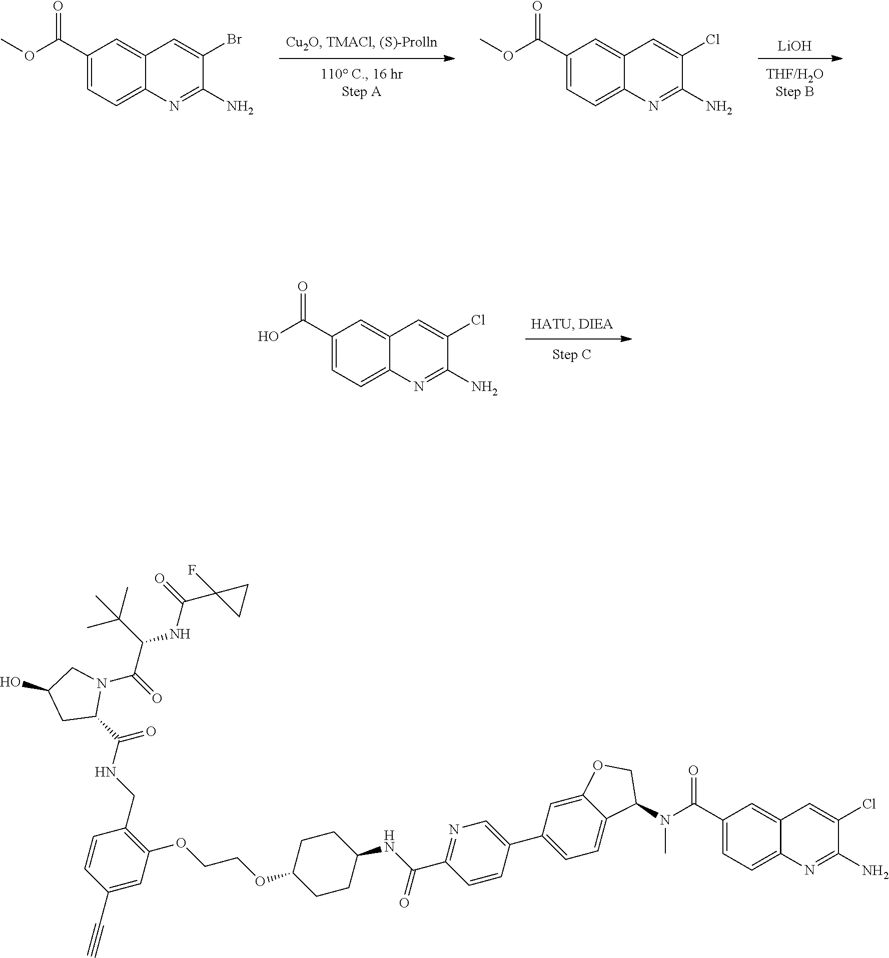 [SMILES: COC(=O)c1ccc2nc(N)c(Br)cc2c1 ; "Cu2O, TMACl, (S)-Prolln" ; COC(=O)c1ccc2nc(N)c(Cl)cc2c1 ; "LiOH" ; Nc1nc2ccc(C(=O)O)cc2cc1Cl ; C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(Cl)cc5c4)cn3)CC2)c1]

Step A: methyl 2-amino-3-chloroquinoline-6-carboxylate

A mixture of methyl 2-amino-3-bromoquinoline-6-carboxylate (150 mg, 534 μmol, 1.0 eq.), (S)-(−)-Proline (184 mg 1.60 mmol, 3.0 eq.), Tetramethylammonium chloride (468 mg, 4.27 mmol, 8.0 eq.) and Cu₂O (81 mg, 800 μmol, 1.5 eq.) in 1,4-Dioxane (10 mL) was stirred at 110° C. for 16 hours under nitrogen. The resulting solution was diluted with 20 mL of water and extracted with EtOAc (30 mL×3). The combined organic layers were washed with brine (30 mL), dried over Na₂SO₄ and concentrated under reduced pressure. The crude product was purified by flash column chromatography on silica gel (100-200 mesh silica gel, 0 ~ 10% MeOH in DCM) to afford methyl 2-amino-3-chloroquinoline-6-carboxylate (10 mg, 8% yield) as a yellow solid.

LC-MS (ESI): mass calcd. For C₁₁H₉ClN₂O₂, 236.04; m/z found, 237.0 [M+H]⁺.

Step B: 2-amino-3-chloroquinoline-6-carboxylic acid

A mixture of methyl 2-amino-3-chloroquinoline-6-carboxylate (20 mg, 84.5 μmol, 1.0 eq.) and LiOH (4.0 mg, 169 μmol, 2.0 eq.) in THF (1.00 mL) and H₂O (1.0 mL) was stirred at room temperature for 1 hour under nitrogen. The resulting solution was removed under reduced pressure to afford 2-amino-3-chloroquinoline-6-carboxylic acid (18 mg, 96% yield) as a yellow solid.

LC-MS (ESI): mass calcd. For C₁₀H₇ClN₂O₂, 222.02; m/z found, 223.0 [M+H]⁺.

Step C: 2-amino-3-chloro-N—(S)-6-(6-(((15,4S)-4-(2-(5-ethynyl-2-(((28,4R)-1-(S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

To a stirred mixture of N-((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)-5-((S)-3-(methylamino)-2,3-dihydrobenzofuran-6-yl)picolinamide (25 mg, 29.2 μmol, 1.0 eq.) in DMF (2 mL) were added DIEA (19 mg, 27.1 μL, 146 μmol, 5.0 eq.), 2-amino-3-chloroquinoline-6-carboxylic acid (6.5 mg, 29.2 μmol, 1.0 eq.) and HATU (22.3 mg, 58.4 μmol, 2.0 eq.). The resulting mixture was stirred at room temperature for 1 hour. The reaction mixture was purified by prep-HPLC to afford 2-amino-3-chloro-N—((S)-6-(6-(((1S,4S)-4-(2-(5-ethynyl-2-(((2S,4R)-1-((S)-2-(1-fluorocyclopropane-1-carboxamido)-3,3-dimethyl-butanoyl)-4-hydroxypyrr-olidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide (3 mg, 8% yield) as a white solid.

LC-MS (ESI): mass calcd, for C₅₇H₆₂ClFN₈O₉, 1056.43; m/z found, 1057 [M+H]⁺. ¹H NMR (400 MHz, DMSO-d₆) δ 8.92 (s, 1H), 8.51-8.46 (m, 2H), 8.33-8.25 (m, 2H), 8.08 (d, J=8.2 Hz, 1H), 7.88 (s, 1H), 7.68-7.63 (m, 1H), 7.55 (d, J=7.6 Hz, 2H), 7.39 (d, J=7.8 Hz, 1H), 7.35-7.27 (m, 3H), 7.06 (s, 1H), 6.98 (d, J=6.5 Hz, 3H), 5.17 (d, J=3.6 Hz, 1H), 4.83-4.65 (m, 2H), 4.59 (d, J=9.3 Hz, 1H), 4.53-4.47 (m, 1H), 4.35 (s, 1H), 4.30-4.17 (m, 2H), 4.15-4.11 (m, 3H), 3.83-3.77 (m, 3H), 3.67-3.60 (m, 2H), 3.31-3.27 (m, 2H), 2.67 (s, 3H), 2.07-2.00 (m, 3H), 1.94-1.83 (m, 3H), 1.55-1.47 (m, 2H), 1.39-1.29 (m, 4H), 1.28-1.20 (m, 4H), 0.97-0.94 (m, 9H).

### Example A84. 2-amino-3-ethynyl-N—((S)-6-(6-(((1S,4S)-4-(2-(2-(((25,4S)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

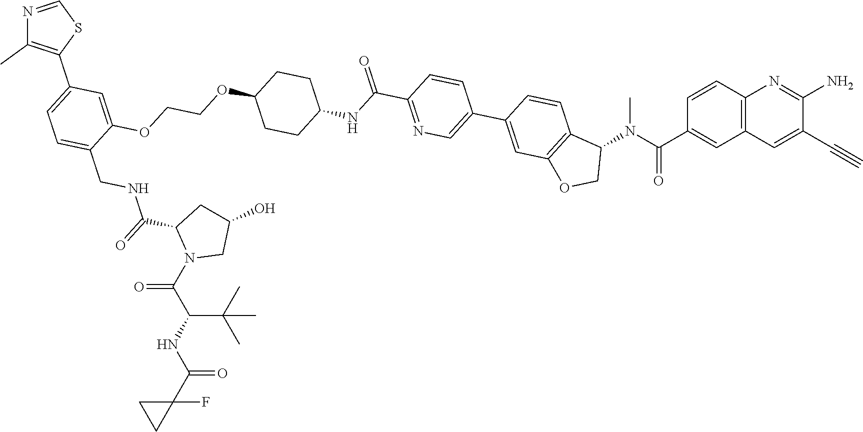 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@H](O)CN7C(=O)[C@@H](NC(=O)C7(F)CC7)C(C)(C)C)CC6)nc5)ccc43)ccc2nc1N]

Example A84 was synthesized following the same procedures as described in Example A1 using (2S,4S)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide as the coupling partner. It is a diastereomeric isomer of Example A357. LC-MS (ESI): mass calcd. for C₆₁H₆₆ClFN₉O₉S,1119.47; m/z found, 1120 [M+H]⁺. ¹H NMR (400 MHz, DMSO) δ 8.99 (s, 1H), 8.92 (s, 1H), 8.65 (s, 1H), 8.57 (t, J=6.0 Hz, 1H), 8.49 (d, J=8.4 Hz, 1H), 8.43 (s, 1H), 8.27 (d, J=9.8 Hz, 1H), 8.14-7.99 (m, 2H), 7.88 (d, J=8.2 Hz, 1H), 7.74 (s, 1H), 7.56 (d, J=7.7 Hz, 1H), 7.47-7.23 (m, 4H), 7.06 (s, 1H), 6.96 (d, J=7.8 Hz, 1H), 4.93 (s, 1H), 4.71-4.68 (m, 1H), 4.56-4.53 (m, 1H), 4.46-4.43 (m, 1H), 4.39-4.35 (m, 1H), 4.34-4.31 (m, 1H), 4.27-4.23 (m, 2H), 4.19-4.17 (m, 2H), 3.89-3.84 (m, 2H), 3.83-3.79 (m, 3H), 3.49-3.43 (m, 1H), 3.40-3.34 (m, 1H), 2.67 (s, 3H), 2.47 (s, 3H), 2.42-2.32 (m, 1H), 2.10-2.02 (m, 2H), 1,92-1.82 (m, 2H), 1.81-1.73 (m, 1H), 1.58-1.44 (m, 2H), 1.40-1.26 (m, 4H), 1.24-1.17 (m, 2H), 0.97 (s, 9H).

### Example A85. 2-amino-3-cyclopropyl-N—((S)-6-(6-((4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)carbamoyl)pyridin-3-yl)-2,3-dihydrobenzofuran-3-yl)-N-methylquinoline-6-carboxamide

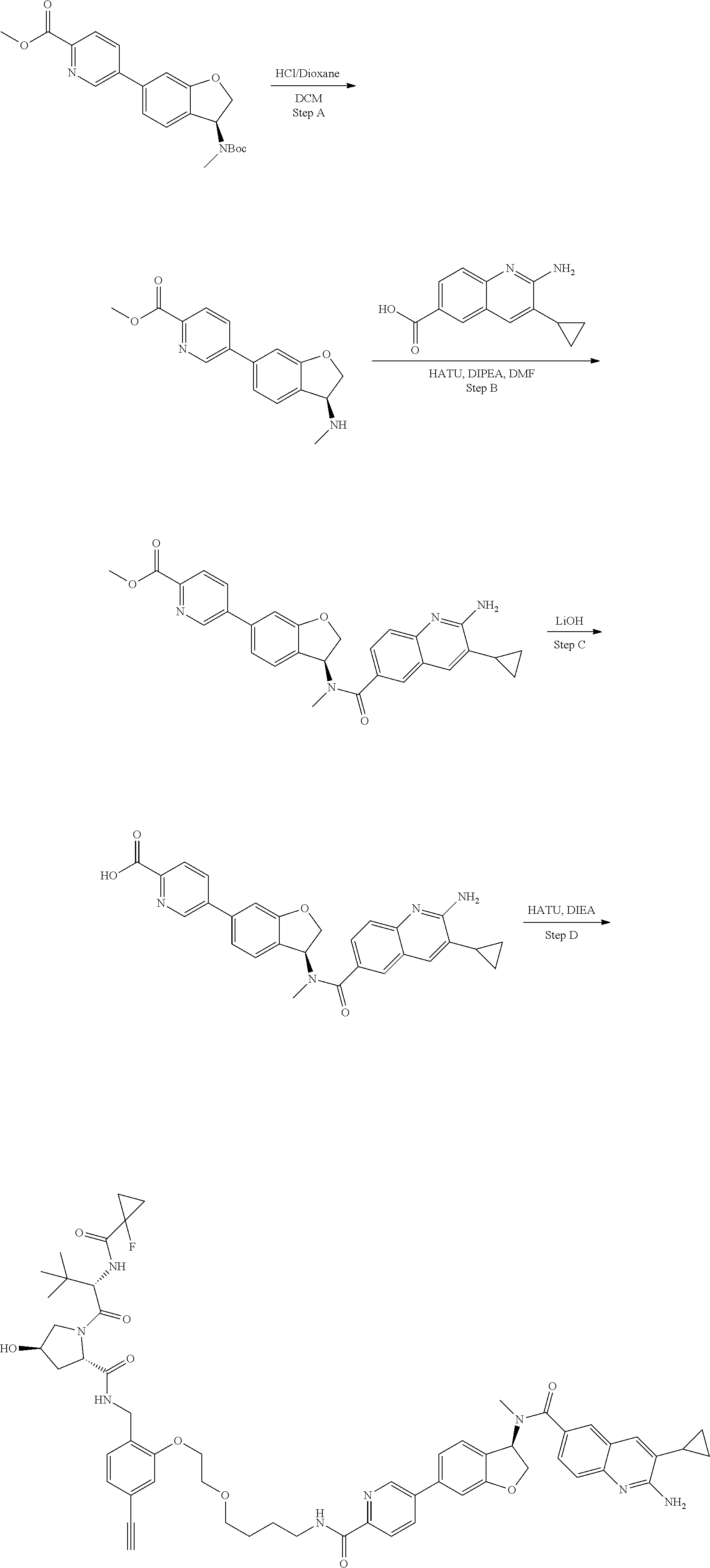

Step A: methyl(S)-5-(3-(methylamino)-2,3-dihydrobenzofuran-6-yl)picolinate

To a solution of methyl(S)-5-(3-((tert-butoxycarbonyl)(methyl)amino)-2,3-dihydrobenzofuran-6-yl)picolinate (200 mg, 520 μmol, 1.0 eq) in DCM (3.00 mL) was added HCl/Dioxane (1.00 mL). The mixture was stirred at 0° C. for 30 min under N₂ atmosphere. The resulting solution was concentrated under reduced pressure to afford methyl(S)-5-(3-(methylamino)-2,3-dihydrobenzofuran-6-yl)picolinate (148 mg, 99% yield) as a white solid, which was used directly without further purification.

LC-MS (ESI): mass calcd, for C₁₆H₁₆N₂O₃, 284.1; m/z found, 285.1 [M+H]⁺.

Step B: methyl(S)-5-(3-(2-amino-3-cyclopropyl-N-methylquinoline-6-carboxamido)-2,3-dihydrobenzofuran-6-yl)picolinate

To a mixture of methyl(S)-5-(3-(methylamino)-2,3-dihydrobenzofuran-6-yl)picolinate (148 mg, 521 μmol, 1.0 eq), HATU (396 mg, 1 mmol, 2.0 eq) and 2-amino-3-cyclopropylquinoline-6-carboxylic acid (143 mg, 625 μmol, 1.2 eq) in DMF (2.00 mL) was added DIEA (269 mg, 2 mmol, 4.0 eq). The mixture was stirred at room temperature for 30 mins under N2 atmosphere. The resulting solution was diluted with 100 mL of water and extracted with EtOAc (30 mL×3). The combined organic layers were washed with brine, dried over anhydrous Na₂SO₄ and concentrated under reduced pressure. The residue was purified by flash column chromatography on silica gel (100-200 mesh silica gel, 0-100% EtOAc in PE) to afford methyl(S) -5-(3-(2-amino-3-cyclopropyl-N-methylquinoline-6-carboxamido)-2,3-dihydrobenzofuran-6-yl)picolinate (180 mg, 70% yield) as a yellow solid.

LC-MS (ESI): mass calcd, for C₂₉H₂₆N₄O₄, 494.2; m/z found, 495.2 [M+H]⁺.

Step C: (S)-5-(3-(2-amino-3-cyclopropyl-N-methylquinoline-6-carboxamido)-2,3-dihydrobenzofuran-6-yl)picolinic acid

To a solution of methyl(S)-5-(3-(2-amino-3-cyclopropyl-N-methylquinoline-6-carboxamido)-2,3-dihydrobenzofuran-6-yl)picolinate (180 mg, 364 μmol, 1.0 eq) in THF (2.0 mL) and Water (1.0 mL) was added lithium hydroxide (17.4 mg, 728 μmol, 2.0 eq). The mixture was stirred at room temperature for 1 hour under N₂ atmosphere. The resulting solution was concentrated under reduced pressure to afford(S)-5-(3-(2-amino-3-cyclopropyl-N-methylquinoline-6-carboxamido)-2,3-dihydrobenzofuran-6-yl)picolinic acid (175 mg, 99% yield) as a white solid, which was used directly without further purification.

LC-MS (ESI): mass calcd, for C₂₈H₂₄N₄O₄, 480.2; m/z found, 481.2 [M+H]⁺.

Step D: 2-amino-3-cyclopropyl-N—((R)-6-(6-((4-(2-(5-ethynyl-2-(((25, 4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)carbamoyl)pyridin-3-yl)-2,3-dihydrobenzofuran-3-yl)-N-methylquinoline-6-carboxamide

To a mixture of (2S,4R)—N-(2-(2-(4-aminobutoxy)ethoxy)-4-ethynylbenzyl)-1-((S)-2-(1-fluorocyclopropane-1-carboxamido)-3,3-dimethylbutanoyl)-4-hydroxypyrrolidine-2-carboxamide (36 mg, 62 μmol, 1.5 eq), HATU (32 mg, 83 μmol, 2.0 eq) and(S)-5-(3-(2-amino-3-cyclopropyl-N-methylquinoline-6-carboxamido)-2,3-dihydrobenzo furan-6-yl)picolinic acid (20 mg, 42 μmol, 1.0 eq) in DMF (2 mL) was added DIEA (22 mg, 166 μmol, 4.0 eq). The mixture was stirred at 0° C. for 30 minutes under N₂ atmosphere. The resulting solution was purified by Prep-HPLC to afford 2-amino-3-cyclopropyl-N—((S)-6-(6-((4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)carbamoyl)pyridin-3-yl)-2,3-dihydrobenzofuran-3-yl)-N-methylquinoline-6-carboxamide (5.4 mg, 13% yield) as a white solid.

LC-MS (ESI): mass calcd, for C₅₈H₆₅FN₈O₉, 1036.5; m/z found, 1037.5 [M+H]⁺. ¹H NMR (400 MHz, DMSO-d₆) δ 8.91 (s, 1H), 8.83-8.72 (m, 3H), 8.51-8.42 (m, 1H), 8.28-8.24 (m, 1H), 8.21 (s, 1H), 8.10-8.07 (m, 1H), 8.04 (s, 1H), 7.88-7.84 (m, 1H), 7.81-7.73 (m, 1H), 7.56-7.52 (m, 1H), 7.44-7.38 (m, 1H), 7.36 (s, 1H), 7.32-7.29 (m, 1H), 7.29-7.24 (m, 1H), 7.05 (s, 1H), 6.98-6.95 (m, 1H), 4.87-4.64 (m, 2H), 4.60-4.55 (m, 1H), 4.51-4.45 (m, 1H), 4.36-4.31 (m, 1H), 4.30-4.16 (m, 2H), 4.15-4.11 (m, 3H), 3.76-3.71 (m, 2H), 3.64-3.58 (m, 2H), 3.52-3.50 (m, 2H), 3.36-3.33 (m, 2H), 2.65 (s, 3H), 2.10-2.03 (m, 1H), 1.93-1.86 (m, 2H), 1.62-1.53 (m, 4H), 1.41-1.32 (m, 2H), 1.23-1.19 (m, 2H), 1.10-1.06 (m, 2H), 0.93 (s, 9H), 0.83-0.78 (m, 2H).

### Example A86. 2-amino-N—((S)-6-(6-((4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)carbamoyl)pyridin-3-yl)-2,3-dihydrobenzofuran-3-yl)-N-isopropyl-3-methylquinoline-6-carboxamide

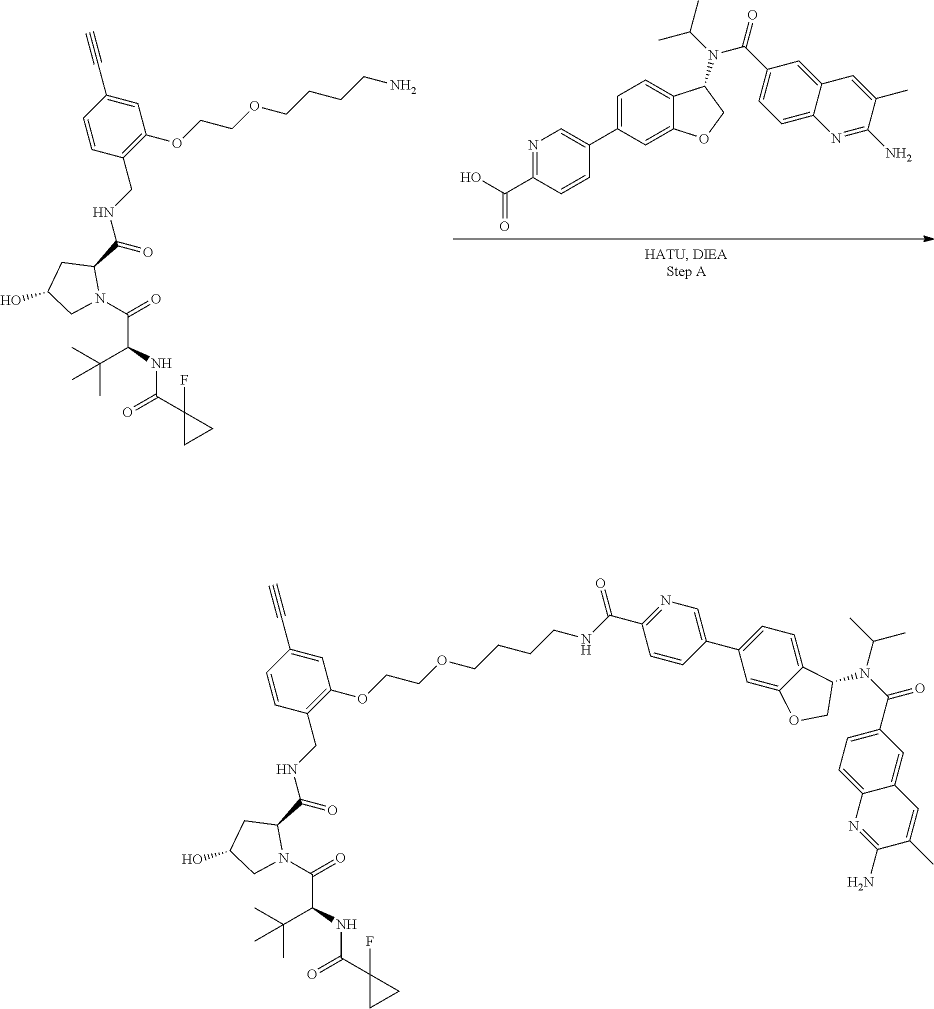 [SMILES: C#Cc1ccc(CNC(=O)[C@@H]2C[C@@H](O)CN2C(=O)[C@@H](NC(=O)C2(F)CC2)C(C)(C)C)c(OCCOCCCCN)c1 ; Cc1cc2cc(C(=O)N(C(C)C)[C@@H]3COc4cc(-c5ccc(C(=O)O)nc5)ccc43)ccc2nc1N ; C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C(=O)c3ccc4nc(N)c(C)cc4c3)C(C)C)cn2)c1]

Example A86 was synthesized following the same procedures as described in Example A85 (3.5 mg, 5% yield) as a white solid. LC-MS (ESI): mass calcd, for C₅₈H₆₇FN₈O₉, 1038.50; m/z found, 1139.6 [M+H]⁺. ¹H NMR (400 MHz, DMSO-d₆) δ 13.97 (s, 1H), 8.88 (s, 1H), 8.79 (t, J=6.1 Hz, 1H), 8.65 (s, 2H), 8.46 (t, J=6.0 Hz, 1H), 8.30 (s, 1H), 8.25-8.16 (m, 1H), 8.07 (d, J=8.2 Hz, 1H), 7.88 (s, 1H), 7.72 (s, 2H), 7.37 (s, 1H), 7.30-7.26 (m, 2H), 7.25-7.18 (m, 1H), 7.05 (s, 1H), 6.96 (d, J=7.7 Hz, 1H), 5.49-5.39 (m, 1H), 4.82-4.72 (m, 1H), 4.70-4.65 (m, 1H), 4.58 (d, J=9.2 Hz, 1H), 4.48 (t, J=8.2 Hz, 1H), 4.33 (s, 1H), 4.25 (d, J=6.3 Hz, 1H), 4.18 (d, J=5.7 Hz, 1H), 4.16-4.13 (m, 2H), 4.11 (s, 1H), 3.64 (d, J=3.7 Hz, 2H), 3.62-3.59 (m, 3H), 3.51 (t, J=5.9 Hz, 3H), 3.35-3.30 (m, 2H), 2.31 (s, 3H), 2.12-2.03 (m, 1H), 1.92-1.84 (m, 1H), 1.65-1.49 (m, 5H), 1.44-1.33 (m, 5H), 1.24-1.16 (m, 4H), 0.94 (s, 9H).

### Example A87. 2-amino-N—((S)-6-(6-((4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)carbamoyl)pyridin-3-yl)-2,3-dihydrobenzofuran-3-yl)-N-isopropyl-3-methylquinoline-6-carboxamide

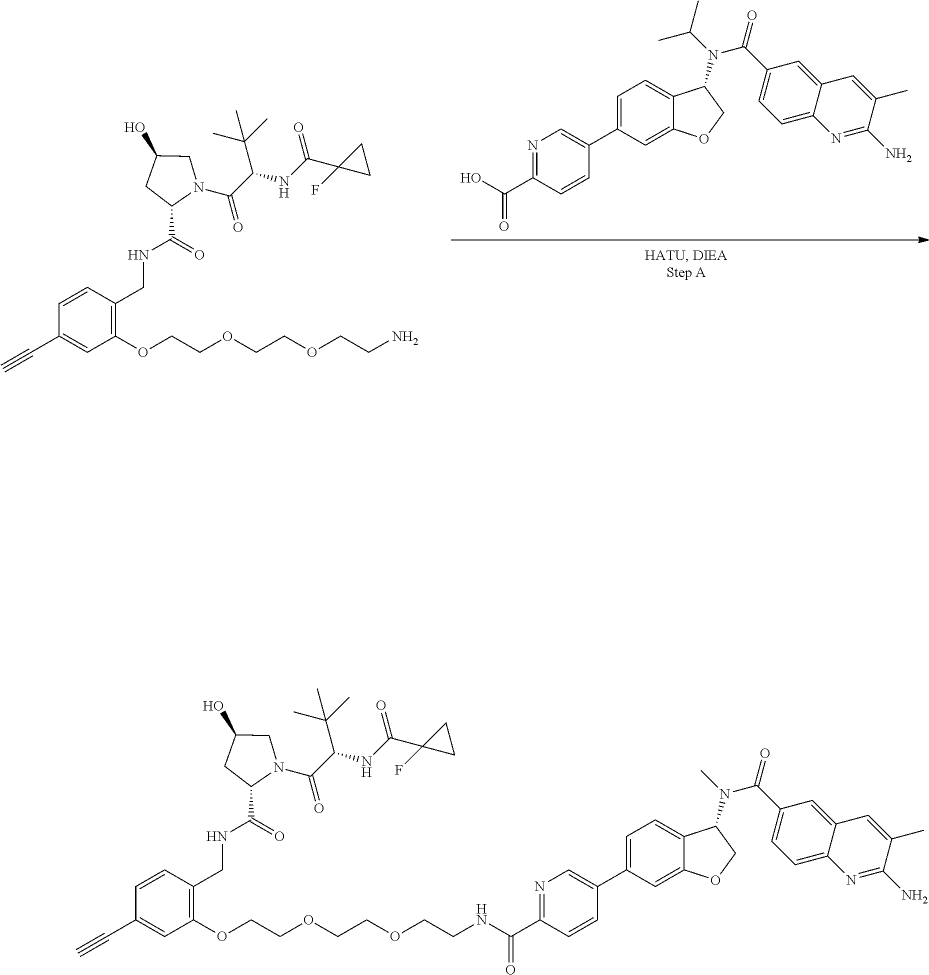 [SMILES: C#Cc1ccc(CNC(=O)[C@@H]2C[C@@H](O)CN2C(=O)[C@@H](NC(=O)C2(F)CC2)C(C)(C)C)c(OCCOCCOCCN)c1 ; Cc1cc2cc(C(=O)N(C(C)C)[C@@H]3COc4cc(-c5ccc(C(=O)O)nc5)ccc43)ccc2nc1N ; C#Cc1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c(C)cc4c3)cn2)c1]

Example A87 was synthesized following the same procedures as described in Example A85 (1.8 mg, 3% yield) as a white solid. LC-MS (ESI): mass calcd, for C₅₈H₆₇FN₈O₁₀, 1054.50; m/z found, 1155.7 [M+H]⁺. ¹H NMR (400 MHz, DMSO-d₆) δ 8.89 (s, 1H), 8.69 (t, J=5.8 Hz, 1H), 8.56-8.44 (m, 2H), 8.38-8.14 (m, 2H), 8.08 (d, J=8.1 Hz, 1H), 7.98-7.81 (m, 1H), 7.79-7.64 (m, 2H), 7.49-7.33 (m, 1H), 7.33-7.24 (m, 3H), 7.23-7.15 (m, 1H), 7.03 (s, 1H), 6.96 (d, J=8.0 Hz, 1H), 5.42 (s, 1H), 4.89-4.62 (m, 2H), 4.58 (d, J=9.1 Hz, 1H), 4.48 (t, J=8.2 Hz, 1H), 4.33 (s, 1H), 4.29-4.23 (m, 1H), 4.18 (d, J=5.6 Hz, 1H), 4.15-4.09 (m, 3H), 3.80-3.75 (m, 2H), 3.70-3.60 (m, 4H), 3.60-3.56 (m, 5H), 3.50-3.48 (m, 2H), 2.34-2.29 (m, 3H), 2.07-2.03 (m, 1H), 1.91-1.87 (m, 1H), 1.40-1.32 (m, 4H), 1.25-1.18 (m, 6H), 0.94 (s, 9H).

### Example A88. 2-amino-N—((S)-6-(6-(((18,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methyl-3-(tetrahydro-2H-pyran-4-yl)quinoline-6-carboxamide

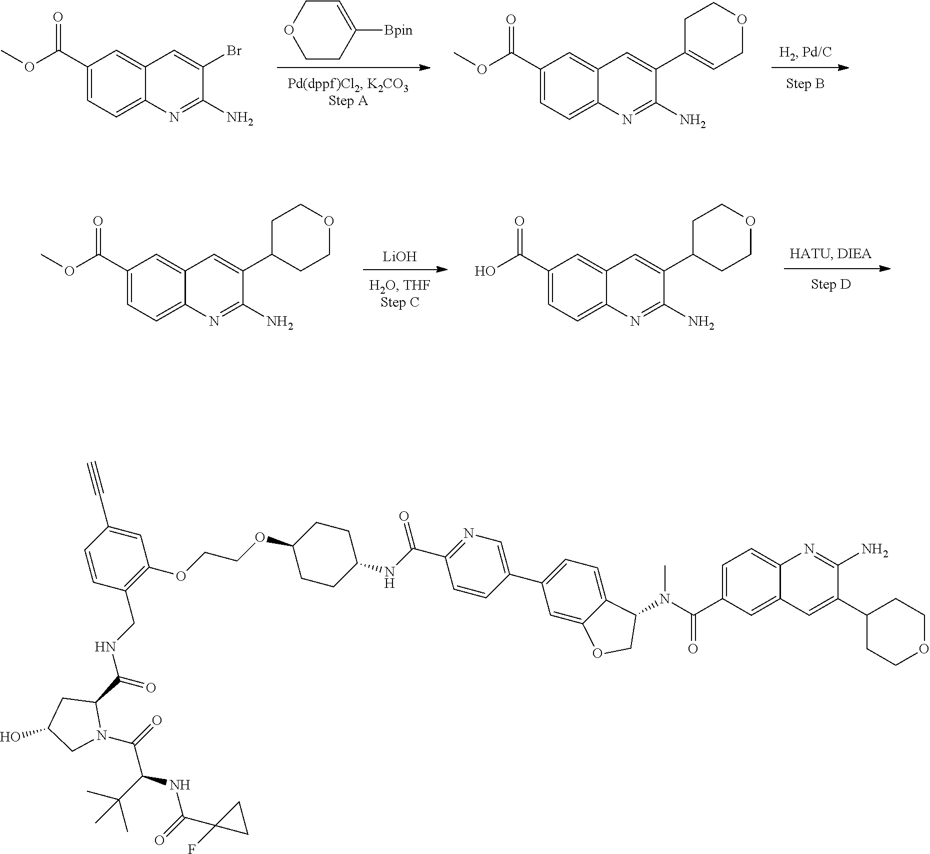

Step A: methyl 2-amino-3-(3,6-dihydro-2H-pyran-4-yl)quinoline-6-carboxylate

To a solution of methyl 2-amino-3-bromoquinoline-6-carboxylate (100 mg, 356 μmol, 1.0 eq.), 2-(3,6-dihydro-2H-pyran-4-yl)-4,4,5,5-tetramethyl-1,3,2-dioxaborolane (112 mg, 534 μmol, 1.5 eq.) and K₂CO₃ (147 mg, 1.07 mmol, 3.0 eq.) in 1,4-Dioxane (4 mL) and Water (0.2 mL) was added Pd(dppf)Cl₂ (23 mg, 35.6 μmol, 0.1 eq.) at room temperature. The mixture was stirred at 100° C. for 16 hours. The mixture was filtered and concentrated under reduced pressure. The residue was purified by flash column chromatography on silica gel (100-200 mesh silica gel, 0-100% EtOAc in PE) to give methyl 2-amino-3-(3,6-dihydro-2H-pyran-4-yl)quinoline-6-carboxylate (70 mg, 69% yield) as a yellow solid.

LC-MS (ESI): mass calcd, for C₁₆H₁₆N₂O₃, 284.12; m/z found, 285.1 [M+H]⁺.

Step B: methyl 2-amino-3-(tetrahydro-2H-pyran-4-yl)quinoline-6-carboxylate

To a solution of methyl 2-amino-3-(3,6-dihydro-2H-pyran-4-yl)quinoline-6-carboxylate (70 mg, 246 μmol, 1.0 eq.) in MeOH (4 mL) was added Pd/C(10 mg, 94.0 μmol, 0.4 eq.). The mixture was stirred at room temperature for 16 hours under H₂ (25 Psi). The reaction mixture was filtered and concentrated under reduced pressure to give a residue, which was triturated with MeOH, filtered and dried over to afford methyl 2-amino-3-(tetrahydro-2H-pyran-4-yl)quinoline-6-carboxylate (50 mg, 71% yield) as a colorless oil, which was used directly without further purification.

LC-MS (ESI): mass calcd, for C₁₆H₁₈N₂O₃, 286.13; m/z found, 287.1 [M+H]⁺.

Step C: methyl 2-amino-3-(tetrahydro-2H-pyran-4-yl)quinoline-6-carboxylate

A solution of methyl 2-amino-3-(tetrahydro-2H-pyran-4-yl)quinoline-6-carboxylate (30 mg, 105 μmol, 1.0 eq.) and LiOH (4 mg, 157 μmol, 1.5 eq.) in THF (2 mL) and Water (1 mL) was stirred at room temperature for 8 hours. The residue was concentrated under reduced pressure to give 2-amino-3-(tetrahydro-2H-pyran-4-yl)quinoline-6-carboxylic acid (25 mg, 88% yield) as a white solid.

LC-MS (ESI): mass calcd, for C₁₅H₁₆N₂O₃, 272.12; m/z found, 273.1 [M+H]⁺.

### Step D

Example A88 was synthesized following the same procedures as described in Example A71 (4 mg, 2% yield) as a white solid. LC-MS (ESI): mass calcd, for C₆₂H₇₁FN₈O₁₀, 1106.53; m/z found, 1107.4 [M+H]⁺. ¹H NMR (400 MHz, DMSO-₆) δ 8.92 (s, 1H), 8.73 (s, 2H), 8.51-8.39 (m, 3H), 8.30-8.25 (m, 1H), 8.14-8.07 (m, 2H), 7.90-7.86 (m, 1H), 7.81-7.72 (m, 1H), 7.58-7.54 (m, 1H), 7.42-7.26 (m, 4H), 7.06 (s, 1H), 6.98 (d, J=8.2 Hz, 1H), 4.87-4.65 (m, 2H), 4.59 (d, J=9.3 Hz, 1H), 4.54-4.45 (m, 1H), 4.41-4.32 (m, 1H), 4.31-4.17 (m, 2H), 4.15-4.10 (m, 3H), 4.03-3.97 (m, 2H), 3.85-3.76 (m, 3H), 3.67-3.52 (m, 5H), 3.10-3.05 (m, 1H), 2.67 (s, 3H), 2.10-2.01 (m, 3H), 1.95-1.67 (m, 8H), 1.55-1.46 (m, 2H), 1.40-1.37 (m, 1H), 1.35-1.19 (m, 6H), 0.95 (s, 9H).

### Example A89. 2-amino-3-cyclopropyl-N—((S)-6-(6-((2-(2-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)ethoxy)ethyl)carbamoyl)pyridin-3-yl)-2,3-dihydrobenzofuran-3-yl)-N-methylquinoline-6-carboxamide

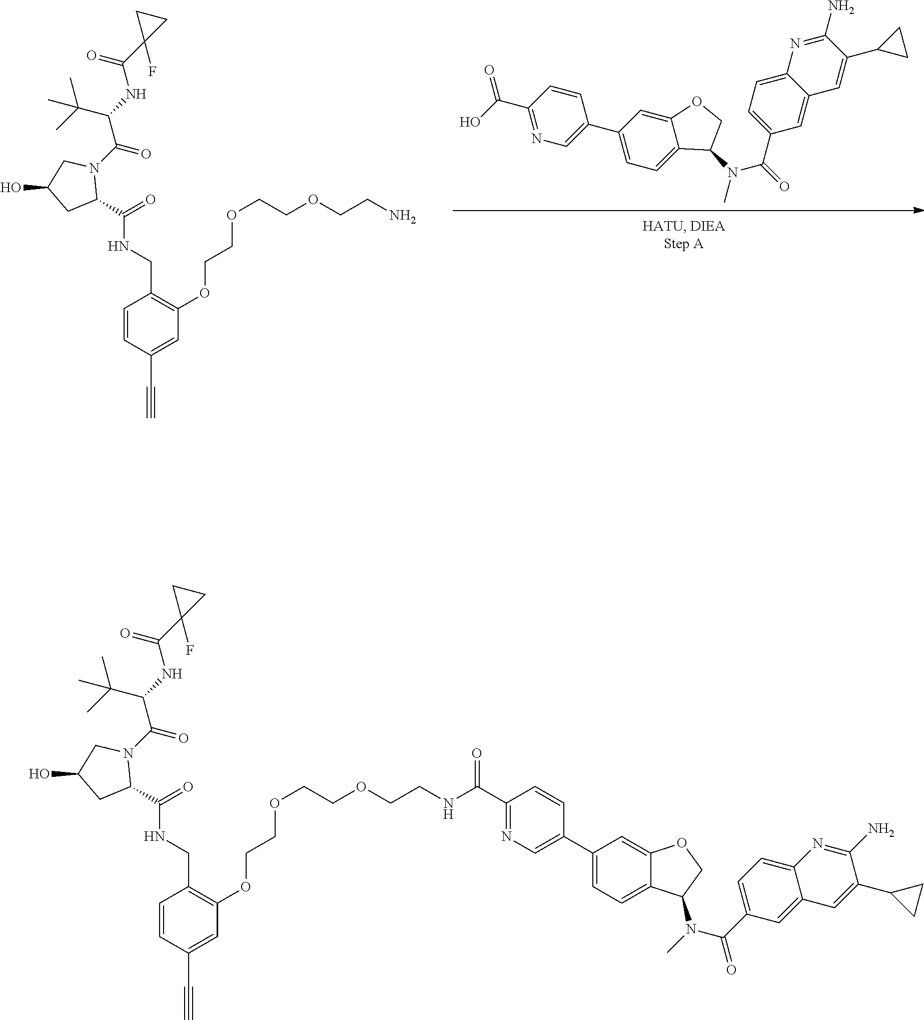 [SMILES: C#Cc1ccc(CNC(=O)[C@@H]2C[C@@H](O)CN2C(=O)[C@@H](NC(=O)C2(F)CC2)C(C)(C)C)c(OCCOCCOCCN)c1 ; CN(C(=O)c1ccc2nc(N)c(C3CC3)cc2c1)[C@@H]1COc2cc(-c3ccc(C(=O)O)nc3)ccc21 ; C#Cc1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3ccc4nc(N)c(C5CC5)cc4c3)cn2)c1]

Example A89 was synthesized following the same procedures as described in Example A85 (3 mg, 5% yield) as a white solid. LC-MS (ESI): mass calcd, for C₅₈H₆₅FN₈O₁₀, 1052.5; m/z found, 1053.5 [M+H]⁺. ¹H NMR (400 MHz, DMSO-d₆) δ 8.92 (s, 1H), 8.79 (s, 1H), 8.71 (t, J=5.7 Hz, 1H), 8.47 (t, J=6.0 Hz, 1H), 8.27 (dd, J=8.2 Hz, 1H), 8.21 (s, 1H), 8.09 (d, J=8.2 Hz, 1H), 8.04 (s, 1H), 7.86 (d, J=8.5 Hz, 1H), 7.80-7.72 (m, 1H), 7.54 (d, J=7.7 Hz, 1H), 7.39 (d, J=8.2 Hz, 1H), 7.35 (s, 1H), 7.32-7.25 (m, 2H), 7.03 (s, 1H), 6.96 (d, J=7.8 Hz, 1H), 4.84-4.66 (m, 2H), 4.58 (d, J=9.2 Hz, 1H), 4.49 (t, J=8.3 Hz, 1H), 4.33 (s, 1H), 4.29-4.23 (m, 1H), 4.22-4.17 (m, 1H), 4.15-4.10 (m, 3H), 3.80-3.76 (m, 2H), 3.71-3.66 (m, 1H), 3.65-3.58 (m, 8H), 3.51-3.49 (m, 2H), 2.66 (s, 3H), 2.10-2.04 (m, 1H), 1.94-1.86 (m, 2H), 1.40-1.32 (m, 2H), 1.24-1.20 (m, 2H), 1.11-1.06 (m, 2H), 0.95 (s, 9H), 0.84-0.78 (m, 2H).

### Example A90. 2-amino-7-fluoro-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-3-(hydroxymethyl)-N,4-dimethylquinoline-6-carboxamide

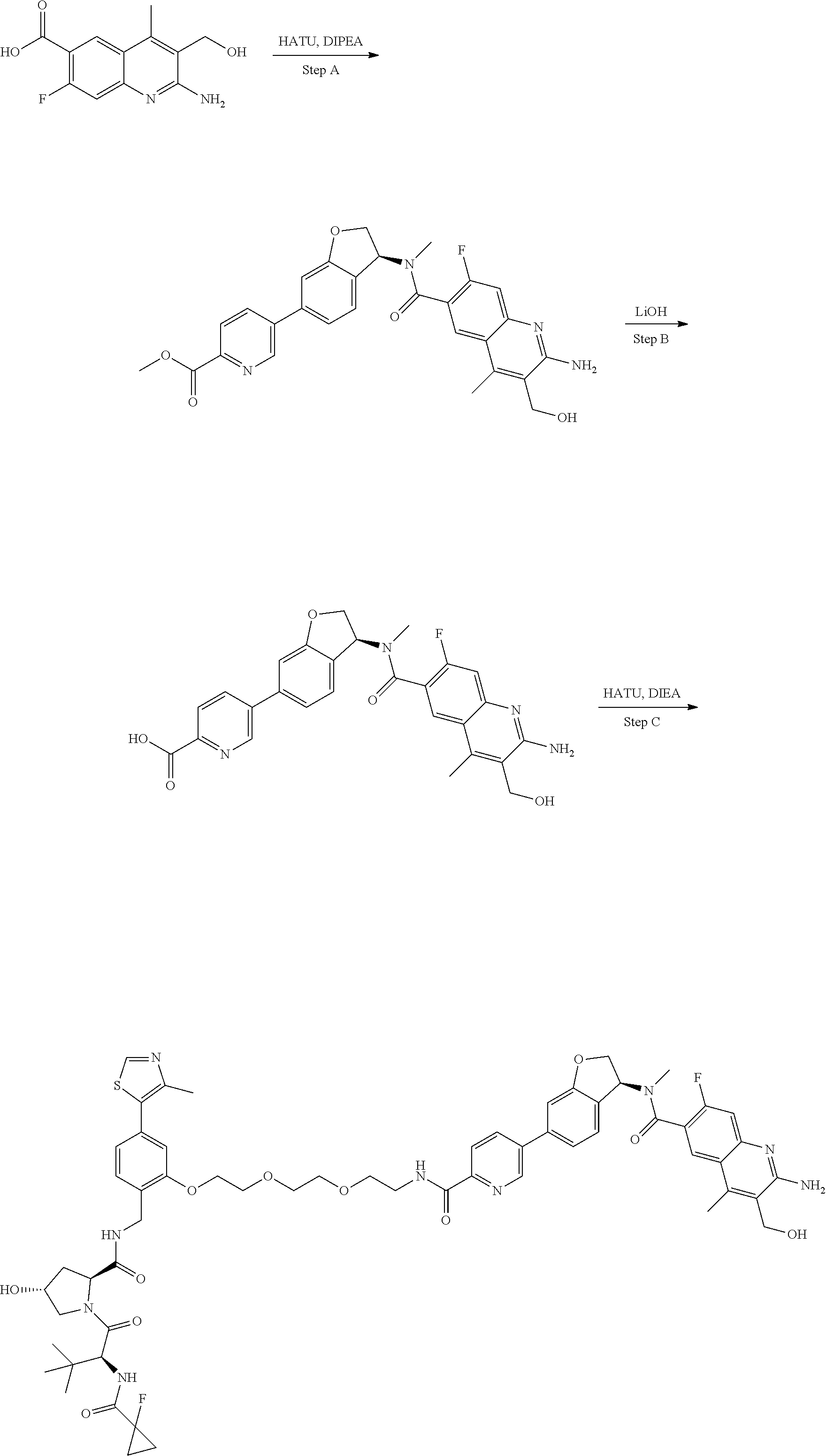 [SMILES: Cc1c(CO)c(N)nc2cc(F)c(C(=O)O)cc12 ; COC(=O)c1ccc(-c2ccc3c(c2)OC[C@H]3N(C)C(=O)c2cc3c(C)c(CO)c(N)nc3cc2F)cn1 ; "LiOH" ; Cc1c(CO)c(N)nc2cc(F)c(C(=O)N(C)[C@@H]3COc4cc(-c5ccc(C(=O)O)nc5)ccc43)cc12 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCNC(=O)c2ccc(-c3ccc4c(c3)OC[C@H]4N(C)C(=O)c3cc4c(C)c(CO)c(N)nc4cc3F)cn2)c1]

Step A: methyl(S)-5-(3-(2-amino-7-fluoro-3-(hydroxymethyl)-N,4-dimethylquinoline-6-carboxamido)-2,3-dihydrobenzofuran-6-yl)picolinate

To a stirred mixture of 2-amino-7-fluoro-3-(hydroxymethyl)-4-methylquinoline-6-carboxylic acid (25 mg, 99.9 μmol, 1.0 eq. WO2024140923) in DMF (5 mL) were added methyl(S) -5-(3-(methylamino)-2,3-dihydrobenzofuran-6-yl)picolinate (28 mg, 99.9 μmol, 1.0 eq.) and HATU (76 mg, 200 μmol, 2.0 eq.) and DIEA (64.6 mg, 87.0 μL, 500 μmol, 5.0 eq.) at room temperature. The mixture was stirred at room temperature for 2 hours. The resulting solution was diluted with 100 mL of water and extracted with EtOAc (30 mL×3). The combined organic layers were washed with brine, dried over anhydrous Na₂SO₄ and concentrated under reduced pressure. The residue was purified by prep-HPLC(C18, 20%-95% MeCN in water with 0.1% FA) to give methyl(S)-5-(3-(2-amino-7-fluoro-3-(hydroxymethyl)-N,4-dimethylquinoline-6-carboxamido)-2,3-dihydrobenzofuran-6-yl)picolinate (30 mg, 58% yield) as a white solid.

LC-MS (ESI): mass calcd, for C₂₈H₂₅FN₄O₅, 516.18; m/z found, 517.1 [M+H]⁺.

Step B: (S)-5-(3-(2-amino-7-fluoro-3-(hydroxymethyl)-N,4-dimethylquinoline-6-carboxamido)-2,3-dihydrobenzofuran-6-yl)picolinic acid

To a solution of methyl(S)-5-(3-(2-amino-7-fluoro-3-(hydroxymethyl)-N,4-dimethylquinoline-6-carboxamido)-2,3-dihydrobenzofuran-6-yl)picolinate (15 mg, 29.0 μmol, 1. eq.) in THF (2.00 mL) and Water (1.00 mL) was added LiOH (1 mg, 43.6 μmol, 1.5 eq.) at room temperature. The mixture was stirred at room temperature for 2 hours. The residue was concentrated under reduced pressure to give(S)-5-(3-(2-amino-7-fluoro-3-(hydroxymethyl)-N,4-dimethylquinoline-6-carboxamido)-2,3-dihydrobenzofuran-6-yl)picolinic acid (14.0 mg, 96% yield) as a white solid.

LC-MS (ESI): mass calcd, for C₂₇H₂₃FN₄O₅, 502.17; m/z found, 503.1 [M+H]⁺.

Step C: 2-amino-7-fluoro-N—((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-3-(hydroxymethyl)-N,4-dimethylquinoline-6-carboxamide

To a stirred mixture of (2S,4R)—N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20 mg, 30.1 μmol, 1.0 eq.) in DMF (3 mL) were added(S)-5-(3-(2-amino-7-fluoro-3-(hydroxymethyl)-N,4-dimethylquinoline-6-carboxamido)-2,3-dihydrobenzofuran-6-yl)picolinic acid (15 mg, 30.1 μmol, 1.0 eq.), HATU (23 mg, 60.3 μmol, 2.0 eq.) and DIEA (19 mg, 26.2 μL, 151 μmol, 5.0 eq.), the mixture was stirred at room temperature for 2 hours. The resulting solution was diluted with 100 mL of water and extracted with EtOAc (30 mL×3). The combined organic layers were washed with brine, dried over anhydrous Na₂SO₄ and concentrated under reduced pressure. The residue was purified by prep-HPLC(C18, 20%-95% MeCN in water with 0.1% FA) to give 2-amino-7-fluoro-N—((S)-6-(6-((2-(2-(2-(2-(((2S,4R)-1-((S)-2-(1-fluorocyclopropane-1-carboxamido)-3,3-dimethyl butanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-3-(hydroxymethyl)-N,4-dimethylquinoline-6-carboxamide (7 mg, 20% yield) as a white solid.

LC-MS (ESI): mass calcd, for C₅₉H₆₇F₂N₉O₁₁S, 1147.46; m/z found, 1148.4 [M+H]⁺. 1H NMR (400 MHz, DMSO-d₆) δ 8.97-8.94 (m, 1H), 8.93-8.89 (m, 1H), 8.72-8.68 (m, 1H), 8.61-8.46 (m, 3H), 8.29-8.05 (m, 3H), 7.64-7.47 (m, 2H), 7.40-7.27 (m, 4H), 7.01 (s, 1H), 6.95 (d, J=7.8 Hz, 1H), 6.49-5.50 (m, 1H), 4.87-4.67 (m, 4H), 4.59 (d, J=9.5 Hz, 1H), 4.53-4.49 (m, 1H), 4.35-4.15 (m, 6H), 3.82-3.78 (m, 4H), 3.59-3.56 (m, 4H), 3.52-3.46 (m, 4H), 2.78-2.70 (m, 5H), 2.56 (s, 2H), 2.44-2.42 (m, 3H), 2.11-2.05 (m, 1H), 1.95-1.88 (m, 1H), 1.40-1.32 (m, 2H), 1.23-1.19 (m, 2H), 0.95 (s, 9H).

### Example A91. 2-amino-3-cyano-N-((S)-6-(6-(((1S,4S)-4-(2-(5-ethynyl-2-(((28,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

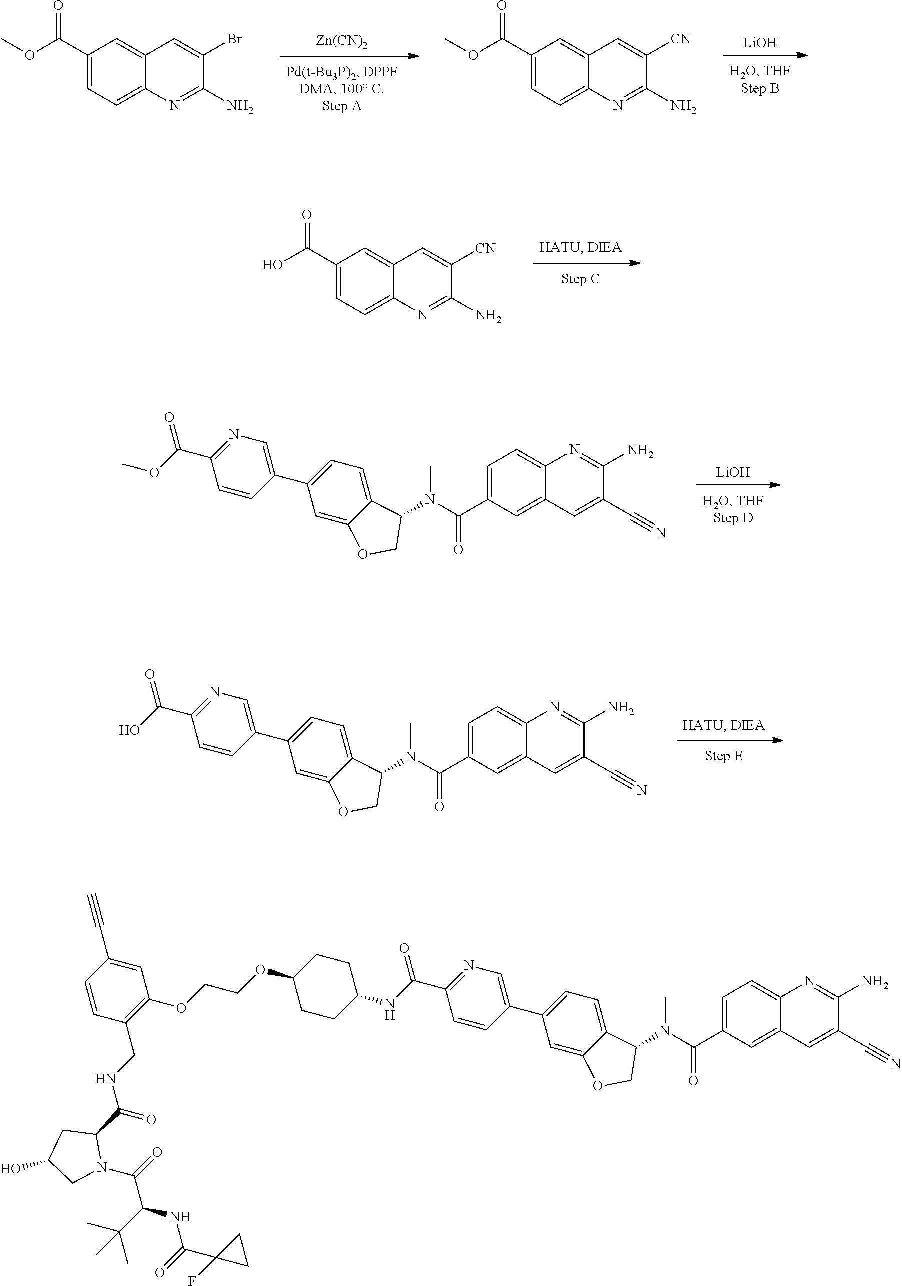

Step A: methyl 2-amino-3-cyanoquinoline-6-carboxylate

To a solution of methyl 2-amino-3-bromoquinoline-6-carboxylate (100 mg, 356 μmol, 1.0 eq.), dppf (20 mg, 35.6 μmol, 0.1 eq.) and Zn(CN)₂ (84 mg, 711 μmol, 2.0 eq.) in DMA (5 mL) was added Pd(t-Bu₃P)₂ (18 mg, 35.6 μmol, 0.1 eq.). The mixture was stirred at 150° C. for 1 hour in microwave. The resulting solution was diluted with 10 mL of water and extracted with EtOAc (10 mL×3). The combined organic layers were washed with brine (20 mL), dried over Na₂SO₄ and concentrated under reduced pressure. The residue was purified by flash column chromatography on silica gel (100-200 mesh silica gel, 0-20% EtOAc in PE) to give methyl 2-amino-3-cyanoquinoline-6-carboxylate (60 mg, 67% yield) as a yellow solid. LC-MS (ESI): mass calcd, for C12H9N302, 227.07; m/z found, 228.3 [M+H]⁺.

Step B: 2-amino-3-cyanoquinoline-6-carboxylic acid

To a solution of methyl 2-amino-3-cyanoquinoline-6-carboxylate (60 mg, 264 μmol, 1.0 eq.) in THF (2.0 mL) and Water (2.0 mL) was added LiOH (22 mg, 528 μmol, 2.0 eq.). The mixture was stirred at room temperature for 1 hour. The resulting solution was concentrated under reduced pressure to give 2-amino-3-cyanoquinoline-6-carboxylic acid (56 mg, 99% yield) as a yellow solid.

LC-MS (ESI): mass calcd, for C₁₁H₇N₃O₂, 213.05; m/z found, 213.9 [M+H]⁺.

### Steps C-E

Example A91 was synthesized following the same procedures as described in Example A71 (5.6 mg, 3% yield over 3 steps) as a white solid. LC-MS (ESI): mass calcd, for C₅₈H₆₂FN₉O₉, 1147.47; m/z found, 1048.3 [M+H]⁺. ¹H NMR (400 MHz, DMSO-d₆) δ 8.93 (s, 1H), 8.83 (s, 1H), 8.53-8.45 (m, 2H), 8.28 (dd, J=8.2, 2.0 Hz, 1H), 8.09 (d, J=8.2 Hz, 1H), 7.98 (s, 1H), 7.83 (d, J=8.7 Hz, 1H), 7.63-7.55 (m, 2H), 7.54-7.36 (m, 3H), 7.36-7.28 (m, 3H), 7.07 (s, 1H), 6.98 (d, J=8.3 Hz, 1H), 4.80-4.67 (m, 2H), 4.60 (d, J=9.1 Hz, 1H), 4.50 (t, J=8.2 Hz, 1H), 4.36 (s, 1H), 4.30-4.20 (m, 2H), 4.19-4.08 (m, 4H), 3.82-3.77 (m, 4H), 3.39-3.31 (m, 2H), 2.68 (s, 3H), 2.11-2.02 (m, 3H), 1,96-1.90 (m, 1H), 1.89-1.83 (m, 2H), 1.55-1.45 (m, 2H), 1.40-1.28 (m, 4H), 1.24-1.19 (m, 2H), 0.96 (s, 9H).

### Example A92. 2-amino-3-cyclobutyl-N-((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

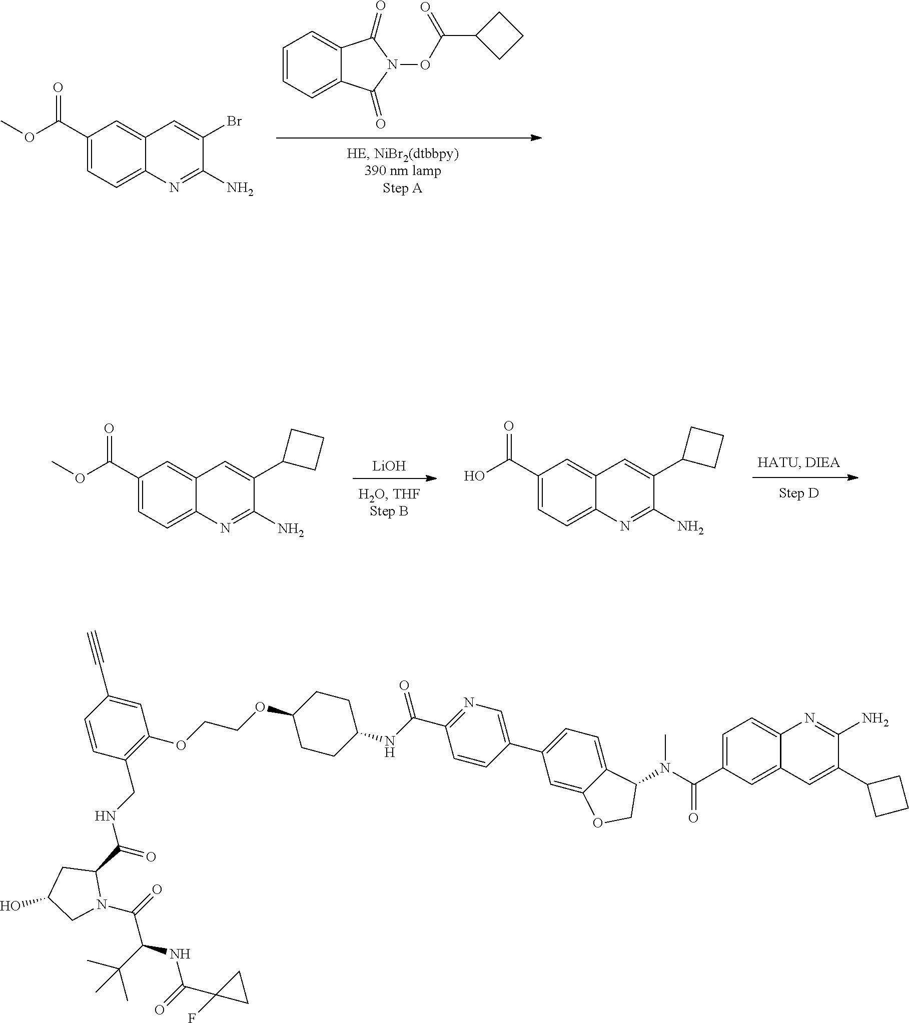 [SMILES: COC(=O)c1ccc2nc(N)c(Br)cc2c1 ; O=C(ON1C(=O)c2ccccc2C1=O)C1CCC1 ; COC(=O)c1ccc2nc(N)c(C3CCC3)cc2c1 ; "LiOH" ; Nc1nc2ccc(C(=O)O)cc2cc1C1CCC1 ; C#Cc1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(C6CCC6)cc5c4)cn3)CC2)c1]

Step A: methyl 2-amino-3-cyclobutylquinoline-6-carboxylate

To a solution of methyl 2-amino-3-bromoquinoline-6-carboxylate (200 mg, 711 μmol, 1.0 eq.), Ni(dtbbpy) Br₂ (35 mg, 71.1 μmol, 0.1 eq.), HE (180 mg, 711 μmol, 1.0 eq.) in DMA (8.0 mL) was added 1,3-dioxoisoindolin-2-yl cyclobutanecarboxylate (245 mg, 1.07 mmol, 1.5 eq.), followed by Sodium bicarbonate (120 mg, 1.42 mmol, 2.0 eq.). The mixture was stirred under 390 nm purple LED at room temperature for 8 hours. The resulting solution was diluted with 50 mL of water and extracted with EtOAc (20 mL×3). The combined organic layers were washed with brine 50 mL), dried over Na₂SO₄ and concentrated under reduced pressure. The residue was purified by flash column chromatography on silica gel (100-200 mesh silica gel, 0-45% EtOAc in PE) and prep-HPLC to give methyl 2-amino-3-cyclobutylquinoline-6-carboxylate (12 mg, 7% yield) as a white solid.

¹H NMR (400 MHz, DMSO-d₆) δ 8.62 (s, 1H), 8.45 (s, 1H), 8.41-8.25 (m, 1H), 8.19 (d, J=8.8 Hz, 1H), 7.74 (d, J=8.7 Hz, 1H), 3.91 (s, 3H), 3.63-3.56 (m, 1H), 2.48-2.42 (m, 2H), 2.22-2.11 (m, 2H), 2.08-1.98 (m, 1H), 1.88 (t, J=9.3 Hz, 1H).

Step B: 2-amino-3-cyclobutylquinoline-6-carboxylic acid

To a solution of methyl 2-amino-3-cyclobutylquinoline-6-carboxylate (13 mg, 50.7 μmol, 1.0 eq.) in THF (1.0 mL), MeOH (0.5 mL) and Water (1.0 mL) was added LiOH (4 mg, 101 μmol, 2.0 eq.) and stirred at room temperature for 16 hours. The resulting solution was concentrated under reduced pressure to give 2-amino-3-cyclobutylquinoline-6-carboxylic acid (12 mg, 98% yield) as a yellow solid.

LC-MS (ESI): mass calcd, for C₁₄H₁₄N₂O₂, 242.11; m/z found, 242.9 [M+H]⁺.

Step C: 2-amino-3-cyclobutyl-N-((S)-6-(6-(((1S,4S)-4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

Example A92 was synthesized following the same procedures as described in Example A71 (5.3 mg, 12% yield) as a white solid. LC-MS (ESI): mass calcd, for C₆₁H₆₉FN₈O₉, 1176.52; m/z found, 1077.8 [M+H]⁺. ¹H NMR (400 MHz, DMSO-d₆) δ 8.93 (s, 1H), 8.58-8.38 (m, 4H), 8.28 (d, J=8.0 Hz, 1H), 8.16 (s, 1H), 8.09 (d, J=8.2 Hz, 1H), 7.88 (d, J=8.7 Hz, 1H), 7.83-7.70 (m, 1H), 7.57 (d, J=7.7 Hz, 1H), 7.46-7.25 (m, 4H), 7.07 (s, 1H), 6.98 (d, J=7.8 Hz, 1H), 4.83 (s, 1H), 4.72-4.67 (m, 1H), 4.60 (d, J=9.2 Hz, 1H), 4.50 (t, J=8.2 Hz, 1H), 4.36 (s, 1H), 4.31-4.19 (m, 2H), 4.15-4.12 (m, 2H), 3.91-3.76 (m, 4H), 3.66-3.59 (m, 4H), 2.68 (s, 3H), 2.27-1.96 (m, 7H), 1.95-1.80 (m, 4H), 1.56-1.47 (m, 2H), 1.41-1.28 (m, 4H), 1.26-1.19 (m, 4H), 0.96 (s, 9H)

### Example A93. N1-(6-amino-5-methylpyridin-3-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-N2-isopropyloxalamide

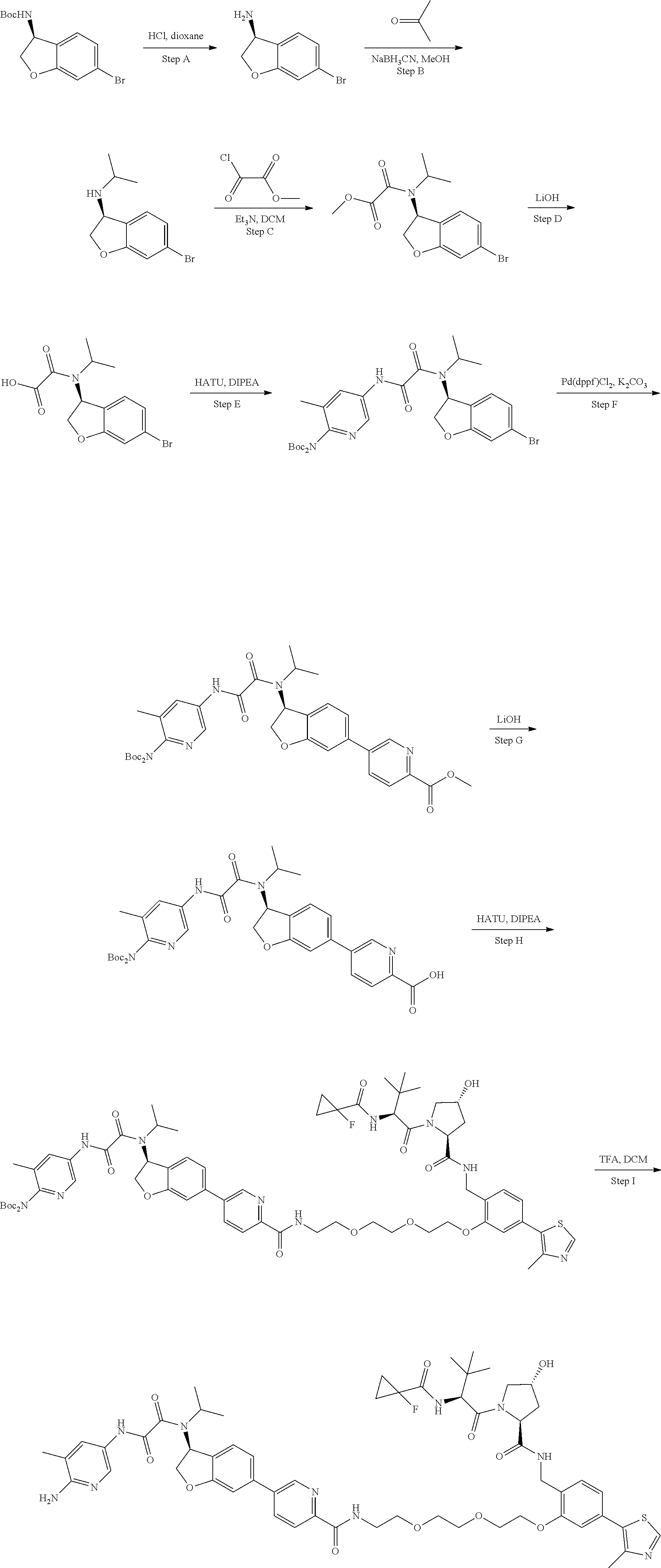

Step A: (S)-6-bromo-2,3-dihydrobenzofuran-3-amine

To a solution of tert-butyl(S)-(6-bromo-2,3-dihydrobenzofuran-3-yl)carbamate (1.2 g, 3.82 mmol, 1.0 eq.) in DCM (5.0 mL) was added HCl-dioxane (5.0 mL) and stirred at room temperature for 3 hours. The resulting solution was concentrated under reduced pressure to give(S)-6-bromo-2,3-dihydrobenzofuran-3-amine (810 mg, 99% yield) as a yellow solid.

LC-MS (ESI): mass calcd, for C₈H₈BrNO, 212.98; m/z found, 199.1 [M-17+H]⁺.

Step B: (S)-6-bromo-N-isopropyl-2,3-dihydrobenzofuran-3-amine

To a solution of(S)-6-bromo-2,3-dihydrobenzofuran-3-amine (810 mg, 3.78 mmol, 1.0 eq.) in DCE (15.0 mL) was added TEA (527 μL, 3.78 mmol, 1.0 eq.) at room temperature. The mixture was acidified with AcOH (217 μL, 3.78 mmol, 1.0 eq.) till pH=3. Then the acetone (5.0 mL) was added and stirred at room temperature for 2 hours under N₂. NaBH (OAc) 3 (1.6 g, 7.57 mmol, 2.0 eq.) was added and the mixture was stirred at room temperature for 1 hour. The resulting solution was diluted with 50 mL of water and extracted with DCM/i-PrOH=10/1 (30 mL×3). The combined organic layers were washed with brine (50 mL), dried over Na₂SO₄ and concentrated under reduced pressure to give(S)-6-bromo-N-isopropyl-2,3-dihydrobenzofuran-3-amine (800 mg, 83% yield) which was used in the next step without further purification.

LC-MS (ESI): mass calcd, for C₁₁H₁₄BrNO, 255.03; m/z found, 255.6 [M+H]⁺.

Step C: methyl(S)-2-((6-bromo-2,3-dihydrobenzofuran-3-yl)(isopropyl)amino)-2-oxoacetate

To a solution of(S)-6-bromo-N-isopropyl-2,3-dihydrobenzofuran-3-amine (100 mg, 390 μmol, 1.0 eq.), TEA (54 μL, 390 μmol, 1.0 eq.) and methyl 2-chloro-2-oxoacetate (72 mg, 586 μmol, 1.5 eq.) in DCM (7.0 mL) was stirred at 0° C. for 40 min. The resulting solution was diluted with 20 mL of water and extracted with DCM (10 mL×3). The combined organic layers were washed with brine (20 mL), dried over Na₂SO₄ and concentrated under reduced pressure. The residue was purified by flash column chromatography on silica gel (100-200 mesh silica gel, 0-25% EtOAc in PE) to give methyl(S)-2-((6-bromo-2,3-dihydrobenzofuran-3-yl)(isopropyl)amino)-2-oxoacetate (67.0 mg, 50.2% yield) as a yellow oil.

¹H NMR (400 MHz, DMSO-d₆) δ 7.33-6.98 (m, 3H), 5.52-5.31 (m, 1H), 4.71 (q, J=10.0 Hz, 1H), 4.59-4.37 (m, 1H), 3.75 (s, 3H), 1.37-1.19 (m, 6H).

Step D: (S)-2-((6-bromo-2,3-dihydrobenzofuran-3-yl)(isopropyl)amino)-2-oxoacetic acid

To a solution of methyl(S)-2-((6-bromo-2,3-dihydrobenzofuran-3-yl)(isopropyl)amino)-2-oxoacetate (57 mg, 167 μmol, 1.0 eq.) in EtOH (4.0 mL) and Water (3.0 mL) was added a solution of LiOH (8 mg, 183 μmol, 1.1 eq.) in Water (1.0 mL) at 0~5° C. The resulting mixture was stirred at 0~5° C. for 1 hour. The resulting solution was adjusted at pH=2~3 with 1 M aq. HCl. The suspension was filtered and the filter cake was washed with water, dried in vacuum to give(S)-2-((6-bromo-2,3-dihydrobenzofuran-3-yl)(isopropyl)amino)-2-oxoacetic acid (54 mg, 98% yield) as a white solid.

LC-MS (ESI): mass calcd, for C₁₃H₁₄BrNO₄, 327.01; m/z found, 326.1 [M−H]⁺.

Step E: tert-butyl(S)-(5-(2-((6-bromo-2,3-dihydrobenzofuran-3-yl)(isopropyl)amino)-2-oxoacetamido)-3-methylpyridin-2-yl)(tert-butoxycarbonyl)carbamate

To a solution of(S)-2-((6-bromo-2,3-dihydrobenzofuran-3-yl)(isopropyl)amino)-2-oxoacetic acid (15 mg, 45.7 μmol, 1.0 eq.), tert-butyl (5-amino-3-methylpyridin-2-yl)(tert-butoxycarbonyl)carbamate (15 mg, 45.7 μmol, 1.0 eq.) and HATU (19 mg, 50.3 μmol, 1.1 eq.) in DMF (3 mL) was added DIPEA (24 μL, 137 μmol, 3.0 eq.) at 0° C. The mixture was stirred at room temperature for 30 min. The resulting solution was diluted with 10 mL of water and extracted with DCM (10 mL×3). The combined organic layers were washed with brine (20 mL), dried over Na₂SO₄ and concentrated under reduced pressure. The residue was purified by flash column chromatography on silica gel (100-200 mesh silica gel, 0-30% EtOAc in PE) to give tert-butyl(S) (5-(2-((6-bromo-2,3-dihydrobenzofuran-3-yl)(isopropyl)amino)-2-oxoacetamido)-3-methylpyridin-2-yl)(tert-butoxycarbonyl)carbamate (20 mg, 69% yield) as a yellow solid.

LC-MS (ESI): mass calcd, for C₂₉H₃₇BrN₄O₇, 632.18; m/z found, 635.5 [M−H]⁺.

Step F: methyl(S)-5-(3-(2-((6-(bis(tert-butoxycarbonyl)amino)-5-methylpyridin-3-yl)amino)-N-isopropyl-2-oxoacetamido)-2,3-dihydrobenzofuran-6-yl)picolinate

To a solution of tert-butyl(S)-(5-(2-((6-bromo-2,3-dihydrobenzofuran-3-yl)(isopropyl)amino)-2-oxoacetamido)-3-methylpyridin-2-yl)(tert-butoxycarbonyl)carbamate (20 mg, 31.6 μmol, 1.0 eq.), methyl 5-(4,4,5,5-tetramethyl-1,3,2-dioxaborolan-2-yl)picolinate (11 mg, 41.0 μmol, 1.3 eq.) and K₂CO₃ (13 mg, 94.7 μmol, 3.0 eq.) in 1,4-dioxane (3.0 mL) and water (0.3 mL) was added Pd(dppf)Cl₂ (3 mg, 3.16 μmol, 0.1 eq.). The mixture was stirred at 90° C. for 3 hours. The resulting solution was diluted with 10 mL of water and extracted with EtOAc (10 mL×3). The combined organic layers were washed with brine (20 mL), dried over Na₂SO₄ and concentrated under reduced pressure. The residue was purified by flash column chromatography on silica gel (100-200 mesh silica gel, 0-60% EtOAc in PE) to give methyl(S)-5-(3-(2-((6-(bis(tert-butoxycarbonyl)amino)-5-methylpyridin-3-yl)amino)-N-isopropyl-2-oxoacetamido)-2,3-dihydrobenzofuran-6-yl)picolinate (15 mg, 69% yield) as a yellow solid.

LC-MS (ESI): mass calcd, for C₃₆H₄₃N₅O₉, 689.31; m/z found, 689.8 [M−H]⁺.

Step G: (S)-5-(3-(2-((6-(bis(tert-butoxycarbonyl)amino)-5-methylpyridin-3-yl)amino)-N-isopropyl-2-oxoacetamido)-2,3-dihydrobenzofuran-6-yl)picolinic acid

To a solution of methyl(S)-5-(3-(2-((6-(bis(tert-butoxycarbonyl)amino)-5-methylpyridin-3-yl)amino)-N-isopropyl-2-oxoacetamido)-2,3-dihydrobenzofuran-6-yl)picolinate (15 mg, 21.7 μmol, 1.0 eq.) in THF (1.0 mL) and Water (1.0 mL) was added Lithium hydroxide monohydrate (2 mg, 43.5 μmol, 2.0 eq.) and stirred at room temperature for 1 hour. The resulting solution was concentrated under reduced pressure to give(S)-5-(3-(2-((6-(bis(tert-butoxycarbonyl)amino)-5-methylpyridin-3-yl)amino)-N-isopropyl-2-oxoacetamido)-2,3-dihydrobenzofuran-6-yl)picolinic acid (14 mg, 94% yield) as a yellow solid.

LC-MS (ESI): mass calcd, for C₃₅H₄₁N₅O₉, 675.29; m/z found, 676.2 [M−H]⁺.

### Steps H-I

Example A93 was synthesized following the same procedures as described in Example A13 (5.5 mg, 23% yield over 2 steps) as a white solid. LC-MS (ESI): mass calcd. for C₅₇H₆₉FN₁₀O₁₁S, 1120.49; m/z found, 1121.3 [M+H]⁺. ¹H NMR (400 MHz, DMSO-d₆) δ 10.96 (s, 1H), 8.97 (s, 1H), 8.91-8.83 (m, 1H), 8.72-8.68 (m, 1H), 8.49 (s, 1H), 8.28 (s, 1H), 8.23 (d, J=8.1 Hz, 1H), 8.15-7.96 (m, 2H), 7.86-7.67 (m, 3H), 7.41 (d, J=7.4 Hz, 1H), 7.32-7.26 (m, 2H), 7.26-7.21 (m, 2H), 7.03 (s, 1H), 6.96 (d, J=7.6 Hz, 1H), 5.87-5.31 (m, 2H), 4.81-4.76 (m, 1H), 4.72-4.59 (m, 2H), 4.56-4.50 (m, 2H), 4.37-4.34 (m, 1H), 4.31-4.23 (m, 2H), 4.20-4.16 (m, 3H), 3.85-3.80 (m, 3H), 3.68-3.64 (m, 4H), 3.62-3.60 (m, 4H), 2.45 (s, 3H), 2.19 (s, 3H), 2.10-2.07 (m, 1H), 1.93-1.88 (m, 1H), 1.45-1.42 (m, 2H), 1.40-1.38 (m, 1H), 1.35-1.33 (m, 1H), 1.27-1.21 (m, 6H), 0.96 (s, 9H).

### Example A94. 2-amino-N-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N-isobutyl-3-methylquinoline-6-carboxamide

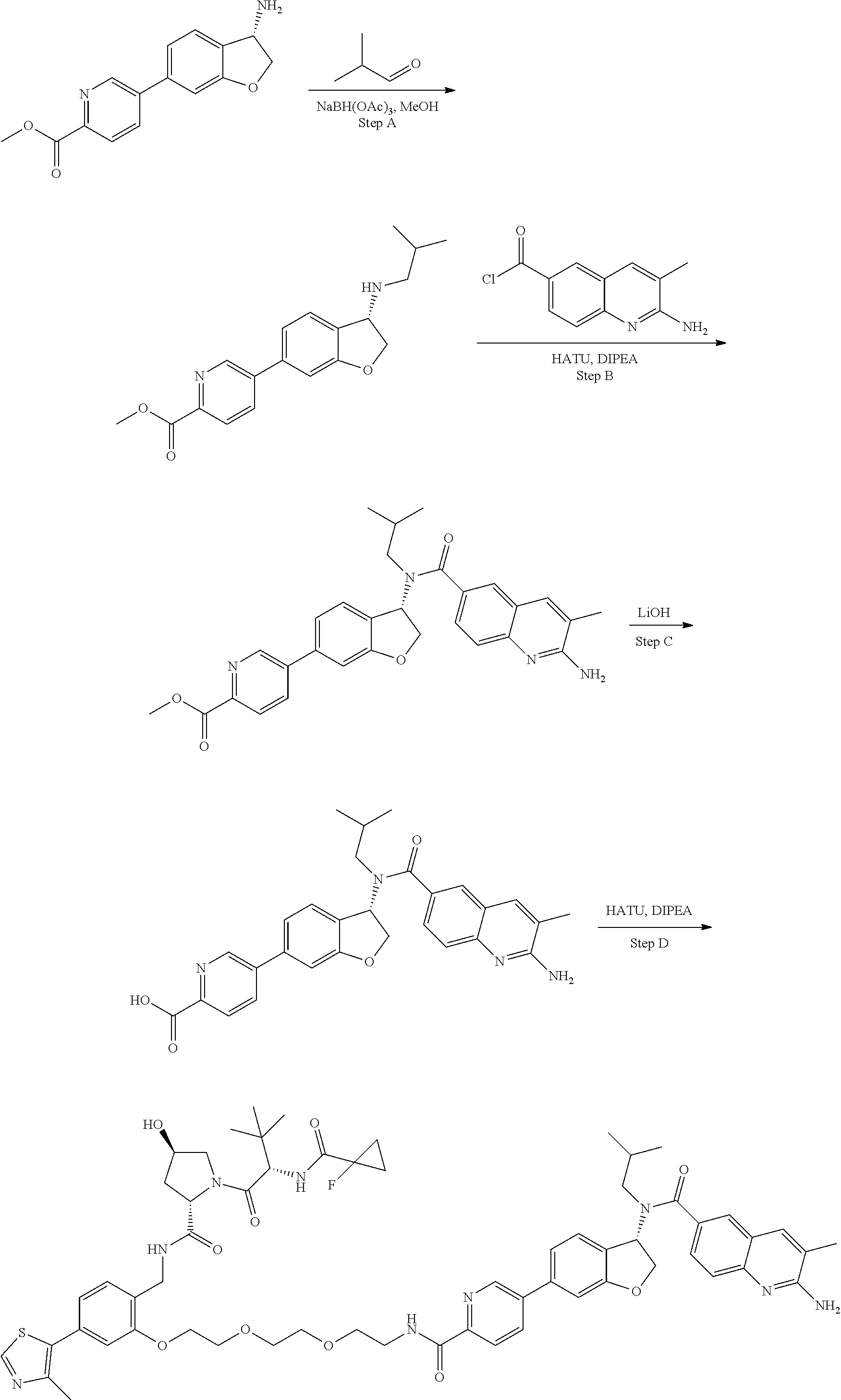

Step A: methyl(S)-5-(3-(isobutylamino)-2,3-dihydrobenzofuran-6-yl)picolinate

To a solution of methyl(S)-5-(3-amino-2,3-dihydrobenzofuran-6-yl)picolinate (70 mg, 259 μmol, 1.0 eq.) in MeOH (10 mL) was added isobutyraldehyde (28 mg, 388 μmol, 1.5 eq.) and NaBH (OAc) 3 (165 mg, 777 μmol, 3.0 eq.). The mixture was stirred at room temperature for 16 hours. The resulting solution was concentrated under reduced pressure. The residue was purified by flash column chromatography on silica gel (100-200 mesh silica gel, 0-10% MeOH in DCM) to give methyl(S)-5-(3-(isobutylamino)-2,3-dihydrobenzofuran-6-yl)picolinate (30 mg, 36% yield) as a white solid.

LC-MS (ESI): mass calcd, for C₁₉H₂₂N₂O₃, 326.16; m/z found, 327.2 [M+H]⁺.

Step B: methyl(S)-5-(3-(2-amino-N-isobutyl-3-methylquinoline-6-carboxamido)-2,3-dihydrobenzofuran-6-yl)picolinate

To a stirred mixture of methyl(S)-5-(3-(isobutylamino)-2,3-dihydrobenzofuran-6-yl)picolinate (30 mg, 92.1 μmol, 1.0 eq.) in DCM (1 mL) and DMF (1.00 mL) were added DIEA (59.4 mg, 41.5 μL, 460 μmol, 5.0 eq.) and 2-amino-3-methylquinoline-6-carbonyl chloride (60.7 mg, 276 μmol, 3.0 eq.) slowly at 0° C. The resulting mixture was stirred at room temperature for 15 hours. The reaction mixture was poured into water (20 mL) and then extracted with DCM (20 mL×2). The combined organic layers were washed with brine, dried over anhydrous Na₂SO₄ and concentrated. The residue was purified by column chromatography (silica gel, 0% EtOAc in PE~10% methanol in DCM) to afford methyl(S)-5-(3-(2-amino-N-isobutyl-3-methylquinoline-6-carboxamido)-2,3-dihydrobenzofuran-6-yl)picolinate (30 mg, 63% yield) as a white solid.

LC-MS (ESI): mass calcd, for C₃₀H₃₀N₄O₄, 510.23; m/z found, 511.1 [M+H]⁺.

Step C: (S)-5-(3-(2-amino-N-isobutyl-3-methylquinoline-6-carboxamido)-2,3-dihydrobenzofuran-6-yl)picolinic acid

To a stirred mixture of methyl(S)-5-(3-(2-amino-N-isobutyl-3-methylquinoline-6-carboxamido)-2,3-dihydrobenzofuran-6-yl)picolinate (30 mg, 58.8 μmol, 1.0 eq.) in THF (1.5 mL) and H₂O (0.5 mL) were added LiOH (5 mg, 117 μmol, 2.0 eq.). The resulting mixture was stirred at room temperature for 1 hour. The mixture was concentrated to give(S)-5-(3-(2-amino-N-isobutyl-3-methylquinoline-6-carboxamido)-2,3-dihydrobenzofuran-6-yl)picolinic acid (29 mg, 99% yield) as a white solid,

LC-MS (ESI): mass calcd, for C₂₉H₂₈N₄O₄, 496.21; m/z found, 497.2 [M+H]⁺.

Step D: 2-amino-N-(S)-6-(6-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N-isobutyl-3-methylquinoline-6-carboxamide

To a stirred mixture of (2S,4R)-N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20 mg, 30.1 μmol, 1.0 eq.) in DMF (1 mL) were added DIEA (12 mg, 8.4 μL, 90.4 μmol, 3.0 eq.), (S)-5-(3-(2-amino-N-isobutyl-3-methylquinoline-6-carboxamido)-2,3-dihydrobenzofuran-6-yl)picolinic acid (14.8 mg, 30.1 μmol, 1.0 eq.) and HATU (14.4 mg, 45.2 μmol, 1.5 eq.). The resulting mixture was stirred at room temperature for 15 hours. The reaction mixture was purified by prep-HPLC (C18, 20~95%, MeCN in H₂O with 0.1% HCOOH) to afford 2-amino-N-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzo furan-3-yl)-N-isobutyl-3-methylquinoline-6-carboxamide (1.4 mg, 4% yield) as a white solid.

LC-MS (ESI): mass calcd, for C₆₁H₂₂FN₉O₁₀S, 1141.51; m/z found, 1142.5 [M+H]⁺. ¹H NMR (400 MHz, DMSO-d₆) δ 8.95 (s, 1H), 8.90 (s, 1H), 8.69 (t, J=5.5 Hz, 1H), 8.48 (t, J=6.0 Hz, 1H), 8.36-8.21 (m, 2H), 8.08 (d, J=8.3 Hz, 1H), 7.97 (s, 1H), 7.83-7.70 (m, 2H), 7.57 (d, J=7.5 Hz, 1H), 7.39 (d, J=7.9 Hz, 1H), 7.34 (d, J=8.4 Hz, 1H), 7.30-7.26 (m, 2H), 7.01 (s, 1H), 6.95 (d, J=7.2 Hz, 1H), 5.34-5.14 (m, 1H), 4.71-4.65 (m, 1H), 4.59 (d, J=9.1 Hz, 1H), 4.51 (t, J=8.1 Hz, 1H), 4.34 (s, 2H), 4.30-4.22 (m, 1H), 4.21-4.11 (m, 3H), 3.84-3.76 (m, 2H), 3.67-3.60 (m, 5H), 3.61-3.56 (m, 4H), 3.54-3.41 (m, 3H), 2.93-2.83 (m, 3H), 2.46 (s, 3H), 2.43 (s, 3H), 2.03-1.90 (m, 3H), 1.39-1.33 (m, 3H), 1.29-1.20 (m, 10H), 0.95 (s, 9H).

### Example A95. 2-amino-N-cyclopentyl-N-((S)-6-(6-((2-(2-(2-(2-(((28,4R)-1-((S)-2-(1-fluorocyclopropane-1-carboxamido)-3,3-dimethylbutanoyl)-4-hydroxypyrr-olidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-3-methylquinoline-6-carboxamide

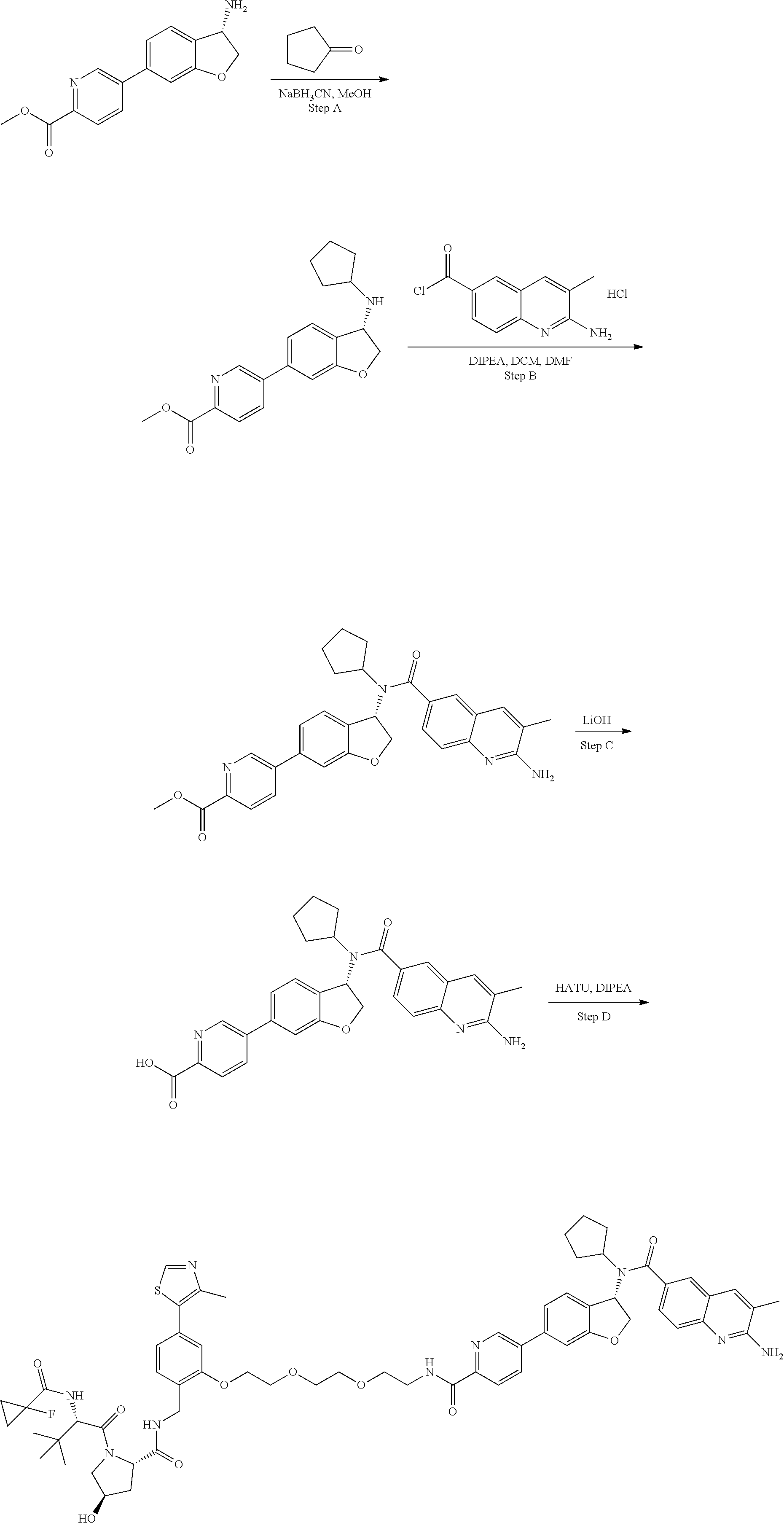

Step A: methyl(S)-5-(3-(cyclopentylamino)-2,3-dihydrobenzofuran-6-yl)picolinate

To a solution of methyl(S)-5-(3-amino-2,3-dihydrobenzofuran-6-yl)picolinate (70 mg, 259 μmol, 1.0 eq.) in MeOH (10 mL) was added cyclopentanone (33 mg, 388 μmol, 1.5 eq.) and NaBH (OAc) 3 (165 mg, 777 μmol, 3.0 eq.). The mixture was stirred at room temperature for 16 hours. The resulting solution was concentrated under reduced pressure. The residue was purified by flash column chromatography on silica gel (100-200 mesh silica gel, 0-10% MeOH in DCM) to give methyl(S)-5-(3-(cyclopentylamino)-2,3-dihydrobenzofuran-6-yl)picolinate (40 mg, 46% yield) as a white solid. LC-MS (ESI): mass calcd, for C₂₀H₂₂N₂O₃, 338.16; m/z found, 339.0 [M+H]⁺.

Step B: methyl(S)-5-(3-(2-amino-N-cyclopentyl-3-methylquinoline-6-carboxamido)-2,3-dihydrobenzofuran-6-yl)picolinate

To a stirred mixture of methyl(S)-5-(3-(cyclopentylamino)-2,3-dihydrobenzofuran-6-yl)picolinate (30 mg, 88.6 μmol, 1.0 eq.) in DCM (1 mL) and DMF (1 mL) were added DIEA (57 mg, 443 μmol, 5.0 eq.) and 2-amino-3-methylquinoline-6-carbonyl chloride (59 mg, 266 μmol, 3.0 eq.) slowly at 0° C. The resulting mixture was stirred at room temperature for 15 hours. The reaction mixture was poured into water (20 mL) and then extracted with DCM (20 mL×2). The combined organic layers were washed with brine, dried over anhydrous Na₂SO₄ and concentrated. The residue was purified by column chromatography (silica gel, 0% EtOAc in PE~10% methanol in DCM) to afford methyl(S)-5-(3-(2-amino-N-cyclopentyl-3-methylquinoline-6-carboxamido)-2,3-dihydrobenzofuran-6-yl)picolinate (30 mg, 65% yield) as a white solid.

LC-MS (ESI): mass calcd, for C₃₁H₃₀N₄O₄, 522.23; m/z found, 523.2 [M+H]⁺.

Step C: (S)-5-(3-(2-amino-N-cyclopentyl-3-methylquinoline-6-carboxamido)-2,3-dihydrobenzofuran-6-yl)picolinic acid

To a stirred mixture of methyl(S)-5-(3-(2-amino-N-cyclopentyl-3-methylquinoline-6-carboxamido)-2,3-dihydrobenzofuran-6-yl)picolinate (30 mg, 57.4 μmol, 1.0 eq.) in THF (1.5 mL) and H₂O (0.5 mL) were added Lithium hydroxide monohydrate (5 mg, 115 μmol, 2.0 eq.). The resulting mixture was stirred at room temperature for 1 hour. The mixture was concentrated to give(S)-5-(3-(2-amino-N-cyclopentyl-3-methylquinoline-6-carboxamido)-2,3-dihydrobenzofuran-6-yl)picolinic acid (29 mg, 99% yield) as a white solid.

LC-MS (ESI): mass calcd, for C₃₀H₂₈N₄O₄, 508.21; m/z found, 509.2 [M+H]⁺.

Step D: 2-amino-N-cyclopentyl-N-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-3-methylquinoline-6-carboxamide

To a stirred mixture of (2S,4R)-N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20 mg, 30.1 μmol, 1.0 eq.) in DMF (1 mL) were added DIEA (12 mg, 90.4 μmol, 3.0 eq.), (S)-5-(3-(2-amino-N-cyclopentyl-3-methylquinoline-6-carboxamido)-2,3-dihydrobenzofuran-6-yl)picolinic acid (15.3 mg, 30.1 μmol, 1.0 eq.) and HATU (14.4 mg, 45.2 μmol, 1.5 eq.). The resulting mixture was stirred at room temperature for 15 hours. The reaction mixture was purified by prep-HPLC (C18, 20~95%, MeCN in H₂O with 0.1% HCOOH) to afford 2-amino-N-cyclopentyl-N-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-3-methylquinoline-6-carboxamide (2.10 mg, 6% yield) as a white solid.

LC-MS (ESI): mass calcd, for C62H72FN₉O₁₀S, 1153.51; m/z found, 1154.5 [M+H]⁺. ¹H NMR (400 MHz, DMSO-d₆) δ 8.95 (s, 1H), 8.88 (s, 1H), 8.69 (t, J=5.7 Hz, 1H), 8.55 (s, 1H), 8.50-8.46 (m, 1H), 8.31 (s, 1H), 8.22 (d, J=7.6 Hz, 1H), 8.07 (d, J=8.2 Hz, 1H), 7.89 (s, 1H), 7.77-7.69 (m, 2H), 7.43-7.35 (m, 2H), 7.30-7.25 (m, 2H), 7.22-7.18 (m, 1H), 7.01 (s, 1H), 6.94 (d, J=7.9 Hz, 1H), 5.48-5.27 (m, 1H), 4.79 (s, 1H), 4.65 (s, 1H), 4.59 (d, J=9.4 Hz, 1H), 4.51 (t, J=8.1 Hz, 1H), 4.33 (s, 1H), 4.28 (d, J=5.4 Hz, 1H), 4.22 (d, J=6.2 Hz, 1H), 4.19-4.16 (m, 2H), 3.82-3.78 (m, 2H), 3.66-3.62 (m, 3H), 3.60-3.55 (m, 5H), 3.51-3.47 (m, 3H), 2.43 (s, 3H), 2.31 (s, 3H), 1.94-1.88 (m, 2H), 1.80-1.70 (m, 4H), 1.44-1.36 (m, 4H), 1.35-1.30 (m, 2H), 1.21-1.18 (m, 2H), 0.94 (s, 9H).

### Example A96. 2-amino-N-cyclobutyl-N-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-3-methylquinoline-6-carboxamide

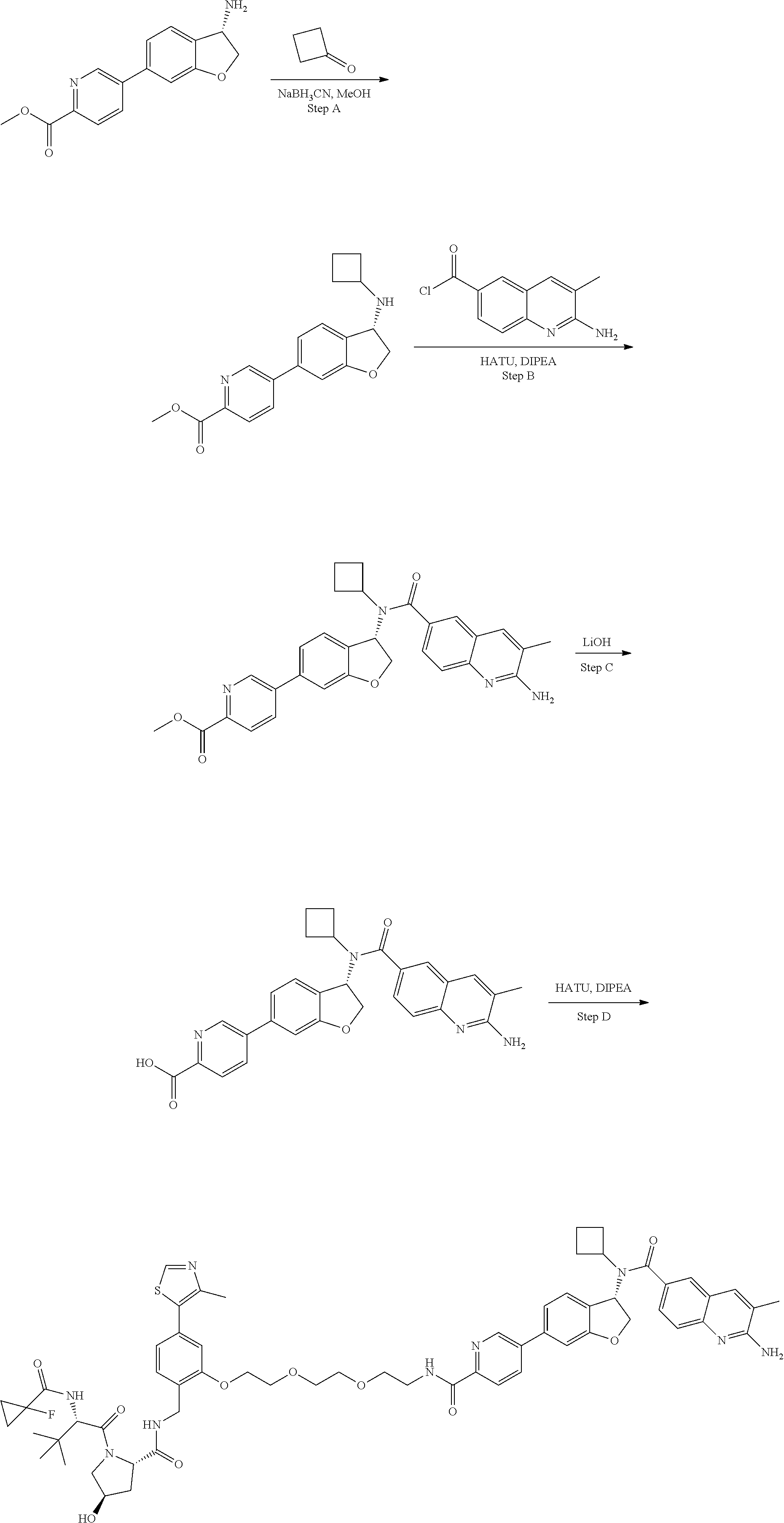

Example A96 was synthesized following the same procedures as described in Example A94 (8.5 mg, 10% yield over 4 steps) as a white solid. LC-MS (ESI): mass calcd. for C₆₁H₇₀FN₉O₁₀S, 1139.50; m/z found, 1140.2 [M+H]⁺. ¹H NMR (400 MHz, DMSO-d₆) δ 8.96 (s, 1H), 8.89 (d, J=1.9 Hz, 1H), 8.74-8.65 (m, 2H), 8.48 (t, J=5.6 Hz, 1H), 8.31 (s, 1H), 8.22 (dd, J=8.1, 2.1 Hz, 1H), 8.07 (d, J=8.2 Hz, 1H), 7.90 (s, 1H), 7.78-7.69 (m, 2H), 7.42-7.35 (m, 2H), 7.31-7.25 (m, 2H), 7.21 (s, 1H), 7.01 (d, J=1.1 Hz, 1H), 6.95 (d, J=7.7 Hz, 1H), 5.80-5.63 (m, 1H), 4.82 (t, J=9.2 Hz, 1H), 4.68-4.63 (m, 1H), 4.59 (d, J=9.1 Hz, 1H), 4.51 (t, J=8.3 Hz, 1H), 4.35-4.33 (m, 1H), 4.28 (d, J=5.7 Hz, 1H), 4.22 (d, J=5.3 Hz, 1H), 4.19-4.15 (m, 3H), 3.81-3.79 (m, 2H), 3.65-3.63 (m, 3H), 3.60-3.56 (m, 5H), 3.52-3.45 (m, 3H), 2.43 (s, 3H), 2.31 (s, 3H), 2.13-2.04 (m, 2H), 1.96-1.87 (m, 2H), 1.65-1.58 (m, 1H), 1.57-1.31 (m, 4H), 1.31-1.13 (m, 3H), 0.94 (s, 9H),

### Example A97. 2-amino-3-cyclobutyl-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-methylquinoline-6-carboxamide

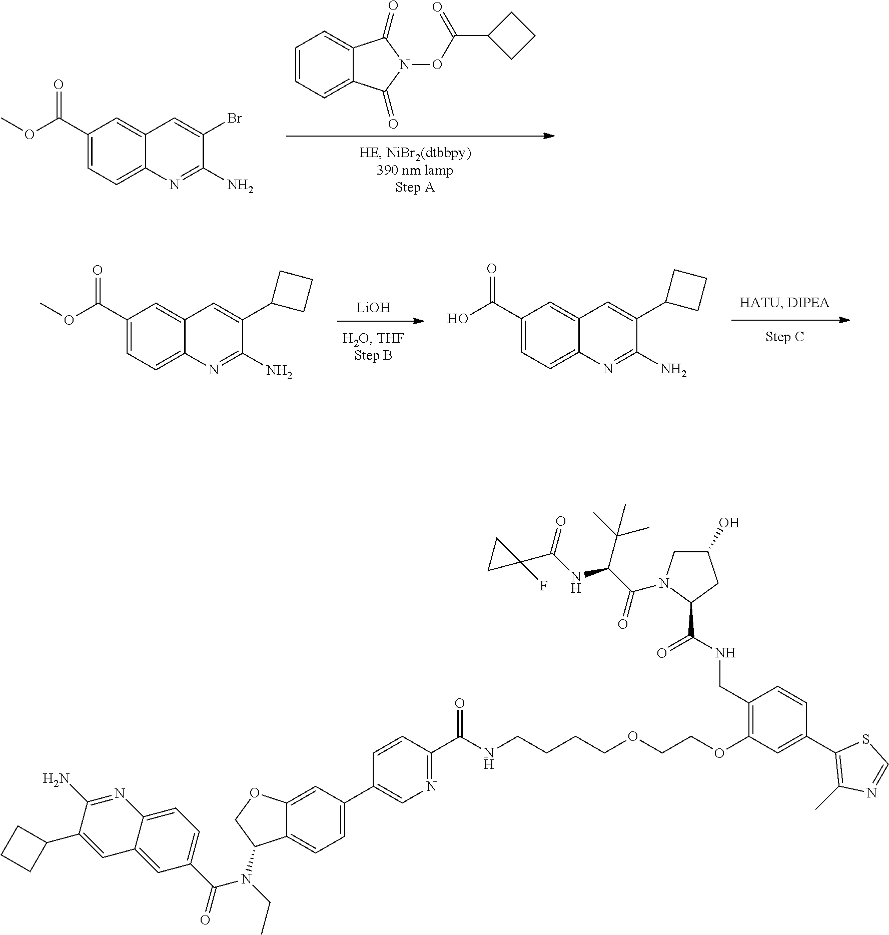 [SMILES: COC(=O)c1ccc2nc(N)c(Br)cc2c1 ; O=C(ON1C(=O)c2ccccc2C1=O)C1CCC1 ; COC(=O)c1ccc2nc(N)c(C3CCC3)cc2c1 ; "LiOH" ; Nc1nc2ccc(C(=O)O)cc2cc1C1CCC1 ; CCN(C(=O)c1ccc2nc(N)c(C3CCC3)cc2c1)[C@@H]1COc2cc(-c3ccc(C(=O)NCCCCOCCOc4cc(-c5scnc5C)ccc4CNC(=O)[C@@H]4C[C@@H](O)CN4C(=O)[C@@H](NC(=O)C4(F)CC4)C(C)(C)C)nc3)ccc21]

Example A97 was synthesized following the same procedures as described in Example

A92 (7.6 mg, 11% yield over 3 steps) as a white solid. LC-MS (ESI): mass calcd. for C₆₁H₇₀FN₉O₉S, 1123.50; m/z found, 1024.6 [M+H]⁺. ¹H NMR (400 MHz, DMSO-d₆) δ 8.97 (s, 1H), 8.92 (s, 1H), 8.81 (s, 1H), 8.54-8.38 (m, 3H), 8.27 (d, J=8.1 Hz, 1H), 8.16 (s, 1H), 8.08 (d, J=8.1 Hz, 1H), 7.88 (d, J=8.6 Hz, 1H), 7.78 (s, 1H), 7.56 (d, J=7.8 Hz, 1H), 7.43-7.34 (m, 3H), 7.29 (d, J=9.2 Hz, 1H), 7.04 (s, 1H), 6.96 (d, J=7.7 Hz, 1H), 4.83 (s, 1H), 4.72-4.68 (m, 1H), 4.59 (d, J=9.1 Hz, 1H), 4.51 (t, J=8.0 Hz, 1H), 4.34 (s, 1H), 4.32-4.22 (m, 2H), 4.21-4.18 (m, 2H), 3.79-3.73 (m, 3H), 3.67-3.63 (m, 4H), 3.36-3.32 (m, 2H), 2.68 (s, 3H), 2.49-2.44 (m, 5H), 2.26-1.96 (m, 5H), 1.96-1.85 (m, 2H), 1.63-1.54 (m, 4H), 1.41-1.32 (m, 2H), 1.24-1.19 (m, 2H), 0.95 (s, 9H).

### Example A98. 2-amino-3-cyano-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-methylquinoline-6-carboxamide

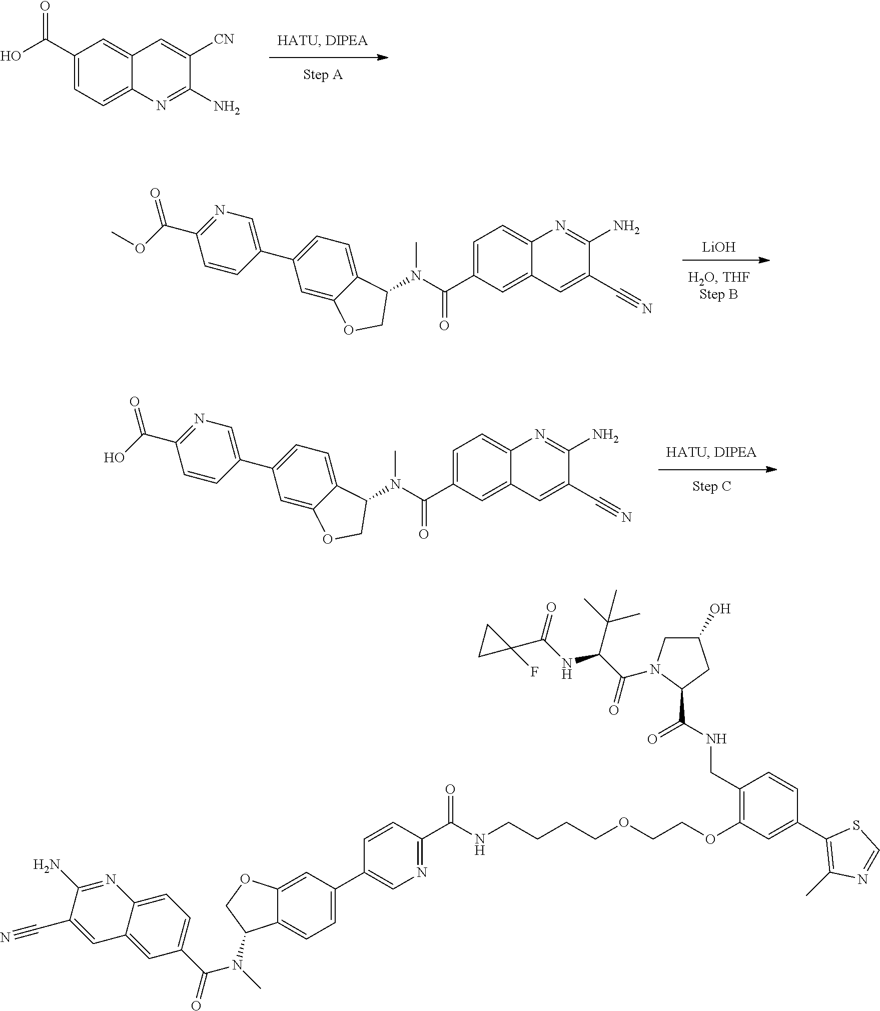 [SMILES: N#Cc1cc2cc(C(=O)O)ccc2nc1N ; COC(=O)c1ccc(-c2ccc3c(c2)OC[C@H]3N(C)C(=O)c2ccc3nc(N)c(C#N)cc3c2)cn1 ; "LiOH" ; CN(C(=O)c1ccc2nc(N)c(C#N)cc2c1)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(C#N)cc4c3)cn2)c1]

Example A98 was synthesized following the same procedures as described in Example A28 (4.6 mg, 8% yield over 3 steps) as a white solid. LC-MS (ESD): mass calcd. for C₅₈H₆₃FN₁₀O₉S, 1094.45; m/z found, 1095.4 [M+H]⁺. ¹H NMR (400 MHz, DMSO-d₆) δ 8.97 (s, 1H), 8.91 (s, 1H), 8.81 (s, 2H), 8.48 (t, J=6.0 Hz, 1H), 8.26 (d, J=8.2 Hz, 1H), 8.08 (d, J=8.1 Hz, 1H), 7.97 (s, 1H), 7.83-7.79 (m, 1H), 7.59 (d, J=9.0 Hz, 1H), 7.55 (d, J=7.7 Hz, 1H), 7.41-7.38 (m, 2H), 7.34 (s, 1H), 7.29 (d, J=9.7 Hz, 1H), 7.04 (s, 1H), 6.96 (d, J=7.6 Hz, 1H), 4.83-4.74 (m, 1H), 4.71-4.68 (m, 1H), 4.59 (d, J=9.2 Hz, 1H), 4.50 (t, J=8.1 Hz, 1H), 4.34 (s, 1H), 4.29-4.22 (m, 2H), 4.21-4.18 (m, 2H), 3.77-3.74 (m, 3H), 3.65-3.62 (m, 4H), 3.34-3.32 (m, 2H), 2.68 (s, 3H), 2.45 (s, 3H), 2.11-2.05 (m, 1H), 1.92 (d, J=8.5 Hz, 1H), 1.62-1.55 (m, 4H), 1.39-1.32 (m, 2H), 1.24-1.20 (m, 2H), 0.95 (s, 9H).

### Example A99. N1-(6-amino-5-methylpyridin-3-yl)-N2-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N2-isopropyloxalamide

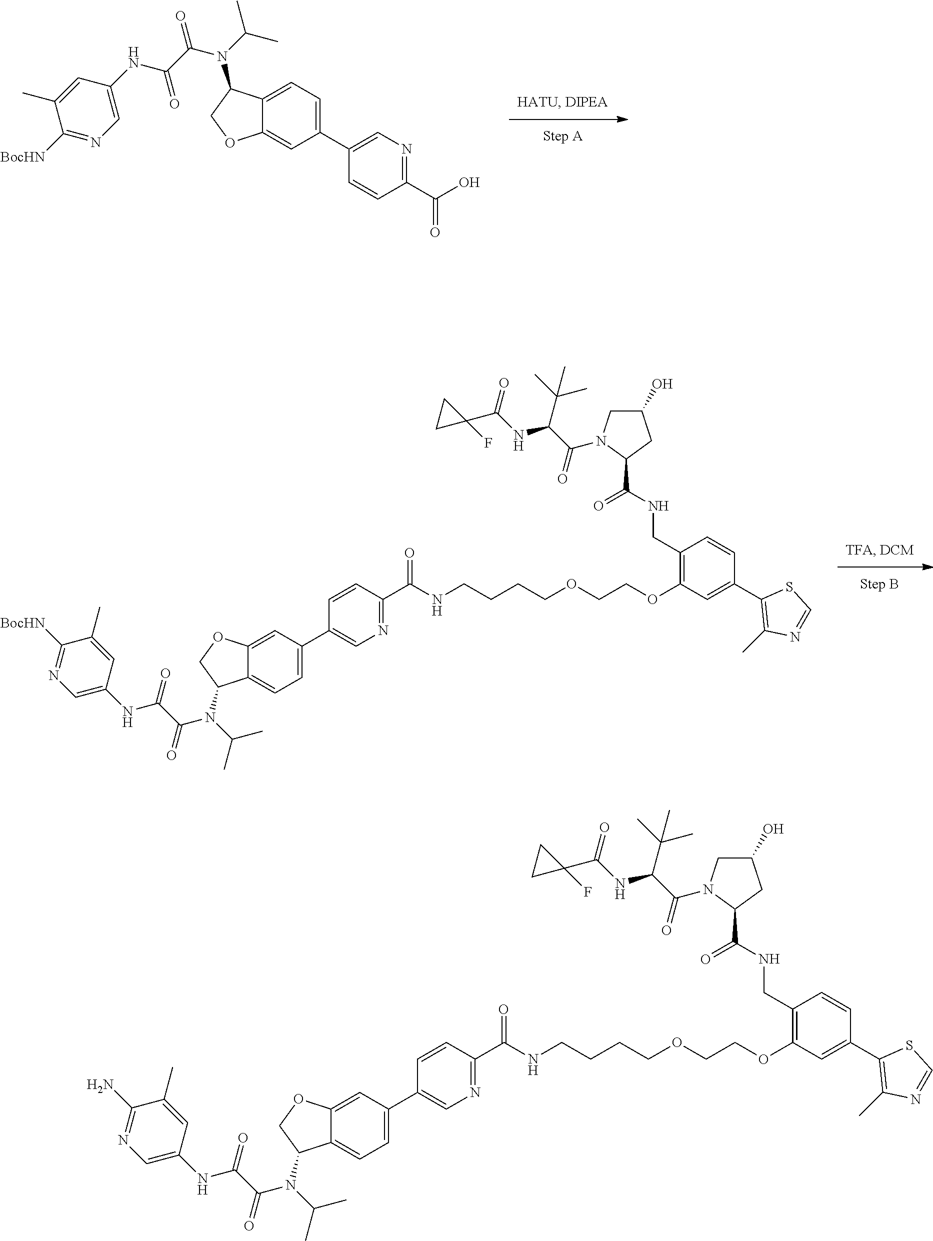 [SMILES: Cc1cc(NC(=O)C(=O)N(C(C)C)[C@@H]2COc3cc(-c4ccc(C(=O)O)nc4)ccc32)cnc1NC(=O)OC(C)(C)C ; Cc1cc(NC(=O)C(=O)N(C(C)C)[C@@H]2COc3cc(-c4ccc(C(=O)NCCCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1NC(=O)OC(C)(C)C ; Cc1cc(NC(=O)C(=O)N(C(C)C)[C@@H]2COc3cc(-c4ccc(C(=O)NCCCCOCCOc5cc(-c6scnc6C)ccc5CNC(=O)[C@@H]5C[C@@H](O)CN5C(=O)[C@@H](NC(=O)C5(F)CC5)C(C)(C)C)nc4)ccc32)cnc1N]

Example A99 was synthesized following the same procedures as described in Example A93 (7.4 mg, 17% yield over 2 steps) as a white solid. LC-MS (ESI): mass calcd. for C₅₇H₆₉FN₁₀O₁₀S, 1104.49; m/z found, 1105.6 [M+H]⁺. ¹H NMR (400 MHz, DMSO-d₆) δ 10.98 (s, 1H), 8.98 (s, 1H), 8.89 (s, 1H), 8.79 (t, J=5.8 Hz, 1H), 8.49 (t, J=5.6 Hz, 1H), 8.29 (s, 1H), 8.23 (d, J=8.3 Hz, 1H), 8.07 (d, J=8.1 Hz, 1H), 7.85 (s, 1H), 7.83-7.68 (m, 2H), 7.41 (d, J=7.8 Hz, 1H), 7.30 (s, 1H), 7.27 (s, 1H), 7.22 (s, 1H), 7.05 (s, 1H), 6.96 (d, J=7.9 Hz, 1H), 5.73-5.44 (m, 1H), 4.80-4.75 (m, 1H), 4.72-4.63 (m, 1H), 4.60 (d, J=9.3 Hz, 1H), 4.57-4.53 (m, 1H), 4.51 (d, J=8.3 Hz, 1H), 4.37-4.33 (m, 2H), 4.29 (d, J=5.8 Hz, 1H), 4.23 (d, J=5.4 Hz, 1H), 4.21-4.18 (m, 3H), 3.76 (s, 4H), 3.34 (d, J=5.8 Hz, 2H), 2.46 (s, 3H), 2.19 (s, 3H), 2.09-2.06 (m, 1H), 1.94-1.89 (m, 1H), 1.59 (s, 4H), 1.43 (d, J=6.3 Hz, 3H), 1.39-1.37 (m, 1H), 1.35-1.33 (m, 1H), 1.26 (d, J=6.5 Hz, 3H), 1.23-1.21 (m, 2H), 0.95 (s, 9H).

### Example A100. N1-(6-amino-5-ethylpyridin-3-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-N2-isopropyloxalamide

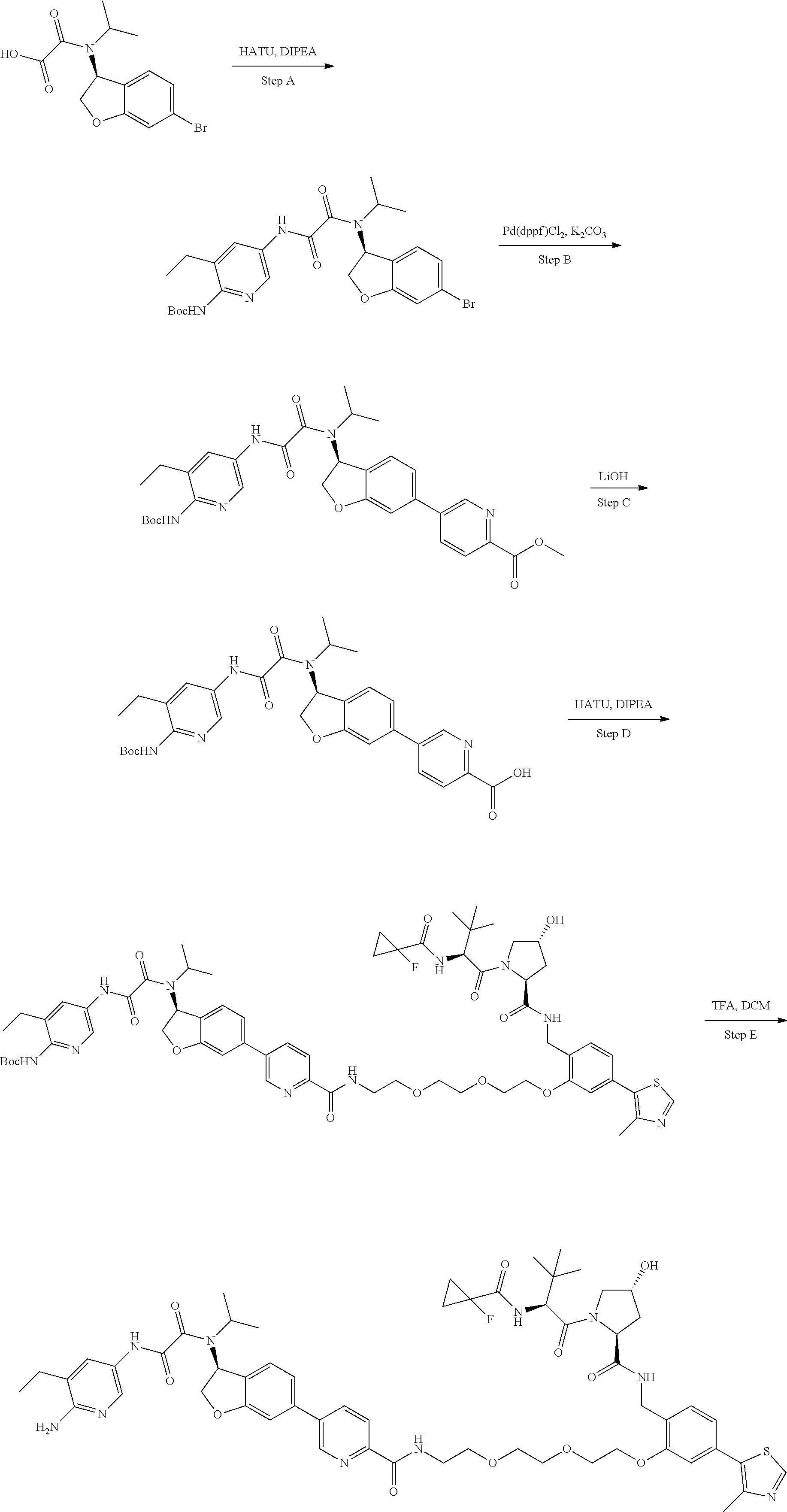

Example A100 was synthesized following the same procedures as described in Example A93 (10.4 mg, 8% yield over 5 steps) as a white solid. LC-MS (ESI): mass calcd. for C₅₈H₇₁FN₁₀O₁₁S, 1134.50; m/z found, 1135.6 [M+H]⁺. ¹H NMR (400 MHz, DMSO-d₆) δ 10.38 (s, 1H), 8.96 (d, J=2.5 Hz, 1H), 8.89 (s, 1H), 8.71 (t, J=5.7 Hz, 1H), 8.49 (t, J=6.4 Hz, 1H), 8.23 (dd, J=8.1, 2.2 Hz, 1H), 8.09-8.04 (m, 2H), 7.48 (s, 1H), 7.40 (d, J=8.0 Hz, 1H), 7.29 (d, J=9.4 Hz, 1H), 7.25 (s, 1H), 7.20 (s, 1H), 7.02 (s, 1H), 6.96 (d, J=8.2 Hz, 1H), 5.73-5.38 (m, 3H), 5.18 (s, 1H), 4.75 (t, J=9.4 Hz, 1H), 4.64-4.55 (m, 2H), 4.52 (t, J=8.0 Hz, 1H), 4.37-4.34 (m, 1H), 4.26 (dd, J=24.2, 5.9 Hz, 2H), 4.20-4.17 (m, 2H), 4.16-4.07 (m, 1H), 3.81 (t, 2H), 3.67-3.63 (m, 3H), 3.62-3.57 (m, 5H), 3.51-3.47 (m, 2H), 2.44 (d, J=3.6 Hz, 3H), 2.40 (d, J=7.4 Hz, 1H), 2.38-2.33 (m, 1H), 2.12-2.06 (m, 1H), 1.96-1.89 (m, 1H), 1.43 (d, J=6.4 Hz, 3H), 1.40-1.38 (m, 1H), 1.36-1.33 (m, 1H), 1.26 (d, J=6.7 Hz, 3H), 1.24-1.20 (m, 2H), 1.12 (t, J=7.4 Hz, 3H), 0.96 (s, 9H).

### Example A101. 2-amino-N-(cyclopropylmethyl)-N-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl) pyridin-3-yl)-2,3-dihydrobenzofuran-3-yl)-3-methylquinoline-6-carboxamide

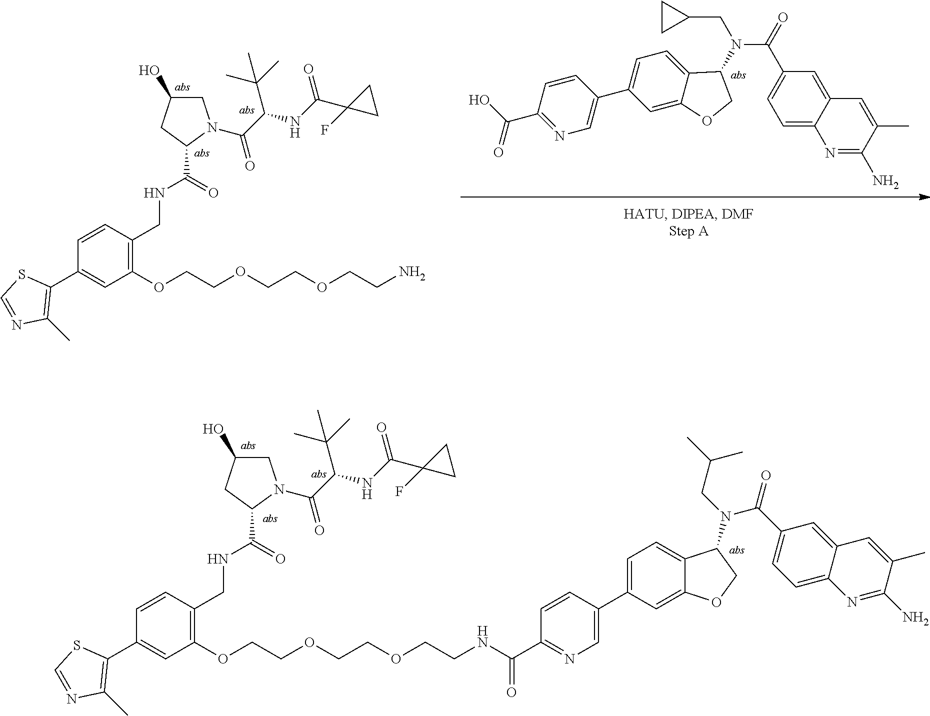 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCN)c1 ; Cc1cc2cc(C(=O)N(CC3CC3)[C@@H]3COc4cc(-c5ccc(C(=O)O)nc5)ccc43)ccc2nc1N ; Cc1cc2cc(C(=O)N(CC(C)C)[C@@H]3COc4cc(-c5ccc(C(=O)NCCOCCOCCOc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N]

To a mixture of(S)-5-(3-(2-amino-N-(cyclopropylmethyl)-3-methylquinoline-6-carboxamido)-2,3-dihydrobenzofuran-6-yl)picolinic acid (9.7 mg, 19.6 μmol, 1.3 eq.), HATU (12 mg, 30.1 μmol, 2.0 eq.) and (2S,4R)-N-(2-(2-(2-(2-aminoethoxy)ethoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (10 mg, 15.1 μmol, 1.0 eq.) in DMF (2 mL) was added DIEA (8 mg, 60.3 μmol, 4.0 eq.). The reaction mixture was purged with nitrogen three times, then stirred at 0° C. for 30 mins under nitrogen. The reaction was monitored by analysis of LC-MS. The reaction mixture was purified by Prep-HPLC to afford 2-amino-N-(cyclopropylmethyl)-N-((S)-6-(6-((2-(2-(2-(2-(((2S,4R)-1-((S)-2-(1-fluorocyclopropane-1-carboxamido)-3,3-dimethylbutanoyl)-4-hydroxy pyrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-3-methylquinoline-6-carboxamide (3.7 mg, 21% yield) as a white solid.

LC-MS (ESI): mass calcd. For C₆₁H₇₀FN₉O₁₀S, 1139.50; m/z found, 1141 [M+H]⁺. ¹HNMR (400 MHz, DMSO-d₆) δ 8.96 (s, 1H), 8.91 (s, 1H), 8.73-8.68 (m, 2H), 8.51-8.45 (m, 1H), 8.32 (s, 1H), 8.27-8.23 (m, 1H), 8.08 (d, J=8.2 Hz, 1H), 8.00 (s, 1H), 7.86-7.80 (m, 1H), 7.79-7.74 (m, 1H), 7.57 (d, J=7.8 Hz, 1H), 7.40 (d, J=7.8 Hz, 1H), 7.35 (d, J=7.9 Hz, 1H), 7.31-7.25 (m, 2H), 7.01 (s, 1H), 6.95 (d, J=7.7 Hz, 1H), 4.86-4.67 (m, 2H), 4.59 (d, J=9.1 Hz, 1H), 4.51 (t, J=8.2 Hz, 1H), 4.36-4.33 (m, 1H), 4.31-4.25 (m, 1H), 4.25-4.19 (m, 1H), 4.19-4.15 (m, 2H), 3.82-3.79 (m, 2H), 3.66-3.63 (m, 3H), 3.62-3.57 (m, 6H), 3.23-3.11 (m, 2H), 3.11-2.96 (m, 2H), 2.43 (s, 3H), 2.32 (s, 3H), 2.11-2.06 (m, 1H), 1.94-1.88 (m, 1H), 1.39-1.32 (m, 2H), 1.30-1.09 (m, 4H), 0.95 (s, 9H), 0.86-0.76 (m, 1H), 0.39-0.26 (m, 2H).

### Example A102. N1-(6-amino-5-ethylpyridin-3-yl)-N2-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N2-isopropyloxalamide

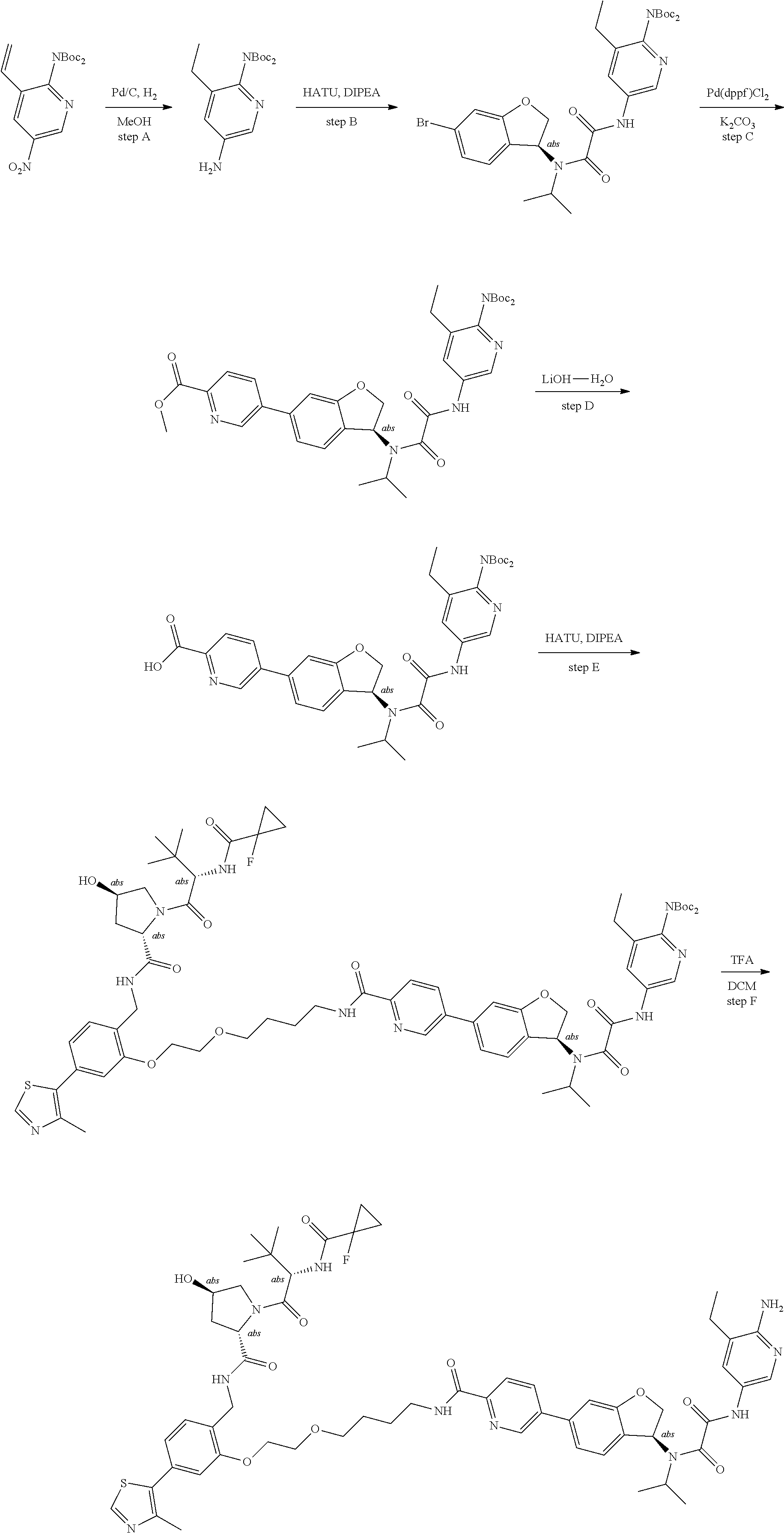

Example A102 was synthesized following the same procedures as described in Example A31 (6.3 mg, 5.5% yield over 6 steps) as a white solid. LC-MS (ESI): mass calcd. for C₅₈H₇₁FN₁₀O₁₀S, 1118.5/z found, 1119.6 [M+H]⁺. ¹HNMR (400 MHz, DMSO-d₆) δ 11.01-10.90 (m, 1H), 8.97 (s, 1H), 8.88 (d, J=1.7 Hz, 1H), 8.78 (t, J=6.0 Hz, 1H), 8.48 (t, 1H), 8.34 (s, 1H), 8.22 (dd, J=8.3, 2.3 Hz, 1H), 8.07 (d, J=8.3 Hz, 1H), 7.81 (s, 1H), 7.78-7.65 (m, 2H), 7.40 (d, J=7.9 Hz, 1H), 7.30-7.25 (m, 3H), 7.22 (s, 1H), 7.04 (s, 1H), 6.96 (d, J=7.9 Hz, 1H), 5.75-5.41 (m, 1H), 4.80-4.75 (m, 1H), 4.59 (d, J=9.1 Hz, 1H), 4.54-4.49 (m, 2H), 4.35-4.33 (m, 1H), 4.29 (d, J=6.2 Hz, 1H), 4.23 (d, J=5.8 Hz, 1H), 4.20-4.18 (m, 3H), 3.77-3.74 (m, 4H), 3.63-3.63 (m, 2H), 3.53-3.52 (m, 2H), 2.45 (s, 3H), 2.09-2.05 (m, 1H), 1.92-1.88 (m, 1H), 1.61-1.56 (m, 4H), 1.43 (d, J=6.6 Hz, 3H), 1.39-1.37 (m, 1H), 1.34-1.32 (m, 1H), 1.26 (d, J=6.5 Hz, 3H), 1.23-1.19 (m, 4H), 1.17 (t, J=7.4 Hz, 3H), 0.95 (s, 9H).

### Example A103. 2-amino-3-bromo-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-methylquinoline-6-carboxamide

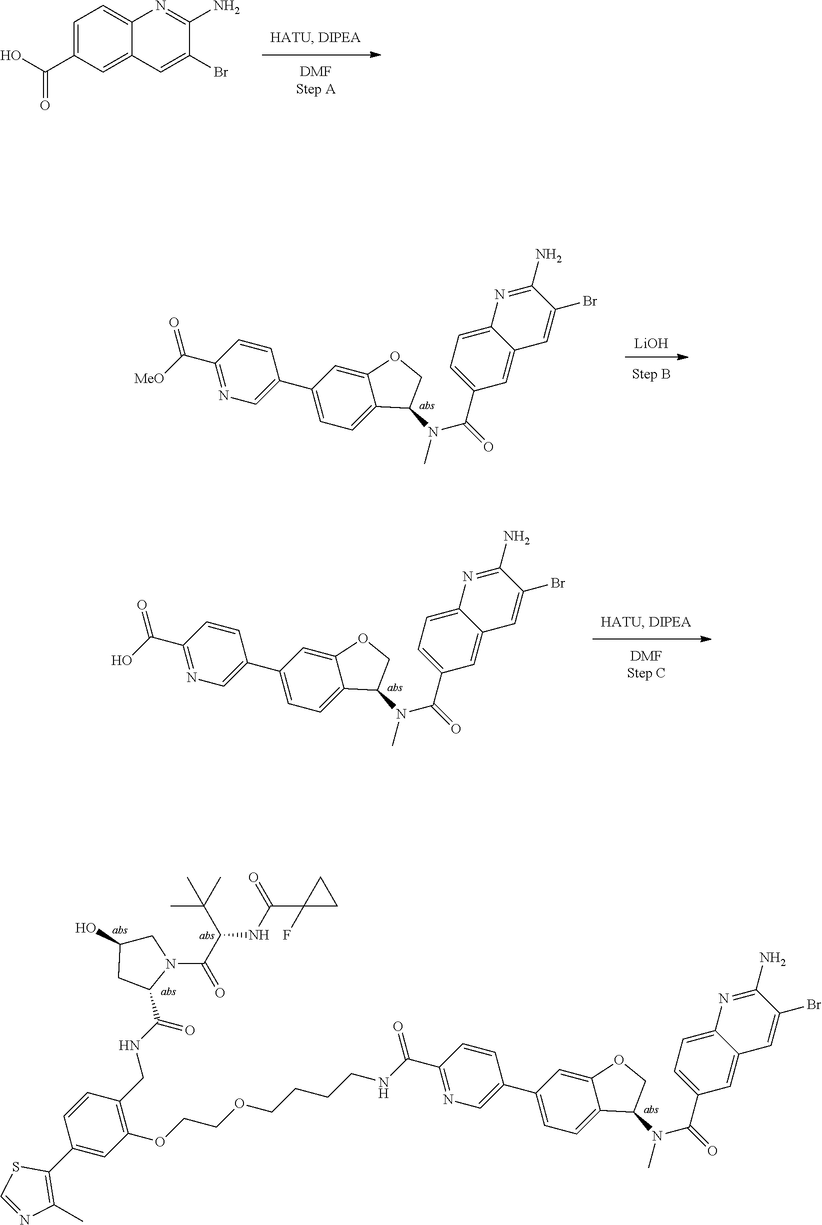 [SMILES: Nc1nc2ccc(C(=O)O)cc2cc1Br ; COC(=O)c1ccc(-c2ccc3c(c2)OC[C@H]3N(C)C(=O)c2ccc3nc(N)c(Br)cc3c2)cn1 ; "LiOH" ; CN(C(=O)c1ccc2nc(N)c(Br)cc2c1)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4nc(N)c(Br)cc4c3)cn2)c1]

Example A103 was synthesized following the same procedures as described in Example A23 (3.8 mg, 5% yield) as a white solid. LC-MS (ESI): mass calcd, for C₅₇H₆₃BrFN₉O₉S, 1147.36; m/z found, 1148.36 [M+H]+. ¹H NMR (400 MHz, DMSO-d₆) δ 8.97 (s, 1H), 8.91 (s, 1H), 8.80 (t, J=6.1 Hz, 1H), 8.59 (s, 1H), 8.48 (t, J=5.9 Hz, 1H), 8.25 (d, J=8.3 Hz, 1H), 8.07 (d, J=8.2 Hz, 1H), 7.91 (s, 1H), 7.72 (d, J=10.0 Hz, 1H), 7.60 (d, J=9.2 Hz, 1H), 7.54 (d, J=7.7 Hz, 1H), 7.41-7.36 (m, 2H), 7.34 (s, 1H), 7.29-7.26 (m, 1H), 7.04 (s, 1H), 6.95 (d, J=8.4 Hz, 1H), 4.87-4.62 (m, 2H), 4.58 (d, J=9.0 Hz, 1H), 4.51 (t, J=8.1 Hz, 1H), 4.36-4.32 (m, 1H), 4.28 (d, J=6.0 Hz, 1H), 4.24-4.17 (m, 3H), 3.77-3.74 (m, 2H), 3.64-3.57 (m, 3H), 3.54-3.51 (m, 4H), 2.67 (s, 3H), 2.44 (s, 3H), 2.07-2.05 (m, 1H), 1.93-1.87 (m, 1H), 1.62-1.54 (m, 4H), 1.38-1.31 (m, 2H), 1.22-1.18 (m, 2H), 0.94 (s, 9H).

### Example A104. 2-amino-3-(tert-butyl)-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-methylquinoline-6-carboxamide

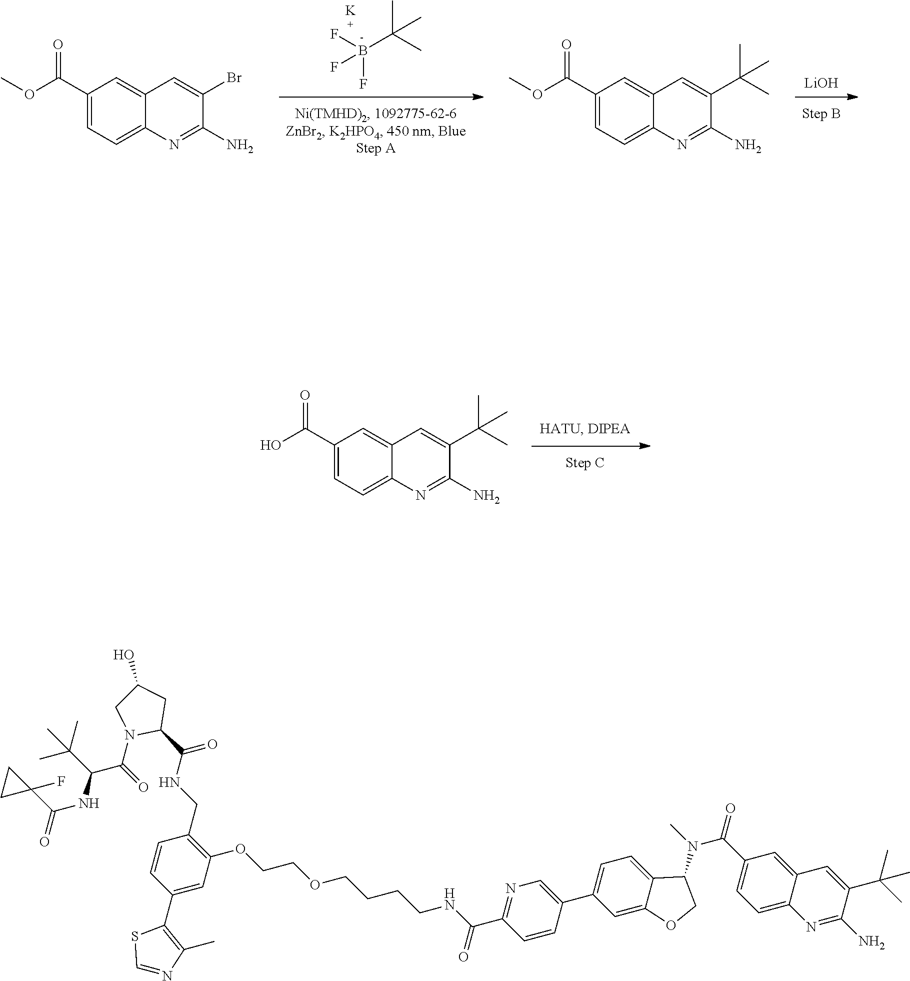

Step A: methyl 2-amino-3-(tert-butyl)quinoline-6-carboxylate

A mixture of methyl 2-amino-3-bromoquinoline-6-carboxylate (50 mg, 178 μmol, 1.0 eq.), [Ir{dFCF₃ppy}₂ (bpy)]PF₆ (2 mg, 1.78 μmol, 0.01 eq.), potassium tert-butyltrifluoroborate (59 mg, 356 μmol, 2.0 eq.), Ni(TMHD) 2 (13 mg, 17.8 μmol, 0.1 eq.), ZnBr₂ (3 mg, 17.8 μmol, 0.1 eq.) and K₂HPO₄ (31 mg, 178 μmol, 1.0 eq.) in DMF (7 mL) was stirred at room temperature for 16 hours under blue LED lights. The resulting solution was diluted with 50 mL of water and extracted with EtO Ac (20 mL×3). The combined organic layers were washed with brine, dried over Na₂SO₄ and concentrated under reduced pressure. The crude product was purified by Prep-HPLC to afford methyl 2-amino-3-(tert-butyl)quinoline-6-carboxylate (5 mg, 11% yield) as a yellow solid. LC-MS (ESI): mass calcd. For C₁₅H₁₈N₂O₂, 258.14; m/z found, 259 [M+H]⁺.

Step B: 2-amino-3-(tert-butyl)quinoline-6-carboxylic acid

A mixture of methyl 2-amino-3-(tert-butyl)quinoline-6-carboxylate (7 mg, 27.1 μmol, 1.0 eq.) and lithium hydroxide (2 mg, 54.2 μmol, 2.0 eq.) in THF (1 mL) and H₂O (1 mL) was stirred at room temperature for 30 mins under nitrogen. The resulting solution was removed under reduced pressure to afford 2-amino-3-(tert-butyl)quinoline-6-carboxylic acid (6.00 mg, 91% yield) as a yellow solid.

LC-MS (ESI): mass calcd. For C₁₄H₁₆N₂O₂, 244.12; m/z found, 245 [M+H]⁺.

Step C: 2-amino-3-(tert-butyl)-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-methylquinoline-6-carboxamide

Example A104 was synthesized following the same procedures as described in Example A23 (1 mg, 3% yield) as a white solid. LC-MS (ESI): mass calcd, for C₆₁H₇₂FN₉O₉S, 1125.52; m/z found, 1127 [M+H]⁺. ¹H NMR (400 MHz, DMSO-d₆) δ 8.97 (s, 1H), 8.91 (s, 1H), 8.81-8.77 (m, 1H), 8.49-8.45 (m, 2H), 8.28-8.23 (m, 2H), 8.21-8.17 (m, 1H), 8.09-8.06 (m, 1H), 7.90-7.87 (m, 1H), 7.83-7.73 (m, 1H), 7.56-7.53 (m, 1H), 7.43-7.33 (m, 4H), 7.28 (d, J=7.3 Hz, 1H), 7.04 (s, 1H), 6.95 (d, J=7.3 Hz, 1H), 4.90-4.62 (m, 2H), 4.60-4.56 (m, 1H), 4.53-4.48 (m, 1H), 4.34-4.32 (m, 1H), 4.31-4.21 (m, 2H), 4.20-4.18 (m, 2H), 3.77-3.74 (m, 2H), 3.64-3.59 (m, 3H), 3.54-3.51 (m, 4H), 2.45 (s, 3H), 2.07 (s, 3H), 2.02-1.99 (m, 1H), 1.92-1.89 (m, 1H), 1.61-1.57 (m, 4H), 1.45 (s, 9H), 1.31-1.27 (m, 2H), 1.22-1.20 (m, 2H), 0.94 (s, 9H).

### Example A105. N1-(6-amino-5-ethylpyridin-3-yl)-N2-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N2-methyloxalamide

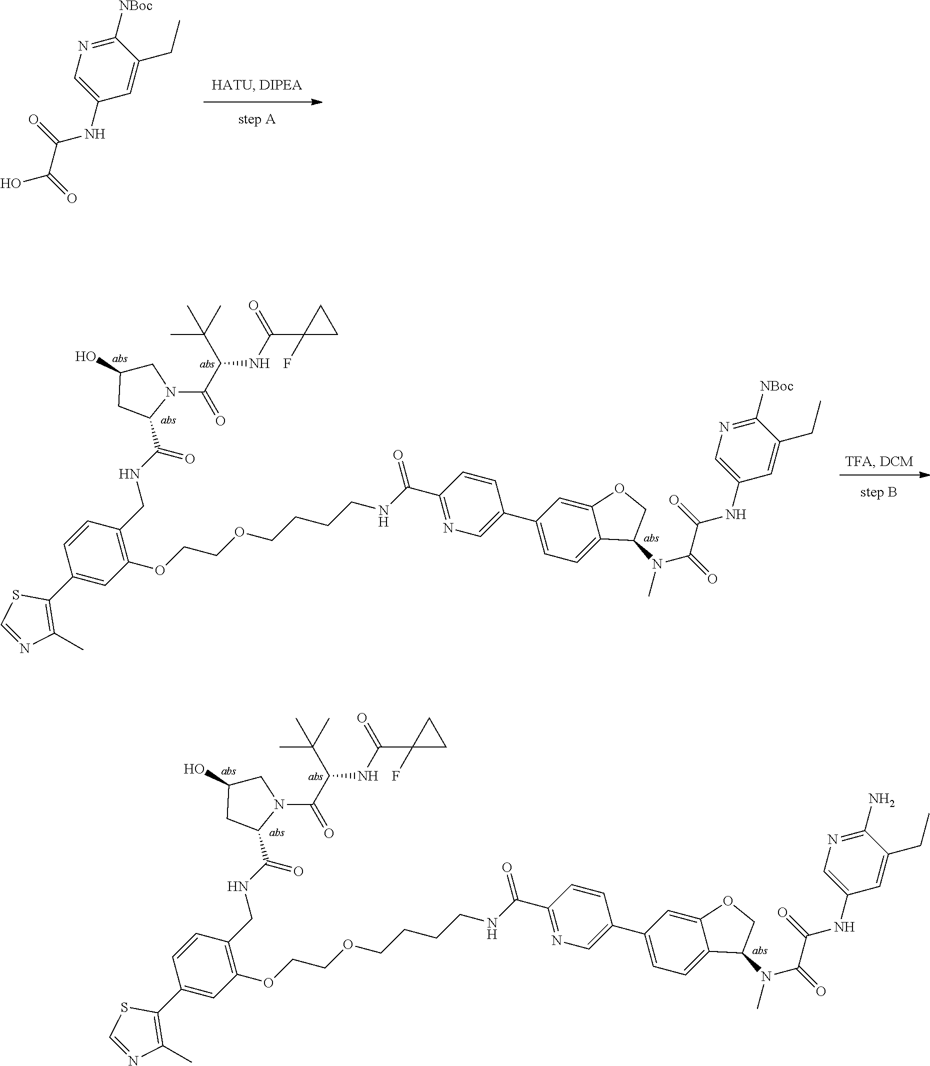

Example A105 was synthesized following the same procedures as described in Example A31 (9.7 mg, 42% yield over 2 steps) as a white solid. LC-MS (ESI): mass calcd. for C56H67FN₁₀O₁₀S, 1090.5/z found, 1092.6 [M+H]⁺. ¹HNMR (400 MHz, DMSO-d₆) δ 11.09 (d, J=37.5 Hz, 1H), 8.97 (s, 1H), 8.91 (s, 1H), 8.81 (t, J=6.0 Hz, 1H), 8.48 (t, J=5.9 Hz, 1H), 8.34 (dd, J=14.8, 2.0 Hz, 1H), 8.28-8.24 (m, 1H), 8.08 (d, J=8.1 Hz, 1H), 7.90-7.83 (m, 1H), 7.68.-7.53 (m, 2H), 7.48-7.34 (m, 4H), 7.28 (dd, J=9.1, 2.7 Hz, 1H), 7.04 (s, 1H), 6.95 (dd, J=7.8, 1.2 Hz, 1H), 6.11 (ddd, J=124.5, 8.6, 3.8 Hz, 1H), 5.37-4.96 (m, 1H), 4.79-4.63 (m, 2H), 4.59 (d, J=9.2 Hz, 1H), 4.51 (t, J=8.2 Hz, 1H), 4.36-4.33 (m, 1H), 4.33-4.21 (m, 2H), 4.21-4.18 (m, 2H), 3.77-3.74 (m, 2H), 3.66-3.57 (m, 3H), 3.54-3.50 (m, 3H), 2.77-2.62 (m, 3H), 2.57-2.54 (m, 2H), 2.45 (s, 3H), 2.11-2.04 (m, 1H), 1.95-1.87 (m, 1H), 1.63-1.55 (m, 4H), 1.40-1.32 (m, 2H), 1.24-1.20 (m, 2H), 1.20-1.16 (m, 3H), 0.94 (s, 9H).

### Example A106. N-((S)-6-(6-((4-(2-(5-ethynyl-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)butyl)carbamoyl)pyridin-3-yl)-2,3-dihydrobenzofuran-3-yl)-N,3-dimethylquinoline-6-carboxamide

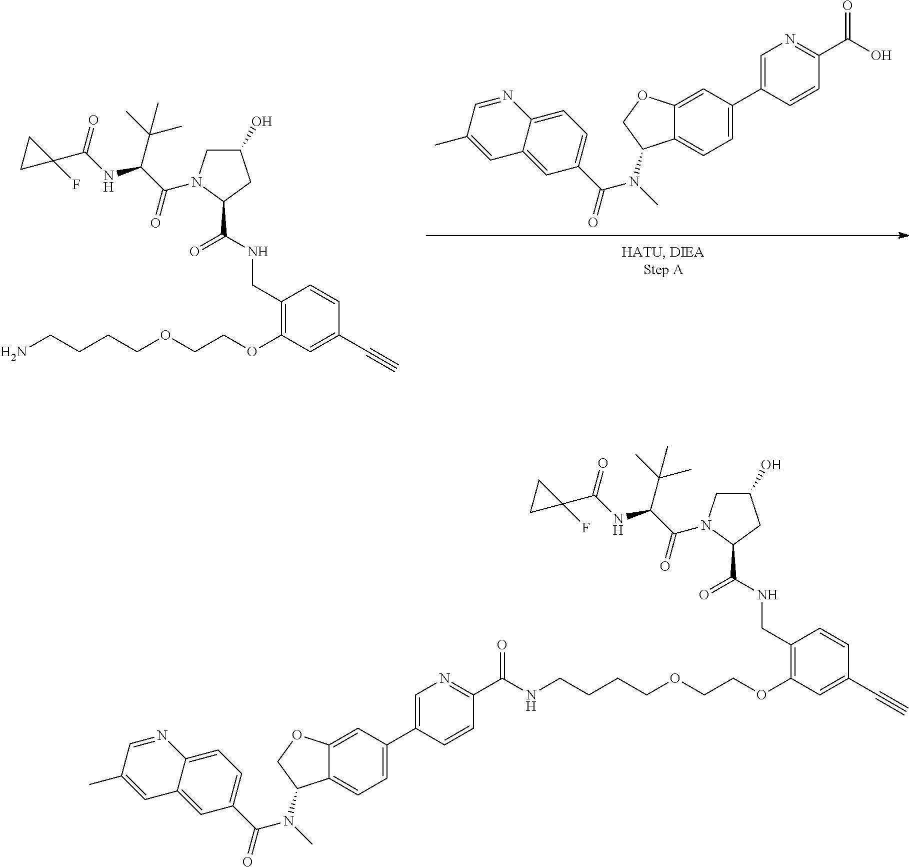 [SMILES: C#Cc1ccc(CNC(=O)[C@@H]2C[C@@H](O)CN2C(=O)[C@@H](NC(=O)C2(F)CC2)C(C)(C)C)c(OCCOCCCCN)c1 ; Cc1cnc2ccc(C(=O)N(C)[C@@H]3COc4cc(-c5ccc(C(=O)O)nc5)ccc43)cc2c1 ; C#Cc1ccc(CNC(=O)[C@@H]2C[C@@H](O)CN2C(=O)[C@@H](NC(=O)C2(F)CC2)C(C)(C)C)c(OCCOCCCCNC(=O)c2ccc(-c3ccc4c(c3)OC[C@H]4N(C)C(=O)c3ccc4ncc(C)cc4c3)cn2)c1]

Example A106 was synthesized following the same procedures as described in Example A39 (3.0 mg, 12% yield over 1 step) as a white solid. LC-MS (ESI): mass calcd, for C₅₆H₆₂FN₇O₉, 995.5/z found, 996.4 [M+H]⁺, ¹HNMR (400 MHz, DMSO-d₆) δ 8.92 (s, 2H), 8.85-8.76 (m, 1H), 8.56-8.42 (m, 1H), 8.41-8.31 (m, 1H), 8.27 (d, J=7.9 Hz, 1H), 8.16-8.05 (m, 3H), 7.85 (d, J=8.5 Hz, 1H), 7.59 (d, J=7.7 Hz, 1H), 7.44-7.23 (m, 4H), 7.05 (s, 1H), 6.96 (d, J=8.0 Hz, 1H), 4.75-4.69 (m, 1H), 4.58 (d, J=9.3 Hz, 1H), 4.51-4.46 (m, 1H), 4.33 (s, 1H), 4.30-4.23 (m, 2H), 4.20-4.17 (m, 1H), 4.16-4.13 (m, 3H), 4.12 (s, 1H), 3.74-3.72 (m, 2H), 3.64-3.58 (m, 2H), 3.53-3.49 (m, 2H), 3.36-3.31 (m, 2H), 2.80-2.59 (m, 3H), 2.54 (s, 3H), 2.09-2.02 (m, 1H), 1.93-1.84 (m, 1H), 1.63-1.53 (m, 4H), 1.41-1.30 (m, 2H), 1.24-1.19 (m, 2H), 0.96-0.91 (m, 9H).

### Example A107. N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,3-dimethylquinoline-6-carboxamide

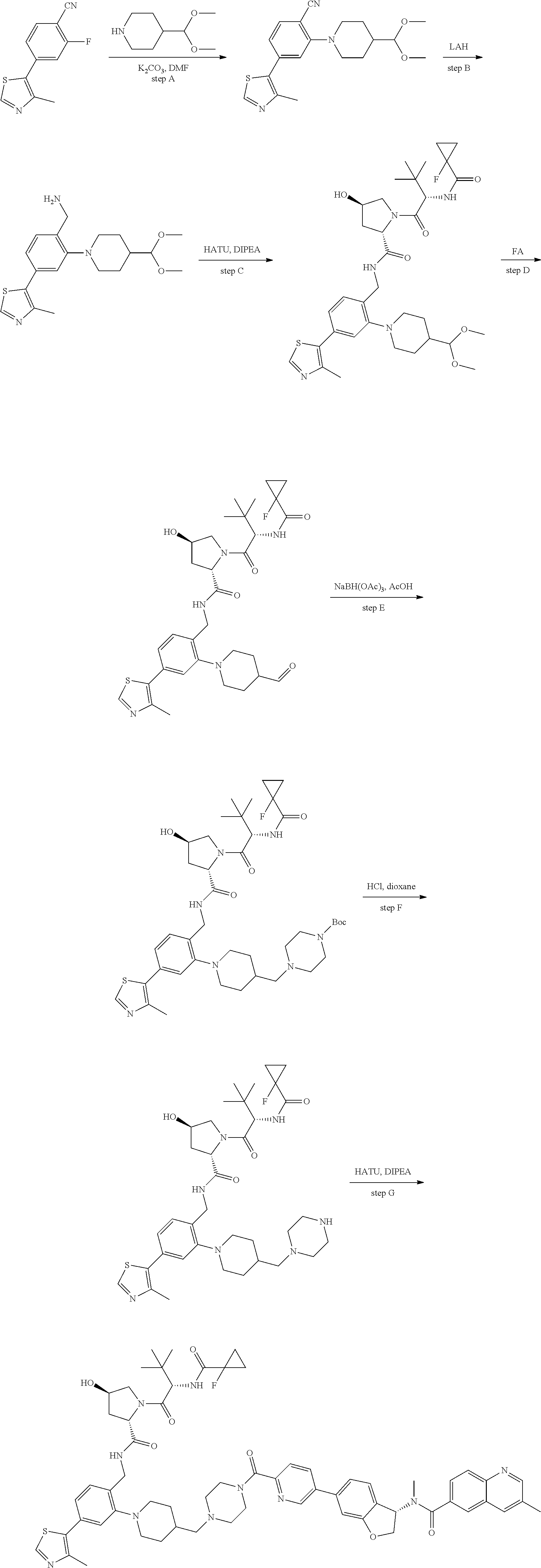

Step A: 2-(4-(dimethoxymethyl)piperidin-1-yl)-4-(4-methylthiazol-5-yl)benzonitrile

To a solution of 2-fluoro-4-(4-methylthiazol-5-yl)benzonitrile (5 g, 22.9 mmol, 1.0 eq.), 4-(dimethoxymethyl)piperidine (7.3 g, 45.8 mmol, 2.0 eq.) in DMF (50.0 mL) was added K₂CO₃ (9.4 g, 68.8 mmol, 3.0 eq.). The mixture was stirred at 80° C. for 6 hours. The resulting solution was diluted with 60 mL of water and extracted with EtOAc (60 mL×3). The combined organic layers were washed with brine (50 mL), dried over Na₂SO₄ and concentrated under reduced pressure. The residue was purified by flash column chromatography on silica gel (100-200 mesh silica gel, 0-20% EtOAc in PE) to give 2-(4-(dimethoxymethyl)piperidin-1-yl)-4-(4-methylthiazol-5-yl)benzonitrile (3.8 g, 47% yield) as a yellow solid. LC-MS (ESI): mass calcd. for C19H23N302S, 357.15; m/z found, 358 [M+H]⁺.

Step B: (2-(4-(dimethoxymethyl)piperidin-1-yl)-4-(4-methylthiazol-5-yl)phenyl)methanamine

To a solution of 2-(4-(dimethoxymethyl)piperidin-1-yl)-4-(4-methylthiazol-5-yl)benzonitrile (500 mg, 1.40 mmol, 1.0 eq.) in THF (15 mL) at 0° C. The mixture was stirred at room temperature for 16 hours under nitrogen. After cooling to room temperature, The resulting solution was quenched with Na₂SO₄·10H₂O and filtrated to afford the crude (2-(4-(dimethoxymethyl)piperidin-1-yl)-4-(4-methylthiazol-5-yl)phenyl)methanamine (360 mg, 28% yield) as a yellow solid. LC-MS (ESI): mass calcd. For C19H27N302S, 361.1; m/z found, 362.3 [M+H]⁺.

Step C: (2S,4R)-N-(2-(4-(dimethoxymethyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

A mixture of (2-(4-(dimethoxymethyl)piperidin-1-yl)-4-(4-methylthiazol-5-yl)phenyl)methanamine (360 mg, 1.0 mmol, 1.0 eq.), (2S,4R)-1-((S)-2-(1-fluorocyclopropane-1-carboxamido)-3,3-dimethylbutanoyl)-4-hydroxypyrrolidine-2-carboxylic acid (329 mg, 1.0 mmol, 1.0 eq.), HATU (756 mg, 1.99 mmol, 2.0 eq.) and DIPEA (0.5 mL, 2.99 mmol, 3.0 eq.) in DMF (10 mL) was stirred at room temperature for 30 min under nitrogen. The resulting solution was diluted with 50 mL of water and extracted with EtOAc (30 mL×3). The combined organic layers were washed with brine, dried over Na₂SO₄ and concentrated under reduced pressure. The crude product was purified by flash column chromatography on silica gel (100-200 mesh silica gel, 0~10% MeOH in DCM) to afford (2S,4R)-N-(2-(4-(dimethoxymethyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (110 mg, 21% yield) as a yellow solid.

LC-MS (ESI): mass calcd. For C19H24N204S, 673.3; m/z found, 674.9 [M+H]⁺.

Step D: (2S,4R)-1-((S)-2-(1-fluorocyclopropane-1-carboxamido)-3,3-dimethylbutanoyl)-N-(2-(4-formylpiperidin-1-yl)-4-(4-methylthiazol-5-yl)benzyl)-4-hydroxypyrrolidine-2-carboxamide

To a solution of (2S,4R)-N-(2-(4-(dimethoxymethyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (110 mg, 163 μmol, 1.0 eq.) in HCOOH (3 mL) was stirred at 55° C. for 1 hour under nitrogen. After cooling to room temperature, The resulting solution was removed under reduced pressure to afford the crude (28,4R)-1-((S)-2-(1-fluorocyclopropane-1-carboxamido)-3,3-dimethylbutanoyl)-N-(2-(4-formylpiperidin-1-yl)-4-(4-methylthiazol-5-yl)benzyl)-4-hydroxypyrrolidine-2-carboxamide (100 mg, 48% yield) as a yellow oil.

LC-MS (ESI): mass calcd. For C₃₂H₄₂FN₅O₅S, 627.3; m/z found, 678.4 [M+H]⁺.

Step E: tert-butyl 4-((1-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carboxylate

To a solution of (2S,4R)-1-((S)-2-(1-fluorocyclopropane-1-carboxamido)-3,3-dimethylbutanoyl)-N-(2-(4-formylpiperidin-1-yl)-4-(4-methylthiazol-5-yl)benzyl)-4-hydroxypyrrolidine-2-carboxamide (100 mg, 159 μmol. 1.0 eq.),tert-butyl piperazine-1-carboxylate (45 mg, 239 μmol, 1.5 eq.) and AcOH (5 mg, 79.6 μmol, 0.5 eq.) in MeOH (3 mL) was stirred at room temperature for 1 hour under nitrogen. Then NaBH₃CN (20 mg, 319 μmol, 2.0 eq.) was added and stirred at room temperature for 1 hour. After cooling to room temperature, the resulting solution was diluted with 20 mL of water and extracted with EtOAc. The combined organic layers were washed with brine, dried over Na₂SO₄ and concentrated under reduced pressure. The crude product was purified by flash column chromatography on silica gel (100-200 mesh silica gel, 0~5% MeOH in DCM) to afford tert-butyl 4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carboxylate (30 mg, 24% yield) as a yellow solid. LC-MS (ESI): mass calcd. For C₄₁H₆₀FN₇O₆S, 797.4; m/z found, 798.6 [M+H]⁺.

Steps F-G: N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,3-dimethylquinoline-6-carboxamide

Example A107 was synthesized following the same procedures as described in Example A9 (7.5 mg, 44% yield over 2 steps) as a white solid. LC-MS (ESI): mass calcd. for C₆₂H₇₁FN₁₀O₇S, 1118.5; m/z found, 1119.6 [M+H]⁺. ¹H NMR (400 MHz, DMSO-d₆) δ 8.98 (s, 1H), 8.92 (s, 1H), 8.85 (d, J=2.0 Hz, 1H), 8.56-8.43 (m, 1H), 8.23 (s, 2H), 8.11-8.04 (m, 2H), 7.80 (d, J=8.3 Hz, 1H), 7.58 (d, 1H), 7.46-7.37 (m, 2H), 7.34 (s, 1H), 7.28 (d, J=6.3 Hz, 1H), 7.12 (s, 2H), 5.17 (d, 1H), 4.71 (dd, 1H), 4.60 (d, J=9.2 Hz, 1H), 4.49 (t, J=8.2 Hz, 1H), 4.38-4.32 (m, 2H), 3.71-3.58 (m, 4H), 3.50-3.41 (m, 4H), 3.27-3.24 (m, 3H), 3.20-3.15 (m, 2H), 3.10-3.04 (m, 2H), 2.74-2.63 (m, 6H), 2.56-2.52 (m, 4H), 2.46 (s, 3H), 2.39-2.36 (m, 1H), 2.34-2.31 (m, 1H), 2.27-2.21 (m, 1H), 2.11-2.04 (m, 1H), 1.91-1.82 (m, 2H), 1.40-1.34 (m, 2H), 1.24-1.19 (m, 2H), 0.97 (s, 9H).

### Example A108. N-((S)-6-(6-(4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-3,6-dihydropyridin-1 (2H)-yl)methyl)piperidine-1-carbonyl)pyridin-3-yl)-2,3-dihydrobenzofuran-3-yl)-N,3-dimethylquinoline-6-carboxamide

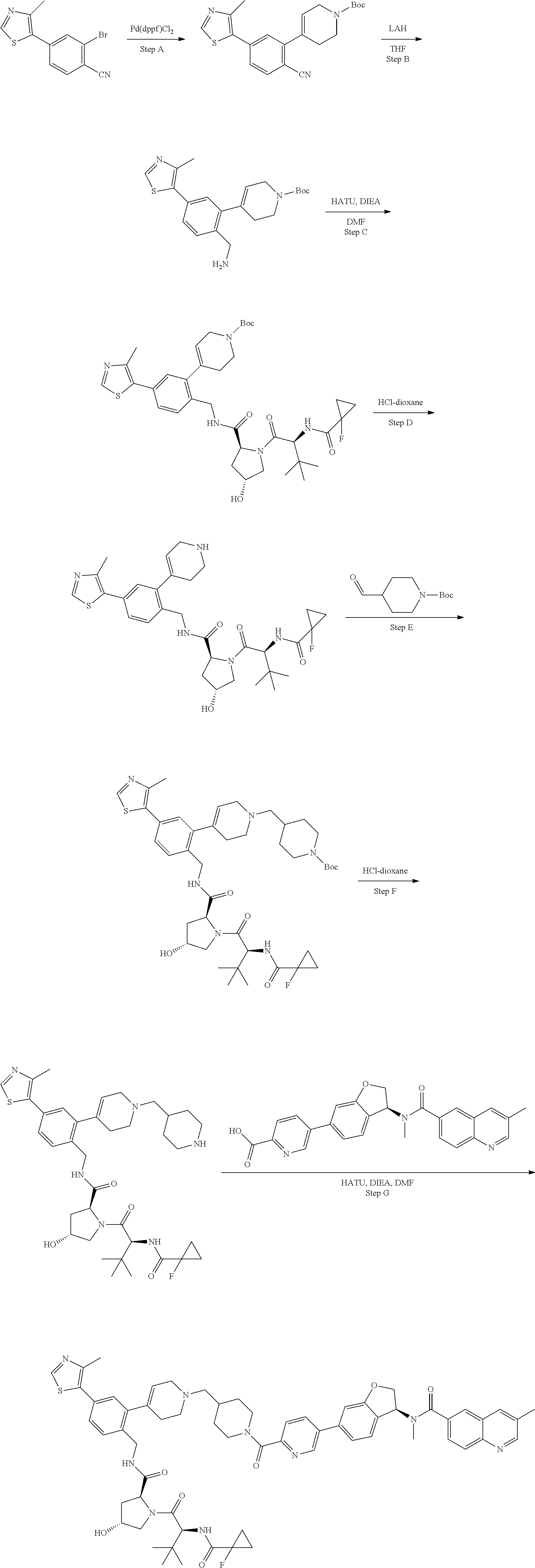

Step A: tert-butyl 4-(2-cyano-5-(4-methylthiazol-5-yl)phenyl)-3,6-dihydropyridine-1 (2H)-carboxylate

A solution of 2-cyano-5-(4-methylthiazol-5-yl) phenyl trifluoromethanesulfonate (1.2 g, 3.4 mmol, 1.0 eq.) and K₂CO₃ (1.4 g, 10 mmol, 3.0 eq.) in 1,4-Dioxane (30 mL) and Water (6 mL) was added Bis-(triphenylphosphino)-palladous chloride (240 mg, 0.34 mmol, 0.1 eq.) and tert-butyl 4-(4,4,5,5-tetramethyl-1,3,2-dioxaborolan-2-yl)-3,6-dihydropyridine-1 (2H)-carboxylate (1.6 g, 5.2 mmol, 1.5 eq.). The reaction was stirred at 80° C. for 3 hrs. The resulting solution was diluted with 100 mL of water and extracted with EtOAc. The combined organic layers were washed with brine, dried over Na₂SO₄ and concentrated under reduced pressure. The residue was purified by flash column chromatography on silica gel (100-200 mesh silica gel, 0-34% EtOAc in PE) to give tert-butyl 4-(2-cyano-5-(4-methylthiazol-5-yl)phenyl)-3,6-dihydropyridine-1 (2H)-carboxylate (1.2 g, 82% yield) as a yellow oil. LC-MS (ESI): mass calcd. For C21H₂₃N302S, 381.15; m/z found, 382 [M+H]⁺.

Steps B-G: N-((S)-6-(6-(4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-3,6-dihydropyridin-1 (2H)-yl)methyl)piperidine-1-carbonyl)pyridin-3-yl)-2,3-dihydrobenzofuran-3-yl)-N,3-dimethylquinoline-6-carboxamide

Example A108 was synthesized following the same procedures as described in Example A13 (3.3 mg, 2% yield over 6 steps) as a white solid. LC-MS (ESI): mass calcd, for C₆₃H₇₀FN₉O₇S, 1115.51; m/z found, 1116.8 [M+H]⁺. ¹H NMR (400 MHz, DMSO-d₆) δ 9.46 (s, 1H), 9.01 (s, 1H), 8.90 (s, 2H), 8.61-8.53 (m, 1H), 8.30 (s, 1H), 8.25-8.21 (m, 1H), 8.13-8.08 (m, 2H), 7.85-7.81 (m, 1H), 7.66-7.63 (m, 1H), 7.60-7.56 (m, 1H), 7.54-7.51 (m, 1H), 7.42-7.38 (m, 2H), 7.34 (s, 1H), 7.27-7.23 (m, 2H), 5.70 (s, 1H), 4.86-4.72 (m, 2H), 4.61-4.58 (m, 1H), 4.56-4.51 (m, 1H), 4.49-4.46 (m, 1H), 4.39-4.29 (m, 3H), 4.08-4.01 (m, 2H), 3.87-3.80 (m, 4H), 3.20-3.15 (m, 3H), 2.92-2.85 (m, 2H), 2.69-2.66 (m, 2H), 2.53 (s, 3H), 2.46 (s, 3H), 2.27-2.17 (m, 2H), 1.95-1.74 (m, 4H), 1.39-1.20 (m, 9H), 0.97 (s, 9H).

### Example A109. 2-amino-N-((S)-6-(6-(((1-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)piperidin-4-yl)methyl)carbamoyl)pyridin-3-yl)-2,3-dihydrobenzofuran-3-yl)-N,3-dimethylquinoline-6-carboxamide

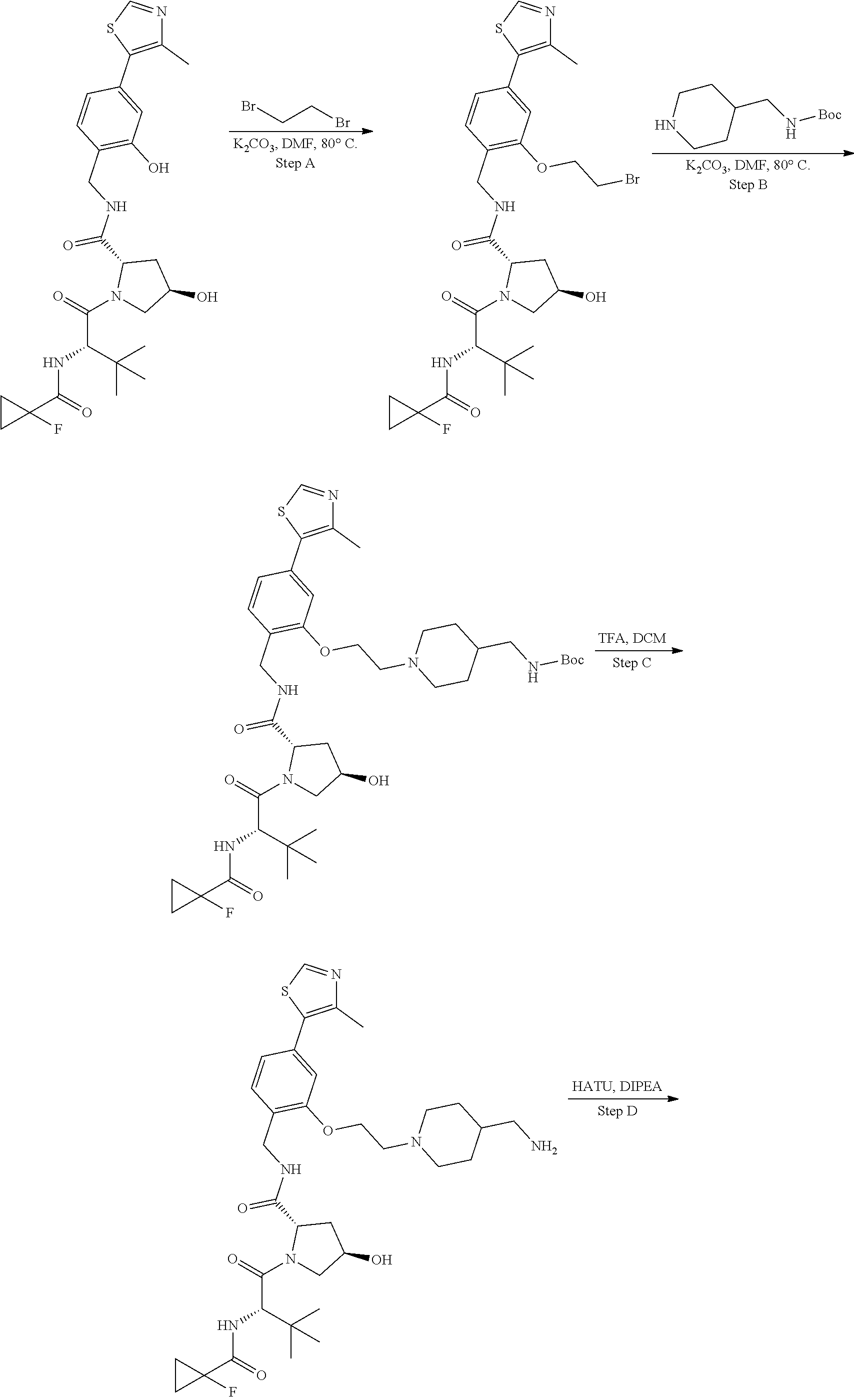

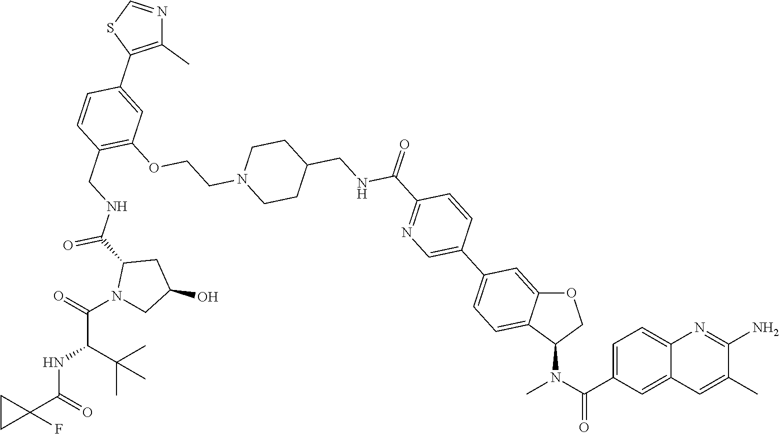 [SMILES: Cc1cc2cc(C(=O)N(C)[C@@H]3COc4cc(-c5ccc(C(=O)NCC6CCN(CCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N]

Example A109 was synthesized following the same procedures as described in Example A13 (5.9 mg, 2% yield over 4 steps) as a white solid. LC-MS (ESI): mass calcd. for C₆₀H₆₉FN₁₀O₈S, 1108.50; m/z found, 1109.7 [M+H]⁺. ¹H NMR (400 MHz, DMSO-d₆) δ 9.02-8.92 (m, 3H), 8.71 (s, 2H), 8.56-8.49 (m, 1H), 8.33-8.25 (m, 2H), 8.09 (d, J=8.2 Hz, 1H), 8.04 (s, 1H), 7.86 (d, J=8.1 Hz, 1H), 7.77 (s, 1H), 7.57 (d, J=7.6 Hz, 1H), 7.45-7.38 (m, 2H), 7.36 (s, 1H), 7.28-7.23 (m, 1H), 7.08-7.02 (m, 2H), 4.87-4.65 (m, 2H), 4.59-4.56 (m, 1H), 4.52-4.47 (m, 1H), 4.42-4.39 (m, 1H), 4.36-4.27 (m, 3H), 3.65-3.62 (m, 4H), 3.27 (s, 3H), 3.13-3.02 (m, 2H), 2.66 (s, 3H), 2.46 (s, 3H), 2.33 (s, 4H), 2.18-1.95 (m, 2H), 1.94-1.83 (m, 4H), 1.58-1.40 (m, 2H), 1.39-1.30 (m, 2H), 1.25-1.15 (m, 3H), 0.92 (s, 9H).

### Example A110. 4-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

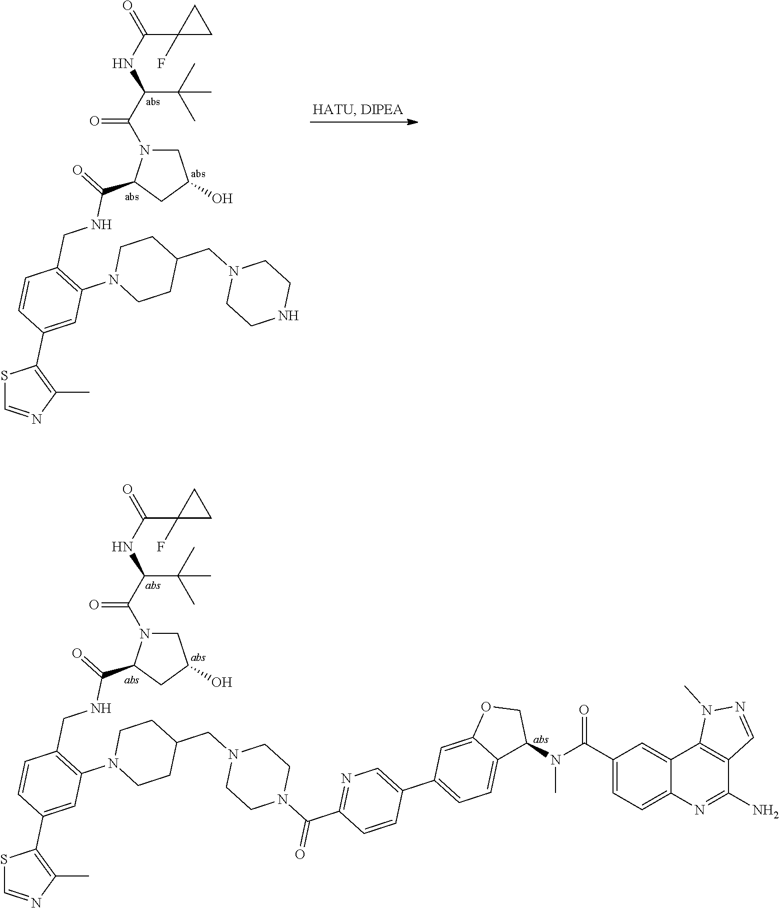 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCNCC3)CC2)c1 ; Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6nc(N)c7cnn(C)c7c6c5)cn4)CC3)CC2)c1]

Example A110 was synthesized following the same procedures as described in Example A107 (4.5 mg, 26% yield) as a white solid. LC-MS (ESI): mass calcd, for C₆₃H₇₂FN₁₃O₇S, 1173.5/z found, 1174.7 [M+H]⁺. ¹HNMR (400 MHz, DMSO-d₆) δ 9.64-9.49 (m, 1H), 8.99 (s, 1H), 8.94 (s, 1H), 8.64 (d, 1H), 8.51 (t, J=5.4 Hz, 1H), 8.46 (s, 1H), 8.28 (d, J=8.2 Hz, 1H), 7.93 (d, J=9.5 Hz, 1H), 7.88 (s, 1H), 7.78 (d, J=8.0 Hz, 1H), 7.61 (s, 1H), 7.47-7.39 (m, 2H), 7.36 (s, 1H), 7.27 (d, J=6.7 Hz, 1H), 7.15-7.11 (m, 2H), 4.79-4.73 (m, 1H), 4.60 (d, J=9.2 Hz, 1H), 4.54-4.47 (m, 4H), 4.38-4.34 (m, 2H), 4.32-4.23 (m, 1H), 3.69-3.59 (m, 4H), 3.29 (s, 3H), 3.21-3.07 (m, 8H), 2.75-2.64 (m, 6H), 2.46 (s, 3H), 2.11-2.07 (m, 1H), 2.02-1.96 (m, 1H), 1.95-1.90 (m, 1H), 1.89-1.84 (m, 2H), 1.50-1.42 (m, 2H), 1.41-1.38 (m, 1H), 1.37-1.33 (m, 1H), 1.24-1.23 (m, 1H), 1.22-1.19 (m, 1H), 0.97 (s, 9H).

### Example A111. 4-amino-N-((S)-6-(6-(4-((1-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-1,3-dihydrofuro[3,4-c][1,7]naphthyridine-8-carboxamide

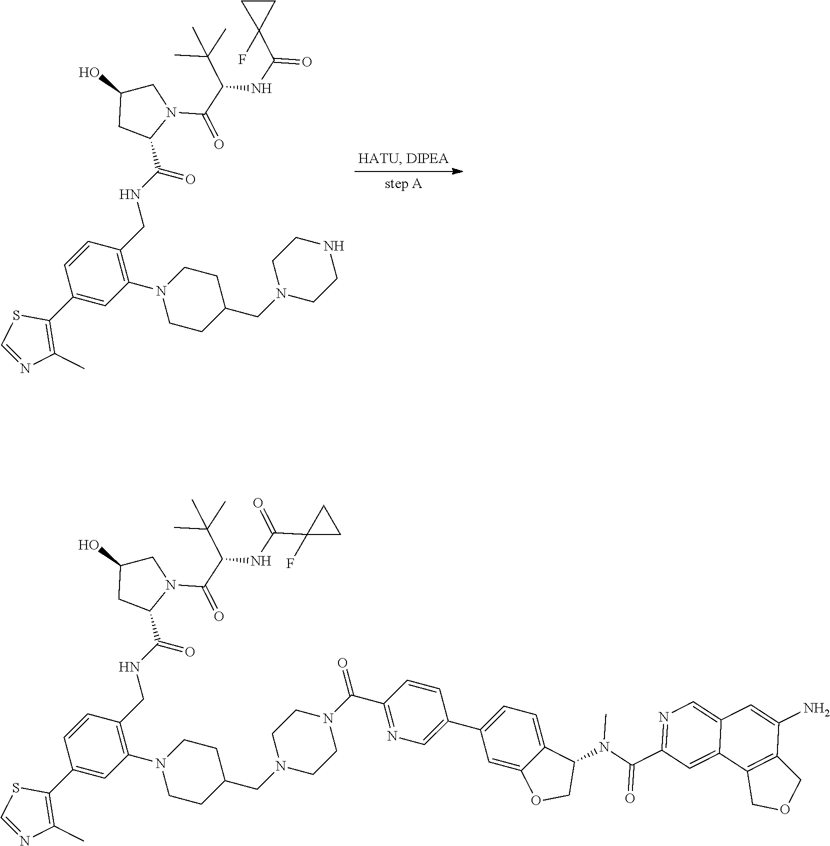 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCNCC3)CC2)c1 ; Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5cc6c7c(c(N)cc6cn5)COC7)cn4)CC3)CC2)c1]

Example A111 was synthesized following the same procedures as described in Example

A107 (3.5 mg, 14% yield) as a white solid. LC-MS (ESI): mass calcd, for C₆₂H₇₁FN₁₂O₈S, 1162.5/z found, 1164.6 [M+H]⁺. ¹HNMR (400 MHz, DMSO-d₆) δ 9.45 (s, 1H), 8.99 (s, 1H), 8.94 (s, 1H), 8.55-8.47 (m, 1H), 8.28 (d, J=8.0 Hz, 1H), 7.92 (d, J=19.9 Hz, 1H), 7.78 (d, J=8.3 Hz, 1H), 7.64 (d, J=8.4 Hz, 1H), 7.52-7.40 (m, 3H), 7.35 (d, J=10.7 Hz, 1H), 7.29-7.26 (m, 1H), 7.15-7.12 (m, 2H), 6.46-5.90 (m, 1H), 5.43 (s, 2H), 5.08 (s, 2H), 4.70-4.68 (m, 1H), 4.60 (d, J=9.3 Hz, 2H), 4.49 (t, 1H), 4.38-4.34 (m, 3H), 3.68-3.65 (m, 4H), 3.63-3.62 (m, 2H), 3.19-3.16 (m, 4H), 3.13-3.09 (m, 4H), 2.75-2.73 (m, 1H), 2.72 (s, 3H), 2.70-2.66 (m, 2H), 2.46 (s, 3H), 2.12-2.06 (m, 2H), 2.01-1.97 (m, 1H), 1.89-1.85 (m, 2H), 1.48-1.42 (m, 2H), 1.40-1.38 (m, 1H), 1.33 (s, 1H), 1.23-1.20 (m, 2H), 0.98 (s, 9H).

### Example A112. N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

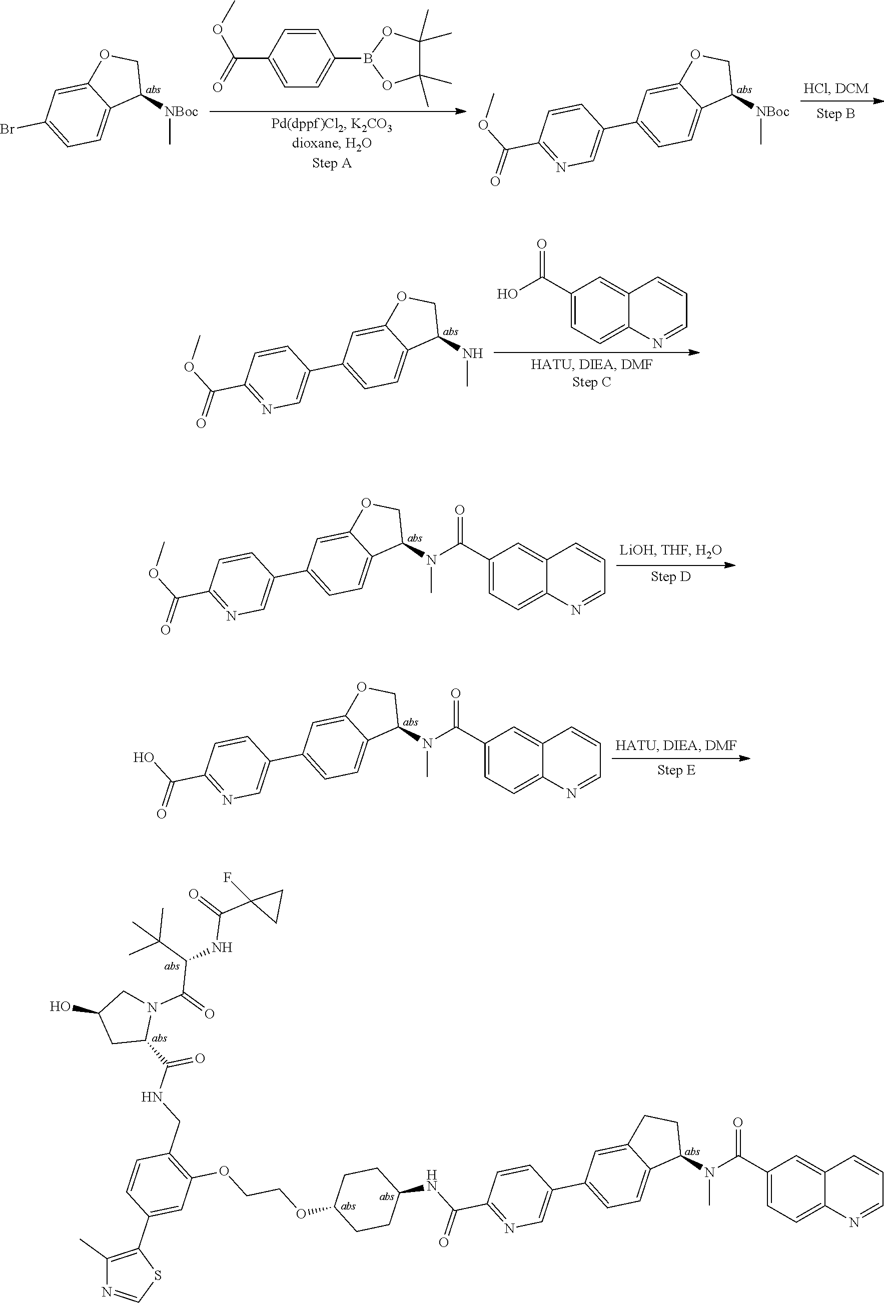

Example A112 was synthesized following the same procedures as described in Example A41 (10.9 mg, 5% yield over 5 steps) as a white solid. LC-MS (ESI): mass calcd. for C₅₉H₆₅FN₈O₉S, 1080.46; m/z found, 1081.6 [M+H]+. ¹HNMR (400 MHz, DMSO-d₆) δ 9.07 (dd, J=4.3, 1.5 Hz, 1H), 9.00 (s, 1H), 8.93 (s, 1H), 8.63 (s, 1H), 8.53-8.44 (m, 2H), 8.31-8.25 (m, 2H), 8.16 (s, 1H), 8.09 (d, J=8.2 Hz, 1H), 7.99-7.94 (m, 1H), 7.76-7.71 (m, 1H), 7.60 (d, J=7.7 Hz, 1H), 7.42 (d, J=7.8 Hz, 2H), 7.36 (s, 1H), 7.29 (dd, J=9.3, 2.6 Hz, 1H), 7.06 (d, J=1.4 Hz, 1H), 6.97 (dd, J=7.8, 1.4 Hz, 1H), 4.92-4.79 (m, 2H), 4.75-4.71 (m, 2H), 4.38-4.33 (m, 2H), 4.32-4.29 (m, 1H), 4.27-4.23 (m, 1H), 4.19-4.15 (m, 2H), 3.85-3.77 (m, 3H), 3.69-3.58 (m, 2H), 3.40-3.32 (m, 1H), 2.76-2.71 (m, 1H), 2.69-2.63 (m, 2H), 2.47 (s, 3H), 2.14-2.02 (m, 3H), 1.98-1.91 (m, 1H), 1.89-1.81 (m, 2H), 1.56-1.46 (m, 2H), 1.40-1.28 (m, 4H), 1.24-1.19 (m, 2H), 0.96 (s, 9H).

### Example A113. 4-amino-N-((S)-6-(6-(4-((1-(5-fluoro-2-(((2S,4R)-1-((S)-2-(1-fluorocyclopropane-1-carboxamido)-3,3-dimethylbutanoyl)-4-hydroxypyrrolidine-2-carboxamido)methyl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

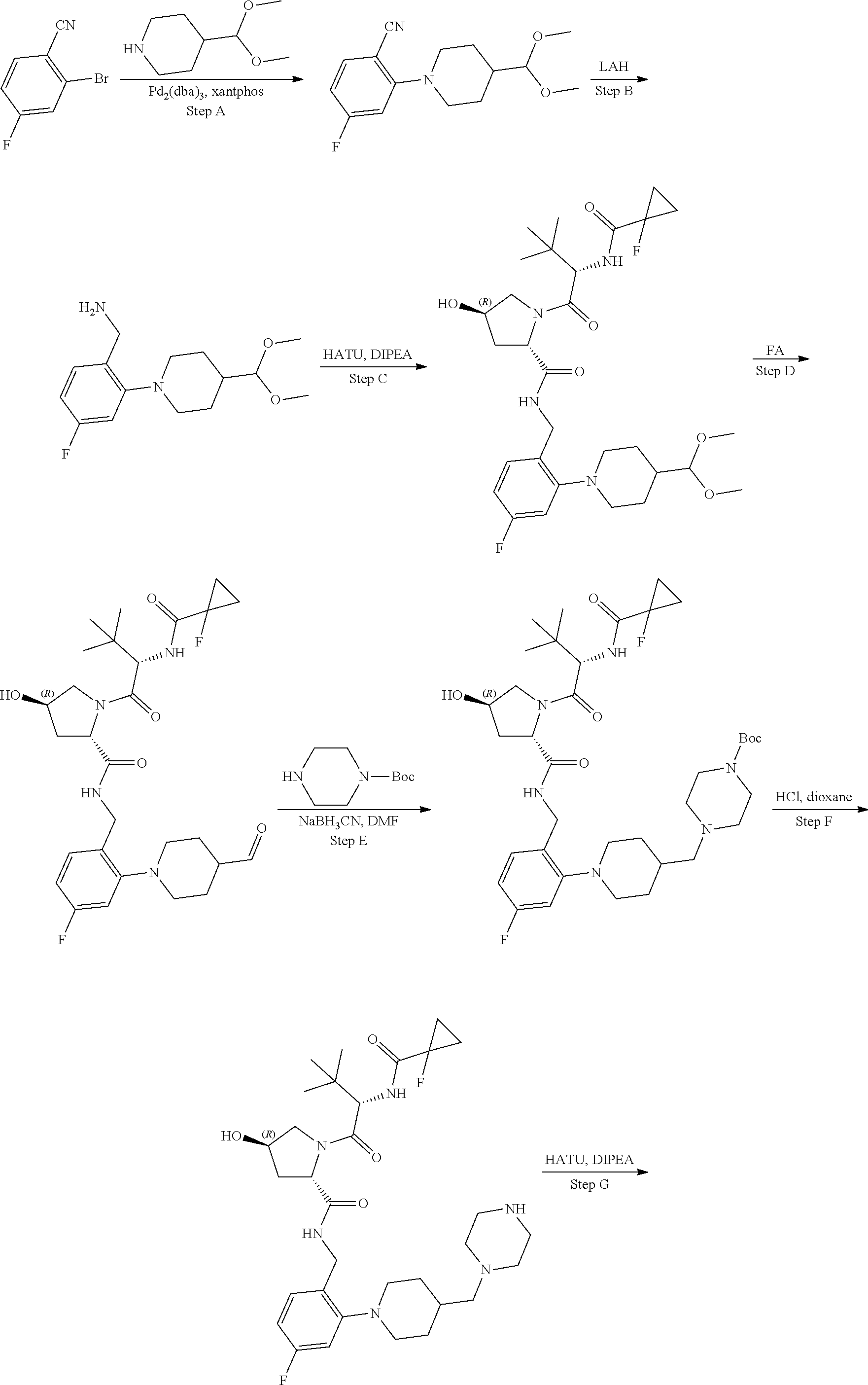

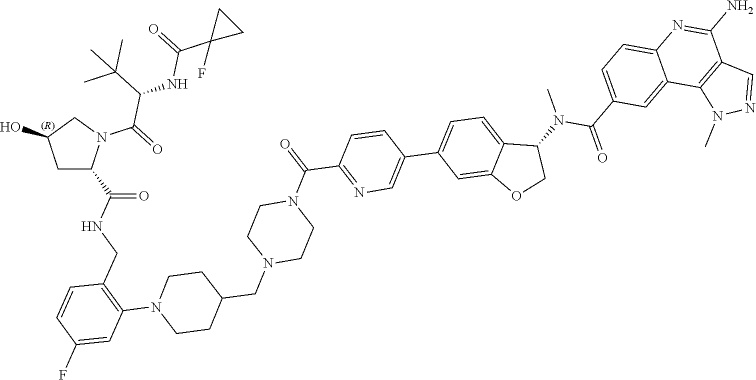

Step A: 2-(4-(dimethoxymethyl)piperidin-1-yl)-4-fluorobenzonitrile

To a solution of 2-bromo-4-fluorobenzonitrile (5 g, 25 mmol, 1.0 eq.) in 1,4-Dioxane (100 mL) was added 4-(dimethoxymethyl)piperidine (5.97 g, 37.5 mmol, 1.5 eq.), 4,5-Bis(diphenylphosphino)-9,9-dimethyl xanthene g, (2.89 g, 5 mmol, 0.2 eq.), Tris (dibenzylideneacetone) dipalladium (0)(1.14 g, 1.25 mmol, 0.1 eq.) and Cs₂CO₃ (24.4 g, 75 mmol, 3.0 eq.) at room temperature. The mixture was then stirred at 100° C. for 16 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~13% EtOAc in PE) to afford 2-(4-(dimethoxymethyl)piperidin-1-yl)-4-fluorobenzonitrile (1.27 g, 18% yield) as a yellow oil. LC-MS (ESI): mass calcd. For C₁₅H₁₉FN₂O₂, 278.1; m/z found, 279.0 [M+H]⁺.

Step B: (2-(4-(dimethoxymethyl)piperidin-1-yl)-4-fluorophenyl)methanamine

To a solution of 2-(4-(dimethoxymethyl)piperidin-1-yl)-4-fluorobenzonitrile (500 mg, 1.8 mmol, 1.0 eq.) in THF (5 mL) was added Aluminum lithium hydride (205 mg, 5.39 mmol, 3.0 eq.) at 0° C. The mixture was then stirred at room temperature for 16 hours under H₂ atmosphere. The solid was filtered out. The filtrate was concentrated under reduced pressure to afford (2-(4-(dimethoxymethyl)piperidin-1-yl)-4-fluorophenyl)methanamine (500 mg, 99% yield) as a yellow oil. LC-MS (ESI): mass calcd. For C₁₅H₂₃FN₂O₂, 282.2; m/z found, 283.2 [M+H]⁺.

Step C: (2S,4R)-N-(2-(4-(dimethoxymethyl)piperidin-1-yl)-4-fluorobenzyl)-1-((S)-2-(1-fluorocyclopropane-1-carboxamido)-3,3-dimethylbutanoyl)-4-hydroxypyrrolidine-2-carboxamide

To a solution of (2-(4-(dimethoxymethyl)piperidin-1-yl)-4-fluorophenyl)methanamine (250 mg, 885 μmol, 1.0 eq.) in DMF (3 mL) was added (2S,4R)-1-((S)-2-(1-fluorocyclopropane-1-carboxamido)-3,3-dimethylbutanoyl)-4-hydroxypyrrolidine-2-carboxylic acid (292 mg, 885 μmol, 1.0 eq.), HATU (673 mg, 1.77 mmol, 2.0 eq.) and DIEA (458 mg, 3.54 mmol, 4.0 eq.) at room temperature. The mixture was then stirred at room temperature for 1 hour under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~90% EtOAc in PE) to afford (2S,4R)-N-(2-(4-(dimethoxymethyl)piperidin-1-yl)-4-fluorobenzyl)-1-((S)-2-(1-fluorocyclopropane-1-carboxamido)-3,3-dimethylbutanoyl)-4-hydroxypyrrolidine-2-carboxamide (330 mg, 63% yield) as a white solid. LC-MS (ESI): mass calcd. For C₃₀H₄₄F₂N₄O₆, 594.3; m/z found, 595.8 [M+H]⁺.

Step D: (2S,4R)-N-(4-fluoro-2-(4-formylpiperidin-1-yl)benzyl)-1-((S)-2-(1-fluorocyclopropane-1-carboxamido)-3,3-dimethylbutanoyl)-4-hydroxypyrrolidine-2-carboxamide

To a solution of (2S,4R)-N-(2-(4-(dimethoxymethyl)piperidin-1-yl)-4-fluorobenzyl)-1-((S)-2-(1-fluorocyclopropane-1-carboxamido)-3,3-dimethylbutanoyl)-4-hydroxypyrrolidine-2-carboxamide (150 mg, 252 μmol, 1.0 eq.) in HCOOH (3 mL) was added at room temperature. The mixture was then stirred at room temperature for 1 hour under N₂ atmosphere. The reaction was concentrated under reduced pressure to afford (28,4R)-N-(4-fluoro-2-(4-formylpiperidin-1-yl)benzyl)-1-((S)-2-(1-fluorocyclopropane-1-carboxamido)-3,3-dimethylbutanoyl)-4-hydroxypyrrolidine-2-carboxamide (138 mg, 99% yield) as a yellow oil. LC-MS (ESI): mass calcd. For C28H38F₂N405, 548.3; m/z found, 549.5 [M+H]⁺.

Step E: tert-butyl 4-((1-(5-fluoro-2-(((2S,4R)-1-((S)-2-(1-fluorocyclopropane-1-carboxamido)-3,3-dimethylbutanoyl)-4-hydroxypyrrolidine-2-carboxamido)methyl)phenyl)piperidin-4-yl)methyl)piperazine-1-carboxylate

To a solution of (2S,4R)-N-(4-fluoro-2-(4-formylpiperidin-1-yl)benzyl)-1-((S)-2-(1-fluorocyclopropane-1-carboxamido)-3,3-dimethylbutanoyl)-4-hydroxypyrrolidine-2-carboxamide (92 mg, 168 μmol, 1.0 eq.) in DMF (3 mL) was added tert-butyl piperazine-1-carboxylate (31 mg, 168 μmol, 1.0 eq.) and AcOH (45 mg, 749 μmol, 4.5 eq.) at room temperature. The mixture was then stirred at room temperature for 2 hours under N₂. Sodium triacetoxyborohydride (142 mg, 671 μmol, 4.0 eq.) was added at room temperature. The mixture was then stirred at room temperature for 1 hour under N₂ atmosphere. The residue was purified Prep-HPLC (C18, 0~30%, MeCN in H₂O with 0.1% FA) to give tert-butyl 4-((1-(5-fluoro-2-(((2S,4R)-1-((S)-2-(1-fluorocyclopropane-1-carboxamido)-3,3-dimethylbutanoyl)-4-hydroxypyrrolidine-2-carboxamido)methyl)phenyl)piperidin-4-yl)methyl)piperazine-1-carboxylate (97 mg, 81% yield) as a white solid. LC-MS (ESI): mass calcd. For C37H56F₂N606, 718.4; m/z found, 719.6 [M+H]⁺.

Steps F-G: 4-amino-N-(S)-6-(6-(4-((1-(5-fluoro-2-((2S,4R)-1-((S)-2-(1-fluorocyclopropane-1-carboxamido)-3,3-dimethylbutanoyl)-4-hydroxypyrrolidine-2-carboxamido)methyl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

Example A113 was synthesized following the same procedures as described in Example A29 (10.4 mg, 1% yield over 2 steps) as a white solid. LC-MS (ESI): mass calcd. for C₅₉H68F₂N1207, 1094.5/z found, 1095.7 [M+H]⁺. ¹H NMR (400 MHz, DMSO-d₆) δ 9.57 (s, 2H), 8.96 (s, 1H), 8.94 (s, 1H), 8.64 (s, 1H), 8.46 (s, 2H), 8.28 (d, J=6.9 Hz, 1H), 7.94 (d, J=8.8 Hz, 1H), 7.87 (s, 1H), 7.78 (d, J=8.2 Hz, 1H), 7.61 (s, 1H), 7.40 (s, 1H), 7.38-7.32 (m, 2H), 7.29-7.23 (m, 1H), 6.91-6.86 (m, 1H), 6.85-6.79 (m, 1H), 4.77 (s, 1H), 4.60 (d, J=9.2 Hz, 1H), 4.52 (s, 3H), 4.48-4.44 (m, 1H), 4.35 (s, 1H), 4.30-4.23 (m, 2H), 3.68-3.66 (m, 2H), 3.66-3.64 (m, 2H), 3.63-3.61 (m, 2H), 3.60-3.57 (m, 2H), 3.56-3.46 (m, 3H), 3.35-3.29 (m, 1H), 3.16 (s, 3H), 3.08-3.03 (m, 2H), 2.74-2.59 (m, 5H), 2.10-2.03 (m, 1H), 2.00-1.93 (m, 1H), 1.91-1.85 (m, 2H), 1.47-1.32 (m, 4H), 1.25-1.20 (m, 2H), 0.96 (s, 9H).

### Example A114. 3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

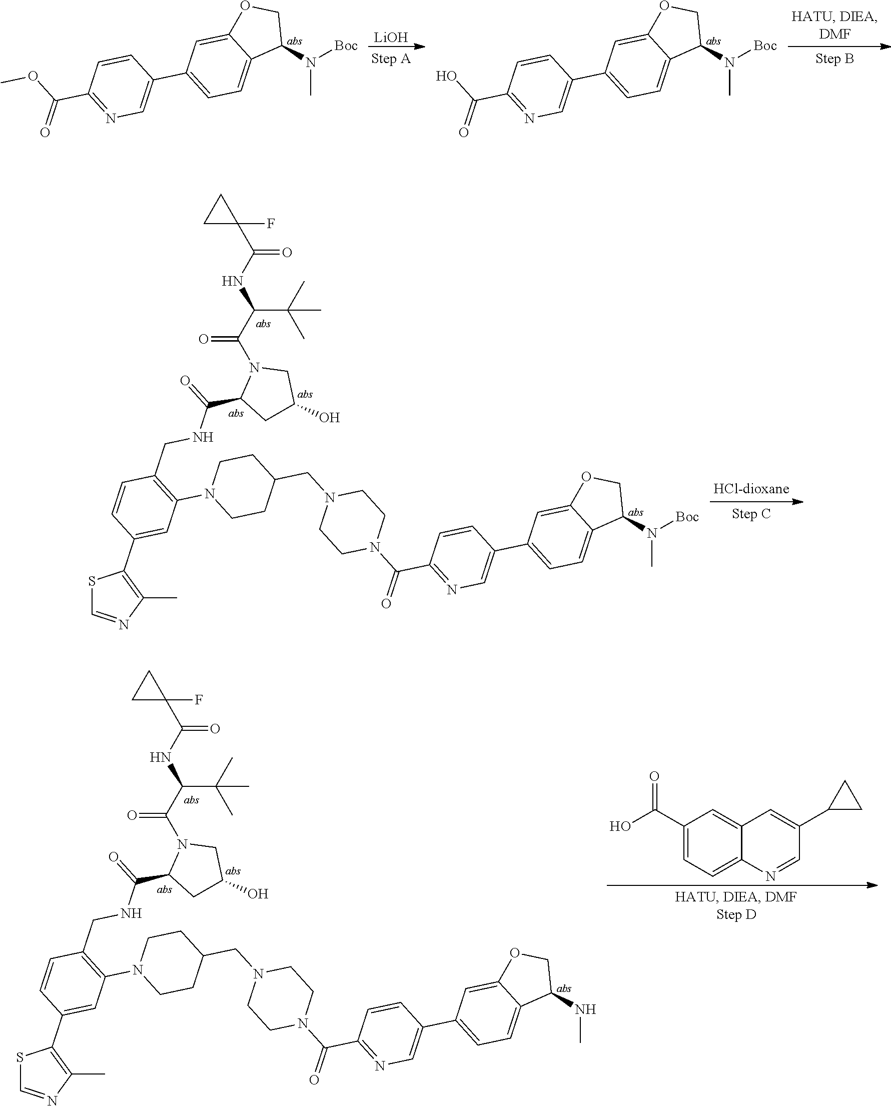

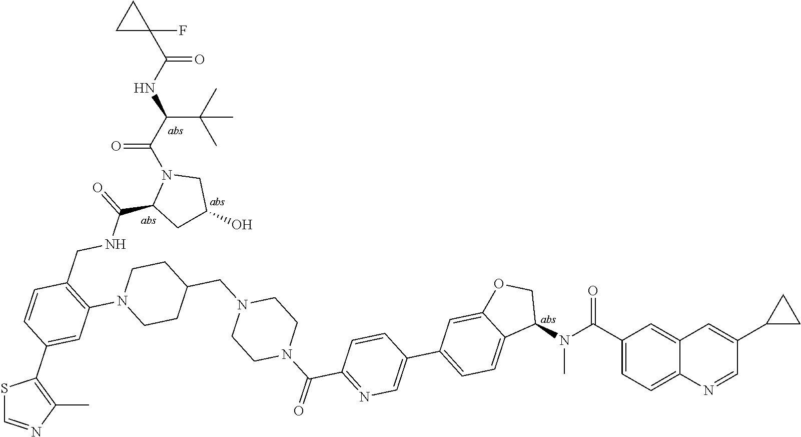 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncc(C7CC7)cc6c5)cn4)CC3)CC2)c1]

Step A: (S)-5-(3-((tert-butoxycarbonyl)(methyl)amino)-2,3-dihydrobenzofuran-6-yl)picolinic acid

A solution of methyl(S)-5-(3-((tert-butoxycarbonyl)(methyl)amino)-2,3-dihydrobenzofuran-6-yl)picolinate (1.20 g, 3.12 mmol, 1.0 eq.), LiOH (112 mg, 4.68 mmol, 1.5 eq.) in THF (10 mL) and Water (10 mL). The mixture was stirred at room temperature for 2 hours. The mixture was concentrated to afford(S)-5-(3-((tert-butoxycarbonyl)(methyl)amino)-2,3-dihydrobenzofuran-6-yl)picolinic acid (1.10 g, 95% yield) as a white solid. LC-MS (ESI): mass calcd. For C₂₀H₂₂N₂O₅, 370.15; m/z found, 371.1 [M+H]⁺

Step B: tert-butyl ((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)(methyl)carbamate

To a mixture of (2S,4R)-1-((S)-2-(1-fluorocyclopropane-1-carboxamido)-3,3-dimethylbutanoyl)-4-hydroxy-N-(4-(4-methylthiazol-5-yl)-2-(4-(piperazin-1-ylmethyl)piperidin-1-yl)benzyl)pyrrolidine-2-carboxamide (300 mg, 430 μmol, 1.0 eq.) in DMF (10 mL), was added(S)-5-(3-((tert-butoxycarbonyl)(methyl)amino)-2,3-dihydrobenzofuran-6-yl)picolinic acid (159 mg, 430 μmol, 1.0 eq.), HATU (245 mg, 645 μmol, 1.5 eq.) and DIEA (278 mg, 374 μL, 2.15 mmol, 5.0 eq.). The resulting mixture was stirred at room temperature for 2 hours. The reaction mixture was poured into water (50 mL), and then extracted with EtOAc (50 mL×2). The combined organic layers were washed with brine, dried over anhydrous Na₂SO₄ and concentrated. The residue was purified by column chromatography (silica gel, 0-8% MeOH in DCM) to afford tert-butyl ((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)(methyl)carbamate (150 mg, 33% yield) as a yellow solid. LC-MS (ESI): mass calcd. For C₅₆H₇₂FN₉O₈S, 1049.52; m/z found, 1050.4 [M+H]⁺

Step C: (2S, 4R)-1-((S)-2-(1-fluorocyclopropane-1-carboxamido)-3,3-dimethylbutanoyl)-4-hydroxy-N-(2-(4-((4-(5-((S)-3-(methylamino)-2,3-dihydrobenzofuran-6-yl)picolinoyl)piperazin-1-yl)methyl)piperidin-1-yl)-4-(4-methylthiazol-5-yl)benzyl)pyrrolidine-2-carboxamide

To a solution of tert-butyl ((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)(methyl)carbamate (150 mg, 143 μmol, 1.0 eq.) in DCM (2 mL) was added hydrochloric acid (2 mL, 4 M/L), the reaction mixture was stirred at room temperature for 2 hours. The reaction mixture was concentrated under reduced pressure to afford (2S,4R)-1-((S)-2-(1-fluorocyclopropane-1-carboxamido)-3,3-dimethylbutanoyl)-4-hydroxy-N-(2-(4-((4-(5-((S)-3-(methylamino)-2,3-dihydrobenzofuran-6-yl)picolinoyl)piperazin-1-yl)methyl)piperidin-1-yl)-4-(4-methylthiazol-5-yl)benzyl)pyrrolidine-2-carboxamide (130 mg, 95% yield) as a yellow solid.

LC-MS (ESI): mass calcd. For C₅₁H₆₄FN₉O₆S, 949.47; m/z found, 950.3 [M+H]⁺

Step D: 3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

Example A114 was synthesized following the same procedures as described in Example A107 (6.7 mg, 37% yield over 1 step) as a white solid. LC-MS (ESI): mass calcd. for C₆₄H₇₃FN₁₀O₇S, 1144.5/z found, 1145.7 [M+H]⁺. ¹HNMR (400 MHz, DMSO-d₆) δ 9.00-8.96 (m, 1H), 8.91 (s, 1H), 8.83 (d, J=2.1 Hz, 1H), 8.49 (t, 1H), 8.22 (d, J=7.0 Hz, 1H), 8.05 (s, 3H), 7.77 (d, J=8.7 Hz, 1H), 7.65 (d, J=8.1 Hz, 1H), 7.57 (d, J=7.4 Hz, 1H), 7.44-7.37 (m, 2H), 7.34 (s, 1H), 7.28 (dd, J=6.5 Hz, 1H), 7.12-7.08 (m, 2H), 6.03 (d, J=327.9 Hz, 1H), 5.17 (d, J=3.6 Hz, 1H), 4.88-4.66 (m, 2H), 4.60 (d, J=9.4 Hz, 1H), 4.48 (t, J=8.2 Hz, 1H), 4.40-4.26 (m, 3H), 3.72-3.63 (m, 3H), 3.62-3.58 (m, 1H), 3.50-3.45 (m, 2H), 3.39-3.35 (m, 1H), 3.29 (s, 3H), 3.08-3.03 (m, 2H), 2.73-2.61 (m, 6H), 2.45 (s, 3H), 2.38 (s, 2H), 2.34-2.31 (m, 1H), 2.24 (d, 2H), 2.20-2.16 (m, 1H), 2.11-2.05 (m, 1H), 1.97-1.90 (m, 1H), 1.83 (d, J=11.6 Hz, 2H), 1.68 (s, 1H), 1.40-1.29 (m, 4H), 1.26-1.18 (m, 3H), 1.11 (dd, J=8.3, 2.0 Hz, 2H), 0.98 (s, 9H), 0.90 (d, J=3.7 Hz, 2H).

### Example A115. N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

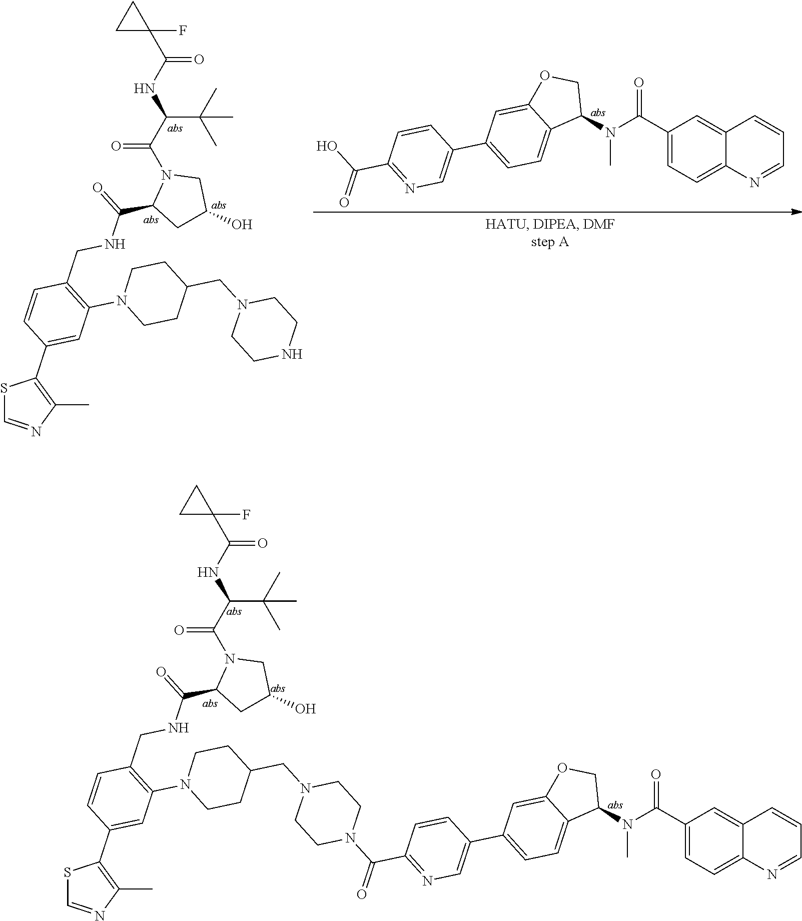 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCNCC3)CC2)c1 ; CN(C(=O)c1ccc2ncccc2c1)[C@@H]1COc2cc(-c3ccc(C(=O)O)nc3)ccc21 ; Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncccc6c5)cn4)CC3)CC2)c1]

Example A115 was synthesized following the same procedures as described in Example A107 (7.0 mg, 37% yield) as a yellow solid. LC-MS (ESI): mass calcd, for C₆₁H₆₉FN₁₀O₇S, 1104.5/z found, 1105.6 [M+H]⁺. ¹HNMR (400 MHz, DMSO-d₆) δ 9.65 (s, 1H), 9.03-8.96 (m, 2H), 8.94 (s, 1H), 8.52 (t, J=5.7 Hz, 2H), 8.29 (d, J=7.8 Hz, 1H), 8.20 (s, 1H), 8.13 (s, 1H), 7.90 (d, J=8.4 Hz, 1H), 7.78 (d, J=8.2 Hz, 1H), 7.67-7.57 (m, 2H), 7.48-7.39 (m, 2H), 7.36 (s, 1H), 7.27 (d, J=6.6 Hz, 1H), 7.16-7.10 (m, 2H), 4.92-4.70 (m, 2H), 4.60 (d, J=8.8 Hz, 2H), 4.49 (t, J=8.2 Hz, 1H), 4.38-4.34 (m, 2H), 4.28-4.20 (m, 1H), 3.68-3.58 (m, 6H), 3.29 (s, 2H), 3.18-3.15 (m, 2H), 3.12-3.08 (m, 2H), 2.78-2.65 (m, 6H), 2.46 (s, 3H), 2.12-2.07 (m, 1H), 2.01-1.96 (m, 1H), 1.91-1.86 (m, 2H), 1.51-1.42 (m, 2H), 1.41-1.38 (m, 1H), 1.36-1.33 (m, 1H), 1.25-1.19 (m, 3H), 0.98 (s, 9H).

### Example A116. 2-amino-N-((S)-6-(6-(((1-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)piperidin-4-yl)methyl)carbamoyl)pyridin-3-yl)-2,3-dihydrobenzofuran-3-yl)-N-isopropyl-3-methylquinoline-6-carboxamide

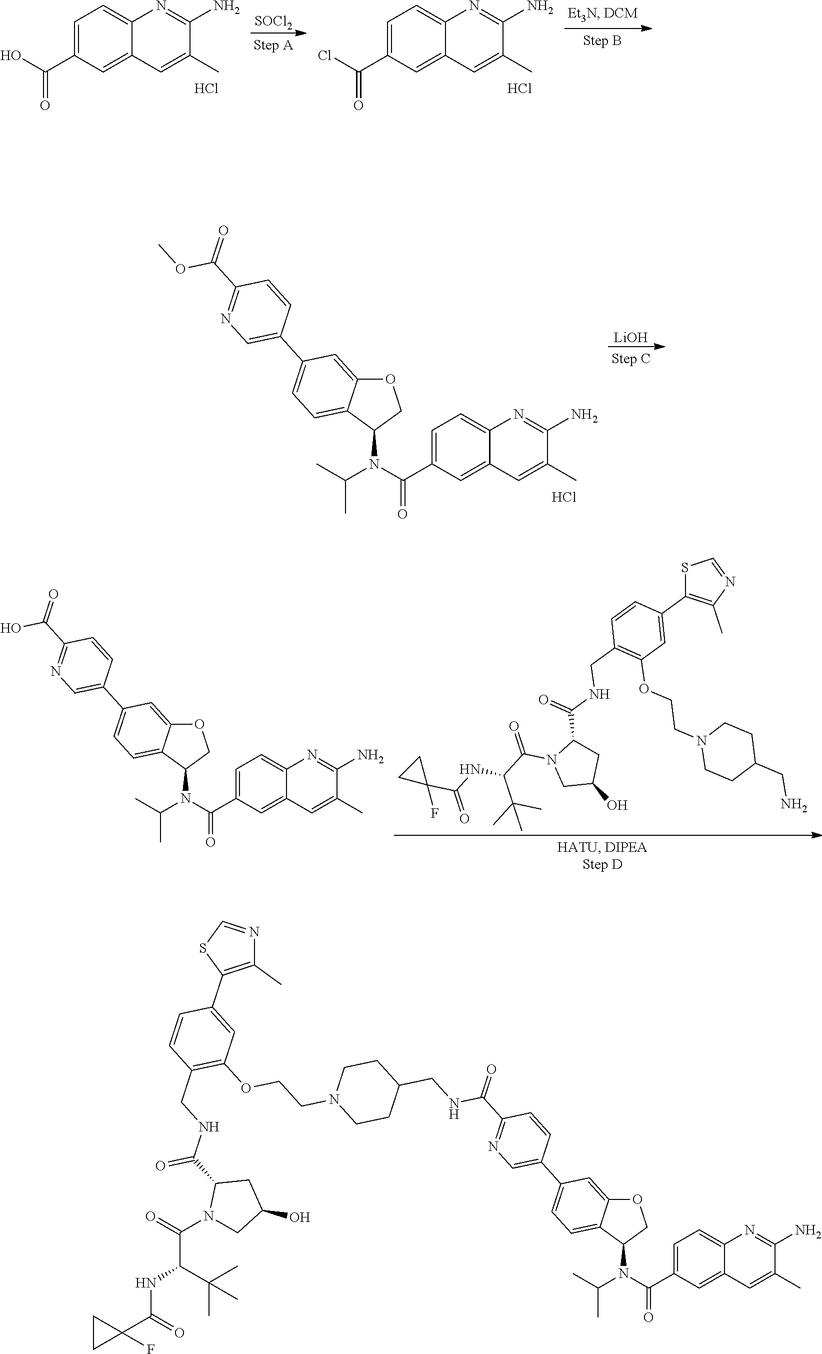

Example A116 was synthesized following the same procedures as described in Example A45 (8.5 mg, 3% yield over 4 steps) as a white solid. LC-MS (ESI): mass calcd. for C₆₂H₇₃FN₁₀O₈S, 1136.53; m/z found, 1138 [M+H]⁺. ¹H NMR (400 MHz, DMSO-d₆) δ 9.46-9.18 (m, 1H), 9.02-8.95 (m, 2H), 8.91 (s, 1H), 8.89-8.58 (m, 2H), 8.57-8.50 (m, 1H), 8.30 (s, 1H), 8.25 (d, J=7.8 Hz, 1H), 8.08 (d, J=8.2 Hz, 1H), 7.88 (s, 1H), 7.71 (s, 2H), 7.44 (d, J=7.8 Hz, 1H), 7.42-7.33 (m, 1H), 7.31-7.25 (m, 2H), 7.22 (s, 1H), 7.08-7.02 (m, 2H), 5.45-5.36 (m, 1H), 4.79-4.66 (m, 2H), 4.58 (d, J=9.1 Hz, 1H), 4.52-4.47 (m, 1H), 4.45-4.38 (m, 2H), 4.33 (d, J=14.7 Hz, 2H), 4.31-4.29 (m, 1H), 3.89-3.82 (m, 1H), 3.68-3.61 (m, 6H), 3.30-3.25 (m, 2H), 3.13-3.03 (m, 2H), 2.47 (s, 3H), 2.31 (s, 3H), 2.11-2.06 (m, 1H), 1.95-1.88 (m, 3H), 1.86-1.70 (m, 1H), 1.56-1.45 (m, 2H), 1.41-1.38 (m, 2H), 1.37-1.30 (m, 2H), 1.29-1.09 (m, 6H), 0.93 (s, 9H).

### Example A117. 4-amino-N-((S)-6-(6-(6-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,6-diazaspiro[3.3]heptane-2-carbonyl)pyridin-3-yl)-2,3-dihydrobenzofuran-3-yl)-N,1-dimethyl-1H-pyrazolo[4,3-c]quinoline-8-carboxamide

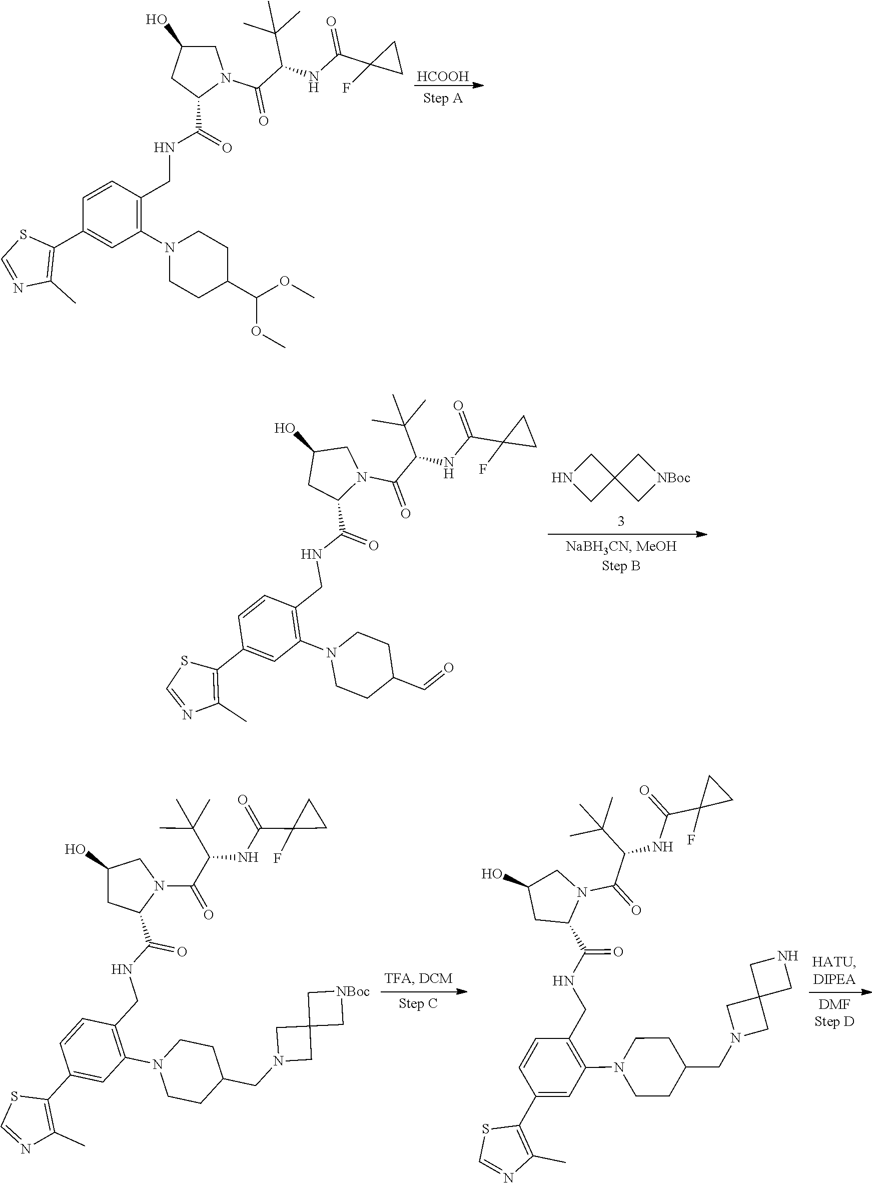

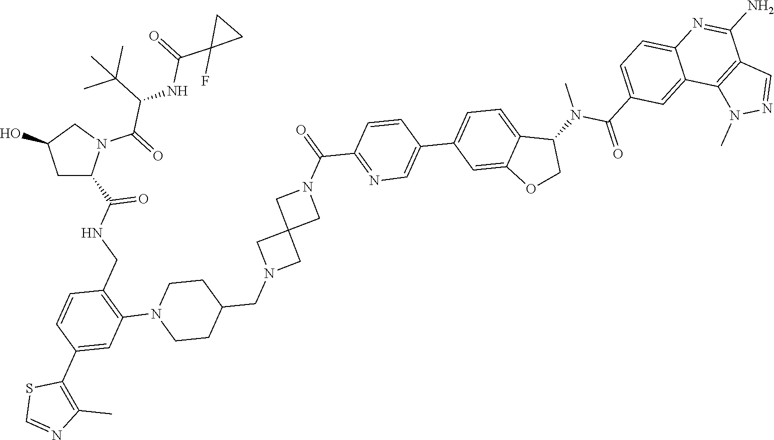 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CC4(C3)CN(C(=O)c3ccc(-c5ccc6c(c5)OC[C@H]6N(C)C(=O)c5ccc6nc(N)c7cnn(C)c7c6c5)cn3)C4)CC2)c1]

Example A117 was synthesized following the same procedures as described in Example A107 (1.4 mg, 0.9% yield over 4 steps) as a white solid. LC-MS (ESI): mass calcd. for C₆₄H₇₂FN₁₃O₇S, 1185.54; m/z found, 1186.8 [M+H]⁺. ¹HNMR (400 MHz, DMSO-d₆) δ 9.59 (s, 1H), 9.02-8.90 (m, 2H), 8.83 (s, 1H), 8.64 (s, 1H), 8.54-8.39 (m, 2H), 8.27 (d, J=7.9 Hz, 1H), 8.03 (d, J=8.0 Hz, 1H), 7.96-7.83 (m, 2H), 7.61 (s, 1H), 7.46-7.32 (m, 3H), 7.27 (d, J=8.9 Hz, 1H), 7.18-7.02 (m, 2H), 4.90-4.86 (m, 1H), 4.80-4.74 (m, 2H), 4.62-4.58 (m, 1H), 4.53-4.47 (m, 4H), 4.44-4.39 (m, 2H), 4.37-4.25 (m, 6H), 4.24-4.20 (m, 1H), 3.68-3.60 (m, 4H), 3.17-3.06 (m, 4H), 2.71-2.69 (m, 2H), 2.46 (s, 3H), 2.12-2.05 (m, 1H), 1.95-1.88 (m, 1H), 1.79-1.67 (m, 3H), 1.44-1.33 (m, 4H), 1.24-1.20 (m, 2H), 1.03-0.94 (m, 9H).

### Example A118. N1-(7-amino-1-methyl-1H-pyrazolo[3,4-c]pyridin-4-yl)-N2-((S)-6-(6-(((1S,4S)-4-(2-(2-(((2S,4S)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N2-methyloxalamide

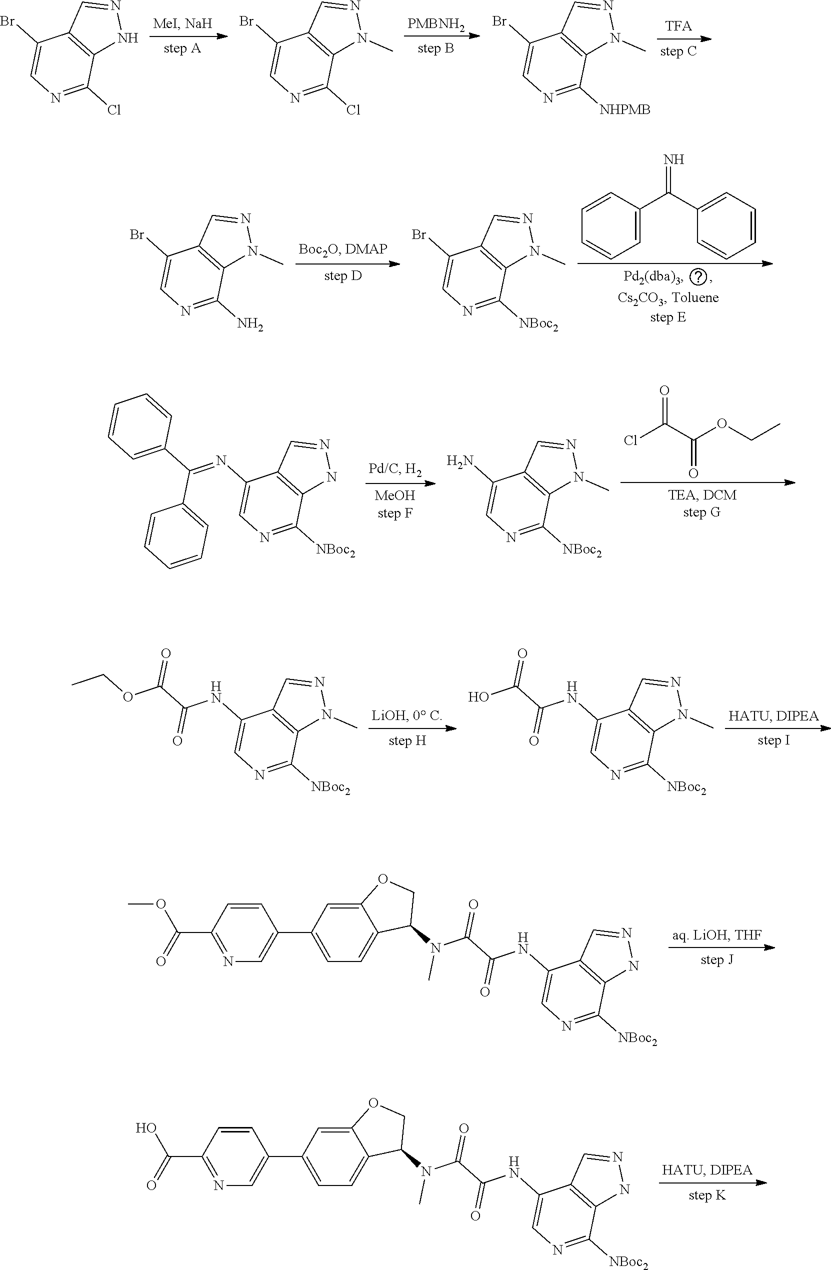

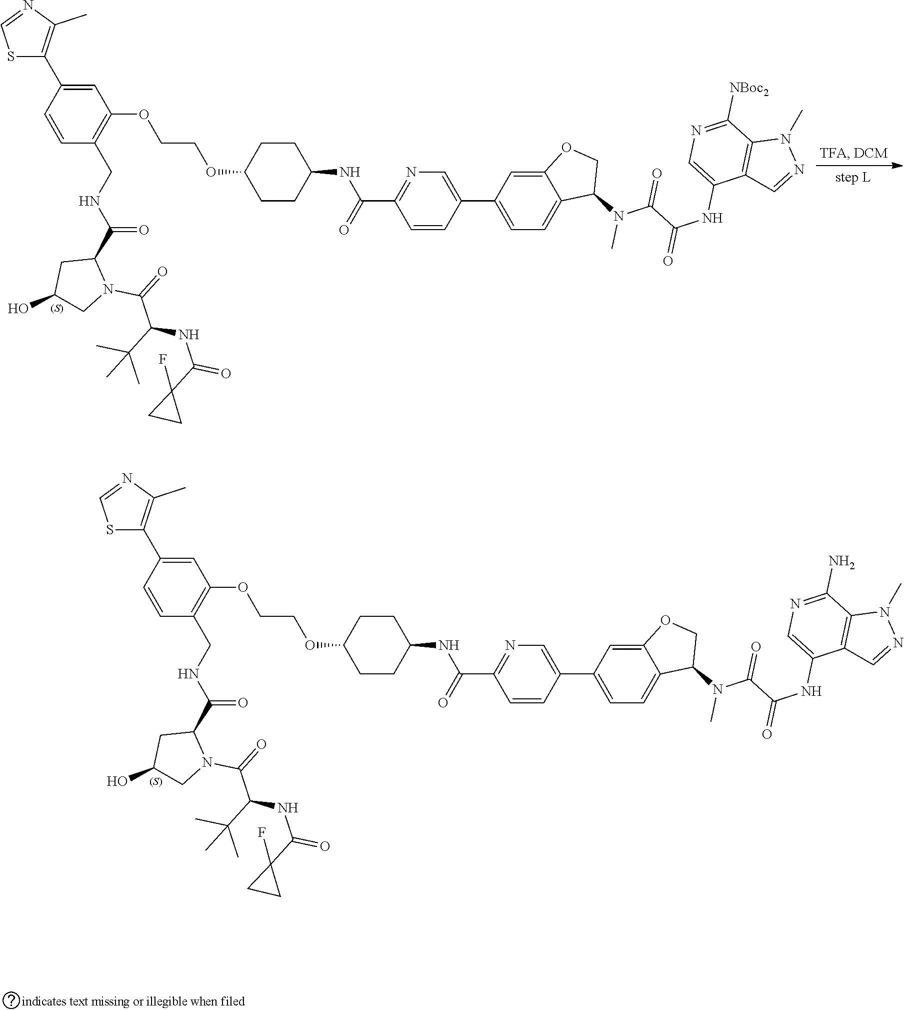

Example A118 was synthesized following the same procedures as described in Example A44 (6.3 mg, 0.5% yield over 12 steps) as a white solid. LC-MS (ESI): mass calcd. for C₅₈H₆₇FN₁₂O₁₀S, 1142.5/z found, 1144.6 [M+H]⁺. ¹HNMR (400 MHz, DMSO-d₆) δ 10.80 (d, J=63.1 Hz, 1H), 8.99 (s, 1H), 8.93 (s, 1H), 8.57 (t, J=5.8 Hz, 1H), 8.49 (d, J=7.9 Hz, 1H), 8.30-8.24 (m, 1H), 8.10-7.85 (m, 3H), 7.55 (dd, J=51.1, 8.0 Hz, 1H), 7.44-7.35 (m, 3H), 7.31 (dd, J=8.5, 1.6 Hz, 1H), 7.06 (s, 1H), 6.96 (d, J=7.8 Hz, 1H), 6.50 (s, 1H), 6.33-5.80 (m, 1H), 5.46 (d, J=7.7 Hz, 1H), 4.84-4.74 (m, 1H), 4.71-4.62 (m, 1H), 4.55 (d, J=8.9 Hz, 1H), 4.47-4.42 (m, 1H), 4.38-4.32 (m, 1H), 4.29 (s, 3H), 4.27-4.22 (m, 2H), 4.18 (s, 2H), 3.88-3.78 (m, 4H), 3.50-3.44 (m, 3H), 2.77-2.64 (m, 3H), 2.47 (s, 3H), 2.39-2.33 (m, 1H), 2.08-2.03 (m, 2H), 1.89-1.83 (m, 2H), 1.81-1.74 (m, 1H), 1.56-1.47 (m, 2H), 1.33 (t, J=15.9 Hz, 4H), 1.24-1.19 (m, 2H), 0.97 (s, 9H).

### Example A119. (2S,4R)-N-(2-(4-((2-(4-amino-1-methyl-1H-pyrazolo[4,3-c]quinoline-8-carbonyl)-2,7-diazaspiro[3.5]nonan-7-yl)methyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

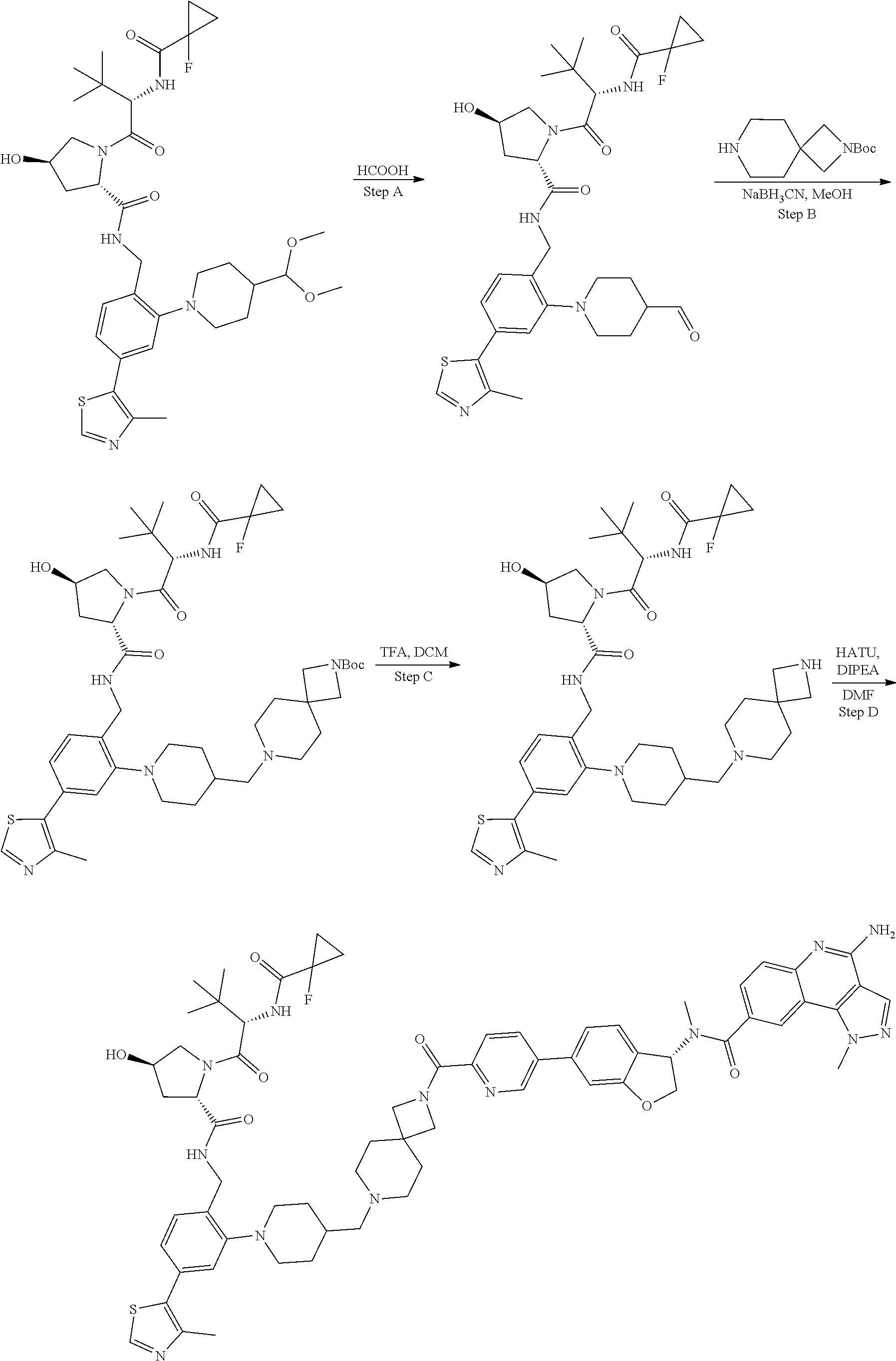

Step A: (2S,4R)-1-((S)-2-(1-fluorocyclopropane-1-carboxamido)-3,3-dimethylbutanoyl)-N-(2-(4-formylpiperidin-1-yl)-4-(4-methylthiazol-5-yl)benzyl)-4-hydroxypyrrolidine-2-carboxamide

To a solution of (2S,4R)-N-(2-(4-(dimethoxymethyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (100 mg, 148 μmol, 1.0 eq.) in MeCN (20 mL) was added formic acid (68 mg, 1.48 mol, 10 eq.) at room temperature. The mixture was stirred for 1 hour at 55° C. The mixture was concentrated by vacuum to afford (2S,4R)-1-((S)-2-(1-fluorocyclopropane-1-carboxamido)-3,3-dimethylbutanoyl)-N-(2-(4-formylpiperidin-1-yl)-4-(4-methylthiazol-5-yl)benzyl)-4-hydroxypyrrolidine-2-carboxamide (5.00 mg, 54% yield) as a solid.

LC-MS (ESI): mass calcd. For C₃₂H₄₂FN₅O₅S, 627.29; m/z found, 628 [M+H]⁺.

Step B: tert-butyl 7-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,7-diazaspiro[3.5]nonane-2-carboxylate

A mixture of (2S,4R)-1-((S)-2-(1-fluorocyclopropane-1-carboxamido)-3,3-dimethylbutanoyl)-N-(2-(4-formylpiperidin-1-yl)-4-(4-methylthiazol-5-yl)benzyl)-4-hydroxypyrrolidine-2-carboxamide (50 mg, 79.6 μmol, 1.0 eq.), tert-butyl 2,7-diazaspiro[3.5]nonane-2-carboxylate (22 mg, 95.6 μmol, 1.2 eq.) and HOAc (3 mg, 39.8 μmol, 0.5 eq.) in MeOH (3 mL) was stirred at room temperature for 1 hour under nitrogen. Then NaBH₃CN (10 mg, 159 μmol, 2.0 eq.) was added at room temperature and stirred for 1 hour. After cooling to room temperature, the resulting solution was diluted with 50 mL of water and extracted with EtOAc (30 mL×3). The combined organic layers were washed with brine, dried over Na₂SO₄ and concentrated under reduced pressure. The crude product was purified by flash column chromatography on silica gel (100-200 mesh silica gel, 0~10% MeOH in DCM) to afford tert-butyl 7-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,7-diazaspiro[3.5]nonane-2-carboxylate (30 mg, 45% yield) as a white solid. LC-MS (ESI): mass calcd. For C₄₄H₆₄FN₇O₆s, 837.46; m/z found, 838 [M+H]⁺.

Step C: (2S, 4R)-N-(2-(4-((2,7-diazaspiro[3.5]nonan-7-yl)methyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of tert-butyl 7-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,7-diazaspiro[3.5]nonane-2-carboxylate (30 mg, 35.8 μmol, 1.0 eq.) in DCM (3 mL) was added TFA (1 mL). The mixture was stirred at room temperature for 30 mins under nitrogen. The resulting solution was removed under reduced pressure to afford (2S,4R)-N-(2-(4-((2,7-diazaspiro[3.5]nonan-7-yl)methyl)piperidin-1-yl)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (25.0 mg, 94% crude) as a yellow solid.

LC-MS (ESI): mass calcd. For C₃₉H₅₆FN₇O₄S, 737.41; m/z found, 738 [M+H]⁺.

Step D: (2S,4R)-N-(2-(4-((2-(4-amino-1-methyl-1H-pyrazolo[4,3-c]quinoline-8-carbonyl)-2,7-diazaspiro[3.5]nonan-7-yl)methyl)piperidin-1-yl)-4-(4-methylthiazol-5-yl)benzyl)-1-((S)-2-(1-fluorocyclopropane-1-carboxamido)-3, 3-dimethylbutanoyl)-4-hydroxypyrrolidine-2-carboxamide

Example A119 was synthesized following the same procedures as described in Example A107 (2.2 mg, 3% yield) as a white solid. LC-MS (ESI): mass calcd, for C66H76FN₁₃O₇S, 1213.57; m/z found, 1214.7 [M+H]⁺. ¹H NMR (400 MHz, DMSO-d₆) δ 9.69 (s, 1H), 9.03-8.97 (m, 2H), 8.93-8.88 (m, 1H), 8.63 (s, 1H), 8.56-8.39 (m, 2H), 8.29-8.21 (m, 1H), 8.09-8.00 (m, 1H), 7.96-7.73 (m, 2H), 7.67-7.55 (m, 1H), 7.47-7.42 (m, 1H), 7.40 (s, 1H), 7.36-7.31 (m, 1H), 7.27 (d, J=7.6 Hz, 1H), 7.17-7.08 (m, 2H), 4.88-4.75 (m, 2H), 4.72-4.64 (m, 1H), 4.62-4.59 (m, 1H), 4.52-4.49 (m, 3H), 4.41-4.37 (m, 2H), 3.95-3.94 (m, 2H), 3.64-3.61 (m, 4H), 3.50-3.45 (m, 3H), 3.09-3.01 (m, 4H), 2.72-2.69 (m, 2H), 2.46 (s, 3H), 2.14-2.10 (m, 1H), 1.99-1.91 (m, 3H), 1.86-1.83 (m, 1H), 1.73-1.71 (m, 2H), 1.49-1.47 (m, 6H), 1.36-1.35 (m, 2H), 1.00-0.95 (m, 9H).

### Example A120. 4-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-isopropyl-1,3-dihydrofuro[3,4-c][1,7]naphthyridine-8-carboxamide

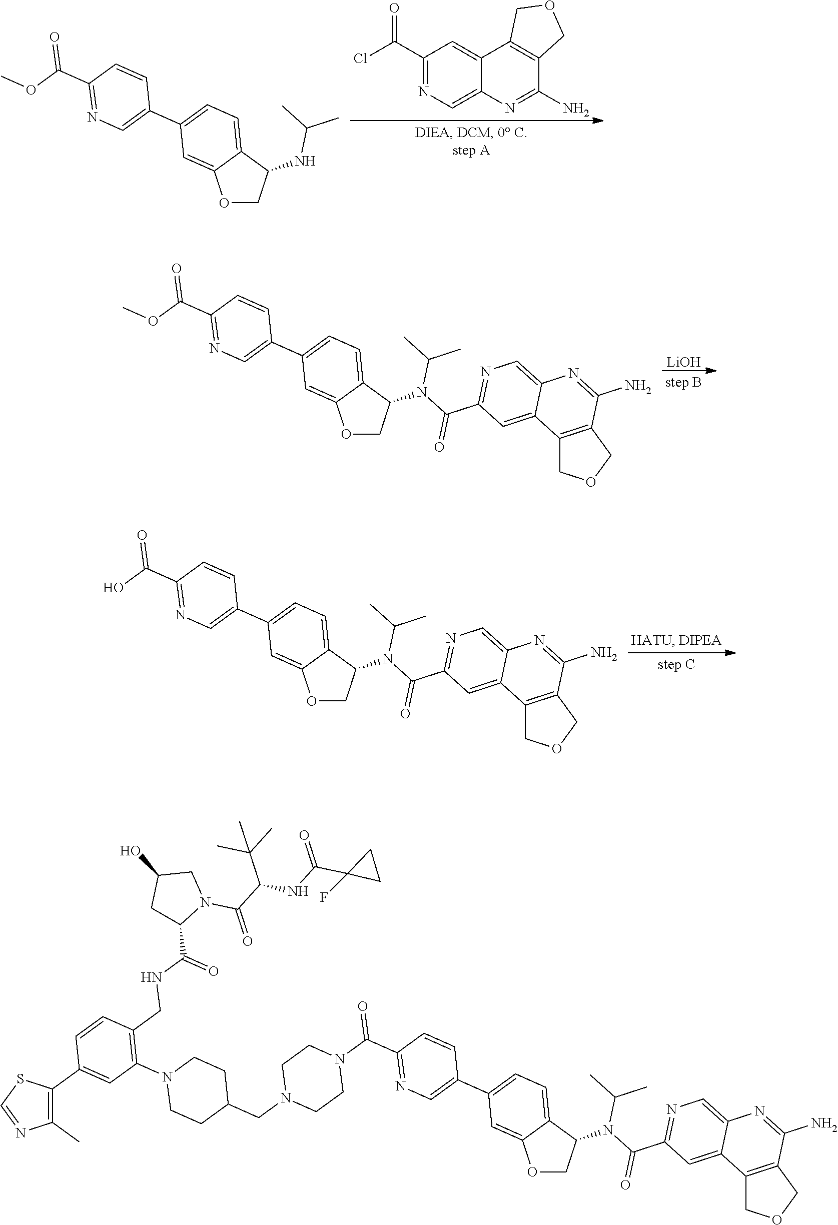

Example A120 was synthesized following the same procedures as described in Example A45 (2.5 mg, 3% yield over 3 steps) as a white solid. LC-MS (ESI): mass calcd. for C₆₄H₇₅FN₁₂O₈S, 1190.6/z found, 1191.9 [M+H]⁺. ¹HNMR (400 MHz, DMSO-d₆) δ 9.61-9.29 (m, 1H), 8.99 (s, 1H), 8.93-8.86 (m, 2H), 8.51 (t, J=5.7 Hz, 1H), 8.24 (d, J=9.1 Hz, 1H), 7.78 (d, J=8.0 Hz, 1H), 7.69 (s, 1H), 7.46-7.43 (m, 1H), 7.32-7.24 (m, 3H), 7.20 (s, 1H), 7.15-7.12 (m, 2H), 5.44-5.39 (m, 1H), 5.35 (s, 2H), 5.05 (s, 2H), 4.79 (t, J=9.6 Hz, 1H), 4.68 (d, J=7.2 Hz, 1H), 4.60 (d, J=9.1 Hz, 2H), 4.48 (t, J=8.4 Hz, 1H), 4.38-4.33 (m, 3H), 4.32-4.28 (m, 1H), 4.15-4.08 (m, 1H), 3.68-3.62 (m, 6H), 3.19-3.15 (m, 5H), 3.13-3.09 (m, 3H), 2.75-2.69 (m, 2H), 2.46 (s, 3H), 2.10-2.06 (m, 2H), 2.00-1.97 (m, 1H), 1.89-1.87 (m, 1H), 1.45-1.42 (m, 3H), 1.40-1.38 (m, 1H), 1.35-1.33 (m, 1H), 1.24 (s, 4H), 1.21-1.18 (m, 3H), 0.98 (s, 9H), 0.86-0.83 (m, 1H).

Example A121. 4-amino-N-((S)-6-(6-(4-((1-(5-chloro-2-(((2S,4R)-1-((S)-2-(1-fluorocyclopropane-1-carboxamido)-3,3-dimethylbutanoyl)-4-hydroxypyrrolidine-2-carboxamido)methyl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

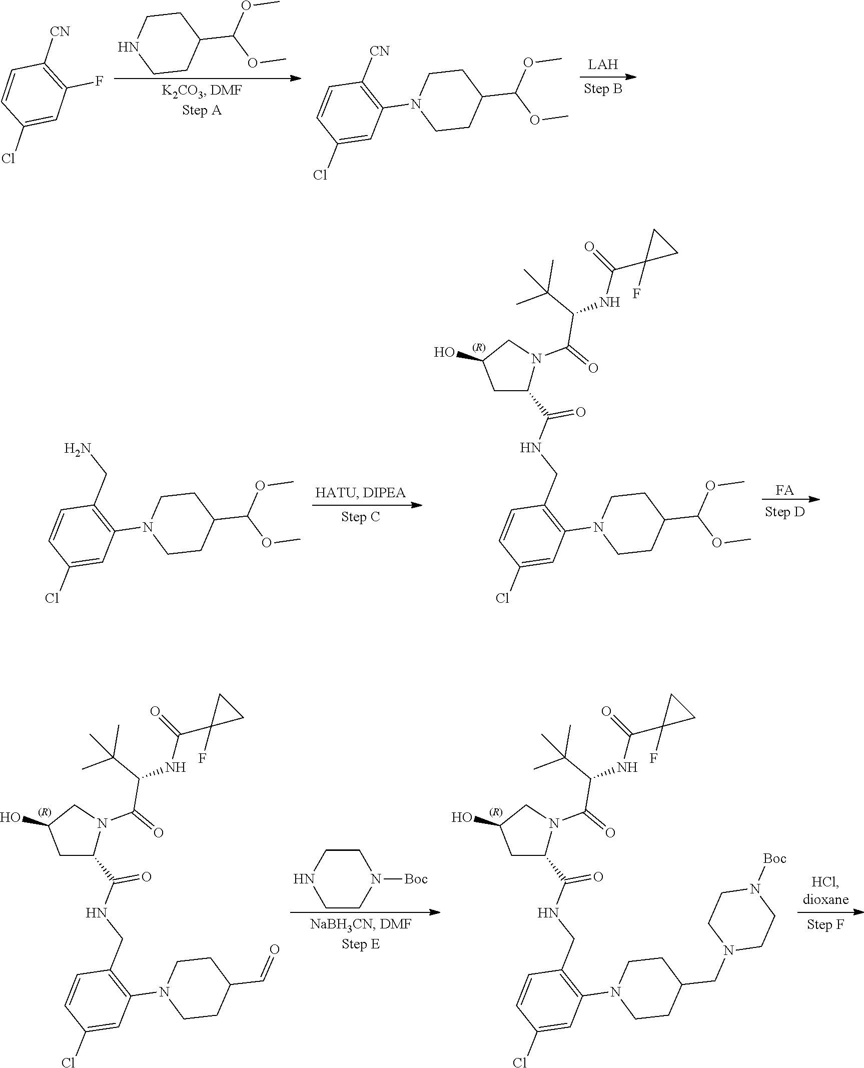

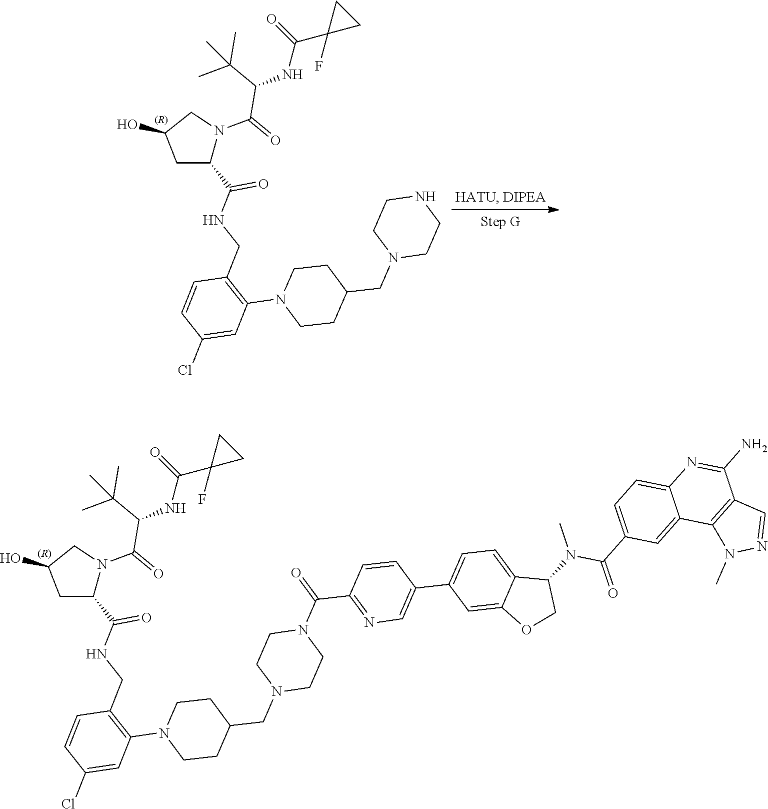

Example A121 was synthesized following the same procedures as described in Example A107 (5.1 mg, 1% yield over 7 steps) as a white solid. LC-MS (ESI): mass calcd. for C₅₉H₆₈ClFN₁₂O₇, 1110.5/z found, 1111.7 [M+H]⁺. ¹HNMR (400 MHz, DMSO-d₆) δ 9.59 (d, J=59.8 Hz, 2H), 8.94 (s, 1H), 8.79 (s, 1H), 8.64 (s, 1H), 8.55-8.37 (m, 2H), 8.28 (d, J=7.8 Hz, 1H), 7.94 (d, J=8.5 Hz, 1H), 7.88 (s, 1H), 7.78 (d, J=8.2 Hz, 1H), 7.61 (s, 1H), 7.46-7.31 (m, 3H), 7.26 (d, J=6.7 Hz, 1H), 7.10-7.01 (m, 2H), 4.76 (s, 1H), 4.60 (d, J=9.2 Hz, 1H), 4.52 (s, 3H), 4.49-4.43 (m, 1H), 4.35 (s, 1H), 4.31-4.23 (m, 2H), 3.68-3.64 (m, 1H), 3.62-3.59 (m, 1H), 3.55-3.47 (m, 4H), 3.34-3.26 (m, 5H), 3.17 (s, 3H), 3.08-3.03 (m, 2H), 2.75-2.63 (m, 5H), 2.10-2.03 (m, 1H), 2.00-1.93 (m, 1H), 1.91-1.80 (m, 3H), 1.47-1.32 (m, 4H), 1.25-1.20 (m, 2H), 0.97 (s, 9H).

### Example A122. 4-amino-N-((S)-6-(6-(4-((1-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

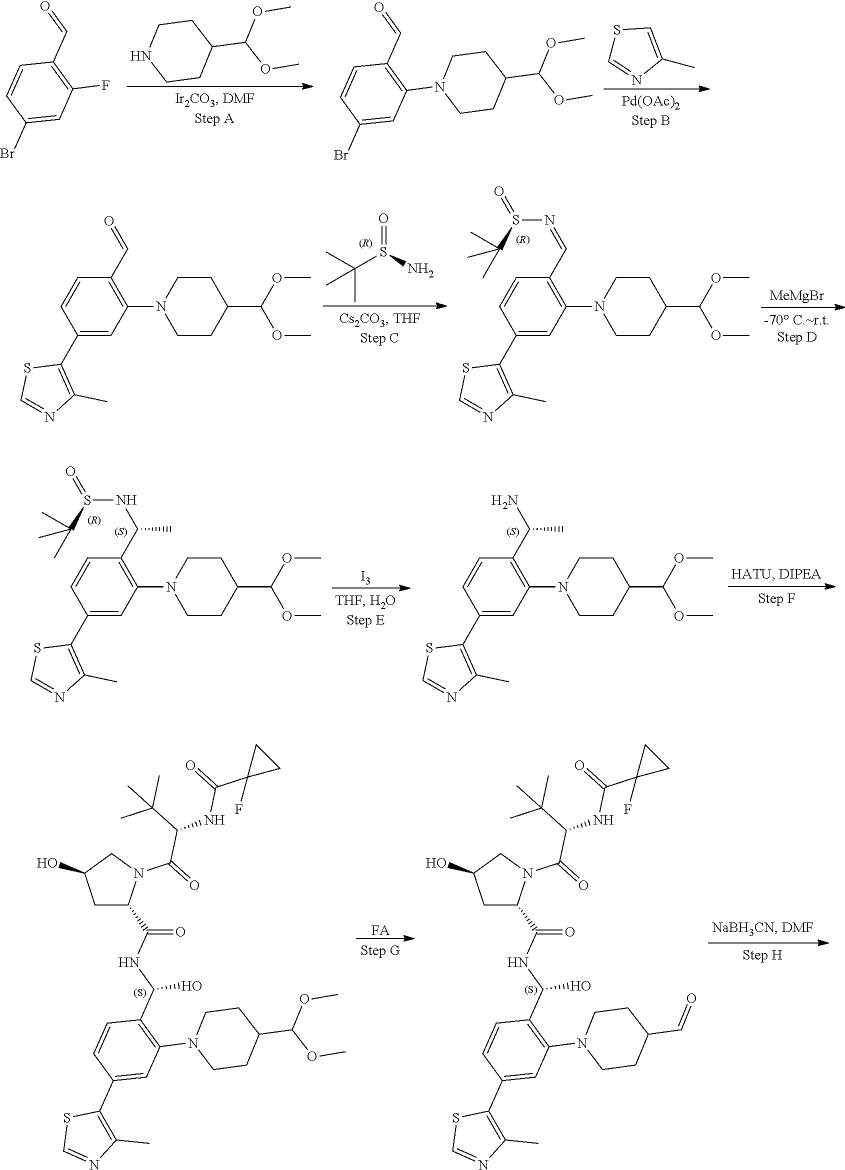

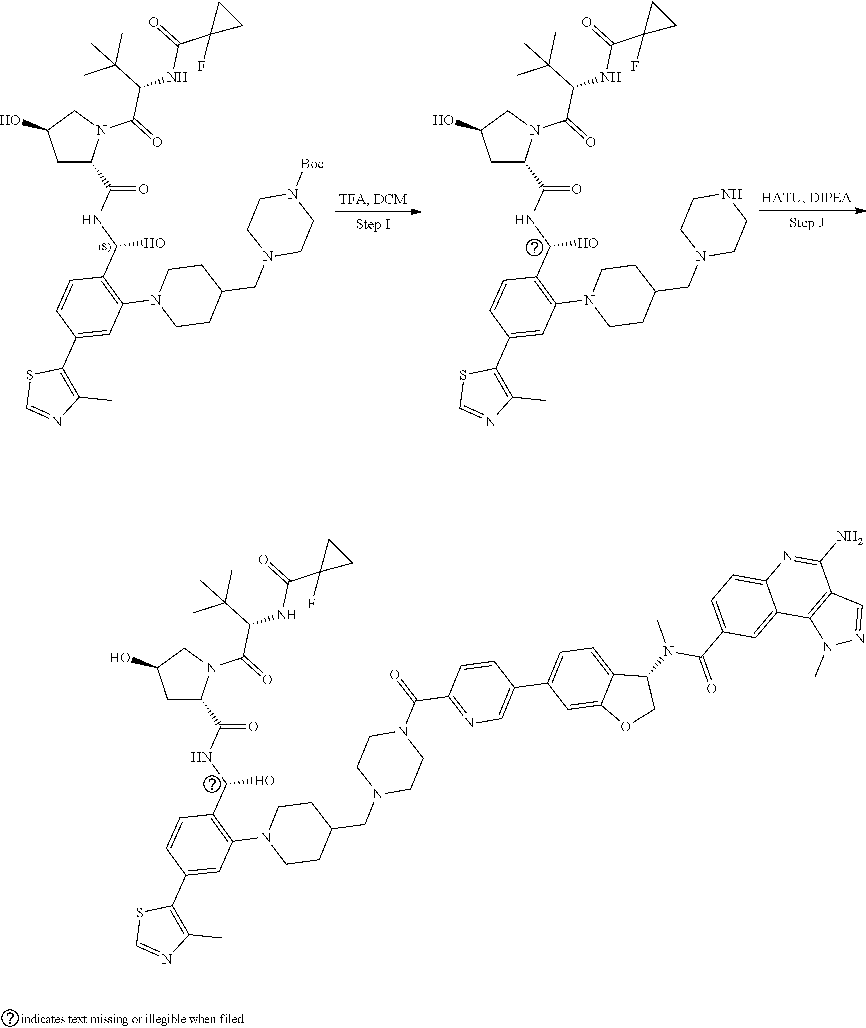

Step C: (R,Z)-N-(2-(4-(dimethoxymethyl)piperidin-1-yl)-4-(4-methylthiazol-5-yl)benzylidene)-2-methylpropane-2-sulfinamide

To a solution of 2-(4-(dimethoxymethyl)piperidin-1-yl)-4-(4-methylthiazol-5-yl)benzaldehyde (1.8 g, 4.99 mmol, 1.0 eq., Example A107) in THF (20 mL) was added (R)-2-methylpropane-2-sulfinamide (1.82 g, 15 mmol, 3.0 eq.) and Cs₂CO₃ (4.07 g, 12.5 mmol, 2.5 eq.) at room temperature. The mixture was then stirred at 60° C. for 16 hours under N₂ atmosphere. The resulting solution was diluted with 30 mL of H₂O and extracted with EtOAc. The combined organic layers were washed with brine, dried over anhydrous Na₂SO₄ and concentrated under reduced pressure. The residue was purified by column chromatography (silica gel, 0% MeOH in DCM~3% MeOH in DCM) to afford (R,Z)-N-(2-(4-(dimethoxymethyl)piperidin-1-yl)-4-(4-methylthiazol-5-yl)benzylidene)-2-methylpropane-2-sulfinamide (1.50 g, 64% yield) as a yellow oil. LC-MS (ESI): mass calcd. For C₂₃H₃₃N₃O₃S₂, 463.2; m/z found, 464.2 [M+H]⁺.

Step D: (R)-N-((S)-1-(2-(4-(dimethoxymethyl)piperidin-1-yl)-4-(4-methylthiazol-5-yl)phenyl)ethyl)-2-methylpropane-2-sulfinamide

To a solution of (R,Z)-N-(2-(4-(dimethoxymethyl)piperidin-1-yl)-4-(4-methylthiazol-5-yl)benzylidene)-2-methylpropane-2-sulfinamide (200 mg, 431 μmol, 1.0 eq.) in DCM (5 mL) was added Magnesium, bromomethyl-(257 mg, 2.16 mmol, 5.0 eq.) at −65° C. The mixture was then stirred at room temperature for 2 hours under N₂ atmosphere. The resulting solution was diluted with 30 mL of NH₄Cl/H₂O and extracted with DCM. The combined organic layers were washed with brine, dried over anhydrous Na₂SO₄ and concentrated under reduced pressure. The residue was purified by column chromatography (silica gel, 0% MeOH in DCM~4% MeOH in DCM) to afford (R)-N-((S)-1-(2-(4-(dimethoxymethyl)piperidin-1-yl)-4-(4-methylthiazol-5-yl)phenyl)ethyl)-2-methylpropane-2-sulfinamide (60 mg, 29% yield) as a yellow oil. LC-MS (ESI): mass calcd. For C₂₄H₃₇N₃O₃S₂, 479.2; m/z found, 480.2 [M+H]⁺.

Steps E-J: 4-amino-N-((S0-6-(6-(4-((1-(2-((S)-1((2S4R)-1-((S)-2-(1-fluorocyclopropane-1-, carboxamido)-3,3-dimethylbutanoyl)-4-hydroxypyrrolidine-2-carboxamido)ethyl)-5-(4-methylthiazol-5-yl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

Example A122 was synthesized following the same procedures as described in Example A107 (4.3 mg, 1% yield over 10 steps) as a white solid. LC-MS (ESI): mass calcd. for C₆₄H₇₄FN₁₃O₇S, 1187.6/z found, 1188.7 [M+H]⁺. ¹HNMR (400 MHz, DMSO-d₆) δ 9.82-9.51 (m, 2H), 9.02-8.98 (m, 1H), 8.97-8.91 (m, 1H), 8.90-8.78 (m, 1H), 8.66-8.62 (m, 1H), 8.46 (s, 1H), 8.36-8.25 (m, 2H), 7.96-7.91 (m, 1H), 7.89-7.84 (m, 1H), 7.82-7.73 (m, 1H), 7.65-7.60 (m, 1H), 7.44-7.36 (m, 2H), 7.30-7.21 (m, 2H), 7.21-7.15 (m, 1H), 5.37-5.29 (m, 1H), 4.84-4.70 (m, 2H), 4.63-4.56 (m, 2H), 4.51 (s, 3H), 4.34-4.19 (m, 4H), 3.63-3.53 (m, 4H), 3.36-3.29 (m, 2H), 3.27-3.23 (m, 2H), 3.17 (s, 3H), 3.05-3.01 (m, 2H), 2.99-2.87 (m, 2H), 2.71 (s, 3H), 2.10-1.85 (m, 4H), 1.83-1.71 (m, 2H), 1.48-1.28 (m, 8H), 1.23 (s, 3H), 0.97 (d, J=6.3 Hz, 9H).

### Example A123. 4-amino-N-((S)-6-(6-(6-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,6-diazaspiro[3.4]octane-2-carbonyl)pyridin-3-yl)-2,3-dihydrobenzofuran-3-yl)-N,1-dimethyl-1H-pyrazolo[4,3-c]quinoline-8-carboxamide

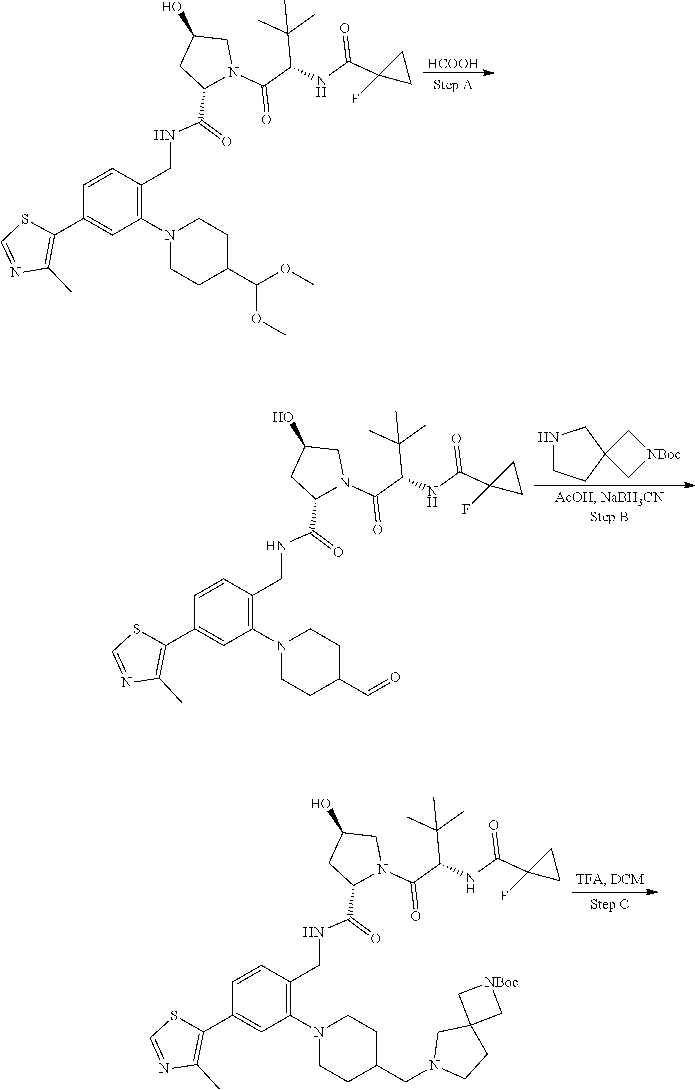

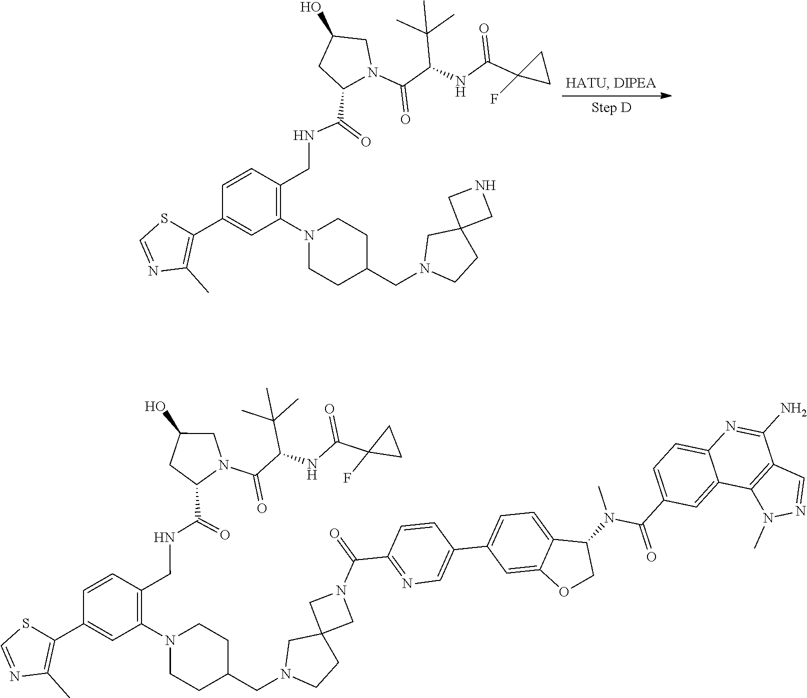 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCC4(CNC4)C3)CC2)c1 ; Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCC4(C3)CN(C(=O)c3ccc(-c5ccc6c(c5)OC[C@H]6N(C)C(=O)c5ccc6nc(N)c7cnn(C)c7c6c5)cn3)C4)CC2)c1]

Example A123 was synthesized following the same procedures as described in Example A107 (1 mg, 2.8% yield over 4 steps) as a white solid. LC-MS (ESI): mass calcd. for C₆₅H₇₄FN₁₃O₇S, 1199.55; m/z found, 1201 [M+H]⁺. ¹H NMR (400 MHz, DMSO-d₆) δ 9.73 (s, 1H), 8.99 (s, 1H), 8.92 (s, 1H), 8.65-8.48 (m, 1H), 8.47-8.39 (m, 1H), 8.30-8.22 (m, 1H), 8.12-8.00 (m, 1H), 8.00-7.64 (m, 2H), 7.64-7.53 (m, 1H), 7.48-7.33 (m, 3H), 7.27 (d, J=8.9 Hz, 2H), 7.17-7.04 (m, 2H), 4.83-4.68 (m, 3H), 4.62-4.59 (m, 1H), 4.56-4.44 (m, 5H), 4.40-4.30 (m, 3H), 4.21-4.16 (m, 1H), 3.67-3.59 (m, 8H), 3.12-3.08 (m, 4H), 2.74-2.69 (m, 5H), 2.46 (s, 3H), 2.13-2.09 (m, 1H), 1.96-1.93 (m, 1H), 1.92-1.83 (m, 3H), 1.69-1.61 (m, 2H), 1.51-1.47 (m, 2H), 1.38-1.35 (m, 2H), 1.22-1.19 (m, 2H), 1.00-0.95 (m, 9H).

### Example A124. 4-amino-N-((S)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl)pyridin-3-yl)-2,3-dihydrobenzofuran-3-yl)-N,1-dimethyl-1H-pyrazolo[4,3-c]quinoline-8-carboxamide

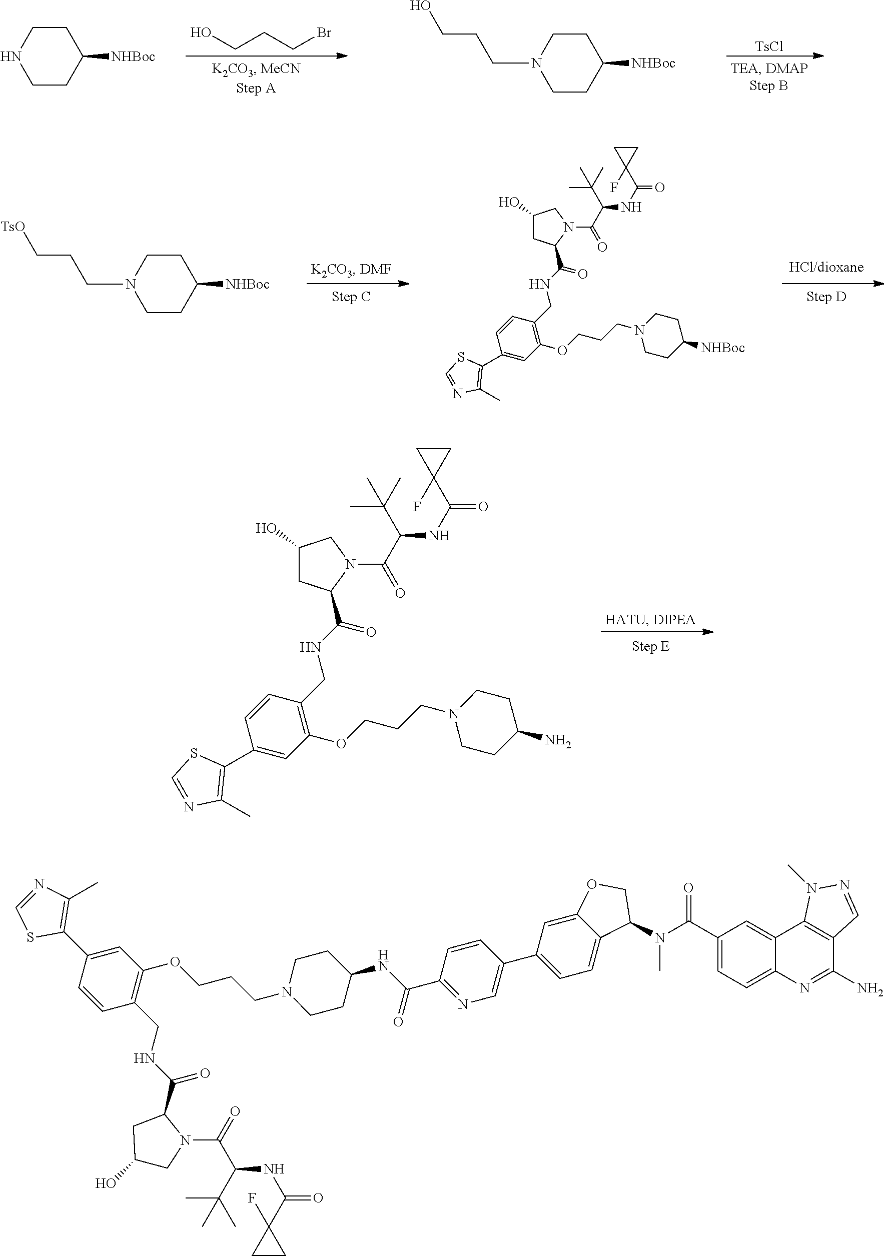

Step A: tert-butyl (1-(3-hydroxypropyl)piperidin-4-yl)carbamate

A mixture of tert-butyl piperidin-4-ylcarbamate (5 g, 25.0 mmol, 1.0 eq.), 3-bromopropan-1-ol (4.2 g, 30.0 mmol, 1.2 eq) and K₂CO₃ (10.3 g, 74.9 mmol, 3.0 eq.) in MeCN (50 mL) was stirred at 80° C. for 14 hours under nitrogen. The resulting solution was diluted with 100 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~5% MeOH in DCM) to afford tert-butyl (1-(3-hydroxypropyl)piperidin-4-yl)carbamate (2 g, 31% yield) as a yellow solid.

LC-MS (ESI): mass calcd, for C₁₃H₂₆N₂O₃, 258.19; m/z found, 259 [M+H]⁺.

Steps B-E: 4-amino-N-(S)-6-16-((1-(3-(2-(2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl)pyridin-3-yl)-2,3-dihydrobenzofuran-3-yl)-N,1-dimethyl-1H-pyrazolo[4,3-c]quinoline-8-carboxamide

Example A124 was synthesized following the same procedures as described in Example A29 (3.6 mg, 7.6% yield over 4 steps) as a white solid. LC-MS (ESI): mass calcd. for C₆₁H₆₉FN₁₂O₈S, 1148.51; m/z found, 1149.7 [M+H]⁺. ¹H NMR (400 MHz, DMSO-d₆) δ 8.99 (s, 1H), 8.94 (s, 1H), 8.63 (d, J=8.4 Hz, 1H), 8.50 (t, J=5.8 Hz, 1H), 8.34 (s, 1H), 8.30-8.26 (m, 2H), 8.14 (s, 1H), 8.08 (d, J=8.3 Hz, 1H), 7.68-7.57 (m, 3H), 7.43-7.38 (m, 2H), 7.35 (s, 1H), 7.31-7.26 (m, 1H), 7.18 (s, 2H), 7.01 (s, 1H), 6.97 (d, J=7.8 Hz, 1H), 5.17 (s, 1H), 4.77 (s, 2H), 4.62-4.58 (m, 1H), 4.54-4.49 (m, 1H), 4.43 (s, 3H), 4.36 (s, 1H), 4.31-4.23 (m, 2H), 4.11 (s, 2H), 3.95-3.80 (m, 2H), 3.64-3.62 (m, 1H), 3.06-3.02 (m, 2H), 2.72 (s, 3H), 2.69-2.64 (m, 2H), 2.47 (s, 3H), 2.34-2.25 (m, 2H), 2.11-2.07 (m, 1H), 2.01-1.92 (m, 3H), 1.84-1.71 (m, 4H), 1.40-1.33 (m, 2H), 1.24-1.20 (m, 2H), 0.96 (s, 9H).

### Example A125. 4-amino-N-((S)-6-(6-(((R)-1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl) pyrrolidin-3-yl)carbamoyl)pyridin-3-yl)-2,3-dihydrobenzofuran-3-yl)-N,1-dimethyl-1H-pyrazolo[4,3-c]quinoline-8-carboxamide

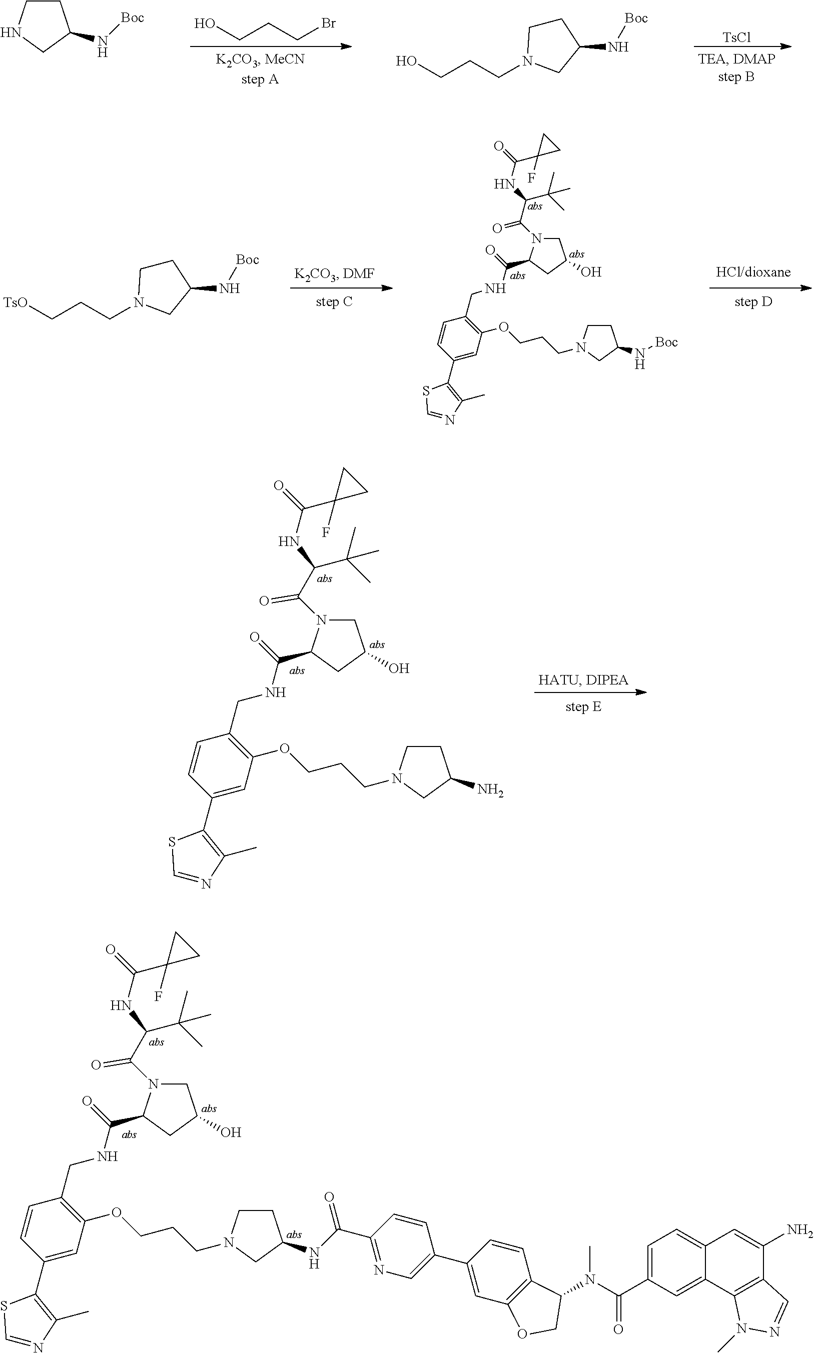

Example A125 was synthesized following the same procedures as described in Example A29 (5.0 mg, 0.2% yield over 5 steps) as a white solid. LC-MS (ESI): mass calcd. for C₆₀H₆₇FN₁₂O₈S, 1134.5/z found, 1135.5 [M+H]⁺. ¹HNMR (400 MHz, DMSO-d₆) δ 8.97 (s, 1H), 8.95-8.69 (m, 2H), 8.50 (t, J=5.8 Hz, 1H), 8.34 (s, 1H), 8.29 (d, J=10.1 Hz, 1H), 8.27 (s, 1H), 8.14 (s, 1H), 8.09 (d, J=8.2 Hz, 1H), 7.68-7.63 (m, 2H), 7.61-7.55 (m, 1H), 7.43-7.38 (m, 2H), 7.35 (s, 1H), 7.28 (d, J=7.0 Hz, 1H), 7.22-7.15 (m, 2H), 7.02 (d, J=6.9 Hz, 1H), 6.97 (d, J=7.7 Hz, 1H), 5.24-5.11 (m, 1H), 4.77 (s, 2H), 4.59 (d, J=9.2 Hz, 1H), 4.53 (d, J=8.4 Hz, 1H), 4.43 (s, 3H), 4.35 (s, 1H), 4.33-4.23 (m, 2H), 4.13 (t, J=5.7 Hz, 2H), 3.66-3.58 (m, 2H), 3.50-3.43 (m, 2H), 3.06-2.83 (m, 5H), 2.72 (s, 3H), 2.45 (s, 3H), 2.31-2.25 (m, 1H), 2.10-2.00 (m, 3H), 1.96-1.88 (m, 2H), 1.39-1.32 (m, 2H), 1.24-1.20 (m, 2H), 0.95 (s, 9H).

### Example A126. 2-amino-N-((S)-6-(6-((4-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperazin-1-yl)butyl)carbamoyl)pyridin-3-yl)-2,3-dihydrobenzofuran-3-yl)-N,3-dimethylquinoline-6-carboxamide

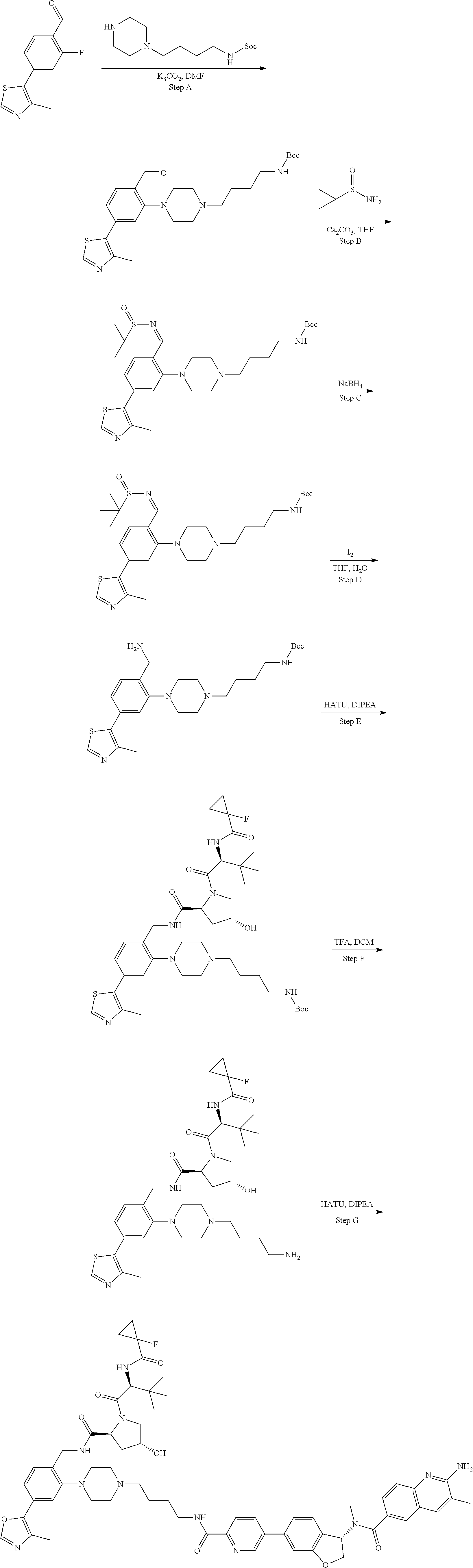

Step A: tert-butyl (4-(4-(2-formyl-5-(4-methylthiazol-5-yl)phenyl)piperazin-1-yl)butyl)carbamate

To a solution of 2-fluoro-4-(4-methylthiazol-5-yl)benzaldehyde (500 mg, 2.26 mmol, 1.0 eq.) and tert-butyl (4-(piperazin-1-yl)butyl)carbamate (580 mg, 2.26 mmol, 1.0 eq.) in DMF (10 mL) was added K₂CO₃ (634 mg, 4.52 mmol, 2.0 eq.) The resulting mixture was stirred at 80° C. for 16 hours. The reaction mixture was quenched with H₂O and then extracted with EtOAc (50 mL×3). The combined organic layers were washed with brine, dried over anhydrous Na₂SO₄ and concentrated. The residue was purified by column chromatography (silica gel, 0% EtOAc in PE~100% EtOAc in PE) to afford tert-butyl (4-(4-(2-formyl-5-(4-methylthiazol-5-yl)phenyl)piperazin-1-yl)butyl)carbamate (500 mg, 55% yield) as a white solid. LC-MS (ESI): mass calcd. For C₂₄H₃₄N₄O₃S, 468.24; m/z found, 469.24 [M+H]⁺.

Step B: tert-butyl (Z)-(4-(4-(2-(((tert-butylsulfinyl)imino)methyl)-5-(4-methylthiazol-5-yl)phenyl)piperazin-1-yl)butyl)carbamate

To a stirred mixture of tert-butyl (4-(4-(2-formyl-5-(4-methylthiazol-5-yl)phenyl)piperazin-1-yl)butyl)carbamate (500 mg, 1.01 mol, 1.0 eq.) in THF (10 mL) were added 2-methylpropane-2-sulfinamide (125 mg, 1.01 mmol, 1.0 eq.) and Cs₂CO₃ (662 mg, 2.02 mmol, 2.0 eq.) at room temperature. The resulting mixture was stirred at 70° C. for 15 hours. The reaction mixture was poured into water (50 mL), and then extracted with EtOAc (50 mL×3). The combined organic layers were washed with brine, dried over anhydrous Na₂SO₄ and concentrated. The residue was purified by column chromatography (silica gel, 0% EtOAc in PE~100% EtOAc in PE) to afford methyl ert-butyl (Z)-(4-(4-(2-(((tert-butylsulfinyl)imino)methyl)-5-(4-methylthiazol-5-yl)phenyl)piperazin-1-yl)butyl)carbamate (400 mg, 80% yield) as a white solid.

LC-MS (ESI): mass calcd. For C₂₈H₄₃N₅O₃S₂, 561.2; m/z found, 562.2 [M+H]⁺.

Step C: tert-butyl (4-(4-(2-(((tert-butylsulfinyl)amino)methyl)-5-(4-methylthiazol-5-yl)phenyl)piperazin-1-yl)butyl)carbamate

To a solution of methyl ert-butyl (Z)-(4-(4-(2-(((tert-butylsulfinyl)imino)methyl)-5-(4-methylthiazol-5-yl)phenyl)piperazin-1-yl)butyl)carbamate (400 mg, 710 μmol, 1.0 eq.) in MeOH (5 mL) was added NaBH₄ (47 mg, 1.42 mmol, 2.0 eq.). The reaction mixture was stirred at room temperature for 1 hour. The reaction mixture was poured into water (50 mL), and then extracted with EtOAc (50 mL×3). The combined organic layers were washed with brine, dried over anhydrous Na₂SO₄ and concentrated. The residue was purified by column chromatography (silica gel, 0% MeOH in DCM~10% MeOH in DCM) to afford tert-butyl (4-(4-(2-(((tert-butylsulfinyl)amino)methyl)-5-(4-methylthiazol-5-yl)phenyl)piperazin-1-yl)butyl)carbamate (350 mg, 85% yield) as a white solid. LC-MS (ESI): mass calcd. For C₂₈H₄₅N₅O₃S₂, 563.3; m/z found, 564.3 [M+H]⁺.

Step D: tert-butyl (4-(4-(2-(aminomethyl)-5-(4-methylthiazol-5-yl)phenyl)piperazin-1-yl)butyl)carbamate

To a stirred mixture of tert-butyl (4-(4-(2-(((tert-butylsulfinyl)amino)methyl)-5-(4-methylthiazol-5-yl)phenyl)piperazin-1-yl)butyl)carbamate (350 mg, 620 μmol, 1.0 eq.) in THF (4 mL) and H₂O (1 mL) was added 12 (315 mg, 1.24 mmol, 2.0 eq.) The resulting mixture was stirred at 50° C. for 1 hour. The reaction mixture was poured into water (30 mL) and then extracted with EtOAc (30 mL×3). The combined organic layers were washed with brine, dried over anhydrous Na₂SO₄ and concentrated to afford ert-butyl (4-(4-(2-(aminomethyl)-5-(4-methylthiazol-5-yl)phenyl)piperazin-1-yl)butyl)carbamate (300 mg, crude) as a white solid. LC-MS (ESI): mass calcd. For C₂₄H₃₇N502S, 459.3; m/z found, 460.3 [M+H]⁺.

Steps E-G: 2-amino-N-(S)-6-16-(4-(4-(2-(2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperazin-1-yl)butyl)carbamoyl)pyridin-3-yl)-2,3-dihydrobenzofuran-3-yl)-N,3-dimethylquinoline-6-carboxamide

Example A126 was synthesized following the same procedures as described in Example A23 (3.8 mg, 5% yield over 3 steps) as a white solid. LC-MS (ESI): mass calcd. for C₆₀H₇₀FN₁₁O₇S, 1107.52; m/z found, 1108.5 [M+H]⁺, ¹HNMR (400 MHz, DMSO-d₆) δ 9.08-8.90 (m, 2H), 8.87 (s, 1H), 8.50 (s, 1H), 8.27 (d, J=8.0 Hz, 1H), 8.14 (s, 1H), 8.09 (d, J=7.8 Hz, 1H), 7.80 (d, J=13.0 Hz, 2H), 7.53 (d, J=14.3 Hz, 2H), 7.44 (d, J=8.0 Hz, 1H), 7.39 (d, J=7.2 Hz, 1H), 7.34 (s, 1H), 7.27 (d, J=8.8 Hz, 1H), 7.12 (s, 2H), 6.53 (s, 2H), 5.16 (s, 1H), 4.82-4.72 (m, 1H), 4.70-4.66 (m, 1H), 4.61-4.56 (m, 1H), 4.49-4.39 (m, 2H), 4.38-4.22 (m, 3H), 3.67-3.57 (m, 6H), 2.94 (d, J=14.7 Hz, 4H), 2.73-2.66 (m, 5H), 2.58-2.53 (m, 2H), 2.45 (s, 3H), 2.22 (s, 3H), 2.15-1.96 (m, 2H), 1.95-1.75 (m, 2H), 1.59-1.54 (m, 2H), 1.38-1.31 (m, 2H), 1.25-1.21 (m, 2H), 0.97 (s, 9H)

### Example A127. N-(cyclopropylmethyl)-N-((S)-6-(4-(((18,4S)-4-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)phenyl)-2,3-dihydrobenzofuran-3-yl)-3-methylquinoline-6-carboxamide

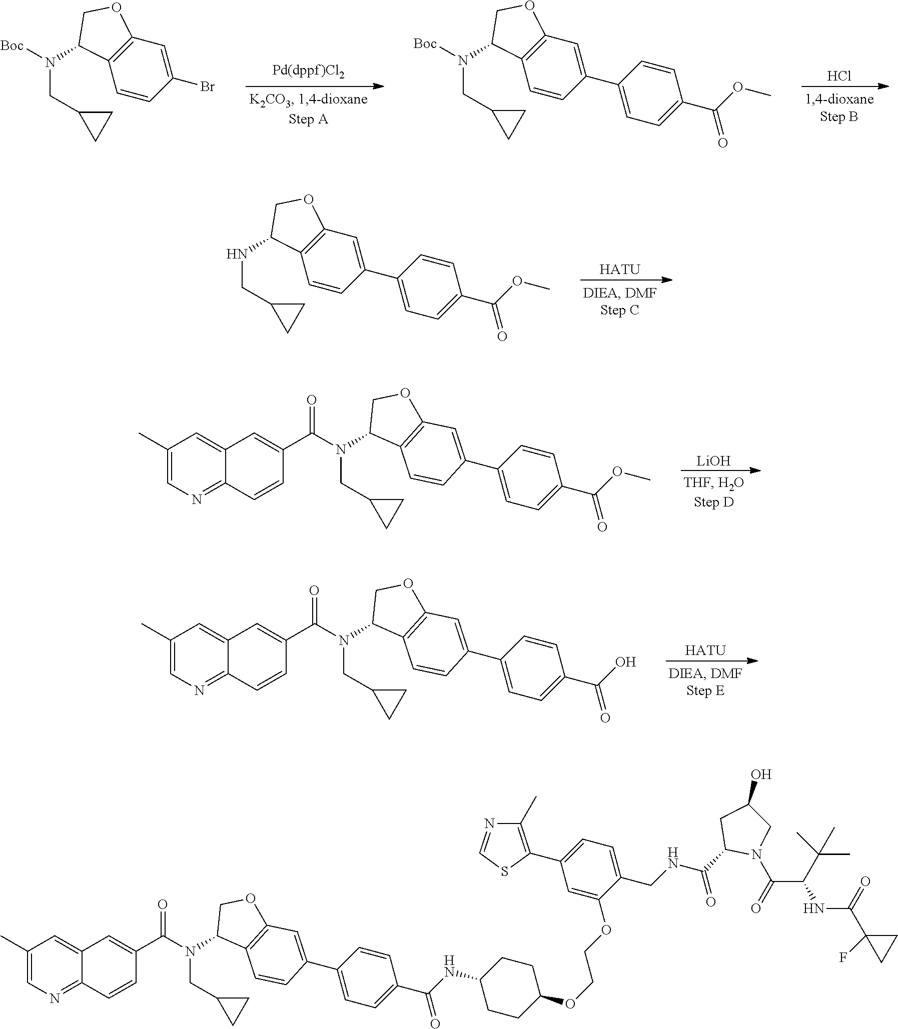

Example A127 was synthesized following the same procedures as described in Example A51 (3.2 mg, 6% yield over 5 steps) as a white solid. LC-MS (ESI): mass calcd. for C₆₄H₇₂FNO₉S, 1134.38; m/z found, 1134 [M+H]⁺. ¹H NMR (400 MHz, DMSO-d₆) δ 8.99 (s, 1H), 8.84 (d, J=1.8 Hz, 1H), 8.50 (t, J=5.8 Hz, 1H), 8.33-8.17 (m, 2H), 8.11-8.00 (m, 2H), 7.95-7.88 (m, 2H), 7.80-7.70 (m, 3H), 7.60-7.53 (m, 1H), 7.41 (d, J=7.8 Hz, 1H), 7.36-7.26 (m, 2H), 7.22 (s, 1H), 7.05 (s, 1H), 6.97 (d, J=7.6 Hz, 1H), 5.17 (s, 1H), 4.94-4.64 (m, 2H), 4.60 (d, J=9.3 Hz, 1H), 4.52 (t, J=8.3 Hz, 1H), 4.36 (s, 1H), 4.30 (d, J=5.9 Hz, 1H), 4.24 (d, J=5.5 Hz, 1H), 4.20-4.14 (m, 2H), 4.07 (q, J=7.1 Hz, 1H), 3.83-3.79 (m, 2H), 3.75-3.70 (m, 1H), 3.66-3.58 (m, 2H), 3.21-3.11 (m, 2H), 3.10-3.05 (m, 1H), 2.49 (s, 3H), 2.47 (s, 3H), 2.13-2.04 (m, 3H), 1.96-1.85 (m, 3H), 1.41-1.27 (m, 6H), 1.23-1.21 (m, 4H), 0.96 (s, 9H), 0.40-0.23 (m, 2H), −0.19-0.31 (m, 1H).

### Example A128. 4-amino-N-((S)-6-(6-((7-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) heptyl)carbamoyl)pyridin-3-yl)-2,3-dihydrobenzofuran-3-yl)-N,1-dimethyl-1H-pyrazolo[4,3-c]quinoline-8-carboxamide

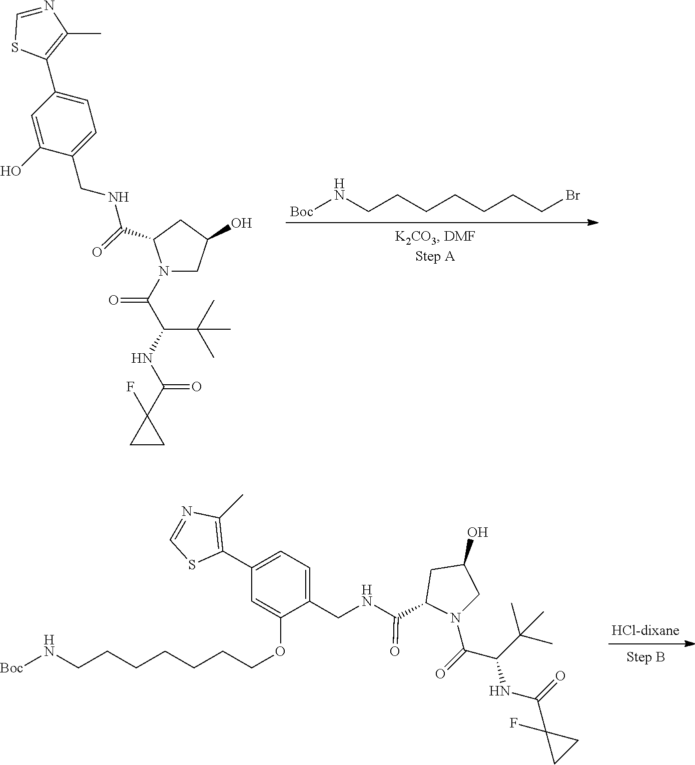 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)NCCCCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCCCCNC(=O)OC(C)(C)C)c1]

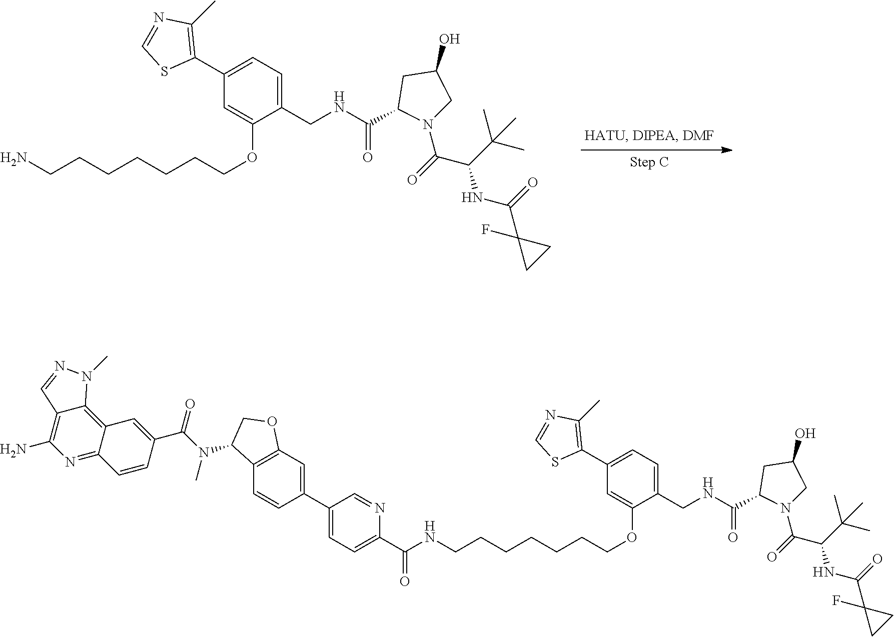 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCCNC(=O)c2ccc(-c3ccc4c(c3)OC[C@H]4N(C)C(=O)c3ccc4nc(N)c5cnn(C)c5c4c3)cn2)c1]

Example A128 was synthesized following the same procedures as described in Example A9 (2.0 mg, 10% yield over 3 steps) as a white solid. LC-MS (ESI): mass calcd. for C₆₀H₆₈FN₁₁O₈S, 1121.5; m/z found, 1122.7 [M+H]⁺. ¹HNMR (400 MHz, DMSO-d₆) δ 8.97 (s, 1H), 8.92 (s, 1H), 8.79 (t, J=6.1 Hz, 1H), 8.55 (s, 1H), 8.49 (t, J=6.0 Hz, 1H), 8.41 (s, 1H), 8.26 (d, J=8.2 Hz, 1H), 8.07 (d, J=8.2 Hz, 1H), 7.88-7.77 (m, 2H), 7.59 (s, 1H), 7.43-7.38 (m, 2H), 7.35 (s, 1H), 7.28 (d, J=6.8 Hz, 1H), 7.16 (s, 1H), 7.06-6.97 (m, 2H), 6.94 (d, J=7.7 Hz, 1H), 5.17 (d, J=3.3 Hz, 1H), 4.86-4.65 (m, 2H), 4.59 (d, J=8.8 Hz, 1H), 4.54-4.46 (m, 4H), 4.34 (s, 1H), 4.32-4.17 (m, 2H), 4.04 (t, J=6.3 Hz, 2H), 3.71-3.47 (m, 2H), 3.31-3.23 (m, 3H), 2.71 (s, 3H), 2.45 (s, 3H), 2.13-1.97 (m, 2H), 1.96-1.88 (m, 1H), 1.80-1.72 (m, 2H), 1.60-1.54 (m, 2H), 1.50-1.44 (m, 2H), 1.39-1.32 (m, 5H), 1.24-1.23 (m, 2H), 0.95 (s, 9H).

### Example A129. N-(cyclopropylmethyl)-N-((S)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl)pyridin-3-yl)-2,3-dihydrobenzofuran-3-yl)-3-methylquinoline-6-carboxamide

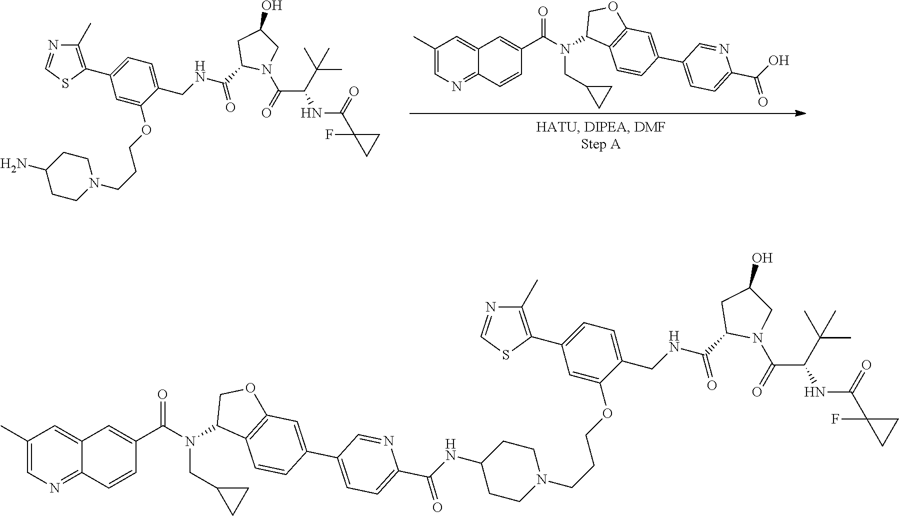

A mixture of (2S,4R)-N-(2-(3-(4-aminopiperidin-1-yl) propoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20 mg, 29.7 μmol, 1.0 eq.), (S)-5-(3-(N-(cyclopropylmethyl)-3-methylquinoline-6-carboxamido)-2,3-dihydrobenzofuran-6-yl)picolinic acid (16 mg, 32.7 μmol, 1.1 eq.), HATU (23 mg, 59.4 μmol, 2 eq.) and DIPEA (12 mg, 89.2 μmol, 3.0 eq.) in DMF (3 mL) was stirred at room temperature for 30 mins under nitrogen. The resulting solution was diluted with 50 mL of water and extracted with EtOAc (30 mL×2). The combined organic layers were washed with brine (50 mL), dried over Na₂SO₄ and concentrated under reduced pressure. The crude product was purified by Prep-HPLC (C18, 20~95%, MeCN in H₂O with 0.1% HCOOH) to afford N-(cyclopropylmethyl)-N-((S)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl)pyridin-3-yl)-2,3-dihydrobenzofuran-3-yl)-3-methylquinoline-6-carboxamide (9.7 mg, 29% yield) as a white solid.

LC-MS (ESI): mass calcd, for C₆₃H₇₂FN₉O₈S, 1133.52; m/z found, 1134.8 [M+H]⁺. ¹H NMR (400 MHz, DMSO-d₆) δ 8.99 (s, 1H), 8.93 (s, 1H), 8.84 (d, J=2.1 Hz, 1H), 8.63-8.58 (m, 1H), 8.49 (d, J=6.0 Hz, 1H), 8.30-8.22 (m, 2H), 8.14 (s, 1H), 8.09-8.04 (m, 3H), 7.79-7.73 (m, 1H), 7.64-7.61 (m, 1H), 7.42-7.37 (m, 2H), 7.32-7.27 (m, 2H), 7.01 (s, 1H), 6.98-6.95 (m, 1H), 5.17 (s, 1H), 4.99-4.64 (m, 2H), 4.61-4.57 (m, 1H), 4.55-4.49 (m, 1H), 4.36 (s, 1H), 4.31-4.21 (m, 2H), 4.13-4.08 (m, 2H), 3.92-3.80 (m, 1H), 3.68-3.58 (m, 2H), 3.20-3.13 (m, 2H), 3.11-2.95 (m, 4H), 2.63-2.59 (m, 1H), 2.49 (s, 3H), 2.47 (s, 3H), 2.30-2.17 (m, 2H), 2.10-2.06 (m, 1H), 1.99-1.92 (m, 3H), 1.85-1.79 (m, 2H), 1.75-1.66 (m, 2H), 1.41-1.33 (m, 2H), 1.24-1.20 (m, 2H), 0.97-0.91 (m, 11H), 0.38-0.26 (m, 2H), −0.18-0.25 (m, 1H).

### Example A130. 4-amino-N-((S)-6-(6-(((1S,4S)-4-(ethyl(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino)cyclohexyl) carbamoyl)pyridin-3-yl)-2,3-dihydrobenzofuran-3-yl)-N,1-dimethyl-1H-pyrazolo[4,3-c]quinoline-8-carboxamide

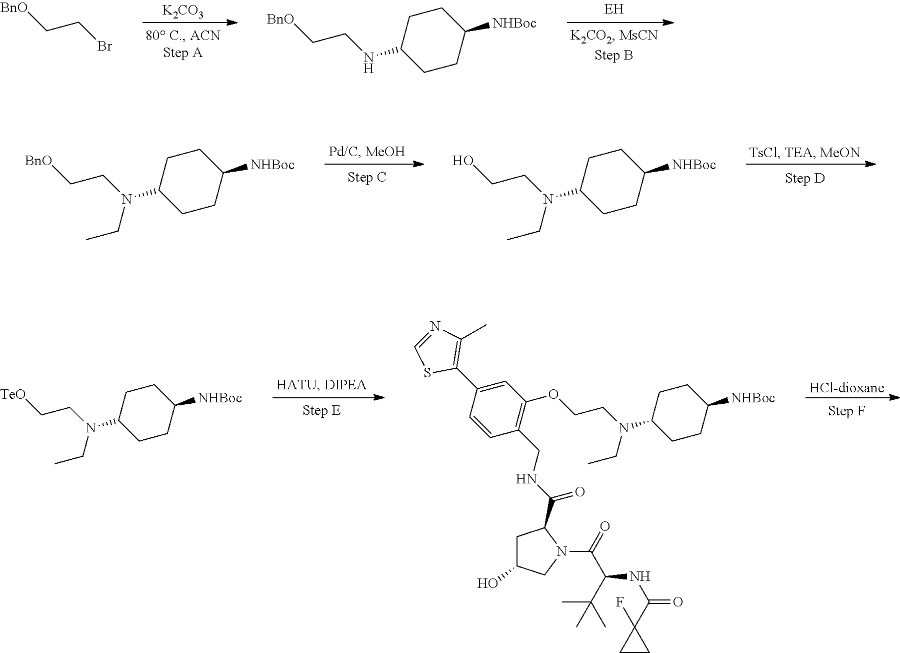

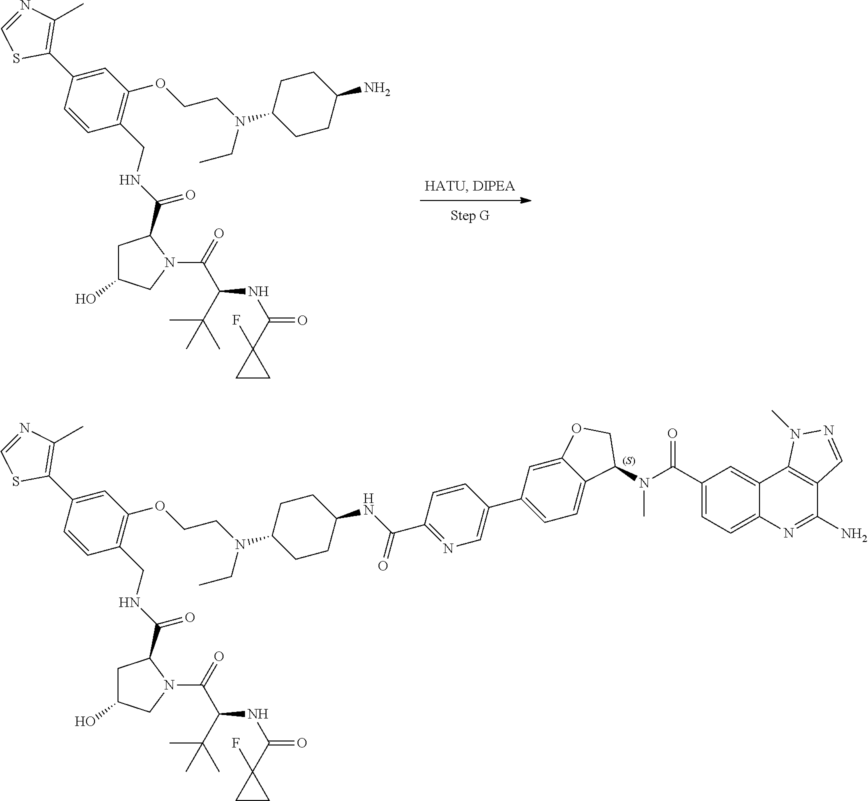

Step A: tert-butyl ((1r,4r)-4-((2-(benzyloxy)ethyl)amino)cyclohexyl)carbamate

To a solution of tert-butyl ((1r,4r)-4-aminocyclohexyl)carbamate (3.00 g, 14.0 mmol, 1.0 eq.), ((2-bromoethoxy)methyl) benzene (2.4 g, 11.2 mmol, 0.8 eq.) and K₂CO₃ (3.87 g, 28.0 mmol, 2.0 eq.) in MeCN (60 mL). The mixture was stirred at 80° C. for 16 hours. The resulting solution was diluted with 50 mL of water and extracted with EtOAc (90 mL×2). The combined organic layers were washed with brine (50 mL), dried over Na₂SO₄ and concentrated under reduced pressure. The residue was purified by flash column chromatography on silica gel (100-200 mesh silica gel, 0-2% MeOH in DCM) to give tert-butyl ((1r,4r)-4-((2-(benzyloxy)ethyl)amino)cyclohexyl)carbamate (1.6 g, 32% yield) as a yellow solid. LC-MS (ESI): mass calcd. For C₂₀H₃₂N₂O₃, 348.24; m/z found, 349 [M+H]⁺.

Step B: tert-butyl ((1r,4r)-4-((2-(benzyloxy)ethyl)(ethyl)amino)cyclohexyl)carbamate

A mixture of tert-butyl ((1r,4r)-4-((2-(benzyloxy)ethyl)amino)cyclohexyl)carbamate (250 mg, 717 μmol 1.0 eq.), tert-butyl ((1r,4r)-4-((2-(benzyloxy)ethyl)amino)cyclohexyl)carbamate (250 mg, 717 μmol, 1.0 eq.) and K₂CO₃ (297 mg, 2.15 mmol, 3.0 eq.) in MeCN (10 mL) was stirred at 55° C. for 16 hours under nitrogen. After cooling to room temperature, The resulting solution was diluted with 20 mL of water and extracted with EtOAc (30 mL×3). The combined organic layers were washed with brine (50 mL), dried over Na₂SO₄ and concentrated under reduced pressure. The crude product was purified by flash column chromatography on silica gel (100-200 mesh silica gel, 0~5% MeOH in DCM) to afford ter-butyl (1r,4r)-4-(2-(benzyloxy)ethyl)(ethyl)amino)cyclohexyl)carbamate (150 mg, 55.5% yield) as a white solid.

LC-MS (ESI): mass calcd. For C₂₂H₃₆N₂O₃, 376.27; m/z found, 377 [M+H]⁺.

Step C: tert-butyl ((1r,4r)-4-(ethyl(2-hydroxyethyl)amino)cyclohexyl)carbamate

A mixture of tert-butyl ((1r,4r)-4-((2-(benzyloxy)ethyl)(ethyl)amino)cyclohexyl)carbamate (150 mg, 619 μmol, 1.0 eq.), Pd/C (150 mg, 10% Wt) and Pd (OH)/C (150 mg, 10% Wt) in MeOH (10 mL) was stirred at 35° C. for 16 hours under nitrogen. The solution was filtered by pad celite to give tert-butyl ((1r,4r)-4-(ethyl(2-hydroxyethyl)amino)cyclohexyl)carbamate (30 mg, 17% yield) as a white solid. LC-MS (ESI): mass calcd. For C₁₅H₃₀N₂O₃, 286.23; m/z found, 287 [M+H]⁺.

Steps D-G: 4-amino-N-(S)-6-(6-((1S,4S)-4-(ethyl(2-(2-(((2S,4R)-1-(S)-2-(1-fluorocyclopropane-1-carboxamido)-3, 3-dimethylbutanoyl)-4-hydroxypyrrolidine-2-carboxamido)methyl)-5-(4-methylthiazol-5-yl)phenoxy)ethyl)amino)cyclohexyl) carbamoyl)pyridin-3-yl)-2,3-dihydrobenzofuran-3-yl)-N,1-dimethyl-1H-pyrazolo[4,3-c]quinoline-8-carboxamide

Example A130 was synthesized following the same procedures as described in Example A119 (2.4 mg, 8% yield over 4 steps) as a white solid. LC-MS (ESI): mass calcd. for C₆₄H₆₉FN₈O₉S, 1144.49; m/z found, 1145.5 [M+H]⁺. ¹HNMR (400 MHz, DMSO-d₆) δ 8.99 (s, 1H), 8.92 (s, 1H), 8.51-8.43 (m, 2H), 8.34 (s, 1H), 8.29-8.25 (m, 2H), 8.15 (s, 2H), 8.08 (d, J=8.2 Hz, 1H), 7.68-7.58 (m, 3H), 7.42-7.37 (m, 2H), 7.34 (s, 1H), 7.28 (d, J=6.5 Hz, 1H), 7.19-7.15 (m, 2H), 7.03 (s, 1H), 6.98-6.94 (m, 1H), 5.17 (s, 1H), 4.81-4.72 (m, 2H), 4.59 (d, J=9.0 Hz, 1H), 4.55-4.50 (m, 1H), 4.43 (s, 3H), 4.36-4.34 (m, 1H), 4.31-4.19 (m, 2H), 4.08-4.04 (m, 2H), 3.83-3.73 (m, 2H), 3.64-3.61 (m, 1H), 3.46-3.45 (m, 1H), 3.44-3.43 (m, 1H), 2.95-2.90 (m, 2H), 2.72 (s, 3H), 2.68-2.65 (m, 2H), 2.47 (s, 3H), 2.12-2.07 (m, 1H), 1.97-1.93 (m, 1H), 1.92-1.87 (m, 2H), 1.85-1.79 (m, 2H), 1.52-1.41 (m, 4H), 1.39-1.34 (m, 2H), 1.23-1.19 (m, 2H), 1.03 (t, J=6.9 Hz, 3H), 0.95 (s, 9H).

### Example A131. 2-amino-N-((S)-6-(6-((4-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperazin-1-yl)butyl)carbamoyl)pyridin-3-yl)-2,3-dihydrobenzofuran-3-yl)-N-isopropyl-3-methylquinoline-6-carboxamide

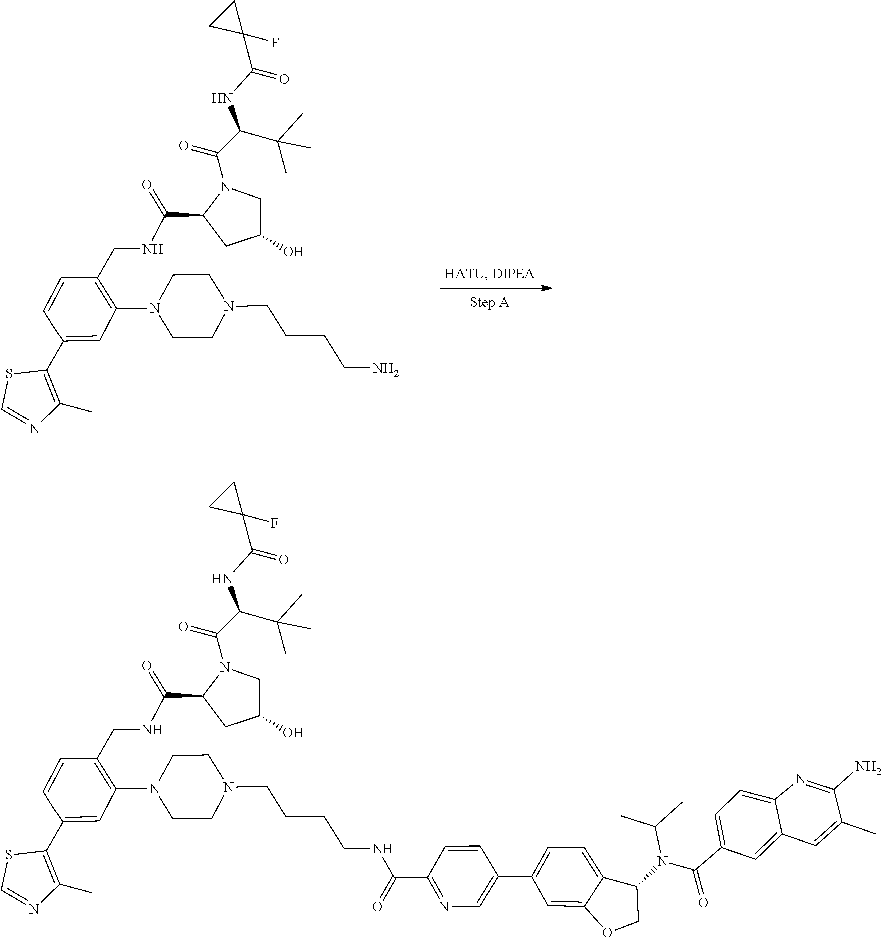

Example A131 was synthesized following the same procedures as described in Example A23 (2.3 mg, 8% yield). LC-MS (ESI): mass calcd, for C₆₂H₇₄FN₁₁O₇S, 1135.55/z found, 1136.55 [M+H]⁺. ¹HNMR (400 MHz, DMSO-d₆) δ 8.97 (s, 1H), 8.90 (s, 1H), 8.85-8.81 (m, 1H), 8.49 (t, J=5.6 Hz, 1H), 8.23 (d, J=8.2 Hz, 1H), 8.20 (s, 1H), 8.07 (d, J=8.1 Hz, 1H), 7.78 (s, 1H), 7.61 (s, 1H), 7.48-7.36 (m, 4H), 7.29-7.25 (m, 2H), 7.22-7.16 (m, 1H), 7.12-7.09 (m, 2H), 6.47-6.43 (m, 2H), 4.77-4.66 (m, 2H), 4.59 (d, J=9.2 Hz, 1H), 4.49-4.44 (m, 1H), 4.41-4.24 (m, 3H), 3.68-3.63 (m, 1H), 3.62-3.58 (m, 1H), 3.51-3.37 (m, 6H), 2.95-2.84 (m, 5H), 2.59-2.52 (m, 2H), 2.45 (s, 3H), 2.40-2.36 (m, 2H), 2.21 (s, 3H), 2.10-2.05 (m, 1H), 1.94-1.88 (m, 1H), 1.62-1.56 (m, 2H), 1.52-1.48 (m, 2H), 1.41-1.37 (m, 3H), 1.35-1.32 (m, 1H), 1.27-1.12 (m, 6H), 0.97 (s, 9H).

### Example A132. 4-amino-N-((S)-6-(6-((6-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) hexyl)carbamoyl)pyridin-3-yl)-2,3-dihydrobenzofuran-3-yl)-N,1-dimethyl-1H-pyrazolo[4,3-c]quinoline-8-carboxamide

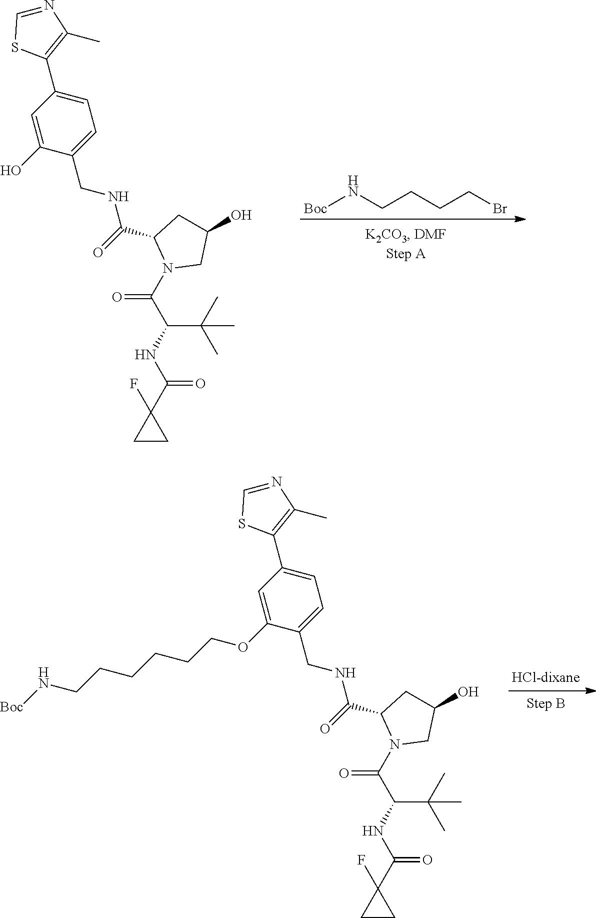 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)NCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCCCNC(=O)OC(C)(C)C)c1]

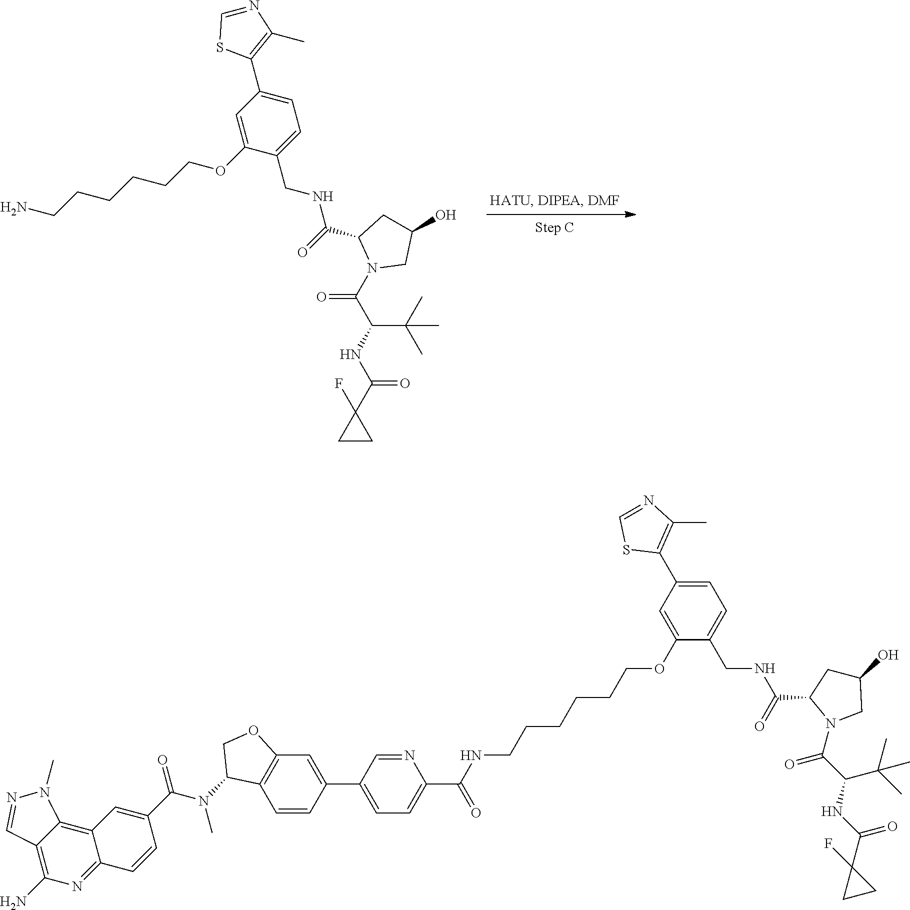 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCCNC(=O)c2ccc(-c3ccc4c(c3)OC[C@H]4N(C)C(=O)c3ccc4nc(N)c5cnn(C)c5c4c3)cn2)c1]

Example A132 was synthesized following the same procedures as described in Example A23 (4.0 mg, 10% yield over 3 steps) as a white solid. LC-MS (ESI): mass calcd. for C₅₉H₆₆FN₁₁O₈S, 1107.48; m/z found, 1108.9 [M+H]⁺. ¹HNMR (400 MHz, DMSO-d₆) δ 9.65 (s, 1H), 8.97 (s, 1H), 8.92 (s, 1H), 8.81-8.78 (m, 1H), 8.76-8.65 (m, 1H), 8.64 (s, 1H), 8.50-8.45 (m, 2H), 8.27-8.24 (m, 1H), 8.08 (d, J=8.2 Hz, 1H), 7.94-7.91 (m, 1H), 7.89-7.84 (m, 1H), 7.63-7.57 (m, 1H), 7.44-7.36 (m, 3H), 7.28 (d, J=6.5 Hz, 1H), 6.99 (s, 1H), 6.94 (d, J=8.7 Hz, 1H), 4.85-4.73 (m, 2H), 4.59 (d, J=9.2 Hz, 1H), 4.53-4.50 (m, 4H), 4.35 (s, 1H), 4.30-4.20 (m, 3H), 4.05-4.03 (m, 2H), 3.64-3.61 (m, 2H), 3.35-3.31 (m, 2H), 2.68 (s, 3H), 2.45 (s, 3H), 2.07-1.94 (m, 2H), 1.78-1.74 (m, 2H), 1.61-1.57 (m, 2H), 1.51-1.47 (m, 2H), 1.40-1.34 (m, 4H), 1.25-1.23 (m, 2H), 0.95 (s, 9H).

### Example A133. 4-amino-N-((S)-6-(6-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) pentyl)carbamoyl)pyridin-3-yl)-2,3-dihydrobenzofuran-3-yl)-N,1-dimethyl-1H-pyrazolo[4,3-c]quinoline-8-carboxamide

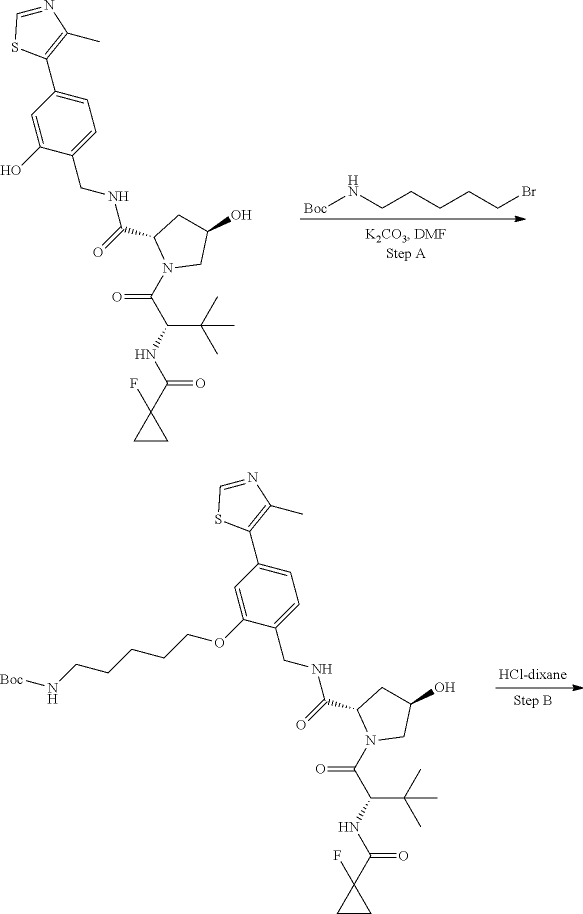 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)NCCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCCNC(=O)OC(C)(C)C)c1]

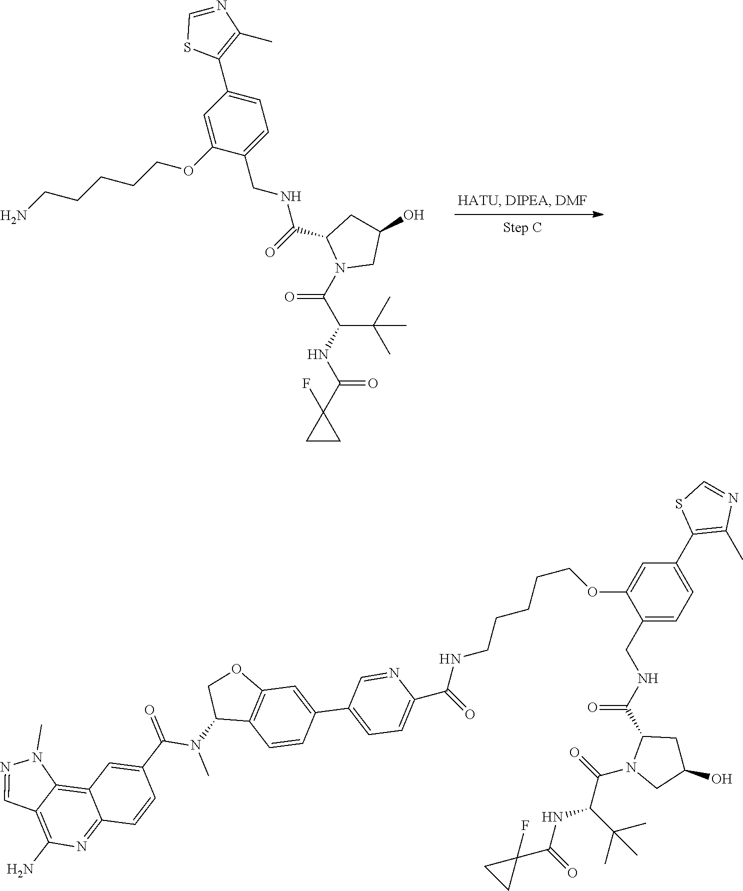 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCN)c1 ; Cc1ncsc1-c1ccc(CNC(=O)[C@@H]2C[C@@H](O)CN2C(=O)[C@@H](NC(=O)C2(F)CC2)C(C)(C)C)c(OCCCCCNC(=O)c2ccc(-c3ccc4c(c3)OC[C@H]4N(C)C(=O)c3ccc4nc(N)c5cnn(C)c5c4c3)cn2)c1]

Example A133 was synthesized following the same procedures as described in Example A23 (5.5 mg, 5% yield over 3 steps) as a white solid. LC-MS (ESI): mass calcd. for C₅₈H₆₄FN₁₁O₈S, 1093.46; m/z found, 1094.46 [M+H]⁺. ¹H NMR (400 MHz, DMSO-d₆) δ 8.97 (s, 1H), 8.92 (d, J=1.8 Hz, 1H), 8.83 (t, J=6.1 Hz, 1H), 8.49 (t, J=6.0 Hz, 1H), 8.35 (s, 1H), 8.29 (s, 1H), 8.27 (dd, J=8.3, 2.2 Hz, 1H), 8.08 (d, J=8.2 Hz, 1H), 7.70-7.63 (m, 2H), 7.58 (d, J=6.9 Hz, 1H), 7.43-7.38 (m, 2H), 7.35 (s, 1H), 7.33-7.23 (m, 3H), 7.02-6.99 (m, 1H), 6.94 (dd, J=7.8, 1.4 Hz, 1H), 5.25-5.09 (m, 1H), 4.83-4.72 (m, 2H), 4.60 (d, J=9.2 Hz, 1H), 4.52 (t, J=8.2 Hz, 1H), 4.44 (s, 3H), 4.36-4.33 (m, 1H), 4.32-4.27 (m, 1H), 4.23-4.16 (m, 1H), 4.06 (t, J=6.2 Hz, 2H), 3.64-3.60 (m, 2H), 3.50-3.47 (m, 1H), 3.38-3.35 (m, 2H), 2.72 (s, 3H), 2.46 (s, 3H), 2.11-2.04 (m, 1H), 1.95-1.87 (m, 1H), 1.84-1.77 (m, 2H), 1.68-1.61 (m, 2H), 1.54-1.47 (m, 2H), 1.40-1.31 (m, 2H), 1.24-1.20 (m, 2H), 0.95 (s, 9H).

### Example A134. 2-amino-N-(S)-6-(6-(4-(4-(2-(2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)piperidine-1-carbonyl)pyridin-3-yl)-2,3-dihydrobenzofuran-3-yl)-N,3-dimethylquinoline-6-carboxamide

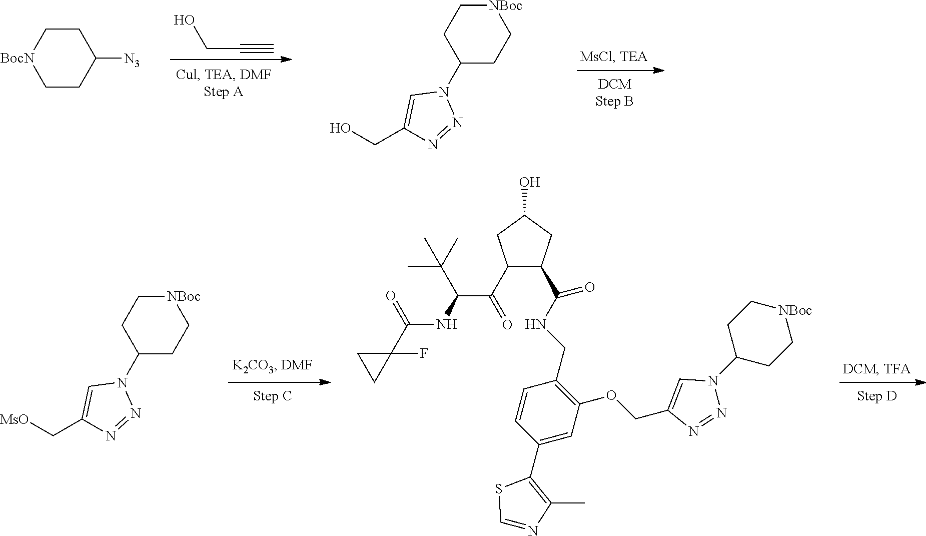

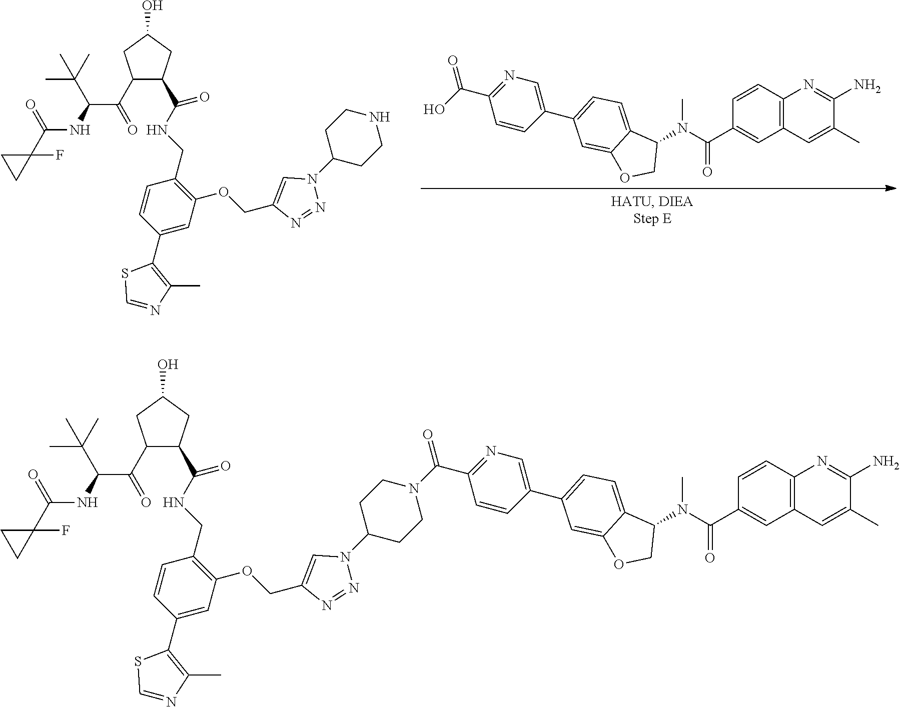 [SMILES: Cc1ncsc1-c1ccc(CNC(=O)[C@@H]2C[C@@H](O)CC2C(=O)[C@@H](NC(=O)C2(F)CC2)C(C)(C)C)c(OCc2cn(C3CCNCC3)nn2)c1 ; Cc1cc2cc(C(=O)N(C)[C@@H]3COc4cc(-c5ccc(C(=O)O)nc5)ccc43)ccc2nc1N ; Cc1cc2cc(C(=O)N(C)[C@@H]3COc4cc(-c5ccc(C(=O)N6CCC(n7cc(COc8cc(-c9scnc9C)ccc8CNC(=O)[C@@H]8C[C@@H](O)CC8C(=O)[C@@H](NC(=O)C8(F)CC8)C(C)(C)C)nn7)CC6)nc5)ccc43)ccc2nc1N]

Step A: tert-butyl 4-(4-(hydroxymethyl)-1H-1,2,3-triazol-1-yl)piperidine-1-carboxylate

To a solution of prop-2-yn-1-ol (50 mg, 892 μmol, 1 eq.) in DMF (3 mL) was added tert-butyl 4-azidopiperidine-1-carboxylate (202 mg, 892 μmol, 1 eq.), CuI (34 mg, 178 μmol, 0.2 eq.) and TEA (181 mg, 1.78 mmol, 2 eq.) at room temperature. The mixture was then stirred at room temperature for 16 hours under N₂ atmosphere. The resulting solution was diluted with 30 mL of water and extracted with EtOAc (50 mL×3). The combined organic layers were washed with brine, dried over anhydrous Na₂SO₄ and concentrated under reduced pressure. The residue was purified by column chromatography (silica gel, 0% MeOH in DCM~7% MeOH in DCM) to afford tert-butyl 4-(4-(hydroxymethyl)-1H-1,2,3-triazol-1-yl)piperidine-1-carboxylate (175 mg, 70% yield) as a yellow oil. LC-MS (ESI): mass calcd. For C₁₃H₂₂N₄O₃, 282.2; m/z found, 283.2 [M+H]⁺.

Steps B-E: 2-amino-N-(S)-6-16-(4-(4-(2-(((25, 4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)piperidine-1-carbonyl)pyridin-3-yl)-2,3-dihydrobenzofuran-3-yl)-N,3-dimethylquinoline-6-carboxamide

Example A134 was synthesized following the same procedures as described in Example A13 (4.9 mg, 3% yield over 4 steps) as a white solid. LC-MS (ESI): mass calcd. for C₆₀H₆₈FN₁₂O₈S, 1132.5/z found, 1133.3 [M+H]⁺. ¹H NMR (400 MHz, DMSO-d₆) δ 8.99 (s, 1H), 8.92 (s, 1H), 8.56-8.46 (m, 1H), 8.41 (s, 1H), 8.26-8.20 (m, 1H), 7.88 (s, 1H), 7.82 (s, 1H), 7.71 (d, J=8.2 Hz, 1H), 7.59 (d, J=8.4 Hz, 1H), 7.54 (d, J=7.4 Hz, 2H), 7.44-7.37 (m, 2H), 7.34 (s, 1H), 7.30-7.23 (m, 2H), 6.99 (d, J=8.1 Hz, 1H), 6.91-6.53 (m, 2H), 5.30-5.24 (m, 2H), 5.16 (d, J=3.5 Hz, 1H), 4.93-4.86 (m, 1H), 4.83-4.62 (m, 3H), 4.61-4.57 (m, 1H), 4.53-4.47 (m, 1H), 4.36-4.31 (m, 1H), 4.29-4.18 (m, 2H), 3.97 (d, J=12.9 Hz, 1H), 3.67-3.57 (m, 2H), 3.39-3.35 (m, 1H), 3.12-3.04 (m, 1H), 2.67 (s, 3H), 2.45 (s, 3H), 2.27-2.21 (m, 4H), 2.12-1.98 (m, 4H), 1.93-1.83 (m, 1H), 1.41-1.31 (m, 2H), 1.24-1.20 (m, 2H), 0.93 (s, 9H).

### Example A135. N-(cyclopropylmethyl)-N-((S)-6-(6-((1-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)piperidin-4-yl)carbamoyl)pyridin-3-yl)-2,3-dihydrobenzofuran-3-yl)-3-methylquinoline-6-carboxamide

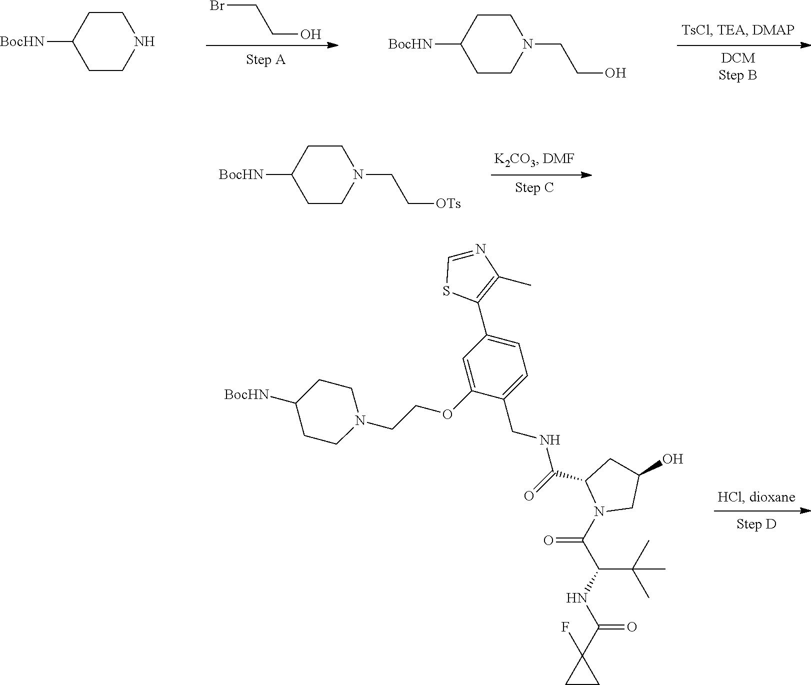

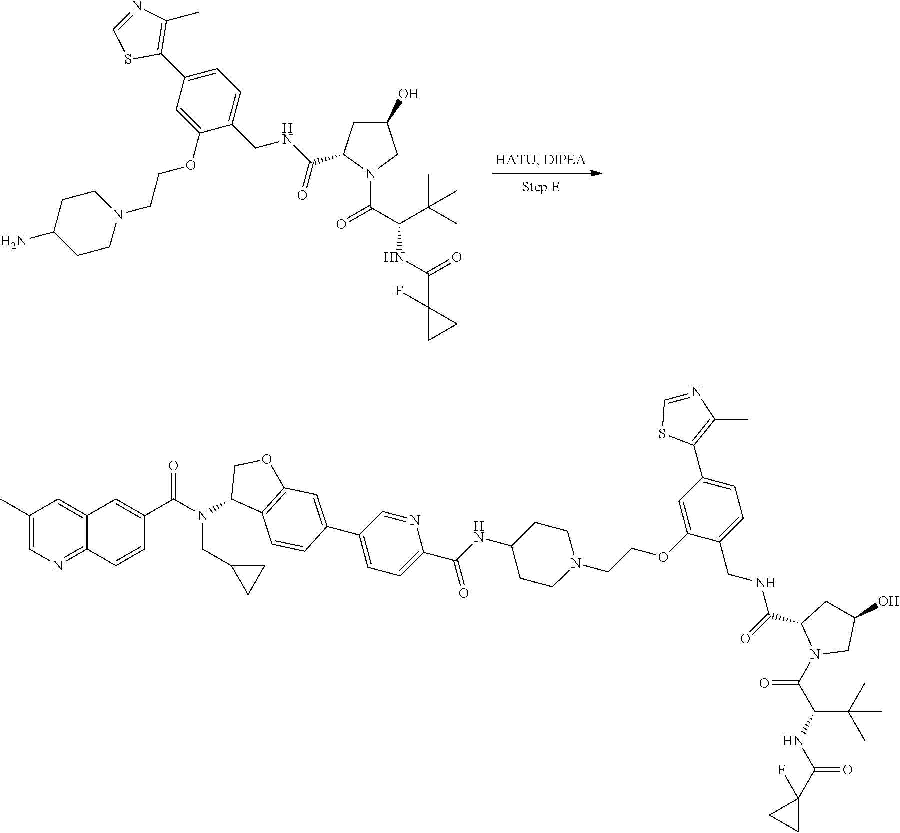 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCC(N)CC2)c1 ; Cc1cnc2ccc(C(=O)N(CC3CC3)[C@@H]3COc4cc(-c5ccc(C(=O)NC6CCN(CCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2c1]

Example A135 was synthesized following the same procedures as described in Example A29 (9.7 mg, 7% yield over 5 steps) as a white solid. LC-MS (ESI): mass calcd, for C₆₂H₇₀FN₉O₈S, 1119.51; m/z found, 1120.7 [M+H]⁺. ¹HNMR (400 MHz, DMSO-d₆) δ 8.99 (s, 1H), 8.93 (s, 1H), 8.84 (d, J=1.9 Hz, 1H), 8.64 (s, 1H), 8.57-8.46 (m, 1H), 8.31-8.20 (m, 2H), 8.11-8.01 (m, 3H), 7.76 (d, J=8.1 Hz, 1H), 7.65-7.60 (m, 1H), 7.46-7.36 (m, 2H), 7.34-7.24 (m, 2H), 7.06 (s, 1H), 7.01-6.93 (m, 1H), 5.23-5.15 (m, 1H), 4.77 (s, 2H), 4.59 (d, J=9.2 Hz, 1H), 4.55-4.49 (m, 1H), 4.36 (s, 1H), 4.32-4.14 (m, 4H), 4.01-3.75 (m, 2H), 3.68-3.60 (m, 2H), 3.50-3.46 (m, 1H), 3.20-3.05 (m, 5H), 3.00-2.87 (m, 2H), 2.49 (s, 3H), 2.47 (s, 3H), 2.13-2.06 (m, 1H), 1.96-1.89 (m, 1H), 1.88-1.70 (m, 4H), 1.40-1.31 (m, 2H), 1.24-1.18 (m, 2H), 0.95 (s, 9H), 0.93-0.77 (m, 2H), 0.41-0.26 (m, 2H), −0.16-0.26 (m, 1H).

### Example A136. N-(cyclopropylmethyl)-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)phenyl)carbamoyl)pyridin-3-yl)-2,3-dihydrobenzofuran-3-yl)-3-methylquinoline-6-carboxamide

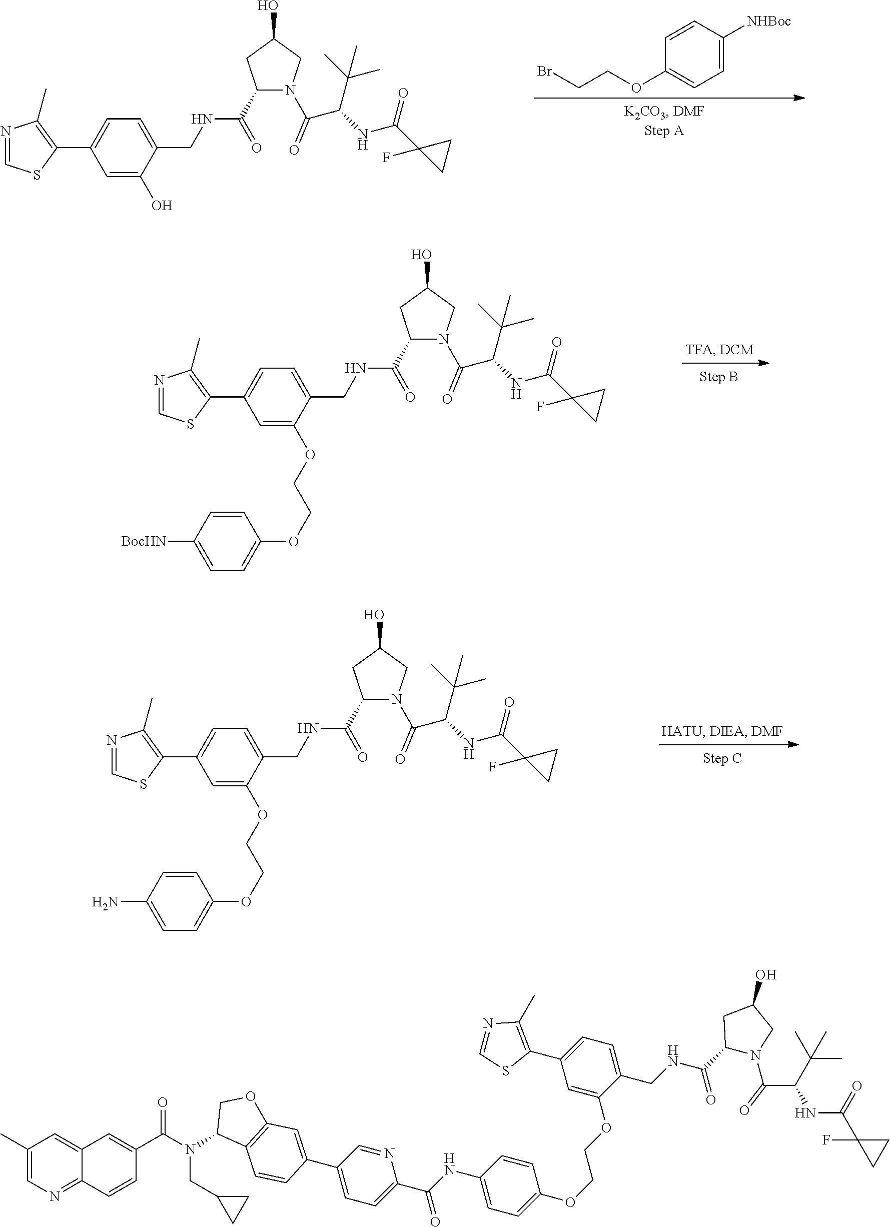 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; CC(C)(C)OC(=O)Nc1ccc(OCCBr)cc1 ; Cc1ncsc1-c1ccc(CNC(=O)[C@@H]2C[C@@H](O)CN2C(=O)[C@@H](NC(=O)C2(F)CC2)C(C)(C)C)c(OCCOc2ccc(NC(=O)OC(C)(C)C)cc2)c1 ; Cc1ncsc1-c1ccc(CNC(=O)[C@@H]2C[C@@H](O)CN2C(=O)[C@@H](NC(=O)C2(F)CC2)C(C)(C)C)c(OCCOc2ccc(N)cc2)c1 ; Cc1cnc2ccc(C(=O)N(CC3CC3)[C@@H]3COc4cc(-c5ccc(C(=O)Nc6ccc(OCCOc7cc(-c8scnc8C)ccc7CNC(=O)[C@@H]7C[C@@H](O)CN7C(=O)[C@@H](NC(=O)C7(F)CC7)C(C)(C)C)cc6)nc5)ccc43)cc2c1]

Example A136 was synthesized following the same procedures as described in Example A23 (3.3 mg, 6% yield over 3 steps) as a white solid. LC-MS (ESI): mass calcd, for C₆₃H₆₅FN₈O₉S, 1128.46; m/z found, 1129.5 [M+H]⁺. ¹HNMR (400 MHz, DMSO-d₆) δ 10.78 (s, 1H), 9.22 (s, 1H), 9.19 (s, 1H), 9.07-9.05 (m, 1H), 8.73-8.67 (m, 1H), 8.56-8.53 (m, 1H), 8.47 (s, 1H), 8.40 (d, J=8.2 Hz, 1H), 8.29-8.25 (m, 2H), 8.06 (d, J=9.0 Hz, 2H), 8.00-7.95 (m, 1H), 7.84 (d, J=7.8 Hz, 1H), 7.65-7.61 (m, 2H), 7.56 (s, 1H), 7.52-7.47 (m, 1H), 7.31 (s, 1H), 7.25-7.21 (m, 2H), 7.20-7.18 (m, 1H), 5.11-4.94 (m, 2H), 4.80 (d, J=9.2 Hz, 1H), 4.74-4.69 (m, 1H), 4.64-4.60 (m, 2H), 4.58-4.53 (m, 3H), 4.52-4.40 (m, 2H), 3.86-3.79 (m, 2H), 3.68-3.65 (m, 1H), 3.40-3.36 (m, 1H), 3.31-3.25 (m, 1H), 2.71 (s, 3H), 2.68 (s, 3H), 2.32-2.26 (m, 1H), 2.15-2.06 (m, 1H), 1.61-1.52 (m, 2H), 1.44-1.40 (m, 2H), 1.15 (s, 9H), 1.14-0.98 (m, 2H), 0.60-0.48 (m, 2H), 0.04-0.03 (m, 1H).

### Example A137. 2-amino-N-((S)-6-(6-(4-(4-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)phenyl)piperazine-1-carbonyl)pyridin-3-yl)-2,3-dihydrobenzofuran-3-yl)-N,3-dimethylquinoline-6-carboxamide

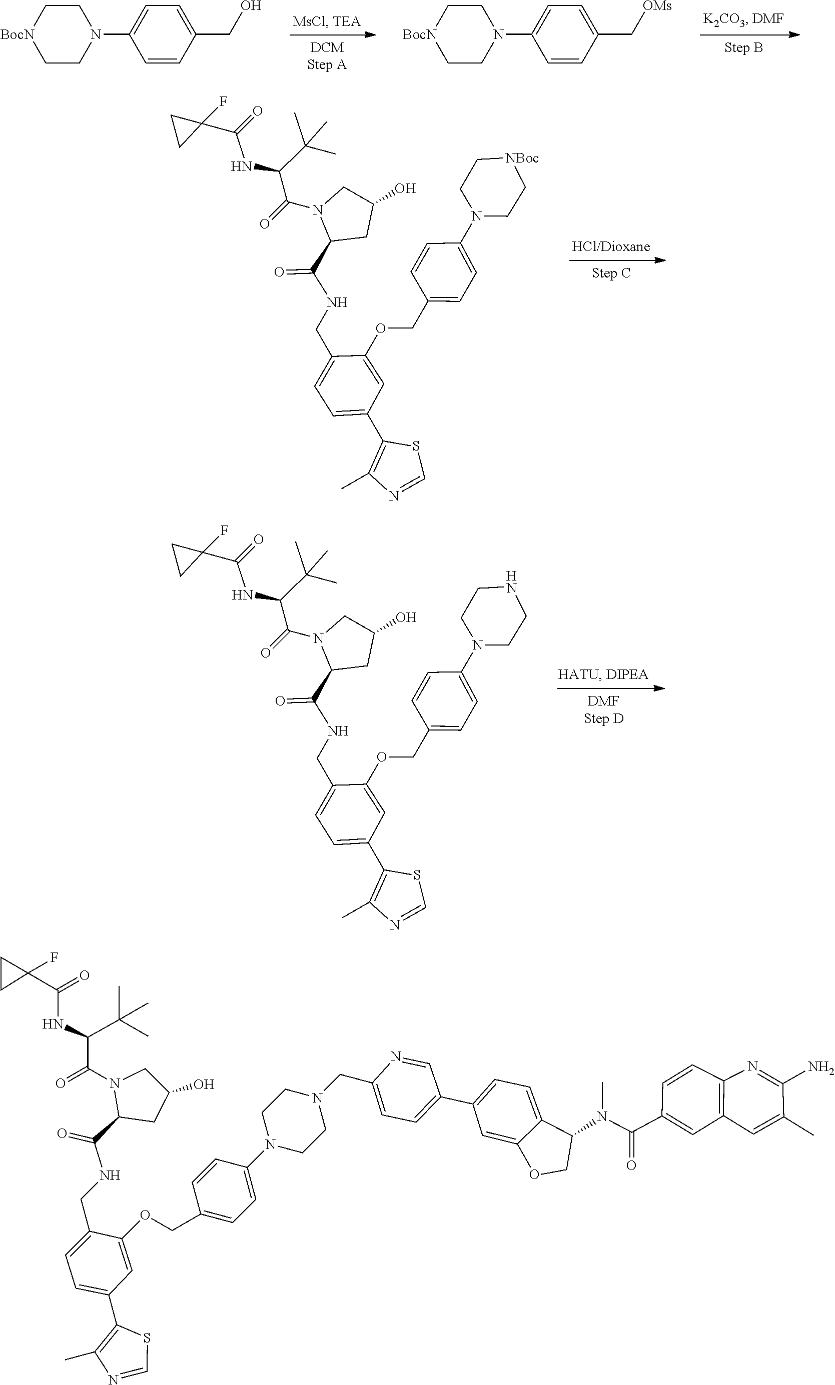

Example A137 was synthesized following the same procedures as described in Example A29 (2 mg, 1% yield over 4 steps) as a white solid. LC-MS (ESI): mass calcd, for C₆₃H₆₇FN100&S, 1142.48; m/z found, 1143.5 [M+H]⁺. ¹HNMR (400 MHz, DMSO-d₆) δ 8.98 (s, 1H), 8.94 (s, 1H), 8.50 (s, 1H), 8.24 (d, J=8.2 Hz, 1H), 7.96 (s, 1H), 7.86 (s, 1H), 7.71 (d, J=8.3 Hz, 1H), 7.66-7.62 (m, 1H), 7.62-7.50 (m, 3H), 7.44-7.25 (m, 8H), 7.09 (s, 1H), 7.00 (d, J=8.5 Hz, 2H), 6.95 (d, J=7.4 Hz, 1H), 5.16 (d, J=3.2 Hz, 1H), 5.11 (s, 2H), 4.76-4.65 (m, 2H), 4.61-4.57 (m, 1H), 4.54-4.47 (m, 1H), 4.36-4.22 (m, 3H), 3.84-3.80 (m, 2H), 3.66-3.59 (m, 4H), 3.47-3.45 (m, 1H), 3.28-3.26 (m, 2H), 3.19-3.16 (m, 2H), 2.67 (s, 3H), 2.40 (s, 3H), 2.26 (s, 3H), 2.06-2.00 (m, 1H), 1.94-1.89 (m, 1H), 1.39-1.33 (m, 2H), 1.24-1.21 (m, 2H), 0.96 (s, 9H).

### Example A138. 2-amino-N-((3S)-6-(6-(4-((5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl) hexahydropyrrolo[3,4-c]pyrrol-2 (1H)-yl)methyl)piperidine-1-carbonyl)pyridin-3-yl)-2,3-dihydrobenzofuran-3-yl)-N,3-dimethylquinoline-6-carboxamide

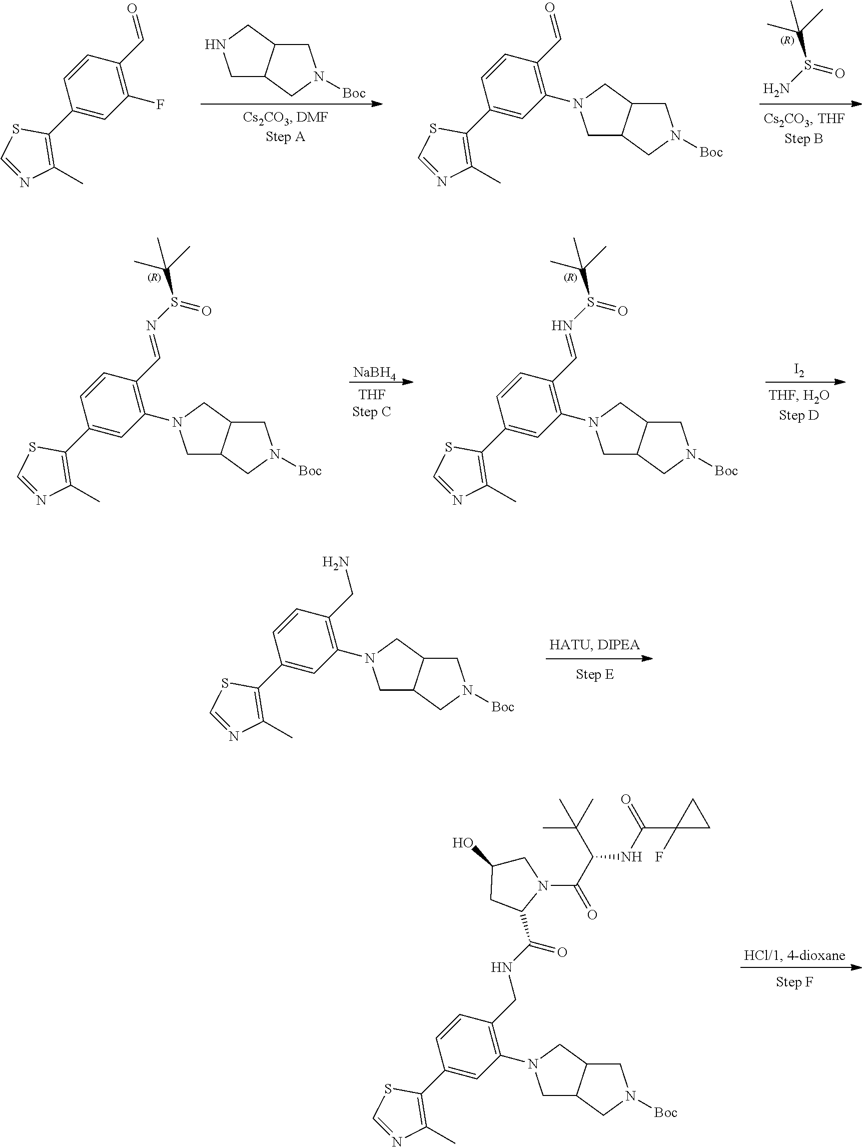

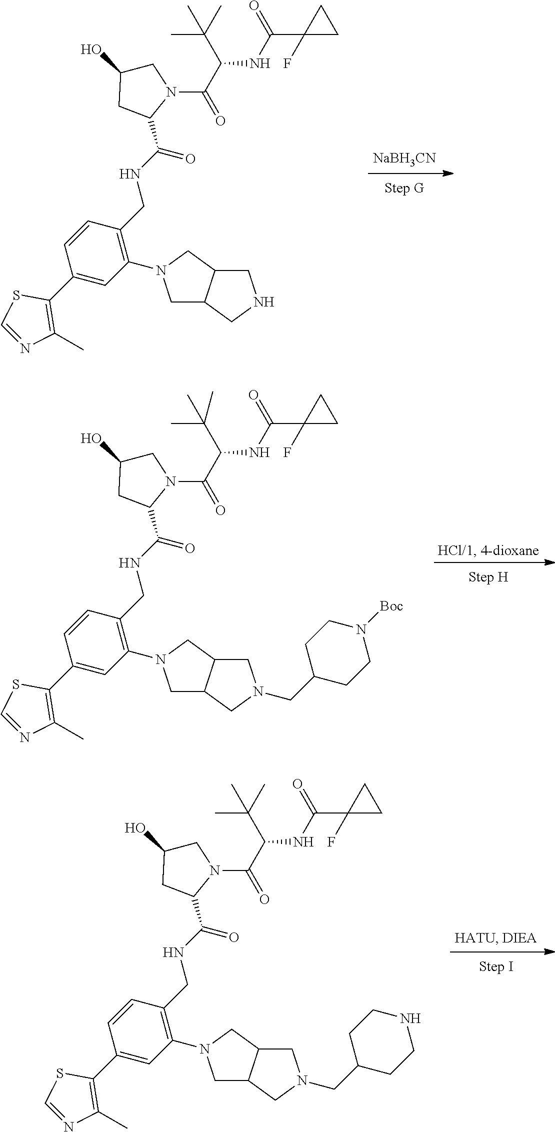

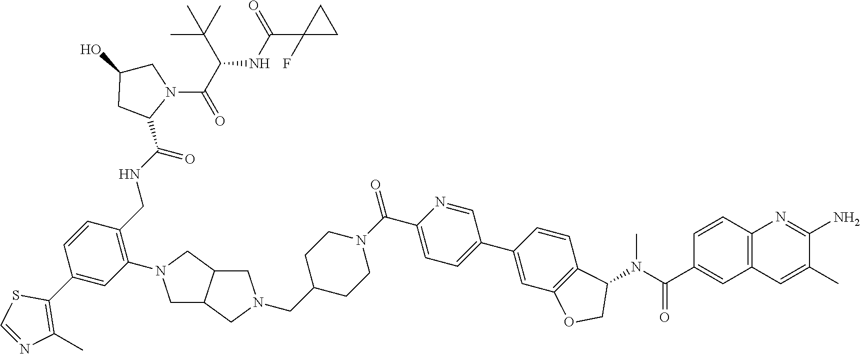

Example A138 was synthesized following the same procedures as described in Example A126 (3.8 mg, 5% yield over 9 steps) as a white solid. LC-MS (ESI): mass calcd. for C₆₄H₇₄FN₁₁O₇S, 1159.55; m/z found, 1160.55 [M+H]⁺. ¹HNMR (400 MHz, MeOD-d) δ 8.93 (s, 1H), 8.86 (s, 1H), 8.29 (s, 1H), 8.21 (d, J=8.5 Hz, 1H), 8.03 (d, J=1.5 Hz, 1H), 7.92-7.86 (m, 1H), 7.76 (s, 1H), 7.69 (d, J=8.1 Hz, 1H), 7.58-7.54 (m, 1H), 7.50-7.43 (m, 1H), 7.38-7.33 (m, 1H), 7.28-7.22 (m, 3H), 4.76-4.70 (m, 3H), 4.62-4.48 (m, 4H), 4.07-4.02 (m, 1H), 3.99-3.75 (m, 4H), 3.52-3.47 (m, 1H), 3.27-3.24 (m, 2H), 3.20-3.07 (m, 6H), 3.01-2.93 (m, 2H), 2.87-2.69 (m, 4H), 2.49 (s, 3H), 2.42 (s, 3H), 2.28-2.24 (m, 1H), 2.22-2.16 (m, 1H), 2.12-1.97 (m, 3H), 1.87-1.77 (m, 1H), 1.49-1.40 (m, 2H), 1.38-1.31 (m, 5H), 1.04-0.96 (m, 9H).

### Example A139. 2-amino-N-((S)-6-(6-(9-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-3,9-diazaspiro[5.5]undecane-3-carbonyl)pyridin-3-yl)-2,3-dihydrobenzofuran-3-yl)-N,3-dimethylquinoline-6-carboxamide

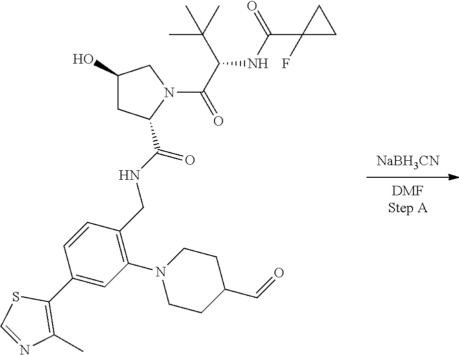 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(C=O)CC2)c1 ; "DMFStep A" ; "NaBH3CN"]

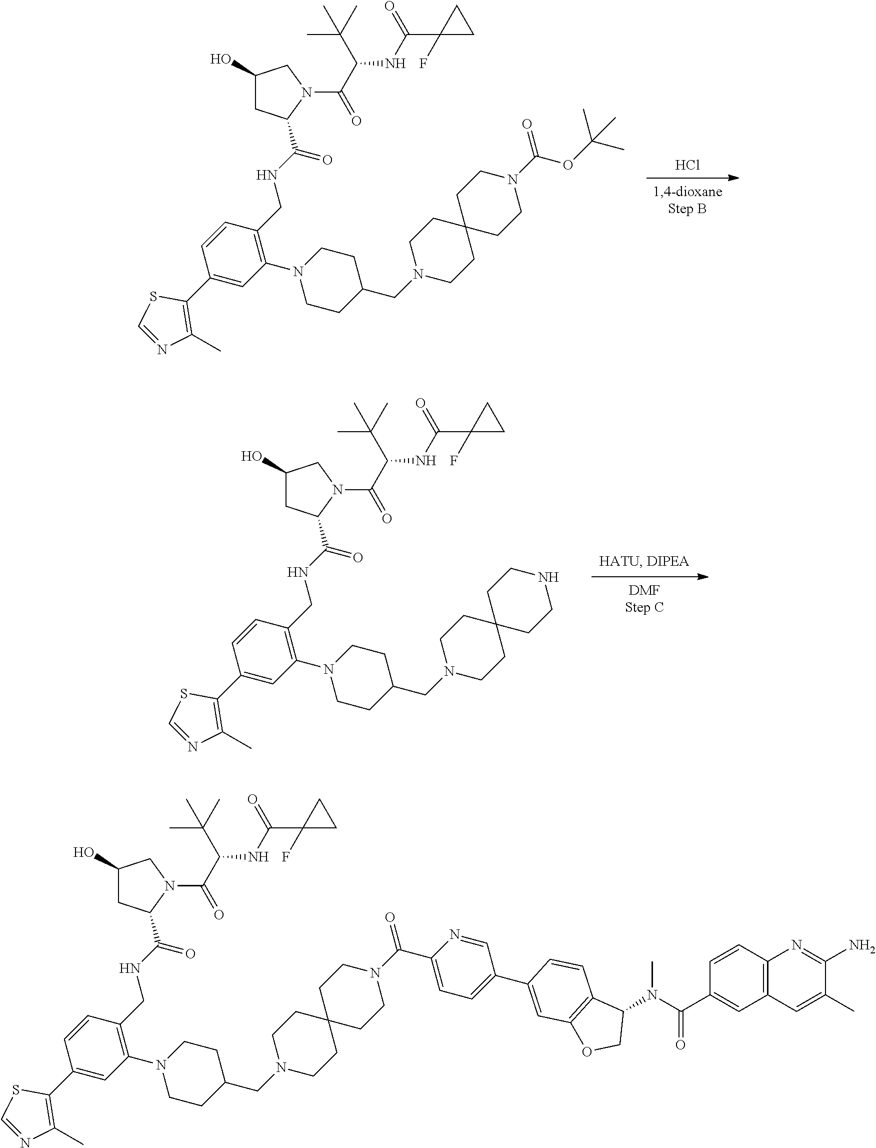

Example A139 was synthesized following the same procedures as described in Example A107 (1.0 mg, 3% yield over 5 steps) as a white solid. LC-MS (ESI): mass calcd. for C₆₇H₈₀FN₁₁O₇S, 1202.51; m/z found, 1203 [M+H]⁺. ¹HNMR (400 MHz, DMSO-d₆) δ 8.99 (s, 1H), 8.95-8.82 (m, 2H), 8.72 (s, 1H), 8.59-8.40 (m, 1H), 8.32 (s, 1H), 8.26-8.19 (m, 1H), 8.05 (s, 1H), 7.91-7.84 (m, 1H), 7.83-7.73 (m, 1H), 7.68-7.61 (m, 1H), 7.59-7.54 (m, 1H), 7.47-7.32 (m, 3H), 7.26 (s, 1H), 7.17-7.10 (m, 2H), 4.97-4.75 (m, 1H), 4.75-4.57 (m, 3H), 4.56-4.41 (m, 2H), 4.39-4.31 (m, 3H), 3.79-3.75 (m, 4H), 3.39-3.37 (m, 3H), 3.12-3.07 (m, 5H), 2.70-2.63 (m, 6H), 2.47 (s, 3H), 2.34 (s, 3H), 2.14-2.04 (m, 2H), 2.00-1.83 (m, 7H), 1.74-1.70 (m, 1H), 1.65-1.60 (m, 2H), 1.46-1.36 (m, 5H), 1.25-1.21 (m, 2H), 0,98 (s, 9H).

### Example A140. 2-amino-N-((S)-6-(6-(4-((2′-(((2S,4R)-1-((S)-2-(1-fluorocyclopropane-1-carboxamido)-3,3-dimethylbutanoyl)-4-hydroxypyrrolidine-2-carboxamido)methyl)-5′-(4-methylthiazol-5-yl)-[1,1′-biphenyl]-4-yl)methyl)piperazine-1-carbonyl)pyridin-3-yl)-2,3-dihydrobenzofuran-3-yl)-N,3-dimethylquinoline-6-carboxamide

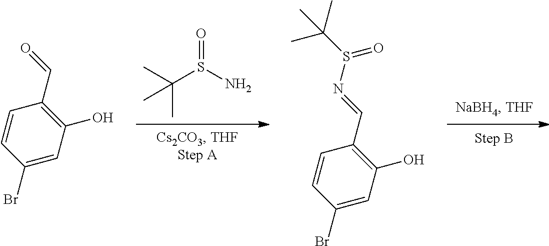

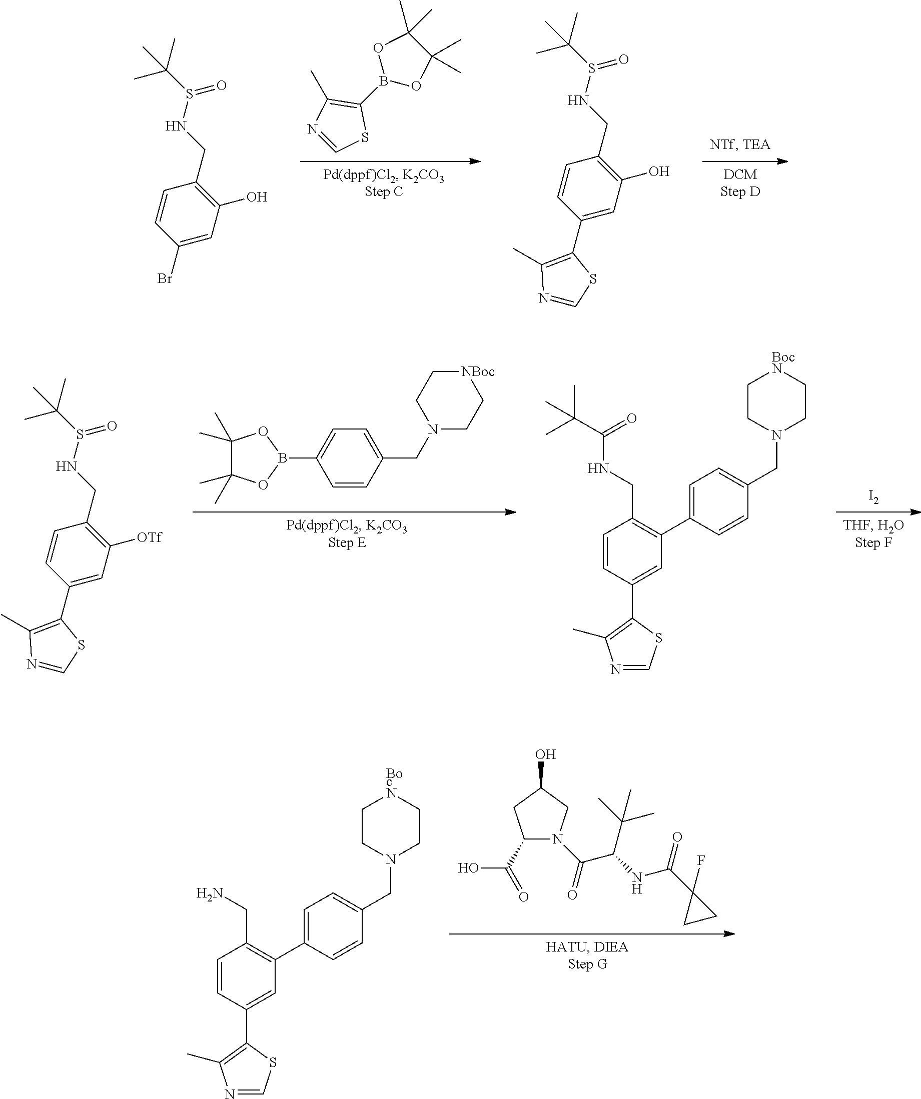

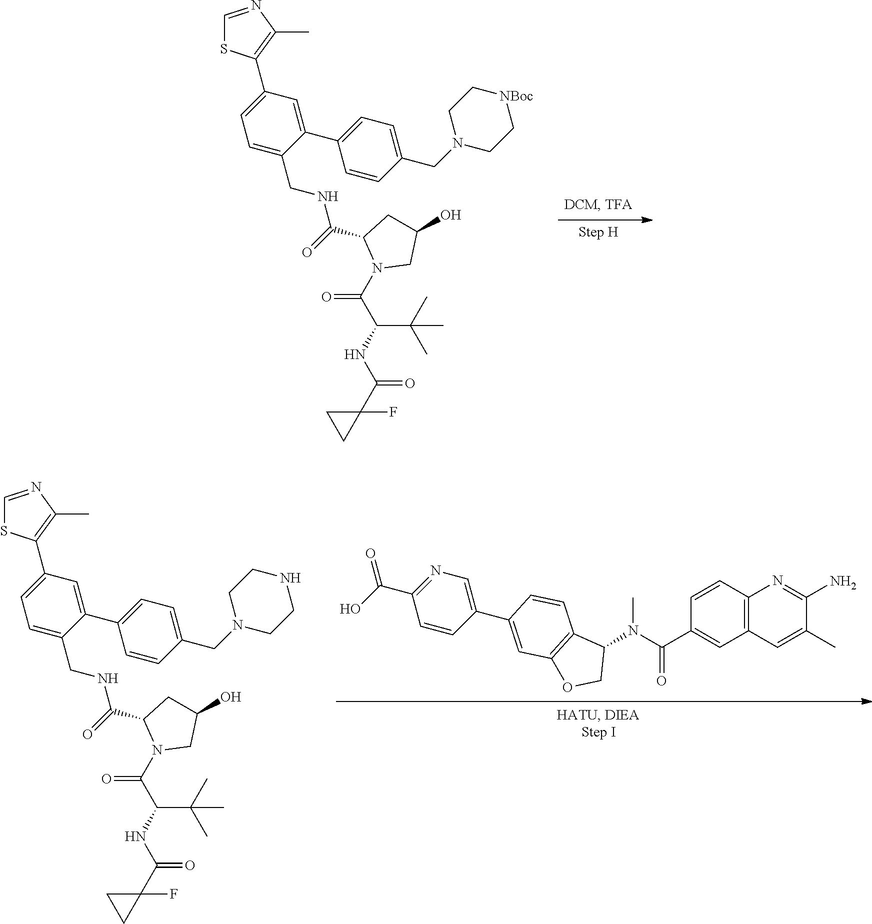

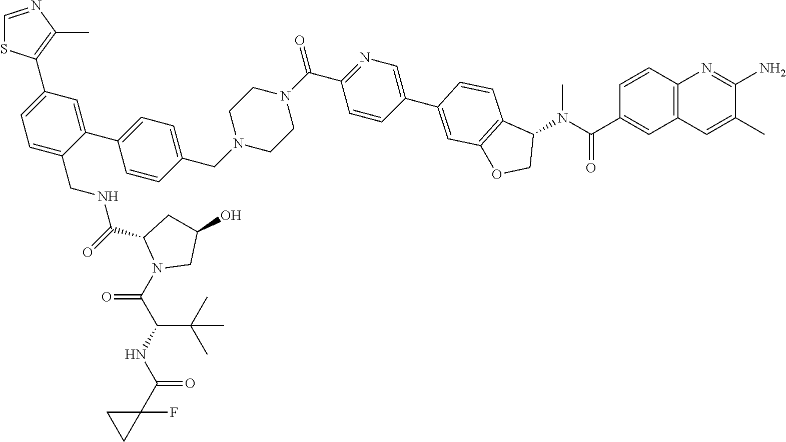 [SMILES: Cc1cc2cc(C(=O)N(C)[C@@H]3COc4cc(-c5ccc(C(=O)N6CCN(Cc7ccc(-c8cc(-c9scnc9C)ccc8CNC(=O)[C@@H]8C[C@@H](O)CN8C(=O)[C@@H](NC(=O)C8(F)CC8)C(C)(C)C)cc7)CC6)nc5)ccc43)ccc2nc1N]

Step A: (E)-N-(4-bromo-2-hydroxybenzylidene)-2-methylpropane-2-sulfinamide

To a solution of 4-bromo-2-hydroxybenzaldehyde (5 g, 24.9 mmol, 1.0 eq.) in THF (50 mL) was added 2-methylpropane-2-sulfinamide (4.52 g, 37.3 mmol, 1.5 eq.) and Cs₂CO₃ (20.3 g, 62.2 mmol, 2.5 eq.) at room temperature. The mixture was then stirred at 50° C. for 3 hours under N2 atmosphere. The resulting solution was diluted with 30 mL of water and extracted with EtOAc (80 mL×2). The combined organic layers were washed with brine, dried over anhydrous Na₂SO₄ and concentrated under reduced pressure. The residue was purified by column chromatography (silica gel, 0% EtOAc in PE~5% EtOAc in PE) to afford (E)-N-(4-bromo-2-hydroxybenzylidene)-2-methylpropane-2-sulfinamide (5 g, 66% yield) as a white solid. LC-MS (ESI): mass calcd. For C₁₁H₁₄BrNO₂S, 303.0/305.0; m/z found, 303.9/305.9 [M+H]⁺.

Step B: N-(4-bromo-2-hydroxybenzyl)-2-methylpropane-2-sulfinamide

To a solution of (E)-N-(4-bromo-2-hydroxybenzylidene)-2-methylpropane-2-sulfinamide (4 g, 13.1 mmol, 1.0 eq.) in THF (40 mL) was added NaBH₄ (1.49 g, 39.4 mmol, 3.0 eq.) at 0° C. The mixture was then stirred at room temperature for 2 hours under N₂ atmosphere. The resulting solution was diluted with 30 mL of water and extracted with EtOAc (50 mL×3). The combined organic layers were washed with brine, dried over anhydrous Na₂SO₄ and concentrated under reduced pressure. The residue was purified by column chromatography (silica gel, 0% EtOAc in PE~50% EtO Ac in PE) to afford N-(4-bromo-2-hydroxybenzyl)-2-methylpropane-2-sulfinamide (3.65 g, 90% yield) as a white solid. LC-MS (ESI): mass calcd. For C₁₁H₁₆BrNO₂S, 305.0/307.0; m/z found, 305.8/307.8 [M+H]⁺

Step C: N-(2-hydroxy-4-(4-methylthiazol-5-yl)benzyl)-2-methylpropane-2-sulfinamide

To a solution of N-(4-bromo-2-hydroxybenzyl)-2-methylpropane-2-sulfinamide (100 mg, 327 μmol, 1.0 eq.) in 1,4-Dioxane (2 mL) and H₂O (0.2 mL) was added 4-methyl-5-(4,4,5,5-tetramethyl-1,3,2-dioxaborolan-2-yl) thiazole (88 mg, 392 μmol, 1.2 eq.), 1,1′-Bis(diphenylphosphino) ferrocene-palladium (II) dichloride (24 mg, 32.7 μmol, 0.1 eq.) and K₂CO₃ (135 mg, 980 μmol, 3.0 eq.) at room temperature. The mixture was then stirred at 100° C. for 3 hours under N₂ atmosphere. The resulting solution was diluted with 30 mL of water and extracted with EtOAc (50 mL×3). The combined organic layers were washed with brine, dried over anhydrous Na₂SO₄ and concentrated under reduced pressure. The residue was purified by column chromatography (silica gel, 0% EtOAc in PE~70% EtOAc in PE) to afford N-(2-hydroxy-4-(4-methylthiazol-5-yl)benzyl)-2-methylpropane-2-sulfinamide (100 mg, 94% yield) as a yellow oil.

LC-MS (ESI): mass calcd. For C₁₅H₂₀N₂O₂S2, 324.1; m/z found, 325.1 [M+H]⁺.

Step D: 2-(((tert-butylsulfinyl)amino)methyl)-5-(4-methylthiazol-5-yl) phenyl trifluoromethanesulfonate

To a solution of N-(2-hydroxy-4-(4-methylthiazol-5-yl)benzyl)-2-methylpropane-2-sulfinamide (100 mg, 308 μmol, 1.0 eq.) in DCM (5 mL) was added Tf₂O (275 mg, 771 μmol, 2.5 eq.) and TEA (94 mg, 925 μmol, 3.0 eq.) at room temperature. The mixture was then stirred at room temperature for 3 hours under N₂ atmosphere. The resulting solution was diluted with 30 mL of water and extracted with EtOAc (50 mL×3). The combined organic layers were washed with brine, dried over anhydrous Na₂SO₄ and concentrated under reduced pressure. The residue was purified by column chromatography (silica gel, 0% EtOAc in PE~90% EtOAc in PE) to afford 2-(((tert-butylsulfinyl)amino)methyl)-5-(4-methylthiazol-5-yl) phenyl trifluoromethanesulfonate (100 mg, 71% yield) as a yellow oil. LC-MS (ESI): mass calcd. For C₁₆H₁₉F₃N₂O₄S₃, 456.0; m/z found, 457.1 [M+H]⁺.

Step E: tert-butyl 4-(2′-(((tert-butylsulfinyl)amino)methyl)-5′-(4-methylthiazol-5-yl)-[1,1′-biphenyl]-4-yl)methyl)piperazine-1-carboxylate

To a solution of 2-(((tert-butylsulfinyl)amino)methyl)-5-(4-methylthiazol-5-yl) phenyl trifluoromethanesulfonate (100 mg, 219 μmol, 1.0 eq.) in 1,4-Dioxane (5 mL) and H₂O (0.5 mL) was added tert-butyl 4-(4-(4,4,5,5-tetramethyl-1,3,2-dioxaborolan-2-yl)benzyl)piperazine-1-carboxylate (106 mg, 263 μmol, 1.2 eq.), 1,1′-Bis(diphenylphosphino) ferrocene-palladium (II) dichloride (16.0 mg, 21.9 μmol, 0.1 eq.) and K₂CO₃ (91 mg, 657 μmol, 3.0 eq.) at room temperature. The mixture was then stirred at 100° C. for 4 hours under N₂ atmosphere. The resulting solution was diluted with 30 mL of water and extracted with EtOAc (50 mL×3). The combined organic layers were washed with brine, dried over anhydrous Na₂SO₄ and concentrated under reduced pressure. The residue was purified by column chromatography (silica gel, 0% MeOH in DCM~7% MeOH in DCM) to afford tert-butyl 4-((2′-(((tert-butylsulfinyl)amino)methyl)-5′-(4-methylthiazol-5-yl)-[1,1′-biphenyl]-4-yl)methyl)piperazine-1-carboxylate (100 mg, 78% yield) as a yellow oil. LC-MS (ESI): mass calcd. For C31H42N₄O₃S2, 582.3; m/z found, 583.9 [M+H]⁺.

Step F-I: 2-amino-N-((S)-6-(6-(4-((2′-(((2S,4R)-1-((S)-2-(1-fluorocyclopropane-1-carboxamido)-3,3-dimethylbutanoyl)-4-hydroxypyrrolidine-2-carboxamido)methyl)-5′-(4-methylthiazol-5-yl)-[1,1′-biphenyl]-4-yl)methyl)piperazine-1-carbonyl)pyridin-3-yl)-2,3-dihydrobenzofuran-3-yl)-N,3-dimethylquinoline-6-carboxamide

Example A140 was synthesized following the same procedures as described in Example A107 (5.4 mg, 1% yield over 4 steps) as a white solid. LC-MS (ESI): mass calcd. for C₆₃H₆₇FN₁₀O₇S, 1126.5/z found, 1128.0 [M+H]⁺. ¹H NMR (400 MHz, DMSO-d₆) δ 9.02 (s, 1H), 8.93 (s, 1H), 8.56 (d, J=5.8 Hz, 3H), 8.33 (s, 1H), 8.30-8.25 (m, 1H), 8.05 (s, 1H), 7.87 (d, J=8.7 Hz, 1H), 7.77 (d, J=8.1 Hz, 2H), 7.63 (s, 1H), 7.61 (s, 2H), 7.59 (s, 2H), 7.56 (d, J=3.3 Hz, 1H), 7.52-7.48 (m, 1H), 7.40 (d, J=7.5 Hz, 1H), 7.35 (s, 1H), 7.30 (d, J=1.7 Hz, 1H), 7.26 (d, J=9.1 Hz, 1H), 4.89-4.62 (m, 3H), 4.58 (d, J=9.2 Hz, 1H), 4.51-4.43 (m, 3H), 4.33 (s, 1H), 4.30-4.18 (m, 3H), 3.65-3.57 (m, 4H), 3.48-3.39 (m, 2H), 3.29-3.18 (m, 3H), 2.67 (s, 3H), 2.48 (s, 3H), 2.34 (s, 3H), 2.09-2.02 (m, 1H), 1.88-1.79 (m, 1H), 1.41-1.31 (m, 2H), 1.24-1.19 (m, 2H), 0.95 (s, 9H).

### Example A141. 2-amino-N-((S)-6-(6-(4-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)but-3-yn-1-yl)piperazine-1-carbonyl)pyridin-3-yl)-2,3-dihydrobenzofuran-3-yl)-N,3-dimethylquinoline-6-carboxamide

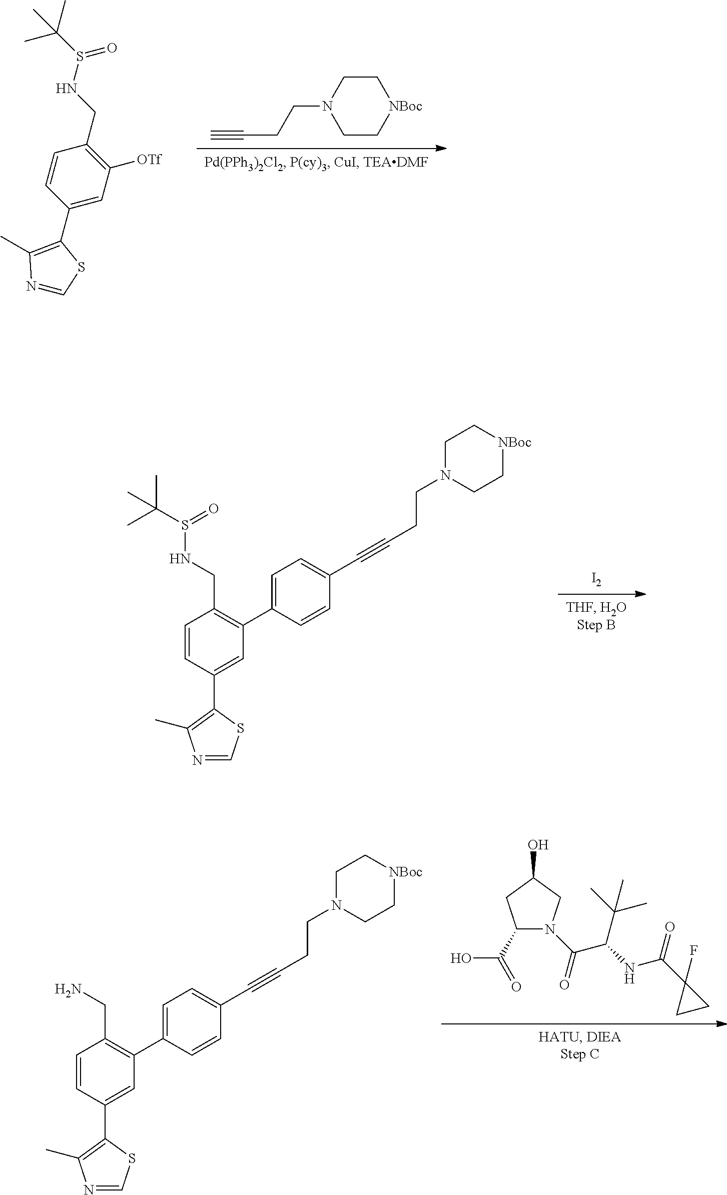

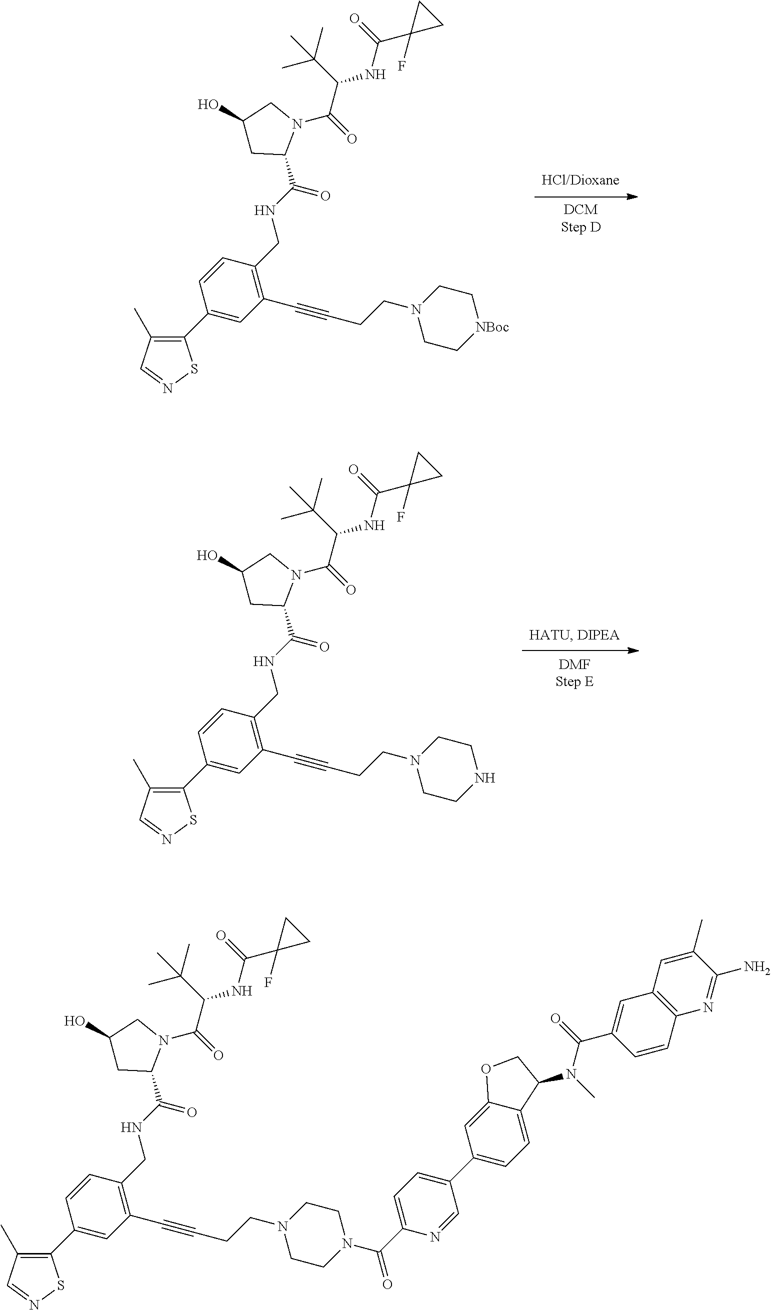 [SMILES: Cc1cnsc1-c1ccc(CNC(=O)[C@@H]2C[C@@H](O)CN2C(=O)[C@@H](NC(=O)C2(F)CC2)C(C)(C)C)c(C#CCCN2CCN(C(=O)OC(C)(C)C)CC2)c1 ; "DCMStep D" ; Cc1cnsc1-c1ccc(CNC(=O)[C@@H]2C[C@@H](O)CN2C(=O)[C@@H](NC(=O)C2(F)CC2)C(C)(C)C)c(C#CCCN2CCNCC2)c1 ; "DMFStep E" ; Cc1cc2cc(C(=O)N(C)[C@@H]3COc4cc(-c5ccc(C(=O)N6CCN(CCC#Cc7cc(-c8sncc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N]

Step A: tert-butyl 4-(4-(2-(((tert-butylsulfinyl)amino)methyl)-5-(4-methylthiazol-5-yl)phenyl)but-3-yn-1-yl)piperazine-1-carboxylate

To a solution of 2-(((tert-butylsulfinyl)amino)methyl)-5-(4-methylthiazol-5-yl) phenyl trifluoromethanesulfonate (80 mg, 175 μmol, 1 eq.) in DMF (2 mL) was added tert-butyl 4-(but-3-yn-1-yl)piperazine-1-carboxylate (84 mg, 350 μmol, 2.0 eq.), Bis-(triphenylphosphino)-palladous chloride (12 mg, 17.5 μmol, 0.1 eq.), CuI (3.3 mg, 17.5 μmol, 0.1 eq.), TEA (177 mg, 1.75 mmol, 10 eq.) and Phosphine, tricyclohexyl-(5 mg, 17.5 μmol, 0.1 eq.) at room temperature. The mixture was then stirred at 80° C. for 16 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% MeOH in DCM~5% MeOH in DCM) to afford tert-butyl 4-(4-(2-(((tert-butylsulfinyl)amino)methyl)-5-(4-methylthiazol-5-yl)phenyl)but-3-yn-1-yl)piperazine-1-carboxylate (80 mg, 84% yield) as a yellow oil. LC-MS (ESI): mass calcd. For C₂₈H40N₄O₃S2, 544.3; m/z found, 545.8 [M+H]⁺.

Steps B-E: 2-amino-N-((S)-6-(6-(4-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)but-3-yn-1-yl)piperazine-1-carbonyl)pyridin-3-yl)-2,3-dihydrobenzofuran-3-yl)-N,3-dimethylquinoline-6-carboxamide

Example A141 was synthesized following the same procedures as described in Example A23 (3 mg, 2% yield over 4 steps) as a white solid. LC-MS (ESI): mass calcd, for C₆₀H₆₅FN₁₀O₇S, 1088.5/z found, 1089.1 [M+H]⁺. ¹H NMR (400 MHz, DMSO-d₆) δ 9.01 (s, 1H), 8.92 (s, 1H), 8.89-8.66 (m, 3H), 8.32 (s, 1H), 8.27 (d, J=10.0 Hz, 1H), 8.05 (s, 1H), 7.87 (d, J=8.7 Hz, 1H), 7.77 (d, J=8.1 Hz, 2H), 7.56 (d, J=8.1 Hz, 2H), 7.48 (s, 1H), 7.45-7.37 (m, 2H), 7.35 (s, 1H), 7.28 (d, J=7.1 Hz, 1H), 4.81 (s, 1H), 4.74-4.67 (m, 2H), 4.60 (d, J=9.3 Hz, 1H), 4.54-4.49 (m, 2H), 4.45 (d, J=5.8 Hz, 1H), 4.41 (d, J=5.5 Hz, 1H), 4.38-4.33 (m, 2H), 4.32-4.22 (m, 2H), 3.65-3.61 (m, 2H), 3.52-3.42 (m, 3H), 3.36-3.14 (m, 3H), 3.06-2.99 (m, 2H), 2.66 (s, 3H), 2.44 (s, 3H), 2.33 (s, 3H), 2.14-2.07 (m, 1H), 1.95-1.87 (m, 1H), 1.41-1.32 (m, 2H), 1.24-1.20 (m, 2H), 0.95 (s, 9H).

### Example A142. 2-amino-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)phenyl)carbamoyl)pyridin-3-yl)-2,3-dihydrobenzofuran-3-yl)-N,3-dimethylquinoline-6-carboxamide

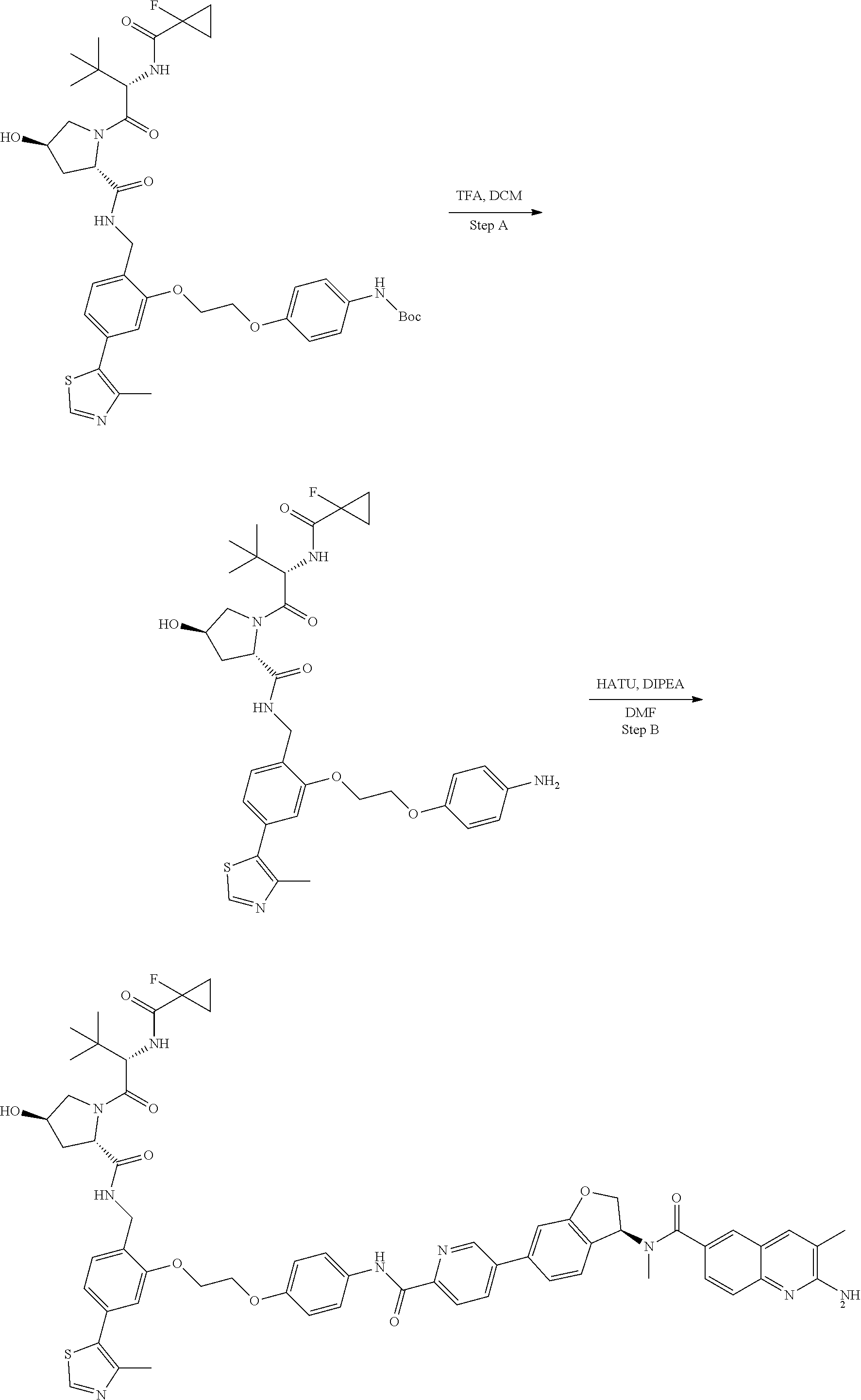 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOc2ccc(NC(=O)OC(C)(C)C)cc2)c1 ; "Step A" ; Cc1ncsc1-c1ccc(CNC(=O)[C@@H]2C[C@@H](O)CN2C(=O)[C@@H](NC(=O)C2(F)CC2)C(C)(C)C)c(OCCOc2ccc(N)cc2)c1 ; "DMFStep B" ; Cc1cc2cc(C(=O)N(C)[C@@H]3COc4cc(-c5ccc(C(=O)Nc6ccc(OCCOc7cc(-c8scnc8C)ccc7CNC(=O)[C@@H]7C[C@@H](O)CN7C(=O)[C@@H](NC(=O)C7(F)CC7)C(C)(C)C)cc6)nc5)ccc43)ccc2nc1N]

Example A142 was synthesized following the same procedures as described in Example A29 (5.9 mg, 18% yield over 2 steps) as a white solid. LC-MS (ESI): mass calcd. for C₆₀H₆₂FN₉O₉S, 1103.44; m/z found, 1104.6 [M+H]⁺. ¹H NMR (400 MHz, DMSO-d₆) δ 10.59 (s, 1H), 9.02 (s, 1H), 8.99 (s, 1H), 8.53-8.47 (m, 1H), 8.37-8.32 (m, 1H), 8.21 (d, J=8.2 Hz, 1H), 7.96 (s, 1H), 7.87 (s, 2H), 7.84 (s, 1H), 7.65 (d, J=8.3 Hz, 1H), 7.61-7.55 (m, 2H), 7.43 (d, J=7.9 Hz, 2H), 7.39 (s, 1H), 7.31-7.27 (m, 1H), 7.11 (s, 1H), 7.02 (d, J=9.0 Hz, 2H), 6.99 (d, J=8.0 Hz, 1H), 5.19-5.13 (m, 1H), 4.83-4.69 (m, 2H), 4.59 (d, J=9.2 Hz, 1H), 4.51 (t, J=8.2 Hz, 1H), 4.44-4.40 (m, 2H), 4.38-4.33 (m, 3H), 4.31-4.17 (m, 2H), 3.65-3.59 (m, 2H), 3.47-3.44 (m, 1H), 2.68 (s, 3H), 2.47 (s, 3H), 2.26 (s, 3H), 2.11-2.05 (m, 1H), 1.95-1.87 (m, 1H), 1.40-1.32 (m, 2H), 1.24-1.20 (m, 2H), 0.95 (s, 9H).

### Example A143. N1-(5-carbamoylpyridin-3-yl)-N2-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N2-methyloxalamide

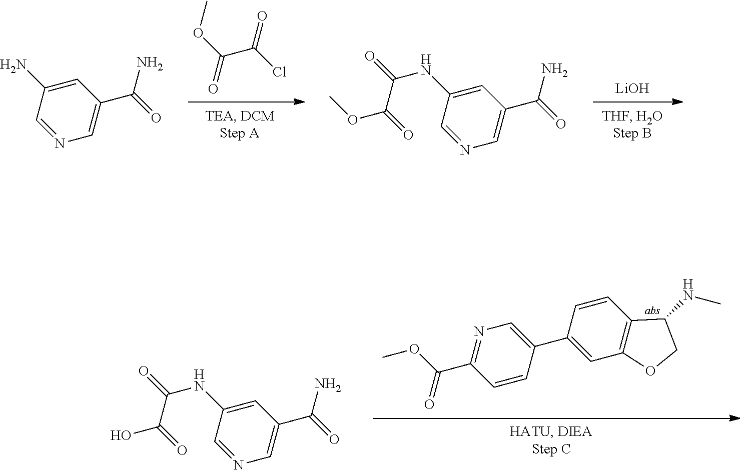

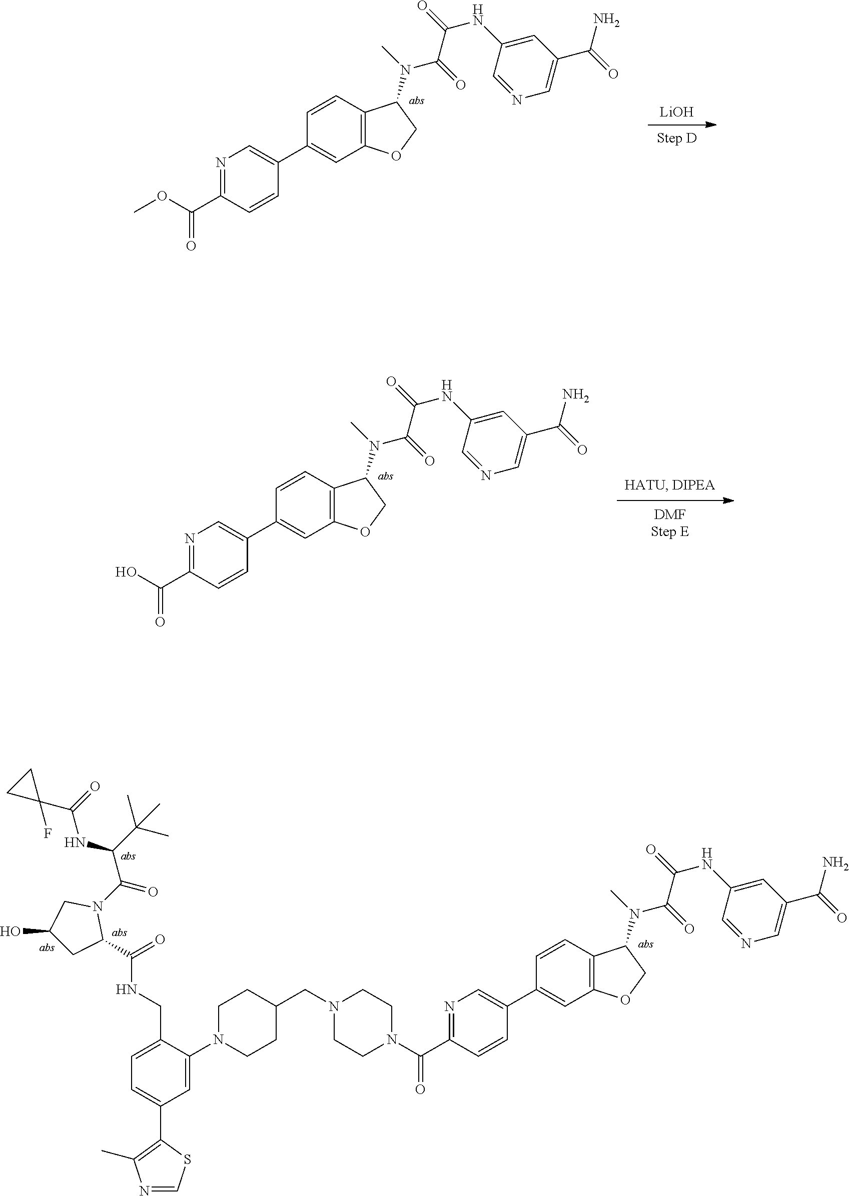 [SMILES: COC(=O)c1ccc(-c2ccc3c(c2)OC[C@H]3N(C)C(=O)C(=O)Nc2cncc(C(N)=O)c2)cn1 ; "LiOH" ; "Step D" ; CN(C(=O)C(=O)Nc1cncc(C(N)=O)c1)[C@@H]1COc2cc(-c3ccc(C(=O)O)nc3)ccc21 ; "DMFStep E" ; Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)C(=O)Nc5cncc(C(N)=O)c5)cn4)CC3)CC2)c1]

Example A143 was synthesized following the same procedures as described in Example A44 (1.8 mg, 3% yield over 5 steps) as a white solid. LC-MS (ESI): mass calcd. for C₅₉H₆₉FN₁₂O₉S, 1141.33; m/z found, 1142 [M+H]⁺.

### Example A144. N-(cyclopropylmethyl)-N-((R)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-3-methylquinoline-6-carboxamide

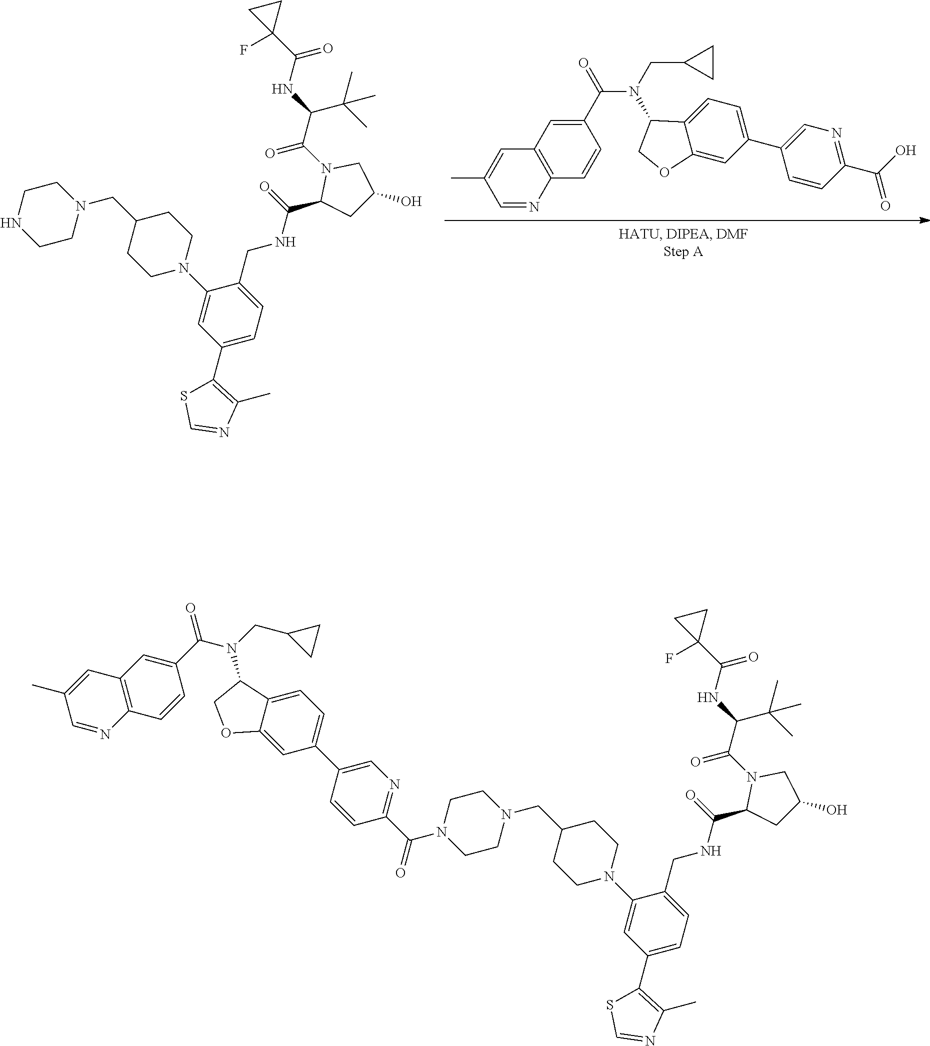 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCNCC3)CC2)c1 ; Cc1cnc2ccc(C(=O)N(CC3CC3)[C@H]3COc4cc(-c5ccc(C(=O)O)nc5)ccc43)cc2c1 ; "HATU, DIPEA, DMFStep A" ; Cc1cnc2ccc(C(=O)N(CC3CC3)[C@H]3COc4cc(-c5ccc(C(=O)N6CCN(CC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)cc2c1]

Example A144 was synthesized following the same procedures as described in Example A29 (2.2 mg, yield 6%) as a white solid. LC-MS (ESI): mass calcd, for C₆₅H₇₅FN₁₀O₇S, 1158.55; m/z found, 1159.8 [M+H]⁺. ¹H NMR (400 MHz, DMSO-d₆) δ 8.97 (s, 1H), 8.91 (s, 1H), 8.84 (d, J=2.0 Hz, 1H), 8.54-8.44 (m, 1H), 8.24-8.20 (m, 2H), 8.07-8.03 (m, 2H), 7.76 (d, J=8.6 Hz, 1H), 7.66-7.60 (m, 2H), 7.44-7.37 (m, 2H), 7.31-7.26 (m, 2H), 7.12-7.08 (m, 2H), 5.16 (s, 1H), 4.89-4.64 (m, 2H), 4.62-4.57 (m, 1H), 4.50-4.45 (m, 1H), 4.39-4.26 (m, 3H), 3.69-3.65 (m, 2H), 3.62-3.58 (m, 1H), 3.49-3.46 (m, 2H), 3.26-3.24 (m, 2H), 3.12-3.04 (m, 4H), 2.70-2.62 (m, 2H), 2.49-2.49 (m, 3H), 2.45 (s, 3H), 2.37 (s, 4H), 2.26-2.22 (m, 2H), 2.10-2.06 (m, 1H), 1.94-1.89 (m, 1H), 1.85-1.80 (m, 2H), 1.70-1.64 (m, 1H), 1.38-1.29 (m, 4H), 1.23-1.20 (m, 2H), 0.98-0.95 (m, 9H), 0.90-0.77 (m, 2H), 0.36-0.25 (m, 2H), −0.21-0.27 (m, 1H).

### Example A145. 4-amino-N-((S)-6-(6-(4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)methyl)piperazine-1-carbonyl)pyridin-3-yl)-2,3-dihydrobenzofuran-3-yl)-N-methyl-1,3-dihydrofuro[3,4-c][1,7]naphthyridine-8-carboxamide

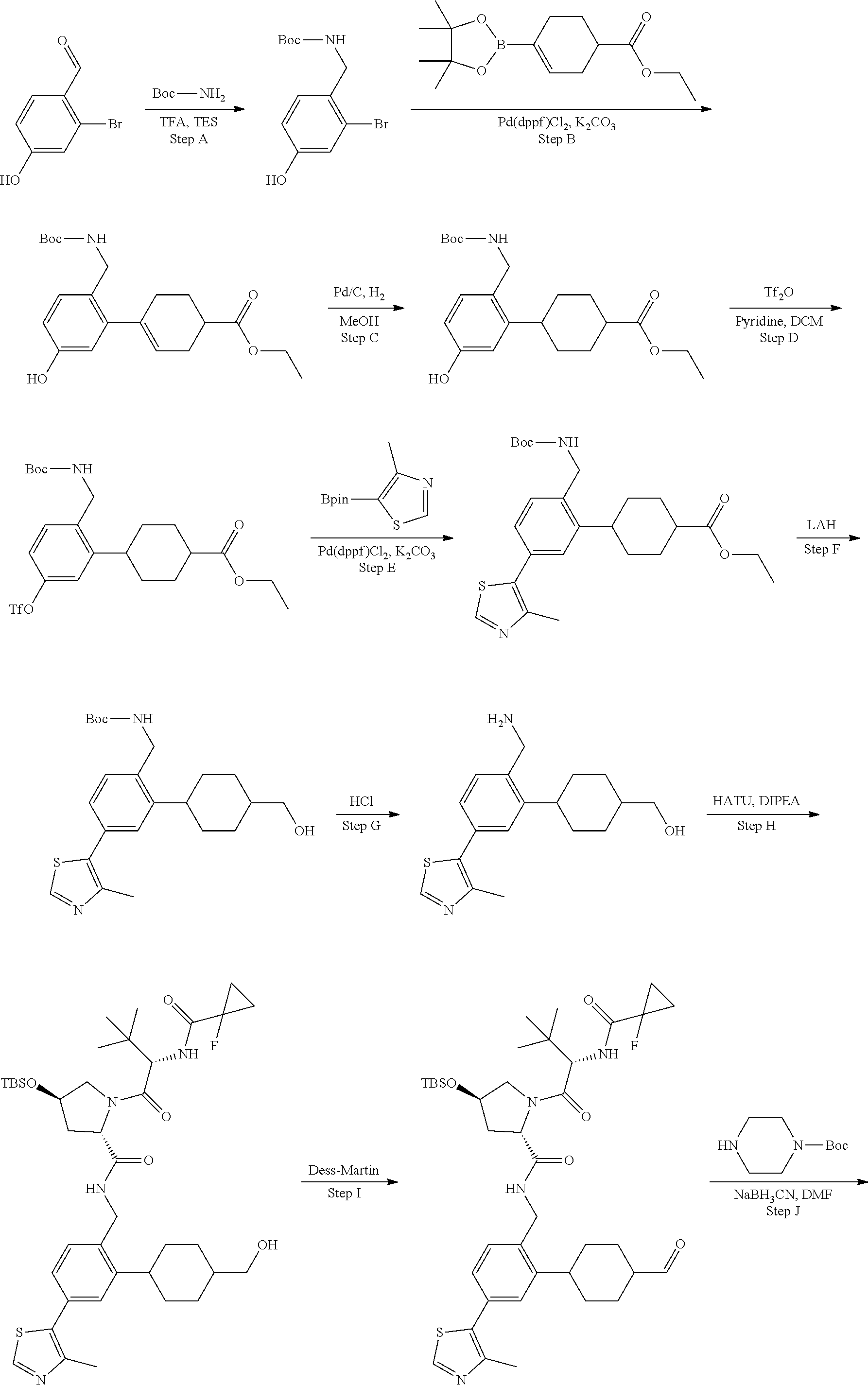

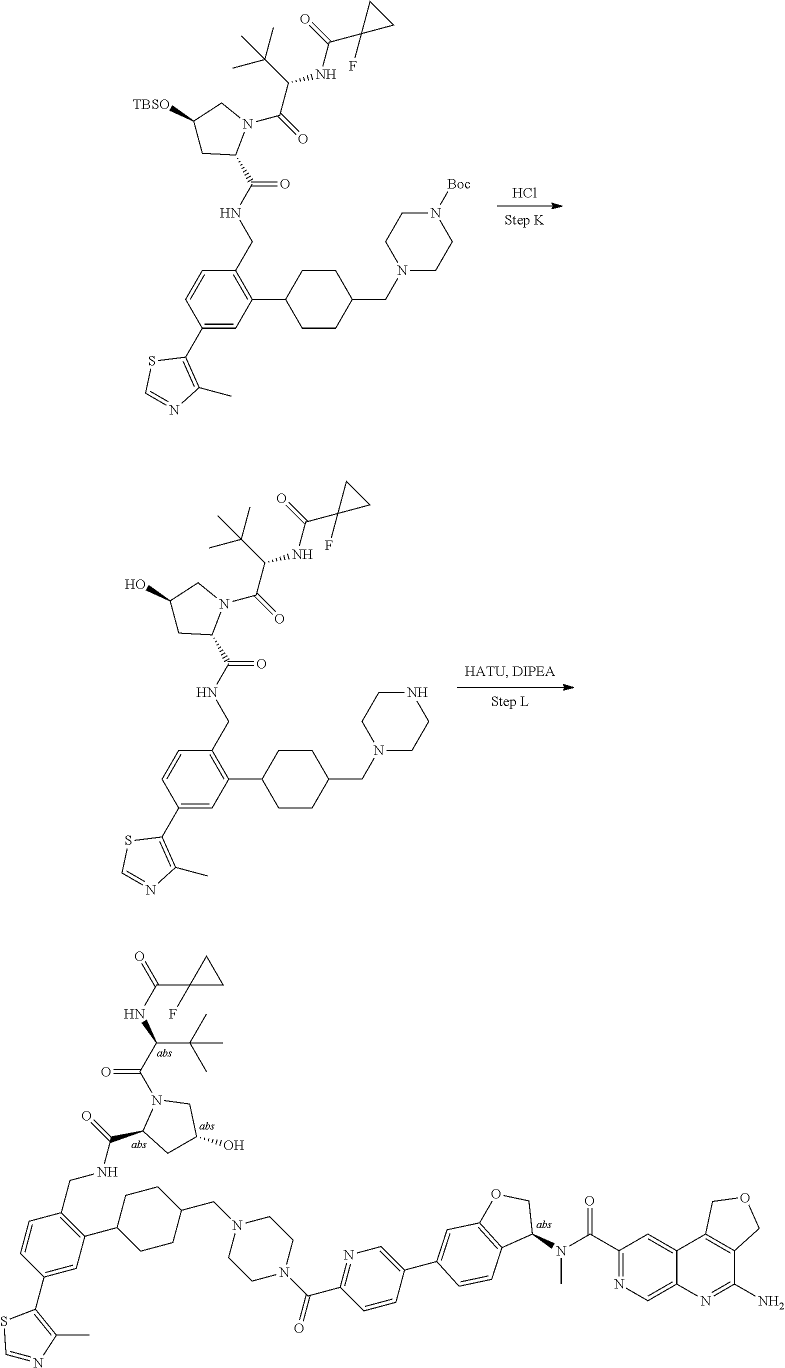

Step A: tert-butyl(2-bromo-4-hydroxybenzyl)carbamate

To a solution of 2-bromo-4-hydroxybenzaldehyde (3 g, 14.9 mmol, 1.0 eq.) and tert-butyl carbamate (5.3 g, 44.8 mmol, 3.0 eq.) in DCM (1 mL) and MeCN (3 mL) was added dropwise Et3 SiH (50 g, 44.8 mmol, 3.0 eq.) and TFA (3.4 g, 29.8 mmol, 2.0 eq.). The mixture was stirred at room temperature for 16 hours under nitrogen. After cooling to room temperature. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~20% EtOAc in PE) to afford tert-butyl(2-bromo-4-hydroxybenzyl)carbamate (4 g, 88% yield) as a yellow solid. LC-MS (ESI): mass calcd. For C₁₂H₁₆BrNO₃, 301.03; m/z found, 302 [M+H]⁺.

Step B: ethyl 2′-(((tert-butoxycarbonyl)amino)methyl)-5′-hydroxy-2,3,4,5-tetrahydro-[1,1′-biphenyl]-4-carboxylate

A mixture of tert-butyl(2-bromo-4-hydroxybenzyl)carbamate (2 g, 6.62 mmol, 1.0 eq.), ethyl 4-(4,4,5,5-tetramethyl-1,3,2-dioxaborolan-2-yl)cyclohex-3-ene-1-carboxylate (2.2 g, 7.94 mmol, 1.2 eq.), Pd (dppf) C12 (484 mg, 662 μmol, 0.1 eq.) and K₂CO₃ (2.7 g, 19.9 mmol, 3.0 eq.) in 1,4-Dioxane (30 mL) and H₂O (6 mL) was stirred at 90° C. for 3 hours under nitrogen. After cooling to room temperature. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~30% EtOAc in PE) to afford ethyl 2′-(((tert-butoxycarbonyl)amino)methyl)-5′-hydroxy-2,3,4,5-tetrahydro-[1,1′-biphenyl]-4-carboxylate (2 g, 81% yield) as a yellow solid. LC-MS (ESI): mass calcd. For C₂₁H₂₉NO₅, 375.20; m/z found, 376 [M+H]⁺.

Step C: ethyl 4-(2-(((tert-butoxycarbonyl)amino)methyl)-5-hydroxyphenyl)cyclohexane-1-carboxylate

To a solution of ethyl 2′-(((tert-butoxycarbonyl)amino)methyl)-5′-hydroxy-2,3,4,5-tetrahydro-[1,1′-biphenyl]-4-carboxylate (900 mg, 2.40 mmol, 1.0 eq.) in MeOH (5 mL) was added Pd/C (150 mg, 10% Wt) and Pd (OH) 2/C (150 mg, 10% Wt) at 35° C. for 16 hours under nitrogen. The resulting solution was filtered and removed under reduced pressure to afford ethyl 4-(2-(((tert-butoxycarbonyl)amino)methyl)-5-hydroxyphenyl)cyclohexane-1-carboxylate (900 mg, 99% yield) as a yellow solid. LC-MS (ESI): mass calcd. For C₂₁H₃₁NO₅, 377.22; m/z found, 378 [M+H]⁺.

Step D: ethyl 4-(2-(((tert-butoxycarbonyl)amino)methyl)-5-(((trifluoromethyl)sulfonyl)oxy)phenyl)cyclohexane-1-carboxylate

A mixture of ethyl 4-(2-(((tert-butoxycarbonyl)amino)methyl)-5-hydroxyphenyl)cyclohexane-1-carboxylate (900 mg, 2.3 mmol, 1.0 eq.), Pyridine (419 mg, 1.06 mmol, 2.0 eq.) and trifluoromethanesulfonic anhydride (1.3 g, 4.78 mol, 2.0 eq.) in DCM (10 mL) was stirred at room temperature for 2 hour under nitrogen. After cooling to room temperature. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~30% EtOAc in PE) to afford ethyl 4-(2-(tert-butoxycarbonyl)amino)methyl)-5-(((trifluoromethyl)sulfonyl)oxy)phenyl)cyclohexane-1-carboxylate (1.1 g, 91% yield) as a yellow solid. LC-MS (ESI): mass calcd. For C₂₂H₃₀F₃NO₇S, 509.17; m/z found, 510 [M+H]⁺.

Step E: ethyl 4-(2-(((tert-butoxycarbonyl)amino)methyl)-5-(4-methylthiazol-5-yl)phenyl)cyclohexane-1-carboxylate

To a mixture of ethyl 4-(2-(((tert-butoxycarbonyl)amino)methyl)-5-(((trifluoromethyl)sulfonyl)oxy)phenyl)cyclohexane-1-carboxylate (1.1 g, 2.2 mmol, 1.0 eq.) in 1,4-Dioxane (20 mL): Water (3 mL) was added 4-methyl-5-(4,4,5,5-tetramethyl-1,3,2-dioxaborolan-2-yl) thiazole (535 mg, 2.4 mmol, 1.1 eq.), K₂CO₃ (746 mg, 5.4 mmol, 2.5 eq.) and PdCl₂ (dppf)(158 mg, 216 μmol, 0.1 eq.). The reaction mixture was stirred at 100° C. for 3 hours under N₂. The resulting mixture was filtered and concentrated. The crude product was purified by flash chromatography (100-200 mesh silica gel, 0%~70% EA in PE) to afford ethyl 4-(2-(((tert-butoxycarbonyl)amino)methyl)-5-(4-methylthiazol-5-yl)phenyl)cyclohexane-1-carboxylate (600 mg, 60% yield) as a colorless oil. LC-MS (ESI): mass calcd. For C₂₅H₃₄N₂O₄S, 458.22; m/z found, 459 [M+H]⁺.

Step F: tert-butyl(2-(4-(hydroxymethyl)cyclohexyl)-4-(4-methylthiazol-5-yl)benzyl)carbamate

To a mixture of ethyl 4-(2-(((tert-butoxycarbonyl)amino)methyl)-5-(4-methylthiazol-5-yl)phenyl)cyclohexane-1-carboxylate (600 mg, 1.3 mmol, 1.0 eq.) in THF (10 mL) was added LAH (149 mg, 3.9 mmol, 3.0 eq.) at 0° C. The reaction mixture was stirred at −780C for 2 hours. LCMS showed the reaction was completed. The mixture was quenched with Potassium sodium tartrate, EtOAc (50 mL) was added followed by Celite, filtered and concentrated to afford the tert-butyl(2-(4-(hydroxymethyl)cyclohexyl)-4-(4-methylthiazol-5-yl)benzyl)carbamate (500 mg, 91% yield) as a white solid. LC-MS (ESI): mass calcd. For C₂₅H₃₄N₂O₄S, 458.22; m/z found, 459 [M+H]⁺.

Step G: (4-(2-(aminomethyl)-5-(4-methylthiazol-5-yl)phenyl)cyclohexyl) methanol

A mixture of tert-butyl(2-(4-(hydroxymethyl)cyclohexyl)-4-(4-methylthiazol-5-yl)benzyl)carbamate (60 mg, 144 μmol, 1.0 eq.) and HCl/dioxane (1 mL) in DCM (3 mL) was stirred at room temperature for 1 hour under nitrogen. The resulting solution was removed under reduced to afford (4-(2-(aminomethyl)-5-(4-methylthiazol-5-pressure yl)phenyl)cyclohexyl) methanol (40.0 mg, 87% yield) as a yellow solid. LC-MS (ESI): mass calcd. For C₁₈H₂₄N₂Os, 316.16; m/z found, 317 [M+H]⁺.

Step H: (2S,4R)-4-((tert-butyldimethylsilyl)oxy)-1-((S)-2-(1-fluorocyclopropane-1-carboxamido)-3,3-dimethylbutanoyl)-N-(2-(4-(hydroxymethyl)cyclohexyl)-4-(4-methylthiazol-5-yl)benzyl)pyrrolidine-2-carboxamide

A mixture of (4-(2-(aminomethyl)-5-(4-methylthiazol-5-yl)phenyl)cyclohexyl) methanol (50 mg, 158 μmol, 1.0 eq.), (2S,4R)-4-((tert-butyldimethylsilyl)oxy)-1-((S)-2-(1-fluorocyclopropane-1-carboxamido)-3,3-dimethylbutanoyl)pyrrolidine-2-carboxylic acid (77 mg, 174 μmol, 1.1 eq.), HATU (120 mg, 316 μmol, 2.0 eq.) and DIPEA (61.1 mg, 474 μmol, 3.0 eq.) in DMF (3.00 mL) was stirred at room temperature for 30 min under nitrogen. After cooling to room temperature, The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~10% MeOH in DCM) to afford (2S,4R)-4-((tert-butyldimethylsilyl)oxy)-1-((S)-2-(1-fluorocyclopropane-1-carboxamido)-3,3-dimethylbutanoyl)-N-(2-(4-(hydroxymethyl)cyclohexyl)-4-(4-methylthiazol-5-yl)benzyl)pyrrolidine-2-carboxamide (50 mg, 41% yield) as a yellow solid. LC-MS (ESI): mass calcd. For C₃₉H₅₉FN₄O₅SSi, 742.40; m/z found, 743 [M+H]⁺.

Step I: (2S,4R)-4-((tert-butyldimethylsilyl)oxy)-1-((S)-2-(1-fluorocyclopropane-1-carboxamido)-3,3-dimethylbutanoyl)-N-(2-(4-formylcyclohexyl)-4-(4-methylthiazol-5-yl)benzyl)pyrrolidine-2-carboxamide

A mixture of (2S,4R)-4-((tert-butyldimethylsilyl)oxy)-1-((S)-2-(1-fluorocyclopropane-1-carboxamido)-3,3-dimethylbutanoyl)-N-(2-(4-(hydroxymethyl)cyclohexyl)-4-(4-methylthiazol-5-yl)benzyl)pyrrolidine-2-carboxamide (50 mg 67.3 μmol, 1.0 eq.) and Dess-Martin (57.1 mg, 135 μmol 2.0 eq.) in DCM (3 mL) was stirred at room temperature for 2 hours under nitrogen. After cooling to room temperature. The resulting solution was diluted with 30 mL of water and extracted with EtOAc (20 mL×3). The combined organic layers were washed with brine (50 mL), dried over Na₂SO₄ and concentrated under reduced pressure. The crude product was purified by flash column chromatography on silica gel (100-200 mesh silica gel, 0~10% MeOH in DCM) to afford (2S,4R)-4-((tert-butyldimethylsilyl)oxy)-1-((S)-2-(1-fluorocyclopropane-1-carboxamido)-3,3-dimethylbutanoyl)-N-(2-(4-formylcyclohexyl)-4-(4-methylthiazol-5-yl)benzyl)pyrrolidine-2-carboxamide (40 mg, 80% yield) as a white solid. LC-MS (ESI): mass calcd. For C₃₉H₅₇FN₄O₅SSi, 740.38; m/z found, 741 [M+H]⁺.

Step J: tert-butyl 4-((4-(2-(((2S,4R)-4-((tert-butyldimethylsilyl)oxy)-1-((S)-2-(1-fluorocyclopropane-1-carboxamido)-3,3-dimethylbutanoyl)pyrrolidine-2-carboxamido)methyl)-5-(4-methylthiazol-5-yl)phenyl)cyclohexyl)methyl)piperazine-1-carboxylate

A mixture of (2S,4R)-4-((tert-butyldimethylsilyl)oxy)-1-((S)-2-(1-fluorocyclopropane-1-carboxamido)-3,3-dimethylbutanoyl)-N-(2-(4-formylcyclohexyl)-4-(4-methylthiazol-5-yl)benzyl)pyrrolidine-2-carboxamide (25.0 mg, 33.7 μmol, 1.0 eq.), AcOH (1.01 mg, 16.9 μmol, 0.5 eq.) and tert-butyl piperazine-1-carboxylate (13 mg, 67.5 μmol, 2.0 eq.) in MeOH (5 mL) was stirred at room temperature for 1 hour under nitrogen. Then NaBH₃CN (4.25 mg, 67.5 μmol, 2.0 eq.) was added to this mixture and stirred at room temperature for 1 hour. After cooling to room temperature, The resulting solution was diluted with 10 mL of water and extracted with EtOAc (10 mL×3). The combined organic layers were washed with brine (20 mL), dried over Na₂SO₄ and concentrated under reduced pressure. The crude product was purified by flash column chromatography on silica gel (100-200 mesh silica gel, 0~10% MeOH in DCM) to afford tert-butyl 4-((4-(2-(((2S,4R)-4-((tert-butyldimethylsilyl)oxy)-1-((S)-2-(1-fluorocyclopropane-1-carboxamido)-3,3-dimethylbutanoyl)pyrrolidine-2-carboxamido)methyl)-5-(4-methylthiazol-5-yl)phenyl)cyclohexyl)methyl)piperazine-1-carboxylate (30.0 mg, 97% yield) as a yellow solid.

LC-MS (ESI): mass calcd. For C₄₈H₇₅FN₆O₆SSi, 910.52; m/z found, 911 [M+H]⁺.

Steps K-L: 4-amino-N-(S)-6-(6-(4-(4-(2-(28,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)methyl)piperazine-1-carbonyl)pyridin-3-yl)-2,3-dihydrobenzofuran-3-yl)-N-methyl-1,3-dihydrofuro[3,4-c][1,7]naphthyridine-8-carboxamide

Example 145 was synthesized following the same procedures as described in Example A23 (2.2 mg, 7% yield over 2 steps) as a white solid. LC-MS (ESI): mass calcd. for C₆₃H₇₂FN₁₁O₈S, 1161.53; m/z found, 1162 [M+H]⁺. ¹H NMR (400 MHz, DMSO-d₆) δ 8.99 (s, 1H), 8.92 (s, 2H), 8.48 (s, 1H), 8.24 (s, 1H), 7.88-7.82 (m, 1H), 7.67-7.61 (m, 1H), 7.42 (d, J=8.1 Hz, 3H), 7.27 (dt, J=13.4, 9.2 Hz, 4H), 7.10-7.04 (m, 2H), 5.39 (s, 2H), 5.16 (d, J=3.5 Hz, 1H), 5.06 (s, 2H), 4.71-4.63 (m, 2H), 4.59 (d, J=9.3 Hz, 1H), 4.50-4.45 (m, 1H), 4.40-4.30 (m, 3H), 3.68-3.57 (m, 4H), 3.50-3.42 (m, 3H), 3.37-3.34 (m, 2H), 3.31-3.29 (m, 2H), 2.79-2.71 (m, 5H), 2.45 (s, 3H), 2.23-2.13 (m, 1H), 2.08-2.03 (m, 1H), 1.93-1.85 (m, 2H), 1.81-1.74 (m, 2H), 1.69-1.45 (m, 6H), 1.40-1.32 (m, 2H), 1.23-1.20 (m, 2H), 0.98 (s, 9H).

### Example A146. 2-amino-N-((S)-6-(6-(4-(2-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)ethyl)piperazine-1-carbonyl)pyridin-3-yl)-2,3-dihydrobenzofuran-3-yl)-N,3-dimethylquinoline-6-carboxamide

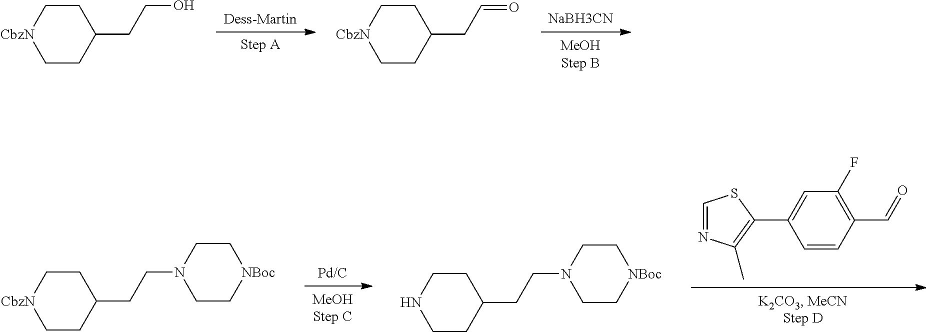

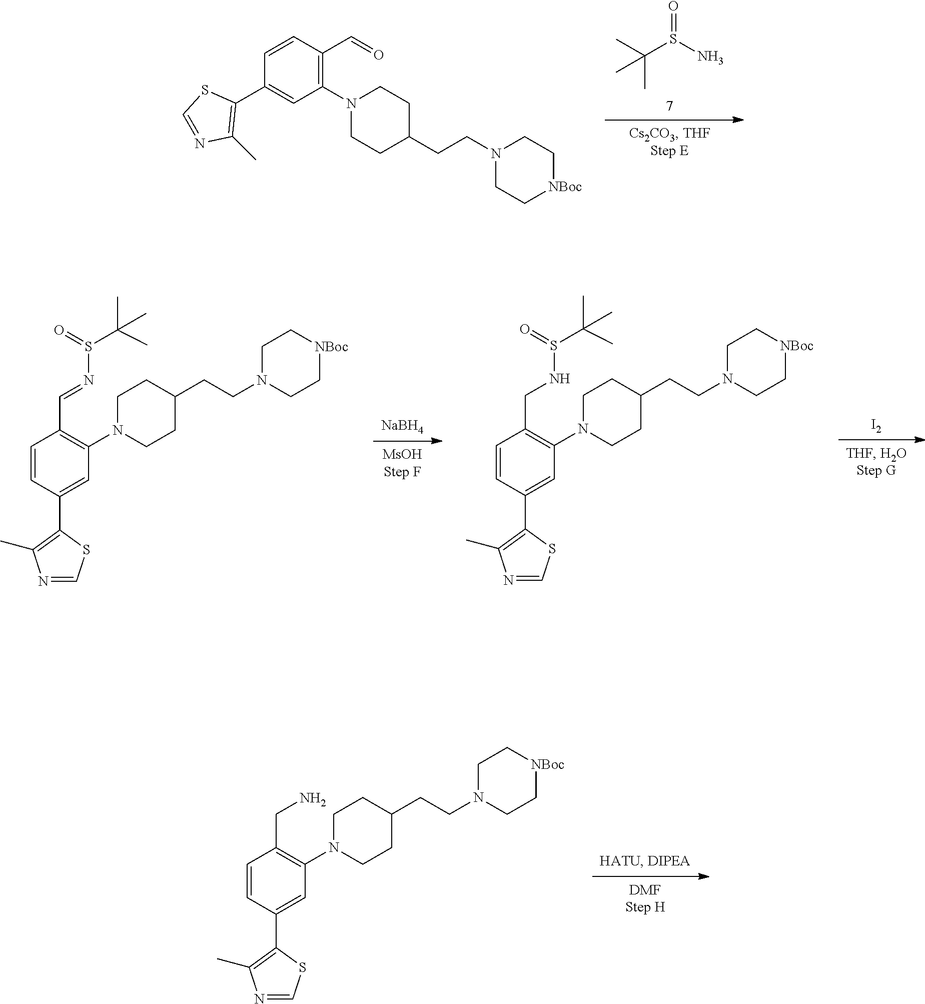

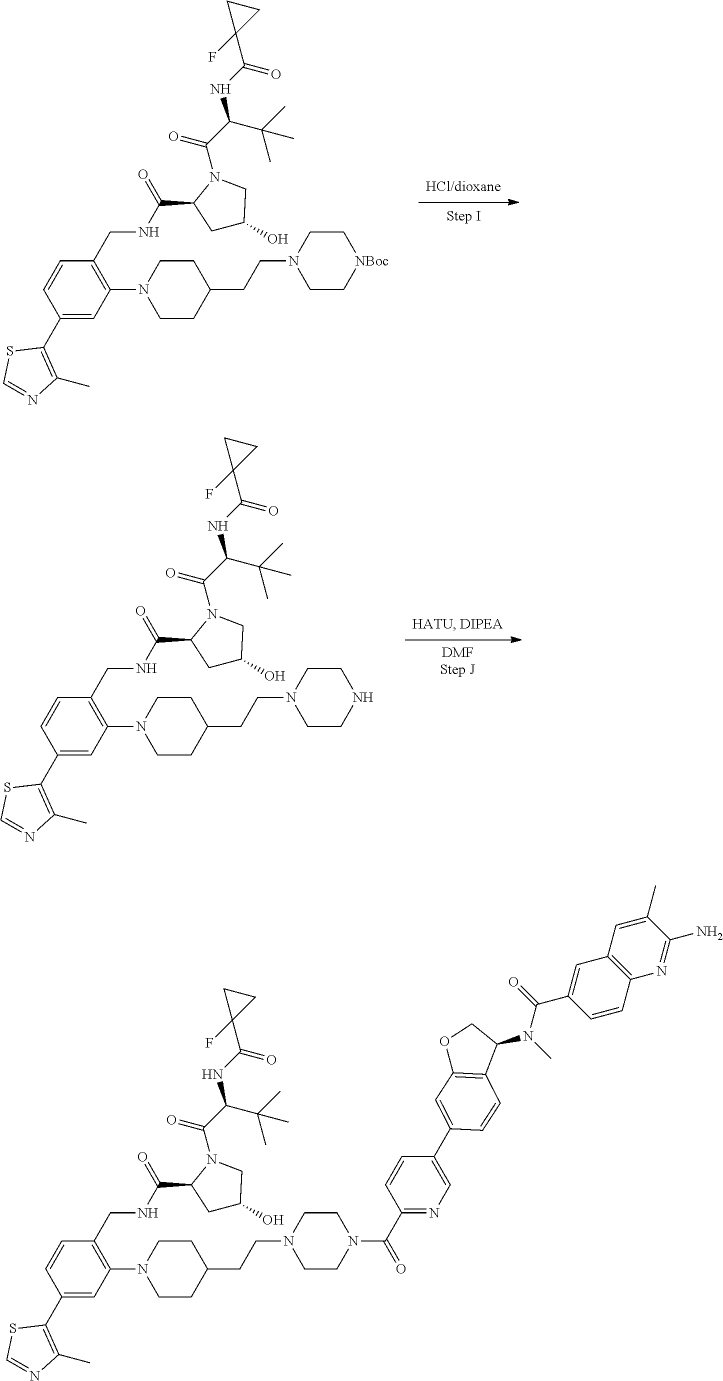 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CCN3CCN(C(=O)OC(C)(C)C)CC3)CC2)c1 ; "Step I" ; Cc1ncsc1-c1ccc(CNC(=O)[C@@H]2C[C@@H](O)CN2C(=O)[C@@H](NC(=O)C2(F)CC2)C(C)(C)C)c(N2CCC(CCN3CCNCC3)CC2)c1 ; "DMFStep J" ; Cc1cc2cc(C(=O)N(C)[C@@H]3COc4cc(-c5ccc(C(=O)N6CCN(CCC7CCN(c8cc(-c9scnc9C)ccc8CNC(=O)[C@@H]8C[C@@H](O)CN8C(=O)[C@@H](NC(=O)C8(F)CC8)C(C)(C)C)CC7)CC6)nc5)ccc43)ccc2nc1N]

Step A: benzyl 4-(2-oxoethyl)piperidine-1-carboxylate

To a solution of benzyl 4-(2-hydroxyethyl)piperidine-1-carboxylate (2 g, 7.59 mmol, 1 eq.) in DCM (30 mL) was added Dess-Martin (3.5 g, 8.35 mmol, 1.1 eq.) at room temperature. The mixture was stirred at 40° C. for 1 hour under nitrogen. After cooling to room temperature, the resulting solution was diluted with 80 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~20% EtOAc in PE) to afford benzyl 4-(2-oxoethyl)piperidine-1-carboxylate (1.4 g, 70% yield) as a colorless oil. LC-MS (ESI): mass calcd. For C₁₅H₁₉NO₃, 261.14; m/z found, 262 [M+H]⁺.

Step B: tert-butyl 4-(2-(1-((benzyloxy)carbonyl)piperidin-4-yl)ethyl)piperazine-1-carboxylate

To a solution of benzyl 4-(2-oxoethyl)piperidine-1-carboxylate (700 mg, 2.68 mmol, 1.0 eq.), tert-butyl piperazine-1-carboxylate (748 mg, 4.02 mmol, 1.5 eq.), tert-butyl piperazine-1-carboxylate (748 mg, 4.02 mmol, 1.5 eq.), AcOH (80 mg, 1.34 mmol, 0.5 eq.) and tert-butyl piperazine-1-carboxylate (748 mg, 4.02 mmol, 1.5 eq.) in DCE (10 mL) was stirred at room temperature for 2 hours under nitrogen. Then NaBH (OAc) 3 (1.14 g, 5.36 mmol, 2.0 eq.) was added to this mixture and stirred at room temperature for 2 hours. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~30% EtOAc in PE) to afford tert-butyl 4-(2-(1-((benzyloxy)carbonyl)piperidin-4-yl)ethyl)piperazine-1-carboxylate (800 mg, 69% yield) as a yellow solid. LC-MS (ESI): mass calcd. For C₂₄H₃₇N₃O₄, 431.28; m/z found, 432 [M+H]⁺.

Step C: tert-butyl 4-(2-(piperidin-4-yl)ethyl)piperazine-1-carboxylate

To a solution of tert-butyl 4-(2-(1-((benzyloxy)carbonyl)piperidin-4-yl)ethyl)piperazine-1-carboxylate (400 mg, 927 μmol, 1.0 eq.) in MeOH (3 mL) was added Pd/C (150 mg) at room temperature. The mixture was stirred at room temperature for 16 hours under nitrogen. The resulting solution was filtered and removed under reduced pressure to afford tert-butyl 4-(2-(piperidin-4-yl)ethyl)piperazine-1-carboxylate (260 mg, 94% yield) as a colorless oil. LC-MS (ESI): mass calcd. For C₁₆H₃₁N₃O₂, 297.24; m/z found, 298 [M+H]⁺.

Step D-J: 2-amino-N-(S)-6-16-(4-(2-(1-(2-(2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)ethyl)piperazine-1-carbonyl)pyridin-3-yl)-2,3-dihydrobenzofuran-3-yl)-N,3-dimethylquinoline-6-carboxamide

Example A146 was synthesized following the same procedures as described in Example A126 (2.6 mg, 4% yield over 7 steps) as a white solid. LC-MS (ESI): mass calcd. for C₆₃H₇₄FN₁₁O₇S, 1147.55; m/z found, 1148.5 [M+H]⁺. ¹H NMR (400 MHz, DMSO-d₆) δ 8.98 (s, 1H), 8.92 (s, 1H), 8.57-8.42 (m, 1H), 8.27-8.21 (m, 1H), 8.13 (s, 1H), 7.90 (s, 1H), 7.83 (s, 1H), 7.75-7.68 (m, 1H), 7.63-7.59 (m, 1H), 7.54 (d, J=7.7 Hz, 2H), 7.44-7.37 (m, 2H), 7.34 (s, 1H), 7.29-7.25 (m, 1H), 7.13-7.08 (m, 2H), 5.17 (d, J=3.5 Hz, 1H), 4.79-4.67 (m, 2H), 4.62-4.57 (m, 1H), 4.51-4.46 (m, 1H), 4.40-4.30 (m, 3H), 3.67-3.60 (m, 2H), 3.54-3.35 (m, 5H), 3.28-3.22 (m, 2H), 3.13-2.96 (m, 4H), 2.72-2.62 (m, 7H), 2.45 (s, 3H), 2.24 (s, 3H), 2.12-2.06 (m, 1H), 1.95-1.88 (m, 1H), 1.81-1.75 (m, 2H), 1.60-1.55 (m, 1H), 1.48-1.32 (m, 6H), 1.24-1.20 (m, 2H), 0.98 (s, 9H).

### Example A147. 2-amino-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)benzyl)carbamoyl)pyridin-3-yl)-2,3-dihydrobenzofuran-3-yl)-N,3-dimethylquinoline-6-carboxamide

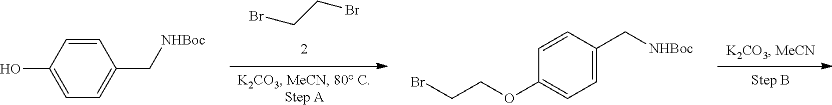

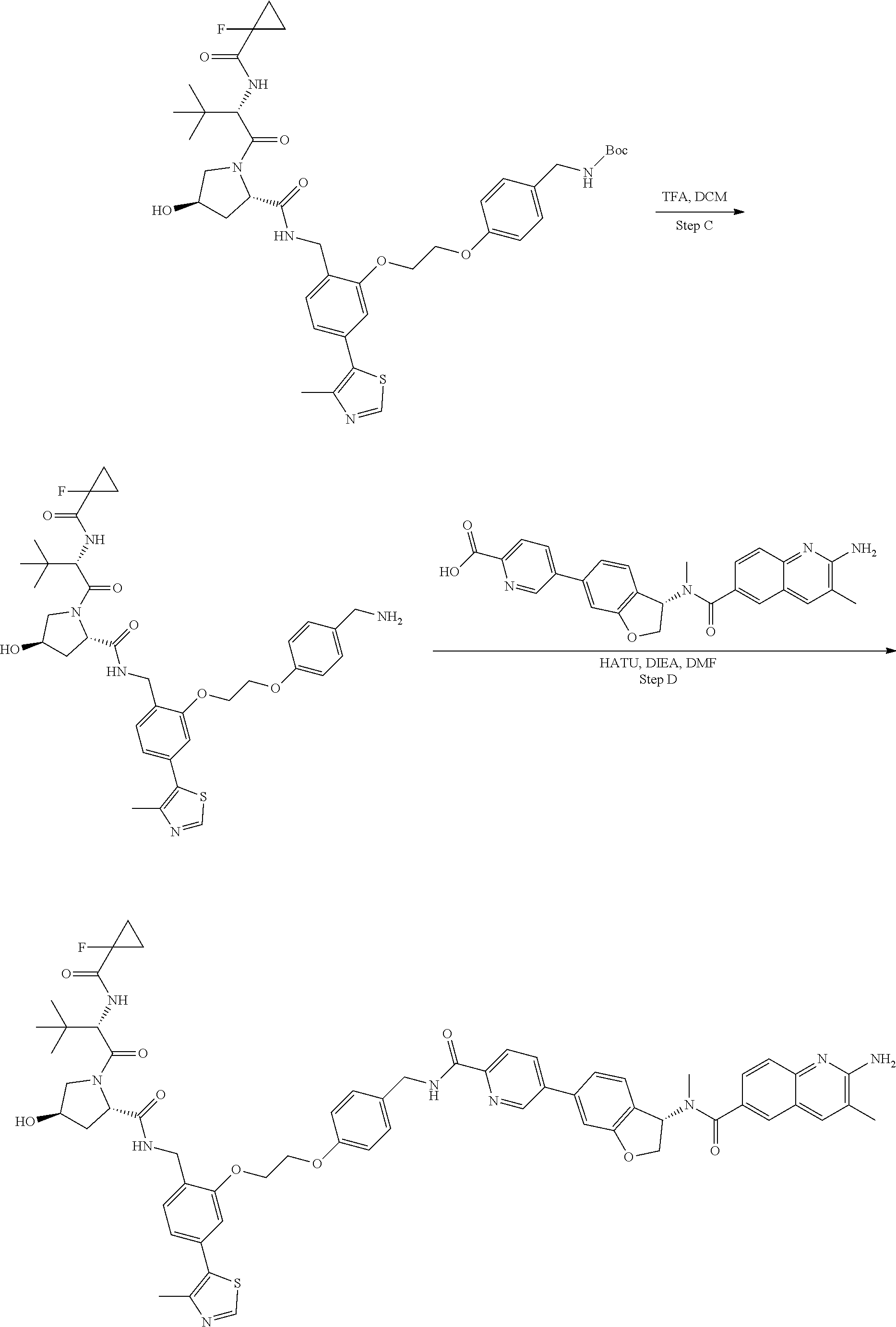

Example A147 was synthesized following the same procedures as described in Example A29 (1.4 mg, 7% yield over 4 steps) as a white solid. LC-MS (ESI): mass calcd. for C₆₁H₆₄FNO₉S, 1117.45; m/z found, 1118.4 [M+H]⁺. ¹H NMR (400 MHz, DMSO-d₆) δ 9.33 (t, J=6.4 Hz, 1H), 9.05 (s, 1H), 9.01 (s, 1H), 8.70 (s, 2H), 8.54 (t, J=5.9 Hz, 1H), 8.38 (s, 1H), 8.36-8.33 (m, 1H), 8.17 (d, J=8.2 Hz, 1H), 8.11 (s, 1H), 7.93 (d, J=9.9 Hz, 1H), 7.86-7.80 (m, 1H), 7.63 (d, J=7.7 Hz, 1H), 7.47 (d, J=7.8 Hz, 2H), 7.44-7.40 (m, 1H), 7.36-7.32 (m, 3H), 7.16 (s, 1H), 7.06-7.01 (m, 3H), 4.91-4.73 (m, 2H), 4.64 (d, J=9.2 Hz, 1H), 4.59-4.54 (m, 1H), 4.53-4.49 (m, 2H), 4.47-4.44 (m, 2H), 4.41-4.37 (m, 3H), 4.34-4.24 (m, 2H), 3.70-3.64 (m, 3H), 2.70 (s, 3H), 2.52 (s, 3H), 2.40 (s, 3H), 2.14-2.08 (m, 1H), 1.98-1.92 (m, 1H), 1.45-1.38 (m, 2H), 1.30-1.27 (m, 2H), 0.99 (s, 9H).

### Example A148. N-(cyclopropylmethyl)-N-((S)-6-(6-(((1S,4S)-4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)(methyl)amino)cyclohexyl) carbamoyl)pyridin-3-yl)-2,3-dihydrobenzofuran-3-yl)-3-methylquinoline-6-carboxamide

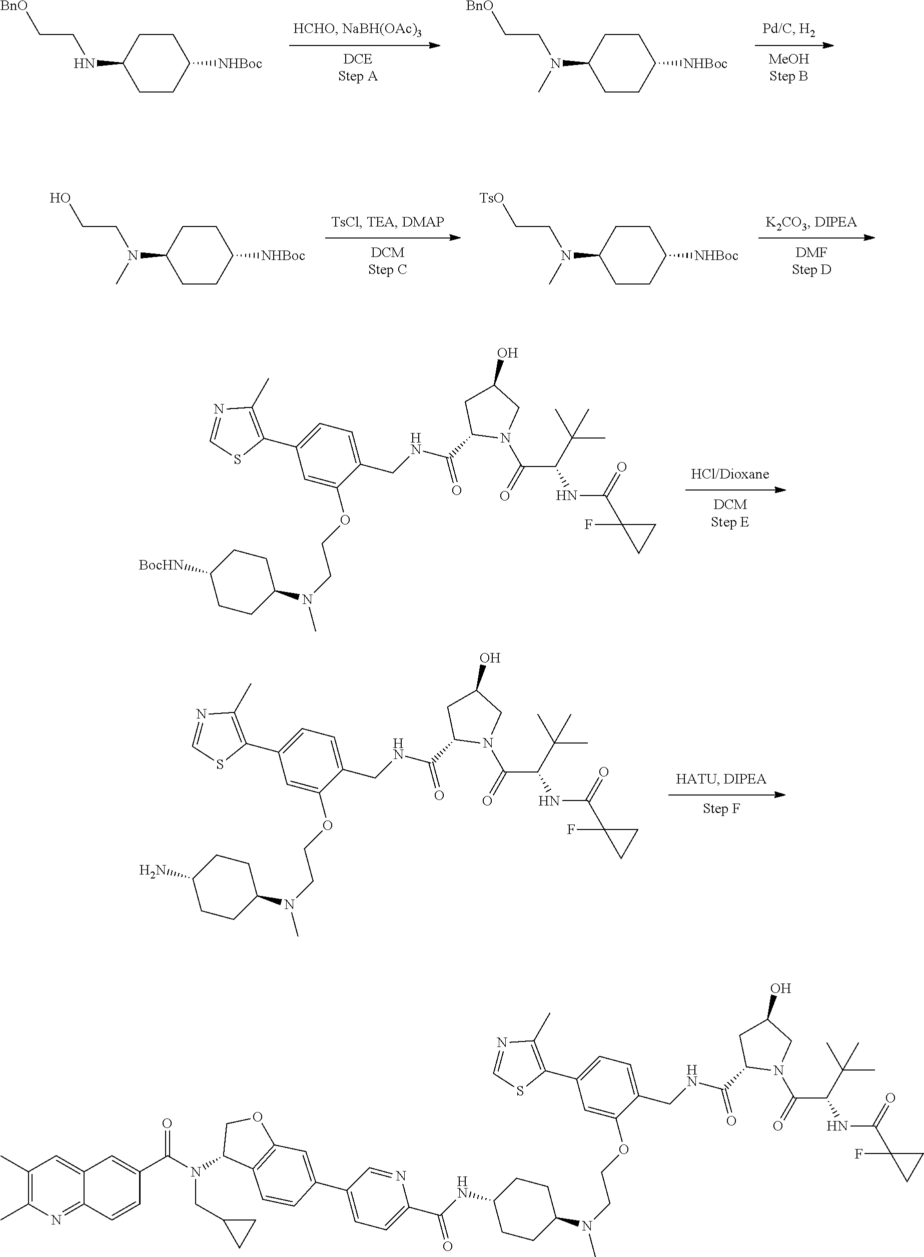

Example A148 was synthesized following the same procedures as described in Example A130 (8 mg, 3% yield over 6 steps) as a white solid. LC-MS (ESI): mass calcd, for C₆₄H₇₄FN₉O₈S, 1147.54; m/z found, 1148.5 [M+H]⁺. ¹H NMR (400 MHz, DMSO-d₆) δ 8.99 (s, 1H), 8.92 (s, 1H), 8.84 (d, J=1.8 Hz, 1H), 8.49 (d, J=6.8 Hz, 2H), 8.28-8.21 (m, 2H), 8.14 (s, 1H), 8.09-8.03 (m, 3H), 7.76 (d, J=8.0 Hz, 1H), 7.62 (d, J=7.8 Hz, 1H), 7.42-7.35 (m, 2H), 7.32-7.25 (m, 2H), 7.05 (s, 1H), 6.99-6.94 (m, 1H), 5.17 (s, 1H), 4.87-4.72 (m, 2H), 4.62-4.58 (m, 1H), 4.54-4.49 (m, 1H), 4.37-4.34 (m, 1H), 4.32-4.24 (m, 2H), 4.18 (s, 2H), 3.82-3.76 (m, 1H), 3.66-3.59 (m, 2H), 3.21-3.14 (m, 2H), 3.11-2.95 (m, 4H), 2.51 (s, 3H), 2.49-2.49 (m, 3H), 2.47 (s, 3H), 2.11-2.06 (m, 1H), 1.95-1.87 (m, 5H), 1.54-1.44 (m, 4H), 1.39-1.32 (m, 2H), 1.23-1.19 (m, 2H), 0.95 (s, 9H), 0.93-0.78 (m, 2H), 0.36-0.25 (m, 2H), −0.21 (s, 1H).

### Example A149. N1-(6-amino-5-fluoropyridin-3-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) ethoxy)ethyl)carbamoyl)pyridin-3-yl)-2,3-dihydrobenzofuran-3-yl)-N2-methyloxalamide

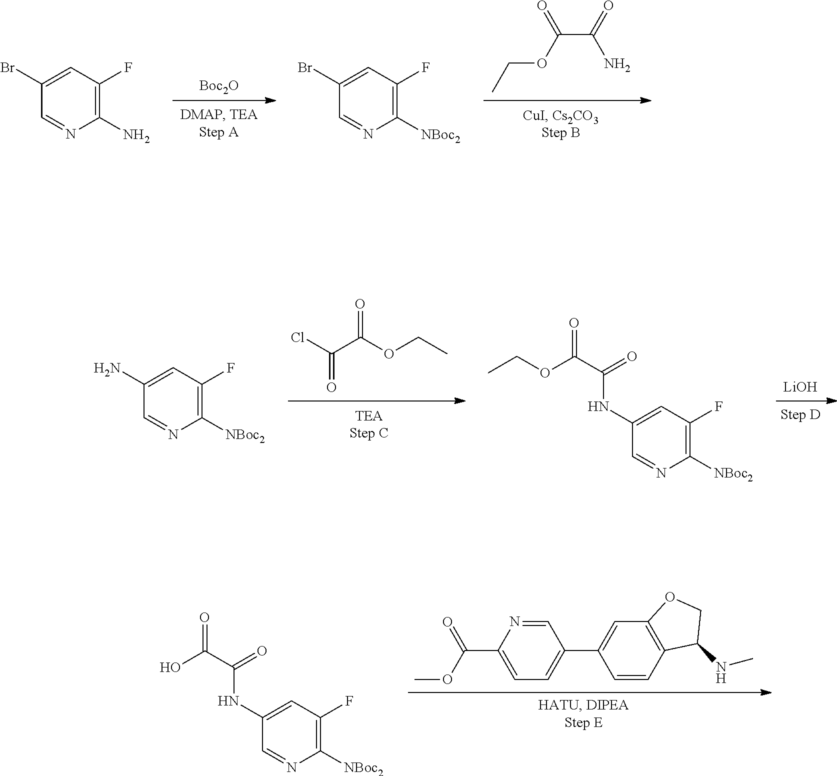

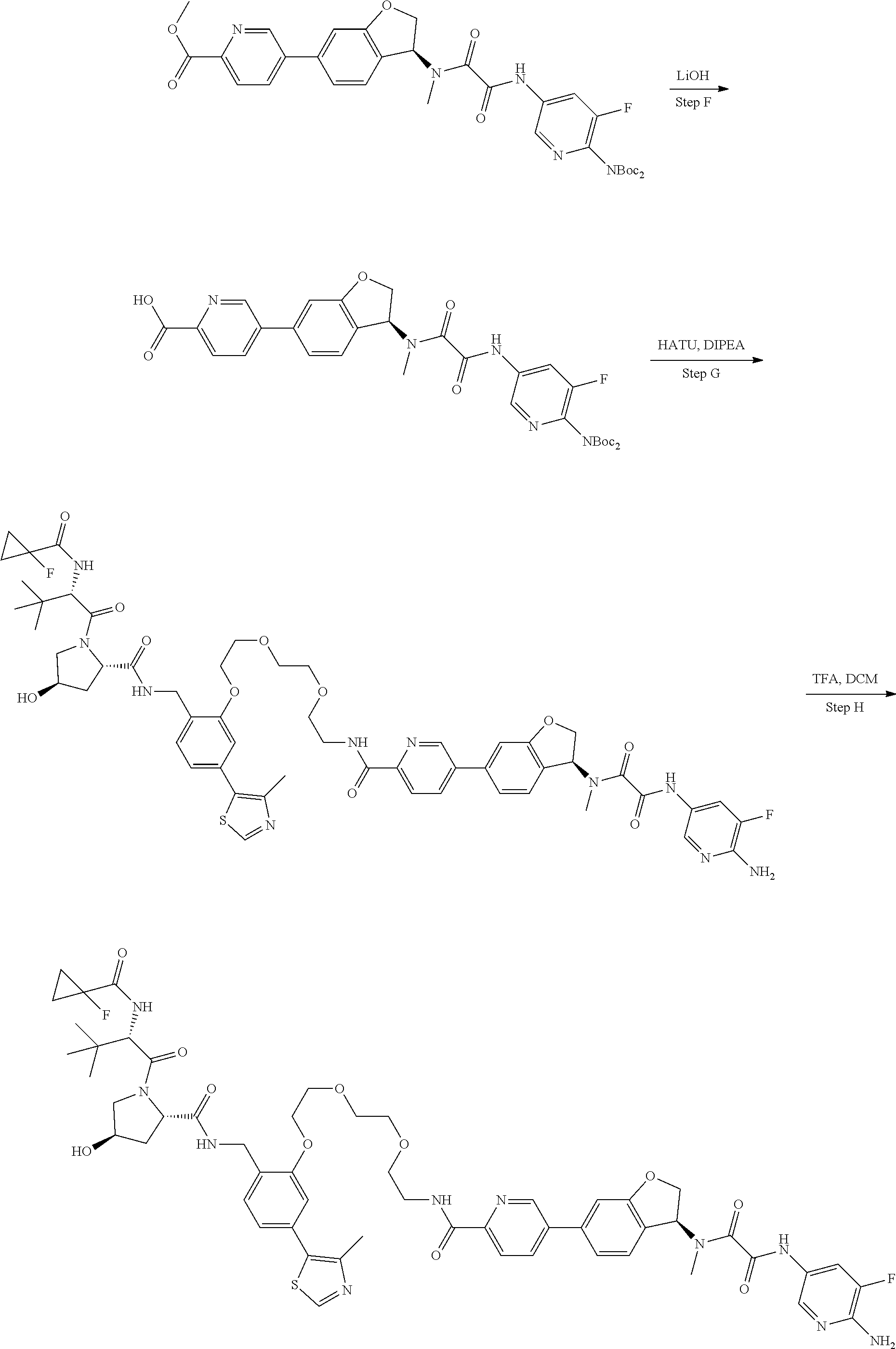

Step A: tert-butyl (5-bromo-3-fluoropyridin-2-yl)(tert-butoxycarbonyl)carbamate

To a solution of 5-bromo-3-fluoropyridin-2-amine (5 g, 26.2 mmol, 1.0 eq.) in DCM (50 mL) was added Di-tert-butyl dicarbonate (14.3 g, 65.4 mmol, 2.5 eq.), TEA (10.6 g, 105 mmol, 4.0 eq.) and DMAP (640 mg, 5.24 mmol, 0.2 eq.) at room temperature. The mixture was then stirred at room temperature for 2 hours under N₂ atmosphere. The resulting solution was diluted with 100 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~10% EtOAc in PE) to afford tert-butyl (5-bromo-3-fluoropyridin-2-yl)(tert-butoxycarbonyl)carbamate (9.7 g, 94% yield) as a white solid. LC-MS (ESI): mass calcd. For C₁₅H₂₀BrFN₂O₄, 390.1; m/z found, 391.0

Step B: tert-butyl (5-amino-3-fluoropyridin-2-yl)(tert-butoxycarbonyl)carbamate

To a solution of tert-butyl (5-bromo-3-fluoropyridin-2-yl)(tert-butoxycarbonyl)carbamate (1 g, 2.56 mmol, 1.0 eq.) in 1,4-Dioxane (10 mL) was added ethyl 2-amino-2-oxoacetate (449 mg, 3.83 mmol, 1.5 eq.), CuI (49 mg, 256 μmol, 0.1 eq.), Cs₂CO₃ (1.67 g, 2.0 eq, 5.1 mmol) and Methyl [2-(methylamino)ethyl]amine (68 mg, 767 μmol, 0.3 eq.) at room temperature. The mixture was then stirred at 100° C. for 4 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~50% EtOAc in PE) to afford tert-butyl (5-amino-3-fluoropyridin-2-yl)(tert-butoxycarbonyl)carbamate (60 mg, 7% yield) as a yellow oil. LC-MS (ESI): mass calcd. For C₁₅H₂₂FN₃O₄, 327.2; m/z found, 328.1 [M+H]⁺.

Step C: ethyl 2-((6-(bis(tert-butoxycarbonyl)amino)-5-fluoropyridin-3-yl)amino)-2-oxoacetate

To a solution of tert-butyl (5-amino-3-fluoropyridin-2-yl)(tert-butoxycarbonyl)carbamate (60 mg, 183 μmol, 1.0 eq.) in DCM (5 mL) was added ethyl 2-chloro-2-oxoacetate (28 mg, 202 μmol, 1.1 eq.) and TEA (37 mg, 367 μmol, 2.0 eq.) at 0° C. The mixture was then stirred at room temperature for 2 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~30% EtOAc in PE) to afford ethyl 2-((6-(bis(tert-butoxycarbonyl)amino)-5-fluoropyridin-3-yl)amino)-2-oxoacetate (25 mg, 32% yield) as a colourless oil. LC-MS (ESI): mass calcd. For C₁₉H₂₆FN₃O₇, 427.2; m/z found, 428.1 [M+H]⁺.

Step D: 2-((6-(bis(tert-butoxycarbonyl)amino)-5-fluoropyridin-3-yl)amino)-2-oxoacetic acid

To a solution of ethyl 2-((6-(bis(tert-butoxycarbonyl)amino)-5-fluoropyridin-3-yl)amino)-2-oxoacetate (25 mg, 58.5 μmol, 1.0 eq.) and LiOH (2 mg, 64.3 μmol, 1.1 eq.) in EtOH (2 mL) and H₂O (2 mL) was added at 0° C. The mixture was then stirred at room temperature for 1 hour under N₂ atmosphere. The resulting solution was diluted with 1 N HCl, then extracted with EtOAc (30 mL×2) and washed with brine (30 mL). The combined organic larer was dried over anhydrous sodium sulfate. The residue was concentrated under reduced pressure to afford 2-((6-(bis(tert-butoxycarbonyl)amino)-5-fluoropyridin-3-yl)amino)-2-oxoacetic acid (23 mg, 98% yield) as a yellow oil. LC-MS (ESI): mass calcd. For C₁₇H₂₂FN₃O₇, 399.1; m/z found, 400.0 [M+H]⁺.

Steps E-H: N1-(6-amino-5-fluoropyridin-3-yl)-N2-(S)-6-16-(2-(2-(2-(2-(28,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N2-methyloxalamide

Example A149 was synthesized following the same procedures as described in Example A23 (11.2 mg, 1% yield over 3 steps) as a white solid. LC-MS (ESI): mass calcd. for C₅₄H₆₂F₂N₁₀O₁₁S, 1096.4/z found, 1097.9 [M+H]⁺. ¹H NMR (400 MHz, DMSO-d₆) δ 10.80 (d, J=53.9 Hz, 1H), 8.95 (s, 1H), 8.92 (d, J=1.7 Hz, 1H), 8.75-8.67 (m, 1H), 8.54-8.42 (m, 1H), 8.26 (d, J=8.2 Hz, 1H), 8.12-8.03 (m, 2H), 7.80-7.68 (m, 1H), 7.58-7.42 (m, 1H), 7.41-7.36 (m, 2H), 7.34 (s, 1H), 7.28 (d, J=7.0 Hz, 1H), 7.01 (s, 1H), 6.95 (d, J=7.8 Hz, 1H), 6.29-6.23 (m, 1H), 6.15 (s, 2H), 5.95-5.89 (m, 1H), 5.16 (d, J=3.5 Hz, 1H), 4.82-4.70 (m, 1H), 4.69-4.61 (m, 1H), 4.59 (d, J=9.1 Hz, 1H), 4.55-4.48 (m, 1H), 4.38-4.20 (m, 3H), 4.19-4.15 (m, 2H), 3.83-3.78 (m, 2H), 3.68-3.62 (m, 3H), 3.62-3.56 (m, 5H), 3.52-3.47 (m, 2H), 2.75 (s, 1H), 2.61 (s, 2H), 2.43 (s, 3H), 2.13-2.04 (m, 1H), 1.97-1.87 (m, 1H), 1.42-1.31 (m, 2H), 1.24-1.18 (m, 2H), 0.95 (s, 9H).

### Example A150. 2-amino-N-((S)-6-(6-((2-(2-((3-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl) prop-2-yn-1-yl)oxy)ethoxy)ethyl)carbamoyl)pyridin-3-yl)-2,3-dihydrobenzofuran-3-yl)-N,3-dimethylquinoline-6-carboxamide

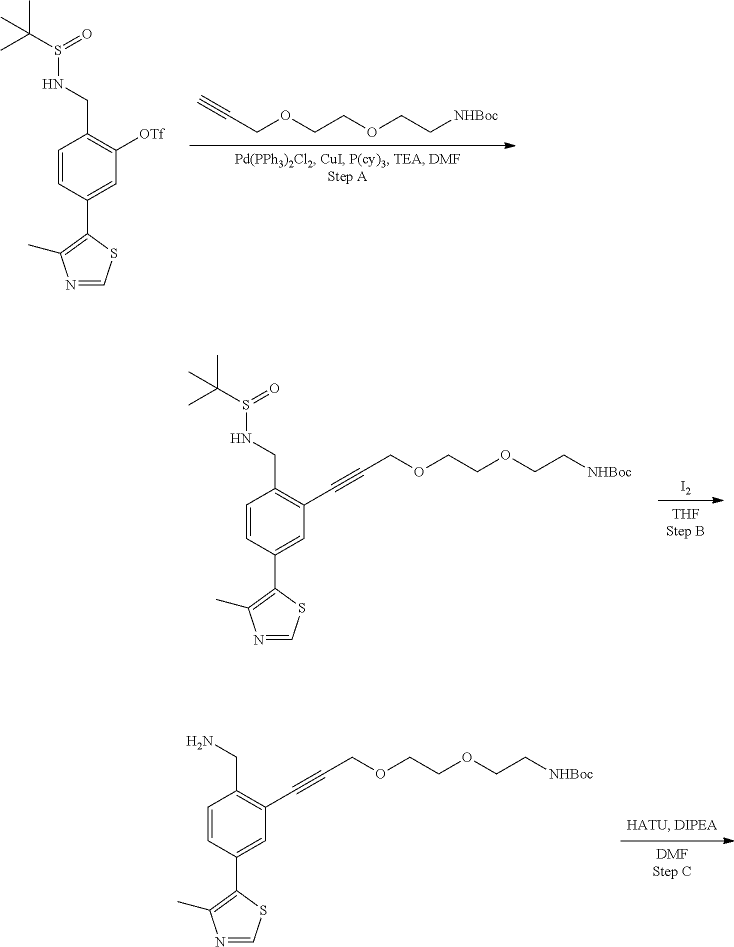

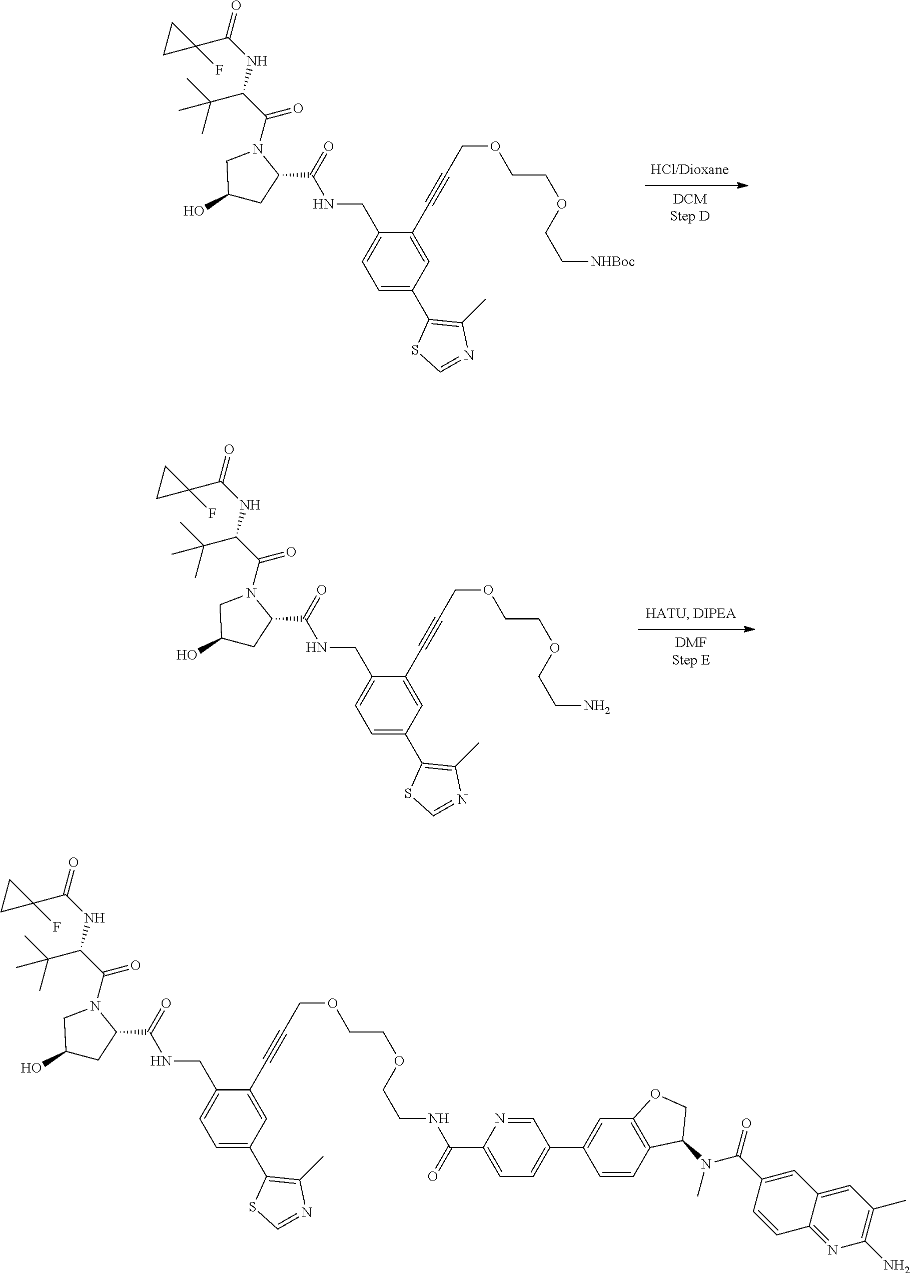 [SMILES: Cc1ncsc1-c1ccc(CNC(=O)[C@@H]2C[C@@H](O)CN2C(=O)[C@@H](NC(=O)C2(F)CC2)C(C)(C)C)c(C#CCOCCOCCNC(=O)OC(C)(C)C)c1 ; "DCMStep D" ; Cc1ncsc1-c1ccc(CNC(=O)[C@@H]2C[C@@H](O)CN2C(=O)[C@@H](NC(=O)C2(F)CC2)C(C)(C)C)c(C#CCOCCOCCN)c1 ; "DMFStep E" ; Cc1cc2cc(C(=O)N(C)[C@@H]3COc4cc(-c5ccc(C(=O)NCCOCCOCC#Cc6cc(-c7scnc7C)ccc6CNC(=O)[C@@H]6C[C@@H](O)CN6C(=O)[C@@H](NC(=O)C6(F)CC6)C(C)(C)C)nc5)ccc43)ccc2nc1N]

Example A150 was synthesized following the same procedures as described in Example A39 (1.4 mg, 7% yield over 2 steps) as a white solid. LC-MS (ESI): mass calcd. for C₅₉H₆₄FN₉O₉S, 1093.45; m/z found, 1094.4 [M+H]⁺. ¹H NMR (400 MHz, DMSO-d₆) δ 8.99 (s, 1H), 8.92 (s, 1H), 8.74-8.67 (m, 2H), 8.27-8.24 (m, 1H), 8.08 (d, J=8.3 Hz, 2H), 7.91 (s, 1H), 7.76-7.60 (m, 3H), 7.56 (d, J=8.1 Hz, 1H), 7.54-7.51 (m, 1H), 7.48 (s, 1H), 7.43-7.34 (m, 3H), 7.33 (s, 1H), 7.31-7.28 (m, 1H), 5.24-5.12 (m, 1H), 4.77-4.66 (m, 2H), 4.59 (d, J=9.3 Hz, 1H), 4.54-4.50 (m, 1H), 4.49-4.47 (m, 2H), 4.45-4.41 (m, 1H), 4.38-4.33 (m, 2H), 3.70-3.67 (m, 2H), 3.64-3.58 (m, 6H), 3.52-3.49 (m, 2H), 3.36-3.35 (m, 1H), 2.66 (s, 3H), 2.42 (s, 3H), 2.28 (s, 3H), 2.11-2.07 (m, 1H), 1.94-1.88 (m, 1H), 1.38-1.33 (m, 2H), 1.23-1.21 (m, 2H), 0.95 (s, 9H).

### Example A151. 2-amino-N-((S)-6-(6-(4-(5-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)pent-4-yn-1-yl)piperazine-1-carbonyl)pyridin-3-yl)-2,3-dihydrobenzofuran-3-yl)-N,3-dimethylquinoline-6-carboxamide

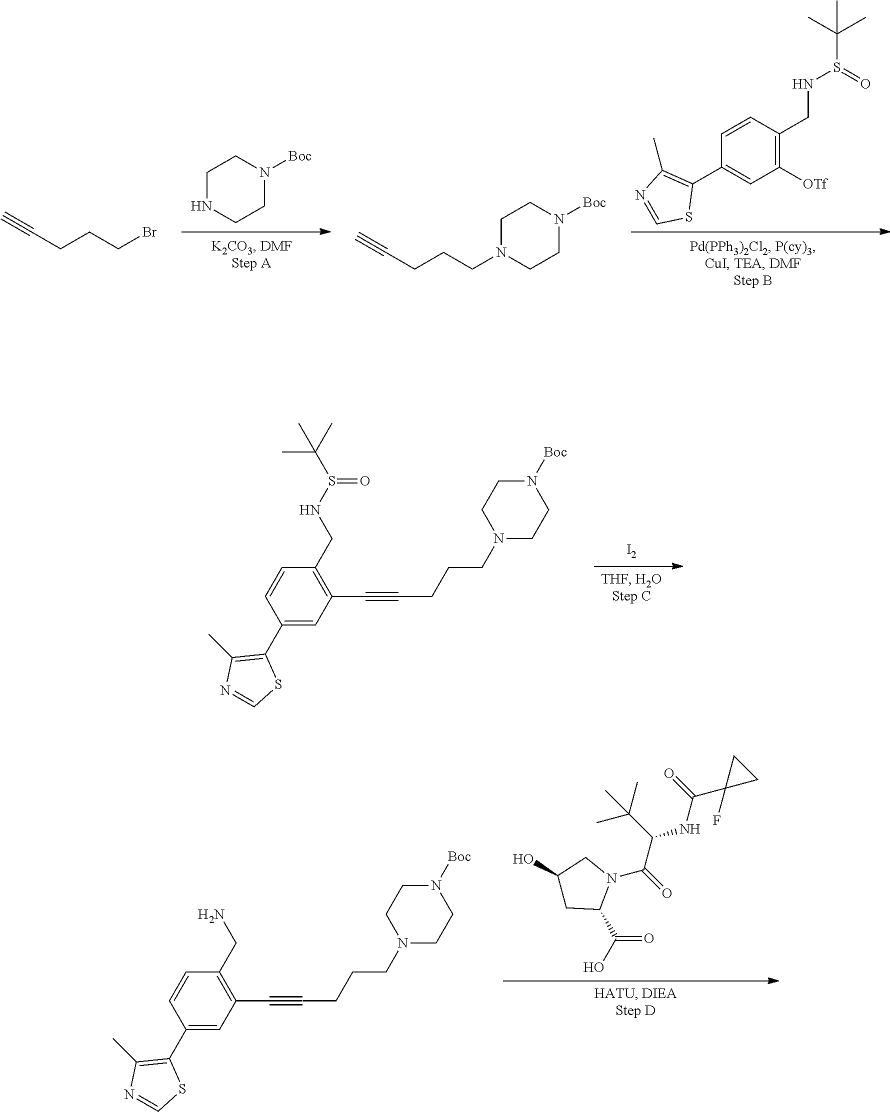

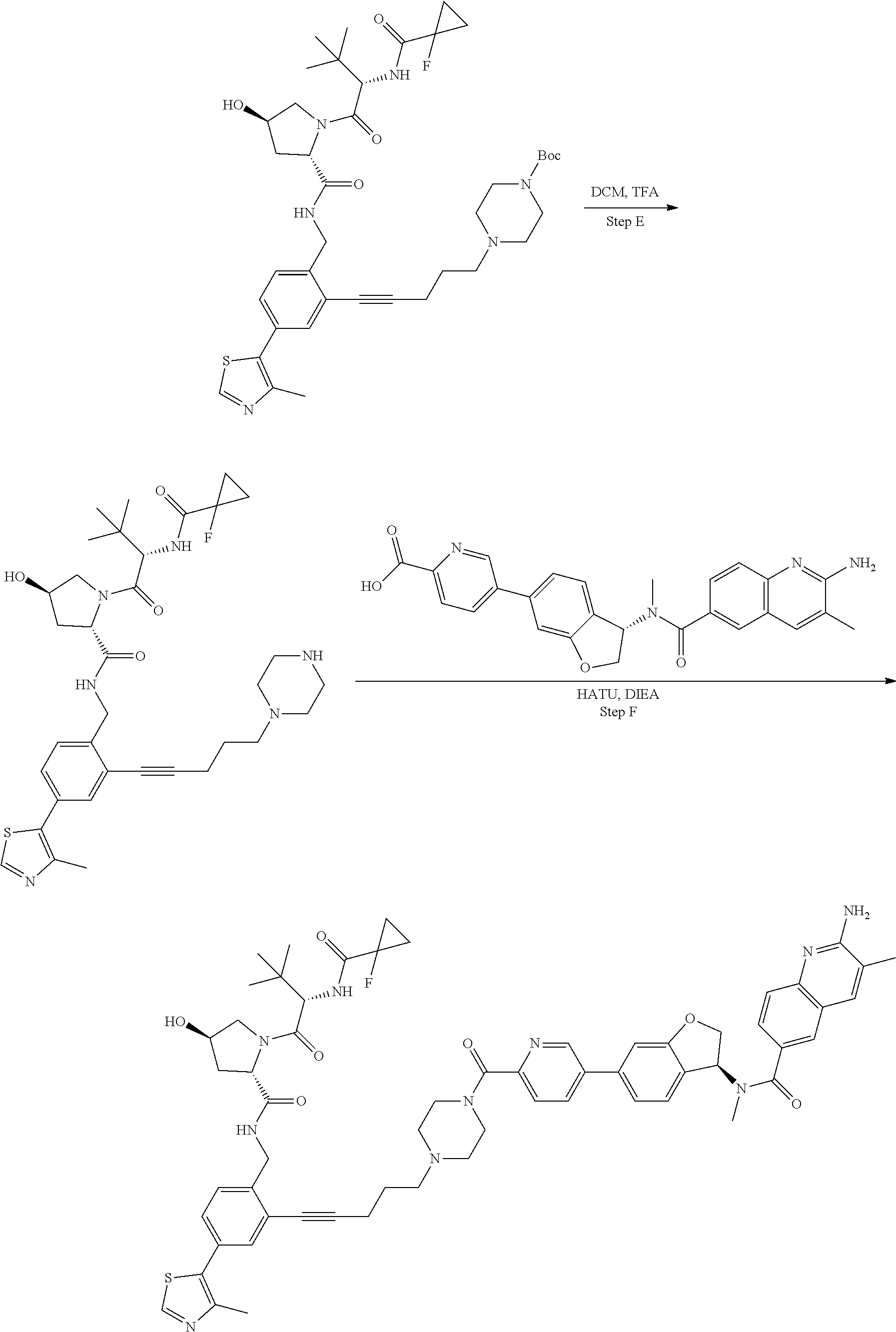

Step A: tert-butyl 4-(pent-4-yn-1-yl)piperazine-1-carboxylate

To a solution of 5-bromopent-1-yne (500 mg, 3.4 mmol, 1 eq.) in DMF (10 mL) was added tert-butyl piperazine-1-carboxylate (633 mg, 3.4 mmol, 1 eq.), K₂CO₃ (1.41 g, 10.2 mmol, 3.0 eq.) and Potassium iodide (56.5 mg, 340 μmol, 0.1 eq.) at room temperature. The mixture was then stirred at 55° C. for 16 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% MeOH in DCM~7% MeOH in DCM) to afford tert-butyl 4-(pent-4-yn-1-yl)piperazine-1-carboxylate (700 mg, 82% yield) as a yellow oil.

LC-MS (ESI): mass calcd. For C₁₄H₂₄N₂O₂, 252.2; m/z found, 253.2 [M+H]⁺.

Step B-F: 2-amino-N-(S)-6-(6-(4-(5-(2-(((25, 4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)pent-4-yn-1-yl)piperazine-1-carbonyl)pyridin-3-yl)-2,3-dihydrobenzofuran-3-yl)-N,3-dimethylquinoline-6-carboxamide

Example A151 was synthesized following the same procedures as described in Example A141 (4.2 mg, 2% yield over 5 steps) as a white solid. LC-MS (ESI): mass calcd. for C₆₁H₆₇FN₁₀O₇S, 1102.5/z found, 1103.2 [M+H]⁺, ¹H NMR (400 MHz, DMSO-d₆) δ 9.01 (s, 1H), 8.93 (s, 1H), 8.91-8.72 (m, 2H), 8.71-8.66 (m, 1H), 8.32 (s, 1H), 8.29-8.25 (m, 1H), 8.04 (s, 1H), 7.87 (d, J=8.7 Hz, 1H), 7.77 (d, J=8.1 Hz, 2H), 7.60-7.52 (m, 2H), 7.47 (d, J=1.7 Hz, 1H), 7.43-7.37 (m, 2H), 7.35 (s, 1H), 7.31-7.25 (m, 1H), 4.81 (s, 1H), 4.74-4.66 (m, 2H), 4.59 (d, J=9.3 Hz, 1H), 4.56-4.48 (m, 2H), 4.45 (d, J=6.0 Hz, 1H), 4.41 (d, J=5.6 Hz, 1H), 4.38-4.33 (m, 2H), 4.31-4.23 (m, 2H), 3.67-3.61 (m, 2H), 3.57-3.45 (m, 2H), 3.34-3.27 (m, 2H), 3.23-3.15 (m, 2H), 2.71-2.61 (m, 5H), 2.44 (s, 3H), 2.33 (s, 3H), 2.14-2.06 (m, 1H), 2.04-1.96 (m, 2H), 1.95-1.87 (m, 1H), 1.44-1.31 (m, 2H), 1.25-1.18 (m, 2H), 0.95 (s, 9H).

### Example A152. N-(cyclopropylmethyl)-N-((S)-6-(6-((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclobutyl)carbamoyl) pyridin-3-yl)-2,3-dihydrobenzofuran-3-yl)-3-methylquinoline-6-carboxamide

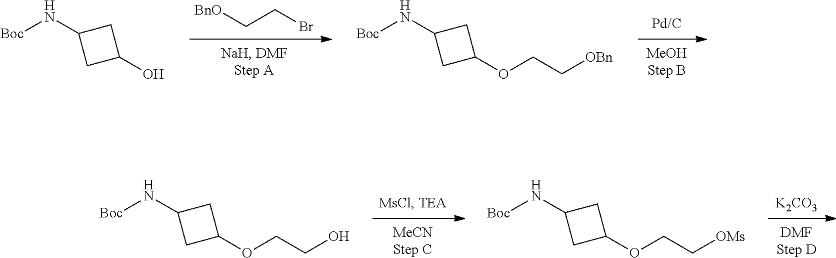

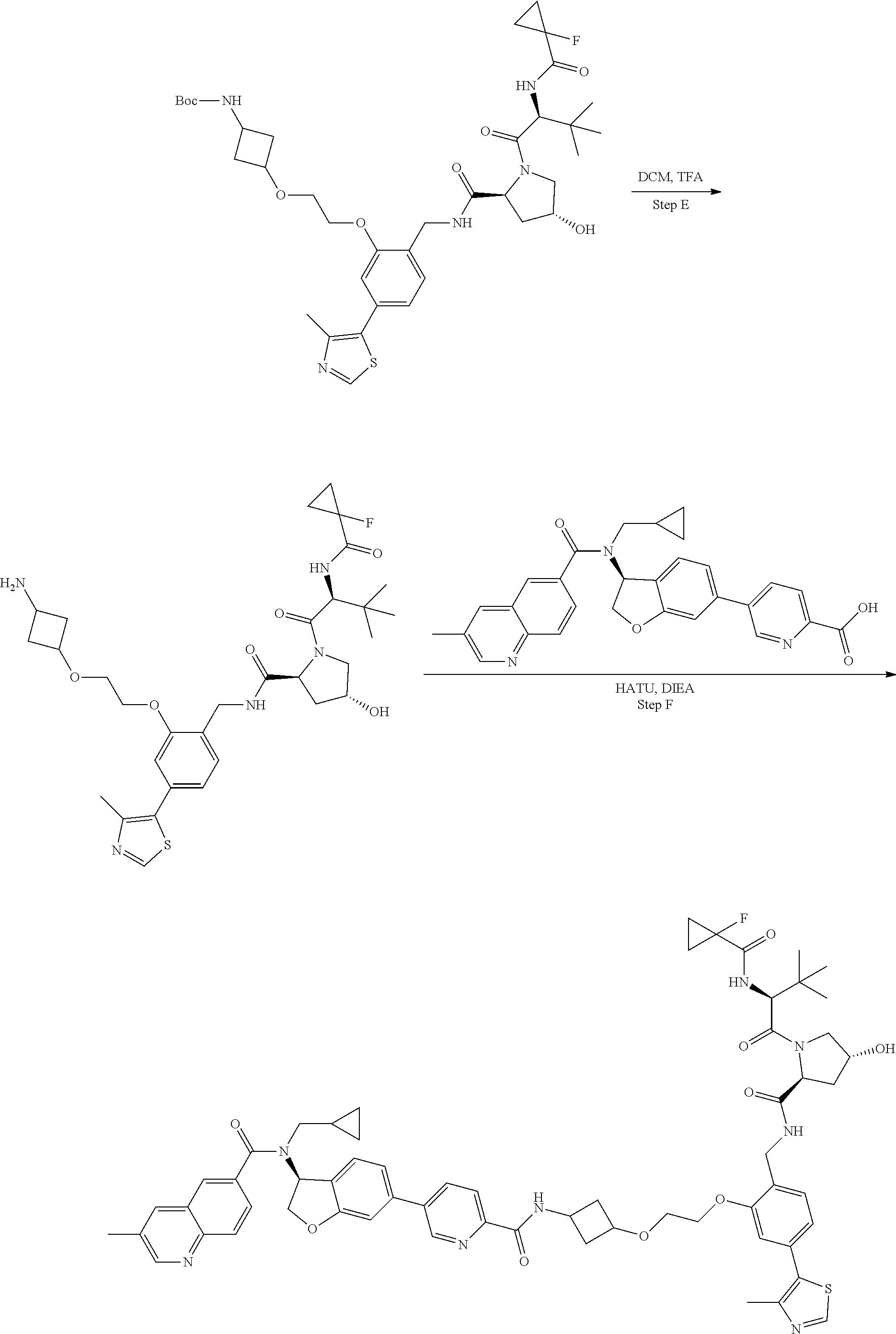

Example A152 was synthesized following the same procedures as described in Example A29 (4.2 mg, 1% yield over 6 steps) as a white solid. LC-MS (ESI): mass calcd. for C₆₁H₆₇FN₈O₉S, 1106.5/z found, 1107.3 [M+H]⁺. ¹H NMR (400 MHz, DMSO-d₆) δ 9.07-8.95 (m, 2H), 8.91 (d, J=9.2 Hz, 2H), 8.56-8.46 (m, 1H), 8.33 (s, 1H), 8.27 (d, J=8.1 Hz, 1H), 8.13-8.03 (m, 3H), 7.81 (d, J=8.0 Hz, 1H), 7.62 (d, J=7.8 Hz, 1H), 7.44-7.36 (m, 2H), 7.35-7.24 (m, 2H), 7.06 (d, J=4.7 Hz, 1H), 6.97 (d, J=7.8 Hz, 1H), 4.91-4.71 (m, 2H), 4.59 (d, J=9.1 Hz, 1H), 4.55-4.50 (m, 1H), 4.37-4.30 (m, 2H), 4.26 (d, J=5.4 Hz, 1H), 4.22-4.17 (m, 2H), 4.11-4.03 (m, 1H), 3.88-3.82 (m, 1H), 3.73-3.70 (m, 2H), 3.68-3.65 (m, 1H), 3.65-3.63 (m, 1H), 3.62-3.61 (m, 1H), 3.59-3.58 (m, 1H), 3.50-3.46 (m, 1H), 3.20-3.14 (m, 1H), 3.12-3.04 (m, 1H), 2.64-2.57 (m, 2H), 2.53 (s, 3H), 2.47 (s, 3H), 2.17-2.08 (m, 2H), 1.99-1.86 (m, 1H), 1.41-1.30 (m, 2H), 1.24-1.20 (m, 2H), 0.95 (s, 9H), 0.91-0.71 (m, 1H), 0.42-0.22 (m, 2H), −0.16−0.27 (m, 1H).

### Example A153. 2-amino-N-(cyclopropylmethyl)-N-((S)-6-(6-((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclobutyl)carbamoyl) pyridin-3-yl)-2,3-dihydrobenzofuran-3-yl)-3-methylquinoline-6-carboxamide

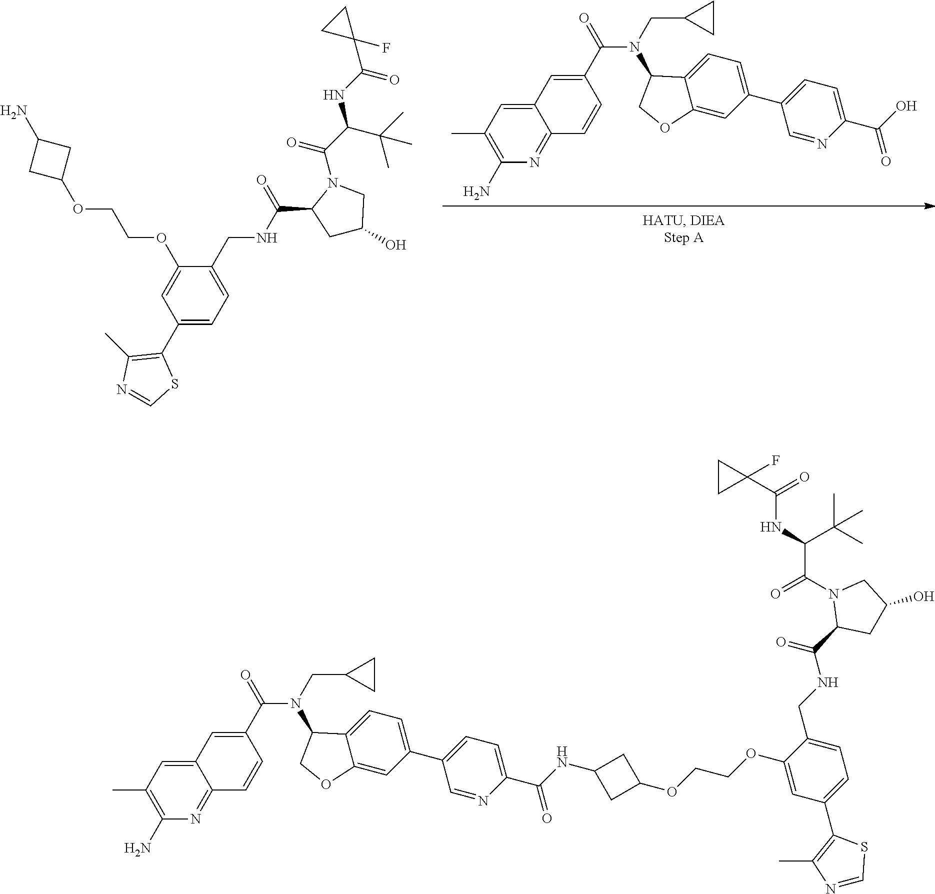

Example A153 was synthesized following the same procedures as described in Example A29 (2 mg, 7% yield) as a white solid. LC-MS (ESI): mass calcd, for C₆₁H₆₈FNO₉S, 1121.5/z found, 1122.3 [M+H]⁺. ¹H NMR (400 MHz, DMSO-d₆) δ 9.29-9.10 (m, 3H), 8.92 (s, 2H), 8.76-8.65 (m, 1H), 8.53 (s, 1H), 8.47 (d, J=8.2 Hz, 1H), 8.31-8.25 (m, 1H), 8.21 (s, 1H), 8.04 (d, J=8.7 Hz, 1H), 7.97 (d, J=8.4 Hz, 1H), 7.80 (d, J=7.8 Hz, 1H), 7.66-7.57 (m, 2H), 7.56-7.42 (m, 2H), 7.29-7.25 (m, 1H), 7.18 (d, J=7.7 Hz, 1H), 5.06-4.91 (m, 2H), 4.80 (d, J=9.2 Hz, 1H), 4.75-4.71 (m, 1H), 4.57-4.55 (m, 1H), 4.53 (d, J=5.9 Hz, 1H), 4.46 (d, J=5.3 Hz, 1H), 4.43-4.41 (m, 1H), 4.40-4.39 (m, 1H), 4.31-4.29 (m, 2H), 4.27-4.26 (m, 2H), 4.08-4.06 (m, 1H), 3.94-3.90 (m, 2H), 3.87-3.79 (m, 2H), 3.39-3.32 (m, 1H), 3.31-3.23 (m, 1H), 2.87-2.76 (m, 2H), 2.68 (d, J=2.1 Hz, 3H), 2.53 (s, 3H), 2.37-2.28 (m, 2H), 2.23-2.08 (m, 1H), 1.63-1.51 (m, 2H), 1.46-1.39 (m, 2H), 1.16 (s, 9H), 1.12-0.95 (m, 1H), 0.63-0.44 (m, 2H), 0.05-0.07 (m, 1H).

### Example A154. 2-amino-N-((S)-6-(6-(4-(4-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)piperazin-1-yl)piperidine-1-carbonyl)pyridin-3-yl)-2,3-dihydrobenzofuran-3-yl)-N,3-dimethylquinoline-6-carboxamide

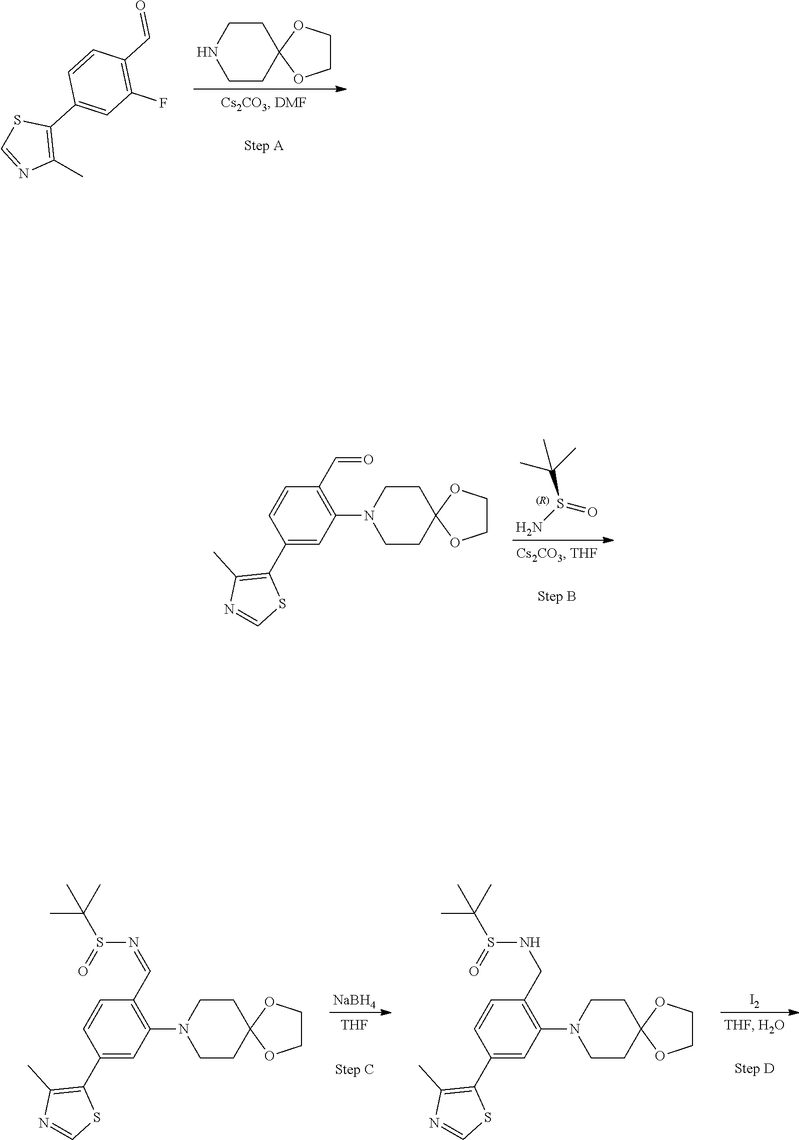

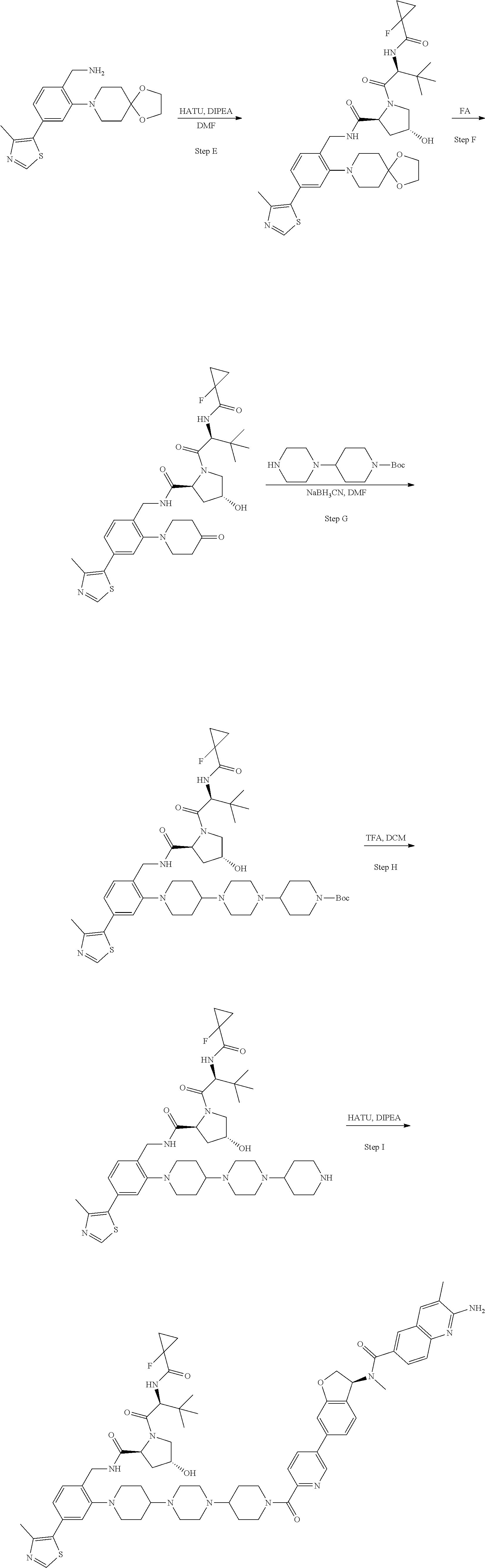

Step A: 4-(4-methylthiazol-5-yl)-2-(1,4-dioxa-8-azaspiro[4.5]decan-8-yl)benzaldehyde

To a solution of 2-fluoro-4-(4-methylthiazol-5-yl)benzaldehyde (2.0 g, 9.04 mmol, 1.0 eq.) in DMSO (30 mL) was added 1,4-dioxa-8-azaspiro[4.5]decane (1.9 g, 13.6 mmol, 1.5 eq.) and K₂CO₃ (3.7 g, 27.1 mmol, 3.0 eq.) at room temperature. The mixture was stirred at 80° C. for 16 hours. The resulting solution was diluted with H₂O (40 mL), then extracted with EtOAc (20 mL×3) and the layers were separated. The organic layer was washed with brine (20 mL), dried over Na₂SO₄ and concentrated under reduced pressure. The residue was purified by flash chromatography (100-200 mesh silica gel, 0-20% EtOAc in PE) to afford 4-(4-methylthiazol-5-yl)-2-(1,4-dioxa-8-azaspiro[4.5]decan-8-yl)benzaldehyde (1.0 g, 32% yield) as a yellow solid.

LC-MS (ESI): mass calcd. For C₁₅H₂₀N₂O₃S, 344.12; m/z found, 345.3 [M+H]⁺.

Step B: (Z)-2-methyl-N-(4-(4-methylthiazol-5-yl)-2-(1,4-dioxa-8-azaspiro[4.5]decan-8-yl)benzylidene)propane-2-sulfinamide

To a mixture of 4-(4-methylthiazol-5-yl)-2-(1,4-dioxa-8-azaspiro[4.5]decan-8-yl)benzaldehyde (1.0 g, 2.90 mmol, 1.0 eq.) and 2-methylpropane-2-sulfinamide (530 mg, 4.36 mmol, 1.5 eq.) in THF (15 mL) was added Cs₂CO₃ (1.9 g, 5.81 mmol, 2.0 eq.) at room temperature. Then was stirred at 60° C. for 6 hours. The mixture was filtered and purified by flash chromatography (100-200 mesh silica gel, 0-80% EtOAc in PE) to afford the (Z)-2-methyl-N-(4-(4-methylthiazol-5-yl)-2-(1,4-dioxa-8-azaspiro[4.5]decan-8-yl)benzylidene)propane-2-sulfinamide (1.0 g, 77% yield) as yellow oil. LC-MS (ESI): mass calcd. For C₂₂H₂₉N₃O₃S₂, 447.17; m/z found, 448.4 [M+H]⁺.

Step C: 2-methyl-N-(4-(4-methylthiazol-5-yl)-2-(1,4-dioxa-8-azaspiro[4.5]decan-8-yl)benzyl)propane-2-sulfinamide

Sodium Tetrahydroborate (85 mg, 2.23 mmol, 1.0 eq.) was slowly added to a solution of (Z)-2-methyl-N-(4-(4-methylthiazol-5-yl)-2-(1,4-dioxa-8-azaspiro[4.5]decan-8-yl)benzylidene)propane-2-sulfinamide (1.0 g, 2.23 mmol, 1.0 eq.) in MeOH (20 mL) and continuously stirred at room temperature for 2 hours, then cold saturated aqueous NH₄Cl solution was added and the mixture extracted with DCM (30 mL×3). The organic layer was washed with brine, dried over Na₂SO₄ and concentrated in vacuo. The residue was purified by chromatography (100-200 mesh silica gel, 0-80% EtOAc in PE) to afford to afford 2-methyl-N-(4-(4-methylthiazol-5-yl)-2-(1,4-dioxa-8-azaspiro[4.5]decan-8-yl)benzyl)propane-2-sulfinamide (800 mg, 80% yield) as a yellow oil. LC-MS (ESI): mass calcd. For C₂₂H₃₁N₃O₃S₂, 449.18; m/z found, 450.4 [M+H]⁺.

Step D: (4-(4-methylthiazol-5-yl)-2-(1,4-dioxa-8-azaspiro[4.5]decan-8-yl)phenyl)methanamine

To a flask containing 2-methyl-N-(4-(4-methylthiazol-5-yl)-2-(1,4-dioxa-8-azaspiro[4.5]decan-8-yl)benzyl)propane-2-sulfinamide (500 mg, 1.1 mmol, 1.0 eq.) in DCM (6 mL) was added HCl (6 mL, 4 N in 1,4-dioxane) at room temperature. The reaction was stirred at room temperature for 2 hours. The residue was concentrated under reduced pressure to give (4-(4-methylthiazol-5-yl)-2-(1,4-dioxa-8-azaspiro[4.5]decan-8-yl)phenyl)methanamine (300 mg, 78% yield) as a solid. LC-MS (ESI): mass calcd. For C₁₈H₂₃N₃O₂S, 345.15; m/z found, 346.1 [M+H]⁺.

Step E: (2S,4R)-1-((S)-2-(1-fluorocyclopropane-1-carboxamido)-3,3-dimethylbutanoyl)-4-hydroxy-N-(4-(4-methylthiazol-5-yl)-2-(1,4-dioxa-8-azaspiro[4.5]decan-8-yl)benzyl)pyrrolidine-2-carboxamide

To a stirred mixture of (4-(4-methylthiazol-5-yl)-2-(1,4-dioxa-8-azaspiro[4.5]decan-8-yl)phenyl)methanamine (300 mg, 0.9 mmol, 1.0 eq.) in DMF (10 mL) were added (2S,4R)-1-((S)-2-(1-fluorocyclopropane-1-carboxamido)-3,3-dimethylbutanoyl)-4-hydroxypyrrolidine-2-carboxylic acid (300 mg, 0.9 mmol, 1 eq.) and HATU (660 mg, 1.0 mmol, 1.2 eq.). DIEA (560 mg, 0.8 mL, 4.3 mmol, 5.0 eq.) was then added and the mixture was stirred at room temperature for 2 hours. The reaction mixture was diluted with water (50 mL), extracted with EtOAc (50 mL×3), then dried over anhydrous Na₂SO₄, filtered and evaporated. The crude product was purified by flash silica chromatography (100-200 mesh silica gel, 0-2% MeOH in DCM) to afford (2S,4R)-1-((S)-2-(1-fluorocyclopropane-1-carboxamido)-3,3-dimethylbutanoyl)-4-hydroxy-N-(4-(4-methylthiazol-5-yl)-2-(1,4-dioxa-8-azaspiro[4.5]decan-8-yl)benzyl)pyrrolidine-2-carboxamide (360 mg, 63% yield) as a yellow solid. LC-MS (ESI): mass calcd. For C₃₃H₄₄FN₅O₆S, 657.30; m/z found, 658.6 [M+H]⁺.

Step F: (2S, 4R)-1-((S)-2-(1-fluorocyclopropane-1-carboxamido)-3,3-dimethylbutanoyl)-4-hydroxy-N-(4-(4-methylthiazol-5-yl)-2-(4-oxopiperidin-1-yl)benzyl)pyrrolidine-2-carboxamide

To a flask containing (2S,4R)-1-((S)-2-(1-fluorocyclopropane-1-carboxamido)-3,3-dimethylbutanoyl)-4-hydroxy-N-(4-(4-methylthiazol-5-yl)-2-(1,4-dioxa-8-azaspiro[4.5]decan-8-yl)benzyl)pyrrolidine-2-carboxamide (500 mg, 760 μmol, 1.0 eq.) in THF (8 mL) was added HCl (8 mL, 1 N) at room temperature. The reaction was stirred at 50° C. for 6 hours. The residue was concentrated under reduced pressure to give 1-fluoro-N-((S)-1-((2S,4R)-4-hydroxy-2-(((4-(4-methylthiazol-5-yl)-2-(4-oxopiperidin-1-yl)benzyl) amino)methyl) pyrrolidin-1-yl)-3,3-dimethyl-1-oxobutan-2-yl)cyclopropane-1-carboxamide (400 mg, 88% yield) as a yellow solid. LC-MS (ESI): mass calcd. For C31H40FN₅O₅S, 613.27; m/z found, 614.2 [M+H]⁺.

Step G-I: 2-amino-N-(S)-6-(6-(4-(4-(1-(2-(((28,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)piperazin-1-yl)piperidine-1-carbonyl)pyridin-3-yl)-2,3-dihydrobenzofuran-3-yl)-N,3-dimethylquinoline-6-carboxamide

Example A154 was synthesized following the same procedures as described in Example A107 (4.7 mg, 15% yield over 3 steps) as a white solid. LC-MS (ESI): mass calcd. for C₆₆H₇₉FN₁₂O₇S, 1202.59; m/z found, 1107.4 [M+H]⁺. ¹H NMR (400 MHz, DMSO-d₆) δ 8.99 (s, 1H), 8.92-8.88 (m, 1H), 8.56-8.47 (m, 1H), 8.25-8.20 (m, 1H), 8.13 (s, 1H), 7.90-7.80 (m, 2H), 7.65 (d, J=8.1 Hz, 1H), 7.61-7.52 (m, 3H), 7.44 (d, J=8.2 Hz, 1H), 7.38 (d, J=7.8 Hz, 1H), 7.33 (s, 1H), 7.29-7.24 (m, 1H), 7.16-7.11 (m, 2H), 6.90-6.47 (m, 2H), 5.20-5.16 (m, 1H), 4.81-4.29 (m, 10H), 3.95-3.76 (m, 2H), 3.68-3.60 (m, 3H), 3.51-3.46 (m, 2H), 3.20-2.98 (m, 8H), 2.89-2.71 (m, 5H), 2.68 (s, 3H), 2.46 (s, 3H), 2.24 (s, 3H), 2.12-2.06 (m, 2H), 1.95-1.87 (m, 2H), 1.84-1.73 (m, 2H), 1.51-1.33 (m, 4H), 1.24-1.20 (m, 2H), 0.97 (s, 9H).

### Example A155. N1-(6-amino-5-(difluoromethyl)pyridin-3-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) ethoxy)ethyl)carbamoyl)pyridin-3-yl)-2,3-dihydrobenzofuran-3-yl)-N2-methyloxalamide

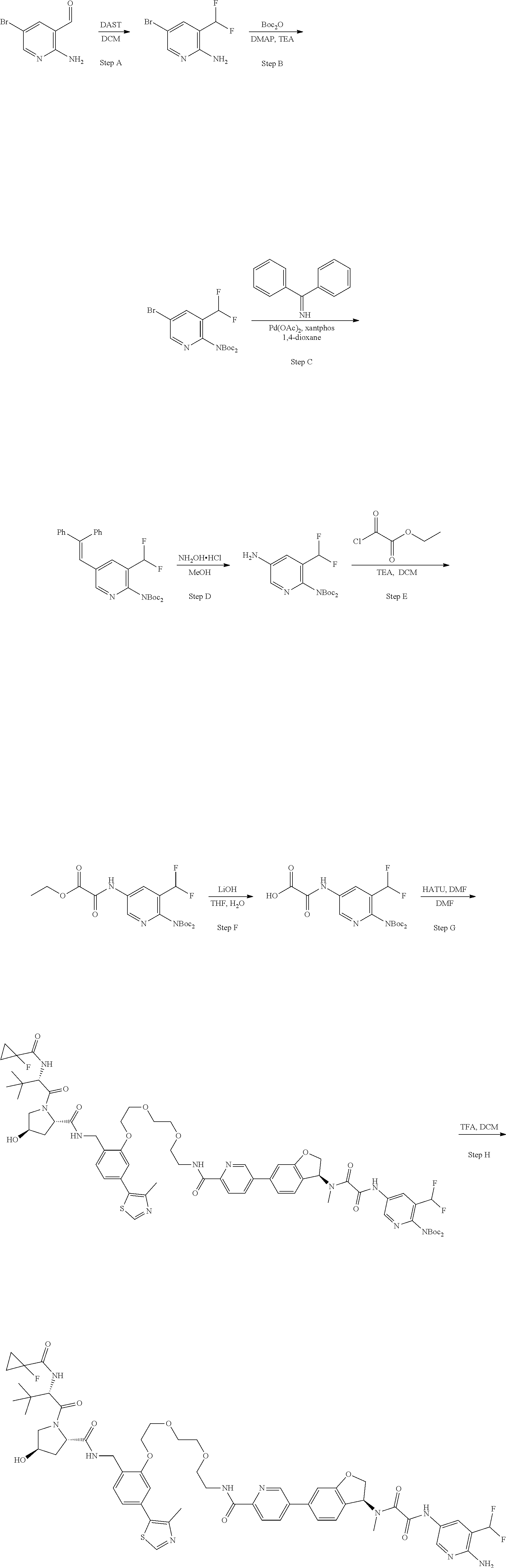

Step A: 5-bromo-3-(difluoromethyl)pyridin-2-amine

To a solution of 2-amino-5-bromonicotinaldehyde (400 mg, 2.0 mmol, 1.0 eq.) in DCM (20 mL) was added DAST (1.6 g, 1.3 mL, 10.0 mmol, 5.0 eq.) dropwise at 0° C. The resulting mixture was stirred at room temperature for 15 hours. The reaction mixture was quenched with aq. NaHCO₃. extracted with DCM (30 mL×2), dried over anhydrous Na₂SO₄ and concentrated. The residue was purified by column chromatography (silica gel, 0% EtOAc in PE~20% EtO Ac in PE) to afford 5-bromo-3-(difluoromethyl)pyridin-2-amine (120 mg, 22% yield) as a yellow solid. LC-MS (ESI): mass calcd. For C₆H₅BrF₂N2, 223.02; m/z found, 224/226 [M+H]⁺.

Step B: di-tert-butyl (5-bromo-3-(difluoromethyl)pyridin-2-yl)carbamate

To a solution of 5-bromo-3-(difluoromethyl)pyridin-2-amine (220 mg, 1.0 mmol, 1.0 eq.) and di-tert-butyl dicarbonate (650 mg, 3 mmol, 3 eq.) in DCM (10 mL) were added DMAP (20 mg, cat.) and TEA (300 mg, 0.4 mL, 3.0 mmol, 3.0 eq.). The resulting mixture was stirred at room temperature for 1 hour. The reaction mixture was poured into water (30 mL) and then extracted with DCM (30 mL×2). The combined organic layers were washed with brine, dried over anhydrous Na₂SO₄ and concentrated. The residue was purified by column chromatography (silica gel, 0% EtOAc in PE~10% EtOAc in PE) to afford di-tert-butyl(2S,4R)-2-((2-(4-(dimethoxymethyl)piperidin-1-yl)-4-fluorobenzyl)carbamoyl)-4-hydroxypyrrolidine-1-carboxylate (195 mg, 50% yield) as a white solid. LC-MS (ESI): mass calcd. For C₁₆H₂₁F₂N₂O₄, 423.25; m/z found, 424/426 [M+H]⁺.

Step C: di-tert-butyl (3-(difluoromethyl)-5-((diphenylmethylene)amino)pyridin-2-yl)carbamate

To a stirred mixture of di-tert-butyl(2S,4R)-2-((2-(4-(dimethoxymethyl)piperidin-1-yl)-4-fluorobenzyl)carbamoyl)-4-hydroxypyrrolidine-1-carboxylate (210 mg, 500 μmol, 1.0 eq.) and diphenylmethanimine (180 mg, 1.0 mmol, 2.0 eq.) in 1,4-dioxane (1 mL) were added Cs₂CO₃ (326 mg, 1.0 mmol, 2 eq.), Pd (OAc) 2 (12 mg, 50 μmol, 0.1 eq.) and xantphos (25 mg, 50 μmol, 0.1 eq.). The resulting mixture was stirred at 100° C. for 15 hours. The reaction mixture was poured into water (20 mL) and then extracted with EtOAc (20 mL×2). The combined organic layers were washed with brine, dried over anhydrous Na₂SO₄ and concentrated. The residue was purified by column chromatography (silica gel, 0% EtOAc in PE~50% EtOAc in PE) to afford di-tert-butyl (3-(difluoromethyl)-5-((diphenylmethylene)amino)pyridin-2-yl)carbamate (190 mg, 73% yield) as a yellow solid. LC-MS (ESI): mass calcd. For C29H31F₂N304, 523.58; m/z found, 524 [M+H]⁺.

Step D: di-tert-butyl (5-amino-3-(difluoromethyl)pyridin-2-yl)carbamate

To a solution of di-tert-butyl (3-(difluoromethyl)-5-((diphenylmethylene)amino)pyridin-2-yl)carbamate (190 mg, 360 μmol, 1.0 eq.) in MeOH (2 mL) was added hydroxylamine hydrochloride (75 mg, 1.1 mmol, 3 eq.). The resulting mixture was stirred at room temperature for 1 hour. The reaction mixture was poured into water (20 mL) and then extracted with EtOAc (20 mL×2). The combined organic layers were washed with brine, dried over anhydrous Na₂SO₄ and concentrated. The residue was purified by column chromatography (silica gel, 0% EtOAc in PE~50% EtOAc in PE) to afford di-tert-butyl (5-amino-3-(difluoromethyl)pyridin-2-yl)carbamate (60 mg, 63% yield) as a white solid. LC-MS (ESI): mass calcd. For C₁₁H₁₅F₂N302, 259.26; m/z found, 260 [M+H]⁺.

Steps E-H: N1-(6-amino-5-(difluoromethyl)pyridin-3-yl)-N2-(S)-6-(6-(2-(2-(2-(2-(((28, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N2-methyloxalamide

Example A155 was synthesized following the same procedures as described in Example A44 (3.1 mg, 18% yield over 4 steps) as a white solid. LC-MS (ESI): mass calcd. for C₅₅H₆₃F₃N₁₀O₁₁S, 1129.34; m/z found, 1130 [M+H]⁺. ¹H NMR (400 MHz, DMSO-d₆) δ 10.84 (d, J=55.1 Hz, 1H), 8.96 (s, 1H), 8.91 (s, 1H), 8.77-8.67 (m, 1H), 8.52-8.43 (m, 1H), 8.40-8.28 (m, 1H), 8.26 (d, J=8.0 Hz, 1H), 8.14-7.99 (m, 2H), 7.61-7.38 (m, 3H), 7.37-7.17 (m, 3H), 7.11-6.88 (m, 3H), 6.76-6.30 (m, 1H), 6.29-5.89 (m, 1H), 4.81-4.63 (m, 2H), 4.59 (d, J=9.2 Hz, 1H), 4.51 (t, J=8.2 Hz, 1H), 4.35-4.27 (m, 2H), 4.25-4.15 (m, 3H), 3.81-3.79 (m, 2H), 3.68-3.62 (m, 8H), 2.93-2.80 (m, 2H), 2.74-2.62 (m, 3H), 2.43 (s, 3H), 2.10-2.05 (m, 1H), 1.94-1.89 (m, 1H), 1.39-1.32 (m, 2H), 1.26-1.21 (m, 2H), 0.95 (s, 9H).

### Example A156. 2-amino-N-((R)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl)pyridin-3-yl)-2,3-dihydrobenzofuran-3-yl)-N,3-dimethylquinoline-6-carboxamide

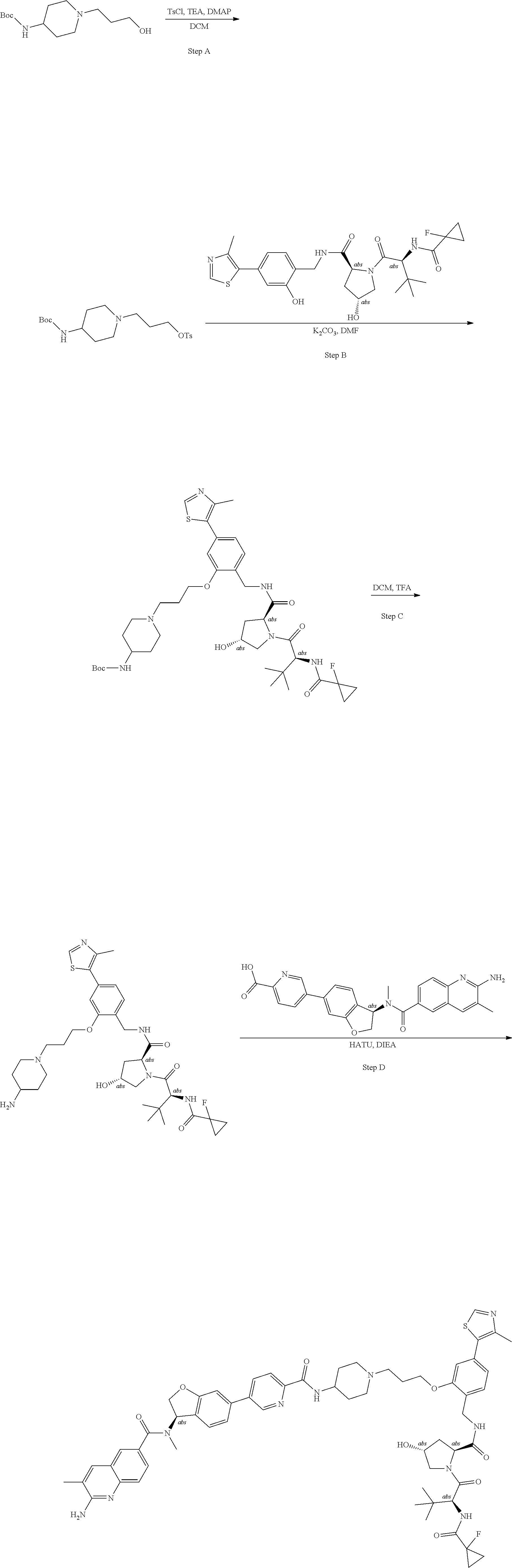

Example A156 was synthesized following the same procedures as described in Example A29 (3.6 mg, 2% yield over 4 steps) as a white solid. LC-MS (ESI): mass calcd. for C₆₀H₆₉FN₁₀O₇S, 1108.5/z found, 1109.3 [M+H]⁺. ¹H NMR (400 MHz, DMSO-d₆) δ 8.99 (s, 1H), 8.93 (s, 1H), 8.55 (d, 1H), 8.52-8.46 (m, 1H), 8.32-8.23 (m, 1H), 8.18 (s, 1H), 8.08 (d, J=8.2 Hz, 1H), 7.80 (d, J=12.8 Hz, 2H), 7.59-7.47 (m, 3H), 7.43-7.37 (m, 2H), 7.34 (s, 1H), 7.29 (d, J=6.8 Hz, 1H), 7.03-6.99 (m, 1H), 6.96 (d, J=7.8 Hz, 1H), 6.50 (s, 2H), 5.17 (s, 1H), 4.71-4.66 (m, 1H), 4.60 (d, J=9.3 Hz, 1H), 4.55-4.49 (m, 1H), 4.36 (s, 1H), 4.31-4.20 (m, 2H), 4.12-4.06 (m, 2H), 3.88-3.76 (m, 1H), 3.68-3.59 (m, 2H), 3.46-3.36 (m, 3H), 2.96-2.83 (m, 2H), 2.67 (s, 3H), 2.47 (s, 3H), 2.23 (s, 3H), 2.12-2.03 (m, 3H), 1.96-1.89 (m, 3H), 1.80-1.75 (m, 2H), 1.72-1.64 (m, 2H), 1.40-1.33 (m, 2H), 1.24-1.20 (m, 2H), 0.96 (s, 9H).

### Example A157. N1-(6-amino-5-cyanopyridin-3-yl)-N2-((S)-6-(6-(4-((1-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N2-methyloxalamide

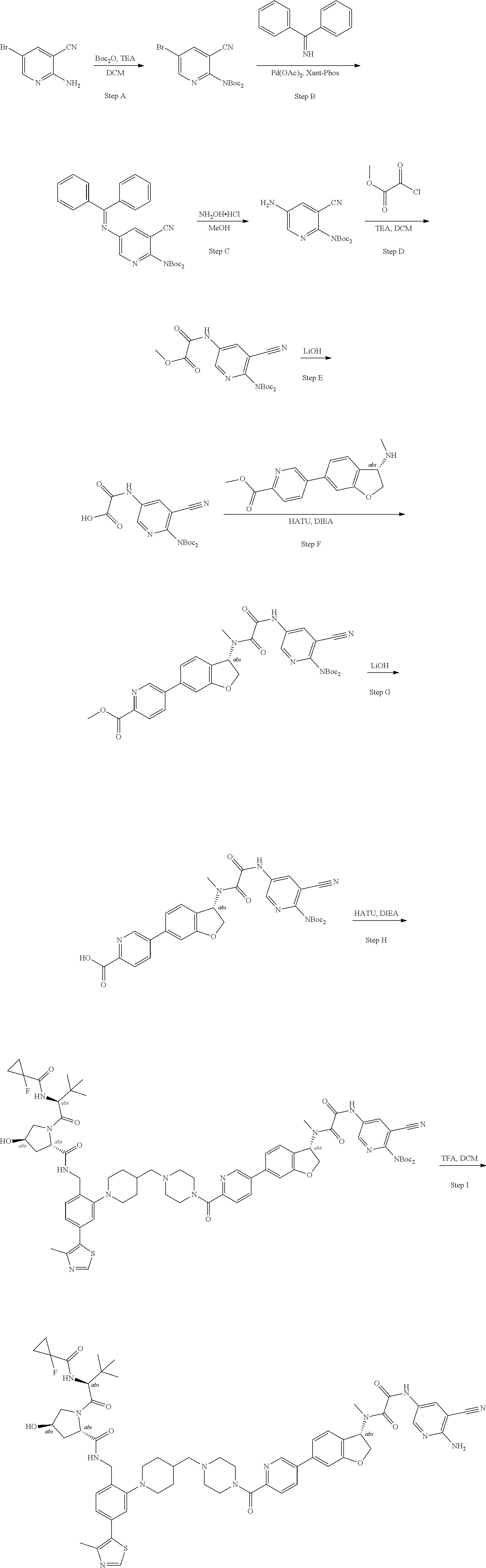

Example A157 was synthesized following the same procedures as described in Example A155 (2.3 mg, 2% yield over 9 steps) as a white solid. LC-MS (ESI): mass calcd. For C₅₉H₆₅FN₁₃O₈S, 1137.50; m/z found, 1138.6 [M+H]⁺. ¹H NMR (400 MHz, DMSO-d₆) δ 10.88 (d, J=48.8 Hz, 1H), 9.02-8.92 (m, 2H), 8.51 (s, 1H), 8.42 (dd, J=14.5, 2.6 Hz, 1H), 8.29 (d, J=8.1 Hz, 1H), 8.13 (dd, J=25.4, 2.6 Hz, 1H), 7.78 (d, J=8.1 Hz, 1H), 7.47-7.37 (m, 3H), 7.26 (d, J=9.4 Hz, 1H), 7.16-7.10 (m, 2H), 6.97-6.81 (m, 2H), 4.82-4.70 (m, 1H), 4.68-4.55 (m, 3H), 4.52-4.47 (m, 1H), 4.38-4.34 (m, 2H), 4.32-4.23 (m, 1H), 3.73-3.61 (m, 6H), 3.22-3.09 (m, 8H), 2.77-2.76 (m, 1H), 2.76-2.68 (m, 2H), 2.64-2.60 (m, 2H), 2.46 (s, 3H), 2.12-2.08 (m, 1H), 2.00-1.98 (m, 1H), 1.92-1.86 (m, 3H), 1.48-1.43 (m, 2H), 1.41-1.36 (m, 2H), 1.34-1.28 (m, 2H), 0.98 (s, 9H).

### Example A158. 2-amino-N-((S)-6-(6-(4-((4-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)piperazin-1-yl)methyl)piperidine-1-carbonyl)pyridin-3-yl)-2,3-dihydrobenzofuran-3-yl)-N,3-dimethylquinoline-6-carboxamide

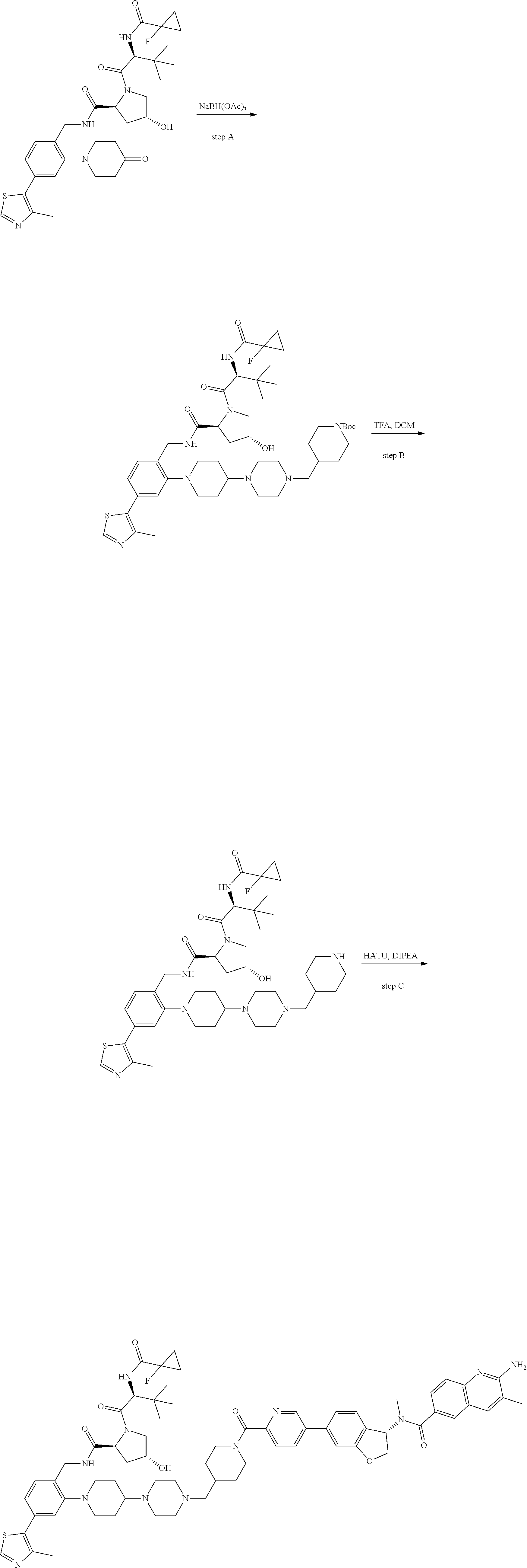

Example A158 was synthesized following the same procedures as described in Example A107 (4.0 mg, 50.4% yield over 3 steps) as a white solid. LC-MS (ESI): mass calcd. for C₆₇H81FN₁₂O₇S, 1216.6/z found, 1217.8 [M+H]⁺. ¹HNMR (400 MHz, DMSO-d₆) δ 9.00-8.97 (m, 1H), 8.90 (d, 1H), 8.50 (t, 1H), 8.20 (dd, 1H), 7.81 (d, J=13.0 Hz, 2H), 7.61 (d, J=8.2 Hz, 1H), 7.58-7.50 (m, 3H), 7.43 (d, J=7.7 Hz, 1H), 7.38 (d, J=7.4 Hz, 1H), 7.33 (s, 1H), 7.28 (dd, J=7.3 Hz, 1H), 7.13-7.09 (m, 2H), 6.50 (s, 2H), 5.17 (d, J=3.6 Hz, 1H), 4.81-4.66 (m, 2H), 4.61 (d, J=9.1 Hz, 1H), 4.49 (t, J=8.1 Hz, 2H), 4.43-4.35 (m, 2H), 4.32-4.25 (m, 1H), 3.76-3.65 (m, 2H), 3.61 (d, J=10.8 Hz, 1H), 3.48-3.43 (m, 1H), 3.28-3.25 (m, 6H), 3.14-3.07 (m, 3H), 3.06-3.03 (m, 1H), 2.84-2.72 (m, 2H), 2.68 (s, 3H), 2.65-2.61 (m, 1H), 2.53 (s, 4H), 2.46 (s, 3H), 2.40-2.36 (m, 2H), 2.23 (s, 3H), 2.17-2.14 (m, 1H), 2.10-2.07 (m, 1H), 1.87-1.82 (m, 3H), 1.65-1.59 (m, 2H), 1.41-1.33 (m, 2H), 1.24-1.21 (m, 2H), 1.14-1.06 (m, 2H), 0.98 (s, 9H).

### Example A159. 2-amino-N-((S)-6-(6-(2-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)-2,7-diazaspiro[3.5]nonane-7-carbonyl)pyridin-3-yl)-2,3-dihydrobenzofuran-3-yl)-N,3-dimethylquinoline-6-carboxamide

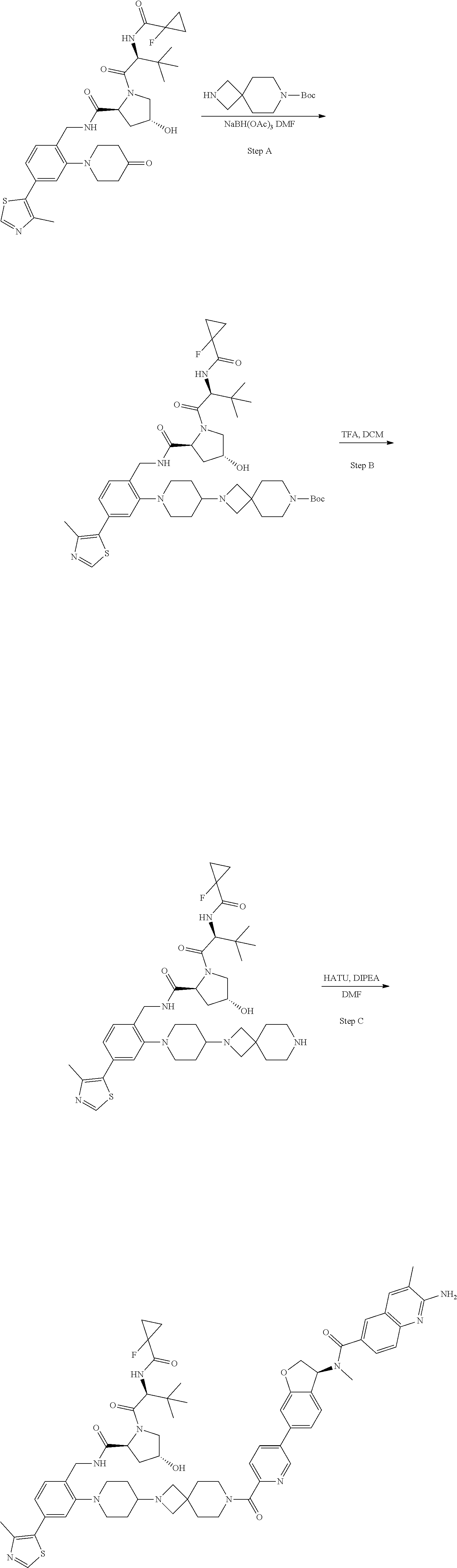

Example A159 was synthesized following the same procedures as described in Example 107 (3.0 mg, 7% yield over 3 steps) as a white solid. LC-MS (ESI): mass calcd. for C₆₄H₇₄FN₁₁O₇S, 1159.5/z found, 1161.1 [M+H]⁺. ¹H NMR (400 MHz, DMSO-d₆) δ 8.97 (s, 1H), 8.91-8.89 (m, 1H), 8.51-8.46 (m, 1H), 8.24-8.16 (m, 2H), 7.82 (s, 1H), 7.79-7.78 (m, 1H), 7.62 (d, J=8.2 Hz, 1H), 7.56-7.49 (m, 3H), 7.42 (d, J=7.9 Hz, 1H), 7.38 (d, J=8.1 Hz, 1H), 7.33 (s, 1H), 7.29-7.26 (m, 1H), 7.11-7.08 (m, 2H), 6.50 (s, 2H), 5.26-5.07 (m, 1H), 4.85-4.63 (m, 3H), 4.60 (d, J=9.3 Hz, 1H), 4.50-4.45 (m, 1H), 4.42-4.24 (m, 4H), 3.68-3.65 (m, 1H), 3.62-3.60 (m, 2H), 3.08-2.97 (m, 8H), 2.70-2.66 (m, 5H), 2.45 (s, 3H), 2.23 (s, 3H), 2.11-2.05 (m, 1H), 1.95-1.89 (m, 1H), 1.79-1.72 (m, 4H), 1.70-1.66 (m, 2H), 1.40-1.32 (m, 4H), 1.23-1.20 (m, 2H), 0.98 (s, 9H).

### Example A160. 2-amino-N-((S)-6-(6-(4-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)piperazine-1-carbonyl)pyridin-3-yl)-2,3-dihydrobenzofuran-3-yl)-N,3-dimethylquinoline-6-carboxamide

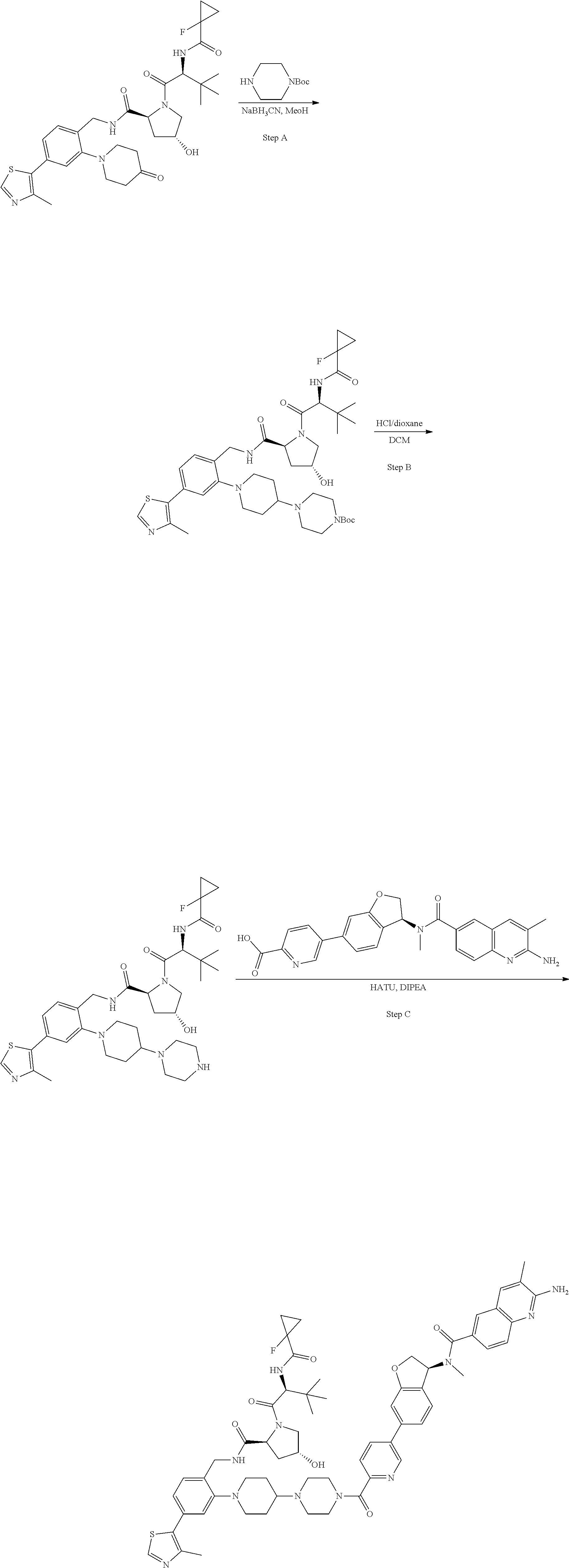

Example A160 was synthesized following the same procedures as described in Example A107 (2.6 mg, 8% yield over 3 steps) as a white solid. LC-MS (ESI): mass calcd. For C₆₁H₇₀FN₁₁O₇S, 1119.52; m/z found, 1120.7 [M+H]⁺. ¹H NMR (400 MHz, DMSO-d₆) δ 9.81 (s, 1H), 9.00 (s, 1H), 8.94 (s, 1H), 8.68 (s, 2H), 8.56-8.50 (m, 1H), 8.33-8.25 (m, 2H), 8.05 (s, 1H), 7.87 (d, J=9.8 Hz, 1H), 7.83-7.76 (m, 2H), 7.57 (d, J=7.5 Hz, 1H), 7.45 (d, J=7.8 Hz, 1H), 7.42-7.34 (m, 2H), 7.29-7.24 (m, 1H), 7.17-7.13 (m, 2H), 4.84-4.65 (m, 3H), 4.62-4.58 (m, 1H), 4.52-4.47 (m, 1H), 4.40-4.30 (m, 4H), 3.69-3.65 (m, 5H), 3.25-3.20 (m, 4H), 2.77-2.65 (m, 6H), 2.54 (s, 3H), 2.46 (s, 3H), 2.33 (s, 3H), 2.18-2.09 (m, 3H), 1.92-1.81 (m, 3H), 1.40-1.33 (m, 2H), 1.24-1.20 (m, 2H), 0.97 (s, 9H).

### Example A161. 4-amino-N-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N-methylimidazo[1,5-a]quinoxaline-8-carboxamide

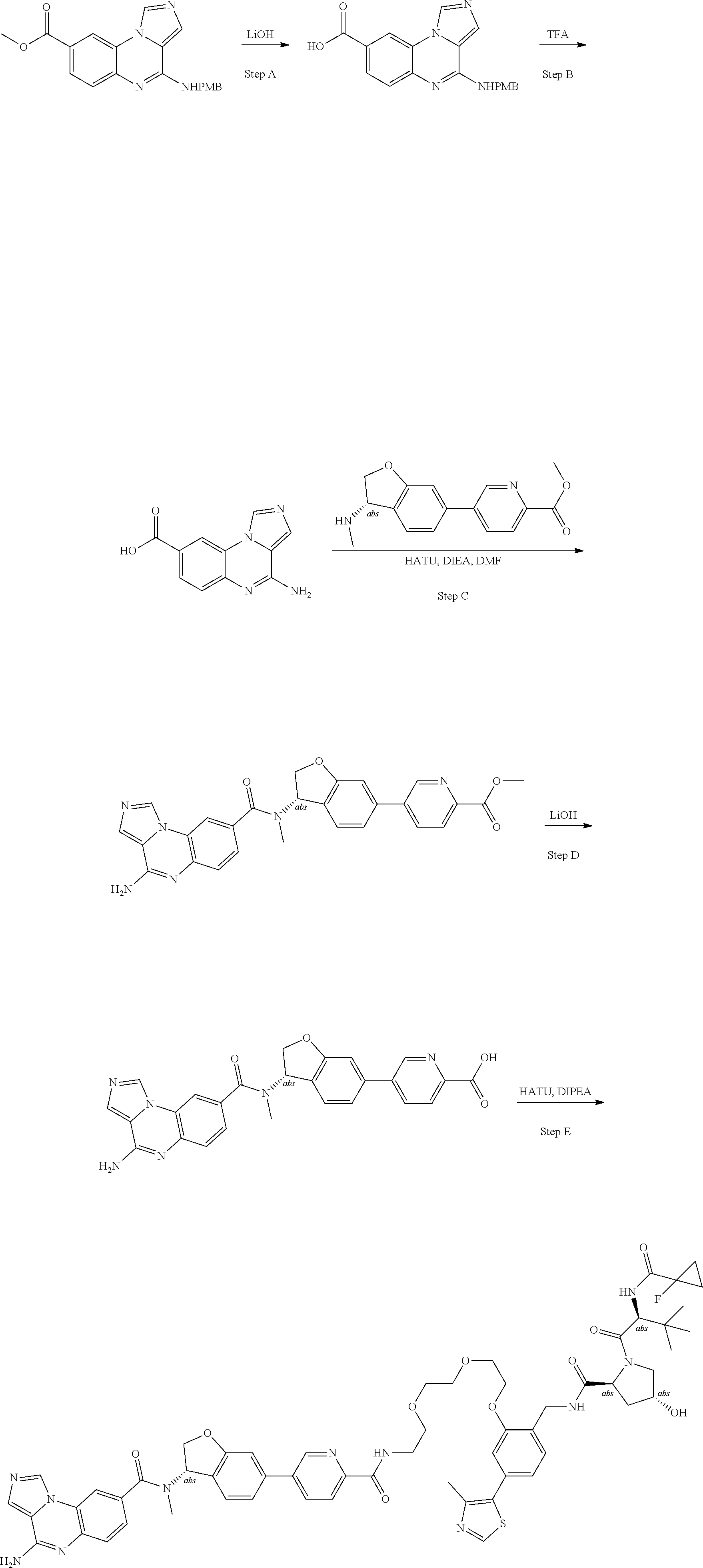

Example A161 was synthesized following the same procedures as described in Example A13 (7 mg, 5% yield over 4 steps) as a white solid. LC-MS (ESI): mass calcd, for C₅₈H₆₄FN₁₀O₁₀S, 1125.45; m/z found, 1126.6 [M+H]⁺. ¹H NMR (400 MHz, DMSO-d₆) δ 9.24 (s, 1H), 8.96-8.89 (m, 2H), 8.71 (t, J=5.8 Hz, 1H), 8.48 (t, J=5.8 Hz, 1H), 8.38 (s, 1H), 8.25 (d, J=8.4 Hz, 1H), 8.08 (d, J=8.3 Hz, 1H), 7.99 (s, 1H), 7.65-7.49 (m, 4H), 7.48-7.30 (m, 4H), 7.28 (d, J=6.5 Hz, 1H), 7.01 (s, 1H), 6.95 (d, J=7.6 Hz, 1H), 5.16 (s, 1H), 4.87-4.65 (m, 2H), 4.59 (d, J=9.1 Hz, 1H), 4.51 (t, J=8.2 Hz, 1H), 4.36-4.32 (m, 1H), 4.25 (dd, J=23.6, 5.7 Hz, 2H), 4.19-4.16 (m, 2H), 3.82-3.79 (m, 2H), 3.65-3.63 (m, 2H), 3.61-3.57 (m, 4H), 3.50-3.47 (m, 2H), 3.35 (s, 1H), 2.70 (s, 3H), 2.54-2.53 (m, 2H), 2.43 (s, 3H), 2.10-2.05 (m, 1H), 1.94-1.88 (m, 1H), 1.39-1.32 (m, 2H), 1.23-1.19 (m, 2H), 0.94 (s, 9H).

### Example A162. 2-amino-N-((R)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl)pyridin-3-yl)-2,3-dihydrobenzofuran-3-yl)-N-isopropyl-3-methylquinoline-6-carboxamide

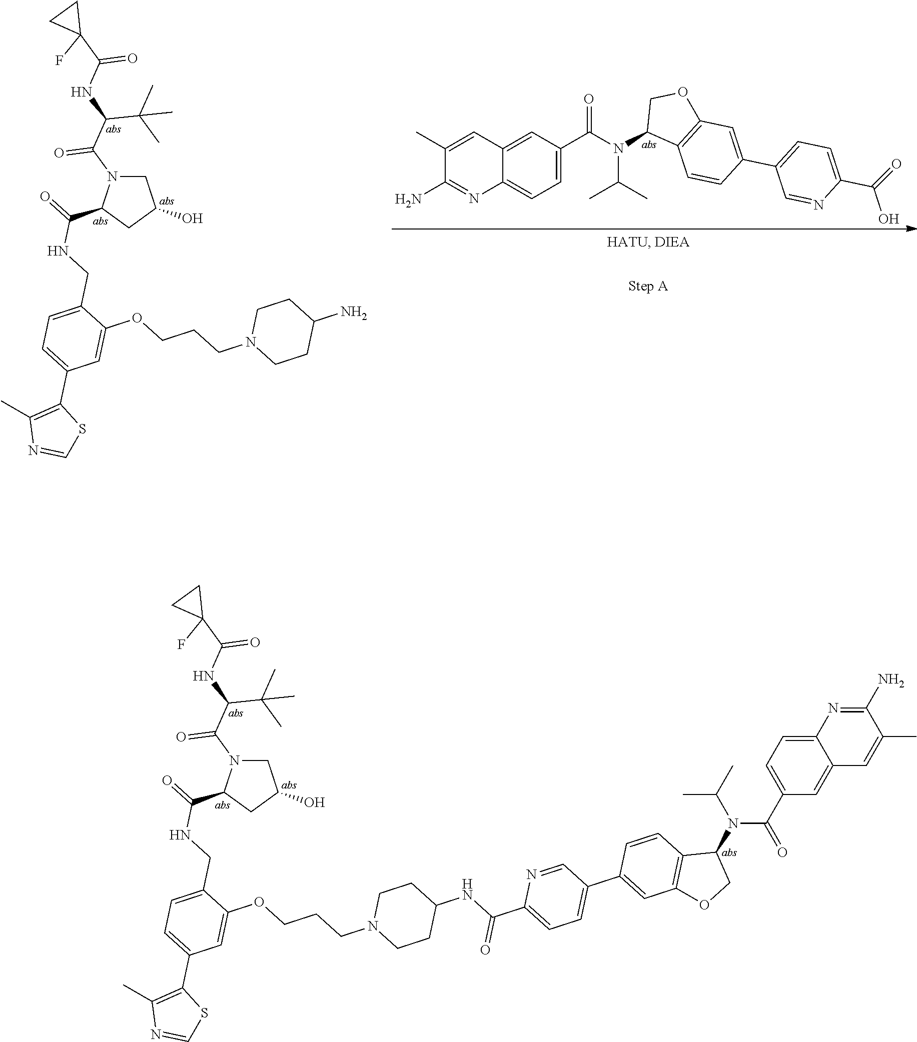

Example A162 was synthesized following the same procedures as described in Example A45 (2.9 mg, 9% yield) as a white solid. LC-MS (ESI): mass calcd, for C₆₂H₇₃FN₁₀O₈S, 1136.5/z found, 1137.4 [M+H]⁺. ¹H NMR (400 MHz, DMSO-d₆) δ 8.99 (s, 1H), 8.91 (s, 1H), 8.60 (d, J=8.3 Hz, 1H), 8.54-8.47 (m, 1H), 8.24 (d, J=7.3 Hz, 1H), 8.16 (s, 1H), 8.08 (d, J=8.2 Hz, 1H), 7.80 (s, 1H), 7.62 (s, 1H), 7.47 (d, J=8.2 Hz, 1H), 7.43 (s, 1H), 7.39 (s, 1H), 7.33-7.25 (m, 2H), 7.20 (s, 1H), 7.02 (s, 1H), 6.97 (d, J=7.8 Hz, 1H), 6.49 (s, 2H), 5.40 (s, 2H), 4.79-4.73 (m, 1H), 4.71-4.67 (m, 1H), 4.60 (d, J=9.1 Hz, 1H), 4.55-4.50 (m, 1H), 4.36 (s, 1H), 4.32-4.24 (m, 2H), 4.14-4.09 (m, 2H), 3.91-3.82 (m, 2H), 3.69-3.59 (m, 4H), 3.50-3.46 (m, 1H), 3.03-2.96 (m, 2H), 2.66-2.59 (m, 2H), 2.47 (s, 3H), 2.22 (s, 3H), 2.13-2.07 (m, 1H), 2.00-1.91 (m, 3H), 1.85-1.78 (m, 2H), 1.76-1.68 (m, 2H), 1.44-1.33 (m, 5H), 1.27-1.15 (m, 5H), 0.96 (s, 9H).

### Example A163. N1-(8-aminoimidazo[1,5-a]pyrazin-5-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) ethoxy)ethyl)carbamoyl)pyridin-3-yl)-2,3-dihydrobenzofuran-3-yl)-N2-methyloxalamide

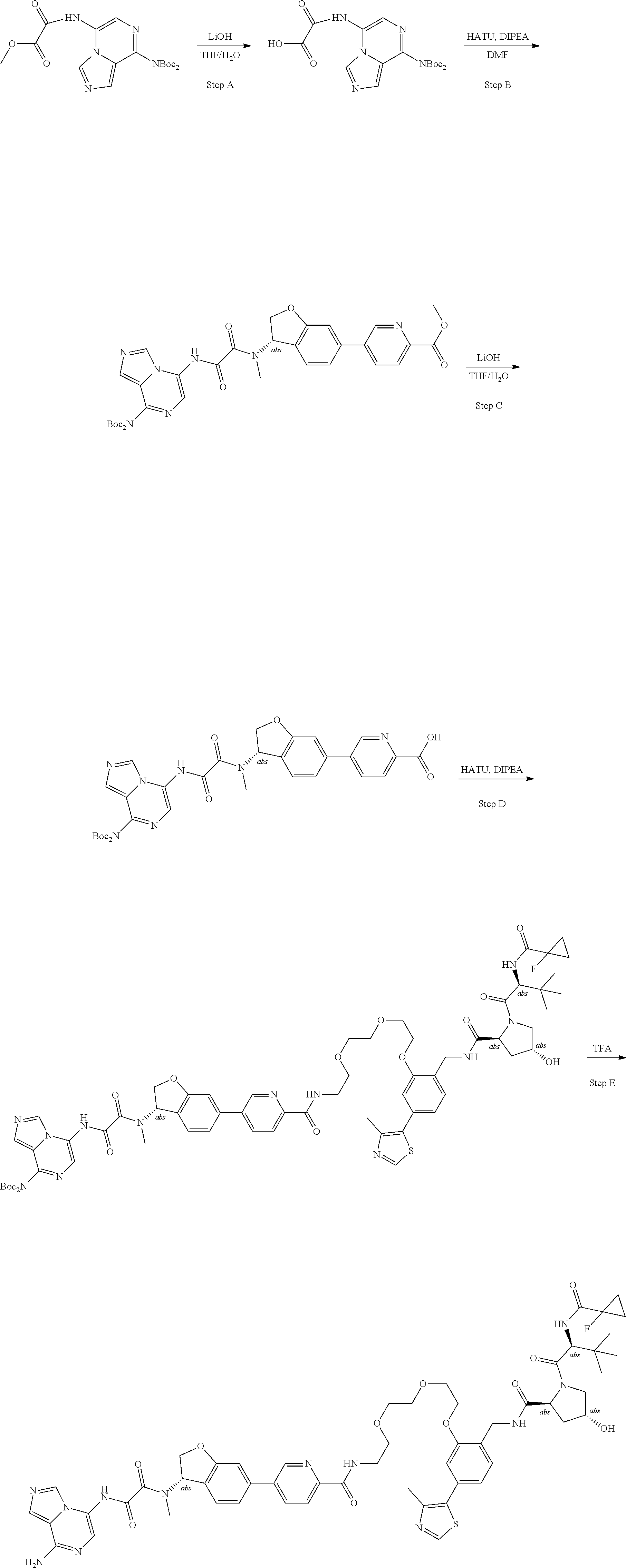

Step A: Acetic acid, 2-[[8-[bis[(1,1-dimethylethoxy)carbonyl]amino]imidazo[1,5-a]pyrazin-5-yl]amino]-2-oxo

A mixture of ethyl 2-((8-(bis(tert-butoxycarbonyl)amino) imidazo[1,5-a]pyrazin-5-yl)amino)-2-oxoacetate (40 mg, 89.0 μmol, 1.0 eq.)(WO2024/22433, 2024, A1) and lithium hydroxide (4 mg, 178 μmol, 2.0 eq.) in THF (2 mL) and H₂O (2 mL) was stirred at room temperature for 1 hour under nitrogen. The resulting solution was adjusted PH=5-6 with aq. brine (50 mL), dried over Na₂SO₄ and concentrated under reduced pressure to afford 2-((8-(bis(tert-butoxycarbonyl)amino) imidazo[1,5-a]pyrazin-5-yl)amino)-2-oxoacetic acid (20 mg, 53% yield) as a yellow solid. LC-MS (ESI): mass calcd. For C₁₅H₂₃N₅O₇, 421.16; m/z found, 422 [M+H]⁺.

Steps B-E: N1-(8-aminoimidazo[1,5-a]pyrazin-5-yl)-N2-((S)-6-(6-((2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)carbamoyl)pyridin-3-yl)-2,3-dihydrobenzofuran-3-yl)-N2-methyloxalamide

Example A163 was synthesized following the same procedures as described in Example A44 (2.6 mg, 5% yield over 4 steps) as a white solid. LC-MS (ESI): mass calcd. for C₅₅H₆₃FN₁₂O₁₁S, 1118.44; m/z found, 1119 [M+H]⁺, ¹H NMR (400 MHz, DMSO-d₆) δ 11.00 (d, J=37.8 Hz, 1H), 8.96-8.91 (m, 2H), 8.72 (d, J=5.6 Hz, 1H), 8.48 (t, J=5.9 Hz, 1H), 8.27 (d, J=8.1 Hz, 1H), 8.15-8.04 (m, 2H), 7.84 (d, J=2.4 Hz, 1H), 7.60-7.44 (m, 1H), 7.40-7.36 (m, 2H), 7.28 (d, J=9.1 Hz, 1H), 7.19 (s, 2H), 7.06-6.99 (m, 2H), 6.95 (d, J=7.7 Hz, 1H), 6.33-5.93 (m, 1H), 5.17-5.14 (m, 1H), 4.83-4.73 (m, 1H), 4.70-4.63 (m, 1H), 4.59 (d, J=8.9 Hz, 1H), 4.51 (t, J=8.1 Hz, 1H), 4.34 (s, 1H), 4.32-4.22 (m, 2H), 4.19-4.16 (m, 2H), 3.82-3.79 (m, 2H), 3.66-3.63 (m, 3H), 3.61-3.55 (m, 6H), 3.51-3.47 (m, 2H), 2.83-2.81 (m, 1H), 2.65-2.61 (m, 2H), 2.43 (s, 3H), 2.10-2.05 (m, 1H), 1.96-1.89 (m, 1H), 1.38-1.31 (m, 2H), 1.23-1.20 (m, 2H), 0.95 (s, 9H).

### Example A164. 2-amino-N-((R)-6-(6-((3-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)piperidin-1-yl) propyl)carbamoyl)pyridin-3-yl)-2,3-dihydrobenzofuran-3-yl)-N-isopropyl-3-methylquinoline-6-carboxamide

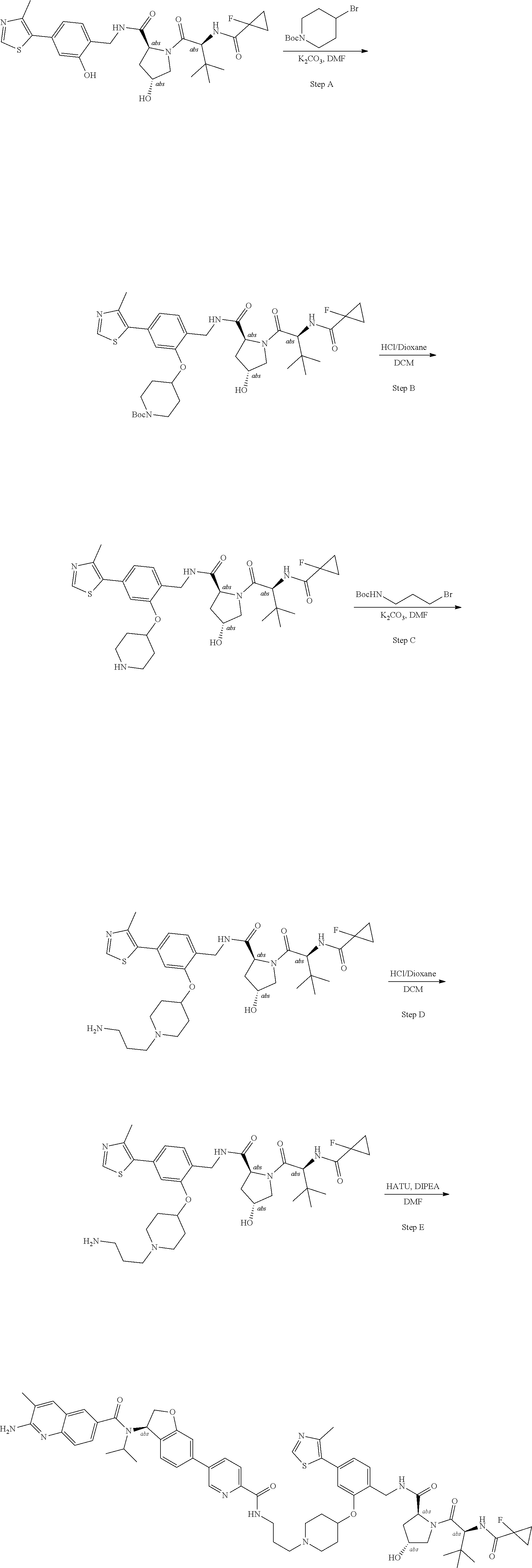

Example A164 was synthesized following the same procedures as described in Example

A23 (2.0 mg, 2% yield over 5 steps) as a white solid. LC-MS (ESI): mass calcd. for C₆₂H₇₃FN₁₀O₈S, 1136.53; m/z found, 1137.5 [M+H]⁺. ¹H NMR (400 MHz, DMSO-d₆) δ 9.19 (s, 1H), 8.96 (s, 1H), 8.90 (s, 1H), 8.50-8.43 (m, 1H), 8.23 (d, J=7.9 Hz, 1H), 8.07 (d, J=8.2 Hz, 1H), 7.78 (s, 1H), 7.61 (s, 1H), 7.48-7.45 (m, 1H), 7.42-7.37 (m, 3H), 7.31-7.26 (m, 2H), 7.20 (s, 1H), 7.06 (s, 1H), 6.92 (d, J=8.1 Hz, 1H), 6.47-6.43 (m, 2H), 5.40 (s, 1H), 5.16 (d, J=3.4 Hz, 1H), 4.76-4.68 (m, 2H), 4.59 (d, J=9.1 Hz, 2H), 4.51 (t, J=8.1 Hz, 1H), 4.34 (s, 1H), 4.33-4.21 (m, 2H), 3.66-3.58 (m, 2H), 3.44-3.35 (m, 5H), 3.44-3.35 (m, 5H), 2.54-2.52 (m, 2H), 2.44 (s, 3H), 2.43-2.41 (m, 2H), 2.21 (s, 3H), 2.08-1.98 (m, 3H), 1.95-1.90 (m, 1H), 1.84-1.73 (m, 4H), 1.42-1.34 (m, 5H), 1.24-1.18 (m, 5H), 0.95 (s, 9H).

### Example A165. 2-amino-N-((R)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-2,2-dimethylpropyl)piperidin-4-yl)carbamoyl)pyridin-3-yl)-2,3-dihydrobenzofuran-3-yl)-N-isopropyl-3-methylquinoline-6-carboxamide

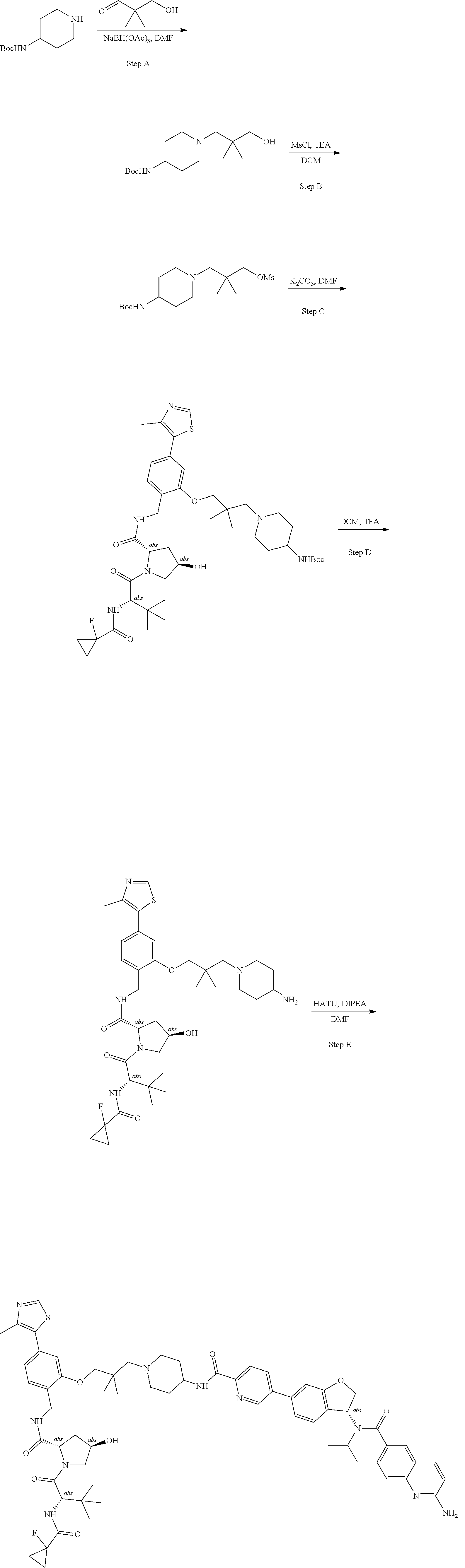

Example A165 was synthesized following the same procedures as described in Example A107 (11.0 mg, 1% yield over 5 steps) as a white solid. LC-MS (ESI): mass calcd. for C₆₄H₇₇FN₁₀O₈S, 1164.6/z found, 1165.1 [M+H]⁺, ¹H NMR (400 MHz, DMSO-d₆) δ 8.98 (s, 1H), 8.89 (s, 1H), 8.52 (t, J=5.7 Hz, 1H), 8.48 (d, J=8.3 Hz, 1H), 8.25-8.21 (m, 1H), 8.05 (d, J=8.3 Hz, 1H), 7.78 (s, 1H), 7.61 (s, 1H), 7.46 (d, J=8.4 Hz, 1H), 7.42 (d, J=7.8 Hz, 1H), 7.40-7.35 (m, 1H), 7.29 (d, J=9.3 Hz, 2H), 7.19 (s, 1H), 7.00 (d, J=5.7 Hz, 1H), 6.97-6.91 (m, 1H), 6.46 (s, 2H), 5.17 (s, 1H), 4.73 (d, J=9.2 Hz, 1H), 4.78-4.65 (m, 2H), 4.59 (d, J=9.3 Hz, 1H), 4.52 (t, J=8.2 Hz, 1H), 4.35 (s, 1H), 4.35-4.17 (m, 2H), 4.23 (dd, J=16.7, 5.4 Hz, 1H), 3.99 (s, 1H), 3.77 (s, 3H), 3.81-3.72 (m, 3H), 3.68-3.58 (m, 2H), 3.47 (s, 1H), 3.25 (d, J=3.5 Hz, 3H), 3.29-3.19 (m, 2H), 2.80-2.68 (m, 2H), 2.46 (s, 3H), 2.34 (d, J=13.9 Hz, 3H), 2.34-2.31 (m, 2H), 2.21 (s, 3H), 2.14-2.05 (m, 1H), 1.99-1.90 (m, 1H), 1.73-1.62 (m, 4H), 1.44-1.32 (m, 5H), 1.26-1.15 (m, 5H), 1.01-0.97 (m, 6H), 0.95 (s, 9H).

### Example A166. 2-amino-N-((R)-6-(6-((1′-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-[1,4′-bipiperidin]-4-yl)carbamoyl)pyridin-3-yl)-2,3-dihydrobenzofuran-3-yl)-N-isopropyl-3-methylquinoline-6-carboxamide

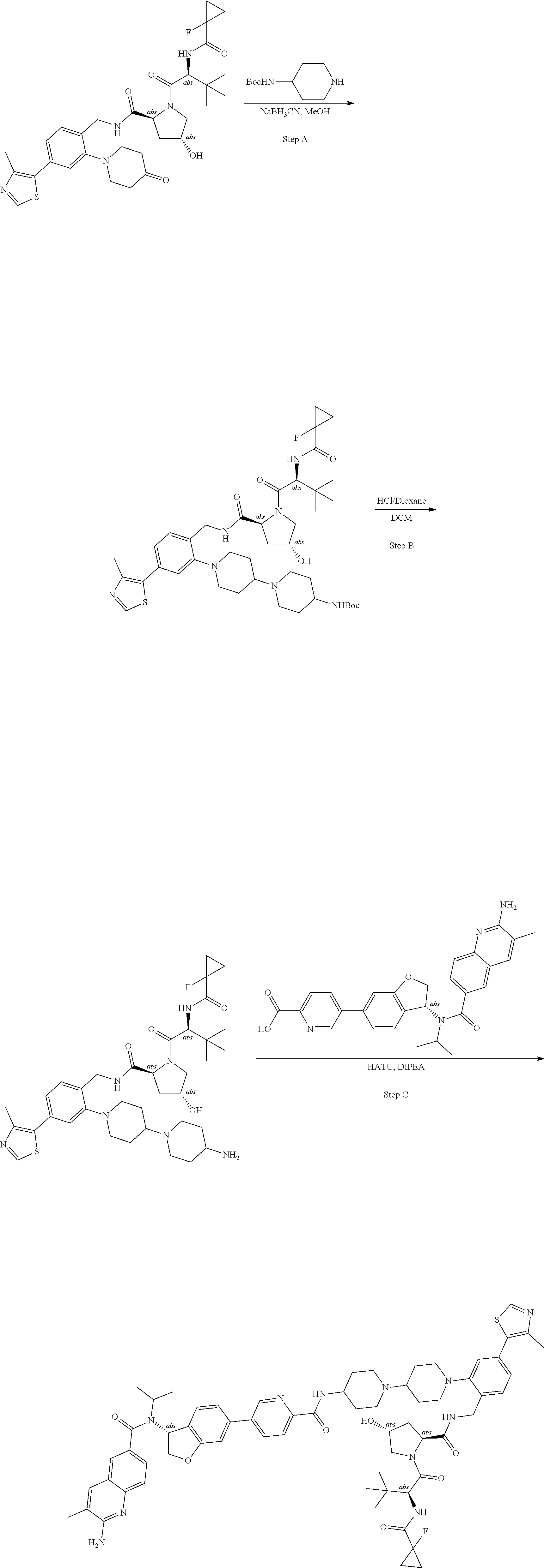

Example A166 was synthesized following the same procedures as described in Example A107 (11.8 mg, 17% yield over 3 steps) as a white solid. LC-MS (ESI): mass calcd. for C₆₄H₇₆FN₁₁O₇S, 1161.6; m/z found, 1163 [M+H]⁺. ¹H NMR (400 MHz, DMSO-d₆) δ 8.98 (s, 1H), 8.90 (s, 1H), 8.57 (d, J=8.3 Hz, 1H), 8.50 (t, 1H), 8.24 (d, J=7.2 Hz, 1H), 8.17 (s, 1H), 8.07 (d, J=8.2 Hz, 1H), 7.79 (s, 1H), 7.61 (s, 1H), 7.47-7.42 (m, 2H), 7.41-7.38 (m, 1H), 7.29-7.25 (m, 2H), 7.22-7.16 (m, 1H), 7.13-7.09 (m, 2H), 6.46 (s, 2H), 5.48-5.33 (m, 1H), 5.21-5.13 (m, 1H), 4.79-4.72 (m, 1H), 4.70-4.66 (m, 1H), 4.60 (d, J=9.2 Hz, 1H), 4.49 (t, J=8.3 Hz, 1H), 4.44-4.38 (m, 1H), 4.37-4.34 (m, 1H), 4.32-4.26 (m, 1H), 3.89-3.70 (m, 2H), 3.69-3.57 (m, 3H), 3.13-3.10 (m, 2H), 2.96-2.92 (m, 2H), 2.78-2.68 (m, 2H), 2.46 (s, 3H), 2.38-2.33 (m, 2H), 2.21 (s, 3H), 2.10-2.06 (m, 1H), 1.95-1.90 (m, 1H), 1.85-1.81 (m, 2H), 1.69-1.66 (m, 2H), 1.46-1.28 (m, 7H), 1.26-1.12 (m, 7H), 0.98 (s, 9H).

### Example A167. 2-amino-N-((S)-6-(6-(4-((9-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-3,9-diazaspiro[5.5]undecan-3-yl)methyl)piperidine-1-carbonyl)pyridin-3-yl)-2,3-dihydrobenzofuran-3-yl)-N,3-dimethylquinoline-6-carboxamide

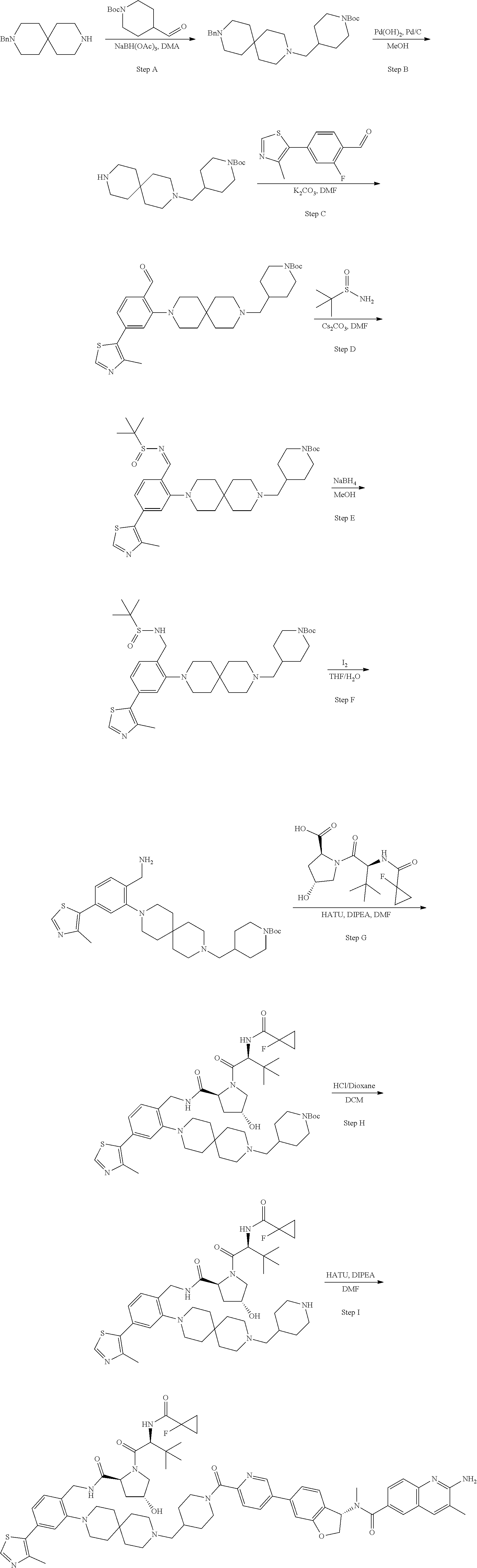

Example A167 was synthesized following the same procedures as described in Example A107 (6.2 mg, 2% yield over 9 steps) as a white solid. LC-MS (ESI): mass calcd. for C₆₇H₈₀FN₁₁O₇S, 1201.6; m/z found, 1202.9 [M+H]⁺. ¹H NMR (400 MHz, DMSO-d₆) δ 8.98 (s, 1H), 8.90-8.88 (m, 1H), 8.50-8.46 (m, 1H), 8.22-8.18 (m, 1H), 8.15 (s, 1H), 7.80 (d, J=13.1 Hz, 2H), 7.61 (d, J=8.1 Hz, 1H), 7.58-7.55 (m, 1H), 7.52-7.50 (m, 1H), 7.42 (d, J=8.0 Hz, 1H), 7.39-7.36 (m, 1H), 7.32 (s, 1H), 7.29-7.25 (m, 1H), 7.17-7.15 (m, 1H), 7.11-7.08 (m, 1H), 6.51 (s, 2H), 5.17 (s, 1H), 4.82-4.67 (m, 2H), 4.61-4.58 (m, 1H), 4.52-4.46 (m, 2H), 4.45-4.39 (m, 1H), 4.39-4.33 (m, 2H), 4.31-4.27 (m, 1H), 3.77-3.59 (m, 6H), 3.07-3.04 (m, 1H), 2.86-2.77 (m, 6H), 2.67 (s, 3H), 2.45 (s, 3H), 2.34-2.29 (m, 2H), 2.23 (s, 3H), 2.10-2.06 (m, 1H), 1.94-1.91 (m, 1H), 1.86-1.78 (m, 2H), 1.69-1.62 (m, 2H), 1.60-1.53 (m, 8H), 1.38-1.33 (m, 2H), 1.23-1.18 (m, 2H), 1.12-1.09 (m, 1H), 0.98 (s, 9H).

### Example A168. 2-amino-3-bromo-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

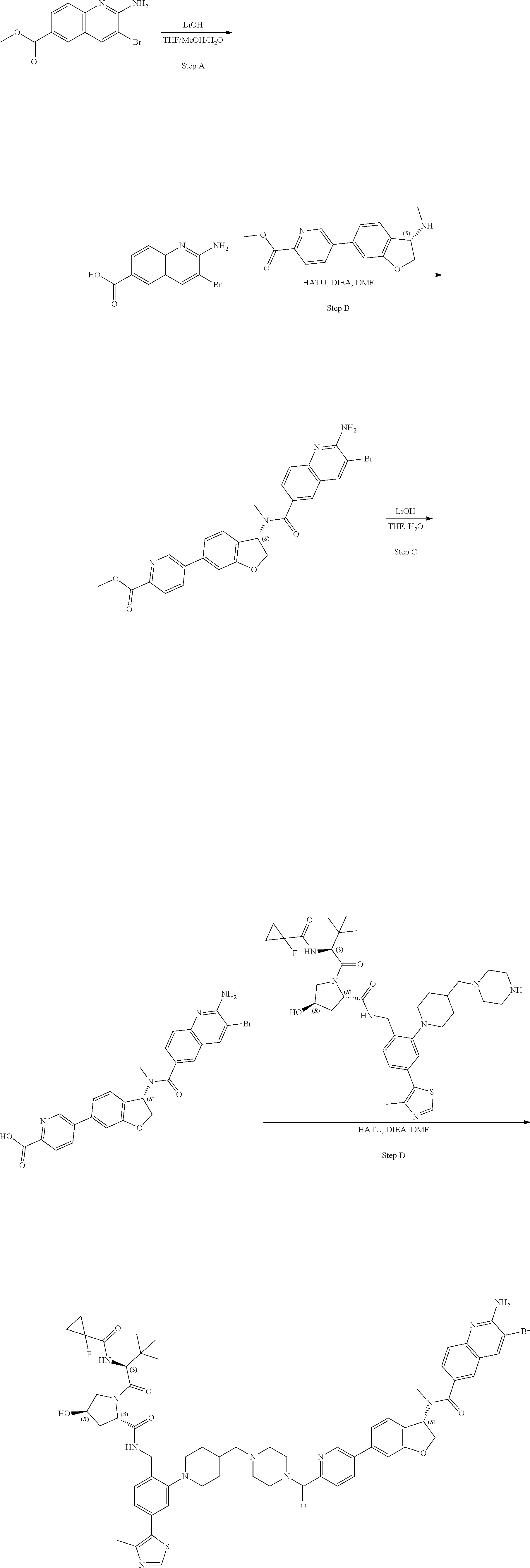

Example A168 was synthesized following the same procedures as described in Example A13 (11 mg, 15% yield over 4 steps) as a white solid. LC-MS (ESI): mass calcd. For C₆₁H₆₉BrFN₁₁O₇S, 1197.43/z found, 1198.3 [M+H]⁺. ¹H NMR (400 MHz, DMSO-d₆) δ 9.53 (s, 1H), 8.99 (s, 1H), 8.93 (s, 1H), 8.67 (s, 1H), 8.51 (s, 1H), 8.28 (d, J=8.8 Hz, 1H), 7.95 (s, 1H), 7.79-7.75 (m, 2H), 7.64 (d, J=8.5 Hz, 2H), 7.56 (d, J=7.4 Hz, 1H), 7.44 (d, J=8.4 Hz, 1H), 7.40 (d, J=7.4 Hz, 1H), 7.35 (s, 1H), 7.27 (d, J=6.8 Hz, 1H), 7.14 (s, 2H), 4.72-4.66 (m, 1H), 4.60 (d, J=9.5 Hz, 1H), 4.52-4.46 (m, 1H), 4.36 (s, 3H), 4.32-4.25 (m, 2H), 3.72-3.58 (m, 4H), 3.56-3.45 (m, 2H), 3.32 (s, 1H), 3.17 (s, 4H), 3.11 (d, J=10.7 Hz, 2H), 2.80-2.70 (m, 1H), 2.67 (s, 3H), 2.46 (s, 3H), 2.14-1.84 (m, 6H), 1.51-1.30 (m, 5H), 1.24-1.19 (m, 2H), 0.97 (d, J=8.4 Hz, 9H)

### Example A169. 2-amino-N-((S)-6-(6-(2-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,7-diazaspiro[3.5]nonane-7-carbonyl)pyridin-3-yl)-2,3-dihydrobenzofuran-3-yl)-N,3-dimethylquinoline-6-carboxamide

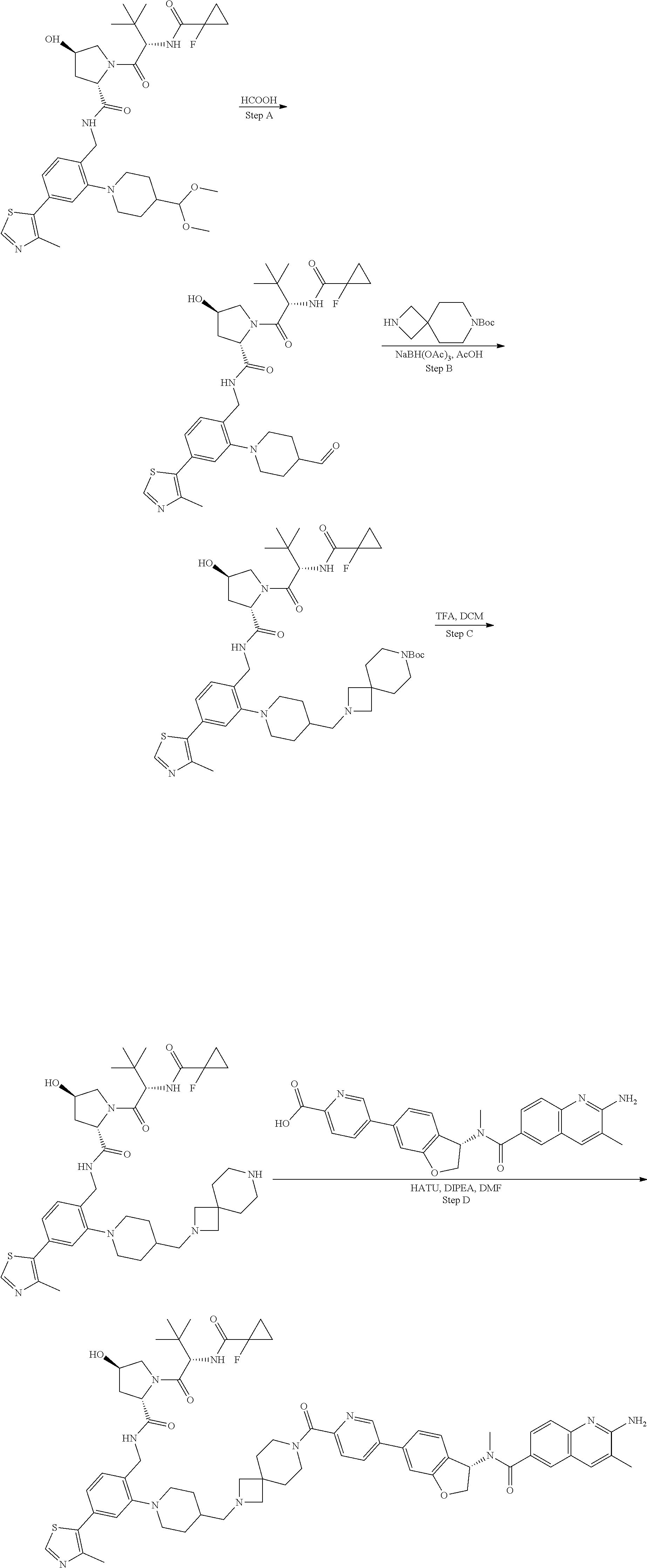

Example A169 was synthesized following the same procedures as described in Example A107 (13.6 mg, 12% yield over 4 steps) as a white solid. LC-MS (ESI): mass calcd. for C₆₅H₇₆FN₁₁O₇S, 1173.6/z found, 1175.0 [M+H]⁺. ¹H NMR (400 MHz, DMSO-d₆) δ 9.80 (s, 1H), 8.99 (s, 1H), 8.91 (s, 1H), 8.81 (s, 2H), 8.60-8.44 (m, 1H), 8.32 (s, 1H), 8.23 (d, J=9.1 Hz, 1H), 8.05 (s, 1H), 7.91-7.83 (m, 1H), 7.77 (s, 1H), 7.64 (d, J=8.2 Hz, 1H), 7.56 (d, J=7.6 Hz, 1H), 7.50-7.22 (m, 4H), 7.13 (d, J=6.3 Hz, 2H), 4.86-4.81 (m, 1H), 4.71-4.69 (m, 1H), 4.61-4.59 (m, 1H), 4.50-4.48 (m, 1H), 4.40-4.31 (m, 4H), 4.15-4.07 (m, 2H), 4.00-3.91 (m, 2H), 3.71-3.54 (m, 4H), 3.49-3.43 (m, 1H), 3.40-3.32 (m, 1H), 3.29-3.18 (m, 2H), 3.13-3.04 (m, 2H), 2.74-2.60 (m, 5H), 2.46 (s, 3H), 2.33 (s, 3H), 2.15-2.04 (m, 1H), 2.00-1.63 (m, 8H), 1.50-1.30 (m, 4H), 1.26-1.17 (m, 2H), 0.98 (s, 9H).

### Example A170. 2-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-3-(oxetan-3-yl)quinoline-6-carboxamide

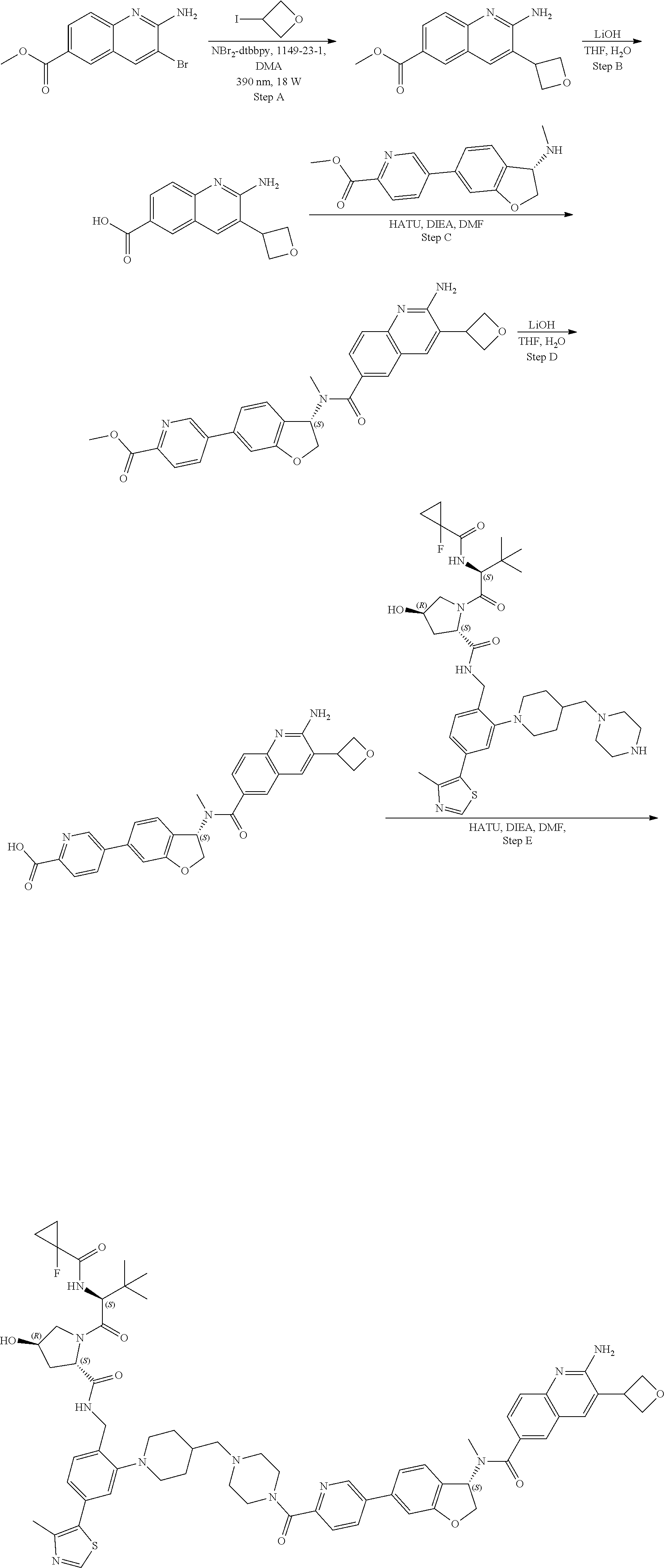

Step A: methyl 2-amino-3-(oxetan-3-yl)quinoline-6-carboxylate

To a mixture of methyl 2-amino-3-bromoquinoline-6-carboxylate (50 mg, 178 μmol, 1.0 eq.) in 1,4-Dioxane (2 mL) was added 3-iodooxetane (42 mg, 231 μmol, 1.3 eq.), TEA (54 mg, 534 μmol, 3.0 eq.), 4CzIPN (14 mg, 17.8 μmol, 0.1 eq.) and Nickel, [4,4′-bis(1,1-dimethylethyl)-2,2′-bipyridine-kappan 1, kappan1′]dibromo-(sp-4-2)-(9 mg, 17.8 μmol, 0.1 eq.). The mixture was stirred at 30° C. for 16 hours under 390 nm. The mixture was diluted with water (50 mL), extracted with EtOAc (50 mL×3). The combined organic layers dried over anhydrous Na₂SO₄, filtered and concentrated under reduced pressure to dryness. The residue was purified by column chromatography (silica gel, 0% EtOAc in PE~50% EtOAc in PE) to give the methyl 2-amino-3-(oxetan-3-yl)quinoline-6-carboxylate (20 mg, 43% yield) as yellow solid. LC-MS (ESI): mass calcd. For C₁₄H₁₄N₂O₃, 258.10; m/z found, 258.7 [M+H]⁺.

Steps B-E: 2-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)~2,3-dihydrobenzofuran-3-yl)-N-methyl-3-(oxetan-3-yl)quinoline-6-carboxamide

Example A170 was synthesized following the same procedures as described in Example A23 (3.0 mg, 6% yield over 5 steps) as a white solid. LC-MS (ESI): mass calcd. For C₆₄H₇₄FN₁₁O₈S, 1175.54/z found, 1177.0 [M+H]⁺. ¹H NMR (400 MHz, DMSO-d₆) δ 9.60 (s, 1H), 8.99 (s, 1H), 8.94 (s, 1H), 8.71-8.59 (m, 2H), 8.53-8.49 (m, 1H), 8.28 (d, J=8.5 Hz, 1H), 8.17 (s, 1H), 7.91 (d, J=9.0 Hz, 1H), 7.81-7.74 (m, 2H), 7.57 (d, J=7.8 Hz, 1H), 7.46-7.39 (m, 2H), 7.36 (s, 1H), 7.27 (d, J=7.3 Hz, 1H), 7.15-7.11 (m, 2H), 5.05-5.00 (m, 2H), 4.79-4.66 (m, 4H), 4.60 (d, J=9.0 Hz, 2H), 4.51-4.47 (m, 1H), 4.44-4.40 (m, 1H), 4.39-4.19 (m, 5H), 3.51-3.27 (m, 6H), 3.21-3.05 (m, 7H), 2.75-2.66 (m, 5H), 2.46 (s, 3H), 2.12-2.06 (m, 1H), 2.01-1.85 (m, 4H), 1.48-1.33 (m, 4H), 1.24-1.19 (m, 2H), 0.96 (s, 9H).

### Example A171. 2-amino-3-cyclopropyl-N-((S)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl) pyridin-3-yl)-2,3-dihydrobenzofuran-3-yl)-N-methylquinoline-6-carboxamide

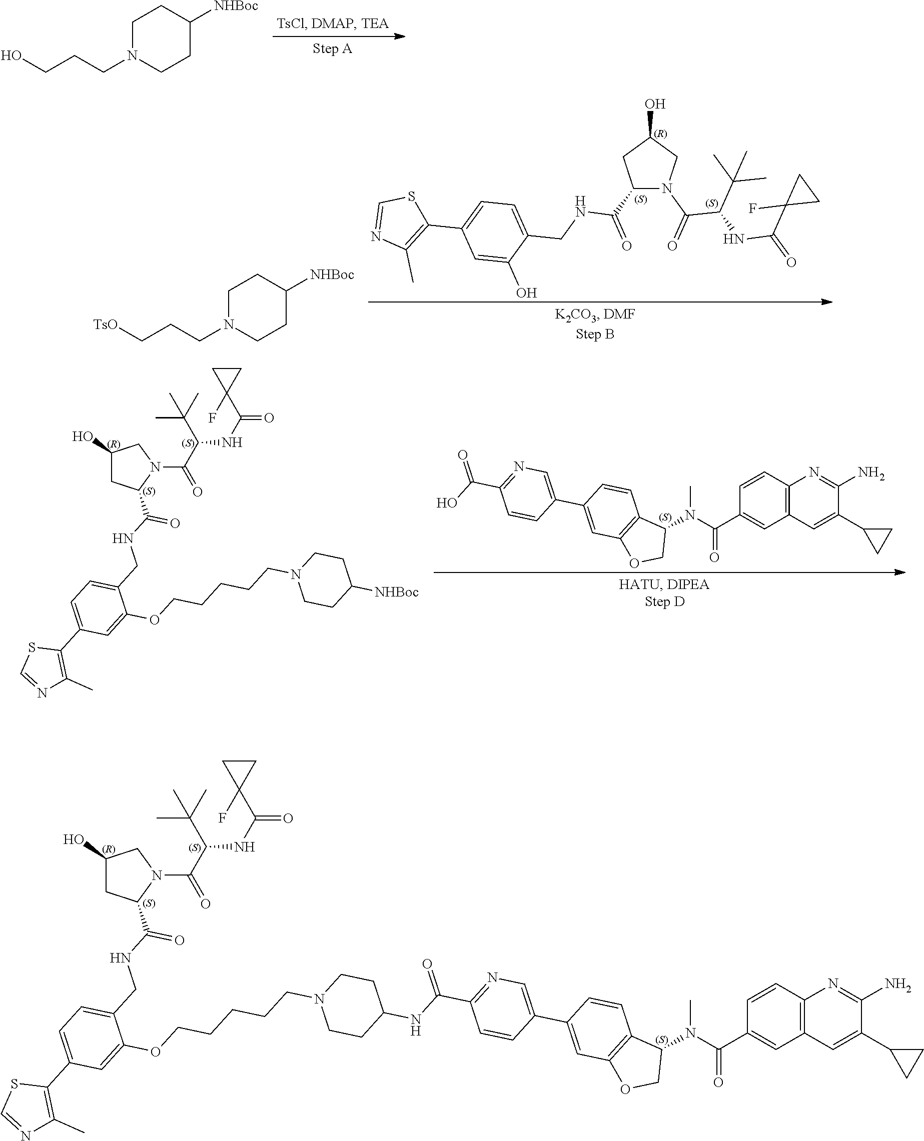

Example A171 was synthesized following the same procedures as described in Example A23 (10.5 mg, 2% yield over 4 steps) as a white solid. LC-MS (ESI): mass calcd. for C₆₂H₇₁FN₁₀O₈S, 1134.5/z found, 1136.2 [M+H]⁺. ¹H NMR (400 MHz, DMSO-d₆) δ 9.35 (d, J=58.4 Hz, 1H), 9.02-8.94 (m, 3H), 8.93-8.67 (m, 2H), 8.58-8.48 (m, 1H), 8.33-8.26 (m, 1H), 8.21 (s, 1H), 8.11 (d, J=8.2 Hz, 1H), 8.04 (s, 1H), 7.86 (d, J=8.6 Hz, 1H), 7.76 (s, 1H), 7.56 (d, J=7.7 Hz, 1H), 7.47-7.34 (m, 3H), 7.32-7.26 (m, 1H), 7.04-6.99 (m, 2H), 4.72-4.67 (m, 1H), 4.60 (d, J=9.1 Hz, 1H), 4.55-4.50 (m, 1H), 4.38-4.35 (m, 1H), 4.32-4.29 (m, 2H), 4.19-4.10 (m, 4H), 3.67-3.60 (m, 4H), 3.45-3.39 (m, 1H), 3.34-3.25 (m, 2H), 3.23-3.10 (m, 2H), 2.66 (s, 3H), 2.47 (s, 3H), 2.22-2.15 (m, 2H), 2.12-2.00 (m, 3H), 1.99-1.87 (m, 4H), 1.41-1.32 (m, 2H), 1.24-1.19 (m, 2H), 1.12-1.04 (m, 2H), 0.96 (s, 9H), 0.85-0.78 (m, 2H).

### Example A172. 2-amino-3-cyclopropyl-N-((S)-6-(6-(9-(2-(2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)-3,9-diazaspiro[5.5]undecane-3-carbonyl)pyridin-3-yl)-2,3-dihydrobenzofuran-3-yl)-N-methylquinoline-6-carboxamide

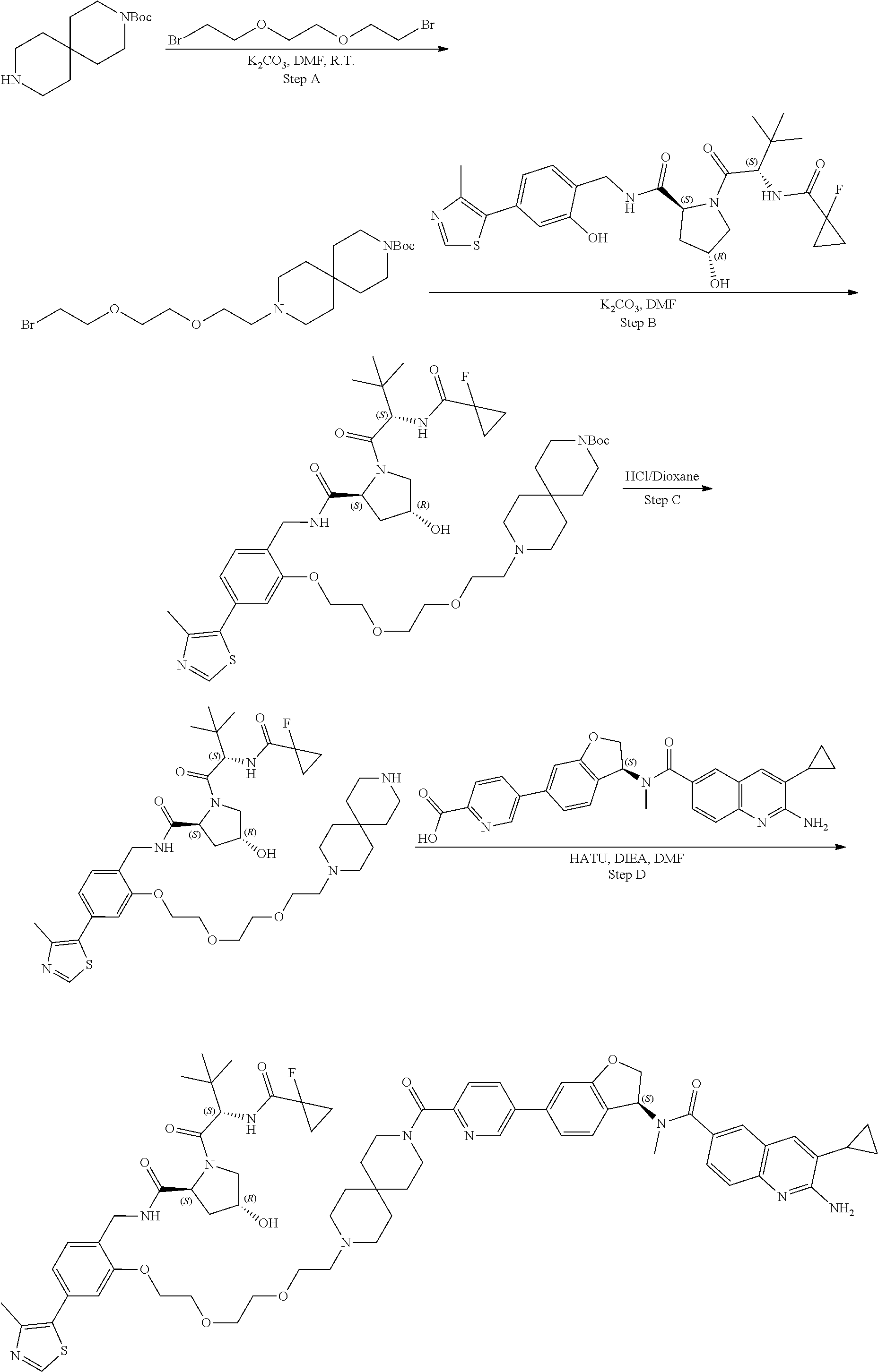

Example A172 was synthesized following the same procedures as described in Example A17 (24.9 mg, 5% yield over 4 steps) as a white solid. LC-MS (ESI): mass calcd. for C₆₉H₈₃FN₁₀O₁₀S, 1262.60; m/z found, 632.7 [1/2 M+H]⁺. ¹H NMR (400 MHz, DMSO-d₆) δ 9.27 (s, 1H), 9.03-8.84 (m, 3H), 8.56 (t, J=5.8 Hz, 1H), 8.20 (d, J=8.1 Hz, 2H), 8.05 (s, 1H), 7.87 (d, J=9.1 Hz, 1H), 7.80-7.69 (m, 1H), 7.62-7.51 (m, 2H), 7.46-7.36 (m, 2H), 7.36-7.23 (m, 2H), 7.06-7.01 (m, 1H), 7.00-6.91 (m, 1H), 4.89-4.72 (m, 1H), 4.70-4.66 (m, 1H), 4.60 (d, J=8.2 Hz, 1H), 4.56-4.50 (m, 1H), 4.38-4.28 (m, 2H), 4.27-4.21 (m, 1H), 4.20-4.16 (m, 2H), 3.84-3.75 (m, 6H), 3.67 (s, 6H), 3.43-3.22 (m, 7H), 3.13-2.97 (m, 2H), 2.66 (s, 3H), 2.46 (s, 3H), 2.15-2.05 (m, 1H), 1.96-1.87 (m, 2H), 1.86-1.74 (m, 2H), 1.65-1.47 (m, 4H), 1.40-1.20 (m, 6H), 1.12-1.05 (m, 2H), 0.95 (d, J=5.6 Hz, 9H), 0.85-0.78 (m, 2H).

### Example A173. 2-amino-3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

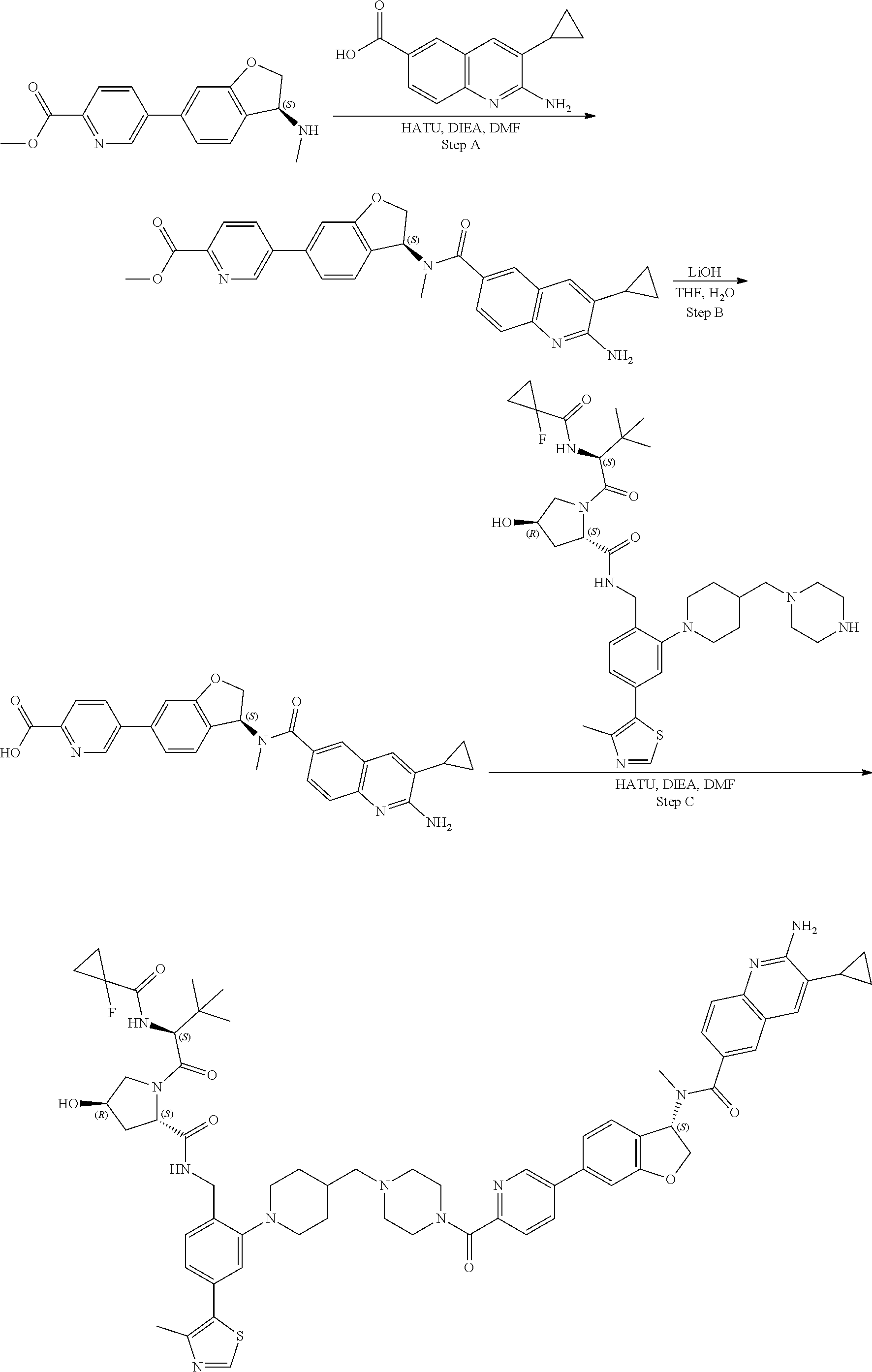

Example A173 was synthesized following the same procedures as described in Example A51 (3.5 mg, 7% yield over 3 steps) as a white solid. LC-MS (ESI): mass calcd. for C₆₄H₇₄FN₁₁O₇S, 1159.55; m/z found, 580.9 [1/2 M+H]⁺. ¹H NMR (400 MHz, DMSO-d₆) δ 9.52 (s, 1H), 8.99 (s, 1H), 8.93 (s, 1H), 8.86-8.72 (m, 2H), 8.52 (s, 1H), 8.28 (d, J=8.4 Hz, 1H), 8.22 (s, 1H), 8.05 (s, 1H), 7.87 (d, J=8.3 Hz, 1H), 7.78 (d, J=8.0 Hz, 2H), 7.56 (d, J=7.6 Hz, 1H), 7.47-7.39 (m, 2H), 7.36 (s, 1H), 7.27 (d, J=7.1 Hz, 1H), 7.14 (s, 2H), 4.70-4.67 (m, 1H), 4.60 (d, J=9.5 Hz, 2H), 4.52-4.47 (m, 2H), 4.39-4.34 (m, 4H), 3.70-3.57 (m, 9H), 3.36-3.29 (m, 1H), 3.21-3.09 (m, 6H), 2.78-2.69 (m, 2H), 2.67 (s, 3H), 2.46 (s, 3H), 2.13-2.07 (m, 1H), 2.03-1.96 (m, 1H), 1.93-1.83 (m, 4H), 1.49-1.41 (m, 2H), 1.40-1.32 (m, 2H), 1.24-1.20 (m, 2H), 1.08 (d, J=8.2 Hz, 2H), 0.98 (s, 9H), 0.82 (s, 2H).

### Example A174. 2-amino-3-cyano-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

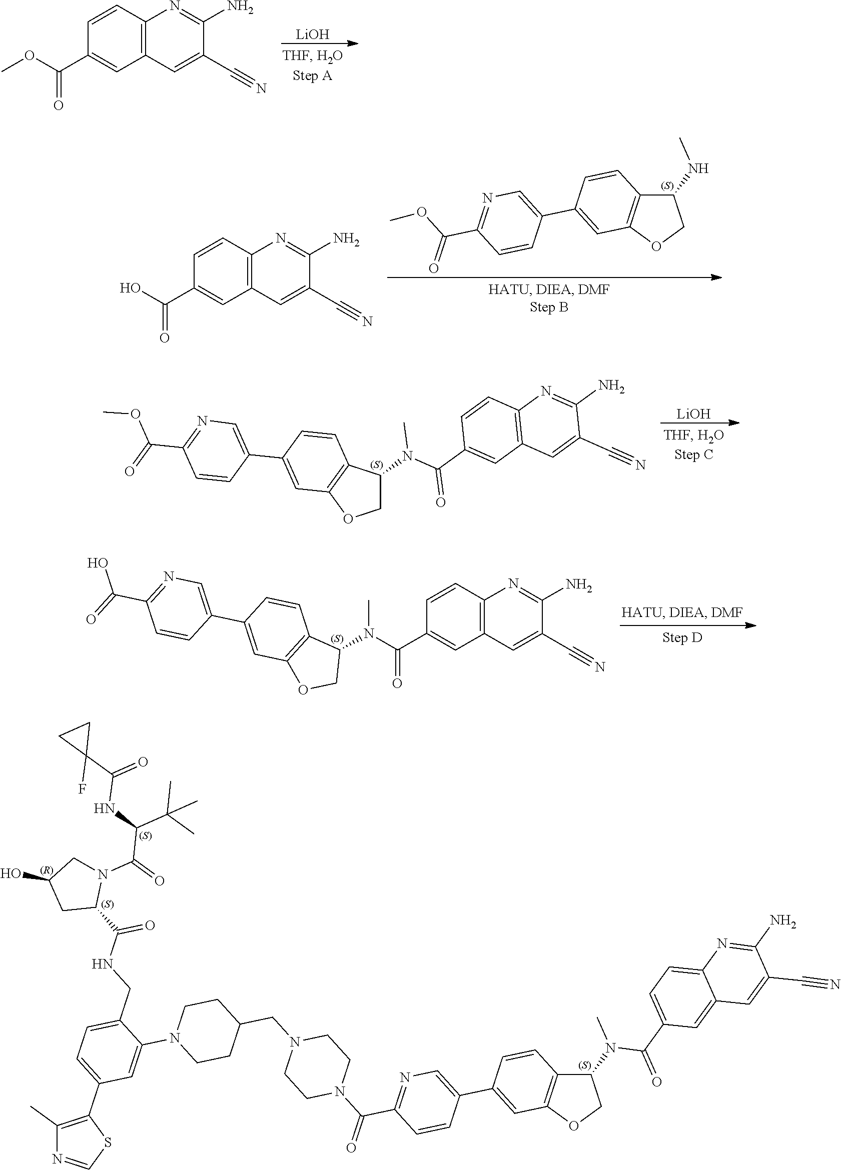

Example A174 was synthesized following the same procedures as described in Example A51 (3.5 mg, 5% yield over 3 steps) as a white solid. LC-MS (ESI): mass calcd. for C₆₂H₆₉FN₁₂O₇S, 1144.51; m/z found, 1145 [M+H]⁺. ¹H NMR (400 MHz, DMSO-d₆) δ 8.98 (s, 1H), 8.91 (s, 1H), 8.77 (s, 1H), 8.49 (t, J=5.9 Hz, 1H), 8.25-8.19 (m, 1H), 7.95 (s, 1H), 7.79 (dd, J=8.7, 1.8 Hz, 1H), 7.66 (d, J=8.2 Hz, 1H), 7.56 (t, J=8.0 Hz, 2H), 7.41 (dd, J=14.7, 7.7 Hz, 2H), 7.33 (s, 1H), 7.28 (d, J=6.7 Hz, 1H), 7.19 (s, 2H), 7.10 (d, J=9.7 Hz, 2H), 5.17 (s, 1H), 4.81-4.66 (m, 2H), 4.60 (d, J=9.3 Hz, 1H), 4.50-4.45 (m, 1H), 4.41-4.27 (m, 3H), 3.71-3.59 (m, 4H), 3.50-3.46 (m, 2H), 3.09-3.02 (m, 2H), 2.74-2.60 (m, 6H), 2.47-2.44 (m, 5H), 2.40-2.35 (m, 2H), 2.27-2.22 (m, 2H), 2.11-2.05 (m, 1H), 1.96-1.89 (m, 1H), 1.85-1.77 (m, 2H), 1.72-1.64 (m, 1H), 1.41-1.29 (m, 4H), 1.24-1.20 (m, 2H), 0.97 (s, 9H).

### Example A175. 4-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,1,3-trimethyl-1H-pyrazolo[4,3-c]quinoline-8-carboxamide

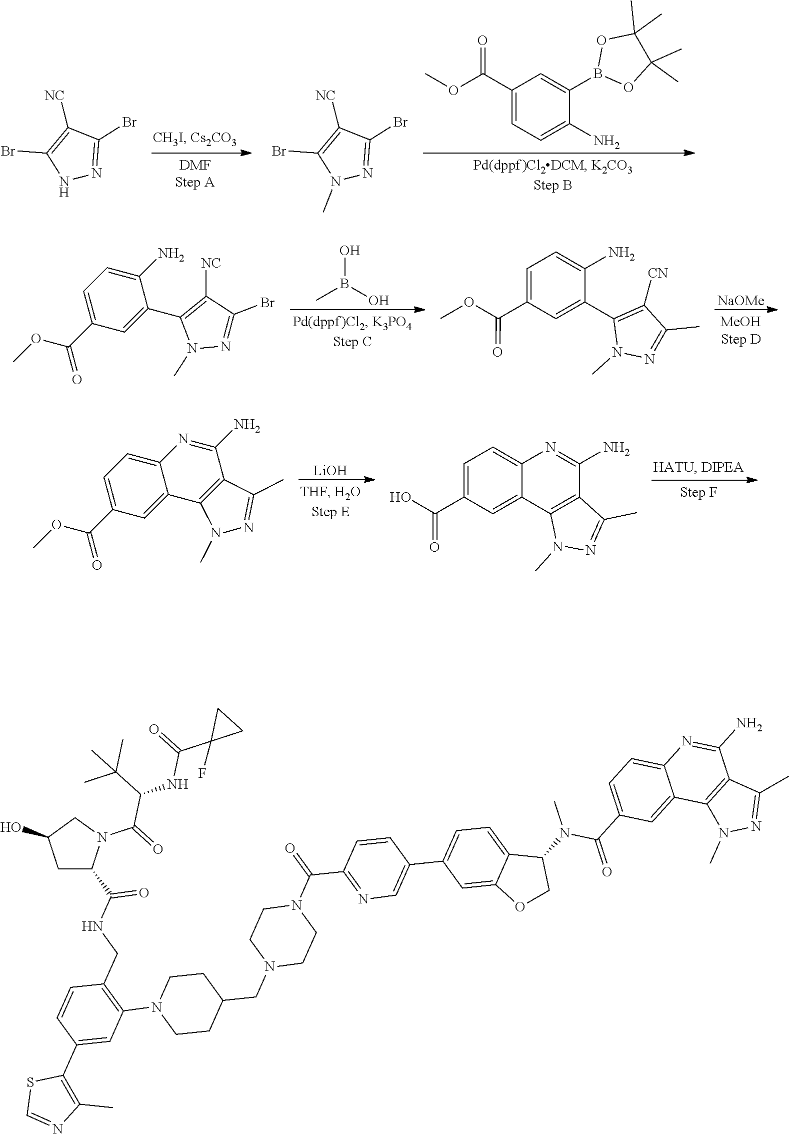

Example A175 was synthesized following the same procedures as described in Example A41 (5.3 mg, 1.7% yield over 6 steps) as a white solid. LC-MS (ESI): mass calcd. for C₆₄H₇₄FN₁₃O₇S, 1187.55 m/z found, 1188.1 [M+H]⁺. ¹H NMR (400 MHz, DMSO-d₆) δ 9.56 (s, 1H), 8.99 (s, 1H), 8.94 (s, 1H), 8.79 (s, 1H), 8.52 (s, 1H), 8.43 (s, 1H), 8.28 (d, J=6.7 Hz, 1H), 7.93 (d, J=9.2 Hz, 1H), 7.87 (s, 1H), 7.78 (d, J=8.2 Hz, 1H), 7.61 (s, 1H), 7.44 (d, J=8.3 Hz, 2H), 7.36 (s, 1H), 7.27 (d, J=6.7 Hz, 1H), 7.13 (d, J=4.1 Hz, 2H), 4.75 (s, 2H), 4.60 (d, J=9.4 Hz, 2H), 4.49 (t, J=8.2 Hz, 1H), 4.43 (s, 3H), 4.39-4.33 (m, 3H), 4.27-4.18 (m, 1H), 3.70-3.61 (m, 7H), 3.35-3.30 (m, 1H), 3.19-3.07 (m, 6H), 2.77-2.69 (m, 4H), 2.67 (s, 3H), 2.46 (s, 3H), 2.13-2.06 (m, 1H), 2.03-1.96 (m, 1H), 1.95-1.91 (m, 1H), 1.90-1.82 (m, 2H), 1.52-1.43 (m, 2H), 1.41-1.37 (m, 1H), 1.37-1.33 (m, 1H), 1.22 (d, J=8.6 Hz, 2H), 0.98 (s, 9H).

### Example A176. 2-amino-3-cyclopropyl-N-((S)-6-(6-(((1S,4S)-4-(ethyl(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino)cyclohexyl)carbamoyl)

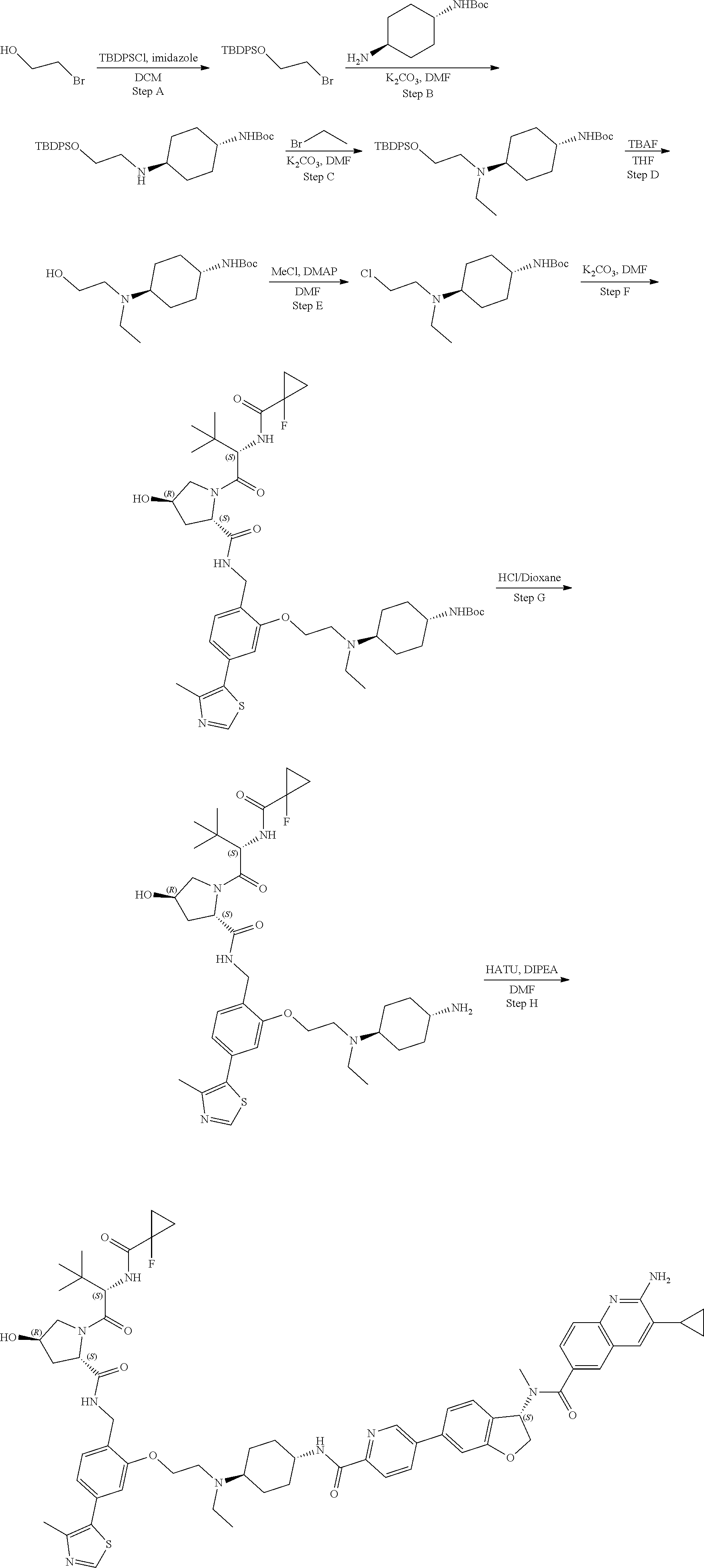

Step A: (2-bromoethoxy)(tert-butyl)diphenylsilane

To a solution of 2-bromoethan-1-ol (5 g, 40 mmol, 1.0 eq.) in DCM (50 mL) was added tert-Butyldiphenylchlorosilane (11.2 mL, 44 mmol, 1.1 eq.) and Imidazole (5.45 g, 80 mmol, 2.0 eq.) under N₂. The mixture was stirred at room temperature for 16 hours to give yellow solution. The mixture was filtered and concentrated. The residue was purified by column chromatography (silica gel, 0% EtOAc in PE~20% EtOAc in PE) to afford (2-bromoethoxy)(tert-butyl)diphenylsilane (13.5 g, 88% yield) as a yellow oil. ¹HNMR (400 MHz, DMSO-d₆) δ 7.76-7.53 (m, 4H), 7.61-7.35 (m, 6H), 3.93 (t, J=5.6 Hz, 2H), 3.60 (t, J=5.6 Hz, 2H), 1.02 (s, 9H).

Step B: tert-butyl ((1r,4r)-4-((2-((tert-butyldiphenylsilyl)oxy)ethyl)amino)cyclohexyl)carbamate

To a solution of tert-butyl ((1r,4r)-4-aminocyclohexyl)carbamate (2 g, 9.3 mmol, 1.0 eq.) in MeCN (50 mL) was added K₂CO₃ (2.58 g, 18.7 mmol, 2.0 eq.) and (2-bromoethoxy)(tert-butyl)diphenylsilane (2.7 g, 7.5 mmol, 0.8 eq.) under N₂. The mixture was stirred at 80° C. for 16 hours to give yellow solution. The reaction mixture was quenched with water, and then extracted with EtOAc (100 mL×2). The combined organic layers were washed with brine three times, dried over anhydrous Na₂SO₄ and concentrated. The residue was purified by column chromatography (silica gel, 0% EtOAc in PE~100% EtOAc in PE) to afford tert-butyl ((1r,4r)-4-((2-((tert-butyldiphenylsilyl)oxy)ethyl)amino)cyclohexyl)carbamate (2.6 g, 53% yield) as a yellow oil. LC-MS (ESI): mass calcd. For C29H44N₂O₃Si, 496.3; m/z found, 441.3 [M+H−56]⁺.

Step C: tert-butyl ((1r, 4r)-4-((2-((tert-butyldiphenylsilyl)oxy)ethyl)(ethyl)amino)cyclohexyl)carbamate

To a solution of tert-butyl ((1r,4r)-4-((2-((tert-butyldiphenylsilyl)oxy)ethyl)amino)cyclohexyl)carbamate (500 mg, 1.01 mmol, 1.0 eq.) in MeCN (10 mL) was added K₂CO₃ (417 mg, 3.02 mmol, 3.0 eq.) and iodoethane (471 mg, 3.02 mmol, 3.0 eq.) under N₂. The mixture was stirred at 55° C. for 16 hours to give yellow solution. The reaction mixture was quenched with water (50 mL), and then extracted with EtOAc (100 mL×2). The combined organic layers were washed with brine three times, dried over anhydrous Na₂SO₄ and concentrated. The residue was purified by column chromatography (silica gel, 0% EtOAc in PE~50% EtOAc in PE) to afford tert-butyl ((1r,4r)-4-((2-((tert-butyldiphenylsilyl)oxy)ethyl)(ethyl)amino)cyclohexyl)carbamate (300 mg, 54% yield) as a yellow solid. LC-MS (ESI): mass calcd. For C31H48N₂O₃Si, 524.3; m/z found, 469.3 [M+H−56]*.

Step D: tert-butyl ((1r,4r)-4-(ethyl(2-hydroxyethyl)amino)cyclohexyl)carbamate

To a solution of tert-butyl ((1r,4r)-4-((2-((tert-butyldiphenylsilyl)oxy)ethyl)(ethyl)amino)cyclohexyl)carbamate (100 mg, 191 μmol, 1.0 eq.) in THF (5 mL) was added TBAF (49.8 mg, 191 μmol, 1.0 eq.) under N₂. The mixture was stirred at room temperature for 1 hour to give yellow solution. The reaction mixture was quenched with water, and then extracted with EtOAc (100 mL×2). The combined organic layers were washed with brine three times, dried over anhydrous Na₂SO₄ and concentrated. The residue was purified by column chromatography (silica gel, 0% EtOAc in PE~100% EtOAc in PE) to afford to afford tert-butyl ((1r,4r)-4-(ethyl(2-hydroxyethyl)amino)cyclohexyl)carbamate (200 mg, 58% yield) as a yellow solid. LC-MS (ESI): mass calcd. For C₁₅H₃₀N₂O₃, 286.2; m/z found, 231.2 [M+H−56]⁺.

Step E: tert-butyl ((1r,4r)-4-((2-chloroethyl)(ethyl)amino)cyclohexyl)carbamate

To a solution of tert-butyl ((1r,4r)-4-(ethyl(2-hydroxyethyl)amino)cyclohexyl)carbamate (50 mg, 175 μmol, 1.0 eq) in DCM (6 mL) was added DMAP (4.27 mg, 34.9 μmol, 0.2 eq.), TEA (73 μL, 524 μmol, 3.0 eq.) and Ms-Cl (27.2 μL, 349 μmol, 2.0 eq.) under N₂. The mixture was stirred at room temperature for 2 hours to give yellow solution. The reaction mixture was quenched with water, and then extracted with EtOAc (50 mL×2). The combined organic layers were washed with brine three times, dried over anhydrous Na₂SO₄ and concentrated to afford tert-butyl ((1r,4r)-4-((2-chloroethyl)(ethyl)amino)cyclohexyl)carbamate (50 mg, 94% yield) as a yellow solid. LC-MS (ESI): mass calcd. For C₁₅H₂₉ClN₂O₂, 304.2; m/z found, 249.2 [M+H−56]⁺.

Steps F-H: 2-amino-3-cyclopropyl-N-((S)-6-(6-(((1S,4S)-4-(ethyl(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino)cyclohexyl)carbamoyl) pyridin-3-yl)-2,3-dihydrobenzofuran-3-yl)-N-methylquinoline-6-carboxamide

Example A176 was synthesized following the same procedures as described in Example A23 (12.4 mg, 10% yield over 3 steps) as a white solid. LC-MS (ESI): mass calcd. for C₆₄H₇₅FN₁₀O₈S, 1162.49; m/z found, 1163.5 [M+H]⁺. ¹H NMR (400 MHz, DMSO-d₆) δ 9.10 (s, 1H), 9.02 (s, 1H), 8.93 (s, 1H), 8.68-8.47 (m, 2H), 8.30-8.27 (m, 1H), 8.21 (s, 1H), 8.09 (d, J=8.3 Hz, 1H), 8.05 (s, 1H), 7.87 (d, J=8.5 Hz, 1H), 7.76 (s, 1H), 7.56 (d, J=7.9 Hz, 1H), 7.47 (d, J=7.7 Hz, 1H), 7.43-7.33 (m, 2H), 7.27 (d, J=7.1 Hz, 1H), 7.09-7.04 (m, 2H), 4.82 (s, 1H), 4.69 (d, J=6.7 Hz, 2H), 4.59 (d, J=9.3 Hz, 1H), 4.53 (t, J=8.3 Hz, 1H), 4.40-4.33 (m, 6H), 3.92-3.80 (m, 2H), 3.67-3.55 (m, 3H), 3.45-3.30 (m, 3H), 2.66 (s, 3H), 2.48 (s, 3H), 2.22-2.05 (m, 3H), 2.03-1.84 (m, 4H), 1.80-1.70 (m, 2H), 1.68-1.54 (m, 2H), 1.43-1.26 (m, 5H), 1.26-1.12 (m, 2H), 1.08 (d, J=8.2 Hz, 2H), 0.94 (s, 9H), 0.82 (d, J=4.2 Hz, 2H).

### Example A177. N6-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-No-methylquinoline-3,6-dicarboxamide

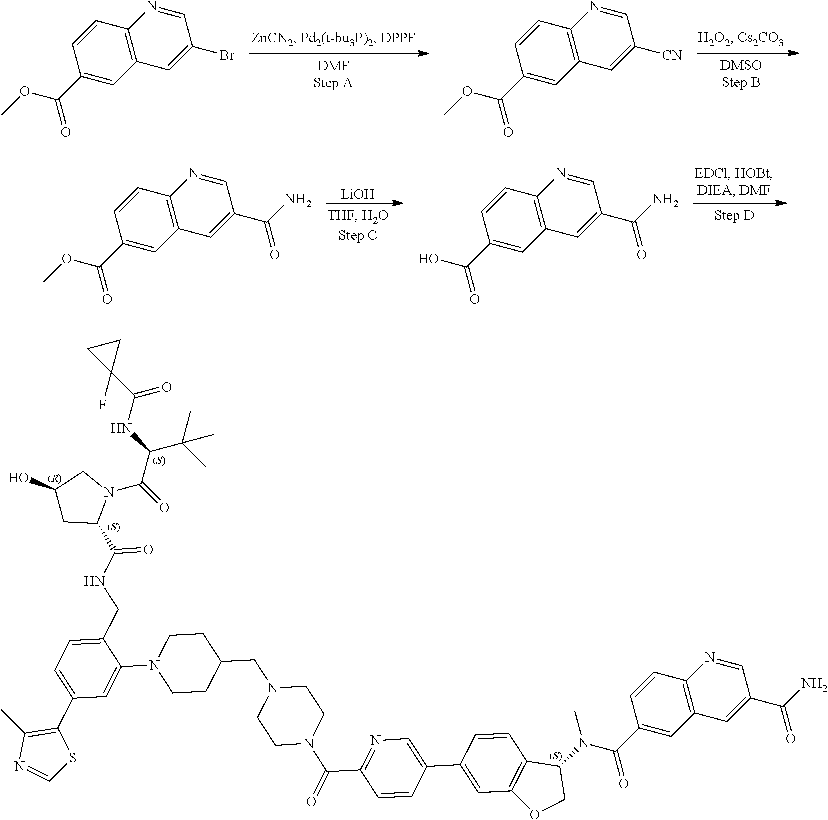

Step A: methyl 3-cyanoquinoline-6-carboxylate

To a solution of methyl 3-bromoquinoline-6-carboxylate (1 g, 3.76 mmol, 1.0 eq.) in DMA (10 mL) was added DPPF (417 mg, 752 μmol, 0.2 eq.), Zn(CN)₂ (883 mg, 7.52 mmol, 2.0 eq.) and Pd₂(t-busP)₂ (192 mg, 376 μmol, 0.1 eq.) under N₂. The mixture was stirred at 110° C. for 2 hours to give yellow solution. The reaction mixture was quenched with water (50 mL), and then extracted with EtOAc (50 mL×3). The combined organic layers were washed with brine three times, dried over anhydrous Na₂SO₄ and concentrated. The residue was purified by column chromatography (silica gel, 0% EtOAc in PE~100% EtOAc in PE) to afford methyl 3-cyanoquinoline-6-carboxylate (600 mg, 71% yield) as a yellow solid. LC-MS (ESI): mass calcd. For C₁₂H₈N₂O₂, 212.1; m/z found, 213.1 [M+H]⁺.

Step B: methyl 3-carbamoylquinoline-6-carboxylate

To a mixture of methyl 3-cyanoquinoline-6-carboxylate (300 mg, 1.41 mmol, 1.00 eq.) in DMSO (10 mL) was added Cs₂CO₃ (921 mg, 2.83 mmol, 2.0 eq.) and H₂O₂ (173 μL, 30% Wt in water, 5.65 mmol, 4.0 eq.) under N₂. The mixture was stirred at 40° C. for 2 hours to give yellow solution. The reaction mixture was quenched with water (50 mL), and then extracted with EtOAc (50 mL×3). The combined organic layers were washed with brine three times, dried over anhydrous Na₂SO₄ and concentrated. The residue was purified by column chromatography (silica gel, 0% EtOAc in PE~100% EtOAc in PE) to afford methyl 3-carbamoylquinoline-6-carboxylate (80.0 mg, 23% yield) as a yellow solid. LC-MS (ESI): mass calcd. For C₁₂H₁₀N₂O₃, 230.1; m/z found, 231.1 [M+H]⁺.

Steps C-D: N6-(S)-6-16-(4-(1-(2-(((28, 4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N6-methylquinoline-3,6-dicarboxamide

Example A177 was synthesized following the same procedures as described in Example A51 (2.1 mg, 3% yield over 2 steps) as a white solid. LC-MS (ESI): mass calcd. for C₆₂H₇FN₁₁O₈S, 1147.5; m/z found, 1148.5 [M+H]⁺. ¹H NMR (400 MHz, DMSO-d₆) δ 9.72 (s, 1H), 9.37 (d, J=1.9 Hz, 1H), 8.99 (s, 1H), 8.94 (s, 1H), 8.52 (s, 1H), 8.36 (s, 1H), 8.29 (d,)=10.9 Hz, 2H), 8.18 (s, 1H), 7.98 (d, J=8.4 Hz, 1H), 7.81-7.67 (m, 2H), 7.62 (d, J=7.5 Hz, 1H), 7.44 (d, J=8.3 Hz, 2H), 7.36 (s, 1H), 7.27 (dd, J=9.2, 2.4 Hz, 1H), 7.14 (d, J=6.1 Hz, 2H), 4.74 (dd, J=10.3, 4.0 Hz, 1H), 4.60 (d, J=9.5 Hz, 2H), 4.49 (t, J=8.0 Hz, 2H), 4.40-4.33 (m, 2H), 3.70-3.54 (m, 6H), 3.57-3.47 (m, 1H), 3.44-3.28 (m, 2H), 3.27-3.03 (m, 6H), 2.82-2.61 (m, 5H), 2.46 (s, 3H), 2.12-2.03 (m, 1H), 2.03-1.96 (m, 2H), 1.94-1.82 (m, 2H), 1.51-1.30 (m, 4H), 1.26-1.19 (m, 2H), 0.97 (s, 9H).

### Example A178. 2-amino-3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-isobutylquinoline-6-carboxamide

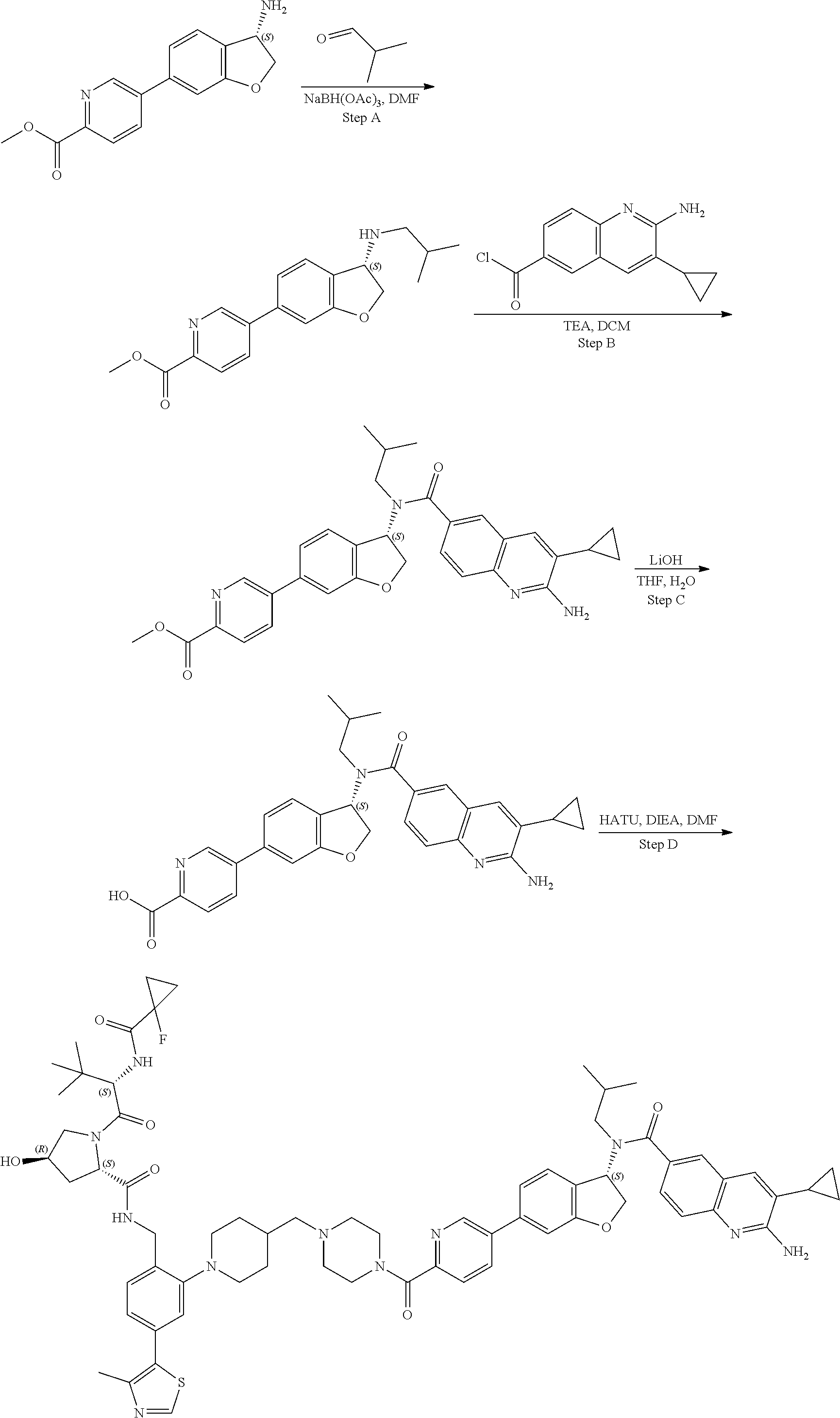

Example A178 was synthesized following the same procedures as described in Example A45 (1.3 mg, 1% yield over 4 steps) as a white solid, LC-MS (ESI): mass calcd. for C₆₇H₈₀F₁₁O₇S, 1201.59; m/z found, 1203.0 [M+H]⁺. ¹HNMR (400 MHz, DMSO-d₆) δ 9.49 (s, 1H), 8.99 (s, 1H), 8.93 (s, 1H), 8.74 (s, 1H), 8.51 (t, J=6.0 Hz, 1H), 8.28 (d, J=9.3 Hz, 1H), 8.22 (s, 1H), 7.99 (s, 1H), 7.77 (d, J=8.1 Hz, 3H), 7.59 (d, J=7.8 Hz, 1H), 7.44 (d, J=8.3 Hz, 1H), 7.38 (d, J=8.3 Hz, 1H), 7.32 (s, 1H), 7.28-7.25 (m, 1H), 7.15-7.11 (m, 2H), 4.70-4.57 (m, 4H), 4.49 (t, J=7.9 Hz, 1H), 4.38-4.34 (m, 3H), 4.29-4.18 (m, 1H), 3.69-3.59 (m, 6H), 3.22-3.04 (m, 7H), 2.73-2.66 (m, 4H), 2.46 (s, 3H), 2.11-2.07 (m, 1H), 1.94-1.86 (m, 4H), 1.51-1.31 (m, 6H), 1.26-1.19 (m, 4H), 1.09-1.06 (m, 2H), 0.97 (s, 9H), 0.81 (d, J=4.1 Hz, 3H), 0.70-0.64 (m, 3H).

### Example A179. 2-amino-3-cyclopropyl-N-((S)-6-(6-(4-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)but-3-yn-1-yl)piperazine-1-carbonyl)pyridin-3-yl)-2,3-dihydrobenzofuran-3-yl)-N-methylquinoline-6-carboxamide

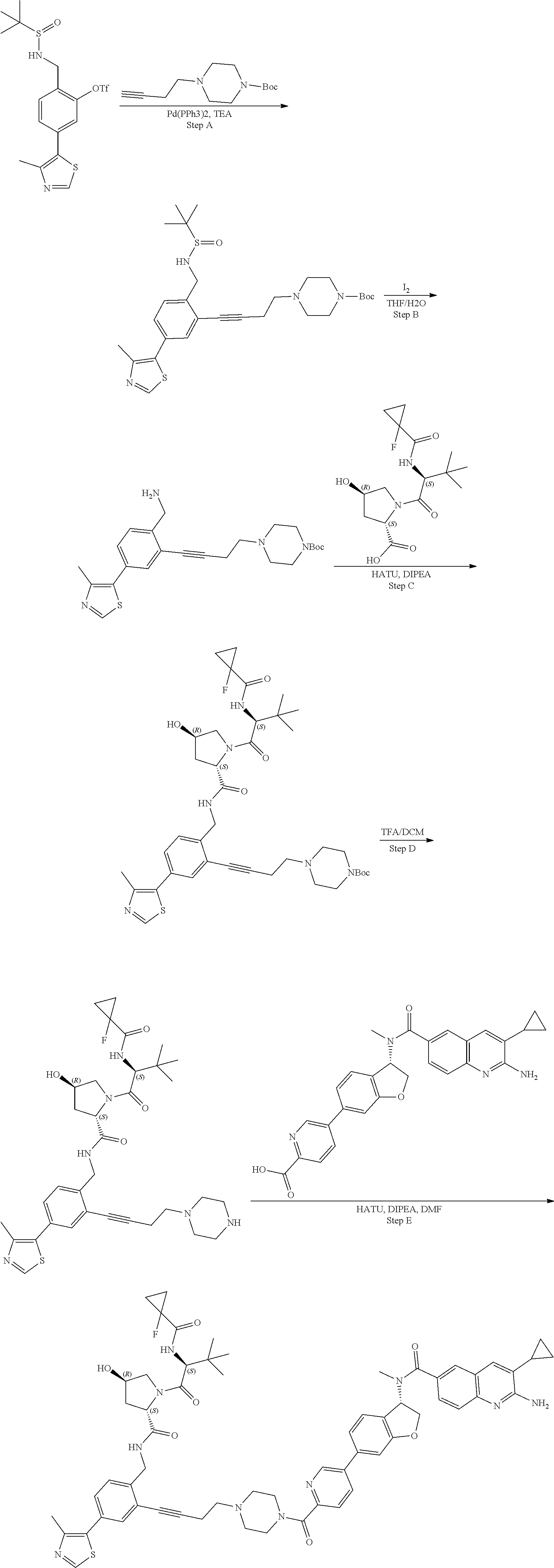

Example A179 was synthesized following the same procedures as described in Example A150 (4.5 mg, 8% yield over 5 steps) as a white solid. LC-MS (ESI): mass calcd. For C₆₂H₆₇FN₁₀O₇S, 1114.49; m/z found, 1115.49 [M+H]⁺. ¹H NMR (400 MHz, DMSO-d₆) δ 9.01 (s, 1H), 8.92 (s, 1H), 8.84 (s, 2H), 8.70 (t, J=5.9 Hz, 1H), 8.27 (d, J=8.1 Hz, 1H), 8.21 (s, 1H), 8.04 (s, 1H), 7.86 (d, J=8.5 Hz, 1H), 7.77 (d, J=8.1 Hz, 2H), 7.59-7.52 (m, 2H), 7.48 (s, 1H), 7.41 (t, J=9.2 Hz, 2H), 7.35 (s, 1H), 7.29 (d, J=6.7 Hz, 1H), 4.82 (s, 1H), 4.72-4.55 (m, 3H), 4.52 (t, J=8.1 Hz, 1H), 4.47-4.33 (m, 3H), 3.68-3.58 (m, 4H), 3.34-3.12 (m, 9H), 3.02 (t, J=8.2 Hz, 2H), 2.66 (s, 4H), 2.44 (s, 3H), 2.15-2.05 (m, 1H), 1.94-1.87 (m, 2H), 1.42-1.32 (m, 2H), 1.24-1.20 (m, 2H), 1.08 (d, J=8.1 Hz, 2H), 0.94 (s, 9H), 0.81 (d, J=3.5 Hz, 2H).

### Example A180. 2-amino-3-cyclopropyl-N-((S)-6-(6-(4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-3,6-dihydropyridin-1 (2H)-yl)methyl)piperidine-1-carbonyl)pyridin-3-yl)-2,3-dihydrobenzofuran-3-yl)-N-methylquinoline-6-carboxamide

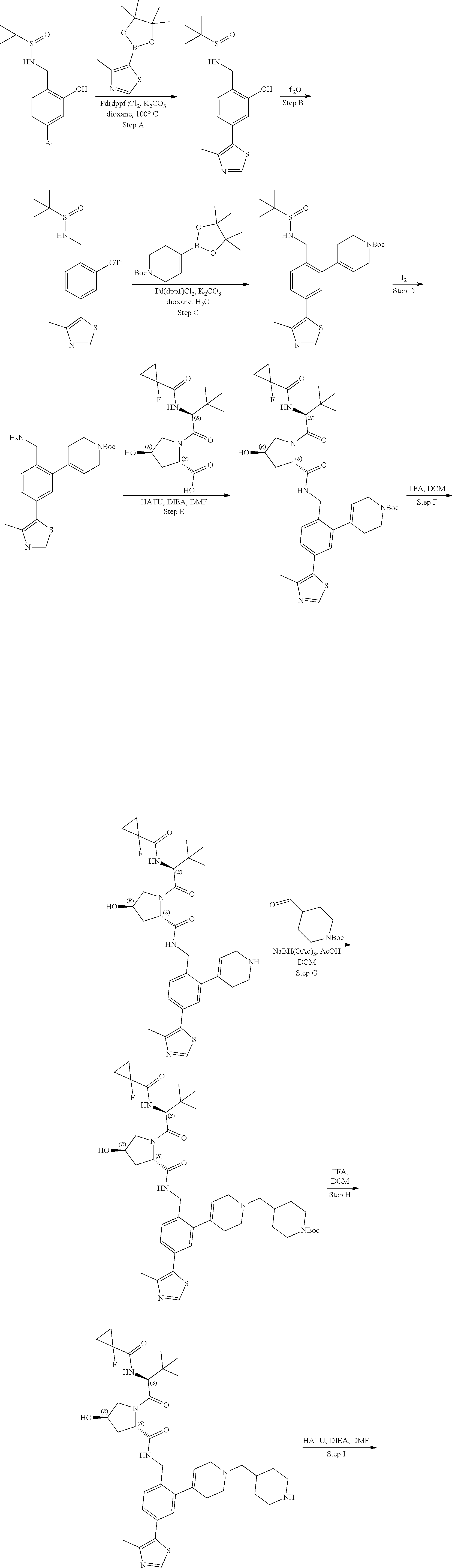

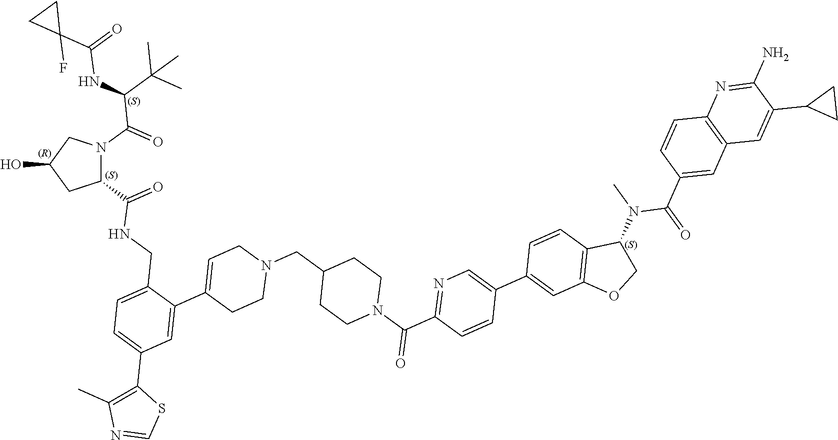

Example A180 was synthesized following the same procedures as described in Example A145 (29.2 mg, 3% yield over 9 steps) as a white solid. LC-MS (ESI): mass calcd. for C₆₅H₇₃FN₁₀O₇S, 1156.54; m/z found, 579.6 [1/2 M+H]⁺. ¹H NMR (400 MHz, DMSO-d₆) δ 9.57 (s, 1H), 9.01 (s, 1H), 8.90 (s, 2H), 8.56 (d, J=5.8 Hz, 1H), 8.27-8.17 (m, 2H), 8.05 (s, 1H), 7.90-7.83 (m, 1H), 7.76 (s, 1H), 7.65 (d, J=8.2 Hz, 1H), 7.58-7.48 (m, 2H), 7.43-7.30 (m, 3H), 7.25 (s, 2H), 5.70 (s, 1H), 4.88-4.74 (m, 2H), 4.72-4.65 (m, 2H), 4.61-4.54 (m, 2H), 4.51-4.43 (m, 2H), 4.39-4.31 (m, 2H), 4.29-4.21 (m, 1H), 4.09-4.02 (m, 1H), 3.86-3.75 (m, 2H), 3.70-3.59 (m, 3H), 3.40-3.30 (m, 1H), 3.22-3.08 (m, 3H), 2.88 (t, J=12.0 Hz, 1H), 2.69-2.62 (m, 3H), 2.46 (s, 3H), 2.29-2.18 (m, 1H), 2.13-2.03 (m, 1H), 2.01-1.85 (m, 3H), 1.84-1.71 (m, 1H), 1.41-1.25 (m, 4H), 1.24-1.20 (m, 2H), 1.08 (d, J=8.0 Hz, 2H), 0.97 (s, 9H), 0.82 (d, J=4.3 Hz, 2H).

### Example A181. 3-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

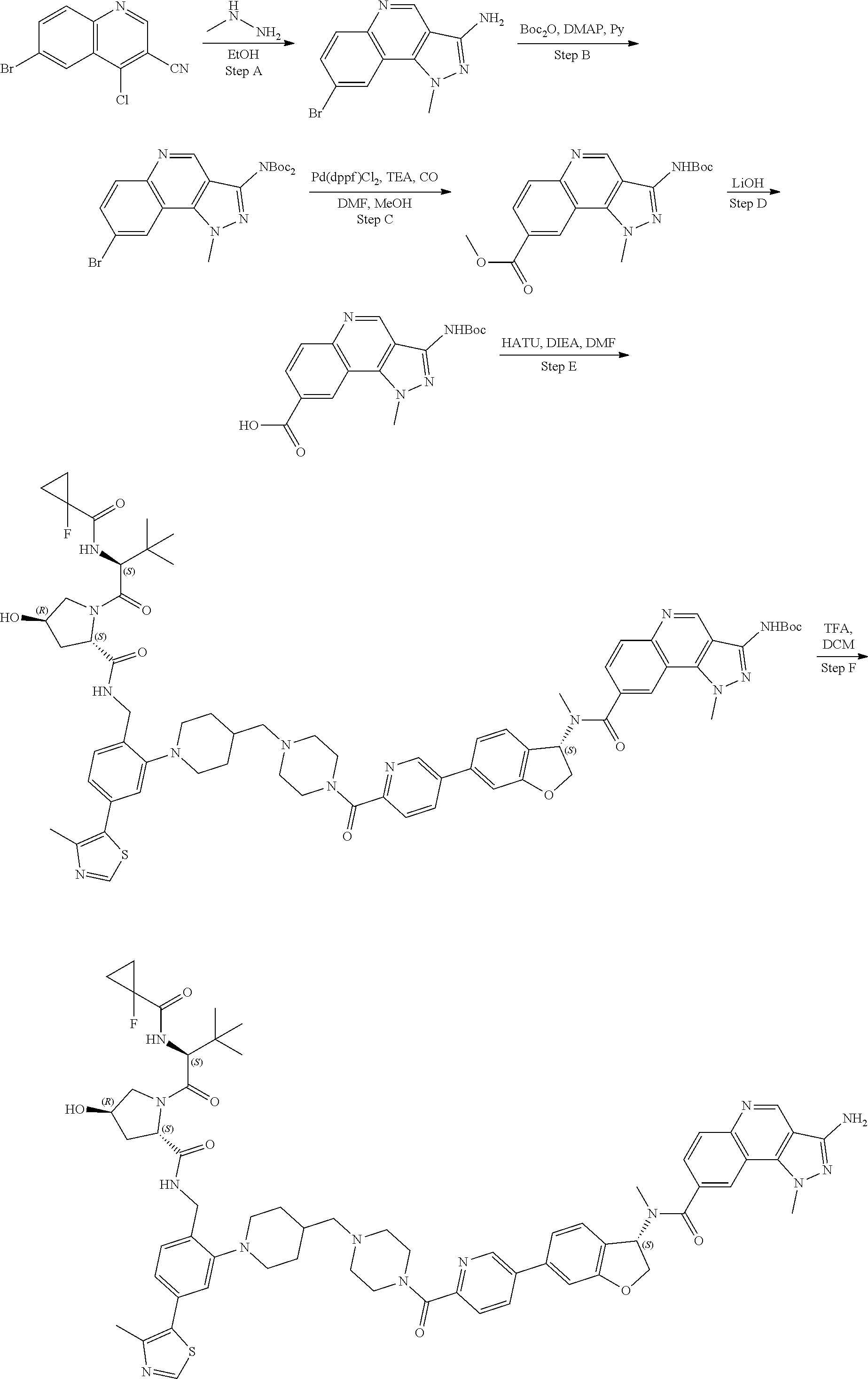

Step A: 8-bromo-1-methyl-1H-pyrazolo[4,3-c]quinolin-3-amine

To a solution of 6-bromo-4-chloroquinoline-3-carbonitrile (1 g, 3.74 mmol, 1.0 eq.) in EtOH (10 mL) was added methylhydrazine (861 mg, 7.48 mmol, 2.0 eq.) at room temperature. The mixture was then stirred at 80° C. for 16 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% MeOH in DCM~8% MeOH in DCM) to afford 8-bromo-1-methyl-1H-pyrazolo[4,3-c]quinolin-3-amine (600 mg, 58% yield) as a yellow solid. LC-MS (ESI): mass calcd. For CH₉BrN₄, 276.0/278.0; m/z found, 276.8/278.8 [M+H]⁺.

Step B: tert-butyl (8-bromo-1-methyl-1H-pyrazolo[4,3-c]quinolin-3-yl)(tert-butoxycarbonyl)carbamate

To a solution of 8-bromo-1-methyl-1H-pyrazolo[4,3-c]quinolin-3-amine (100 mg, 361 μmol, 1.0 eq.) in DCM (5 mL) was added DMAP (9 mg, 72 μmol, 0.2 eq.), Py (71 mg, 902 μmol, 2.5 eq.) and Di-tert-butyl dicarbonate (118 mg, 541 μmol, 1.5 eq.) at room temperature. The mixture was then stirred at room temperature for 2 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~15% EtOAc in PE) to afford tert-butyl (8-bromo-1-methyl-1H-pyrazolo[4,3-c]quinolin-3-yl)(tert-butoxycarbonyl)carbamate (80 mg, 46% yield) as a white solid. LC-MS (ESI): mass calcd. For C₂₁H₂₅BrN₄O₄, 476.1/478.1; m/z found, 477.1/479.1 [M+H]⁺.

Step C: methyl 3-((tert-butoxycarbonyl)amino)-1-methyl-1H-pyrazolo[4,3-c]quinoline-8-carboxylate

To a solution of tert-butyl (8-bromo-1-methyl-1H-pyrazolo[4,3-c]quinolin-3-yl)(tert-butoxycarbonyl)carbamate (250 mg, 524 μmol, 1.0 eq.) in DMF (5 mL) and MeOH (5 mL) was added 1,1′-Bis(diphenylphosphino) ferrocene-palladium (II) dichloride (38 mg, 52.4 μmol, 0.1 eq.) and TEA (159 mg, 1.57 mmol, 3.0 eq.) at room temperature. The mixture was then stirred at 100° C. for 16 hours under CO atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~40% EtOAc in PE) to afford methyl 3-((tert-butoxycarbonyl)amino)-1-methyl-1H-pyrazolo[4,3-c]quinoline-8-carboxylate (100 mg, 53% yield) as a white solid. LC-MS (ESI): mass calcd. For C₁₈H₂₀N₄O₄, 356.1; m/z found, 357.1 [M+H]⁺.

Steps D-F: 3-amino-N-(S)-6-16-(4-((1-(2-(2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

Example A181 was synthesized following the same procedures as described in Example A107 (10.5 mg, 14% yield over 3 steps) as a yellow solid. LC-MS (ESI): mass calcd. for C₆₃H₇₂FN₁₃O₇S, 1173.5/z found, 1174.1 [M+H]⁺. ¹H NMR (400 MHz, DMSO-d₆) δ 10.46 (s, 1H), 9.58 (s, 1H), 9.00 (s, 1H), 8.94 (s, 1H), 8.72-8.51 (m, 2H), 8.28 (s, 2H), 8.14 (s, 1H), 7.78 (d, J=7.6 Hz, 1H), 7.65 (s, 1H), 7.47-7.32 (m, 3H), 7.30-7.24 (m, 1H), 7.15 (d, J=7.9 Hz, 2H), 6.47 (s, 1H), 5.64 (s, 1H), 4.89-4.85 (m, 1H), 4.80-4.78 (m, 1H), 4.71-4.65 (m, 1H), 4.62-4.59 (m, 1H), 4.52-4.47 (m, 1H), 4.42-4.32 (m, 6H), 4.24-4.17 (m, 1H), 3.78-3.58 (m, 4H), 3.56-3.42 (m, 2H), 3.25-3.05 (m, 6H), 2.82-2.71 (m, 3H), 2.71-2.60 (m, 2H), 2.47 (s, 3H), 2.12-1.89 (m, 5H), 1.54-1.41 (m, 2H), 1.41-1.30 (m, 2H), 1.25-1.19 (m, 2H), 0.98 (s, 9H).

### Example A182. 4-amino-3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-3H-pyrrolo[2,3-c]quinoline-8-carboxamide

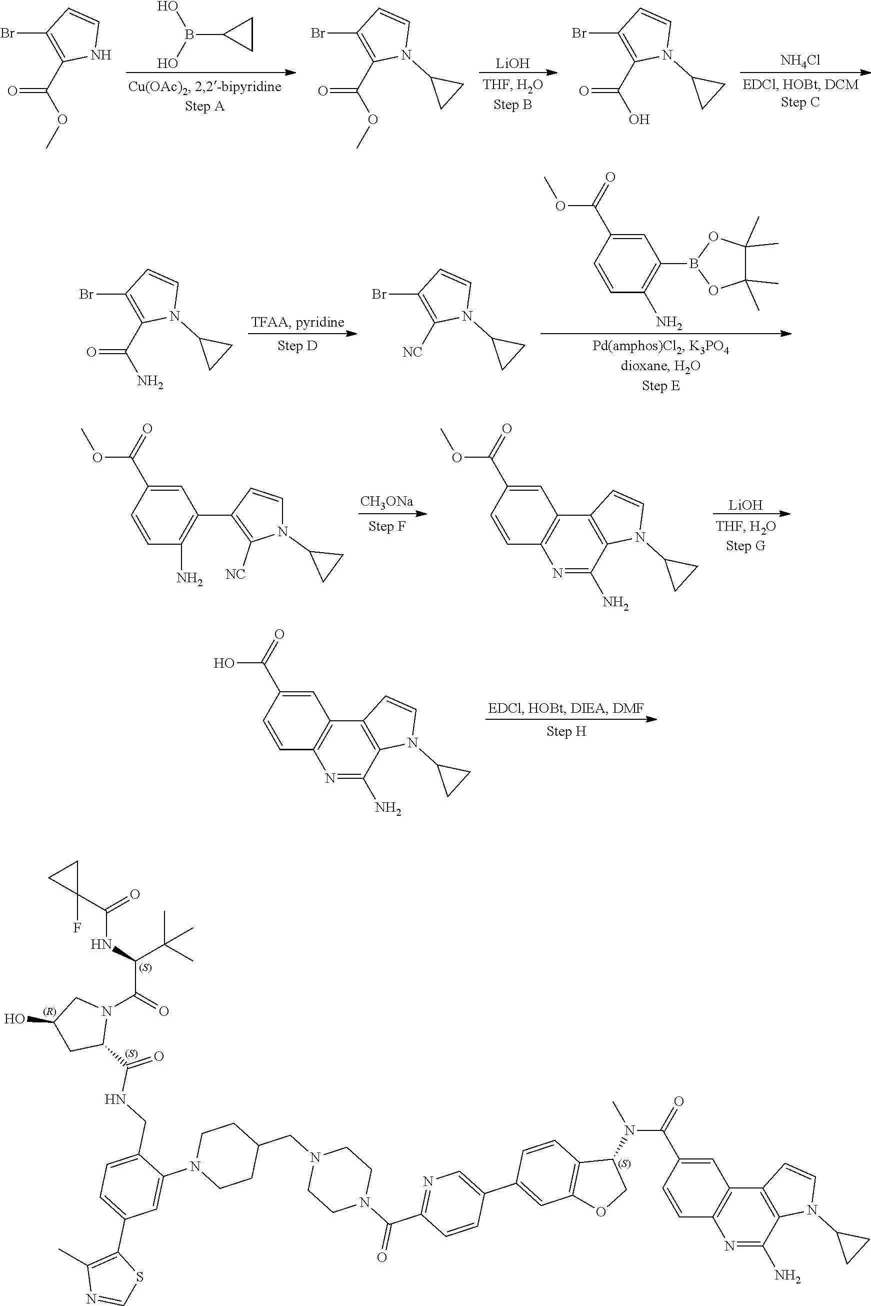

Step A: methyl 3-bromo-1-cyclopropyl-1H-pyrrole-2-carboxylate

To a solution of methyl 3-bromo-1H-pyrrole-2-carboxylate (2.00 g, 9.80 mmol, 1.0 eq.), cyclopropylboronic acid (1.68 g, 19.6 mmol, 2.0 eq.), Copper diacetate (1.78 g, 0.40 mL, 9.80 mmol, 1.0 eq.), 2,2′-bipyridine (1.53 g, 9.80 mmol, 1.0 eq.) and Na₂CO₃ (2.08 g, 19.6 mmol, 2.0 eq.) in DCE (40.0 mL) was stirred at 70° C. for 16 hours under 02. The reaction was filtered and the filtrate was concentrated in vacuo to give a residue, the residue was purified by silica gel chromatography (100-200 mesh silica gel, 0-50% EtOAc in PE) to give methyl 3-bromo-1-cyclopropyl-1H-pyrrole-2-carboxylate (1.50 g, 63% yield) as a yellow oil. LC-MS (ESI): mass calcd. For C₉H₁₀BrNO₂, 242.99; m/z found, 244.3 [M+H]⁺.

Step B: 3-bromo-1-cyclopropyl-1H-pyrrole-2-carboxylic acid

To a solution of methyl 3-bromo-1-cyclopropyl-1H-pyrrole-2-carboxylate (1.30 g, 5.33 mmol, 1.0 eq.) and LiOH (383 mg, 16.0 mmol, 3.0 eq.) in THF (10 mL), H₂O (2 mL) and MeOH (4 mL) was stirred at 80° C. for 16 hours. The reaction was diluted with water (10 mL), extracted with EtOAc (30 mL×3), the aqueous phase was adjusted pH to 3-4 by IN HCl (aq.), then it was extracted with EtOAc (30 mL), the organic phase was dried over Na₂SO₄, filtered and concentrated in vacuo, the residue was purified by silica gel chromatography (100-200 mesh silica gel, 0-40% EtOAc in PE) to give 3-bromo-1-cyclopropyl-1H-pyrrole-2-carboxylic acid (1.10 g, 90% yield) as a yellow solid. LC-MS (ESI): mass calcd. For C₈H₈BrNO₂, 228.97; m/z found, 230.1 [M+H]⁺.

Step C: 3-bromo-1-cyclopropyl-1H-pyrrole-2-carboxamide

To a solution of 3-bromo-1-cyclopropyl-1H-pyrrole-2-carboxylic acid (1.10 g, 4.78 mmol, 1.0 eq.), 1-(3-Dimethylaminopropyl)-3-ethylcarbodiimideHydrochloride (EDCI)(1.37 g, 7.17 mmol, 1.5 eq.), 1-Hydroxy-1H-benzotriazole (969 mg, 7.17 mmol, 1.5 eq.) and Ammonium chloride (512 mg, 9.56 mmol, 2.0 eq.) in DMF (15 mL) was added N-ethyl-N-isopropylpropan-2-amine (1.85 g, 14.3 mmol, 3.0 eq.), the reaction was stirred at room temperature for 2 hours. The reaction was quenched with water (20 mL), extracted with EtOAc (30 mL×3), the organic phase was dried over Na₂SO₄, filtered and concentrated in vacuo, the residue was purified by silica gel chromatography (100-200 mesh silica gel, 0-30% EtOAc in PE) to give 3-bromo-1-cyclopropyl-1H-pyrrole-2-carboxamide (1.00 g, 91% yield) as a yellow solid. LC-MS (ESI): mass calcd. For C₈H₉BrN₂O, 227.99; m/z found, 229.1 [M+H]⁺.

Step D: 3-bromo-1-cyclopropyl-1H-pyrrole-2-carbonitrile

To a solution of 3-bromo-1-cyclopropyl-1H-pyrrole-2-carboxamide (0.90 g, 3.9 mmol, 1.0 eq.) in THF (14 mL) was added pyridine (0.78 g, 9.8 mmol, 2.5 eq.) and 2,2,2-trifluoroacetic anhydride (2.1 g, 9.8 mmol, 2.5 eq.), the reaction was concentrated in vacuo, the residue was purified by silica gel chromatography (100-200 mesh silica gel, 0-30% EtOAc in PE) to give 3-bromo-1-cyclopropyl-1H-pyrrole-2-carbonitrile (630 mg, 76% yield) as a yellow oil. LC-MS (ESI): mass calcd. For C₈H₇BrN₂, 209.89; m/z found, 211.1 [M+H]⁺.

Step E-H: 4-amino-3-cyclopropyl-N-(S)-6-(6-(4-(1-(2-(((28,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-3H-pyrrolo[2,3-c]quinoline-8-carboxamide

Example A182 was synthesized following the same procedures as described in Example A23 (9.5 mg, 2.6% yield over 4 steps) as a white solid. LC-MS (ESI): mass calcd. for C₆₆H₇₅FN₁₂O₇S, 1198.56 m/z found, 1199.2 [M+H]⁺. ¹H NMR (400 MHz, DMSO-d₆) δ 9.00 (s, 1H), 8.94 (s, 1H), 8.54 (s, 3H), 8.40 (s, 1H), 8.28 (d, J=7.6 Hz, 1H), 7.92 (d, J=2.8 Hz, 1H), 7.80-7.73 (m, 3H), 7.60 (d, J=7.7 Hz, 1H), 7.45 (d, J=8.1 Hz, 1H), 7.43-7.39 (m, 1H), 7.36 (s, 1H), 7.31 (d, J=2.8 Hz, 1H), 7.29-7.25 (m, 1H), 7.17-7.13 (m, 2H), 4.88-4.82 (m, 1H), 4.73-4.71 (m, 1H), 4.60 (s, 1H), 4.52-4.47 (m, 1H), 4.38-4.34 (m, 4H), 4.25-4.21 (m, 2H), 4.04-4.00 (m, 2H), 3.69-3.60 (m, 4H), 3.55-3.49 (m, 1H), 3.44-3.37 (m, 1H), 3.18-3.12 (m, 4H), 2.77-2.71 (m, 2H), 2.68 (s, 3H), 2.47 (s, 3H), 2.12-2.07 (m, 1H), 2.03-1.98 (m, 1H), 1.95-1.88 (m, 3H), 1.55-1.44 (m, 2H), 1.40-1.33 (m, 2H), 1.31-1.20 (m, 7H), 0.97 (s, 9H).

### Example A183. 4-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylimidazo[1,2-a]quinoxaline-8-carboxamide

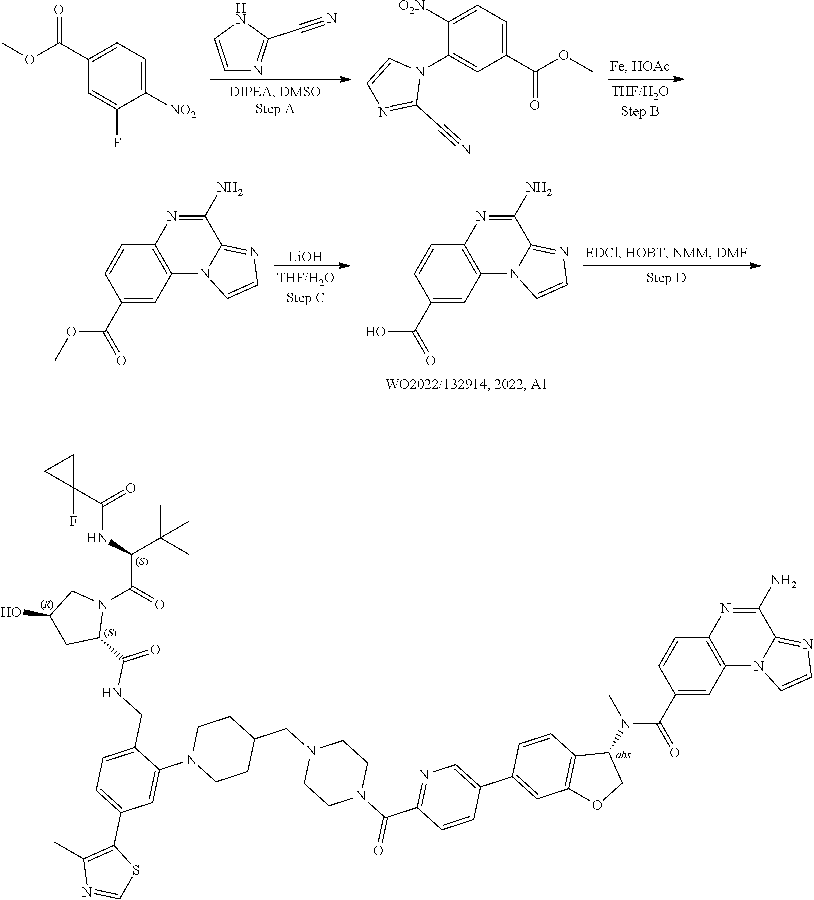

Example A183 was synthesized following a similar procedure as described in WO2022/132914 (1.1 mg, 1% yield over 4 steps) as a white solid. LC-MS (ESI): mass calcd. For C₆₂H₇₀FN₁₃O₇S, 1159.52; m/z found, 1160.52 [M+H]⁺. ¹H NMR (400 MHz, DMSO-d₆) δ 9.55 (s, 1H), 8.99 (s, 1H), 8.94 (s, 1H), 8.87 (s, 1H), 8.65 (s, 1H), 8.51 (t, J=7.6 Hz, 1H), 8.41 (s, 1H), 8.29 (d, J=6.2 Hz, 1H), 7.83-7.74 (m, 2H), 7.71-7.63 (m, 2H), 7.59 (d, J=7.7 Hz, 1H), 7.45-7.40 (m, 2H), 7.36 (s, 1H), 7.27 (d, J=7.0 Hz, 1H), 7.13 (d, J=4.4 Hz, 2H), 4.71 (d, J=8.4 Hz, 1H), 4.60 (d, J=9.1 Hz, 2H), 4.49 (t, J=8.2 Hz, 1H), 4.35 (d, J=5.7 Hz, 2H), 4.24 (d, J=13.3 Hz, 1H), 3.64 (d, J=13.5 Hz, 6H), 3.22-3.05 (m, 9H), 2.76-2.63 (m, 6H), 2.46 (s, 3H), 2.11-2.06 (m, 1H), 2.01-1.96 (m, 1H), 1.93-1.83 (m, 3H), 1.51-1.42 (m, 2H), 1.41-1.33 (m, 2H), 1.22 (dd, J=8.3 Hz, 2H), 0.97 (s, 9H).

### Example A184. N1-(8-aminoimidazo[1,5-a]pyrazin-5-yl)-N2-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N2-methyloxalamide

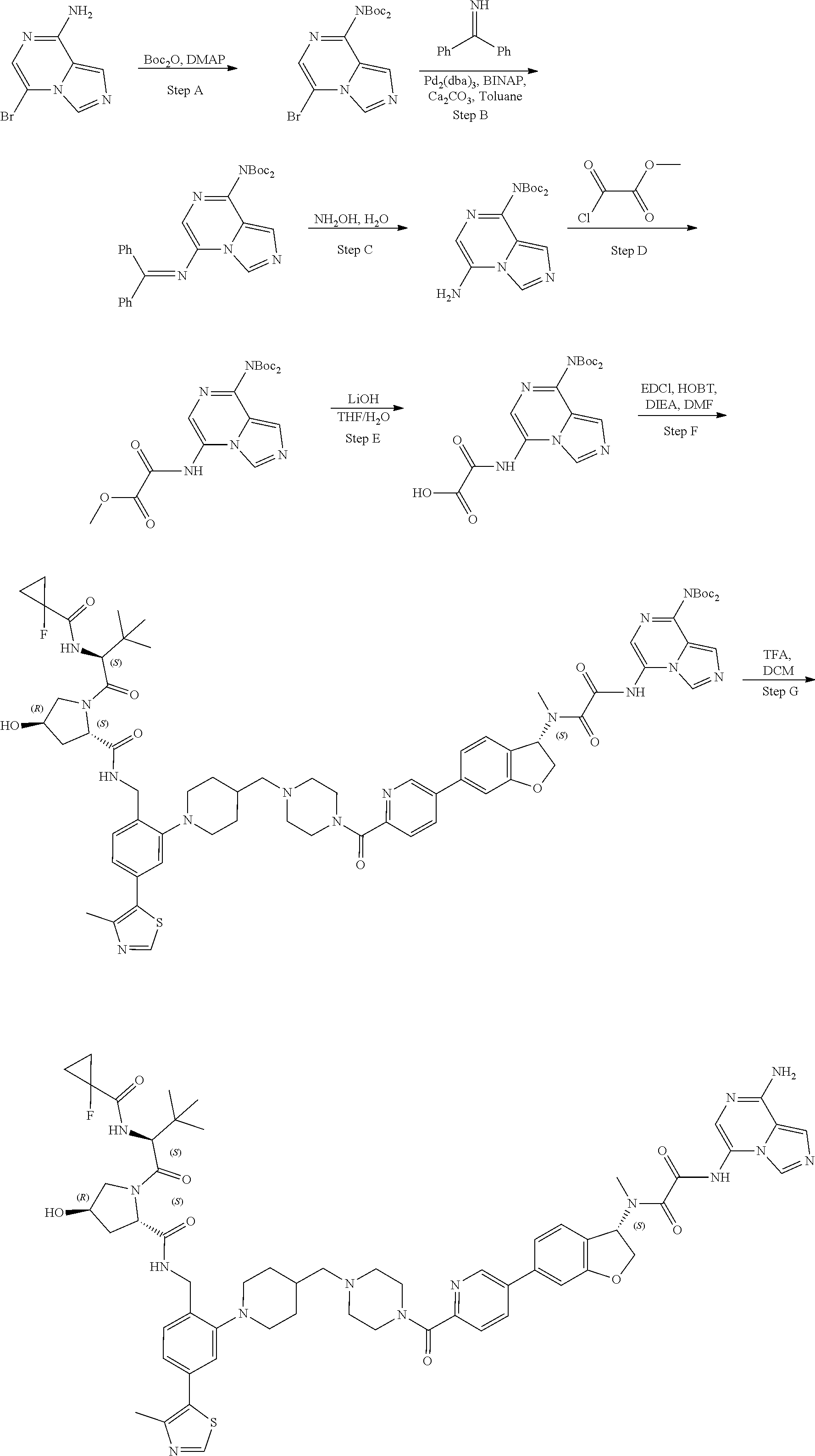

Step A: tert-butyl (5-bromoimidazo[1,5-a]pyrazin-8-yl)(tert-butoxycarbonyl)carbamate

To a solution of 5-bromoimidazo[1,5-a]pyrazin-8-amine (300 mg, 1.41 mmol, 1.0 eq.) in DCM (10 mL) was added TEA (589 μL, 4.22 mmol, 3.0 eq.), DMAP (43 mg, 352 μmol, 0.25 eq.) and Boc₂O (809 μL, 3.52 mmol, 2.5 eq.) under N₂. The mixture was stirred at room temperature for 16 hours. The reaction mixture was quenched with water (50 mL), and then extracted with DCM (50 mL×2). The combined organic layers were washed with brine three times, dried over anhydrous Na₂SO₄ and concentrated. The residue was purified by column chromatography (silica gel, 0% EtOAc in PE~100% EtOAc in PE) to afford tert-butyl (5-bromoimidazo[1,5-a]pyrazin-8-yl)(tert-butoxycarbonyl)carbamate (400 mg, 65% yield) as a yellow solid. LC-MS (ESI): mass calcd. For C₁₆H₂₁BrN₄O₄, 413.3; m/z found, 413.3/415.3 [M+H]⁺.

Step B: tert-butyl (tert-butoxycarbonyl)(5-((diphenylmethylene)amino) imidazo[1,5-a]pyrazin-8-yl)carbamate

To solution of tert-butyl (5-bromoimidazo[1,5-a]pyrazin-8-yl)(tert-butoxycarbonyl)carbamate (400 mg, 968 μmol, 1.0 eq.) in Toluene (10 mL) was added Pd₂(dba)₃ (88.6 mg, 96.8 μmol, 0.1 eq.), diphenylmethanimine (351 mg, 1.94 mmol, 2.0 eq.), BINAP (121 mg, 194 μmol, 0.2 eq.) and Cs₂CO₃ (946 mg, 2.90 mmol, 3.0 eq.) under N₂. The mixture was stirred at 100° C. for 16 hours to give yellow solution. The reaction mixture was quenched with water (50 mL), and then extracted with EtOAc (50 mL×3). The combined organic layers were washed with brine three times, dried over anhydrous Na₂SO₄ and concentrated. The residue was purified by column chromatography (silica gel, 0% EtOAc in PE~100% EtOAc in PE) to afford tert-butyl (tert-butoxycarbonyl)(5-((diphenylmethylene)amino) imidazo[1,5-a]pyrazin-8-yl)carbamate (200 mg, 28% yield) as a yellow solid. LC-MS (ESI): mass calcd. For C₂₉H₃₁N₅O₄, 513.2; m/z found, 514.2 [M+H]⁺

Step C: methyl 3-(difluoromethyl)-2-((4-methoxybenzyl)amino) quinoline-6-carboxylate

A solution of tert-butyl (tert-butoxycarbonyl)(5-((diphenylmethylene)amino) imidazo[1,5-a]pyrazin-8-yl)carbamate (150 mg, 292 μmol, 1.0 eq.) in 2N HCl (2 mL) and MeOH (4 mL) was stirred at 0° C. for 2 hours to give yellow solution. The mixture was quenched NaHCO₃ solution adjust pH=8-9, and then extracted with EtOAc (50 mL×3). The combined organic layers were washed with brine three times, dried over anhydrous Na₂SO₄ and concentrated. The residue was purified by column chromatography (silica gel, 0% EtOAc in PE~100% EtOAc in PE) to afford tert-butyl (5-aminoimidazo[1,5-a]pyrazin-8-yl)(tert-butoxycarbonyl)carbamate (80 mg, 74% yield) as a yellow solid. LC-MS (ESI): mass calcd. For C₁₆H₂₃N504, 349.2; m/z found, 350.2 [M+H]⁺.

Step D-F: N1-(8-aminoimidazo[1,5-a]pyrazin-5-yl)-N2-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N2-methyloxalamide

Example A184 was synthesized following the same procedures as described in Example A44 (2.7 mg, 1% yield over 4 steps) as a yellow solid. LC-MS (ESI): mass calcd. for C₅₉H₆₉FN₁₄O₈S, 1152.5; m/z found, 1153.5 [M+H]⁺. ¹H NMR (400 MHz, DMSO-d₆) δ 11.47 (d, J=16.3 Hz, 1H), 10.03 (s, 1H), 8.99 (s, 1H), 8.95 (s, 1H), 8.56 and 8.56 (s x2, 1H), 8.53 (s, 1H), 8.44 (d, J=5.6 Hz, 1H), 8.32-8.26 (m, 1H), 7.82-7.76 (m, 1H), 7.60 and 7.49 (d x2, J=7.9 Hz, 1H), 7.47-7.36 (m, 4H), 7.32 (d, J=3.2 Hz, 1H), 7.30-7.24 (m, 2H), 7.14 (d, J=6.4 Hz, 2H), 4.85-4.78 (m, 1H), 4.74-4.66 (m, 1H), 4.60 (d, J=9.1 Hz, 3H), 4.53-4.46 (m, 2H), 4.38-4.32 (m, 3H), 4.27-4.15 (m, 1H), 3.73-3.34 (m, 8H), 3.21-3.06 (m, 6H), 2.85-2.63 (m, 5H), 2.46 (s, 3H), 2.14-2.05 (m, 1H), 2.03-1.96 (m, 1H), 1.96-1.87 (m, 3H), 1.53-1.42 (m, 2H), 1.41-1.32 (m, 2H), 1.22 (dd, J=8.1, 2.6 Hz, 2H), 0.98 (s, 9H).

### Example A185. 2-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-3-(prop-1-yn-1-yl)quinoline-6-carboxamide

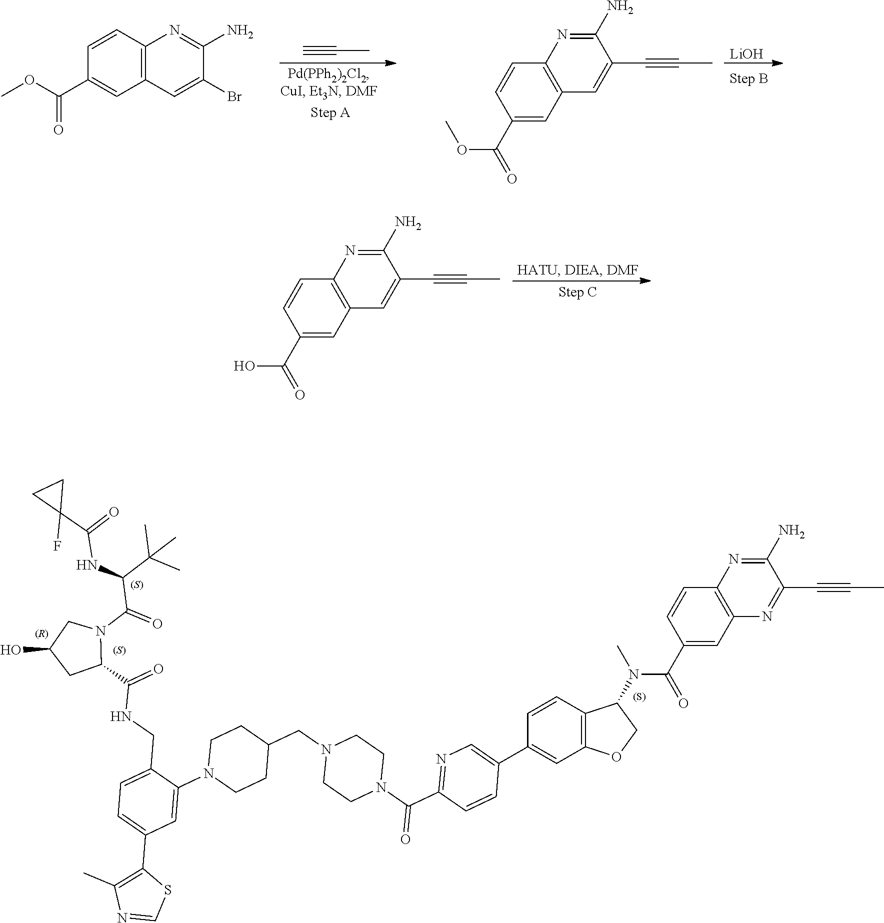

Example A185 was synthesized following the same procedures as described in Example A71 (3.0 mg, 3% yield over 3 steps) as a white solid. LC-MS (ESI): mass calcd. for C₆₄H₇₂FN₁₁O₇S, 1157.53; m/z found, 1159.0 [M+H]⁺. ¹H NMR (400 MHz, DMSO-d₆) δ 9.55 (s, 1H), 8.99 (s, 1H), 8.93 (d, J=1.4 Hz, 1H), 8.61-8.47 (m, 3H), 8.28 (dd, J=8.2, 2.1 Hz, 1H), 8.04 (s, 1H), 7.87 (dd, J=8.2, 0.8 Hz, 1H), 7.78 (d, J=8.2 Hz, 1H), 7.75-7.69 (m, 1H), 7.56 (d, J=7.7 Hz, 1H), 7.44 (d, J=8.3 Hz, 1H), 7.40 (d, J=7.0 Hz, 1H), 7.35 (s, 1H), 7.27 (dd, J=9.4, 2.2 Hz, 1H), 7.16-7.11 (m, 2H), 4.73-4.65 (m, 2H), 4.60 (d, J=9.4 Hz, 2H), 4.49 (t, J=7.1 Hz, 1H), 4.43-4.20 (m, 7H), 3.25-3.03 (m, 10H), 2.73-2.64 (m, 6H), 2.46 (s, 3H), 2.19 (s, 3H), 2.10-2.06 (m, 1H), 2.00-1.85 (m, 4H), 1.50-1.42 (m, 2H), 1.40-1.33 (m, 2H), 1.22 (dd, J=8.2, 3.0 Hz, 2H), 0.97 (s, 9H).

### Example A186. 2-amino-3-cyclopropyl-N-((S)-6-(6-(4-((1-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

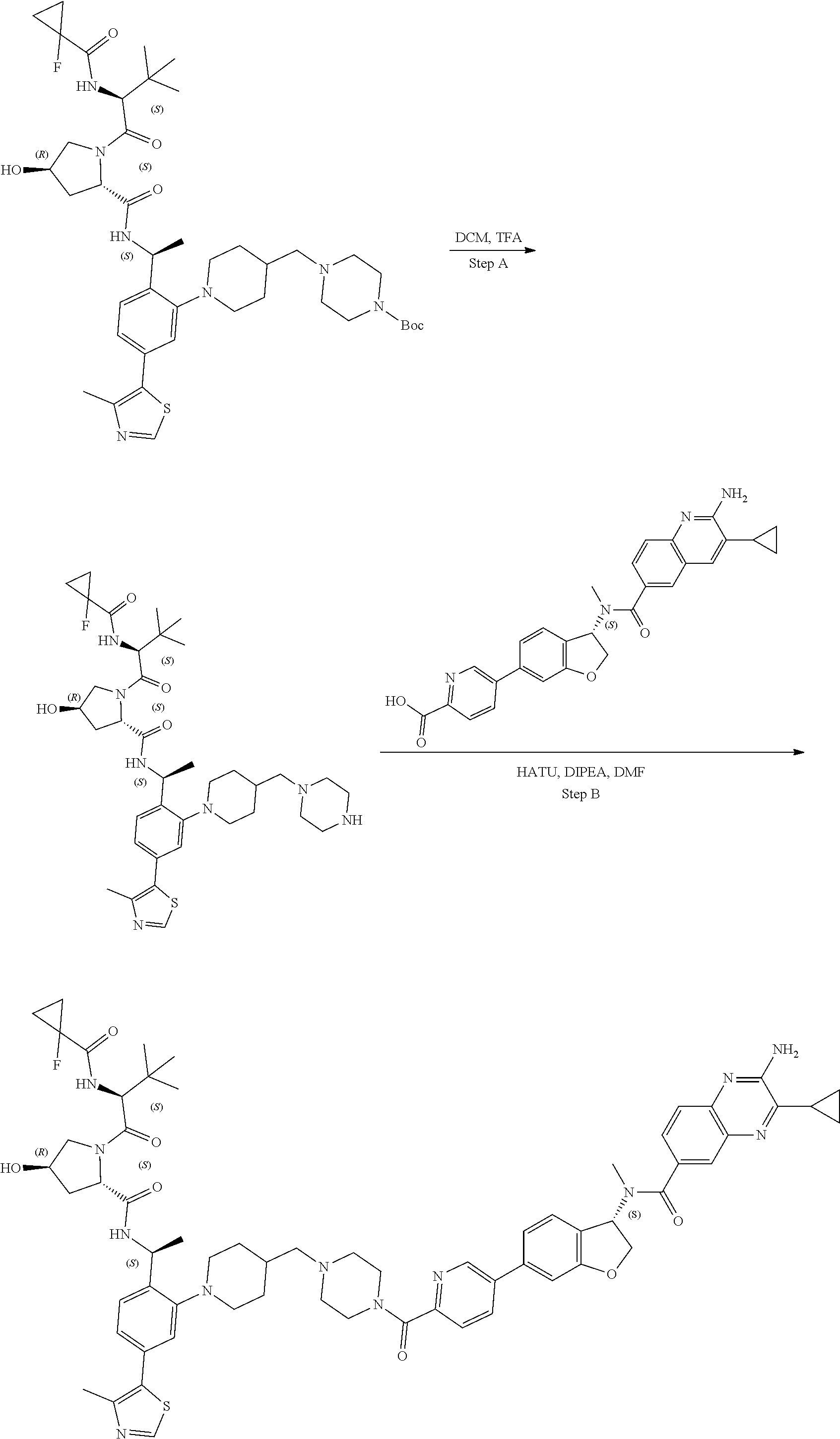

Example A186 was synthesized following the same procedures as described in Example A122 (12.7 mg, 1% yield over 7 steps) as a white solid. LC-MS (ESI): mass calcd. for C₆₅H₇₆FN₁₁O₇S, 1173.6/z found, 1174.6 [M+H]⁺. ¹H NMR (400 MHz, DMSO-d₆) δ 9.59 (s, 1H), 9.00 (s, 1H), 8.94 (s, 2H), 8.34 (d, J=7.9 Hz, 1H), 8.31-8.26 (m, 1H), 8.22 (s, 1H), 8.05 (s, 1H), 7.90-7.84 (m, 1H), 7.78 (d, J=8.2 Hz, 2H), 7.56 (d, J=7.7 Hz, 1H), 7.46-7.35 (m, 3H), 7.28-7.21 (m, 2H), 7.19 (s, 1H), 6.40 (s, 1H), 5.37-5.29 (m, 1H), 4.86-4.78 (m, 1H), 4.71-4.68 (m, 1H), 4.59-4.57 (m, 1H), 4.48-4.46 (m, 1H), 4.31-4.26 (m, 4H), 3.73-3.45 (m, 6H), 3.35-3.27 (m, 2H), 3.22-3.10 (m, 4H), 3.02-2.91 (m, 2H), 2.66 (s, 3H), 2.48 (s, 3H), 2.06-1.87 (m, 4H), 1.83-1.73 (m, 2H), 1.52-1.35 (m, 4H), 1.32 (d, J=6.7 Hz, 3H), 1.25-1.19 (m, 2H), 1.12-1.06 (m, 2H), 0.97 (s, 9H), 0.85-0.79 (m, 2H).

### Example A187. 4-amino-N-((S)-6-(6-(((1S,4S)-4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino)cyclohexyl)carbamoyl) pyridin-3-yl)-2,3-dihydrobenzofuran-3-yl)-N,1-dimethyl-1H-pyrazolo[4,3-c]quinoline-8-carboxamide

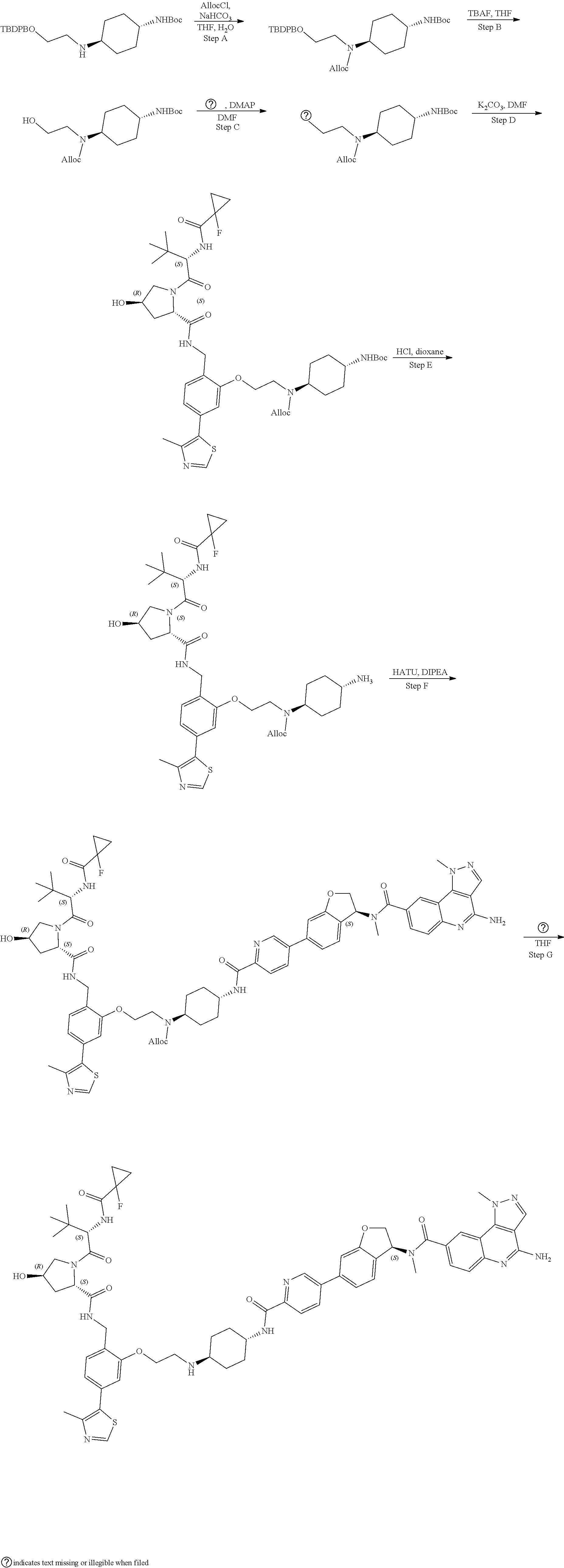

Step A: allyl ((1r,4r)-4-((tert-butoxycarbonyl)amino)cyclohexyl)(2-((tert-butyldiphenylsilyl)oxy)ethyl)carbamate

To a solution of tert-butyl ((1r,4r)-4-((2-((tert-butyldiphenylsilyl)oxy)ethyl)amino)cyclohexyl)carbamate (1.7 g, 3.42 mmol, 1.0 eq.) in DCM (10 mL) was added chloro (prop-2-en-1-yloxy) methanone (436 μL, 4.11 mmol, 1.2 eq.) and Pyridine (817 μL, 10.3 mmol, 3.0 eq.) under N₂. The mixture was stirred at room temperature for 16 hours to give yellow solution. The resulting solution was diluted with 100 mL of H₂O and extracted with DCM (100 mL×3). The combined organic layers were washed with brine, dried over Na₂SO₄ and concentrated under reduced pressure. The residue was purified by flash chromatography (100-200 mesh silica gel, 0%-100% EtOAc in PE) to afford allyl ((1r,4r)-4-((tert-butoxycarbonyl)amino)cyclohexyl)(2-((tert-butyldiphenylsilyl)oxy)ethyl)carbamate (500 mg, 24% yield) as a yellow oil. LC-MS (ESI): mass calcd, for C33H48N205Si, 580.8; m/z found, 525.8 [M+H−56]⁺.

Step B: allyl ((1r,4r)-4-((tert-butoxycarbonyl)amino)cyclohexyl)(2-hydroxyethyl)carbamate

To a solution of allyl ((1r,4r)-4-((tert-butoxycarbonyl)amino)cyclohexyl)(2-((tert-butyldiphenylsilyl)oxy)ethyl)carbamate (500 mg, 861 μmol, 1.0 eq.) in THF (10 mL) was added TBAF (0.86 mL, 1M in THF, 861 μmol, 1.0 eq.) under N₂. The mixture was stirred at room temperature for 1 hour to give yellow solution. The mixture was concentrated. The residue was purified by flash chromatography (100-200 mesh silica gel, 0%-100% EtOAc in PE) to afford allyl ((1r,4r)-4-((tert-butoxycarbonyl)amino)cyclohexyl)(2-hydroxyethyl)carbamate (250 mg, 81%, yield) as a yellow solid. LC-MS (ESI): mass calcd, for C₁₇H₃₀N₂O₅, 342.4; m/z found, 287.4 [M+H−56]⁺.

Step C-F: allyl ((1S,4S)-4-(5-((S)-3-(4-amino-N,1-dimethyl-1H-pyrazolo[4,3-c]quinoline-8-carboxamido)-2,3-dihydrobenzofuran-6-yl)picolinamido)cyclohexyl)(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)carbamate

Example A187 was synthesized following the same procedures as described in Example A181 (7.4 mg, 17% yield over 2 steps) as a white solid.

Step G: 4-amino-N-((S)-6-(6-(((15, 4S)-4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino)cyclohexyl)carbamoyl)pyridin-3-yl)-2,3-dihydrobenzofuran-3-yl)-N,1-dimethyl-1H-pyrazolo[4,3-c]quinoline-8-carboxamide

To a solution of allyl ((1S,4S)-4-(5-((S)-3-(4-amino-N,1-dimethyl-1H-pyrazolo[4,3-c]quinoline-8-carboxamido)-2,3-dihydrobenzofuran-6-yl)picolinamido)cyclohexyl)(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)carbamate (10 mg, 8.11 μmol, 1.0 eq.) in DMF (2 mL) was added Meldrum's acid (2.34 mg, 16.2 μmol, 2.0 eq.), Triethylsilane (2.59 μL, 16.2 μmol, 2.0 eq.), Bis-(triphenylphosphino)-palladous chloride (1.14 mg, 1.62 μmol, 0.2 eq.) and DIEA (2.82 μL, 16.2 μmol, 2 eq.) under N₂. The mixture was stirred at room temperature for 1 hour to give yellow solution. The resulting solution was diluted with 30 mL of H₂O and extracted with EtOAc (30 mL×3). The combined organic layers were washed with brine, dried over Na₂SO₄ and concentrated under reduced pressure. The residue was purified by Prep-HPLC (C18, 5~95%, MeCN in H₂O with 0.1% HCOOH) to afford 4-amino-N-((S)-6-(6-(((1S,4S)-4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino)cyclohexyl)carbamoyl)pyridin-3-yl)-2,3-dihydrobenzofuran-3-yl)-N,1-dimethyl-1H-pyrazolo[4,3-c]quinoline-8-carboxamide (1 mg, 10% yield) a yellow solid. LC-MS (ESI): mass calcd, for C₆₁H₆₉FN₁₂O₈S, 1148.5/z found, 1149.5 [M+H]⁺. ¹H NMR (400 MHz, DMSO-d₆) δ 13.65-13.44 (m, 1H), 9.01 (s, 1H), 8.93 (s, 1H), 8.66-8.58 (m, 5H), 8.46 (s, 1H), 8.28 (d, J=7.5 Hz, 1H), 8.09 (d, J=8.2 Hz, 1H), 7.91 (t, J=12.8 Hz, 1H), 7.62 (s, 1H), 7.42-7.30 (m, 3H), 7.29 (d, J=6.8 Hz, 1H), 7.09-7.00 (m, 2H), 4.87-4.70 (m, 1H), 4.61-4.58 (m, 1H), 4.55-4.41 (m, 5H), 4.39-4.25 (m, 4H), 3.91-3.77 (m, 1H), 3.74-3.55 (m, 2H), 3.51-3.42 (m, 2H), 3.22-3.10 (m, 2H), 2.86-2.55 (m, 4H), 2.47 (s, 3H), 2.24-2.15 (m, 2H), 2.01-1.88 (m, 4H), 1.68-1.49 (m, 4H), 1.42-1.30 (m, 2H), 1.22-1.19 (m, 2H), 0.94 (s, 9H).

### Example A188. 4-amino-3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-1H-pyrazolo[4,3-c]quinoline-8-carboxamide

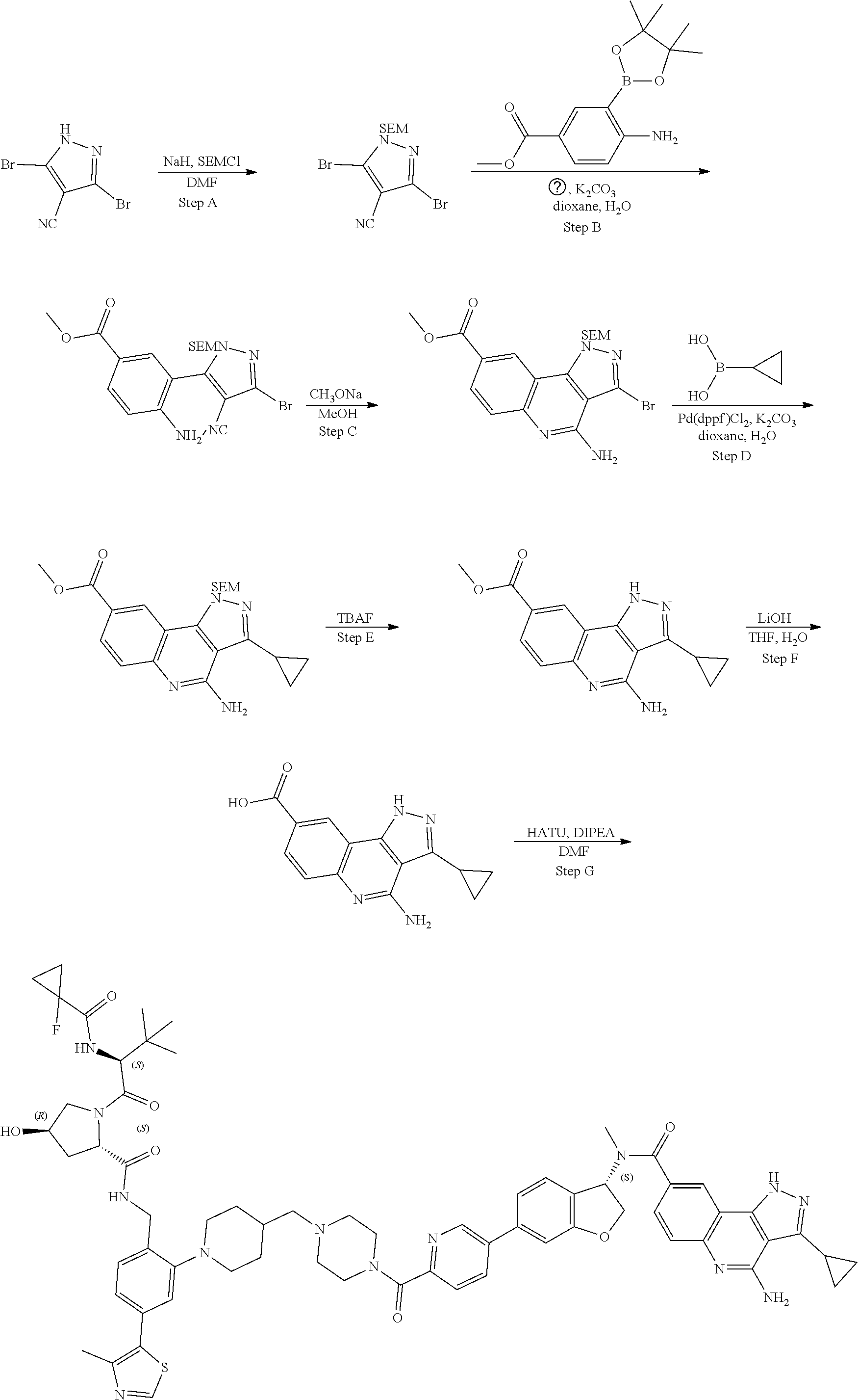

Step A: 3,5-dibromo-1-((2-(trimethylsilyl)ethoxy)methyl)-1H-pyrazole-4-carbonitrile

To a solution of 3,5-dibromo-1H-pyrazole-4-carbonitrile (2 g, 7.9 mmol, 1.0 eq.) in DMF (30 mL) was added NaH (383 mg, 60% Wt, 9.57 mmol, 1.2 eq.) at room temperature. The mixture was at stirred room temperature for 30 mins under nitrogen. Then (2-(chloromethoxy)ethyl) trimethylsilane (1.99 g, 12.0 mmol, 1.5 eq.) was added. The mixture was stirred at room temperature for 4 hours. The resulting solution was diluted with 50 mL of aq. brine (50 mL), dried over Na₂SO₄ and concentrated under reduced pressure. The crude product was purified by flash column chromatography on silica gel (100-200 mesh silica gel, 0~10% EtOAc in PE) to afford 3,5-dibromo-1-((2-(trimethylsilyl)ethoxy)methyl)-1H-pyrazole-4-carbonitrile (2 g, 66% yield) as a white solid. LC-MS (ESI): mass calcd. For C₁₀H₁₅Br₂N₃Osi, 378.94; m/z found, 340 [M+H]⁺.

Step B: methyl 4-amino-3-(3-bromo-4-cyano-1-((2-(trimethylsilyl)ethoxy)methyl)-1H-pyrazol-5-yl)benzoate

A mixture of methyl 4-amino-3-(4,4,5,5-tetramethyl-1,3,2-dioxaborolan-2-yl)benzoate (800 mg, 2.9 mmol, 1.0 eq.), 3,5-dibromo-1-((2-(trimethylsilyl)ethoxy)methyl)-1H-pyrazole-4-carbonitrile (1.3 g, 3.46 mmol, 1.2 eq.), Pd(dppf)Cl₂ (236 mg, 289 μmol, 0.1 eq.) and K₂CO₃ (1.2 g, 8.7 mmol, 3.0 eq.) in 1,4-Dioxane (5 mL) and H₂O (0.5 mL) was stirred at 50° C. for 3 hours under nitrogen. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~35% EtOAc in PE) to afford methyl 4-amino-3-(3-bromo-4-cyano-1-((2-(trimethylsilyl)ethoxy)methyl)-1H-pyrazol-5-yl)benzoate (700 mg, 54% yield) as a yellow solid. LC-MS (ESI): mass calcd. For C₁₈H23BrN₄O₃Si, 450.07; m/z found, 451 [M+H]⁺.

Step C: methyl 4-amino-3-bromo-1-((2-(trimethylsilyl)ethoxy)methyl)-1H-pyrazolo[4,3-c]quinoline-8-carboxylate

A mixture of methyl 4-amino-3-(3-bromo-4-cyano-1-((2-(trimethylsilyl)ethoxy)methyl)-1H-pyrazol-5-yl)benzoate (700 mg, 1.6 mmol, 1.0 eq.) and MeONa (388 mg, 2.3 mmol, 1.5 eq.) in MeOH (10 mL) was stirred at 50° C. for 16 hours under nitrogen. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~100% EtOAc in PE) to afford methyl 4-amino-3-bromo-1-((2-(trimethylsilyl)ethoxy)methyl)-1H-pyrazolo[4,3-c]quinoline-8-carboxylate (330 mg, 47% yield) as a white solid. LC-MS (ESI): mass calcd. For C₁₈H23BrN₄O₃Si, 450.07; m/z found, 451 [M+H]⁺.

Step D: methyl 4-amino-3-cyclopropyl-1-((2-(trimethylsilyl)ethoxy)methyl)-1H-pyrazolo[4,3-c]quinoline-8-carboxylate

A mixture of methyl 4-amino-3-bromo-1-((2-(trimethylsilyl)ethoxy)methyl)-1H-pyrazolo[4,3-c]quinoline-8-carboxylate (80 mg, 177 μmol, 1.0 eq.), cyclopropylboronic acid (30 mg, 0.35 mmol, 2.0 eq.), Pd(dppf)Cl₂ (4 mg, 18 μmol, 0.1 eq.) and K₂CO₃ (74 mg, 0.5 mmol, 3.0 eq.) in 1,4-Dioxane (1 mL) and H₂O (0.1 mL) was stirred at 100° C. for 16 hours under nitrogen. The resulting solution was diluted with 50 mL of water and extracted with EtOAc (20 mL×3). The combined organic layers were washed with brine (30 mL), dried over Na₂SO₄ and concentrated under reduced pressure. The crude product was purified by flash column chromatography on silica gel (100-200 mesh silica gel, 0~100% EtOAc in PE) to afford methyl 4-amino-3-cyclopropyl-1-((2-(trimethylsilyl)ethoxy)methyl)-1H-pyrazolo[4,3-c]quinoline-8-carboxylate (20 mg, 73% yield) as a yellow solid. LC-MS (ESI): mass calcd. For C₂₁H₂₈N₄O₃Si, 412.19; m/z found, 413 [M+H]⁺.

Step E: methyl 4-amino-3-cyclopropyl-1H-pyrazolo[4,3-c]quinoline-8-carboxylate

To a solution of methyl 4-amino-3-cyclopropyl-1-((2-(trimethylsilyl)ethoxy)methyl)-1H-pyrazolo[4,3-c]quinoline-8-carboxylate (40 mg, 97.0 μmol, 1.0 eq.) in DCM (2 mL) was added TFA (1 mL) at room temperature. The mixture was stirred at room temperature for 1 hour under nitrogen. The resulting solution was removed under reduced pressure and adjust to PH=7 with Ammonia water. The crude product was purified by flash column chromatography on silica gel (100-200 mesh silica gel, 0~50% EtOAc in PE) to afford methyl 4-amino-3-cyclopropyl-1H-pyrazolo[4,3-c]quinoline-8-carboxylate (20 mg, 73% yield) as a yellow solid. LC-MS (ESI): mass calcd. For C₁₅H14N402, 282.11; m/z found, 283 [M+H]⁺.

Step F-G: 4-amino-3-cyclopropyl-N-(S)-6-(6-(4-((1-(2-((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-1H-pyrazolo[4,3-c]quinoline-8-carboxamide

Example A188 was synthesized following the same procedures as described in Example A41 (3.9 mg, 7% yield over 2 steps) as a white solid. LC-MS (ESI): mass calcd. for C₆₅H₇₄FN₁₃O₇S, 1199.55; m/z found, 1201 [M+H]⁺. ¹H NMR (400 MHz, DMSO-d₆) δ 9.62 (s, 1H), 8.99 (s, 1H), 8.94 (s, 1H), 8.51 (d, J=5.7 Hz, 1H), 8.40 (s, 1H), 8.29 (d, J=8.3 Hz, 1H), 7.88 (d, J=6.2 Hz, 1H), 7.79 (d, J=8.1 Hz, 2H), 7.58 (d, J=6.7 Hz, 1H), 7.42 (dd, J=20.9, 15.5 Hz, 4H), 7.27 (d, J=6.8 Hz, 1H), 7.13 (d, J=4.8 Hz, 2H), 4.90-4.68 (m, 2H), 4.60 (d, J=9.4 Hz, 2H), 4.52-4.47 (m, 1H), 4.44-4.26 (m, 4H), 3.49-3.44 (m, 2H), 3.38-3.28 (m, 2H), 3.21 -3.06 (m, 7H), 2.76-2.67 (m, 5H), 2.49-2.43 (m, 5H), 2.13-2.07 (m, 1H), 2.06-1.98 (m, 1H), 1.97-1.80 (m, 4H), 1.52-1.42 (m, 2H), 1.41-1.33 (m, 2H), 1.24-1.20 (m, 2H), 1.17-1.10 (m, 2H), 0.98 (s, 9H).

### Example A189. 4-amino-N-((S)-6-(6-(4-((1-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,3-dimethylimidazo[1,5-a]quinoxaline-8-carboxamide

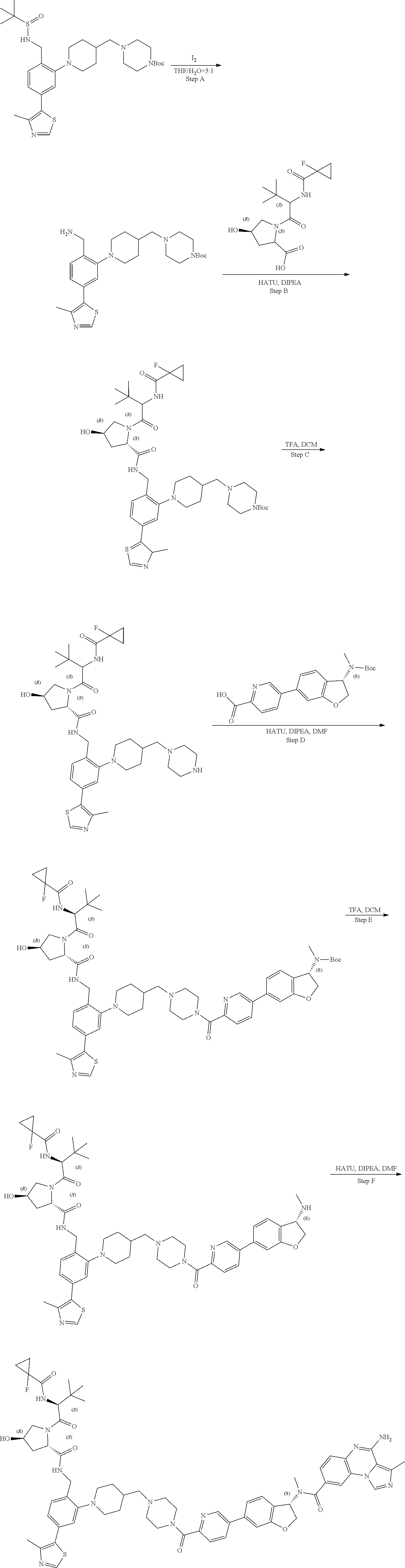

Example A189 was synthesized following the same procedures as described in Example A107 (10.5 mg, 2% yield over 6 steps) as a white solid. LC-MS (ESI): mass calcd. for C₆₃H₇₂FN₁₃O₇S, 1173.54; m/z found, 1174.1 [M+H]⁺. ¹H NMR (400 MHz, DMSO-d₆) δ 9.62 (s, 1H), 9.36 (s, 1H), 8.99 (s, 1H), 8.94 (s, 1H), 8.57-8.42 (m, 2H), 8.29 (d, J=6.9 Hz, 1H), 7.78 (d, J=8.2 Hz, 1H), 7.67-7.60 (m, 2H), 7.57 (d, J=7.7 Hz, 1H), 7.47-7.39 (m, 2H), 7.37 (s, 1H), 7.27 (dd, J=9.2, 2.6 Hz, 1H), 7.16-7.09 (m, 2H), 4.89-4.80 (m, 1H), 4.73-4.65 (m, 2H), 4.60 (d, J=9.2 Hz, 2H), 4.52-4.46 (m, 2H), 4.39-4.33 (m, 4H), 3.71-3.47 (m, 6H), 3.37-3.29 (m, 1H), 3.22-3.07 (m, 6H), 2.72 (s, 3H), 2.69 (s, 3H), 2.46 (s, 3H), 2.13-2.05 (m, 1H), 2.04-1.96 (m, 1H), 1.94-1.83 (m, 3H), 1.51-1.41 (m, 2H), 1.41-1.33 (m, 2H), 1.24-1.20 (m, 2H), 0.97 (s, 9H).

### Example A190. 2-amino-3-cyano-N-((S)-6-(6-(4-((1-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenyl)piperidin-4-yl)methyl)piperazine-1-carbonyl)pyridin-3-yl)~2,3-dihydrobenzofuran-3-yl)-N-methylquinoline-6-carboxamide

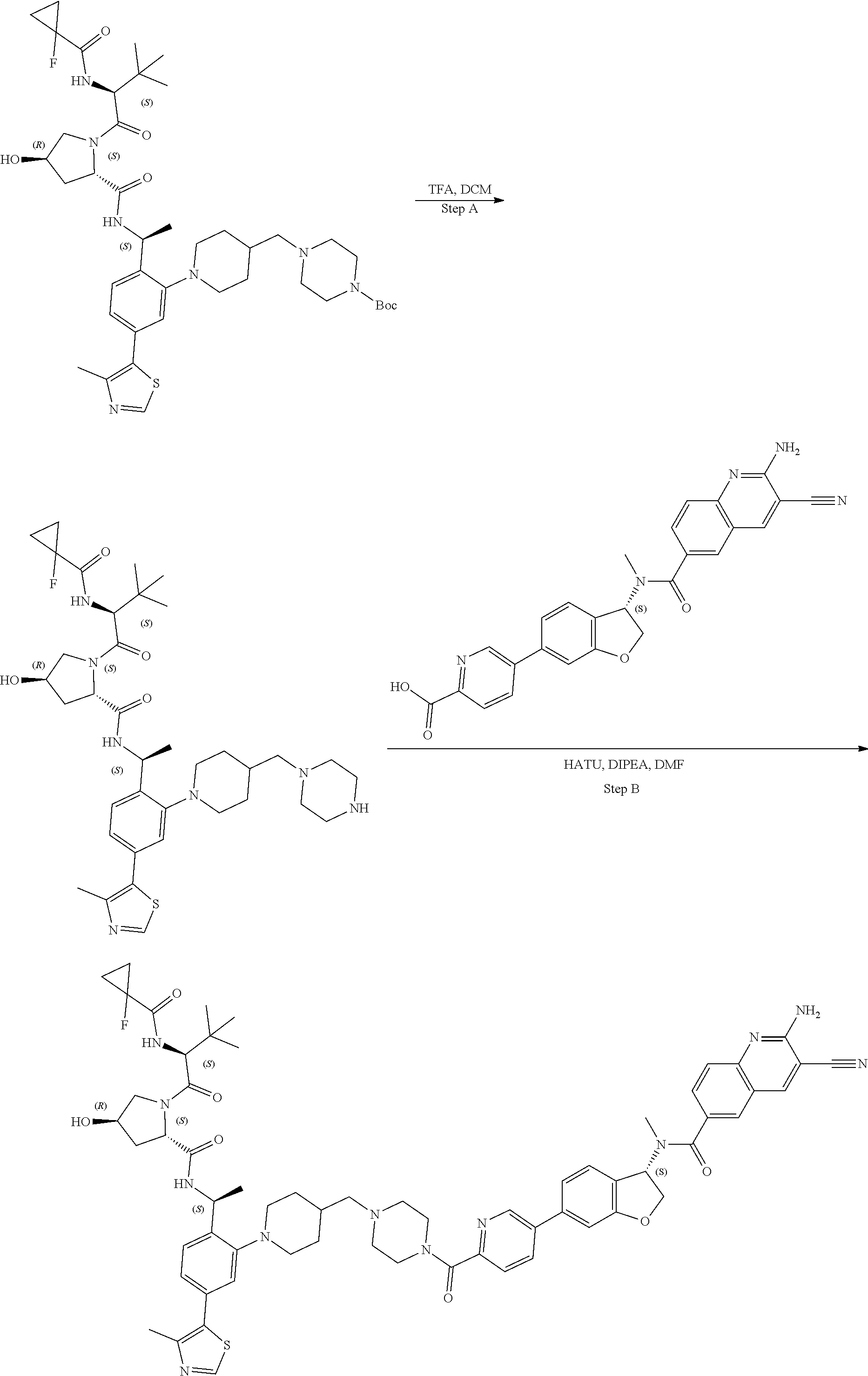

Example A190 was synthesized following the same procedures as described in Example A107 (4.3 mg, 7% yield over 2 steps) as a white solid. LC-MS (ESI): mass calcd. for C₆₃H₇₁FN₁₂O₇S, 1158.5/z found, 1159.2 [M+H]⁺. ¹H NMR (400 MHz, DMSO-d₆) δ 9.43 (s, 1H), 9.00 (s, 1H), 8.93 (d, J=1.8 Hz, 1H), 8.80 (s, 1H), 8.40-8.23 (m, 2H), 7.96 (s, 1H), 7.84-7.74 (m, 2H), 7.62-7.54 (m, 2H), 7.45 (d, J=8.0 Hz, 1H), 7.40 (d, J=7.9 Hz, 1H), 7.35 (s, 1H), 7.31 (s, 1H), 7.28-7.21 (m, 2H), 7.18 (s, 1H), 6.59 (s, 1H), 5.38-5.29 (m, 1H), 4.80-4.68 (m, 2H), 4.65-4.55 (m, 2H), 4.49-4.44 (m, 1H), 4.32-4.23 (m, 2H), 3.65-3.48 (m, 7H), 3.35-3.27 (m, 2H), 3.23-3.10 (m, 4H), 3.02-2.90 (m, 2H), 2.68 (s, 3H), 2.48 (s, 3H), 2.07-2.00 (m, 1H), 1.99-1.85 (m, 2H), 1.84-1.69 (m, 2H), 1.55-1.34 (m, 4H), 1.31 (d, J=6.7 Hz, 3H), 1.25-1.18 (m, 2H), 0.96 (s, 9H).

### Example A192. 2-amino-3-cyclopropyl-N-((3S)-6-(6-(5-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl) octahydro-pyrrolo[3,4-c]pyrrole-2-carbonyl)pyridin-3-yl)-2,3-dihydrobenzofuran-3-yl)-N-methylquinoline-6-carboxamide

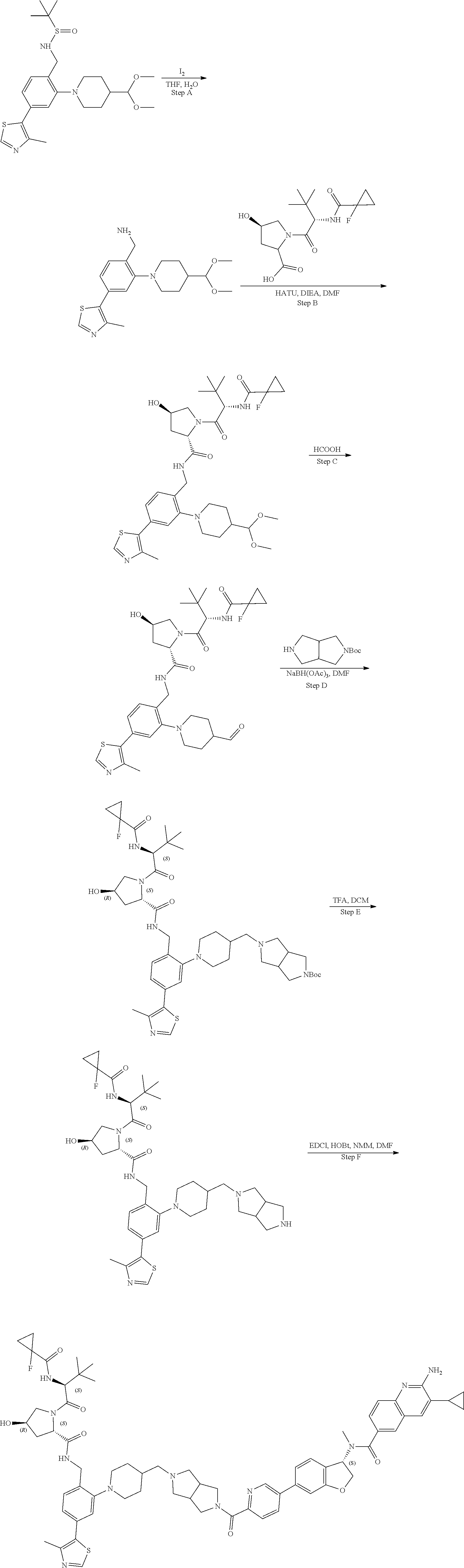

Example A192 was synthesized following the same procedures as described in Example A107 (3.6 mg, 3% yield over 6 steps) as a white solid. LC-MS (ESI): mass calcd. for C₆₆H₇₆FN₁₁O₇S, 1185.56; m/z found, 1187.0 [M+H]⁺. ¹HNMR (400 MHz, DMSO-d₆) δ 9.62 (s, 1H), 8.99 (s, 1H), 8.93 (s, 1H), 8.86 (s, 1H), 8.51 (t, J=5.6 Hz, 1H), 8.29-8.23 (m, 1H), 8.21 (s, 1H), 8.05 (s, 1H), 7.86 (t, J=7.6 Hz, 2H), 7.76 (d, J=7.0 Hz, 1H), 7.55 (d, J=7.6 Hz, 1H), 7.44 (d, J=8.3 Hz, 1H), 7.40 (d, J=6.8 Hz, 1H), 7.35 (s, 1H), 7.26 (dd, J=8.9, 2.3 Hz, 1H), 7.14-7.11 (m, 2H), 4.92-4.74 (m, 1H), 4.68 (d, J=6.6 Hz, 1H), 4.60 (d, J=9.2 Hz, 1H), 4.49 (t, J=8.2 Hz, 1H), 4.42-4.28 (m, 4H), 3.70-3.58 (m, 5H), 3.28-3.01 (m, 8H), 2.79-2.57 (m, 6H), 2.46 (s, 3H), 2.12-2.06 (m, 1H), 1.95-1.79 (m, 6H), 1.50-1.30 (m, 5H), 1.22 (dd, J=8.3, 2.6 Hz, 2H), 1.08 (dd, J=8.2, 1.6 Hz, 2H), 0.97 (s, 9H), 0.81 (d, J=3.8 Hz, 2H).

### Example A193. 2-amino-3-cyclopropyl-N-((S)-6-(6-(6-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,6-diazaspiro[3.3]heptane-2-carbonyl)pyridin-3-yl)-2,3-dihydrobenzofuran-3-yl)-N-methylquinoline-6-carboxamide

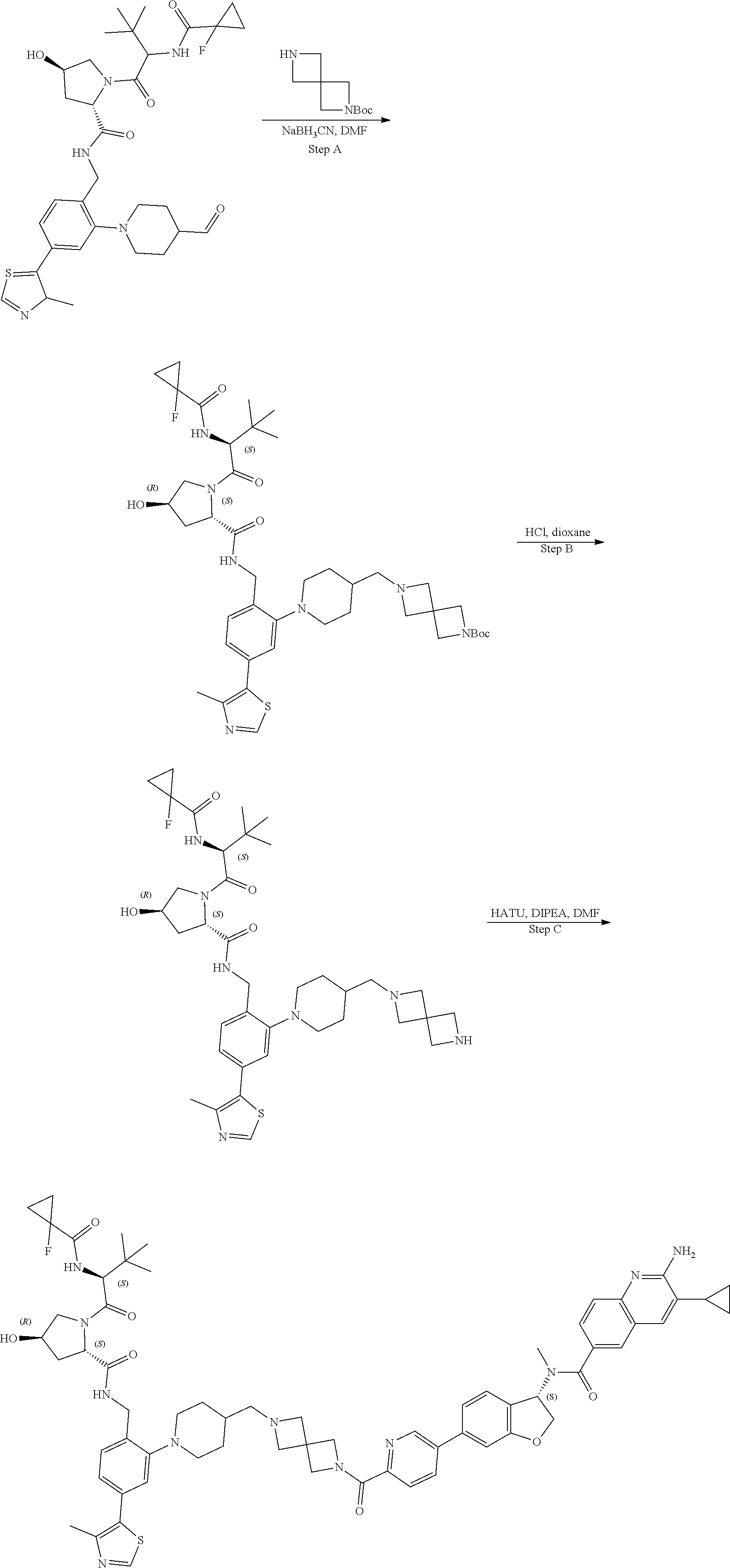

Example A193 was synthesized following the same procedures as described in Example A107 (6.2 mg, 6% yield over 3 steps) as a white solid. LC-MS (ESI): mass calcd. for C₆₅H₇₄FN₁₁O₇S, 1171.54/z found, 1172.6 [M+H]⁺, ¹H NMR (400 MHz, DMSO-d₆) δ 9.71 (s, 1H), 8.99 (s, 1H), 8.94 (d, J=7.1 Hz, 2H), 8.51 (t, J=5.7 Hz, 1H), 8.26 (dd, J=8.2, 1.9 Hz, 1H), 8.21 (s, 1H), 8.03 (d, J=8.4 Hz, 2H), 7.86 (dd, J=8.7, 1.3 Hz, 1H), 7.76 (s, 1H), 7.55 (d, J=7.7 Hz, 1H), 7.45-7.38 (m, 2H), 7.36 (s, 1H), 7.27 (dd, J=9.4, 2.5 Hz, 1H), 7.13 (d, J=6.7 Hz, 2H), 4.88 (s, 1H), 4.78 (s, 1H), 4.68 (d, J=6.4 Hz, 2H), 4.60 (d, J=9.3 Hz, 1H), 4.52-4.46 (m, 2H), 4.46-4.40 (m, 3H), 4.37-4.33 (m, 4H), 4.31-4.25 (m, 4H), 3.67-3.60 (m, 2H), 3.21-3.13 (m, 2H), 3.11-3.05 (m, 2H), 3.11-3.04 (m, 2H), 2.69-2.63 (m, 4H), 2.46 (s, 3H), 2.12-2.06 (m, 1H), 1.94-1.88 (m, 2H), 1.78-1.67 (m, 3H), 1.43-1.31 (m, 4H), 1.25-1.19 (m, 2H), 1.10-1.05 (m, 2H), 0.97 (s, 9H), 0.81 (d, J=4.0 Hz, 2H).

### Example A194. 4-amino-N-((S)-6-(6-(4-((1-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenyl)piperidin-4-yl)methyl)piperazine-1-carbonyl)pyridin-3-yl)-2,3-dihydrobenzofuran-3-yl)-N,1,3-trimethyl-1H-pyrazolo[4,3-c]quinoline-8-carboxamide

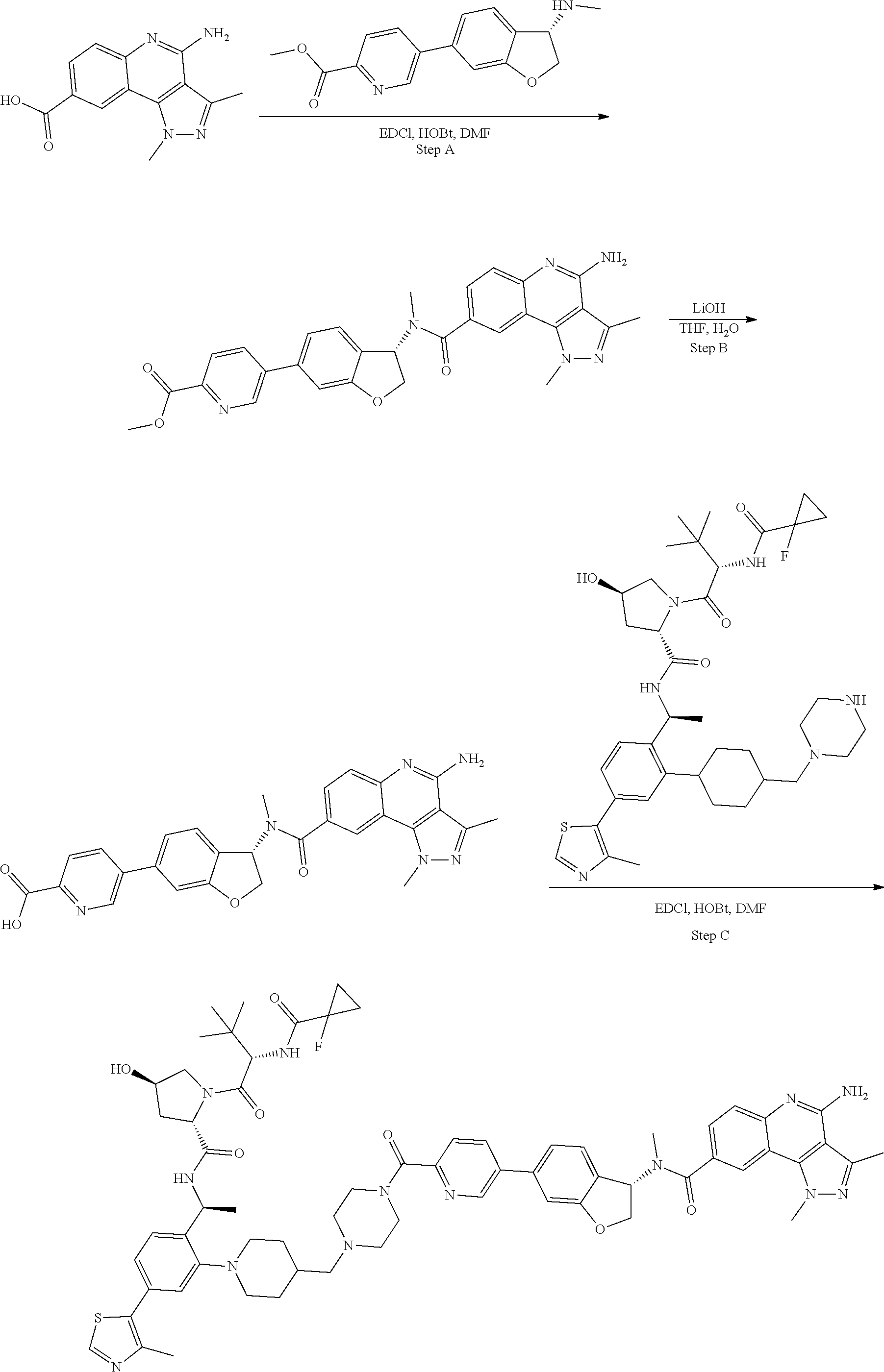

Example A194 was synthesized following the same procedures as described in Example A41 (3.2 mg, 7.2% yield over 3 steps) as a white solid. LC-MS (ESI): mass calcd. for C₆₅H₇₆FN₁₃O₇S, 1201.57 m/z found, 1202.2 [M+H]⁺. ¹H NMR (400 MHz, DMSO-d₆) δ 9.70 (s, 1H), 8.99 (s, 1H), 8.93 (s, 1H), 8.79 (s, 1H), 8.46-8.34 (m, 2H), 8.28 (m, 1H), 7.93 (m, 1H), 7.86 (s, 1H), 7.78 (m, 1H), 7.59 (m, 2H), 7.41 (m, 3H), 7.23 (m, 1H), 7.20-7.15 (m, 2H), 5.30-5.23 (m, 1H), 4.77 (s, 2H), 4.57 (t, J=8.4 Hz, 2H), 4.48 (t, J=8.0 Hz, 2H), 4.43 (s, 3H), 4.35 (s, 1H), 4.30-4.21 (m, 1H), 3.35-3.32 (m, 4H), 3.15 (s, 4H), 3.03-2.91 (m, 3H), 2.72-2.67 (m, 8H), 2.47 (s, 3H), 2.09-2.03 (m, 1H), 2.01-1.86 (m, 4H), 1.79-1.73 (m, 1H), 1.53-1.46 (m, 1H), 1.45-1.35 (m, 3H), 1.34-1.27 (m, 5H), 1.25-1.18 (m, 3H), 0.90 (s, 9H).

### Example A196. 4-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylpyrazolo[1,5-a]quinoxaline-8-carboxamide

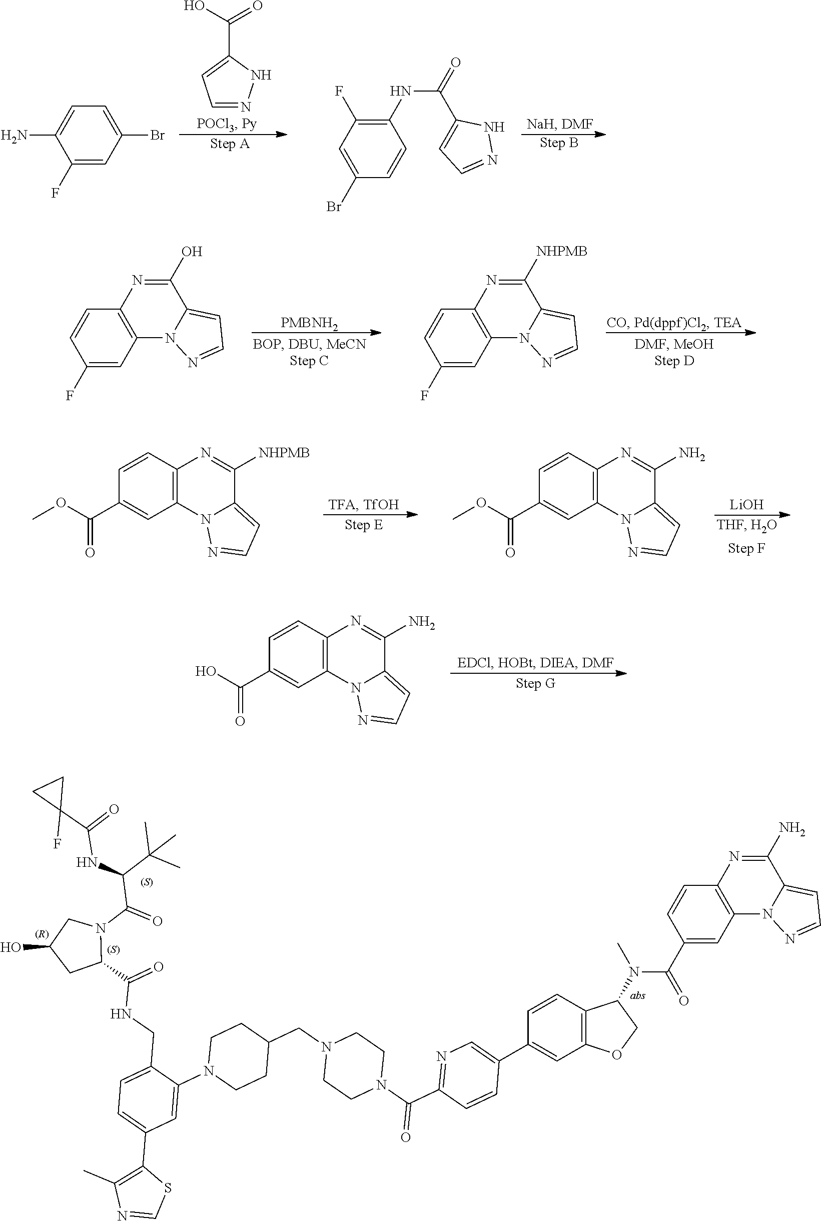

Step A: N-(4-bromo-2-fluorophenyl)-1H-pyrazole-5-carboxamide

To a solution of 4-bromo-2-fluoroaniline (2.00 g, 10.5 mmol, 1.0 eq.) in Py (15 mL) was added 1H-pyrazole-5-carboxylic acid (1.42 g, 12.6 mmol, 1.2 eq.) and Phosphorus oxychloride (1.61 g, 10.5 mmol, 1.0 eq.) at 0° C. The mixture was then stirred at 0° C. for 1 hour under N₂ atmosphere. The resulting solution was diluted with 30 mL of water and extracted with EtOAc (50 mL×2). The combined organic layers were washed with brine, dried over anhydrous Na₂SO₄ and concentrated under reduced pressure. The residue was purified by flash column chromatography on silica gel (100-200 mesh silica gel, 0-30% EtOAc in PE) to afford N-(4-bromo-2-fluorophenyl)-1H-pyrazole-5-carboxamide (1.00 g, 33% yield) as a white solid. LC-MS (ESI): mass calcd. For C₁₀H₇BrFN₃O, 282.98; m/z found, 284.2 [M+H]⁺.

Step B: 8-bromopyrazolo[1,5-a]quinoxalin-4-ol

To a solution of N-(4-bromo-2-fluorophenyl)-1H-pyrazole-5-carboxamide (600 mg, 2.11 mmol, 1.0 eq.) in DMA (10.0 mL) was added NaH (101 mg, 4.22 mmol, 2.0 eq.) at room temperature. The mixture was then stirred at 120° C. for 16 hours under N₂ atmosphere. The resulting solution was diluted with 20 mL of NH₄Cl/H₂O. The precipitate was collected and washed with water (20 mL), then concentrated under reduced pressure to obtain a white solid of 8-bromopyrazolo[1,5-a]quinoxalin-4-ol (300 mg, 54% yield). LC-MS (ESI): mass calcd. For C₁₀H₆BrN₃O, 262.97; m/z found, 264.1 [M+H]⁺.

Step C: 8-bromo-N-(4-methoxybenzyl)pyrazolo[1,5-a]quinoxalin-4-amine

To a solution of 8-bromopyrazolo[1,5-a]quinoxalin-4-ol (200 mg, 757 μmol, 1.0 eq.), (4-methoxyphenyl)methanamine (260 mg, 1.89 mmol, 2.5 eq.) and BOP (670 mg, 1.51 mmol, 2.0 eq.) in MeCN (15 mL) was added DBU (576 mg, 3.79 mmol, 5.0 eq.), the reaction was stirred at 50° C. for 16 hours. The reaction was purified by reverse phase purification (C18, MeCN/H2O, 0.1% FA, 0 to 55% gradient, 30 mins run) and the fractions containing desired product were lyophilized afford the 8-bromo-N-(4-methoxybenzyl)pyrazolo[1,5-a]quinoxalin-4-amine (150 mg, 52% yield) as a yellow solid. LC-MS (ESI): mass calcd. For C₁₈H₁₅BrN₄O, 382.04; m/z found, 383.1 [M+H]⁺.

Step D: methyl 4-((4-methoxybenzyl)amino)pyrazolo[1,5-a]quinoxaline-8-carboxylate

To a solution of 8-bromo-N-(4-methoxybenzyl)pyrazolo[1,5-a]quinoxalin-4-amine (150 mg, 391 μmol, 1.0 eq.) and 1,1′-Bis(diphenylphosphino) ferrocene-palladium (II) dichloride (57.3 mg, 78.3 μmol, 0.2 eq.) in DMF (3 mL) and MeOH (10 mL) was added triethylamine (119 mg, 1.17 mmol, 3.0 eq.), the reaction was stirred at 80° C. for 16 hours under CO. The reaction was purified by reverse phase purification (C18, MeCN/H2O, 0.1% FA, 0 to 55% gradient, 30 min run) and the fractions containing desired product were lyophilized afford the methyl 4-((4-methoxybenzyl)amino)pyrazolo[1,5-a]quinoxaline-8-carboxylate (80.0 mg, 56% yield) as a yellow solid. LC-MS (ESI): mass calcd. For C₂₀H₁₅N₄O₃, 362.14; m/z found, 363.2 [M+H]⁺.

Step E: methyl 4-aminopyrazolo[1,5-a]quinoxaline-8-carboxylate

To a solution of methyl 4-((4-methoxybenzyl)amino)pyrazolo[1,5-a]quinoxaline-8-carboxylate (67.0 mg, 185 μmol, 1.0 eq) in TFA: TfOH=40:1 (4 mL) was added at room temperature. The mixture was then stirred at 60° C. for 1 hour under N₂ atmosphere. The reaction was monitored by LCMS. The resulting solution was diluted with 30 mL of NaHCO₃/H2O and extracted with EtOAc (50 mL×2). The combined organic layers were washed with brine, dried over anhydrous Na₂SO₄, and under reduced pressure to afford methyl 4-aminopyrazolo[1,5-a]quinoxaline-8-carboxylate (44.0 mg, 98% yield) as a yellow solid. LC-MS (ESI): mass calcd. For C₁₂H₁₀N₄O₂, 242.08; m/z found, 243.2 [M+H]⁺.

Step F: 4-aminopyrazolo[1,5-a]quinoxaline-8-carboxylic acid

To a solution of methyl 4-aminopyrazolo[1,5-a]quinoxaline-8-carboxylate (10.0 mg, 41.3 μmol, 1.0 eq.) in THF: H₂O=4:1 (5 mL) was added LiOH (1.98 mg, 82.6 μmol, 2.0 eq.) at room temperature. The mixture was then stirred at 50° C. for 4 hours under N₂. The reaction was concentrated under vacuum to afford 4-aminopyrazolo[1,5-a]quinoxaline-8-carboxylic acid (9.0 mg, 95% crude) as a yellow solid. LC-MS (ESI): mass calcd. For C₁₁H₈N₄O₂, 228.06; m/z found, 229.2 [M+H]⁺.

Step G: 4-amino-N-(S)-6-16-(4-((1-(2-(2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylpyrazolo[1,5-a]quinoxaline-8-carboxamide

Example A196 was synthesized following the same procedures as described in Example A41 (2.6 mg, 11% yield over 1 steps) as a white solid. LC-MS (ESI): mass calcd. for C₆₂H₇₀FN₁₃O₇S, 1159.52 m/z found, 1160.2 [M+H]⁺. ¹H NMR (400 MHz, DMSO-d₆) δ 9.56 (s, 1H), 8.99 (s, 1H), 8.94 (d, J=1.8 Hz, 1H), 8.64-8.50 (m, 2H), 8.36 (s, 1H), 8.29 (dd, J=8.2, 2.2 Hz, 1H), 8.23 (s, 1H), 7.78 (d, J=8.2 Hz, 1H), 7.70-7.58 (m, 3H), 7.45-7.40 (m, 3H), 7.35 (s, 1H), 7.29-7.25 (m, 1H), 7.13 (d, J=5.1 Hz, 2H), 4.78-4.74 (m, 1H), 4.60 (d, J=9.3 Hz, 1H), 4.51-4.47 (m, 1H), 4.43-4.30 (m, 4H), 4.28-4.20 (m, 1H), 3.69-3.60 (m, 4H), 3.55-3.51 (m, 1H), 3.36-3.29 (m, 1H), 3.22-3.06 (m, 7H), 2.76 (d, J=10.0 Hz, 1H), 2.71-2.66 (m, 4H), 2.46 (s, 3H), 2.13-2.06 (m, 1H), 2.05-1.81 (m, 5H), 1.51-1.42 (m, 2H), 1.41-1.38 (m, 1H), 1.36-1.33 (m, 1H), 1.24-1.20 (m, 2H), 0.98 (s, 9H).

### Example A198. 2-amino-3-cyclopropyl-N-((S)-6-(6-(4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)methyl)piperazine-1-carbonyl)pyridin-3-yl)-2,3-dihydrobenzofuran-3-yl)-N-methylquinoline-6-carboxamide

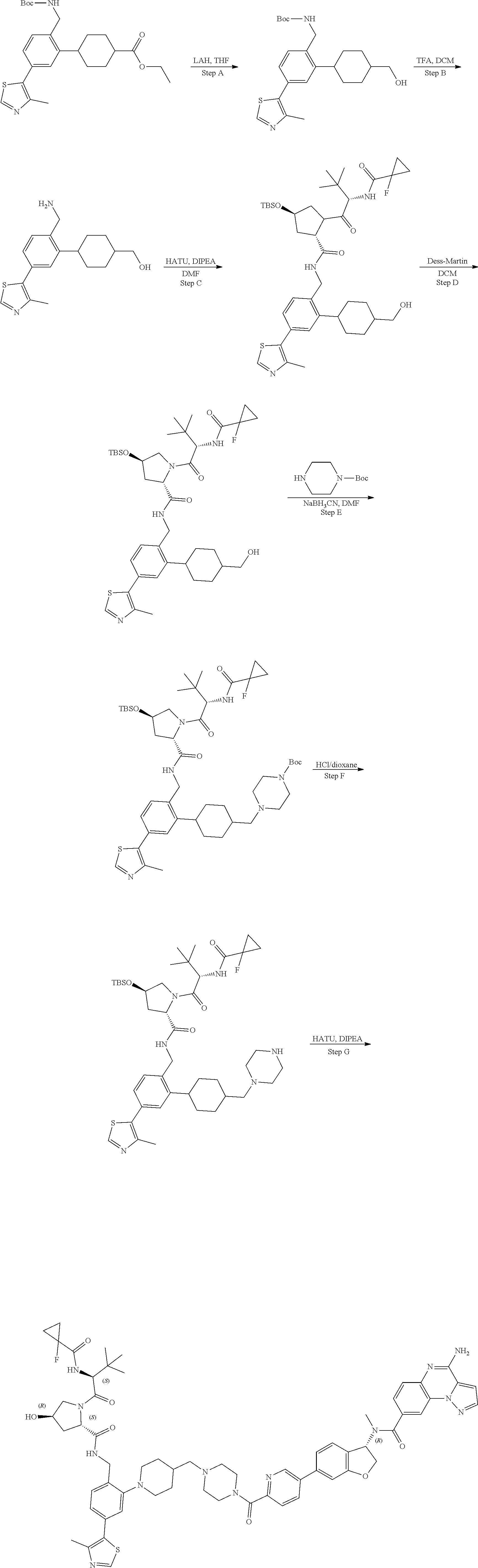

Example A198 was synthesized following the same procedures as described in Example A145 (11.0 mg, 33% yield over 7 steps) as a white solid. LC-MS (ESI): mass calcd. for C₆₅H₇₅FN₁₀O₇S, 1158.55/z found, 1160.0 [M+H]⁺. ¹HNMR (400 MHz, DMSO-d₆) δ 9.59 (s, 1H), 9.01-8.89 (m, 3H), 8.55-8.45 (m, 1H), 8.31-8.26 (m, 1H), 8.22 (s, 1H), 8.05 (s, 1H), 7.89-7.85 (m, 1H), 7.80-7.72 (m, 2H), 7.56 (d, J=7.4 Hz, 1H), 7.46-7.39 (m, 2H), 7.37-7.30 (m, 2H), 7.28-7.22 (m, 2H), 4.88-4.66 (m, 2H), 4.59 (d, J=9.2 Hz, 2H), 4.48 (t, J=8.2 Hz, 1H), 4.41 (dd, J=15.8, 6.3 Hz, 1H), 4.34 (s, 2H), 4.31-4.20 (m, 2H), 3.56-3.29 (m, 6H), 3.23-3.02 (m, 4H), 2.83-2.76 (m, 1H), 2.66 (s, 3H), 2.47-2.45 (m, 3H), 2.10-2.03 (m, 1H), 1.95-1.73 (m, 7H), 1.66-1.49 (m, 3H), 1.42-1.33 (m, 2H), 1.25-1.19 (m, 3H), 1.10-1.06 (m, 2H), 0.99-0.94 (m, 9H), 0.84-0.79 (m, 2H).

### Example A199. 2-amino-3-cyano-7-fluoro-N-((S)-6-(6-(4-((1-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

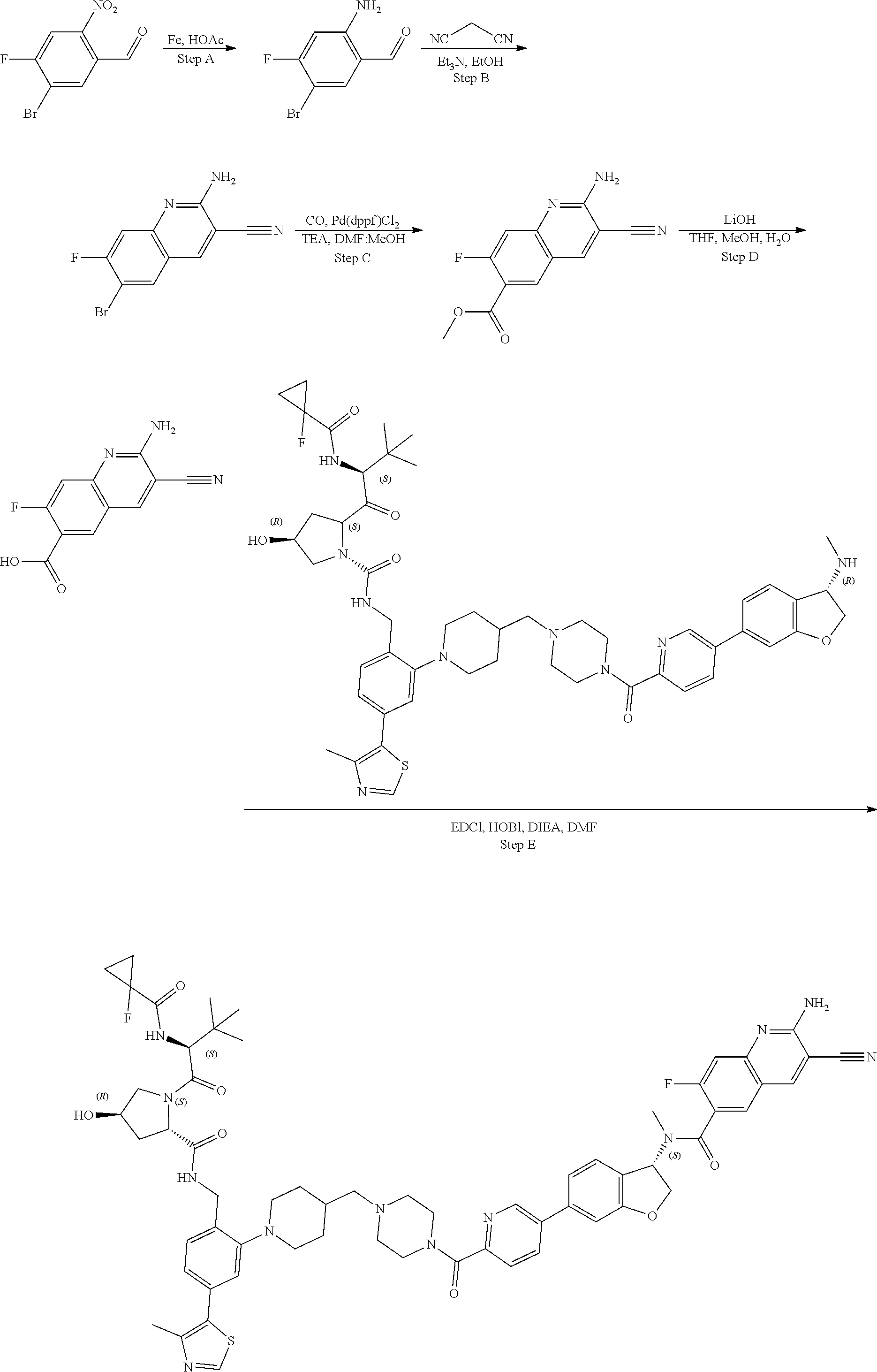

Step A: 2-amino-5-bromo-4-fluorobenzaldehyde

To a solution of 5-bromo-4-fluoro-2-nitrobenzaldehyde (1 g, 4.0 mmol, 1.0 eq.) in EtOH (10 mL) and Water (2 mL) was added AcOH (2.1 mL, 36.0 mmol, 9.0 eq.) and Iron (676 mg, 12 mmol, 3.0 eq.). The resulting mixture was stirred at 80° C. for 1 hour. The resulting mixture was filtered through a pad of celite and added water (40 mL). The mixture was diluted with EtOAc (50 mL×2). The combined organic layers were washed with brine, dried over anhydrous Na₂SO₄ and concentrated. The residue was purified by column chromatography (100-200 mesh silica gel, 0%~10% EtOAc in PE) to afford 2-amino-5-bromo-4-fluorobenzaldehyde (230 mg, 26% yield) as a yellow solid. LC-MS (ESI): mass calcd. For C₇H₅BrFNO, 216.95; m/z found, 218.0 [M+H]⁺.

Step B: 2-amino-6-bromo-7-fluoroquinoline-3-carbonitrile

To a solution of 2-amino-5-bromo-4-fluorobenzaldehyde (180 mg, 826 μmol, 1.0 eq.) in EtOH (5 mL) was added Et₃N (230 μL, 1.6 mmol, 2.0 eq.) and Malononitrile (109 mg, 1.6 mmol, 2.0 eq.). The resulting mixture was stirred at 90° C. for 5 hours. The reaction mixture was poured into water (20 mL) and then extracted with EtOAc (20 mL×2). The combined organic layers were washed with brine, dried over anhydrous Na₂SO₄ and concentrated. The residue was purified by column chromatography (silica gel, 0%~10% MeOH in DCM) to afford 2-amino-6-bromo-7-fluoroquinoline-3-carbonitrile (150 mg, 68% yield) as a yellow solid. LC-MS (ESI): mass calcd. For C₁₀H₅BrFN₃, 264.97; m/z found, 266.0 [M+H]⁺.

Step C: methyl 2-amino-3-cyano-7-fluoroquinoline-6-carboxylate

To a mixture of 2-amino-6-bromo-7-fluoroquinoline-3-carbonitrile (150 mg, 564 μmol, 1.0 eq.) in DMF (3 mL) and MeOH (1 mL) was added TEA (393 μL, 2.8 mmol, 5.0 eq.) and Pd(dppf)Cl₂ (41.3 mg, 56.4 μmol, 0.1 eq.). The reaction mixture was stirred at 80° C. for 16 hours under CO gas. The reaction mixture was poured into water (20 mL) and then extracted with EtOAc (20 mL×2). The combined organic layers were washed with brine, dried over anhydrous Na₂SO₄ and concentrated. The residue was purified by column chromatography (100-200 mesh silica gel, 0%~80% EtOAc in PE) to afford methyl 2-amino-3-cyano-7-fluoroquinoline-6-carboxylate (80 mg, 58% yield) as a yellow solid. LC-MS (ESI): mass calcd. For C₁₂H₈FN₃O₂, 245.06; m/z found, 246.1 [M+H]⁺.

Steps D-E: 2-amino-3-cyano-7-fluoro-N-(S)-6-(6-(4-((1-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

Example A199 was synthesized following the same procedures as described in Example A114 (2.0 mg, 2% yield over 5 steps) as a white solid. LC-MS (ESI): mass calcd. for C₆₂H68F₂N1207S, 1162.50; m/z found, 1164.0 [M+H]⁺. ¹HNMR (400 MHz, DMSO-d₆) δ 9.60 (s, 1H), 8.99 (s, 1H), 8.93 (d, J=6.5 Hz, 1H), 8.76 (d, J=16.4 Hz, 1H), 8.52 (s, 1H), 8.28 (t, J=7.1 Hz, 1H), 7.95 (d, J=7.8 Hz, 1H), 7.78 (dd, J=8.1, 3.6 Hz, 1H), 7.52-7.41 (m, 3H), 7.40-7.32 (m, 4H), 7.29-7.24 (m, 1H), 7.14 (d, J=5.8 Hz, 2H), 4.71-4.56 (m, 4H), 4.51-4.46 (m, 1H), 4.41-4.33 (m, 3H), 4.31-4.11 (m, 2H), 3.38-3.27 (m, 3H), 3.24-3.07 (m, 8H), 2.76-2.70 (m, 3H), 2.69-2.66 (m, 1H), 2.58 (s, 2H), 2.46 (s, 3H), 2.13-2.00 (m, 2H), 1.93-1.84 (m, 3H), 1.51-1.42 (m, 2H), 1.40-1.33 (m, 2H), 1.24-1.20 (m, 2H), 0.98 (s, 9H).

### Example A200. 2-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-3-methoxy-N-methylquinoline-6-carboxamide

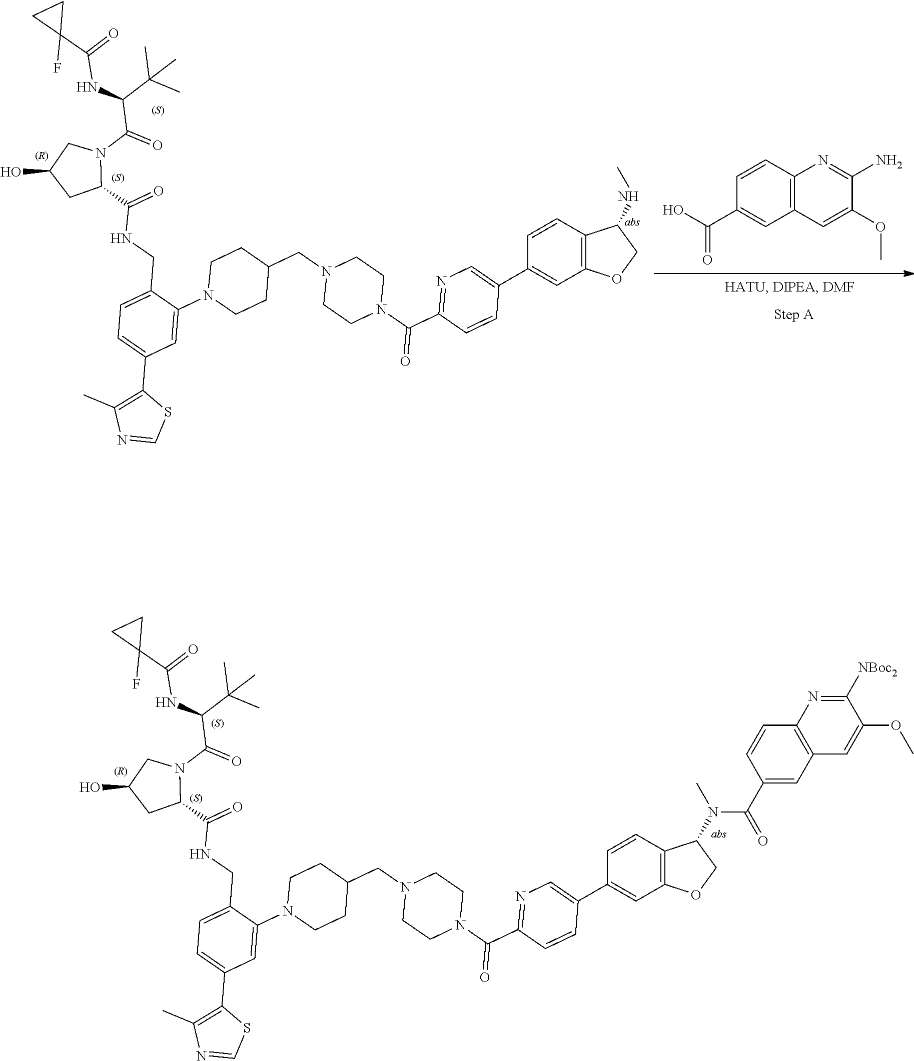

Example A200 was synthesized following the same procedures as described in Example A114 (3.1 mg, yield 12.8%) as a white solid. LC-MS (ESI): mass calcd, for C₆₂H₇₂FN₁₁O₈S, 1149.53; m/z found, 1150.6 [M+H]⁺. ¹H NMR (400 MHz, DMSO-d₆) δ 9.60 (s, 1H), 8.99 (s, 1H), 8.94 (s, 1H), 8.83 (s, 1H), 8.52 (t, J=5.8 Hz, 1H), 8.29 (dd, J=8.2, 2.1 Hz, 1H), 7.99 (s, 1H), 7.86 (s, 1H), 7.78 (d, J=8.2 Hz, 1H), 7.72 (s, 2H), 7.56 (d, J=7.6 Hz, 1H), 7.42 (dd, J=14.2, 8.2 Hz, 2H), 7.36 (s, 1H), 7.27 (d, J=6.8 Hz, 1H), 7.13 (d, J=4.4 Hz, 2H), 4.85-4.66 (m, 2H), 4.65-4.53 (m, 2H), 4.52-4.47 (m, 1H), 4.42-4.23 (m, 4H), 4.04 (s, 3H), 3.73-3.58 (m, 8H), 3.18-3.09 (m, 5H), 2.75-2.65 (m, 5H), 2.46 (s, 3H), 2.12-2.06 (m, 1H), 2.02-1.96 (m, 1H), 1.93-1.83 (m, 3H), 1.51-1.42 (m, 2H), 1.40-1.33 (m, 2H), 1.25-1.20 (m, 2H), 0.97 (d, J=8.8 Hz, 9H).

### Example A201. 2-amino-3-cyclopropyl-N-((S)-6-(6-(4-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidine-4-carbonyl)piperazine-1-carbonyl)pyridin-3-yl)-2,3-dihydrobenzofuran-3-yl)-N-methylquinoline-6-carboxamide

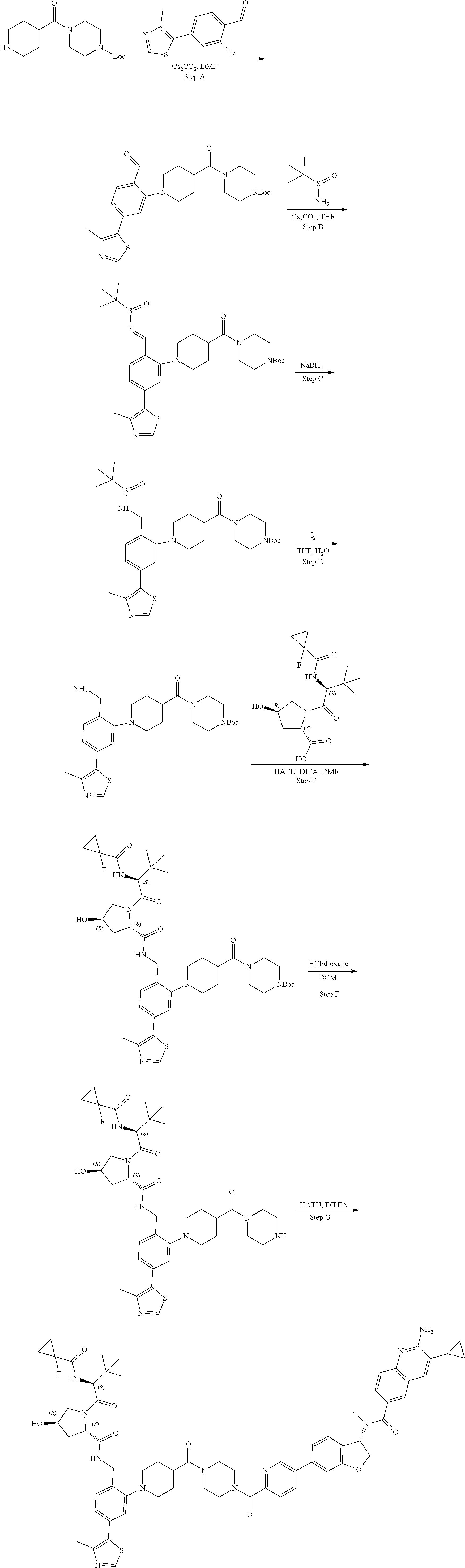

Example A201 was synthesized following the same procedures as described in Example A126 (5.0 mg, 7% yield over 7 steps) as a white solid. LC-MS (ESI): mass calcd. for C64H72FN11O8S, 1173.53; m/z found, 1174.5 [M+H]⁺. ¹H NMR (400 MHz, DMSO) δ 8.99 (d, J 6.5 Hz, 1H), 8.93 (s, 1H), 8.83 (s, 2H), 8.54 (s, 1H), 8.24 (d, J=8.4 Hz, 2H), 8.05 (s, 1H), 7.87 (dd, J=8.5, 1.6 Hz, 1H), 7.79-7.68 (m, 2H), 7.55 (d, J=7.7 Hz, 1H), 7.45 (d, J=8.1 Hz, 1H), 7.40 (d, J=6.8 Hz, 1H), 7.35 (s, 1H), 7.29 (d, J=6.9 Hz, 1H), 7.13 (d, J=12.7 Hz, 2H), 6.40 (s, 1H), 5.61 (s, 1H), 4.82 (s, 1H), 4.68 (d, J=6.8 Hz, 1H), 4.60 (d, J=9.2 Hz, 1H), 4.49 (t, J=8.3 Hz, 1H), 4.44-4.35 (m, 2H), 4.32 (s, 1H), 3.67 (dd, J=10.9, 3.7 Hz, 10H), 3.10 (s, 2H), 2.94-2.75 (m, 3H), 2.73-2.60 (m, 4H), 2.46 (d, J=9.0 Hz, 3H), 2.13-2.04 (m, 1H), 1.96-1.88 (m, 2H), 1.79 (s, 4H), 1.41-1.32 (m, 2H), 1.22 (dd, J=8.3, 2.8 Hz, 2H), 1.08 (d, J=8.3 Hz, 2H), 0.97 (d, J=8.5 Hz, 9H), 0.82 (d, J=4.1 Hz, 2H).

### Example A202. 4-amino-N-((S)-6-(6-(4-((1-(5-fluoro-2-((S)-1-((2S,4R)-1-((S)-2-(1-fluorocyclopropane-1-carboxamido)-3,3-dimethylbutanoyl)-4-hydroxypyrrolidine-2-carboxamido)ethyl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

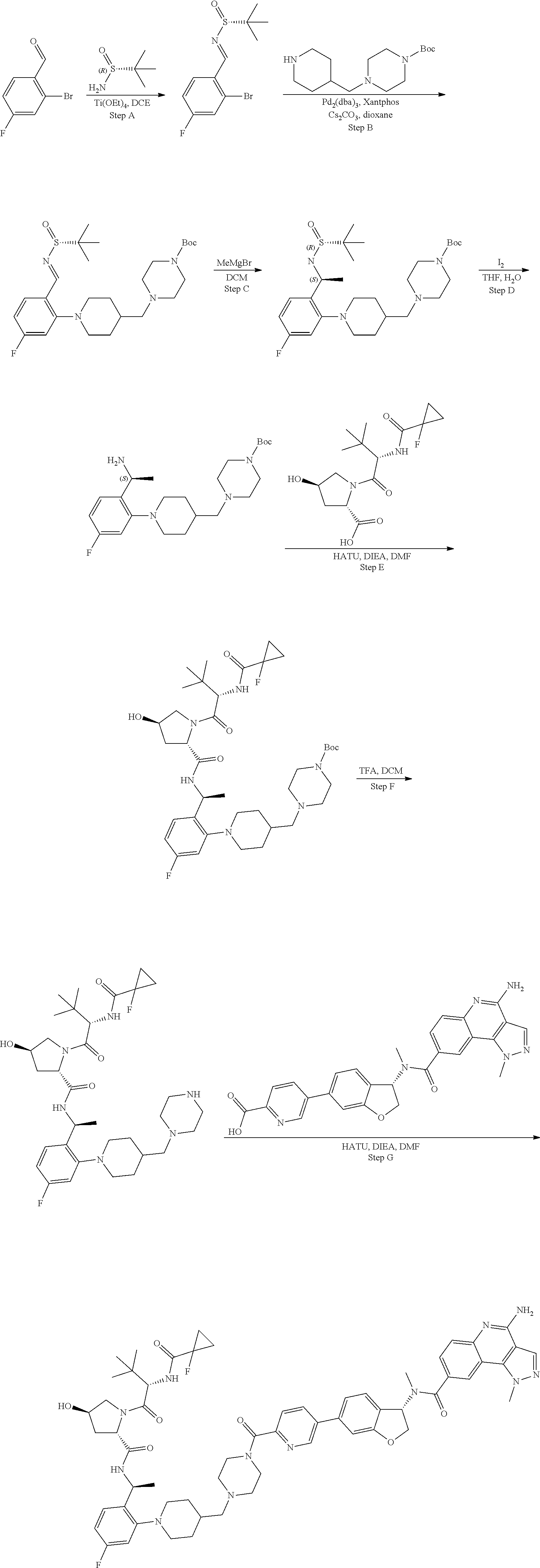

Example A202 was synthesized following the same procedures as described in Example A155 (13.0 mg, 2% yield over 7 steps) as a white solid. LC-MS (ESI): mass calcd. for C₆₀H₇₀F₂N₁₂O₇, 1108.55; m/z found, 1109.7 [M+H]⁺. ¹HNMR (400 MHz, DMSO-d₆) δ 9.71-9.44 (m, 2H), 8.94 (s, 1H), 8.64 (s, 1H), 8.46 (s, 1H), 8.27 (t, J=7.4 Hz, 2H), 7.94 (d, J=8.6 Hz, 1H), 7.88 (s, 1H), 7.78 (d, J=8.1 Hz, 1H), 7.62 (s, 1H), 7.45-7.32 (m, 3H), 7.25 (d, J=6.7 Hz, 1H), 6.95-6.87 (m, 2H), 5.30-5.21 (m, 1H), 4.84-4.70 (m, 2H), 4.59-4.56 (m, 1H), 4.52 (s, 3H), 4.46-4.43 (m, 1H), 4.31-4.22 (m, 3H), 3.73-3.61 (m, 2H), 3.60-3.42 (m, 4H), 3.41-3.22 (m, 3H), 3.21-3.11 (m, 4H), 2.97-2.91 (m, 1H), 2.87-2.79 (m, 1H), 2.74-2.67 (m, 3H), 2.45-2.30 (m, 2H), 2.07-1.85 (m, 3H), 1.80-1.69 (m, 2H), 1.41-1.36 (m, 4H), 1.26 (d, J=6.7 Hz, 3H), 1.23-1.17 (m, 2H), 0.96 (s, 9H).

### Example A203. 2-amino-3-cyclopropyl-N-((S)-6-(6-(4-(2-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)ethyl)piperazine-1-carbonyl)pyridin-3-yl)-2,3-dihydrobenzofuran-3-yl)-N-methylquinoline-6-carboxamide

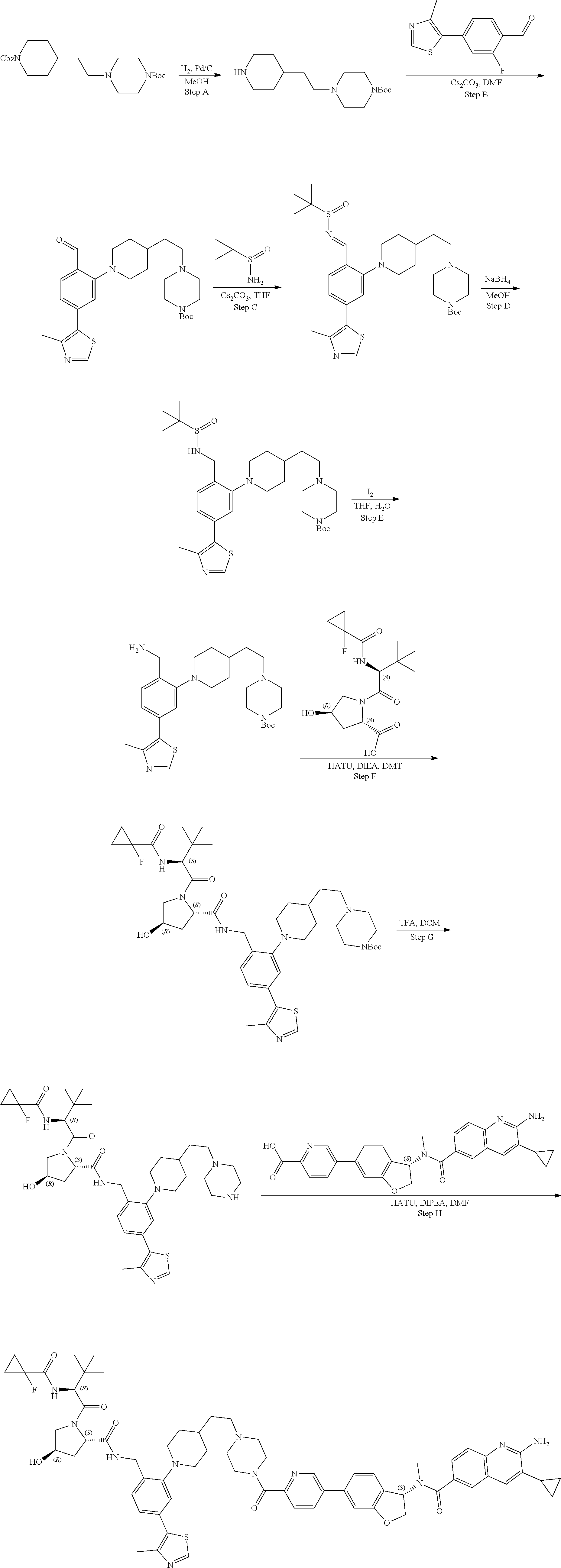

Example A203 was synthesized following the same procedures as described in Example A107 (7 mg, 2% yield over 11 steps) as a white solid. LC-MS (ESI): mass calcd. for C₆₅H₇₆FN₁₁O₇S, 1173.6; m/z found, 1174.2 [M+H]⁺. ¹HNMR (400 MHz, DMSO-d₆) δ 9.84 (s, 1H), 8.99 (s, 1H), 8.93 (s, 1H), 8.85 (s, 1H), 8.56-8.45 (m, 1H), 8.28 (dd, J=8.2, 2.2 Hz, 1H), 8.21 (s, 1H), 8.05 (s, 1H), 7.88-7.85 (m, 1H), 7.78 (d, J=8.2 Hz, 2H), 7.56 (d, J=7.6 Hz, 1H), 7.44-7.33 (m, 3H), 7.29-7.25 (m, 1H), 7.14-7.10 (m, 2H), 4.82-4.67 (m, 2H), 4.60 (d, J=9.1 Hz, 2H), 4.51-4.47 (m, 1H), 4.41-4.31 (m, 4H), 4.28-4.21 (m, 2H), 3.69-3.61 (m, 4H), 3.53-3.47 (m, 2H), 3.26-3.22 (m, 2H), 3.16-3.13 (m, 1H), 3.09-3.05 (m, 2H), 2.76-2.68 (m, 2H), 2.66 (s, 3H), 2.46 (s, 3H), 2.11-2.04 (m, 1H), 1.95-1.88 (m, 2H), 1.81-1.76 (m, 2H), 1.73-1.66 (m, 2H), 1.48-1.44 (m, 1H), 1.42-1.32 (m, 4H), 1.24-1.20 (m, 2H), 1.11-1.06 (m, 2H), 0.98 (s, 9H), 0.83-0.80 (m, 2H).

### Example A204. 2-amino-3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-4-methylpiperidin-4-yl)methyl)piperazine-1-carbonyl)pyridin-3-yl)-2,3-dihydrobenzofuran-3-yl)-N-methylquinoline-6-carboxamide

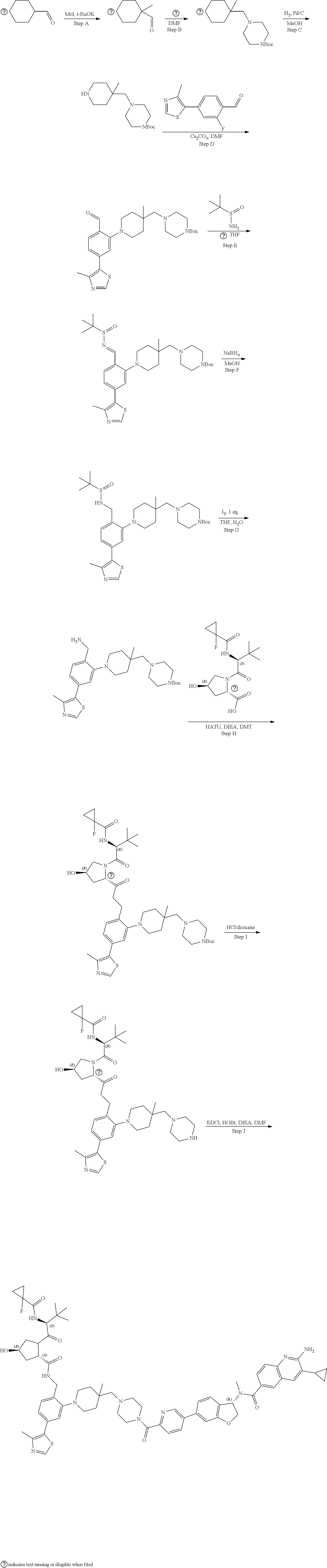

Example A204 was synthesized following the same procedures as described in Example A155 (2.2 mg, 1% yield over 10 steps) as a white solid. LC-MS (ESI): mass calcd. for C₆₅H₇₆FN₁₁O₇S, 1173.6/z found, 1174.6 [M+H]⁺. ¹HNMR (400 MHz, DMSO-d₆) δ 9.17 (s, 1H), 8.99 (s, 1H), 8.93 (s, 1H), 8.79 (s, 2H), 8.52 (s, 1H), 8.28 (d, J=7.8 Hz, 1H), 8.21 (s, 1H), 8.05 (s, 1H), 7.86 (d, J=8.4 Hz, 1H), 7.77 (s, 2H), 7.55 (d, J=7.2 Hz, 1H), 7.43 (d, J=8.2 Hz, 2H), 7.36 (s, 1H), 7.27 (d, J=6.6 Hz, 1H), 7.22 (s, 1H), 7.13 (d, J=8.5 Hz, 1H), 4.75-4.65 (m, 1H), 4.63-4.57 (m, 1H), 4.53-4.44 (m, 1H), 4.43-4.33 (m, 3H), 4.23-4.07 (m, 1H), 3.70-3.60 (m, 8H), 3.30-3.22 (m, 5H), 2.99-2.82 (m, 4H), 2.74-2.62 (m, 3H), 2.46 (s, 3H), 2.12-2.04 (m, 1H), 2.06-1.98 (m, 2H), 1.95-1.87 (m, 2H), 1.78-1.69 (m, 2H), 1.68-1.59 (m, 2H), 1.42-1.32 (m, 2H), 1.22 (s, 3H), 1.08-1.01 (m, 2H), 0.96 (s, 9H), 0.84-0.76 (m, 2H)

### Example A206. 2-amino-3-ethynyl-N-(S)-6-(6-(((15,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-N-methylquinoline-6-carboxamide

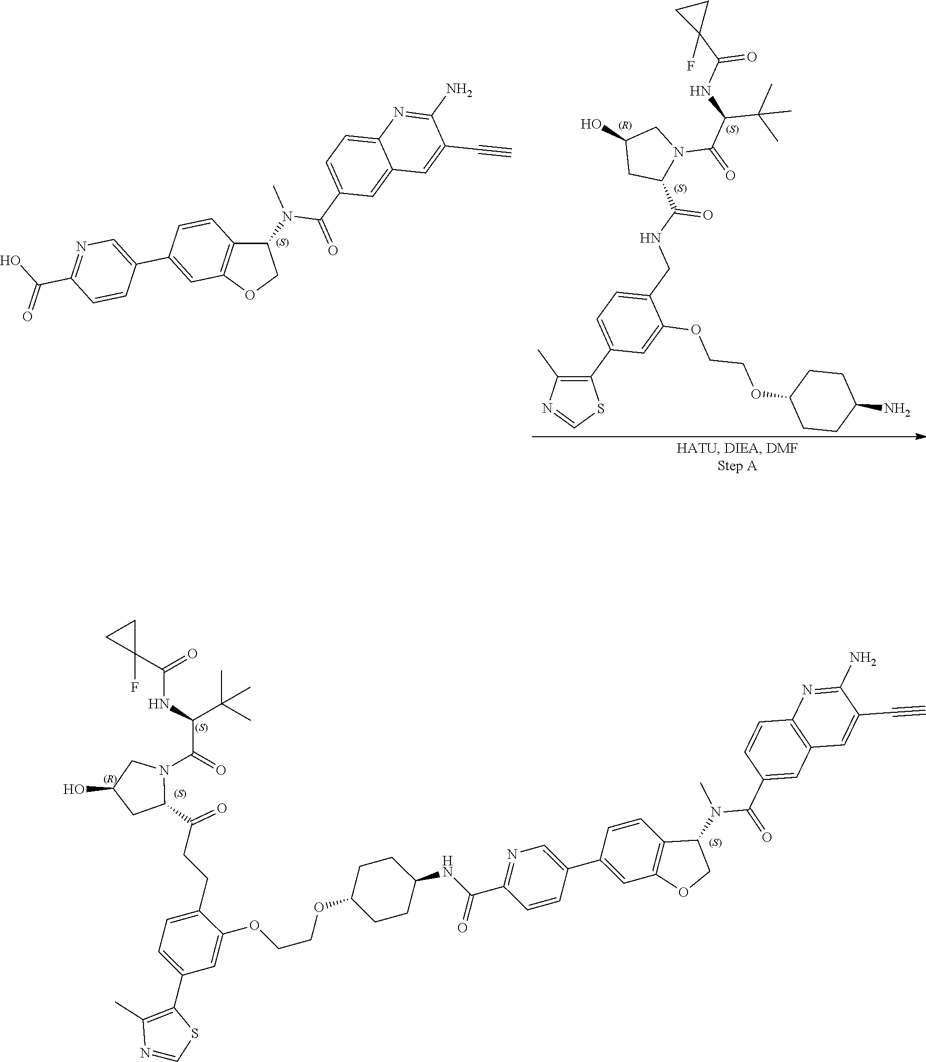

Example A206 was synthesized following the same procedures as described in Example A29 (3.0 mg, 2% yield) as a white solid. LC-MS (ESI): mass calcd, for C₆₁H₆₆FN₉O₉S, 1119.47; m/z found, 1120.9 [M+H]⁺. ¹HNMR (400 MHz, DMSO-d₆) δ 8.99 (s, 1H), 8.92 (s, 1H), 8.63 (s, 1H), 8.49 (t, J=6.8 Hz, 2H), 8.27 (dd, J=8.5, 1.7 Hz, 1H), 8.10-8.00 (m, 2H), 7.87 (d, J=7.7 Hz, 1H), 7.72 (d, J=7.8 Hz, 1H), 7.56 (d, J=7.8 Hz, 1H), 7.46-7.36 (m, 2H), 7.35 (s, 1H), 7.29 (dd, J=8.4, 1.8 Hz, 1H), 7.05 (s, 1H), 6.97 (d, J=7.8 Hz, 1H), 4.91 (s, 1H), 4.71-4.67 (m, 1H), 4.59 (d, J=9.6 Hz, 1H), 4.52 (t, J=8.3 Hz, 1H), 4.37-4.20 (m, 4H), 4.19-4.15 (m, 2H), 3.65-3.58 (m, 5H), 3.41-3.32 (m, 2H), 2.66 (s, 3H), 2.47 (s, 3H), 2.15-2.01 (m, 3H), 1.97-1.79 (m, 3H), 1.58-1.45 (m, 2H), 1.40-1.27 (m, 4H), 1.23-1.19 (m, 2H), 0.95 (s, 9H).

### Example A207. 2-amino-3-cyano-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-N-methylquinoline-6-carboxamide

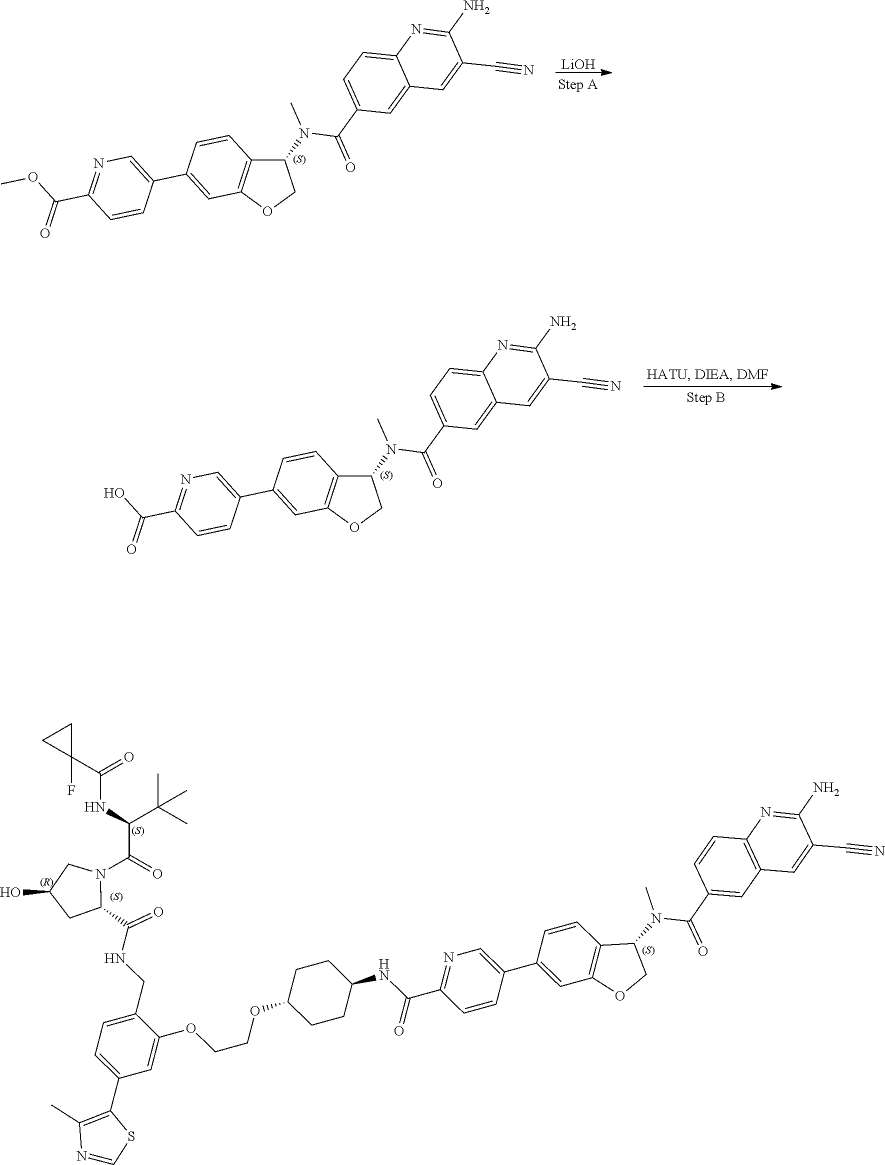

Example A207 was synthesized following the same procedures as described in Example A29 (11.0 mg, 5% yield over 2 steps) as a yellow solid. LC-MS (ESI): mass calcd. for C₆₀H₆₅FN₁₀O₉S, 1120.46; m/z found, 1121.8 [M+H]⁺. ¹HNMR (400 MHz, DMSO-d₆) δ 9.00 (s, 1H), 8.92 (d, J=1.8 Hz, 1H), 8.84 (s, 1H), 8.52-8.45 (m, 2H), 8.27 (dd, J=8.1, 2.1 Hz, 1H), 8.08 (d, J=8.2 Hz, 1H), 7.98 (s, 1H), 7.83 (dd, J=8.7, 1.8 Hz, 1H), 7.68-7.45 (m, 4H), 7.42-7.37 (m, 2H), 7.34 (s, 1H), 7.29 (dd, J=9.2, 2.5 Hz, 1H), 7.05 (d, J=1.2 Hz, 1H), 6.97 (d, J=7.8 Hz, 1H), 4.72-4.66 (m, 2H), 4.60 (d, J=9.2 Hz, 2H), 4.54-4.50 (m, 2H), 4.25 (d, J=5.5 Hz, 2H), 4.19-4.16 (m, 2H), 3.84-3.78 (m, 3H), 3.68-3.59 (m, 2H), 3.39-3.33 (m, 1H), 2.67 (s, 3H), 2.47 (s, 3H), 2.12-1.84 (m, 6H), 1.56-1.46 (m, 2H), 1.40-1.28 (m, 4H), 1.24-1.19 (m, 2H), 0.95 (s, 9H).

### Example A208. 2-amino-N-((3S)-6-(6-(5-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl) octahydropyrrolo[3,4-c]pyrrole-2-carbonyl)pyridin-3-yl)-2,3-dihydrobenzofuran-3-yl)-N-methyl-3-(oxetan-3-yl)quinoline-6-carboxamide

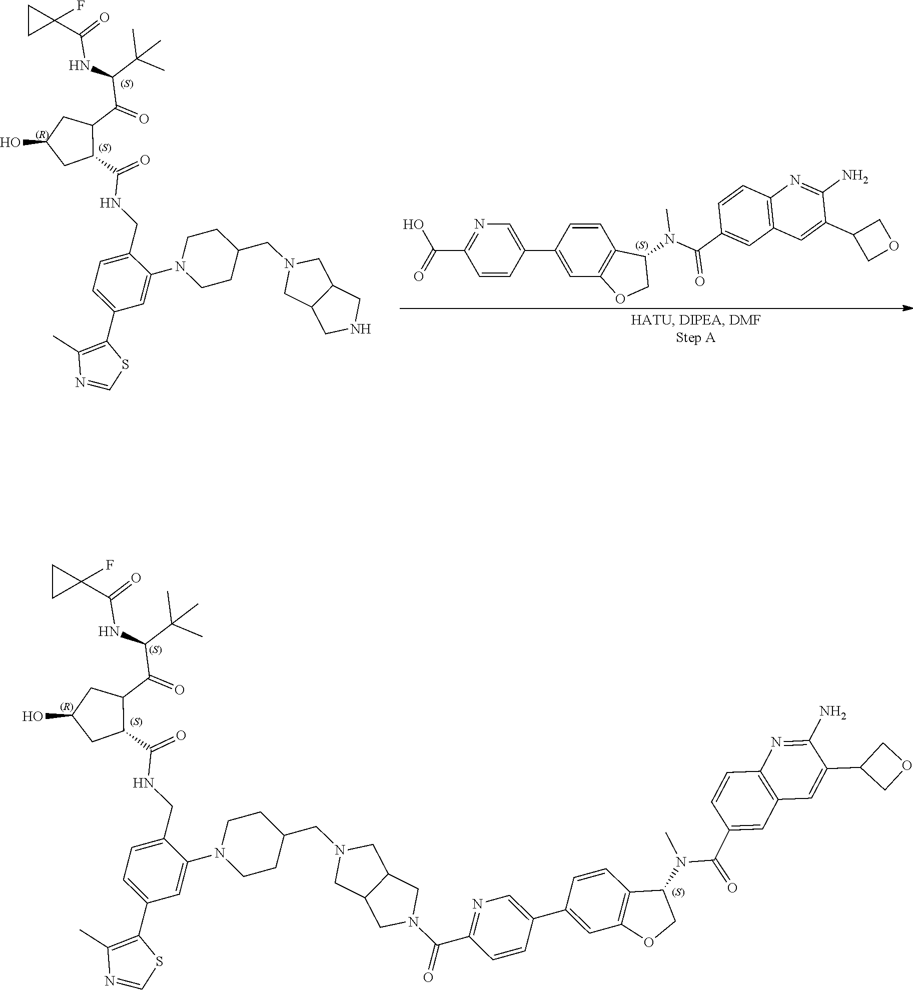

Example A208 was synthesized following the same procedures as described in Example A192 (32 mg, 8% yield over 5 steps) as a white solid. LC-MS (ESI): mass calcd. For C₆₆H₇₆FN₁₁O₈S, 1201.56/z found, 1203 [M+H]⁺. ¹H NMR (400 MHz, DMSO-d₆) δ 9.70 (s, 1H), 8.99 (s, 1H), 8.93 (s, 1H), 8.69 (s, 2H), 8.52 (s, 1H), 8.25 (d, J=8.2 Hz, 1H), 8.18 (s, 1H), 7.92 (d, J=8.5 Hz, 1H), 7.86 (t, J=8.1 Hz, 1H), 7.78 (s, 1H), 7.57 (d, J=7.7 Hz, 1H), 7.44 (d, J=8.4 Hz, 1H), 7.40 (d, J=7.4 Hz, 1H), 7.35 (s, 1H), 7.27 (d, J=7.1 Hz, 1H), 7.13 (d, J=6.3 Hz, 2H), 5.06-4.95 (m, 1H), 4.83 (s, 1H), 4.78-4.73 (m, 2H), 4.72-4.66 (m, 1H), 4.60 (d, J=9.1 Hz, 1H), 4.49 (t, J=8.2 Hz, 1H), 4.44-4.40 (m, 1H), 4.38-4.34 (m, 2H), 4.33-4.27 (m, 1H), 4.09-3.95 (m, 1H), 3.95-3.78 (m, 4H), 3.73-3.56 (m, 4H), 3.46-3.33 (m, 1H), 3.28-3.16 (m, 3H), 3.12-2.99 (m, 4H), 2.99-2.90 (m, 1H), 2.74 (d, J=10.9 Hz, 2H), 2.68 (s, 3H), 2.46 (s, 3H), 2.13-2.05 (m, 1H), 1.95-1.80 (m, 4H), 1.43 (s, 1H), 1.43-1.36 (m, 2H), 1.36-1.31 (m, 1H), 1.22 (d, J=8.1 Hz, 2H), 0.97 (s, 9H).).

### Example A209. 2-amino-3-cyclopropyl-7-fluoro-N-((S)-6-(6-(4-((1-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

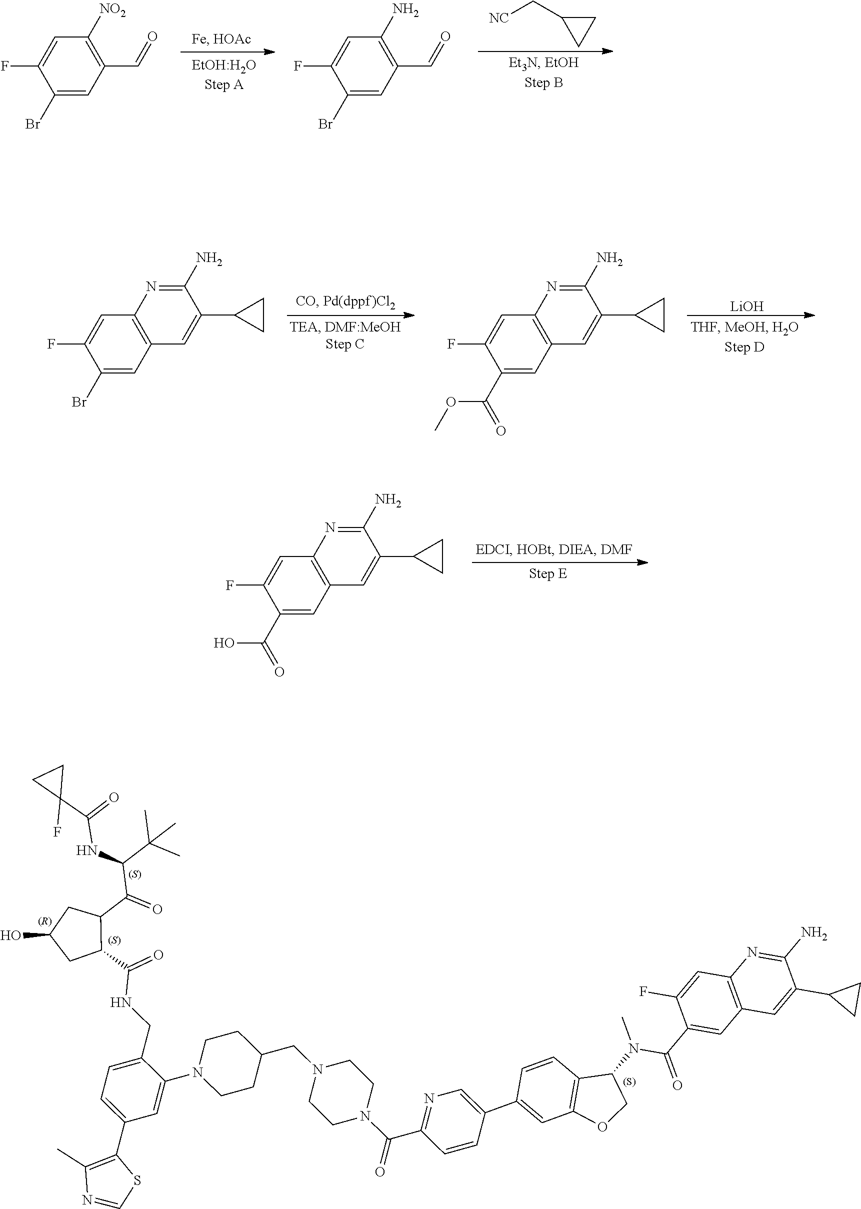

Example A209 was synthesized following the same procedures as described in Example A199 (4.6 mg, 2% yield over 5 steps) as a white solid. LC-MS (ESI): mass calcd. For C₆₄H73F₂N11O7S, 1177.54/z found, 1178.5 [M+H]⁺. ¹HNMR (400 MHz, DMSO-d₆) δ 9.62 (s, 1H), 8.99 (s, 1H), 8.93 (d, J=5.7 Hz, 1H), 8,90-8.82 (m, 1H), 8.51 (t, J=5.7 Hz, 1H), 8.28 (t, J=6.7 Hz, 1H), 8.15 (d, J=13.2 Hz, 1H), 8.03 (d, J=7.2 Hz, 1H), 7.78 (dd, J=8.0, 4.4 Hz, 1H), 7.58-7.53 (m, 1H), 7.50-7.45 (m, 1H), 7.44-7.39 (m, 2H), 7.36 (d, J=16.1 Hz, 1H), 7.27 (dd, J=9.1, 2.4 Hz, 1H), 7.13 (d, J=4.4 Hz, 2H), 6.48-6.44 (m, 1H), 5.49 (s, 1H), 4.87-4.80 (m, 1H), 4.66 (d, J=10.6 Hz, 2H), 4.60 (d, J=9.2 Hz, 2H), 4.49 (s, 1H), 4.35 (s, 2H), 4.22 (s, 1H), 3.64 (dd, J=21.6, 9.0 Hz, 4H), 3.58-3.48 (m, 2H), 3.33 (s, 1H), 3.17 (s, 4H), 3.11 (d, J=10.8 Hz, 3H), 2.72 (s, 3H), 2.56 (s, 1H), 2.46 (s, 3H), 2.12-2.06 (m, 1H), 1.98 (s, 1H), 1.90 (d, J=12.2 Hz, 4H), 1.43 (s, 2H), 1.37 (dd, J=15.7, 7.1 Hz, 2H), 1.25-1.19 (m, 2H), 1.07 (s, 2H), 0.96 (d, J=8.6 Hz, 9H), 0.78 (s, 2H).

### Example A210. 2-amino-3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl) azetidin-3-yl)methyl)piperazine-1-carbonyl)pyridin-3-yl)-2,3-dihydrobenzofuran-3-yl)-N-methylquinoline-6-carboxamide

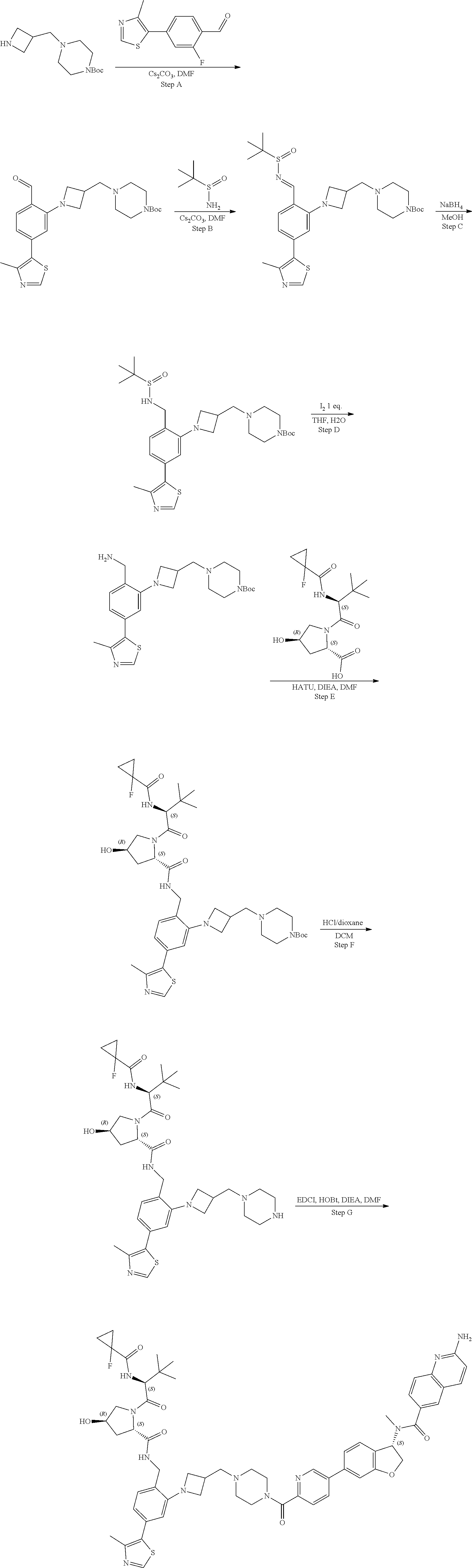

Example A210 was synthesized following the same procedures as described in Example A107 (4.6 mg, 8% yield over 6 steps) as a white solid. LC-MS (ESI): mass calcd. For C₆₂H7FN₁₁O₇S, 1131.52/z found, 1132.3 [M+H]⁺. ¹HNMR (400 MHz, DMSO-d₆) δ 8.96 (s, 1H), 8.93 (s, 1H), 8.82 (s, 2H), 8.58 (s, 1H), 8.27 (d, J=8.4 Hz, 1H), 8.21 (s, 1H), 8.04 (s, 1H), 7.86 (d, J=8.7 Hz, 1H), 7.75 (d, J=8.0 Hz, 2H), 7.55 (d, J=7.2 Hz, 1H), 7.40 (d, J=6.1 Hz, 1H), 7.35 (s, 1H), 7.27 (d, J=6.9 Hz, 1H), 7.21 (d, J=7.8 Hz, 1H), 6.69 (d, J=7.7 Hz, 1H), 6.64 (s, 1H), 5.47 (d, J=7.1 Hz, 1H), 4.69 (s, 1H), 4.59 (d, J=8.9 Hz, 1H), 4.48 (t, J=8.0 Hz, 1H), 4.40 (s, 1H), 4.36 (s, 2H), 4.29-4.21 (m, 2H), 4.16 (d, J=4.9 Hz, 2H), 3.97-3.81 (m, 6H), 3.73-3.61 (m, 6H), 2.65 (s, 5H), 2.46 (s, 3H), 2.11-2.04 (m, 1H), 1.93-1.86 (m, 2H), 1.38-1.29 (m, 3H), 1.23-1.17 (m, 2H), 1.08 (d, J=7.8 Hz, 2H), 0.95 (s, 9H), 0.81 (d, J=2.5 Hz, 2H).).

### Example A211. 2-amino-3-cyano-N-((S)-6-(6-((4-(2-(2-(((2S,4S)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)butyl)carbamoyl)pyridin-3-yl)-2,3-dihydrobenzofuran-3-yl)-N-methylquinoline-6-carboxamide

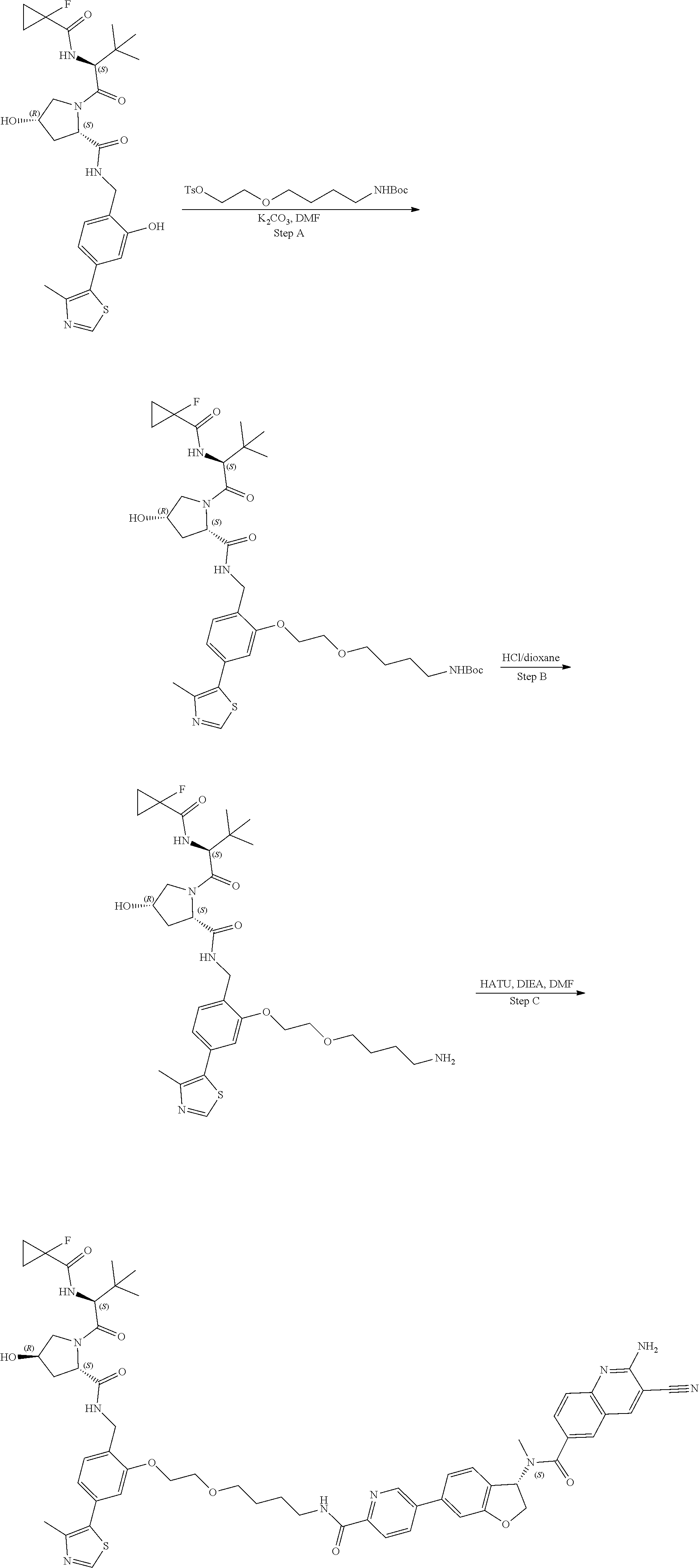

Example A211 was synthesized following the same procedures as described in Example A23 (11.8 mg, 17% yield over 3 steps) as a white solid. LC-MS (ESI): mass calcd. for C₅₈H₆₃FN₁₀O₇S, 1094.4; m/z found, 1095.2 [M+H]⁺. ¹H NMR (400 MHz, DMSO-d₆) δ 8.97 (s, 1H), 8.91 (s, 1H), 8.85-8.78 (m, 2H), 8.55 (t, J=5.7 Hz, 1H), 8.29-8.24 (m, 1H), 8.07 (d, J=8.1 Hz, 1H), 7.97 (s, 1H), 7.82 (d, J=8.7 Hz, 1H), 7.60 (d, J=7.6 Hz, 1H), 7.55 (d, J=7.7 Hz, 1H), 7.45 (s, 1H), 7.39 (d, J=7.7 Hz, 2H), 7.36-7.27 (m, 3H), 7.04 (s, 1H), 6.94 (d, J=7.9 Hz, 1H), 4.70-4.68 (m, 1H), 4.54 (d, J=8.8 Hz, 1H), 4.44 (s, 1H), 4.33 (d, J=10.1 Hz, 1H), 4.23 (d, J=2.9 Hz, 1H), 4.20 (s, 1H), 4.19 (s, 2H), 3.85 (dd, J=9.9, 5.7 Hz, 1H), 3.77-3.74 (m, 2H), 3.52 (t, J=5.7 Hz, 2H), 3.48-3.44 (m, 1H), 3.33 (d, J=6.1 Hz, 2H), 2.67 (s, 3H), 2.44 (s, 3H), 2.39-2.31 (m, 3H), 1.75 (dt, J=12.0, 5.8 Hz, 1H), 1.58 (s, 4H), 1.34 (d, J=14.9 Hz, 2H), 1.20 (d, J=8.6 Hz, 2H), 0.96 (s, 9H).

### Example A213. 2-amino-3-cyano-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

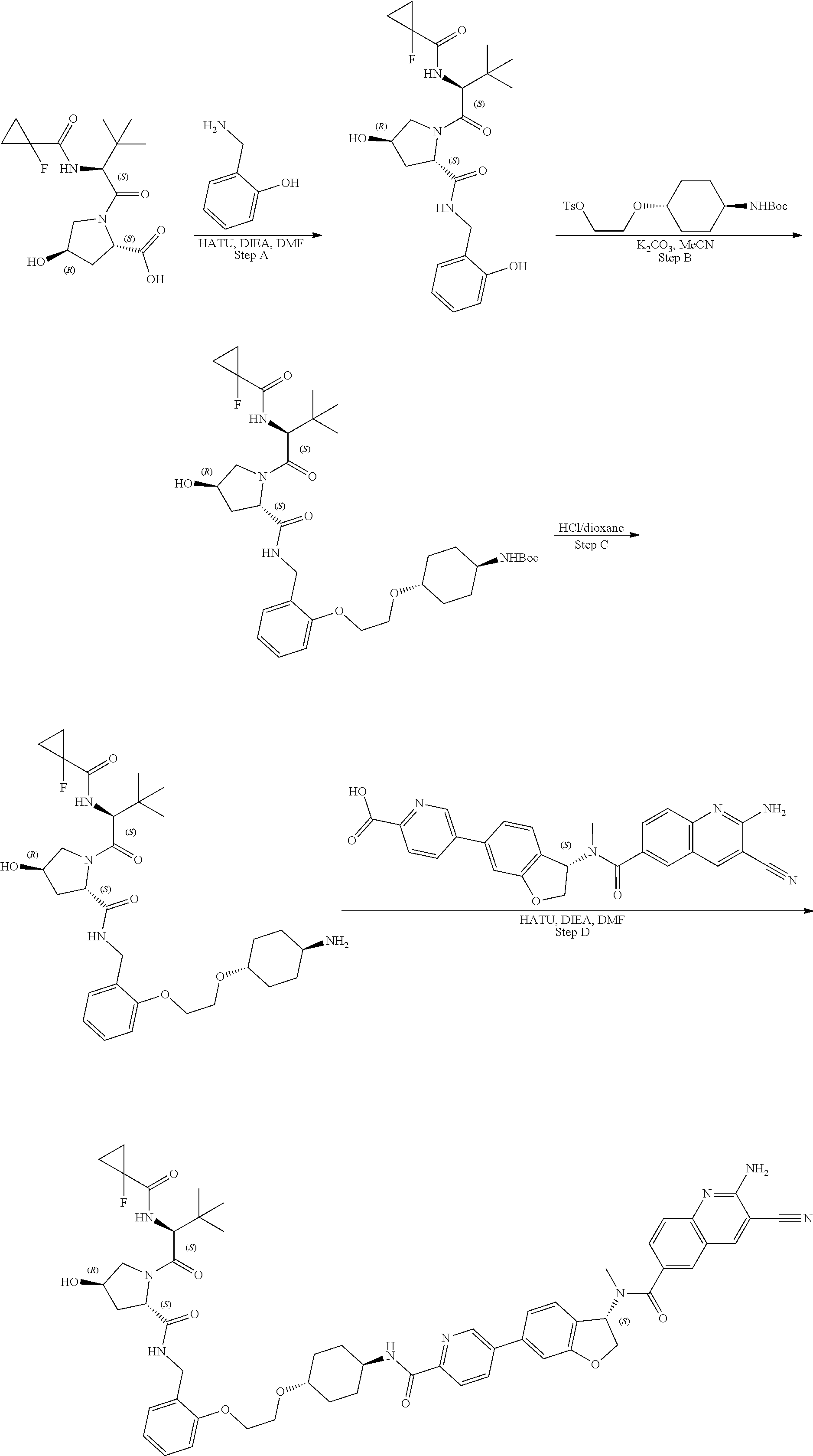

Example A213 was synthesized following the same procedures as described in Example A29 (7.5 mg, 4% yield over 4 steps) as a yellow solid. LC-MS (ESI): mass calcd. for C₅₆H₆₂FN₉O₉, 1023.46; m/z found, 1024.7 [M+H]⁺. ¹HNMR (400 MHz, DMSO-d₆) δ 8.92 (d, J=1.8 Hz, 1H), 8.83 (s, 1H), 8.48 (d, J=8.5 Hz, 1H), 8.39 (t, J=5.9 Hz, 1H), 8.27 (dd, J=8.2, 2.1 Hz, 1H), 8.08 (d, J=8.1 Hz, 1H), 7.98 (s, 1H), 7.82 (dd, J=8.7, 1.8 Hz, 1H), 7.60 (d, J=8.2 Hz, 1H), 7.56 (d, J=7.8 Hz, 1H), 7.48 (s, 1H), 7.39 (d, J=7.6 Hz, 1H), 7.34 (s, 1H), 7.32-7.25 (m, 2H), 7.21-7.17 (m, 1H), 6.96 (d, J=8.1 Hz, 1H), 6.86 (t, J=7.4 Hz, 1H), 4.69 (dd, J=9.9, 3.9 Hz, 2H), 4.59 (d, J=9.3 Hz, 2H), 4.53-4.48 (m, 3H), 4.35 (s, 1H), 4.28-4.22 (m, 2H), 4.10-4.08 (m, 2H), 3.80-3.78 (m, 2H), 3.67-3.57 (m, 2H), 3.39-3.32 (m, 1H), 2.67 (s, 3H), 2.11-2.01 (m, 3H), 1.95-1.83 (m, 3H), 1.53-1.47 (m, 2H), 1.40-1.28 (m, 4H), 1.21 (dd, J=8.3, 2.9 Hz, 2H), 0.95 (s, 9H).

### Example A214. 2-amino-3-cyano-N-((3S)-6-(6-(5-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl) octahydropyrrolo[3,4-c]pyrrole-2-carbonyl)pyridin-3-yl)-2,3-dihydrobenzofuran-3-yl)-N-methylquinoline-6-carboxamide

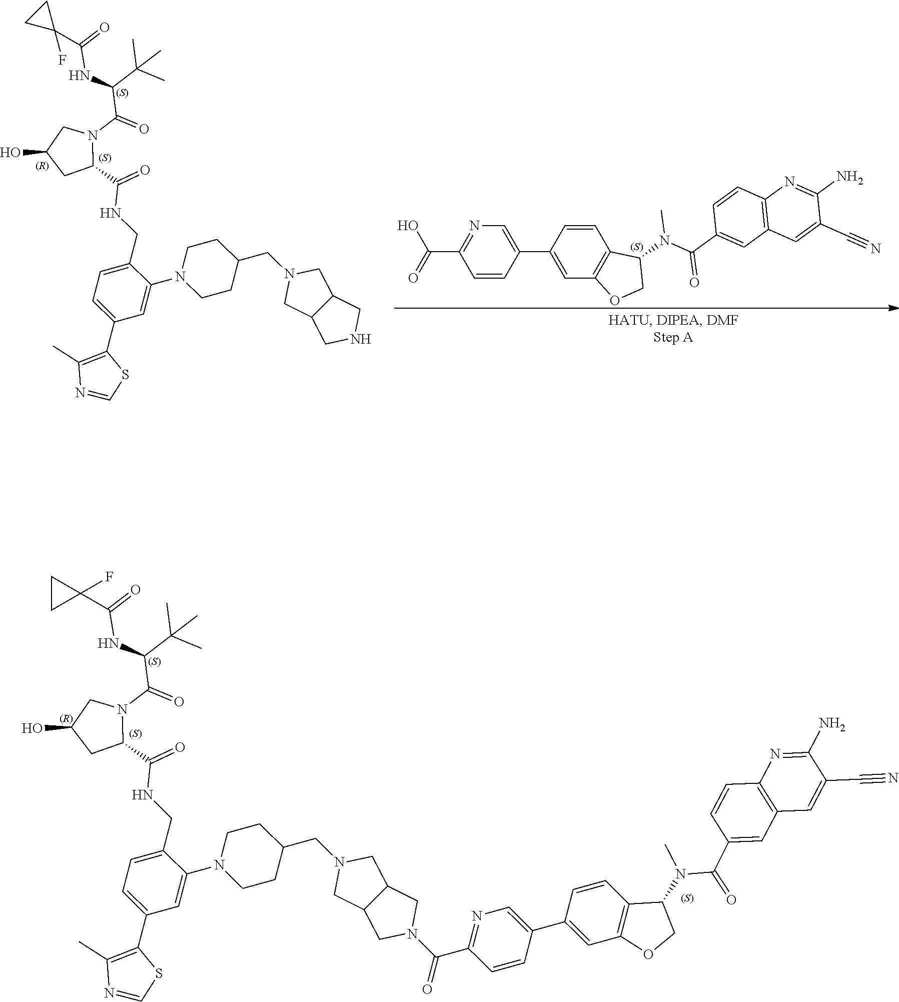

Example A214 was synthesized following the same procedures as described in Example A192 (5.6 mg, 4% yield over 4 steps) as a white solid. LC-MS (ESI): mass calcd. for C₆₄H₇₁FN₁₂O₇S, 1170.53; m/z found, 1172.0 [M+H]⁺. ¹HNMR (400 MHz, DMSO-d₆) δ 9.56 (s, 1H), 8.99 (s, 1H), 8.92 (s, 1H), 8.80 (s, 1H), 8.51 (t, J=5.5 Hz, 1H), 8.25 (dd, J=8.1, 1.8 Hz, 1H), 7.97 (s, 1H), 7.85 (t, J=7.4 Hz, 1H), 7.81 (dd, J=8.7, 1.5 Hz, 1H), 7.60-7.54 (m, 2H), 7.43 (d, J=8.3 Hz, 1H), 7.39 (d, J=7.7 Hz, 2H), 7.34 (s, 1H), 7.27 (dd, J=9.6, 2.3 Hz, 1H), 7.13 (d, J=5.7 Hz, 2H), 4.69 (dd, J=9.2, 3.9 Hz, 1H), 4.60 (d, J=9.1 Hz, 1H), 4.49 (t, J=7.9 Hz, 1H), 4.39-4.31 (m, 3H), 3.91-3.80 (m, 6H), 3.26-3.16 (m, 4H), 3.13-2.99 (m, 6H), 2.73-2.65 (m, 6H), 2.46 (s, 3H), 2.11-2.06 (m, 1H), 1.95-1.79 (m, 5H), 1.48-1.32 (m, 4H), 1.22 (dd, J=8.7, 1.7 Hz, 2H), 0.97 (s, 9H).

### Example A215. 2-amino-3-cyano-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)phenyl)carbamoyl)pyridin-3-yl)-2,3-dihydrobenzofuran-3-yl)-N-methylquinoline-6-carboxamide

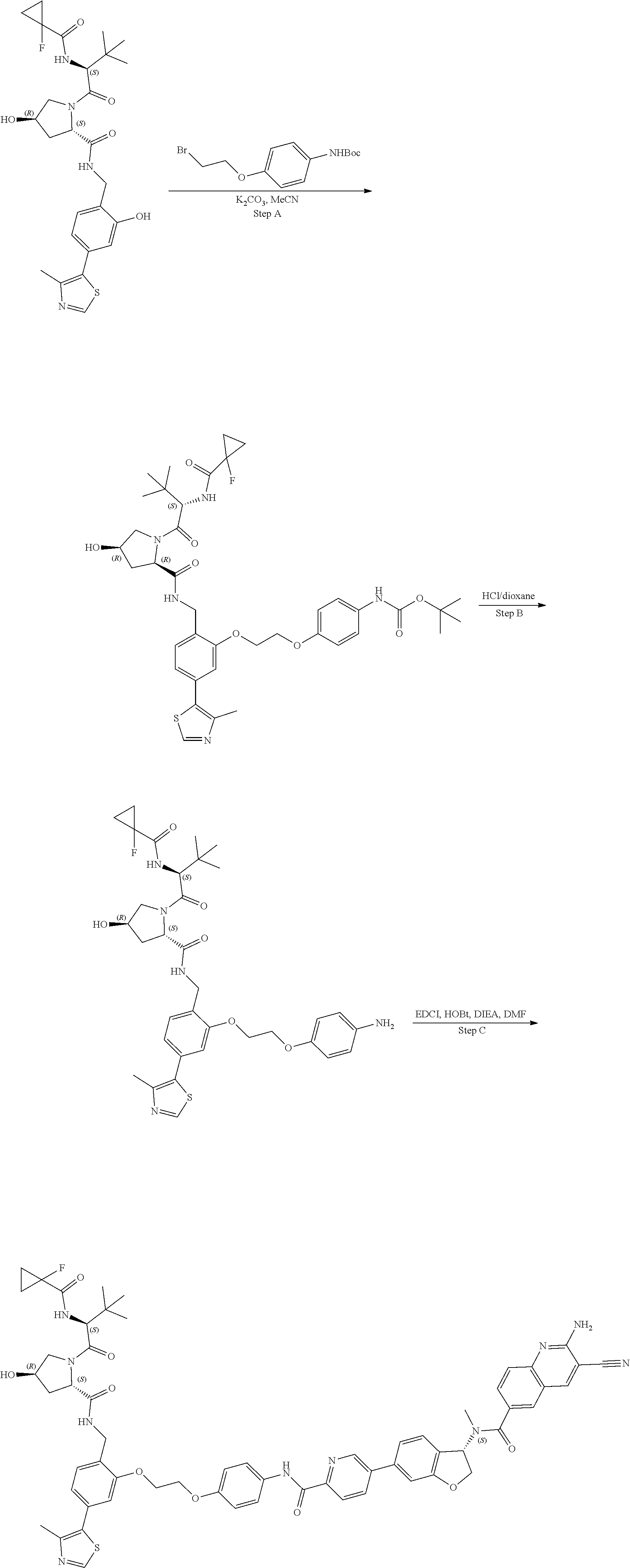

Example A215 was synthesized following the same procedures as described in Example A23 (6.4 mg, 6% yield over 3 steps) as a yellow solid. LC-MS (ESI): mass calcd. for C₆₀H₅₉FN₁₀O₉S, 1114.42; m/z found, 1115.2 [M+H]⁺. ¹H NMR (400 MHz, DMSO-d₆) δ 10.59 (s, 1H), 9.05-8.97 (m, 2H), 8.82 (s, 1H), 8.50 (t, J=5.7 Hz, 1H), 8.37-8.31 (m, 1H), 8.20 (d, J=8.2 Hz, 1H), 7.98 (s, 1H), 7.87-7.80 (m, 3H), 7.62-7.55 (m, 2H), 7.45-7.41 (m, 2H), 7.39 (s, 1H), 7.32-7.27 (m, 1H), 7.11 (s, 1H), 7.03 (s, 1H), 7.01-6.97 (m, 2H), 4.72-4.68 (m, 2H), 4.61-4.58 (m, 1H), 4.54-4.48 (m, 2H), 4.43-4.41 (m, 2H), 4.37-4.34 (m, 2H), 4.29 (d, J=5.8 Hz, 1H), 4.24-4.17 (m, 2H), 3.67-3.58 (m, 2H), 2.68 (s, 3H), 2.47 (s, 3H), 2.11-2.04 (m, 1H), 1.95-1.87 (m, 1H), 1.41-1.31 (m, 2H), 1.24-1.19 (m, 2H), 0.96 (s, 9H).

### Example A216. 2-amino-3-cyano-N-((S)-6-(6-((6-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) spiro[3.3]heptan-2-yl)carbamoyl)pyridin-3-yl)-2,3-dihydrobenzofuran-3-yl)-N-methylquinoline-6-carboxamide

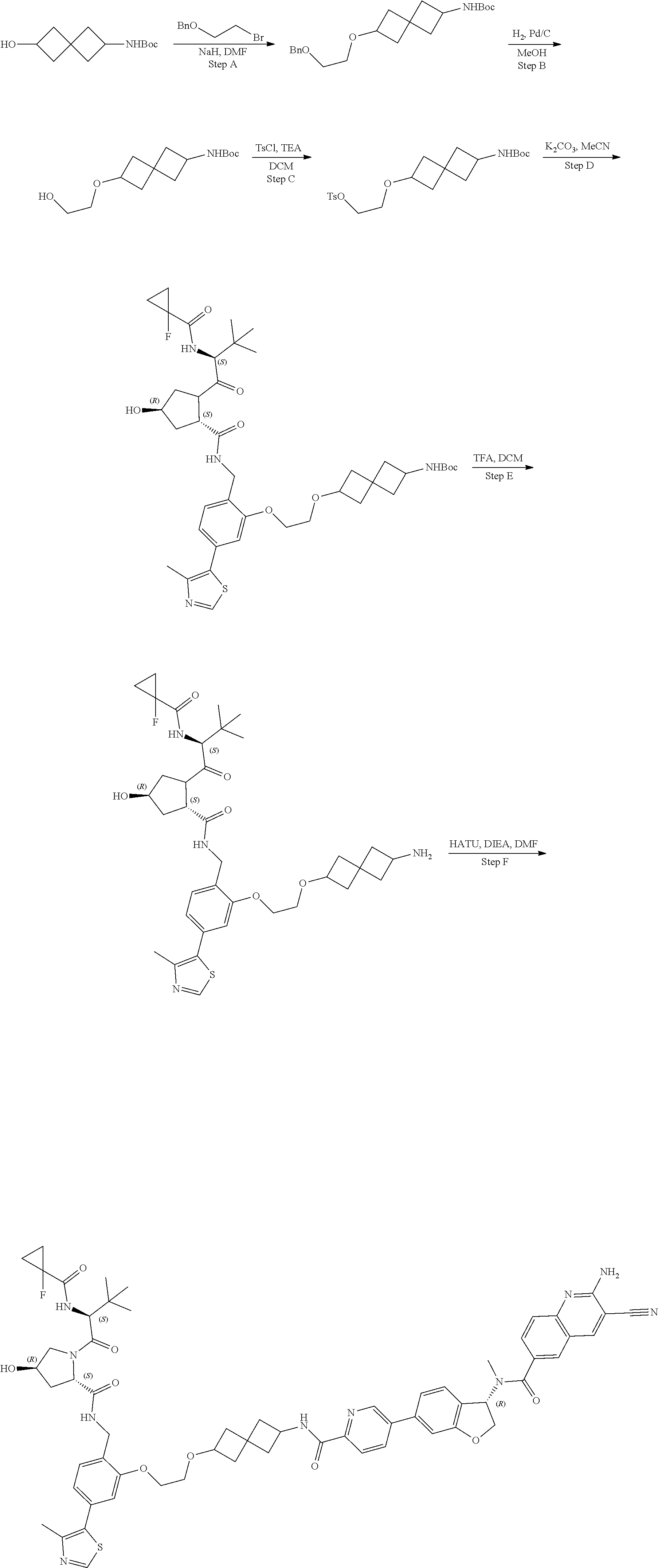

Example A216 was synthesized following the same procedures as described in Example A29 (3.0 mg, 6% yield over 6 steps) as a white solid. LC-MS (ESI): mass calcd. for C₆₁H₆₈FN₁₀O₉S, 1132.46/z found, 1133.8 [M+H]⁺, ¹H NMR (400 MHz, DMSO-d₆) δ 9.00 (s, 1H), 8.93-8.82 (m, 3H), 8.53-8.43 (m, 1H), 8.29-8.24 (m, 1H), 8.06 (d, J=8.2 Hz, 1H), 7.98 (s, 1H), 7.85-7.81 (m, 1H), 7.64-7.52 (m, 3H), 7.42-7.38 (m, 2H), 7.35 (s, 1H), 7.31-7.26 (m, 1H), 7.05-7.03 (m, 1H), 6.99-6.96 (m, 1H), 4.83-4.67 (m, 2H), 4.59 (d, J=9.2 Hz, 1H), 4.54-4.49 (m, 1H), 4.41-4.33 (m, 2H), 4.30-4.23 (m, 2H), 4.21-4.13 (m, 3H), 3.67-3.60 (m, 5H), 2.67 (s, 3H), 2.47 (s, 3H), 2.35-2.18 (m, 6H), 2.12-2.06 (m, 1H), 1.97-1.87 (m, 3H), 1.40-1.31 (m, 2H), 1.24-1.19 (m, 2H), 0.95 (s, 9H).

### Example A217. N-((S)-6-(6-(((1S,4S)-4-(2-(2-(((2S,4R)-1-((S)-2-acetamido-3,3-dimethylbutanoyl)-4-hydroxypyrrolidine-2-carboxamido)methyl)-5-fluorophenoxy)ethoxy) cyclohexyl)carbamoyl)pyridin-3-yl)-2,3-dihydrobenzofuran-3-yl)-2-amino-3-cyano-N-methylquinoline-6-carboxamide

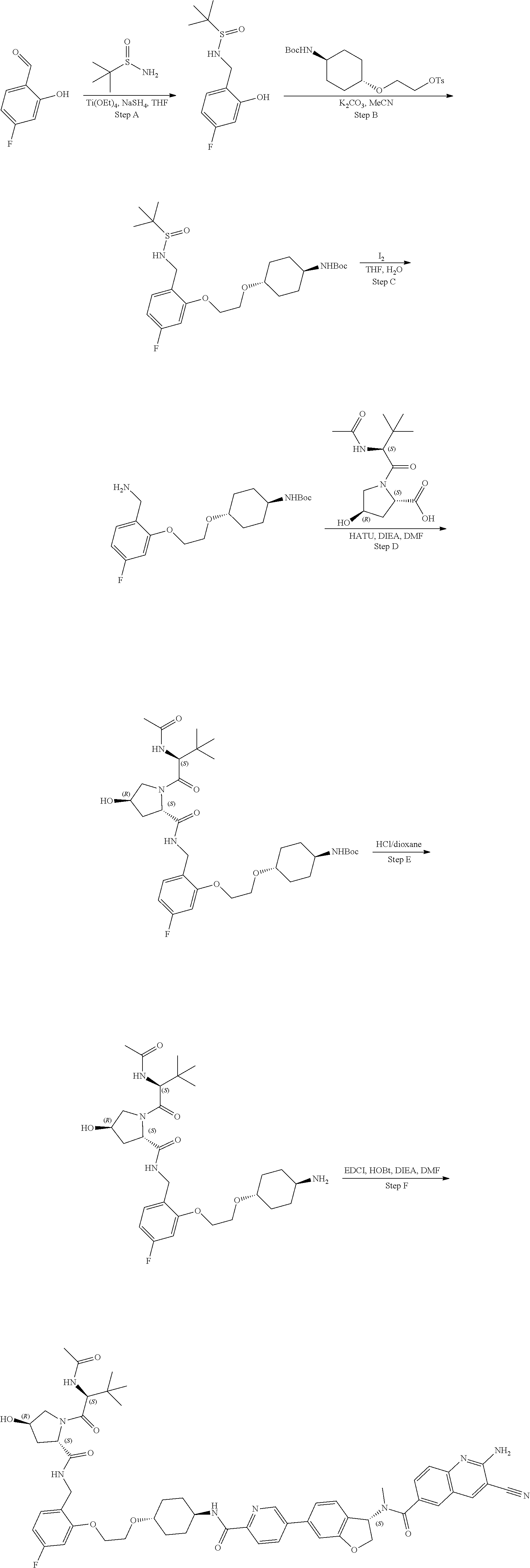

Example A217 was synthesized following the same procedures as described in Example A126 (3.8 mg, 7% yield over 2 steps) as a white solid. LC-MS (ESI): mass calcd. for C₅₄H₆₀FN₉O₉, 997.45; m/z found, 998.5 [M+H]⁺. ¹H NMR (400 MHz, DMSO-d₆) δ 8.93 (s, 1H), 8.80 (s, 1H), 8.50 (d, J=8.5 Hz, 1H), 8.37 (t, J=6.0 Hz, 1H), 8.28 (dd, J=8.2, 2.0 Hz, 1H), 8.08 (d, J=8.2 Hz, 1H), 7.95 (d, J=10.7 Hz, 2H), 7.80 (dd, J=8.8, 1.7 Hz, 1H), 7.62-7.54 (m, 2H), 7.37 (dd, J=15.7, 7.5 Hz, 5H), 6.89 (dd, J=11.3, 2.3 Hz, 1H), 6.60 (td, J=8.5, 2.3 Hz, 1H), 5.32 (t, J=5.0 Hz, 1H), 4.77 (s, 1H), 4.71-4.67 (m, 1H), 4.53 (d, J=9.4 Hz, 1H), 4.43 (t, J=8.0 Hz, 1H), 4.34 (s, 1H), 4.26 (dd, J=16.4, 6.4 Hz, 1H), 4.10 (dd, J=9.0, 5.7 Hz, 3H), 3.79 (d, J=3.9 Hz, 4H), 3.65 (dd, J=14.1, 9.8 Hz, 3H), 2.67 (s, 3H), 2.05 . . . 2.00 (m, 3H), 1.89 (s, 3H), 1.87-1.81 (m, 1H), 1.59-1.41 (m, 3H), 1.35-1.26 (m, 4H), 0.92 (s, 9H).

### Example A218. 2-amino-3-cyano-N-((S)-6-(6-(4-((1-(2-(((2S,4S)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

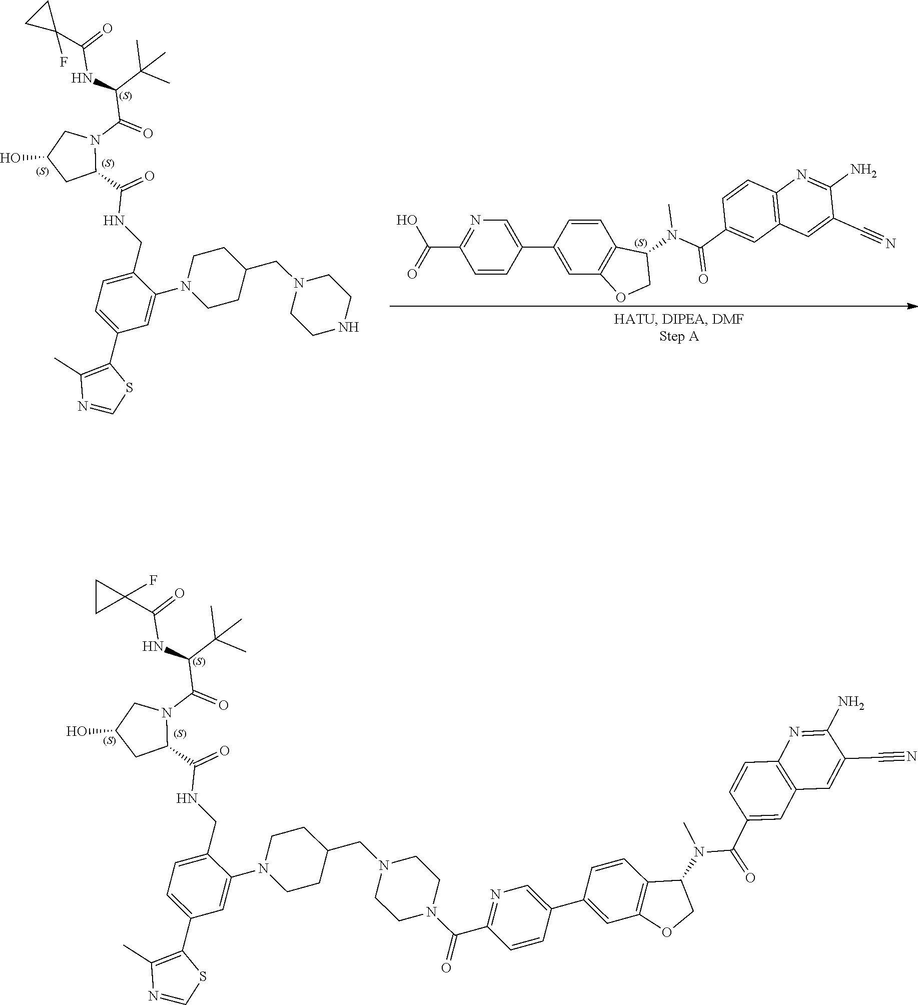

Example A218 was synthesized following the same procedures as described in Example A107 (7.1 mg, 2% yield over 7 steps) as a white solid. LC-MS (ESI): mass calcd. for C₆₂H₆₉FN₁₂O₇S, 1144.51; m/z found, 1046.0 [M+H]⁺. ¹H NMR (400 MHz, DMSO-d₆) δ 9.58 (s, 1H), 9.00 (s, 1H), 8.94 (s, 1H), 8.83 (s, 1H), 8.59 (s, 1H), 8.29 (dd, J=8.2, 2.2 Hz, 1H), 7.98 (s, 1H), 7.80 (dd, J=15.0, 8.3 Hz, 2H), 7.62-7.55 (m, 2H), 7.42 (dd, J=16.2, 7.9 Hz, 3H), 7.35 (s, 1H), 7.30 (d, J=6.6 Hz, 1H), 7.14 (d, J=10.3 Hz, 2H), 4.71-4.68 (m, 1H), 4.64-4.60 (m, 1H), 4.57-4.54 (m, 1H), 4.43-4.36 (m, 3H), 4.27-4.23 (m, 2H), 3.89-3.86 (m, 2H), 3.70-3.66 (m, 1H), 3.63-3.53 (m, 2H), 3.48-3.44 (m, 1H), 3.36-3.28 (m, 1H), 3.21-3.15 (m, 3H), 3.14-3.09 (m, 2H), 2.78-2.69 (m, 2H), 2.68 (s, 3H), 2.46 (s, 3H), 2.38-2.32 (m, 1H), 2.05-1.95 (m, 1H), 1.92-1.83 (m, 2H), 1.79-1.71 (m, 1H), 1.51-1.40 (m, 2H), 1.39-1.28 (m, 2H), 1.23-1.15 (m, 2H), 0.99 (s, 9H).

### Example A219. 2-amino-3-cyano-N-((S)-6-(6-(((1S,4S)-4-(2-(5-fluoro-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-N-methylquinoline-6-carboxamide

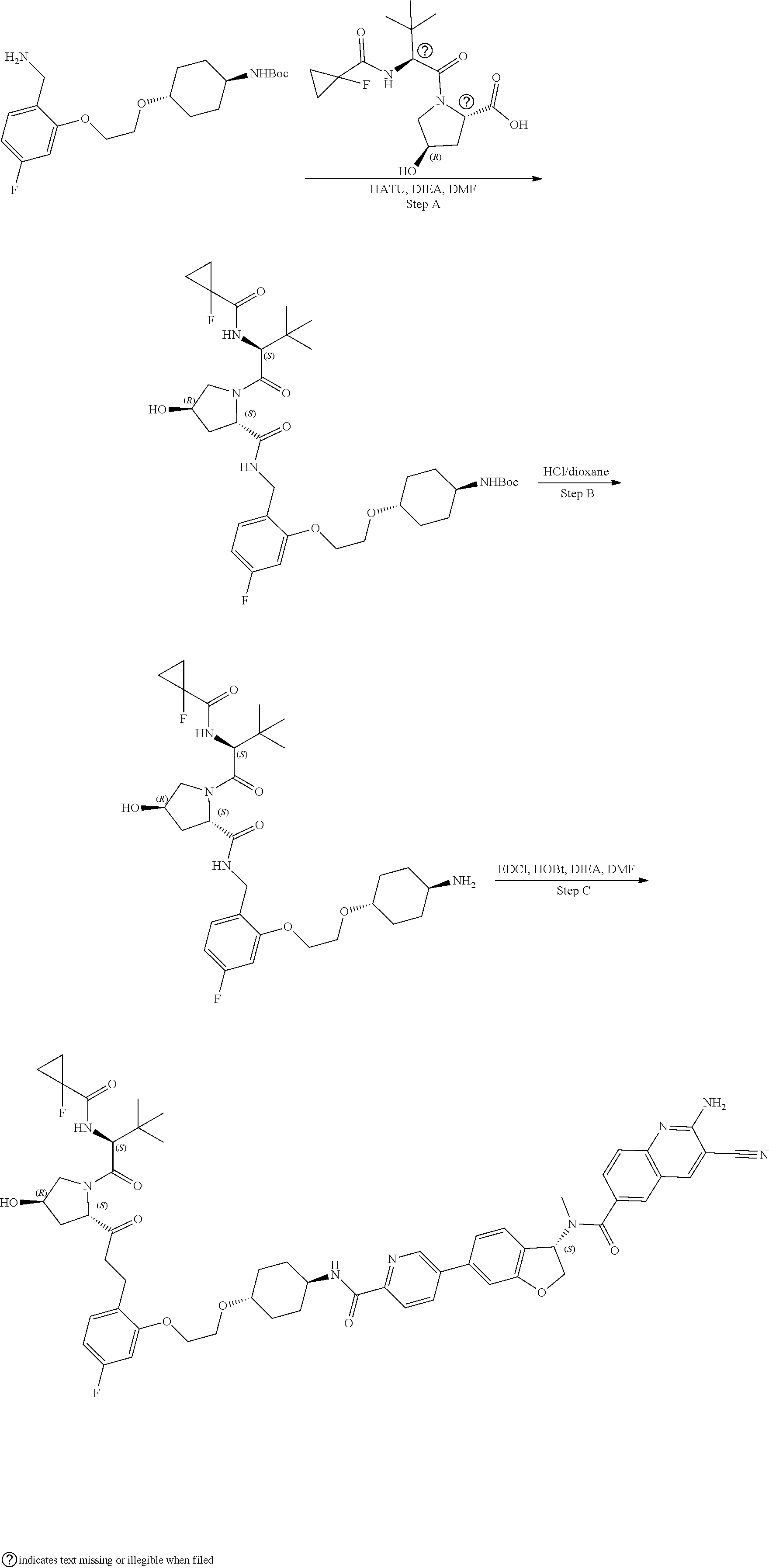

Example A219 was synthesized following the same procedures as described in Example A126 (1.8 mg, 7% yield over 2 steps) as a white solid. LC-MS (ESI): mass calcd. for C₅₆H₆₁F₂N₉O₉, 1041.46; m/z found, 1042.5 [M+H]⁺. ¹H NMR (400 MHz, DMSO-d₆) δ 8.92 (s, 1H), 8.84 (s, 1H), 8.49 (d, J=8.4 Hz, 1H), 8.42 (t, J=6.0 Hz, 1H), 8.28 (dd, J=8.2, 2.0 Hz, 1H), 8.08 (d, J=8.2 Hz, 1H), 7.98 (s, 1H), 7.83 (dd, J=8.7, 1.7 Hz, 1H), 7.58 (dd, J=17.6, 8.0 Hz, 3H), 7.39 (d, J=8.0 Hz, 1H), 7.34 (d, J=4.3 Hz, 1H), 7.32-7.24 (m, 2H), 6.90 (dd, J=11.2, 2.3 Hz, 1H), 6.66 (td, J=8.5, 2.3 Hz, 1H), 4.75 (s, 1H), 4.71-4.67 (m, 1H), 4.59 (d, J=9.2 Hz, 1H), 4.49 (t, J=8.3 Hz, 1H), 4.35 (s, 1H), 4.22 (d, J=6.0 Hz, 1H), 4.17 (d, J=5.7 Hz, 1H), 4.15-4.07 (m, 4H), 3.64-3.58 (m, 4H), 3.35 (t, J=10.3 Hz, 2H), 2.67 (s, 3H), 2.08-2.01 (m, 3H), 1.94-1.83 (m, 3H), 1.56-1.47 (m, 2H), 1.40-1.29 (m, 4H), 1.24-1.20 (m, 2H), 0.95 (d, J=5.7 Hz, 9H).

### Example A220. 2-amino-3-cyano-N-(S)-6-(6-(((1S,3S)-3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclobutyl)carbamoyl)pyridin-3-yl)-2,3-dihydrobenzofuran-3-yl)-N-methylquinoline-6-carboxamide

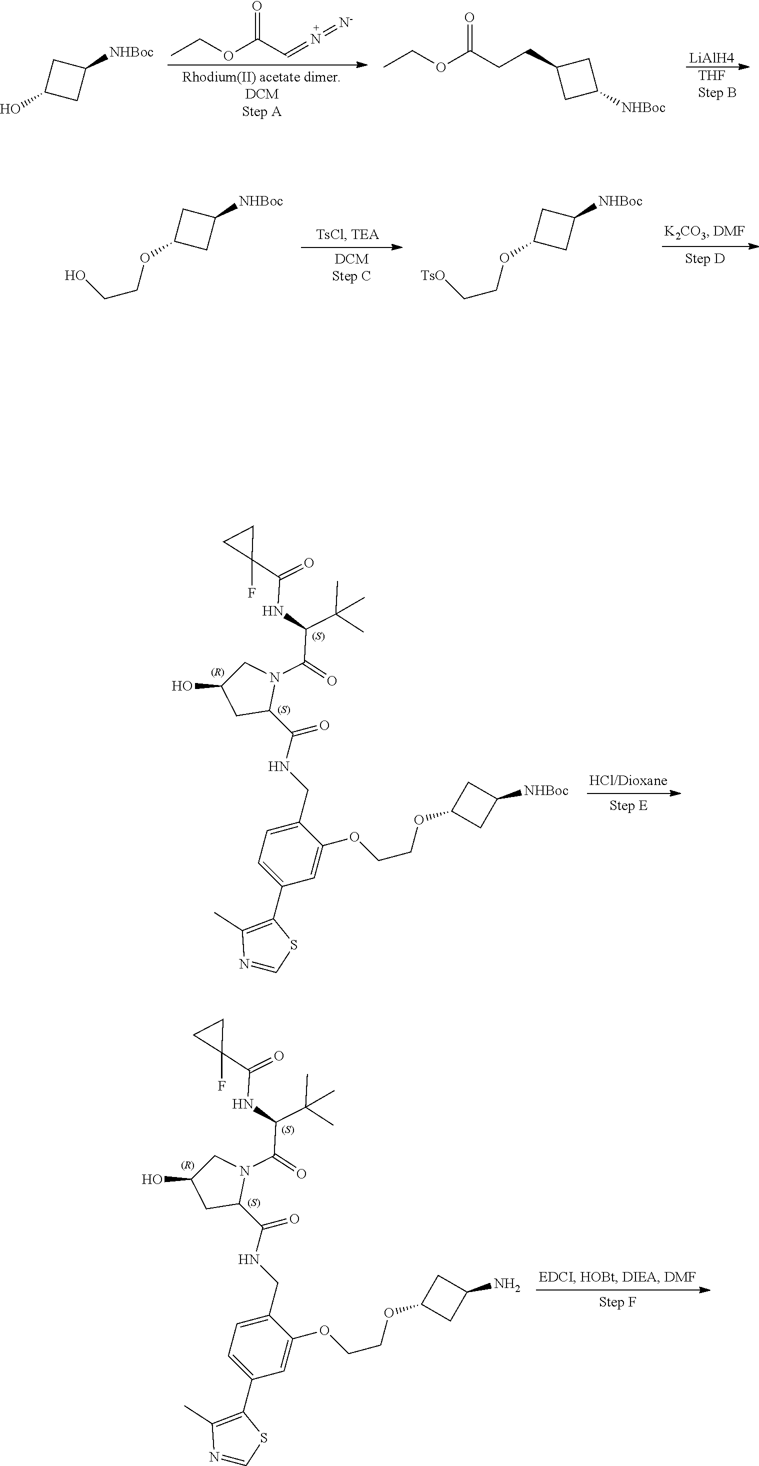

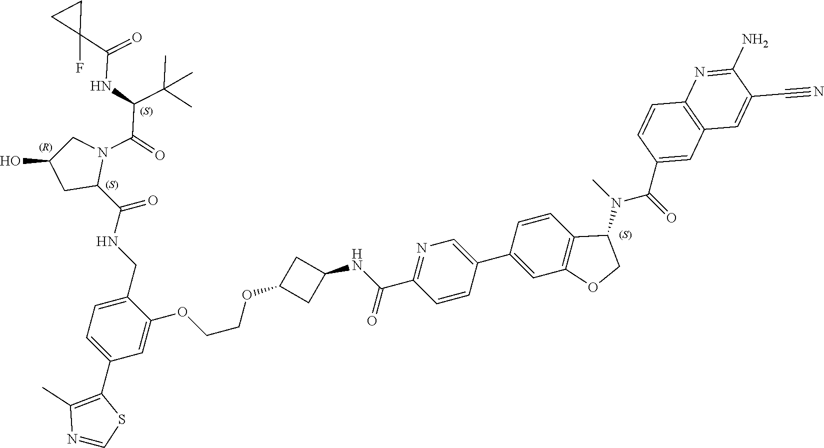

Step A: ethyl 2-((1r,3r)-3-((tert-butoxycarbonyl)amino)cyclobutoxy)acetate

To a solution of tert-butyl ((1r,3r)-3-hydroxycyclobutyl)carbamate (5.00 g, 26.7 mmol, 1.0 eq.) in DCM (50 mL) were added diacetoxyrhodium (590 mg, 1.34 mmol, 0.05 eq.) and ethyl 2-diazoacetate (12.2 g, 107 mmol, 4.0 eq.). The mixture was stirred overnight at room temperature and poured into water. The resulting solution was extracted with DCM (30 mL×2). The combined organic layers were washed with brine, dried over anhydrous sodium sulfate, concentrated under reduced pressure, and purified by silica gel chromatography (EtOAc/PE, 1/3) to afford ethyl 2-((1r,3r)-3-((tert-butoxycarbonyl)amino)cyclobutoxy)acetate (3.00 g, 41% yield) as a yellow solid.

LC-MS (ESI): mass calcd, for C13H₂₃NO₅, 273.16; m/z found, 274.2 [M+H]⁺.

Step B F: amino-3-cyano-N-((S)-6-(6-(((1S,3S)-3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclobutyl)carbamoyl)pyridin-3-yl)-2,3-dihydrobenzofuran-3-yl)-N-methylquinoline-6-carboxamide

Example A220 was synthesized following the same procedures as described in Example A23 (5.0 mg, 2% yield over 6 steps) as a white solid. LC-MS (ESI): mass calcd. for C₅₈H₆₁FN₁₀O₉S, 1092.43; m/z found, 1094.3 [M+H]⁺. ¹H NMR (400 MHz, DMSO-d₆) δ 9.06 (d, J=7.9 Hz, 1H), 9.00 (s, 1H), 8.94 (d, J=1.7 Hz, 1H), 8.87 (s, 1H), 8.51 (t, J=5.9 Hz, 1H), 8.28 (dd, J=8.2, 2.2 Hz, 1H), 8.07 (d, J=8.3 Hz, 1H), 7.99 (s, 1H), 7.88-7.81 (m, 1H), 7.62 (d, J=8.0 Hz, 2H), 7.56 (d, J=7.7 Hz, 1H), 7.41 (t, J=9.0 Hz, 2H), 7.36 (s, 1H), 7.33-7.26 (m, 1H), 7.06 (s, 1H), 6.98 (d, J=7.7 Hz, 1H), 4.71-4.67 (m, 2H), 4.61-4.54 (m, 1H), 4.56-4.54 (m, 2H), 4.36-4.34 (m, 3H), 4.25-4.24 (m, 2H), 4.22-4.20 (m, 2H), 3.73-3.68 (m, 2H), 3.68-3.58 (m, 2H), 2.68 (s, 3H), 2.47 (s, 3H), 2.45-2.38 (m, 2H), 2.36-2.28 (m, 2H), 2.13-2.03 (m, 1H), 2.00-1.87 (m, 1H), 1.41-1.31 (m, 2H), 1.25-1.19 (m, 2H), 0.96 (s, 9H).

### Example A221. 2-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-3-isopropoxy-N-methylquinoline-6-carboxamide

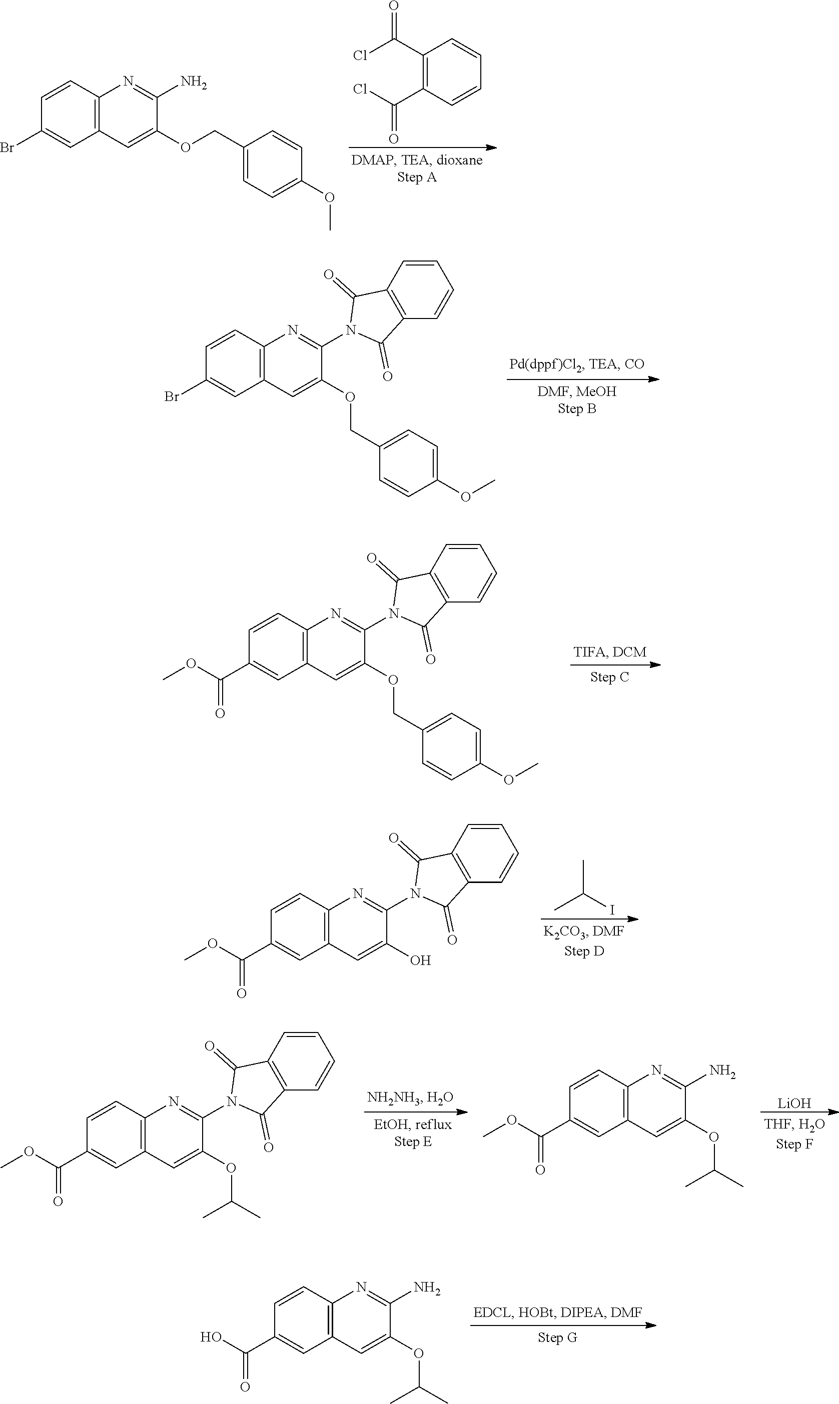

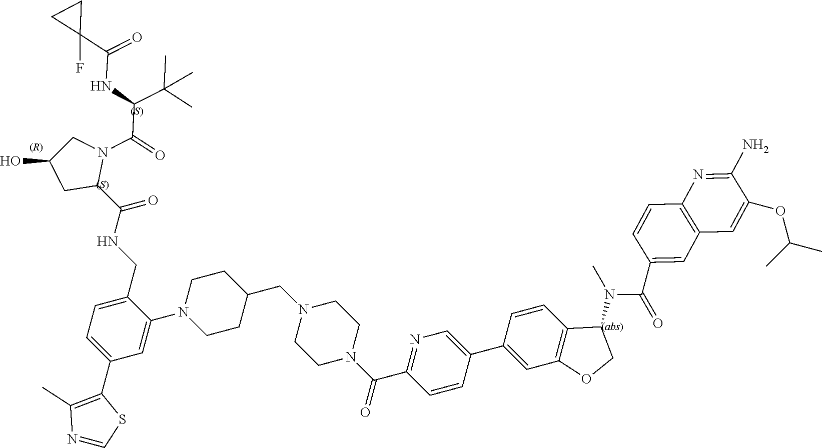

Step A: 2-(6-bromo-3-(4-methoxybenzyl)oxy) quinolin-2-ylisoindoline-1,3-dione

To a solution of 6-bromo-3-((4-methoxybenzyl)oxy) quinolin-2-amine (1.74 g, 4.84 mmol, 1.0 eq.) in 1,4-Dioxane (20 mL) was added TEA (3.38 mL, 24.2 mmol, 5.0 eq.), DMAP (592 mg, 4.84 mmol, 1.0 eq.) and phthaloyl dichloride (1.5 g, 7.27 mmol, 1.5 eq.) under N₂. The mixture was stirred at 90° C. for 16 hours to give yellow solution. The mixture was concentrated. The residue was purified by flash chromatography (100-200 mesh silica gel, 0%-100% EtOAc in PE) to afford 2-(6-bromo-3-((4-methoxybenzyl)oxy) quinolin-2-ylisoindoline-1,3-dione (1.7 g, 68% yield) as a yellow solid. LC-MS (ESI): mass calcd. For C₂₅H₁₇BrN₂O₄, 489.3; m/z found, 489.0/491.0 [M+H−56]⁺.

Step B: tert-butyl (1r,4r)-4-(3-(2-(((tert-butylsulfinyl)amino)methyl)-5-(4-methylthiazol-5-yl)phenyl) prop-2-yn-1-yl)oxy)cyclohexyl)carbamate

To a solution of 2-(6-bromo-3-((4-methoxybenzyl)oxy) quinolin-2-yl) isoindoline-1,3-dione (400 mg, 817 μmol, 1.0 eq.) in DMF (10 mL) and MeOH (2 mL) was added 1,1′-Bis(diphenylphosphino) ferrocene-palladium (II) dichloride (59.8 mg, 81.7 μmol, 0.1 eq.) and TEA (245 mg, 2.45 mmol, 3.0 eq.). The mixture was degassed with CO for 3 times and stirred at 100° C. using a CO balloon for 2 hours. The reaction mixture was quenched water, extracted with EtOAc (30 mL×2) and the layers were separated. The organic layer was washed with brine, dried over Na₂SO₄, filtered and concentrated. The residue was purified by flash chromatography (100-200 mesh silica gel, 0%-100% EA in PE) to afford methyl 2-(1,3-dioxoisoindolin-2-yl)-3-((4-methoxybenzyl)oxy) quinoline-6-carboxylate (160 mg, 40% yield) as a yellow solid. LC-MS (ESI): mass calcd. For C₂₇H₂₀N₂O₆, 468.5; m/z found, 469.1 [M+H]⁺.

Step C: methyl 2-(1,3-dioxoisoindolin-2-yl)-3-hydroxyquinoline-6-carboxylate

A mixture of methyl 2-(1,3-dioxoisoindolin-2-yl)-3-((4-methoxybenzyl)oxy) quinoline-6-carboxylate (200 mg, 426 μmol, 1.0 eq.) in DCM (5 mL) and TFA (2 mL) was stirred at room temperature for 1 hour to give yellow solution. The residue was concentrated to afford methyl 2-(1,3-dioxoisoindolin-2-yl)-3-hydroxyquinoline-6-carboxylate (100 mg, 64% yield) as a yellow oil. LC-MS (ESI): mass calcd. For C₁₉H₁₂N₂O₅, 348.3; m/z found, 349.1 [M+H]⁺.

Step D: tert-butyl ((1r,4r)-4-(3-(2-(aminomethyl)-5-(4-methylthiazol-5-yl)phenyl) propoxy)cyclohexyl)carbamate

To a solution of methyl 2-(1,3-dioxoisoindolin-2-yl)-3-hydroxyquinoline-6-carboxylate (100 mg, 287 μmol, 1.0 eq.) in DMF (10 mL) was added K₂CO₃ (119 mg, 861 μmol, 3.0 eq.) and 2-iodopropane (97 mg, 574 μmol, 2.0 eq.) under N₂. The mixture was stirred at 100° C. for 16 hours to give yellow solution. The reaction mixture was quenched water, extracted with EtOAc (30 mL×2) and the layers were separated. The organic layer was washed with brine, dried over Na₂SO₄, filtered and concentrated. The residue was purified by flash chromatography (100-200 mesh silica gel, 0%-100% EtOAc in PE) to afford methyl 2-(1,3-dioxoisoindolin-2-yl)-3-isopropoxyquinoline-6-carboxylate (80 mg, 68% yield) as a yellow solid. LC-MS (ESI): mass calcd. For C₂₂H₁₈N₂O₅, 390.1; m/z found, 391.1 [M+H]⁺.

Step E: methyl 2-amino-3-isopropoxyquinoline-6-carboxylate

A solution of methyl 2-(1,3-dioxoisoindolin-2-yl)-3-isopropoxyquinoline-6-carboxylate (40 mg, 102 μmol, 1.0 eq.) in EtOH (6 mL) and NH₂—NH₂ H₂O (1 mL) was stirred at 85° C. for 2 hours to give yellow solution. The mixture was concentrated. The residue was purified by flash chromatography (100-200 mesh silica gel, 0%-100% EtOAc in PE) to afford methyl 2-amino-3-isopropoxyquinoline-6-carboxylate (15 mg, 53% yield) as a yellow solid. LC-MS (ESI): mass calcd. For C₁₄H₁₆N₂O₃, 260.1; m/z found, 261.1 [M+H]⁺.

Step F-G: 2-amino-N-(S)-6-16-(4-((1-(2-(((25, 4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-3-isopropoxy-N-methylquinoline-6-carboxamide

Example A221 was synthesized following the same procedures as described in Example A107 (2.7 mg, 13% yield over 2 steps) as a white solid. LC-MS (ESI): mass calcd. for C₆₄H₇₆FN₁₁O₈S, 1178.4; m/z found, 1179.4 [M+H]⁺. ¹H NMR (400 MHz, DMSO-d₆) δ 8.97 (s, 1H), 8.91 (s, 1H), 8.48 (t, J=6.0 Hz, 1H), 8.25-8.17 (m, 1H), 7.77 (s, 1H), 7.65 (d, J=8.2 Hz, 1H), 7.53 (d, J=7.5 Hz, 1H), 7.49-7.36 (m, 5H), 7.33 (s, 1H), 7.28 (d, J=7.3 Hz, 1H), 7.10 (d, J=9.8 Hz, 2H), 6.48 (s, 2H), 5.16 (d, J=3.5 Hz, 1H), 4.81-4.71 (m, 2H), 4.71-4.64 (m, 1H), 4.60 (d, J=9.4 Hz, 1H), 4.48 (t, J=8.2 Hz, 1H), 4.45-4.24 (m, 3H), 3.73-3.54 (m, 4H), 3.53-3.43 (m, 2H), 3.36-3.33 (m, 6H), 3.12-3.01 (m, 2H), 2.80-2.58 (m, 6H), 2.45 (s, 3H), 2.42-2.35 (m, 2H), 2.31-2.22 (m, 2H), 2.12-1.99 (m, 2H), 1.98-1.87 (m, 2H), 1.87-1.77 (m, 2H), 1.78-1.61 (m, 1H), 1.36 (d, 6H), 0.96 (s, 9H).

### Example A222. 2-amino-3-cyano-N-((3S)-6-(6-((2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclobutyl)carbamoyl) pyridin-3-yl)-2,3-dihydrobenzofuran-3-yl)-N-methylquinoline-6-carboxamide

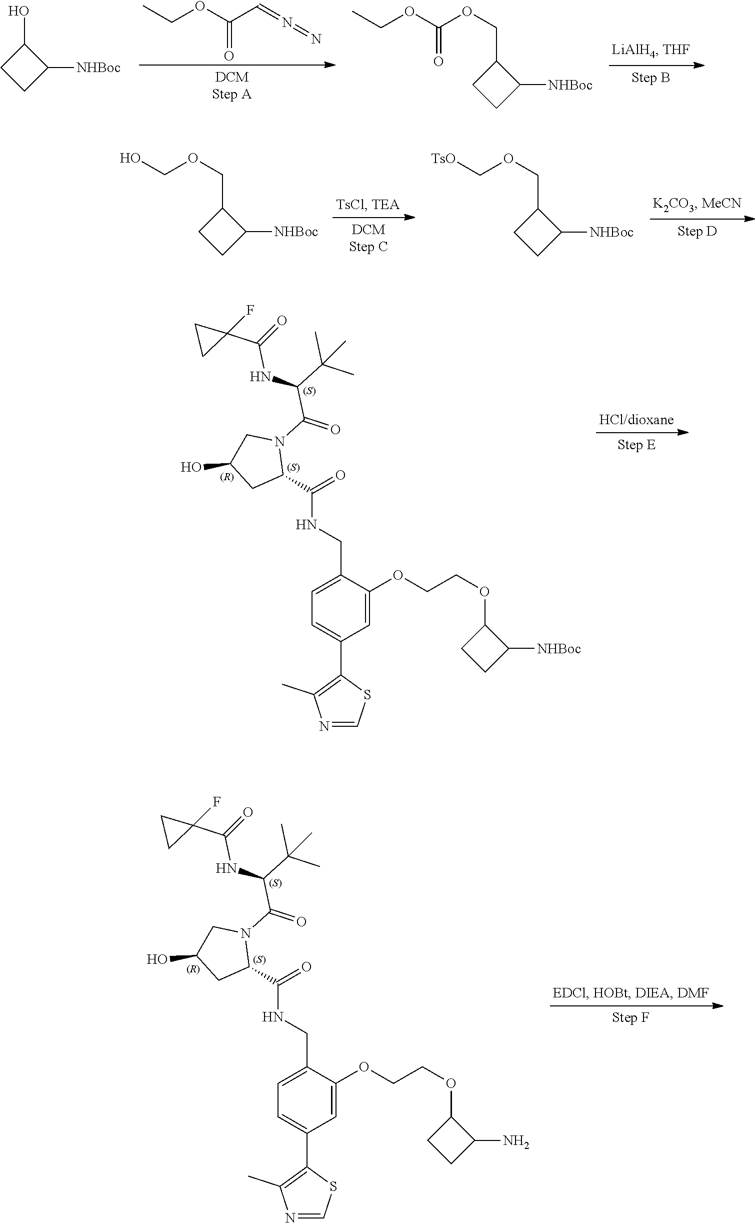

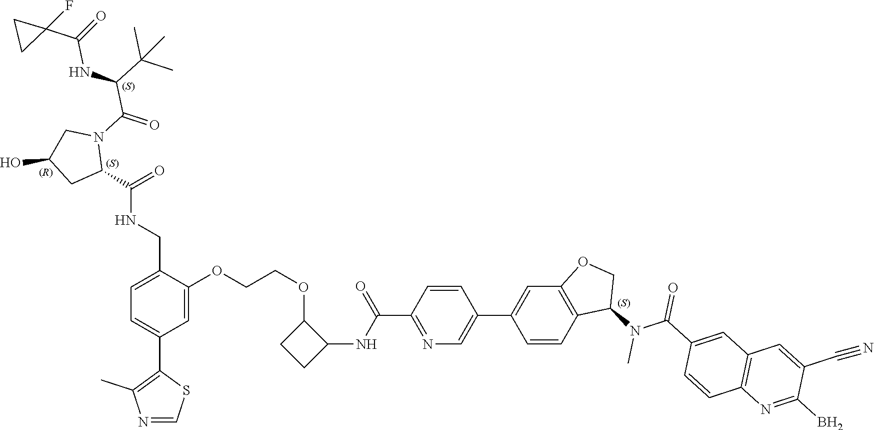

Example A222 was synthesized following the same procedures as described in Example A220 (2.7 mg, 2% yield over 6 steps) as a white solid. LC-MS (ESI): mass calcd. for C₅₈H₆₁FN₁₀O₉S, 1092.43; m/z found, 1094.3 [M+H]⁺. ¹H NMR (400 MHz, DMSO-d₆) δ 8.97-8.92 (m, 1H), 8.91-8.80 (m, 2H), 8.72 (d, J=7.7 Hz, 1H), 8.52-8.40 (m, 1H), 8.23 (d, J=8.1 Hz, 1H), 8.04 (d, J=8.1 Hz, 1H), 7.97 (s, 1H), 7.82 (d, J=8.7 Hz, 1H), 7.59 (d, J=8.5 Hz, 1H), 7.54 (d, J=8.1 Hz, 1H), 7.38 (dd, J=11.0, 7.8 Hz, 3H), 7.32-7.24 (m, 2H), 6.97-6.90 (m, 2H), 4.72-4.66 (m, 1H), 4.61-4.47 (m, 3H), 4.38-4.28 (m, 3H), 4.27-4.14 (m, 4H), 3.81-3.78 (m, 3H), 3.47-3.41 (m, 2H), 2.68 (s, 3H), 2.43-2.39 (m, 3H), 2.18-2.06 (m, 3H), 2.02-1.93 (m, 2H), 1.90-1.80 (m, 1H), 1.40-1.32 (m, 2H), 1.24-1.19 (m, 2H), 0.94 (s, 9H).

### Example A223. 2-amino-3-cyano-N-((S)-6-(6-((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) propyl)carbamoyl)pyridin-3-yl)-2,3-dihydrobenzofuran-3-yl)-N-methylquinoline-6-carboxamide

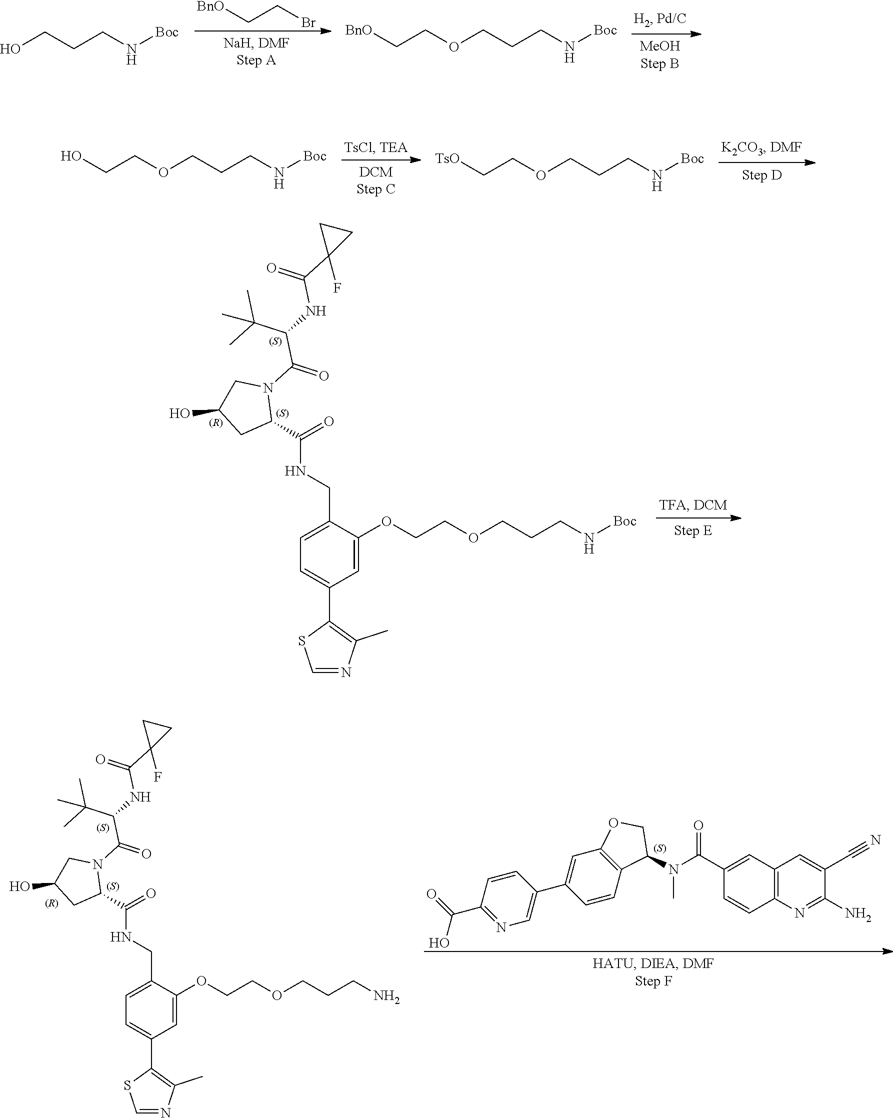

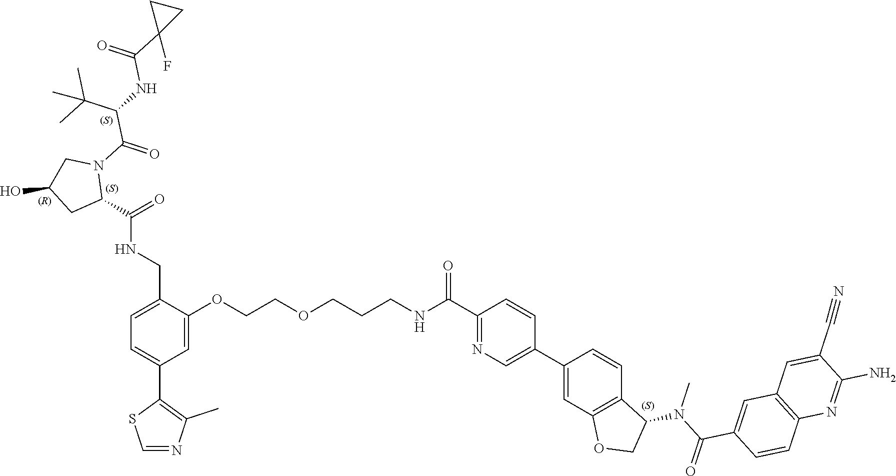

Example A223 was synthesized following the same procedures as described in Example A29 (1.6 mg, 2% yield over 6 steps) as a white solid. LC-MS (ESI): mass calcd. for C₅₇H₆₁FN₁₀O₇S, 1080.4; m/z found, 1082 [M+H]⁺. ¹H NMR (400 MHz, DMSO-d₆) δ 8.95 (s, 1H), 8.86 (m, 1H), 8.80 (m, 2H), 8.47 (t, J=6.0 Hz, 1H), 8.23 (dd, J=8.2, 2.0 Hz, 1H), 8.06 (d, J=8.1 Hz, 1H), 7.97 (s, 1H), 7.82 (d, J=8.7 Hz, 1H), 7.60 (d, J=7.4 Hz, 1H), 7.52 (d, J=7.7 Hz, 1H), 7.41 (t, J=11.9 Hz, 2H), 7.33 (d, J=8.1 Hz, 1H), 7.28 (m, 2H), 7.04 (s, 1H), 6.95 (d, J=7.7 Hz, 1H), 4.84-4.65 (m, 2H), 4.59 (d, J=9.1 Hz, 1H), 4.51 (t, J=8.2 Hz, 1H), 4.36-4.28 (m, 2H), 4.24-4.18 (m, 3H), 3.79 (s, 2H), 3.64-3.58 (m, 5H), 3.44-3.40 (m, 2H), 2.66 (s, 3H), 2.43 (s, 3H), 2.11-2.04 (m, 1H), 1.94-1.87 (m, 1H), 1.85-1.79 (m, 2H), 1.41-1.36 (m, 1H), 1.35-1.31 (m, 1H), 1.23-1.19 (m, 2H), 0.94 (s, 9H).

### Example A224. 2-amino-3-cyano-N-((3S)-6-(6-((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclopentyl)carbamoyl) pyridin-3-yl)-2,3-dihydrobenzofuran-3-yl)-N-methylquinoline-6-carboxamide

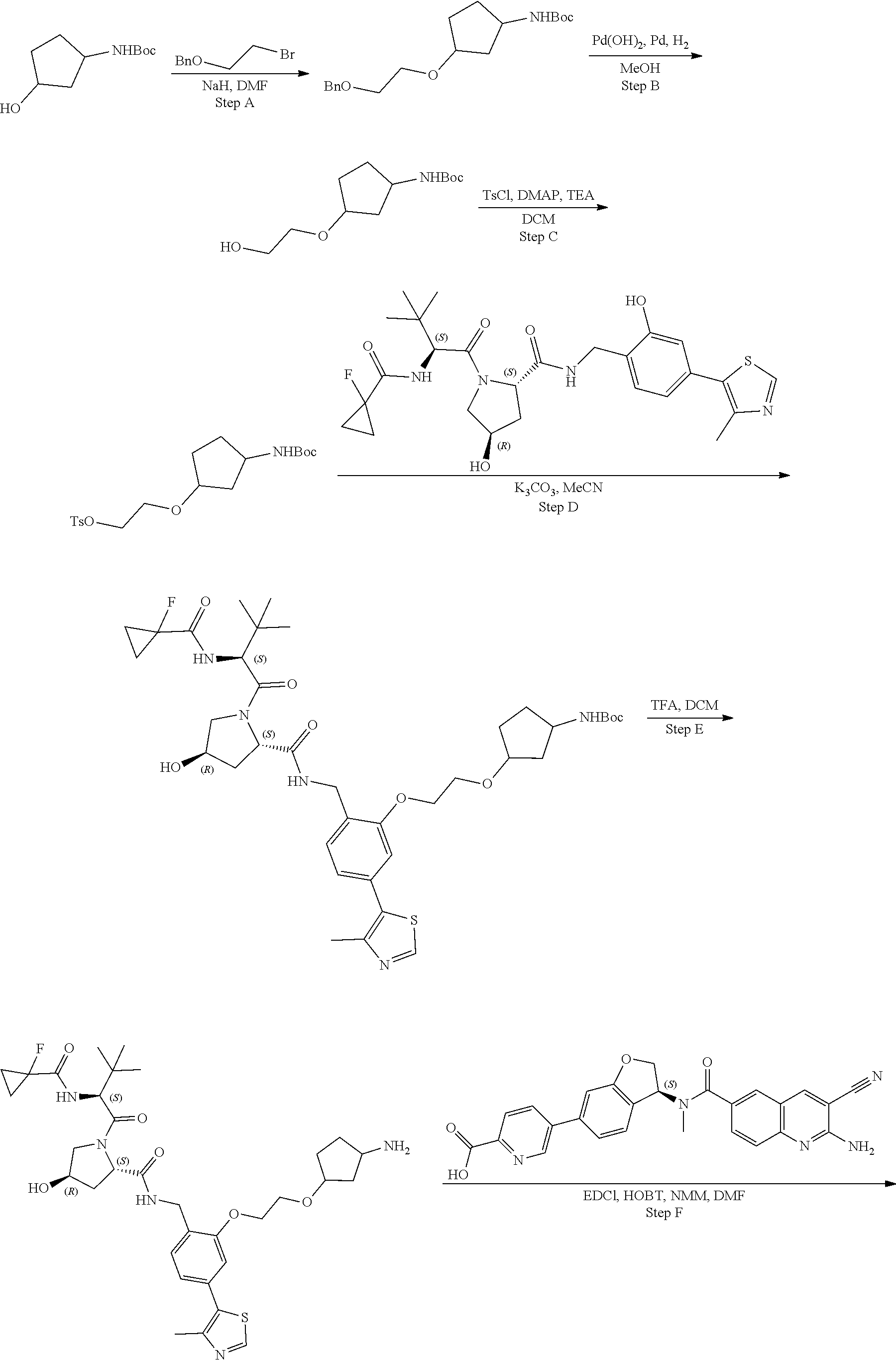

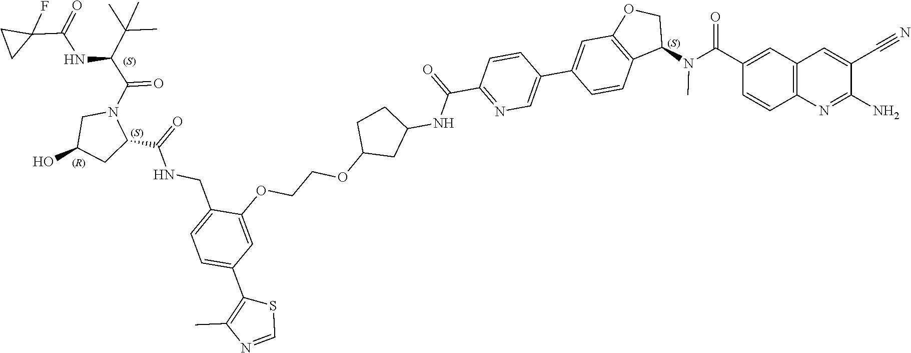

Example A224 was synthesized following the same procedures as described in Example A29 (2 mg, 1% yield over 6 steps) as a white solid. LC-MS (ESI): mass calcd, for C₅₉H₆₃FN₁₀O₉S, 1106.4/z found, 1107.1 [M+H]⁺. ¹H NMR (400 MHz, DMSO-d₆) δ 8.90 (s, 1H), 8.83 (s, 1H), 8.71 (d, J=8.9 Hz, 1H), 8.52 (s, 1H), 8.44 (d, J=3.7 Hz, 1H), 8.22-8.17 (m, 1H), 8.04 (d, J=8.2 Hz, 1H), 7.97 (s, 1H), 7.86-7.79 (m, 1H), 7.60 (d, J=7.2 Hz, 1H), 7.46 (d, J=7.7 Hz, 2H), 7.39-7.34 (m, 1H), 7.29-7.17 (m, 3H), 7.05 (s, 1H), 6.92 (d, J=7.7 Hz, 1H), 6.36 (s, 1H), 4.68-4.65 (m, 2H), 4.59-4.57 (m, 1H), 4.51-4.47 (m, 2H), 4.44-4.39 (m, 2H), 4.33-4.31 (m, 1H), 4.25-4.20 (m, 3H), 4.14-4.10 (m, 1H), 3.83-3.76 (m, 2H), 3.64-3.55 (m, 2H), 2.66 (s, 3H), 2.38 (s, 3H), 2.07-1.95 (m, 3H), 1.92-1.79 (m, 4H), 1.76-1.69 (m, 1H), 1.40-1.30 (m, 2H), 1.24-1.18 (m, 2H), 0.93 (s, 9H).

### Example A225. 2-amino-3-cyano-N-((3S)-6-(6-((1-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)piperidin-3-yl)carbamoyl)pyridin-3-yl)-2,3-dihydrobenzofuran-3-yl)-N-methylquinoline-6-carboxamide

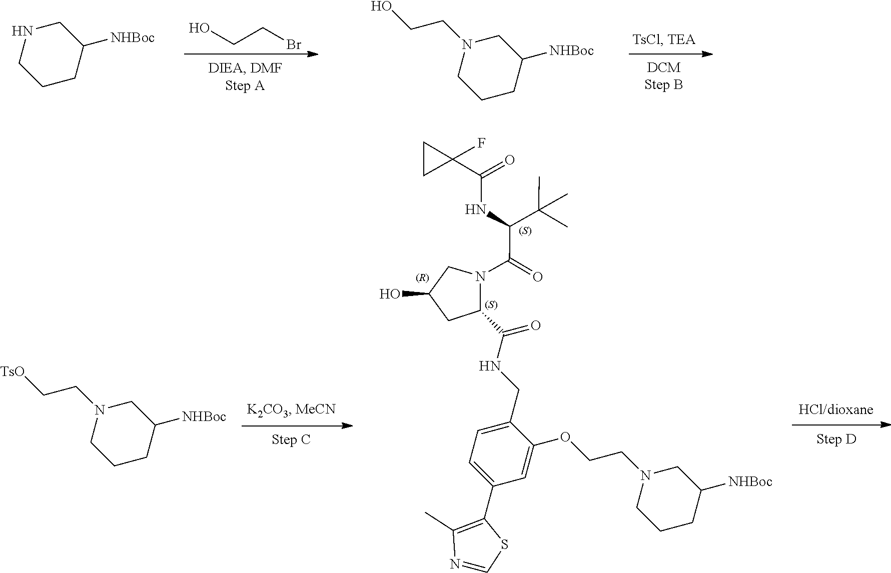

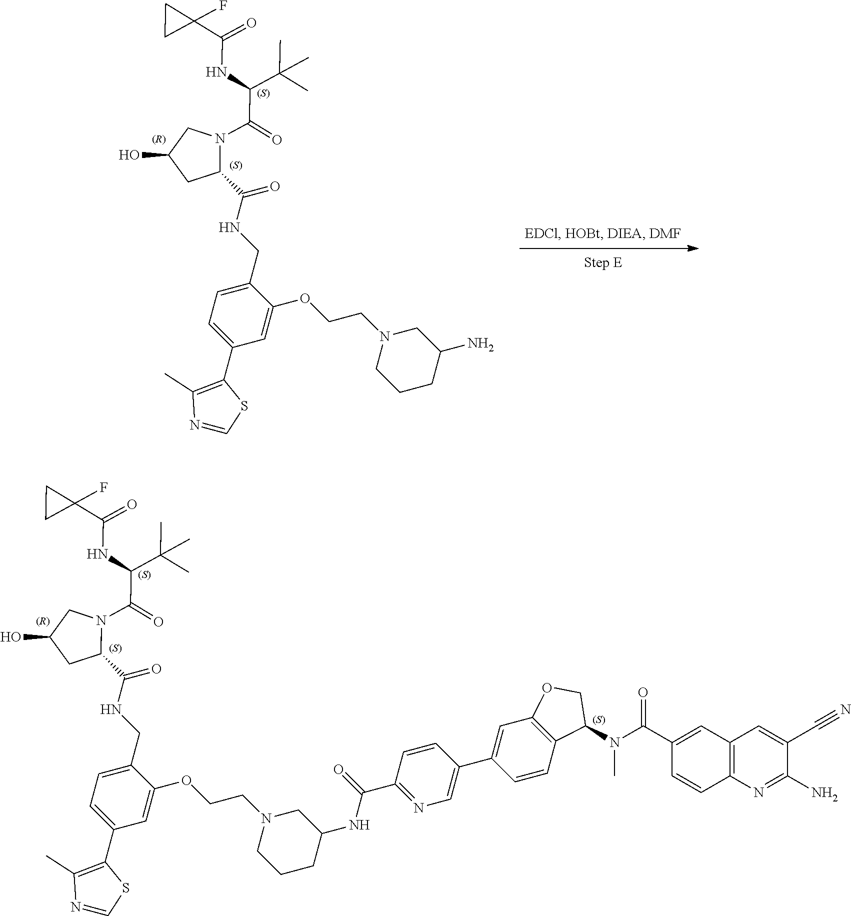

Example A225 was synthesized following the same procedures as described in Example A29 (3.6 mg, 2% yield over 5 steps) as a white solid. LC-MS (ESI): mass calcd. for C₅₉H₆₄FN1O₈S, 1106.29/z found, 1107.5 [M+H]⁺. ¹H NMR (400 MHz, DMSO-d₆) δ 9.65 (s, 1H), 9.16-9.03 (m, 1H), 9.00 (s, 1H), 8.98-8.92 (m, 1H), 8.78 (s, 1H), 8.65-8.43 (m, 1H), 8.35-8.26 (m, 1H), 8.14-8.06 (m, 1H), 7.95 (s, 1H), 7.79 (dd, J=8.7, 1.9 Hz, 1H), 7.57 (d, J=7.6 Hz, 2H), 7.48-7.34 (m, 3H), 7.30-7.19 (m, 3H), 7.09-6.99 (m, 2H), 5.32 (t, J=4.7 Hz, 1H), 4.73-4.57 (m, 2H), 4.54-4.42 (m, 3H), 4.37-4.27 (m, 4H), 3.72-3.59 (m, 7H), 3.16-2.97 (m, 3H), 2.67 (s, 3H), 2.47 (s, 3H), 2.04-1.97 (m, 3H), 1.92-1.82 (m, 3H), 1.25-1.22 (m, 4H), 0.93 (s, 9H).

### Example A226. 2-amino-3-cyano-N-((S)-6-(6-((2-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl)benzyl)carbamoyl)

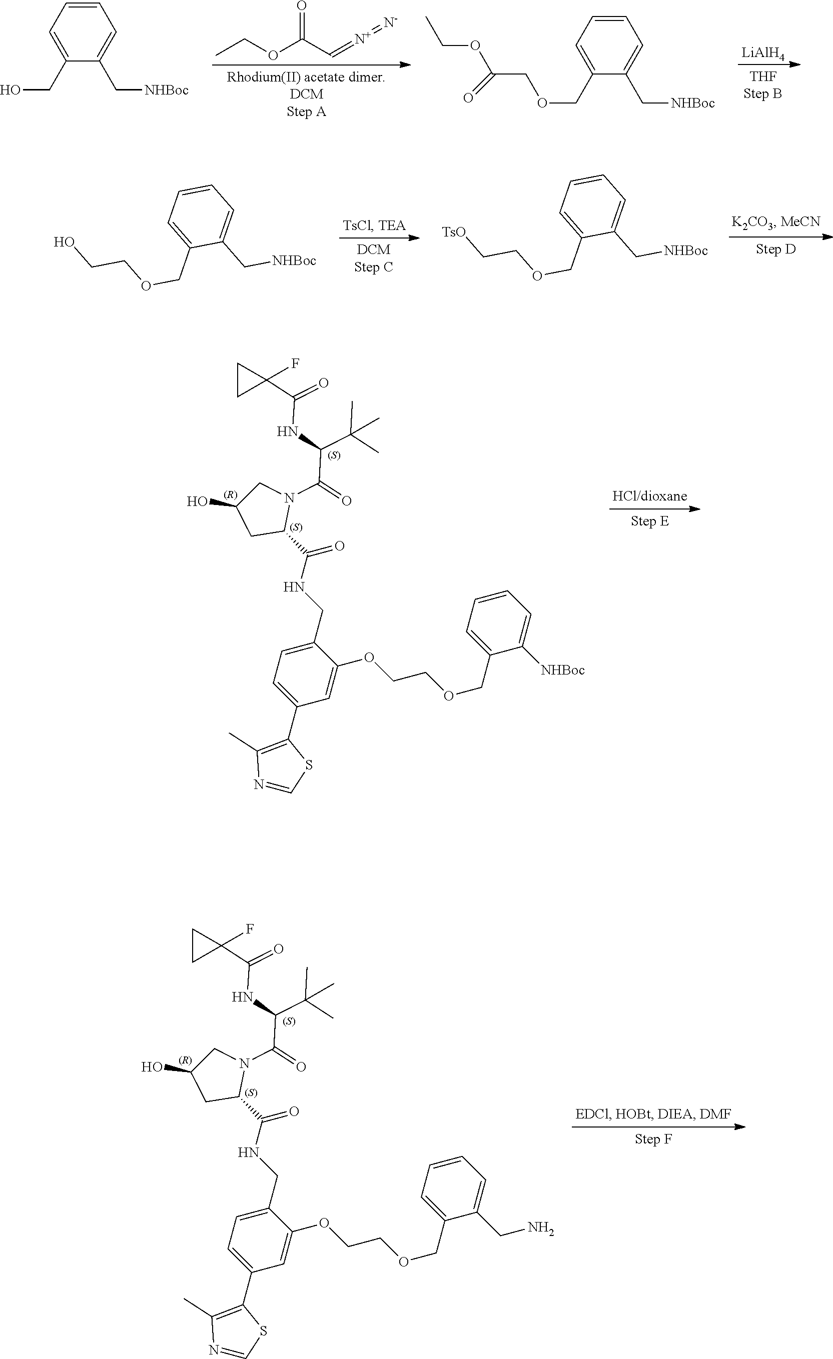

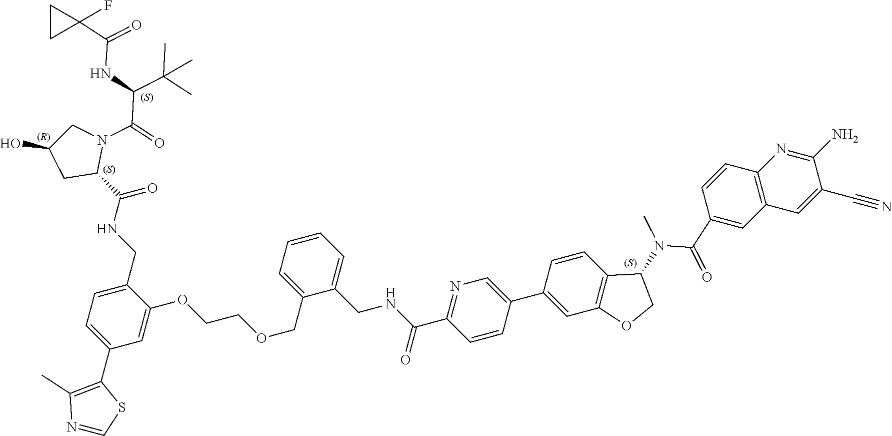

Example A226 was synthesized following the same procedures as described in Example A220 (14 mg, 8% yield over 6 steps) as a white solid. LC-MS (ESI): mass calcd. For C₆₂H₆₃FN₁₀O₉S, 1142.45/z found, 1144 [M+H]⁺. ¹H NMR (400 MHz, DMSO-d₆) δ 9.14 (t, J=6.2 Hz, 1H), 8.92 (s, 1H), 8.87-8.73 (m, 2H), 8.46 (t, J=5.9 Hz, 1H), 8.26-8.20 (m, 1H), 8.07 (d, J=8.2 Hz, 1H), 7.98 (s, 1H), 7.82 (dd, J=8.7, 1.8 Hz, 1H), 7.66-7.56 (m, 1H), 7.53-7.44 (m, 2H), 7.41-7.21 (m, 9H), 7.03 (s, 1H), 6.93 (d, J=7.8 Hz, 1H), 4.76 (s, 2H), 4.70-4.66 (m, 1H), 4.63-4.56 (m, 3H), 4.53-4.48 (m, 1H), 4.35-4.28 (m, 4H), 4.25-4.20 (m, 1H), 3.92-3.90 (m, 2H), 3.66-3.59 (m, 2H), 2.66 (s, 3H), 2.38 (s, 3H), 2.07-2.01 (m, 1H), 1.92-1.83 (m, 1H), 1.41-1.31 (m, 2H), 1.26-1.18 (m, 4H), 0.94 (s, 9H).

### Example A227. 2-amino-3-cyano-N-((S)-6-(6-(((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclobutyl)methyl) carbamoyl)pyridin-3-yl)-2,3-dihydrobenzofuran-3-yl)-N-methylquinoline-6-carboxamide

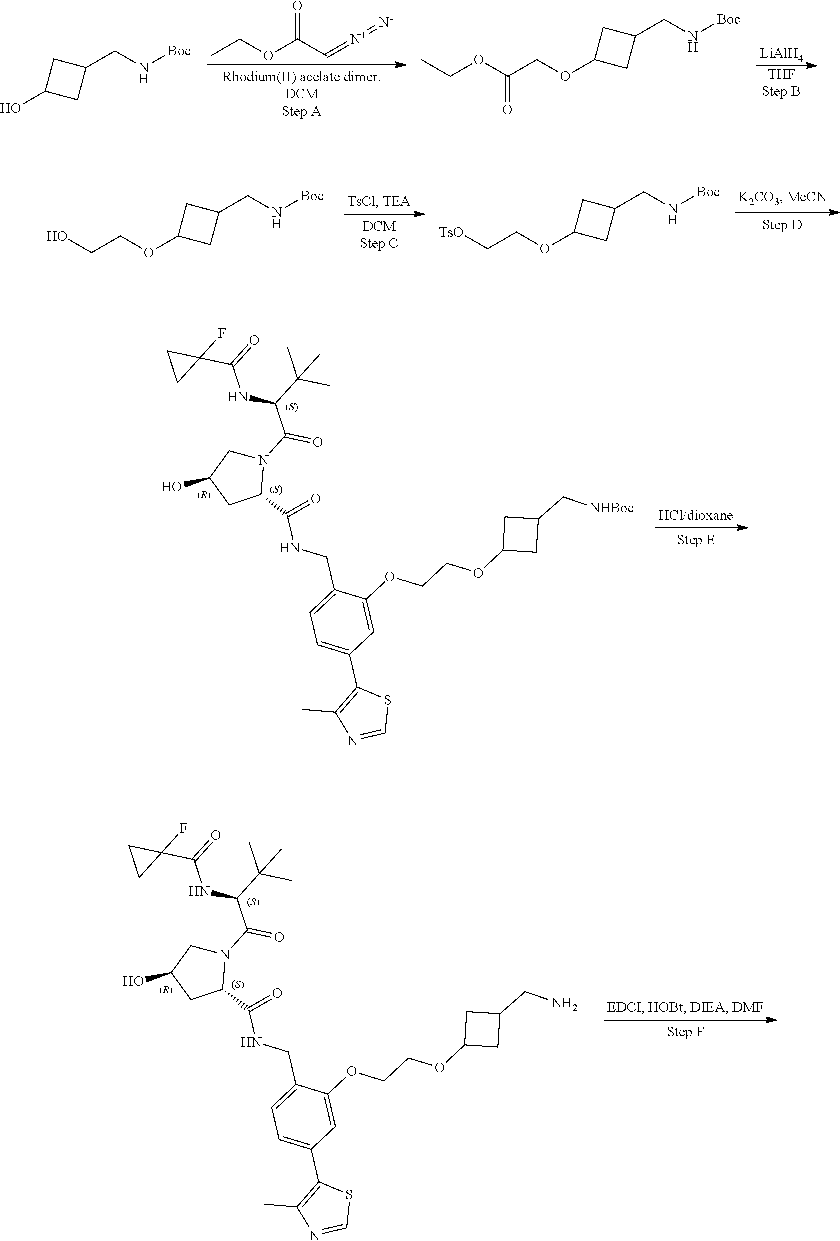

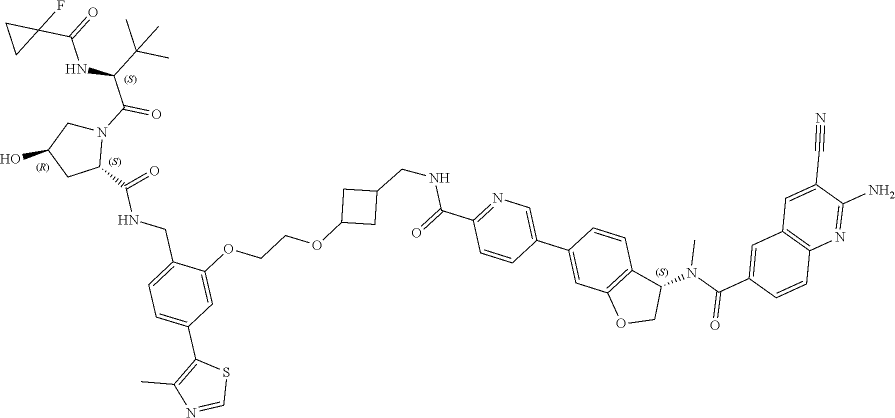

Example A227 was synthesized following the same procedures as described in Example A220 (2.5 mg, 1% yield over 6 steps) as a white solid. LC-MS (ESI): mass calcd. for C₅₉H₆₃FN₁₀O₉S, 1106.4/z found, 1107.4 [M+H]⁺. ¹H NMR (400 MHz, DMSO-d₆) δ 8.98 (s, 1H), 8.93 (s, 1H), 8.84 (s, 2H), 8.49 (t, J=5.7 Hz, 1H), 8.25 (dd, J=16.5, 14.3 Hz, 1H), 8.08 (d, J=8.1 Hz, 1H), 7.98 (s, 1H), 7.83 (d, J=8.7 Hz, 1H), 7.61-7.55 (m, 4H), 7.40 (d, J=7.7 Hz, 2H), 7.35 (s, 1H), 7.29 (d, J=9.2 Hz, 1H), 7.03 (s, 1H), 6.96 (d, J=7.8 Hz, 1H), 4.71-4.66 (m, 1H), 4.60-4.56 (m, 1H), 4.54-4.49 (m, 1H), 4.36-4.32 (m, 2H), 4.31-4.26 (m, 2H), 4.25-4.22 (m, 2H), 3.96-3.85 (m, 1H), 3.67-3.56 (m, 5H), 3.46-3.26 (m, 2H), 2.67 (s, 3H), 2.45 (s, 3H), 2.40-2.26 (m, 2H), 2.15-2.01 (m, 2H), 2.03-1.87 (m, 2H), 1.72-1.54 (m, 1H), 1.40-1.25 (m, 2H), 1.23-1.16 (m, 2H), 0.95 (s, 9H).

### Example A228. 2-amino-3-cyano-N-((S)-6-(6-(((1S,4S)-4-((3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl) prop-2-yn-1-yl)oxy)cyclohexyl) carbamoyl)pyridin-3-yl)-2,3-dihydrobenzofuran-3-yl)-N-methylquinoline-6-carboxamide

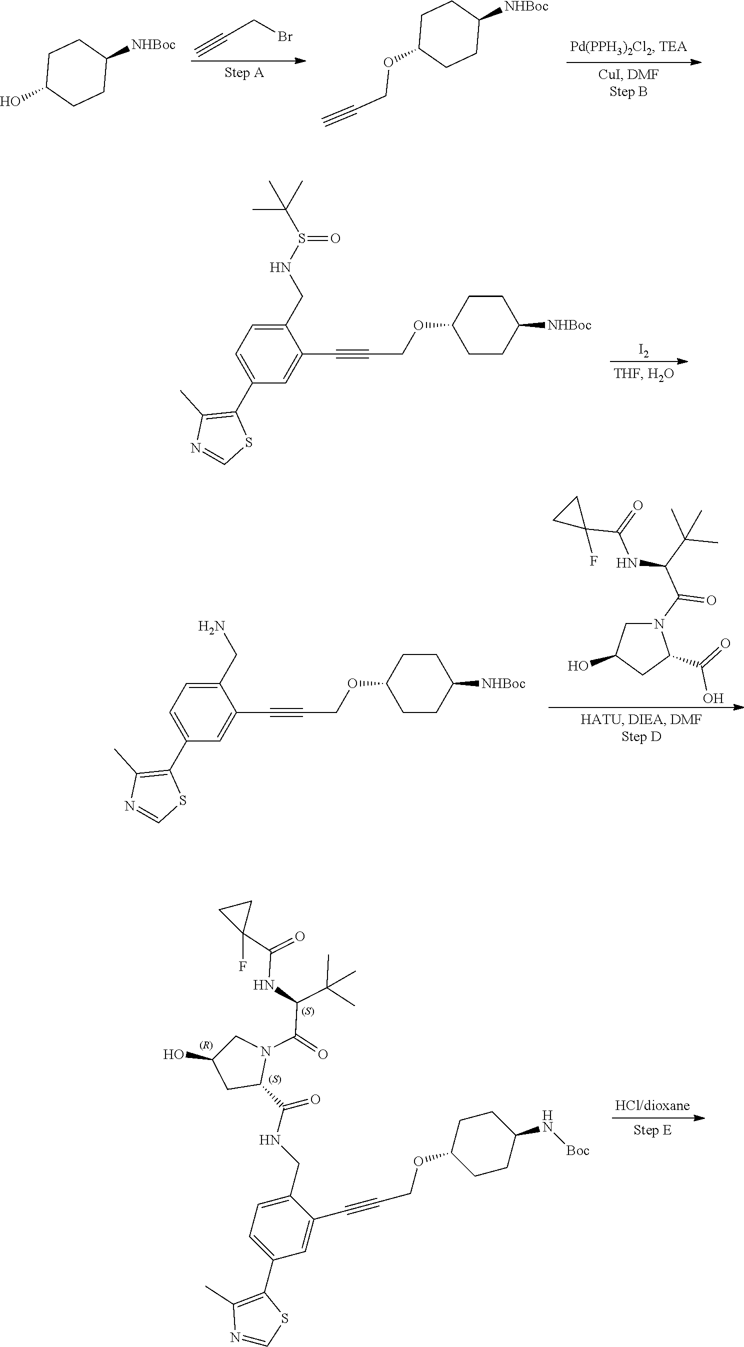

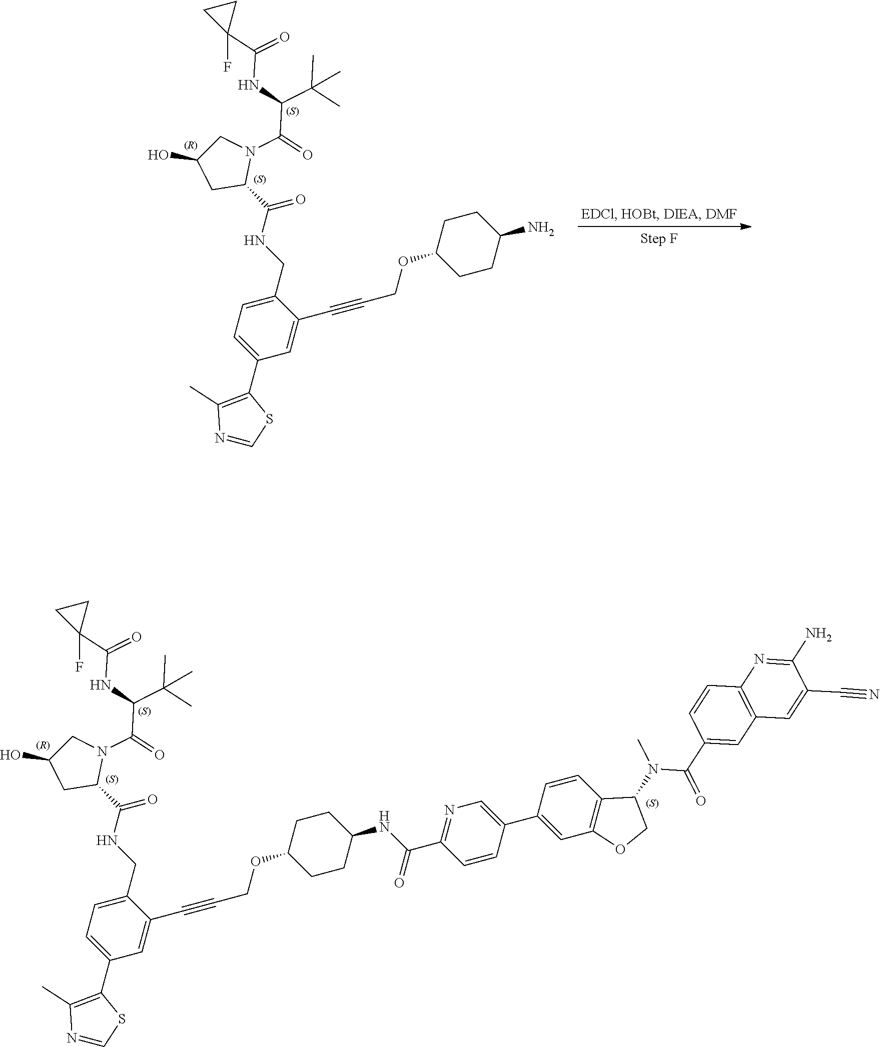

Example A228 was synthesized following the same procedures as described in Example A150 (11.2 mg, 10% yield over 6 steps) as a white solid. LC-MS (ESI): mass calcd. for C₆₁H₆₃FN1008S, 1114.45; m/z found, 1115.8 [M+H]⁺. ¹H NMR (400 MHz, DMSO-d₆) δ 9.02 (s, 1H), 8.93-8.91 (m, 1H), 8.84 (s, 1H), 8.73-8.67 (m, 1H), 8.48 (d, J=8.5 Hz, 1H), 8.28 (dd, J=8.2, 2.1 Hz, 1H), 8.08 (d, J=8.2 Hz, 1H), 7.98 (s, 1H), 7.83 (dd, J=8.7, 1.8 Hz, 1H), 7.63-7.46 (m, 6H), 7.45-7.42 (m, 1H), 7.39 (d, J=7.9 Hz, 1H), 7.34 (s, 1H), 7.31-7.27 (m, 1H), 4.71-4.66 (m, 2H), 4.60 (d, J=9.2 Hz, 1H), 4.55-4.45 (m, 5H), 4.43-4.38 (m, 1H), 4.36-4.34 (m, 1H), 3.86-3.79 (m, 1H), 3.68-3.59 (m, 2H), 3.56-3.50 (m, 1H), 2.67 (s, 3H), 2.46 (s, 3H), 2.13-2.06 (m, 3H), 1.96-1.85 (m, 3H), 1.57-1.48 (m, 2H), 1.39-1.31 (m, 4H), 1.23-1.19 (m, 2H), 0.96 (s, 9H).

### Example A229. 2-amino-3-cyclopropyl-N-((S)-6-(6-(4-((1-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-1,2,3,6-tetrahydropyridin-4-yl)methyl)piperazine-1-carbonyl)pyridin-3-yl)-2,3-dihydrobenzofuran-3-yl)-N-methylquinoline-6-carboxamide

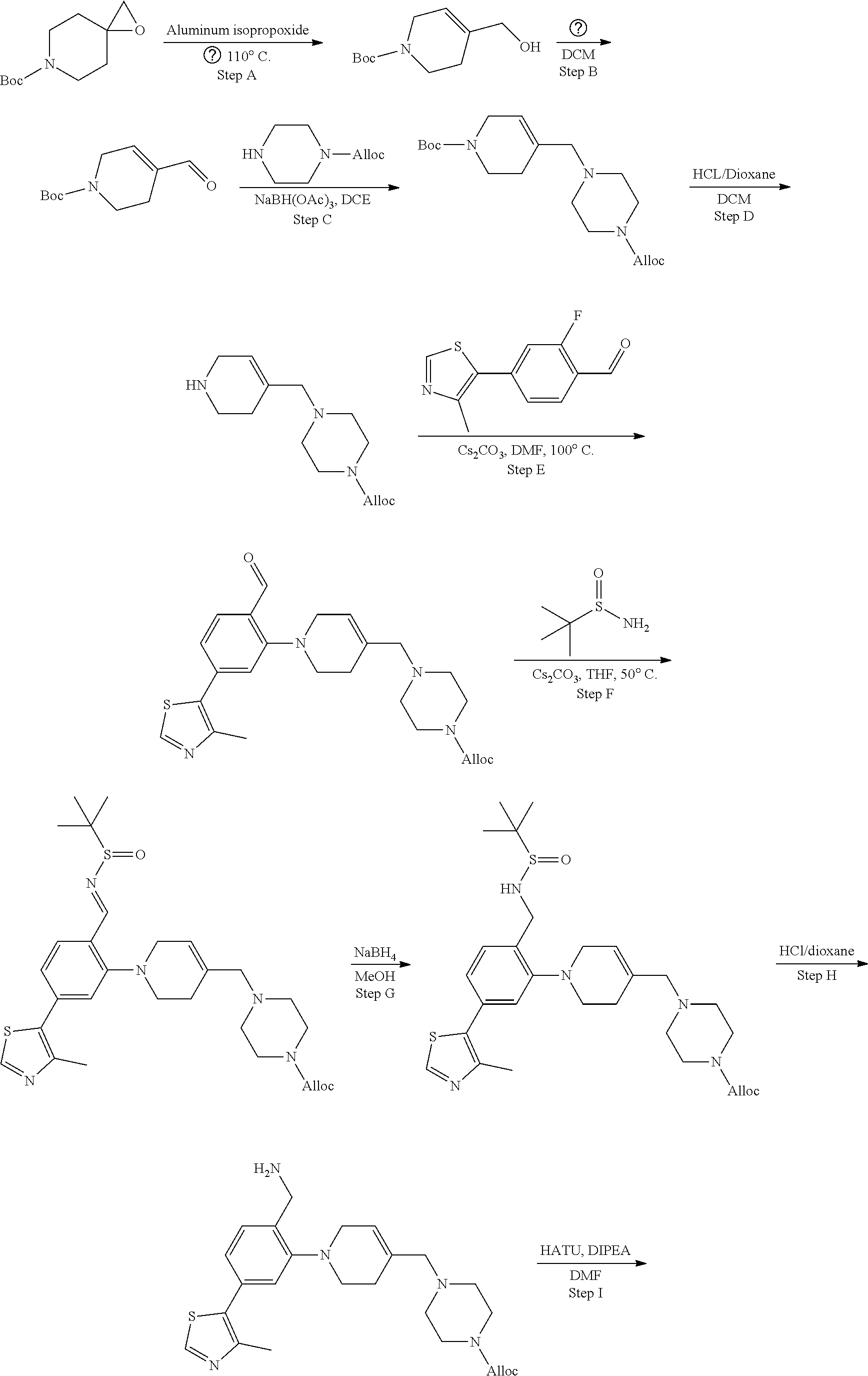

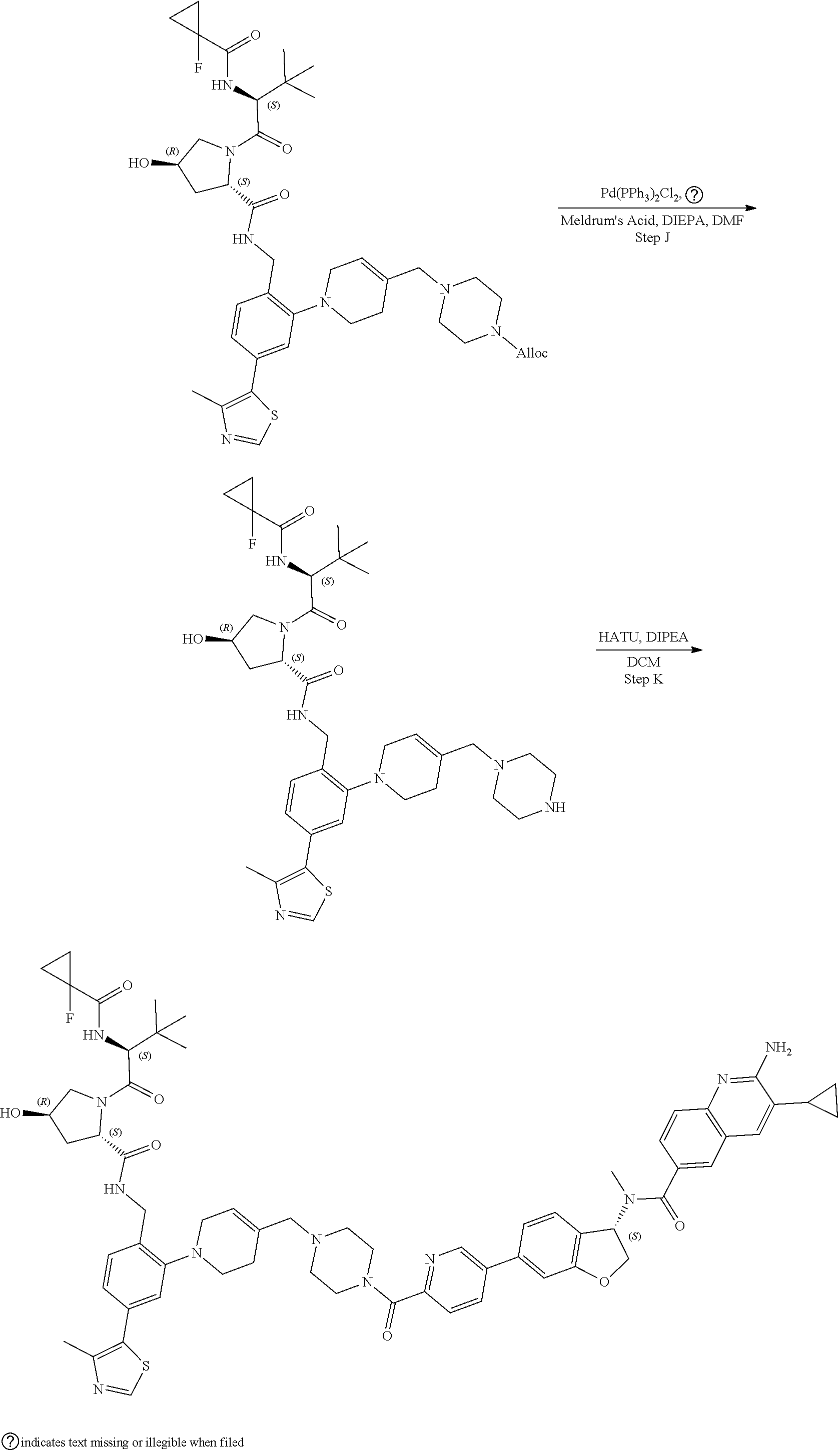

Step A: tert-butyl 4-(hydroxymethyl)-3,6-dihydropyridine-1 (2H)-carboxylate

A mixture of tert-butyl 1-oxa-6-azaspiro[2.5]octane-6-carboxylate (8 g, 37.5 mmol, 1.0 eq.) and Aluminum isopropoxide (11.5 g, 56.3 mmol, 1.5 eq.) in Toluene (80 mL) was stirred at 110° C. for 16 hours under nitrogen. After cooling to room temperature, the resulting solution was diluted with 200 ml of water and extracted with EtOAc (100 mL×3). The combined organic layers were washed with brine, dried over Na₂SO₄ and concentrated under reduced pressure. The crude product was purified by flash column chromatography on silica gel (100-200 mesh silica gel, 0~50% EtOAc in PE) to afford tert-butyl 4-(hydroxymethyl)-3,6-dihydropyridine-1 (2H)-carboxylate (5 g, 62% yield) as a yellow solid. LC-MS (ESI): mass calcd. For C₁₁H₁₉NO₃, 213.14; m/z found, 214 [M+H]⁺.

Step B: tert-butyl 4-formyl-3,6-dihydropyridine-1 (2H)-carboxylate

A mixture of tert-butyl 4-(hydroxymethyl)-3,6-dihydropyridine-1 (2H)-carboxylate (2.8 g, 13 mmol, 1.0 eq.) and manganese (IV) oxide (11 g, 130 mmol, 10.0 eq.) in DCM (5 mL) was stirred at room temperature for 16 hours under nitrogen. After cooling to room temperature, the resulting solution was diluted with 100 mL of water and extracted with EtOAc (100 mL×3). The combined organic layers were washed with brine, dried over Na₂SO₄ and concentrated under reduced pressure. The crude product was purified by flash column chromatography on silica gel (100-200 mesh silica gel, 0~15% EtOAc in PE) to afford tert-butyl 4-formyl-3,6-dihydropyridine-1 (2H)-carboxylate (2 g, 70% yield) as a yellow solid. LC-MS (ESI): mass calcd. For C₁₁H₁₇NO₃, 211.12; m/z found, 212 [M+H]⁺.

Step C: allyl 4-((1-(tert-butoxycarbonyl)-1,2,3,6-tetrahydropyridin-4-yl)methyl)piperazine-1-carboxylate

A mixture of tert-butyl 4-formyl-3,6-dihydropyridine-1 (2H)-carboxylate (360 mg, 1.7 mmol, 1.0 eq.) in DCM (5 mL) was added TEA (0.46 mL, 3.4 mmol, 2.0 eq.). The mixture was stirred at room temperature for 30 minutes. Then the allyl piperazine-1-carboxylate (348 mg, 2.04 mmol, 1.2 eq.) and AcOH (204 mg, 3.4 mmol, 2.0 eq.) was added. After 30 minutes NaBH (OAc) 3 (1.1 g, 5.2 mmol, 3.0 eq.) was added. The mixture was stirred at room temperature for 3 hours under nitrogen. After cooling to room temperature, the resulting solution was diluted with 50 mL of water and extracted with EtOAc (50 mL×3). The combined organic layers were washed with brine, dried over Na₂SO₄ and concentrated under reduced pressure. The crude product was purified by flash column chromatography on silica gel (100-200 mesh silica gel, 0~30% EtOAc in PE) to afford allyl 4-((1-(tert-butoxycarbonyl)-1,2,3,6-tetrahydropyridin-4-yl)methyl)piperazine-1-carboxylate (250 mg, 40% yield) as a yellow oil. LC-MS (ESI): mass calcd. For C₁₉H₃₁N₃O₄, 365.23; m/z found, 366 [M+H]⁺.

Step D: allyl 4-((1,2,3,6-tetrahydropyridin-4-yl)methyl)piperazine-1-carboxylate

To a solution of allyl 4-((1-(tert-butoxycarbonyl)-1,2,3,6-tetrahydropyridin-4-yl)methyl)piperazine-1-carboxylate (500 mg, 1.4 mmol, 1.0 eq.) in DCM (3 mL) was added HCl/Dioxane (4M, 1 mL) at room temperature. The mixture was stirred at room temperature for 2 hours under nitrogen. The resulting solution was removed under reduced pressure to afford allyl 4-((1,2,3,6-tetrahydropyridin-4-yl)methyl)piperazine-1-carboxylate (350 mg, 96% crude) as a yellow solid. LC-MS (ESI): mass calcd. For C₁₄H₂₃N₃O₂, 265.18; m/z found, 266 [M+H]⁺.

Steps E-K: 2-amino-3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-1,2,3,6-tetrahydropyridin-4-yl)methyl)piperazine-1-carbonyl)pyridin-3-yl)-2,3-dihydrobenzofuran-3-yl)-N-methylquinoline-6-carboxamide

Example A229 was synthesized following the same procedures as described in Example A107 (3.1 mg, 2% yield over 7 steps) as a white solid. LC-MS (ESI): mass calcd. for C₆₄H₇₂FN₁₁O₇S, 1157.53; m/z found, 1158.6 [M+H]⁺. ¹H NMR (400 MHz, DMSO-d₆) δ 9.73 (s, 1H), 8.98 (s, 1H), 8.93 (s, 1H), 8.65 (s, 1H), 8.53 (s, 1H), 8.28 (d, J=9.3 Hz, 1H), 8.18 (s, 1H), 8.03 (s, 1H), 7.84 (d, J=9.0 Hz, 1H), 7.76 (s, 2H), 7.55 (d, J=7.9 Hz, 1H), 7.46 (d, J=8.0 Hz, 1H), 7.41 (s, 1H), 7.35 (s, 1H), 7.27 (d, J=9.1 Hz, 1H), 7.18-7.13 (m, 2H), 6,14-6.08 (m, 1H), 4.87-4.58 (m, 4H), 4.49 (t, J=8.2 Hz, 1H), 4.41-4.21 (m, 4H), 3.94-3.68 (m, 2H), 3.65-3.54 (m, 5H), 3.11-3.00 (m, 4H), 2.74-2.57 (m, 5H), 2.46 (s, 3H), 2.35-2.29 (m, 3H), 2.10-2.07 (m, 1H), 1.92-1.87 (m, 2H), 1.41-1.37 (m, 1H), 1.36-1.32 (m, 1H), 1.23-1.20 (m, 2H), 1.09-1.06 (m, 2H), 0.96 (s, 9H), 0.82-0.79 (m, 2H).

### Example A230. 2-amino-3-cyano-N-((3S)-6-(6-((2-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclopentyl)carbamoyl) pyridin-3-yl)-2,3-dihydrobenzofuran-3-yl)-N-methylquinoline-6-carboxamide

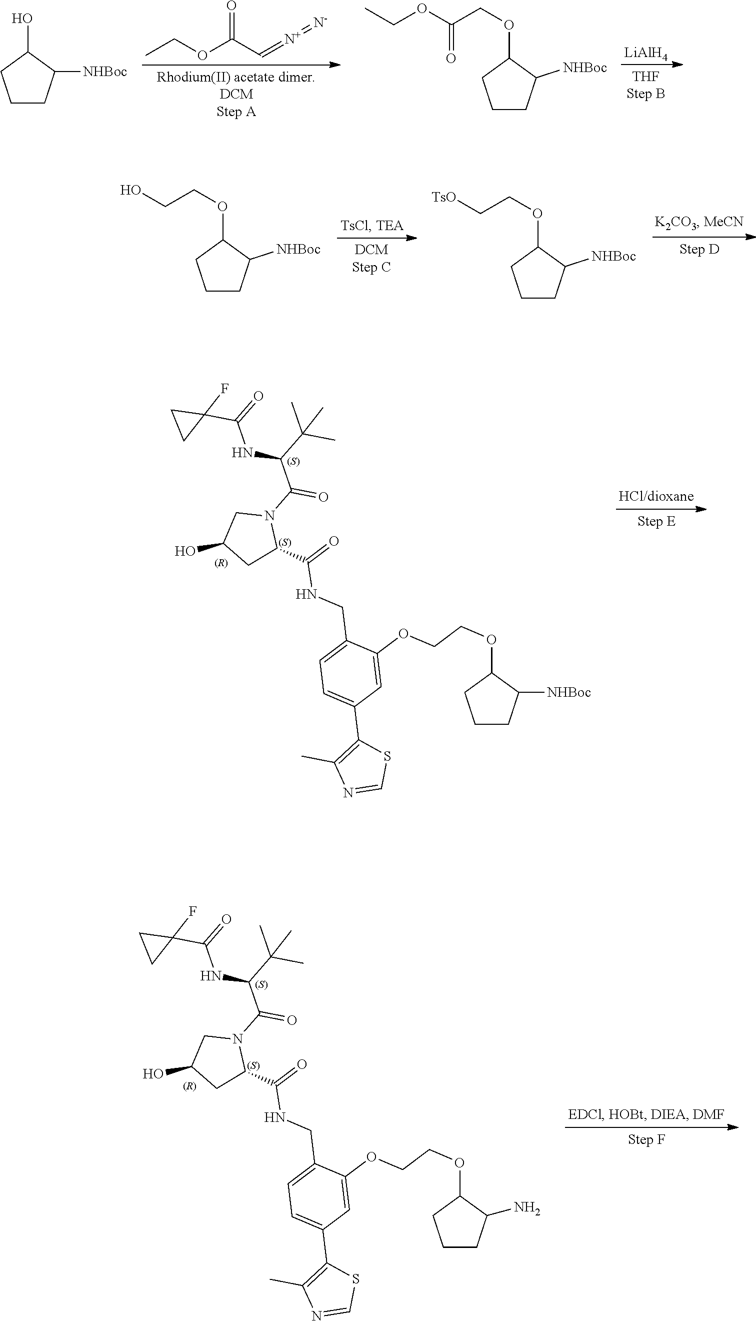

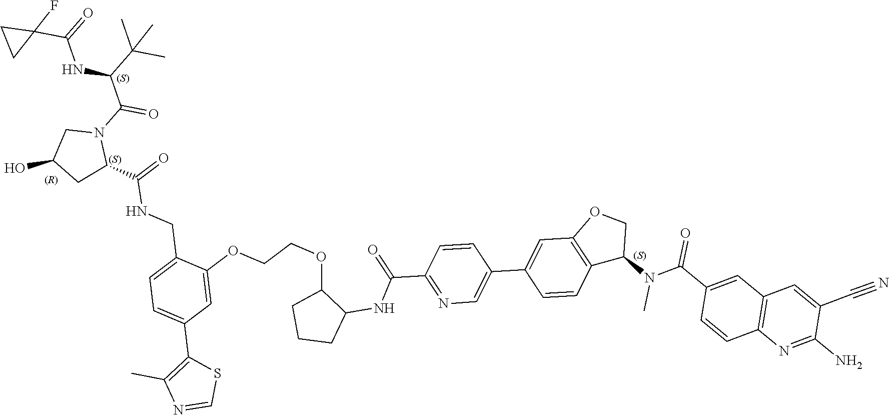

Example A230 was synthesized following the same procedures as described in Example A220 (14.6 mg, 1.2% yield over 6 steps) as a white solid. LC-MS (ESI): mass calcd. for C₅₉H₆₃FN₁₀O₇S, 1107.27; m/z found, 1108.5 [M+H]⁺. ¹H NMR (400 MHz, DMSO-d₆) δ 8.96 (s, 1H), 8.89 (s, 1H), 8.85-8.73 (m, 2H), 8.47 (s, 1H), 8.25 (dd, J=8.1, 2.0 Hz, 1H), 8.07 (d, J=8.1 Hz, 1H), 7.97 (s, 1H), 7.84-7.79 (m, 1H), 7.60-7.54 (m, 2H), 7.42 (s, 1H), 7.38 (d, J=7.7 Hz, 2H), 7.33 (s, 1H), 7.28 (dd, J=9.2, 2.5 Hz, 1H), 7.00 (s, 1H), 6.93 (dd, J=7.7, 3.7 Hz, 1H), 4.69 (d, J=5.2 Hz, 1H), 4.59 (d, J=8.9 Hz, 2H), 4.54-4.48 (m, 1H), 4.45 (d, J=8.3 Hz, 1H), 4.33 (s, 1H), 4.31-4.24 (m, 2H), 4.22 (d, J=4.9 Hz, 1H), 4.16 (s, 2H), 4.04-4.01 (m, 1H), 3.91-3.86 (m, 1H), 3.82-3.77 (m, 1H), 3.66-3.57 (m, 2H), 2.67 (s, 3H), 2.43 (d, J=1.1 Hz, 3H), 2.12-1.99 (m, 2H), 1.99-1.88 (m, 2H), 1.75-1.65 (m, 2H), 1.65-1.58 (m, 2H), 1.40-1.30 (m, 2H), 1.23-1.17 (m, 2H), 0.94 (s, 9H).

### Example A231. 2-amino-3-cyano-N-((S)-6-(6-((5-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)-2-methylpentan-2-yl)carbamoyl)pyridin-3-yl)-2,3-dihydrobenzofuran-3-yl)-N-methylquinoline-6-carboxamide

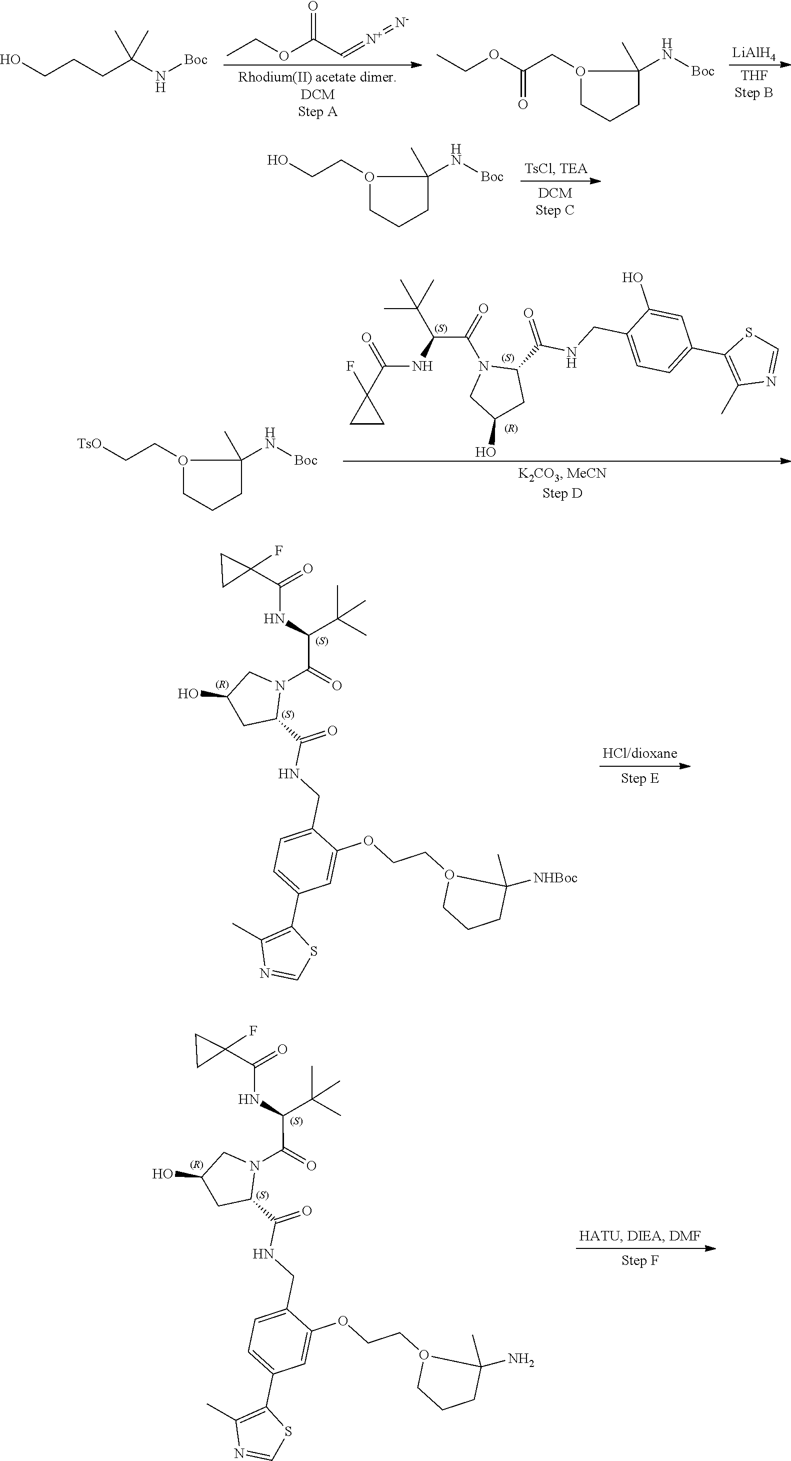

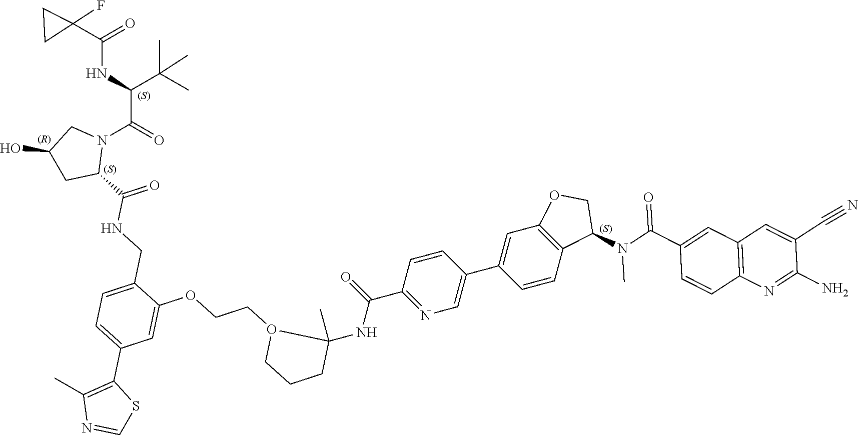

Example A231 was synthesized following the same procedures as described in Example A220 (9.7 mg, 7% yield over 6 steps) as a white solid. LC-MS (ESI): mass calcd. for C₆₀H₆₇FN₁₀O₉S, 1122.48; m/z found, 1123 [M+H]⁺. ¹H NMR (400 MHz, DMSO-d₆) δ 8.97 (s, 1H), 8.91 (s, 1H), 8.83 (s, 1H), 8.48 (t, J=6.0 Hz, 1H), 8.27 (dd, J=8.1, 2.2 Hz, 1H), 8.06 (d, J=8.2 Hz, 1H), 8.00-7.96 (m, 2H), 7.84-7.80 (m, 1H), 7.64-7.58 (m, 1H), 7.55 (d, J=7.8 Hz, 1H), 7.39 (t, J=8.8 Hz, 3H), 7.32 (s, 1H), 7.29-7.25 (m, 1H), 7.04 (s, 1H), 6.95 (d, J=7.8 Hz, 1H), 4.71-4.67 (m, 2H), 4.58 (d, J=9.3 Hz, 2H), 4.51 (t, J=8.2 Hz, 3H), 4.34-4.33 (m, 1H), 4.29 (d, J=6.2 Hz, 1H), 4.22-4.16 (m, 4H), 3.75-3.73 (m, 2H), 3.64-3.58 (m, 2H), 3.50 (t, J=6.4 Hz, 2H), 2.67 (s, 3H), 2.44 (s, 3H), 2.10-2.04 (m, 1H), 1.93-1.87 (m, 1H), 1.84-1.79 (m, 2H), 1.56-1.50 (m, 2H), 1.38 (s, 6H), 1.36-1.29 (m, 2H), 1.23-1.18 (m, 2H), 0.94 (s, 9H).

### Example A233. 2-amino-3-cyano-N-((S)-6-(6-(((1S,2R)-2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)

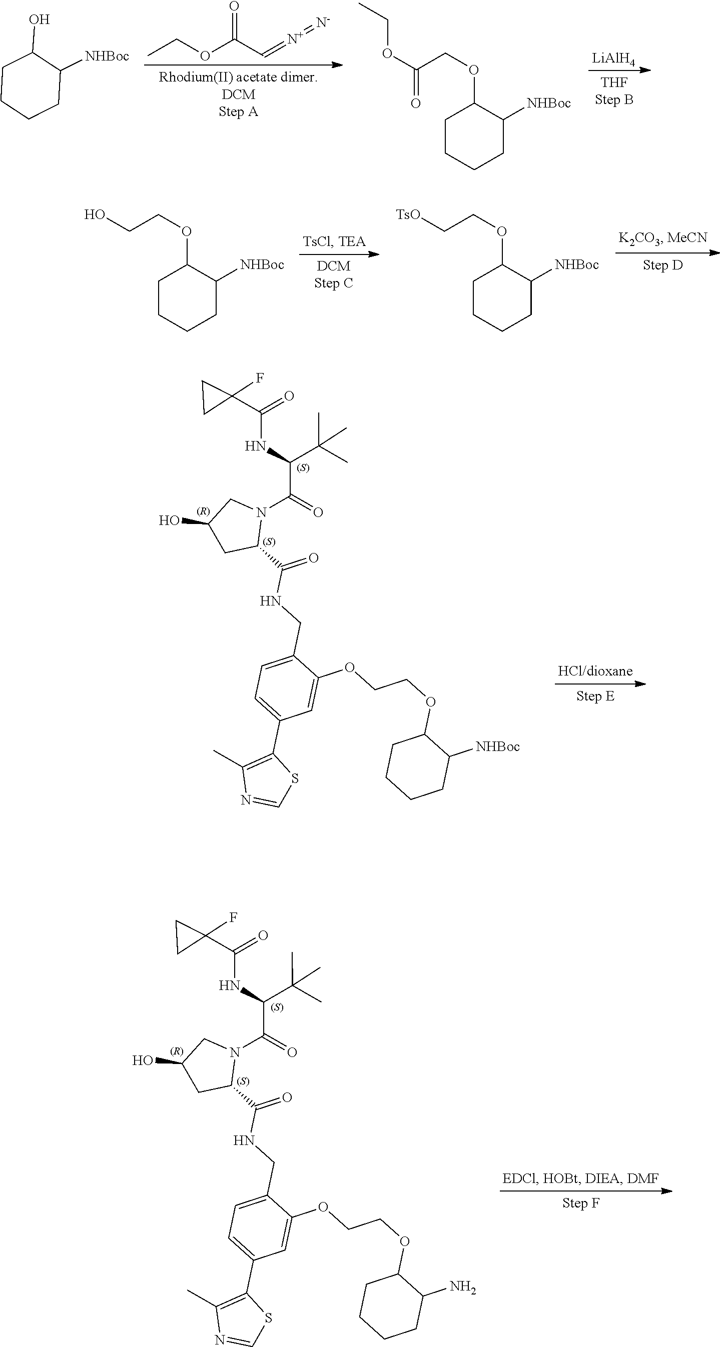

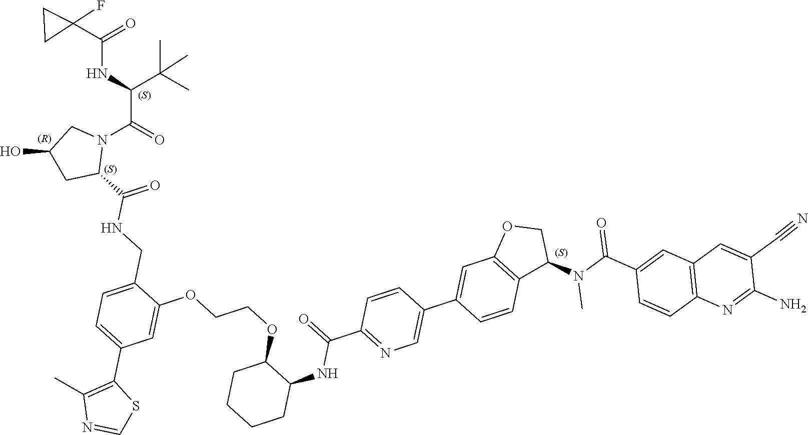

Example 251 was synthesized following the same procedures as described in Example A220 (6.9 mg, 21% yield over 6 steps) as a white solid. LC-MS (ESI): mass calcd. for C₆₀H₆₈FN₁₀O₉S, 1120.46/z found, 1122 [M+H]⁺. ¹H NMR (400 MHz, DMSO) δ 8.91 (s, 1H), 8.82 (s, 1H), 8.66-8.59 (m, 1H), 8.55 (s, 1H), 8.41 (t, J=6.0 Hz, 1H), 8.18-8.13 (m, 1H), 8.08-8.01 (m, 1H), 7.97 (s, 1H), 7.81 (dd, J=8.7, 1.8 Hz, 1H), 7.64-7.56 (m, 1H), 7.54-7.42 (m, 2H), 7.33-7.23 (m, 4H), 6.89-6.78 (m, 2H), 4.70-4.66 (m, 2H), 4.62-4.58 (m, 1H), 4.53 (t, J=8.2 Hz, 1H), 4.35 (s, 1H), 4.24-4.14 (m, 2H), 4.11-4.01 (m, 3H), 3.90-3.80 (m, 4H), 3.55-3.35 (m, 2H), 2.67 (s, 3H), 2.36 (s, 3H), 2.15-2.03 (m, 2H), 1.96-1.85 (m, 2H), 1.74-1.61 (m, 2H), 1.52-1.42 (m, 1H), 1.40-1.36 (m, 1H), 1.36-1.31 (m, 1H), 1.29-1.19 (m, 5H), 0.94 (s, 9H).

### Example A234. 2-amino-3-cyano-N-((S)-6-(6-((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)benzyl)carbamoyl)pyridin-3-yl)-2,3-dihydrobenzofuran-3-yl)-N-methylquinoline-6-carboxamide

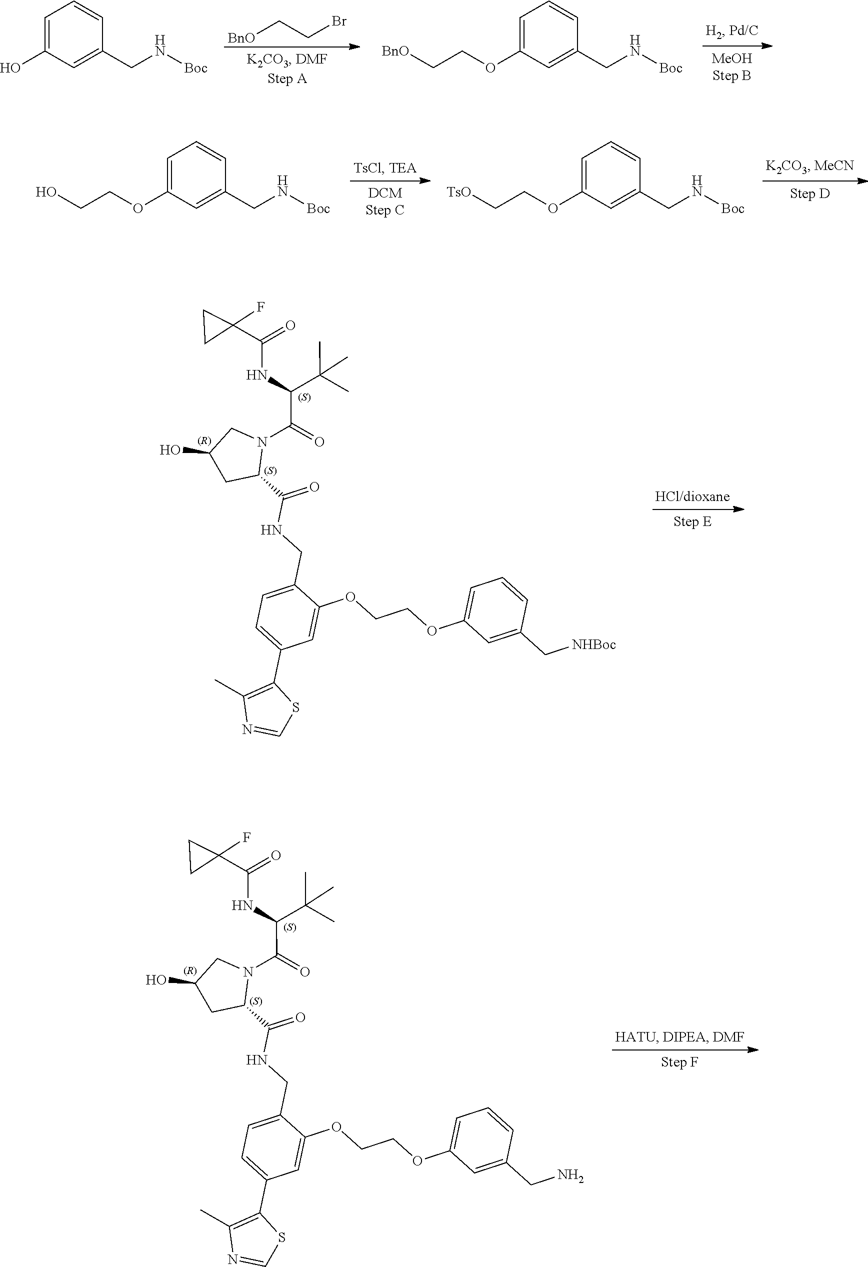

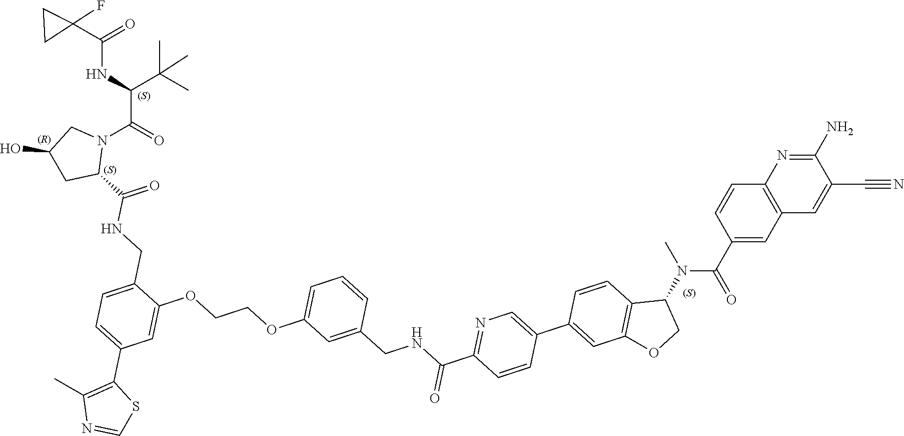

Step A: tert-butyl (3-(2-(benzyloxy)ethoxy)benzyl)carbamate

To a solution of tert-butyl (3-hydroxybenzyl)carbamate (5 g, 22.4 mmol, 1.0 eq.), ((2-bromoethoxy)methyl) benzene (7.19 g, 33.6 mmol, 1.5 eq.) in DMF (50.0 mL) was added K₂CO₃ (9.28 g, 67.3 mmol, 3.0 eq.). The mixture was stirred at room temperature for 3 hours. The resulting solution was diluted with 100 mL of water and extracted with EtOAc (80 mL×3). The combined organic layers were washed with brine (80 mL), dried over Na₂SO₄ and concentrated under reduced pressure. The residue was purified by flash column chromatography on silica gel (100-200 mesh silica gel, 0-20% EtOAc in PE) to give tert-butyl (3-(2-(benzyloxy)ethoxy)benzyl)carbamate (4.5 g, 56% yield) as a yellow oil. LC-MS (ESI): mass calcd. for C37H53FN408, 700.38; m/z found, 701.6 [M+H]⁺.

Step B-F: 2-amino-3-cyano-N-((S)-6-(6-((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)benzyl)carbamoyl)pyridin-3-yl)-2,3-dihydrobenzofuran-3-yl)-N-methylquinoline-6-carboxamide

Example A234 was synthesized following the same procedures as described in Example A29 (5.2 mg, 4% yield over 6 steps) as a white solid. LC-MS (ESI): mass calcd. for C₆₁H₆₁FN₁₀O₉S, 1128.43; m/z found, 1129.8 [M+H]⁺. ¹HNMR (400 MHz, DMSO-d₆) δ 9.34 (t, J=6.5 Hz, 1H), 8.98 (s, 1H), 8.94 (d, J=1.1 Hz, 1H), 8.84 (s, 1H), 8.48 (t, J=5.9 Hz, 1H), 8.28 (dd, J=8.2, 2.1 Hz, 1H), 8.10 (d, J=8.2 Hz, 1H), 7.98 (s, 1H), 7.83 (dd, J=8.8, 1.8 Hz, 1H), 7.67-7.46 (m, 4H), 7.42-7.38 (m, 2H), 7.35 (s, 1H), 7.29-7.22 (m, 2H), 7.09 (d, J=0.7 Hz, 1H), 6.97 (d, J=6.7 Hz, 2H), 6.93 (d, J=7.7 Hz, 1H), 6.89 (dd, J=8.2, 2.2 Hz, 1H), 4.71-4.67 (m, 1H), 4.58 (d, J=9.2 Hz, 1H), 4.52-4.48 (m, 3H), 4.42-4.38 (m, 2H), 4.35-4.31 (m, 3H), 4.27 (d, J=6.1 Hz, 1H), 4.21-4.14 (m, 1H), 3.64-3.57 (m, 4H), 2.67 (s, 3H), 2.45 (s, 3H), 2.09-2.04 (m, 1H), 1.93-1.87 (m, 1H), 1.40-1.31 (m, 2H), 1.23-1.19 (m, 2H), 0.94 (s, 9H).

### Example A235. 2-amino-3-cyano-N-((3S)-6-(6-(3-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-3,8-diazabicyclo[3.2.1]octane-8-carbonyl)pyridin-3-yl)-2,3-dihydrobenzofuran-3-yl)-N-methylquinoline-6-carboxamide

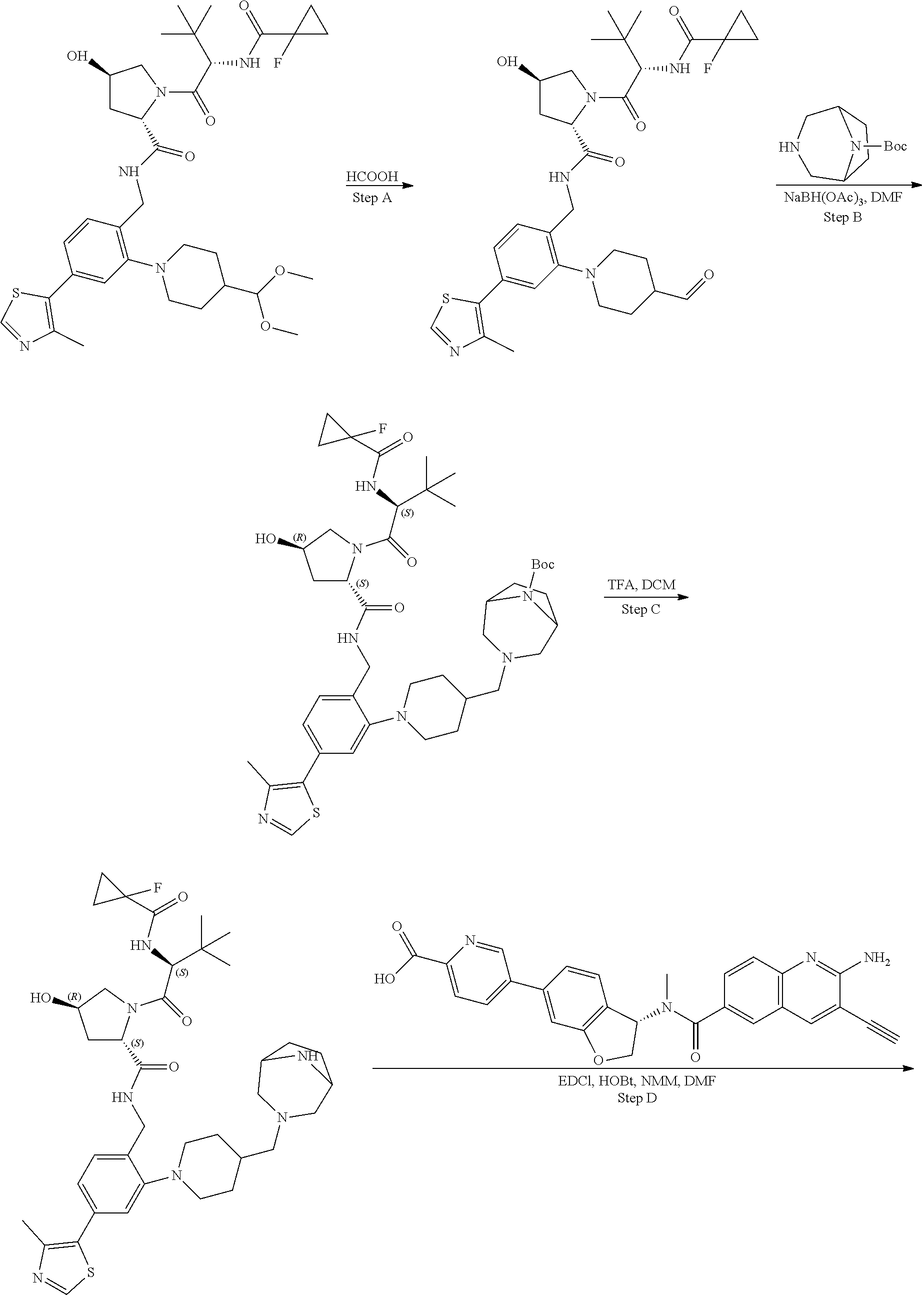

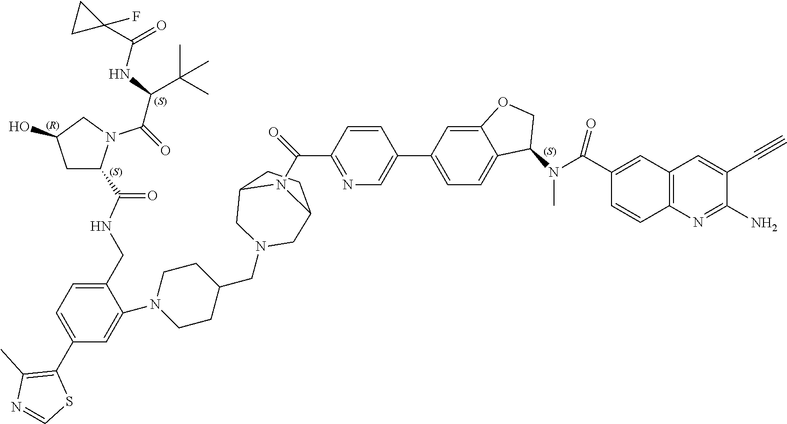

Example A235 was synthesized following the same procedures as described in Example A107 (2.0 mg, 2% yield over 4 steps) as a white solid. LC-MS (ESI): mass calcd. for C₆₄H₇₁FN₁₂O₇S, 1170.52; m/z found, 1172.1 [M+H]⁺. ¹H NMR (400 MHz, DMSO-d₆) δ 8.98 (s, 1H), 8.93 (s, 1H), 8.77 (s, 1H), 8.49 (s, 1H), 8.23 (d, J=8.8 Hz, 1H), 7.95 (s, 1H), 7.80 (t, J=8.5 Hz, 2H), 7.57-7.54 (m, 2H), 7.41 (t, J=8.5 Hz, 2H), 7.34 (s, 1H), 7.28 (d, J=7.9 Hz, 1H), 7.19 (s, 2H), 7.10 (d, J=8.8 Hz, 2H), 5.17 (s, 1H), 4.77-4.64 (m, 4H), 4.60 (d, J=9.0 Hz, 1H), 4.48 (t, J=8.0 Hz, 1H), 4.42-4.27 (m, 3H), 3.68-3.59 (m, 2H), 3.07 (d, J=12.1 Hz, 2H), 2.82-2.61 (m, 10H), 2.46 (s, 3H), 2.32-2.23 (m, 4H), 2.11-2.06 (m, 1H), 1.90-1.81 (m, 5H), 1.64 (s, 1H), 1.41-1.29 (m, 4H), 1.21 (d, J=7.7 Hz, 2H), 0.98 (s, 9H).

### Example A236. 2-amino-3-cyano-N-(S)-6-(6-(((1S,2R)-2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)

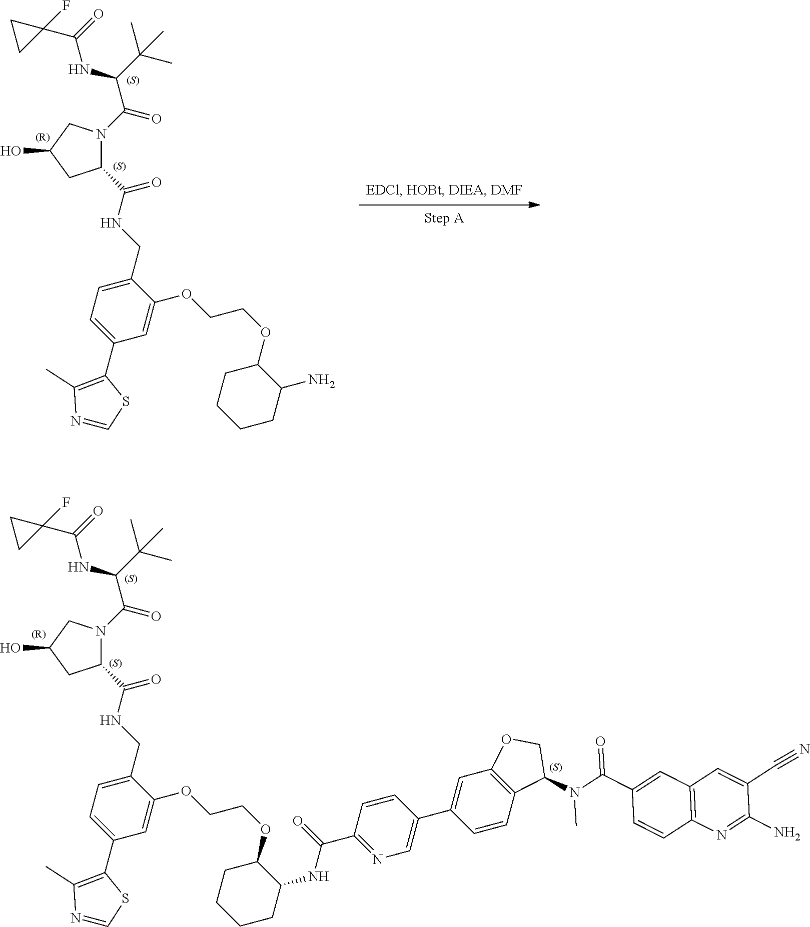

Example A254 was synthesized following the same procedures as described in Example A107 (6.9 mg, 21% yield) as a white solid. LC-MS (ESI): mass calcd, for C₆₀H₆₅FN10O9S, 1120.46 m/z found, 1122 [M+H]⁺. ¹H NMR (400 MHz, DMSO-d₆) δ 8.91 (s, 1H), 8.84 (s, 1H), 8.66-8.55 (m, 2H), 8.42 (t, J=5.8 Hz, 1H), 8.16-8.10 (m, 1H), 8.07-8.01 (m, 1H), 7.98 (s, 1H), 7.83 (dd, J=8.7, 1.7 Hz, 1H), 7.64-7.58 (m, 1H), 7.50 (d, J=7.6 Hz, 2H), 7.34-7.20 (m, 4H), 6.91-6.83 (m, 2H), 4.81-4.66 (m, 2H), 4.59 (d, J=9.2 Hz, 1H), 4.51 (t, J=8.2 Hz, 1H), 4.33 (s, 1H), 4.23-4.14 (m, 2H), 4.11-4.00 (m, 3H), 3.92-3.75 (m, 4H), 3.50-3.41 (m, 2H), 2.66 (s, 3H), 2.37 (s, 3H), 2.15-2.02 (m, 2H), 1.94-1.85 (m, 2H), 1.74-1.60 (m, 2H), 1.49-1.42 (m, 1H), 1.40-1.37 (m, 1H), 1.36-1.31 (m, 1H), 1.28-1.18 (m, 5H), 0.95 (s, 9H).

### Example A237. 2-amino-3-cyano-N-((3S)-6-(6-(5-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-2,5-diazabicyclo[2.2.1]heptane-2-carbonyl)pyridin-3-yl)-2,3-dihydrobenzofuran-3-yl)-N-methylquinoline-6-carboxamide

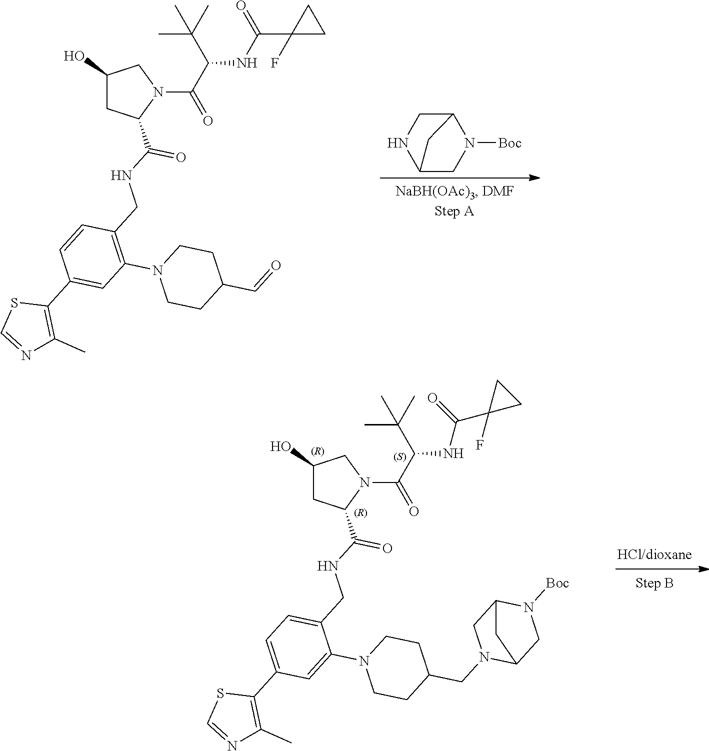

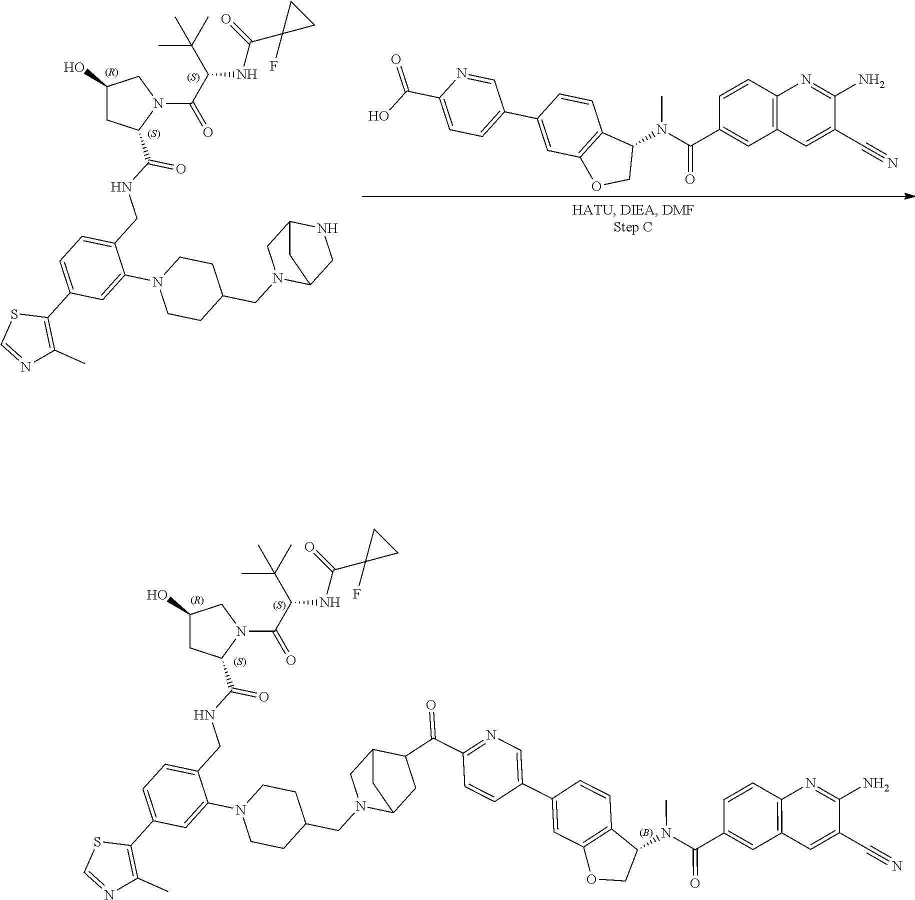

Example A237 was synthesized following the same procedures as described in Example A107 (9.0 mg, 4% yield over 3 steps) as a white solid. LC-MS (ESI): mass calcd. for C₆₃H₆₉FN₁₂O₇S, 1156.51; m/z found, 1157.9 [M+H]⁺. ¹HNMR (400 MHz, DMSO-d₆) δ 9.00-8.94 (m, 2H), 8.80 (s, 1H), 8.51 (d, J=5.2 Hz, 1H), 8.30 (d, J=9.6 Hz, 1H), 8.05-7.92 (m, 2H), 7.80 (d, J=8.7 Hz, 1H), 7.60-7.54 (m, 2H), 7.45-7.33 (m, 5H), 7.27 (d, J=8.9 Hz, 1H), 7.13 (d, J=6.9 Hz, 2H), 5.40-4.92 (m, 2H), 4.85-4.65 (m, 2H), 4.60 (dd, J=8.5, 1.8 Hz, 1H), 4.51 -4.45 (m, 2H), 4.38-4.32 (m, 3H), 4.20 (d, J=11.8 Hz, 1H), 4.09-4.03 (m, 1H), 3.99-3.95 (m, 1H), 3.93-3.86 (m, 1H), 3.17-3.01 (m, 5H), 2.72-2.65 (m, 5H), 2.45 (d, J=4.1 Hz, 3H), 2.34-2.32 (m, 1H), 2.21-2.16 (m, 1H), 2.10-2.07 (m, 1H), 1.95-1.85 (m, 4H), 1.45-1.32 (m, 4H), 1.21 (d, J=7.7 Hz, 2H), 0.97 (s, 9H).

### Example A239. 2-amino-3-cyano-N-((3S)-6-(6-(((1-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl) azetidin-2-yl)methyl) carbamoyl)pyridin-3-yl)-2,3-dihydrobenzofuran-3-yl)-N-methylquinoline-6-carboxamide

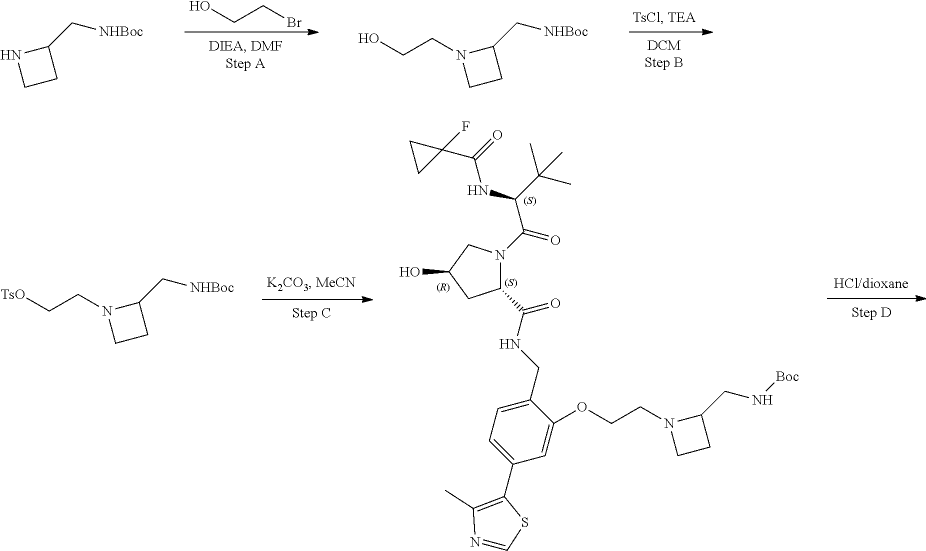

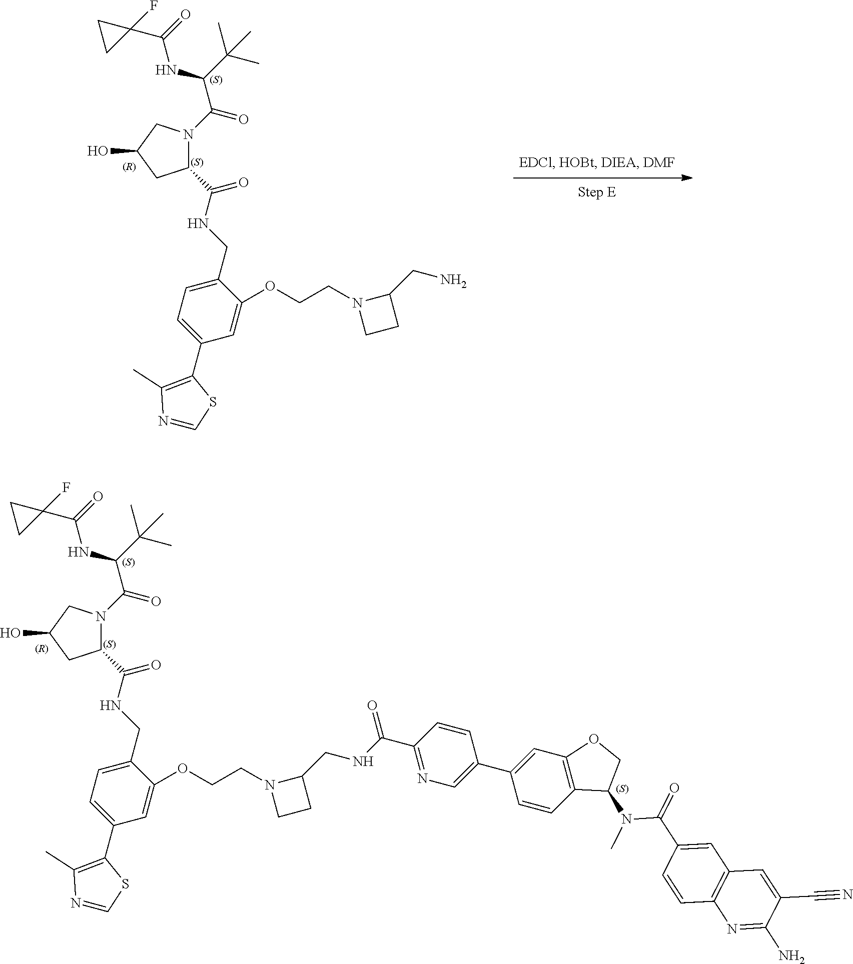

Step A: tert-butyl ((1-(2-hydroxyethyl) azetidin-2-yl)methyl)carbamate

To a mixture of tert-butyl (azetidin-2-ylmethyl)carbamate (100 mg, 537 μmol, 1.0 eq.) in DMF (5 mL) was added N-ethyl-N-isopropylpropan-2-amine (139 mg, 1.1 mmol, 2.0 eq.) and 2-bromoethan-1-ol (80.5 mg, 644 μmol, 1.2 eq.). The reaction mixture was stirred at 40° C. for 2 hours. The reaction mixture was poured into water (30 mL) and then extracted with EtOAc (30 mL×2). The combined organic layers were washed with brine, dried over anhydrous Na₂SO₄ and concentrated to afford crude tert-butyl ((1-(2-hydroxyethyl) azetidin-2-yl)methyl)carbamate (80 mg, 65% yield) as a yellow oil. LC-MS (ESI): mass calcd. For C1H22N203, 230.16; m/z found, 231.2 [M+H]⁺.

Step B-E: 2-amino-3-cyano-N-(3S)-6-(6-(((1-(2-(2-((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl) azetidin-2-yl)methyl)carbamoyl)pyridin-3-yl)-2,3-dihydrobenzofuran-3-yl)-N-methylquinoline-6-carboxamide

Example A239 was synthesized following the same procedures as described in Example A23 (1.1 mg, 1% yield over 4 steps) as a white solid. LC-MS (ESI): mass calcd. for C₅₈H₆₂FN110&S, 1091.45; m/z found, 1092.1 [M+H]⁺. ¹H NMR (400 MHz, DMSO-d₆) δ 8.95 (d, J=3.7 Hz, 1H), 8.88 (s, 1H), 8.77 (s, 1H), 8.59-8.38 (m, 1H), 8.30-8.13 (m, 1H), 8.04 (d, J=7.9 Hz, 1H), 7.94 (s, 1H), 7.78 (d, J=8.3 Hz, 1H), 7.63-7.48 (m, 2H), 7.46-7.14 (m, 6H), 7.03-6.89 (m, 2H), 4.80-4.64 (m, 2H), 4.64-4.47 (m, 2H), 4.41-4.13 (m, 4H), 3.64-3.57 (m, 2H), 3.23-3.04 (m, 3H), 2.67 (s, 3H), 2.58-2.53 (m, 2H), 2.42 (s, 3H), 2.35-2.29 (m, 2H), 2.12-1.84 (m, 5H), 1.48-1.12 (m, 12H).

### Example A240. 2-amino-3-cyano-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)-4-methylcyclohexyl) carbamoyl)pyridin-3-yl)-2,3-dihydrobenzofuran-3-yl)-N-methylquinoline-6-carboxamide

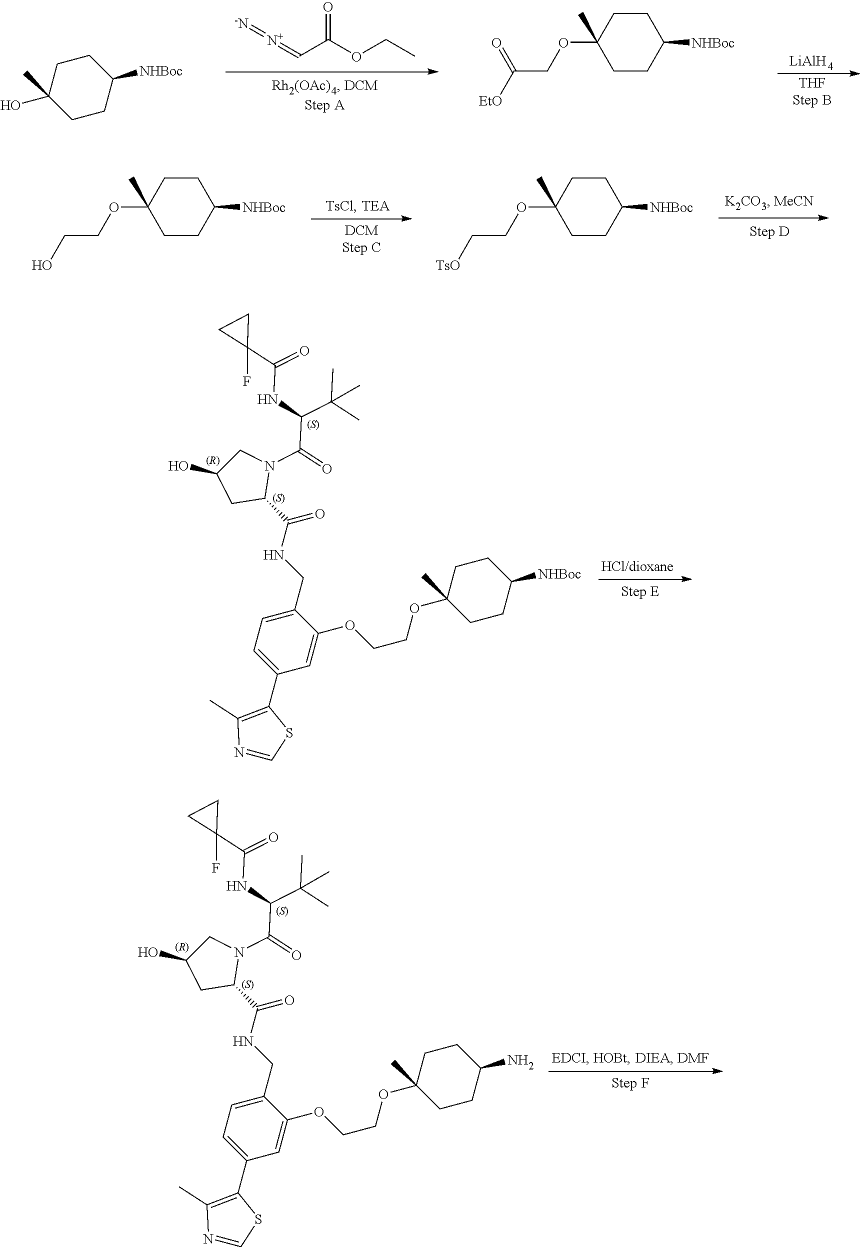

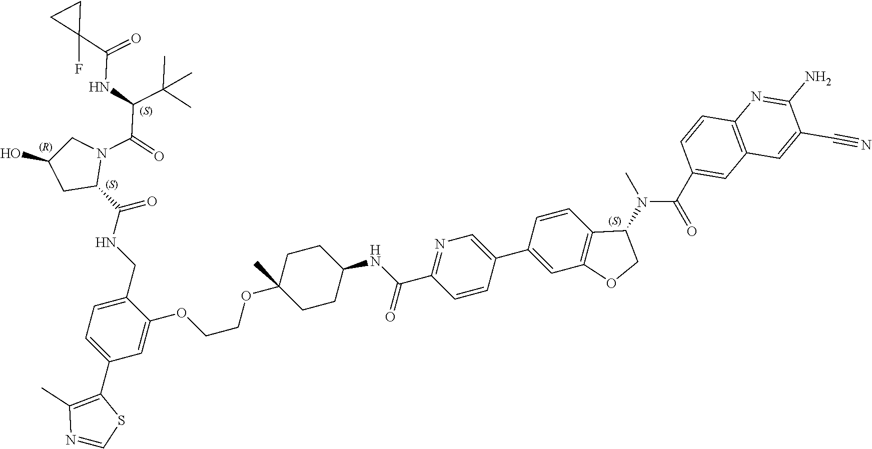

Example A240 was synthesized following the same procedures as described in Example A220 (8.3 mg, 2% yield over 6 steps) as a white solid. LC-MS (ESI): mass calcd. for C₆₁H₆₇FN₁₀O₉S, 1134.48; m/z found, 1135.6 [M+H]⁺. ¹H NMR (400 MHz, DMSO-d₆) δ 9.04-8.87 (m, 1H), 8.81 (s, 1H), 8.52 (d, J=8.1 Hz, 2H), 8.28 (d, J=8.5 Hz, 1H), 8.08 (d, J=8.3 Hz, 1H), 7.97 (s, 1H), 7.81 (s, 1H), 7.56 (d, J=8.3 Hz, 2H), 7.47-7.25 (m, 5H), 7.06 (s, 1H), 6.97 (d, J=8.1 Hz, 1H), 4.71-4.67 (m, 1H), 4.62-4.57 (m, 1H), 4.55-4.50 (m, 1H), 4.39-4.26 (m, 4H), 4.25-4.20 (m, 1H), 4.15 (s, 3H), 3.78-3.68 (m, 1H), 3.70-3.53 (m, 2H), 2.65 (s, 3H), 2.47 (s, 3H), 2.36-2.26 (m, 1H), 2.15-2.05 (m, 1H), 1.99-1.89 (m, 1H), 1.85-1.74 (m, 2H), 1.73 -1.55 (m, 6H), 1.45-1.31 (m, 2H), 1.30-1.18 (m, 6H), 0.96 (s, 9H).

### Example A241. 2-amino-3-cyano-N-((S)-6-(6-(4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-1-yl)methyl)piperidine-1-carbonyl)pyridin-3-yl)-2,3-dihydrobenzofuran-3-yl)-N-methylquinoline-6-carboxamide

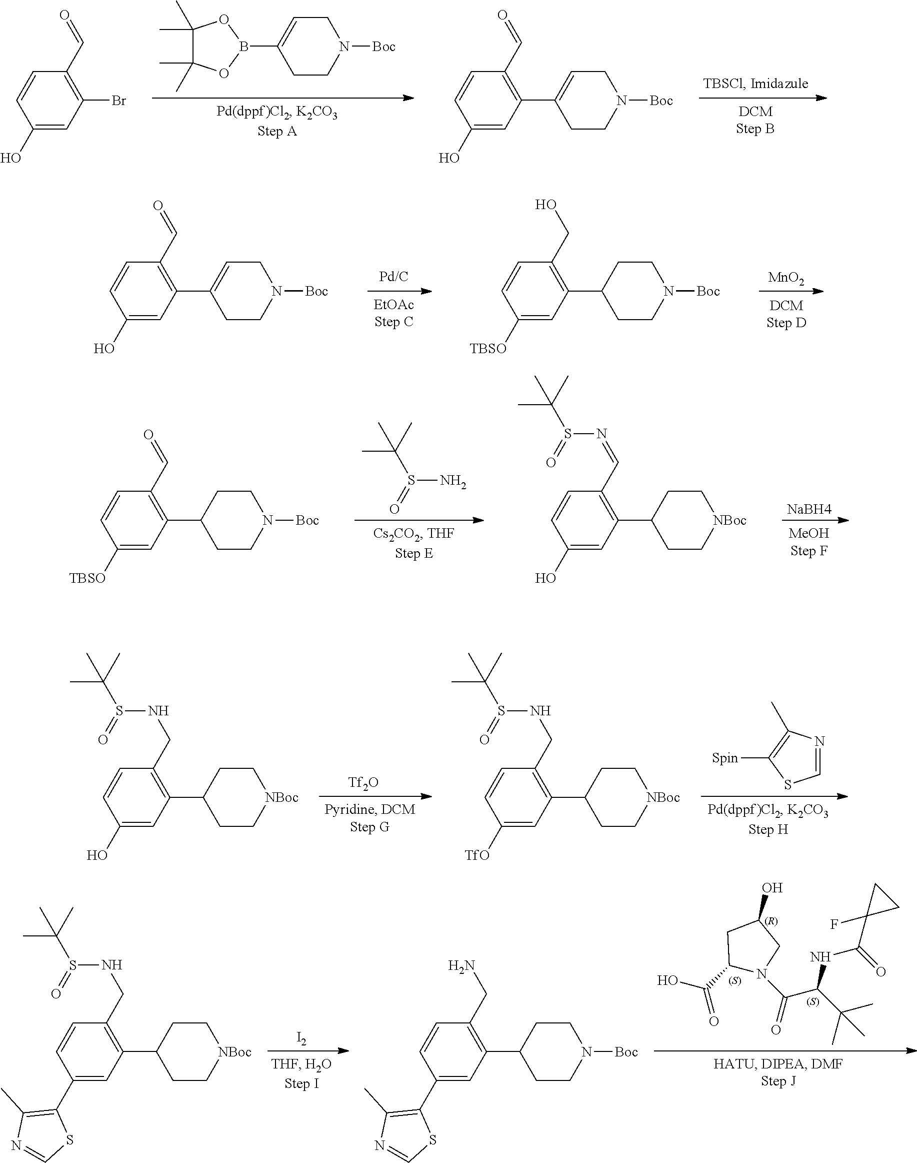

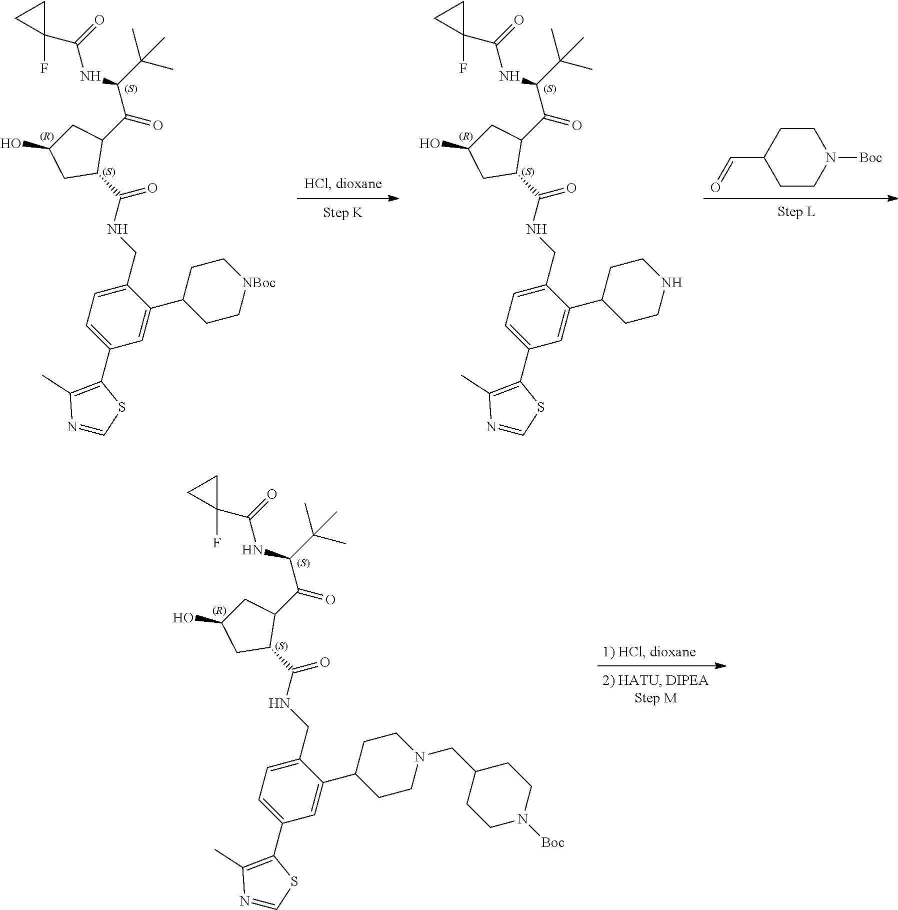

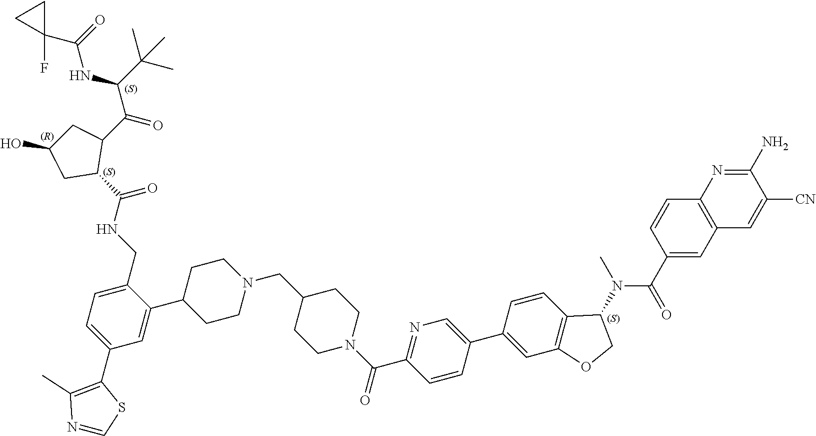

Step A: tert-butyl 4-(2-formyl-5-hydroxyphenyl)-3,6-dihydropyridine-1 (2H)-carboxylate

A mixture of 2-bromo-4-hydroxybenzaldehyde (5.0 g, 24.9 mmol, 1.0 eq.), Pd (dppf) Cl2 (1.8 g, 2.49 mmol, 0.1 eq.), tert-butyl 4-(4,4,5,5-tetramethyl-1,3,2-dioxaborolan-2-yl)-3,6-dihydropyridine-1 (2H)-carboxylate (9.2 g, 29.8 mmol, 1.2 eq.) and K₂CO₃ (10.3 g, 74.6 mmol, 3 eq.) in 1,4-Dioxane (15 mL) and H₂O (1.5 mL) was stirred at 100° C. for 2 hours under nitrogen. After cooling to room temperature, the resulting solution was diluted with 80 mL of water and extracted with EtOAc (50 mL×3). The combined organic layers were washed with brine (50 mL), dried over Na₂SO₄ and concentrated under reduced pressure. The crude product was purified by column chromatography (silica gel, 0% EtOAc in PE~30% EtOAc in PE) to afford tert-butyl 4-(2-formyl-5-hydroxyphenyl)-3,6-dihydropyridine-1 (2H)-carboxylate (6.5 g, 86% yield) as a yellow solid. LC-MS (ESI): mass calcd. For C₁₇H₂₁NO₄, 303.15; m/z found, 304.2 [M+H]⁺.

Step B: tert-butyl 4-(5-((tert-butyldimethylsilyl)oxy)-2-formylphenyl)-3,6-dihydropyridine-1 (2H)-carboxylate

A mixture of tert-butyl 4-(2-formyl-5-hydroxyphenyl)-3,6-dihydropyridine-1 (2H)-carboxylate (1.0 g, 3.30 mmol, 1.0 eq.), Imidazole (269 mg, 3.96 mmol, 1.2 eq.) and tert-Butyldimethylchlorosilane (596 mg, 3.96 mmol, 1.2 eq.) in DCM (15 mL) was stirred at room temperature for 2 hours under nitrogen. The resulting solution was diluted with 50 mL of water and extracted with EtOAc (50 mL×3). The combined organic layers were washed with brine (50 mL), dried over Na₂SO₄ and concentrated under reduced pressure. The crude product was purified by column chromatography (silica gel, 0% EtOAc in PE~10% EtOAc in PE) to afford tert-butyl 4-(5-((tert-butyldimethylsilyl)oxy)-2-formylphenyl)-3,6-dihydropyridine-1 (2H)-carboxylate (800 mg, 58% yield) as a yellow solid. LC-MS (ESI): mass calcd. For C₂₃H35NO₄Si, 417.23; m/z found, 418.1 [M+H]⁺.

Step C: tert-butyl 4-(5-((tert-butyldimethylsilyl)oxy)-2-(hydroxymethyl)phenyl)piperidine-1-carboxylate

A mixture of tert-butyl 4-(5-((tert-butyldimethylsilyl)oxy)-2-formylphenyl)-3,6-dihydropyridine-1 (2H)-carboxylate (2.0 g, 4.79 mmol, 1 eq.) and Pd/C (600 mg, 4.79 mmol, 1.0 eq.) in EtOAc (30 mL) was stirred at room temperature for 8 hours under H₂. The mixture was filtered and removed under removed pressured to afford tert-butyl 4-(5-((tert-butyldimethylsilyl)oxy)-2-(hydroxymethyl)phenyl)piperidine-1-carboxylate (2 g, 99% yield) as a yellow solid. LC-MS (ESI): mass calcd. For C₂₃H39NO₄Si, 421.26; m/z found, 422.0 [M+H]⁺.

Step D: tert-butyl 4-(5-((tert-butyldimethylsilyl)oxy)-2-formylphenyl)piperidine-1-carboxylate

A mixture of tert-butyl 4-(5-((tert-butyldimethylsilyl)oxy)-2-(hydroxymethyl)phenyl)piperidine-1-carboxylate (2.2 g, 5.22 mmol, 1.0 eq.) and manganese (IV) oxide (454 mg, 5.22 mmol, 1.0 eq.) in DCM (10 mL) was stirred at room temperature for 16 hours under nitrogen. The resulting solution was filtered and concentrated under reduced pressure to afford tert-butyl 4-(5-((tert-butyldimethylsilyl)oxy)-2-formylphenyl)piperidine-1-carboxylate (1.5 g, 68% yield) as a yellow solid. LC-MS (ESI): mass calcd. For C₂₃H₃₇NO₄Si, 419.25; m/z found, 420.0 [M+H]⁺.

Step E: tert-butyl (Z)-4-(2-(((tert-butylsulfinyl)imino)methyl)-5-hydroxyphenyl)piperidine-1-carboxylate

A mixture of tert-butyl 4-(5-((tert-butyldimethylsilyl)oxy)-2-formylphenyl)piperidine-1-carboxylate (2.0 g, 4.77 mmol, 1.0 eq.), 2-methylpropane-2-sulfinamide (866 mg, 7.15 mmol, 1.5 eq.) and Cs₂CO₃ (3.1 g, 9.53 mmol, 2.0 eq.) in THF (30 mL) was stirred at 80° C. for 32 hours under nitrogen. After cooling to room temperature, the resulting solution was diluted with water (50 mL) and extracted with EtOAc (50 mL×3). The combined organic layers were washed with brine (50 mL), dried over Na₂SO₄ and concentrated under reduced pressure. The crude product was purified by column chromatography (silica gel, 0% EtOAc in PE~80% EtOAc in PE) to afford tert-butyl (Z)-4-(2-(((tert-butylsulfinyl)imino)methyl)-5-hydroxyphenyl)piperidine-1-carboxylate (1.6 g, 82% yield) as a yellow solid. LC-MS (ESI): mass calcd. For C₂₁H32N₂O₄S, 408.21; m/z found, 409.4 [M+H]⁺.

Step F: tert-butyl 4-(5-((tert-butyldimethylsilyl)oxy)-2-(((tert-butylsulfinyl)amino)methyl)phenyl)piperidine-1-carboxylate

A mixture of tert-butyl (Z)-4-(2-(((tert-butylsulfinyl)imino)methyl)-5-hydroxyphenyl)piperidine-1-carboxylate (1.6 g, 3.92 mmol, 1.0 eq.) and NaBH₄ (1.2 g, 31.3 mmol, 8.0 eq.) in MeOH (10 mL) was stirred at room temperature for 36 hours under nitrogen. The resulting solution was diluted with water (50 mL) and extracted with EtOAc (60 mL×3). The combined organic layers were washed with brine (50 mL), dried over Na₂SO₄ and concentrated under reduced pressure. The crude product was purified by column chromatography (silica gel, 0% EtOAc in PE~100% EtOAc in PE) to afford tert-butyl 4-(2-(tert-butylsulfinyl)amino)methyl)-5-hydroxyphenyl)piperidine-1-carboxylate (500 mg, 31% yield) as a yellow solid. LC-MS (ESI): mass calcd. For C₂₁H₃₄N₂O₄S, 410.22; m/z found, 411.2 [M+H]⁺.

Step G-M: 2-amino-3-cyano-N-(S)-6-16-14-(4-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-1-yl)methyl)piperidine-1-carbonyl)pyridin-3-yl)-2,3-dihydrobenzofuran-3-yl)-N-methylquinoline-6-carboxamide

Example A241 was synthesized following the same procedures as described in Example A126 (4.7 mg, 1% yield over 7 steps) as a white solid. LC-MS (ESI): mass calcd. for C₆₃H₇₀FN₁₁O₇S, 1143.52; m/z found, 1144.7 [M+H]⁺. ¹H NMR (400 MHz, DMSO-d₆) δ 9.02 (s, 1H), 8.95-8.74 (m, 3H), 8.53 (s, 1H), 8.23 (d, J=8.8 Hz, 1H), 7.97 (s, 1H), 7.81 (d, J=8.4 Hz, 1H), 7.64 (d, J=8.0 Hz, 1H), 7.61-7.53 (m, 2H), 7.48 (d, J=8.1 Hz, 1H), 7.39 (d, J=7.1 Hz, 2H), 7.34 (s, 2H), 7.28 (s, 1H), 7.23 (d, J=8.2 Hz, 1H), 4.72-4.66 (m, 2H), 4.60-4.58 (m, 1H), 4.50-4.45 (m, 3H), 4.37-4.33 (m, 2H), 3.87-3.78 (m, 2H), 3.70-3.58 (m, 4H), 3.24-3.10 (m, 3H), 3.10-2.98 (m, 3H), 2.90-2.84 (m, 1H), 2.68 (s, 3H), 2.48 (s, 3H), 2.47-2.45 (m, 1H), 2.27-2.14 (m, 1H), 2.11-1.97 (m, 4H), 1.94-1.84 (m, 2H), 1.79-1.68 (m, 1H), 1.41-1.19 (m, 6H), 0.98 (s, 9H).

### Example A242. 3-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,1-dimethyl-1H-pyrazolo[4,3-c]quinoline-8-carboxamide

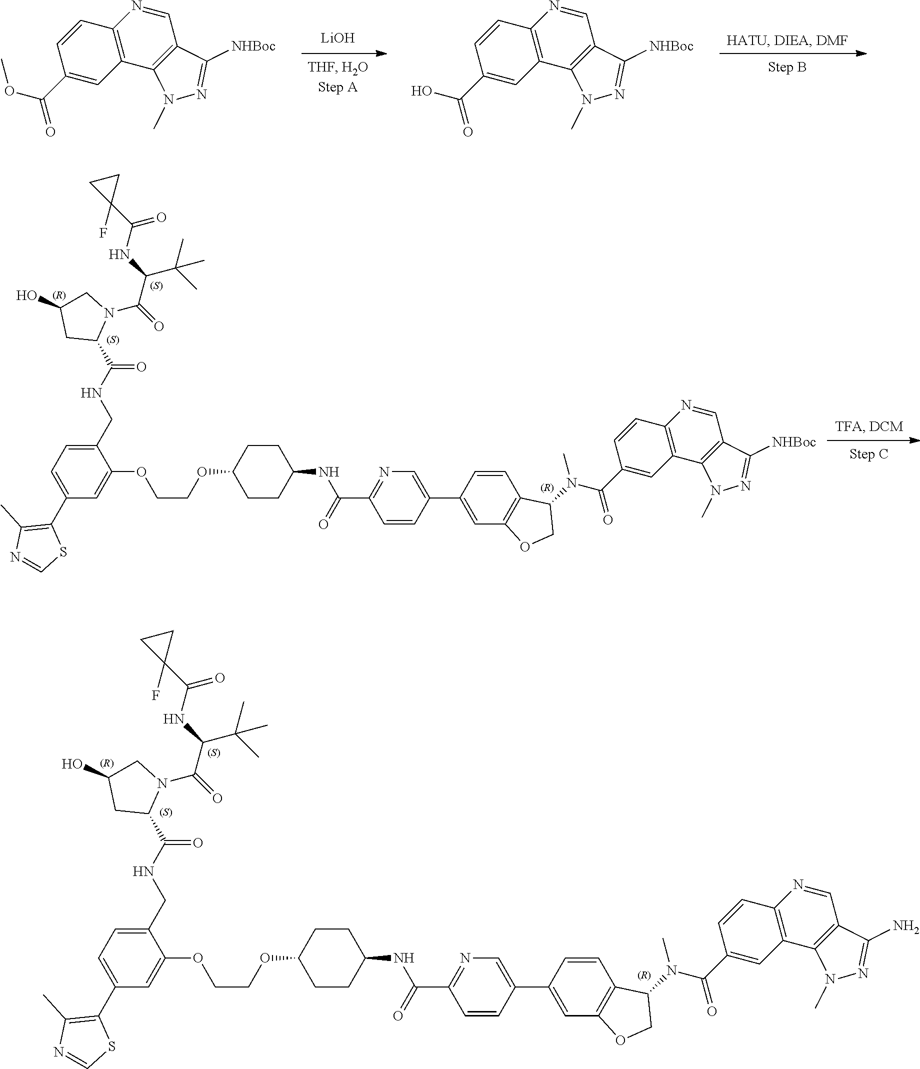

Example A242 was synthesized following the same procedures as described in Example A181 (3.2 mg, 2% yield over 3 steps) as a yellow solid. LC-MS (ESI): mass calcd. for C₆₁H₆₈FN₁₁O₉S, 1149.49; m/z found, 1150.8 [M+H]⁺. ¹HNMR (400 MHz, DMSO-d₆) δ 9.46 (s, 1H), 8.98 (s, 1H), 8.92 (s, 1H), 8.63 (d, J=19.2 Hz, 1H), 8.52-8.44 (m, 2H), 8.29-8.18 (m, 2H), 8.14-8.06 (m, 2H), 7.67-7.58 (m, 1H), 7.45-7.32 (m, 3H), 7.29 (dd, J=9.6, 2.5 Hz, 1H), 7.06-7.04 (m, 1H), 6.97 (dd, J=7.8, 1.2 Hz, 1H), 4.78 (s, 1H), 4.59 (d, J=9.5 Hz, 1H), 4.52 (t, J=8.2 Hz, 1H), 4.35 (s, 3H), 4.29 (d, J=5.8 Hz, 1H), 4.24 (d, J=5.3 Hz, 1H), 4.19-4.15 (m, 2H), 3.82-3.78 (m, 3H), 3.67-3.58 (m, 5H), 2.76 (s, 2H), 2.68-2.63 (m, 2H), 2.46 (s, 3H), 2.13-2.08 (m, 1H), 2.07-2.02 (m, 2H), 1.96-1.91 (m, 1H), 1.85 (d, J=11.1 Hz, 2H), 1.55-1.47 (m, 2H), 1.39-1.28 (m, 4H), 1.21 (dd, J=8.0, 2.9 Hz, 2H), 0.95 (s, 9H).

### Example A243. 2-amino-3-cyano-N-((S)-6-(6-(((18,4S)-4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)but-3-yn-1-yl)oxy)cyclohexyl) carbamoyl)pyridin-3-yl)-2,3-dihydrobenzofuran-3-yl)-N-methylquinoline-6-carboxamide

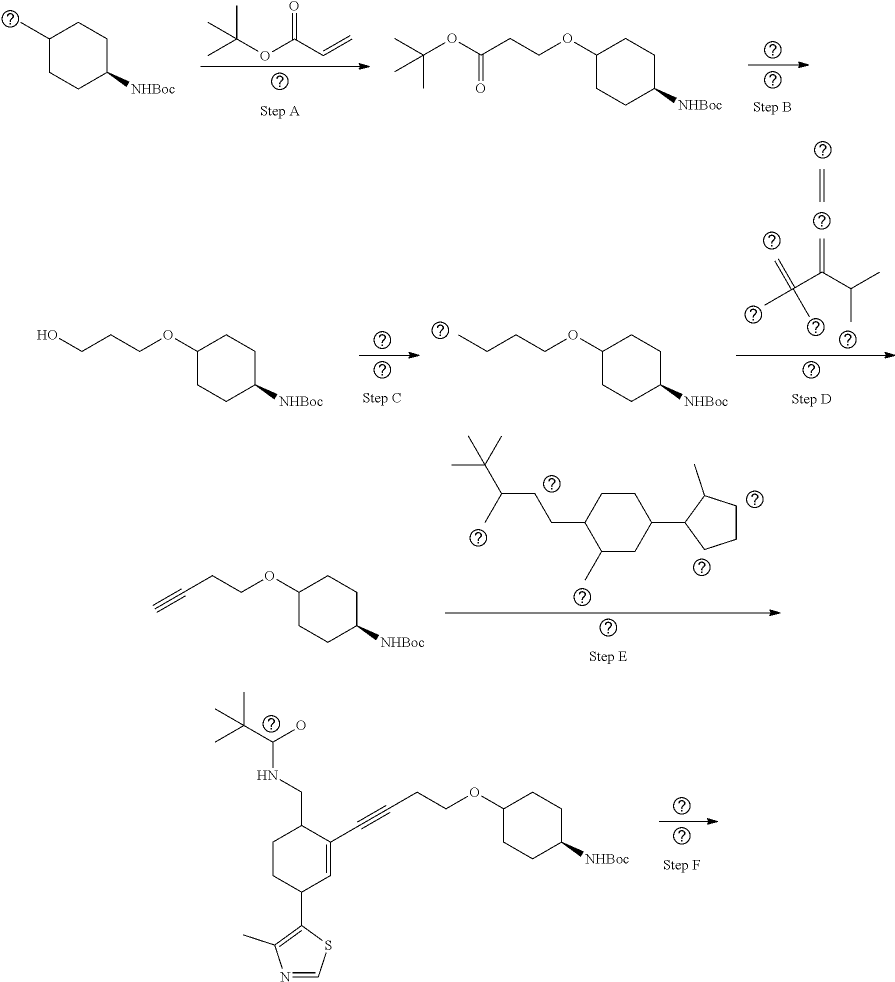

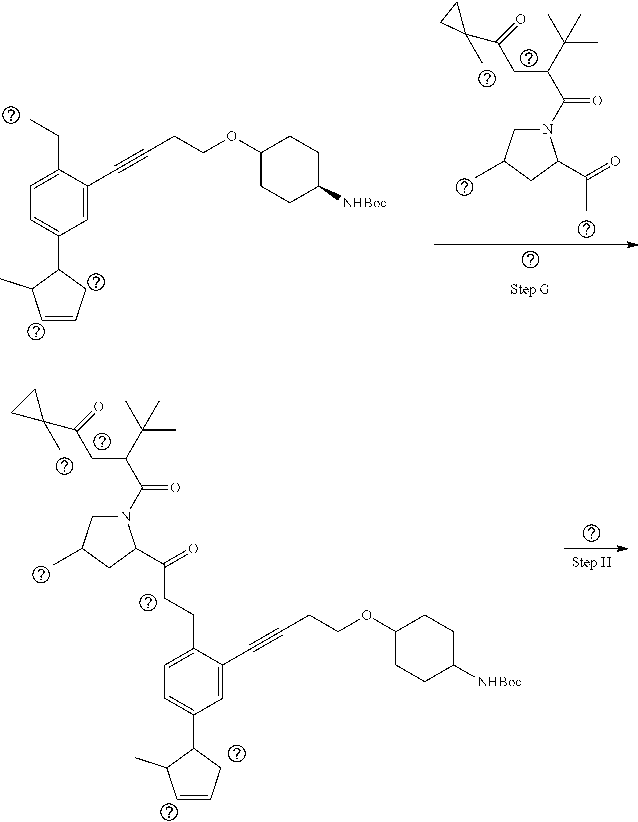

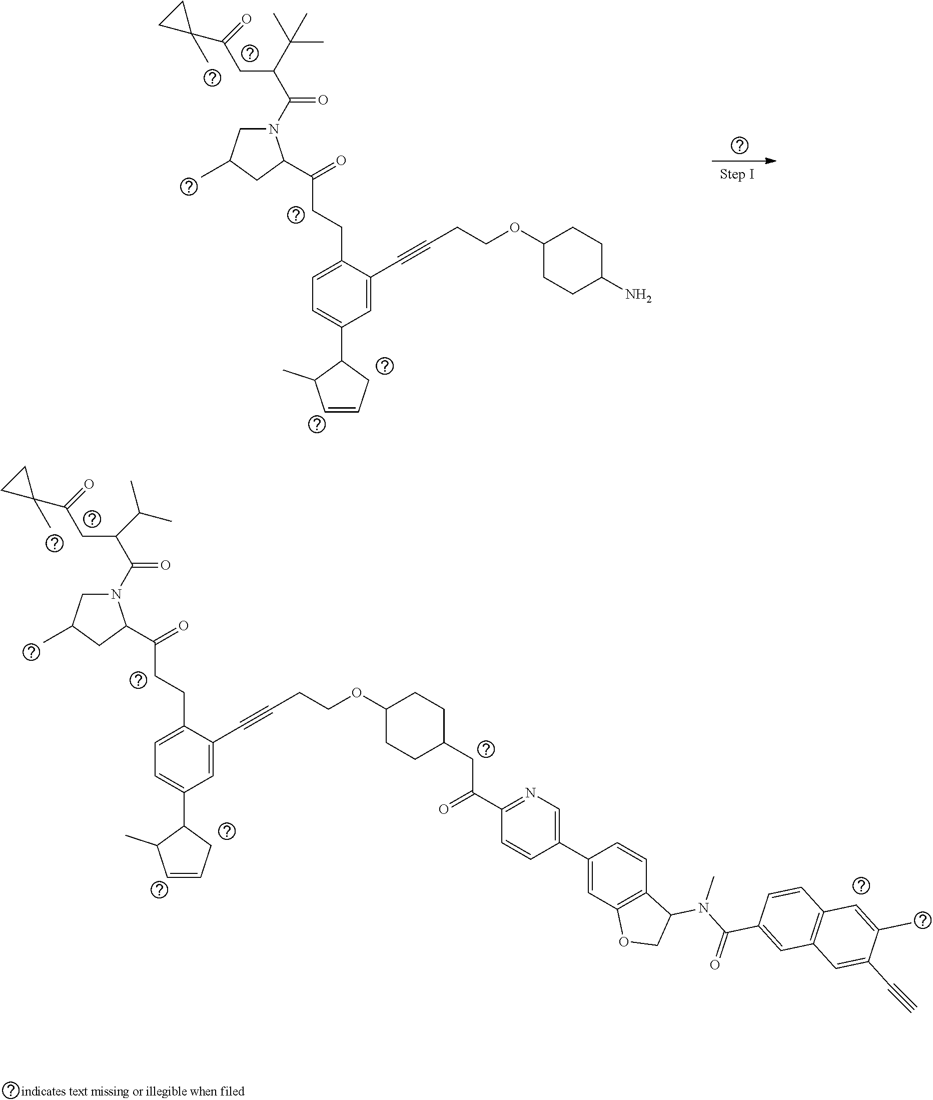 [SMILES: CC1CC(C(=O)CCc2ccc(C3CC=CC3C)cc2C#CCCOC2CCC(N)CC2)N(C(=O)C(CC(=O)C2(C)CC2)C(C)(C)C)C1 ; C#Cc1cc2cc(C(=O)N(C)C3COc4cc(-c5ccc(C(=O)CC6CCC(OCCC#Cc7cc(C8CC=CC8C)ccc7CCC(=O)C7CC(C)CN7C(=O)C(CC(=O)C7(C)CC7)C(C)C)CC6)nc5)ccc43)ccc2cc1C]

Step A: tert-butyl 3-(((1r,4r)-4-((tert-butoxycarbonyl)amino)cyclohexyl)oxy) propanoate

To a mixture of tert-butyl ((1r,4r)-4-hydroxycyclohexyl)carbamate (10.0 g, 46.4 mmol, 1.0 eq.), Hydrogen tetra (but-1-yl) ammonium sulphate (1.58 g, 4.64 mmol, 0.1 eq.), NaOH (5.57 g, 139 mmol, 3.0 eq.) in DCM (200 mL) was added 2-Propenoic acid, 1,1-dimethylethyl ester (8.93 g, 10.2 mL, 69.7 mmol, 1.5 eq.). The mixture was stirred at room temperature for 12 hours under N2 atmosphere. The resulting solution was diluted with 100 mL of water and extracted with EtOAc (100 mL×3). The combined organic layers were washed with brine, dried over anhydrous Na₂SO₄ and concentrated under reduced pressure. The residue was purified by flash column chromatography on silica gel (100-200 mesh silica gel, 0-16% EtOAc in PE) to afford tert-butyl 3-(((1r,4r)-4-((tert-butoxycarbonyl)amino)cyclohexyl)oxy) propanoate (13 g, 81% yield) as a yellow oil. LC-MS (ESI): mass calcd. For C₁₈H₃₃NO₅, 343.24; m/z found, 344.0 [M+H]⁺.

Step B: tert-butyl ((1r,4r)-4-(3-hydroxypropoxy)cyclohexyl)carbamate

To a mixture of isopropyl 3-(((1r,4r)-4-((tert-butoxycarbonyl)amino)cyclohexyl)oxy) propanoate (13.0 g, 39.5 mmol, 1.0 eq.) in THF (150 mL) was added Diisobutylaluminum hydride (11.2 g, 13.7 mL, 78.9 mmol, 2.0 eq.) at −60° C. The mixture was stirred at −60° C. for 2 hours under N₂ atmosphere. The resulting solution was diluted with 100 mL of Potassium sodium tartrate and extracted with EtOAc (100 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford tert-butyl ((1r,4r)-4-(3-oxopropoxy)cyclohexyl)carbamate (2.00 g, 18% yield) as a white oil. LC-MS (ESI): mass calcd. For C₁₄H₂₇NO₄, 273. 19; m/z found, 274.2 [M+H]⁺.

Step C: tert-butyl ((1r,4r)-4-(3-oxopropoxy)cyclohexyl)carbamate

To the solution of tert-butyl ((1r,4r)-4-(3-hydroxypropoxy)cyclohexyl)carbamate (70.0 mg, 256 μmol, 1.0 eq.) in DCM (5.00 mL) was added DMP (109 mg, 256 μmol, 1.5 eq.) and stirred for 2 hour at room temperature. The resulting solution was diluted with 10 mL of Sodium thiosulfate and extracted with DCM (30 mL×3). The combined organic layers were washed with brine, dried over anhydrous Na₂SO₄ and concentrated under reduced pressure to afford tert-butyl ((1r,4r)-4-(3-oxopropoxy)cyclohexyl)carbamate (55.0 mg, 79% yield) as a white solid. LC-MS (ESI): mass calcd. For C₁₄H₂₅NO₄, 271.18; m/z found, 272.0 [M+H]⁺.

Step D: tert-butyl ((1r,4r)-4-(but-3-yn-1-yloxy)cyclohexyl)carbamate

To a mixture of tert-butyl ((1r,4r)-4-(3-oxopropoxy)cyclohexyl)carbamate (108.0 mg, 398.0 μmol, 1.0 eq.) in MeOH (2 mL) was added K₂CO₃ (165.0 mg, 1.194 mmol, 3.0 eq.) and Bestmann-Ohira Reagent (114.7 mg, 110 μL, 597.0 μmol, 1.5 eq.) and the mixture was stirred for 12 hour at room temperature. The residue was concentrated and purified by silica gel column chromatography EA/PE=0~30% to afford the tert-butyl ((1r,4r)-4-(but-3-yn-1-yloxy)cyclohexyl)carbamate (50.0 mg, 47% yield) as a white solid. LC-MS (ESI): mass calcd. For C₁₅H₂₅NO₃, 267.18; m/z found, 268.3 [M+H]⁺.

Step E: tert-butyl ((1r,4r)-4-((4-(2-(((tert-butylsulfinyl)amino)methyl)-5-(4-methylthiazol-5-yl)phenyl)but-3-yn-1-yl)oxy)cyclohexyl)carbamate

To a mixture of 2-(((tert-butylsulfinyl)amino)methyl)-5-(4-methylthiazol-5-yl) phenyl trifluoromethanesulfonate (85.0 mg, 186 μmol, 1.0 eq.), triethylamine (188 mg, 1.86 mmol, 10.0 eq.), Bis-(triphenylphosphino)-palladous chloride (13.1 mg, 18.6 μmol, 0.1 eq.), Phosphine, tricyclohexyl-(5.22 mg, 5.82 μL, 18.6 μmol, 0.1 eq.) and CuI (3.55 mg, 18.6 μmol, 0.1 eq.) in DMF (5.00 mL) was added tert-butyl ((1r,4r)-4-(but-3-yn-1-yloxy)cyclohexyl)carbamate (50.0 mg, 187 μmol, 1.0 eq.) in DMF at 100° C. and the mixture was stirred for 2 hour at 100° C. The mixture was poured into water (30 mL) and extracted with EtOAc (30 mL×3). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford tert-butyl ((1r,4r)-4-((4-(2-(((tert-butylsulfinyl)amino)methyl)-5-(4-methylthiazol-5-yl)phenyl)but-3-yn-1-yl)oxy)cyclohexyl) carbamate (105 mg, 98% yield) as a yellow oil. LC-MS (ESI): mass calcd. For C₃₀H₄₃N₃O₄S₂, 573.27; m/z found, 574.2 [M+H]⁺.

Step F-I: 2-amino-3-cyano-N-((S)-6-(6-(((1S,4S)-4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)but-3-yn-1-yl)oxy)cyclohexyl)carbamoyl) pyridin-3-yl)-2,3-dihydrobenzofuran-3-yl)-N-methylquinoline-6-carboxamide

Example A243 was synthesized following the same procedures as described in Example A126 (4.8 mg, 7% yield over 4 steps) as a white solid. LC-MS (ESI): mass calcd. for C₆₂H₆₅FN100&S, 1128.47; m/z found, 1129.4 [M+H]⁺. ¹H NMR (400 MHz, DMSO-d₆) δ 9.00 (s, 1H), 8.92 (s, 1H), 8.77 (s, 1H), 8.68 (t, J=5.9 Hz, 1H), 8.49 (d, J=8.4 Hz, 1H), 8.27 (dd, J=8.2, 2.1 Hz, 1H), 8.08 (d, J=8.2 Hz, 1H), 7.95 (s, 1H), 7.78 (dd, J=8.7, 1.8 Hz, 1H), 7.55 (t, J=7.6 Hz, 3H), 7.43 (d, J=1.8 Hz, 1H), 7.41-7.35 (m, 2H), 7.35-7.29 (m, 2H), 7.20 (s, 2H), 5.18 (d, J=3.4 Hz, 1H), 4.91-4.72 (m, 1H), 4.71-4.67 (m, 1H), 4.60 (d, J=9.2 Hz, 1H), 4.54-4.50 (m, 1H), 4.46 (d, J=6.3 Hz, 1H), 4.39-4.32 (m, 2H), 3.81 (d, J=8.3 Hz, 1H), 3.70-3.60 (m, 4H), 2.71 (t, J=6.5 Hz, 2H), 2.67 (s, 3H), 2.45 (s, 3H), 2.12-2.00 (m, 3H), 1.97-1.91 (m, 1H), 1.86 (d, J=10.5 Hz, 2H), 1.51 (dd, J=23.6, 11.0 Hz, 2H), 1.39-1.28 (m, 4H), 1.26-1.19 (m, 3H), 0.96 (s, 9H).

### Example A244. 2-amino-3-cyano-N-((S)-6-(6-((4-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)but-2-yn-1-yl)carbamoyl) pyridin-3-yl)-2,3-dihydrobenzofuran-3-yl)-N-methylquinoline-6-carboxamide

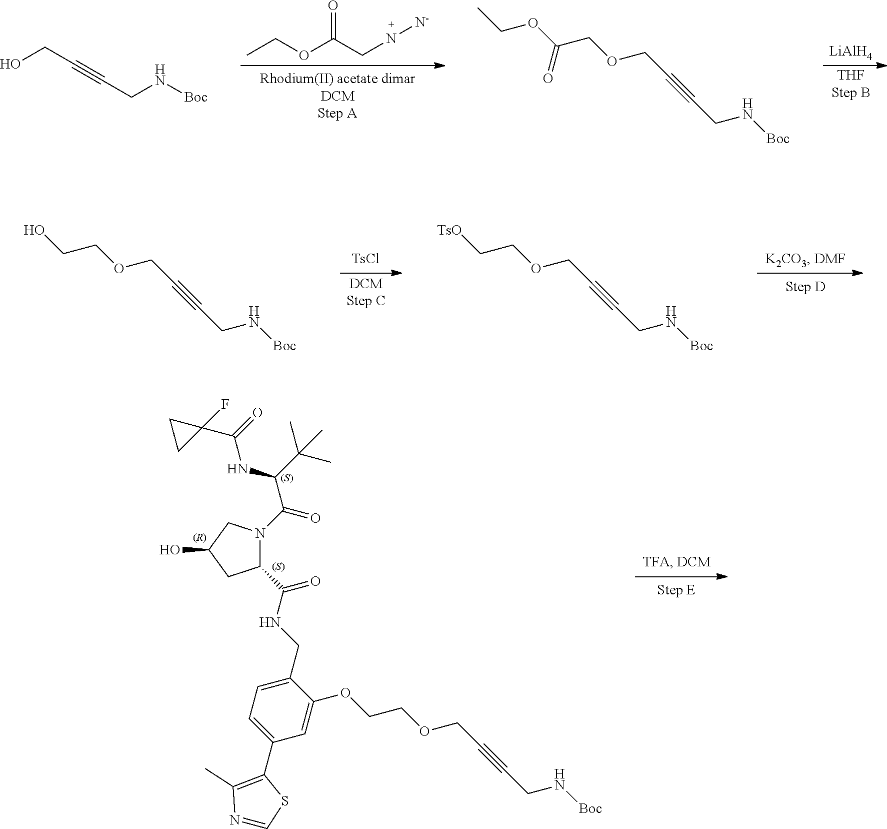

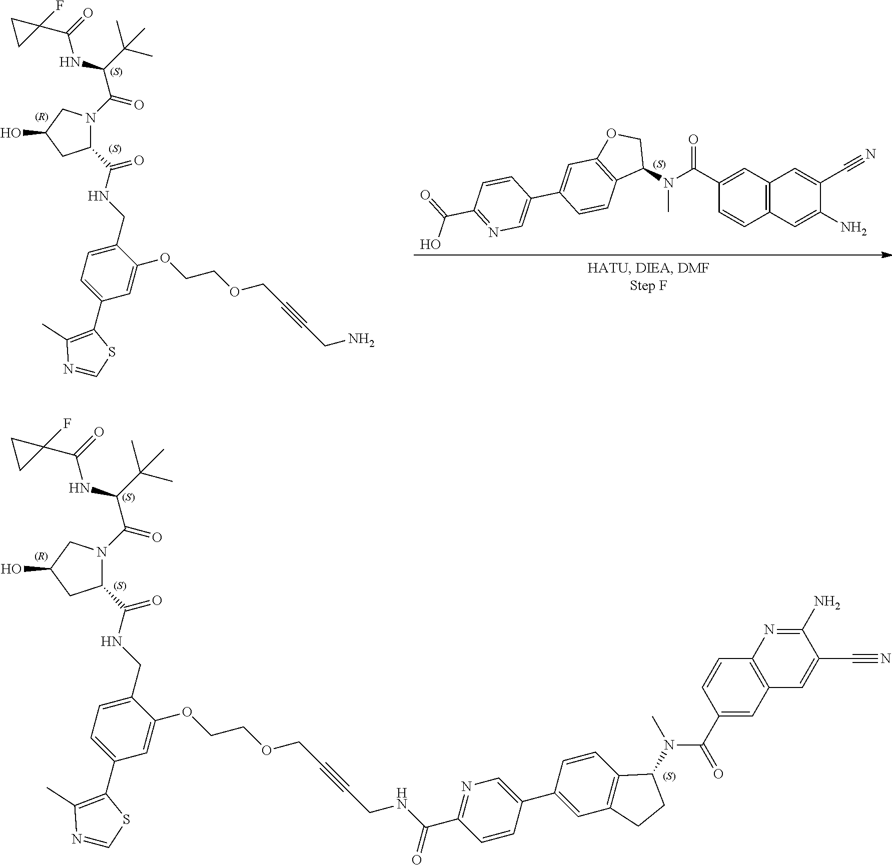

Example A244 was synthesized following the same procedures as described in Example 220 (2.0 mg, 5% yield over 6 steps) as a white solid. LC-MS (ESI): mass calcd. for C₅₈H₅₉FN₁₀O₉S, 1090.42; m/z found, 1091.7 [M+H]⁺. ¹H NMR (400 MHz, DMSO-d₆) δ 9.20-9.14 (m, 1H), 8.98 (s, 1H), 8.96-8.93 (m, 1H), 8.86 (s, 1H), 8.52-8.44 (m, 1H), 8.31-8.25 (m, 1H), 8.09 (d, J=8.2 Hz, 1H), 7.99 (s, 1H), 7.86-7.81 (m, 1H), 7.70-7.47 (m, 4H), 7.41 (d, J=7.8 Hz, 2H), 7.37 (s, 1H), 7.31-7.26 (m, 1H), 7.05-7.02 (m, 1H), 6.98-6.94 (m, 1H), 4.81-4.67 (m, 2H), 4.59 (d, J=9.2 Hz, 1H), 4.52 (t, J=8.2 Hz, 1H), 4.37-4.33 (m, 1H), 4.30-4.27 (m, 3H), 4.23-4.19 (m, 3H), 4.18-4.13 (m, 3H), 3.83-3.81 (m, 2H), 3.66-3.59 (m, 2H), 2.68 (s, 3H), 2.46 (s, 3H), 2.09-2.07 (m, 1H), 1.96-1.88 (m, 1H), 1.41-1.31 (m, 2H), 1.24-1.19 (m, 2H), 0.95 (s, 9H).

### Example A245. 2-amino-3-cyano-N-((S)-6-(6-(((E)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)but-2-en-1-yl)carbamoyl) pyridin-3-yl)-2,3-dihydrobenzofuran-3-yl)-N-methylquinoline-6-carboxamide

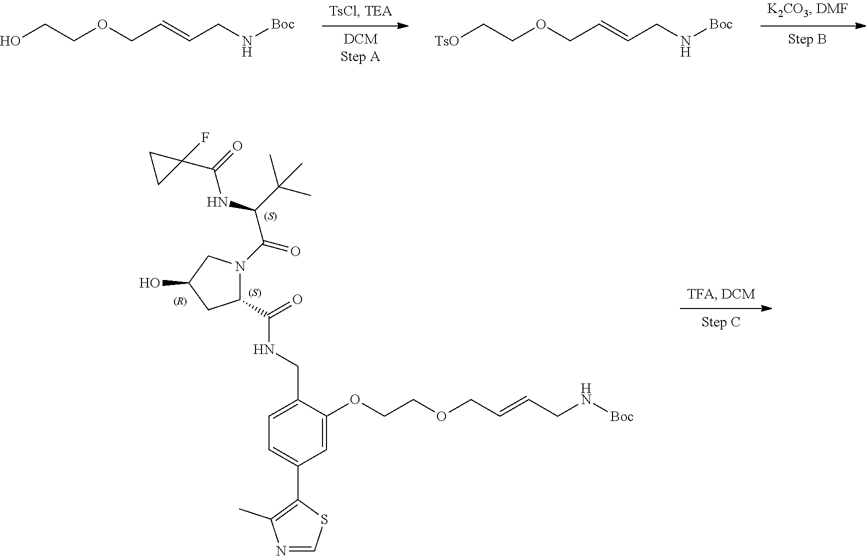

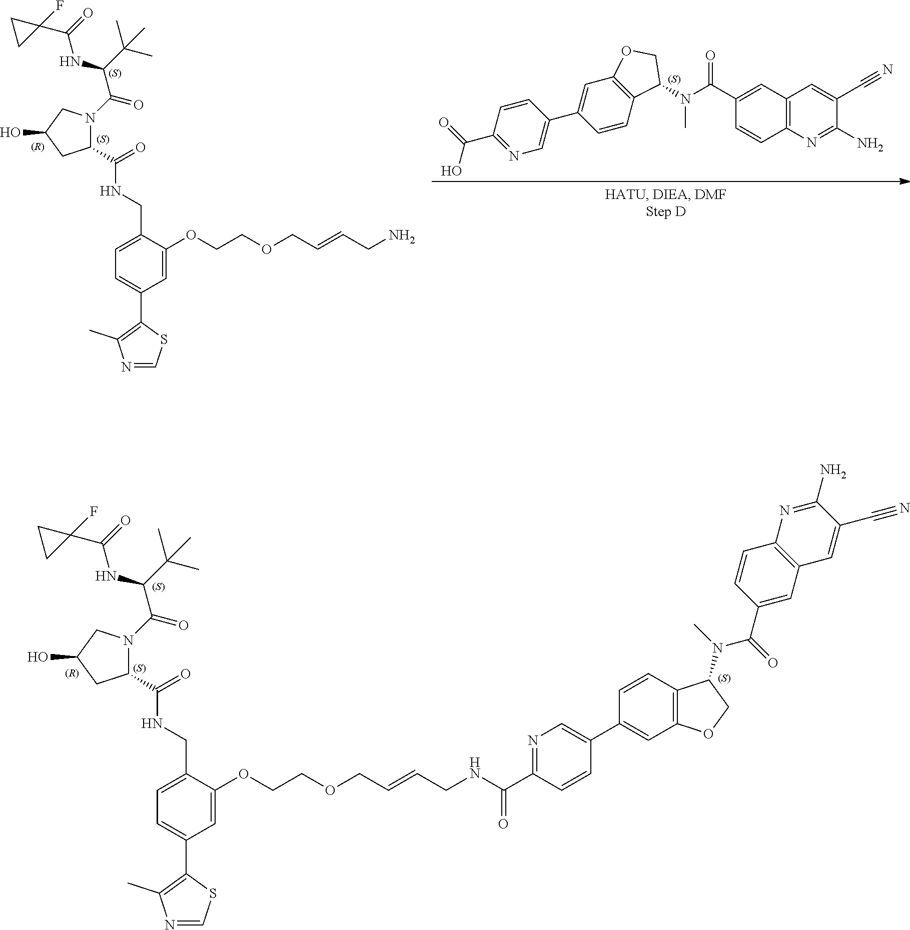

Example A245 was synthesized following the same procedures as described in Example A220 (15.0 mg, 29% yield over 4 steps) as a white solid. LC-MS (ESI): mass calcd. for C₅₈H₆₁FN₁₀O₉S, 1092.43/z found, 1093.6 [M+H]⁺. ¹H NMR (400 MHz, DMSO-d₆) δ 8.98 (s, 1H), 8.96-8.91 (m, 2H), 8.87 (s, 1H), 8.48 (t, J=5.9 Hz, 1H), 8.29-8.25 (m, 1H), 8.10-8.07 (m, 1H), 8.00 (s, 1H), 7.85 (dd, J=8.7, 1.8 Hz, 1H), 7.65-7.54 (m, 3H), 7.40 (d, J=7.8 Hz, 2H), 7.36 (s, 1H), 7.30-7.26 (m, 1H), 7.04 (s, 1H), 6.97-6.94 (m, 1H), 5.84-5.76 (m, 1H), 5.71-5.64 (m, 1H), 4.82-4.67 (m, 2H), 4.59 (d, J=9.2 Hz, 1H), 4.51 (t, J=8.2 Hz, 1H), 4.36-4.26 (m, 3H), 4.23-4.19 (m, 3H), 3.96-3.94 (m, 4H), 3.77-3.74 (m, 2H), 3.65-3.59 (m, 2H), 2.67 (s, 3H), 2.45 (s, 3H), 2.09-2.07 (m, 1H), 1.95-1.88 (m, 1H), 1.40-1.31 (m, 2H), 1.24-1.18 (m, 2H), 0.94 (s, 9H).

### Example A248. 2-amino-3-(difluoromethyl)-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

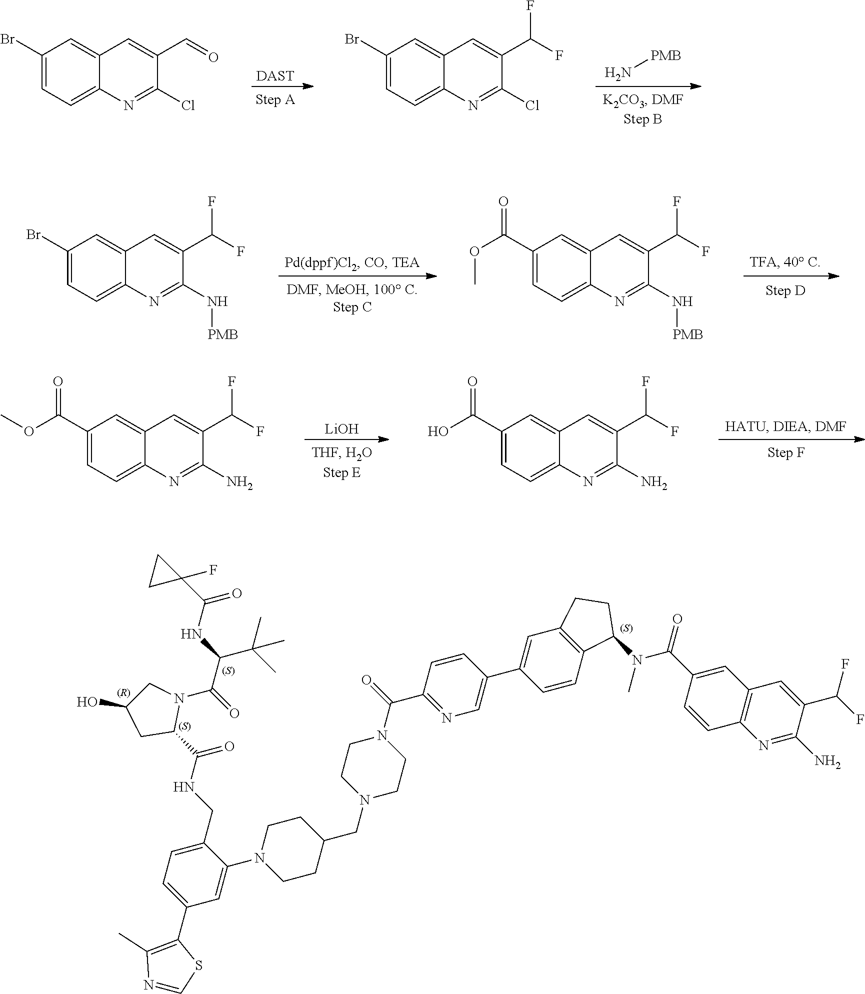

Step A: 6-bromo-2-chloro-3-(difluoromethyl)quinoline

To a solution of 6-bromo-2-chloroquinoline-3-carbaldehyde (800 mg, 2.96 mmol, 1.0 eq.) in DCM (10 mL) was added DAST (3.13 mL, 23.7 mmol, 8.0 eq.) under N₂. The mixture was stirred at room temperature for 2 hours. The reaction mixture was quenched with NH₄Cl (aq. 100 mL), then extracted with DCM (80 mL×3). The combined organic layers were washed with brine three times, dried over anhydrous Na₂SO₄ and concentrated. The residue was purified by column chromatography (silica gel, 0% EtOAc in PE~100% EtOAc in PE) to afford 6-bromo-2-chloro-3-(difluoromethyl)quinoline (800 mg, 88% yield) as a yellow solid. LC-MS (ESI): mass calcd. For C₁₀H₅BrClF₂N, 292.5; m/z found, 292/294 [M+H]⁺

Step B: tert-butyl ((Ir, 4r)-4-((3-(2-(((tert-butylsulfinyl)amino)methyl)-5-(4-methylthiazol-5-yl)phenyl) prop-2-yn-1-yl)oxy)cyclohexyl)carbamate

To a solution of 6-bromo-2-chloro-3-(difluoromethyl)quinoline (800 mg, 2.73 mmol, 1.0 eq.) in 1,4-Dioxane (10 mL) was added PMBNH₂ (1.5 g, 10.9 mmol, 4 eq.) under N₂. The mixture was stirred at 90° C. for 16 hours. The reaction mixture was quenched with water (50 mL), and then extracted with EtOAc (50 mL×3). The combined organic layers were washed with brine, dried over anhydrous Na₂SO₄ and concentrated. The residue was purified by column chromatography (silica gel, 0% EtOAc in PE~100% EtOAc in PE) to afford 6-bromo-3-(difluoromethyl)-N-(4-methoxybenzyl) quinolin-2-amine (430 mg, 38% yield) as a yellow solid.

LC-MS (ESI): mass calcd. For C₁₅H₁₅BrF₂N20, 393.2; m/z found, 393.0/395.0 [M+H]⁺.

Step C: methyl 3-(difluoromethyl)-2-((4-methoxybenzyl)amino) quinoline-6-carboxylate

To a solution of 6-bromo-3-(difluoromethyl)-N-(4-methoxybenzyl) quinolin-2-amine (240 mg, 610 μmol, 1.0 eq.) in DMF (5 mL) and MeOH (1 mL) was added Pd(dppf)Cl₂ (44.7 mg, 61.0 μmol, 0.1 eq.) and TEA (255 μL, 1.83 mmol, 3.0 eq.). The mixture was degassed with CO for 3 times and stirred at 100° C. using a CO balloon for 16 hours. The reaction mixture was quenched with water (50 mL), and then extracted with EtOAc (100 mL×2). The combined organic layers were washed with brine three times, dried over anhydrous Na₂SO₄ and concentrated. The residue was purified by column chromatography (silica gel, 0% EtOAc in PE~100% EtOAc in PE) to afford methyl 3-(difluoromethyl)-2-((4-methoxybenzyl)amino) quinoline-6-carboxylate (70 mg, 29% yield) as a yellow solid. LC-MS (ESI): mass calcd. For C20H18F₂N203, 372.4; m/z found, 373.2 [M+H]⁺.

Step D: methyl 2-amino-3-(difluoromethyl)quinoline-6-carboxylate

A solution of methyl 3-(difluoromethyl)-2-((4-methoxybenzyl)amino) quinoline-6-carboxylate (70 mg, 188 μmol, 1.0 eq.) in TFA (5 mL) was stirred at 40° C. for 16 hours to give yellow solution. The residue was concentrated to afford methyl 2-amino-3-(difluoromethyl)quinoline-6-carboxylate (14 mg, 29% yield) as a yellow oil. LC-MS (ESI): mass calcd. For C₁₂H₁₀F₂N₂O₂, 252.1; m/z found, 253.1 [M+H]⁺.

Steps E-F: 2-amino-3-(difluoromethyl)-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

Example A248 was synthesized following the same procedures as described in Example A23 (3.3 mg, 8% yield over 2 steps) as a white solid. LC-MS (ESD): mass calcd. for C₆₂H70F3N11O₇S, 1169.5; m/z found, 1170.5 [M+H]⁺, ¹H NMR (400 MHz, DMSO-d₆) δ 9.65 (s, 1H), 8.99 (s, 1H), 8.94 (s, 1H), 8.71 (s, 1H), 8.52 (s, 1H), 8.29 (d, J=8.3 Hz, 1H), 8.20 (s, 1H), 7.93 (s, 1H), 7.83-7.66 (m, 2H), 7.57 (d, J=7.7 Hz, 1H), 7.46-7.34 (m, 4H), 7.32-7.19 (m, 2H), 7.18-7.06 (m, 3H), 4.87-4.75 (m, 1H), 4.70 (s, 1H), 4.60 (d, J=9.1 Hz, 1H), 4.49 (t, J=8.2 Hz, 1H), 4.43-4.29 (m, 3H), 4.28-4.20 (m, 1H), 3.81-3.47 (m, 6H), 3.45-3.01 (m, 9H), 2.79-2.64 (m, 6H), 2.46 (s, 3H), 2.17-2.03 (m, 1H), 2.03-1.97 (m, 2H), 1.95-1.83 (m, 2H), 1.56-1.42 (m, 2H), 1.41-1.33 (m, 2H), 1.25-1.23 (m, 2H), 0.97 (s, 9H

### Example A249. N-((S)-6-(6-(((18,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N,3-dimethyl-2-(methylamino) quinoline-6-carboxamide

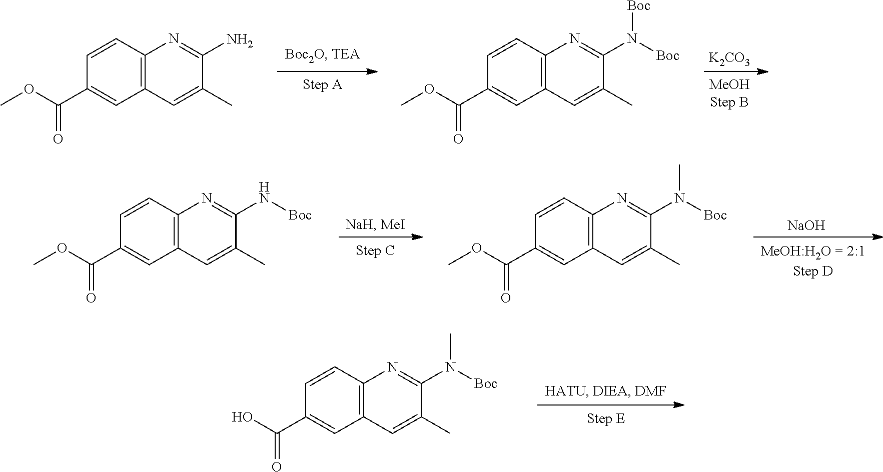

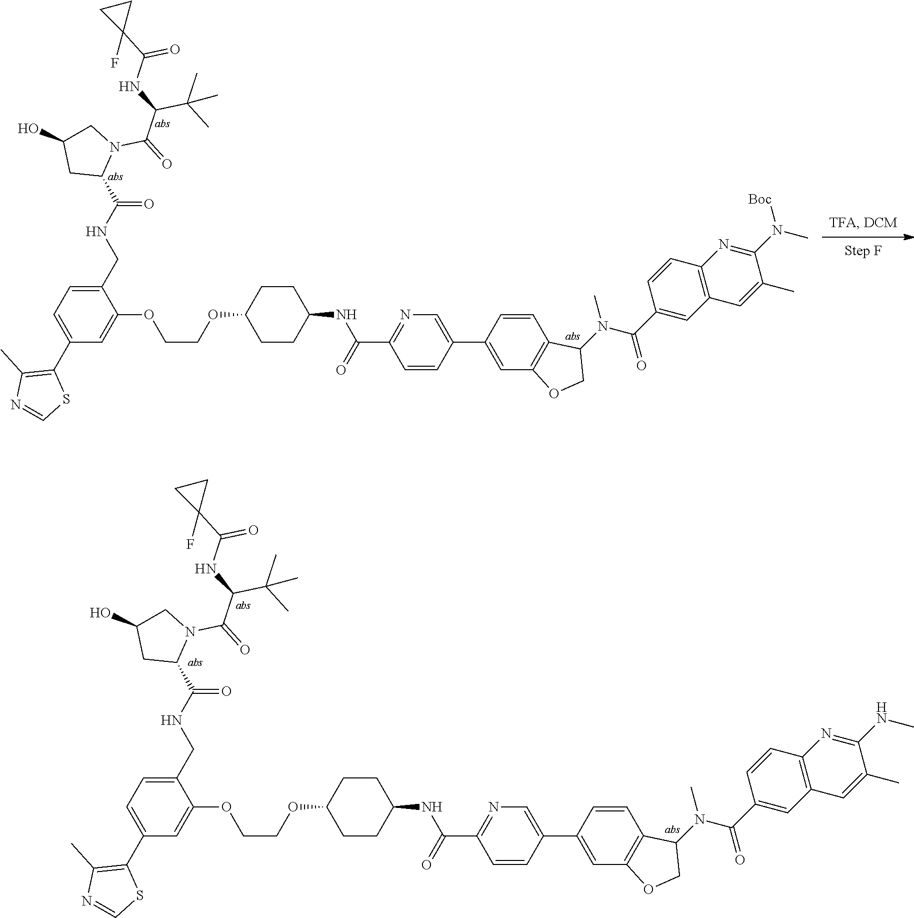

Step A: methyl 2-(bis(tert-butoxycarbonyl)amino)-3-methylquinoline-6-carboxylate

To a solution of methyl 2-amino-3-methylquinoline-6-carboxylate (2 g, 9.26 mmol, 1.0 eq.) in DCM (20 mL) was added Boc₂O (3.02 g, 13.8 mmol, 1.5 eq.) and TEA (2.56 mL, 27.78 mmol, 2.0 eq.) under N₂. The mixture was stirred at room temperature for 2 hours. The resulting solution was diluted with 100 mL of H₂O and extracted with DCM (80 mL×3). The combined organic layers were washed with brine, dried over Na₂SO₄ and concentrated under reduced pressure. The residue was purified by flash chromatography (100-200 mesh silica gel, 0%-50% EtOAc in PE) to afford methyl 2-(bis(tert-butoxycarbonyl)amino)-3-methylquinoline-6-carboxylate (3 g, 78% yield) as a yellow solid. LC-MS (ESI): mass calcd, for C₂₂H₂₈N₂O₆, 416.2; m/z found, 316.2 [M+H−56]⁺.

Step B: methyl 2-((tert-butoxycarbonyl)amino)-3-methylquinoline-6-carboxylate

To a solution of methyl 2-(bis(tert-butoxycarbonyl)amino)-3-methylquinoline-6-carboxylate (100 mg, 240 μmol, 1.0 eq.) in MeOH (10 mL) was added K₂CO₃ (99.6 mg, 720 μmol, 3.0 eq.). The mixture was stirred at 50° C. for 16 hours. The resulting solution was diluted with 30 mL of H₂O and extracted with EtOAc (30 mL×3). The combined organic layers were washed with brine, dried over Na₂SO₄ and concentrated under reduced pressure. The residue was purified by flash chromatography (100-200 mesh silica gel, 0%-50% EtOAc in PE) to afford methyl 2-((tert-butoxycarbonyl)amino)-3-methylquinoline-6-carboxylate (45 mg, 56% yield) as a yellow solid. LC-MS (ESI): mass calcd, for C₁₇H₂₀N₂O₄, 316.2; m/z found, 216.2 [M+H−56]⁺.

Step C: methyl 2-((tert-butoxycarbonyl)(methyl)amino)-3-methylquinoline-6-carboxylate

To a solution of methyl 2-((tert-butoxycarbonyl)amino)-3-methylquinoline-6-carboxylate (45.0 mg, 142 μmol, 1.0 eq.) in DMF (5 mL) was added NaH (6.83 mg, 284 μmol, 2.0 eq.) at 0° C. The mixture was stirred at 0° C. for 30 mins, then Mel (26.7 μL, 427 μmol, 3.0 eq.) was added. The mixture was stirred at room temperature for 1 hour. The mixture was quenched water (30 mL), extracted with EtOAc (30 mL×3). The combined organic layers were washed with brine, dried over Na₂SO₄ and concentrated under reduced pressure to afford methyl 2-((tert-butoxycarbonyl)(methyl)amino)-3-methylquinoline-6-carboxylate (33 mg, 67% yield) as a yellow solid. LC-MS (ESI): mass calcd, for C₁₈H₂₂N₂O₄, 330.2; m/z found, 275.2 [M+H−56]⁺.

Steps D-F: N-(S)-6-(6-(((15,4S)-4-(2-(2-(((25, 4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N,3-dimethyl-2-(methylamino) quinoline-6-carboxamide

Example A249 was synthesized following the same procedures as described in Example A51 (1.2 mg, 1% yield over 3 steps) as a white solid. LC-MS (ESI): mass calcd. for C₆₁H₇₀FN₉O₉S, 1123.5/z found, 1124.5 [M+H]⁺. ¹H NMR (400 MHz, DMSO-d₆)) δ 8.91 (d, J=9.0 Hz, 2H), 8.67-8.49 (m, 1H), 8.18 (t, J=9.2 Hz, 3H), 8.02 (s, 2H), 7.96-7.83 (m, 1H), 7.52 (dd, J=14.6, 8.2 Hz, 3H), 7.38 (s, 1H), 7.27 (s, 1H), 7.18-6.98 (m, 2H), 4.79-4.74 (m, 2H), 4.68-4.61 (m, 1H), 4.60-4.42 (m, 4H), 4.30-4.19 (m, 2H), 3.98-3.91 (m, 3H), 3.91-3.76 (m, 2H), 3.62-3.42 (m, 2H), 3.31 (s, 3H), 2.79 (s, 3H), 2.52 (s, 3H), 2.42 (s, 3H), 2.32-2.13 (m, 4H), 2.12-2.00 (m, 4H), 1.63-1.41 (m, 6H), 1.05 (s, 9H).

### Example A250. 4-amino-N-((S)-6-(6-(((18,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methylpyrazolo[1,5-a]quinoxaline-8-carboxamide

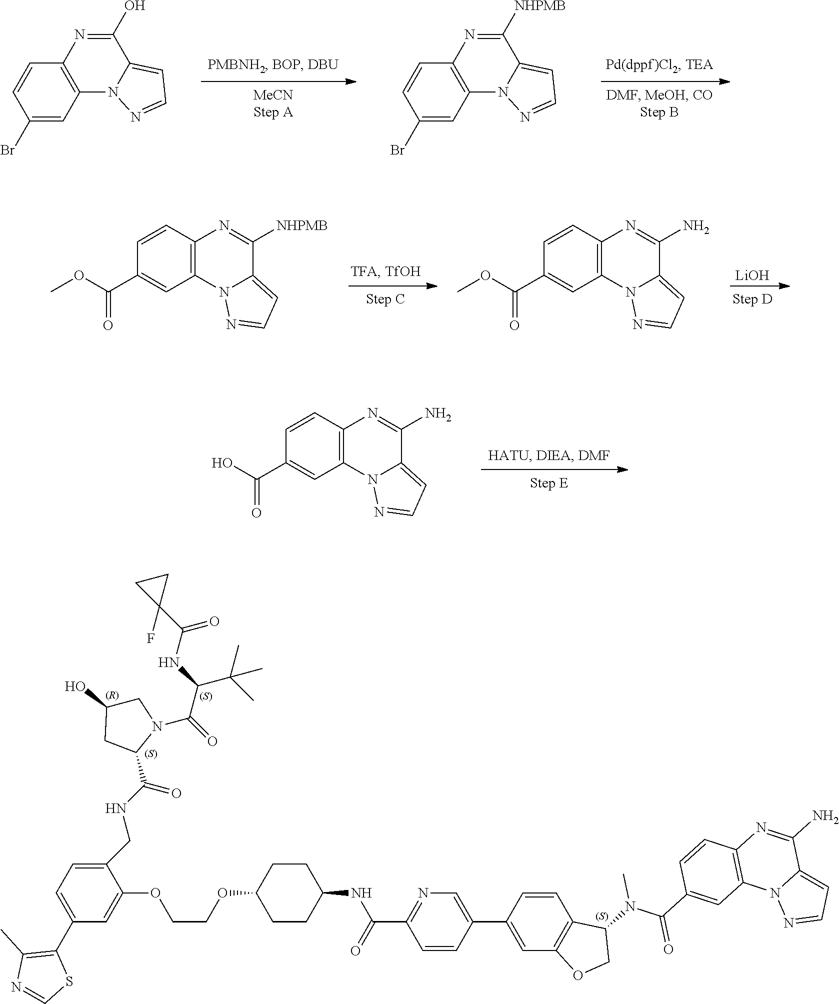

Step A: 8-bromo-N-(4-methoxybenzyl)pyrazolo[1,5-a]quinoxalin-4-amine

To a solution of 8-bromopyrazolo[1,5-a]quinoxalin-4-ol (200 mg, 757 μmol, 1.0 eq.) in MeCN (10 mL) was added Benzenemethanamine, 4-methoxy (260 mg, 1.89 mmol, 2.5 eq.), Benzotriazol-1-yloxy) tris (diMethylaMino)phosphoniuM hexafluorophosphate (670 mg, 1.51 mmol, 2.0 eq.) and 1,8-Diazabicyclo[5.4.0]undec-7-ene (576 mg, 3.79 mmol, 5.0 eq.) at room temperature. The mixture was then stirred at 50° C. for 16 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~15% EtOAc in PE) to afford 8-bromo-N-(4-methoxybenzyl)pyrazolo[1,5-a]quinoxalin-4-amine (91 mg, 31% yield) as a yellow oil. LC-MS (ESI): mass calcd. For C₁₅H₁₅BrN₄O, 382.0/384.0; m/z found, 383.0/385.0 [M+H]⁺.

Step B: methyl 4-((4-methoxybenzyl)amino)pyrazolo[1,5-a]quinoxaline-8-carboxylate

To a solution of 8-bromo-N-(4-methoxybenzyl)pyrazolo[1,5-a]quinoxalin-4-amine (90 mg, 235 μmol, 1.0 eq.) in MeOH (4 mL) and DMF (2 mL) was added TEA (71 mg, 705 μmol, 3.0 eq.) and 1,1′-Bis(diphenylphosphino) ferrocene-palladium (II) dichloride (17 mg, 23.5 μmol, 0.1 eq.) at room temperature. The mixture was then stirred at 90° C. for 16 hours under CO atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~30% EtOAc in PE) to afford methyl 4-((4-methoxybenzyl)amino)pyrazolo[1,5-a]quinoxaline-8-carboxylate (67 mg, 78% yield) as a yellow oil. LC-MS (ESI): mass calcd. For C₂₀H₁₈N₄O₃, 362.1; m/z found, 363.1 [M+H]⁺.

Step C: methyl 4-aminopyrazolo[1,5-a]quinoxaline-8-carboxylate

To a solution of methyl 4-((4-methoxybenzyl)amino)pyrazolo[1,5-a]quinoxaline-8-carboxylate (67 mg, 185 μmol, 1.0 eq.) in TFA (4 mL) and TOH (0.1 mL) was added at room temperature. The mixture was then stirred at 60° C. for 1 hour under N₂ atmosphere. The resulting solution was diluted with 30 mL of NaHCO₃/H2O and extracted with EtOAc (30 mL×2). The combined organic layers were washed with brine, dried over anhydrous Na₂SO₄, concentrated under reduced pressure. The residue was concentrated under reduced pressure to afford methyl 4-aminopyrazolo[1,5-a]quinoxaline-8-carboxylate (44 mg, 98% yield) as a yellow solid. LC-MS (ESI): mass calcd. For C₁₂H₁₀N₄O₂, 242.1; m/z found, 243.0 [M+H]⁺.

Step D-E: 4-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylpyrazolo[1,5-a]quinoxaline-8-carboxamide

Example A250 was synthesized following the same procedures as described in Example A114 (3 mg, 7% yield over 2 steps) as a white solid. LC-MS (ESI): mass calcd. for C₆₀H₆₆FN₁₁O₉S, 1135.5/z found, 1136.2 [M+H]⁺. ¹H NMR (400 MHz, DMSO-d₆) δ 8.99 (s, 1H), 8.93 (s, 2H), 8.49 (d, J=7.6 Hz, 2H), 8.37 (d, J=1.6 Hz, 1H), 8.28 (d, J=6.6 Hz, 2H), 8.08 (d, J=8.2 Hz, 1H), 7.71 (d, 1H), 7.66 (d, J=8.3 Hz, 1H), 7.60 (d, J=7.2 Hz, 1H), 7.51 (s, 1H), 7.45-7.38 (m, 2H), 7.35 (s, 1H), 7.29 (d, J=9.5 Hz, 1H), 7.05 (d, J=1.3 Hz, 1H), 6.97 (d, J=7.9 Hz, 1H), 6.40 (s, 1H), 4.77-4.74 (m, 1H), 4.61-4.58 (m, 1H), 4.54-4.51 (m, 1H), 4.38-4.35 (m, 2H), 4.34-4.33 (m, 1H), 4.31-4.29 (m, 1H), 4.25-4.24 (m, 1H), 4.22-4.20 (m, 1H), 4.19-4.17 (m, 2H), 3.82-3.80 (m, 2H), 3.68-3.64 (m, 1H), 3.63-3.59 (m, 1H), 3.40-3.33 (m, 1H), 2.68 (s, 3H), 2.47 (s, 3H), 2.14-1.99 (m, 3H), 1.98-1.80 (m, 3H), 1.58-1.45 (m, 2H), 1.41-1.26 (m, 4H), 1.24-1.19 (m, 2H), 0.96 (s, 9H).

### Example A251. 2-amino-3-cyano-N-((S)-6-(6-(((1S,4S)-4-(5-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-pyrazol-1-yl)cyclohexyl)carbamoyl)pyridin-3-yl)-2,3-dihydrobenzofuran-3-yl)-N-methylquinoline-6-carboxamide

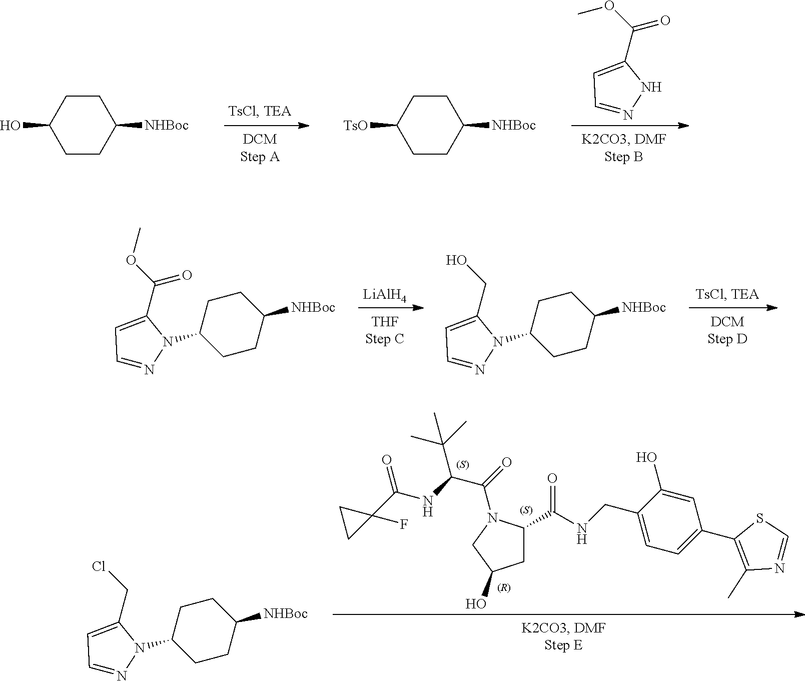

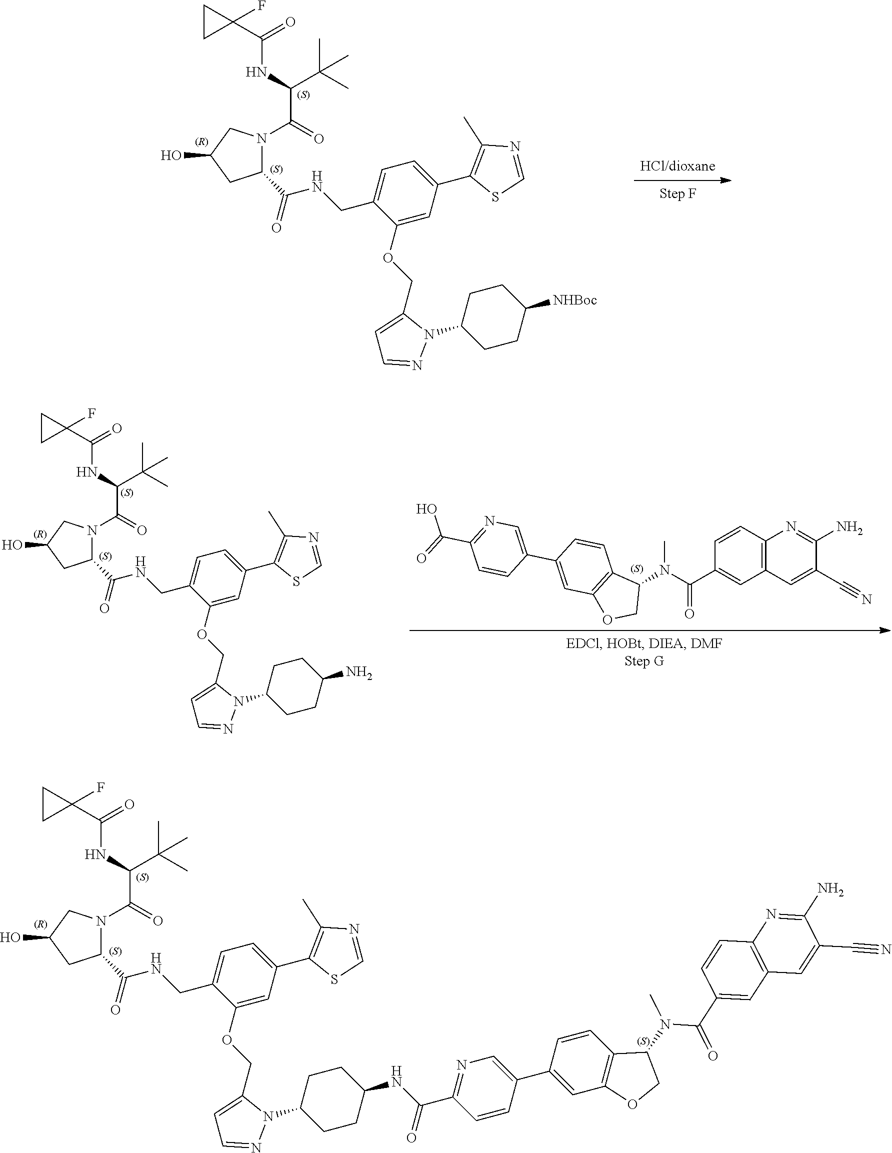

Step A: (1s,4s)-4-((tert-butoxycarbonyl)amino)cyclohexyl 4-methylbenzenesulfonate

To a stirred mixture of tert-butyl ((1s,4s)-4-hydroxycyclohexyl)carbamate (1.0 g, 4.64 mmol, 1.0 eq.) in DCM (20 mL) were added TEA (1.2 g, 1.62 mL, 11.6 mmol, 2.5 eq.), Ts-Cl (1.1 g, 5.57 mmol 1.2 eq.). DMAP (290 mg, 2.32 mmol, 0.5 eq.) was then added and the mixture was stirred at room temperature for 6 hours. The reaction mixture was diluted with DCM (50 mL), washed with brine, and organic layer was separated. The aqueous layer was extracted with DCM (80 mL×3), then the combined extracts were dried over anhydrous Na₂SO₄, filtered and evaporated. The crude product was purified by flash silica chromatography, eluting with a gradient of 0~3% MeOH in DCM to afford (1s,4s)-4-((tert-butoxycarbonyl)amino)cyclohexyl 4-methylbenzenesulfonate (1.00 g, 58% yield) as a yellow solid. LC-MS (ESI): mass calcd. For C₁₅H27NOS, 369.16; m/z found, 370.2 [M+H]⁺.

Step B: methyl 1-((1r,4r)-4-((tert-butoxycarbonyl)amino)cyclohexyl)-1H-pyrazole-5-carboxylate

To a solution of methyl 1H-pyrazole-5-carboxylate (100 mg, 793 μmol, 1.0 eq.), (1s,4s)-4-((tert-butoxycarbonyl)amino)cyclohexyl 4-methylbenzenesulfonate (290 mg, 793 μmol, 1.0 eq.) in DMF (8 mL) was added K₂CO₃ (270 mg, 1.98 mmol, 2.5 eq.). The mixture was stirred at 80° C. for 16 hours. The resulting solution was diluted with water (20 mL) and extracted with EtOAc (30 mL×3). The combined organic layers were washed with brine (50 mL), dried over Na₂SO₄ and concentrated under reduced pressure. The residue was purified by flash column chromatography on silica gel (100-200 mesh silica gel, 0-50% EtOAc in PE) to give methyl 1-((1r,4r)-4-((tert-butoxycarbonyl)amino)cyclohexyl)-1H-pyrazole-5-carboxylate (140 mg, 54% yield) as a solid.

LC-MS (ESI): mass calcd. For C₁₆H₂₅N₃O₄, 323.18; m/z found, 324.2 [M+H]⁺.

Step C: tert-butyl ((1r,4r)-4-(5-(hydroxymethyl)-1H-pyrazol-1-yl)cyclohexyl)carbamate

Under an argon atmosphere, methyl 1-((1r,4r)-4-((tert-butoxycarbonyl)amino)cyclohexyl)-1H-pyrazole-5-carboxylate (140 mg, 433 μmol, 1.0 eq.) was added LAH (33.0 mg, 866 μmol, 2.0 eq.) in dry THF (8 mL) at 0° C. The mixture is stirred for 3 hours. The reaction mixture was quenched with NH₄Cl (aq.,20 mL), and then extracted with DCM (20 mL×2). The combined organic layers were washed with brine, dried over anhydrous Na₂SO₄ and concentrated. The residue was purified by column chromatography (silica gel, 0%~10% MeOH in DCM) to afford tert-butyl ((1r,4r)-4-(5-(hydroxymethyl)-1H-pyrazol-1-yl)cyclohexyl)carbamate (70.0 mg, 55% yield) as an oil. LC-MS (ESI): mass calcd. For C₁₅H₂₅N₃O₃, 295.19; m/z found, 296.2 [M+H]⁺.

Step D: tert-butyl ((1r,4r)-4-(5-(chloromethyl)-1H-pyrazol-1-yl)cyclohexyl)carbamate

To a stirred mixture of tert-butyl ((1r,4r)-4-(5-(hydroxymethyl)-1H-pyrazol-1-yl)cyclohexyl)carbamate (70.0 mg, 237 μmol, 1.0 eq.) in DCM (5 mL) were added TEA (72.0 mg, 99.1 μL, 711 μmol, 3.0 eq.), Ts-Cl (54.0 mg, 284 μmol, 1.2 eq.). DMAP (6.0 mg, 47.4 μmol, 0.2 eq.) was then added and the mixture was stirred at room temperature for 3 hours. The reaction mixture was quenched with water (50 mL), and then extracted with EtOAc (50 mL×3). The combined organic layers were washed with brine, dried over anhydrous Na₂SO₄ and concentrated. The crude product was purified by flash silica chromatography, eluting with a gradient of 0~3% MeOH in DCM to afford tert-butyl ((1r,4r)-4-(5-(chloromethyl)-1H-pyrazol-1-yl)cyclohexyl)carbamate (50.0 mg, 67% yield) as a white solid. LC-MS (ESI): mass calcd. For C₁₅H₂₄ClN₃O₂, 313.16; m/z found, 314.2 [M+H]⁺.

Step E_G: 2-amino-3-cyano-N-((S)-6-(6-(((1S,4S)-4-(5-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-pyrazol-1-yl)cyclohexyl) carbamoyl)pyridin-3-yl)-2,3-dihydrobenzofuran-3-yl)-N-methylquinoline-6-carboxamide

Example A251 was synthesized following the same procedures as described in Example A23 (7.3 mg, 14% yield over 3 steps) as a white solid. LC-MS (ESI): mass calcd. for C₆₂H₆₈FN₁₂O₈S, 1156.48/z found, 1157.8 [M+H]⁺, ¹H NMR (400 MHz, DMSO-d₆) δ 9.01 (s, 1H), 8.93-8.91 (m, 1H), 8.83 (s, 1H), 8.58-8.52 (m, 2H), 8.30-8.26 (m, 1H), 8.09 (d, J=8.1 Hz, 1H), 7.97 (s, 1H), 7.84-7.80 (m, 1H), 7.62-7.54 (m, 2H), 7.48-7.42 (m, 3H), 7.39 (d, J=8.0 Hz, 1H), 7.34 (s, 1H), 7.28-7.23 (m, 2H), 7.02-6.98 (m, 1H), 6.41-6.39 (m, 1H), 5.35 (s, 2H), 4.83-4.66 (m, 2H), 4.56 (d, J=9.3 Hz, 1H), 4.52-4.48 (m, 1H), 4.34-4.26 (m, 3H), 4.22-4.16 (m, 1H), 3.93-3.88 (m, 2H), 3.62-3.56 (m, 2H), 2.67 (s, 3H), 2.47 (s, 3H), 2.07-1.89 (m, 8H), 1.74-1.64 (m, 2H), 1.38-1.30 (m, 2H), 1.23-1.18 (m, 2H), 0.91 (s, 9H).

### Example A252. 2-amino-3-cyano-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-isopropylquinoline-6-carboxamide

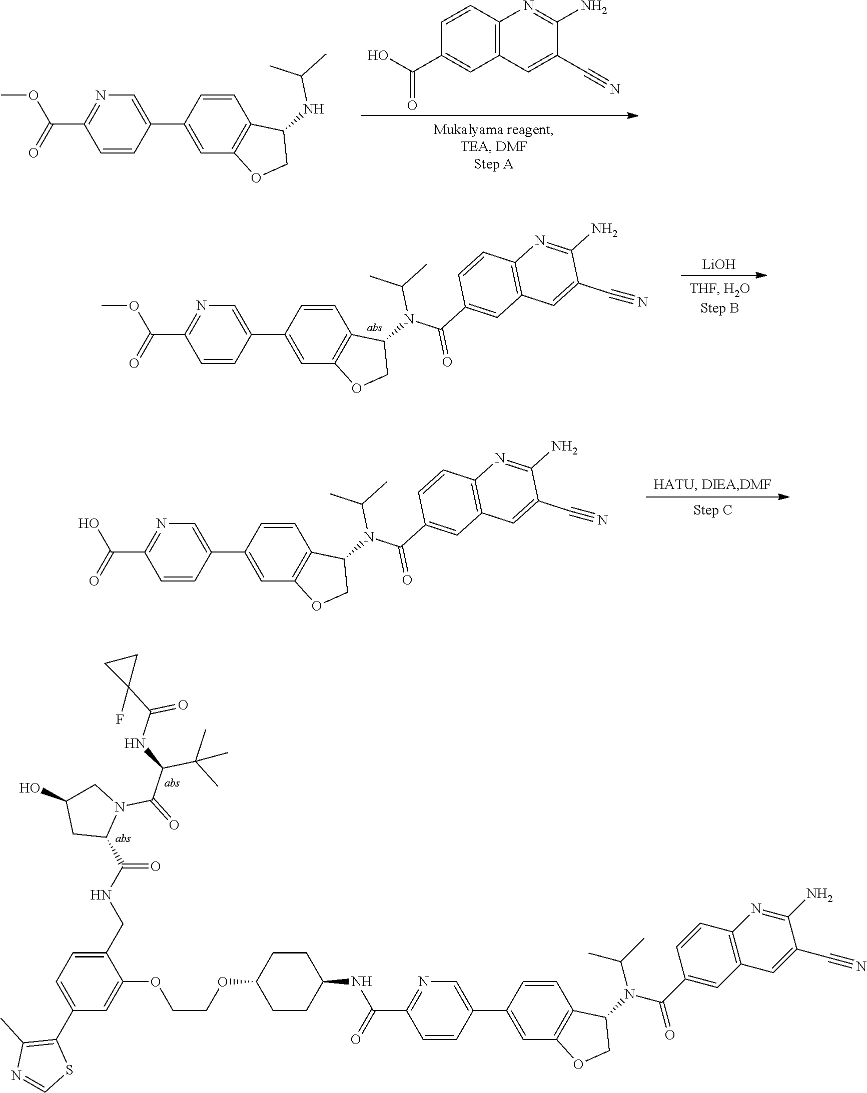

Step A: methyl(S)-5-(3-(2-amino-3-cyano-N-isopropylquinoline-6-carboxamido)-2,3-dihydrobenzofuran-6-yl)picolinate

To a stirred mixture of methyl(S)-5-(3-(isopropylamino)-2,3-dihydrobenzofuran-6-yl)picolinate (50 mg, 0.16 mmol, 1.0 eq.) in DMF (5 mL) were added TEA (48 mg, 0.07 mL, 0.48 mmol, 3.0 eq.) and 2-Chloro-1-methylpyridinium iodide (61 mg, 0.24 mmol, 1.5 eq.). The mixture was stirred at room temperature for 1 hours. The reaction mixture was diluted with Water (50 mL), extracted with EtOAc (50 mL×3), then the combined extracts were dried over anhydrous Na₂SO₄, filtered and evaporated. The crude product was purified by flash silica chromatography, eluting with a gradient of 0~3% MeOH in DCM) to methyl(S)-5-(3-(isopropylamino)-2,3-dihydrobenzofuran-6-yl)picolinate (30 mg, 37% yield) as a yellow solid. LC-MS (ESI): mass calcd. For C₂₉H₂₅N₅O₄, 507.19; m/z found, 507.3 [M+H]⁺.

Step B-C: 2-amino-3-cyano-N-(S)-6-(6-(15,4S)-4-(2-(2-(((28,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-isopropylquinoline-6-carboxamide

Example A252 was synthesized following the same procedures as described in Example A45 (1.0 mg, 1% yield over 6 steps) as a white solid. LC-MS (ESI): mass calcd. for C₆₂H6>FN₁₀O₉S, 1148.50; m/z found, 1149.8 [M+H]⁺. ¹HNMR (400 MHz, DMSO-d₆) δ 8.99 (s, 1H), 8.89 (s, 1H), 8.81 (s, 1H), 8.52-8.45 (m, 2H), 8.23 (d, J=8.1 Hz, 1H), 8.07 (d, J=8.2 Hz, 1H), 7.81 (s, 1H), 7.66 (s, 1H), 7.56 (d, J=7.1 Hz, 1H), 7.47-7.34 (m, 3H), 7.31-7.25 (m, 2H), 7.23-7.17 (m, 1H), 7.05 (d, J=1.1 Hz, 1H), 6.99-6.96 (m, 1H), 5.42 (s, 1H), 4.79-4.73 (m, 1H), 4.71-4.67 (m, 1H), 4.60 (d, J=9.3 Hz, 1H), 4.52 (t, J=8.1 Hz, 1H), 4.36 (s, 1H), 4.30 (d, J=6.3 Hz, 1H), 4.25 (d, J=5.5 Hz, 1H), 4.19-4.16 (m, 2H), 3.83-3.79 (m, 4H), 3.40-3.30 (m, 4H), 2.47 (s, 3H), 2.14-2.09 (m, 1H), 2.05 (dd, J=11.7, 4.1 Hz, 2H), 1.97-1.92 (m, 1H), 1.86 (d, J=10.9 Hz, 2H), 1.51 (d, J=12.1 Hz, 2H), 1.42-1.28 (m, 8H), 1.21 (dd, J=8.3, 3.6 Hz, 4H), 0.96 (s, 9H).

### Example A254. 2-amino-3-ethynyl-N-((S)-6-(6-(4-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-1-yl)methyl)piperidine-1-carbonyl)pyridin-3-yl)-2,3-dihydrobenzofuran-3-yl)-N-methylquinoline-6-carboxamide

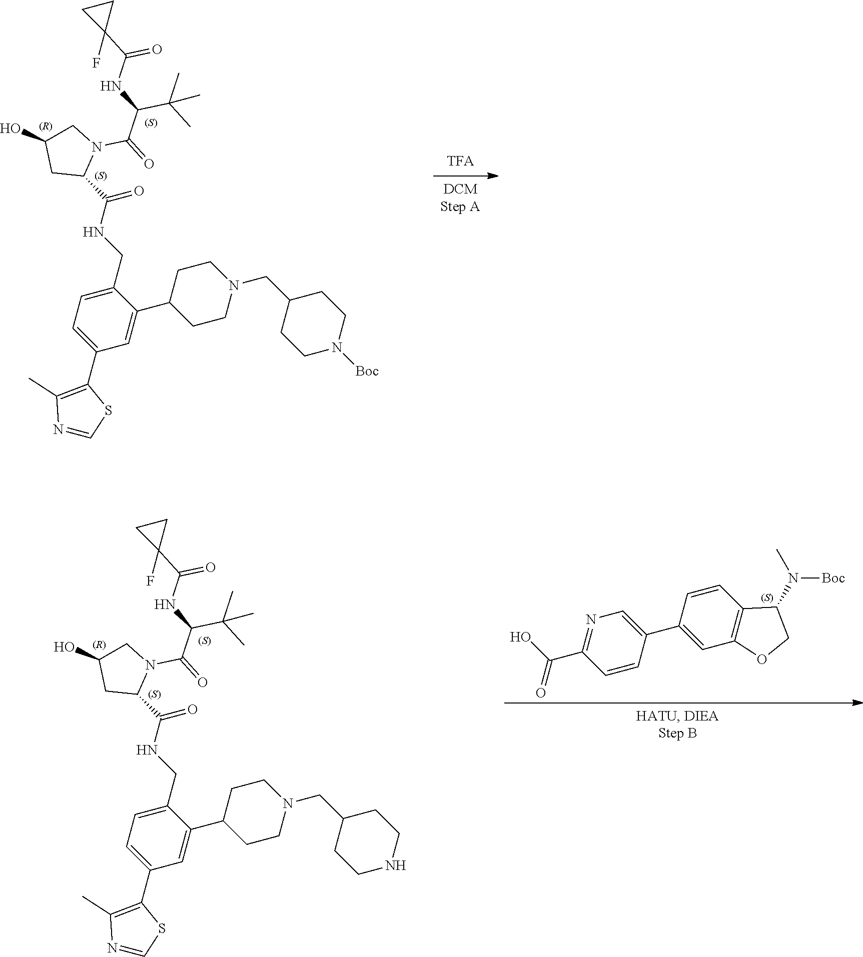

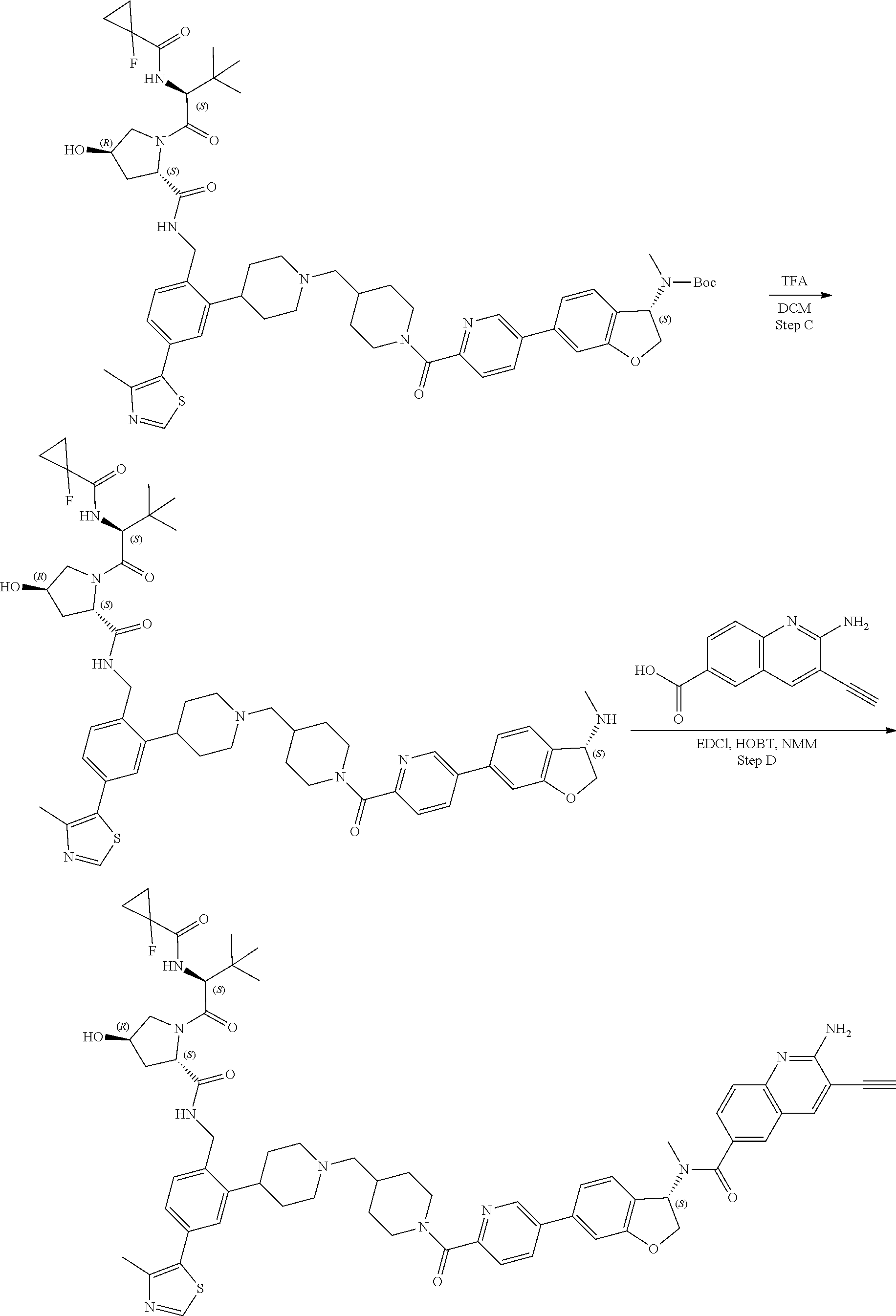

Example A254 was synthesized following the same procedures as described in Example A114 (5.3 mg, 7% yield over 4 steps) as a white solid. LC-MS (ESI): mass calcd. for C₆₄H₇₁FN₁₀O₇S, 1142.52; m/z found, 1143.9 [M+H]⁺. ¹H NMR (400 MHz, DMSO-d₆) δ 9.01 (s, 2H), 8.90 (s, 1H), 8.59 (s, 1H), 8.53 (s, 1H), 8.23 (dd, J=8.2, 2.0 Hz, 1H), 8.03 (s, 1H), 7.85 (d, J=8.6 Hz, 1H), 7.70 (s, 1H), 7.64 (d, J=8.1 Hz, 1H), 7.55 (d, J=7.5 Hz, 1H), 7.48 (d, J=8.1 Hz, 1H), 7.39 (d, J=7.8 Hz, 1H), 7.35-7.25 (m, 3H), 7.23 (s, 1H), 4.88 (s, 1H), 4.85-4.73 (m, 1H), 4.71-4.66 (m, 1H), 4.63-4.54 (m, 2H), 4.52-4.45 (m, 2H), 4.39-4.31 (m, 2H), 3.86-3.80 (m, 4H), 3.23-3.09 (m, 4H), 3.08-2.99 (m, 3H), 2.91-2.84 (m, 1H), 2.67 (s, 3H), 2.47 (s, 3H), 2.23-2.15 (m, 1H), 2.10-1.95 (m, 5H), 1.94-1.85 (m, 2H), 1.78-1.71 (m, 1H), 1.46-1.14 (m, 7H), 0.98 (s, 9H).

### Example A255. 2-amino-3-(difluoromethyl)-N-((S)-6-(6-(((18,4S)-4-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)

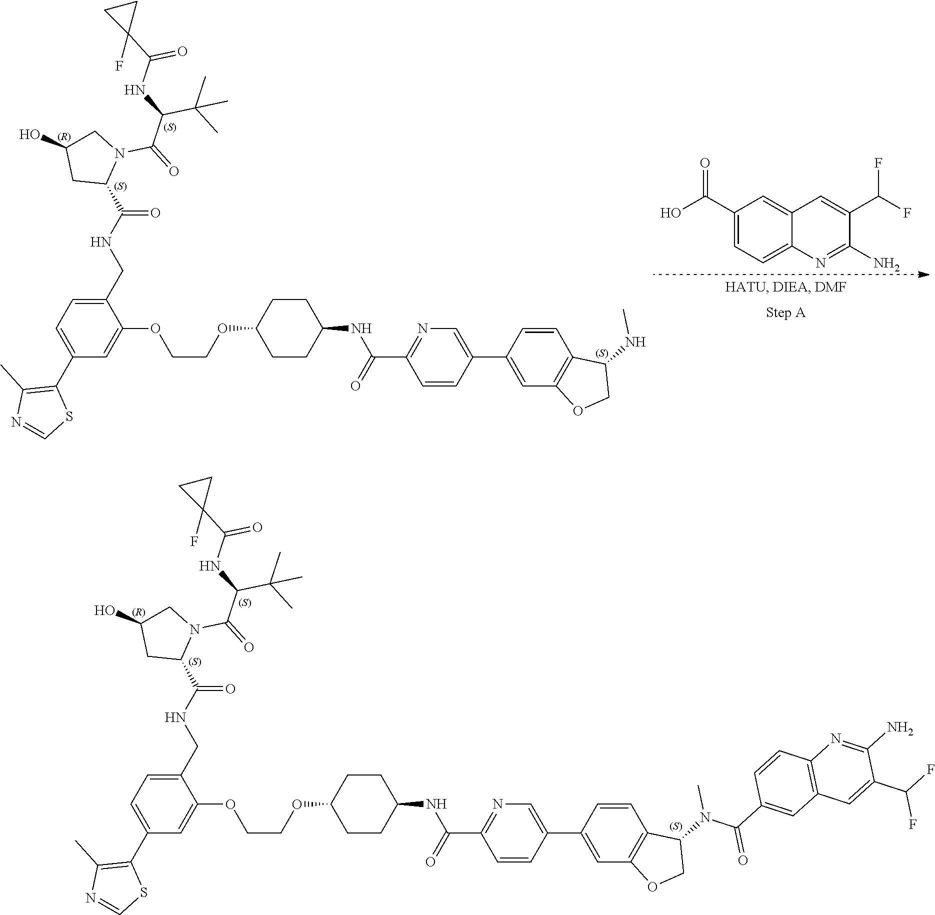

Example A255 was synthesized following the same procedures as described in Example A248 (1.5 mg, 1% yield over 6 steps) as a white solid. LC-MS (ESI): mass calcd. for C₆₀H₆₆F₃N₉O₉S, 1145.5/z found, 1146.5 [M+H]⁺. ¹H NMR (400 MHz, DMSO-d₆)) δ 8.99 (s, 1H), 8.92 (s, 1H), 8.57-8.48 (m, 3H), 8.28 (d, J=10.2 Hz, 1H), 8.14-8.04 (m, 2H), 7.84 (d, J=8.8 Hz, 1H), 7.69 (s, 1H), 7.57 (d, J=7.8 Hz, 1H), 7.41 (d, J=7.9 Hz, 2H), 7.36-7.26 (m, 3H), 7.21 (s, 1H), 7.05 (s, 1H), 6.97 (d, J=8.8 Hz, 1H), 5.32 (t, J=4.6 Hz, 1H), 4.71-4.66 (m, 1H), 4.63-4.53 (m, 1H), 4.52 (t, J=8.2 Hz, 1H), 4.41-4.23 (m, 3H), 4.23-4.12 (m, 2H), 3.88-3.72 (m, 4H), 3.68-3.55 (m, 4H), 2.68 (s, 3H), 2.47 (s, 3H), 2.04-1.92 (m, 4H), 1.92-1.82 (m, 2H), 1.53-1.42 (m, 4H), 1.40-1.29 (m, 4H), 0.96 (s, 9H).

### Example A256. 4-amino-3-cyclopropyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-1H-pyrazolo[4,3-c]quinoline-8-carboxamide

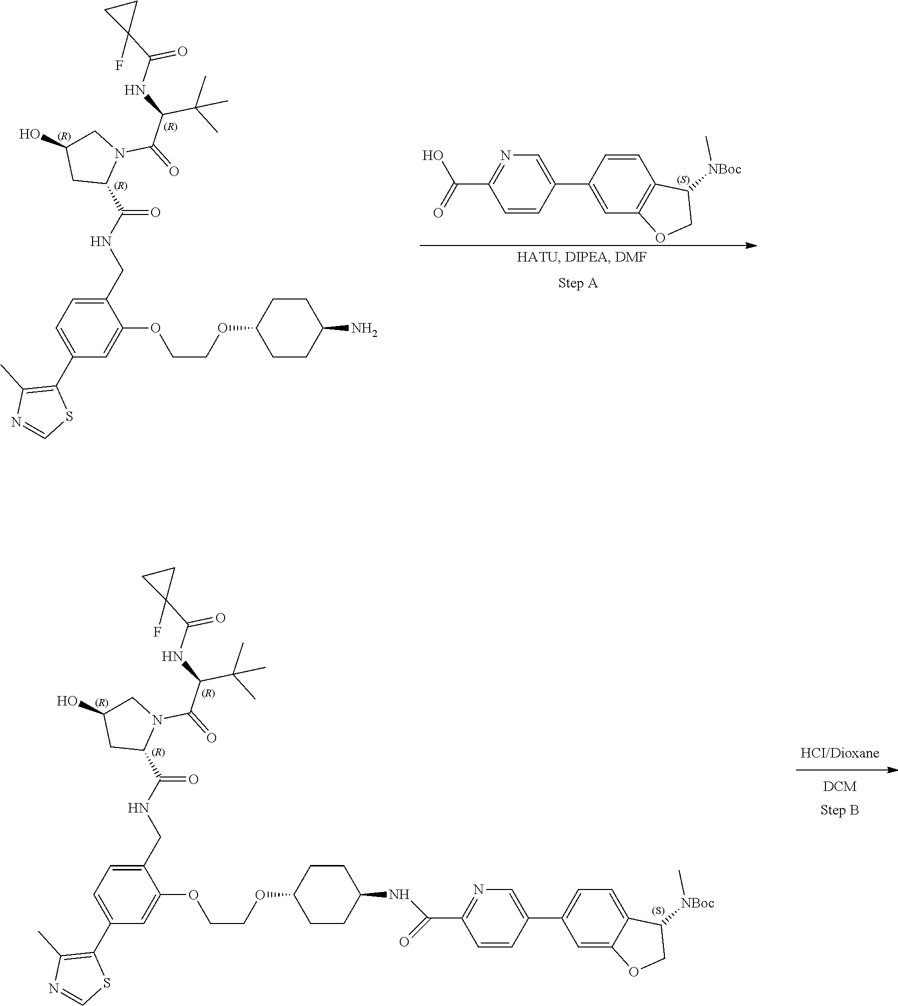

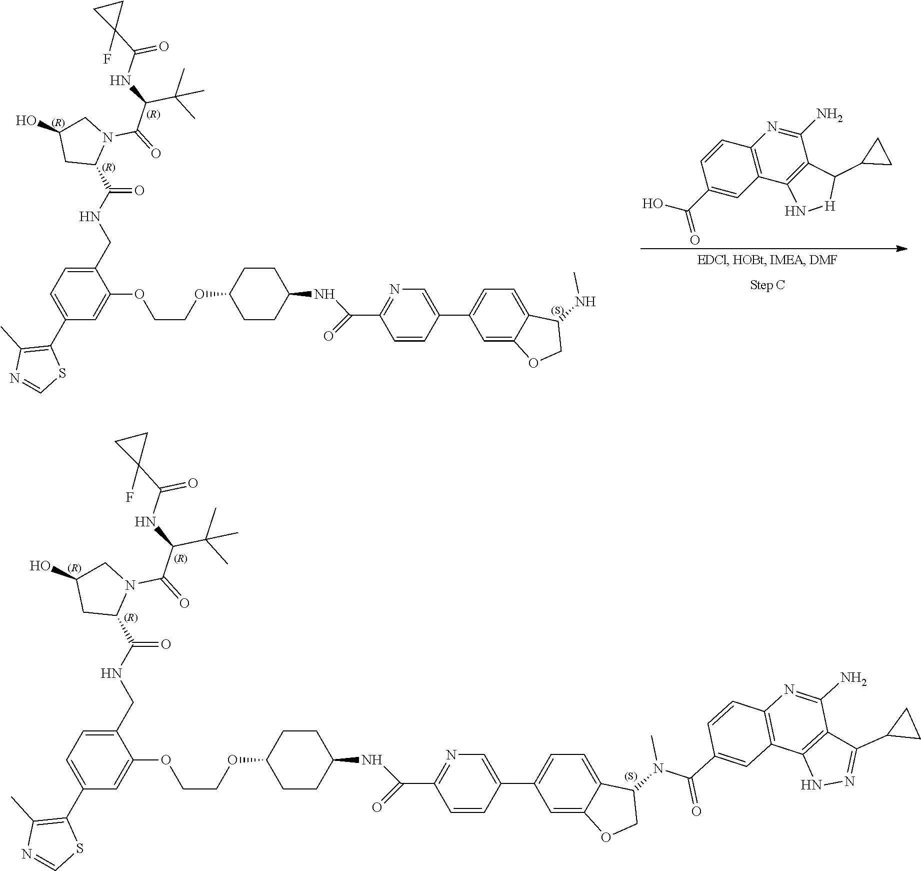

Example A256 was synthesized following the same procedures as described in Example A114 and A188 (2 mg, 3.5% yield over 3 steps) as a white solid. LC-MS (ESI): mass calcd. for C₆₃H₇₀FN109S, 1175.51; m/z found, 1176.5 [M+H]⁺. ¹H NMR (400 MHz, DMSO-d₆) δ 8.99 (s, 1H), 8.92 (s, 1H), 8.75 (s, 1H), 8.48 (d, J=9.2 Hz, 2H), 8.39 (s, 1H), 8.27 (d, J=7.6 Hz, 1H), 8.09 (d, J=8.2 Hz, 1H), 7.86-7.82 (m, 2H), 7.56 (s, 1H), 7.43-7.33 (m, 3H), 7.29 (d, J=6.7 Hz, 1H), 7.05 (s, 1H), 6.97 (d, J=7.7 Hz, 1H), 4.92-4.63 (m, 2H), 4.60 (d, J=9.0 Hz, 1H), 4.52 (t, J=8.2 Hz, 1H), 4.37-4.34 (m, 1H), 4.33-4.20 (m, 2H), 4.18 (s, 2H), 3.83-3.78 (m, 3H), 3.69-3.59 (m, 4H), 2.70 (s, 3H), 2.60-2.54 (m, 1H), 2.47 (s, 3H), 2.07-2.01 (m, 4H), 1.97-1.91 (m, 1H), 1.87-1.84 (m, 1H), 1.56-1.47 (m, 2H), 1.40-1.29 (m, 4H), 1.24-1.20 (m, 2H), 1.17-1.09 (m, 2H), 1.05-0.98 (m, 2H), 0.96 (s, 9H).

### Example A257. 2-amino-3-ethynyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)-5-methylpyridin-3-yl)-2,3-dihydrobenzofuran-3-yl)-N-methylquinoline-6-carboxamide

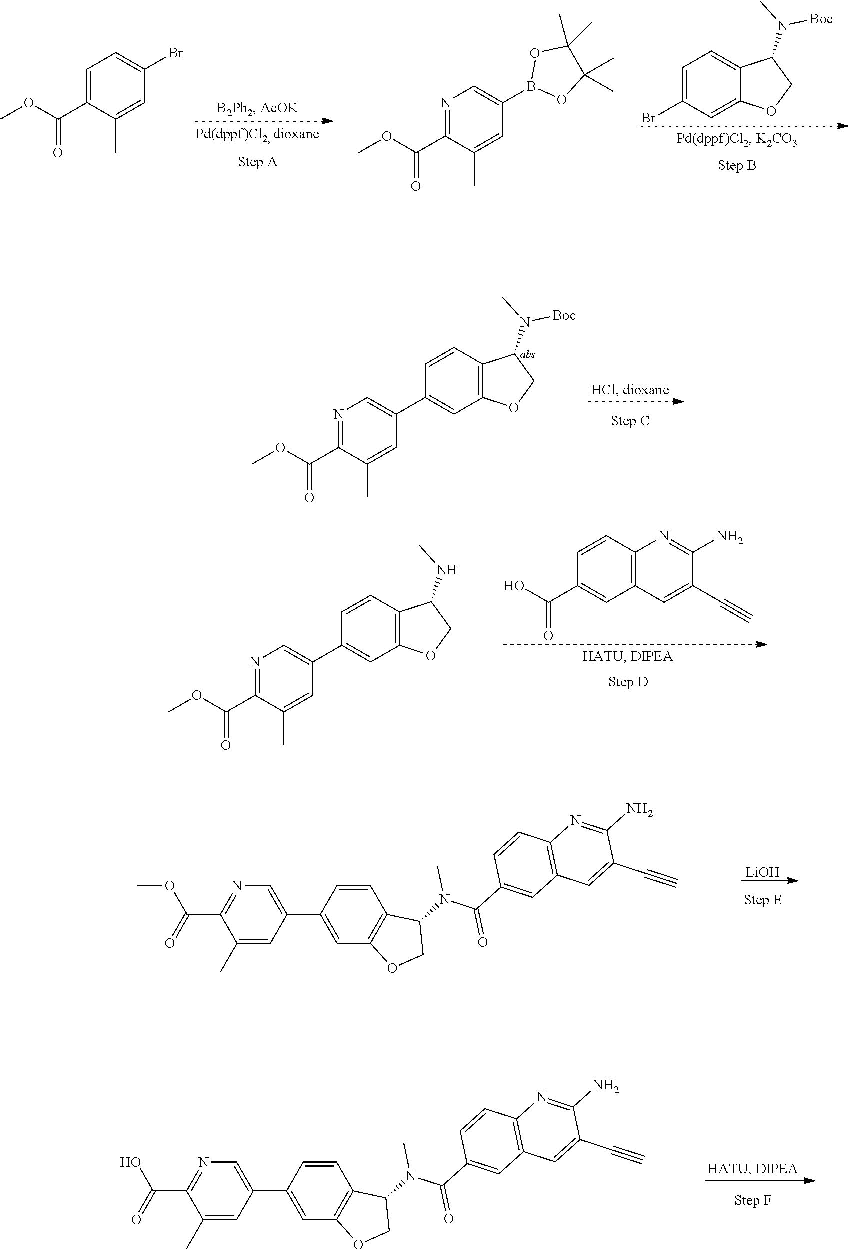

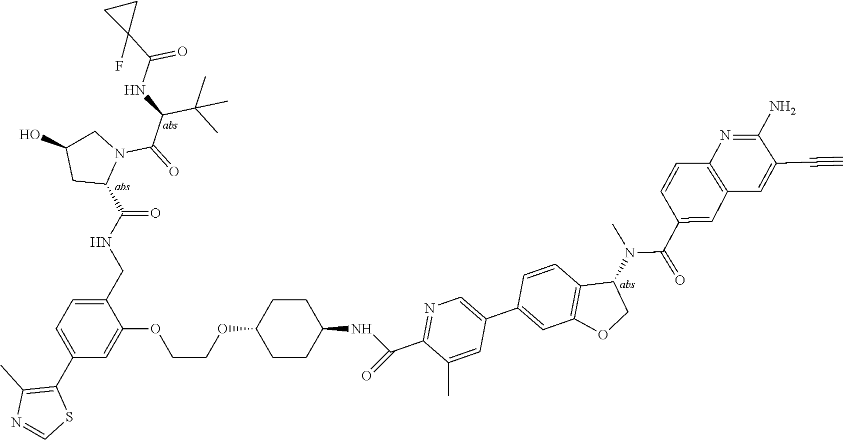 [SMILES: C#Cc1cc2cc(C(=O)N(C)[C@@H]3COc4cc(-c5cnc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)c(C)c5)ccc43)ccc2nc1N]

Example A257 was synthesized following the same procedures as described in Intermediate 3 and Example A9 (2.5 mg, 2% yield over 6 steps) as a white solid. LC-MS (ESI): mass calcd, for C₆₂H₆₈FN₉O₉S, 1133.5/z found, 1134.5 [M+H]⁺. ¹H NMR (400 MHz, DMSO-d₆)) δ 8.99 (s, 1H), 8.73 (s, 1H), 8.60 (s, 1H), 8.49 (s, 1H), 8.37 (d, J=8.1 Hz, 1H), 8.04 (s, 2H), 7.85 (d, J=9.8 Hz, 1H), 7.71 (s, 1H), 7.54 (d, J=7.7 Hz, 1H), 7.41 (d, J=7.6 Hz, 2H), 7.30 (d, J=14.6 Hz, 2H), 7.05 (s, 1H), 6.97 (d, J=7.7 Hz, 1H), 5.32 (t, J=4.6 Hz, 1H), 4.93-4.85 (m, 1H), 4.74-4.64 (m, 1H), 4.64-4.56 (m, 1H), 4.52 (t, J=8.3 Hz, 1H), 4.40-4.22 (m, 3H), 4.21-4.10 (m, 2H), 3.85-3.71 (m, 2H), 3.68-3.56 (m, 6H), 2.67 (s, 3H), 2.58 (s, 3H), 2.47 (s, 3H), 2.03-1.92 (m, 4H), 1.90-1.81 (m, 2H), 1.54-1.36 (m, 4H), 1.36-1.29 (m, 4H), 0.96 (s, 9H).

### Example A259. 2-amino-3-ethynyl-N-((S)-6-(5-fluoro-6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)

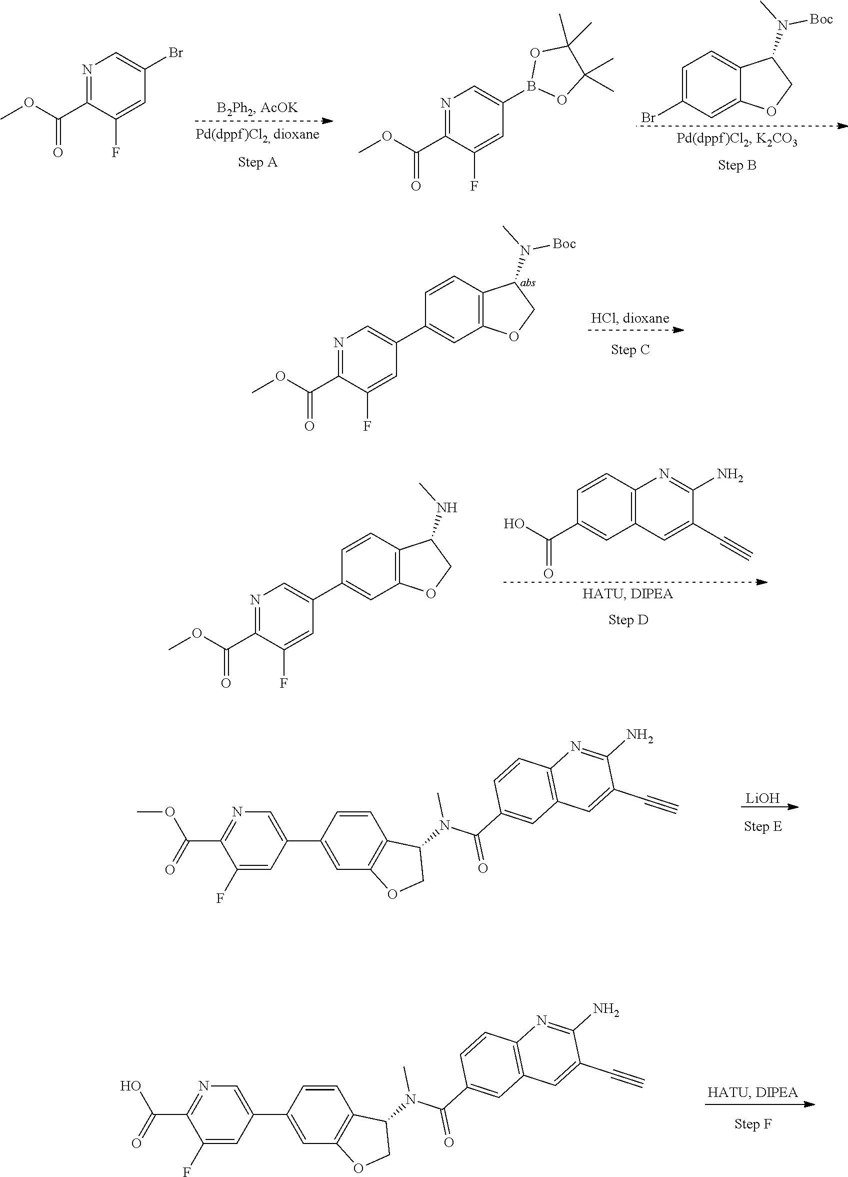

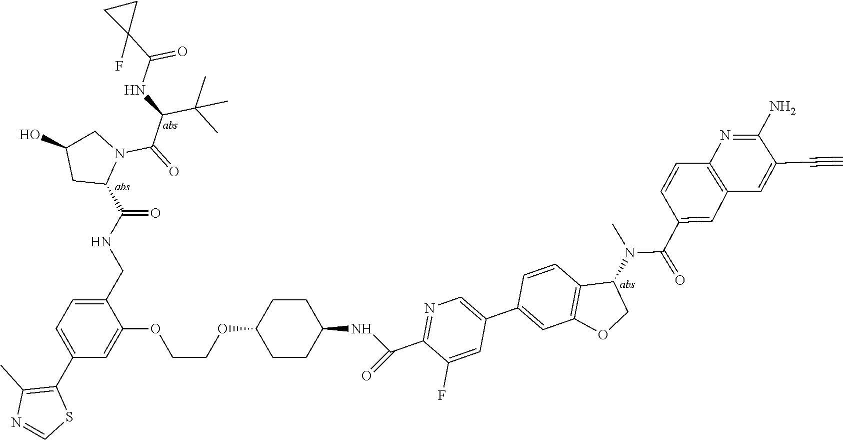 [SMILES: C#Cc1cc2cc(C(=O)N(C)[C@@H]3COc4cc(-c5cnc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)c(F)c5)ccc43)ccc2nc1N]

Example A259 was synthesized following the same procedures as described in Intermediate 3 and Example A9 (3.2 mg, 1% yield over 6 steps) as a white solid. LC-MS (ESI): mass calcd, for C₆₁H₆₅F₂N₉O₉S, 1137.5/z found, 1138.4 [M+H]⁺. ¹H NMR (400 MHz, DMSO-d₆) δ 8.99 (s, 1H), 8.80 (s, 1H), 8.64 (s, 1H), 8.52-8.48 (m, 1H), 8.46 (d,)=8.2 Hz, 1H), 8.20 (dd, J=11.9, 1.5 Hz, 1H), 8.05 (s, 1H), 7.88 (d, J=8.3 Hz, 1H), 7.75-7.69 (m, 1H), 7.56 (d, J=7.8 Hz, 1H), 7.45 (d, J=8.0 Hz, 1H), 7.41 (d, J=7.6 Hz, 2H), 7.29 (dd, J=9.2, 2.6 Hz, 1H), 7.05 (d, J=1.3 Hz, 1H), 6.99-6.96 (m, 1H), 4.95-4.91 (m, 1H), 4.80 (s, 1H), 4.71-4.67 (m, 1H), 4.60 (d, J=9.0 Hz, 1H), 4.52 (t, J=8.2 Hz, 1H), 4.40-4.32 (m, 2H), 4.30 (d, J=6.0 Hz, 1H), 4.24 (d, J=5.5 Hz, 1H), 4.21-4.15 (m, 3H), 3.40-3.31 (m, 2H), 2.66 (s, 3H), 2.47 (s, 3H), 2.08-2.03 (m, 2H), 1.99-1.93 (m, 1H), 1.90-1.84 (m, 2H), 1.50-1.35 (m, 4H), 1.34-1.21 (m, 6H), 0.95 (s, 9H)

### Example A260. N1-(3-amino-1-methyl-1H-pyrazolo[4,3-c]pyridin-7-yl)-N2-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N2-methyloxalamide

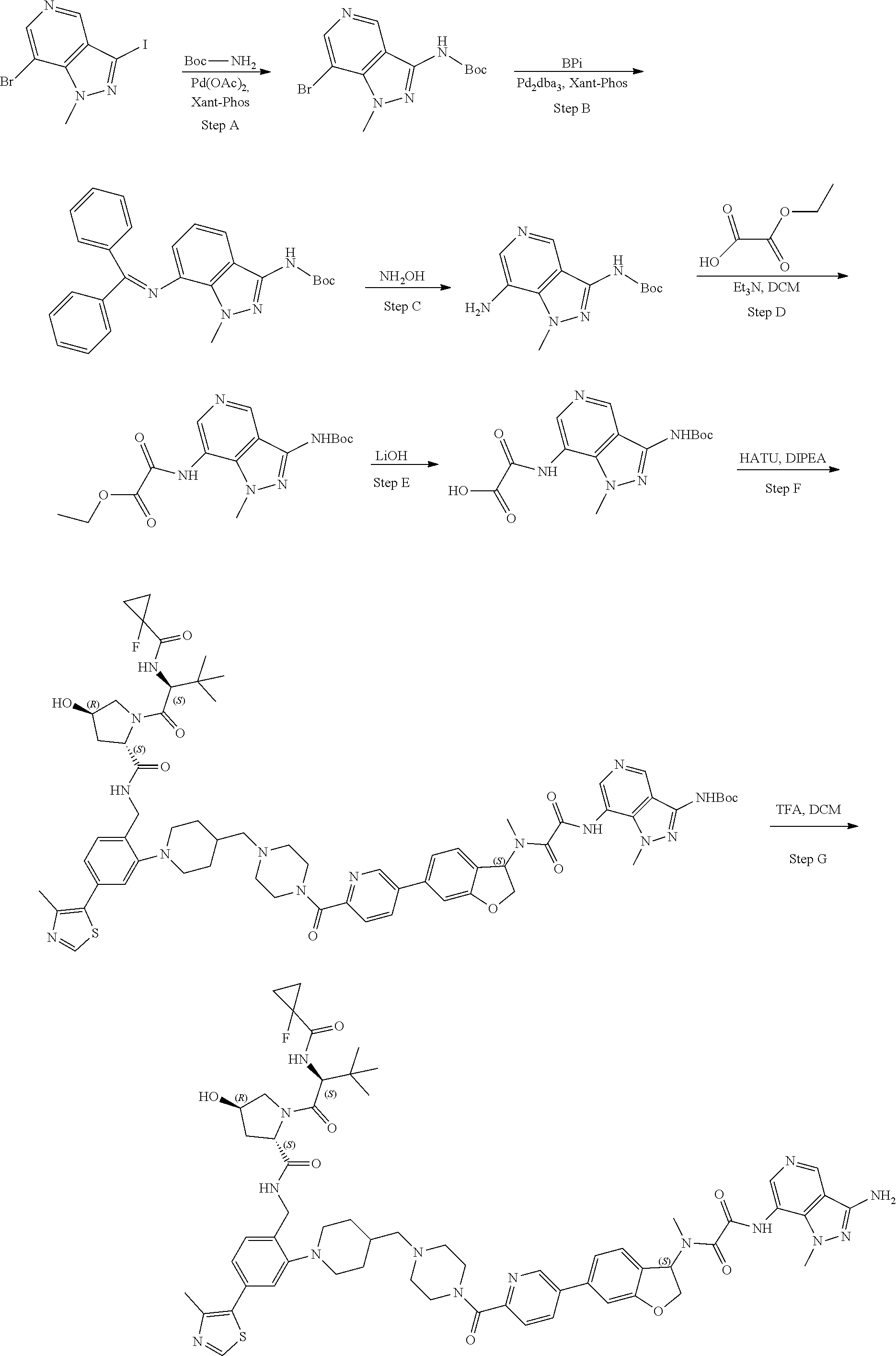

Step A: tert-butyl (7-bromo-1-methyl-1H-pyrazolo[4,3-c]pyridin-3-yl)carbamate

To a mixture of 7-bromo-3-iodo-1-methyl-1H-pyrazolo[4,3-c]pyridine (150 mg, 444 μmol, 1.0 eq.), tert-butyl carbamate (78 mg, 666 μmol, 1.5 eq.), xantphos (25.7 mg, 44.4 μmol, 0.1 eq.) and Pd (OAc) 2 (10.0 mg, 44.4 μmol, 0.1 eq.) in 1,4-Dioxane (5 mL) was added Cs₂CO₃ (434 mg, 1.3 mmol, 3.0 eq.). The reaction mixture was stirred at 80° C. for 12 hours. The reaction mixture was poured into water (20 mL) and then extracted with EtOAc (20 mL×2). The combined organic layers were washed with brine, dried over anhydrous Na₂SO₄ and concentrated. The residue was purified by column chromatography (100-200 mesh silica gel, 0%~60% EtOAc in PE) to afford tert-butyl (7-bromo-1-methyl-1H-pyrazolo[4,3-c]pyridin-3-yl)carbamate (30.0 mg, 20% yield) as a solid. LC-MS (ESI): mass calcd. For C₁₂H₁₅BrN₄O₂, 326.04; m/z found, 327.1 [M+H]⁺.

Step B: tert-butyl (7-((diphenylmethylene)amino)-1-methyl-1H-pyrazolo[4,3-c]pyridin-3-yl)carbamate

To a mixture of tert-butyl (7-bromo-1-methyl-1H-pyrazolo[4,3-c]pyridin-3-yl)carbamate (100 mg, 1.0 eq, 306 μmol) in 1,4-dioxane (5 mL) was added 4,5-Bis(diphenylphosphino)-9,9-dimethyl xanthene (17.7 mg, 0.1 eq., 30.6 μmol), palladium (II)acetate (6.86 mg, 0.1 eq., 30.6 μmol), benzophenone imine (83.1 mg, 77 μL, 1.5 eq., 458 μmol) and Cs₂CO₃ (299 mg, 3.0 eq., 917 μmol). The reaction mixture was stirred at 100° C. for 2 hours under N₂. The resulting mixture was filtered and concentrated. The crude product was purified by column chromatography (100-200 mesh silica gel, 0-50% EtOAc in PE) to afford tert-butyl (7-((diphenylmethylene)amino)-1-methyl-1H-pyrazolo[4,3-c]pyridin-3-yl)carbamate as a white solid (80 mg, 61.2% yield)

Step C: tert-butyl (7-amino-1-methyl-1H-pyrazolo[4,3-c]pyridin-3-yl)carbamate

A mixture of tert-butyl (7-((diphenylmethylene)amino)-1-methyl-1H-pyrazolo[4,3-c]pyridin-3-yl)carbamate (60.0 mg, 1 Eq, 140 μmol), sodium acetate (23.0 mg, 15.1 μL, 2 eq, 281 μmol) in MeOH (5 mL) were added hydroxylamine (6.95 mg, 1.5 eq, 211 μmol). The reaction mixture was purged with nitrogen three times, then heated to 80° C. for 12 hours under nitrogen. The resulting solution was diluted with 30 mL of water and extracted with EtOAc (3×30 mL). The combined organic layers were washed with brine, dried over anhydrous Na₂SO₄ and concentrated under reduced pressure. The resulting mixture was purified by prep-HPLC to afford tert-butyl (7-amino-1-methyl-1H-pyrazolo[4,3-c]pyridin-3-yl)carbamate (20 mg, 51.4% yield).

Steps D-G: N1-(3-amino-1-methyl-1H-pyrazolo[4,3-c]pyridin-7-yl)-N2-((S)-6-(6-(4-((1-(2-(((2S, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N2-methyloxalamide

Example A260 was synthesized following the same procedures as described in Example A31 (1.0 mg, 1% yield over 6 steps) as a white solid. LC-MS (ESI): mass calcd. for C₆₀H₇₁FN14O₈S, 1166.53; m/z found, 1168.0 [M+H]⁺. ¹H NMR (400 MHz, DMSO-d₆) δ 11.26 (d, J=31.1 Hz, 1H), 9.58 (s, 1H), 9.19 (s, 1H), 9.08-8.85 (m, 1H), 8.50 (d, J=13.3 Hz, 1H), 8.42-8.14 (m, 2H), 7.79 (d, J=7.7 Hz, 1H), 7.56 (s, 1H), 7.41 (d, J=14.7 Hz, 1H), 7.34-7.21 (m, 3H), 7.11 (d, J=13.8 Hz, 4H), 6.69 (s, 1H), 4.87-4.66 (m, 2H), 4.64-4.54 (m, 2H), 4.54-4.42 (m, 1H), 4.36 (s, 3H), 4.26-4.10 (m, 1H), 4.05-3.98 (m, 2H), 3.97-3.88 (m, 1H), 3.72-3.60 (m, 5H), 3.22-3.01 (m, 8H), 2.90-2.78 (m, 1H), 2.78-2.70 (m, 2H), 2.67 (s, 3H), 2.47-2.45 (m, 3H), 2.34-2.31 (m, 1H), 2.15-2.06 (m, 1H), 2.03-1.82 (m, 5H), 1.58-1.32 (m, 5H), 1.30-1.16 (m, 3H), 0.98 (s, 9H).

### Example A262. 2-amino-3-ethynyl-N-((S)-6-(3-fluoro-4-(((18,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)phenyl)-2,3-dihydrobenzofuran-3-yl)-N-methylquinoline-6-carboxamide

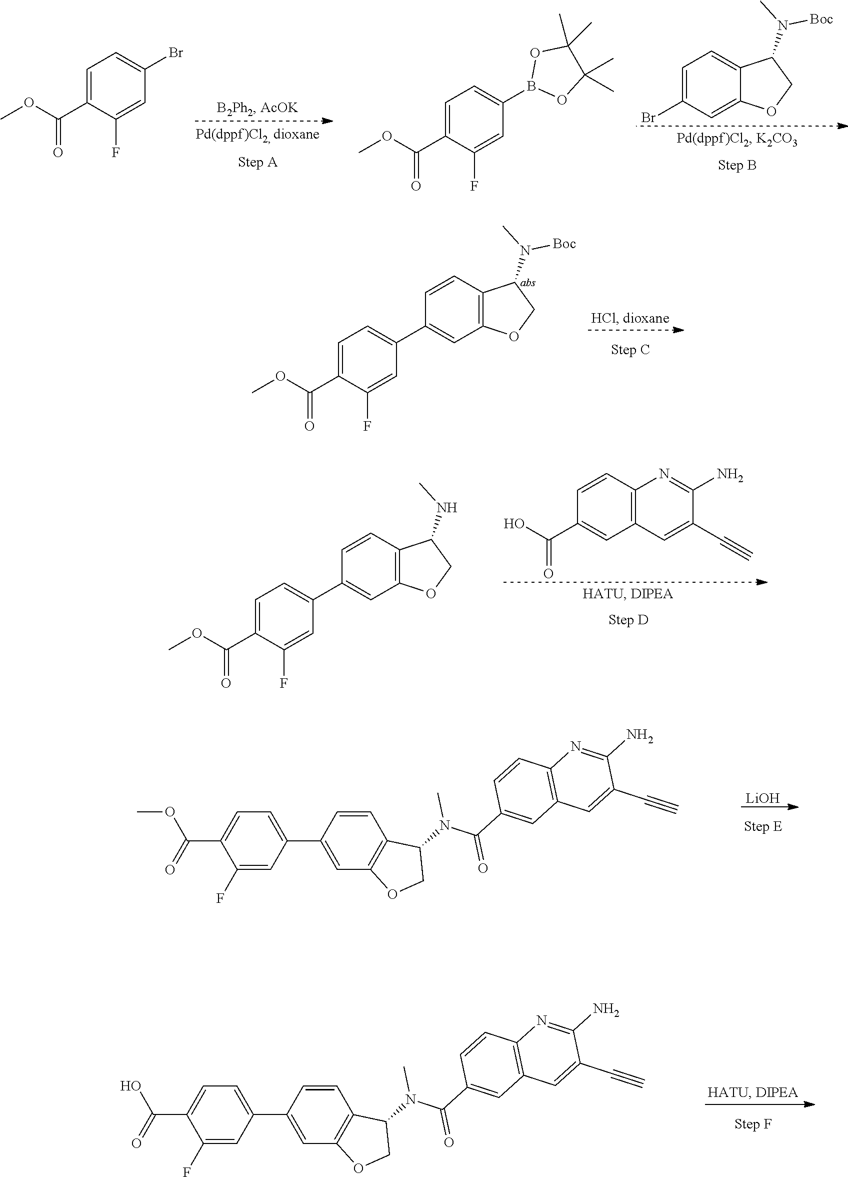

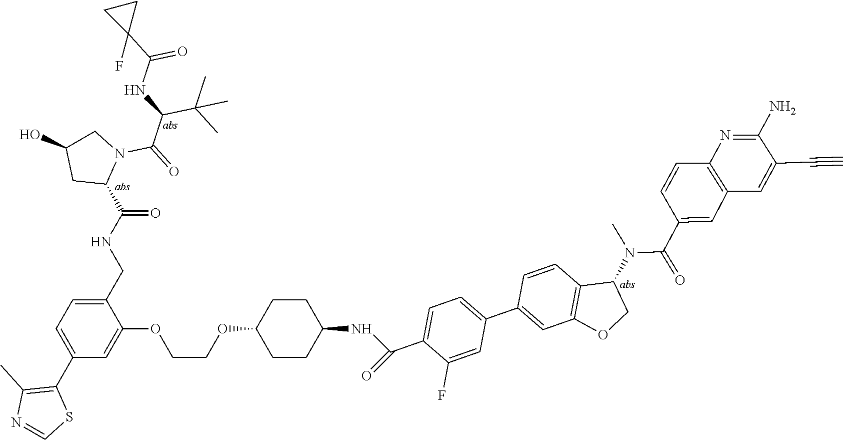 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)c(F)c5)ccc43)ccc2nc1N]

Example A262 was synthesized following the same procedures as described in Intermediate 3 and Example A9 (3.5 mg, 3% yield over 6 steps) as a white solid. LC-MS (ESI): mass calcd, for C₆₂H₆₆F₂N₈O₉S, 1136.46; m/z found, 1137 [M+H]⁺. ¹H NMR (400 MHz, DMSO-d₆) δ 8.99 (s, 1H), 8.67 (s, 1H), 8.49 (t, J=5.8 Hz, 2H), 8.18 (d, J=7.5 Hz, 1H), 8.06 (s, 1H), 7.89 (d, J=8.6 Hz, 1H), 7.74 (s, 1H), 7.64-7.57 (m, 3H), 7.51 (d, J=7.8 Hz, 1H), 7.41 (d, J=7.8 Hz, 1H), 7.35 (d, J=7.4 Hz, 1H), 7.29 (d, J=6.8 Hz, 2H), 7.05 (s, 1H), 6.97 (d, J=7.8 Hz, 1H), 4.94 (s, 1H), 4.79 (s, 1H), 4.69 (s, 1H), 4.60 (d,)=9.2 Hz, 1H), 4.51 (d, J=8.3 Hz, 1H), 4.34 (d, J=12.6 Hz, 2H), 4.29 (d, J=6.1 Hz, 1H), 4.24 (d, J=5.7 Hz, 1H), 4.17 (s, 2H), 3.80 (s, 1H), 3.75-3.74 (m, 1H), 3.65 (s, 1H), 3.61-3.60 (m, 1H), 3.35 (s, 1H), 2.66 (s, 3H), 2.46 (s, 3H), 2.13-2.07 (m, 1H), 2.06-2.00 (m, 2H), 1.98-1.92 (m, 1H), 1.92-1.85 (m, 2H), 1.41-1.36 (m, 2H), 1.35-1.33 (m, 2H), 1.32-1.26 (m, 2H), 1.24-1.20 (m, 2H), 0.96 (s, 9H).

### Example A263. 2-amino-3-cyano-N-((3S)-6-(6-(((18,4S)-4-(2-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl) pyrrolidin-1-yl)cyclohexyl) carbamoyl)pyridin-3-yl)-2,3-dihydrobenzofuran-3-yl)-N-methylquinoline-6-carboxamide

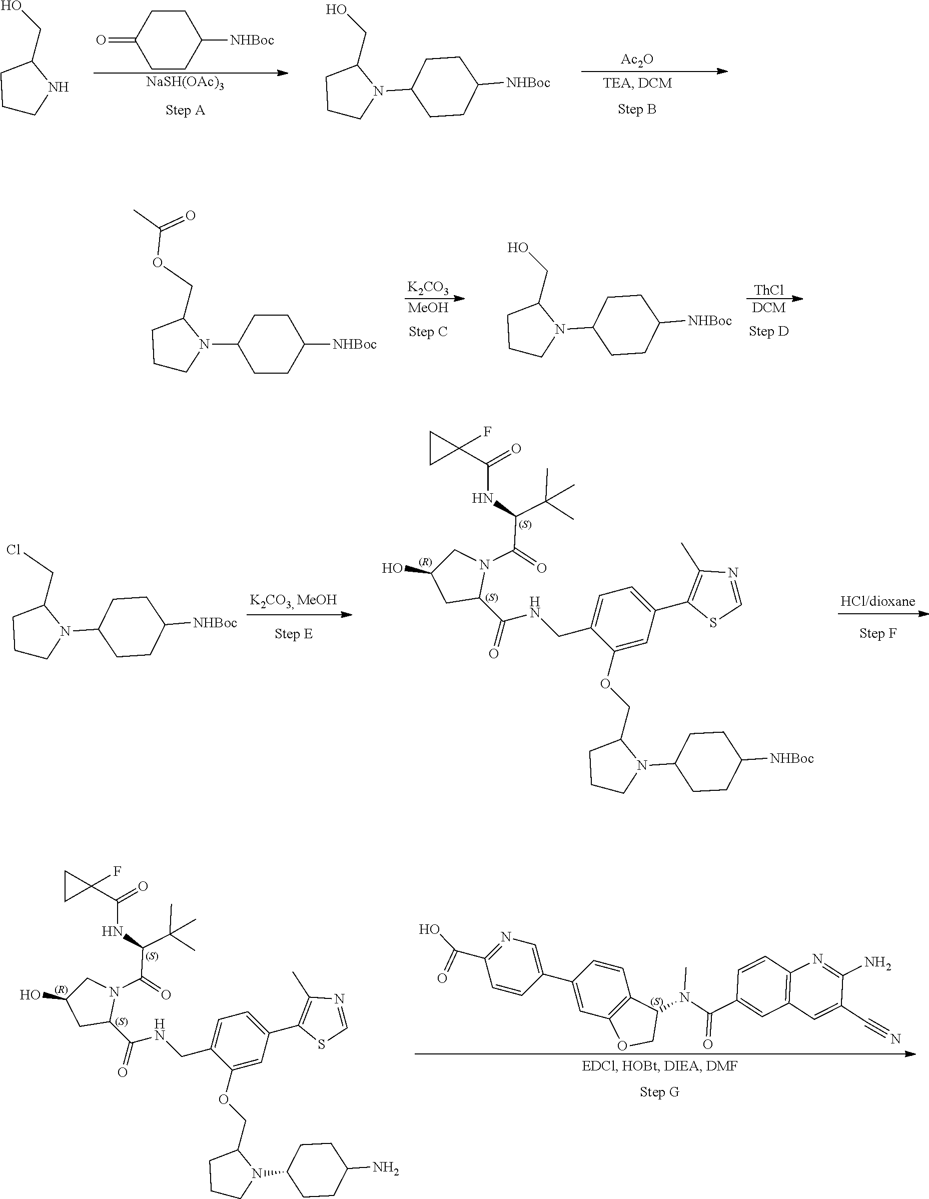

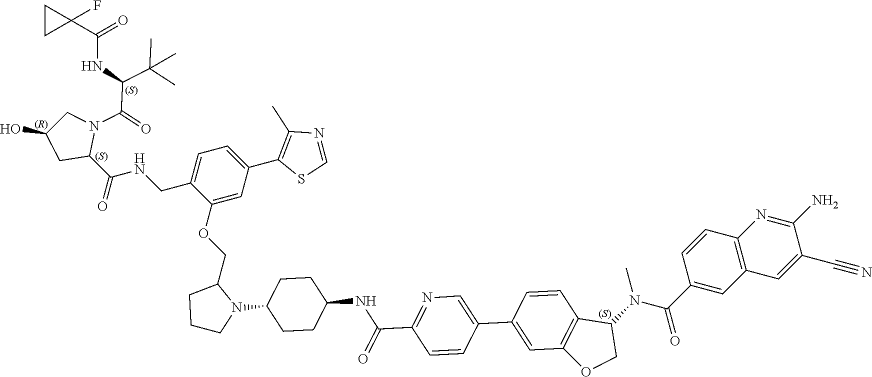

Step A: tert-butyl (4-(2-(hydroxymethyl) pyrrolidin-1-yl)cyclohexyl)carbamate

To a solution of pyrrolidin-2-ylmethanol (500 mg, 4.94 mmol, 1.0 eq.) in DMF (10 mL) was added tert-butyl (4-oxocyclohexyl)carbamate (1.05 g, 4.94 mmol, 1.0 eq.). The mixture was stirred for 0.5 hours. then Sodium triacetoxyborohydride (2.1 g, 9.89 mmol, 2.0 eq.) was added. let the reaction mixture stirred for additional 4 hours. The reaction mixture was evaporated to afford tert-butyl (4-(2-(hydroxymethyl) pyrrolidin-1-yl)cyclohexyl)carbamate (500 mg, 34% yield) as an oil, which was used directly without further purification. LC-MS (ESI): mass calcd. For C₁₆H₃₀N₂O₃, 298.23; m/z found, 299.3 [M+H]⁺.

Step B: (1-(4-((tert-butoxycarbonyl)amino)cyclohexyl) pyrrolidin-2-yl) methyl acetate

To a stirred mixture of tert-butyl (4-(2-(hydroxymethyl) pyrrolidin-1-yl)cyclohexyl)carbamate (100 mg, 335 μmol, 1.0 eq.) in DCM (8 mL.) were added TEA (100 mg, 140 μL, 1.01 mmol, 3.0 eq.), acetic anhydride (51.0 mg, 47.4 μL, 503 μmol, 1.5 eq.). The mixture was stirred at room temperature for 2 hours. The reaction mixture was diluted with water (30 mL), extracted with EtOAc (30 mL×3), then the combined extracts were dried over anhydrous Na₁SO₄, filtered and evaporated. The crude product was purified by flash silica chromatography (eluting with a gradient of 0~10% MeOH in DCM) to afford (1-(4-((tert-butoxycarbonyl)amino)cyclohexyl) pyrrolidin-2-yl) methyl acetate (40 mg, 35% yield) as a solid.

LC-MS (ESI); mass calcd. For C₁₈H₃₂N₂O₄, 340.24; m/z found, 341.3 [M+H]⁺.

Step C: tert-butyl (4-(2-(hydroxymethyl) pyrrolidin-1-yl)cyclohexyl)carbamate

To a solution of (1-(4-((tert-butoxycarbonyl)amino)cyclohexyl) pyrrolidin-2-yl) methyl acetate (40.0 mg, 117 μmol, 1.0 eq.) in MeOH (3 mL) was added K₂CO₃ (50 mg, 352 umo, 3.0 eq.). The mixture was stirred at room temperature for 4 hours. The reaction mixture was filtered and concentrated under reduced pressure to give a residue, which was triturated with MeOH (10 mL), filtered and dried over vacuum to afford tert-butyl (4-(2-(hydroxymethyl) pyrrolidin-1-yl)cyclohexyl)carbamate (30.0 mg, 85% yield) as an yellow oil, which was used directly without further purification. LC-MS (ESI): mass calcd. For C₁₆H₃₀N₂O₃, 298.23; m/z found, 299.3 [M+H]⁺.

Step D: tert-butyl (4-(2-(chloromethyl) pyrrolidin-1-yl)cyclohexyl)carbamate

To a stirred mixture of tert-butyl (4-(2-(hydroxymethyl) pyrrolidin-1-yl)cyclohexyl)carbamate (30.0 mg, 101 μmol, 1.0 eq.) in DCM (3 mL) were added TEA (31 mg, 42.0 μL, 302 μmol, 3.0 eq.), 4-methylbenzenesulfonyl chloride (29 mg, 151 μmol, 1.5 eq.). DMAP (3 mg, 20.1 μmol, 0.4 eq.) was then added and the mixture was stirred at room temperature for 3 hours. The reaction mixture was diluted with water (30 mL), extracted with DCM (30 mL×3), then the combined extracts were dried over anhydrous Na₂SO₄, filtered and evaporated. The crude product was purified by flash silica chromatography, eluting with a gradient of 0~3% MeOH in DCM to afford tert-butyl (4-(2-(chloromethyl) pyrrolidin-1-yl)cyclohexyl)carbamate (20.0 mg, 63% yield) as a white solid. LC-MS (ESI): mass calcd. For C₁₆H₂₉ClN₂O₂, 316.19; m/z found, 317.2 [M+H]⁺.

Steps E-G: 2-amino-3-cyano-N-(35)-6-(6-(((1S,4S)-4-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl) pyrrolidin-1-yl)cyclohexyl) carbamoyl)pyridin-3-yl)-2,3-dihydrobenzofuran-3-yl)-N-methylquinoline-6-carboxamide

Example A263 was synthesized following the same procedures as described in Example A29 (2.3 mg, 6% yield over 3 steps) as a white solid. LC-MS (ESI): mass calcd. for C₆₃H₇FN₁₁O₈S, 1159.50/z found, 1161.0 [M+H]⁺. ¹H NMR (400 MHz, DMSO-d₆) δ 9.02-8.99 (m, 1H), 8.95-8.91 (m, 1H), 8.79 (s, 1H), 8.63-8.53 (m, 1H), 8.49-8.44 (m, 1H), 8.32-8.28 (m, 1H), 8.11-8.08 (m, 1H), 7.96 (s, 1H), 7.82-7.78 (m, 1H), 7.60-7.54 (m, 2H), 7.49-7.42 (m, 1H), 7.40-7.36 (m, 1H), 7.35-7.24 (m, 4H), 7.14-7.03 (m, 2H), 4.80-4.67 (m, 2H), 4.61-4.56 (m, 1H), 4.54-4.49 (m, 1H), 4.45-4.23 (m, 5H), 4.17 (s, 1H), 3.76-3.60 (m, 7H), 2.67 (s, 3H), 2.47-2.45 (m, 3H), 2.25-2.19 (m, 1H), 2.06-1.71 (m, 13H), 1.40-1.32 (m, 2H), 1.23-1.19 (m, 2H), 0.96-0.92 (m, 9H).

### Example A264. 2-amino-3-cyano-N-((S)-6-(6-(((S)-1-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)-4,5,6,7-tetrahydro-1H-indazol-5-yl)carbamoyl)pyridin-3-yl)-2,3-dihydrobenzofuran-3-yl)-N-methylquinoline-6-carboxamide

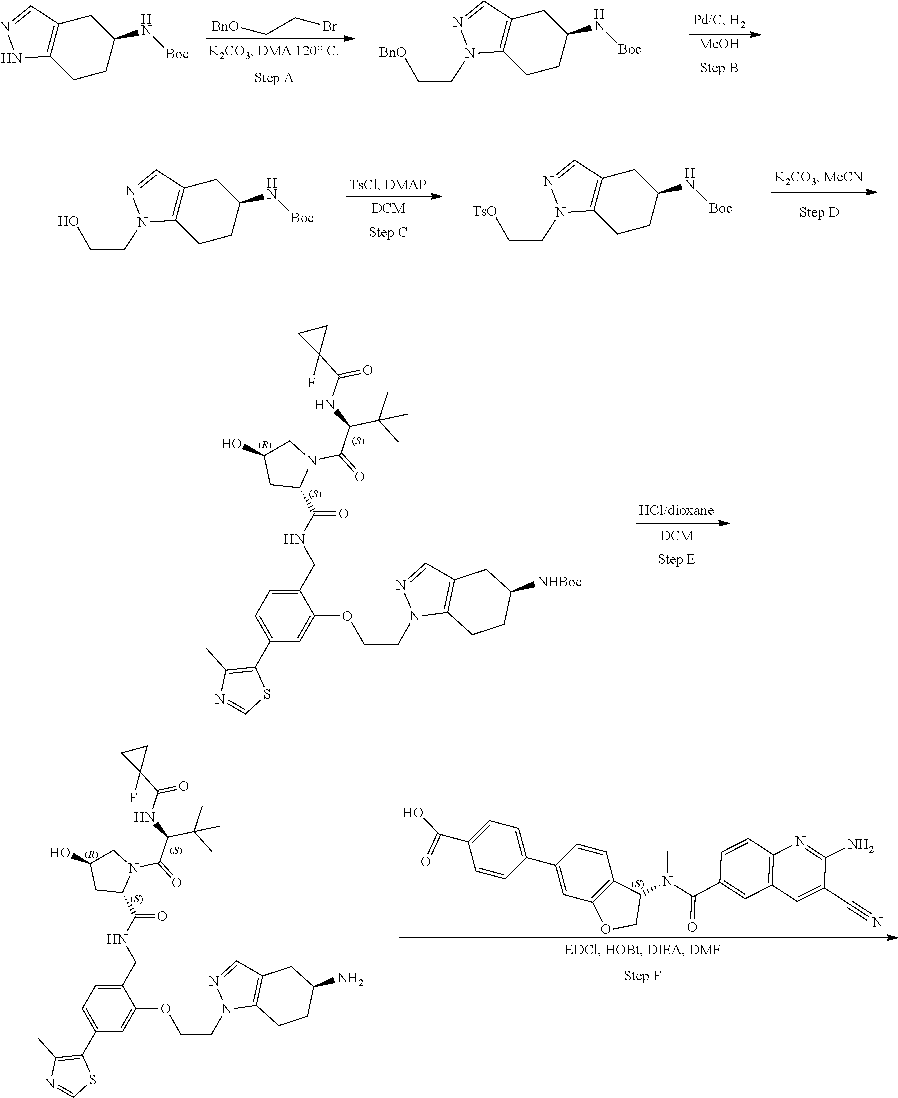

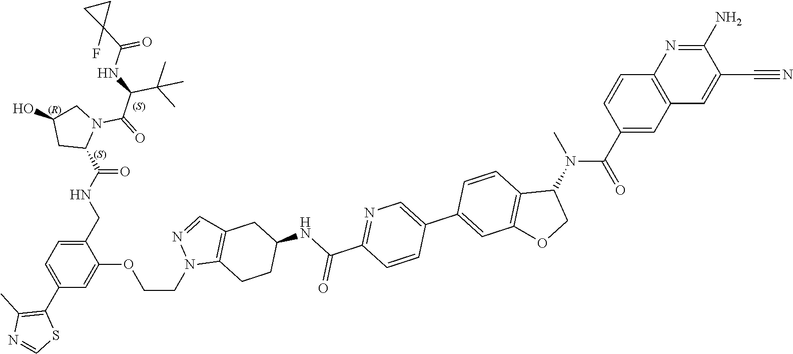

Step A: tert-butyl(S)-(1-(2-(benzyloxy)ethyl)-4,5,6,7-tetrahydro-1H-indazol-5-yl)carbamate

To a mixture of tert-butyl(S)-(4,5,6,7-tetrahydro-1H-indazol-5-yl)carbamate (300.0 mg, 1.264 mmol, 1.0 eq.), K₂CO₃ (873.6 mg, 6.321 mmol, 3.0 eq.), ((2-bromoethoxy)methyl) benzene (407.9 mg, 1.896 mmol, 1.5 eq.) and KI (104.9 mg, 632.1 μmol, 0.5 eq.) in DMA (5 mL) was stirred at 120° C. for 12 hour under N₂ atmosphere. The resulting solution was diluted with 10 mL of water and extracted with EtOAc (30 mL×3) The combined organic layers were washed with brine, dried over anhydrous Na₂SO₄ and concentrated under reduced pressure. The residue was purified by flash column chromatography on silica gel (silica gel, 0-100% EtOAc in PE) to afford tert-butyl(S)-(1-(2-(benzyloxy)ethyl)-4,5,6,7-tetrahydro-1H-indazol-5-yl)carbamate (82.0 mg, 17% yield) as a yellow solid. LC-MS (ESI): mass calcd. For C₂₁H₂₉N₃O₃, 371.22; m/z found, 372.22 [M+H]⁺.

Step B: tert-butyl(S)-(1-(2-hydroxyethyl)-4,5,6,7-tetrahydro-1H-indazol-5-yl)carbamate

To the solution of tert-butyl(S)-(1-(2-(benzyloxy)ethyl)-4,5,6,7-tetrahydro-1H-indazol-5-yl)carbamate (200.0 mg, 538.4 μmol, 1.0 eq.) in MeOH (5.00 mL) was added Pd/C (143.2 mg, 1.346 mmol, 2.5 eq.). The mixture was stirred at 50° C. for 24 hours under H₂ atmosphere. The resulting solution was filtered, concentrated and purified by flash column chromatography on silica gel (silica gel, 0-10% MeOH in DCM) to afford tert-butyl(S)-(1-(2-hydroxyethyl)-4,5,6,7-tetrahydro-1H-indazol-5-yl)carbamate (126 mg, 83% yield) as a yellow solid. LC-MS (ESI): mass calcd. For C₁₄H₂₃N₃O₃, 281.17; m/z found, 282.17 [M+H]⁺.

Steps C-F: 2-amino-3-cyano-N-(S)-6-(6-(((S)-1-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)-4,5,6,7-tetrahydro-1H-indazol-5-yl)carbamoyl)pyridin-3-yl)-2,3-dihydrobenzofuran-3-yl)-N-methylquinoline-6-carboxamide

Example A264 was synthesized following the same procedures as described in Example A29 (2.9 mg, 11% yield over 4 steps) as a white solid. LC-MS (ESI): mass calcd. For C₆₁H₆₃FN₁₂O₈S, 1142.46; m/z found, 1143.46 [M+H]⁺. ¹H NMR (400 MHz, DMSO-d₆) δ 9.00 (d, J=1.9 Hz, 1H), 8.95-8.82 (m, 2H), 8.63 (t, 1H), 8.50-8.47 (m, 1H), 8.29 (dd, J=8.2, 2.0 Hz, 1H), 8.11 (d, J=8.1 Hz, 1H), 8.00 (s, 1H), 7.86 (dd, J=8.7, 1.8 Hz, 1H), 7.75-7.62 (m, 2H), 7.56 (d, J=6.7 Hz, 2H), 7.50-7.22 (m, 5H), 6.97 (d, J=9.5 Hz, 2H), 4.71 (s, 1H), 4.86-4.66 (m, 2H), 4.59 (d, J=9.2 Hz, 1H), 4.52 (t, J=8.2 Hz, 1H), 4.46-4.38 (m, 3H), 4.35 (s, 3H), 4.27-4.20 (m, 2H), 3.69-3.55 (m, 3H), 2.88-2.73 (m, 2H), 2.67 (s, 3H), 2.67-2.58 (m, 2H), 2.45 (d, J=3.5 Hz, 3H), 2.12-2.06 (m, 1H), 2.01-1.87 (m, 3H), 1.40-1.31 (m, 2H), 1.23-1.18 (m, 2H), 0.94 (s, 9H).

### Example A265. 2-amino-3-ethynyl-N-((S)-6-(4-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)phenyl)-2,3-dihydrobenzofuran-3-yl)-N-methylquinoline-6-carboxamide

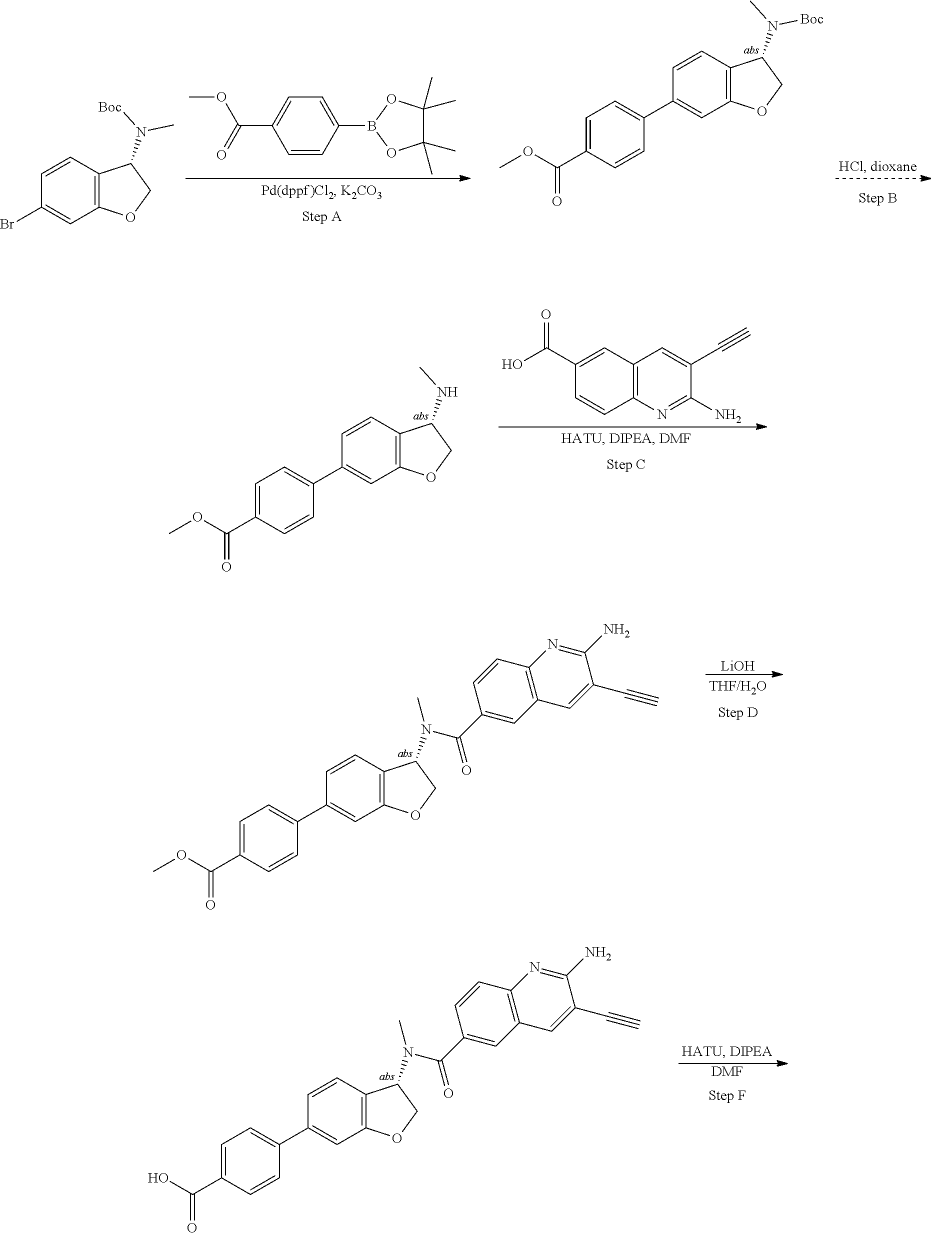

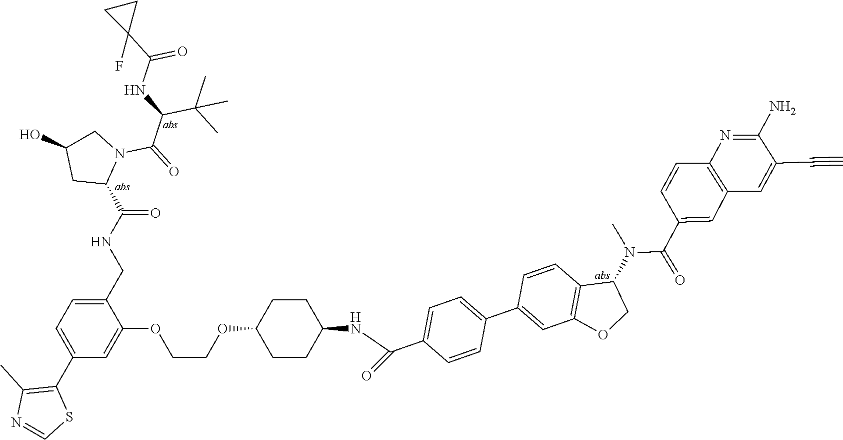 [SMILES: C#Cc1cc2cc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)cc5)ccc43)ccc2nc1N]

Example A265 was synthesized following the same procedures as described in Example A71 (3.3 mg, 7% yield over 5 steps) as a white solid. LC-MS (ESI): mass calcd, for C₆₂H₆₇FNsOgS, 1118.47; m/z found, 1119.6 [M+H]⁺. ¹H NMR (400 MHz, DMSO-d₆) δ 8.99 (s, 1H), 8.64 (s, 1H), 8.50 (t, J=6.0 Hz, 1H), 8.26 (d, J=7.8 Hz, 2H), 8.05 (s, 1H), 7.93-7.86 (m, 3H), 7.75 (d, J=8.3 Hz, 3H), 7.50 (d, J=7.7 Hz, 1H), 7.41 (d, J=7.8 Hz, 1H), 7.35-7.22 (m, 3H), 7.06 (d, J=1.3 Hz, 1H), 6.99-6.95 (m, 1H), 4.92 (s, 1H), 4.79-4.66 (m, 2H), 4.60 (d, J=9.3 Hz, 1H), 4.52 (t, J=8.3 Hz, 1H), 4.36 (s, 1H), 4.32-4.21 (m, 2H), 4.18 (s, 2H), 3.81 (d, J=4.2 Hz, 4H), 3.68-3.64 (m, 2H), 3.36-3.34 (m, 1H), 2.66 (s, 3H), 2.47 (s, 3H), 2.11-2.04 (m, 3H), 1,98-1.92 (m, 1H), 1.90-1.85 (m, 2H), 1.45-1.39 (m, 2H), 1.37-1.31 (m, 2H), 1.31-1.24 (m, 2H), 1.23-1.20 (m, 2H), 0.96 (s, 9H).

### Example A266. 2-amino-3-cyano-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)-1-methylcyclohexyl) carbamoyl)pyridin-3-yl)-2,3-dihydrobenzofuran-3-yl)-N-methylquinoline-6-carboxamide

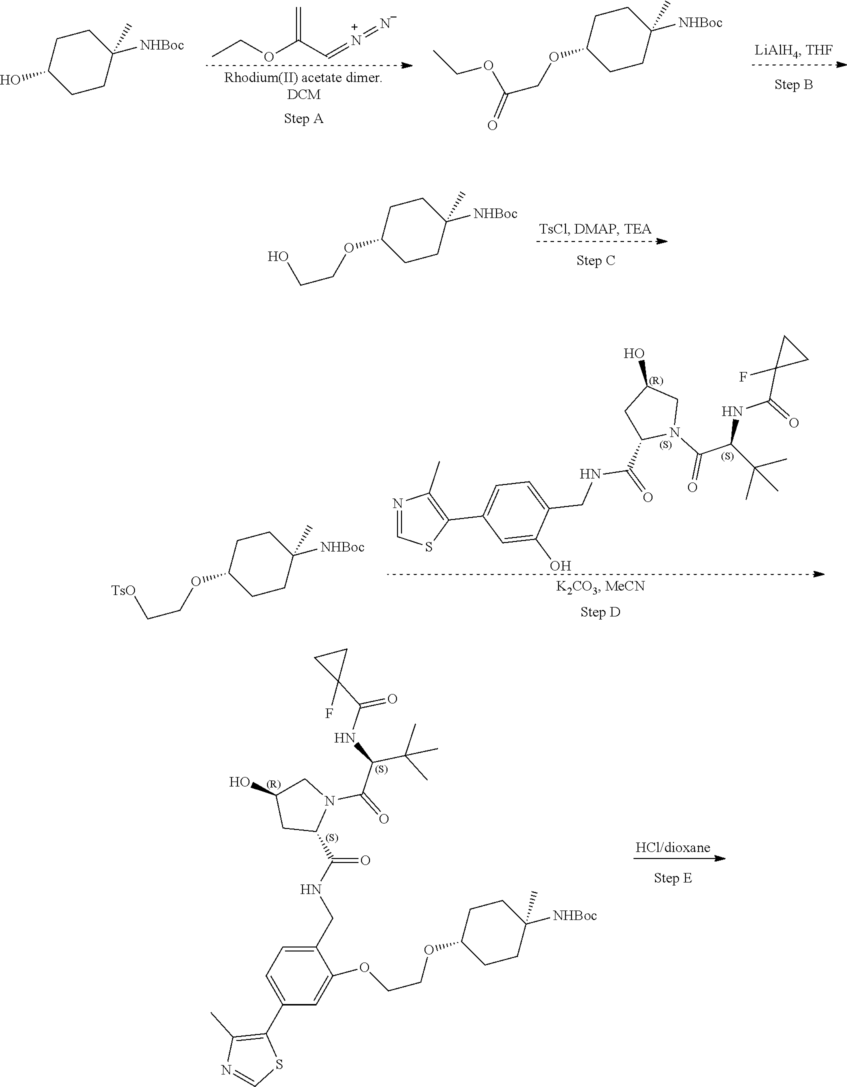

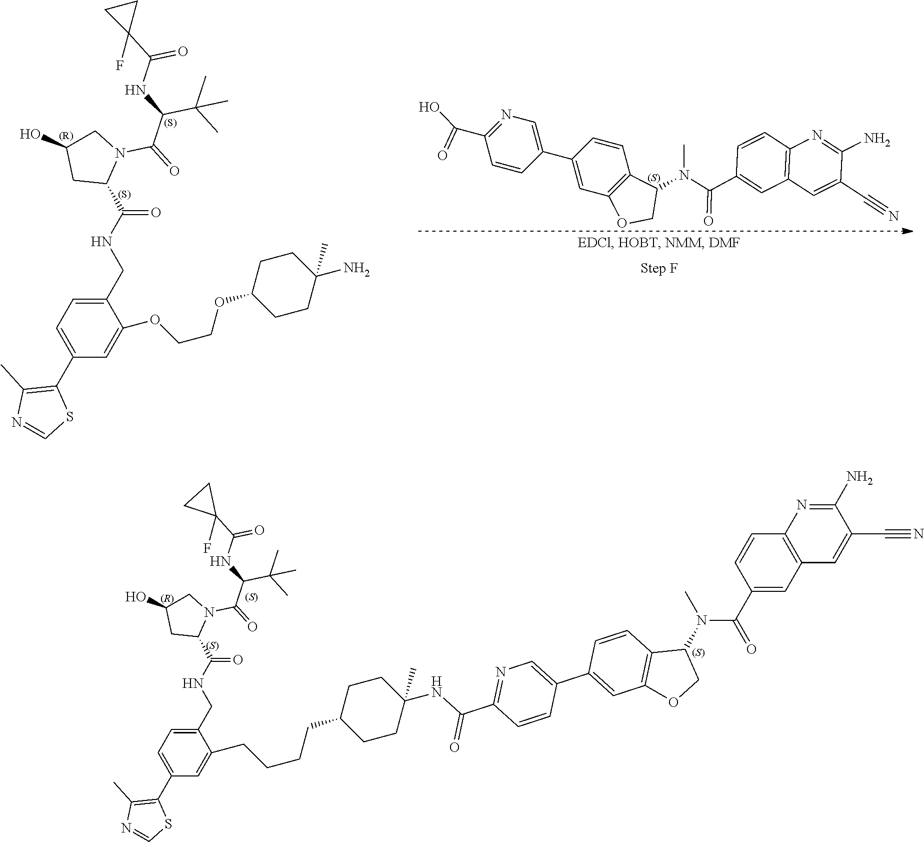

Example A266 was synthesized following the same procedures as described in Example A140 (2.2 mg, 1% yield over 6 steps) as a white solid. LC-MS (ESI): mass calcd. for C₆₁H₆₇FN₁₀O₉S, 1134.5/z found, 1136.0 [M+H]⁺. ¹H NMR (400 MHz, DMSO-d₆) δ 8.96 (s, 1H), 8.94 (s, 1H), 8.82 (s, 1H), 8.51-8.42 (m, 1H), 8.32-8.26 (m, 1H), 8.07 (d, J=8.3 Hz, 1H), 7.99 (d, J=13.1 Hz, 2H), 7.85-7.78 (m, 1H), 7.62-7.53 (m, 2H), 7.49-7.36 (m, 4H), 7.34 (s, 1H), 7.27 (d, J=6.6 Hz, 1H), 7.03 (s, 1H), 6.95 (d, J=7.7 Hz, 1H), 4.70-4.66 (m, 1H), 4.60-4.56 (m, 1H), 4.52-4.48 (m, 1H), 4.35-4.32 (m, 1H), 4.29-4.24 (m, 1H), 4.23-4.20 (m, 1H), 4.16-4.13 (m, 2H), 3.80-3.78 (m, 4H), 3.63-3.59 (m, 2H), 3.44-3.40 (m, 1H), 2.67 (s, 3H), 2.44 (s, 3H), 2.37-2.35 (m, 1H), 2.34-2.32 (m, 1H), 2.10-2.04 (m, 1H), 1.92-1.83 (m, 3H), 1.46-1.42 (m, 1H), 1.40 (s, 3H), 1.39-1.36 (m, 2H), 1.35-1.31 (m, 2H), 1.24-1.19 (m, 3H), 0.94 (s, 9H).

### Example A267. 2-amino-3-ethynyl-N-((S)-6-(6-(4-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidine-4-carbonyl)piperazine-1-carbonyl)pyridin-3-yl)-2,3-dihydrobenzofuran-3-yl)-N-methylquinoline-6-carboxamide

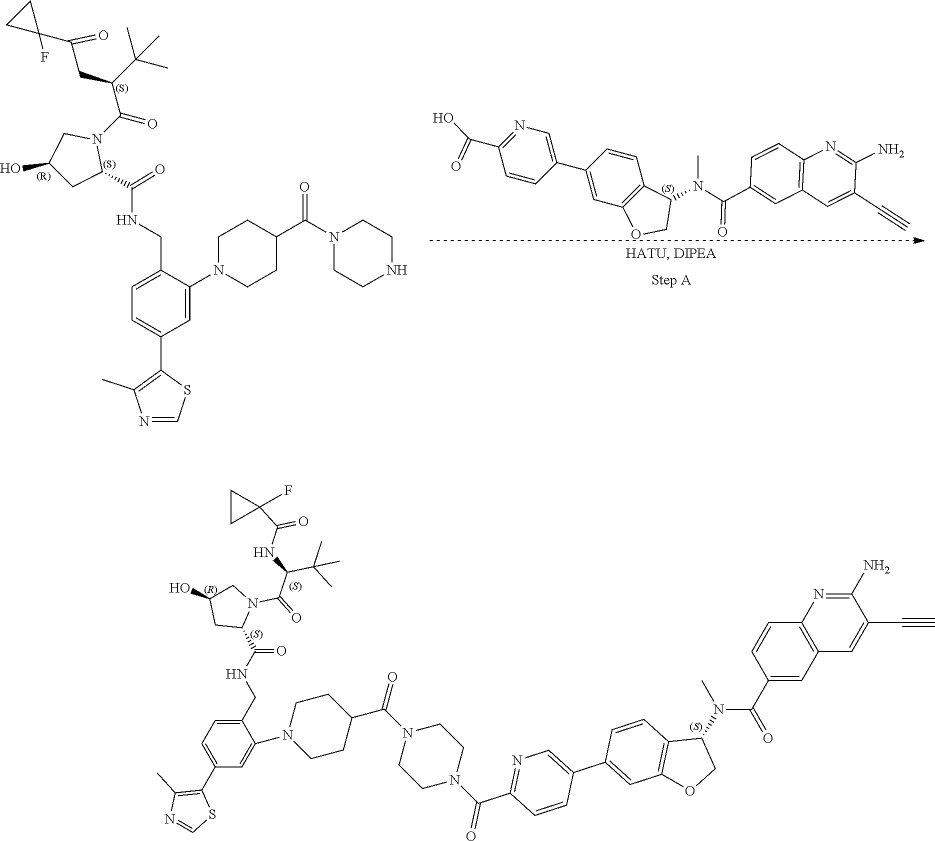

Example A267 was synthesized following the same procedures as described in Example A201 (3.8 mg, 7% yield over 1 steps) as a white solid. LC-MS (ESI): mass calcd. for C₆₃H₆₈FN₁₁O₈S, 1157.50; m/z found, 1158.5 [M+H]⁺. ¹H NMR (400 MHz, DMSO-d₆) δ 8.99 (d, J=5.6 Hz, 1H), 8.93 (s, 1H), 8.67 (s, 1H), 8.54 (s, 2H), 8.25 (d, J=9.8 Hz, 1H), 8.07 (s, 1H), 7.90 (d, J=10.0 Hz, 1H), 7.74 (s, 2H), 7.56 (d, J=7.9 Hz, 1H), 7.50-7.32 (m, 3H), 7.29 (d, J=9.1 Hz, 1H), 7.13 (s, 2H), 4.95 (s, 1H), 4.80 (s, 1H), 4.68 (d, J=9.4 Hz, 1H), 4.60 (d, J=9.3 Hz, 1H), 4.49 (t, J=8.3 Hz, 1H), 4.40-4.24 (m, 3H), 3.54-3.40 (m, 15H), 3.11 (s, 3H), 2.80 (s, 3H), 2.67 (s, 3H), 2.46 (d, J=8.5 Hz, 3H), 2.11-2.06 (m, 1H), 1.97-1.89 (m, 1H), 1.86-1.72 (m, 4H), 1.41-1.31 (m, 2H), 1.22 (dd, J=8.2, 2.9 Hz, 2H), 0.98 (s, 9H).

### Example A268. 2-amino-3-ethynyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-isopropylquinoline-6-carboxamide

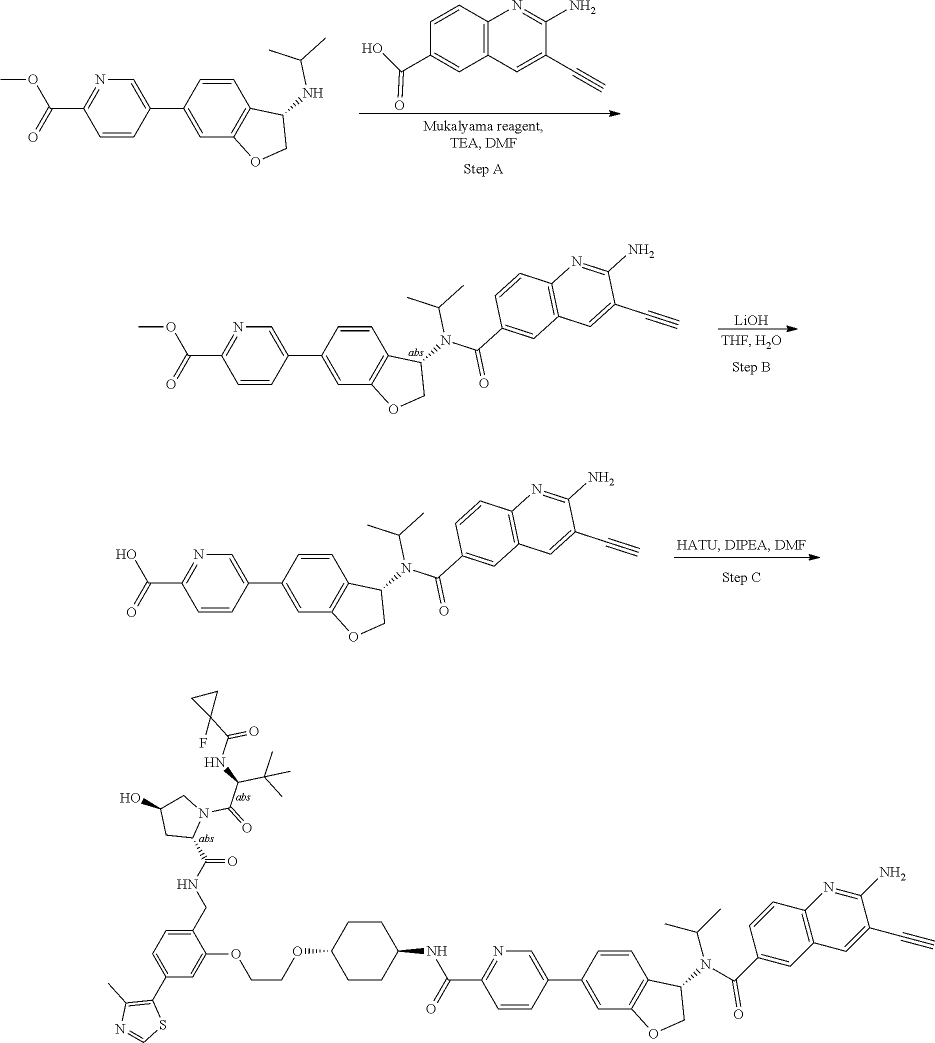

Example A268 was synthesized following the same procedures as described in Example A252 (4.5 mg, 4% yield over 3 steps) as a white solid. LC-MS (ESI): mass calcd. for C₆₃H₇₀FN₉O₉S, 1147.50; m/z found, 1148.9 [M+H]⁺. ¹HNMR (400 MHz, DMSO-d₆) δ 8.99 (s, 1H), 8.89 (s, 1H), 8.62 (s, 1H), 8.48 (dd, J=11.9, 7.2 Hz, 2H), 8.37-8.18 (m, 2H), 8.07 (d, J=8.2 Hz, 1H), 7.88 (s, 1H), 7.70 (s, 2H), 7.38 (dd, J=18.5, 6.1 Hz, 2H), 7.31-7.25 (m, 2H), 7.19 (d, J=7.4 Hz, 1H), 7.05 (d, J=1.0 Hz, 1H), 6.97 (d, J=8.5 Hz, 1H), 5.43 (s, 1H), 4.91 (s, 1H), 4.76 (s, 1H), 4.68 (s, 1H), 4.59 (d, J=9.3 Hz, 1H), 4.52 (t, J=8.2 Hz, 1H), 4.36 (s, 1H), 4.29 (d, J=6.2 Hz, 1H), 4.24 (d, J=5.7 Hz, 1H), 4.17 (dd, J=4.7, 3.8 Hz, 2H), 3.68-3.57 (m, 6H), 3.41-3.31 (m, 2H), 2.47 (s, 3H), 2.12-2.02 (m, 3H), 1.97-1.90 (m, 1H), 1.86 (d, J=11.4 Hz, 2H), 1.51 (d, J=12.1 Hz, 2H), 1.43-1.25 (m, 8H), 1.24-1.16 (m, 4H), 0.95 (s, 9H).

### Example A269. 2-amino-3-cyano-N-((S)-6-(6-(((4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl)-2,5-dihydrofuran-3-yl)methyl)carbamoyl)pyridin-3-yl)-2,3-dihydrobenzofuran-3-yl)-N-methylquinoline-6-carboxamide

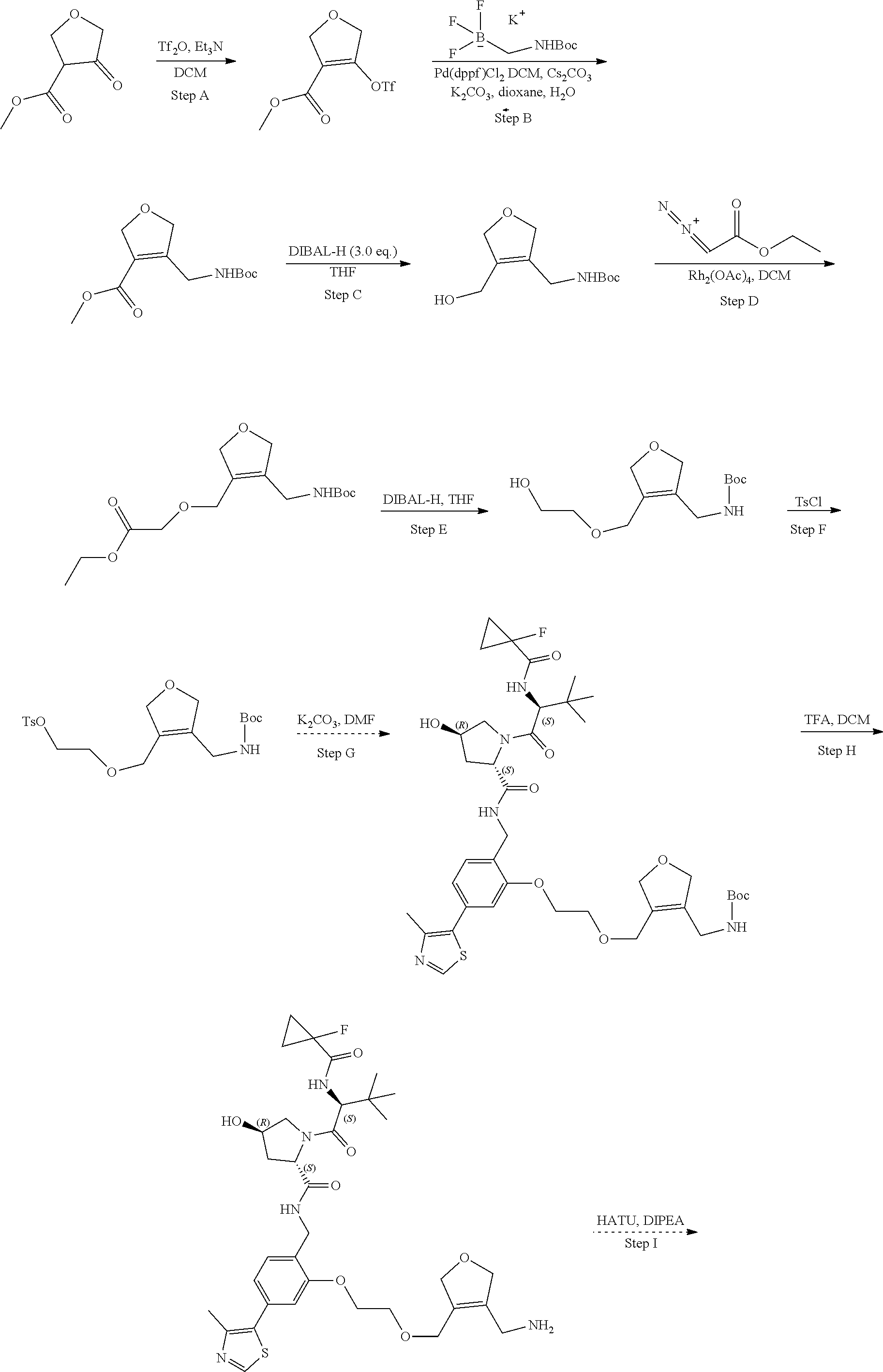

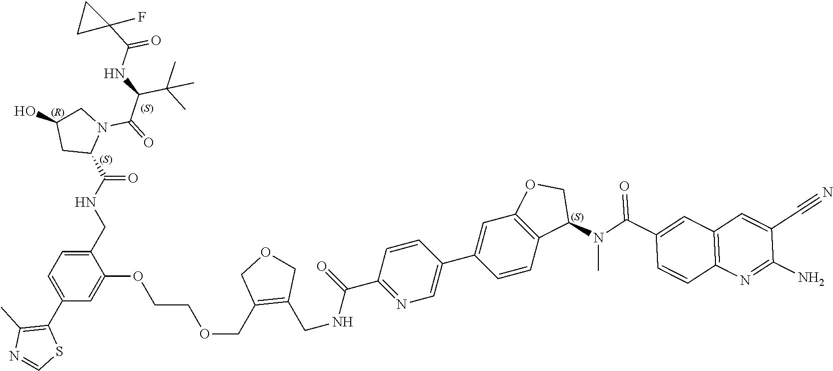

Step A: methyl 4-(((trifluoromethyl)sulfonyl)oxy)-2,5-dihydrofuran-3-carboxylate

To a solution of methyl 4-oxotetrahydrofuran-3-carboxylate (7.0 g, 48.6 mmol, 1.0 eq.) and N-ethyl-N-isopropylpropan-2-amine (7.5 g, 58.3 mmol, 1.2 eq.) in DCM (100 mL) was slowly added trifluoromethanesulfonic anhydride (16.4 g, 58.3 mmol, 1.2 eq.) at −78° C., the reaction was stirred at −78° C. for 2 hours under N₂. The reaction was quenched with water (100 mL), extracted with DCM (70 mL×3), the organic phase was dried over Na₂SO₄, filtered and concentrated in vacuo. The residue was purified by silica gel chromatography (100-200 mesh silica gel, 0-40% EtOAc in PE) to give methyl 4-(((trifluoromethyl)sulfonyl)oxy)-2,5-dihydrofuran-3-carboxylate (12.0 g, 89% yield) as a yellow oil. LC-MS (ESI): mass calcd. For: C₇H₇F₃O₆S, 276.0; m/z found, 277 [M+H]⁺.

Step B: methyl 4-(((tert-butoxycarbonyl)amino)methyl)-2,5-dihydrofuran-3-carboxylate

A solution of methyl 4-(((trifluoromethyl)sulfonyl)oxy)-2,5-dihydrofuran-3-carboxylate (2.0 g, 7.24 mmol, 1.0 eq.), Potassium (aminomethyl) trifluoroborate, N-BOC protected (2.6 g, 10.9 mmol, 1.5 eq.), PdCl₂ (dppf)-CH₂Cl₂ (296 mg, 362 μmol, 0.05 eq.) and Cs₂CO₃ (7.1 g, 21.7 mmol, 3.0 eq.) in Toluene (30.0 mL) and H₂O (1.0 mL) was stirred at 110° C. for 16 hours under N2. The reaction was concentrated in vacuo, the residue was purified by silica gel chromatography (100-200 mesh silica gel, 0-30% EtOAc in PE) to give methyl 4-(tert-butoxycarbonyl)amino)methyl)-2,5-dihydrofuran-3-carboxylate (1.2 g, 64% yield) as a yellow solid. LC-MS (ESI): mass calcd. For: C₁₂H19NO₅, 257.1; m/z found, 258 [M+H]⁺.

Step C: tert-butyl ((4-(hydroxymethyl)-2,5-dihydrofuran-3-yl)methyl)carbamate

To a solution of methyl 4-(((tert-butoxycarbonyl)amino)methyl)-2,5-dihydrofuran-3-carboxylate (1.5 g, 5.83 mmol, 1.0 eq.) in DCM (30 mL) was added DIBAL-H (11.7 mL, 17.5 mmol, 3.0 eq.) at −78° C. The reaction was stirred at −78° C. for 1 hour. The reaction was quenched by Sodiumsulfafedecahydrate (1.9 g, 5.83 mmol, 1.0 eq.), then filtered and the filtrate was concentrated in vacuo. The residue was purified by silica gel chromatography (100-200 mesh silica gel, 0-50% EtOAc in PE) to give tert-butyl ((4-(hydroxymethyl)-2,5-dihydrofuran-3-yl)methyl)carbamate (1 g, 75% yield) as a yellow oil. LC-MS (ESI): mass calcd. For: C11H19NO_{4, 229.1}; m/z found, 230 [M+H]⁺.

Step D: ethyl 2-((4-(((tert-butoxycarbonyl)amino)methyl)-2,5-dihydrofuran-3-yl)methoxy)acetate

To a solution of tert-butyl ((4-(hydroxymethyl)-2,5-dihydrofuran-3-yl)methyl)carbamate (200 mg, 872 μmol, 1.0 eq.), Rhodium (II) AcetateDimer (19.3 mg, 43.6 μmol, 0.05 eq.) in DCM (10.0 mL) was added ethyl 2-diazoacetate (299 mg, 2.62 mmol, 3.0 eq.). The reaction was stirred at room temperature for 16 hours under N₂. The reaction was concentrated in vacuo, the residue was purified by silica gel chromatography (100-200 mesh silica gel, 0-30% EtOAc in PE) to give ethyl 2-((4-(((tert-butoxycarbonyl)amino)methyl)-2,5-dihydrofuran-3-yl)methoxy)acetate (90 mg, 33% yield) as a yellow oil. LC-MS (ESI): mass calcd. For: C₁₅H₂₅NO₆, 315.2; m/z found, 316 [M+H]⁺.

Step E: tert-butyl ((4-((2-hydroxyethoxy)methyl)-2,5-dihydrofuran-3-yl)methyl)carbamate

To a solution of ethyl 2-((4-(((tert-butoxycarbonyl)amino)methyl)-2,5-dihydrofuran-3-yl)methoxy)acetate (90 mg, 285 μmol, 1.0 eq.) in DCM (8 mL) was slowly added LAH (21.7 mg, 571 μmol, 2.0 eq.) at −78° C. The reaction was stirred at −78° C. for 1 hour under N₂. The reaction was quenched by Sodiumsulfafedecahydrate (91.9 mg, 285 μmol, 1.0 eq.), then filtered and the filtrate was concentrated in vacuo to give the tert-butyl ((4-((2-hydroxyethoxy)methyl)-2,5-dihydrofuran-3-yl)methyl)carbamate (75 mg, 96% yield) as a yellow oil. LC-MS (ESI): mass calcd. For: C₁₃H23NO₅, 273.2; m/z found, 274 [M+H]⁺.

Step F: 2-((4-(((tert-butoxycarbonyl)amino)methyl)-2,5-dihydrofuran-3-yl)methoxy)ethyl 4-methylbenzenesulfonate

To a solution of tert-butyl ((4-((2-hydroxyethoxy)methyl)-2,5-dihydrofuran-3-yl)methyl)carbamate (40.0 mg, 146 μmol, 1.0 eq.), N,N-dimethylpyridin-4-amine (1.8 mg, 14.6 μmol, 0.1 eq.) and triethylamine (14.8 mg, 146 μmol, 0.1 eq.) in DCM (8 mL) was added 4-methylbenzenesulfonyl chloride (33.5 mg, 176 μmol, 1.2 eq.) at 0° C. The reaction was stirred at 0° C. for 1 hour. The reaction was quenched with water (20 mL), extracted with EtOAc (20 mL×3), the organic phase was dried over Na₂SO₄, filtered and concentrated in vacuo. The reaction was purified by silica gel chromatography (100-200 mesh silica gel, 0-40% EtOAc in PE) to give 2-((4-(((tert-butoxycarbonyl)amino)methyl)-2,5-dihydrofuran-3-yl)methoxy)ethyl 4-methylbenzenesulfonate (30 mg, 48% yield) as a yellow oil. LC-MS (ESI): mass calcd. For: C20H₂₉NO₇S, 427.2; m/z found, 428 [M+H]⁺.

Step G: tert-butyl ((4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl)-2,5-dihydrofuran-3-yl)methyl)carbamate

A solution of 2-((4-(((tert-butoxycarbonyl)amino)methyl)-2,5-dihydrofuran-3-yl)methoxy)ethyl 4-methylbenzenesulfonate (30.0 mg, 70.2 μmol, 1.0 eq.), K₂CO₃ (29.1 mg, 211 μmol, 3.0 eq.) and (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (37.4 mg, 70.2 μmol, 1.0 eq.) in DMF (5.0 mL) was stirred at 80° C. for 16 hours under N₂. The reaction was quenched with water (10 mL), extracted with EtOAc (20 mL×3), the organic phase was dried over Na₂SO₄, filtered and concentrated in vacuo. The residue was purified by silica gel chromatography (100-200 mesh silica gel, 0-80% EtOAc in PE) to give tert-butyl ((4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl)-2,5-dihydrofuran-3-yl)methyl)carbamate (30 mg, 54% yield) as a yellow solid. LC-MS (ESI): mass calcd. For: C₃₉H54FN509S, 787.4; m/z found, 788 [M+H]⁺.

Step H: (2S,4R)-N-(2-(2-((4-(aminomethyl)-2,5-dihydrofuran-3-yl)methoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of tert-butyl ((4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl)-2,5-dihydrofuran-3-yl)methyl)carbamate (30 mg, 38.1 μmol, 1.0 eq.) in DCM (2.0 mL) was added TFA (0.2 mL). The reaction was stirred at room temperature for 1 hour. The reaction was purified by prep-HPLC (C18, MeCN/H20, 0.1% HCl, 0 to 55% gradient, 30 min run) to afford the (2S,4R)-N-(2-(2-((4-(aminomethyl)-2,5-dihydrofuran-3-yl)methoxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20 mg, 76% yield) as a yellow solid. LC-MS (ESI): mass calcd. For: C₃₄H₄₆FN₅O₇S, 687.4; m/z found, 688 [M+H]⁺.

Step 1:2-amino-3-cyano-N-((S)-6-(6-(((4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl)-2,5-dihydrofuran-3-yl)methyl)carbamoyl)pyridin-3-yl)-2,3-dihydrobenzofuran-3-yl)-N-methylquinoline-6-carboxamide

Example A269 was synthesized following the same procedures as described in Example A23 (3.0 mg, 12% yield) as a white solid. LC-MS (ESI): mass calcd, for C₆₀H₆₃FN₁₀O₁₀S, 1134.4; m/z found, 1135.7 [M+H]⁺. ¹H NMR (400 MHz, DMSO-d₆) δ 9.10 (t, J=6.2 Hz, 1H), 8.97 (s, 1H), 8.91 (s, 1H), 8.81 (s, 1H), 8.50 (t, J=5.7 Hz, 1H), 8.28-8.24 (m, 1H), 8.07 (d, J=8.2 Hz, 1H), 7.97 (s, 1H), 7.84-7.79 (m, 1H), 7.59 (d, J=8.9 Hz, 1H), 7.55 (d, J=8.1 Hz, 1H), 7.42-7.37 (m, 3H), 7.34 (s, 1H), 7.29 (dd, J=9.5, 2.5 Hz, 1H), 7.04 (s, 1H), 6.96 (d, J=7.8 Hz, 1H), 4.87-4.66 (m, 2H), 4.61-4.49 (m, 7H), 4.32 (s, 4H), 4.22 (dd, J=9.9, 5.6 Hz, 3H), 4.10 (d, J=6.5 Hz, 2H), 3.78 (s, 4H), 2.67 (s, 3H), 2.44 (s, 3H), 2.11-2.05 (m, 1H), 1.95-1.88 (m, 1H), 1.38 (d, J=10.0 Hz, 1H), 1.34 (d, J=9.7 Hz, 1H), 1.21 (d, J=8.4 Hz, 2H), 0.95 (s, 9H).

### Example A270. 2-amino-3-ethynyl-N-((3S)-6-(6-(3-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-3,8-diazabicyclo[3.2.1]octane-8-carbonyl)pyridin-3-yl)-2,3-dihydrobenzofuran-3-yl)-N-methylquinoline-6-carboxamide

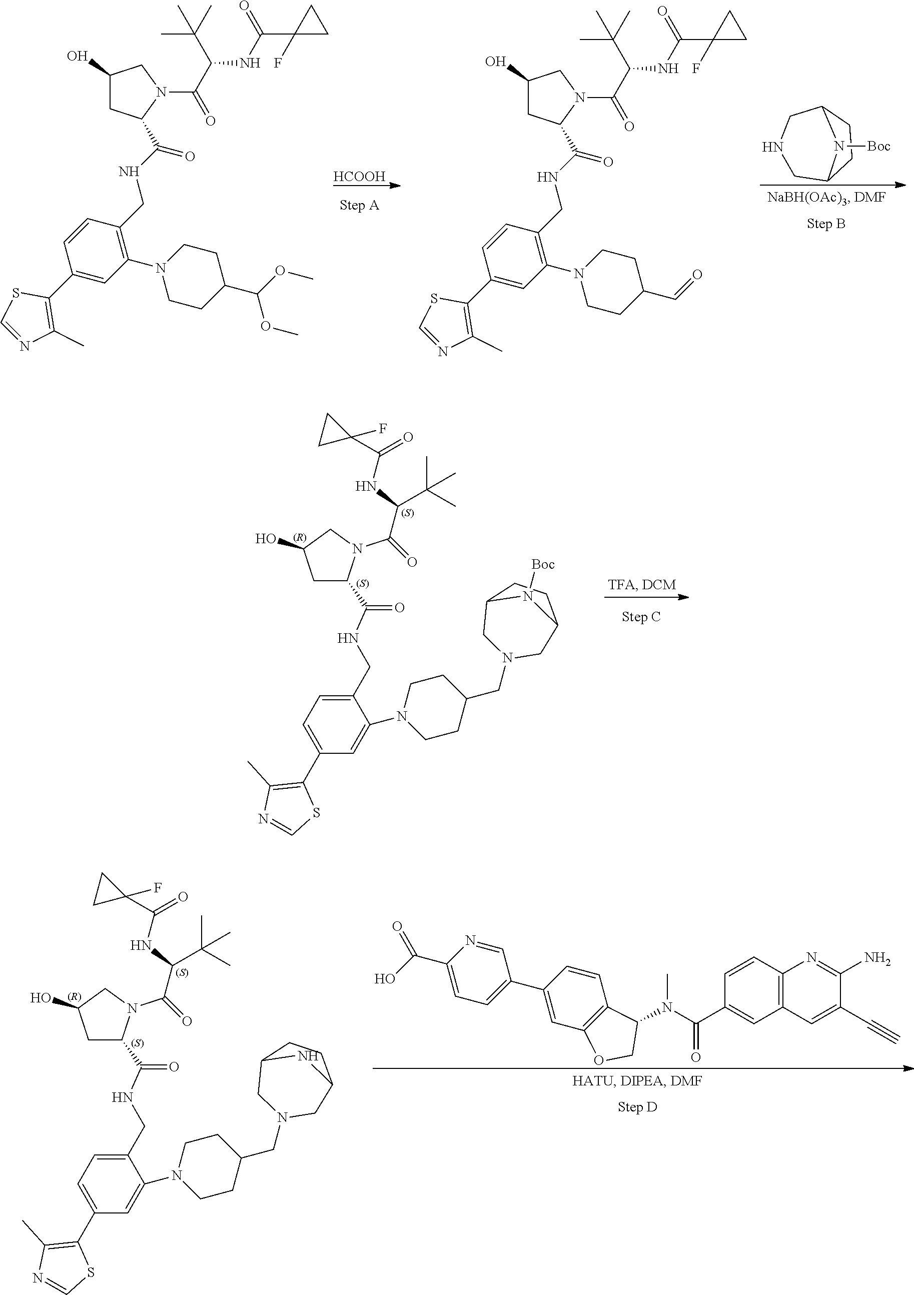

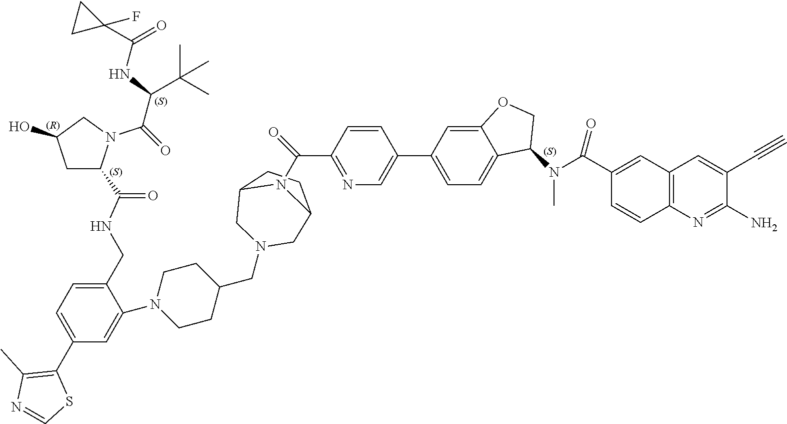

Example A270 was synthesized following the same procedures as described in Example A107 (5.0 mg, 4% yield over 4 steps) as a yellow solid. LC-MS (ESI): mass calcd. for C₆₅H₇₂FN₁₁O₇S, 1169.53; m/z found, 1170.9 [M+H]⁺. ¹HNMR (400 MHz, DMSO-d₆) δ 9.31 (s, 1H), 8.99 (s, 1H), 8.96 (s, 1H), 8.62 (s, 1H), 8.54-8.48 (m, 1H), 8.36-8.20 (m, 2H), 8.04 (s, 1H), 7.96 (d, J=8.0 Hz, 1H), 7.87 (d, J=8.6 Hz, 1H), 7.75-7.67 (m, 1H), 7.56 (d, J=7.9 Hz, 1H), 7.46-7.39 (m, 2H), 7.36 (s, 1H), 7.26 (dd, J=9.2, 2.2 Hz, 1H), 7.13 (d, J=6.3 Hz, 2H), 5.21 (s, 1H), 4.91 (s, 2H), 4.75-4.65 (m, 2H), 4.60 (d, J=9.0 Hz, 1H), 4.49 (t, J=7.0 Hz, 1H), 4.39-4.32 (m, 3H), 3.67-3.60 (m, 4H), 3.50-3.28 (m, 4H), 3.19-3.04 (m, 4H), 2.74-2.63 (m, 5H), 2.46 (s, 3H), 2.15-2.01 (m, 5H), 1.95-1.83 (m, 4H), 1.48-1.32 (m, 4H), 1.21 (dd, J=8.1, 2.4 Hz, 2H), 0.97 (s, 9H).

### Example A271. 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-3-(1,1,1-trifluoropropan-2-yl)quinoline-6-carboxamide

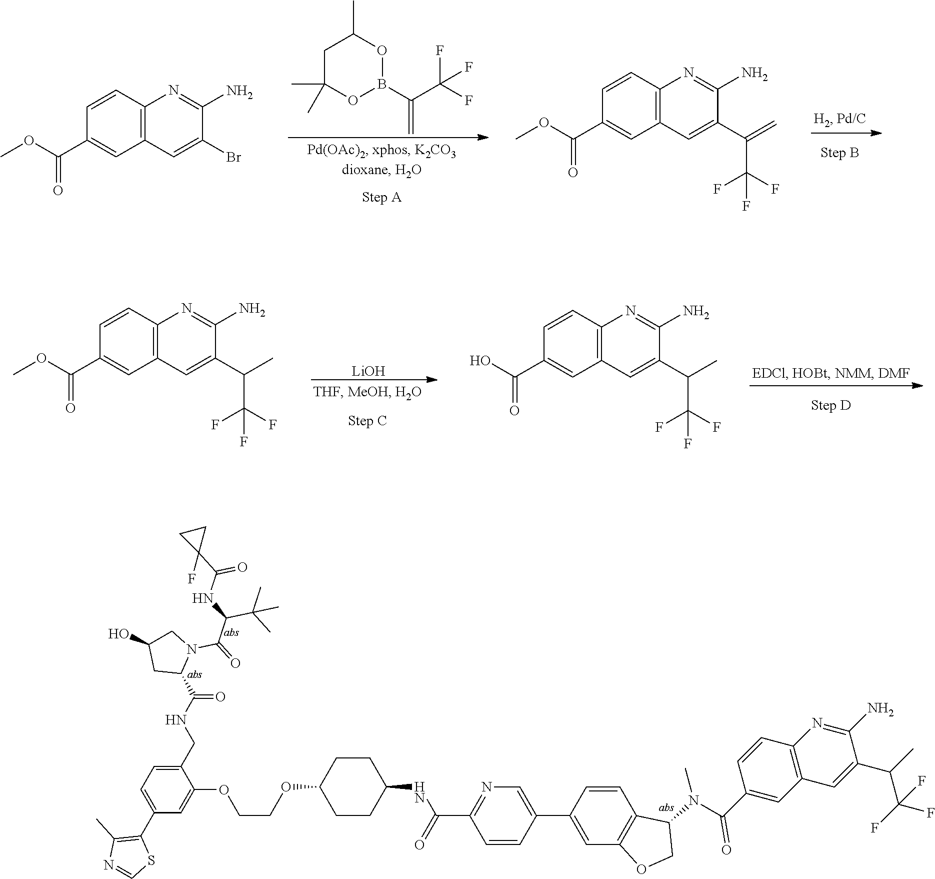

Step A: methyl 2-amino-3-(3,3,3-trifluoroprop-1-en-2-yl)quinoline-6-carboxylate

To a mixture of methyl 2-amino-3-bromoquinoline-6-carboxylate (50 mg, 178 μmol, 1.0 eq.) and 4,4,6-trimethyl-2-(3,3,3-trifluoroprop-1-en-2-yl)-1,3,2-dioxaborinane (40 mg, 178 μmol, 1.0 eq.) in 1,4-Dioxane (2 mL) and Water (0.1 mL) was added 2-(Dicyclohexylphosphanyl)-2′,4′,6′-tris (isopropyl) biphenyl (9 mg, 17.8 μmol, 0.1 eq.), diacetyl (oxo) palladium (8 mg, 35.6 μmol, 0.2 eq.) and K₂CO₃ (50 mg, 356 μmol, 2.0 eq.). The mixture stirred at 110° C. for 4 hours under N₂. The mixture was filtered and concentrated under reduced pressure. The residue was purified by flash column chromatography on silica gel (silica gel, 0-2% MeOH in DCM) to give methyl 2-amino-3-(3,3,3-trifluoroprop-1-en-2-yl)quinoline-6-carboxylate (50 mg, 94% yield) as a yellow solid. LC-MS (ESI): mass calcd. For C₁₄H₁₁F₃N₂O₂, 296.08; m/z found, 297.1 [M+H]⁺

Step B: methyl 2-amino-3-(1, 1, 1-trifluoropropan-2-yl)quinoline-6-carboxylate

To a solution of methyl 2-amino-3-(3,3,3-trifluoroprop-1-en-2-yl)quinoline-6-carboxylate (50 mg, 169 μmol, 1.0 eq.) in MeOH (8 mL) was added Pd/C (10 mg, 94.0 μmol, 0.5 eq.). The mixture was then stirred at room temperature for 16 hours under H₂. The reaction was filtered and concentrated to give methyl 2-amino-3-(1,1,1-trifluoropropan-2-yl)quinoline-6-carboxylate (50 mg, 99% yield) as a yellow solid. LC-MS (ESI): mass calcd. For C₁₄H₁₃F₃N₂O₂, 298.09; m/z found, 299.1 [M+H]⁺

Step C: 2-amino-3-(1,1,1-trifluoropropan-2-yl)quinoline-6-carboxylic acid

To a mixture of methyl 2-amino-3-(1,1,1-trifluoropropan-2-yl)quinoline-6-carboxylate (50 mg, 168 μmol, 1.0 eq.) in THF (2 mL), MeOH (1 mL) and Water (1 mL) was added Lithium hydroxide monohydrate (14 mg, 9.32 μL, 335 μmol, 2.0 eq.) at 0° C. The mixture was stirred at room temperature for 2 hours. The mixture was concentrated to afford 2-amino-3-(1,1,1-trifluoropropan-2-yl)quinoline-6-carboxylic acid (40 mg, 83% yield) as a yellow solid. LC-MS (ESI): mass calcd. For C₁₃H₁₁F₃N₂O₂, 284.08; m/z found, 285.0 [M+H]⁺

Step D: 2-amino-N-(S)-6-(6-(((15,4S)-4-(2-(2-(2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methyl-3-(1,1,1-trifluoropropan-2-yl)quinoline-6-carboxamide

Example A271 was synthesized following the same procedures as described in Example A29 (1.0 mg, 1% yield) as a yellow solid. LC-MS (ESI): mass calcd, for C₆₂H₆₉F₄N₉O₉S, 1191.49; m/z found, 1192.9 [M+H]⁺, ¹HNMR (400 MHz, DMSO-d₆) δ 8.99 (s, 1H), 8.93 (s, 1H), 8.68 (s, 1H), 8.52-8.47 (m, 2H), 8.28 (d, J=8.0 Hz, 1H), 8.17 (s, 1H), 8.09 (d, J=6.9 Hz, 1H), 7.91 (s, 1H), 7.76 (s, 1H), 7.57 (d, J=7.9 Hz, 1H), 7.41 (d, J=7.1 Hz, 2H), 7.36 (s, 1H), 7.30 (d, J=8.3 Hz, 1H), 7.06 (s, 1H), 6.97 (d, J=6.9 Hz, 1H), 4.70 (s, 1H), 4.60 (d, J=12.0 Hz, 1H), 4.52 (t, J=7.8 Hz, 1H), 4.36 (s, 1H), 4.30 (d, J=7.2 Hz, 1H), 4.25 (d, J=5.0 Hz, 1H), 4.21-4.12 (m, 3H), 3.84-3.78 (m, 4H), 3.66-3.61 (m, 4H), 2.69-2.66 (m, 3H), 2.47 (s, 3H), 2.10-2.02 (m, 4H), 1.88-1.83 (m, 2H), 1.57 (d, J=5.6 Hz, 2H), 1.54-1.48 (m, 2H), 1.38-1.30 (m, 4H), 1.24 (s, 2H), 0.96 (s, 9H).

### Example A272. N-((S)-6-(6-(((15,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methyl-4-(methylamino)-1,3-dihydrofuro[3,4-c][1,7]naphthyridine-8-carboxamide

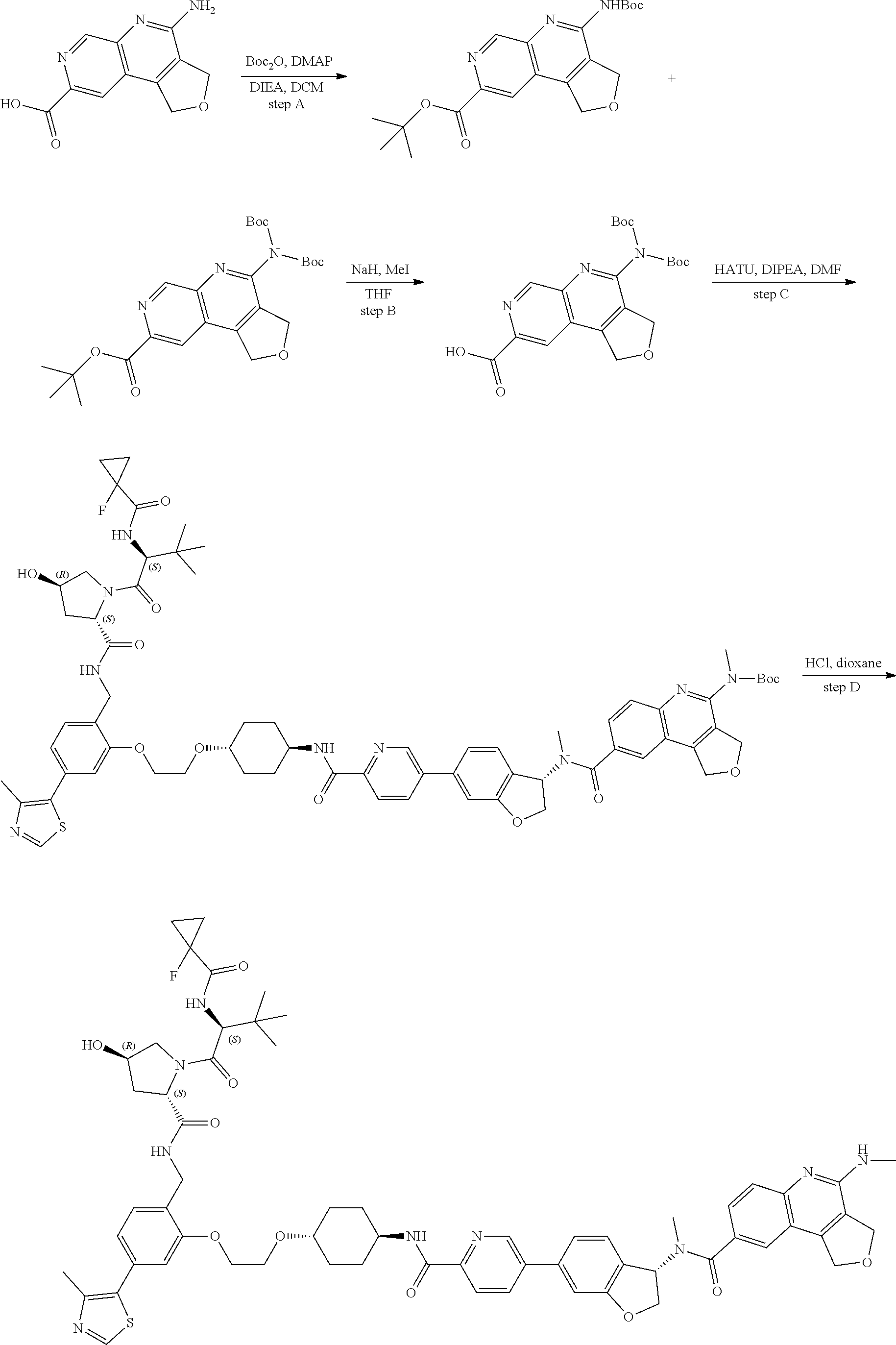

Example A272 was synthesized following the same procedures as described in Example

A249 (2.2 mg, 1% yield over 4 steps) as a white solid. LC-MS (ESI): mass calcd. for C₆₁H₆₉FN₁₀O₁₀S, 1152.5/z found, 1154.8 [M+H]⁺, ¹H NMR (400 MHz, MeOD-d) δ 9.25-9.13 (m, 1H), 8.90 (s, 1H), 8.87 (s, 1H), 8.21-8.17 (m, 1H), 8.16-8.12 (m, 1H), 8.04-7.92 (m, 1H), 7.63-7.56 (m, 1H), 7.55-7.49 (m, 1H), 7.49-7.46 (m, 1H), 7.36-7.31 (m, 1H), 7.27-7.21 (m, 1H), 7.07 (s, 1H), 7.05-7.01 (m, 1H), 5.50 (s, 2H), 5.17 (s, 2H), 4.76-4.73 (m, 2H), 4.66-4.61 (m, 1H), 4.53-4.47 (m, 2H), 4.46-4.40 (m, 1H), 4.25-4.21 (m, 2H), 3.97-3.90 (m, 3H), 3.87-3.78 (m, 2H), 3.58-3.44 (m, 1H), 3.37-3.32 (m, 2H), 3.25-3.20 (m, 3H), 2.87-2.79 (m, 3H), 2.50 (s, 3H), 2.30-2.22 (m, 1H), 2.20-2.11 (m, 3H), 2.07-2.00 (m, 2H), 1.54-1.45 (m, 4H), 1.34-1.28 (m, 4H), 1.03 (s, 9H).

### Example A274. 2-amino-3-cyano-N-((S)-6-(6-((6-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)pyridin-3-yl)carbamoyl) pyridin-3-yl)-2,3-dihydrobenzofuran-3-yl)-N-methylquinoline-6-carboxamide

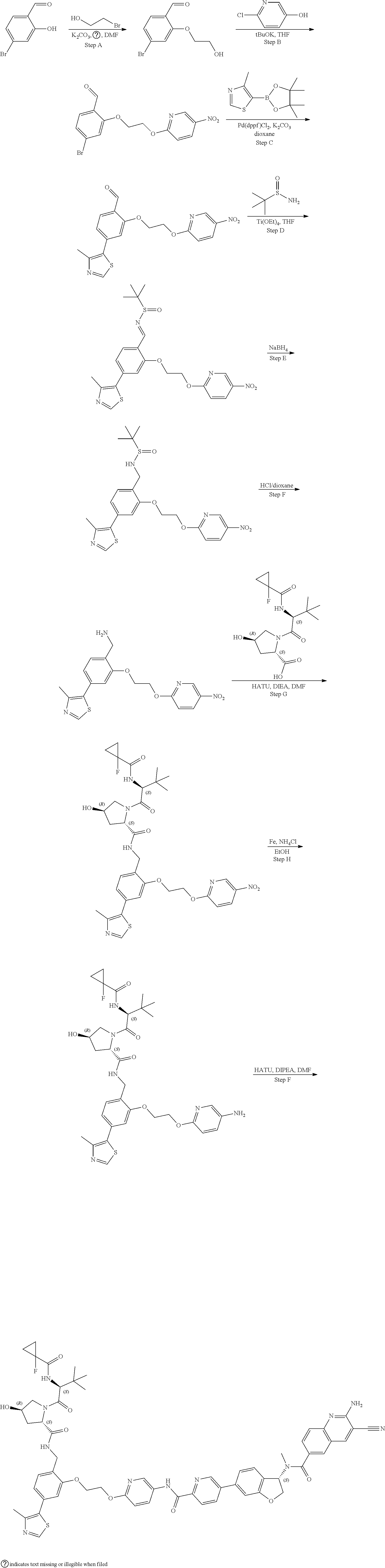

Step A: 4-bromo-2-(2-hydroxyethoxy)benzaldehyde

To a mixture of 4-bromo-2-hydroxybenzaldehyde (2.0 g, 10.0 mmol, 1.0 eq.) and 2-bromoethan-1-ol (2.5 g, 19.9 mmol, 2.0 eq.) in DMF (20 mL) were added K₂CO₃ (4.1 g, 29.8 mmol, 3.0 eq.) and KI (165 mg, 995 μmol, 0.1 eq.). The mixture was stirred at 80° C. for 24 hours. The mixture was poured into water (50 mL) and extracted with EtOAc (40 mL×3). The combined organic layers were dried over anhydrous Na₂SO₄, filtered and concentrated to dryness. The residue was purified by flash chromatography with 0-30% EtOAc in PE to afford 4-bromo-2-(2-hydroxyethoxy)benzaldehyde (2 g, 82% yield) as a white solid. LC-MS (ESI): mass calcd. For: C₉H₉BrO₃, 244.0; m/z found, 245 [M+H]⁺

Step B: 4-bromo-2-(2-((5-nitropyridin-2-yl)oxy)ethoxy)benzaldehyde

A solution of 4-bromo-2-(2-hydroxyethoxy)benzaldehyde (1.5 g, 6.12 mmol, 1.0 eq.), 2-chloro-5-nitropyridine (1.5 g, 9.18 mmol, 1.5 eq.) and potassium 2-methylpropan-2-olate (1.0 g, 9.18 mmol, 1.5 eq.) in THF (30 mL) was stirred at room temperature for 16 hours. The reaction was quenched with water (20 mL), extracted with EtOAc (30 mL×3), the organic phase was dried over Na₂SO₄, filtered and concentrated in vacuo. The residue was purified by silica gel chromatography (100-200 mesh silica gel, 0-30% EtOAc in PE) to give 4-bromo-2-(2-((5-nitropyridin-2-yl)oxy)ethoxy)benzaldehyde (0.6 g, 27% yield) as a yellow solid. LC-MS (ESI): mass calcd. For: C₁₄H₁₁BrN₂O₅, 366.0; m/z found, 367 [M+H]⁺.

Step C: 4-(4-methylthiazol-5-yl)-2-(2-((5-nitropyridin-2-yl)oxy)ethoxy)benzaldehyde

A solution of 4-bromo-2-(2-((5-nitropyridin-2-yl)oxy)ethoxy)benzaldehyde (400 mg, 1.09 mmol, 1.0 eq.), 4-methyl-5-(4,4,5,5-tetramethyl-1,3,2-dioxaborolan-2-yl) thiazole (368 mg, 1.63 mmol, 1.5 eq.), K₂CO₃ (452 mg, 3.27 mmol, 3.0 eq.) and PdCl₂ (dppf)-CH₂Cl₂ (178 mg, 218 μmol, 0.2 eq.) in 1,4-Dioxane (10.0 mL) and H₂O (1.0 mL) was stirred at 100° C. for 16 hours under N₂. The reaction was concentrated in vacuo, the residue was purified by silica gel chromatography (100-200 mesh silica gel, 0-30% EtOAc in PE) to give 4-(4-methylthiazol-5-yl)-2-(2-((5-nitropyridin-2-yl)oxy)ethoxy)benzaldehyde (0.20 g, 48% yield) as a yellow solid. LC-MS (ESI): mass calcd. For: C₁₈H₁₅N₃O₅S, 385.1; m/z found, 386 [M+H]⁺.

Step D: (E)-2-methyl-N-(4-(4-methylthiazol-5-yl)-2-(2-((5-nitropyridin-2-yl)oxy)ethoxy)benzylidene)propane-2-sulfinamide

A solution of 4-(4-methylthiazol-5-yl)-2-(2-((5-nitropyridin-2-yl)oxy)ethoxy)benzaldehyde (200 mg, 519 μmol, 1.0 eq.), 2-methylpropane-2-sulfinamide (94 mg, 778 μmol, 1.5 eq.) and Titaniumethoxide (355 mg, 1.56 mmol, 3.0 eq.) in THF (10 mL) was stirred at 70° C. for 16 hours. The reaction was diluted with water (10 mL), extracted with EtOAc (20 mL×3), the organic phase was dried over Na₂SO₄, filtered and concentrated in vacuo. The residue was purified by silica gel chromatography (100-200 mesh silica gel, 0-50% EtOAc in PE) to give (E)-2-methyl-N-(4-(4-methylthiazol-5-yl)-2-(2-((5-nitropyridin-2-yl)oxy)ethoxy)benzylidene) propane-2-sulfinamide (150 mg, 59% yield) as a yellow solid. LC-MS (ESI): mass calcd. For: C₂₂H₂₄N405S2, 488.1; m/z found, 489 [M+H]⁺.

Step E: (E)-2-methyl-N-(4-(4-methylthiazol-5-yl)-2-(2-((5-nitropyridin-2-yl)oxy)ethoxy)benzylidene)propane-2-sulfinamide

To a solution of (E)-2-methyl-N-(4-(4-methylthiazol-5-yl)-2-(2-((5-nitropyridin-2-yl)oxy)ethoxy)benzylidene)propane-2-sulfinamide (150 mg, 307 μmol, 1.0 eq.) in MeOH (5 mL) was added NaBH₄ (46.5 mg, 1.23 mmol, 4.0 eq.), the reaction was stirred at 0° C. for 2 hours. The reaction was quenched with water (20 mL), extracted with EtOAc (20 mL×3), the organic phase was dried over Na₂SO₄, filtered and concentrated in vacuo to give the 2-methyl-N-(4-(4-methylthiazol-5-yl)-2-(2-((5-nitropyridin-2-yl)oxy)ethoxy)benzyl)propane-2-sulfinamide (60 mg, 39% yield) as a yellow solid. LC-MS (ESI): mass calcd. For: C₂₂H₂₆N405S2, 490.1; m/z found, 491 [M+H]⁺.

Step F: (4-(4-methylthiazol-5-yl)-2-(2-((5-nitropyridin-2-yl)oxy)ethoxy)phenyl)methanamine

To a solution of 2-methyl-N-(4-(4-methylthiazol-5-yl)-2-(2-((5-nitropyridin-2-yl)oxy)ethoxy)benzyl)propane-2-sulfinamide (60 mg, 122 μmol, 1.0 eq.) in DCM (2.0 mL) was added TFA (0.3 mL). The mixture was stirred at room temperature for 1 hour. The reaction was concentrated in vacuo to give (4-(4-methylthiazol-5-yl)-2-(2-((5-nitropyridin-2-yl)oxy)ethoxy)phenyl)methanamine (45 mg, 95% yield) as a yellow oil. LC-MS (ESI): mass calcd. For: C₁₈H18N404S, 386.1; m/z found, 387 [M+H]⁺.

Step G: (2S, 4R)-1-((S)-2-(1-fluorocyclopropane-1-carboxamido)-3,3-dimethylbutanoyl)-4-hydroxy-N-(4-(4-methylthiazol-5-yl)-2-(2-((5-nitropyridin-2-yl)oxy)ethoxy)benzyl)pyrrolidine-2-carboxamide

A solution of (4-(4-methylthiazol-5-yl)-2-(2-((5-nitropyridin-2-yl)oxy)ethoxy)phenyl)methanamine (40 mg, 104 μmol, 1.0 eq.), (2S,4R)-1-((S)-2-(1-fluorocyclopropane-1-carboxamido)-3,3-dimethylbutanoyl)-4-hydroxypyrrolidine-2-carboxylic acid (44.5 mg, 135 μmol, 1.3 eq.), HATU (59.0 mg, 155 μmol, 1.5 eq.) and N-ethyl-N-isopropylpropan-2-amine (40.1 mg, 311 μmol, 3.0 eq.) in DMF (3.0 mL) was stirred at room temperature for 2 hours. The reaction was quenched with water (20 mL), extracted with EtOAc (20 mL×3), the organic phase was dried over Na₂SO₄, filtered and concentrated in vacuo. The residue was purified by silica gel chromatography (100-200 mesh silica gel, 0-100% EtOAc in PE) to give (2S,4R)-1-((S)-2-(1-fluorocyclopropane-1-carboxamido)-3,3-dimethylbutanoyl)-4-hydroxy-N-(4-(4-methylthiazol-5-yl)-2-(2-((5-nitropyridin-2-yl)oxy)ethoxy)benzyl)pyrrolidine-2-carboxamide (32 mg, 44% yield) as a yellow solid. LC-MS (ESI): mass calcd. For: C₃₃H₃₉FN₆O₈S, 698.3; m/z found, 699 [M+H]⁺.

Step H: (2S,4R)-N-(2-(2-((5-aminopyridin-2-yl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

A solution of (2S,4R)-1-((S)-2-(1-fluorocyclopropane-1-carboxamido)-3,3-dimethylbutanoyl)-4-hydroxy-N-(4-(4-methylthiazol-5-yl)-2-(2-((5-nitropyridin-2-yl)oxy)ethoxy)benzyl)pyrrolidine-2-carboxamide (32 mg, 45.8 μmol, 1.0 eq.), Ammonium chloride (24.5 mg, 458 μmol, 10 eq.) and iron (20.5 mg, 366 μmol, 8.0 eq.) in EtOH (2 mL) and H₂O (1 mL) was stirred at 90° C. for 1 hour. The reaction was filtered and the filtrate was diluted with water (10 mL), extracted with EtOAc (20 mL×3), the organic phase was dried over Na₂SO₄, filtered and concentrated in vacuo to give the (2S,4R)-N-(2-(2-((5-aminopyridin-2-yl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (20 mg, 65% yield) as a yellow oil. LC-MS (ESI): mass calcd. For: C33H41FN606S, 668.3; m/z found, 669 [M+H]⁺.

Step I: 2-amino-3-cyano-N-(S)-6-(6-((6-(2-(2-(28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)pyridin-3-yl)carbamoyl)pyridin-3-yl)~2,3-dihydrobenzofuran-3-yl)-N-methylquinoline-6-carboxamide

Example A274 was synthesized following the same procedures as described in Example A23 (3.5 mg, 10% yield) as a white solid. LC-MS (ESI): mass calcd, for C₅₉H58FN₁₁O9S, 1115.4; m/z found, 1116.7 [M+H]⁺. ¹H NMR (400 MHz, DMSO-d₆) δ 10.83 (s, 1H), 9.03 (s, 1H), 8.99 (s, 1H), 8.82 (s, 1H), 8.71 (d, J=2.5 Hz, 1H), 8.49 (t, J=5.9 Hz, 1H), 8.38-8.34 (m, 1H), 8.23 (dd, J=13.3, 5.3 Hz, 2H), 7.97 (s, 1H), 7.84-7.80 (m, 1H), 7.58 (d, J=7.7 Hz, 2H), 7.45-7.40 (m, 5H), 7.29 (d, J=7.1 Hz, 1H), 7.10 (s, 1H), 6.98 (d, J=7.9 Hz, 1H), 6.91 (d, J=8.9 Hz, 1H), 4.73-4.68 (m, 1H), 4.66-4.62 (m, 2H), 4.59 (d, J=9.2 Hz, 1H), 4.51 (t, J=8.3 Hz, 1H), 4.45-4.41 (m, 2H), 4.36-4.31 (m, 1H), 4.27 (d, J=6.3 Hz, 1H), 4.20 (d, J=5.7 Hz, 1H), 3.67-3.57 (m, 4H), 2.69 (s, 3H), 2.47 (s, 3H), 2.10-2.05 (m, 1H), 1.94-1.89 (m, 1H), 1.41-1.36 (m, 1H), 1.36-1.32 (m, 1H), 1.27-1.19 (m, 2H), 0.95 (s, 9H).

### Example A275. 2-amino-3-cyano-N-((S)-6-(6-(((1S,4S)-4-(4-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamoyl)pyridin-3-yl)-2,3-dihydrobenzofuran-3-yl)-N-methylquinoline-6-carboxamide

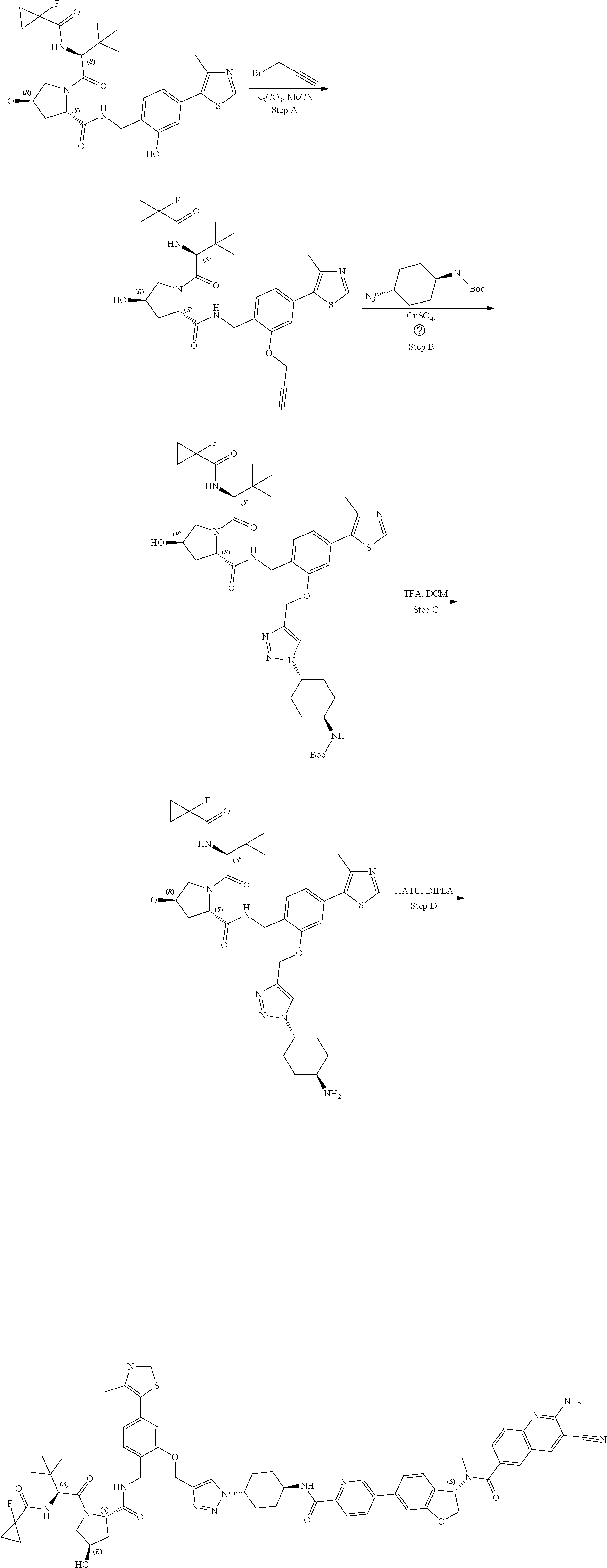

Step A: (2S,4R)-1-((S)-2-(1-fluorocyclopropane-1-carboxamido)-3,3-dimethylbutanoyl)-4-hydroxy-N-(4-(4-methylthiazol-5-yl)-2-(prop-2-yn-1-yloxy)benzyl)pyrrolidine-2-carboxamide

To a solution of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (250 mg, 469 μmol, 1.0 eq.) in MeCN (5 mL) was added3-bromoprop-1-yne (112 mg, 939 μmol, 2.0 eq.) and K₂CO₃ (130 mg, 939 μmol, 2.0 eq.). The reaction was stirred at 80° C. for 16 hours under N₂. The mixture was poured into water (50 mL), extracted with EtOAc (30 mL×3) and the layers were separated. The organic layer was washed with brine (50 mL), dried over Na₂SO₄, filtered and concentrated. The product was purified by column chromatography (silica gel, 0% MeOH in DCM~10% MeOH in DCM) to afford (2S,4R)-1-((S)-2-(1-fluorocyclopropane-1-carboxamido)-3,3-dimethylbutanoyl)-4-hydroxy-N-(4-(4-methylthiazol-5-yl)-2-(prop-2-yn-1-yloxy)benzyl)pyrrolidine-2-carboxamide (220 mg, 82% yield) as a white solid. LC-MS (ESI): mass calcd. For C29H33FN405S, 570.23; m/z found, 571.2 [M+H]⁺.

Step B: tert-butyl ((1S, 4r)-4-(4-((2-(((2S,4R)-1-((S)-2-(1-fluorocyclopropane-1-carboxamido)-3, 3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamate

To a solution of (2S,4R)-1-((S)-2-(1-fluorocyclopropane-1-carboxamido)-3,3-dimethylbutanoyl)-4-hydroxy-N-(4-(4-methylthiazol-5-yl)-2-(prop-2-yn-1-yloxy)benzyl)pyrrolidine-2-carboxamide (50 mg, 88 μmol, 1.0 eq.) in THE (1 mL): t-BuOH (1 mL): Water (1 mL) was added tert-butyl ((1r,4r)-4-azidocyclohexyl)carbamate (42 mg, 0.18 mmol, 2.0 eq.), Copper (II) sulfate anhydrous (2.8 mg, 18 μmol, 0.2 eq.) and Sodium 2-(1,2-dihydroxy-ethyl)-4-hydroxy-5-oxo-2,5-dihydro-furan-3-olate (35 mg, 0.18 mmol, 2.0 eq.). The reaction was stirred at room temperature for 2 hours. The mixture was poured into water (30 mL), extracted with EtOAc (30 mL×3) and the layers were separated. The organic layer was washed with brine (30 mL), dried over Na₂SO₄, filtered and concentrated. The product was purified by column chromatography (silica gel, 0% MeOH in DCM~10% MeOH in DCM) to afford tert-butyl ((1S,4r)-4-(4-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamate (61 mg, 86% yield) as a white solid. LC-MS (ESI): mass calcd. For C40H55FN₈O₉S, 810.39; m/z found, 811.7 [M+H]⁺.

Steps C-D: 2-amino-3-cyano-N-((S)-6-(6-(((1S,4S)-4-(4-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamoyl)pyridin-3-yl)-2,3-dihydrobenzofuran-3-yl)-N-methylquinoline-6-carboxamide

Example A275 was synthesized following the same procedures as described in Example A23 (6.8 mg, 8% yield over 2 steps) as a white solid. LC-MS (ESI): mass calcd. for C₆₁H₆₄FN13O₈S, 1157.47; m/z found, 1158.7 [M+H]⁺. ¹H NMR (400 MHz, DMSO-d₆) δ 9.01 (s, 1H), 8.95 (s, 1H), 8.83 (s, 1H), 8.66 (d, J=8.5 Hz, 1H), 8.52 (t, J=6.0 Hz, 1H), 8.37 (s, 1H), 8.29 (dd, J=8.2, 2.2 Hz, 1H), 8.11 (d, J=8.2 Hz, 1H), 7.98 (s, 1H), 7.82 (dd, J=8.7, 1.9 Hz, 1H), 7.62-7.55 (m, 2H), 7.43 (d, J=7.9 Hz, 2H), 7.36 (s, 1H), 7.31-7.23 (m, 2H), 7.02-6.96 (m, 1H), 5.27 (s, 2H), 4.71-4.68 (m, 1H), 4.61-4.58 (m, 1H), 4.54-4.49 (m, 2H), 4.36-4.33 (m, 2H), 4.27-4.25 (m, 3H), 4.00-3.97 (m, 1H), 3.67-3.58 (m, 2H), 2.68 (s, 3H), 2.46 (s, 3H), 2.21-2.12 (m, 2H), 2.12-2.04 (m, 1H), 2.04-1.94 (m, 4H), 1.94-1.85 (m, 1H), 1.83-1.66 (m, 2H), 1.44-1.29 (m, 2H), 1.25-1.19 (m, 2H), 0.96 (s, 9H).

### Example A276. 2-amino-3-cyano-N-((S)-6-(6-(((1S,4S)-4-(5-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamoyl)pyridin-3-yl)-2,3-dihydrobenzofuran-3-yl)-N-methylquinoline-6-carboxamide

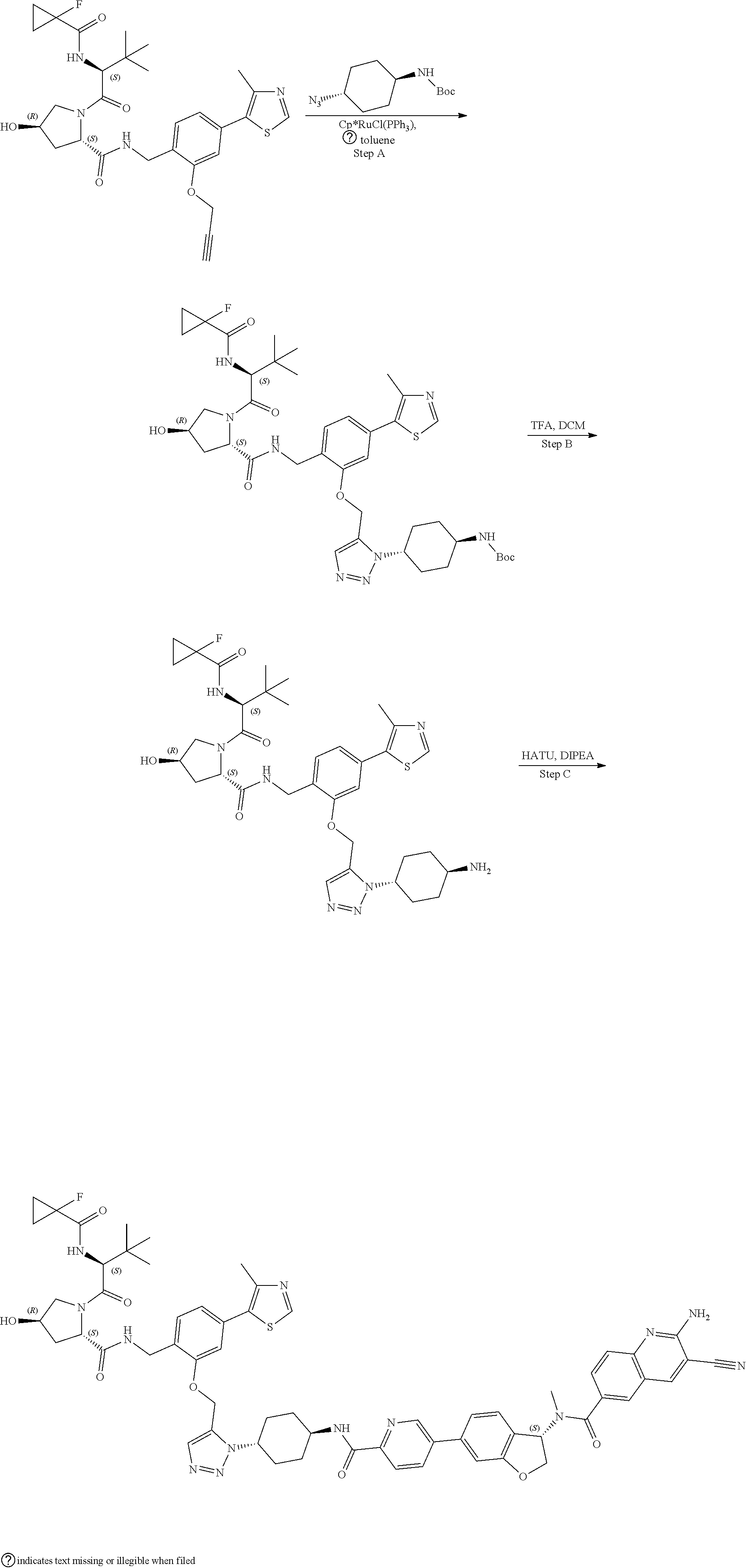

Step A: tert-butyl ((15,4r)-4-(5-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamate

To a solution of (2S,4R)-1-((S)-2-(1-fluorocyclopropane-1-carboxamido)-3,3-dimethylbutanoyl)-4-hydroxy-N-(4-(4-methylthiazol-5-yl)-2-(prop-2-yn-1-yloxy)benzyl)pyrrolidine-2-carboxamide (100 mg, 175 μmol, 1.0 eq.) in Toluene (5 mL) was added tert-butyl ((1r,4r)-4-azidocyclohexyl)carbamate (63 mg, 263 μmol, 1.5 eq.) and 1,3-Cyclopentadiene, 1,2,3,4,5-pentamethyl-, rhodium complex (22 mg, 35.0 μmol, 0.2 eq.). The reaction was stirred at 100° C. for 16 hours. The mixture was poured into water (50 mL), extracted with EtOAc (30 mL×3) and the layers were separated. The organic layer was washed with brine (50 mL), dried over Na₂SO₄, filtered and concentrated. The product was purified by column chromatography (silica gel, 0% MeOH in DCM~10% MeOH in DCM) to afford tert-butyl ((1S,4r)-4-(5-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamate (80 mg, 56% yield) as a brown solid. LC-MS (ESI): mass calcd. For C40H55FN807S, 810.39; m/z found, 811.6 [M+H]⁺.

Steps B-C: 2-amino-3-cyano-N-((S)-6-(6-(((1S,4S)-4-(5-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)methyl)-1H-1,2,3-triazol-1-yl)cyclohexyl)carbamoyl)pyridin-3-yl)-2,3-dihydrobenzofuran-3-yl)-N-methylquinoline-6-carboxamide

Example A276 was synthesized following the same procedures as described in Example A23 (3.2 mg, 7% yield over 2 steps) as a white solid. LC-MS (ESI): mass calcd. for C₆₁H₆₄FN130&S, 1157.47; m/z found, 1158.7 [M+H]⁺. ¹H NMR (400 MHz, DMSO-d₆) δ 9.02 (s, 1H), 8.93 (s, 1H), 8.85 (s, 1H), 8.65-8.54 (m, 2H), 8.29 (d, J=6.7 Hz, 1H), 8.11 (d, J=8.2 Hz, 1H), 7.99 (s, 1H), 7.89-7.82 (m, 2H), 7.61 (d, J=7.8 Hz, 1H), 7.56 (d, J=7.8 Hz, 1H), 7.45 (d, J=7.9 Hz, 1H), 7.39 (d, J=7.2 Hz, 1H), 7.35 (s, 1H), 7.26 (d, J=11.6 Hz, 2H), 7.03 (d, J=7.4 Hz, 1H), 5.47 (s, 2H), 4.84-4.67 (m, 2H), 4.59-4.48 (m, 3H), 4.35-4.17 (m, 4H), 3.63-3.57 (m, 3H), 2.68 (s, 3H), 2.47 (s, 3H), 2.15-2.08 (m, 4H), 2.05-1.98 (m, 3H), 1.91-1.85 (m, 1H), 1.79-1.69 (m, 2H), 1.39-1.29 (m, 2H), 1.24-1.18 (m, 2H), 0.91 (s, 9H).

### Example A278. N-((S)-6-(6-(((18,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N,1-dimethyl-3-(methylamino)-1H-pyrazolo[4,3-c]quinoline-8-carboxamide

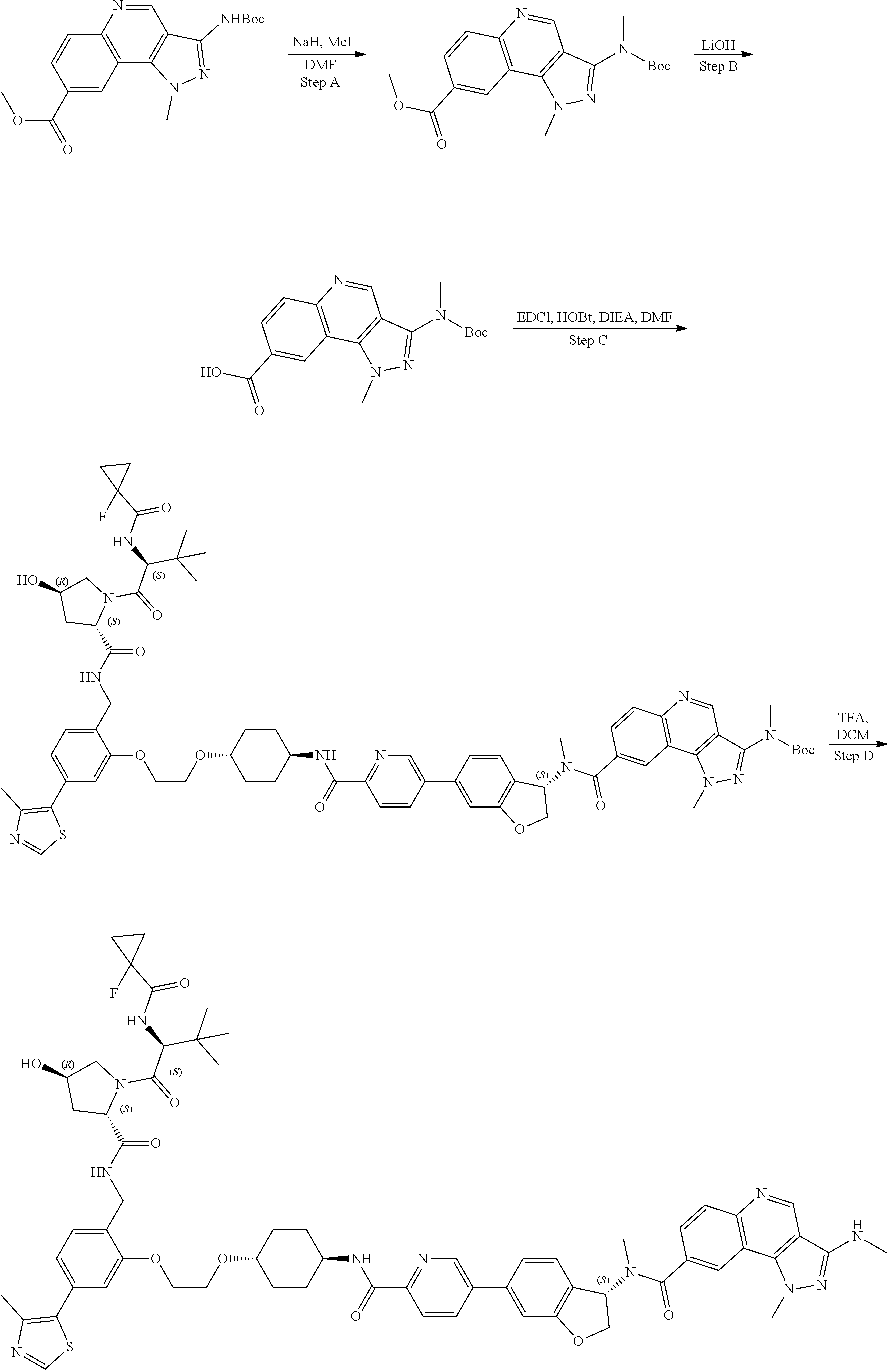

Example A278 was synthesized following the same procedures as described in Example A249 (5.8 mg, 3% yield over 4 steps) as a yellow solid. LC-MS (ESI): mass calcd. for C₆₂H₇₀FN₁₁O₉S, 1163.5/z found, 1166.0 [M+H]⁺. ¹H NMR (400 MHz, DMSO-d₆) δ 9.41 (s, 1H), 8.99 (s, 1H), 8.91 (s, 1H), 8.63 (d, 1H), 8.54-8.42 (m, 2H), 8.32-8.17 (m, 2H), 8.15-8.05 (m, 2H), 7.64 (s, 1H), 7.49-7.25 (m, 5H), 7.05 (s, 1H), 6.97 (d, J=7.7 Hz, 1H), 4.82-4.74 (m, 1H), 4.60 (d, J=9.4 Hz, 1H), 4.52 (t, J=8.1 Hz, 1H), 4.39 (s, 3H), 4.37-4.34 (m, 1H), 4.33-4.28 (m, 1H), 4.27-4.21 (m, 1H), 4.20-4.16 (m, 2H), 3.82-3.80 (m, 2H), 3.68-3.67 (m, 1H), 3.66-. 3.64 (m, 2H), 3.62-3.61 (m, 1H), 3.60-3.58 (m, 1H), 3.39-3.34 (m, 1H), 2.96 (s, 3H), 2.71 (d, J=45.2 Hz, 3H), 2.47 (s, 3H), 2.14-2.08 (m, 1H), 2.07-2.02 (m, 2H), 1.97-1.90 (m, 1H), 1.89-1.82 (m, 2H), 1.56-1.46 (m, 2H), 1.41-1.28 (m, 4H), 1.24-1.19 (m, 2H), 0.96 (s, 9H).

### Example A279. 2-amino-3-cyano-N-((3S)-6-(6-(((1R,3R,5S)-6-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino) bicyclo[3.1.0]hexan-3-yl)carbamoyl)pyridin-3-yl)-2,3-dihydrobenzofuran-3-yl)-N-methylquinoline-6-carboxamide

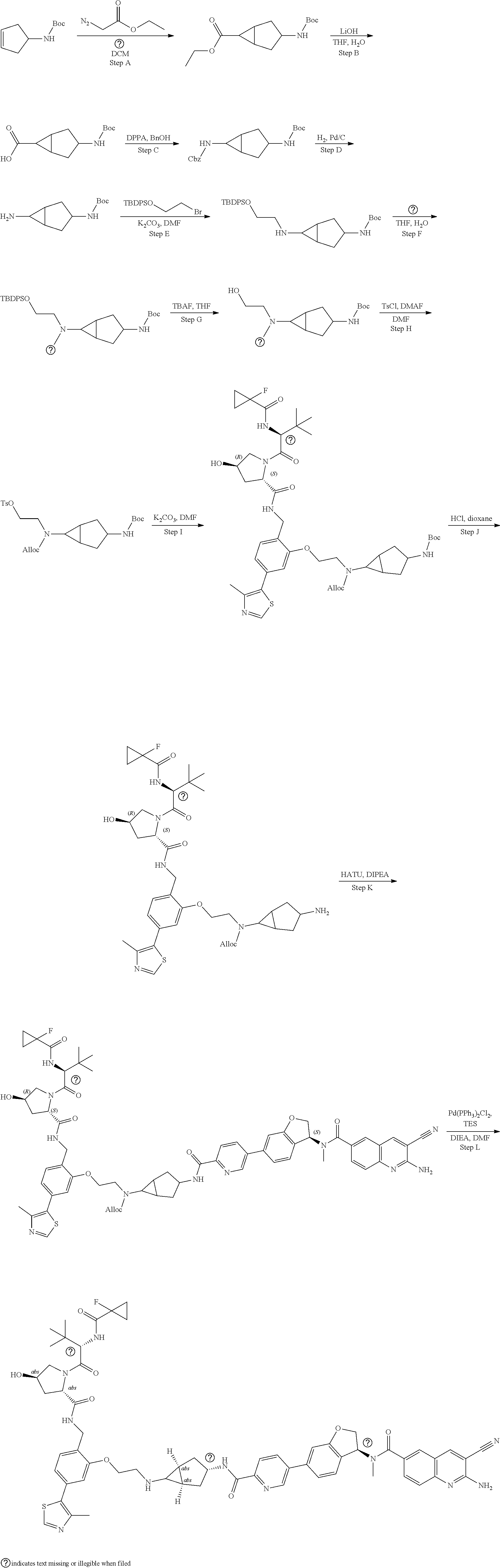

Step A: ethyl 3-((tert-butoxycarbonyl)amino) bicyclo[3.1.0]hexane-6-carboxylate

To a mixture of tert-butyl cyclopent-3-en-1-ylcarbamate (4.0 g, 21.8 mmol, 1.0 eq.) in DCM (60 mL) was added Rhodium (II) AcetateDimer (480 mg, 1.1 mmol, 0.05 eq.) and DAAE (5.0 g, 4.80 mL, 43.7 mmol, 2.0 eq.) at room temperature. The reaction mixture was stirred room temperature for 16 hours. The resulting mixture was quenched with water (80 mL), diluted with DCM (50 mL×3), and the layers were separated. The organic layer was washed with brine (50 mL) three times, dried over anhydrous Na₂SO₄, filtered and concentratedconcentrated under vacuum. The crude product was purified by flash chromatography (100-200 mesh silica gel, 0%~20% EtOAc in PE) to afford ethyl 3-((tert-butoxycarbonyl)amino) bicyclo[3.1.0]hexane-6-carboxylate (1.2 g, 20% yield) as a solid. LC-MS (ESI): mass calcd. For C₁₄H₂₃NO₄, 269.16; m/z found, 270.2 [M+H]⁺.

Step B: 3-((tert-butoxycarbonyl)amino) bicyclo[3.1.0]hexane-6-carboxylic acid

A solution of ethyl 3-((tert-butoxycarbonyl)amino) bicyclo[3.1.0]hexane-6-carboxylate (2.0 g, 7.43 mmol, 1.0 eq.), LiOH (534 mg, 22.3 mmol, 3.0 eq.) in THF (15 mL) and Water (15 mL) was stirred at room temperature. The mixture was stirred at 70° C. for 16 hours. The mixture was neutralized with HCl solution (2 N) to pH=3, extracted with EtOAc (20 mL×3). The combined extracts were dried over anhydrous Na₂SO₄, filtered and evaporated to afford 3-((tert-butoxycarbonyl)amino) bicyclo[3.1.0]hexane-6-carboxylic acid (800 mg, 45% yield) as a white solid, which was used directly without further purification. LC-MS (ESI): mass calcd. For C₁₂H19NO_{4, 241.13}; m/z found, 242.2 [M+H]⁺.

Step C: benzyl tert-butyl bicyclo[3.1.0]hexane-3,6-diyldicarbamate

To a mixture of 3-((tert-butoxycarbonyl)amino) bicyclo[3.1.0]hexane-6-carboxylic acid (450 mg, 1.86 mmol, 1.0 eq.) in Toluene (10 mL) was added TEA (570 mg, 780 μL, 5.59 mmol, 3.0 eq.) and Diphenylphosphoryl azide (620 mg, 483 μL, 2.24 mmol, 1.2 eq.). Then phenylmethanol (400 mg, 3.73 mmol, 2.0 eq.) was added dropwise. This mixture was stirred at 110° C. under N₂ for 3 hours. This mixture was concentrated and purified by column chromatography on silica gel (100-200 mesh silica gel, 0%~40% EtOAc in PE) to give benzyl tert-butyl bicyclo[3.1.0]hexane-3,6-diyldicarbamate (300 mg, 46% yield) as a white solid. LC-MS (ESI): mass calcd. For C₁₉H₂₆N₂O₄, 346.19; m/z found, 347.2 [M+H]⁺.

Step D: tert-butyl (6-aminobicyclo[3.1.0]hexan-3-yl)carbamate

To a solution of benzyl tert-butyl bicyclo[3.1.0]hexane-3,6-diyldicarbamate (100 mg, 289 μmol, 1.0 eq.) in CF₃CH₂OH (8 mL) was added Pd/C (20 mg, 188 μmol, 0.6 eq.). The mixture was stirred at room temperature for 6 hours under H₂. The reaction mixture was filtered and concentrated under reduced pressure to give tert-butyl (6-aminobicyclo[3.1.0]hexan-3-yl)carbamate (50.0 mg, 82% yield) as a yellow oil, which was used directly without further purification. LC-MS (ESI): mass calcd. For C₁₁H₂₀N₂O₂, 212.15; m/z found, 213.2 [M+H]⁺.

Step E: ter-butyl (6-(2-((tert-butyldiphenylsilyl) oxy)ethyl)amino) bicyclo[3.1.0]hexan-3-yl)carbamate

To a solution of tert-butyl (6-aminobicyclo[3.1.0]hexan-3-yl)carbamate (50.0 mg, 235.5 μmol, 1.0 eq.), (2-bromoethoxy)(tert-butyl)diphenylsilane (130 mg, 353.3 μmol, 1.5 eq.) in DMF (6 mL) was added K₂CO₃ (65 mg, 471.1 mol, 2.0 eq.). The mixture was stirred at 80° C. for 16 hours. The resulting solution was diluted with 50 mL of water and extracted with EtOAc (20 mL×3). The combined organic layers were washed with brine (20 mL), dried over anhydrous Na₂SO₄ and concentrated under reduced pressure. The residue was purified by flash column chromatography on silica gel (100-200 mesh silica gel, 0-8% MeOH in DCM) to give tert-butyl (6-((2-((tert-butyldiphenylsilyl)oxy)ethyl)amino) bicyclo[3.1.0]hexan-3-yl)carbamate (80 mg, 69% yield) as a solid. LC-MS (ESI): mass calcd. For C29H42N₂O₃Si, 494.30; m/z found, 495.4 [M+H]⁺

Step F: allyl (3-((tert-butoxycarbonyl)amino) bicyclo[3. 1.0]hexan-6-yl)(2-((tert-butyldiphenylsilyl)oxy)ethyl)carbamate

To a mixture of tert-butyl (6-((2-((tert-butyldiphenylsilyl)oxy)ethyl)amino) bicyclo[3.1.0]hexan-3-yl)carbamate (60.0 mg, 121 μmol, 1.0 eq.), Sodium bicarbonate (20.0 mg, 243 μmol, 2.0 eq.) in THF (2 mL) and Water (2 mL) was added chloro (prop-2-en-1-yloxy) methanone (18 mg, 15.5 μL, 146 μmol, 1.2 eq.). The reaction mixture was purged with nitrogen three times, then heated to room temperature for 2 hours under nitrogen. The resulting solution was diluted with 50 mL of water and extracted with EtOAc (30 mL×3). The combined organic layers were washed with brine (20 mL), dried over anhydrous Na₂SO₄ and concentrated under reduced pressure. The crude product was purified by flash silica gel chromatography, elution with a gradient of 0 to 10% MeOH in DCM to give allyl (3-((tert-butoxycarbonyl)amino) bicyclo[3.1.0]hexan-6-yl)(2-((tert-butyldiphenylsilyl)oxy)ethyl)carbamate (50 mg, 71% yield) as a solid. LC-MS (ESI): mass calcd. For C₃₃H₄₆N₂O₅Si, 578.32; m/z found, 579.4 [M+H]⁺.

Step G: allyl (3-((tert-butoxycarbonyl)amino) bicyclo[3. 1.0]hexan-6-yl)(2-hydroxyethyl)carbamate

A solution of allyl (3-((tert-butoxycarbonyl)amino) bicyclo[3.1.0]hexan-6-yl)(2-((tert-butyldiphenylsilyl)oxy)ethyl)carbamate (60.0 mg, 104 μmol, 1.0 eq.), TBAF (54.0 mg, 207 μmol, 2.0 eq.) in THF (3 mL) with stirring at room temperature. The mixture was stirred at room temperature for 2 hours. The resulting solution was diluted with 50 mL of water and extracted with EtOAc (30 mL×3). The combined organic layers were washed with brine (20 mL), dried over anhydrous Na₂SO₄ and concentrated under reduced pressure. The crude product was purified by flash silica gel chromatography, elution with a gradient of 0 to 10% MeOH in DCM to afford allyl (3-((tert-butoxycarbonyl)amino) bicyclo[3.1.0]hexan-6-yl)(2-hydroxyethyl)carbamate (30 mg, 85% yield) as a white solid. LC-MS (ESI): mass calcd. For C₁₇H₂₈N₂O₅, 340.20; m/z found, 341.3 [M+H]⁺.

Step H: 2-(((allyloxy)carbonyl)(3-((tert-butoxycarbonyl)amino) bicyclo[3.1.0]hexan-6-yl)amino)ethyl 4-methylbenzenesulfonate

To a stirred mixture of allyl (3-((tert-butoxycarbonyl)amino) bicyclo[3.1.0]hexan-6-yl)(2-hydroxyethyl)carbamate (30.0 mg, 88.1 μmol, 1.0 eq.) in DCM (4 mL) were added TEA (23 mg, 30.7 μL, 220 μmol, 2.5 eq.), Ts-Cl (20.0 mg, 106 μmol, 1.2 eq.). DMAP (2.0 mg, 17.6 μmol, 0.2 eq.) was then added and the mixture was stirred at room temperature for 3 hours. The resulting solution was diluted with 50 mL of water and extracted with EtOAc (30 mL×3). The combined organic layers were washed with brine (20 mL), dried over anhydrous Na₂SO₄ and concentrated under reduced pressure. The crude product was purified by flash silica chromatography, eluting with a gradient of 0~3% MeOH in DCM to afford 2-(((allyloxy)carbonyl)(3-((tert-butoxycarbonyl)amino) bicyclo[3.1.0]hexan-6-yl)amino)ethyl 4-methylbenzenesulfonate (35 mg, 80% yield) as a white solid. LC-MS (ESI): mass calcd. For C₂₄H₃₄N207S, 494.21; m/z found, 495.3 [M+H]⁺.

Steps 1-K: allyl (3-(5-((S)-3-(2-amino-3-cyano-N-methylquinoline-6-carboxamido)-2,3-dihydrobenzofuran-6-yl)picolinamido) bicyclo[3.1.0]hexan-6-yl)(2-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)carbamate

Allyl (3-(5-((S)-3-(2-amino-3-cyano-N-methylquinoline-6-carboxamido)-2,3-dihydrobenzofuran-6-yl)picolinamido) bicyclo[3.1.0]hexan-6-yl)(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)carbamate was synthesized following the same procedures as described in Example A23.

Step L: 2-amino-3-cyano-N-((3S)-6-(6-(((IR, 3R,5S)-6-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino) bicyclo[3.1.0]hexan-3-yl)carbamoyl)pyridin-3-yl)-2,3-dihydrobenzofuran-3-yl)-N-methylquinoline-6-carboxamide

To a stirred mixture of allyl (3-(5-((S)-3-(2-amino-3-cyano-N-methylquinoline-6-carboxamido)-2,3-dihydrobenzofuran-6-yl)picolinamido) bicyclo[3.1.0]hexan-6-yl)(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)carbamate (20.0 mg, 16.6 μmol, 1.0 eq.) in DMF (2.00 mL) were added Bis-(triphenylphosphino)-palladous chloride (1.0 mg, 1.66 μmol, 0.1 eq.), Meldrum's acid (5.0 mg, 33.3 μmol, 2.0 eq.) and DIEA (4.30 mg, 5.79 μL, 33.3 μmol, 2.0 eq.). Triethylsilane (3.87 mg, 5.31 μL, 33.3 μmol, 2.0 eq.) was then added and the mixture was stirred at room temperature for 1 hour. The residue was purified by prep-HPLC (C18, 20%-95% MeCN in water with 0.1% TFA) to give 2-amino-3-cyano-N-((3S)-6-(6-(((1R,3R,5S)-6-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino) bicyclo[3.1.0]hexan-3-yl)carbamoyl)pyridin-3-yl)-2,3-dihydrobenzofuran-3-yl)-N-methylquinoline-6-carboxamide (2 mg, 11% yield) as white solid.

LC-MS (ESI): mass calcd, for C₆₀H₆₄FN₁₁O₈S, 1117.46; m/z found, 1118.8 [M+H]⁺. ¹HNMR (400 MHz, DMSO-d₆) δ 8.99 (s, 1H), 8.85-8.79 (m, 3H), 8.61-8.54 (m, 2H), 8.28-8.25 (m, 1H), 8.05 (d, J=8.1 Hz, 1H), 7.95 (s, 1H), 7.81-7.78 (m, 1H), 7.60-7.53 (m, 2H), 7.41 (d, J=7.8 Hz, 1H), 7.36 (d, J=7.7 Hz, 1H), 7.32-7.26 (m, 3H), 7.06-7.04 (m, 1H), 7.02 (d, J=7.7 Hz, 1H), 4.85-4.64 (m, 2H), 4.58 (d, J=9.3 Hz, 1H), 4.50-4.40 (m, 3H), 4.37-4.29 (m, 4H), 3.67-3.56 (m, 5H), 2.74-2.70 (m, 1H), 2.67 (s, 3H), 2.45 (s, 3H), 2.38-2.33 (m, 2H), 2.10-2.05 (m, 1H), 1.91-1.83 (m, 5H), 1.40-1.32 (m, 2H), 1.24-1.20 (m, 2H), 0.93 (s, 9H).

### Example A280. 2-amino-3-cyano-N-((3S)-6-(6-(((1R,3S,5S)-6-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)amino) bicyclo[3.1.0]hexan-3-yl)carbamoyl)pyridin-3-yl)-2,3-dihydrobenzofuran-3-yl)-N-methylquinoline-6-carboxamide

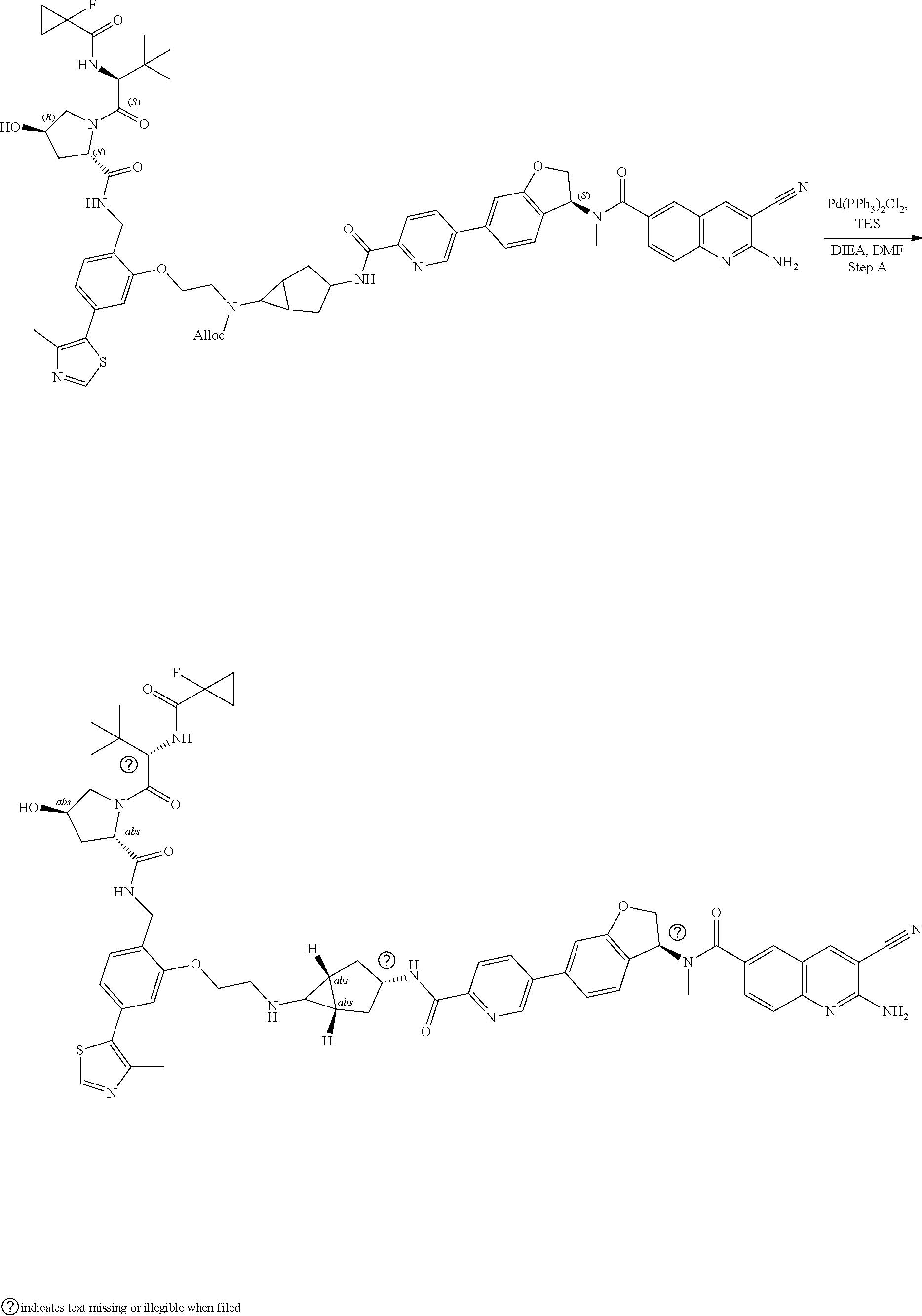

Example A280 was synthesized following the same procedures as described in Example 279 (1.0 mg, 5% yield) as a white solid. LC-MS (ESI): mass calcd, for C₆₀H₆₄FN₁₁O₈S, 1117.46; m/z found, 1118.7 [M+H]⁺. ¹H NMR (400 MHz, DMSO-d₆) δ 9.01 (s, 1H), 8.94-8.91 (m, 1H), 8.81-8.73 (m, 4H), 8.58-8.53 (m, 1H), 8.29-8.25 (m, 1H), 8.06 (d, J=8.4 Hz, 1H), 7.95 (s, 1H), 7.81-7.77 (m, 1H), 7.60-7.53 (m, 2H), 7.44-7.37 (m, 2H), 7.34 (s, 1H), 7.31-7.27 (m, 2H), 7.07 (s, 1H), 7.05-7.02 (m, 1H), 4.88-4.65 (m, 2H), 4.60 (d, J=9.4 Hz, 1H), 4.54-4.48 (m, 1H), 4.46-4.40 (m, 1H), 4.38-4.30 (m, 4H), 4.16-4.05 (m, 1H), 3.70-3.55 (m, 5H), 2.94-2.91 (m, 1H), 2.67 (s, 3H), 2.47 (s, 3H), 2.15-2.11 (m, 3H), 1.98-1.91 (m, 3H), 1.80-1.78 (m, 2H), 1.40-1.33 (m, 2H), 1.24-1.20 (m, 2H), 0.95 (s, 9H).

### Example A281. N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-2-(isopropylamino)-N,3-dimethylquinoline-6-carboxamide

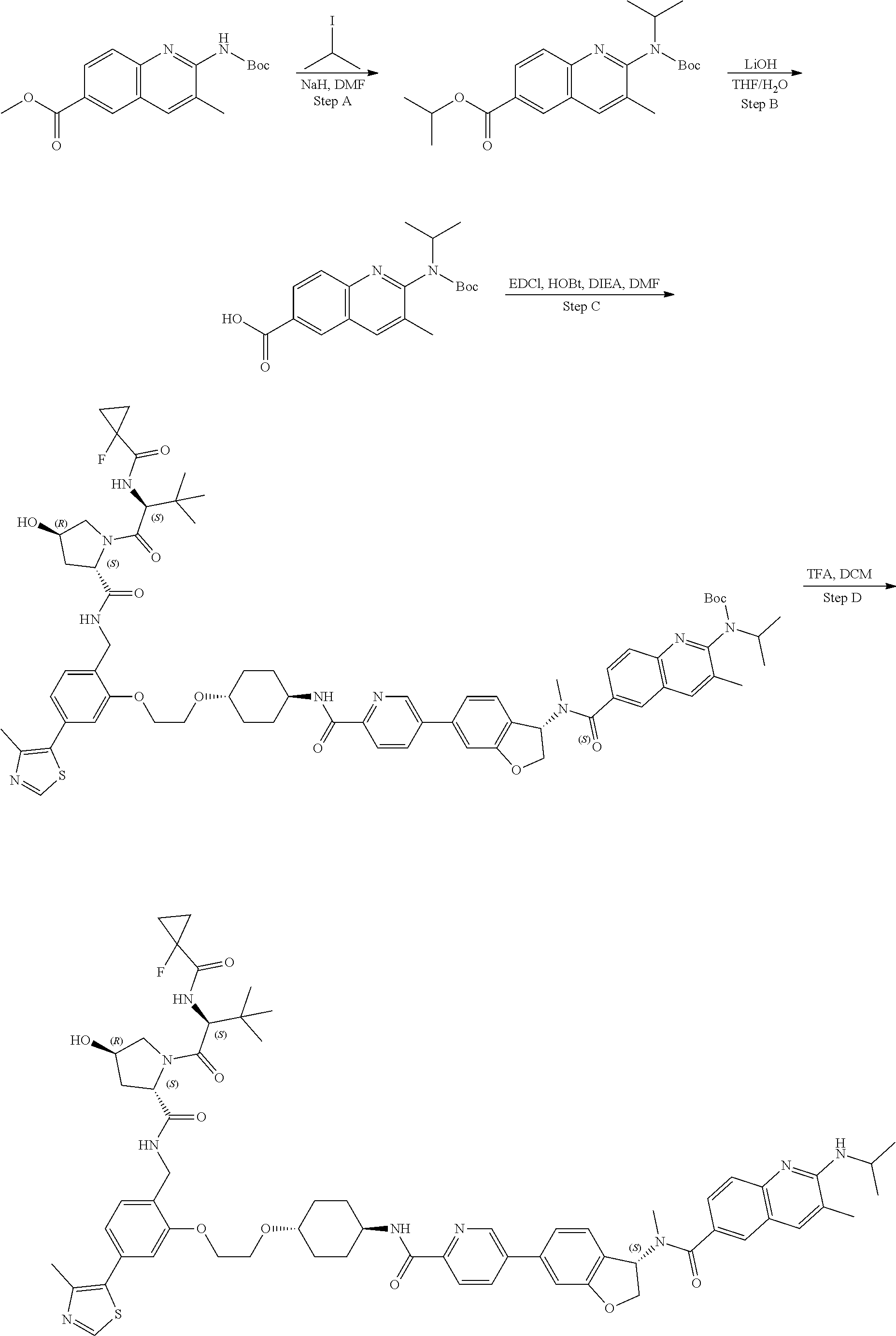

Example A281 was synthesized following the same procedures as described in Example A249 (1.0 mg, 10% yield over 4 steps) as a white solid. LC-MS (ESI): mass calcd. For C₆₃H₇₄FN₉O₉S, 1151.53; m/z found, 1152.53 [M+H]⁺. ¹H NMR (400 MHz, DMSO-d₆) δ 8.99 (s, 1H), 8.92 (s, 1H), 8.51-8.47 (m, 2H), 8.29-8.21 (m, 2H), 8.09 (d, J=8.2 Hz, 1H), 8.01 (s, 2H), 7.86 (d, J=8.4 Hz, 1H), 7.56 (d, J=7.8 Hz, 1H), 7.41 (d, J=7.8 Hz, 2H), 7.35 (s, 1H), 7.29 (dd, J=9.2, 2.3 Hz, 1H), 7.06 (s, 1H), 6.97 (d, J=7.8 Hz, 1H), 4.81 (s, 1H), 4.70 (d, J=8.9 Hz, 1H), 4.60 (d, J=9.0 Hz, 1H), 4.52 (t, J=8.2 Hz, 1H), 4.48-4.39 (m, 1H), 4.36 (s, 1H), 4.30 (d, J=5.6 Hz, 1H), 4.27-4.21 (m, 1H), 4.18 (s, 2H), 3.81 (s, 4H), 3.68-3.61 (m, 3H), 2.67 (s, 3H), 2.47 (s, 3H), 2.35 (s, 3H), 2.09-2.02 (m, 3H), 1.97-1.92 (m, 1H), 1.89-1.82 (m, 2H), 1.56-1.47 (m, 2H), 1.36 (d, J=6.4 Hz, 6H), 1.34-1.28 (m, 2H), 1.25-1.18 (m, 4H), 0.96 (s, 9H).

### Example A282. 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-3-methoxy-N-methylquinoline-6-carboxamide

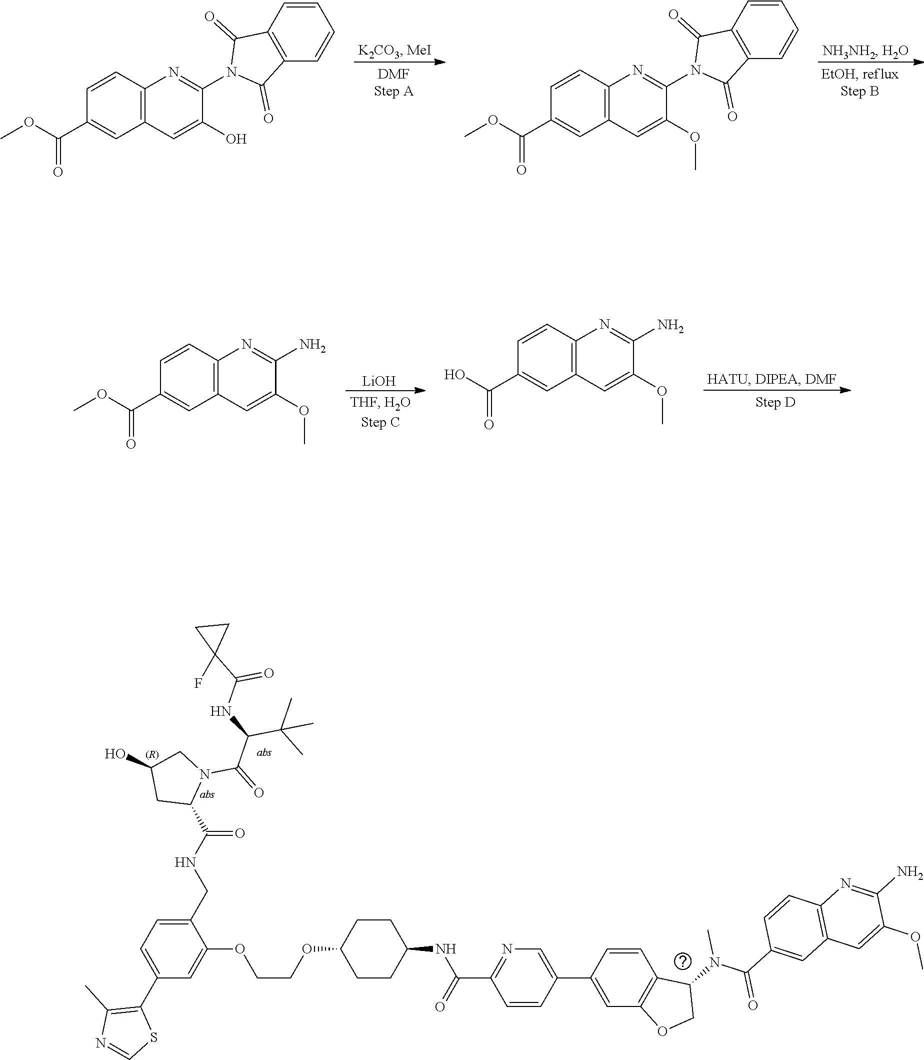

Example A282 was synthesized following the same procedures as described in Example A221 (4.3 mg, 2% yield over 4 steps) as a white solid. LC-MS (ESI): mass calcd. for C₆₀H₆₈FN₉O₁₀S, 1125.5/z found, 1126.5 [M+H]⁺. ¹H NMR (400 MHz, DMSO-d₆) δ 8.99 (s, 1H), 8.94-8.89 (m, 2H), 8.52-8.44 (m, 2H), 8.27 (d, J=8.1 Hz, 1H), 8.09 (d, J=8.1 Hz, 1H), 8.00 (s, 1H), 7.89 (s, 1H), 7.73 (s, 2H), 7.56 (d, J=7.7 Hz, 1H), 7.41 (d, J=7.7 Hz, 2H), 7.35 (s, 1H), 7.29 (d, J=6.5 Hz, 1H), 7.05 (s, 1H), 6.97 (d, J=7.7 Hz, 1H), 4.84-4.65 (m, 2H), 4.60 (d, J=9.0 Hz, 1H), 4.51 (t, J=8.0 Hz, 1H), 4.38-4.32 (m, 1H), 4.27 (dd, J=19.5, 5.9 Hz, 2H), 4.20-4.14 (m, 2H), 4.04 (s, 3H), 3.85-3.77 (m, 4H), 3.42-3.34 (m, 3H), 2.67 (s, 3H), 2.47 (s, 3H), 2.07-2.02 (m, 2H), 2.01-1.92 (m, 2H), 1.90-1.82 (m, 2H), 1.57-1.48 (m, 2H), 1.43-1.30 (m, 6H), 0.96 (s, 9H)

### Example A283. 2-amino-3-ethynyl-N-((S)-6-(6-((1-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperidin-4-yl)carbamoyl) pyridin-3-yl)-2,3-dihydrobenzofuran-3-yl)-N-methylquinoline-6-carboxamide

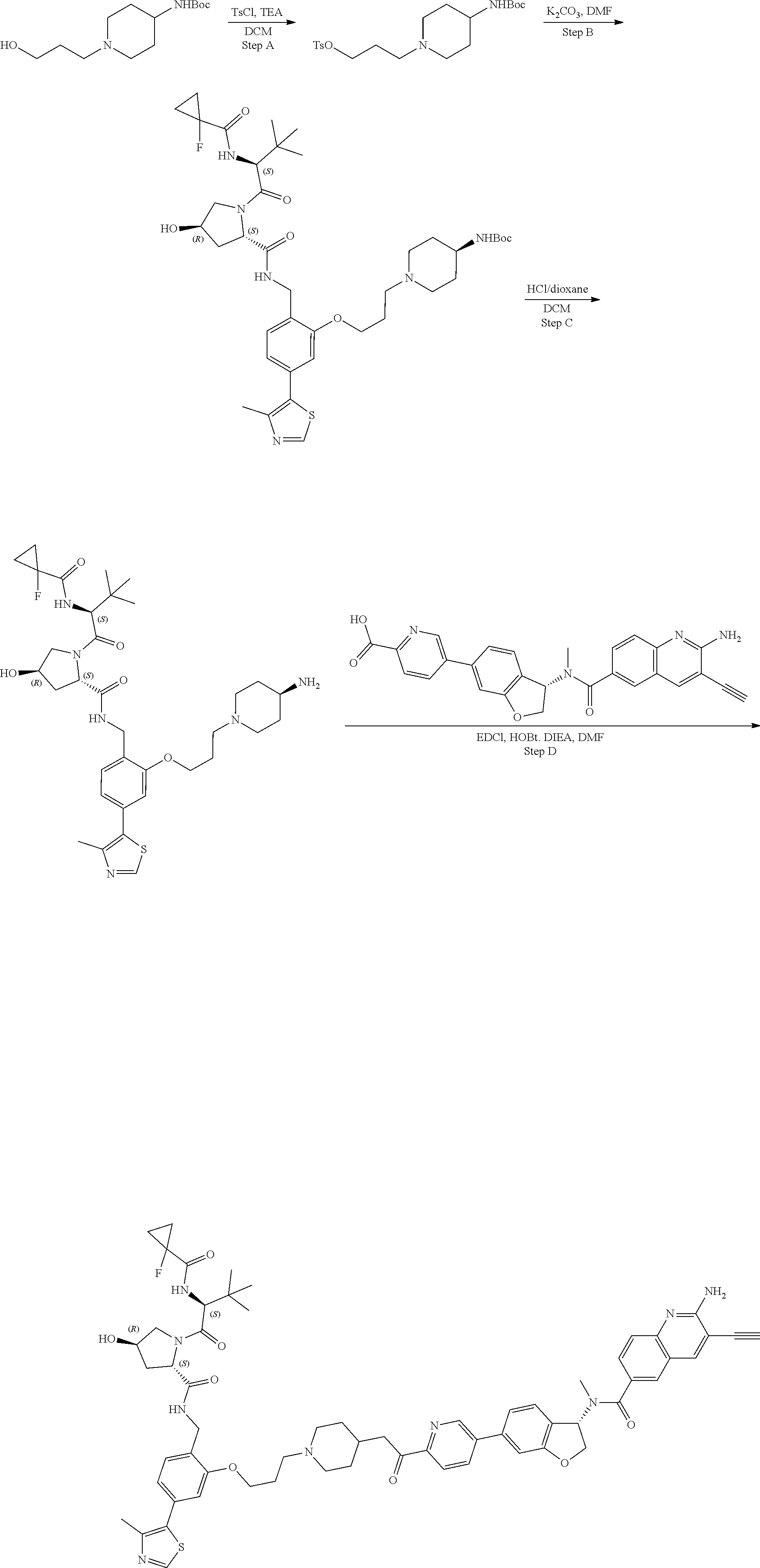

Example A283 was synthesized following the same procedures as described in Example A23 (24.0 mg, 46% yield over 4 steps) as a white solid. LC-MS (ESI): mass calcd. For C₆₁H₆₇FN₁₀O₈S, 1118.48; m/z found, 1119.48 [M+H]⁺. ¹H NMR (400 MHz, DMSO-d₆) δ 9.23 (s, 1H), 9.03-8.89 (m, 3H), 8.62-8.48 (m, 2H), 8.30 (d, J=7.8 Hz, 1H), 8.10 (d, J=8.3 Hz, 1H), 8.01 (s, 1H), 7.84 (d, J=9.1 Hz, 1H), 7.69 (s, 1H), 7.57 (d, J=7.9 Hz, 1H), 7.46-7.38 (m, 2H), 7.36 (s, 1H), 7.29 (d, J=7.5 Hz, 1H), 7.04-6.98 (m, 2H), 4.87 (s, 1H), 4.87 (s, 1H), 4.74-4.67 (m, 1H), 4.60 (d, J=9.4 Hz, 1H), 4.53 (t, J=8.2 Hz, 1H), 4.36 (s, 1H), 4.30 (d, J=5.4 Hz, 2H), 4.19-4.07 (m, 3H), 3.67-3.59 (m, 4H), 3.42 (s, 2H), 3.30 (s, 2H), 3.20-3.11 (m, 2H), 2.67 (s, 3H), 2.47 (s, 3H), 2.21-2.15 (m, 2H), 2.10-2.00 (m, 3H), 1,98-1.87 (m, 3H), 1.41-1.31 (m, 2H), 1.24-1.18 (m, 2H), 0.95 (s, 9H).

### Example A284. 2-amino-3-ethynyl-N-((S)-6-(6-((3-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)benzyl)carbamoyl)pyridin-3-yl)-2,3-dihydrobenzofuran-3-yl)-N-methylquinoline-6-carboxamide

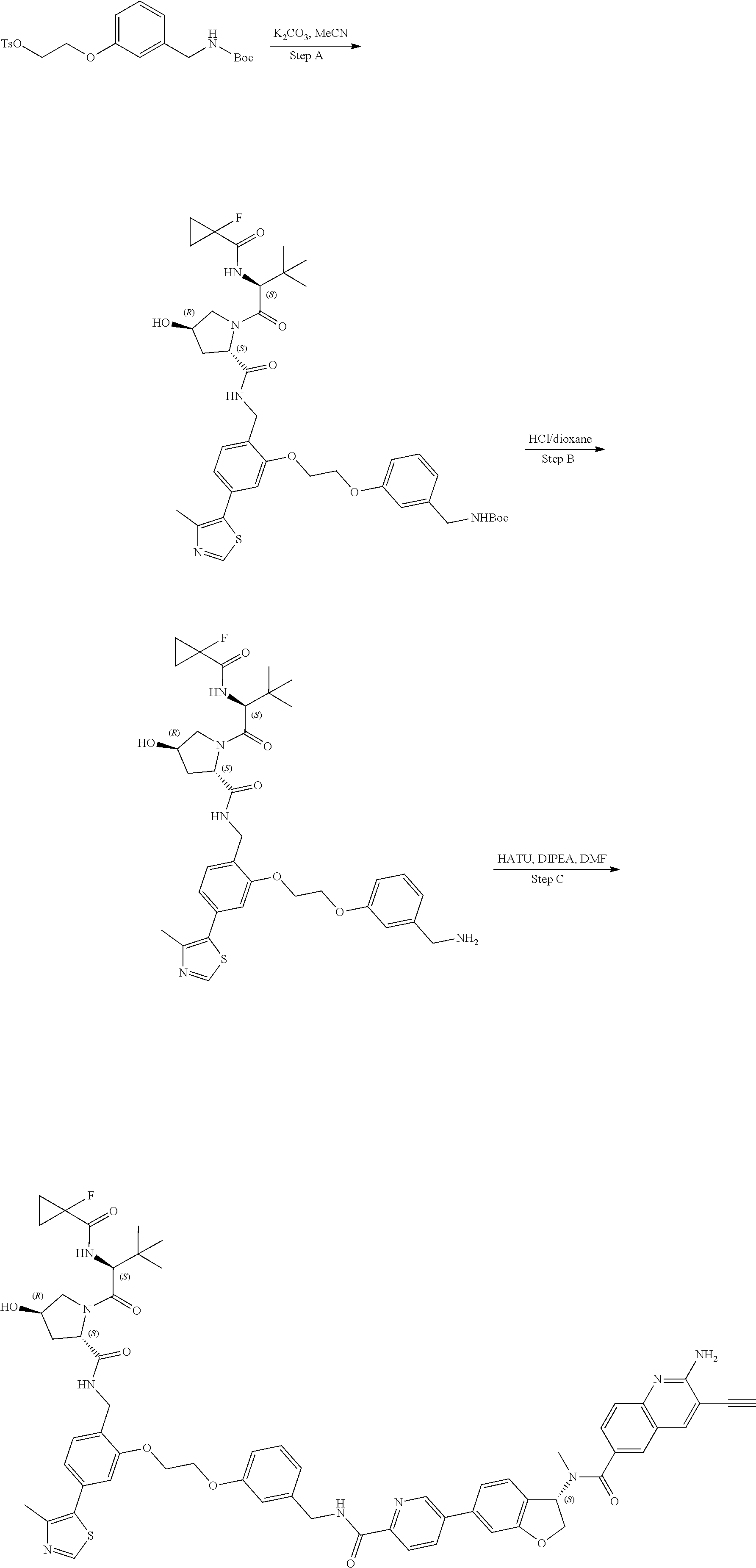

Example A284 was synthesized following the same procedures as described in Example A234 (12.0 mg, 24% yield over 3 steps) as a white solid. LC-MS (ESI): mass calcd. For C₆₂H₆₂FN₉O₉S, 1127.44/z found, 1128.9 [M+H]⁺. ¹H NMR (400 MHz, DMSO-d₆) δ 9.34 (t, J=6.3 Hz, 1H), 8.98 (s, 1H), 8.95 (s, 1H), 8.69-8.62 (m, 1H), 8.53-8.39 (m, 2H), 8.30-8.26 (m, 1H), 8.11 (d, J=8.2 Hz, 1H), 8.06 (s, 1H), 7.89 (d, J=8.5 Hz, 1H), 7.77-7.68 (m, 1H), 7.56 (d, J=7.7 Hz, 1H), 7.45-7.38 (m, 2H), 7.36 (s, 1H), 7.31-7.23 (m, 2H), 7.11-7.07 (m, 1H), 7.00-6.96 (m, 2H), 6.93 (d, J=7.5 Hz, 1H), 6.91-6.87 (m, 1H), 4.93 (s, 1H), 4.88-4.63 (m, 3H), 4.59 (d, J=9.2 Hz, 1H), 4.53-4.48 (m, 3H), 4.42-4.39 (m, 2H), 4.36-4.32 (m, 3H), 4.29-4.25 (m, 1H), 4.21-4.16 (m, 1H), 3.62-3.60 (m, 2H), 2.67 (s, 3H), 2.45 (s, 3H), 2.10-2.05 (m, 1H), 1.95-1.86 (m, 1H), 1.43-1.30 (m, 2H), 1.25-1.19 (m, 2H), 0.94 (s, 9H).

### Example A285. 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-3-(1-fluorocyclopropyl)-N-methylquinoline-6-carboxamide

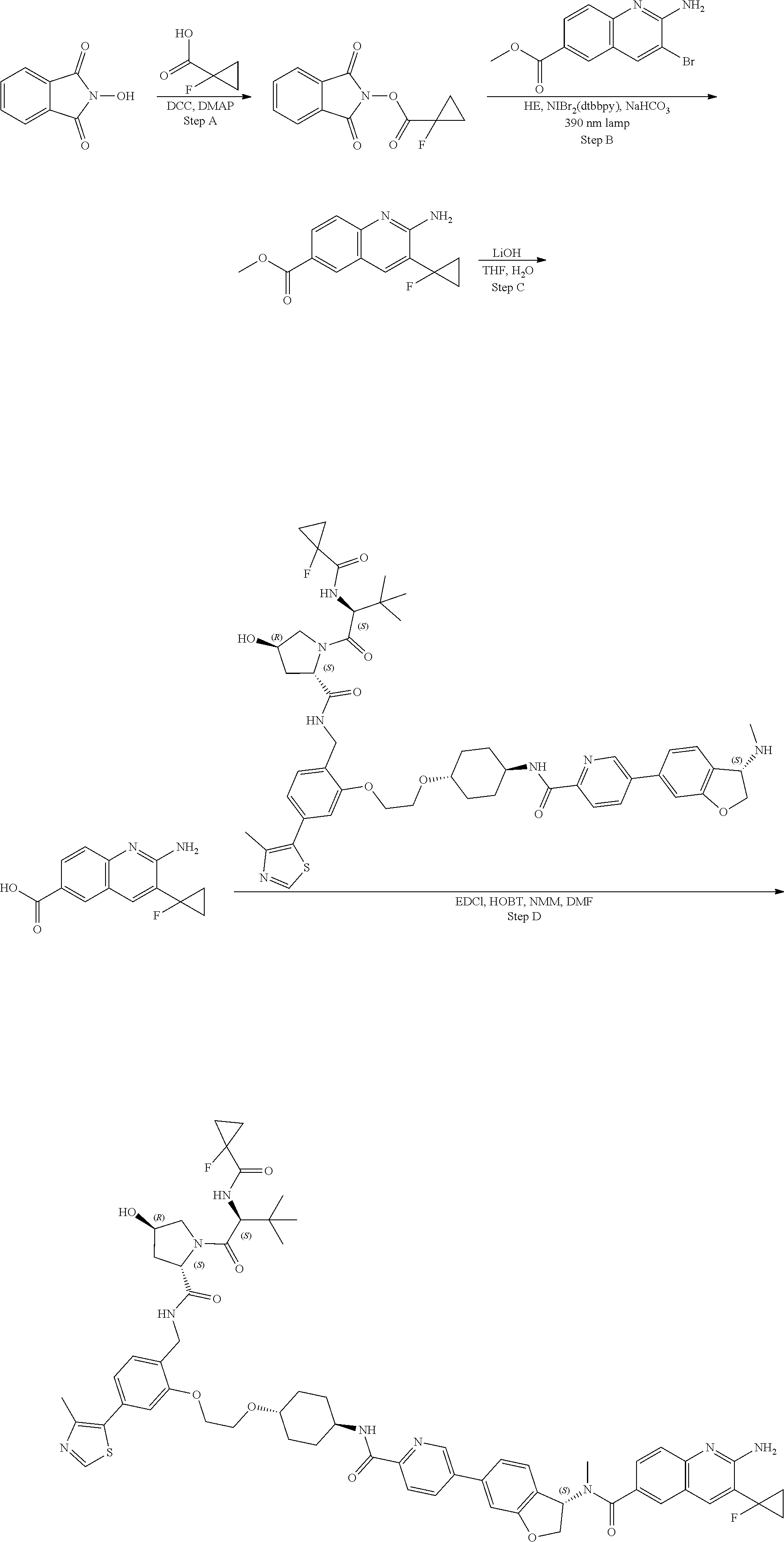

Example A285 was synthesized following the same procedures as described in Example A170 (1 mg, 1% yield over 4 steps) as a white solid. LC-MS (ESI): mass calcd. for C₆₂H69F₂NO₉S, 1153.5/z found, 1155.1 [M+H]⁺. ¹H NMR (400 MHz, DMSO-d₆) δ 8.99 (s, 1H), 8.92 (s, 1H), 8.52-8.45 (m, 2H), 8.27 (s, 2H), 8.08 (d, J=8.1 Hz, 1H), 7.95 (d, J=8.7 Hz, 1H), 7.80 (s, 1H), 7.56 (s, 1H), 7.41 (d, J=7.7 Hz, 2H), 7.35 (s, 1H), 7.29 (d, J=7.0 Hz, 1H), 7.28-7.16 (m, 1H), 7.11 (s, 1H), 7.05 (s, 1H), 6.97 (d, J=9.1 Hz, 1H), 5.37-5.29 (m, 1H), 4.78-4.67 (m, 1H), 4.60 (d, J=9.0 Hz, 1H), 4.55-4.48 (m, 1H), 4.37-4.34 (m, 1H), 4.33-4.28 (m, 1H), 4.27-4.21 (m, 1H), 4.20-4.15 (m, 2H), 3.87-3.73 (m, 3H), 3.70-3.55 (m, 2H), 3.52-3.45 (m, 3H), 2.67 (s, 3H), 2.47 (s, 3H), 2.12-2.09 (m, 1H), 2.03-1.97 (m, 3H), 1.96-1.90 (m, 2H), 1.88-1.83 (m, 2H), 1.74-1.66 (m, 2H), 1.55-1.42 (m, 4H), 1.40-1.31 (m, 4H), 0.96 (s, 9H).

### Example A286. 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-3-(3,3,3-trifluoroprop-1-en-2-yl)quinoline-6-carboxamide

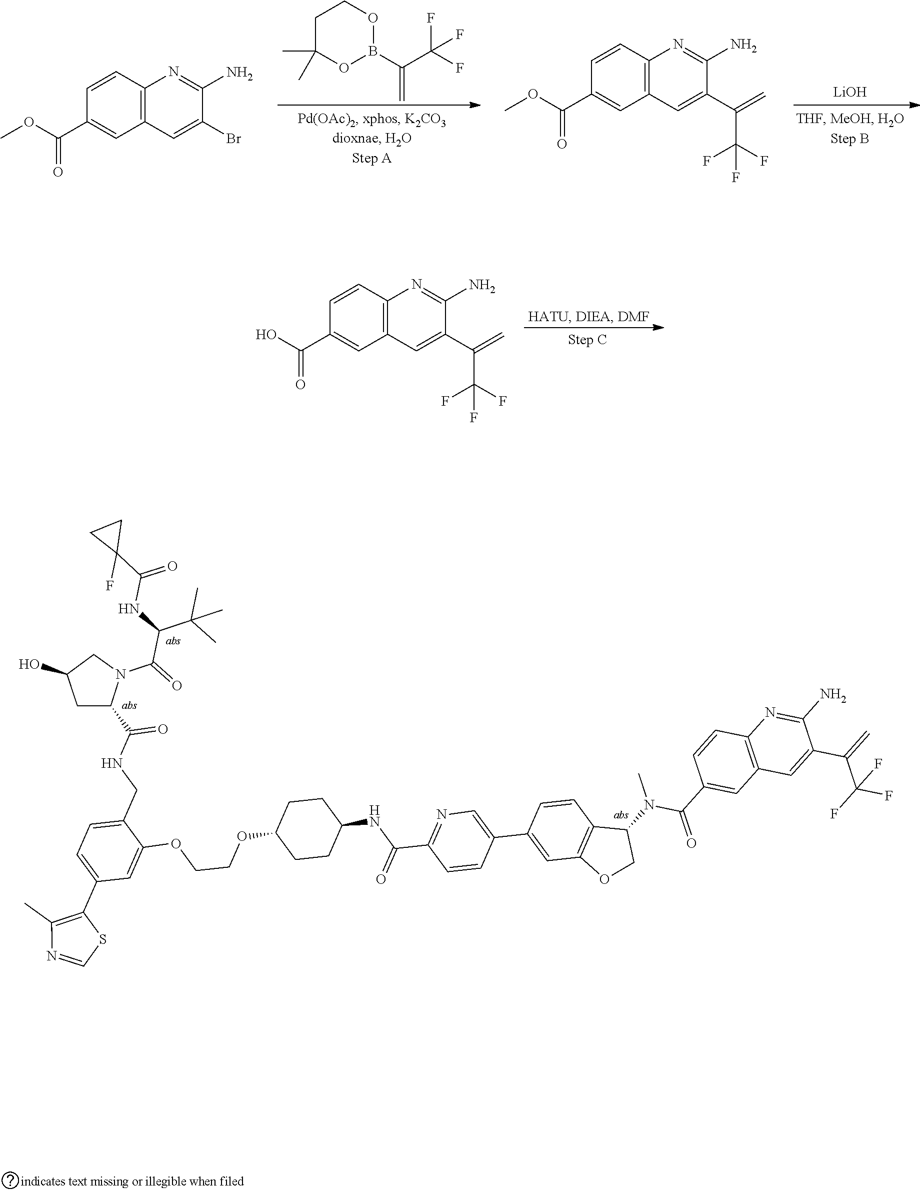

Example A286 was synthesized following the same procedures as described in Example A271 (3.0 mg, 3% yield over 3 steps) as a white solid. LC-MS (ESI): mass calcd. for C₆₂H₆₇F₄N₉O₉S, 1189.47; m/z found, 1190.9 [M+H]⁺. ¹H NMR (400 MHz, DMSO-d₆) δ 8.98 (s, 1H), 8.92 (s, 1H), 8.49 (d, J=7.3 Hz, 2H), 8.27 (d, J=7.8 Hz, 1H), 8.08 (d, J=8.2 Hz, 1H), 7.98 (s, 1H), 7.93 (s, 1H), 7.66 (d, J=8.8 Hz, 1H), 7.54 (t, J=7.1 Hz, 2H), 7.39 (t, J=9.0 Hz, 2H), 7.34-7.27 (m, 2H), 7.05 (s, 1H), 6.97 (d, J=7.7 Hz, 1H), 6.56 (s, 2H), 6.36 (s, 1H), 5.99 (s, 1H), 5.19 (s, 1H), 4.76 (s, 1H), 4.69-4.66 (m, 1H), 4.59 (d, J=9.4 Hz, 1H), 4.52 (t, J=7.8 Hz, 1H), 4.39-4.32 (m, 2H), 4.29 (d, J=6.1 Hz, 1H), 4.24 (d, J=5.4 Hz, 1H), 4.18-4.15 (m, 2H), 3.82-3.78 (m, 4H), 3.65-3.63 (m, 2H), 2.67 (s, 3H), 2.46 (s, 3H), 2.09-1.83 (m, 6H), 1.51 (d, J=12.3 Hz, 2H), 1.40-1.28 (m, 4H), 1.21 (dd, J=7.8, 2.7 Hz, 2H), 0.95 (s, 9H).

### Example A287. 2-amino-3-ethynyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)-N-methylquinoline-6-carboxamide

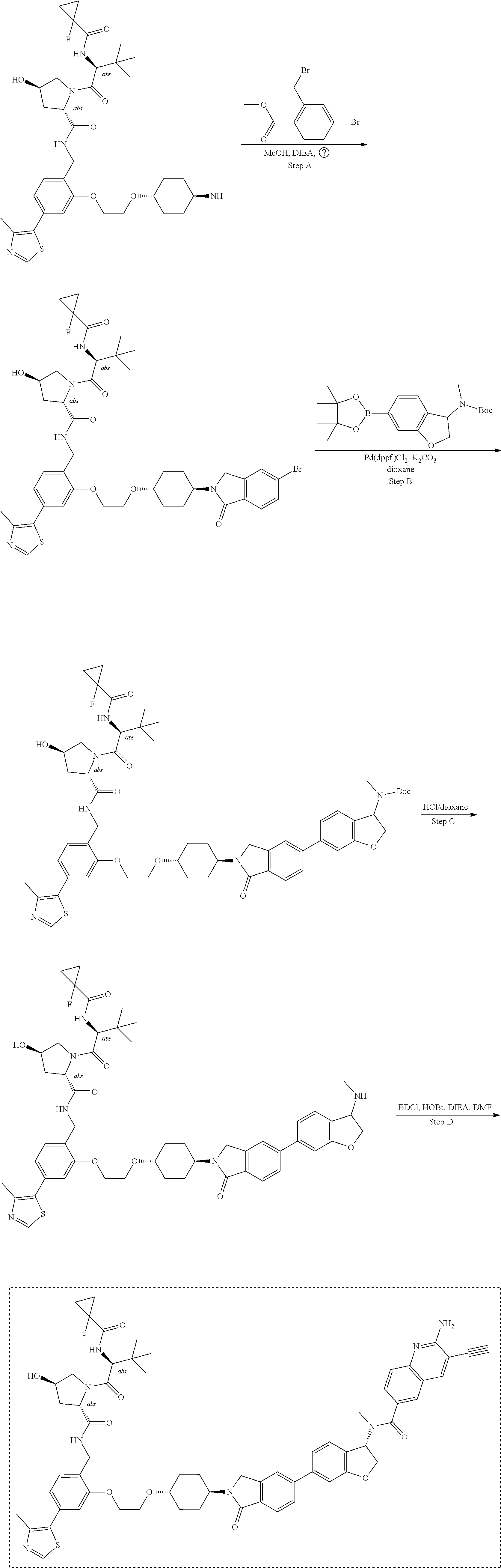

Step A: (2S,4R)-N-(2-(2-(((1r,4S)-4-(5-bromo-1-oxoisoindolin-2-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of (2S,4R)-N-(2-(2-(((1r,4S)-4-aminocyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (50 mg, 74.2 μmol, 1.0 eq.) in MeCN (10 mL) was added DIEA (25.9 μL, 148 μmol, 2.0 eq.) and methyl methyl 4-bromo-2-(bromomethyl)benzoate (45.7 mg, 148 μmol, 2.0 eq.) under N₂. The mixture was stirred at 80° C. for 4 hours. The reaction mixture was quenched with water (30 mL), extracted with EtOAc (30 mL×3). The combined organic layers were washed with brine three times, dried over anhydrous Na₂SO₄ and concentrated. The residue was purified by column chromatography (silica gel, 0% EtOAc in PE~100% EtOAc in PE) to afford (2S,4R)-N-(2-(2-(((1r,4S)-4-(5-bromo-1-oxoisoindolin-2-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (50 mg, 74% yield) as a yellow solid. LC-MS (ESI): mass calcd. For C₄₂H₅₁BrFN₅O₇S, 868.9; m/z found, 868.3/870.3 [M+H]⁺.

Step B: tert-butyl ((S)-6-(2-((1S, 4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)(methyl)carbamate

To a solution of (2S,4R)-N-(2-(2-(((1r,4S)-4-(5-bromo-1-oxoisoindolin-2-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (50 mg, 57.5 μmol, 1.0 eq.) in 1,4-Dioxane (2 mL) and Water (0.1 mL) was added Pd (dppf) C12 (4.21 mg, 5.75 μmol, 0.1 eq.), tert-butyl(S)-methyl (6-(4,4,5,5-tetramethyl-1,3,2-dioxaborolan-2-yl)-2,3-dihydrobenzofuran-3-yl)carbamate (25.9 mg, 69.1 μmol, 1.2 eq.) and K₂CO₃ (23.9 mg, 173 μmol, 3.0 eq.) under N₂. The mixture was stirred at 100 for 2 hours. The reaction mixture was quenched with water (30 mL), and then extracted with EtOAc (30 mL×2). The combined organic layers were washed with brine three times, dried over anhydrous Na₂SO₄ and concentrated. The residue was purified by column chromatography (silica gel, 0% EtOAc in PE~100% EtOAc in PE) to afford tert-butyl ((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)-1-oxoisoindolin-5-yl)-2,3-dihydrobenzofuran-3-yl)(methyl)carbamate (40 mg, 64% yield) as a yellow solid. LC-MS (ESI): mass calcd. For C₅₆H₆₉FN6010S, 1036.5; m/z found, 1037.5 [M+H]⁺.

Steps C-D: 2-amino-3-ethynyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)-N-methylquinoline-6-carboxamide

Example A287 was synthesized following the same procedures as described in Example A114 (4.6 mg, 2% yield over 4 steps) as a white solid. LC-MS (ESI): mass calcd. for C₆₃H₆₇FN₈O₉S, 1130.4/z found, 1131.2 [M+H]⁺. ¹H NMR (400 MHz, DMSO-d₆) δ 8.99 (s, 1H), 8.65 (s, 1H), 8.51 (t, J=5.9 Hz, 1H), 8.06 (s, 1H), 7.92-7.84 (m, 2H), 7.79-7.69 (m, 3H), 7.52 (d, J=7.8 Hz, 1H), 7.42 (d, J=7.8 Hz, 1H), 7.36-7.26 (m, 2H), 7.24 (s, 1H), 7.06 (s, 1H), 6.98 (d, J=7.8 Hz, 1H), 4.93 (s, 1H), 4.70-4.67 (m, 1H), 4.59 (d, J=9.2 Hz, 1H), 4.56-4.51 (m, 1H), 4.48 (s, 2H), 4.39-4.29 (m, 3H), 4.23-4.17 (m, 3H), 4.05-4.03 (m, 1H), 3.67-3.59 (m, 3H), 3.49-3.37 (m, 2H), 2.67 (s, 3H), 2.47 (s, 3H), 2.17-2.08 (m, 3H), 1.98-1.91 (m, 1H), 1.84-1.75 (m, 2H), 1.73-1.61 (m, 2H), 1.42-1.30 (m, 4H), 1.26-1.18 (m, 3H), 0.95 (s, 9H).

### Example A288. 2-amino-3-ethynyl-N-((S)-6-(6-((1′-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)-[1,4′-bipiperidin]-4-yl)carbamoyl) pyridin-3-yl)-2,3-dihydrobenzofuran-3-yl)-N-methylquinoline-6-carboxamide

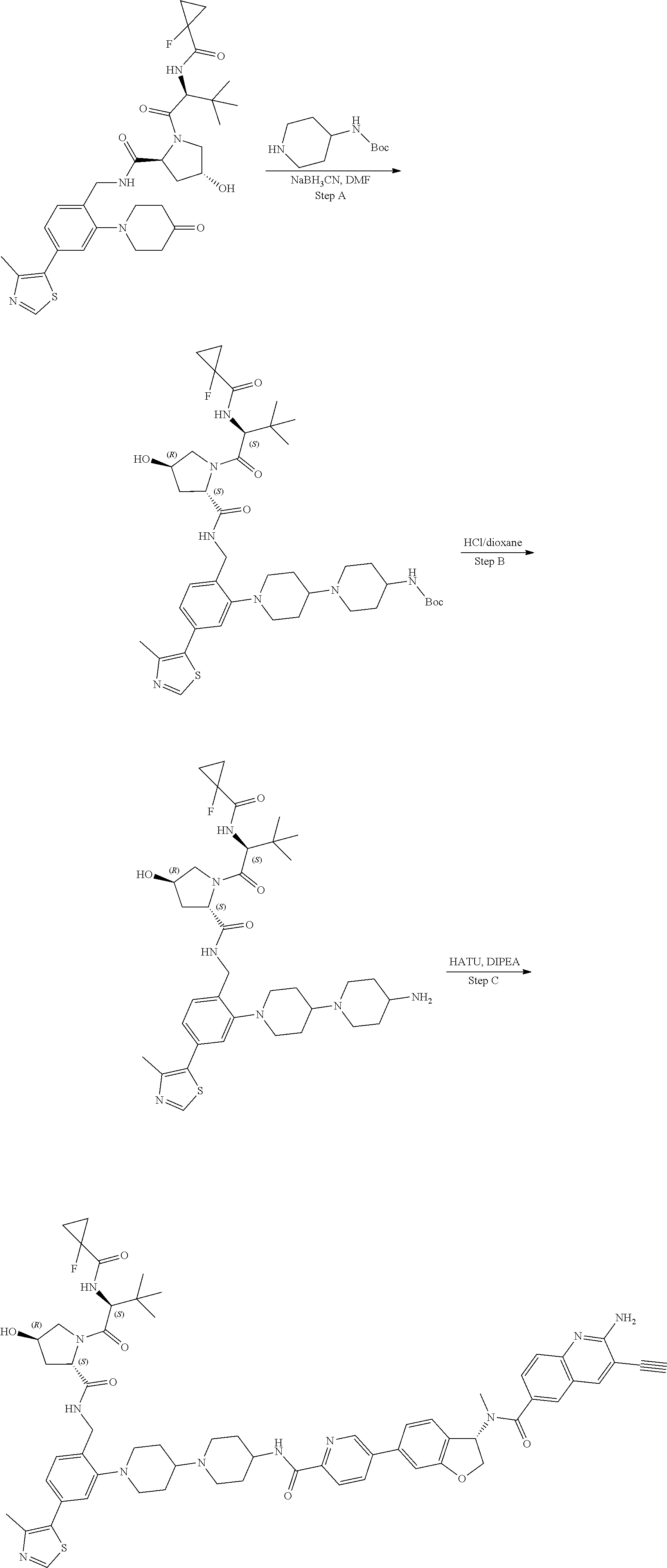

Example A288 was synthesized following the same procedures as described in Example 107 (3.1 mg, 4% yield over 3 steps) as a white solid. LC-MS (ESI): mass calcd. for C₆₃H₇₀FN₁₁O₇S, 1143.52/z found, 1144.8 [M+H]⁺. ¹H NMR (400 MHz, DMSO-d₆) δ 9.23 (s, 1H), 9.00 (s, 1H), 8.95 (d, J=1.9 Hz, 1H), 8.62-8.43 (m, 2H), 8.30 (dd, J=8.2, 2.2 Hz, 1H), 8.11 (d, J=8.2 Hz, 1H), 7.99 (s, 1H), 7.80 (d, J=8.4 Hz, 1H), 7.66 (d, J=8.4 Hz, 1H), 7.57 (d, J=8.0 Hz, 1H), 7.45 (d, J=8.4 Hz, 1H), 7.40 (d, J=7.0 Hz, 1H), 7.36 (s, 1H), 7.28 (dd, J=9.4, 2.7 Hz, 1H), 7.16 (d, J=6.6 Hz, 2H), 4.83 (s, 1H), 4.72-4.67 (m, 1H), 4.61 (d, J=9.4 Hz, 1H), 4.54-4.46 (m, 1H), 4.46-4.38 (m, 1H), 4.38-4.30 (m, 2H), 4.22-4.06 (m, 1H), 3.67-3.56 (m, 6H), 3.23-3.20 (m, 4H), 2.86-2.72 (m, 3H), 2.67 (s, 3H), 2.47 (s, 3H), 2.15-2.06 (m, 4H), 2.00-1.88 (m, 4H), 1.41-1.33 (m, 2H), 1.24-1.19 (m, 2H), 0.98 (s, 9H).

### Example A289. N-((S)-6-(6-(((18,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N,1-dimethyl-1H-pyrazolo[4,3-c]quinoline-8-carboxamide

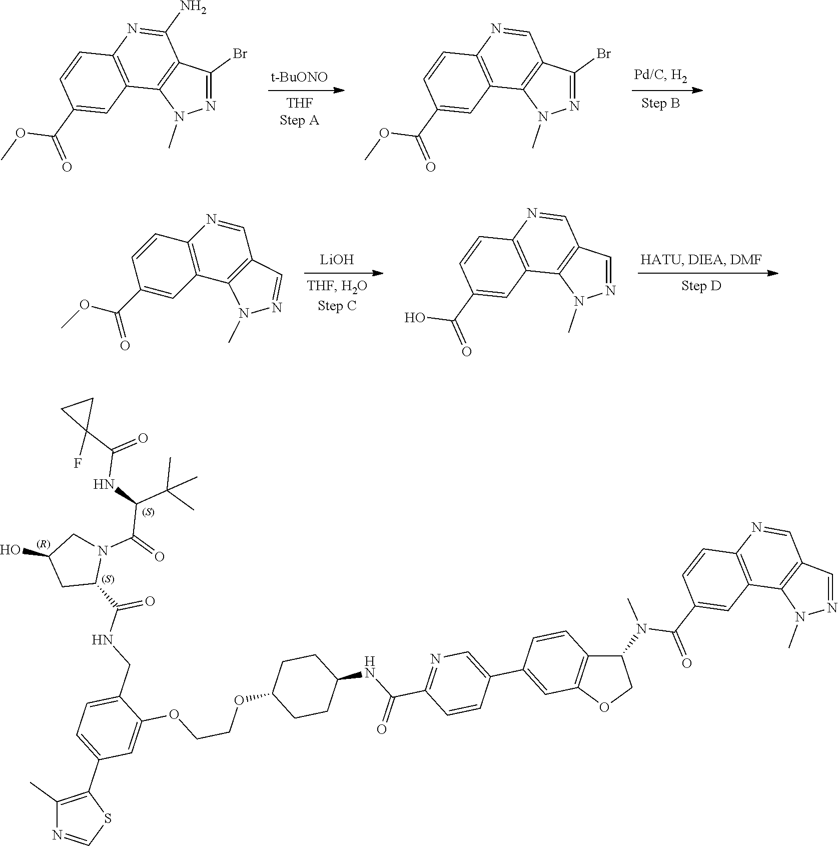

Step A: methyl 3-bromo-1-methyl-1H-pyrazolo[4,3-c]quinoline-8-carboxylate

To a solution of methyl methyl 4-amino-3-bromo-1-methyl-1H-pyrazolo[4,3-c]quinoline-8-carboxylate (130 mg, 389 μmol, 1.0 eq.) and tert-Butyl nitrite (2.3 g, 2.5 mL, 22.3 mmol, 57.4 eq.) in THF (9 mL) was stirred at 70° C. for 16 hours. The mixture was poured into water (30 mL) and extracted with EtOAc (30×3 mL). The combined organic layers were dried over anhydrous Na₂SO₄, filtered and the filtrate was concentrated to dryness. The residue was purified by column chromatography (silica gel, 0% EtOAc in PE~30% EtOAc in PE) to give methyl 3-cyclopropyl-4-hydroxy-1-methyl-1H-pyrazolo[4,3-c]quinoline-8-carboxylate (80 mg, 64% yield) as a yellow solid. LC-MS (ESI): mass calcd. For C₁₃H₁₀BrN₃O₂, 319.00; m/z found, 320 [M+H]⁺.

Step B: methyl 1-methyl-1H-pyrazolo[4,3-c]quinoline-8-carboxylate

To a solution of methyl 3-bromo-1-methyl-1H-pyrazolo[4,3-c]quinoline-8-carboxylate (55 mg, 171.8 μmol, 1.0 eq.) and PdCl₂ (dppf)-CH₂Cl₂ (14 mg, 17.18 μmol, 0.1 eq.) in DMF (3 mL) and BnOH (2 mL) was added TEA (86.92 mg, 859.0 μmol, 5.0 eq.). The reaction was stirred at 120° C. for 12 hours under CO. The reaction was purified by prep-HPLC (C18, MeCN/H2O, 0.1% FA, 0 to 55% gradient, 30 min run) to afford methyl 1-methyl-1H-pyrazolo[4,3-c]quinoline-8-carboxylate (15 mg, 36% yield) as a yellow solid. LC-MS (ESI): mass calcd. For: C13H1N302, 241.1; m/z found, 242 [M+H]⁺.

Steps C-D: N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N, 1-dimethyl-1H-pyrazolo[4,3-c]quinoline-8-carboxamide

Example A289 was synthesized following the same procedures as described in Example A29 (1.6 mg, 2% yield over 2 steps) as a white solid. LC-MS (ESI): mass calcd. for C₆₁H₆₇FN₁₀O₉S, 1134.5; m/z found, 1135.8 [M+H]⁺. ¹H NMR (400 MHz, MeOD-d₄) δ 9.25 (s, 1H), 8.87 (s, 2H), 8.72 (s, 1H), 8.35-8.24 (m, 2H), 8.21-8.11 (m, 2H), 7.96 (d, J=8.5 Hz, 1H), 7.66-7.52 (m, 1H), 7.47 (d, J=7.9 Hz, 1H), 7.39-7.31 (m, 1H), 7.29-7.18 (m, 1H), 7.07-7.00 (m, 2H), 4.81-4.76 (m, 2H), 4.73 (s, 1H), 4.64 (d, J=8.4 Hz, 1H), 4.60 (s, 3H), 4.57 (s, 3H), 4.53-4.40 (m, 3H), 4.25-4.21 (m, 2H), 3.95-3.89 (m, 3H), 3.87-3.77 (m, 2H), 2.90-2.78 (m, 2H), 2.49 (s, 3H), 2.27-2.20 (m, 1H), 2.20-2.15 (m, 2H), 2.11 (s, 1H), 2.08-2.02 (m, 2H), 1.56-1.43 (m, 4H), 1.32-1.28 (m, 4H), 1.02 (s, 9H).

### Example A290. 4-amino-N-((S)-6-(6-(9-(2-(2-(2-(2-(((2S,4S)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)-3,9-diazaspiro[5.5]undecane-3-carbonyl)pyridin-3-yl)-2,3-dihydrobenzofuran-3-yl)-N,1-dimethyl-1H-pyrazolo[4,3-c]quinoline-8-carboxamide

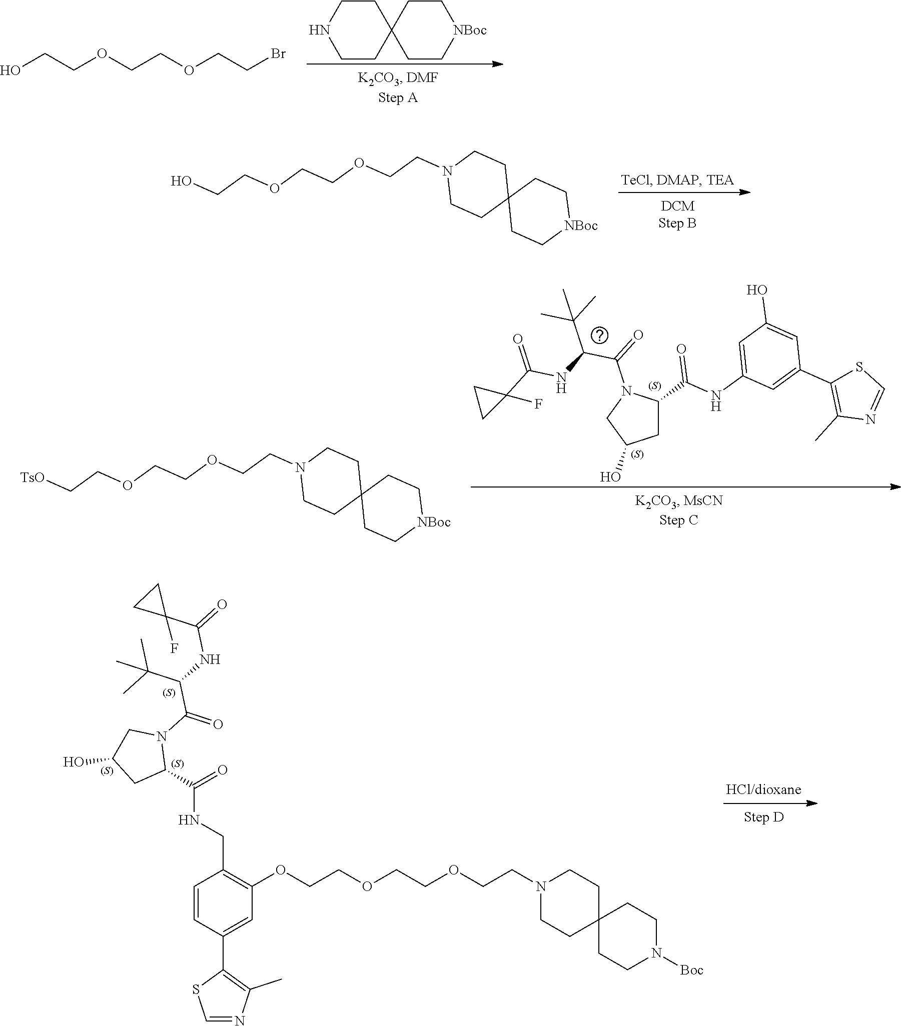

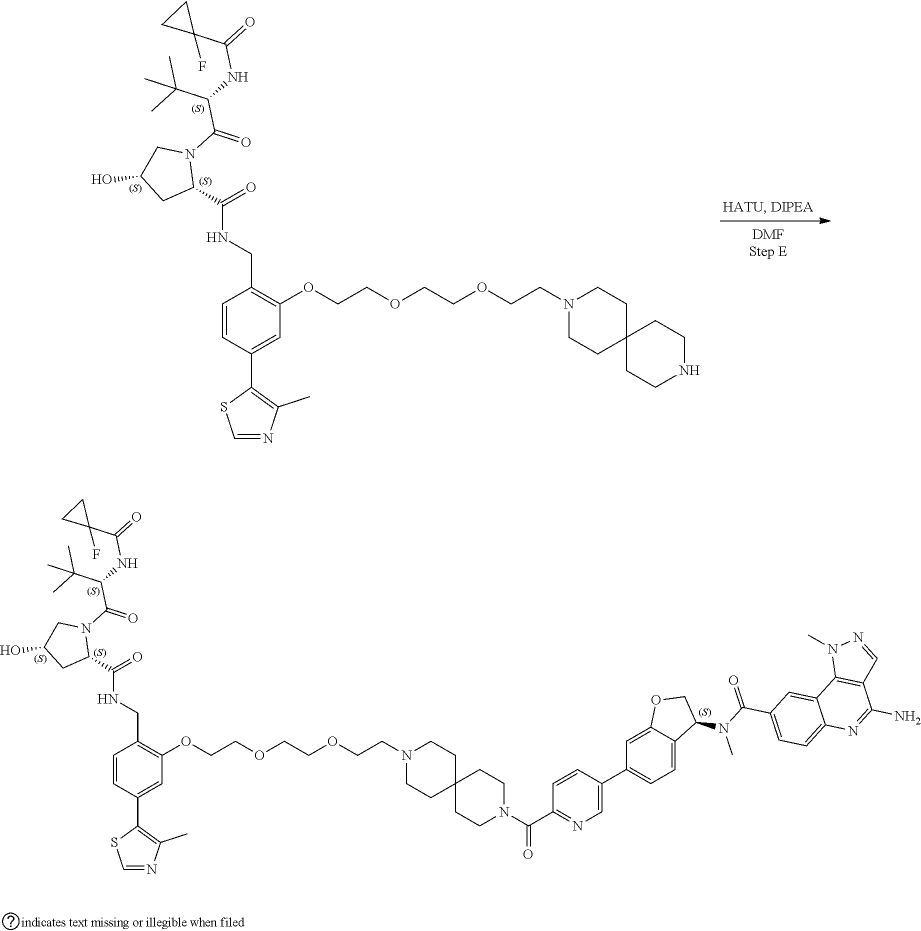

Step A: tert-butyl 9-(2-(2-(2-hydroxyethoxy)ethoxy)ethyl)-3,9-diazaspiro[5.5]undecane-3-carboxylate

To a solution of 2-(2-(2-bromoethoxy)ethoxy) ethan-1-ol (544 mg, 2.56 mmol, 1.3 eq.) in MeCN (10 mL) was added tert-butyl 3,9-diazaspiro[5.5]undecane-3-carboxylate (500 mg, 1.97 mmol, 1.0 eq.) and K₂CO₃ (815 mg, 5.9 mmol, 3.0 eq.) at room temperature. The mixture was then stirred at 80° C. for 16 hours under N₂ atmosphere. The resulting solution was diluted with 60 mL of water and extracted with EtOAc (50 mL×3). The combined organic layers were washed with brine (100 mL), dried over Na₂SO₄ and concentrated under reduced pressure. The resulting solution was concentrated under reduced pressure. The residue was purified by column chromatography (silica gel, 0% MeOH in DCM~4% MeOH in DCM) to afford tert-butyl 9-(2-(2-(2-hydroxyethoxy)ethoxy)ethyl)-3,9-diazaspiro[5.5]undecane-3-carboxylate (750 mg, 98% yield) as a yellow oil. LC-MS (ESI): mass calcd. For C₂₀H₃₈N₂O₅, 386.3; m/z found, 387.6 [M+H]⁺.

Step B: tert-butyl 9-(2-(2-(2-(tosyloxy)ethoxy)ethoxy)ethyl)-3,9-diazaspiro[5.5[undecane-3-carboxylate

To solution of tert-butyl 9-(2-(2-(2-hydroxyethoxy)ethoxy)ethyl)-3,9-diazaspiro[5.5]undecane-3-carboxylate (250 mg, 647 μmol, 1.0 eq.) in DCM (5 mL) was added TEA (131 mg, 1.29 mmol, 2.0 eq.), DMAP (16 mg, 129 μmol, 0.2 eq.) and p-Toluenesulfonyl chloride (247 mg, 1.29 mmol, 2.0 eq.) at room temperature. The mixture was then stirred at room temperature for 16 hours under N₂ atmosphere. The resulting solution was concentrated under reduced pressure. The residue was purified by column chromatography (silica gel, 0% MeOH in DCM~4% MeOH in DCM) to afford tert-butyl 9-(2-(2-(2-(tosyloxy)ethoxy)ethoxy)ethyl)-3,9-diazaspiro[5.5]undecane-3-carboxylate (200 mg, 57% yield) as a yellow oil. LC-MS (ESI): mass calcd. For C27H44N207S, 540.3; m/z found, 541.3 [M+H]⁺

Steps C-E:-amino-N-((S)-6-(6-(9-(2-(2-(2-(2-(((2S,4S)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)ethoxy)ethyl)-3,9-diazaspiro[5.5]undecane-3-carbonyl)pyridin-3-yl)-2,3-dihydrobenzofuran-3-yl)-N,1-dimethyl-1H-pyrazolo[4,3-c]quinoline-8-carboxamide

Example A290 was synthesized following the same procedures as described in Example A29 (4.5 mg, 5% yield over 3 steps) as a white solid. LC-MS (ESI): mass calcd. for C68H81FN₁₂O₁₀S, 1276.6/z found, 1277.9 [M+H]⁺. ¹H NMR (400 MHz, DMSO-d₆) δ 8.98 (s, 1H), 8.88 (d, J=1.8 Hz, 1H), 8.62-8.50 (m, 1H), 8.34 (s, 1H), 8.26 (s, 1H), 8.22-8.17 (m, 1H), 7.68-7.62 (m, 2H), 7.61-7.54 (m, 2H), 7.42-7.36 (m, 2H), 7.33-7.28 (m, 2H), 7.16 (s, 2H), 7.04 (s, 1H), 6.97-6.92 (m, 1H), 5.44 (s, 1H), 4.77-4.73 (m, 1H), 4.54 (d, J=8.8 Hz, 1H), 4.48-4.44 (m, 1H), 4.43 (s, 3H), 4.38-4.30 (m, 1H), 4.27-4.20 (m, 2H), 4.20-4.16 (m, 2H), 3.89-3.82 (m, 1H), 3.82-3.77 (m, 2H), 3.64-3.58 (m, 4H), 3.56-3.49 (m, 4H), 3.47-3.43 (m, 1H), 3.36-3.33 (m, 7H), 2.72 (s, 3H), 2.45 (s, 3H), 2.38-2.34 (m, 1H), 1.91 (s, 1H), 1.79-1.72 (m, 1H), 1.53-1.41 (m, 6H), 1.39-1.30 (m, 4H), 1.24-1.18 (m, 2H), 0.96 (s, 9H).

### Example A291. 2-amino-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-3-(1-methylcyclopropyl)quinoline-6-carboxamide

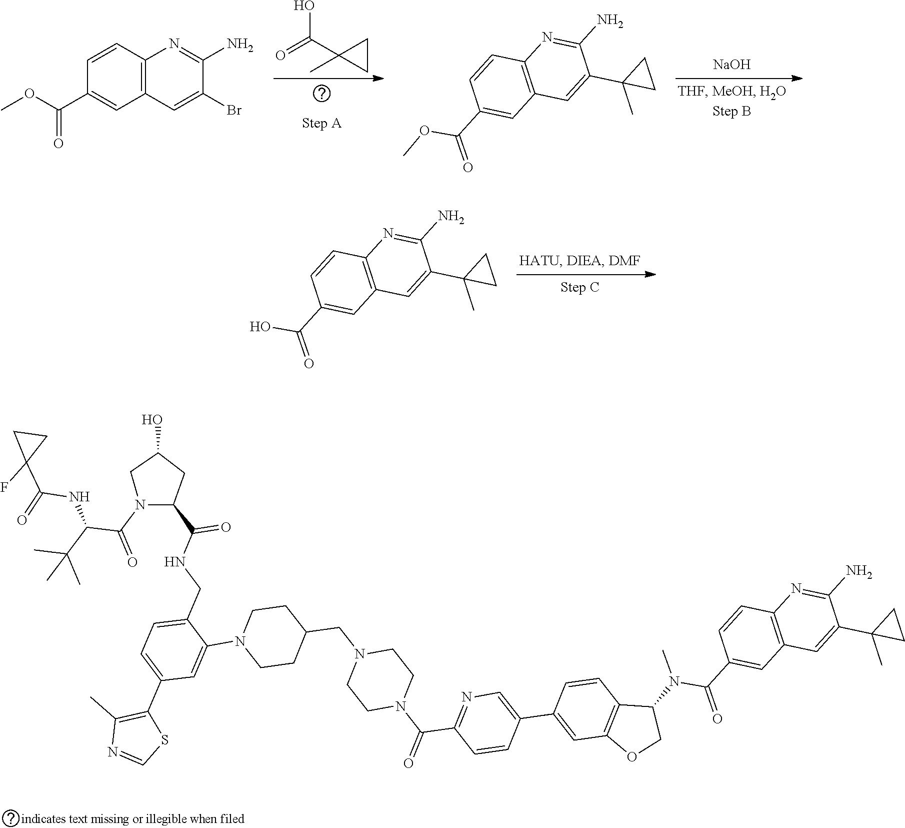

Step A: methyl 2-amino-3-(1-methylcyclopropyl)quinoline-6-carboxylate

To a stirred mixture of methyl 2-amino-3-bromoquinoline-6-carboxylate (600 mg, 2.13 mmol, 1.0 eq.), 1-methylcyclopropane-1-carboxylic acid (427 mg, 4.27 mmol, 2.0 eq.), isoindoline-1,3-dione (314 mg, 2.13 mmol, 1.0 eq.), and (4,4′-Di-t-butyl-2,2′-bipyridine) bis [3,5-difluoro-2-(5-trifluoromethyl-2-pyridinyl-kN) phenyl-kC]iridium (III) hexafluorophosphate (239 mg, 213 μmol, 1.0 eq.) in DMA (8 mL) was added Cs₂CO₃ (1.39 g, 4.27 mmol, 2.0 eq.). The resulting mixture was stirred at room temperature for 12 hours under 450 nm lamp. The reaction mixture was quenched with water (20 mL), and then extracted with EtOAc (20 mL×2). The combined organic layers were washed with brine, dried over anhydrous Na₂SO₄ and concentrated. The residue was purified by column chromatography (silica gel, 0% EtOAc in PE~20% EtOAc in PE) to afford methyl 2-amino-3-(1-methylcyclopropyl)quinoline-6-carboxylate (40 mg, 7% yield) as a white solid. LC-MS (ESI): mass calcd. For C₁₅H₁₅FN₂O₂, 256.12; m/z found, 257.3 [M+H]⁺.

Steps B-C: 2-amino-N-(S)-6-(6-14-(1-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methyl-3-(1-methylcyclopropyl)quinoline-6-carboxamide

Example A291 was synthesized following the same procedures as described in Example A114 (2.2 mg, 3% yield over 2 steps) as a white solid. LC-MS (ESI): mass calcd. for C₆₅H₇₆FN₁₁O₇S, 1173.56; m/z found, 1175.0 [M+H]⁺. ¹HNMR (400 MHz, DMSO-d₆) δ 8.97 (s, 1H), 8.91 (s, 1H), 8.49 (t, J=5.8 Hz, 1H), 8.25-8.17 (m, 2H), 7.65 (d, J=7.9 Hz, 2H), 7.56-7.51 (m, 2H), 7.41 (t, J=8.6 Hz, 2H), 7.33 (s, 1H), 7.28 (dd, J=9.8, 1.9 Hz, 1H), 7.10 (d, J=8.9 Hz, 2H), 6.81 (s, 1H), 5.17 (d, J=3.5 Hz, 1H), 4.76-4.66 (m, 2H), 4.60 (d, J=9.0 Hz, 1H), 4.48 (t, J=8.1 Hz, 1H), 4.43-4.22 (m, 4H), 3.70-3.55 (m, 7H), 3.09-3.02 (m, 3H), 2.74-2.60 (m, 9H), 2.45 (s, 3H), 2.11-2.06 (m, 1H), 1.94-1.90 (m, 1H), 1.83 (d, J=11.1 Hz, 2H), 1.43 (s, 3H), 1.40-1.31 (m, 5H), 1.21 (dd, J=8.3, 2.7 Hz, 2H), 0.97 (s, 9H), 0.94 (d, J=4.1 Hz, 2H), 0.83 (d, J=4.9 Hz, 2H).

### Example A292. N6-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N6-methyl-2-(methylamino) quinoline-3,6-dicarboxamide

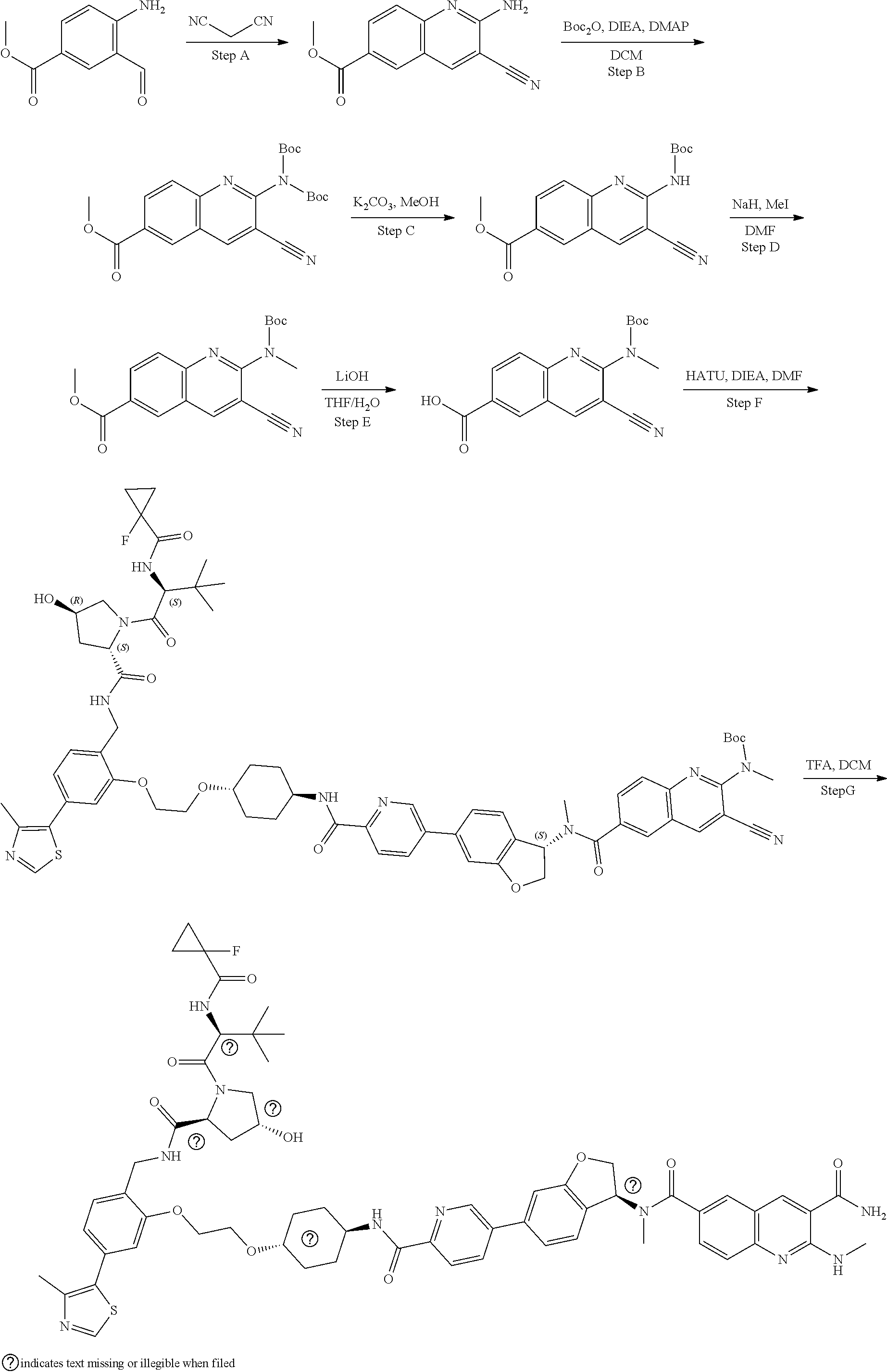

Steps A-F: tert-butyl (3-cyano-6-(((S)-6-(6-(((1S,4S)-4-(2-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)(methyl)carbamoyl) quinolin-2-yl)(methyl)carbamate

Tert-butyl (3-cyano-6-(((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)(methyl)carbamoyl) quinolin-2-yl)(methyl)carbamate was synthesized following the same procedures as described in Example A199 and A249.

Step G: N6-(S)-6-(6-(((15,4S)-4-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N6-methyl-2-(methylamino) quinoline-3,6-dicarboxamide

To the solution of tert-butyl (3-cyano-6-(((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)(methyl)carbamoyl) quinolin-2-yl)(methyl)carbamate (8.00 mg, 6.48 μmol, 1 eq) in DCM (3 mL) was added TFA (1 mL) at room temperature. The mixture was stirred at room temperature for 1 hour under N₂ atmosphere. The mixture was purified by Prep-HPLC (C18, 20~95%, MeCN in H₂O with 0.1% HCOOH) to afford N6-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-2-ethoxy)cyclohexyl)carbamoyl)pyridin-3-yl)-2,3-dihydrobenzofuran-3-yl)-N6-methyl (methylamino) quinoline-3,6-dicarboxamide (2.5 mg, 33% yield) as a white solid. LC-MS (ESI): mass calcd, for C₆₁H₆₉FN₁₀O₁₀S, 1152.49; m/z found, 1153.9 [M+H]⁺. ¹H NMR (400 MHz, DMSO-d₆) δ 9.85 (s, 1H), 9.08-8.88 (m, 2H), 8.81 (s, 1H), 8.63-8.43 (m, 3H), 8.27 (s, 1H), 8.13-7.91 (m, 5H), 7.58 (s, 1H), 7.46-7.26 (m, 4H), 7.06 (s, 1H), 6.98 (s, 1H), 4.97-4.68 (m, 2H), 4.59 (s, 1H), 4.53 (s, 1H), 4.39-4.24 (m, 4H), 4.18 (s, 3H), 3.37 (s, 4H), 3.18 (s, 4H), 2.68 (s, 3H), 2.47 (s, 3H), 2.13-2.01 (m, 3H), 1.98-1.80 (m, 3H), 1.58-1.44 (m, 2H), 1.42-1.28 (m, 4H), 1.25-1.15 (m, 2H), 0.96 (s, 9H).

### Example A293. 3-cyano-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-2-(methylamino) quinoline-6-carboxamide

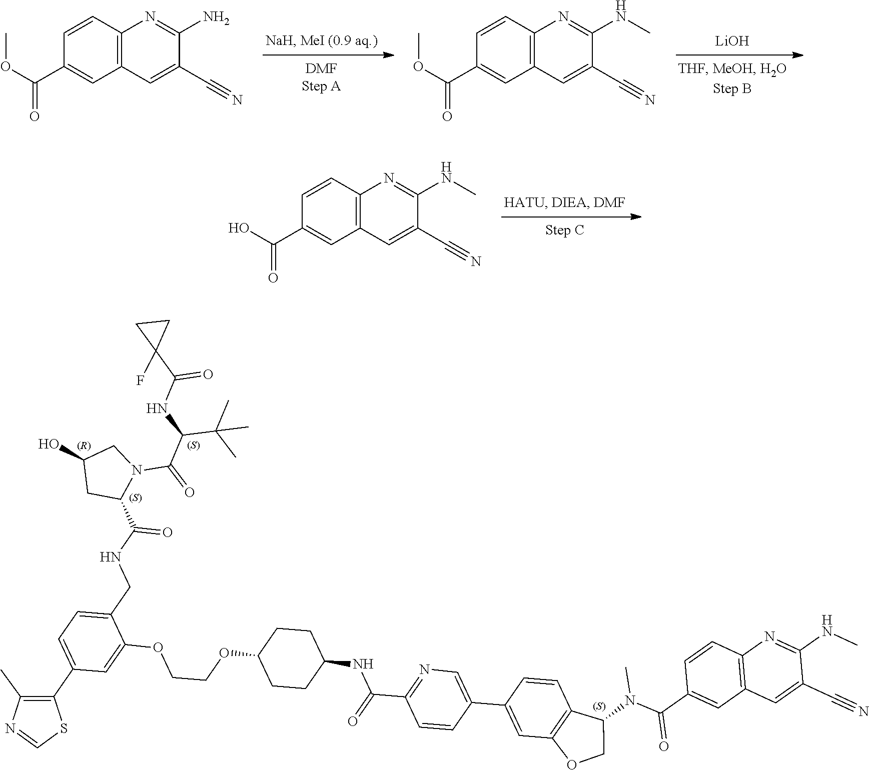

Example A293 was synthesized following the same procedures as described in Example A249 (3 mg, 8% yield over 3 steps) as a white solid. LC-MS (ESI): mass calcd. for C₆₁H₆₇FN₁₀O₉S, 1134.48/z found, 1135.7 [M+H]⁺. ¹H NMR (400 MHz, DMSO-d₆) δ 8.99 (s, 1H), 8.92 (s, 1H), 8.75 (s, 1H), 8.49 (d, J=6.6 Hz, 2H), 8.28 (dd, J=8.1, 2.1 Hz, 1H), 8.08 (d, J=8.2 Hz, 1H), 7.95 (s, 1H), 7.79 (dd, J=8.7, 1.9 Hz, 1H), 7.64 (d, J=9.2 Hz, 1H), 7.56 (d, J=7.8 Hz, 1H), 7.46-7.37 (m, 3H), 7.34 (s, 1H), 7.31-7.27 (m, 1H), 7.05 (s, 1H), 6.97 (d, J=8.0 Hz, 1H), 4.85-4.66 (m, 2H), 4.60 (d, J=9.2 Hz, 1H), 4.52 (t, J=8.1 Hz, 1H), 4.38-4.34 (m, 1H), 4.32-4.16 (m, 4H), 3.80 (d, J=4.1 Hz, 3H), 3.67-3.60 (m, 3H), 3.30-3.23 (m, 1H), 2.96 (d, J=4.2 Hz, 3H), 2.67 (s, 3H), 2.47 (s, 3H), 2.10-2.02 (m, 3H), 1.94-1.82 (m, 3H), 1.56-1.47 (m, 2H), 1.41-1.29 (m, 4H), 1.26-1.17 (m, 2H), 0.96 (s, 9H)

### Example A294. 2-amino-3-ethynyl-N-((3S)-6-(6-(5-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl) octahydropyrrolo[3,4-c]pyrrole-2-carbonyl)pyridin-3-yl)-2,3-dihydrobenzofuran-3-yl)-N-methylquinoline-6-carboxamide

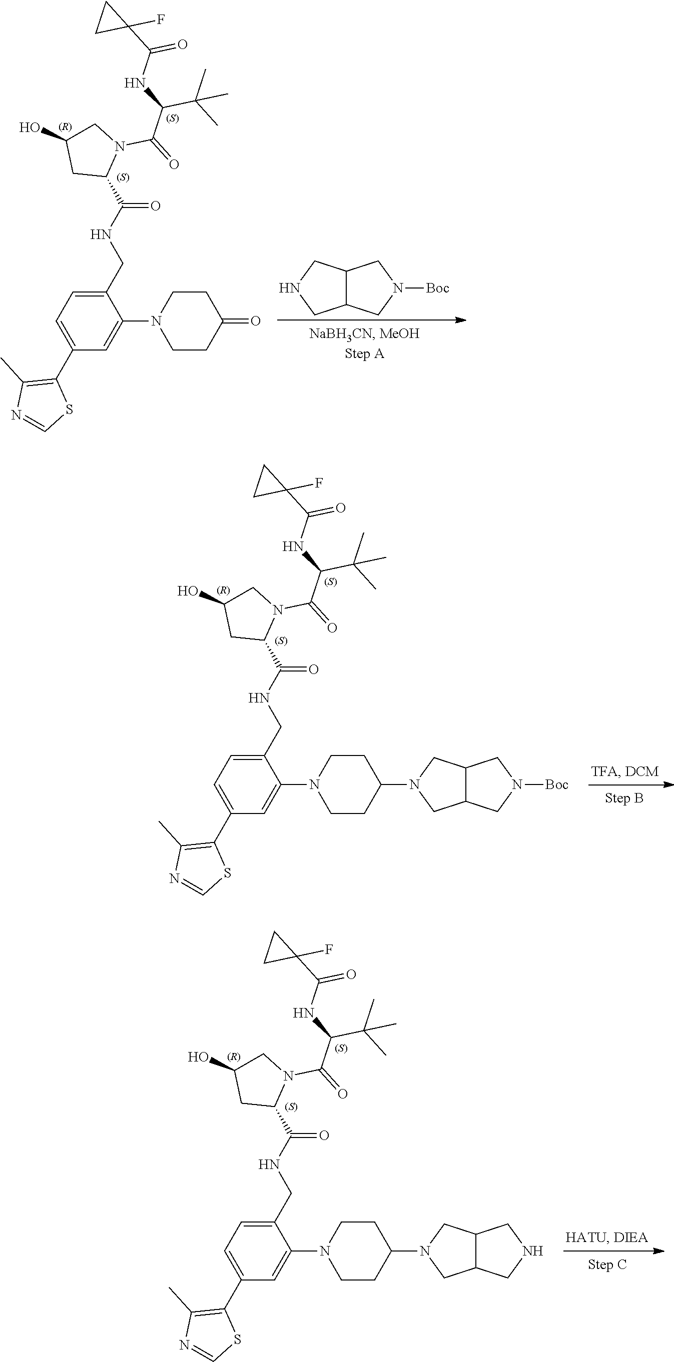

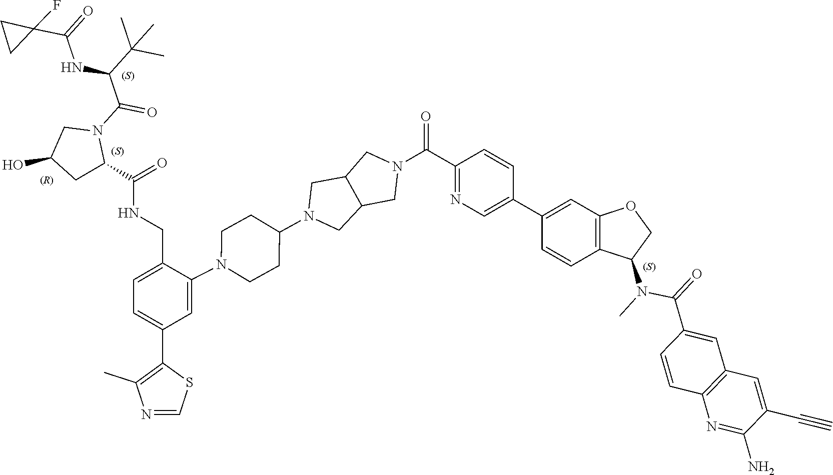

Example A294 was synthesized following the same procedures as described in Example A107 (2.0 mg, 7% yield over 3 steps) as a white solid. LC-MS (ESI): mass calcd. for C₆₄H₇₀FN₁₁O₇S, 1155.52; m/z found, 1156.5 [M+H]⁺. H NMR (400 MHz, DMSO-d₆) δ 8.97 (s, 1H), 8.91 (s, 1H), 8.49 (t, J=5.7 Hz, 1H), 8.29 (s, 1H), 8.19 (d, J=8.2 Hz, 1H), 8.15 (s, 1H), 7.88 (s, 1H), 7.77 (d, J=8.2 Hz, 1H), 7.66 (dd, J=8.7, 1.9 Hz, 1H), 7.53 (t, J=7.4 Hz, 2H), 7.43 (d, J=7.7 Hz, 1H), 7.38 (d, J=7.7 Hz, 1H), 7.32 (s, 1H), 7.28 (d, J=8.5 Hz, 1H), 7.10 (d, J=7.7 Hz, 2H), 6.72 (s, 2H), 5.17 (d, J=3.1 Hz, 1H), 4.76 (s, 1H), 4.69-4.66 (m, 1H), 4.63-4.57 (m, 2H), 4.48 (t, J=8.3 Hz, 1H), 4.43-4.33 (m, 2H), 4.32-4.25 (m, 1H), 3.92-3.82 (m, 2H), 3.68-3.64 (m, 1H), 3.61 (s, 1H), 3.58 (s, 1H), 3.56-3.48 (m, 3H), 3.09-3.01 (m, 2H), 2.86-2.80 (m, 2H), 2.74 (d, J=14.0 Hz, 2H), 2.68 (s, 3H), 2.66-2.60 (m, 1H), 2.58 (s, 1H), 2.45 (s, 3H), 2.23-2.13 (m, 1H), 2.10-2.04 (m, 1H), 1.97-1.85 (m, 3H), 1.65-1.53 (m, 2H), 1.42-1.31 (m, 2H), 1.24-1.18 (m, 2H), 0.96 (s, 9H).

### Example A295. 2-amino-3-ethynyl-N-((3S)-6-(6-(8-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)-3,8-diazabicyclo[3.2.1]octane-3-carbonyl)pyridin-3-yl)-2,3-dihydrobenzofuran-3-yl)-N-methylquinoline-6-carboxamide

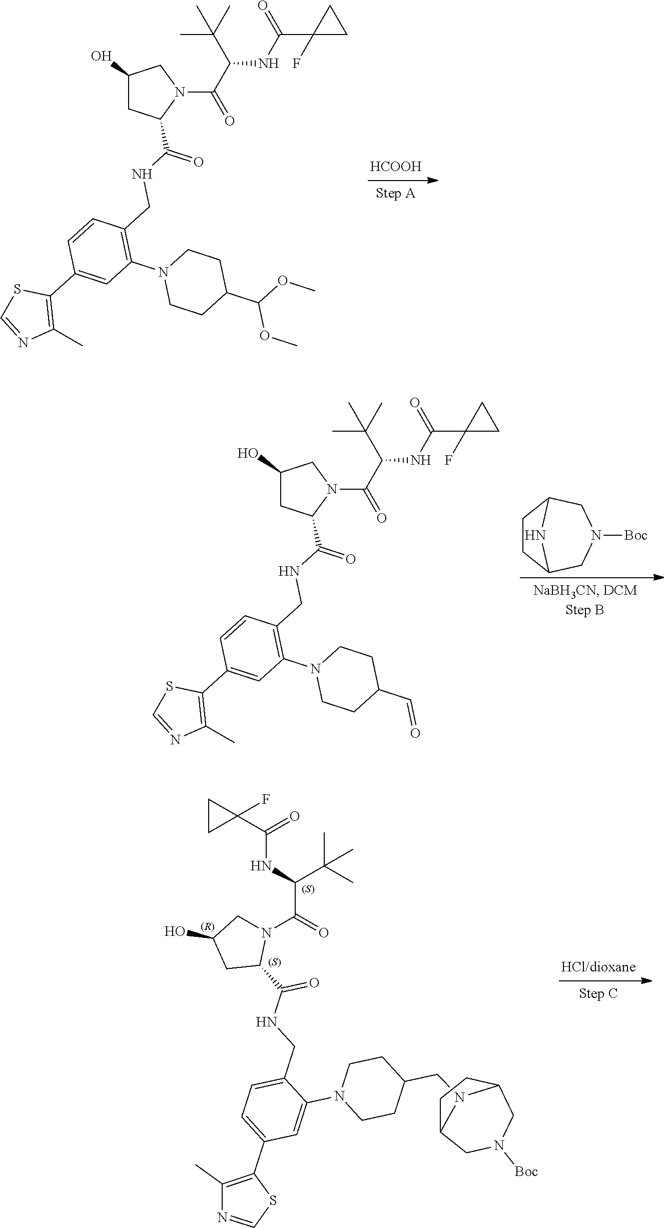

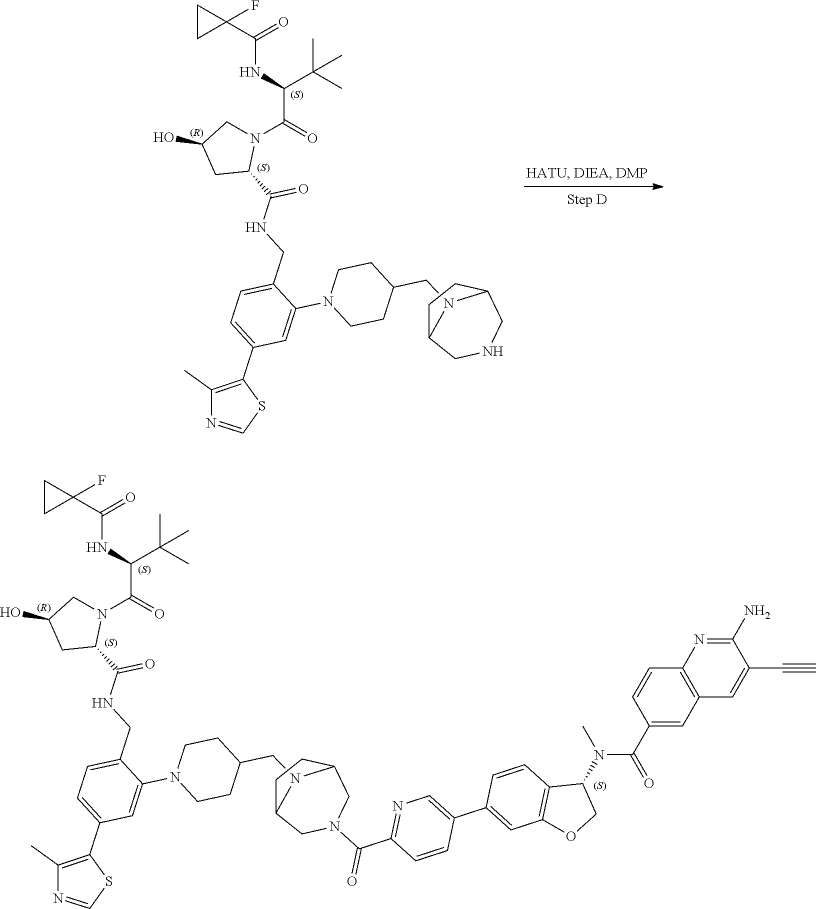

Example A295 was synthesized following the same procedures as described in Example A107 (11.3 mg, 18% yield over 4 steps) as a white solid. LC-MS (ESI): mass calcd. for C₆₅H₇₂FN₁₁O₇S, 1169.53 m/z found, 1170.5 [M+H]⁺. ¹H NMR (400 MHz, DMSO-d₆) δ 9.76 (s, 1H), 8.99 (s, 1H), 8.95 (s, 1H), 8.69-8.49 (m, 3H), 8.31-8.25 (m, 1H), 8.07 (s, 1H), 7.90 (d, J=8.3 Hz, 1H), 7.80 (d, J=8.2 Hz, 1H), 7.76-7.66 (m, 1H), 7.56 (d, J=7.8 Hz, 1H), 7.44 (d, J=8.3 Hz, 1H), 7.40 (d, J=7.6 Hz, 1H), 7.35 (s, 1H), 7.27 (d, J=6.9 Hz, 1H), 7.16-7.11 (m, 2H), 4.94 (s, 1H), 4.85-4.74 (m, 1H), 4.69 (d, J=6.1 Hz, 2H), 4.61 (d, J=9.2 Hz, 2H), 4.52-4.48 (m, 2H), 4.40-4.33 (m, 4H), 4.23 (s, 1H), 4.05-3.99 (m, 2H), 3.78 (d, J=13.7 Hz, 1H), 3.68-3.59 (m, 2H), 3.50-3.45 (m, 1H), 3.16-3.09 (m, 2H), 3.03 (s, 2H), 2.77-2.65 (m, 5H), 2.47 (s, 3H), 2.30-2.16 (m, 2H), 2.15-2.05 (m, 2H), 1.99-1.83 (m, 5H), 1.54-1.44 (m, 2H), 1.41-1.32 (m, 2H), 1.25-1.22 (m, 2H), 0.97 (s, 9H).

### Example A297. 3-ethynyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-2-(methylamino) quinoline-6-carboxamide

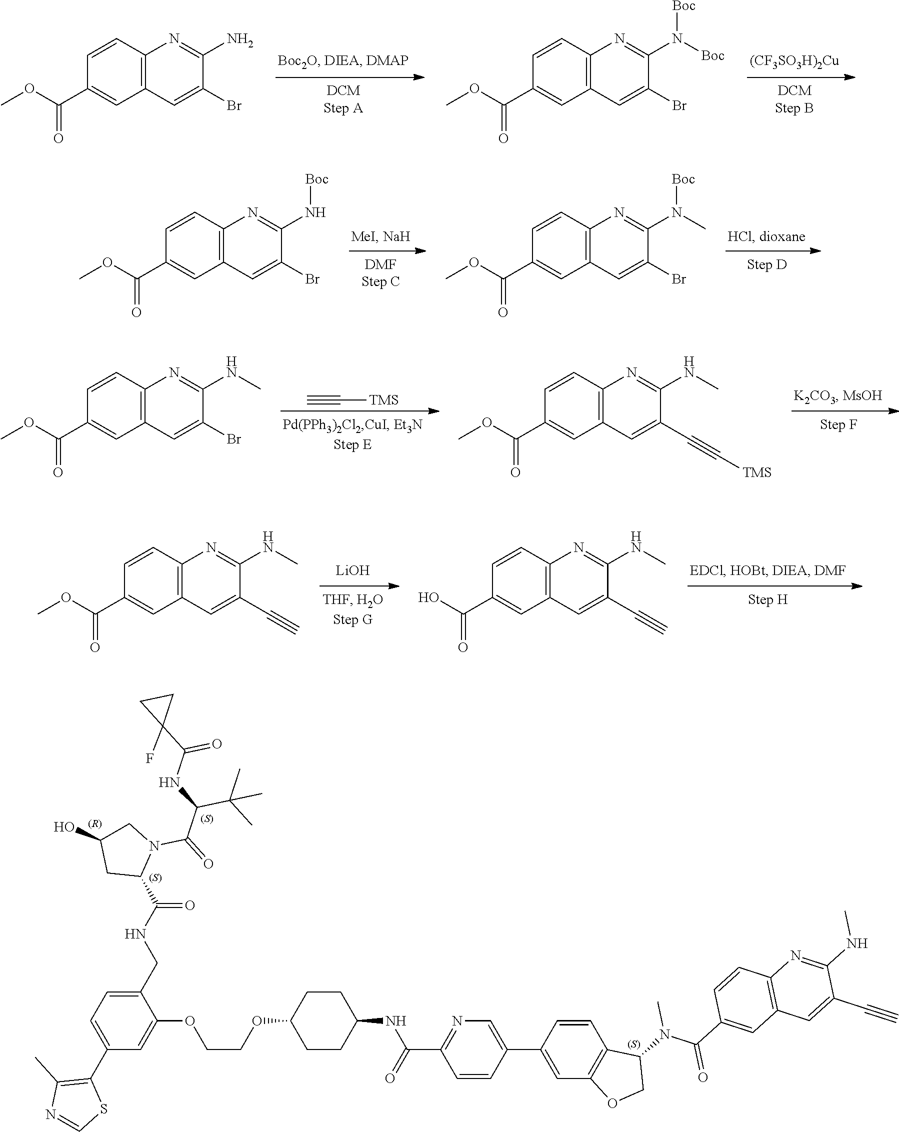

Step A: methyl 2-(bis(tert-butoxycarbonyl)amino)-3-bromoquinoline-6-carboxylate

To a stirred mixture of methyl 2-amino-3-bromoquinoline-6-carboxylate (500 mg, 1.78 mmol, 1.0 eq.) in DCM (10 mL) were added Boc₂O (800 mg, 1.78 mmol, 2.0 eq.), TEA (540 mg, 744 μL, 5.34 mmol, 3.0 eq.). DMAP (43.5 mg, 356 μmol, 0.2 eq.) was then added and the mixture was stirred at room temperature for 16 hours. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash silica chromatography, eluting with a gradient of 0~3% MeOH in DCM to afford methyl 2-(bis(tert-butoxycarbonyl)amino)-3-bromoquinoline-6-carboxylate (160 mg, 19% yield) as white solid. LC-MS (ESI): mass calcd. For C₂₁H₂₅BrN₂O₆, 480.09; m/z found, 481.3 [M+H]⁺.

Step B: methyl 3-bromo-2-((tert-butoxycarbonyl)amino) quinoline-6-carboxylate

A solution of methyl 2-(bis(tert-butoxycarbonyl)amino)-3-bromoquinoline-6-carboxylate (400 mg, 831 μmol, 1.0 eq.), Copper (II) trifluoromethanesulfonate (300 mg, 831 μmol, 1.0 eq.) in DCM (8 mL) with stirring at room temperature. The mixture was stirred at room temperature for 2 hours. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash silica gel chromatography, elution with a gradient of 0 to 10% MeOH in DCM. The desired fractions were evaporated to dryness to afford methyl 3-bromo-2-((tert-butoxycarbonyl)amino) quinoline-6-carboxylate (300 mg, 95% yield) as a solid. LC-MS (ESI): mass calcd. For C₁₆H₁₇BrN₂O₄, 380.04; m/z found, 381.0 [M+H]⁺

Step C: methyl 3-bromo-2-((tert-butoxycarbonyl)(methyl)amino) quinoline-6-carboxylate

To a suspension of methyl 3-bromo-2-((tert-butoxycarbonyl)amino) quinoline-6-carboxylate (200 mg, 525 μmol, 1.0 eq.) in anhydrous DMF (8 mL) was added NaH (25 mg, 1.05 mmol, 2.0 eq.) under nitrogen at 0° C. The white suspension was stirred at 0° C. for 1 hour, then Methyl iodide (82 mg, 37.3 μL, 577 μmol, 1.1 eq.) was slowly added. The resulting solution was stirred at 0° C. and allowed to warm to room temperature. After 3 hours, the reaction was quenched with brine (20 mL), and the mixture was extracted with EtOAc (20 mL×3). The organic extract was dried over anhydrous Na₂SO₄, filtered and evaporated. The crude product was purified by flash silica chromatography, eluting with a gradient of 0~100% EtOAc in PE to afford methyl 3-bromo-2-((tert-butoxycarbonyl)(methyl)amino) quinoline-6-carboxylate (150 mg, 72% yield) as a solid. LC-MS (ESI): mass calcd. For C₁₇H₁₉BrN₂O₄, 394.05; m/z found, 394.9 [M+H]⁺.

Step D: methyl 3-bromo-2-(methylamino) quinoline-6-carboxylate

To a solution of methyl 3-bromo-2-((tert-butoxycarbonyl)(methyl)amino) quinoline-6-carboxylate (150 mg, 380 μmol, 1.0 eq.) in DCM (1 mL) was added hydrochloric acid (1 mL, 4 M/L), the reaction mixture was stirred at room temperature for 1 hour. The reaction mixture was concentrated under reduced pressure to afford methyl 3-bromo-2-(methylamino) quinoline-6-carboxylate (120 mg, 89% yield) as a white solid. LC-MS (ESI): mass calcd. For C₁₂H₁₁BrN₂O₂, 294.00; m/z found, 295.1 [M+H]⁺.

Step E: methyl 2-(methylamino)-3-((trimethylsilyl) ethynyl)quinoline-6-carboxylate

To a solution of methyl 3-bromo-2-(methylamino) quinoline-6-carboxylate (80 mg, 271 μmol, 1.0 eq.) in DMF (5 mL) was added CuI (5 mg, 27.1 μmol, 0.1 eq.), Phosphine, tricyclohexyl-(15 mg, 54.2 μmol, 0.2 eq.), Bis-(triphenylphosphino)-palladous chloride (19 mg, 27.1 μmol, 0.1 eq.), TEA (274 mg, 378 μL, 2.71 mmol, 10.0 eq.) and ethynyltrimethylsilane (160 mg, 1.63 mmol, 6.0 eq.) under N₂ atmosphere. The mixture was stirred at 90° C. for 16 hours. The mixture was quenched water (20 mL), extracted with EtOAc (20 mL×3) and the layers were separated. The organic layer was washed with brine (10 mL×3), dried over Na₂SO₄, filtered and concentrated. The product was purified by flash chromatography (100-200 mesh silica gel, 0%-100% EA in PE) to afford methyl 2-(methylamino)-3-((trimethylsilyl) ethynyl)quinoline-6-carboxylate (60 mg, 71% yield) as a yellow solid. LC-MS (ESI): mass calcd. For C₁₇H₂₀N₂O₂Si, 312.13; m/z found, 313.1 [M+H]⁺.

Step F: methyl 3-ethynyl-2-(methylamino) quinoline-6-carboxylate

A solution of methyl 2-(methylamino)-3-((trimethylsilyl)etbynyl)quinoline-6-carboxylate (40.0 mg, 128 μmol, 1.0 eq.), K₂CO₃ (53 mg, 384 μmol, 3.0 eq.) in MeOH (3 mL) with stirring at room temperature. The mixture was stirred at room temperature for 3 hours. The mixture was quenched water (20 mL), extracted with EtOAc (20 mL×3) and the layers were separated. The organic layer was washed with brine (10 mL×3), dried over Na₂SO₄, filtered and concentrated. The crude product was purified by flash silica chromatography, eluting with a gradient of 0~3% MeOH in DCM to afford methyl 3-ethynyl-2-(methylamino) quinoline-6-carboxylate (25 mg, 81% yield) as a solid. LC-MS (ESI): mass calcd. For C₁₄H₁₂N₂O₂, 240.09; m/z found, 241.1 [M+H]⁺.

Steps G-H: 3-ethynyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methyl-2-(methylamino) quinoline-6-carboxamide

Example A297 was synthesized following the same procedures as described in Example A114 (2.4 mg, 7% yield over 2 steps) as a white solid. LC-MS (ESI): mass calcd. for C₆₂H₆₈FN₉O₉S, 1133.48; m/z found, 1134.9 [M+H]⁺. ¹H NMR (400 MHz, DMSO-d₆) δ 8.99 (s, 1H), 8.92 (s, 1H), 8.52-8.45 (m, 3H), 8.29-8.26 (m, 1H), 8.08 (d, J=8.2 Hz, 1H), 7.99 (s, 1H), 7.87-7.80 (m, 2H), 7.56 (d, J=7.7 Hz, 1H), 7.43-7.38 (m, 2H), 7.35 (s, 1H), 7.32-7.27 (m, 1H), 7.07-7.04 (m, 1H), 6.99-6.96 (m, 1H), 4.91 (s, 1H), 4.82-4.67 (m, 2H), 4.60 (d, J=9.1 Hz, 1H), 4.55-4.50 (m, 1H), 4.37-4.15 (m, 6H), 3.83-3.78 (m, 4H), 3.68-3.65 (m, 2H), 3.09 (s, 3H), 2.67 (s, 3H), 2.47 (s, 3H), 2.11-2.03 (m, 3H), 1.97-1.91 (m, 1H), 1.89-1.83 (m, 2H), 1.55-1.47 (m, 2H), 1.40-1.29 (m, 4H), 1.24-1.20 (m, 2H), 0.96 (s, 9H).

### Example A298. 2-amino-3-cyano-N-((S)-6-(6-((2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)-2-azaspiro[4.5]decan-8-yl)carbamoyl)pyridin-3-yl)-2,3-dihydrobenzofuran-3-yl)-N-methylquinoline-6-carboxamide

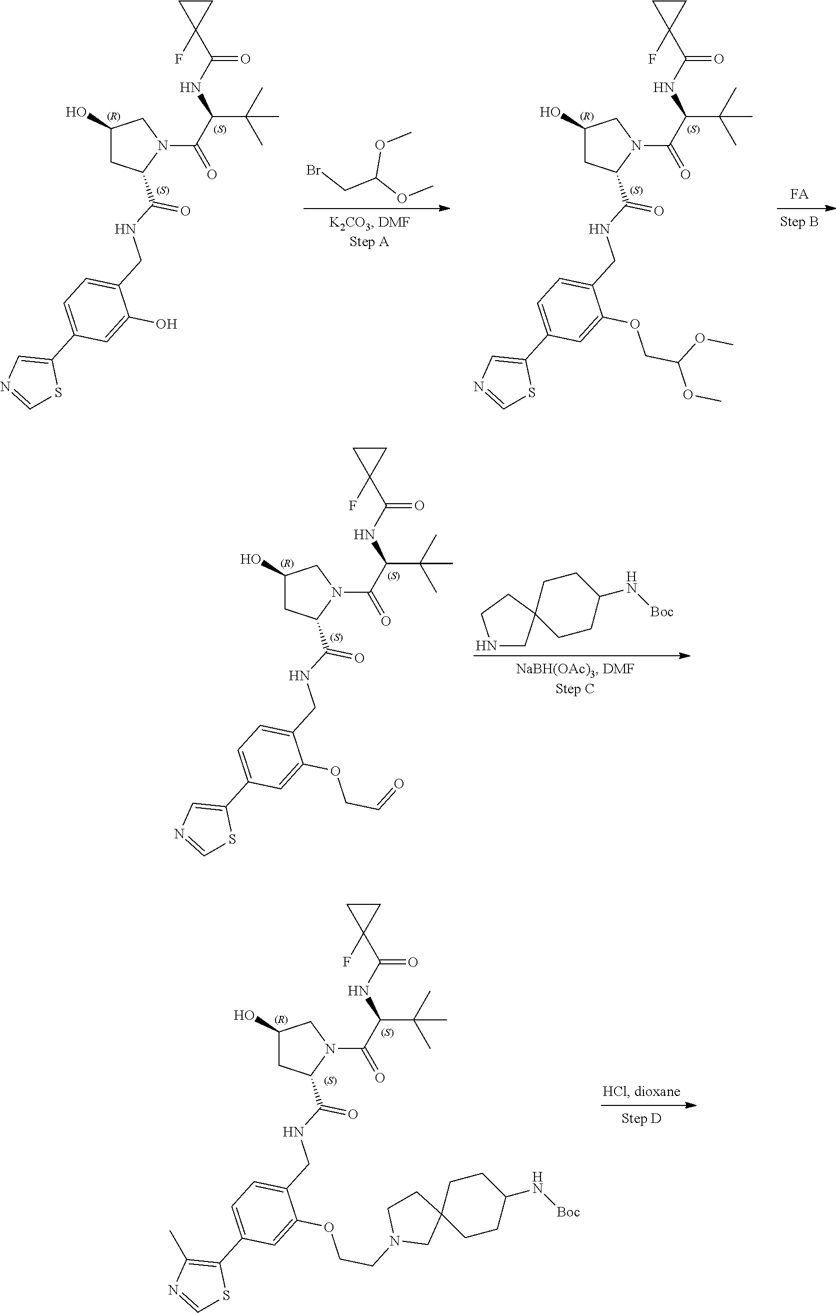

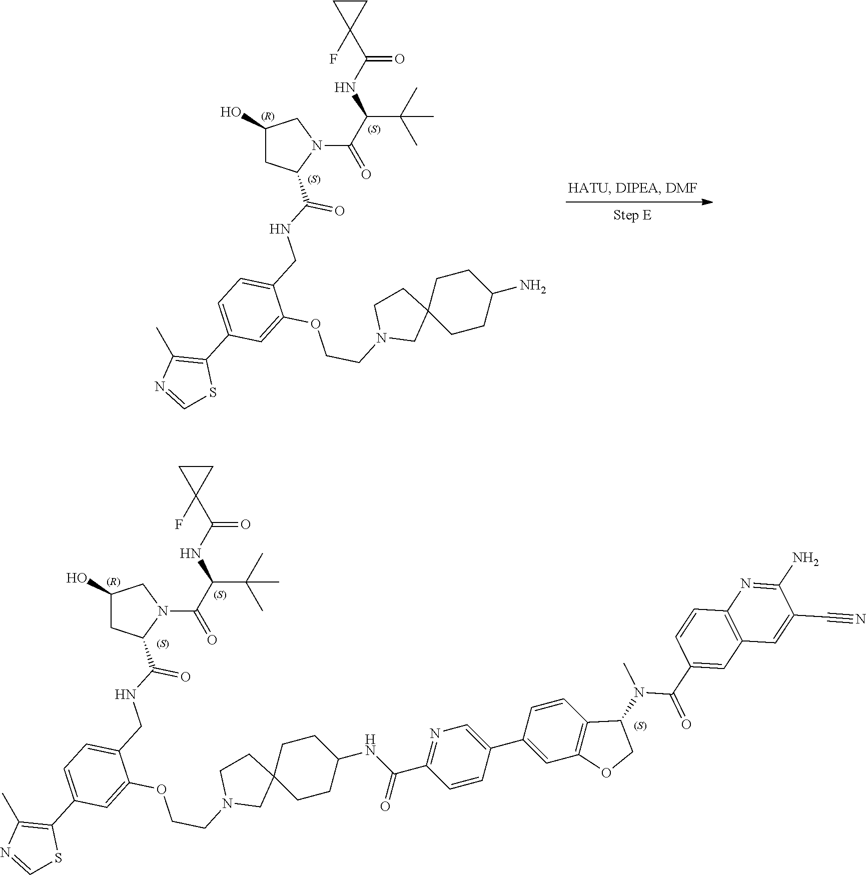

Example A298 was synthesized following the same procedures as described in Example A23 and A107 (2.4 mg, 7% yield over 5 steps) as a white solid. LC-MS (ESI): mass calcd. for C₆₃H₇₀FN₁₁O₈S, 1159.51; m/z found, 1160.1 [M+H]⁺. ¹H NMR (400 MHz, MeOD) δ 8.91-8.85 (m, 2H), 8.62 (s, 1H), 8.23-8.17 (m, 1H), 8.16-8.13 (m, 1H), 7.93 (s, 1H), 7.81 (d, J=8.8 Hz, 1H), 7.71-7.63 (m, 1H), 7.62-7.53 (m, 1H), 7.49-7.46 (m, 1H), 7.35 (d, J=8.3 Hz, 1H), 7.23 (s, 1H), 7.09-7.04 (m, 2H), 4.74-4.68 (m, 3H), 4.64-4.61 (m, 1H), 4.59-4.57 (m, 4H), 4.51-4.46 (m, 1H), 4.40-4.24 (m, 4H), 4.02-3.88 (m, 2H), 3.85-3.77 (m, 2H), 3.07 (s, 1H), 2.94 (s, 1H), 2.79 (s, 3H), 2.50 (s, 3H), 2.25-2.20 (m, 1H), 2.13-2.08 (m, 1H), 1.95-1.91 (m, 3H), 1.88-1.82 (m, 3H), 1.65-1.56 (m, 4H), 1.34-1.26 (m, 4H), 1.00-0.95 (m, 9H).

### Example A299. 2-amino-3-ethynyl-N-((S)-6-(6-((1-(1-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl) azetidin-3-yl)piperidin-4-yl)carbamoyl)pyridin-3-yl)-2,3-dihydrobenzofuran-3-yl)-N-methylquinoline-6-carboxamide

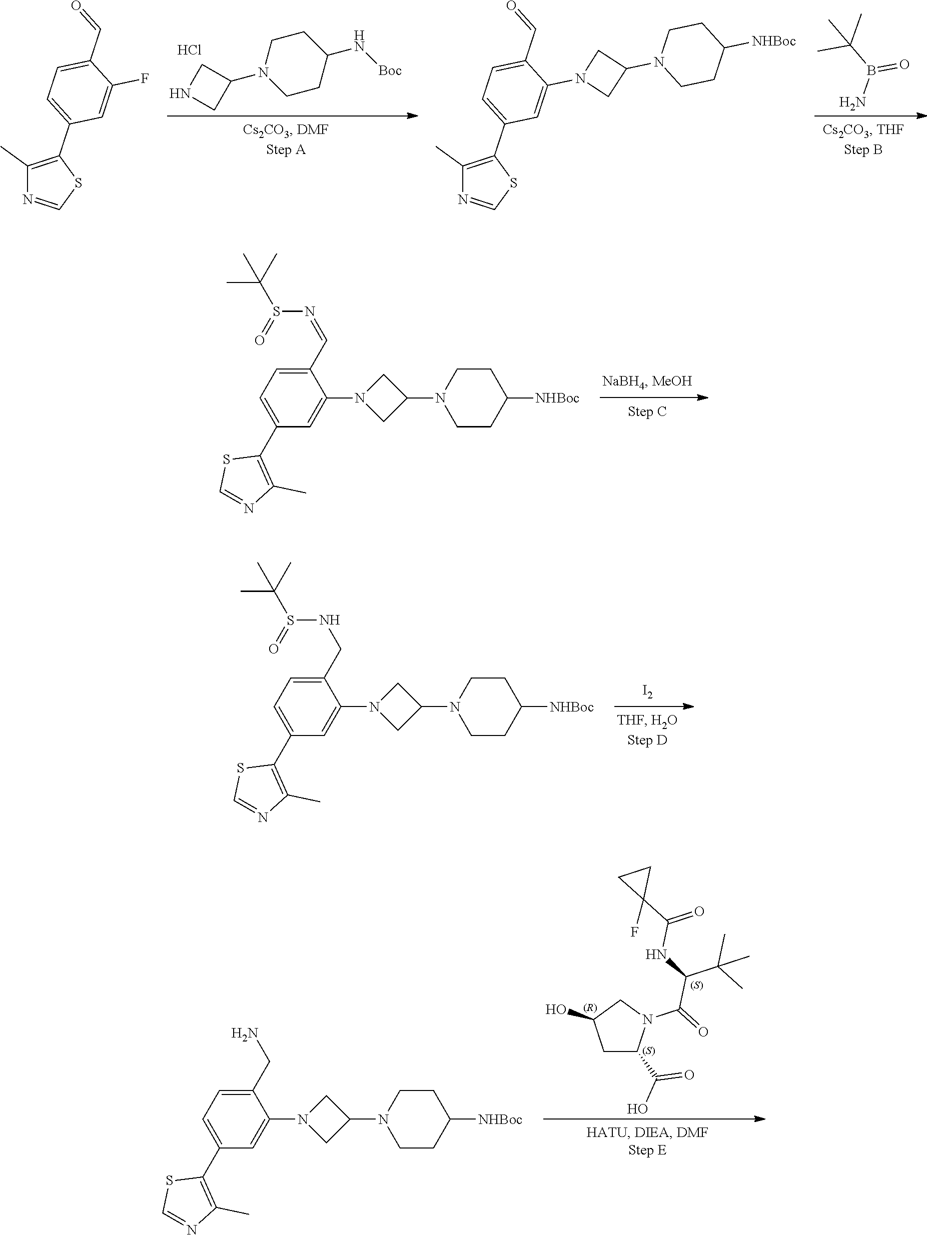

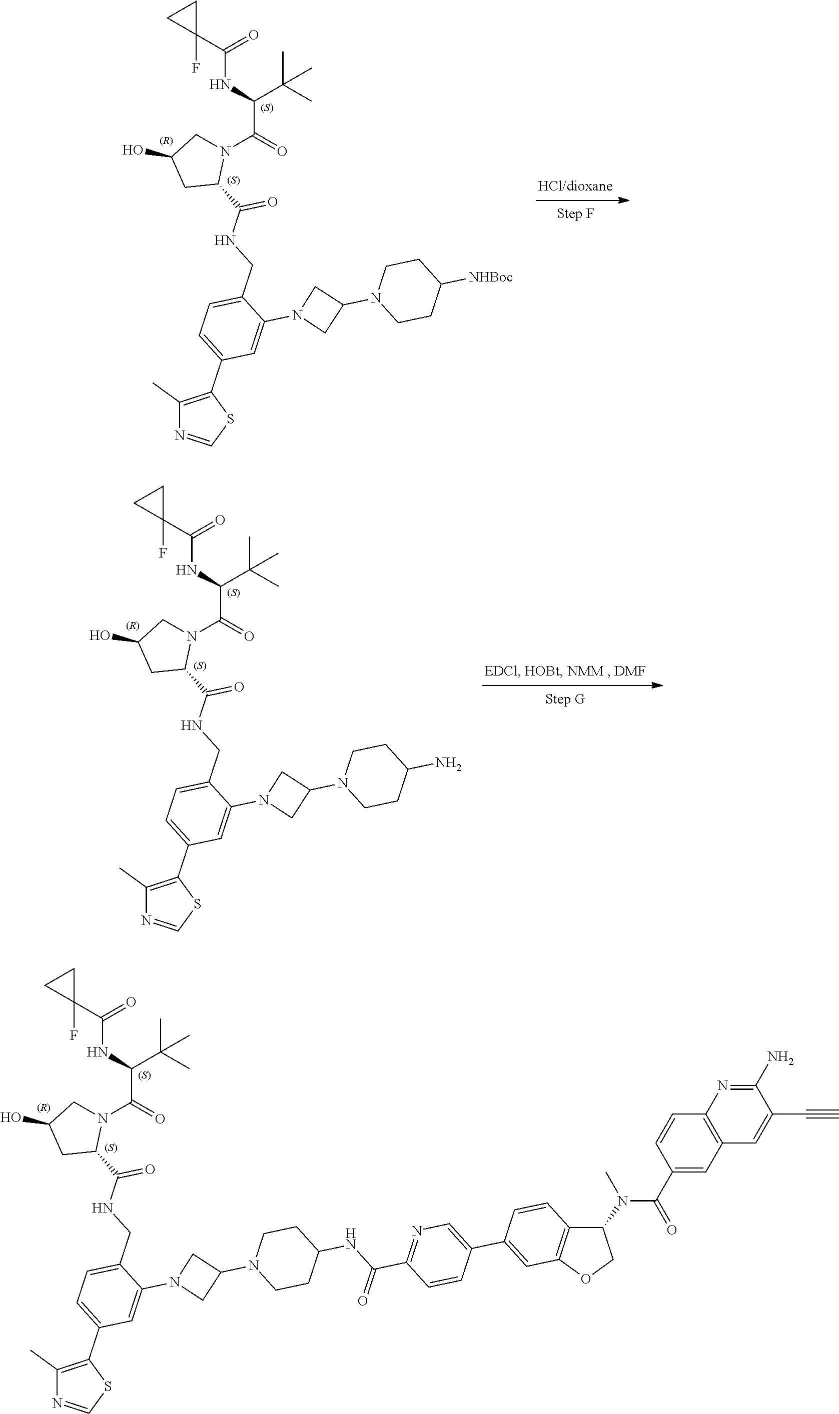

Example A299 was synthesized following the same procedures as described in Example A114 and A107 (2.4 mg, 7% yield over 5 steps) as a white solid. LC-MS (ESI): mass calcd. for C₆₁H₆₆FN₁₁O₇S, 1115.49; m/z found, 1116 [M+H]⁺. ¹H NMR (400 MHz, DMSO-6) δ 8.96 (s, 1H), 8.93 (d, J=1.8 Hz, 1H), 8.61 (d, J=8.6 Hz, 1H), 8.44 (t, J=5.0 Hz, 1H), 8.28 (dd, J=8.4, 1.8 Hz, 2H), 8.08 (d, J=8.2 Hz, 1H), 7.87 (s, 1H), 7.65 (dd, J=8.7, 1.8 Hz, 1H), 7.53 (dd, J=13.2, 7.9 Hz, 2H), 7.39 (d, J=7.8 Hz, 1H), 7.34 (s, 1H), 7.30 (d, J=7.7 Hz, 1H), 7.25 (dd, J=8.9, 2.3 Hz, 1H), 6.82 (d, J=7.8 Hz, 1H), 6.72 (s, 2H), 6.51 (s, 1H), 5.16 (d, J=3.5 Hz, 1H), 4.88-4.65 (m, 2H), 4.63-4.57 (m, 2H), 4.51 (t, J=8.3 Hz, 1H), 4.35 (s, 1H), 4.29-4.21 (m, 1H), 4.14-4.06 (m, 1H), 4.06-3.97 (m, 2H), 3.85 (s, 1H), 3.70 (s, 2H), 3.64-3.58 (m, 1H), 3.31-3.30 (m, 2H), 3.23-3.14 (m, 2H), 2.88-2.77 (m, 2H), 2.67 (s, 3H), 2.46 (s, 3H), 2.10-2.06 (m, 1H), 2.00-1.88 (m, 3H), 1.78 (s, 2H), 1.69 (s, 2H), 1.38-1.30 (m, 2H), 1.22 (dd, J=7.8, 5.2 Hz, 2H), 0.97 (s, 9H).

### Example A301. 2-amino-3-ethynyl-N-((3S)-6-(6-(3-(2′-(((2S,4R)-1-((S)-2-(1-fluorocyclopropane-1-carboxamido)-3,3-dimethylbutanoyl)-4-hydroxypyrrolidine-2-carboxamido)methyl)-5′-(4-methylthiazol-5-yl)-2,3,4,5-tetrahydro-[1,1′-biphenyl]-4-yl)-3,8-diazabicyclo[3.2.1]octane-8-carbonyl)pyridin-3-yl)-2,3-dihydrobenzofuran-3-yl)-N-methylquinoline-6-carboxamide

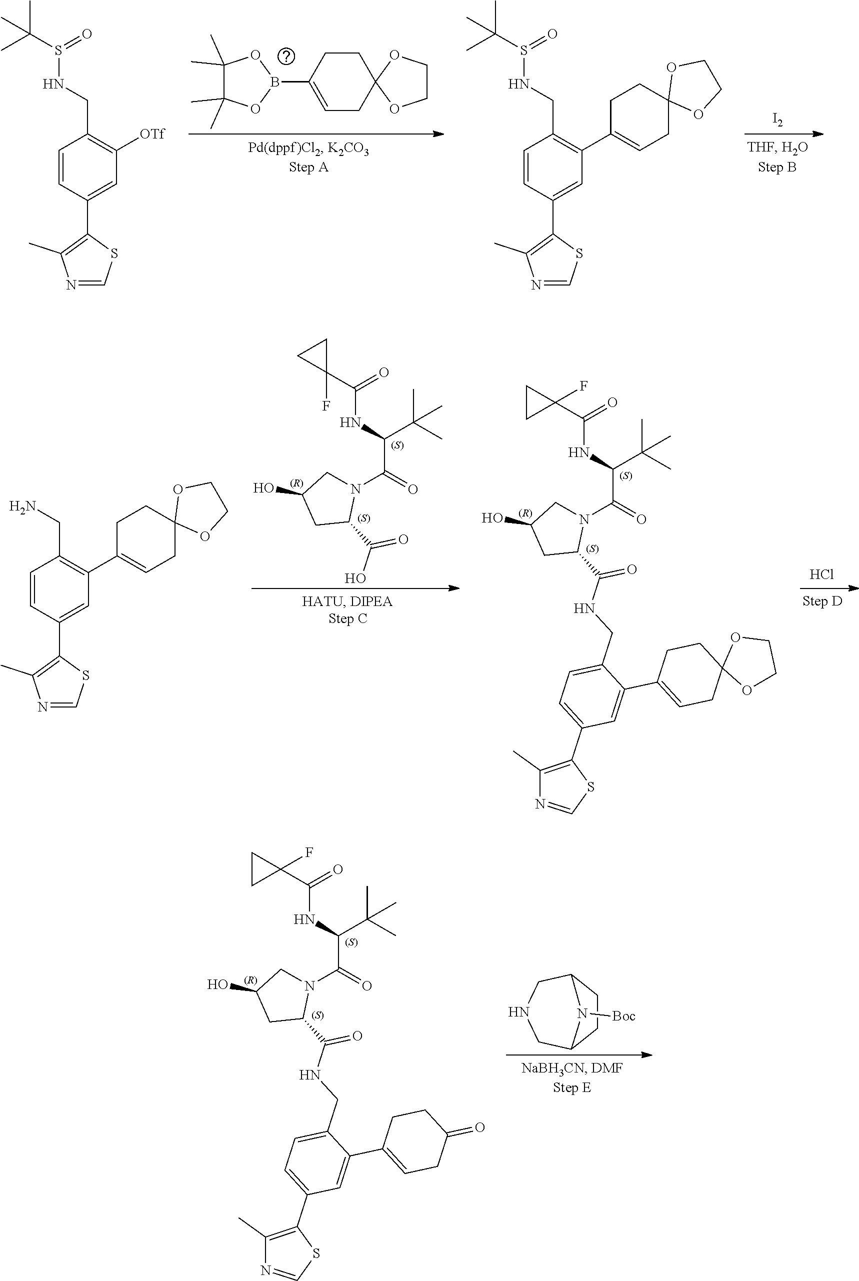

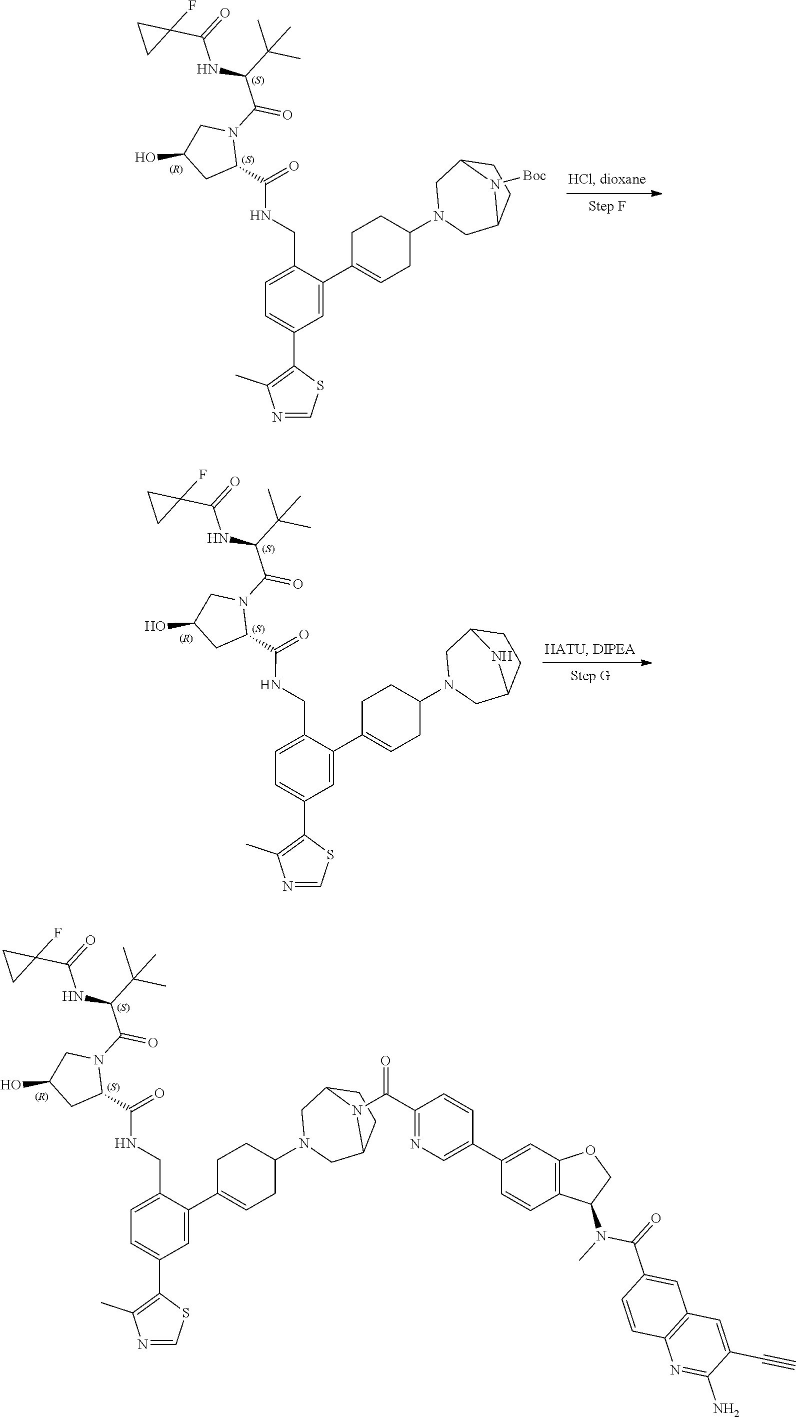

Example A301 was synthesized following the same procedures as described in Example A145 and A154 (4.7 mg, 3% yield over 7 steps) as a white solid. LC-MS (ESI): mass calcd. for C₆₅H₆₉FN₁₀O₇S, 1152.51; m/z found, 1153.7 [M+H]⁺. ¹H NMR (400 MHz, DMSO-d₆) δ 9.00 (d, J=3.2 Hz, 1H), 8.92 (s, 1H), 8.29 (s, 1H), 8.22 (d, J=7.9 Hz, 1H), 7.87 (s, 1H), 7.82-7.75 (m, 1H), 7.65 (d, J=8.6 Hz, 1H), 7.54 (d, J=7.5 Hz, 3H), 7.43-7.29 (m, 5H), 7.27-7.09 (m, 1H), 6.72 (s, 2H), 5.20-5.15 (m, 1H), 5.07-4.88 (m, 2H), 4.86-4.75 (m, 2H), 4.74-4.65 (m, 3H), 4.62 (s, 1H), 4.61-4.33 (m, 3H), 3.71-3.59 (m, 2H), 3.24-3.11 (m, 3H), 2.98-2.89 (m, 1H), 2.67 (s, 3H), 2.47 (d, J=2.9 Hz, 3H), 2.23-2.11 (m, 4H), 1.89-1.73 (m, 4H), 1.43-1.31 (m, 2H), 1.29-1.20 (m, 4H), 1.19-1.09 (m, 2H), 1.01 (d, J=6.7 Hz, 3H), 0.95 (d, J=5.5 Hz, 3H), 0.73 (s, 3H).

### Example A303. 2-amino-3-ethynyl-N-((S)-6-(6-(((1S,4S)-4-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperazin-1-yl)cyclohexyl)carbamoyl) pyridin-3-yl)-2,3-dihydrobenzofuran-3-yl)-N-methylquinoline-6-carboxamide

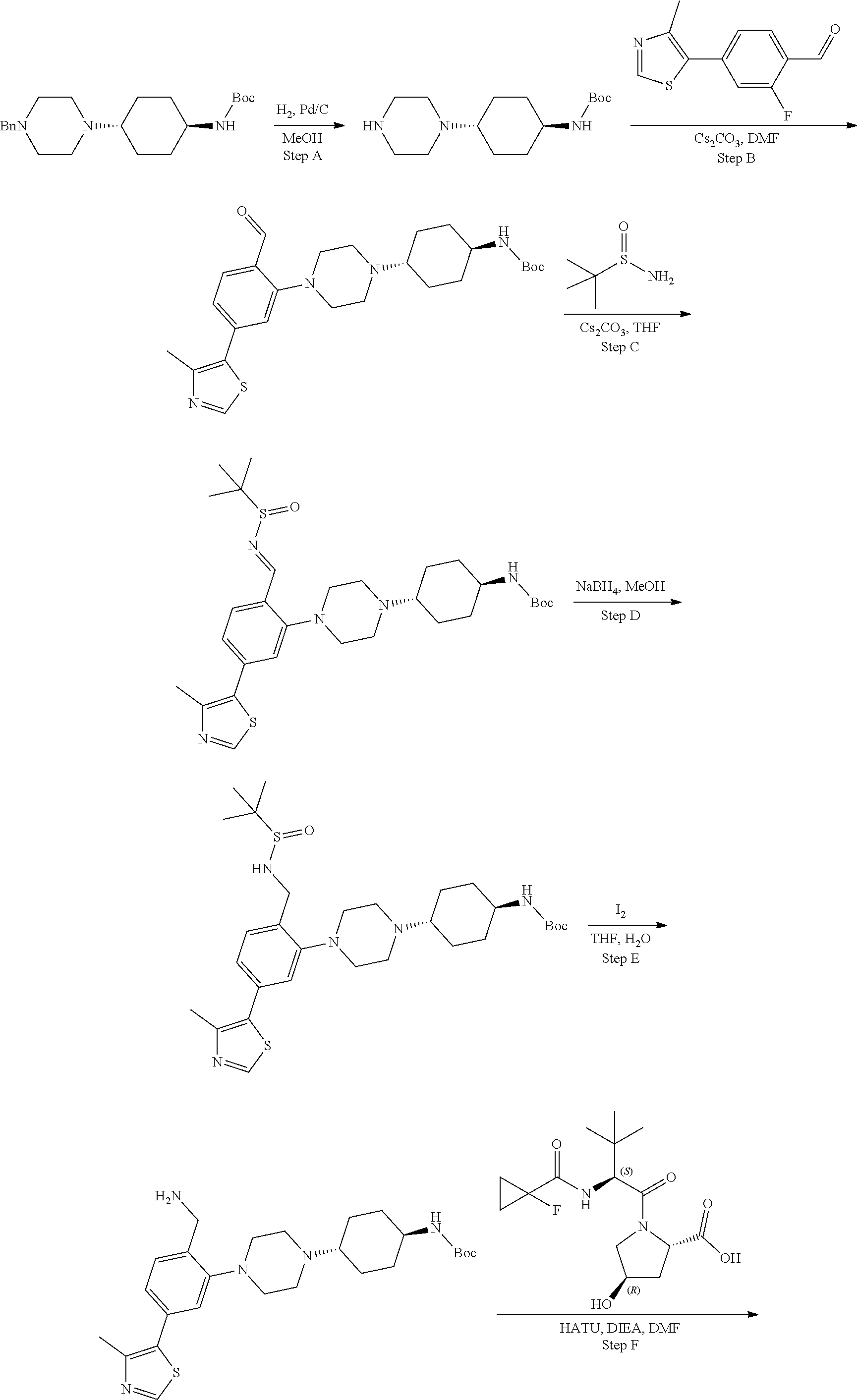

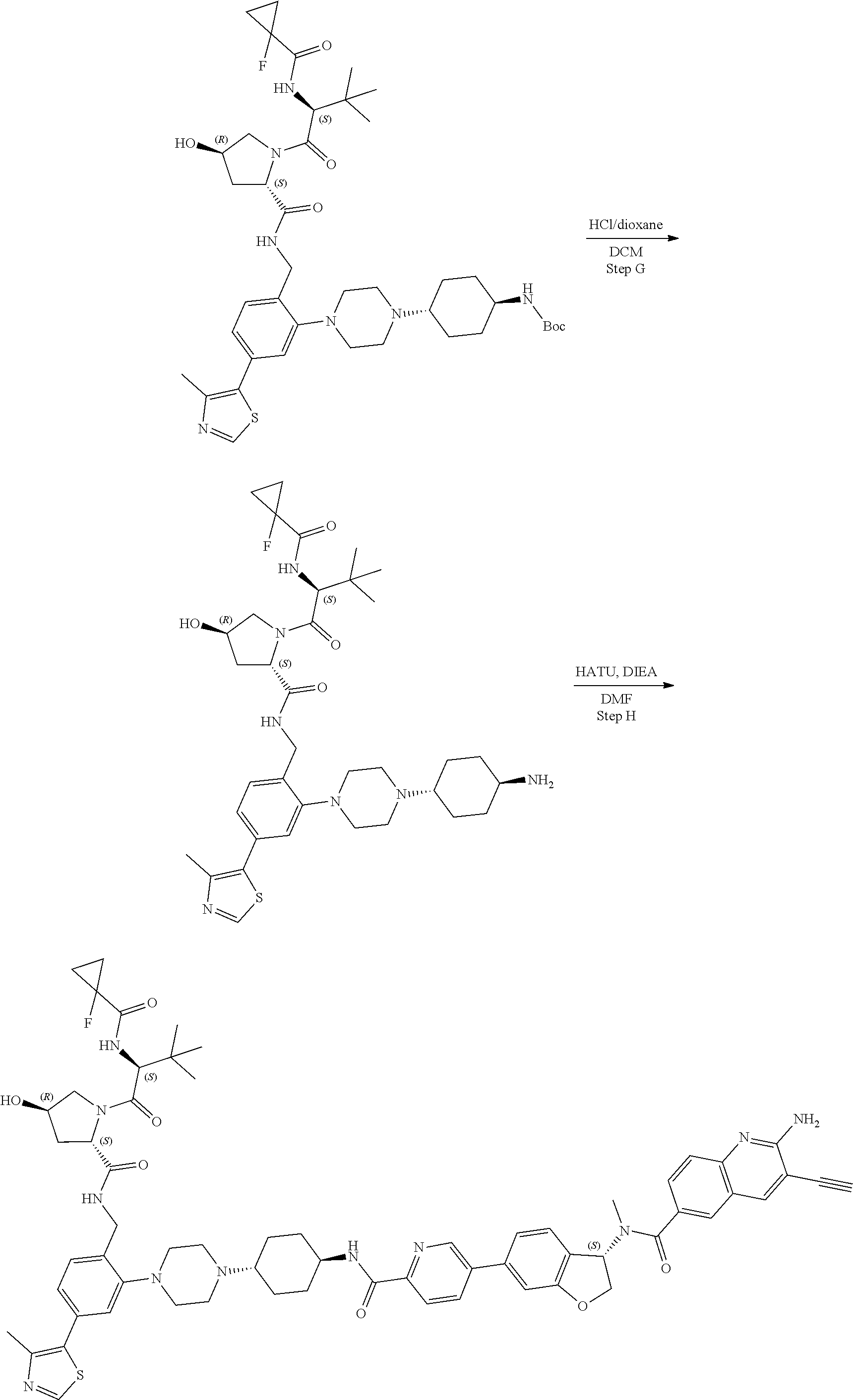

Example A303 was synthesized following the same procedures as described in Example A126 (8.4 mg, 1% yield over 8 steps) as a white solid. LC-MS (ESI): mass calcd. for C₆₃H₇₀FN₁₁O₇S, 1143.5/z found, 1144.2 [M+H]⁺. ¹H NMR (400 MHz, DMSO-d₆) δ 9.52 (s, 1H), 9.02 (s, 1H), 8.94 (s, 1H), 8.71 (d, J=8.5 Hz, 1H), 8.62 (s, 1H), 8.57-8.50 (m, 1H), 8.32 (s, 1H), 8.31-8.27 (m, 1H), 8.10 (d, J=8.2 Hz, 1H), 8.04 (s, 1H), 7.87 (d, J=8.5 Hz, 1H), 7.71 (s, 1H), 7.57 (d, J=7.8 Hz, 1H), 7.50 (d, J=7.9 Hz, 1H), 7.40 (d, J=7.6 Hz, 1H), 7.36 (s, 1H), 7.31-7.24 (m, 1H), 7.22 (d, J=8.1 Hz, 1H), 7.18 (s, 1H), 5.64 (s, 1H), 4.91 (s, 1H), 4.72-4.68 (m, 1H), 4.63-4.60 (m, 1H), 4.52-4.50 (m, 1H), 4.49-4.46 (m, 1H), 4.38-4.36 (m, 1H), 4.34-4.30 (m, 1H), 3.89-3.84 (m, 2H), 3.70-3.55 (m, 5H), 3.38-3.26 (m, 5H), 3.18-3.04 (m, 2H), 2.67 (s, 3H), 2.48 (s, 3H), 2.25-2.15 (m, 2H), 2.14-2.06 (m, 1H), 2.04-1.96 (m, 2H), 1.95-1.87 (m, 1H), 1.74-1.54 (m, 4H), 1.43-1.31 (m, 2H), 1.26-1.19 (m, 2H), 0.99 (s, 9H).

### Example A304. 2-amino-3-ethynyl-N-(S)-6-(5-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyrazin-2-yl)-2,3-dihydrobenzofuran-3-yl)-N-methylquinoline-6-carboxamide

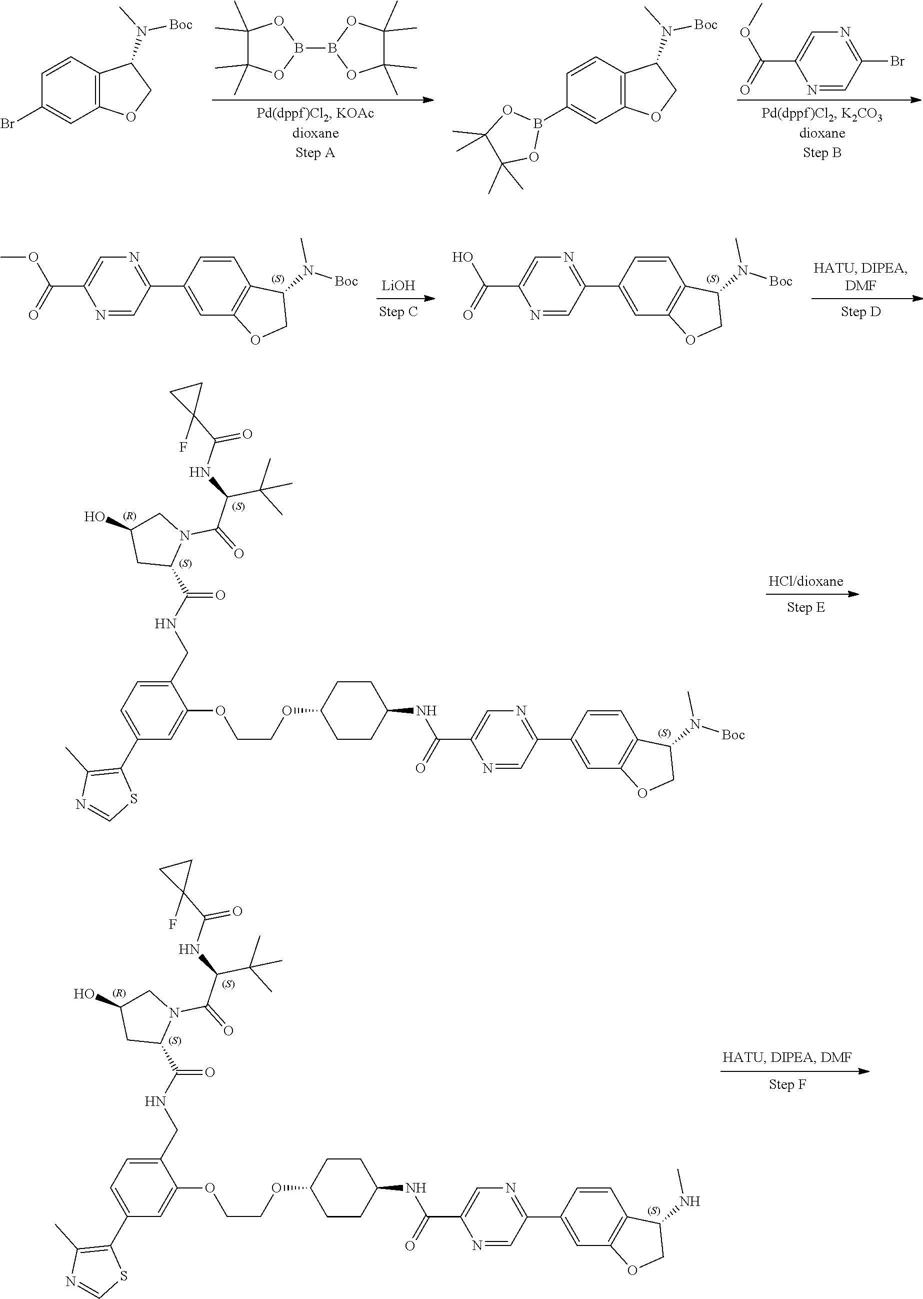

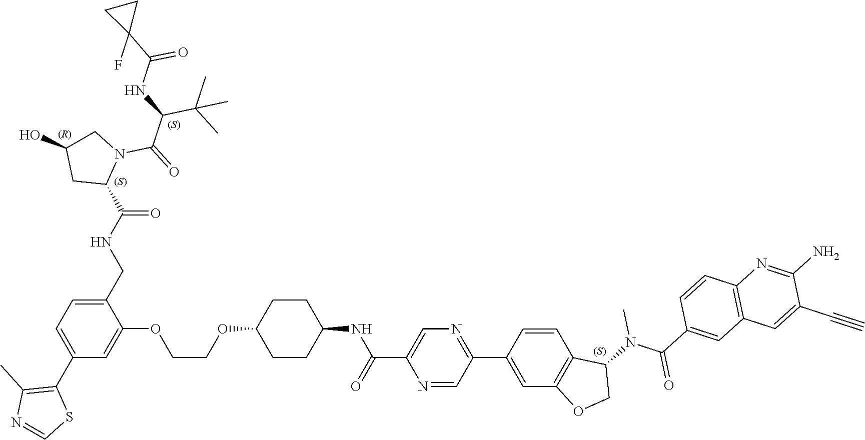

Example A304 was synthesized following the same procedures as described in Intermediate 3 and Example A114 (6.0 mg, 4% yield over 6 steps) as a white solid. LC-MS (ESI): mass calcd, for C₆₀H₆₅FN₁₀O₉S, 1120.46; m/z found, 1121.9 [M+H]⁺. ¹HNMR (400 MHz, DMSO-d₆) δ 9.27 (s, 1H), 9.19 (s, 1H), 8.99 (s, 1H), 8.73-8.51 (m, 3H), 8.49 (t, J=6.0 Hz, 1H), 8.06 (s, 1H), 7.92-7.83 (m, 2H), 7.77-7.67 (m, 2H), 7.60 (d,)=7.8 Hz, 1H), 7.41 (d, J=7.8 Hz, 1H), 7.29 (dd, J=9.2, 2.3 Hz, 1H), 7.05 (s, 1H), 6.97 (d, J=7.6 Hz, 1H), 4.93 (s, 1H), 4.78-4.68 (m, 2H), 4.59 (d, J=9.0 Hz, 1H), 4.52 (t, J=7.7 Hz, 1H), 4.38-4.28 (m, 3H), 4.26-4.15 (m, 4H), 3.84-3.77 (m, 5H), 2.66 (s, 3H), 2.46 (s, 3H), 2.13-2.02 (m, 3H), 1.96-1.89 (m, 1H), 1.89-1.80 (m, 2H), 1.58-1.47 (m, 2H), 1.40-1.27 (m, 4H), 1.23-1.17 (m, 2H), 0.95 (s, 9H).

### Example A305. 2-amino-3-(3,3-difluorocyclobutyl)-N-((S)-6-(6-(((1S,4S)-4-(2-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methylquinoline-6-carboxamide

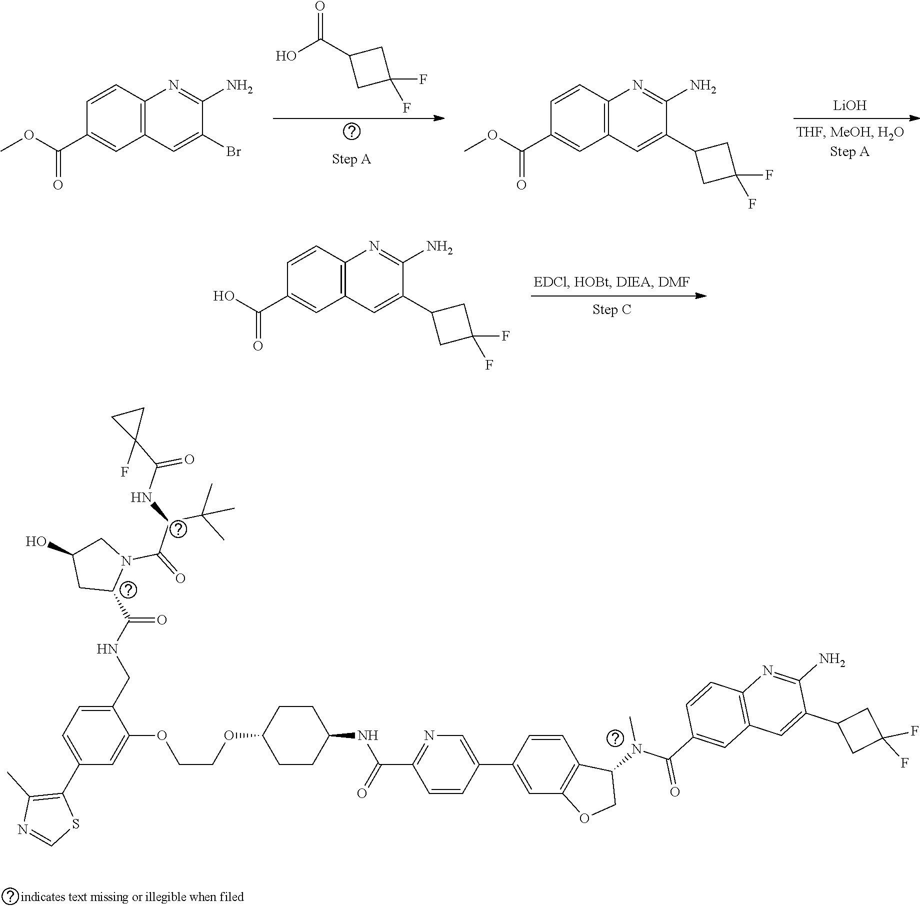

Example A305 was synthesized following the same procedures as described in Example A291 (5.6 mg, 8% yield over 3 steps) as a white solid. LC-MS (ESI): mass calcd. for C₆₃H₇₀F₃NO₉S, 1185.49/z found, 1186.7 [M+H]⁺. ¹H NMR (400 MHz, DMSO-d₆) δ 9.00 (s, 1H), 8.93 (s, 1H), 8.74 (s, 1H), 8.55-8.46 (m, 3H), 8.28 (d, J=8.1 Hz, 1H), 8.15-8.05 (m, 2H), 7.91 (d, J=8.8 Hz, 1H), 7.78 (d, J=7.8 Hz, 1H), 7.56 (d, J=7.7 Hz, 1H), 7.41 (d, J=7.6 Hz, 2H), 7.36 (s, 1H), 7.29 (d, J=7.1 Hz, 1H), 7.06 (s, 1H), 6.97 (d, J=7.9 Hz, 1H), 4.90-4.65 (m, 2H), 4.60 (d, J=9.3 Hz, 1H), 4.52 (t, J=8.1 Hz, 1H), 4.39-4.12 (m, 6H), 3.63-3.57 (m, 4H), 3.52-3.46 (m, 2H), 3.44-3.31 (m, 2H), 3.21-3.13 (m, 2H), 2.93-2.82 (m, 2H), 2.67 (s, 3H), 2.47 (s, 3H), 2.13-2.02 (m, 3H), 1.97-1.90 (m, 1H), 1.89-1.81 (m, 2H), 1.57-1.46 (m, 2H), 1.41-1.27 (m, 4H), 1.24-1.19 (m, 2H), 0.96 (s, 9H).

### Example A306. 2-amino-3-ethynyl-N-((S)-6-(5-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-2-yl)-2,3-dihydrobenzofuran-3-yl)-N-methylquinoline-6-carboxamide

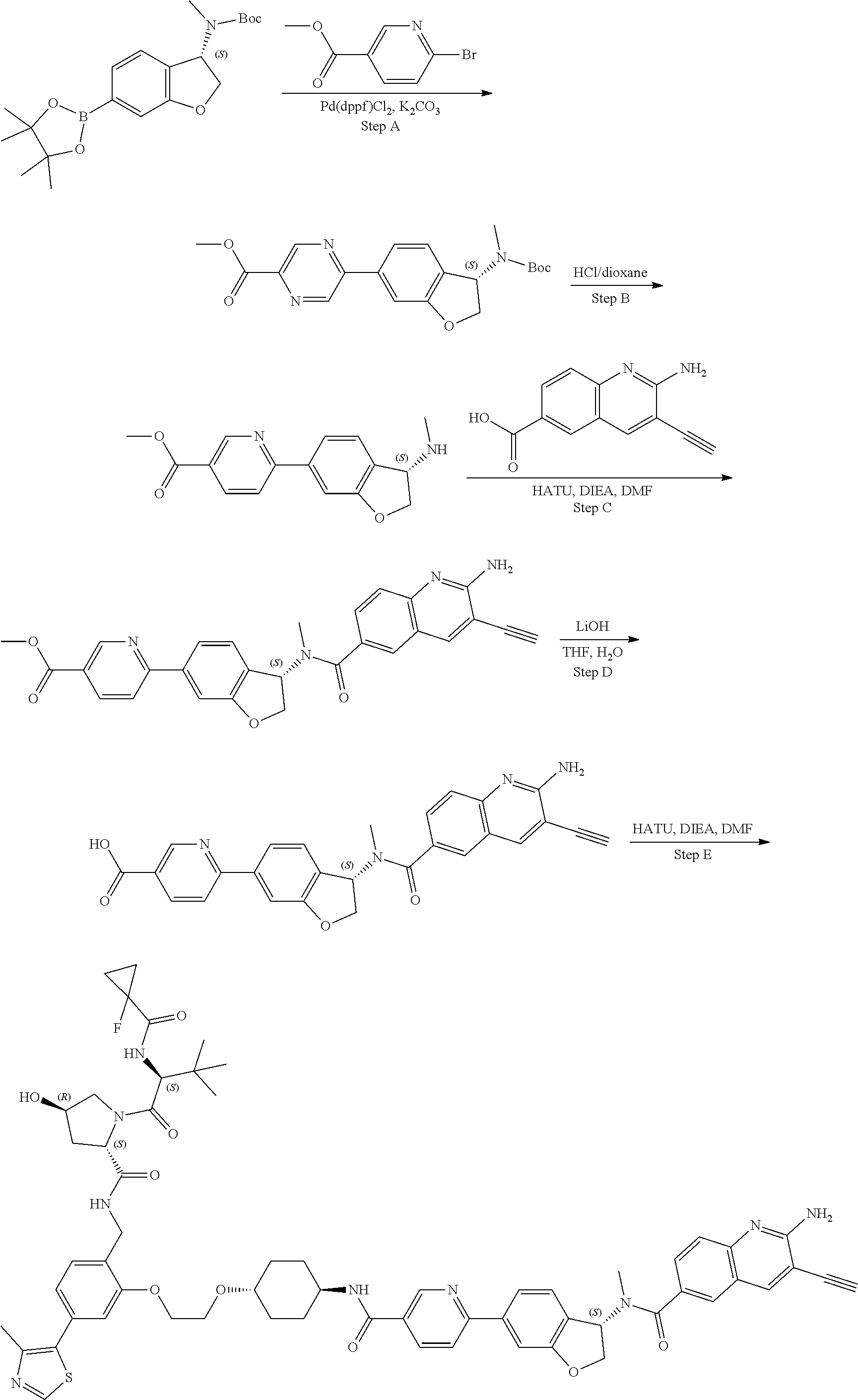

Example A306 was synthesized following the same procedures as described in Intermediate 3 and Example A23 (2.0 mg, 7% yield over 5 steps) as a white solid. LC-MS (ESI): mass calcd, for C₆₁H₆₆FNO₉S, 1119.47; m/z found, 1120.4 [M+H]⁺. ¹H NMR (400 MHz, DMSO-d₆) δ 9.05 (d, J=1.8 Hz, 1H), 9.00 (s, 1H), 8.70 (s, 2H), 8.51 (t, J=5.9 Hz, 1H), 8.45 (d, J=7.6 Hz, 1H), 8.26 (dd, J=8.3, 2.1 Hz, 1H), 8.11-8.04 (m, 2H), 7.92 (dd, J=8.6, 1.5 Hz, 1H), 7.79 (d, J=7.7 Hz, 2H), 7.65 (s, 1H), 7.54 (d, J=7.8 Hz, 1H), 7.42 (d, J=7.8 Hz, 1H), 7.30 (dd, J=9.2, 2.6 Hz, 1H), 7.06 (d, J=1.1 Hz, 1H), 7.00-6.95 (m, 1H), 4.97 (s, 1H), 4.81 (s, 1H), 4.69 (d, J=6.6 Hz, 1H), 4.60 (d, J=9.2 Hz, 1H), 4.53 (t, J=8.2 Hz, 1H), 4.36 (d, J=4.2 Hz, 1H), 4.30 (d, J=6.0 Hz, 1H), 4.25 (d, J=5.7 Hz, 1H), 4.19 (dd, J=8.5, 4.6 Hz, 3H), 3.65 (d, J=3.5 Hz, 2H), 3.62 (s, 1H), 3.42-3.32 (m, 2H), 2.66 (s, 3H), 2.47 (s, 3H), 2.14-2.03 (m, 3H), 1.98-1.85 (m, 3H), 1.46-1.36 (m, 3H), 1.36-1.27 (m, 3H), 1.24-1.19 (m, 2H), 0.96 (s, 9H).

### Example A307. 2-amino-3-ethynyl-N-((3S)-6-(6-(3-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)-3,8-diazabicyclo[3.2.1]octane-8-carbonyl)pyridin-3-yl)-2,3-dihydrobenzofuran-3-yl)-N-methylquinoline-6-carboxamide

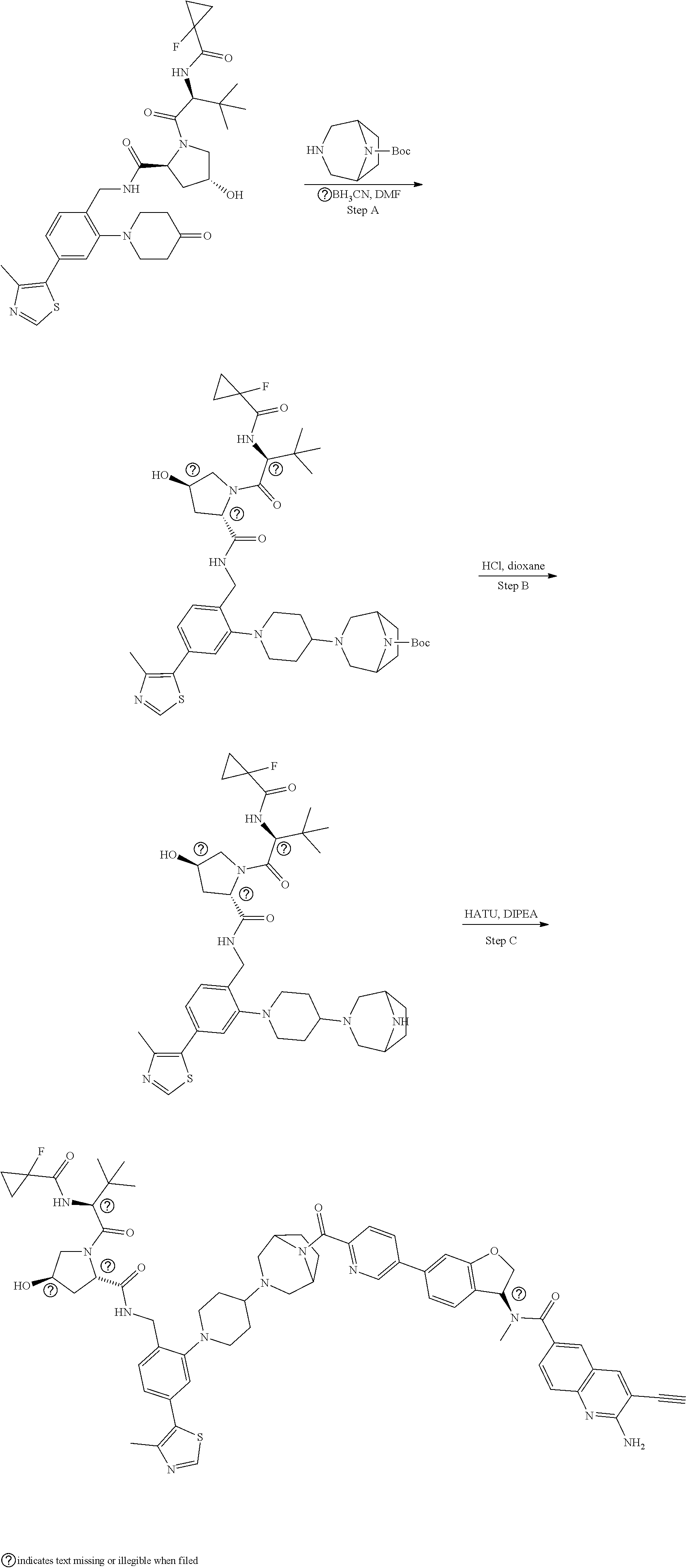

Example A307 was synthesized following the same procedures as described in Example A107 (2.5 mg, 7% yield over 3 steps) as a white solid. LC-MS (ESI): mass calcd. for C₆₄H₇₀FN₁₁O₇S, 1155.52; m/z found, 1156.5 [M+H]⁺. ¹H NMR (400 MHz, DMSO-d₆) δ 9.77-9.59 (m, 1H), 9.00 (s, 1H), 8.95 (d, J=1.4 Hz, 1H), 8.62-8.49 (m, 2H), 8.29 (dd, J=8.2, 2.1 Hz, 1H), 8.15-7.98 (m, 2H), 7.85-7.76 (m, 2H), 7.68 (d, J=8.5 Hz, 1H), 7.56 (d, J=7.8 Hz, 1H), 7.47 (d, J=7.9 Hz, 1H), 7.40 (d, J=7.7 Hz, 1H), 7.35 (s, 1H), 7.27 (d, J=9.1 Hz, 1H), 7.17 (dd, J=8.1, 1.0 Hz, 1H), 7.12 (s, 1H), 4.87 (s, 1H), 4.79 (s, 1H), 4.69 (d, J=6.6 Hz, 1H), 4.60 (d, J=9.4 Hz, 1H), 4.55 (s, 1H), 4.52-4.45 (m, 2H), 4.44 (s, 1H), 4.36 (s, 2H), 4.29 (d, J=16.2 Hz, 2H), 4.16-4.05 (m, 2H), 3.85-3.78 (m, 2H), 3.26-3.13 (m, 4H), 2.83-2.71 (m, 2H), 2.67 (s, 3H), 2.47 (s, 3H), 2.30-2.25 (m, 1H), 2.24-2.17 (m, 2H), 2.14-2.07 (m, 2H), 1.96-1.85 (m, 2H), 1.85-1.75 (m, 2H), 1.41-1.32 (m, 2H), 1.24-1.19 (m, 2H), 0.98 (s, 9H).

### Example A308. 2-amino-3-ethynyl-N-((3S)-6-(6-(3-((15,4r)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)-3,8-diazabicyclo[3.2.1]octane-8-carbonyl)pyridin-3-yl)-2,3-dihydrobenzofuran-3-yl)-N-methylquinoline-6-carboxamide

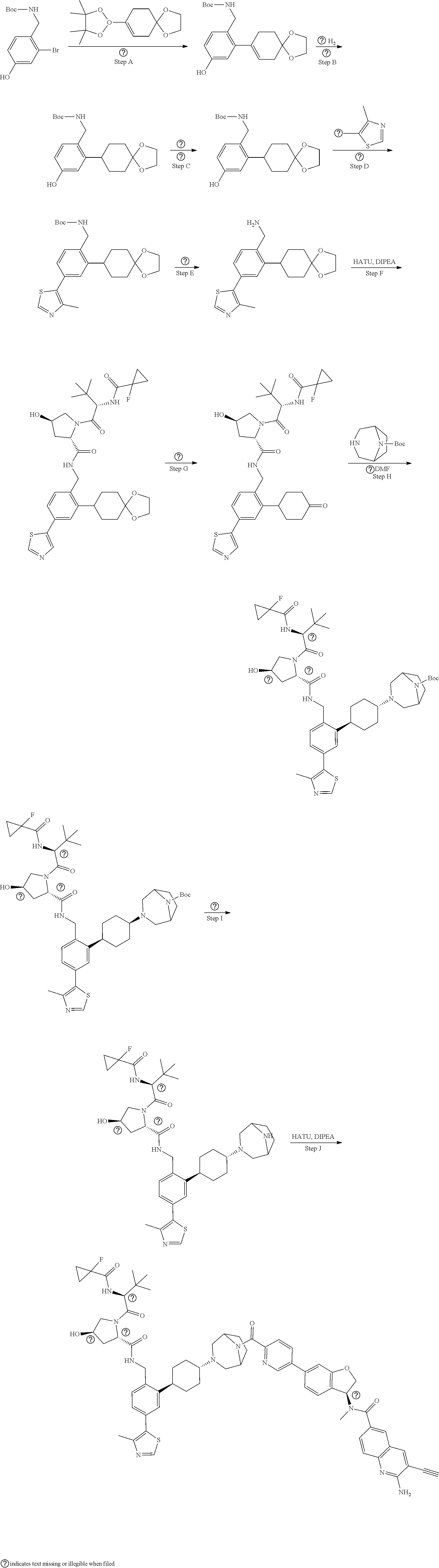

Step A: tert-butyl (4-hydroxy-2-(1,4-dioxaspiro[4.5]dec-7-en-8-yl)benzyl)carbamate

To a solution of tert-butyl(2-bromo-4-hydroxybenzyl)carbamate (3.0 g, 9.9 mmol, 1.0 eq.), 4,4,5,5-tetramethyl-2-(1,4-dioxaspiro[4.5]dec-7-en-8-yl)-1,3,2-dioxaborolane (3.2 g, 11.9 mmol, 1.2 eq.), K₂CO₃ (4.1 g, 29.8 mmol, 3.0 eq.) and PdCl₂ (dppf)-CH2Cl₂ (811 mg, 993 μmol, 0.1 eq.) in 1,4-Dioxane (10 mL) and H₂O (2 mL) was stirred at 90° C. for 6 hours under N₂. The reaction was purified by silica gel chromatography (100-200 mesh silica gel, 0-60% EtOAc in PE) to give tert-butyl (4-hydroxy-2-(1,4-dioxaspiro[4.5]dec-7-en-8-yl)benzyl)carbamate (2.5 g, 69% yield) as a yellow solid. LC-MS (ESI): mass calcd. For C20H₂₇NO_{5, 361.2}; m/z found, 362 [M+H]⁺.

Step B: tert-butyl (4-hydroxy-2-(1,4-dioxaspiro[4.5]decan-8-yl)benzyl)carbamate

To a solution of tert-butyl (4-hydroxy-2-(1,4-dioxaspiro[4.5]dec-7-en-8-yl)benzyl)carbamate (1.0 g, 2.77 mmol, 1.0 eq.) and Pd/C (118 mg, 50% Wt, 553 μmol, 0.5 eq.) in MeOH (15 mL) was stirred at room temperature for 2 hours under H₂. The reaction was filtered and the filtrate was concentrated in vacuo to give the tert-butyl (4-hydroxy-2-(1,4-dioxaspiro[4.5]decan-8-yl)benzyl)carbamate (750 mg, 74% yield) as a yellow solid. LC-MS (ESI): mass calcd. For C₂₀H₂₉NO₅, 363.2; m/z found, 364 [M+H]⁺.

Step C: 4-(((tert-butoxycarbonyl)amino)methyl)-3-(1,4-dioxaspiro[4.5]decan-8-yl) phenyl trifluoromethanesulfonate

To a solution of tert-butyl (4-hydroxy-2-(1,4-dioxaspiro[4.5]decan-8-yl)benzyl)carbamate (750 mg, 2.06 mmol, 1.0 eq.) and pyridine (653 mg, 8.25 mmol, 4.0 eq.) in DCM (15 mL) was cooled to 0° C., the trifluoromethanesulfonic anhydride (873 mg, 3.10 mmol, 1.5 eq.) was slowly added, the reaction was stirred at 0° C. for 2 hours. The reaction was quenched with water (40 mL), extracted with DCM (60 mL), the organic phase was dried over Na₂SO₄, filtered and concentrated in vacuo. The residue was purified by silica gel chromatography (100-200 mesh silica gel, 0-40% EtOAc in PE) to give 4-(((tert-butoxycarbonyl)amino)methyl)-3-(1,4-dioxaspiro[4.5]decan-8-yl) phenyl trifluoromethanesulfonate (800 mg, 78% yield) as a yellow solid. LC-MS (ESI): mass calcd. For C₂₁H₂₈F₃NO₇S, 495.2; m/z found, 440 [M+H−56]⁺.

Step D: tert-butyl (4-(4-methylthiazol-5-yl)-2-(1,4-dioxaspiro[4.5]decan-8-yl)benzyl)carbamate

To a solution of 4-(((tert-butoxycarbonyl)amino)methyl)-3-(1,4-dioxaspiro[4.5]decan-8-yl) phenyl trifluoromethanesulfonate (300 mg, 605 μmol, 1.0 eq.), 4-methyl-5-(4,4,5,5-tetramethyl-1,3,2-dioxaborolan-2-yl) thiazole (164 mg, 727 μmol, 1.2 eq.), K₂CO₃ (251 mg, 1.82 mmol, 3.0 eq.) and PdCl₂ (dppf)-CH₂Cl₂ (49.4 mg, 60.5 μmol, 0.1 eq.) in 1,4-Dioxane (10 mL) and H₂O (2 mL) was stirred at 90° C. for 6 hours under N₂. The reaction was concentrated in vacuo, the residue was purified by silica gel chromatography (100-200 mesh silica gel, 0-30% EtOAc in PE) to give tert-butyl (4-(4-methylthiazol-5-yl)-2-(1,4-dioxaspiro[4.5]decan-8-yl)benzyl)carbamate (190 mg, 70% yield) as a yellow solid. LC-MS (ESI): mass calcd. For C₂₄H32N₂O4S, 444.2; m/z found, 445 [M+H]⁺.

Step E: (4-(4-methylthiazol-5-yl)-2-(1,4-dioxaspiro[4.5]decan-8-yl)phenyl)methanamine

To a solution of tert-butyl (4-(4-methylthiazol-5-yl)-2-(1,4-dioxaspiro[4.5]decan-8-yl)benzyl)carbamate (180 mg, 405 μmol, 1.0 eq.) and HCl/dioxane (4 M/L, 3.0 mL) in DCM (4.0 mL) was stirred at room temperature for 1 hour. The reaction was concentrated in vacuo to give the (4-(4-methylthiazol-5-yl)-2-(1,4-dioxaspiro[4.5]decan-8-yl)phenyl)methanamine (135 mg, 97% yield) as a yellow solid. LC-MS (ESI): mass calcd. For C₁₉H₂₄N₂O₂S, 344.2; m/z found, 345 [M+H]⁺.

Step F: (4-(4-methylthiazol-5-yl)-2-(1,4-dioxaspiro[4.5]decan-8-yl)phenyl)methanamine

To a solution of (4-(4-methylthiazol-5-yl)-2-(1,4-dioxaspiro[4.5]decan-8-yl)phenyl)methanamine (130 mg, 377 μmol, 1.0 eq.), (2S,4R)-1-((S)-2-(1-fluorocyclopropane-1-carboxamido)-3,3-dimethylbutanoyl)-4-hydroxypyrrolidine-2-carboxylic acid (150 mg, 453 μmol, 1.5 eq.) and N-ethyl-N-isopropylpropan-2-amine (146 mg, 1.13 mmol, 3.0 eq.) in DMF (3.0 mL) was added HATU (215 mg, 566 μmol, 1.5 eq.), the reaction was stirred at room temperature for 2 hours. The reaction was concentrated in vacuo, the residue was purified by silica gel chromatography (100-200 mesh silica gel, 0-50% EtOAc in PE) to give (2S,4R)-1-((S)-2-(1-fluorocyclopropane-1-carboxamido)-3,3-dimethylbutanoyl)-4-hydroxy-N-(4-(4-methylthiazol-5-yl)-2-(1,4-dioxaspiro[4.5]decan-8-yl)benzyl)pyrrolidine-2-carboxamide (180 mg, 72% yield) as a yellow solid. LC-MS (ESI): mass calcd. For: C₃₄H₄₅FN₄O₆S,656.3; m/z found, 657 [M+H]⁺.

Step G: (2S, 4R)-1-((S)-2-(1-fluorocyclopropane-1-carboxamido)-3,3-dimethylbutanoyl)-4-hydroxy-N-(4-(4-methylthiazol-5-yl)-2-(4-oxocyclohexyl)benzyl)pyrrolidine-2-carboxamide

To a solution of (2S,4R)-1-((S)-2-(1-fluorocyclopropane-1-carboxamido)-3,3-dimethylbutanoyl)-4-hydroxy-N-(4-(4-methylthiazol-5-yl)-2-(1,4-dioxaspiro[4.5]decan-8-yl)benzyl)pyrrolidine-2-carboxamide (150 mg, 228 μmol, 1.0 eq.) in MeCN (5 mL) and H₂O (1 mL) was added HCl (1.0 mL, 2M, 2.00 mmol, 8.7 eq.). The reaction was stirred at 50° C. for 1 hour. The reaction was quenched with NaHCO₃ (40 mL), extracted with EtOAc (30 mL×3), the organic phase was dried over Na₂SO₄, filtered and concentrated in vacuo. The residue was purified by silica gel chromatography (100-200 mesh silica gel, 0-30% EtOAc in PE) to give (2S,4R)-1-((S)-2-(1-fluorocyclopropane-1-carboxamido)-3,3-dimethylbutanoyl)-4-hydroxy-N-(4-(4-methylthiazol-5-yl)-2-(4-oxocyclohexyl)benzyl)pyrrolidine-2-carboxamide (0.12 g, 86% yield) as a yellow oil. LC-MS (ESI): mass calcd. For: C₃₂H41FN405S, 612.3; m/z found, 613 [M+H]⁺.

Step H: tert-butyl 3-((1S,4r)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)-3,8-diazabicyclo[3.2.1]octane-8-carboxylate

To a solution (2S,4R)-1-((S)-2-(1-fluorocyclopropane-1-carboxamido)-3,3-dimethylbutanoyl)-4-hydroxy-N-(4-(4-methylthiazol-5-yl)-2-(4-oxocyclohexyl)benzyl)pyrrolidine-2-carboxamide (70 mg, 114 μmol, 1.0 eq.), tert-butyl 3,8-diazabicyclo[3.2.1]octane-8-carboxylate (73 mg, 343 μmol, 3.0 eq.) and AcOH (6.86 mg, 114 μmol, 1.0 eq.) in DMF (3 mL) was added Sodium cyanoborohydride (7.18 mg, 114 μmol, 1.0 eq.). The reaction was stirred at room temperature for 16 hours. The reaction was purified by reverse phase purification (C18, MeCN/H2O, 0.1% FA, 0 to 55% gradient, 30 min run) to afford the tert-butyl 3-((1S,4r)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)-3,8-diazabicyclo[3.2.1]octane-8-carboxylate (19.0 mg, 20% yield) as a white solid and tert-butyl 3-((1R,4s)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)-3,8-diazabicyclo[3.2.1]octane-8-carboxylate (20 mg, 21% yield) as a white solid. LC-MS (ESI): mass calcd. For: C43H₆₁FN606S, 808.4; m/z found, 809 [M+H]⁺.

Steps I-J: 2-amino-3-ethynyl-N-((3S)-6-(6-(3-((1S,4r)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)-3,8-diazabicyclo[3.2.1]octane-8-carbonyl)pyridin-3-yl)-2,3-dihydrobenzofuran-3-yl)-N-methylquinoline-6-carboxamide

Example A308 was synthesized following the same procedures as described in Example A29 (4 mg, 16% yield over 2 steps) as a white solid. LC-MS (ESI): mass calcd, for C₆₅H₇₁FN₁₀O₇S, 1154.5; m/z found, 1156 [M+H]⁺. ¹H NMR (400 MHz, DMSO-d₆) δ 9.01 (d, J=2.3 Hz, 1H), 8.95 (s, 1H), 8.74-8.49 (m, 3H), 8.29 (d, J=8.3 Hz, 1H), 8.02 (s, 1H), 7.84 (d, J=8.5 Hz, 1H), 7.82-7.76 (m, 1H), 7.69 (s, 1H), 7.56 (d, J=7.7 Hz, 1H), 7.46 (d, J=6.7 Hz, 2H), 7.40 (d, J=8.5 Hz, 1H), 7.35 (s, 1H), 7.27 (m, 2H), 4.88 (s, 1H), 4.83-4.66 (m, 2H), 4.60 (d, J=9.5 Hz, 1H), 4.49 (t, J=8.1 Hz, 2H), 4.41-4.34 (m, 3H), 4.33-4.22 (m, 2H), 4.05-3.99 (m, 1H), 3.91-3.81 (m, 4H), 3.26 (s, 1H), 3.03 (s, 1H), 2.67 (s, 3H), 2.45 (d, J=4.3 Hz, 3H), 2.23-2.02 (m, 6H), 1.99-1.84 (m, 6H), 1.79-1.69 (m, 2H), 1.41-1.37 (m, 1H), 1.37-1.32 (m, 1H), 1.26-1.20 (m, 3H), 0.97 (s, 9H).

### Example A309. 2-amino-3-ethynyl-N-((3S)-6-(6-(((3S,6R)-6-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl) bicyclo[3.1.0]hexan-3-yl)carbamoyl)pyridin-3-yl)-2,3-dihydrobenzofuran-3-yl)-N-methylquinoline-6-carboxamide

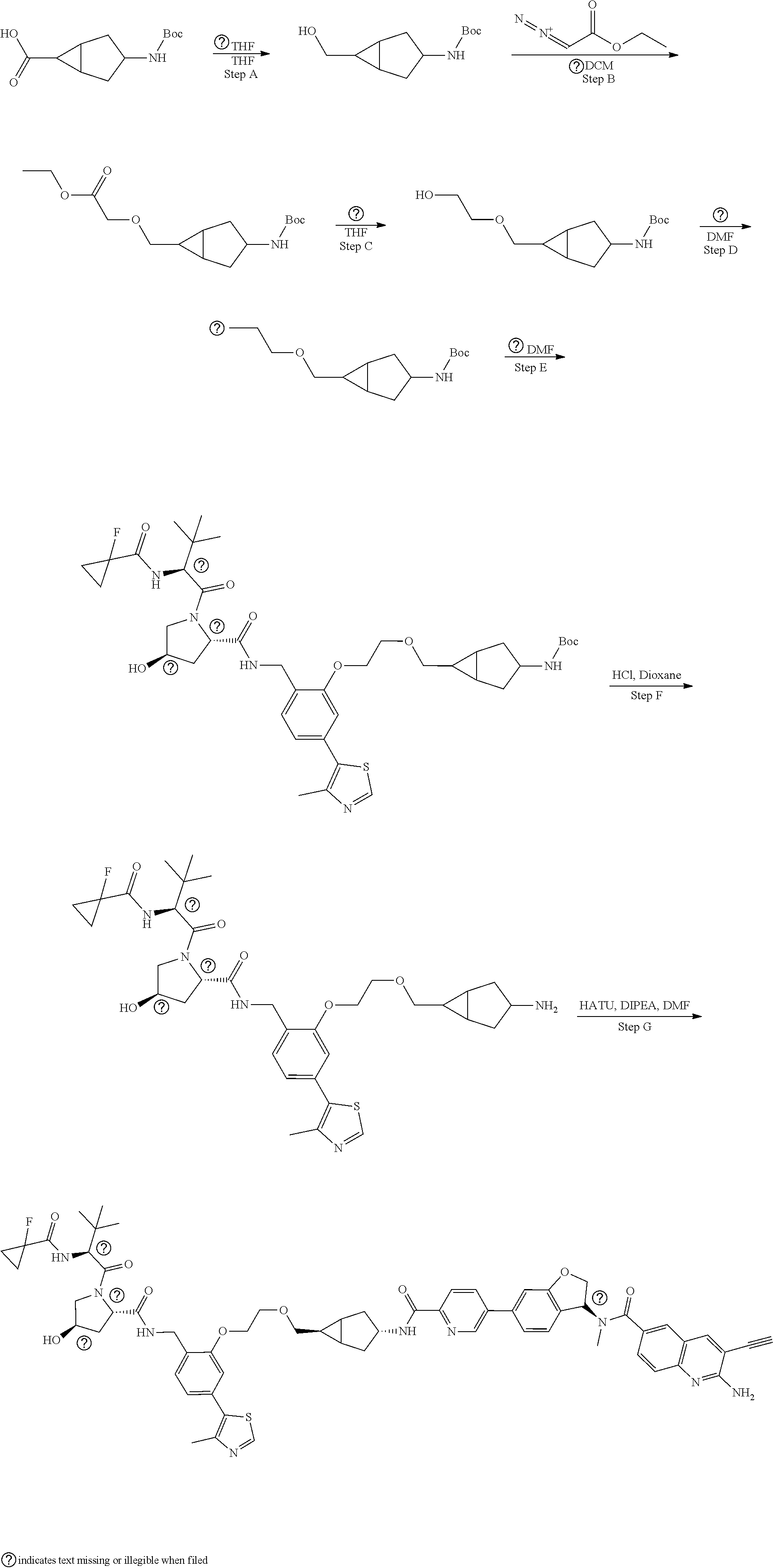

Step A: tert-butyl (6-(hydroxymethyl) bicyclo[3.1.0]hexan-3-yl)carbamate

To a solution of 3-((tert-butoxycarbonyl)amino) bicyclo[3.1.0]hexane-6-carboxylic acid (300 mg, 1.24 mmol, 1.0 eq.) in THF (10 mL) was added dropwise a solution of BH₃. THF (214 mg, 2.49 mmol, 2.0 eq.) at 0° C. The mixture was stirred at 0° C. for 2 hours. The reaction was quenched with methanol (5 mL) and concentrated to afford a residue. The residue was diluted with saturated sodium carbonate (10 mL), extracted with EtOAc (3×20 mL). The combined organic layers were washed with water, dried over anhydrous sodium sulfate, filtered and the filtrate was concentrated to afford tert-butyl (6-(hydroxymethyl) bicyclo[3.1.0]hexan-3-yl)carbamate (100 mg, 35% yield) as a colorless oil. LC-MS (ESI): mass calcd. For C₁₂H₂₁NO₃, 227.09; m/z found, 228.1 [M+H]⁺.

Step B: ethyl 2-((3-((tert-butoxycarbonyl)amino) bicyclo[3.1.0]hexan-6-yl)methoxy)acetate

To a mixture of tert-butyl (6-(hydroxymethyl) bicyclo[3.1.0]hexan-3-yl)carbamate (100 mg, 440 μmol, 1.0 eq.) in DCM (5 mL) was added Rhodium (II) AcetateDimer (10.0 mg, 22.0 μmol, 0.05 eq.) and DAAE (75 mg, 72.5 μL, 660 μmol, 1.5 eq.) at room temperature. The reaction mixture was stirred at room temperature for 16 hours. The resulting mixture was quenched with water (20 mL), diluted with DCM (10 mL×2), and the layers were separated. The organic layer was washed with brine (20 mL) three times, dried over Na₂SO₄, filtered and concentratedconcentrated under vacuum. The crude product was purified by flash chromatography (100-200 mesh silica gel, 0%~20% EtOAc in PE) to afford ethyl 2-((3-((tert-butoxycarbonyl)amino) bicyclo[3.1.0]hexan-6-yl)methoxy)acetate (80.0 mg, 58% yield) as a solid.

LC-MS (ESI): mass calcd. For C₁₆H27NO_{5, 313.19}; m/z found, 314.2 [M+H]⁺.

Step C: tert-butyl (6-((2-hydroxyethoxy)methyl) bicyclo[3.1.0]hexan-3-yl)carbamate

Under an argon atmosphere, ethyl 2-((3-((tert-butoxycarbonyl)amino) bicyclo[3.1.0]hexan-6-yl)methoxy)acetate (80.0 mg, 255 μmol, 1.0 eq.) was added to a vial was charged with LAH (20.0 mg, 511 μmol, 2.0 eq.) in dry THF (mL) at 0° C. The mixture is stirred for 3 hours. The reaction mixture was quenched with NH₄Cl (aq.,10 mL), and then extracted with DCM (20 ml×3). The combined organic layers were washed with brine, dried over anhydrous Na₂SO₄ and concentrated. The residue was purified by column chromatography (silica gel, 0%~10% MeOH in DCM) to afford tert-butyl (6-((2-hydroxyethoxy)methyl) bicyclo[3.1.0]hexan-3-yl)carbamate (50.0 mg, 72% yield) as an oil. LC-MS (ESI): mass calcd. For C14H25NO_{4, 271.18}; m/z found, 272.2 [M+H]⁺.

Steps D-G: 2-amino-3-ethynyl-N-(35)-6-(6-((35,6R)-6-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl) bicyclo[3.1.0]hexan-3-yl)carbamoyl)pyridin-3-yl)-2,3-dihydrobenzofuran-3-yl)-N-methylquinoline-6-carboxamide

Example A309 was synthesized following the same procedures as described in Example A23 (9.6 mg, 29% yield over 4 steps) as a white solid. LC-MS (ESI): mass calcd. for C₆₂H₆₆FN₉O₉S, 1131.47; m/z found, 1132.9 [M+H]⁺. ¹H NMR (400 MHz, DMSO-d₆) δ 8.97 (s, 1H), 8.92 (s, 1H), 8.66 (s, 1H), 8.52-8.40 (m, 2H), 8.28-8.25 (m, 2H), 8.07-8.03 (m, 2H), 7.89 (d, J=8.6 Hz, 1H), 7.78-7.70 (m, 1H), 7.55 (d, J=7.8 Hz, 1H), 7.42-7.37 (m, 2H), 7.34 (s, 1H), 7.28 (d, J=6.7 Hz, 1H), 7.05 (s, 1H), 6.96 (d, J=7.7 Hz, 1H), 4.94 (s, 1H), 4.82-4.66 (m, 2H), 4.58 (d, J=9.2 Hz, 1H), 4.51 (t, J=8.1 Hz, 1H), 4.45-4.40 (m, 1H), 4.35-4.33 (m, 1H), 4.29 (d, J=6.0 Hz, 1H), 4.24 (d, J=5.5 Hz, 1H), 4.21-4.18 (m, 2H), 3.77-3.74 (m, 3H), 3.33 (d, J=6.8 Hz, 4H), 2.66 (s, 3H), 2.45 (s, 3H), 2.29-2.23 (m, 2H), 2.10-2.05 (m, 1H), 1.94-1.88 (m, 1H), 1.76-1.71 (m, 2H), 1.40-1.31 (m, 2H), 1.26-1.24 (m, 2H), 1.23-1.19 (m, 2H), 1.01-0.97 (m, 1H), 0.94 (s, 9H).

### Example A310. 2-amino-3-ethynyl-N-((3S)-6-(6-(((3R,6S)-6-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)methyl) bicyclo[3.1.0]hexan-3-yl)carbamoyl)pyridin-3-yl)-2,3-dihydrobenzofuran-3-yl)-N-methylquinoline-6-carboxamide

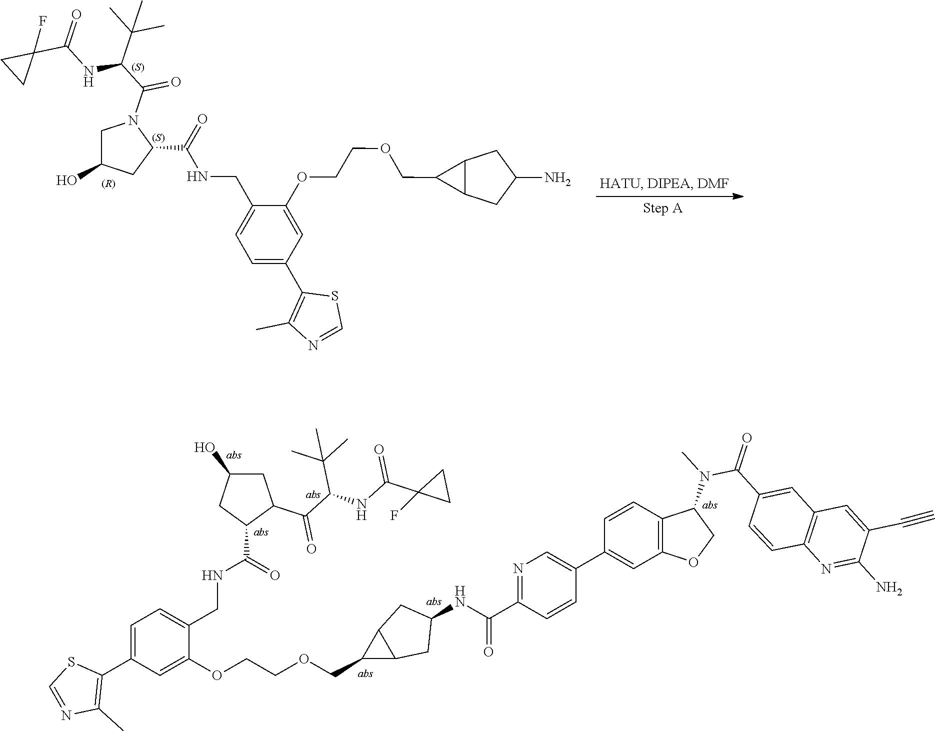

Example A310 was synthesized following the same procedures as described in Example A309 (2.5 mg, 15% yield) as a white solid. LC-MS (ESI): mass calcd, for C₆₂H₆₆FN9O9S, 1131.47; m/z found, 1132.9 [M+H]⁺. ¹H NMR (400 MHz, DMSO-d₆) δ 8.99 (s, 1H), 8.92 (s, 1H), 8.69-8.58 (m, 2H), 8.50 (t, J=5.9 Hz, 1H), 8.39-8.24 (m, 2H), 8.08-8.04 (m, 2H), 7.88 (d, J=9.7 Hz, 1H), 7.77-7.69 (m, 1H), 7.56 (d, J=7.8 Hz, 1H), 7.43-7.37 (m, 2H), 7.35 (s, 1H), 7.31-7.26 (m, 1H), 7.07 (d, J=1.2 Hz, 1H), 6.97 (dd, J=7.6 Hz, 1H), 4.93 (s, 1H), 4.83-4.66 (m, 2H), 4.60 (d, J=9.1 Hz, 1H), 4.54-4.49 (m, 1H), 4.36-4.34 (m, 1H), 4.30 (d, J=5.9 Hz, 1H), 4.24 (d, J=5.5 Hz, 1H), 4.21-4.19 (m, 2H), 4.08-4.03 (m, 1H), 3.78-3.73 (m, 3H), 3.31 (d, J=6.6 Hz, 4H), 2.66 (s, 3H), 2.47 (s, 3H), 2.10-2.01 (m, 3H), 1.96-1.90 (m, 1H), 1.87-1.79 (m, 2H), 1.40-1.32 (m, 2H), 1.24-1.19 (m, 4H), 0.99-0.93 (m, 10H).

### Example A311. 2-amino-3-ethynyl-N-((3S)-6-(6-(3-((1R,4s)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)-3,8-diazabicyclo[3.2.1]-octane-8-carbonyl)pyridin-3-yl)-2,3-dihydrobenzofuran-3-yl)-N-methylquinoline-6-carboxamide

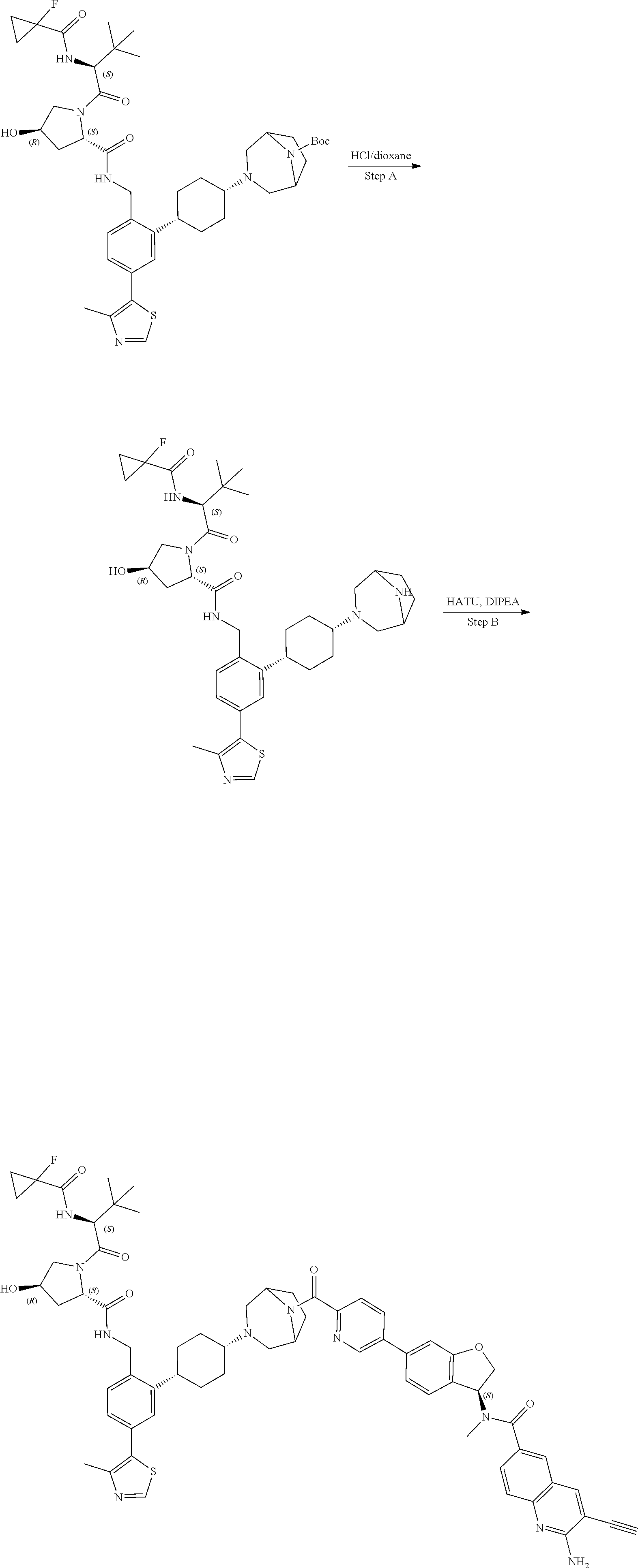

Example A311 was synthesized following the same procedures as described in Example A29 (5 mg, 19% yield over 2 steps) as a white solid. LC-MS (ESI): mass calcd, for C₆₅H₇₁FN₁₀O₇S, 1154.5; m/z found, 1156 [M+H]⁺. ¹H NMR (400 MHz, DMSO-d₆) δ 9.73-9.49 (m, 1H), 9.00 (d, J=3.3 Hz, 1H), 8.95 (s, 1H), 8.63 (s, 1H), 8.51 (s, 1H), 8.38 (s, 1H), 8.31-8.27 (m, 1H), 8.05 (s, 1H), 7.88 (d, J=8.8 Hz, 1H), 7.79 (dd, J=13.6, 8.3 Hz, 1H), 7.72 (s, 1H), 7.56 (d, J=7.7 Hz, 1H), 7.46 (d, J=7.9 Hz, 1H), 7.40 (d, J=7.9 Hz, 1H), 7.36-7.31 (m, 2H), 7.27 (t, J=7.5 Hz, 2H), 4.92 (s, 1H), 4.83-4.68 (m, 2H), 4.60 (d, J=9.2 Hz, 1H), 4.55-4.44 (m, 3H), 4.36 (s, 1H), 4.33-4.27 (m, 2H), 4.16 (s, 1H), 4.07-4.05 (m, 1H), 3.65-3.61 (m, 3H), 3.37 (d, J=14.0 Hz, 1H), 3.10 (s, 1H), 2.87-2.77 (m, 1H), 2.67 (s, 3H), 2.46 (s, 3H), 2.46 (d, J=6.5 Hz, 3H), 2.36-2.14 (m, 4H), 2.13-1.98 (m, 3H), 1.97-1.82 (m, 4H), 1.74-1.47 (m, 4H), 1.41-1.30 (m, 2H), 1.24-1.18 (m, 2H), 0.97 (s, 9H).

### Example A312. 2-amino-3-ethynyl-N-((3S)-6-(6-(3-((4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)cyclohexyl)methyl)-3,8-diazabicyclo[3.2.1]octane-8-carbonyl)pyridin-3-yl)-2,3-dihydrobenzofuran-3-yl)-N-methylquinoline-6-carboxamide

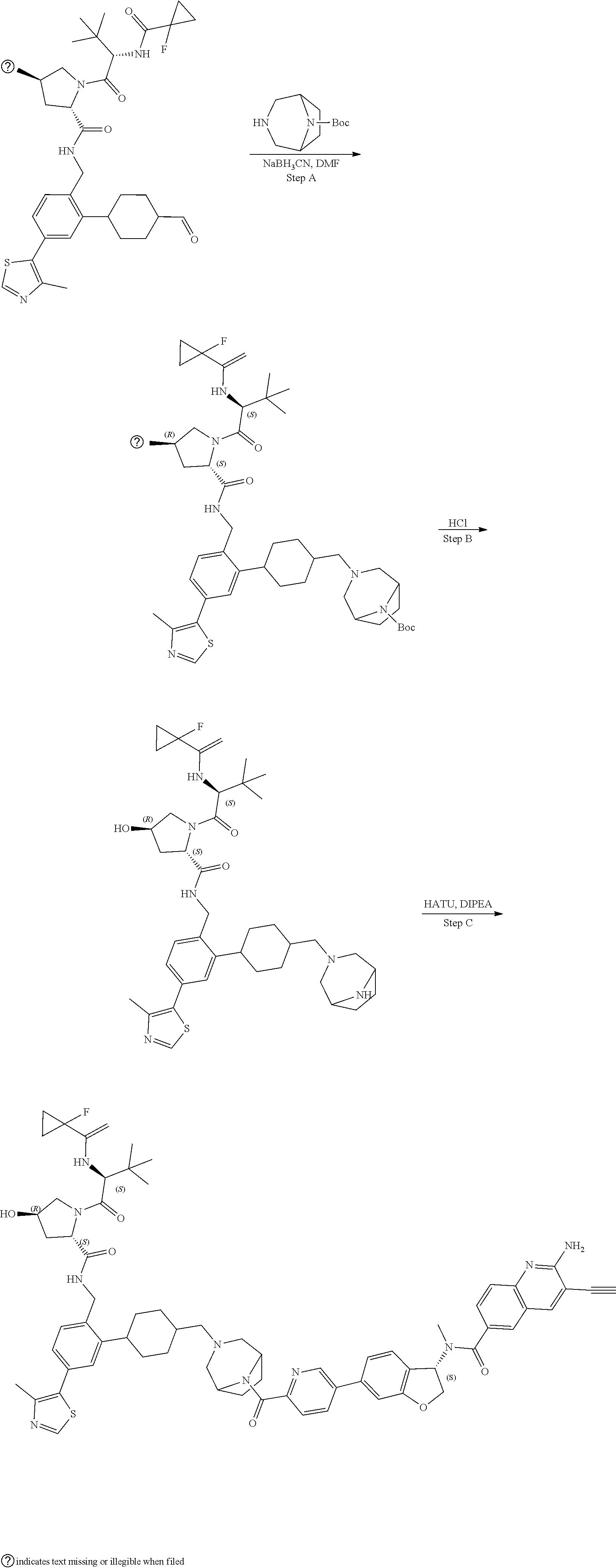

Example A312 was synthesized following the same procedures as described in Example A145 (4.8 mg, 7% yield over 3 steps) as a white solid. LC-MS (ESI): mass calcd. for C₆₆H₇₃FN₁₀O₇S, 1168.54; m/z found, 1169.4 [M+H]⁺. ¹H NMR (400 MHz, DMSO-d₆) δ 8.98 (s, 1H), 8.92 (s, 1H), 8.50-8.43 (m, 1H), 8.30 (s, 1H), 8.23 (dd, J=8.2, 2.2 Hz, 1H), 7.88 (s, 1H), 7.80 (d, J=8.1 Hz, 1H), 7.66 (dd, J=8.6, 1.9 Hz, 1H), 7.53 (t, J=8.1 Hz, 2H), 7.41 (t, J=8.4 Hz, 2H), 7.34 (s, 2H), 7.27-7.21 (m, 2H), 6.73 (s, 2H), 5.15 (d, J=3.5 Hz, 1H), 4.76 (s, 1H), 4.66 (s, 3H), 4.63 (s, 1H), 4.59 (d, J=9.2 Hz, 1H), 4.48 (t, J=8.1 Hz, 1H), 4.35 (s, 3H), 3.75-3.54 (m, 2H), 3.31-3.28 (m, 2H), 2.85-2.73 (m, 3H), 2.76 (s, 2H), 2.68 (s, 4H), 2.45 (d, J=2.9 Hz, 4H), 2.35-2.31 (m, 1H), 2.31-2.22 (m, 3H), 2.19-2.10 (m, 2H), 2.07-2.02 (m, 1H), 1.94-1.84 (m, 6H), 1.81-1.74 (m, 3H), 1.66-1.50 (m, 4H), 1.40-1.37 (m, 1H), 1.36-1.32 (m, 1H), 1.25-1.19 (m, 3H), 0.98 (s, 9H).

### Example A313. 2-amino-3-ethynyl-N-((3S)-6-(6-(3-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)cyclohexyl)-3,8-diazabicyclo[3.2.1]octane-8-carbonyl)pyridin-3-yl)-2,3-dihydrobenzofuran-3-yl)-N-methylquinoline-6-carboxamide

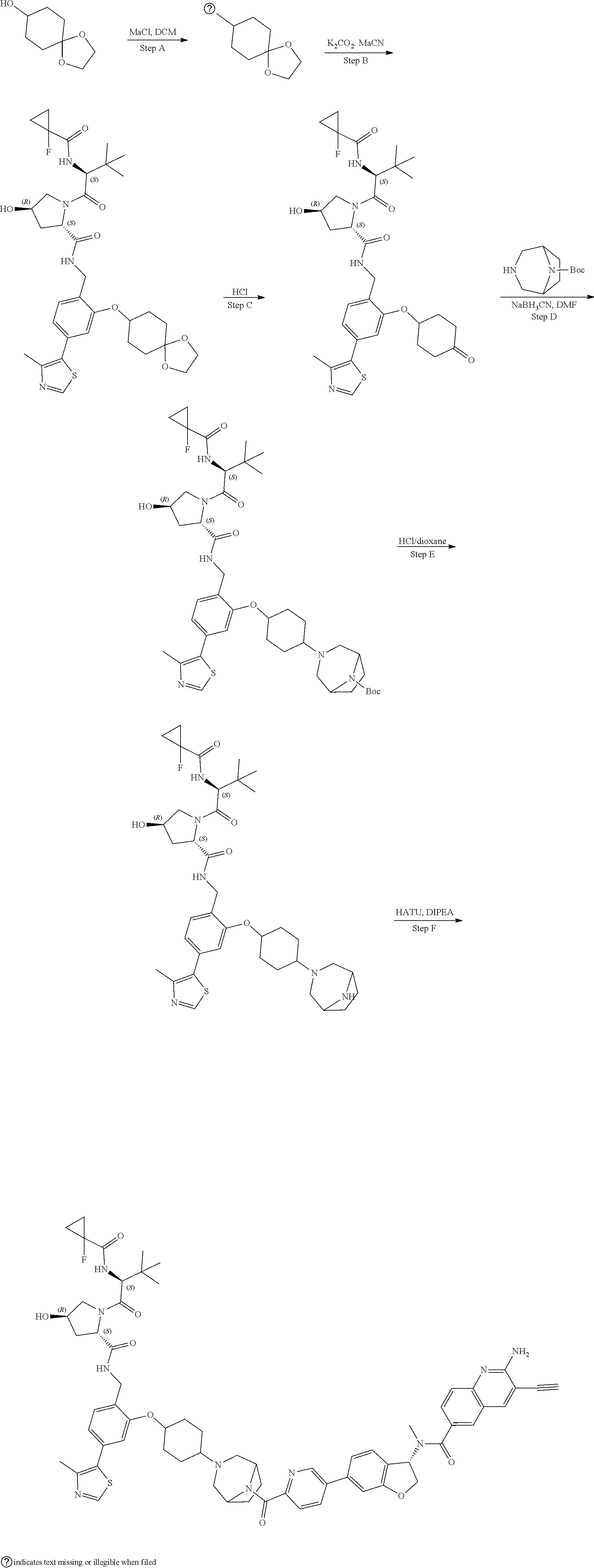

Step A: 1,4-dioxaspiro[4.5]decan-8-yl methanesulfonate

To a stirred mixture of 1,4-dioxaspiro[4.5]decan-8-ol (500 mg, 1.26 mmol, 1.0 eq.) and trimethylamine (640 mg, 2.53 mmol, 2.0 eq.) in DCM (4 mL) was added methanesulfonyl chloride (724 mg, 197 μL, 2.53 mmol, 2.0 eq.) slowly at 0° C. The resulting mixture was stirred at room temperature for 2 hours. The reaction mixture was quenched with water (20 mL), and then extracted with DCM (20 mL×2). The combined organic layers were washed with brine, dried over anhydrous Na₂SO₄ and concentrated to afford 1,4-dioxaspiro[4.5]decan-8-yl methanesulfonate (500 mg, 67% yield) as a white solid. LC-MS (ESI): mass calcd. For C9H1605S, 236.06; m/z found, 237.2 [M+H]⁺.

Step B: (2S,4R)-N-(2-((1,4-dioxaspiro[4.5]decan-8-yl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carbo xamide

To a stirred mixture of (2S,4R)-N-(2-((1,4-dioxaspiro[4.5]decan-8-yl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (100 mg, 188 μmol, 1.0 eq.) and K₂CO₃ (52 mg, 375 μmol, 2.0 eq.) in DMF (3 mL) were added 1,4-dioxaspiro[4.5]decan-8-yl methanesulfonate (177 mg, 751 μmol, 4.0 eq.) at room temperature. The resulting mixture was stirred at 55° C. for 15 hours. The reaction mixture was poured into water (20 mL), and then extracted with EtOAc (20 mL×2). The combined organic layers were washed with brine, dried over anhydrous Na₂SO₄ and concentrated. The residue was purified by column chromatography (silica gel, 0% EtOAc in PE~30% EtOAc in PE) to afford (28,4R)-N-(2-(1,4-dioxaspiro[4.5]decan-8-yl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (90 mg, 71% yield) as a white solid. LC-MS (ESI): mass calcd. For C₃₄H₄₅FN₄O₇S, 672.29; m/z found, 673.4 [M+H]⁺.

Step C: (2S, 4R)-1-((S)-2-(1-fluorocyclopropane-1-carboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide

To a stirred mixture of (2S,4R)-N-(2-((1,4-dioxaspiro[4.5]decan-8-yl)oxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (90 mg, 134 μmol, 1.0 eq.) in Acetonitrile (2 mL) was added 2N HCl (1 mL). The resulting mixture was stirred at 50° C. for 30 minutes. The mixture was concentrated to give (2S,4R)-1-((S)-2-(1-fluorocyclopropane-1-carboxamido)-3,3-dimethylbutanoyl)-4-hydroxy-N-(4-(4-methylthiazol-5-yl)-2-((4-oxocyclohexyl)oxy)benzyl)pyrrolidine-2-carboxamide (80 mg,96% yield) as a white oil. LC-MS (ESI): mass calcd. For C₃₂H41FN406S, 628.27; m/z found, 629.4 [M+H]⁺.

Steps D-F: 2-amino-3-ethynyl-N-(35)-6-(6-(3-(4-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)cyclohexyl)-3,8-diazabicyclo[3.2.1]octane-8-carbonyl)pyridin-3-yl)-2,3-dihydrobenzofuran-3-yl)-N-methylquinoline-6-carboxamide

Example A313 was synthesized following the same procedures as described in Example A107 (3.8 mg, 4% yield over 3 steps) as a white solid. LC-MS (ESI): mass calcd. for C₆₅H₇₁FN₁₀O₈S, 1170.51; m/z found, 1171.4 [M+H]⁺. ¹H NMR (400 MHz, DMSO-d₆) δ 9.53 (s, 1H), 8.99 (d, J=3.3 Hz, 1H), 8.94 (s, 1H), 8.64-8.43 (m, 2H), 8.28 (d,)=8.7 Hz, 1H), 8.01-7.95 (m, 1H), 7.83-7.75 (m, 2H), 7.68-7.61 (m, 1H), 7.56 (d, J=7.9 Hz, 1H), 7.44-7.36 (m, 2H), 7.35 (s, 1H), 7.31-7.26 (m, 1H), 7.10 (d, J=34.8 Hz, 1H), 6.97 (d, J=7.8 Hz, 1H), 4.87-4.73 (m, 2H), 4.71-4.67 (m, 1H), 4.63-4.57 (m, 1H), 4.55-4.50 (m, 1H), 4.49-4.39 (m, 2H), 4.38-4.34 (m, 1H), 4.32-4.22 (m, 2H), 4.09-3.99 (m, 1H), 3.69-3.58 (m, 6H), 3.09-3.01 (m, 2H), 2.67 (s, 3H), 2.47-2.45 (m, 5H), 2.34-2.32 (m, 1H), 2.27-2.17 (m, 3H), 2.14-2.02 (m, 4H), 2.01-1.90 (m, 3H), 1.77-1.70 (m, 1H), 1.60-1.51 (m, 2H), 1.42-1.33 (m, 2H), 0.96 (s, 9H),

### Example A314. 2-amino-N-((S)-6-(6-(((1S,4S)-4-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-3-(2-hydroxyethyl)-N-methylquinoline-6-carboxamide

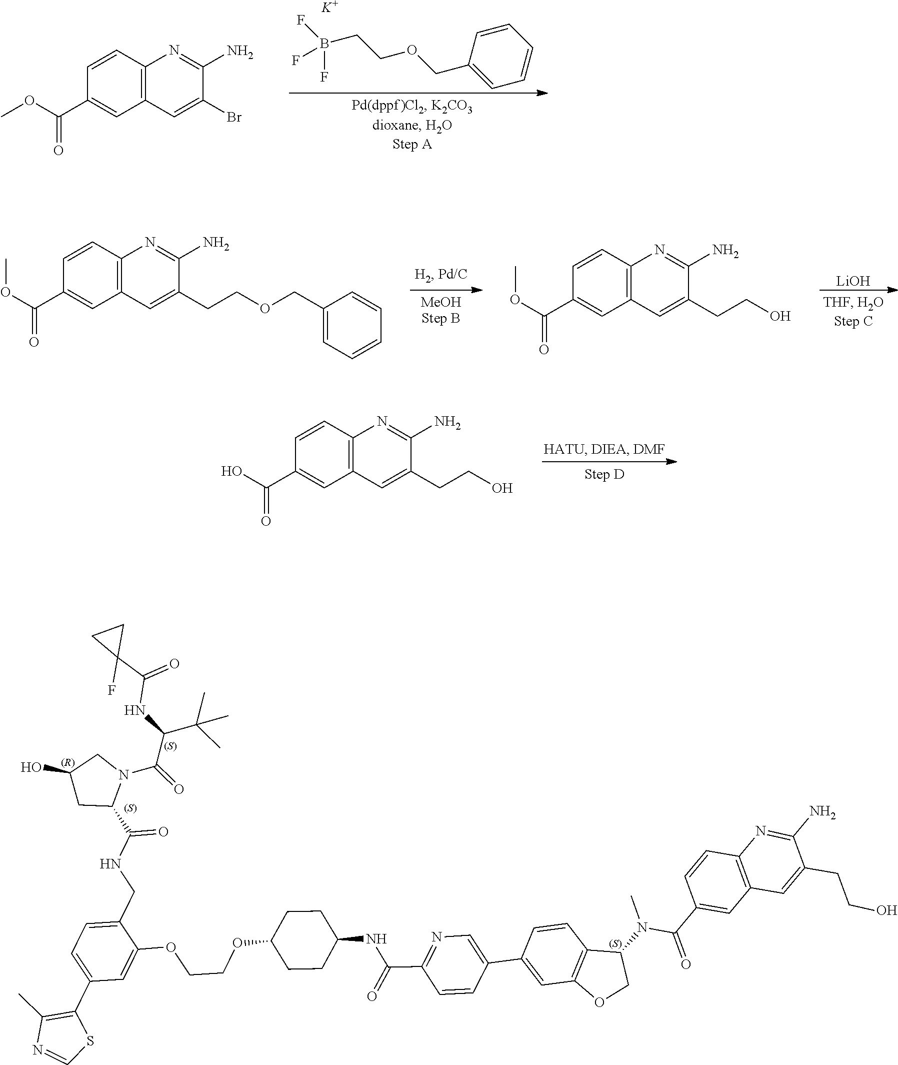

Step A: methyl 2-amino-3-(2-(benzyloxy)ethyl)quinoline-6-carboxylate

To a mixture of methyl 2-amino-3-bromoquinoline-6-carboxylate (300 mg, 1.07 mmol, 1.0 eq.), Potassium (2-benzyloxyethyl) trifluoroborate (388 mg, 1.60 mmol, 1.5 eq.), 1,1′-Bis(diphenylphosphino) ferrocene-palladium (II) dichloride (78.1 mg, 107 μmol, 0.1 eq.) in Toluene (6 mL) and H₂O (2 mL) was added Cs₂CO₃ (1.04 g, 3.20 mmol, 3.0 eq.). The mixture was stirred at 100° C. for 12 hours under N₂ atmosphere. The resulting solution was diluted with 10 mL of water and extracted with EtOAc (30 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford methyl 2-amino-3-(2-(benzyloxy)ethyl)quinoline-6-carboxylate (100 mg, 28% yield) as a yellow oil. LC-MS (ESI): mass calcd. For C₂₀H₂₀N₂O₃, 336.15; m/z found, 337.1 [M+H]⁺.

Step B: methyl 2-amino-3-(2-hydroxyethyl)quinoline-6-carboxylate

To a mixture of methyl 2-amino-3-(2-(benzyloxy)ethyl)quinoline-6-carboxylate (150 mg, 446 μmol, 1.0 eq.), Pd/C (9.49 mg, 89.2 μmol, 0.2 eq.), Pd (OH) 2 (12.5 mg, 89.2 μmol, 0.2 eq.) in MeOH (15.00 mL). The mixture was stirred at 50° C. for 12 hours under H₂ atmosphere. The resulting solution was concentrated under reduced pressure to afford methyl 2-amino-3-(2-hydroxyethyl)quinoline-6-carboxylate (80 mg, 73% yield) as a yellow oil LC-MS (ESI): mass calcd. For C₁₃H₁₄N₂O₃, 246.10; m/z found, 247.3 [M+H]⁺.

Step C: 2-amino-3-(2-hydroxyethyl)quinoline-6-carboxylic acid

To a mixture of methyl 2-amino-3-(2-hydroxyethyl)quinoline-6-carboxylate (20.0 mg, 81.2 μmol, 1.0 eq.), lithium hydroxide (5.84 mg, 244 μmol, 3.0 eq.) in THF (2 mL), H₂O (2 mL). The mixture was stirred at room temperature for 5 hours. The combined organic layers were concentrated under reduced pressure to afford 2-amino-3-(2-hydroxyethyl)quinoline-6-carboxylic acid (10 mg, 53% yield) as a yellow oil, which was used directly without further purification.LC-MS (ESI): mass calcd. For C₁₂H₁₂N₂O₃, 232.02; m/z found, 233.1 [M+H]⁺.

Step D: 2-amino-N-(S)-6-16-(((18,4S)-4-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-3-(2-hydroxyethyl)-N-methylquinoline-6-carboxamide

To a mixture of N-((1S,4S)-4-(2-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-5-((S)-3-(methylamino)-2,3-dihydrobenzofuran-6-yl)picolinamide (8.00 mg, 8.64 μmol, 1.0 eq.), 2-amino-3-(2-hydroxyethyl)quinoline-6-carboxylic acid (3.01 mg, 13.0 μmol, 1.5 eq.), HATU (3.61 mg, 9.50 μmol, 1.1 eq.) in DMF (3 mL) was added DIEA (3.35 mg, 25.9 μmol, 3.0 eq.). The mixture was stirred at room temperature for 2 hours. The mixture was purified by reverse phase purification (C18, MeCN/H2O, 0.1% ammonium bicarbonate modifier, 0 to 95% gradient, 30 mins run) and the fractions containing desired product were lyophilized. The crude product was purified by Prep-HPLC with the condition: Column: YMC-Auctus Triart C18 150×20 mm, 5 μm; Mobile Phase A: Water (0.1% TFA), Mobile Phase B: MeCN; Flow rate: 20 mL/min; Gradient: 5% B to 90% B in 25 min; Wave Length: 254 nm/220 nm nm; RT1 (min): 16.7-18.7 to afford 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-3-(2-hydroxyethyl)-N-methylquinoline-6-carboxamide (3.6 mg, 37% yield) as a white solid. LC-MS (ESI): mass calcd. For C₆₁H₇₀FN₉O₁₀S, 1139.50; m/z found, 1140.50 [M+H]⁺. ¹H NMR (400 MHz, DMSO-d₆) δ 8.99 (s, 1H), 8.92 (s, 1H), 8.67 (s, 2H), 8.49 (dd, J=10.5, 4.7 Hz, 2H), 8.38-8.20 (m, 2H), 8.08 (d, J=8.6 Hz, 2H), 7.87 (d, J=10.1 Hz, 1H), 7.77 (s, 1H), 7.57 (d, J=7.8 Hz, 1H), 7.43-7.32 (m, 3H), 7.32-7.26 (m, 1H), 7.05 (s, 1H), 6.97 (dd, J=6.0, 3.2 Hz, 1H), 4.86-4.67 (m, 2H), 4.59 (d, J=9.3 Hz, 1H), 4.52 (t, J=8.2 Hz, 1H), 4.36 (s, 1H), 4.33-4.22 (m, 2H), 4.18 (s, 2H), 3.80 (d, J=4.3 Hz, 2H), 3.75 (d, J=6.0 Hz, 2H), 3.72 (s, 2H), 3.62 (s, 2H), 3.59 (s, 1H), 3.36 (s, 1H), 2.86 (s, 2H), 2.67 (d, J=2.0 Hz, 3H), 2.47 (s, 3H), 2.11-2.01 (m, 3H), 1.97-1.83 (m, 3H), 1.58-1.45 (m, 2H), 1.41-1.28 (m, 4H), 1.24-1.19 (m, 2H), 0.95 (s, 9H).

### Example A315. N1-(7-amino-2-ethyl-2H-pyrazolo[3,4-c]pyridin-4-yl)-N2-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-N2-methyloxalamide

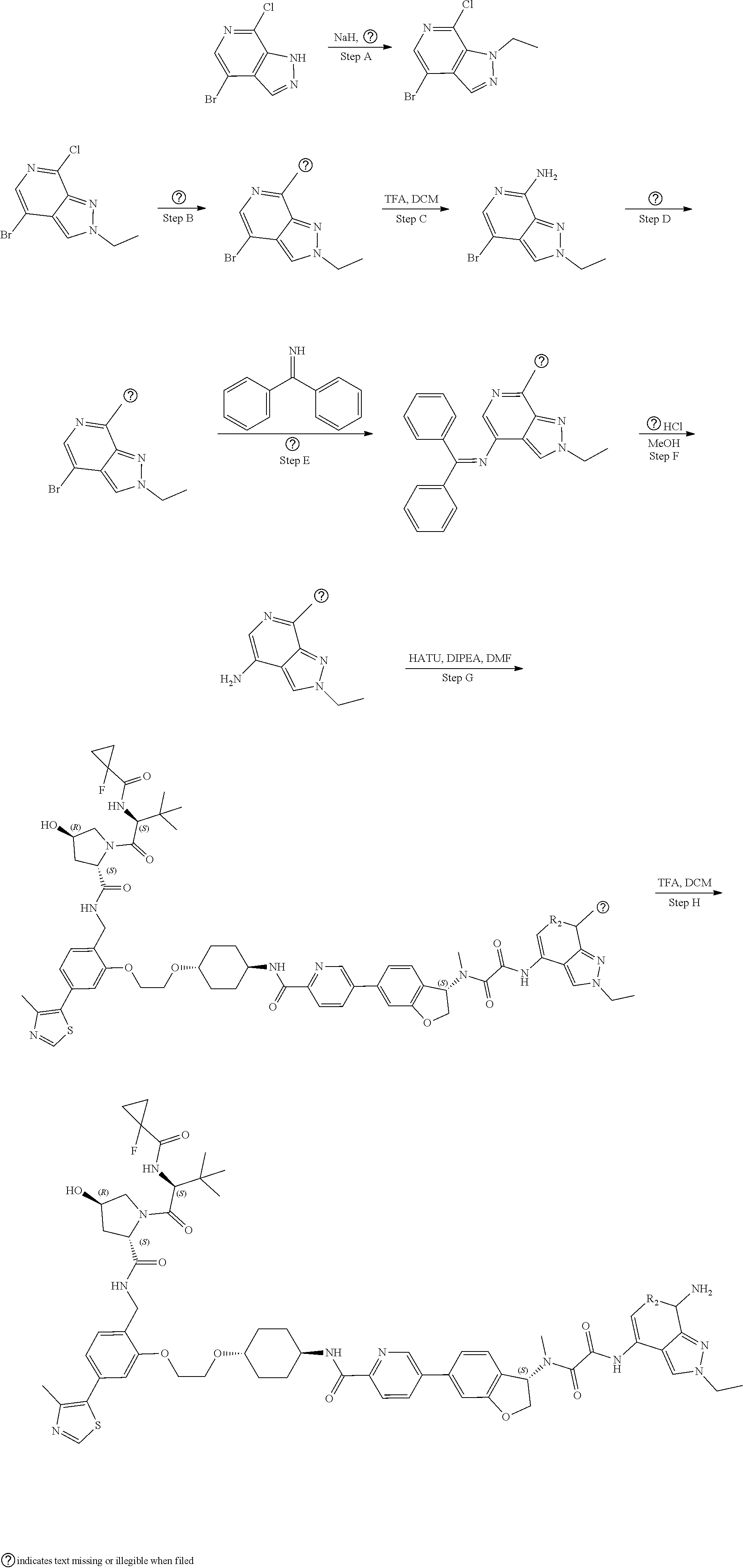

Step A: 4-bromo-7-chloro-2-ethyl-2H-pyrazolo[3,4-c]pyridine

To a solution of 4-bromo-7-chloro-1H-pyrazolo[3,4-c]pyridine (4 g, 17.2 mmol, 1.0 eq.) in DMF (40 mL) was added NaH (2.76 g, 60% Wt, 68.8 mmol, 4.0 eq.). The mixture was stirred at 0° C. for 30 mins, then Ethyl iodide (2.08 mL, 25.8 mmol, 1.5 eq.) was added. The reaction mixture was stirred at room temperature for 3 hours. The reaction mixture was quenched with NH₄Cl (aq. 100 mL), and then extracted with EtOAc (100 mL×2). The combined organic layers were washed with brine three times, dried over anhydrous Na₂SO₄ and concentrated. The residue was purified by column chromatography (silica gel, 0% EtOAc in PE~50% EtOAc in PE) to afford 4-bromo-7-chloro-2-ethyl-2H-pyrazolo[3,4-c]pyridine (1.8 g, 38% yield) as a yellow solid.

LC-MS (ESI): mass calcd. For C₈H₇BrClN₃, 260.5; m/z found, 260.1/262.1 [M+H]⁺.

Step B: 4-bromo-2-ethyl-N-(4-methoxybenzyl)-2H-pyrazolo[3,4-c]pyridin-7-amine

A solution of 4-bromo-7-chloro-2-ethyl-2H-pyrazolo[3,4-c]pyridine (1 g, 3.84 mmol, 1.0 eq.) in PMBNH₂ (3 mL) was stirred at 120° C. for 2 hours. The reaction mixture was quenched with water (100 mL), and then extracted with EtOAc (100 mL×2). The combined organic layers were washed with brine three times, dried over anhydrous Na₂SO₄ and concentrated. The residue was purified by column chromatography (silica gel, 0% EtOAc in PE~100% EtOAc in PE) to afford 4-bromo-2-ethyl-N-(4-methoxybenzyl)-2H-pyrazolo[3,4-c]pyridin-7-amine (1.3 g, 94% yield) as a yellow oil. LC-MS (ESI): mass calcd. For C₁₆H₁₇BrN₄O, 361.2; m/z found, 361.2/363.2 [M+H]⁺

Step C: 4-bromo-2-ethyl-2H-pyrazolo[3,4-c]pyridin-7-amine

A solution of 4-bromo-2-ethyl-N-(4-methoxybenzyl)-2H-pyrazolo[3,4-c]pyridin-7-amine (1.23 g, 3.4 mmol, 1.0 eq.) in TFA (10 mL) was stirred at 60° C. for 16 hours. The reaction mixture was quenched NaHCO₃ solution, and then extracted with EtOAc (200 mL×2). The combined organic layers were washed with brine three times, dried over anhydrous Na₂SO₄ and concentrated. The residue was purified by column chromatography (silica gel, 0% EtOAc in PE~100% EtOAc in PE) to afford 4-bromo-2-ethyl-2H-pyrazolo[3,4-c]pyridin-7-amine (800 mg, 93% yield) as a yellow oil. LC-MS (ESI): mass calcd. For C₈H₉BrN₄, 241.1; m/z found, 241.1/243.1 [M+H]⁺.

Step D: tert-butyl (4-bromo-2-ethyl-2H-pyrazolo[3,4-c]pyridin-7-yl)(tert-butoxycarbonyl)carbamate

To a solution of 4-bromo-2-ethyl-2H-pyrazolo[3,4-c]pyridin-7-amine (800 mg, 3.32 mmol, 1.0 eq.) in DCM (20 mL) was added TEA (1.85 mL, 13.3 mmol, 4.0 eq.), Boc₂O (1.91 mL, 8.30 mmol, 2.5 eq.) and DMAP (81.1 mg, 664 μmol, 0.2 eq) under N₂. The mixture was stirred at room temperature for 4 hours. The reaction mixture was quenched water (50 mL), and then extracted with DCM (50 mL×3). The combined organic layers were washed with brine three times, dried over anhydrous Na₂SO₄ and concentrated. The residue was purified by column chromatography (silica gel, 0% EtOAc in PE~50% EtOAc in PE) to afford tert-butyl (4-bromo-2-ethyl-2H-pyrazolo[3,4-c]pyridin-7-yl)(tert-butoxycarbonyl)carbamate (1.3 g, 71% yield) as a yellow solid.

LC-MS (ESI): mass calcd. For C₁₈H₂₅BrN₄O₄, 441.3; m/z found, 385.3/387.3 [M+H−56]⁺.

Step E: tert-butyl (tert-butoxycarbonyl)(4-((diphenylmethylene)amino)-2-ethyl-2H-pyrazolo[3,4-c]pyridin-7-yl)carbamate

To a solution of tert-butyl (4-bromo-2-ethyl-2H-pyrazolo[3,4-c]pyridin-7-yl)(tert-butoxycarbonyl)carbamate (1 g, 2.27 mmol, 1.0 eq.) in Toluene (10 mL) was added Pd₂(dba): (415 mg, 453 μmol, 0.2 eq.), diphenylmethanimine (616 mg, 3.4 mmol, 1.5 eq.), Cs₂CO₃ (2.21 g, 6.80 mmol, 3.0 eq) and BINAP (564 mg, 906 μmol, 0.4 eq.) under N₂. The mixture was stirred at 110° C. for 3 hours. The reaction mixture was quenched water (50 mL), and then extracted with EtOAc (100 mL×3). The combined organic layers were washed with brine three times, dried over anhydrous Na₂SO₄ and concentrated. The residue was purified by column chromatography (silica gel, 0% EtOAc in PE~50% EtOAc in PE) to afford tert-butyl (tert-butoxycarbonyl)(4-((diphenylmethylene)amino)-2-ethyl-2H-pyrazolo[3,4-c]pyridin-7-yl)carbamate (760 mg, 59% yield) as a yellow solid. LC-MS (ESI): mass calcd. For C₃₁H₃₅N₅O₄, 541.3; m/z found, 641.3 [M+H]⁺.

Step F tert-butyl (4-amino-2-ethyl-2H-pyrazolo[3,4-c]pyridin-7-yl)(tert-butoxycarbonyl)carbamate

A solution of tert-butyl (tert-butoxycarbonyl)(4-((diphenylmethylene)amino)-2-ethyl-2H-pyrazolo[3,4-c]pyridin-7-yl)carbamate (600 mg, 1.11 mmol, 1.0 eq.) in MeOH (5 mL) and 2N HCl (2 mL) was stirred at 0° C. for 1 hour. The reaction mixture was quenched NaHCO₃ solution, and then extracted with EtOAc (100 mL×2). The combined organic layers were washed with brine three times, dried over anhydrous Na₂SO₄ and concentrated. The residue was purified by column chromatography (silica gel, 0% EtOAc in PE~50% EtOAc in PE) to afford tert-butyl (4-amino-2-ethyl-2H-pyrazolo[3,4-c]pyridin-7-yl)(tert-butoxycarbonyl)carbamate (50 mg, 7% yield) as a yellow solid. LC-MS (ESI): mass calcd. For C₁₈H₂₇N₅O₄, 377.2; m/z found, 378.2 [M+H]⁺.

Steps G-H: N1-(7-amino-2-ethyl-2H-pyrazolo[3,4-c]pyridin-4-yl)-N2-((S)-6-(6-(((15,4S)-4-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-N2-methyloxalamide

Example A315 was synthesized following the same procedures as described in Example A44 (1.1 mg, 3% yield over 2 steps) as a white solid. LC-MS (ESI): mass calcd. for C₅₉H₆₉FN₁₂O₁₀S, 1156.5; m/z found, 1157.5 [M+H]⁺. ¹H NMR (400 MHz, DMSO-d₆) δ 12.45 (s, 1H), 11.27-11.00 (m, 1H), 9.16-9.03 (m, 1H), 8.99 (s, 1H), 8.93 (s, 1H), 8.69-8.63 (m, 1H), 8.52-8.46 (m, 2H), 8.31-8.22 (m, 1H), 8.13-8.07 (m, 1H), 7.98-7.84 (m, 1H), 7.60-7.45 (m, 1H), 7.44-7.36 (m, 3H), 7.33-7.25 (m, 1H), 7.05 (s, 1H), 6.97 (d, J=7.5 Hz, 1H), 5.32 (t, J=4.9 Hz, 1H), 4.86-4.65 (m, 2H), 4.63-4.50 (m, 4H), 4.38-4.23 (m, 3H), 4.21-4.14 (m, 2H), 3.85-3.77 (m, 3H), 3.71-3.60 (m, 4H), 2.76-2.65 (m, 3H), 2.47 (s, 3H), 2.05-1.97 (m, 4H), 1.96-1.81 (m, 4H), 1.55 (t, 3H), 1.50-1.42 (m, 2H), 1.41-1.30 (m, 4H), 0.96 (s, 9H).

### Example A316. 2-amino-3-ethynyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-3-oxoisoindolin-5-yl)-2,3-dihydrobenzofuran-3-yl)-N-methylquinoline-6-carboxamide

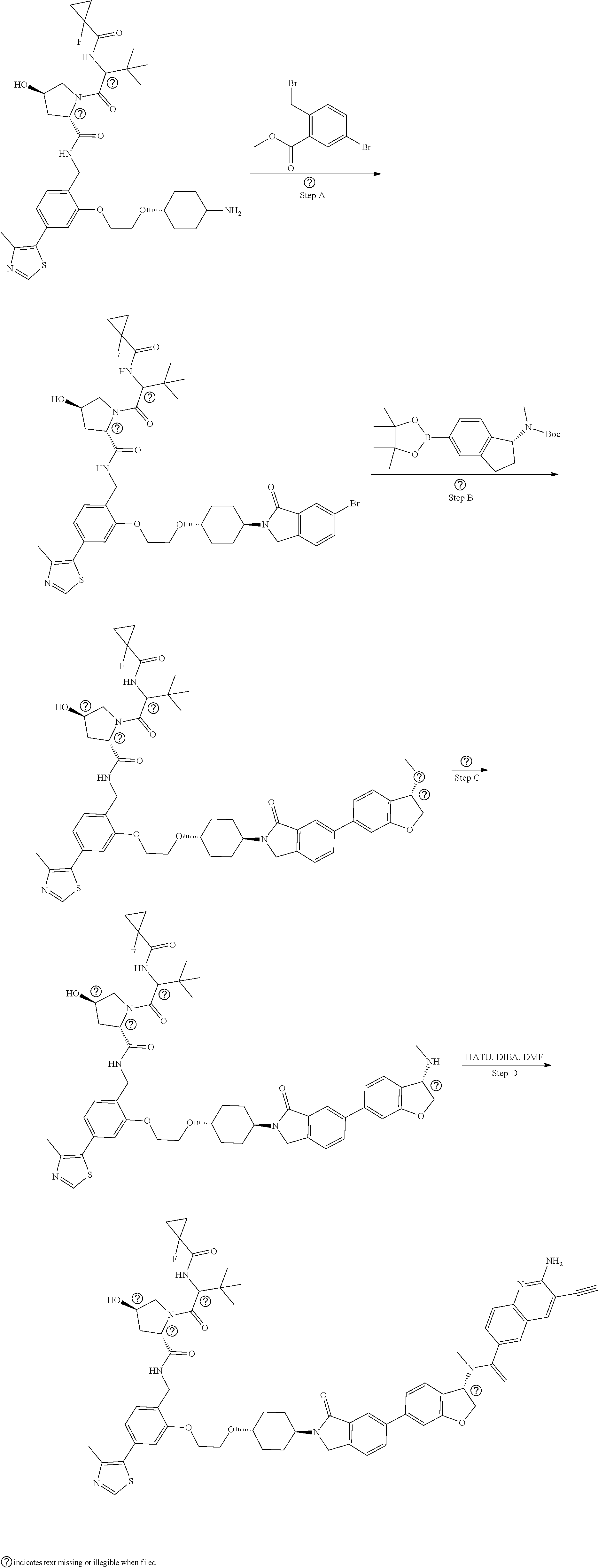

Example A316 was synthesized following the same procedures as described in Example A287 (7.6 mg, 11% yield over 4 steps) as a white solid. LC-MS (ESI): mass calcd. for C₆₃H₆₇FN8O9S, 1130.5; m/z found, 1131.5 [M+H]⁺. ¹H NMR (400 MHz, DMSO-d₆) δ 8.99 (s, 1H), 8.67 (s, 1H), 8.56-8.35 (m, 2H), 8.07 (s, 1H), 7.98-7.82 (m, 3H), 7.75 (s, 1H), 7.66 (d, J=7.8 Hz, 1H), 7.50 (d, J=7.8 Hz, 1H), 7.41 (d, J=7.8 Hz, 1H), 7.34 (d, J=6.3 Hz, 1H), 7.31-7.22 (m, 2H), 7.06 (s, 1H), 6.98 (d, J=9.1 Hz, 1H), 4.95 (s, 1H), 4.71-4.64 (m, 2H), 4.59 (d, J=9.3 Hz, 1H), 4.55-4.50 (m, 1H), 4.47 (s, 2H), 4.36 (s, 2H), 4.31-4.25 (m, 2H), 4.20-4.17 (m, 2H), 4.12-3.98 (m, 31), 3.86-3.79 (m, 2H), 3.69-3.58 (m, 2H), 3.46-3.38 (m, 1H), 2.67 (s, 3H), 2.47 (s, 4H), 2.16-2.10 (m, 3H), 1.98-1.89 (m, 1H), 1.84-1.75 (m, 2H), 1.73-1.62 (m, 2H), 1.41-1.32 (m, 4H), 1.26-1.19 (m, 2H), 0.95 (s, 9H).

### Example A318. 2-amino-3-ethynyl-N-((3S)-6-(6-(3-((2′-(((2S,4R)-1-((S)-2-(1-fluorocyclopropane-1-carboxamido)-3,3-dimethylbutanoyl)-4-hydroxypyrrolidine-2-carboxamido)methyl)-5′-(4-methylthiazol-5-yl)-2,3,4,5-tetrahydro-[1,1′-biphenyl]-4-yl)methyl)-3,8-diazabicyclo[3.2.1]octane-8-carbonyl)pyridin-3-yl)-2,3-dihydrobenzofuran-3-yl)-N-methylquinoline-6-carboxamide

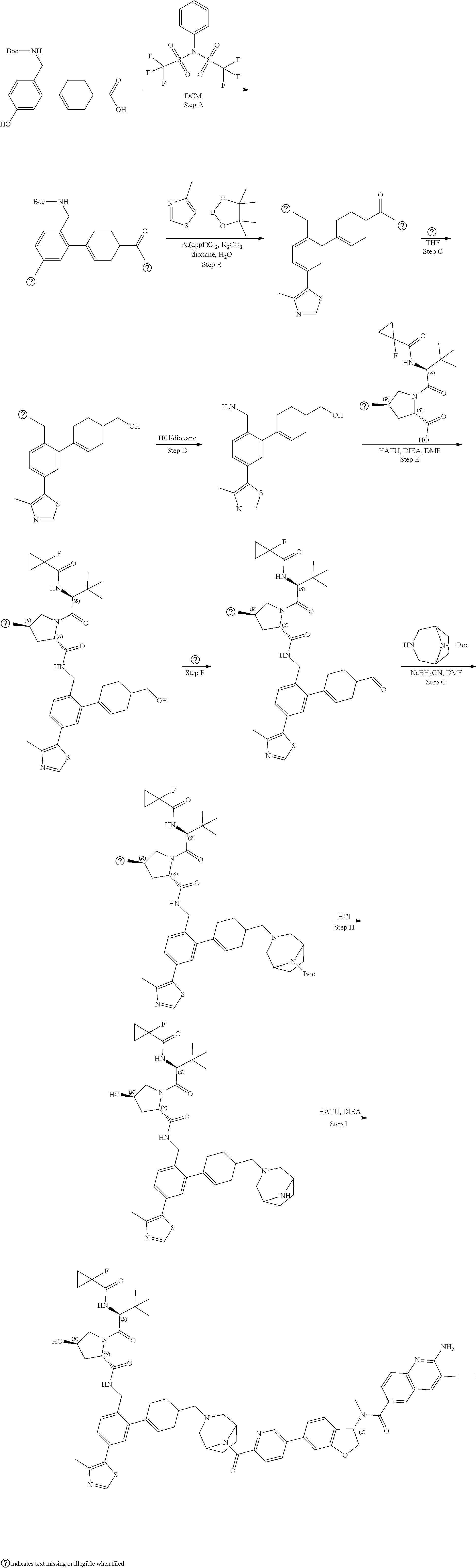

Example A318 was synthesized following the same procedures as described in Example A145 (5.6 mg, 2% yield over 9 steps) as a white solid. LC-MS (ESI): mass calcd. for C₆₆H₇₁FN₁₀O₇S, 1166.52; m/z found, 1163.3 [M+H]⁺. ¹H NMR (400 MHz, DMSO-d₆) δ 8.98 (s, 1H), 8.93 (s, 1H), 8.46 (s, 1H), 8.30 (s, 1H), 8.23 (dd, J=8.2, 2.1 Hz, 1H), 7.88 (s, 1H), 7.81 (d, J=8.2 Hz, 1H), 7.66 (dd, J=8.6, 1.8 Hz, 1H), 7.56-7.51 (m, 2H), 7.47 (d, J=8.2 Hz, 1H), 7.40 (d, J=7.5 Hz, 1H), 7.34 (s, 1H), 7.31 (d, J=9.7 Hz, 1H), 7.26 (d, J=9.2 Hz, 1H), 7.14 (s, 1H), 6.72 (s, 2H), 5.60 (s, 1H), 5.17 (d, J=3.4 Hz, 1H), 4.67 (s, 4H), 4.62 (s, 1H), 4.59 (d, J=9.1 Hz, 1H), 4.47 (t, J=8.0 Hz, 1H), 4.34 (s, 1H), 4.31-4.24 (m, 2H), 3.64-3.57 (m, 4H), 3.33-3.29 (m, 4H), 2.87-2.79 (m, 1H), 2.77-2.71 (m, 1H), 2.68 (s, 3H), 2.45 (s, 3H), 2.37-2.24 (m, 5H), 2.09-2.04 (m, 1H), 2.01-1.97 (m, 1H), 1.93-1.85 (m, 6H), 1.40-1.36 (m, 1H), 1.35-1.31 (m, 1H), 0.98-0.93 (m, 9H).

### Example A319. N1-(6-amino-4-(3-hydroxypropyl)pyridin-3-yl)-N2-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-N2-methyloxalamide

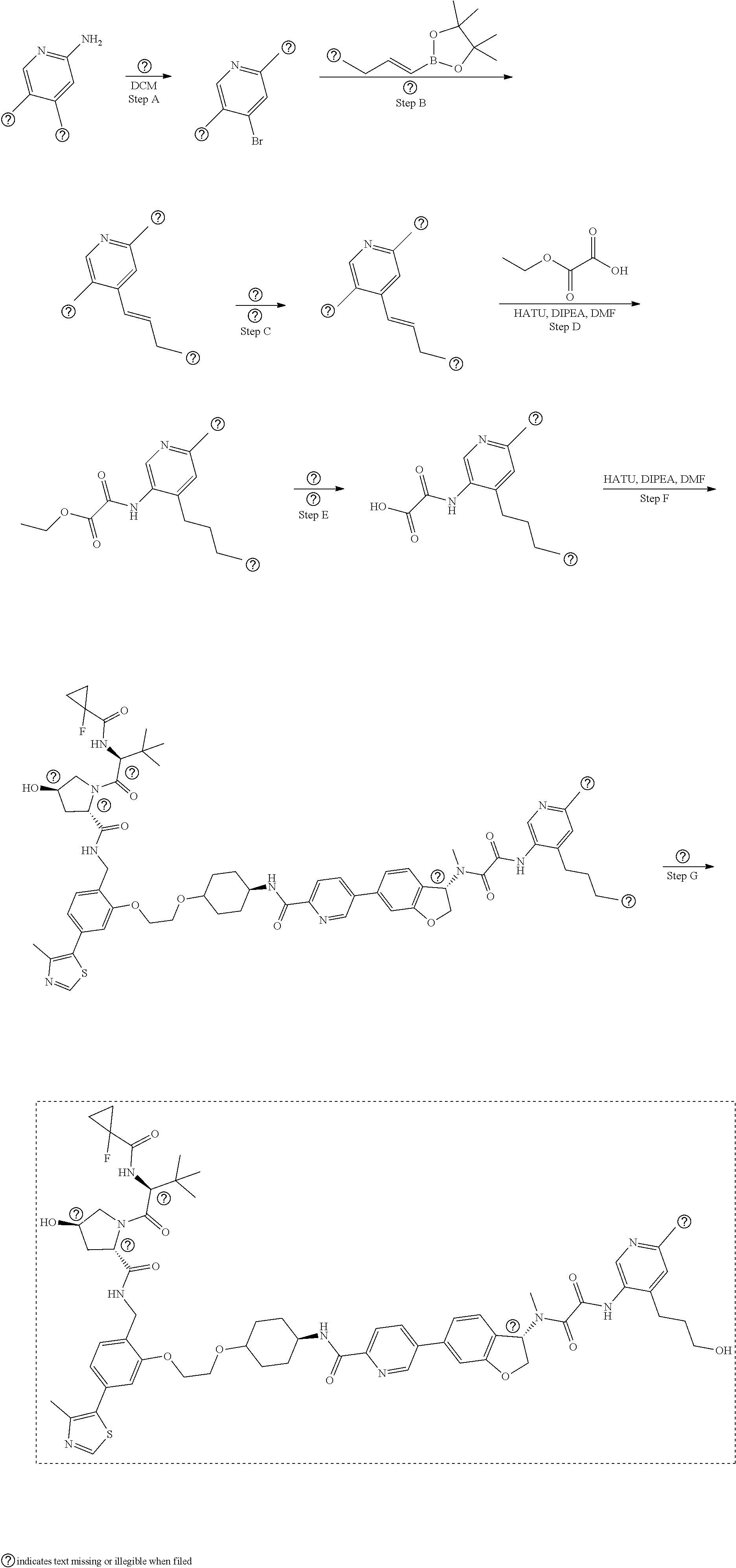

Step A: tert-butyl (4-bromo-5-nitropyridin-2-yl)(tert-butoxycarbonyl)carbamate

To a mixture of 4-bromo-5-nitropyridin-2-amine (1.0 g, 4.59 mmol, 1.0 eq.) in DCM (10 mL) was added DMAP (112 mg, 917 μmol, 0.2 eq.) and Di-tert-butyl dicarbonate (2.20 g, 10.1 mmol, 2.2 eq.) at 0° C. The reaction mixture was stirred at room temperature for 2 hours. The resulting solution was diluted with 50 mL of water and extracted with EtOAc (50 mL×3). The combined organic layers were washed with brine, dried over anhydrous Na₂SO₄ and concentrated under reduced pressure. The residue was purified by column chromatography (silica gel, 0% EtOAc in PE~10% EtOAc in PE) to give the tert-butyl (4-bromo-5-nitropyridin-2-yl)(tert-butoxycarbonyl)carbamate (1.6 g, 83% yield) as yellow solid. LC-MS (ESI): mass calcd. For C₁₅H₂₀BrN₃O₆, 417.05; m/z found, 418.3/420.3 [M+H]⁺.

Step B: tert-butyl (E)-(tert-butoxycarbonyl)(4-(3-((tert-butyldimethylsilyl)oxy) prop-1-en-1-yl)-5-nitropyridin-2-yl)carbamate

To a mixture of tert-butyl (4-bromo-5-nitropyridin-2-yl)(tert-butoxycarbonyl)carbamate (1.50 g, 3.59 mmol, 1.0 eq.) in 1,4-Dioxane (15 mL) and Water (1.5 mL) was added (E)-tert-butyldimethyl ((3-(4,4,5,5-tetramethyl-1,3,2-dioxaborolan-2-yl)allyl)oxy) silane (1.28 g, 4.30 mmol, 1.2 eq.), K₂CO₃ (1.49 g, 10.8 mmol, 3.0 eq.) and 1,1′-Bis(diphenylphosphino) ferrocene-palladium (II) dichloride (262 mg, 359 μmol, 0.1 eq.). The reaction mixture was stirred at 80° C. for 3 hours. The mixture was diluted with water (50 mL), extracted with EtOAc (50 mL×3). The combined organic layers dried over anhydrous Na₂SO₄, filtered and concentrated under reduced pressure to dryness. The residue was purified by flash column to give the tert-butyl (E)-(tert-butoxycarbonyl)(4-(3-((tert-butyldimethylsilyl)oxy) prop-1-en-1-yl)-5-nitropyridin-2-yl)carbamate (1.6 g, 87% yield) as yellow solid. LC-MS (ESI): mass calcd. For C₂₄H39N307Si, 509.26; m/z found, 510.4 [M+H]⁺.

Step C: tert-butyl (5-amino-4-(3-((tert-butyldimethylsilyl)oxy) propyl)pyridin-2-yl)(tert-butoxycarbonyl)carbamate

To a mixture of tert-butyl (E)-(tert-butoxycarbonyl)(4-(3-((tert-butyldimethylsilyl)oxy) prop-1-en-1-yl)-5-nitropyridin-2-yl)carbamate (400 mg, 785 μmol, 1.0 eq.) in MeOH (12.0 mL) was added palladium (40.0 mg, 376 μmol, 0.5 eq.). The mixture was stirred at room temperature for 16 hours under H₂. The mixture was purified by flash chromatography to afford tert-butyl (5-amino-4-(3-((tert-butyldimethylsilyl)oxy) propyl)pyridin-2-yl)(tert-butoxycarbonyl)carbamate (350 mg, 92% yield) as a white solid. LC-MS (ESI): mass calcd. For C₂₄H₄₃N305Si, 481.30; m/z found, 482.5 [M+H]⁺.

Steps D-G: N1-(6-amino-4-(3-hydroxypropyl)pyridin-3-yl)-N2-((S)-6-(6-(((1S,4S)-4-(2-(2-(((2S, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-N2-methyloxalamide

Example A319 was synthesized following the same procedures as described in Example A44 (2.2 mg, 1% yield over 4 steps) as a white solid. LC-MS (ESI): mass calcd. for C₅₉H₇₁FN₁₀O₁₁S, 1146.50; m/z found, 1147.8 [M+H]⁺. ¹H NMR (400 MHz, DMSO-d₆) δ 10.62-10.46 (m, 1H), 8.99 (s, 1H), 8.93 (d,)=2.0 Hz, 1H), 8.54-8.43 (m, 2H), 8.28 (dd, J=8.2, 2.1 Hz, 1H), 8.18-8.10 (m, 1H), 8.07 (d, J=6.0 Hz, 1H), 7.99-7.88 (m, 1H), 7.57-7.44 (m, 1H), 7.43-7.36 (m, 3H), 7.29 (dd, J=9.2, 2.4 Hz, 1H), 7.05 (d, J=1.1 Hz, 1H), 6.97 (dd, J=7.8, 1.1 Hz, 1H), 6.87 (d, J=2.6 Hz, 1H), 4.87-4.73 (m, 1H), 4.73-4.61 (m, 2H), 4.59 (d, J=9.2 Hz, 1H), 4.52 (t, J=8.2 Hz, 1H), 4.35 (d, J=4.2 Hz, 1H), 4.32-4.23 (m, 2H), 4.22-4.12 (m, 3H), 3.82-3.79 (m, 3H), 3.45-3.43 (m, 2H), 3.40-3.33 (m, 2H), 2.78 (s, 1H), 2.72-2.65 (m, 2H), 2.62 (s, 2H), 2.47 (s, 3H), 2.14-2.08 (m, 1H), 2.03 (s, 2H), 1.97-1.90 (m, 1H), 1.86 (d, J=10.8 Hz, 2H), 1.74-1.64 (m, 2H), 1.56-1.46 (m, 2H), 1.40-1.27 (m, 4H), 1.24-1.18 (m, 2H), 0.95 (s, 9H).

### Example A321. 2-amino-3-(azetidin-1-yl)-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

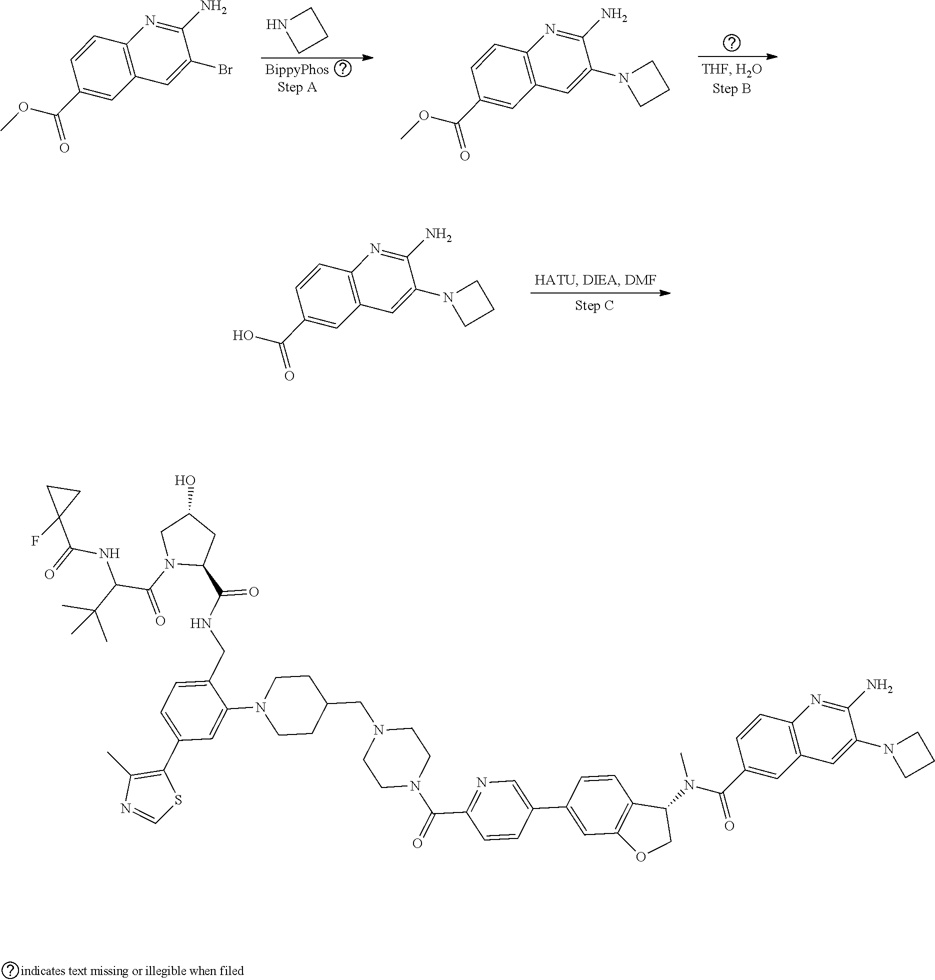

Step A: methyl 2-amino-3-(azetidin-1-yl)quinoline-6-carboxylate

To a mixture of methyl 2-amino-3-bromoquinoline-6-carboxylate (50 mg, 178 μmol, 1.0 eq.), Pd₂(dba)₃ (24 mg, 26.7 μmol, 0.15 eq.), xantphos (31 mg, 53.4 μmol, 0.3 eq.) and Cs₂CO₃ (174 mg, 534 μmol, 3.0 eq.) in 1,4-Dioxane (3 mL) was added azetidine (81 mg, 1.42 mmol, 8.0 eq.). The mixture was stirred at 110° C. for 2 hours under N₂ atmosphere. The resulting solution was diluted with 20 mL of water and extracted with EtOAc (30 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford methyl 2-amino-3-(azetidin-1-yl)quinoline-6-carboxylate (20.0 mg, 44% yield) as a yellow oil. LC-MS (ESI): mass calcd. For C₁₄H₁₅N₃O₂, 257.12; m/z found, 258.3 [M+H]⁺

Step B: 2-amino-3-(azetidin-1-yl)quinoline-6-carboxylic acid

To a mixture of methyl 2-amino-3-(azetidin-1-yl)quinoline-6-carboxylate (30 mg, 117 μmol, 1.0 eq.) in THF (2 mL) and H₂O (2 mL) was add LiOH (8 mg, 350 μmol, 3.0 eq.). The mixture was stirred at 50° C. for 12 hours. The resulting solution was concentrated under reduced pressure to afford 2-amino-3-(azetidin-1-yl)quinoline-6-carboxylic acid (23 mg, 81% yield) as a yellow oil, which was used directly without further purification. LC-MS (ESI): mass calcd. For C₁₃H₁₃N₃O₂, 243.10; m/z found, 244.2 [M+H]⁺.

Step C: 2-amino-3-(azetidin-1-yl)-N-(S)-6-16-(4-((1-(2-(((28,4R)-1-(S)-2-(1-fluorocyclopropane-1-carboxamido)-3, 3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

To a mixture of (2S,4R)-1-((S)-2-(1-fluorocyclopropane-1-carboxamido)-3,3-dimethylbutanoyl)-4-hydroxy-N-(2-(4-((4-(5-((S)-3-(methylamino)-2,3-dihydrobenzofuran-6-yl)picolinoyl)piperazin-1-yl)methyl)piperidin-1-yl)-4-(4-methylthiazol-5-yl)benzyl)pyrrolidine-2-carboxamide (30 mg, 31.6 μmol, 1.0 eq.), HATU (15 mg, 41.0 μmol, 1.3 eq.), 2-amino-3-(azetidin-1-yl)quinoline-6-carboxylic acid (8 mg, 34.7 μmol, 1.1 eq.) in DMF (3 mL) was added DIEA (12 mg, 94.7 μmol, 3.0 eq.). The mixture was stirred at room temperature for 2 hours under N2 atmosphere. The resulting solution was diluted with 10 mL of water and extracted with EtOAc. The combined organic layers were washed with brine, dried over anhydrous Na₂SO₄ and concentrated under reduced pressure. The mixture was purified by prep-HPLC (C18, 20~95%, MeCN in H₂O with 0.1% HCOOH) to afford 2-amino-3-(azetidin-1-yl)-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide (3.4 mg, 9% yield) as a yellow oil. LC-MS (ESI): mass calcd. For C₆₄H₇₅FN₁₂O₇S, 1174.56; m/z found, 1175.3 [M+H]⁺. ¹H NMR (400 MHz, DMSO-d₆) δ 9.57 (s, 1H), 8.99 (s, 1H), 8.93 (s, 1H), 8.51 (t, J=6.3 Hz, 1H), 8.46-8.35 (m, 1H), 8.28 (d, J=8.4 Hz, 1H), 7.98 (s, 1H), 7.78 (d, J=8.1 Hz, 1H), 7.68 (s, 2H), 7.60-7.53 (m, 2H), 7.44 (d, J=8.4 Hz, 1H), 7.41-7.32 (m, 2H), 7.29-7.24 (m, 1H), 7.15-7.12 (m, 2H), 4.88-4.66 (m, 2H), 4.60 (d, J=9.3 Hz, 2H), 4.49 (t, J=8.3 Hz, 1H), 4.39-4.32 (m, 3H), 4.01 (t, J=7.1 Hz, 4H), 3.70-3.61 (m, 7H), 3.17 (s, 3H), 3.11 (d, J=9.8 Hz, 3H), 2.79-2.63 (m, 6H), 2.46 (s, 3H), 2.34-2.26 (m, 2H), 2.15-2.03 (m, 1H), 2.02-1.82 (m, 5H), 1.47-1.43 (m, 1H), 1.41-1.37 (m, 1H), 1.36-1.33 (m, 1H), 1.25-1.19 (m, 3H), 0.98 (s, 9H).

### Example A323. 2-amino-3-(tert-butyl)-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

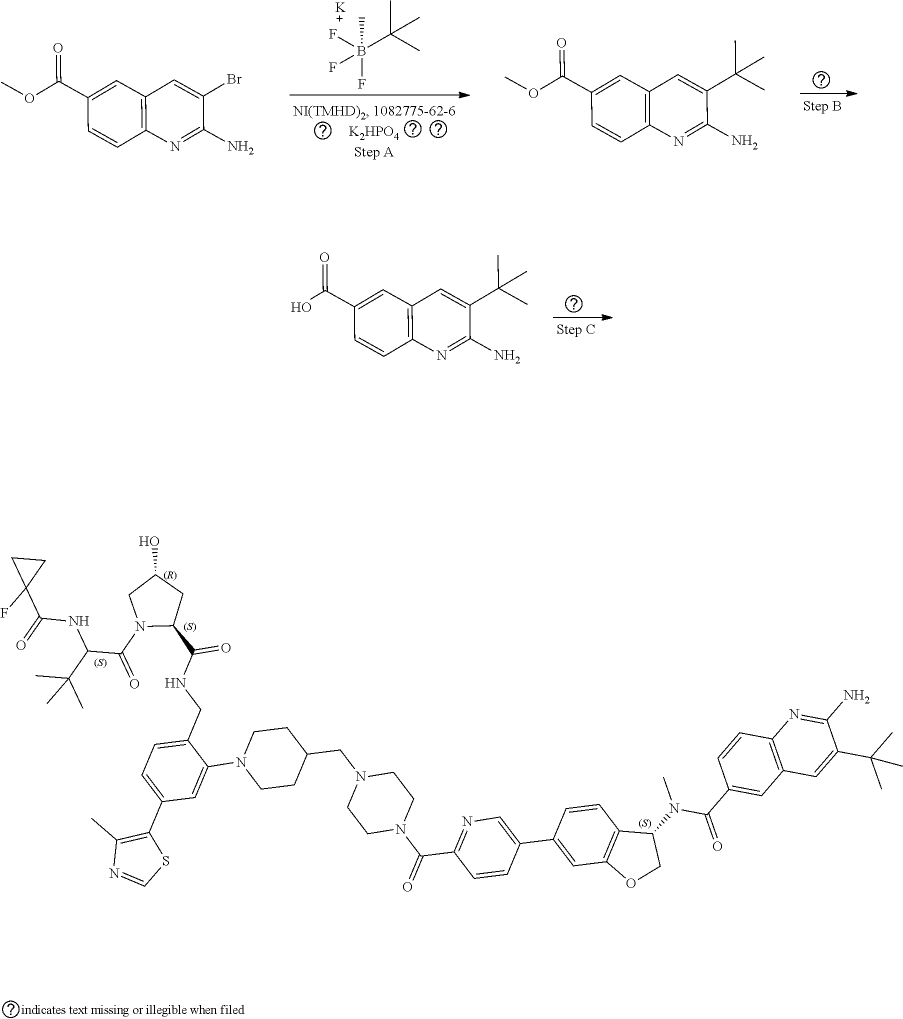

Step A: methyl 2-amino-3-(tert-butyl)quinoline-6-carboxylate

To a mixture of methyl 2-amino-3-bromoquinoline-6-carboxylate (500 mg, 1.78 mmol, 1.0 eq.), potassium tert-butyltrifluoroborate (583 mg, 3.56 mmol, 2.0 eq.), Zinc bromide (40 mg, 178 μmol, 0.1 eq.) and Nickel bis(dipivaloylmethanate)(76 mg, 178 μmol, 0.1 eq.) in DMA (40 mL) was added Potassium phosphate, dibasic (310 mg, 1.78 mmol, 1.0 eq.) and [Ir{dFCF₃ppy}₂(bpy)]PF₆ (18 mg, 17.8 μmol, 0.01 eq.). The mixture was stirred at room temperature (450 nm, 18 W) for 16 hours under N₂. The reaction mixture was poured into water (50 mL), and then extracted with EtOAc (50 mL×3). The combined organic layers were washed with brine, dried over anhydrous Na₂SO₄ and concentrated. The residue was purified by pre-HPLC to give methyl 2-amino-3-(tert-butyl)quinoline-6-carboxylate (10 mg, 2% yield) as a white solid.

LC-MS (ESI): mass calcd. For C₁₅H18N202, 258.14; m/z found, 259.2 [M+H]⁺.

Steps B-C: 2-amino-3-(tert-butyl)-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

Example A323 was synthesized following the same procedures as described in Example

A291 (0.7 mg, 1% yield over 3 steps) as a white solid. LC-MS (ESI): mass calcd. for C₆₅H78FN₁₁O₇S, 1175.58; m/z found, 1176.9 [M+H]⁺. ¹HNMR (400 MHz, DMSO-d₆) δ 8.99 (s, 1H), 8.93 (d, J=1.5 Hz, 1H), 8.51 (t, J=6.3 Hz, 1H), 8.40 (s, 1H), 8.28 (d, J=7.1 Hz, 1H), 7.89 (d, J=9.6 Hz, 1H), 7.78 (d, J=7.8 Hz, 2H), 7.60 (d, J=7.2 Hz, 1H), 7.46-7.39 (m, 2H), 7.35 (s, 1H), 7.28 (d, J=8.6 Hz, 1H), 7.17-7.09 (m, 3H), 4.83-4.72 (m, 2H), 4.60 (d, J=9.2 Hz, 2H), 4.49 (t, J=6.8 Hz, 1H), 4.39-4.34 (m, 3H), 3.66-3.62 (m, 5H), 3.18-3.12 (m, 6H), 2.72-2.65 (m, 8H), 2.46 (s, 3H), 2.07 (t, J=3.3 Hz, 1H), 1.95-1.85 (m, 4H), 1.56 (s, 9H), 1.48-1.43 (m, 2H), 1.37 (dd, J=10.3, 5.2 Hz, 2H), 1.24-1.21 (m, 2H), 0.98 (s, 9H).

### Example A324. N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)-N,1,3-trimethyl-1H-pyrazolo[4,3-c]quinoline-8-carboxamide

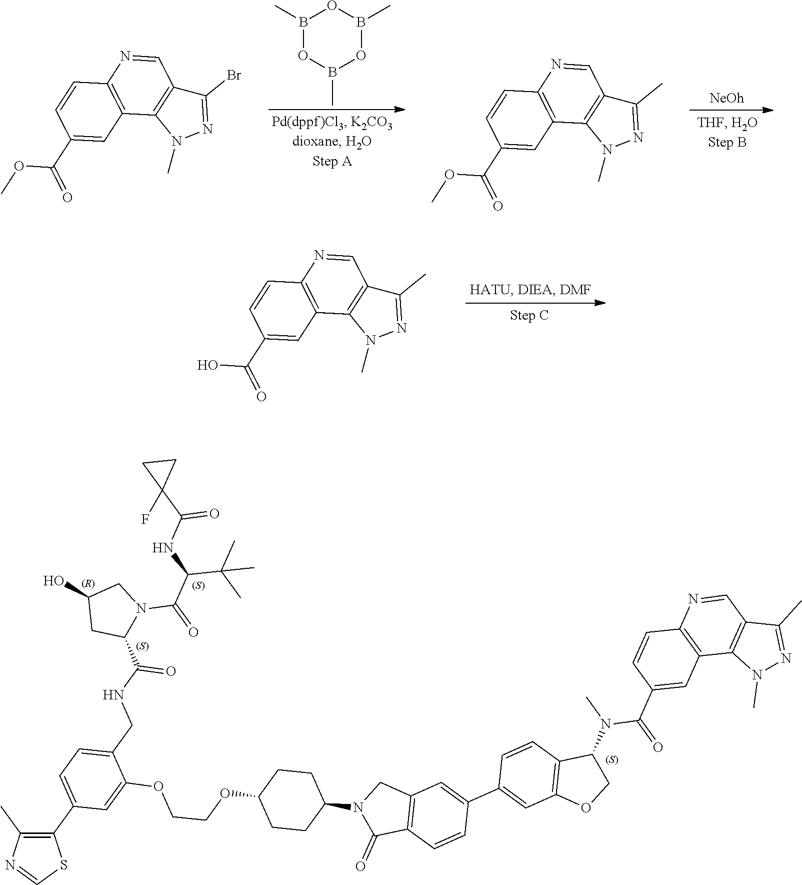

Example A324 was synthesized following the same procedures as described in Intermediate 3 and Example A287 (1 mg, 1% yield over 3 steps) as a white solid. LC-MS (ESI): mass calcd, for C₆₄H₇₀FN₉O₉S, 1159.5/z found, 1160.5 [M+H]⁺. ¹H NMR (400 MHz, DMSO-d₆) δ 9.45 (s, 1H), 8.99 (s, 1H), 8.75-8.57 (m, 1H), 8.54-8.45 (m, 1H), 8.28 (s, 1H), 8.05-7.94 (m, 1H), 7.86 (s, 1H), 7.80-7.71 (m, 2H), 7.64-7.53 (m, 1H), 7.41 (d, J=7.8 Hz, 1H), 7.36-7.31 (m, 1H), 7.30-7.19 (m, 2H), 7.06 (s, 1H), 6.98 (d, J=7.6 Hz, 1H), 4.82-4.74 (m, 1H), 4.59 (d, J=9.4 Hz, 1H), 4.53-4.44 (m, 5H), 4.38-4.23 (m, 3H), 4.22-4.14 (m, 2H), 4.09-4.00 (m, 1H), 3.85-3.79 (m, 2H), 3.66-3.58 (m, 4H), 2.77-2.73 (m, 2H), 2.65 (s, 3H), 2.53 (s, 3H), 2.47 (s, 3H), 2.19-2.07 (m, 3H), 2.01-1.88 (m, 1H), 1.84-1.76 (m, 1H), 1.73-1.62 (m, 1H), 1.45-1.29 (m, 4H), 1.25-1.18 (m, 4H), 0.95 (s, 9H).

### Example A325. N1-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N1-methyl-N2-(1-methyl-1H-pyrazolo[4,3-c]pyridin-7-yl) oxalamide

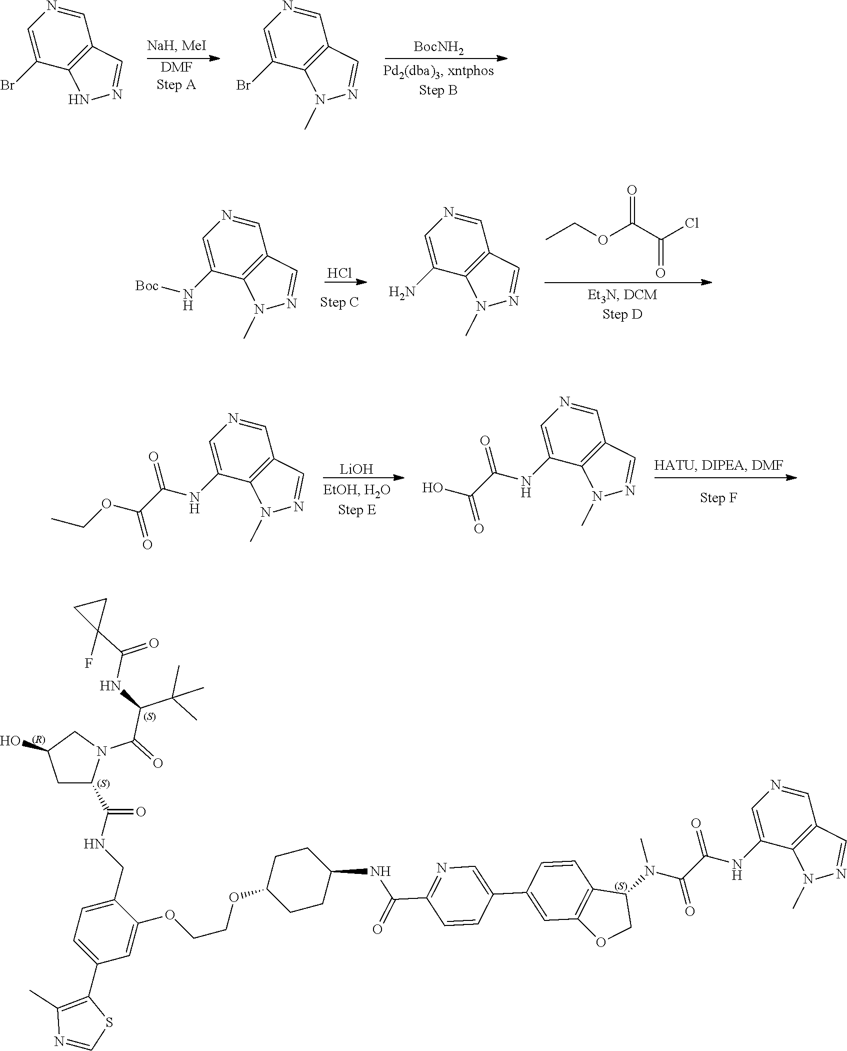

Step A: 7-bromo-1-methyl-1H-pyrazolo[4,3-c]pyridine

To a mixture of 7-bromo-1H-pyrazolo[4,3-c]pyridine (500 mg, 2.52 mmol, 1.0 eq.) in DMF (10 mL) was added NaH (79 mg, 3.28 mmol, 1.3 eq.) in batches at 0° C. and stirred for 30 minutes. Followed Mel (394 mg, 174 μL, 2.78 mmol, 1.1 eq.) was added dropwise. The mixture was stirred at room temperature for 1 hour, TLC showed the reaction was completed. The reaction mixture was quenched with NaHCO₃(aq. 50 mL), and then extracted with EtOAc (50 mL×2). The combined organic layers were washed with brine, dried over anhydrous Na₂SO₄ and concentrated. The residue was purified by column chromatography (silica gel, 0-3% of MeOH in DCM) to give 7-bromo-1-methyl-1H-pyrazolo[4,3-c]pyridine (200 mg, 37% yield) as a white solid. LC-MS (ESI): mass calcd. For CH₆BrN₃, 210.97/212.97; m/z found, 212.0/214.0 [M+H]⁺

Step B: tert-butyl (1-methyl-1H-pyrazolo[4,3-c]pyridin-7-yl)carbamate

To a mixture of 7-bromo-1-methyl-1H-pyrazolo[4,3-c]pyridine (100 mg, 472 μmol, 1.0 eq.), tert-butyl carbamate (166 mg, 1.41 mmol, 3.0 eq.), Pd₂(dba)₃ (44 mg, 47.2 μmol, 0.1 eq.) and xantphos (28 mg, 47.2 μmol, 0.1 eq.) in 1,4-Dioxane (5 mL) was added Cs₂CO₃ (230 mg, 707 μmol, 1.5 eq.). The mixture stirred at 100° C. for 4 hours under N₂. The mixture was concentrated under reduced pressure. The residue was purified by flash column chromatography on silica gel (silica gel, 0-6% MeOH in DCM) to give tert-butyl (1-methyl-1H-pyrazolo[4,3-c]pyridin-7-yl)carbamate (100 mg, 85% yield) as a yellow solid. LC-MS (ESI): mass calcd. For C₁₂H₁₆N₄O₂, 248.13; m/z found, 249.2 [M+H]⁺

Step C: 1-methyl-1H-pyrazolo[4,3-c]pyridin-7-amine

To a solution of tert-butyl (1-methyl-1H-pyrazolo[4,3-c]pyridin-7-yl)carbamate (100 mg, 403 μmol, 1.0 eq.) in DCM (2 mL) was added hydrochloric acid (1 mL, 4 M/L). The reaction was stirred at room temperature for 3 hours. The mixture was concentrated to afford 1-methyl-1H-pyrazolo[4,3-c]pyridin-7-amine (50 mg, 83% yield) as a yellow solid. LC-MS (ESI): mass calcd. For C₇H₈N₄, 148.07; m/z found, 149.2 [M+H]⁺

Step D: ethyl 2-((1-methyl-1H-pyrazolo[4,3-c]pyridin-7-yl)amino)-2-oxoacetate

To a mixture of 1-methyl-1H-pyrazolo[4,3-c]pyridin-7-amine (50 mg, 337 μmol, 1.0 eq.) and TEA (137 mg, 188 μL, 1.35 mmol, 4.0 eq.) in DCM (10 mL) was added ethyl 2-chloro-2-oxoacetate (55 mg, 405 μmol, 1.2 eq.) under N₂ at 0° C. The mixture was stirred at room temperature for 2 hours. The reaction mixture was poured into water (50 mL), extracted with DCM (50 mL×3). The combined organic layers were dried over anhydrous Na₂SO₄ and concentrated. The residue was purified by silica gel chromatography (silica gel, 0-6% of MeOH in DCM) to give ethyl 2-((1-methyl-1H-pyrazolo[4,3-c]pyridin-7-yl)amino)-2-oxoacetate (50 mg, 59% yield) as a yellow solid. LC-MS (ESI): mass calcd. For C₁₁H₁₂N₄O₃, 248.09; m/z found, 249.1 [M+H]⁺

Step E: 2-((1-methyl-1H-pyrazolo[4,3-c]pyridin-7-yl)amino)-2-oxoacetic acid

To a mixture of ethyl 2-((1-methyl-1H-pyrazolo[4,3-c]pyridin-7-yl)amino)-2-oxoacetate (50 mg, 201 μmol, 1.0 eq.) in EtOH (2 mL) and Water (2 mL) was added Lithium hydroxide monohydrate (10 mg, 6.72 μL, 242 μmol, 1.0 eq.) at 0° C. The mixture was stirred at room temperature for 1 hour to give yellow solution. The mixture was concentrated to afford 2-((1-methyl-1H-pyrazolo[4,3-c]pyridin-7-yl)amino)-2-oxoacetic acid (35 mg, 78% yield) as a yellow solid. LC-MS (ESI): mass calcd. For C₉H₈N₄O₃, 220.06; m/z found, 221.1 [M+H]⁺

Step F: N1-((S)-6-(6-(((15,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N1-methyl-N2-(1-methyl-III-pyrazolo[4,3-c]pyridin-7-yl) oxalamide

Example A325 was synthesized following the same procedures as described in Example A29 (3.0 mg, 4% yield) as a white solid. LC-MS (ESI): mass calcd, for C₅₈H₆₆FN1O10S, 1127.47; m/z found, 1128.8 [M+H]⁺. ¹H NMR (400 MHz, DMSO-d₆) δ 11.36-11.14 (m, 1H), 9.27 (d, J=11.1 Hz, 1H), 8.99 (s, 1H), 8.94 (s, 1H), 8.58 (d, J=8.9 Hz, 1H), 8.53-8.42 (m, 3H), 8.29 (d, J=8.2 Hz, 1H), 8.09 (d, J=8.3 Hz, 1H), 7.59-7.48 (m, 1H), 7.44-7.37 (m, 3H), 7.29 (dd, J=9.3, 2.5 Hz, 1H), 7.05 (s, 1H), 6.97 (d, J=7.7 Hz, 1H), 4.86-4.65 (m, 3H), 4.59 (d, J=9.1 Hz, 1H), 4.52 (t, J=8.3 Hz, 1H), 4.37-4.16 (m, 10H), 3.83-3.78 (m, 2H), 3.69-3.58 (m, 2H), 2.87 and 2.68 (sx2, 3H), 2.47 (s, 3H), 2.09-2.01 (m, 3H), 1.98-1.93 (m, 1H), 1.88-1.83 (m, 2H), 1.55-1.47 (m, 2H), 1.41-1.31 (m, 4H), 1.24-1.23 (m, 2H), 0.95 (s, 9H).

### Example A326. N-((S)-6-(6-(((18,4S)-4-(2-(2-(((2,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N,1,3-trimethyl-1H-pyrazolo[4,3-c]quinoline-8-carboxamide

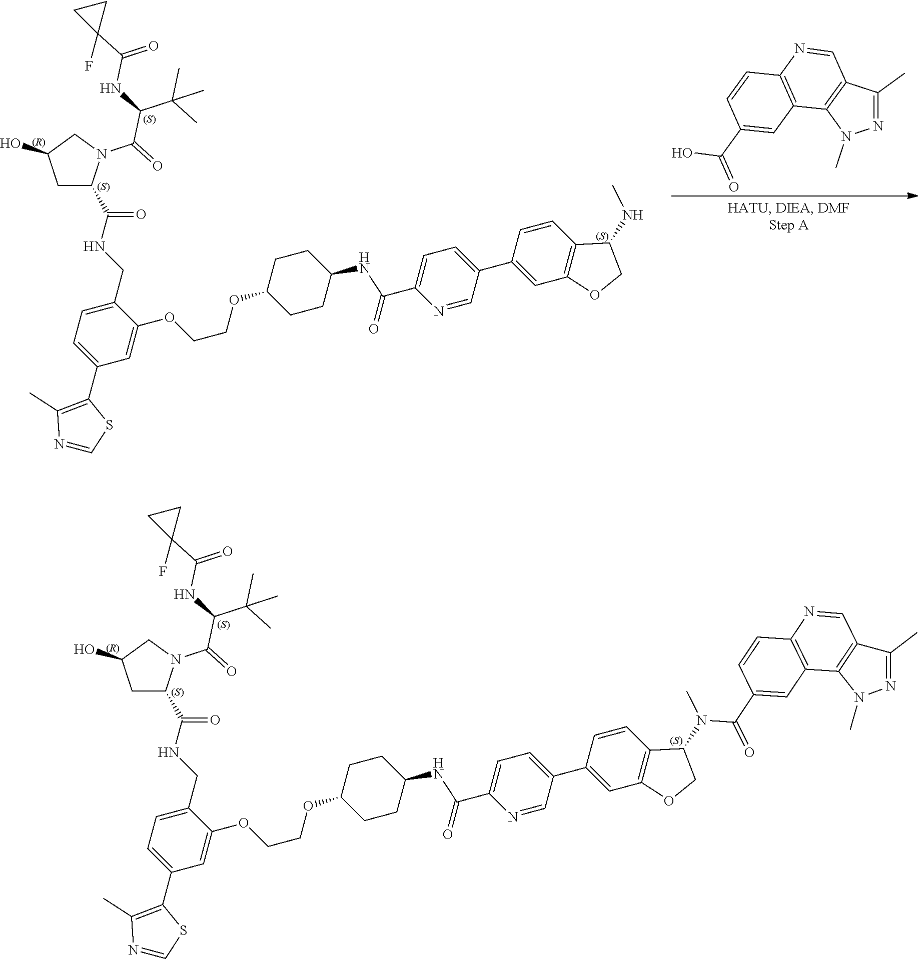

Example A326 was synthesized following the same procedures as described in Example A114 (1.5 mg, 1% yield) as a white solid. LC-MS (ESI): mass calcd, for C₆₂H₆₉FN₁₀O₉S, 1148.50; m/z found, 1149.9 [M+H]⁺. ¹HNMR (400 MHz, DMSO-d₆) δ 9.68 (s, 1H), 8.99 (s, 1H), 8.93 (s, 1H), 8.71 (s, 1H), 8.53-8.44 (m, 2H), 8.37-8.24 (m, 2H), 8.16-8.04 (m, 2H), 7.64 (s, 1H), 7.44-7.27 (m, 4H), 7.05 (s, 1H), 6.97 (d, J=7.7 Hz, 1H), 4.79 (s, 1H), 4.59 (d, J=9.6 Hz, 1H), 4.55 (s, 2H), 4.51 (d, J=7.9 Hz, 1H), 4.36-4.28 (m, 3H), 4.25-4.16 (m, 4H), 3.82-3.79 (m, 4H), 2.73-2.65 (m, 6H), 2.47 (s, 3H), 2.11-2.01 (m, 3H), 1.96-1.92 (m, 1H), 1.88-1.83 (m, 2H), 1.59-1.45 (m, 2H), 1.42-1.28 (m, 4H), 1.24-1.17 (m, 2H), 0.95 (s, 9H).

### Example A327. N1-(4-amino-2-ethyl-2,3-dihydro-1H-pyrrolo[3,4-c]pyridin-7-yl)-N2-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N2-methyloxalamide

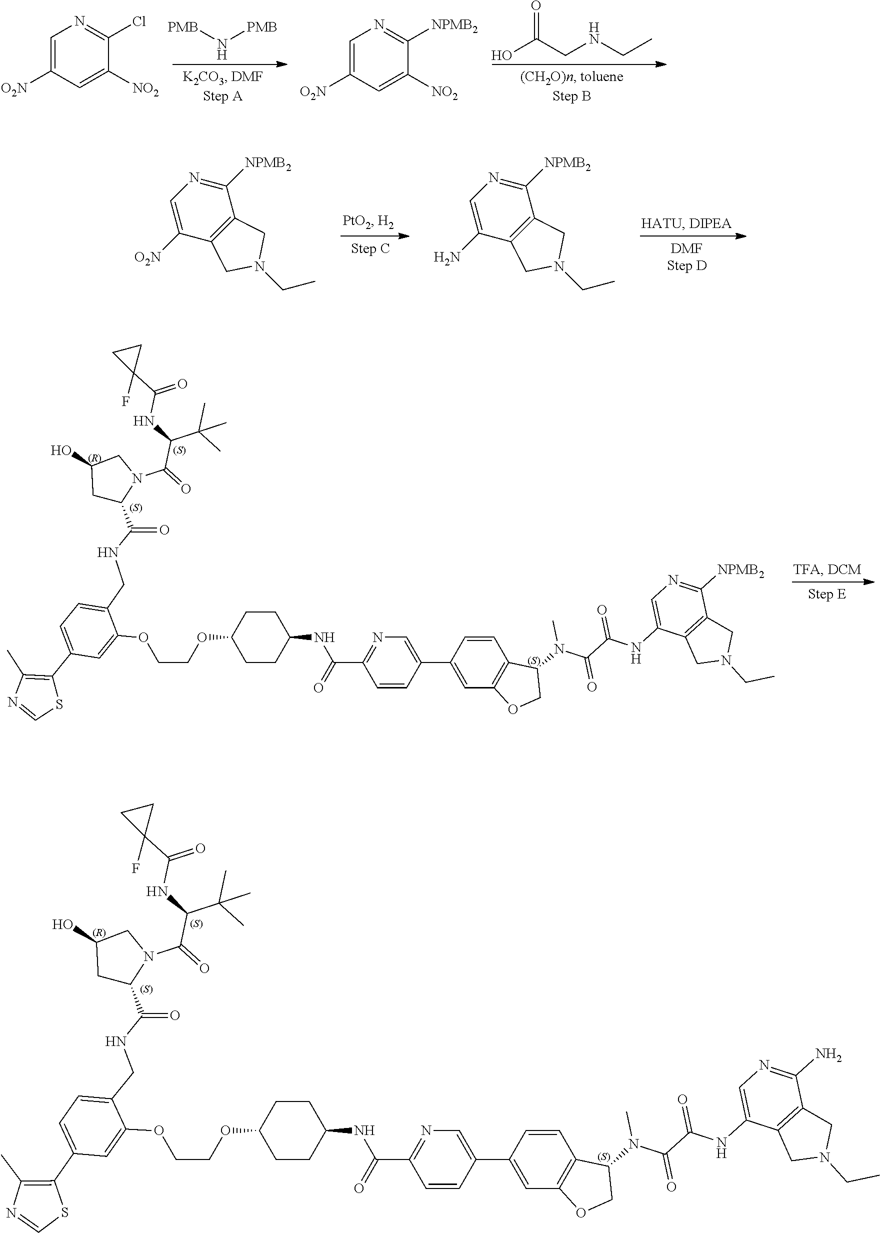

Step A: N,N-bis(4-methoxybenzyl)-3,5-dinitropyridin-2-amine

To a solution of 2-chloro-3,5-dinitropyridine (3 g, 14.7 mmol, 1.0 eq.) in DMF (30 mL) was added bis(4-methoxybenzyl) amine (5.69 g, 22.1 mmol, 1.5 eq.) and K₂CO₃ (5.09 g, 36.8 mmol, 2.5 eq.) at room temperature. The mixture was then stirred at room temperature for 16 hours under N2 atmosphere. The resulting solution was diluted with 30 mL of water and extracted with EtOAc (30 mL×3). The combined organic layers were washed with brine, dried over anhydrous Na₂SO₄ and concentrated under reduced pressure. The residue was purified by column chromatography (silica gel, 0% EtOAc in PE~15% EtOAc in PE) to afford N,N-bis(4-methoxybenzyl)-3,5-dinitropyridin-2-amine (5.9 g, 94% yield) as a yellow oil. LC-MS (ESI): mass calcd. For C₂₁H₂₀N₄O₆, 424.1; m/z found, 425.1 [M+H]⁺.

Step B: 2-ethyl-N,N-bis(4-methoxybenzyl)-7-nitro-2,3-dihydro-1H-pyrrolo[3,4-c]pyridin-4-amine

To a solution of N,N-bis(4-methoxybenzyl)-3,5-dinitropyridin-2-amine (1 g, 2.36 mmol, 1.0 eq.) in Toluene (10 mL) was added ethylglycine (1.46 g, 14.1 mmol, 6.0 eq.) and Paraformaldehyde (425 mg, 14.1 mmol, 6.0 eq.) at room temperature. The mixture was then stirred at 110° C. for 3 hours under N₂ atmosphere. The resulting solution was diluted with 30 mL of water and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~25% EtOAc in PE) to afford 2-ethyl-N,N-bis(4-methoxybenzyl)-7-nitro-2,3-dihydro-1H-pyrrolo[3,4-c]pyridin-4-amine (70 mg, 7% yield) as a yellow oil. LC-MS (ESI): mass calcd. For C₂₅H₂₈N₄O₄, 448.2; m/z found, 449.4 [M+H]⁺.

Step C: 2-ethyl-N4,N4-bis(4-methoxybenzyl)-2,3-dihydro-1H-pyrrolo[3,4-c]pyridine-4,7-diamine

To a solution of 2-ethyl-N,N-bis(4-methoxybenzyl)-7-nitro-2,3-dihydro-1H-pyrrolo[3,4-c]pyridin-4-amine (70 mg, 156 μmol, 1.0 eq.) in MeOH (5 mL) was added PtO₂/C (14 mg, 72 μmol, 0.5 eq.) at room temperature. The mixture was then stirred at room temperature for 16 hours under H₂ atmosphere. The mixture was filtered and the resulting solution was concentrated under reduced pressure. The residue was purified by column chromatography (silica gel, 0% MeOH in DCM~10% MeOH in DCM) to afford 2-ethyl-N4,N4-bis(4-methoxybenzyl)-2,3-dihydro-1H-pyrrolo[3,4-c]pyridine-4,7-diamine (15 mg, 23% yield) as a yellow oil. LC-MS (ESI): mass calcd. For C₂₅H₃₀N₄O₂, 418.2; m/z found, 419.2 [M+H]⁺.

Steps D-E: N1-(4-amino-2-ethyl-2,3-dihydro-1H-pyrrolo[3,4-c]pyridin-7-yl)-N2-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N2-methyloxalamide

Example A327 was synthesized following the same procedures as described in Example A31 (2 mg, 3% yield over 5 steps) as a white solid. LC-MS (ESI): mass calcd, for C₆₀H₇₂FN11010S, 1157.52/z found, 1158.5 [M+H]⁺. ¹H NMR (400 MHz, DMSO-d₆) δ 10.88-10.69 (m, 1H), 8.99 (s, 1H), 8.93 (s, 1H), 8.56-8.40 (m, 2H), 8.28 (d, J=8.3 Hz, 1H), 8.13-7.96 (m, 2H), 7.60-7.44 (m, 1H), 7.41 (d, J=7.5 Hz, 2H), 7.38 (s, 1H), 7.29 (d, J=6.7 Hz, 1H), 7.05 (s, 1H), 6.97 (d, J=7.8 Hz, 1H), 4.82-4.75 (m, 1H), 4.74-4.68 (m, 1H), 4.67-4.63 (m, 1H), 4.61-4.58 (m, 1H), 4.54-4.50 (m, 1H), 4.39-4.32 (m, 2H), 4.31-4.28 (m, 1H), 4.27-4.21 (m, 1H), 4.19-4.14 (m, 2H), 3.82-3.80 (m, 2H), 3.78-3.71 (m, 1H), 3.68-3.64 (m, 1H), 3.63-3.58 (m, 2H), 3.35-3.32 (m, 4H), 3.25-3.23 (m, 1H), 2.78-2.63 (m, 3H), 2.47 (s, 3H), 2.14-1,98 (m, 4H), 1.96-1.91 (m, 1H), 1.89-1.82 (m, 2H), 1.55-1.47 (m, 2H), 1.38-1.33 (m, 2H), 1.30-1.20 (m, 6H), 0.96 (s, 9H).

### Example A328. 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-4-(3-hydroxypropyl)-N-methylquinoline-6-carboxamide

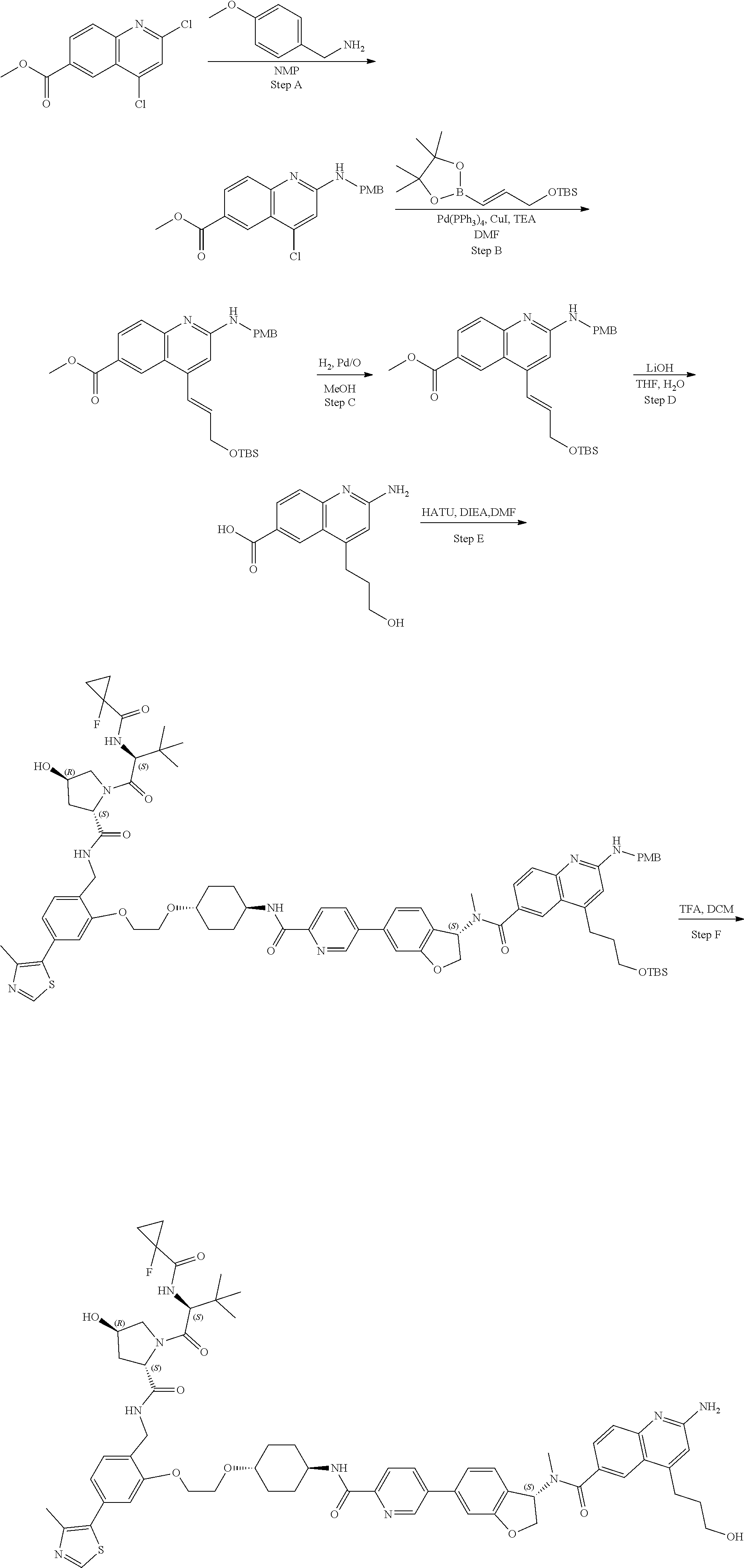

Step A: methyl 4-chloro-2-((4-methoxybenzyl)amino) quinoline-6-carboxylate

To a mixture of methyl 2,4-dichloroquinoline-6-carboxylate (500 mg, 1.95 mmol, 1.0 eq.) in NMP (15 mL) was added (4-methoxyphenyl)methanamine (536 mg, 3.91 mmol, 2.0 eq.). The mixture was stirred at 100° C. for 12 hours under N₂ atmosphere. The resulting solution was diluted with 10 mL of water and extracted with EtOAc (20 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford methyl 4-chloro-2-((4-methoxybenzyl)amino) quinoline-6-carboxylate (300 mg, 43% yield) as a yellow oil. LC-MS (ESI): mass calcd. For C₁₉H₁₇ClN₂O₃, 356.09; m/z found, 357.2 [M+H]⁺.

Step B: methyl (E)-4-(3-((tert-butyldimethylsilyl)oxy) prop-1-en-1-yl)-2-((4-methoxybenzyl)amino) quinoline-6-carboxylate

To a mixture of methyl 4-chloro-2-((4-methoxybenzyl)amino) quinoline-6-carboxylate (20.0 mg, 56.1 μmol, 1.0 eq.), 1,1′-Bis(di-t-butylphosphino) ferrocene palladium dichloride (4 mg, 5.61 μmol, 0.1 eq.), Cs₂CO₃ (55 mg, 168 μmol, 3.0 eq.) in 1,4-Dioxane (3 mL) and H₂O (0.3 mL) was added (E)-tert-butyldimethyl ((3-(4,4,5,5-tetramethyl-1,3,2-dioxaborolan-2-yl)allyl)oxy) silane (33.4 mg, 112 μmol, 2.0 eq.). The mixture was stirred at 100° C. for 2 hours under N₂ atmosphere. The resulting solution was diluted with 10 mL of water and extracted with EtO Ac (20 mL×3). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford methyl 4-(3-((tert-butyldimethylsilyl)oxy) prop-1-en-1-yl)-2-((4-methoxybenzyl)amino) quinoline-6-carboxylate (12.0 mg, 44% yield) as a yellow oil LC-MS (ESI): mass calcd. For C₂₈H36N₂O₄Si, 492.24; m/z found, 495.3 [M+H]⁺.

Step C: methyl 4-(3-((tert-butyldimethylsilyl)oxy) propyl)-2-((4-methoxybenzyl)amino) quinoline-6-carboxylate

To a mixture of methyl 4-(3-((tert-butyldimethylsilyl)oxy) prop-1-en-1-yl)-2-((4-methoxybenzyl)amino) quinoline-6-carboxylate (100 mg, 203 μmol, 1.0 eq.), Pd/C (6 mg, 60.9 μmol, 0.3 eq.), Pd (OH) 2 (8 mg, 60.9 μmol, 0.3 eq.) in MeOH (15 mL). The mixture was stirred at 50° C. for 12 hours under H₂ atmosphere. The combined organic layers were concentrated under reduced pressure to afford methyl 4-(3-((tert-butyldimethylsilyl)oxy) propyl)-2-((4-methoxybenzyl)amino) quinoline-6-carboxylate (50.0 mg, 50% yield) as a yellow oil. LC-MS (ESI): mass calcd. For C₂₈H₃₈N₂O₄Si, 494.26; m/z found, 495.3 [M+H]⁺.

Step D: 2-amino-4-(3-hydroxypropyl)quinoline-6-carboxylic acid

To a mixture of methyl 4-(3-((tert-butyldimethylsilyl)oxy) propyl)-2-((4-methoxybenzyl)amino) quinoline-6-carboxylate (50 mg, 101 μmol, 1.0 eq.) in THF (3 mL) and H₂O (1 mL) was added LiOH (7 mg, 303 μmol, 3.0 eq.). The mixture was stirred at room temperature for 12 hours under N₂ atmosphere. The combined organic layers were under reduced pressure to afford 4-(3-((tert-butyldimethylsilyl)oxy) propyl)-2-((4-methoxybenzyl)amino) quinoline-6-carboxylic acid (43.0 mg, 89% yield) as a yellow oil. LC-MS (ESI): mass calcd. For C₁₃H₁₄N₂O₃, 246.10; m/z found, 247.0 [M+H]⁺.

Steps E-F: 2-amino-N-(S)-6-(6-(((18,4S)-4-(2-(2-(2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-4-(3-hydroxypropyl)-N-methylquinoline-6-carboxamide

Example A328 was synthesized following the same procedures as described in Example A13 (2.7 mg, 20% yield over 2 steps) as a white solid. LC-MS (ESI): mass calcd. for C₆₂H₇₂FN₉O₁₀S, 1153.51; m/z found, 1154.5 [M+H]⁺. ¹H NMR (400 MHz, DMSO-d₆) δ 8.98 (s, 1H), 8.92 (s, 1H), 8.53-8.44 (m, 2H), 8.27 (dd, J=8.2, 2.2 Hz, 1H), 8.08 (d, J=8.3 Hz, 1H), 7.97 (s, 1H), 7.63-7.55 (m, 2H), 7.51 (d, J=8.6 Hz, 1H), 7.40 (t, J=8.9 Hz, 2H), 7.34-7.27 (m, 2H), 7.05 (s, 1H), 6.97 (d, J=9.1 Hz, 1H), 6.69 (s, 2H), 5.17 (d, J=3.6 Hz, 1H), 4.72 (s, 2H), 4.64 (t, J=5.1 Hz, 1H), 4.59 (d, J=8.7 Hz, 1H), 4.52 (t, J=8.2 Hz, 1H), 4.35 (s, 1H), 4.33-4.19 (m, 2H), 4.17 (s, 2H), 3.80 (d, J=4.1 Hz, 2H), 3.70-3.56 (m, 2H), 3.53 (dd, J=11.4, 6.0 Hz, 2H), 3.36 (s, 2H), 2.99-2.93 (m, 2H), 2.68 (s, 3H), 2.46 (s, 3H), 2.10-2.01 (m, 3H), 1.97-1.91 (m, 1H), 1.86-1.75 (m, 4H), 1.55-1.47 (m, 2H), 1.39-1.28 (m, 4H), 1.22-1.18 (m, 2H), 0.95 (s, 9H).

### Example A329. 3-ethyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)-N,1-dimethyl-1H-pyrazolo[4,3-c]quinoline-8-carboxamide

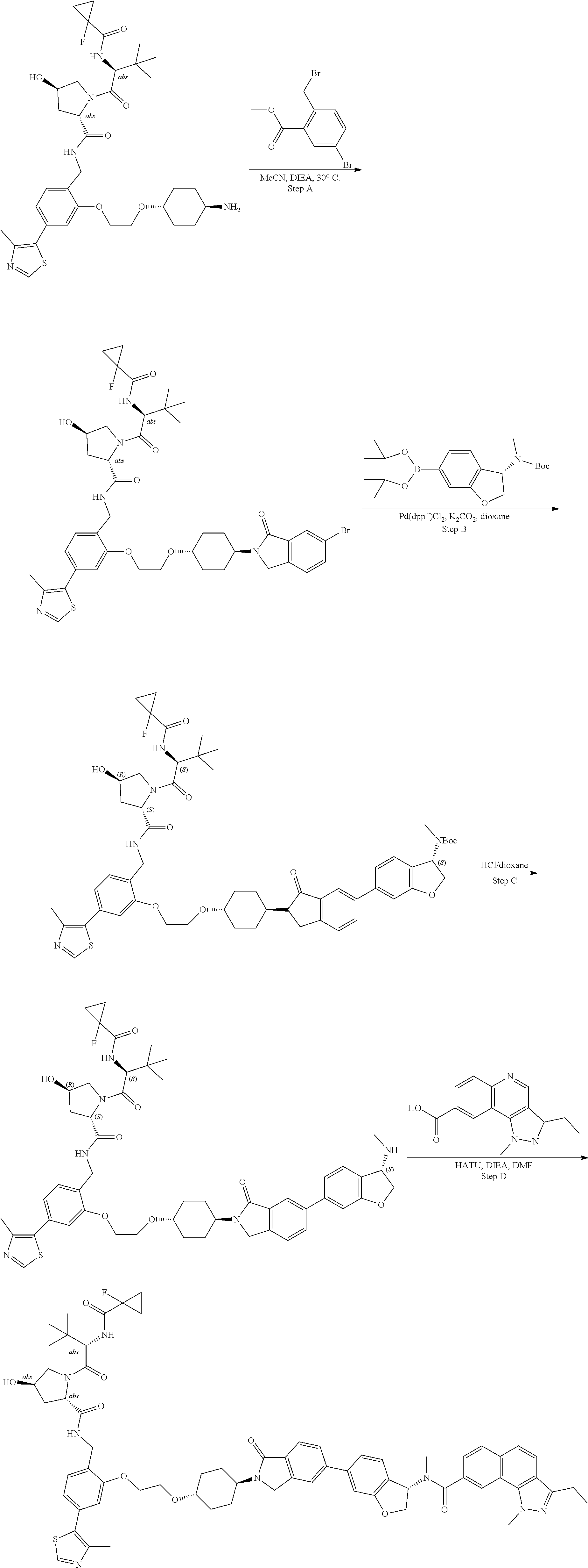

Step A: (2S,4R)-N-(2-(2-(((1r,4S)-4-(5-bromo-1-oxoisoindolin-2-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of (2S,4R)-N-(2-(2-(((1r,4S)-4-aminocyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (500 mg, 742 μmol, 1.0 eq.) and methyl 4-bromo-2-(bromomethyl)benzoate (343 mg, 1.11 mmol, 1.5 eq.) in MeCN (10 mL) was added DIEA (19 mg, 25.9 μL, 148 μmol, 5.0 eq.) at room temperature. The resulting mixture was stirred at 80° C. for 6 hours. The mixture was concentrated under reduced pressure. The residue was purified by flash column chromatography on silica gel (silica gel, 0-5% MeOH in DCM) to give (2S,4R)-N-(2-(2-(((1r,4S)-4-(5-bromo-1-oxoisoindolin-2-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (600 mg, 93% yield) as a yellow solid.

LC-MS (ESI): mass calcd. For C₄₂H₅₁BrFN₅O₇S, 867.27/869.27; m/z found, 868.1/870.1 [M+H]⁺

Step B: tert-butyl ((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)(methyl)carbamate

To a mixture of tert-butyl(S)-methyl (6-(3,3,4,4-tetramethylborolan-1-yl)-2,3-dihydrobenzofuran-3-yl)carbamate (154 mg, 414 μmol, 1.2 eq.), PdCl₂ (dppf)(25 mg, 34.5 μmol, 0.1 eq.), (2S,4R)-N-(2-(2-(((1r,4S)-4-(5-bromo-1-oxoisoindolin-2-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (300 mg, 345 μmol, 1.0 eq.) in 1,4-Dioxane (8 mL) and Water (0.5 mL) was added K₂CO₃ (95 mg, 691 μmol, 2.0 eq.). The reaction mixture was purged with nitrogen three times, then heated to 100° C. for 2 hours under nitrogen. After cooling to room temperature, the mixture was concentrated under reduced pressure, the residue was purified by silica gel chromatography (silica gel, 0-6% of MeOH in DCM) to give tert-butyl ((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)(methyl)carbamate (200 mg, 55% yield) as a yellow solid. LC-MS (ESI): mass calcd. For C₅₆H₆₉FN6010S, 1036.48; m/z found, 1037.4 [M+H]⁺

Step C: (2S, 4R)-1-((S)-2-(1-fluorocyclopropane-1-carboxamido)-3,3-dimethylbutanoyl)-4-hydroxy-N-(2-(2-(((1S,4S)-4-(5-((S)-3-(methylamino)-2,3-dihydrobenzofuran-6-yl)-1-oxoisoindolin-2-yl)cyclohexyl)oxy)ethoxy)-4-(4-methylthiazol-5-yl)benzyl)pyrrolidine-2-carboxamide

To a solution of tert-butyl ((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)(methyl)carbamate (200 mg, 193 μmol, 1.0 eq.) in DCM (2 mL) was added HCl-dioxane (1 mL). The reaction was stirred at room temperature for 1 hour. The mixture was concentrated to afford (2S,4R)-1-((S)-2-(1-fluorocyclopropane-1-carboxamido)-3,3-dimethylbutanoyl)-4-hydroxy-N-(2-(2-(((1S,4S)-4-(5-((S)-3-(methylamino)-2,3-dihydrobenzofuran-6-yl)-1-oxoisoindolin-2-yl)cyclohexyl)oxy)ethoxy)-4-(4-methylthiazol-5-yl)benzyl)pyrrolidine-2-carboxamide (180 mg, 99% yield) as a yellow solid. LC-MS (ESI): mass calcd. For C₅₁H₆₁FN₆O₈S, 936.43; m/z found, 937.8 [M+H]⁺

Step D: 3-ethyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxoisoindolin-5-yl)-2,3-dihydrobenzofuran-3-yl)-N, 1-dimethyl-1H-pyrazolo[4,3-c]quinoline-8-carboxamide

Example A329 was synthesized following the same procedures as described in Example A287 (2 mg, 8% yield) as a yellow solid. LC-MS (ESI): mass calcd, for C₆₅H₇₂FNO₉S, 1173; m/z found, 587.6 [M/2+H]⁺. ¹H NMR (400 MHz, DMSO-d₆) δ 9.69 (s, 1H), 8.99 (s, 1H), 8.93 (s, 1H), 8.72 (d, J=4.9 Hz, 1H), 8.50 (t, J=6.1 Hz, 2H), 8.38-8.24 (m, 2H), 8.08 (d, J=8.0 Hz, 2H), 7.69-7.60 (m, 1H), 7.46-7.39 (m, 2H), 7.34-7.27 (m, 2H), 7.05 (s, 1H), 6.97 (d, J=7.8 Hz, 1H), 4.89-4.76 (m, 2H), 4.73-4.64 (m, 1H), 4.61-4.50 (m, 6H), 4.36 (s, 1H), 4.30 (d, J=6.0 Hz, 1H), 4.25 (d, J=5.7 Hz, 1H), 4.19-4.16 (m, 2H), 3.82-3.79 (m, 3H), 3.68-3.61 (m, 2H), 3.13 (q, J=7.5 Hz, 4H), 2.77-2.67 (m, 3H), 2.47 (s, 3H), 2.11-2.02 (m, 3H), 1.98-1.92 (m, 1H), 1.89-1.83 (m, 2H), 1.56-1.47 (m, 2H), 1.40 (t, J=7.6 Hz, 3H), 1.36-1.29 (m, 3H), 1.25-1.19 (m, 3H), 0.96 (s, 9H).

### Example A330. 3-ethyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,1-dimethyl-1H-pyrazolo[4,3-c]quinoline-8-carboxamide

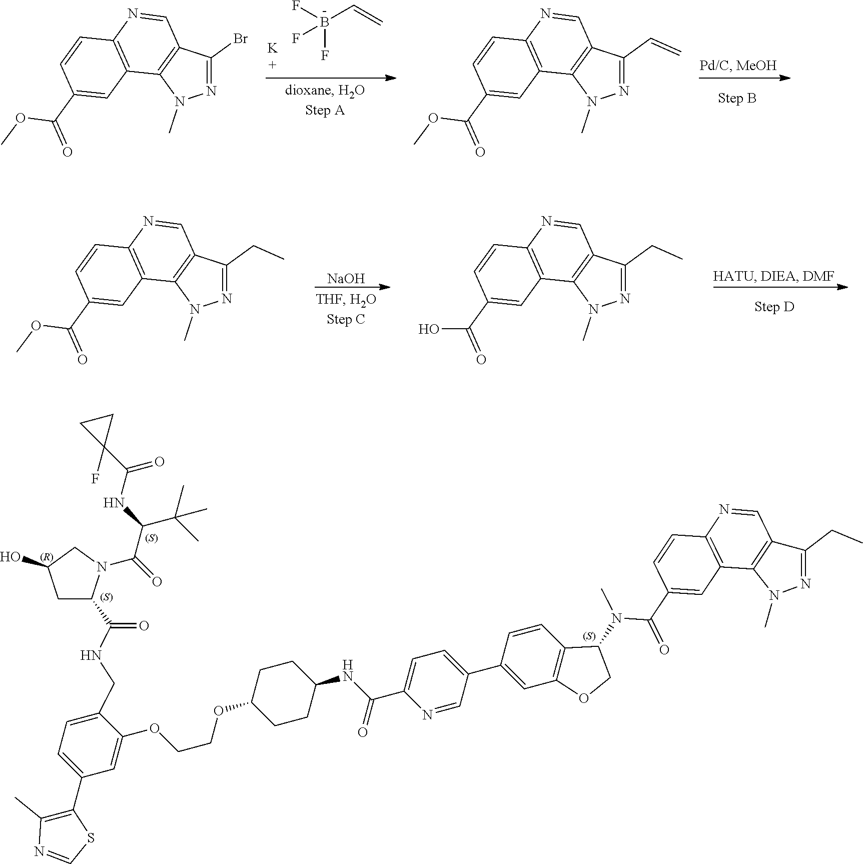

Step A: methyl 1-methyl-3-vinyl-1H-pyrazolo[4,3-c]quinoline-8-carboxylate

To a solution of Potassium trifluoro (vinyl) borate (1-)(84 mg, 0.62 mmol, 2.0 eq.), methyl 3-bromo-1-methyl-1H-pyrazolo[4,3-c]quinoline-8-carboxylate (100 mg, 0.31 mmol, 1.0 eq.), K₂CO₃ (130 mg, 0.94 mmol, 3.0 eq.) in 1,4-Dioxane (5 mL) and H₂O (1 mL) was added PdCl₂ (dppf)(23 mg, 31 μmol, 0.1 eq.) at room temperature. The mixture was stirred at 100° C. for 16 hours under N₂. The reaction was concentrated and purified by silica gel chromatography (100-200 mesh silica gel, 0-30% EtOAc in PE) to give methyl 1-methyl-3-vinyl-1H-pyrazolo[4,3-c]quinoline-8-carboxylate (50 mg, 60% yield) as a yellow solid. LC-MS (ESI): mass calcd. For C₁₅H₁₃N₃O, 267.1; m/z found, 268 [M+H]⁺.

Step B: methyl 3-ethyl-1-methyl-1H-pyrazolo[4,3-c]quinoline-8-carboxylate

To a solution of methyl 1-methyl-3-vinyl-1H-pyrazolo[4,3-c]quinoline-8-carboxylate (50 mg, 187 μmol, 1.0 eq.) and Pd/C (20 mg, 187 μmol, 1.0 eq.) in MeOH (5.0 mL) was stirred at room temperature for 4 hours under H₂. The reaction was filtered and the filtrate was concentrated in vacuo to give the methyl 3-ethyl-1-methyl-1H-pyrazolo[4,3-c]quinoline-8-carboxylate (50 mg, 99% yield) as a yellow solid. LC-MS (ESI): mass calcd. For C₁₅H15N302, 269.1; m/z found, 270

Step C: 3-ethyl-N-(S)-6-(6-(((15,4S)-4-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N, 1-dimethyl-1H-pyrazolo[4,3-c]quinoline-8-car boxamide

Example A330 was synthesized following the same procedures as described in Example A29 (4.3 mg, 22% yield) as a yellow solid. LC-MS (ESI): mass calcd, for C₆₅H₇₂FN₉O₉S, 1173; m/z found, 587.6 [M/2+H]⁺. ¹H NMR (400 MHz, DMSO-d₆) δ 9.70 (s, 1H), 8.99 (s, 1H), 8.71 (s, 1H), 8.50 (t, J=6.1 Hz, 1H), 8.34 (s, 1H), 8.12 (s, 1H), 7.86 (s, 1H), 7.73 (s, 2H), 7.61 (s, 1H), 7.41 (d, J=7.8 Hz, 1H), 7.33 (s, 1H), 7.30-7.20 (m, 2H), 7.06 (s, 1H), 6.98 (d, J=8.9 Hz, 1H), 6.07 (m, 1H), 4.91-4.72 (m, 2H), 4.60-4.52 (m, 5H), 4.47 (s, 2H), 4.35 (s, 1H), 4.31 (d, J=6.2 Hz, 1H), 4.26 (d, J=5.7 Hz, 1H), 4.19 (s, 2H), 4.06-4.02 (m, 1H), 3.84-3.82 (m, 2H), 3.65-3.63 (m, 2H), 3.61 (s, 1H), 3.42 (s, 1H), 3.13 (q, J=7.5 Hz, 2H), 2.79-2.66 (m, 3H), 2.47 (s, 3H), 2.17-2.06 (m, 3H), 1.97-1.90 (m, 1H), 1.84-1.77 (m, 2H), 1.74-1.63 (m, 2H), 1.40 (t, J=7.6 Hz, 3H), 1.37-1.31 (m, 3H), 1.23-1.19 (m, 3H), 0.95 (s, 9H).

### Example A331. 3-cyclopropyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,1-dimethyl-1H-pyrazolo[4,3-c]quinoline-8-carboxamide

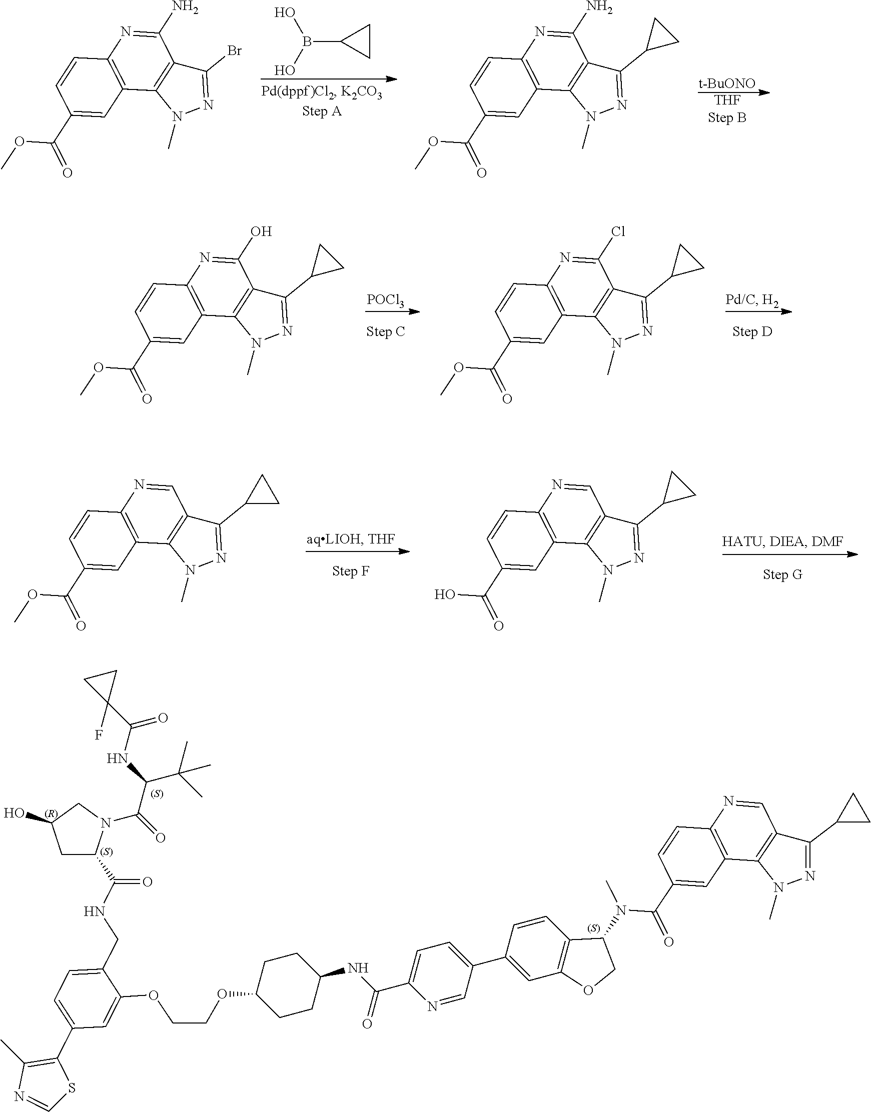

Step A: methyl 4-amino-3-cyclopropyl-1-methyl-1H-pyrazolo[4,3-c]quinoline-8-carboxylate

To a mixture of methyl 4-amino-3-bromo-1-methyl-1H-pyrazolo[4,3-c]quinoline-8-carboxylate (100 mg, 298 μmol, 1.0 eq.), K₂CO₃ (103 mg, 746 μmol, 2.5 eq.) and cyclopropylboronic acid (38 mg, 448 μmol, 1.5 eq.) in 1,4-Dioxane (5 mL) and H₂O (1 mL) was added PdCl₂ (dppf)-CH₂Cl₂ (12 mg, 14.9 μmol, 0.05 eq.) and the mixture was stirred at 100° C. for 3 hours. The mixture was poured into water (50 mL) and extracted with EtOAc (50 mL×3). The combined organic layers were dried over anhydrous Na₂SO₄, filtered and the filtrate was concentrated to dryness. The residue was purified by column chromatography (silica gel, 0% EtOAc in PE~100% EtOAc in PE) to afford methyl 4-amino-3-cyclopropyl-1-methyl-1H-pyrazolo[4,3-c]quinoline-8-carboxylate (40 mg, 45% yield) as a yellow oil. LC-MS (ESI): mass calcd. For C₁₆H₁₆N₄O₂, 296.13; m/z found, 297.0 [M+H]⁺.

Step B: methyl 3-cyclopropyl-4-hydroxy-1-methyl-1H-pyrazolo[4,3-c]quinoline-8-carboxylate

To a solution of methyl 4-amino-3-cyclopropyl-1-methyl-1H-pyrazolo[4,3-c]quinoline-8-carboxylate (130 mg, 439 μmol, 1.0 eq.) and tert-Butyl nitrite (2.6 g, 3 mL, 25.2 mmol, 57.4 eq.) in THF (9 mL) was stirred at 70° C. for 16 hours. The residue was purified by column chromatography (silica gel, 0% EtOAc in PE~30% EtOAc in PE) to give methyl 3-cyclopropyl-4-hydroxy-1-methyl-1H-pyrazolo[4,3-c]quinoline-8-carboxylate (80 mg, 61% yield) as a yellow solid. LC-MS (ESI): mass calcd. For C₁₆H₁₅N₃O₃, 297.11; m/z found, 298.0 [M+H]⁺.

Step C: methyl 4-chloro-3-cyclopropyl-1-methyl-1H-pyrazolo[4,3-c]quinoline-8-carboxylate

To a solution of methyl 3-cyclopropyl-4-hydroxy-1-methyl-1H-pyrazolo[4,3-c]quinoline-8-carboxylate (80 mg, 269 μmol, 1.0 eq.) in POCl₃ (2 mL) was stirred at 100° C. for 1 hour. The mixture was poured into water (30 mL) and extracted with EtOAc (30×3 mL). The combined organic layers were dried over anhydrous Na₂SO₄, filtered and the filtrate was concentrated to dryness. The reaction was purified by column chromatography (silica gel, 0% EtOAc in PE~30% EtOAc in PE) to give methyl 4-chloro-1-ethyl-1H-pyrazolo[4,3-c]quinoline-8-carboxylate (60 mg, 70% yield) as a yellow oil. LC-MS (ESI): mass calcd. For C₁₆H₁₄ClN₃O₂, 315.08; m/z found, 316.1 [M+H]⁺.

Step D: methyl 3-cyclopropyl-1-methyl-1H-pyrazolo[4,3-c]quinoline-8-carboxylate

To the solution of methyl 4-chloro-3-cyclopropyl-1-methyl-1H-pyrazolo[4,3-c]quinoline-8-carboxylate (100 mg, 317 μmol, 1.0 eq.) in MeOH (5 mL) were added palladium (30 mg, 30% Wt) and stirred for 3 hour at 30° C. undr the H2 protection. The residue was filtered and to afford the methyl 3-cyclopropyl-1-methyl-1H-pyrazolo[4,3-c]quinoline-8-carboxylate (60 mg, 67% yield) as a colorless oil. LC-MS (ESI): mass calcd. For C₁₆H₁₅N₃O₂, 281.12; m/z found, 282.1

Steps F-G: 3-cyclopropyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N, 1-dimethyl-1H-pyrazolo[4,3-c]quinoline-8-carboxamide

Example A331 was synthesized following the same procedures as described in Example A29 (2.5 mg, 5% yield over 5 steps) as a white solid. LC-MS (ESI): mass calcd. for C₆₄H₇₁FN₁₀O₉S, 1174.5; m/z found, 1176.3 [M+H]⁺. ¹H NMR (400 MHz, DMSO-d₆) δ 9.62 (s, 1H), 8.99 (s, 1H), 8.92 (s, 1H), 8.66 (s, 1H), 8.50 (t, J=5.9 Hz, 2H), 8.28 (s, 2H), 8.08 (d, J=8.1 Hz, 2H), 7.65 (s, 1H), 7.41 (d, J=7.8 Hz, 2H), 7.34-7.27 (m, 2H), 7.05 (s, 1H), 6.97 (d, J=7.9 Hz, 1H), 4.78 (s, 1H), 4.60 (d, J=9.2 Hz, 1H), 4.49 (s, 3H), 4.36 (s, 2H), 4.30 (d, J=6.0 Hz, 1H), 4.25 (d, J=6.0 Hz, 1H), 4.18 (s, 3H), 3.81 (s, 4H), 3.62 (s, 3H), 2.76 (s, 1H), 2.67 (s, 2H), 2.47 (s, 3H), 2.09-2.01 (m, 4H), 1.96-1.91 (m, 1H), 1.88-1.83 (m, 2H), 1.54-1.47 (m, 2H), 1.37 (s, 1H), 1.35-1.29 (m, 3H), 1.23-1.21 (m, 2H), 1.18-1.14 (m, 2H), 1.10-1.06 (m, 2H), 0.96 (s, 9H).

### Example A334. 3-cyclopropyl-N-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N,1-dimethyl-1H-pyrazolo[4,3-c]quinoline-8-carboxamide

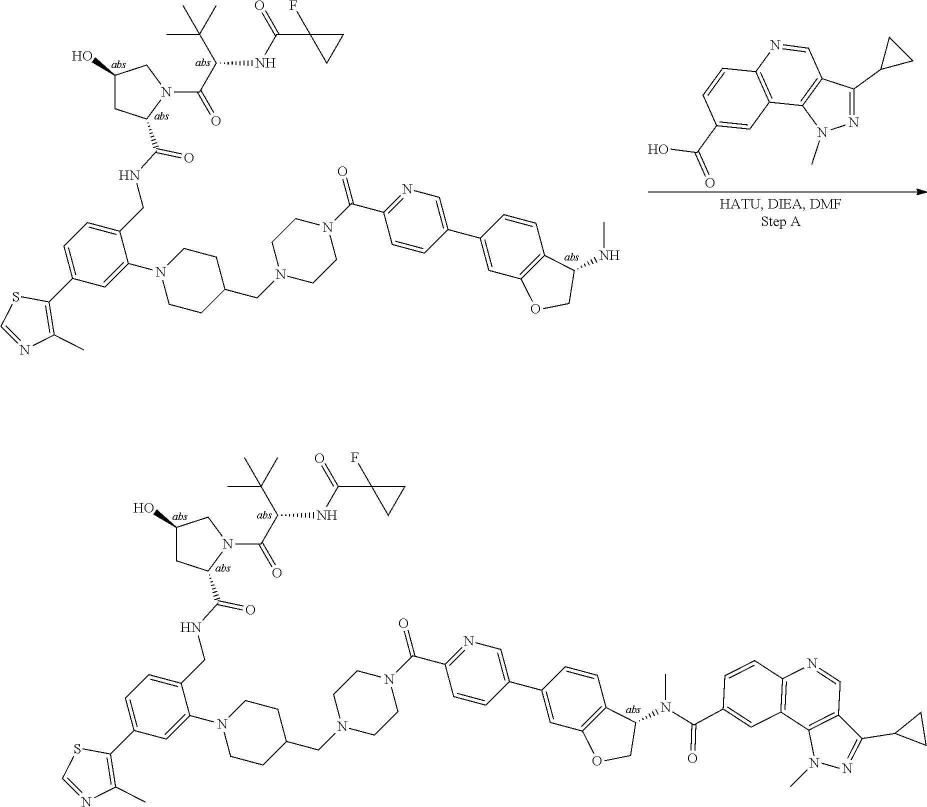 [SMILES: CN[C@@H]1COc2cc(-c3ccc(C(=O)N4CCN(CC5CCN(c6cc(-c7scnc7C)ccc6CNC(=O)[C@@H]6C[C@@H](O)CN6C(=O)[C@@H](NC(=O)C6(F)CC6)C(C)(C)C)CC5)CC4)nc3)ccc21 ; Cn1nc(C2CC2)c2cnc3ccc(C(=O)O)cc3c21 ; Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)c5ccc6ncc7c(C8CC8)nn(C)c7c6c5)cn4)CC3)CC2)c1]

Example A334 was synthesized following the same procedures as described in Example A114 (2.4 mg, 12% yield) as a white solid. LC-MS (ESI): mass calcd, for C₆₆H₇₅FN₁₂O₇S, 1198.56; m/z found, 1200.3 [M+H]⁺. ¹H NMR (400 MHz, DMSO-d₆) δ 9.72 (s, 1H), 8.99 (s, 1H), 8.93 (s, 1H), 8.69 (s, 1H), 8.52 (t, J=5.6 Hz, 1H), 8.36-8.26 (m, 2H), 8.10 (s, 1H), 7.78 (d, J=8.1 Hz, 1H), 7.69-7.60 (m, 1H), 7.46-7.34 (m, 3H), 7.30-7.25 (m, 1H), 7.16-7.12 (m, 2H), 4.79 (s, 1H), 4.60 (d, J=9.4 Hz, 2H), 4.49 (dd, J=11.0, 4.7 Hz, 5H), 4.37 (s, 3H), 3.20-3.09 (m, 8H), 2.73 (dd, J=23.8, 13.4 Hz, 6H), 2.63-2.57 (m, 2H), 2.46 (s, 3H), 2.33 (s, 1H), 2.14-2.03 (m, 1H), 2.04-1.97 (m, 1H), 1.95-1.86 (m, 4H), 1.49-1.42 (m, 2H), 1.41-1.37 (m, 1H), 1.36-1.33 (m, 1H), 1.24-1.22 (m, 1H), 1.22-1.19 (m, 2H), 1.19-1.16 (m, 1H), 1.11-1.08 (m, 2H), 0.98 (s, 9H).

### Example A335. 2-amino-3-ethynyl-N-((S)-6-(6-(((1S,3R,4S)-3-fluoro-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-N-methylquinoline-6-carboxamide

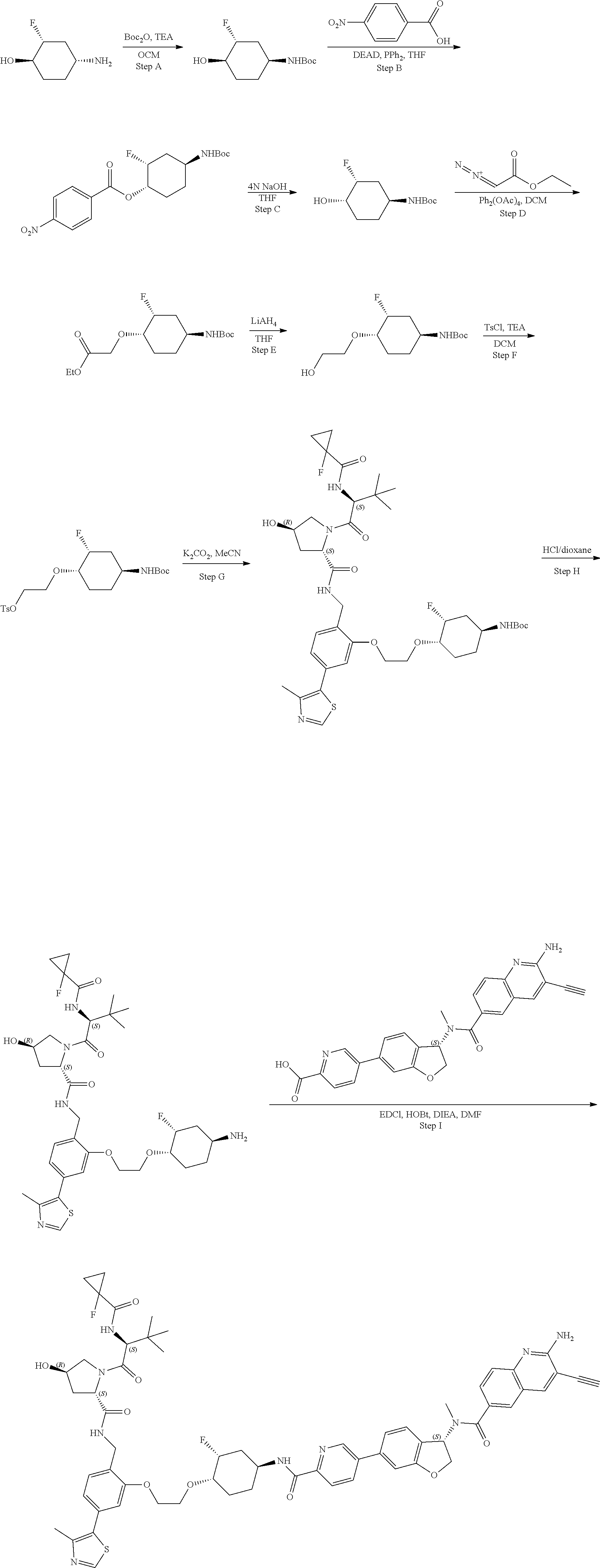

Step A: tert-butyl ((1S,3R, 4R)-3-fluoro-4-hydroxycyclohexyl)carbamate

A mixture of (1R,2R,4S)-4-amino-2-fluorocyclohexan-1-ol (1.7 g, 12.8 mmol, 1.0 eq.) (WO2021186324), Boc₂O (5.6 g, 25.5 mmol, 2.0 eq.) and TEA (5.1 mL, 38.3 mmol, 3.0 eq.) in DCM (30 mL) was stirred at room temperature for 2 hours under nitrogen. The resulting solution was diluted with 50 mL of water and extracted with EtOAc (50 mL×3). The combined organic layers were washed with brine, dried over Na₂SO₄ and concentrated under reduced pressure. The crude product was purified by flash column chromatography on silica gel (100-200 mesh silica gel, 0~20% EtOAc in PE) to afford tert-butyl ((1S,3R,4R)-3-fluoro-4-hydroxycyclohexyl)carbamate (1.70 g, 57% yield) as a yellow solid. LC-MS (ESI): mass calcd. For C₁₁H₂₀FNO₃, 233.14; m/z found, 234 [M+H]⁺.

Step B: (1S,2R,4S)-4-((tert-butoxycarbonyl)amino)-2-fluorocyclohexyl 4-nitrobenzoate

A mixture of tert-butyl ((1S,3R,4R)-3-fluoro-4-hydroxycyclohexyl)carbamate (300 mg, 1.29 mmol, 1.0 eq.), 4-nitrobenzoic acid (322 mg, 1.93 mmol, 1.5 eq.), PPh₃ (842 mg, 3.22 mmol, 2.5 eq.) and DIAD (649 mg, 3.22 mmol, 2.5 eq.) in THF (5 mL) was stirred at room temperature for 16 hours under nitrogen. The resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~20% EtOAc in PE) to afford methyl 2-(bis(tert-butoxycarbonyl)amino)-3-formylquinoline-6-carboxylate (420 mg, 85% yield) as a white solid. LC-MS (ESI): mass calcd. For C₁₈H23FN206, 382.15; m/z found, 383 [M+H]⁺.

Step C: tert-butyl ((1S,3R,4S)-3-fluoro-4-hydroxycyclohexyl)carbamate

A mixture of (1S,2R,4S)-4-((tert-butoxycarbonyl)amino)-2-fluorocyclohexyl 4-nitrobenzoate (420 mg, 1.1 mmol, 1.0 eq.) and 4N NaOH (1.5 mL) in THF (5 mL) was stirred at room temperature for 1 hour under nitrogen. After cooling to room temperature, the resulting solution was diluted with 50 mL of water and extracted with EtOAc (50 mL×3). The combined organic layers were washed with brine (50 mL), dried over Na₂SO₄ and concentrated under reduced pressure. The crude product was purified by flash column chromatography on silica gel (100-200 mesh silica gel, 0~50% EtOAc in PE) to afford tert-butyl (15,3R,4S)-3-fluoro-4-hydroxycyclohexyl)carbamate (150 mg, 58% yield) as a yellow solid. LC-MS (ESI): mass calcd. For C₁₁H₂₀FNO₃, 233.14; m/z found, 234 [M+H]⁺

Steps D-1:2-amino-3-ethynyl-N-((S)-6-(6-(((1S,3R,4S)-3-fluoro-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

Example A335 was synthesized following the same procedures as described in Example A220 (9.7 mg, 2% yield over 6 steps) as a white solid. LC-MS (ESI): mass calcd. for C₆₁H₆₅F₂NO₉S, 1137.46; m/z found, 1139 [M+H]⁺. ¹H NMR (400 MHz, DMSO-d₆) δ 9.00 (s, 1H), 8.93 (s, 2H), 8.72 (s, 1H), 8.64 (d, J=8.7 Hz, 1H), 8.51 (t, J=5.9 Hz, 1H), 8.28 (d, J=8.2 Hz, 1H), 8.13-8.01 (m, 2H), 7.93 (d, J=8.6 Hz, 1H), 7.77 (s, 1H), 7.57 (d, J=7.7 Hz, 1H), 7.47-7.25 (m, 4H), 7.06 (s, 1H), 6.98 (d, J=7.9 Hz, 1H), 5.14-4.99 (m, 2H), 4.85-4.78 (m, 1H), 4.71-4.69 (m, 1H), 4.61-4.59 (m, 1H), 4.55-4.53 (m, 1H), 4.36 (s, 1H), 4.31-4.27 (m, 2H), 4.20 (d, J=4.3 Hz, 2H), 4.15-4.13 (m, 1H), 3.91-3.82 (m, 3H), 3.68-3.60 (m, 2H), 3.55-3.49 (m, 1H), 2.67 (s, 3H), 2.48 (s, 3H), 2.17-2.08 (m, 2H), 1.97-1.81 (m, 4H), 1.70-1.56 (m, 2H), 1.41-1.29 (m, 2H), 1.24-1.17 (m, 2H), 0.97 (s, 9H).

### Example A336. 2-amino-3-ethynyl-N-((3S)-6-(6-(3-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)cyclobutyl)-3,8-diazabicyclo[3.2.1]octane-8-carbonyl)pyridin-3-yl)-2,3-dihydrobenzofuran-3-yl)-N-methylquinoline-6-carboxamide

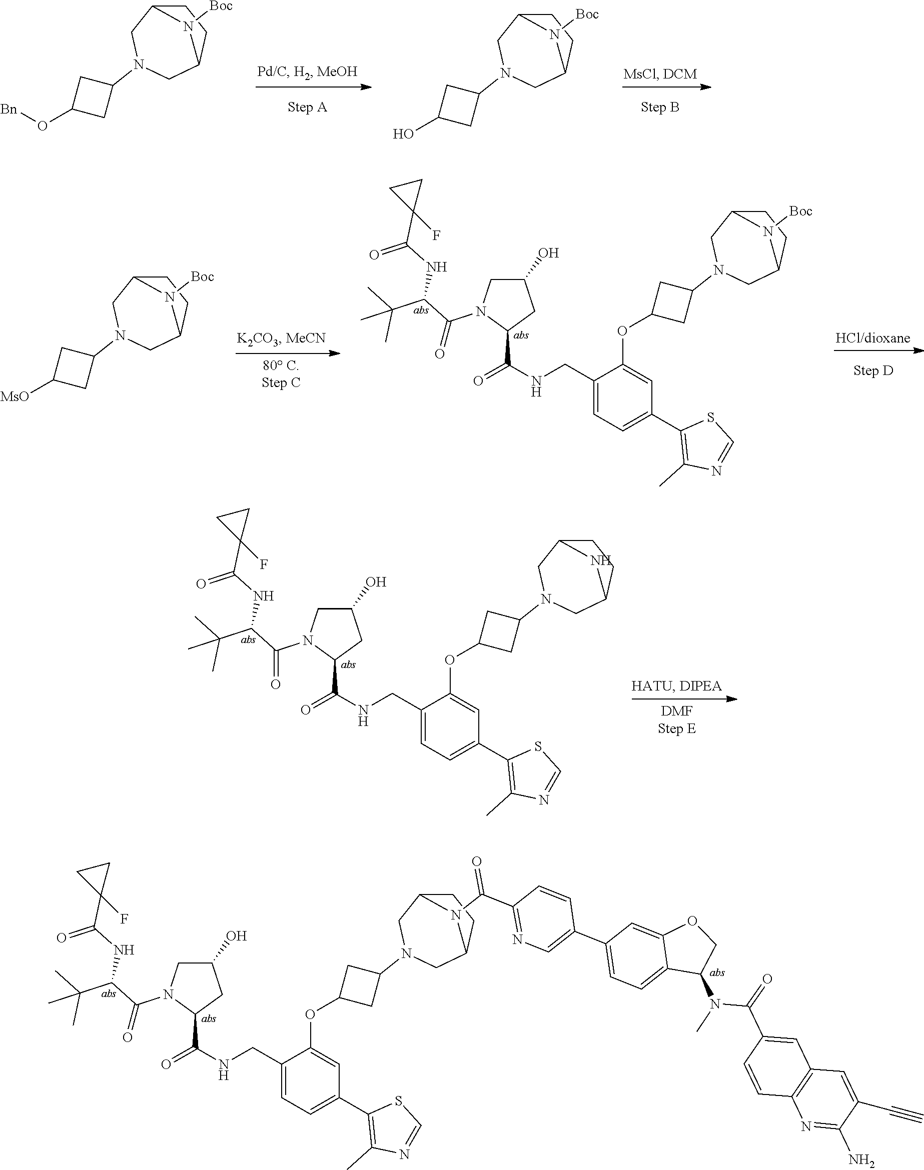

Step A: tert-butyl 3-(3-hydroxycyclobutyl)-3,8-diazabicyclo[3.2.1]octane-8-carboxylate

To a stirred mixture of tert-butyl 3-(3-(benzyloxy)cyclobutyl)-3,8-diazabicyclo[3.2.1]octane-8-carboxylate (500 mg, 1.34 mmol, 1.0 eq.) in MeOH (5 mL) was added Pd/C (143 mg, 1.34 mmol, 1.0 eq.). The resulting mixture was stirred at room temperature for 15 hours under H₂. The reaction mixture was evaporated and purified by column chromatography (silica gel, 0% EtOAc in PE~20% EtOAc in PE) to afford tert-butyl 3-(3-hydroxycyclobutyl)-3,8-diazabicyclo[3.2.1]octane-8-carboxylate (300 mg, 79% yield) as a yellow solid. LC-MS (ESI): mass calcd. For C₁₅H₂₆N₂O₃, 282.19; m/z found, 283.3 [M+H]⁺.

Step B: tert-butyl 3-(3-(methylsulfonyl)oxy)cyclobutyl)-3,8-diazabicyclo[3.2.1]octane-8-carboxylate

To a stirred mixture of tert-butyl 3-(3-hydroxycyclobutyl)-3,8-diazabicyclo[3.2.1]octane-8-carboxylate (200 mg, 708 μmol, 1.0 eq.) and TEA (215 mg, 296 μL, 2.12 mmol, 3.0 eq.) in DCM (4 mL) were added Methanesulfonyl chloride (162 mg, 1.42 mmol, 2.0 eq.) at 0° C. The resulting mixture was stirred at room temperature for 4 hours. The reaction mixture was poured into water (20 mL), and then extracted with EtOAc (20 mL×2). The combined organic layers were washed with brine, dried over anhydrous Na₂SO₄ and concentrated. The residue was purified by column chromatography (silica gel, 0% EtOAc in PE~30% EtOAc in PE) to afford tert-butyl 3-(3-((methylsulfonyl)oxy)cyclobutyl)-3,8-diazabicyclo[3.2.1]octane-8-carboxylate (200 mg, 78% yield) as a yellow solid. LC-MS (ESI): mass calcd. For C₁₆H28N205S, 360.17; m/z found, 361.4 [M+H]⁺.

Step C-E: 2-amino-3-ethynyl-N-(3S)-6-(6-(3-(3-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)cyclobutyl)-3,8-diazabicyclo[3.2.1]octane-8-carbonyl)pyridin-3-yl)-2,3-dihydrobenzofuran-3-yl)-N-methylquinoline-6-carboxamide

Example A336 was synthesized following the same procedures as described in Example A29 (5.4 mg, 2% yield over 3 steps) as a white solid. LC-MS (ESI): mass calcd. for C₆₃H₆₇FN100&S, 1142.48; m/z found, 1143.5 [M+H]⁺. ¹H NMR (400 MHz, DMSO-d₆) δ 10.00 (s, 1H), 9.00 (s, 1H), 8.94 (s, 1H), 8.64 (s, 1H), 8.51 (t, J=6.0 Hz, 1H), 8.31-8.27 (m, 1H), 8.05 (s, 1H), 7.94 (d, J=8.3 Hz, 1H), 7.88 (d,)=10.1 Hz, 1H), 7.77-7.68 (m, 1H), 7.56 (d, J=7.8 Hz, 1H), 7.45-7.38 (m, 2H), 7.35 (s, 1H), 7.29-7.23 (m, 1H), 6.99 (d, J=8.9 Hz, 1H), 6.77 (s, 1H), 6.38 (s, 1H), 5.64 (s, 1H), 5.17 (s, 1H), 5.02-4.96 (m, 1H), 4.94-4.88 (m, 2H), 4.86-4.72 (m, 1H), 4.71-4.67 (m, 1H), 4.61-4.56 (m, 1H), 4.52 (t, J=8.1 Hz, 1H), 4.38-4.21 (m, 4H), 3.60-3.59 (m, 2H), 3.47-3.46 (m, 1H), 3.25-3.09 (m, 3H), 2.93-2.84 (m, 2H), 2.67 (s, 3H), 2.46-2.37 (m, 6H), 2.18-1.97 (m, 6H), 1.94-1.88 (m, 1H), 1.41-1.31 (m, 2H), 1.23-1.18 (m, 2H), 0.94 (s, 9H).

### Example A337. 2-amino-3-ethynyl-N-((3S)-6-(6-(3-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)phenyl)-3,8-diazabicyclo[3.2.1]octane-8-carbonyl)pyridin-3-yl)-2,3-dihydrobenzofuran-3-yl)-N-methylquinoline-6-carboxamide

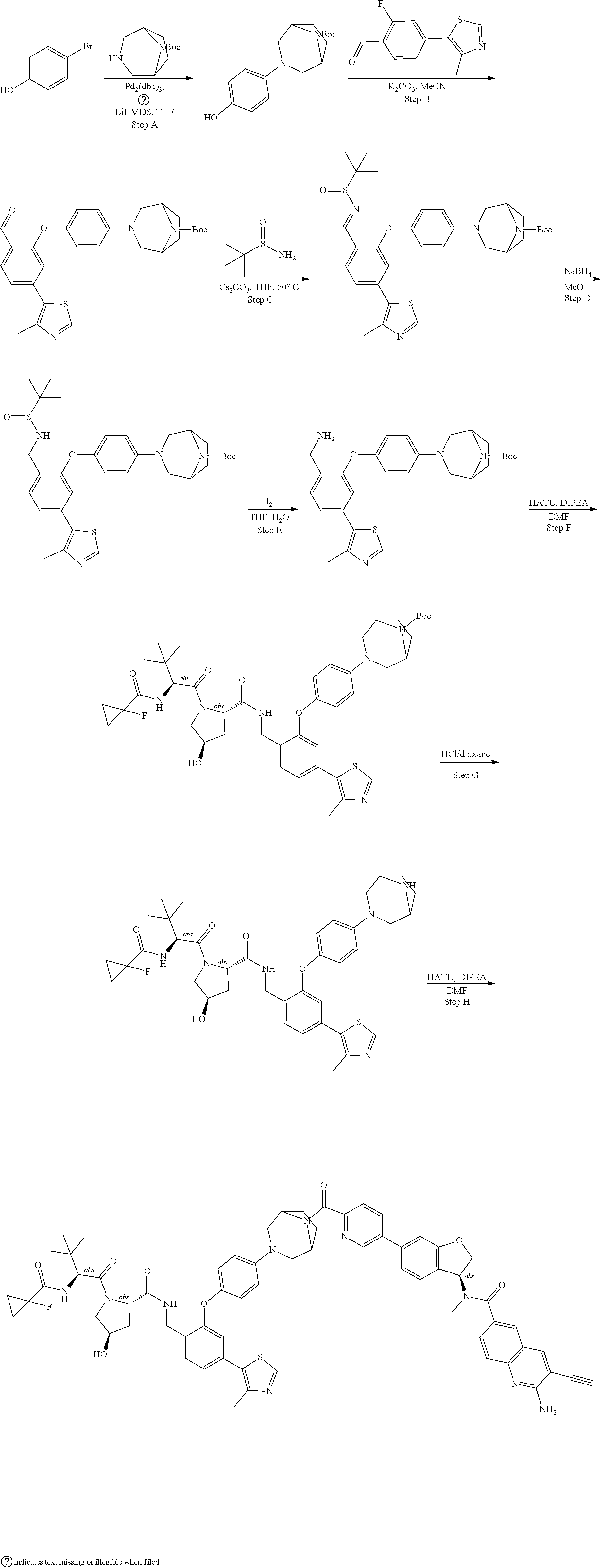

Example A337 was synthesized following the same procedures as described in Example A126 (4.0 mg, 7% yield over 8 steps) as a white solid. LC-MS (ESI): mass calcd. for C₆₅H₆₅FN100&S, 1164.47; m/z found, 1165.5 [M+H]⁺. ¹H NMR (400 MHz, DMSO-d₆) δ 8.94 (s, 1H), 8.92 (d, J=1.6 Hz, 1H), 8.67-8.58 (m, 2H), 8.34-8.15 (m, 3H), 8.04 (s, 1H), 7.87 (d, J=8.3 Hz, 1H), 7.73 (s, 1H), 7.65 (d, J=8.2 Hz, 1H), 7.54 (dd, J=7.8, 3.3 Hz, 2H), 7.39 (d, J=7.7 Hz, 1H), 7.34 (s, 1H), 7.29 (dd, J=9.1, 2.5 Hz, 1H), 7.10 (dd, J=7.9, 1.6 Hz, 1H), 7.02-6.95 (m, 4H), 6.73 (d, J=1.6 Hz, 1H), 6.38 (s, 1H), 4.91 (s, 1H), 4.80 (s, 1H), 4.72-4.65 (m, 2H), 4.60 (d, J=9.4 Hz, 1H), 4.51 (t, J=8.3 Hz, 1H), 4.44-4.38 (m, 1H), 4.37-4.33 (m, 1H), 4.24-4.15 (m, 2H), 3.64-3.62 (m, 2H), 3.46 (s, 2H), 3.18-3.13 (m, 3H), 2.67 (s, 3H), 2.34 (s, 3H), 2.15-2.05 (m, 1H), 2.03-1.97 (m, 2H), 1.93-1.89 (m, 2H), 1.84-1.76 (m, 1H), 1.42-1.32 (m, 2H), 1.25-1.19 (m, 2H), 0.97 (s, 9H).

### Example A338. 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-3-(4-hydroxybutyl)-N-methylquinoline-6-carboxamide

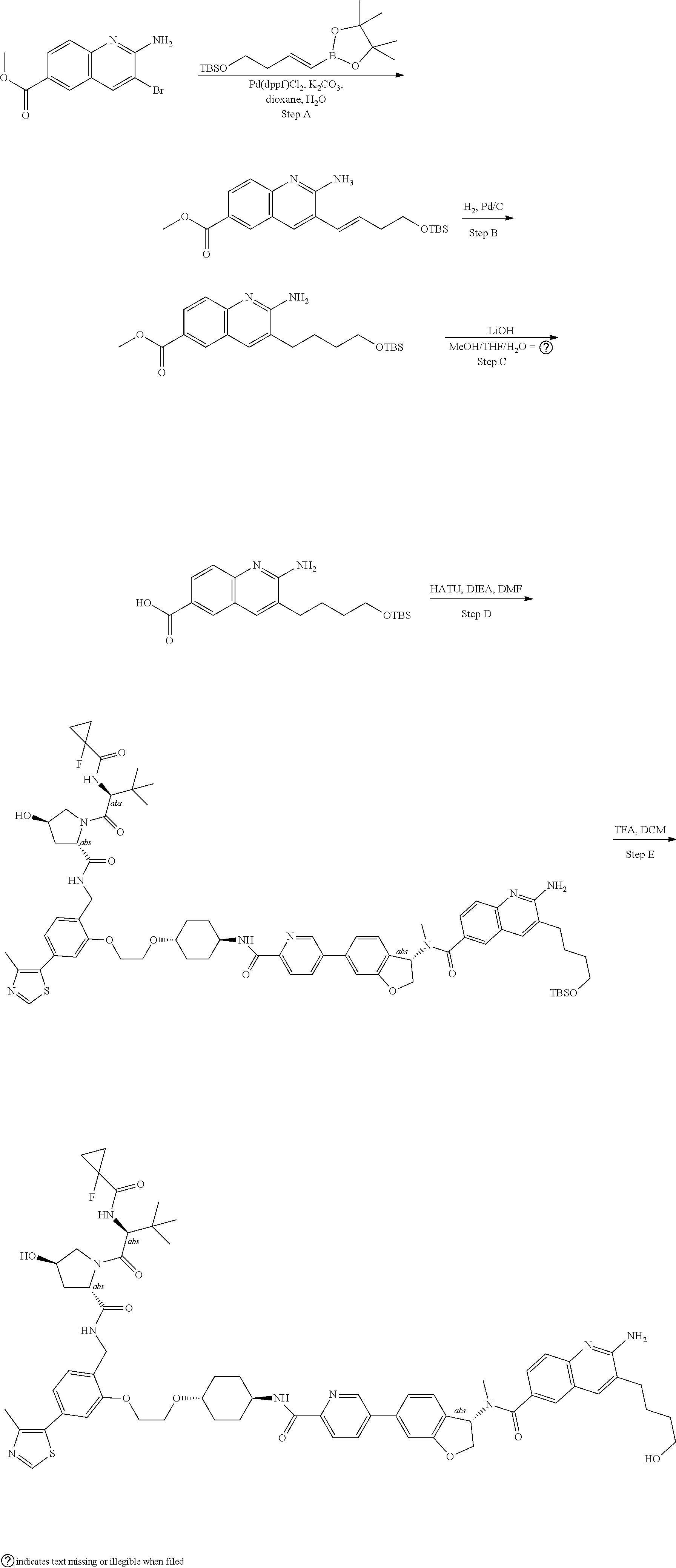

Step A: methyl (E)-2-amino-3-(4-((tert-butyldimethylsilyl)oxy)but-1-en-1-yl)quinoline-6-carboxylate

To the solution of methyl 2-amino-3-bromoquinoline-6-carboxylate (300 mg, 1.07 mmol, 1 eq) in 1,4-Dioxane (10 mL) and water (1 mL) were added PdCl₂ (dppf)(78.1 mg, 107 μmol, 0.1 eq.), (E)-tert-butyldimethyl ((4-(4,4,5,5-tetramethyl-1,3,2-dioxaborolan-2-yl)but-3-en-1-yl)oxy) silane (400 mg, 1.28 mmol, 1.2 eq.) and K₂CO₃ (442 mg, 3.20 mmol, 3.0 eq.). The resulting mixture was stirred at 120° C. for 15 hours under N₂ protection. The residue was filtered and purified by column chromatography (silica gel, 0~30% EtOAc in PE) to afford the methyl (E)-2-amino-3-(4-((tert-butyldimethylsilyl)oxy)but-1-en-1-yl)quinoline-6-carboxylate (410 mg, 99% yield) as a yellow oil. LC-MS (ESI): mass calcd. For C₂₁H₃₀N₂O₃Si, 386.20; m/z found, 387.20 [M+H]⁺

Step B: methyl 2-amino-3-(4-((tert-butyldimethylsilyl)oxy)butyl)quinoline-6-carboxylate

To the solution of methyl (E)-2-amino-3-(4-((tert-butyldimethylsilyl)oxy)but-1-en-1-yl)quinoline-6-carboxylate (410 mg, 1.06 mmol, 1.0 eq.) in MeOH (10 mL) was added Pd/C (226 mg, 2.12 mmol, 2.0 eq.). The resulting mixture was stirred at room temperature for 1 hours under the H2 protection. LCMS showed this reaction was completed. The residue was filtered and concentrated under reduced pressure to afford the methyl 2-amino-3-(4-((tert-butyldimethylsilyl)oxy)butyl)quinoline-6-carboxylate (410 mg, 99% yield) as a colorless oil. LC-MS (ESI): mass calcd. For C₂₁H32N₂O₃Si, 388.22; m/z found, 389.22 [M+H]⁺.

Step C: 2-amino-3-(4-((tert-butyldimethylsilyl)oxy)butyl)quinoline-6-carboxylic acid

To the solution of methyl 2-amino-3-(4-((tert-butyldimethylsilyl)oxy)butyl)quinoline-6-carboxylate (410 mg, 1.06 mmol, 1.0 eq.) in MeOH (4 mL) and THF (4 mL) were added lithium hydroxide (50.5 mg, 2.11 mmol, 2.0 eq.) in Water (1 mL) at 0° C. The resulting mixture was stirred at room temperature for 1 hours. LCMS showed this reaction was completed. The residue was concentrated under reduced pressure to afford the 2-amino-3-(4-((tert-butyldimethylsilyl)oxy)butyl)quinoline-6-carboxylic acid (390 mg, 98% yield) as a yellow solid, which was used for next step without any further purification. LC-MS (ESI): mass calcd. For C20H₃₀N₂O₃Si, 374.20; m/z found, 375.20 [M+H]⁺.

Step D: 2-amino-3-(4-((tert-butyldimethylsilyl) oxy) butyl)-N-(S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

To the solution of 2-amino-3-(4-((tert-butyldimethylsilyl)oxy)butyl)quinoline-6-carboxylic acid (12.0 mg, 1 eq, 32.0 μmol) in DMF (2.00 mL) were added HATU (24.4 mg, 2 eq, 64.1 μmol), N-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy)ethoxy)cyclohexyl)-5-((S)-3-(methylamino)-2,3-dihydrobenzofuran-6-yl)picolinamide (29.7 mg, 1 eq, 32.0 μmol) and DIEA (12.4 mg, 16.7 μL, 3 eq, 96.1 μmol) at 0° C. and stirred for 30 min at 25° C. The residue was extracted with EA and purified by silica gel cholumn chromatography (MeOH/DCM=0~10%) to afford the 2-amino-3-(4-((tert-butyldimethylsilyl)oxy)butyl)-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide (35.0 mg, 27.3 μmol, 85.2%) as a yellow oil.

LCMS: 1282 [M+1]; retention time: 2.287 min. Step E: 2-amino-N-(S)-6-16-(((1S, 4S)-4-(2-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-3-(4-hydroxybutyl)-N-methylquinoline-6-carboxamide

To the solution of 2-amino-3-(4-((tert-butyldimethylsilyl)oxy)butyl)-N-((S)-6-(6-(((1S,4S)-4-(2-(2-(((2S,4R)-1-((S)-2-(1-fluorocyclopropane-1-carboxamido)-3,3-dimethyl butanoyl)-4-hydroxypyrrolidine-2-carboxamido)methyl)-5-(4-methylthiazol-5-yl)phenoxy) ethoxy)cyclohexyl)carbamoyl)pyridin-3-yl)-2,3-dihydrobenzofuran-3-yl)-N-methylquinoline-6-carboxamide (50.0 mg, 1 eq, 39.0 μmol) in DCM (2.00 mL) were added 4 N HCl in dioxane (2.00 mL) at 0° C. and stirred for 30 min at 25° C. The residue was purified by prep-HPLC (MeCN/0.05% TFA=5~95%) to afford the 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-3-(4-hydroxybutyl)-N-methylquinoline-6-carboxamide (30.3 mg, 25 μmol, 63% yield, 95% purity) as a colorless oil. LC-MS (ESI): mass calcd. For C₆₃H₇₄FN₉O₁₀S, 1167.53; m/z found, 1168.53 [M+H]⁺. ¹H NMR (400 MHz, DMSO-d₆) δ 8.99 (s, 1H), 8.93 (d, J=1.8 Hz, 1H), 8.53-8.44 (m, 2H), 8.27 (dd, J=8.2, 2.2 Hz, 1H), 8.08 (d, J=8.2 Hz, 1H), 7.81 (d, J=8.1 Hz, 2H), 7.56 (dd, J=8.5, 1.9 Hz, 2H), 7.49 (d, J=8.5 Hz, 1H), 7.40 (t, J=9.1 Hz, 2H), 7.34 (s, 1H), 7.29 (dd, J=9.3, 2.6 Hz, 1H), 7.05 (d, J=1.4 Hz, 1H), 6.97 (dd, J=7.8, 1.4 Hz, 1H), 6.51 (s, 2H), 5.18 (s, 1H), 4.76 (s, 1H), 4.68 (dd, J=10.2, 4.1 Hz, 1H), 4.60 (d, J=9.2 Hz, 1H), 4.52 (t, J=8.2 Hz, 1H), 4.44 (d, J=20.2 Hz, 1H), 4.32 (dd, J=17.0, 6.6 Hz, 2H), 4.26-4.13 (m, 3H), 3.86-3.76 (m, 3H), 3.64 (dt, J=22.1, 7.3 Hz, 2H), 3.46 (t, J=6.4 Hz, 2H), 3.20-3.10 (m, 1H), 2.68 (s, 3H), 2.58 (t, J=7.5 Hz, 2H), 2.47 (s, 3H), 2.12-2.00 (m, 3H), 1.94 (ddd, J=13.1, 8.9, 4.5 Hz, 1H), 1.86 (d, J=10.5 Hz, 2H), 1.65 (dd, J=15.0, 8.0 Hz, 2H), 1.53 (dd, J=14.4, 7.1 Hz, 4H), 1.38 (d, J=9.3 Hz, 1H), 1.35-1.29 (m, 3H), 1.22 (dd, J=8.7, 3.2 Hz, 2H), 0.96 (s, 9H).

### Example A339. 2-amino-3-ethynyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxo-1,2,3,4-tetrahydroisoquinolin-6-yl)-2,3-dihydrobenzofuran-3-yl)-N-methylquinoline-6-carboxamide

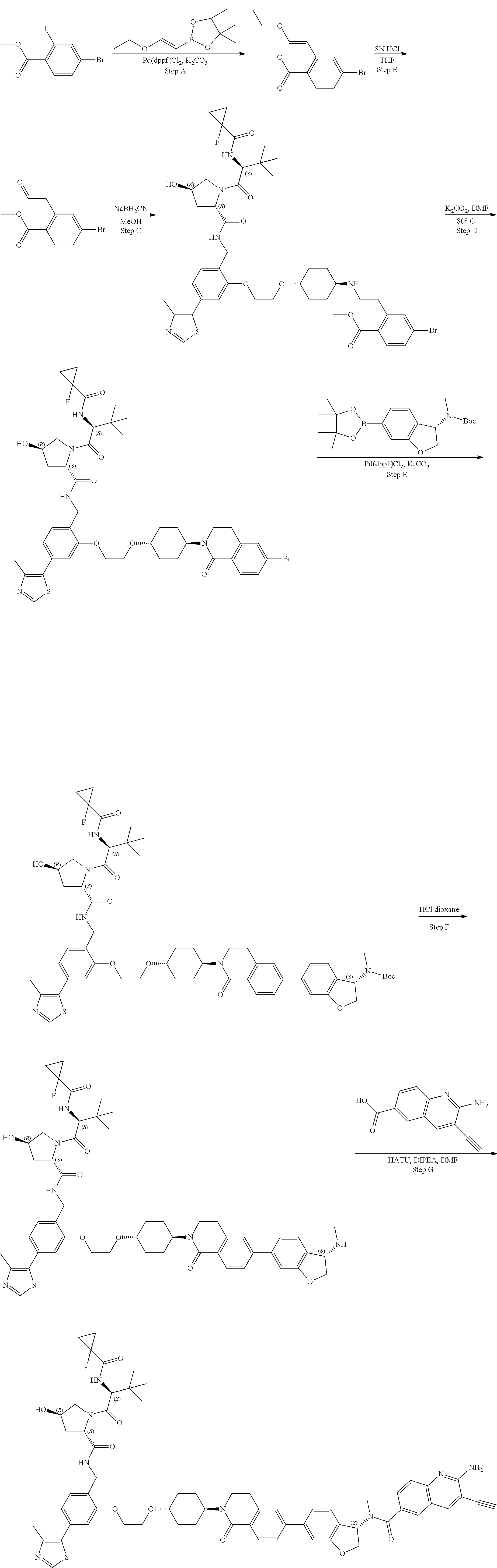

Step A: methyl (E)-4-bromo-2-(2-ethoxyvinyl)benzoate

A mixture of methyl 4-bromo-2-iodobenzoate (3.0 g, 8.8 mmol, 1.0 eq.), (E)-2-(2-ethoxyvinyl)-4,4,5,5-tetramethyl-1,3,2-dioxaborolane (2.3 g, 11.4 mmol, 1.3 eq.), Pd (dppf) C12 (643 mg, 880 μmol, 0.1 eq.) and K₂CO₃ (3.6 g, 26.4 mmol, 3.0 eq.) in 1,4-Dioxane (30 mL) and H₂O (3 mL) was stirred at 80° C. for 6 hours under nitrogen. After cooling to room temperature, the resulting solution was diluted with 50 mL of water and extracted with EtOAc (30 mL×3). The combined organic layers were washed with brine (50 mL), dried over Na₂SO₄ and concentrated under reduced pressure. The crude product was purified by flash column chromatography on silica gel (100-200 mesh silica gel, 0~10% EtOAc in PE) to afford methyl (E)-4-bromo-2-(2-ethoxyvinyl)benzoate (1.0 g, 40% yield) as a colorless oil. LC-MS (ESI): mass calcd. For C₁₂H₁₃BrO₃, 284.00; m/z found, 285 [M+H]⁺.

Step B: methyl 4-bromo-2-(2-oxoethyl)benzoate

A mixture of methyl (E)-4-bromo-2-(2-ethoxyvinyl)benzoate (600 mg, 2.1 mmol, 1.0 eq.) and aq·HCl (6N, 1 mL) in THF (1 mL) was stirred at room temperature for 2 hours under nitrogen. The resulting solution was diluted with 20 mL of aq. NaHCO₃ and extracted with EtOAc (30 mL×3). The combined organic layers were washed with brine (50 mL), dried over Na₂SO₄ and concentrated under reduced pressure. The crude product was purified by flash column chromatography on silica gel (100-200 mesh silica gel, 0~10% EtOAc in PE) to afford methyl 4-bromo-2-(2-oxoethyl)benzoate (200 mg, 37% yield) as a yellow Colorless. LC-MS (ESI): mass calcd. For C₁₀H₁₀BrO₃, 255.97; m/z found, 257 [M+H]⁺.

Step C: methyl 4-bromo-2-(2-(((1S,4r)-4-(2-(2-(((25, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)amino)ethyl)benzoate

A mixture of (2S,4R)-N-(2-(2-(((1r,4S)-4-aminocyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (150 mg, 223 μmol, 1.0 eq.), methyl 4-bromo-2-(2-oxoethyl)benzoate (111 mg, 446 μmol, 2.0 eq.) and HOAc (13 mg, 223 μmol, 1.0 eq.) in MeOH (5 mL) was stirred at room temperature for 30 min. Then NaBH₃CN (114 mg, 445 μmol, 2.0 eq.) was added. The mixture was stirred at room temperature for 16 hrs under nitrogen. The resulting solution was removed under reduced pressure to afford methyl 4-bromo-2-(2-(((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)amino)ethyl)benzoate (150 mg, 74% yield) as a yellow solid. LC-MS (ESI): mass calcd. For C₄₄H₅₇BrFN₅O₈S, 913.31; m/z found, 914 [M+H]⁺.

Step D: (2S,4R)-N-(2-(2-(((1r,4S)-4-(6-bromo-1-oxo-3,4-dihydroisoquinolin-2 (1H)-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

A mixture of methyl 4-bromo-2-(2-(((1S,4r)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)amino)ethyl)benzoate (150 mg, 164 μmol, 1.0 eq.) and K₂CO₃ (68 mg, 492 μmol, 3.0 eq.) in DMF (5 mL) was stirred at 80° C. for 16 hours under nitrogen. The resulting solution was diluted with 20 mL of water and extracted with EtOAc (50 mL×2). The combined organic layers were washed with brine (30 mL), dried over Na₂SO₄ and concentrated under reduced pressure. The crude product was purified by flash column chromatography on silica gel (100-200 mesh silica gel, 0~10% MeOH in DCM) to afford (2S,4R)-N-(2-(2-(((1r,4S)-4-(6-bromo-1-oxo-3,4-dihydroisoquinolin-2 (1H)-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (80.0 mg, 55% yield) as a yellow solid. LC-MS (ESI): mass calcd. For C₄₃H₅₃BrFN₅O₇S, 881.28; m/z found, 882 [M+H]⁺.

Step E: tert-butyl ((S)-6-(2-((1S,4S)-4-(2-(2-(((2S, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxo-1,2,3,4-tetrahydroisoquinolin-6-yl)-2,3-dihydrobenzofuran-3-yl)(methyl)carbamate

A mixture of (2S,4R)-N-(2-(2-(((1r,4S)-4-(6-bromo-1-oxo-3,4-dihydroisoquinolin-2 (1H)-yl)cyclohexyl)oxy)ethoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (80 mg, 90.6 μmol, 1.0 eq.), tert-butyl(S)-methyl (6-(4,4,5,5-tetramethyl-1,3,2-dioxaborolan-2-yl)-2,3-dihydrobenzofuran-3-yl)carbamate (51 mg, 136 μmol, 1.5 eq.), Pd(dppf)Cl₂ (7 mg, 9.06 μmol, 0.1 eq.) and K₂CO₃ (37.5 mg, 272 μmol, 3.0 eq.) in 1,4-Dioxane (1 mL) and H₂O (0.1 mL) was stirred at 100° C. for 3 hour under nitrogen. The resulting solution was diluted with 20 mL of water and extracted with EtOAc (50 mL×2). The combined organic layers were washed with brine (20 mL), dried over Na₂SO₄ and concentrated under reduced pressure. The crude product was purified by flash column chromatography on silica gel (100-200 mesh silica gel, 0~10% MeOH in DCM) to afford tert-butyl ((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxo-1,2,3,4-tetrahydroisoquinolin-6-yl)-2,3-dihydrobenzofuran-3-yl)(methyl)carbamate (40 mg, 42% yield) as a yellow solid. LC-MS (ESI): mass calcd. For C₅₇H₇₁FN6010S, 1050.49; m/z found, 1051 [M+H]⁺.

Steps G-H: 2-amino-3-ethynyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxo-1,2,3,4-tetrahydroisoquinolin-6-yl)-2,3-dihydrobenzofuran-3-yl)-N-methylquinoline-6-carboxamide

Example A339 was synthesized following the same procedures as described in Example A29 (4 mg, 6% yield over 2 steps) as a white solid. LC-MS (ESI): mass calcd, for C₆₄H₆₉FN₈O₉S, 1144.49; m/z found, 1145 [M+H]⁺. ¹H NMR (400 MHz, DMSO-d₆) δ 8.99 (s, 1H), 8.68 (s, 2H), 8.50 (t, J=6.0 Hz, 1H), 8.07 (s, 1H), 7.96-7.88 (m, 2H), 7.75 (s, 1H), 7.66-7.57 (m, 2H), 7.50 (d, J=7.9 Hz, 1H), 7.41 (d, J=7.8 Hz, 1H), 7.36-7.26 (m, 2H), 7.23 (s, 1H), 7.05 (s, 1H), 6.98 (d, J=7.9 Hz, 1H), 4.96 (s, 1H), 4.82-4.67 (m, 2H), 4.60 (d, J=9.2 Hz, 1H), 4.55-4.51 (m, 1H), 4.50-4.39 (m, 2H), 4.37 (s, 1H), 4.31-4.24 (m, 2H), 4.20-4.17 (m, 2H), 3.81 (d, J=4.0 Hz, 4H), 3.47-3.43 (m, 2H), 3.40-3.32 (m, 1H), 2.99-2.93 (m, 2H), 2.66 (s, 3H), 2.47 (s, 3H), 2.17-2.05 (m, 3H), 1.98-1.89 (m, 1H), 1.72-1.58 (m, 4H), 1.42-1.28 (m, 4H), 1.24-1.19 (m, 2H), 0.96 (s, 9H)

### Example A341. 2-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-3-(2-hydroxyethoxy)-N-methylquinoline-6-carboxamide

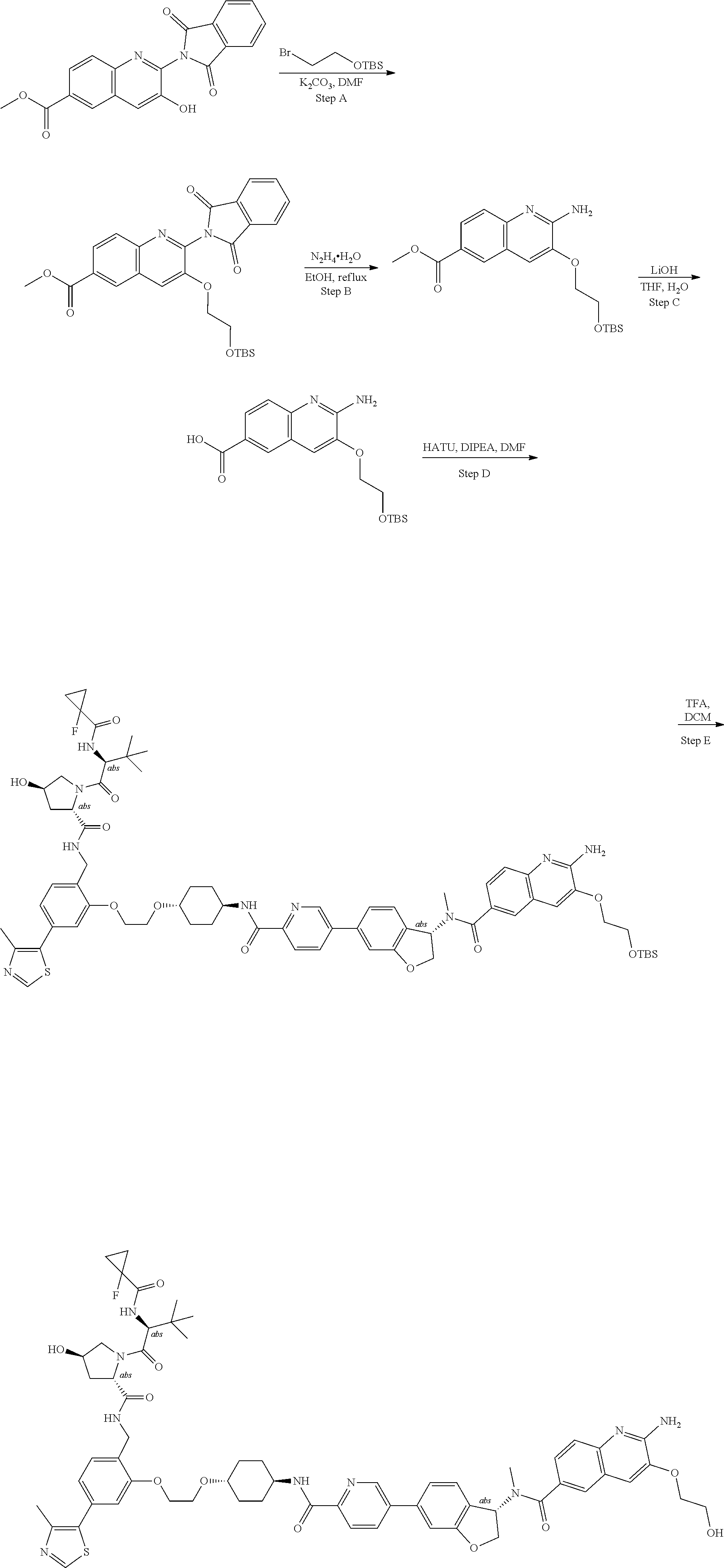

Example A341 was synthesized following the same procedures as described in Example A23 (12.0 mg, 5% yield over 5 steps) as a white solid. LC-MS (ESI): mass calcd. for C₆₁H₇₀FN₉O₁₁S, 1155.5/z found, 1156.3 [M+H]⁺. ¹H NMR (400 MHz, DMSO-d₆) δ 8.99 (s, 1H), 8.92 (s, 1H), 8.83 (s, 1H), 8.52-8.45 (m, 2H), 8.30-8.25 (m, 1H), 8.09 (d, J=8.2 Hz, 1H), 7.98 (s, 1H), 7.88 (s, 1H), 7.73 (s, 2H), 7.56 (d, J=7.7 Hz, 1H), 7.43-7.35 (m, 3H), 7.31-7.26 (m, 1H), 7.05 (s, 1H), 6.97 (d, J=7.8 Hz, 1H), 4.86-4.75 (m, 1H), 4.71-4.68 (m, 1H), 4.60 (d, J=9.1 Hz, 1H), 4.51 (t, J=8.2 Hz, 1H), 4.36 (s, 1H), 4.30 (d, J=6.0 Hz, 1H), 4.25-4.22 (m, 2H), 4.19-4.16 (m, 2H), 3.85-3.79 (m, 5H), 3.66-3.61 (m, 4H), 3.38-3.34 (m, 1H), 2.66 (s, 3H), 2.47 (s, 3H), 2.11-2.02 (m, 3H), 1.98-1.91 (m, 1H), 1.86 (d, J=9.8 Hz, 2H), 1.57-1.47 (m, 2H), 1.40-1.29 (m, 4H), 1.24-1.21 (m, 2H), 0.96 (s, 9H).

### Example A342. 2-amino-3-ethynyl-N-((3S)-6-(6-(3-((2′-(((2S,4R)-1-((S)-2-(1-fluorocyclopropane-1-carboxamido)-3,3-dimethylbutanoyl)-4-hydroxypyrrolidine-2-carboxamido)methyl)-5′-(4-methylthiazol-5-yl)-1,2,3,6-tetrahydro-[1,1′-biphenyl]-4-yl)methyl)-3,8-diazabicyclo[3.2.1]octane-8-carbonyl)pyridin-3-yl)-2,3-dihydrobenzofuran-3-yl)-N-methylquinoline-6-carboxamide

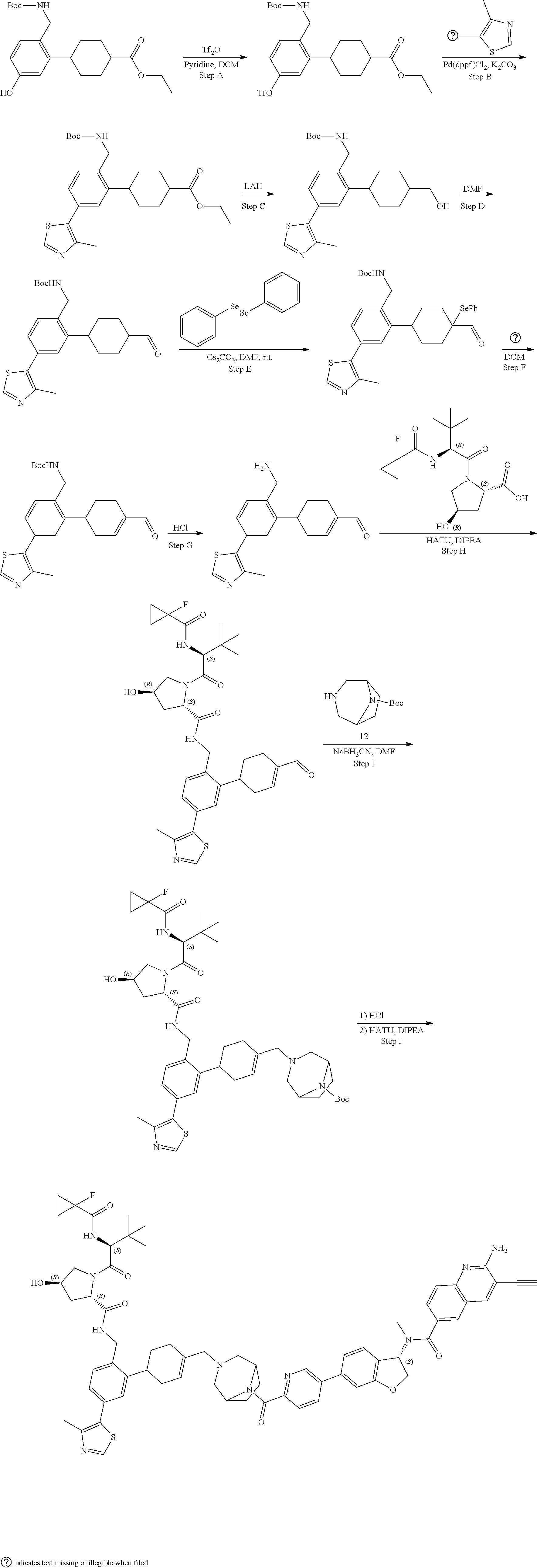

Step A: ethyl 4-(2-(((tert-butoxycarbonyl)amino)methyl)-5-(((trifluoromethyl)sulfonyl)oxy)phenyl)cyclohexane-1-carboxylate

A mixture of ethyl 4-(2-(((tert-butoxycarbonyl)amino)methyl)-5-hydroxyphenyl)cyclohexane-1-carboxylate (2.0 g, 5.30 mmol, 1.0 eq.), 1,1,1-trifluoro-N-phenyl-N-((trifluoromethyl)sulfonyl) methanesulfonamide (4.7 g, 13.2 mmol, 2.5 eq.), and TEA (1.6 g, 15.9 mmol, 3.0 eq.) in DCM (30 mL) was stirred at 30° C. for 2 hours under nitrogen. The resulting solution was diluted with 60 mL of water and extracted with EtOAc (50 mL×3). The combined organic layers were washed with brine (50 mL), dried over Na₂SO₄ and concentrated under reduced to afford pressure ethyl 4-(2-(((tert-butoxycarbonyl)amino)methyl)-5-(((trifluoromethyl)sulfonyl)oxy)phenyl)cyclohexane-1-carboxylate (2.0 g, 63% yield) as a yellow oil. LC-MS (ESI): mass calcd. For C₂₂H₃₀F₃NO₇S, 509.17; m/z found, 509.9 [M+H]⁺.

Step B: ethyl 4-(2-(((tert-butoxycarbonyl)amino)methyl)-5-(4-methylthiazol-5-yl)phenyl)cyclohexane-1-carboxylate

To a mixture of ethyl 4-(2-(((tert-butoxycarbonyl)amino)methyl)-5-(((trifluoromethyl)sulfonyl)oxy)phenyl)cyclohexane-1-carboxylate (2.0 g, 3.93 mmol, 1.0 eq.) in 1,4-Dioxane (20 mL)/Water (2 mL) was added 4-methyl-5-(4,4,5,5-tetramethyl-1,3,2-dioxaborolan-2-yl) thiazole (1.1 g, 4.71 mmol, 1.2 eq.), K₂CO₃ (1.4 g, 9.81 mmol, 2.5 eq.) and PdCl₂ (dppf)(287 mg, 393 μmol, 0.1 eq.). The reaction mixture was stirred at 100° C. for 3 hours under N₂. The resulting mixture was filtered and concentrated. The crude product was purified by column chromatography (silica gel, 0% EtOAc in PE~70% EtOAc in PE) to afford ethyl 4-(2-(((tert-butoxycarbonyl)amino)methyl)-5-(4-methylthiazol-5-yl)phenyl)cyclohexane-1-carboxylate (1.3 g, 65% yield) as a colorless oil. LC-MS (ESI): mass calcd. For C₂₅H₃₄N₂O₄S, 458.22; m/z found, 459.2 [M+H]⁺.

Step C: tert-butyl(2-(4-(hydroxymethyl)cyclohexyl)-4-(4-methylthiazol-5-yl)benzyl)carbamate

To a mixture of ethyl 4-(2-(((tert-butoxycarbonyl)amino)methyl)-5-(4-methylthiazol-5-yl)phenyl)cyclohexane-1-carboxylate (600 mg, 1.31 mmol, 1.0 eq.) in THF (10 mL) was added LAH (149 mg, 3.92 mmol, 3.0 eq.) at 0° C. The reaction mixture was stirred at 0° C. for 2 hours. The mixture was quenched with Potassium sodium tartrate, EtOAc (70 mL) was added followed by Celite, filtered and concentrated to afford the tert-butyl(2-(4-(hydroxymethyl)cyclohexyl)-4-(4-methylthiazol-5-yl)benzyl)carbamate (500 mg, 92% yield) as a white solid. LC-MS (ESI): mass calcd. For C₂₃H32N₂O₃S, 416.21; m/z found, 417.2 [M+H]⁺.

Step D: tert-butyl(2-(4-formylcyclohexyl)-4-(4-methylthiazol-5-yl)benzyl)carbamate

To a solution of tert-butyl(2-(4-(hydroxymethyl)cyclohexyl)-4-(4-methylthiazol-5-yl)benzyl)carbamate (500 mg, 1.20 mmol, 1.0 eq.) in DCM (10 mL) was added DMP (509 mg, 1.20 mmol, 1.0 eq.) and stirred at 0° C. for 2 hours. The resulting solution was filtered to give the crude tert-butyl(2-(4-formylcyclohexyl)-4-(4-methylthiazol-5-yl)benzyl)carbamate (500 mg, 99% yield) as a yellow oil. LC-MS (ESI): mass calcd. For C29H37FN406S, 588.24; m/z found, 589.3 [M+H]⁺.

Step E: tert-butyl(2-(4-formyl-4-(phenylselanyl)cyclohexyl)-4-(4-methylthiazol-5-yl)benzyl)carbamate

To a solution of tert-butyl(2-(4-formylcyclohexyl)-4-(4-methylthiazol-5-yl)benzyl)carbamate (500 mg, 1.21 mmol, 1.0 eq.) in DMF (8 mL) was added Cs₂CO₃ (1.2 g, 3.62 mmol, 3.0 eq.) and 1,2-diphenyldiselane (753 mg, 2.41 mmol, 2.0 eq.). The reaction was stirred at 30° C. for 4 hours. The resulting solution was diluted with 50 mL of water and extracted with EtOAc (30 mL×3). The combined organic layers were washed with brine (50 mL), dried over Na₂SO₄ and concentrated under reduced pressure to give tert-butyl(2-(4-formyl-4-(phenylselanyl)cyclohexyl)-4-(4-methylthiazol-5-yl)benzyl)carbamate (600 mg, 87% yield) as yellow oil. LC-MS (ESI): mass calcd. For C29H₃₄N₂O₃SSe, 570.15; m/z found, 571.0 [M+H]⁺.

Step F: tert-butyl ((4′-formyl-5-(4-methylthiazol-5-yl)-1′,2′,3′,6′-tetrahydro-[1,1′-biphenyl]-2-yl)methyl)carbamate

To a solution of tert-butyl(2-(4-formyl-4-(phenylselanyl)cyclohexyl)-4-(4-methylthiazol-5-yl)benzyl)carbamate (100 mg, 176 μmol, 1.0 eq.) in DCM (5 mL) was added H₂O₂ (299 mg, 269 μL, 30% Wt, 2.63 mmol, 15.0 eq.) at 0° C. and the mixture was stirred for 2 hours. The mixture was concentrated to afford the tert-butyl ((4′-formyl-5-(4-methylthiazol-5-yl)-1′,2′,3′,6′-tetrahydro-[1,1′-biphenyl]-2-yl)methyl)carbamate (200 mg, 55% yield) as a yellow solid. LC-MS (ESI): mass calcd. For C₂₃H₂₈N₂O₃S, 412.18; m/z found, 413.4 [M+H]⁺.

Steps G-J: 2-amino-3-ethynyl-N-(3S)-6-16-(3-(2′-(((25, 4R)-1-(S)-2-(1-fluorocyclopropane-1-carboxamido)-3,3-dimethylbutanoyl)-4-hydroxypyrrolidine-2-carboxamido)methyl)-5′-(4-methylthiazol-5-yl)-1,2,3,6-tetrahydro-[1,1′-biphenyl]-4-yl)methyl)-3,8-diazabicyclo[3.2.1]octane-8-carbonyl)pyridin-3-yl)-2,3-dihydrobenzofuran-3-yl)-N-methylquinoline-6-carboxamide

Example A342 was synthesized following the same procedures as described in Example A145 (12.1 mg, 3% yield over 4 steps) as a white solid. LC-MS (ESI): mass calcd. for C₆₆H₇₁FN₁₀O₇S, 1166.52; m/z found, 1167.8 [M+H]⁺. ¹H NMR (400 MHz, DMSO-d₆) δ 9.38 (s, 1H), 8.99 (d, J=6.9 Hz, 2H), 8.66 (s, 1H), 8.57-8.52 (m, 1H), 8.31 (d, J=8.6 Hz, 1H), 8.06 (s, 1H), 7.97 (d, J=7.9 Hz, 1H), 7.89 (d, J=8.2 Hz, 1H), 7.74 (s, 1H), 7.57 (d, J=7.7 Hz, 1H), 7.42 (d, J=10.1 Hz, 2H), 7.37 (s, 1H), 7.31 (s, 1H), 7.27 (d, J=7.6 Hz, 2H), 6.08 (s, 1H), 5.23 (s, 1H), 4.94 (s, 1H), 4.90 (s, 1H), 4.70 (s, 2H), 4.61-4.56 (m, 2H), 4.52-4.45 (m, 3H), 4.35 (s, 3H), 3.79 (s, 2H), 3.62 (s, 2H), 3.50-3.24 (m, 4H), 3.03 (s, 1H), 2.67 (s, 3H), 2.45 (s, 3H), 2.20 (s, 2H), 2.11-2.03 (m, 4H), 1.90-1.76 (m, 3H), 1.41-1.32 (m, 2H), 1.28-1.20 (m, 3H), 0.98-0.93 (m, 9H).

### Example A343. 2-amino-3-ethynyl-N-((S)-6-(6-(((1S,4S)-4-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl) propoxy)cyclohexyl) carbamoyl)pyridin-3-yl)-2,3-dihydrobenzofuran-3-yl)-N-methylquinoline-6-carboxamide

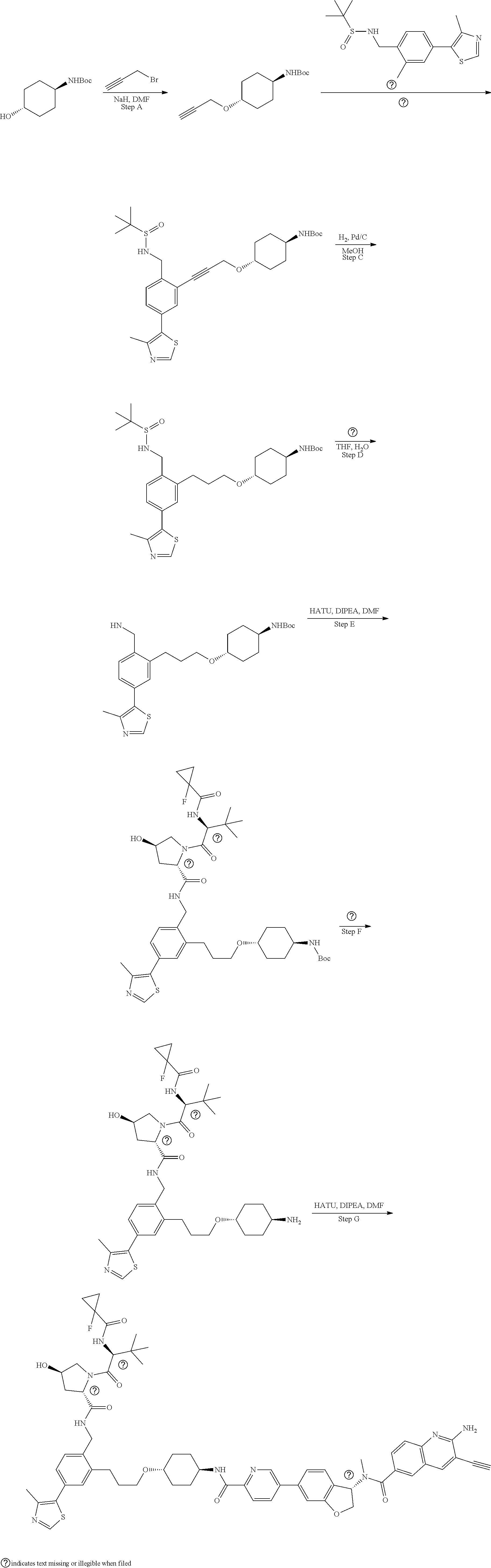

Step A: tert-butyl ((1r,4r)-4-(prop-2-yn-1-yloxy)cyclohexyl)carbamate

To a solution of tert-butyl ((1r,4r)-4-hydroxycyclohexyl)carbamate (5 g,23.2 mmol, 1 eq.) in DMF (40 mL) was added NaH (669 mg, 27.9 mmol, 1.2 eq.) at 0° C. under nitrogen and the mixture was stirred at 0° C. for 30 mins. A solution of 3-bromoprop-1-yne (2.9 g, 24.4 mmol, 1.05 eq.) in DMF (5 mL) was added to the mixture. The reaction mixture was stirred at room temperature for 3 hours. The reaction mixture was quenched with NH₄Cl (aq. 100 mL), and then extracted with EtOAc (300 mL×2). The combined organic layers were washed with brine three times, dried over anhydrous Na₂SO₄ and concentrated. The residue was purified by column chromatography (silica gel, 0% EtOAc in PE~20% EtOAc in PE) to afford tert-butyl ((1r,4r)-4-(prop-2-yn-1-yloxy)cyclohexyl)carbamate (2.5 g, 42% yield) as a yellow solid. LC-MS (ESI): mass calcd. For C14H₂₃NO_{3, 253.2}; m/z found, 198.2 [M+H−56]⁺.

Step B: tert-butyl ((Ir, 4r)-4-((3-(2-(((tert-butylsulfinyl)amino)methyl)-5-(4-methylthiazol-5-yl)phenyl) prop-2-yn-1-yl)oxy)cyclohexyl)carbamate

To a solution of 2-(((tert-butylsulfinyl)amino)methyl)-5-(4-methylthiazol-5-yl) phenyl trifluoromethanesulfonate (700 mg, 1.53 mmol, 1.0 eq.), tert-butyl ((1r,4r)-4-(prop-2-yn-1-yloxy)cyclohexyl)carbamate (427 mg, 1.69 mmol, 1.1 eq.), CuI (29 mg, 153 μmol, 0.1 eq.) in DMF (20 mL) was added Pd(PPh₃)₂Cl₂ (108 mg, 153 μmol, 0.1 eq.) and TEA (2.1 mL, 15.3 mmol, 10.0 eq.) at room temperature. The mixture was stirred at 80° C. for 16 hours under N₂ atmosphere. The resulting solution was diluted with 200 mL of H₂O and extracted with BtOAc (100 mL×3). The reaction mixture was quenched with water, and then extracted with EtOAc (100 mL×2). The combined organic layers were washed with brine three times, dried over anhydrous Na₂SO₄ and concentrated. The residue was purified by column chromatography (silica gel, 0% EtOAc in PE~100% EtOAc in PE) to afford tert-butyl ((1r,4r)-4-((3-(2-(((tert-butylsulfinyl)amino)methyl)-5-(4-methylthiazol-5-yl)phenyl) prop-2-yn-1-yl)oxy)cyclohexyl)carbamate (500 mg, 58% yield) as colorless oil. LC-MS (ESI): mass calcd. For C29H41N304S2, 559.3; m/z found, 560.3 [M+H]⁺.

Step C: tert-butyl ((1r,4r)-4-(3-(2-(((tert-butylsulfinyl)amino)methyl)-5-(4-methylthiazol-5-yl)phenyl) propoxy)cyclohexyl)carbamate

To a solution of tert-butyl ((1r,4r)-4-((3-(2-(((tert-butylsulfinyl)amino)methyl)-5-(4-methylthiazol-5-yl)phenyl) prop-2-yn-1-yl)oxy)cyclohexyl)carbamate (200 mg, 358 μmol, 1 eq.) in MeOH (10 mL) was added Pd/C (50 mg, 940 μmol, 2.6 eq.) and Pd (OH) 2/C (25 mg, 356 μmol, 1.0 eq.). The mixture was degassed with H₂ for 3 times and stirred at 50° C. for 16 hours. The reaction mixture was filtered and concentrated. The residue was purified by flash chromatography (100-200 mesh silica gel, 0%-100% EtOAc in PE) to afford tert-butyl ((1r,4r)-4-(3-(2-(((tert-butylsulfinyl)amino)methyl)-5-(4-methylthiazol-5-yl)phenyl) propoxy)cyclohexyl)carbamate (100 mg, 47% yield) as a yellow solid. LC-MS (ESI): mass calcd. For C₂₉H₄₅N₃O₄S₂, 563.3; m/z found, 564.3 [M+H]⁺.

Step D: tert-butyl ((1r,4r)-4-(3-(2-(aminomethyl)-5-(4-methylthiazol-5-yl)phenyl) propoxy)cyclohexyl)carbamate

To the solution of tert-butyl ((1r,4r)-4-(3-(2-(((tert-butylsulfinyl)amino)methyl)-5-(4-methylthiazol-5-yl)phenyl) propoxy)cyclohexyl)carbamate (90 mg, 160 μmol, 1.0 eq.) in THF (5 mL) and Water (1 mL) were added I₂ (81 mg, 639 μmol 2.0 eq.) and stirred at 50° C. for 2 hours. The reaction solution was used for next step directly. LC-MS (ESI): mass calcd. For C₂₅H₃₇N303S, 459.3; m/z found, 460.3 [M+H]⁺.

Steps E G: 2-amino-3-ethynyl-N-((S)-6-(6-(((1S,4S)-4-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl) propoxy)cyclohexyl)carbamoyl)pyridin-3-yl)-2,3-dihydrobenzofuran-3-yl)-N-methylquinoline-6-carboxamide

Example A343 was synthesized following the same procedures as described in Example A23 (3.2 mg, 3% yield over 3 steps) as a white solid. LC-MS (ESI): mass calcd, for C₆₂H₆₈FN₉O₈S, 1117.49; m/z found, 1118.5 [M+H]⁺. 1HI NMR (400 MHz, DMSO-d₆) δ 8.99 (s, 1H), 8.92 (s, 1H), 8.70-8.59 (m, 1H), 8.54-8.45 (m, 2H), 8.27 (d, J=10.1 Hz, 1H), 8.10-8.01 (m, 2H), 7.88 (d, J=8.7 Hz, 1H), 7.78-7.67 (m, 1H), 7.56 (d, J=7.7 Hz, 1H), 7.45-7.32 (m, 3H), 7.30-7.23 (m, 3H), 4.93 (s, 1H), 4.72-4.64 (m, 1H), 4.60 (d, J=9.2 Hz, 1H), 4.49 (t, J=8.2 Hz, 1H), 4.42-4.33 (m, 3H), 4.32-4.25 (m, 2H), 3.85-3.76 (m, 1H), 3.69-3.55 (m, 2H), 3.50-3.42 (m, 2H), 3.29-3.16 (m, 1H), 2.67 (s, 3H), 2.46 (s, 3H), 2.13-1.93 (m, 4H), 1.91-1.70 (m, 4H), 1.54-1.42 (m, 2H), 1.41-1.18 (m, 6H), 0.96 (s, 9H).

### Example A344. 2-amino-3-ethynyl-N-((S)-6-(3-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)amino)-1H-indazol-6-yl)-2,3-dihydrobenzofuran-3-yl)-N-methylquinoline-6-carboxamide

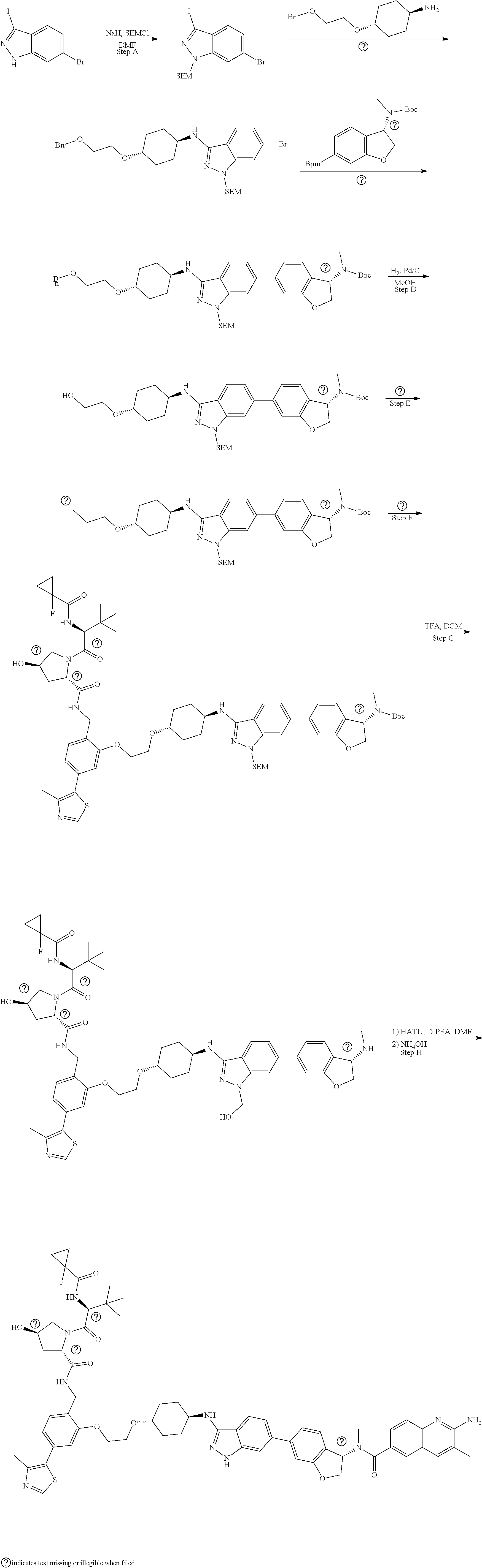

Example A344 was synthesized following the same procedures as described in Example A29 (0.8 mg, 6% yield over 9 steps) as a white solid. LC-MS (ESI): mass calcd. for C₆₂H₆₇FN1008S, 1130.48/z found, 1131.8 [M+H]⁺. ¹H NMR (400 MHz, DMSO-d₆) δ 11.42 (s, 1H), 8.99 (s, 1H), 8.53-8.47 (m, 1H), 8.30 (s, 1H), 7.87 (s, 1H), 7.78 (d, J=8.5 Hz, 1H), 7.67-7.64 (m, 1H), 7.55-7.50 (m, 1H), 7.47 (d, J=7.9 Hz, 1H), 7.43-7.40 (m, 2H), 7.31-7.27 (m, 2H), 7.19-7.17 (m, 2H), 7.06 (s, 1H), 6.97 (d, J=7.8 Hz, 1H), 6.74-6.64 (m, 2H), 5.77-5.74 (m, 1H), 5.17 (d, J=3.6 Hz, 1H), 4.78-4.62 (m, 3H), 4.59 (d, J=9.2 Hz, 1H), 4.54-4.49 (m, 1H), 4.37-4.29 (m, 2H), 4.25-4.17 (m, 3H), 3.84-3.79 (m, 2H), 3.65-3.61 (m, 1H), 3.53-3.38 (m, 2H), 3.30-3.30 (m, 2H), 2.68 (s, 3H), 2.47 (s, 3H), 2.16-1.96 (m, 9H), 1.39-1.27 (m, 5H), 0.95 (s, 9H).

### Example A345. 4-amino-N-((S)-6-(6-(((1S,4S)-4-(2-(2-(((2S,4S)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,1-dimethyl-1H-pyrazolo[4,3-c]quinoline-8-carboxamide

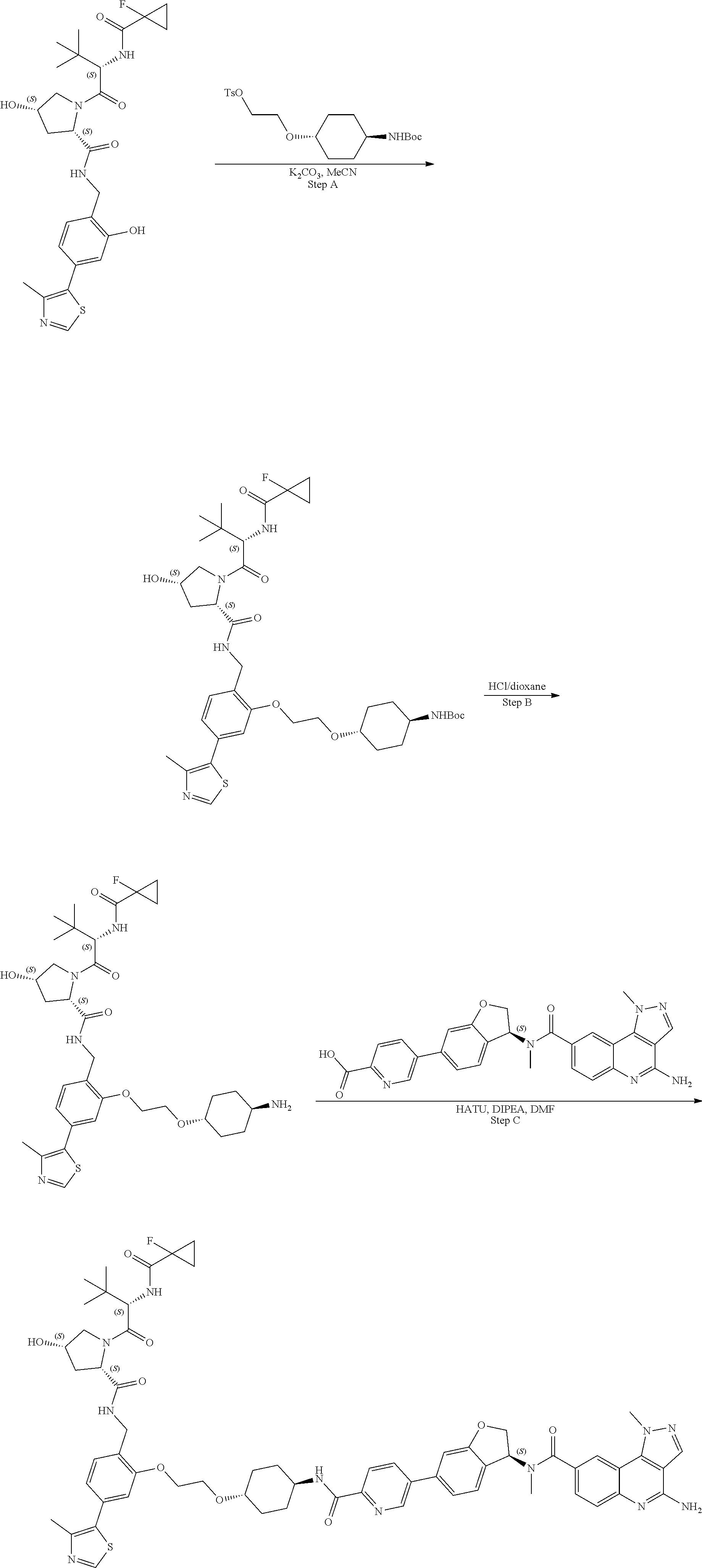

Example A345 was synthesized following the same procedures as described in Example A29 (2.7 mg, 8% yield over 3 steps) as a white solid. LC-MS (ESI): mass calcd. for C₆₁H₆₈FN₁₁O₉S, 1149.49; m/z found, 1150.3 [M+H]⁺. ¹H NMR (400 MHz, DMSO-d₆) δ 13.48 (s, 1H), 9.66 and 8.77 (s, 1H), 8.99 (s, 1H), 8.92 (s, 1H), 8.64 (s, 1H), 8.57 (t, J=5.8 Hz, 1H), 8.52-8.41 (m, 2H), 8.27 (d, J=7.2 Hz, 1H), 8.08 (d, J=8.3 Hz, 1H), 7.96-7.83 (m, 2H), 7.61 (s, 1H), 7.40 (d, J=7.8 Hz, 2H), 7.37-7.27 (m, 2H), 7.06 (s, 1H), 6.99-6.94 (m, 1H), 4.76 (s, 2H), 4.57-4.47 (m, 4H), 4.47-4.41 (m, 1H), 4.39-4.30 (m, 1H), 4.28-4.21 (m, 2H), 4.18 (s, 2H), 3.90-3.76 (m, 5H), 3.39-3.29 (m, 2H), 2.67 (s, 3H), 2.47 (s, 3H), 2.40-2.35 (m, 1H), 2.05 (d, J=9.9 Hz, 2H), 1.84 (s, 2H), 1.81-1.74 (m, 1H), 1.55-1.46 (m, 2H), 1.37-1.32 (m, 2H), 1.27-1.19 (m, 4H), 0.97 (s, 9H).

### Example A346. 2-amino-3-ethynyl-N-((S)-6-(6-(((1S,4S)-4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-2-methylpropyl)(methyl)amino) cyclohexyl)carbamoyl)pyridin-3-yl)-2,3-dihydrobenzofuran-3-yl)-N-methylquinoline-6-carboxamide

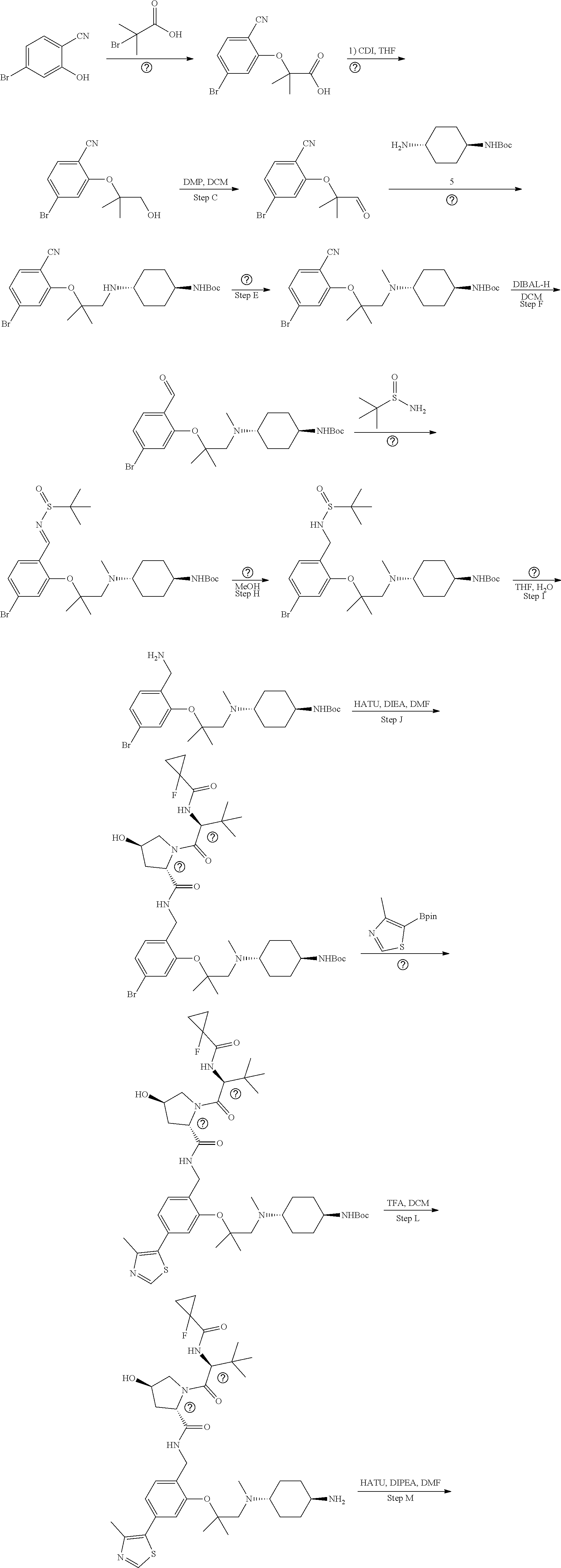

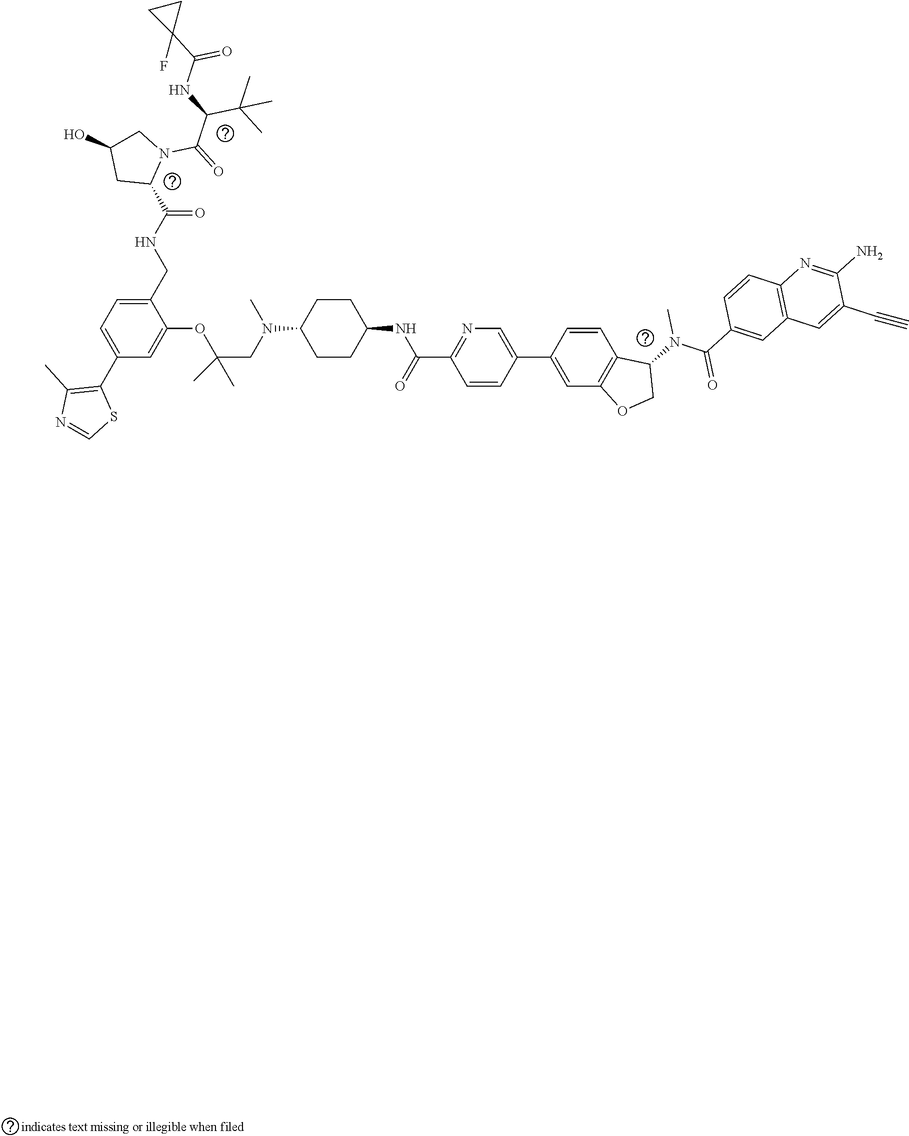 [SMILES: C#Cc1cc2cc(C(=O)N(C)[C@@H]3COc4cc(-c5ccc(C(=O)N[C@H]6CC[C@H](N(C)CC(C)(C)Oc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N]

Step A: 2-(5-bromo-2-cyanophenoxy)-2-methylpropanoic acid

To a solution of 4-bromo-2-hydroxybenzonitrile (10 g, 50.5 mmol, 1.0 eq.) in MeCN (150 mL) was added 2-bromo-2-methylpropanoic acid (10.1 g, 60.6 mmol, 1.2 eq.) and K₂CO₃ (20.9 g, 151 mmol, 3.0 eq.) at room temperature. The mixture was stirred at 70° C. for 12 hours under N₂ atmosphere. The reaction mixture was poured into water (200 mL) and then extracted with EtOAc (200 mL×2). The combined organic layers were washed with brine, dried over anhydrous Na₂SO₄ and concentrated. The residue was purified by column chromatography (100-200 mesh silica gel, 0-100% EtOAc in PE) to afford 2-(5-bromo-2-cyanophenoxy)-2-methylpropanoic acid (6.00 g, 41% yield) as a white solid. LC-MS (ESI): mass calcd. For C₁₁H₁₀BrNO₃, 282.98; m/z found, 284.0 [M+H]⁺.

Step B: 4-bromo-2-((1-hydroxy-2-methylpropan-2-yl)oxy)benzonitrile

To a solution of 2-(5-bromo-2-cyanophenoxy)-2-methylpropanoic acid (6 g, 21.1 mmol, 1.0 eq.) in THF (50 mL) was added CDI (4.1 g, 25.3 mmol, 1.2 eq.) and stirred at room temperature for 2 hours. Then added NaBH₄ (1,6 g, 42.2 mmol, 2.0 eq.) in THF (30 mL) and H₂O (15 mL) solution. The mixture was stirred at room temperature for 1 hour. The reaction mixture was poured into water (100 mL) and then extracted with EtOAc (200 mL×2). The combined organic layers were washed with brine, dried over anhydrous Na₂SO₄ and concentrated. The residue was purified by column chromatography (100-200 mesh silica gel, 0-100% EtOAc in PE) to afford 4-bromo-2-((1-hydroxy-2-methylpropan-2-yl)oxy)benzonitrile (4 g, 7% yield) as a white solid. LC-MS (ESI): mass calcd. For C₁₁H₁₂BrNO₂, 269.01; m/z found, 270.0 [M+H]⁺.

Step C: 4-bromo-2-((2-methyl-1-oxopropan-2-yl)oxy)benzonitrile

To a solution of 4-bromo-2-((1-hydroxy-2-methylpropan-2-yl)oxy)benzonitrile (2.5 g, 9.3 mmol, 1.0 eq.) in DCM (30 mL) was added Dess-Martin periodinane (5.9 g, 13.9 mmol, 1.5 eq.) at room temperature. The mixture was stirred at room temperature for 2 hours under N₂ atmosphere. The reaction mixture was poured into water (20 mL) and then extracted with EtOAc (20 mL×2). The combined organic layers were washed with brine, dried over anhydrous Na₂SO₄ and concentrated to afford crude 4-bromo-2-((2-methyl-1-oxopropan-2-yl)oxy)benzonitrile (1.5 g, 61% yield) as a solid. LCMS showed no MS signal.

Step D: tert-butyl ((1r,4r)-4-((2-(5-bromo-2-cyanophenoxy)-2-methylpropyl)amino)cyclohexyl)carbamate

To a stirred mixture of 4-bromo-2-((2-methyl-1-oxopropan-2-yl)oxy)benzonitrile (1.5 g, 5.6 mmol, 1.0 eq.) in DMF (20 mL) was added tert-butyl ((1r,4r)-4-aminocyclohexyl)carbamate (1.4 g, 6.7 mmol, 1.2 eq.), and AcOH (0.1 mL) and NaBH₃CN (704 mg, 11.2 mmol, 1.2 eq.) at room temperature. The reaction mixture was stirred at room temperature for 1 hour. The reaction mixture was poured into water (20 mL) and then extracted with EtOAc (20 mL×2). The combined organic layers were washed with brine, dried over anhydrous Na₂SO₄ and concentrated. The residue was purified by column chromatography (100-200 mesh silica gel, 0-100% EtOAc in

PE) to afford tert-butyl ((1r,4r)-4-((2-(5-bromo-2-cyanophenoxy)-2-methylpropyl)amino)cyclohexyl)carbamate (400 mg, 15% yield) as a white solid. LC-MS (ESI): mass calcd. For C₂₂H₃₂BrN₃O₃,465.16; m/z found, 466.2 [M+H]⁺.

Step E: tert-butyl ((1r,4r)-4-((2-(5-bromo-2-cyanophenoxy)-2-methylpropyl)(methyl)amino)cyclohexyl)carbamate

To a stirred mixture of tert-butyl ((1r,4r)-4-((2-(5-bromo-2-cyanophenoxy)-2-methylpropyl)amino)cyclohexyl)carbamate (400 mg, 0.9 mmol, 1.0 eq.) in MeOH (6 mL) was added Formaldehyde (47 μL, 1.8 mmol, 2.0 eq.) and NaBH₃CN (113 mg, 1.8 mmol, 2.0 eq.) at room temperature. The reaction mixture was stirred at room temperature for 1 hour. The reaction mixture was poured into water (20 mL) and then extracted with EtOAc (20 mL×2). The combined organic layers were washed with brine, dried over anhydrous Na₂SO₄ and concentrated. The residue was purified by column chromatography (100-200 mesh silica gel, 0-100% EtOAc in PE) to afford tert-butyl ((1r,4r)-4-((2-(5-bromo-2-cyanophenoxy)-2-methylpropyl)(methyl)amino)cyclohexyl)carbamate (175 mg, 42% yield) as a white solid. LC-MS (ESI): mass calcd. For C₂₃H₃₄BrN₃O₃, 479.18; m/z found, 480.2 [M+H]⁺.

Steps F-M: 2-amino-3-ethynyl-N-((S)-6-(6-(((1S, 4S)-4-((2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-2-methylpropyl)(methyl)amino) cyclohexyl)carbamoyl)pyridin-3-yl)-2,3-dihydrobenzofuran-3-yl)-N-methylquinoline-6-carboxamide

Example A346 was synthesized following the same procedures as described in Example A29 (1.0 mg, 0.8% yield over 8 steps) as a white solid. LC-MS (ESI): mass calcd. for C₆₄H₇₃FN₁₀O₈S, 1160.53; m/z found, 1163.0 [M+H]⁺. ¹H NMR (400 MHz, MeOD) δ 8.92 (s, 1H), 8.88 (s, 1H), 8.73-8.62 (m, 1H), 8.25-8.12 (m, 2H), 8.07 (s, 1H), 7.96 (d, J=8.7 Hz, 1H), 7.79 (s, 1H), 7.58 (d, J=7.4 Hz, 1H), 7.47 (d, J=8.9 Hz, 1H), 7.39-7.33 (m, 1H), 7.31-7.20 (m, 3H), 4.76-4.70 (m, 3H), 4.63-4.56 (m, 1H), 4.50 (s, 2H), 4.39 (s, 1H), 4.06-3.92 (m, 1H), 3.91-3.74 (m, 3H), 3.56-3.42 (m, 3H), 3.14 (s, 3H), 2.88-2.70 (m, 3H), 2.51 (s, 3H), 2.39-2.29 (m, 1H), 2.29-2.16 (m, 4H), 2.12-2.00 (m, 1H), 1.99-1.89 (m, 1H), 1.86-1.77 (m, 1H), 1.74-1.53 (m, 8H), 1.41-1.22 (m, 6H), 1.01 (d, J=4.3 Hz, 9H).

### Example A347. 2-amino-3-ethynyl-N-((S)-6-(6-(((1S,4S)-4-(2-((2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)amino)-2-oxoethoxy)cyclohexyl) carbamoyl)pyridin-3-yl)-2,3-dihydrobenzofuran-3-yl)-N-methylquinoline-6-carboxamide

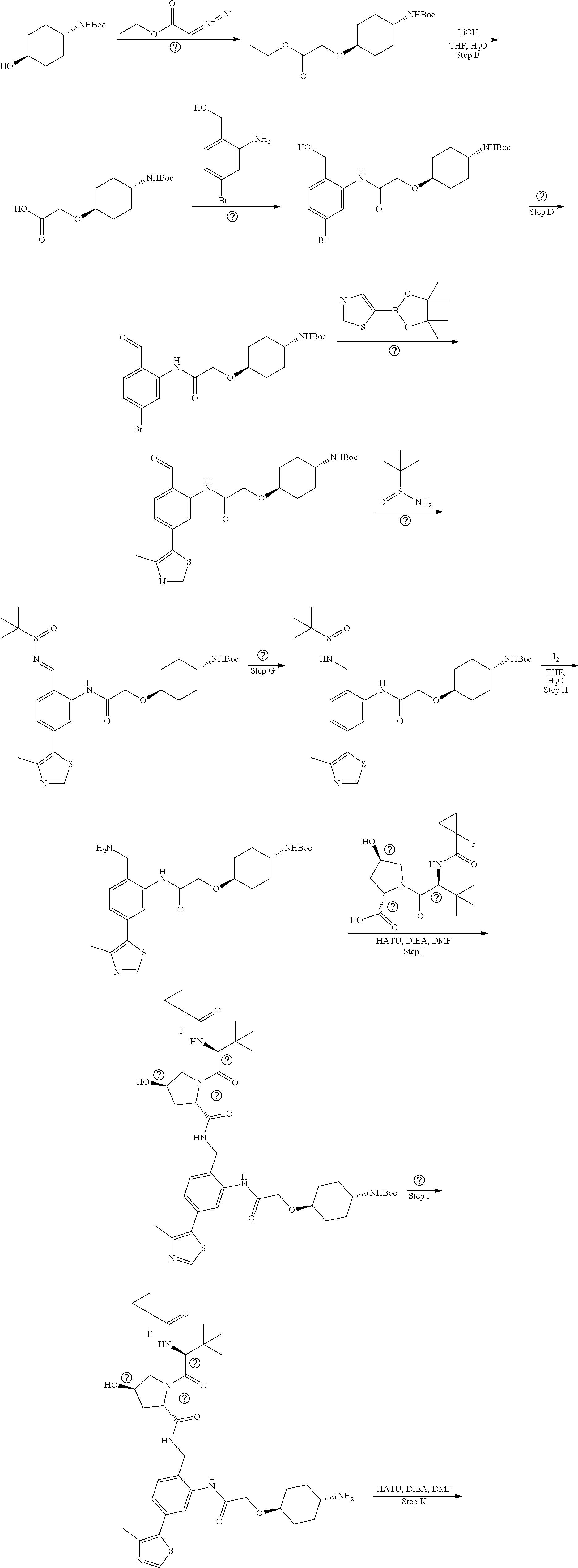

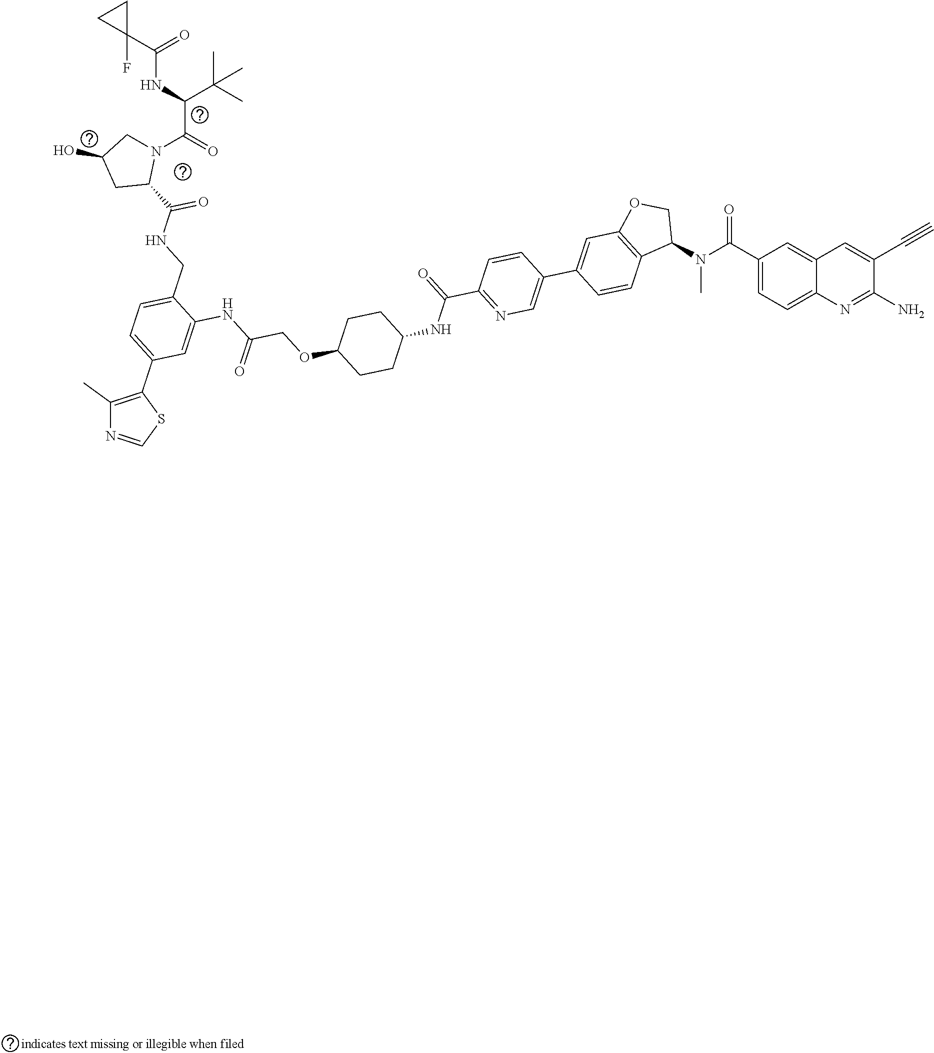 [SMILES: C#Cc1cc2cc(C(=O)N(C)[C@@H]3COc4cc(-c5ccc(C(=O)N[C@H]6CC[C@H](OCC(=O)Nc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)ccc2nc1N]

Step A: ethyl 2-(((1r, 4r)-4-((tert-butoxycarbonyl)amino)cyclohexyl)oxy)acetate

To the solution of tert-butyl ((1r,4r)-4-hydroxycyclohexyl)carbamate (5 g, 23.2 mmol, 1.0 eq.) in DCM (4.00 mL) were added Rhodium (II) AcetateDimer (513 mg, 1.2 mmol, 0.05 eq.) and ethyl 2-diazoacetate (8 g, 69.7 mmol, 3.0 eq.) at 0° C. and stirred for 15 hours at room temperature. LCMS showed this reaction was completed. The reaction was monitored by analysis of LC-MS. The resulting solution was quenched with Potassium sodium tartrate and extracted with EtOAc (3×30 mL). The combined organic layers were washed with brine, dried over anhydrous Na₂SO₄ and concentrated under reduced pressure. The residue was purified by silica gel cholumn chromatography (EA/PE=0~20%) to afford the ethyl 2-(((1r,4r)-4-((tert-butoxycarbonyl)amino)cyclohexyl)oxy)acetate (2.8 g, 40% yield) as a colorless oil. LC-MS (ESI): mass calcd. For C₁₅H₂₇NO₅, 301.19; m/z found, 302 [M+H]⁺.

Step B: methyl 4-bromo-2-(2-oxoethyl)benzoate

To the solution of ethyl 2-(((1r,4r)-4-((tert-butoxycarbonyl)amino)cyclohexyl)oxy)acetate (2.8 g, 9.3 mmol, 1.0 eq.) in THF (15 mL) was added lithium hydroxide (445 mg, 18.6 mmol, 2.0 eq.) in Water (15.0 mL) at 0° C. The mixture was stirred at room temperature for 2 hours. The resulting solution was acified and diluted with 30 mL of water and extracted with EtOAc (10 mL×3). The combined organic layers were washed with brine, dried over anhydrous Na₂SO₄ and concentrated under reduced pressure to afford 2-(((1r,4r)-4-((tert-butoxycarbonyl)amino)cyclohexyl)oxy) acetic acid (2.4 g, 94% yield) as a yellow solid. LC-MS (ESI): mass calcd. For C₁₃H₂₃NO₅, 273.16; m/z found, 274 [M+H]⁺.

Step C: tert-butyl ((1r,4r)-4-(2-((5-bromo-2-(hydroxymethyl)phenyl)amino)-2-oxoethoxy)cyclohexyl)carbamate

To a mixture of 2-(((1r,4r)-4-((tert-butoxycarbonyl)amino)cyclohexyl)oxy) acetic acid (2.4 g, 8.8 mmol, 1.0 eq.) and (2-amino-4-bromophenyl) methanol (1.8 g, 8.8 mmol, 1.0 eq.) in MeOH (15 mL) and DCM (15 mL) was added EEDQ (4.3 g, 17.6 mmol, 2.0 eq.) at 0° C. The mixture was stirred at 50° C. for 2 hours. The resulting solution was diluted with 30 mL of water and extracted with DCM (3×20 mL). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford tert-butyl ((1r,4r)-4-(2-((5-bromo-2-(hydroxymethyl)phenyl)amino)-2-oxoethoxy)cyclohexyl)carbamate (2 g, 50% yield) as a yellow solid. LC-MS (ESI): mass calcd. For C₂₀H₂₉BrN₂O₅, 456.13; m/z found, 457 [M+H]⁺.

Step D: tert-butyl ((1r,4r)-4-(2-((5-bromo-2-formylphenyl)amino)-2-oxoethoxy)cyclohexyl)carbamate

To the solution of tert-butyl ((1r,4r)-4-(2-((5-bromo-2-(hydroxymethyl)phenyl)amino)-2-oxoethoxy)cyclohexyl)carbamate (2 g, 4.4 mmol, 1.0 eq.) in DCM (4 mL) were added Manganese dioxide (3.8 g, 43.7 mmol, 10.0 eq.) and stirred for 15 hours at room temperature. The residue was filtered and purified by silica gel cholumn chromatography (MeOH/DCM=0~10%) to afford the tert-butyl ((1r,4r)-4-(2-((5-bromo-2-formylphenyl)amino)-2-oxoethoxy)cyclohexyl)carbamate (1.6 g, 80% yield) as a colorless solid. LC-MS (ESI): mass calcd. For C₂₀H₂₇BrN₂O₅, 454.11; m/z found, 455 [M+H]⁺.

Steps E-K: 2-amino-3-ethynyl-N-(S)-6-(6-((18,4S)-4-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)amino)-2-oxoethoxy)cyclohexyl) carbamoyl)pyridin-3-yl)-2,3-dihydrobenzofuran-3-yl)-N-methylquinoline-6-carboxamide

Example A347 was synthesized following the same procedures as described in Example A23 (7.1 mg, 2% yield over 7 steps) as a white solid. LC-MS (ESI): mass calcd, for C₆₁H₆₅FN₁₀O₉S, 1132.46; m/z found, 1134 [M+H]⁺. ¹H NMR (400 MHz, DMSO-d₆) δ 9.66 (s, 1H), 9.01 (s, 1H), 8.93 (s, 1H), 8.78-8.46 (m, 5H), 8.28 (d, J=8.0 Hz, 1H), 8.09 (d, J=8.7 Hz, 2H), 7.91 (d, J=8.5 Hz, 1H), 7.77-7.68 (m, 2H), 7.56 (d, J=7.8 Hz, 1H), 7.48 (d, J=8.0 Hz, 1H), 7.42-7.34 (m, 2H), 7.27 (d, J=9.1 Hz, 2H), 4.96 (s, 1H), 4.84-4.69 (m, 2H), 4.59 (d, J=9.2 Hz, 1H), 4.48 (t, J=8.2 Hz, 1H), 4.36 (s, 1H), 4.29-4.24 (m, 2H), 4.15 (s, 3H), 3.67-3.58 (m, 2H), 3.49-3.39 (m, 2H), 2.67 (s, 3H), 2.47 (s, 3H), 2.16-2.06 (m, 3H), 1.94-1.85 (m, 3H), 1.57-1.48 (m, 2H), 1.47-1.40 (m, 2H), 1.37-1.28 (m, 2H), 1.24-1.20 (m, 2H), 0.94 (s, 9H).

### Example A350. 2-amino-3-ethynyl-N-((S)-6-(6-(((18,4S)-4-((1-(2-(((2S,4R)-1-((S)-2-(1 fluorocyclopropane-1-carboxamido)-3,3-dimethylbutanoyl)-4-hydroxypyrrolidine-2-carboxamido)methyl)-5-(4-methylthiazol-5-yl)phenoxy)-2-methylpropan-2-yl)oxy)cyclohexyl)carbamoyl)pyridin-3-yl)-2,3-dihydrobenzofuran-3-yl)-N-methylquinoline-6-carboxamide

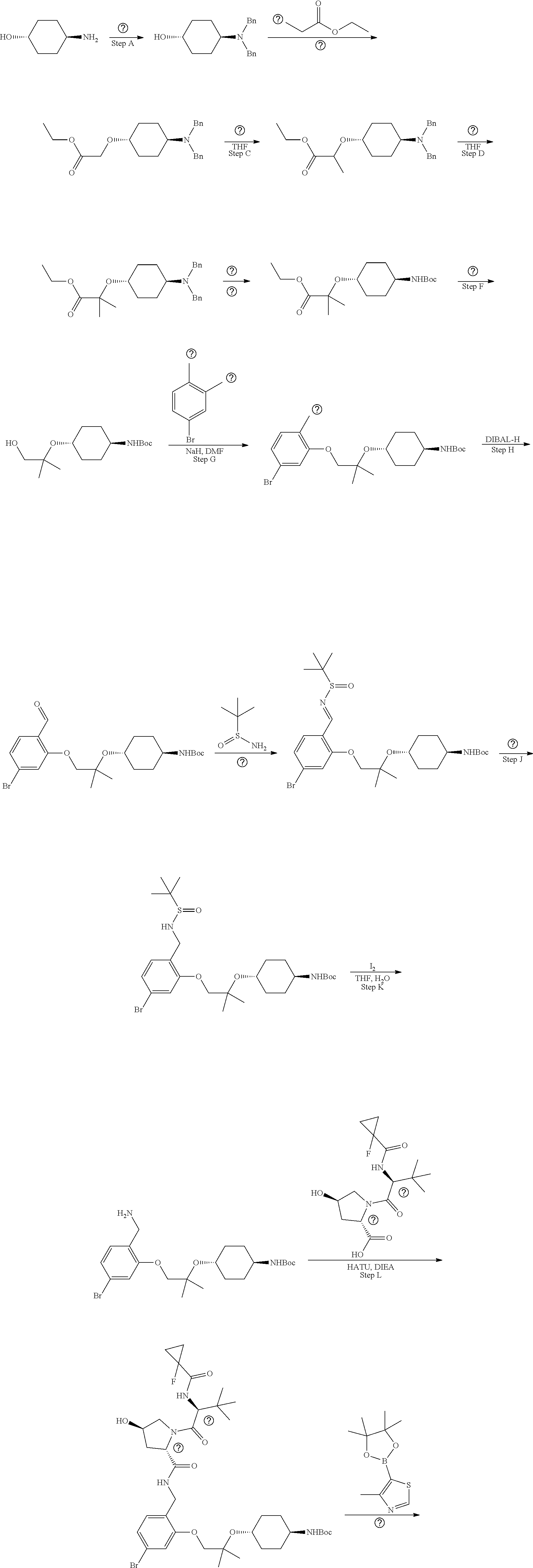

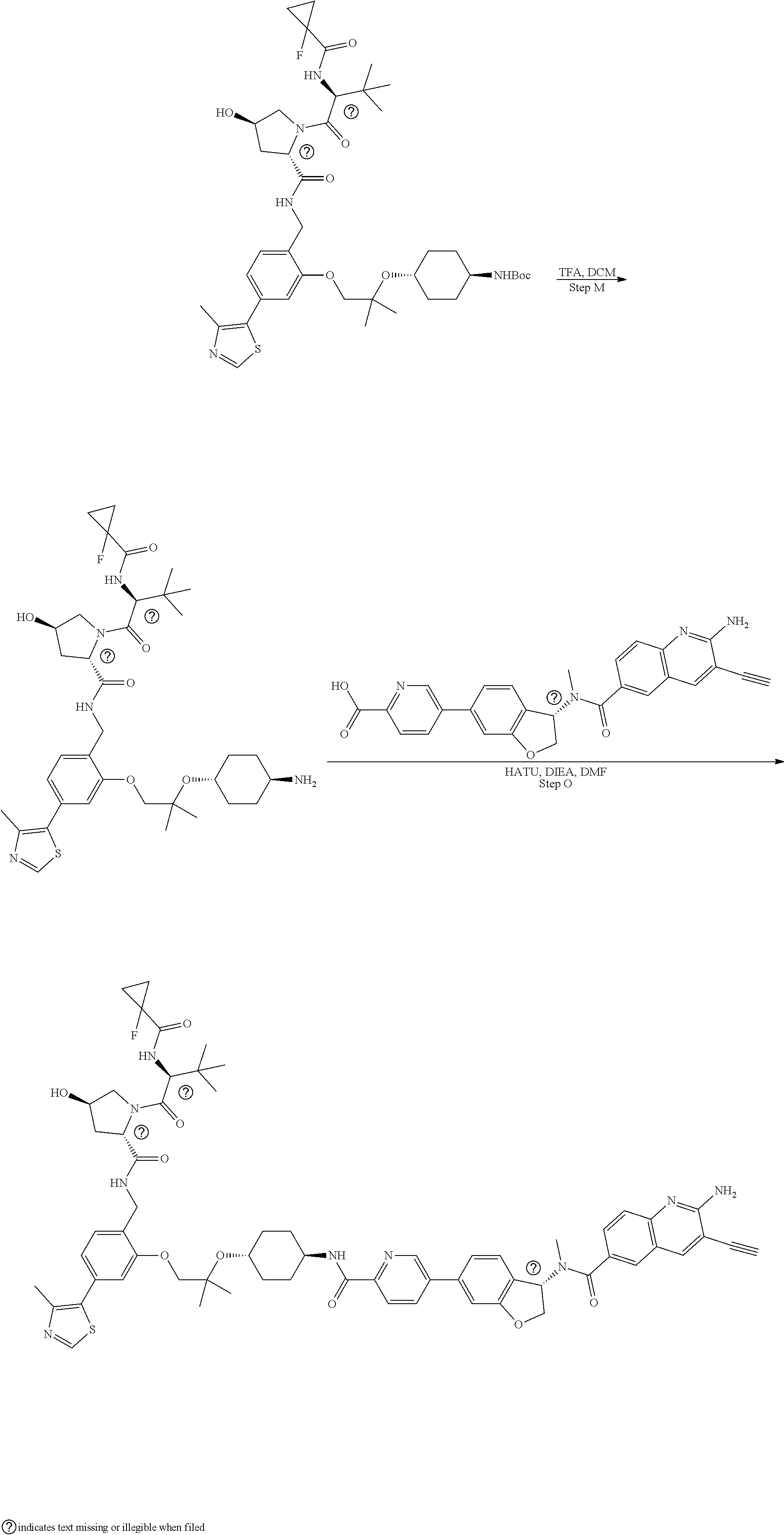

Step A: (1r,4r)-4-(dibenzylamino)cyclohexan-1-ol

To a solution of (1r,4r)-4-aminocyclohexan-1-ol (10 g, 86.8 mmol, 1.0 eq.) in MeCN (150 mL) was added K₂CO₃ (30 g, 217 mmol, 2.5 eq.) and Benzyl bromide (29.7 g, 174 mmol, 2.0 eq.) at room temperature. The mixture was then stirred at room temperature for 16 hours under N₂ atmosphere. The residue was diluted with water. The solid was collected by filtration. The residue was concentrated under reduced pressure to afford (1r,4r)-4-(dibenzylamino)cyclohexan-1-ol (20 g, 98% yield) as a white solid. LC-MS (ESI): mass calcd. For C20H₂₅NO, 295.2; m/z found, 296.2 [M+H]⁺.

Step B: ethyl 2-(((1r,4r)-4-(dibenzylamino)cyclohexyl)oxy)acetate

To a solution of (1r,4r)-4-(dibenzylamino)cyclohexan-1-ol (7.5 g, 25.4 mmol, 1.0 eq.) in DCM (75 mL) was added Rhodium (II) AcetateDimer (1.12 g, 2.54 mmol, 0.1 eq.) and ethyl 2-diazoacetate (8.69 g, 76.2 mmol, 3.0 eq.) at 0° C. The mixture was then stirred at room temperature for 16 hours under N₂ atmosphere. The resulting solution was diluted with 30 mL of H₂O and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~10% EtOAc in PE) to afford ethyl 2-(((1r,4r)-4-(dibenzylamino)cyclohexyl)oxy)acetate (6 g, 62% yield) as a yellow oil. LC-MS (ESI): mass calcd. For C₂₄H₃₁NO_{3, 381.2}; m/z found, 382.2 [M+H]⁺

Step C: ethyl 2-(((1r,4r)-4-(dibenzylamino)cyclohexyl)oxy) propanoate

To a solution of ethyl 2-(((1r,4r)-4-(dibenzylamino)cyclohexyl)oxy)acetate (2 g, 5.24 mmol, 1.0 eq.) in THF (20 mL) was added LiHMDS (1.75 g, 10.5 mmol, 2.0 eq.) at −65° C. The mixture was then stirred at −65° C. for 30 mins under N₂ atmosphere. Then Mel (7.44 g, 52.4 mmol, 10.0 eq.) was added at −65° C. The mixture was then stirred at room temperature for 16 hours under N2 atmosphere. The resulting solution was diluted with 30 mL of NH₄Cl/H₂O and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~5% EtOAc in PE) to afford ethyl 2-(((1r,4r)-4-(dibenzylamino)cyclohexyl)oxy) propanoate (190 mg, 9% yield) as a yellow oil. LC-MS (ESI): mass calcd. For C₂₅H₃₃NO_{3, 395.2}; m/z found, 396.5 [M+H]⁺.

Step D: ethyl 2-(((1r,4r)-4-(dibenzylamino)cyclohexyl)oxy)-2-methylpropanoate

To a solution of ethyl 2-(((1r,4r)-4-(dibenzylamino)cyclohexyl)oxy) propanoate (190 mg, 480 μmol, 1.0 eq.) in THF (5 mL) was added Lithium diisopropylamide solution (103 mg, 961 μmol, 2.0 eq.) at −65° C. The mixture was then stirred at −65° C. for 30 mins under N₂ atmosphere. Then Mel (205 mg, 1.44 mmol, 3.0 eq.) and HMPA (172 mg, 961 μmol, 2.0 eq.) was added at −65° C. The mixture was then stirred at room temperature for 1 hour under N₂ atmosphere. The resulting solution was diluted with 30 mL of NH₄Cl/H₂O and extracted with EtOAc (30 mL×2).

The combined organic layers were washed with brine, dried over anhydrous Na₂SO₄ and concentrated under reduced pressure. The residue was purified by column chromatography (silica gel, 0% EtOAc in PE~5% EtOAc in PE) to afford ethyl 2-(((1r,4r)-4-(dibenzylamino)cyclohexyl)oxy)-2-methylpropanoate (140 mg, 71% yield) as a white solid. LC-MS (ESI): mass calcd. For C₂₆H₃₅NO₃, 409.3; m/z found, 410.5 [M+H]⁺.

Step E: ethyl 2-(((Ir, 4r)-4-((tert-butoxycarbonyl)amino)cyclohexyl)oxy)-2-methylpropanoate

To a solution of ethyl 2-(((1r,4r)-4-(dibenzylamino)cyclohexyl)oxy)-2-methylpropanoate (710 mg, 1.73 mmol, 1.0 eq.) in MeOH (10 mL) was added Pd/C (355 mg, 3.34 mmol, 1.9 eq.), Palladium (2+) dihydroxide (355 mg, 2.53 mmol, 1.5 eq.) and Di-tert-butyl dicarbonate (757 mg, 3.47 mmol, 2.0 eq.) at room temperature. The mixture was then stirred at room temperature for 1 hour under H₂ atmosphere. The solid was filtered out. The resulting solution was concentrated under reduced pressure. The residue was purified by column chromatography (silica gel, 0% EtOAc in PE~15% EtOAc in PE) to afford ethyl 2-(((1r,4r)-4-((tert-butoxycarbonyl)amino)cyclohexyl)oxy)-2-methylpropanoate (300 mg, 52% yield) as a yellow oil.

LC-MS (ESI): mass calcd. For C₁₇H₃₁NO_{5, 329.2}; m/z found, 330.1 [M+H]⁺.

Step F: tert-butyl ((1r,4r)-4-((1-hydroxy-2-methylpropan-2-yl)oxy)cyclohexyl)carbamate

To a solution of ethyl 2-(((1r,4r)-4-((tert-butoxycarbonyl)amino)cyclohexyl)oxy)-2-methylpropanoate (417 mg, 1.27 mmol, 1.0 eq.) in THE (5 mL) was added LiAlH₄ (96 mg, 2.53 mmol, 2.0 eq.) at 0° C. The mixture was then stirred at room temperature for 1 hour under N₂ atmosphere. The solid was filtered out. The residue was concentrated under reduced pressure to afford tert-butyl ((1r,4r)-4-((1-hydroxy-2-methylpropan-2-yl)oxy)cyclohexyl)carbamate (350 mg, 96% yield) as a yellow oil. LC-MS (ESI): mass calcd. For C₁₅H29NO_{4, 287.2}; m/z found, 288.2 [M+H]⁺.

Steps G-O: 2-amino-3-ethynyl-N-(S)-6-(6-(((1S,4S)-4-(1-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)-2-methylpropan-2-yl)oxy)cyclohexyl)carbamoyl)pyridin-3-yl)-2,3-dihydrobenzofuran-3-yl)-N-methylquinoline-6-carboxamide

Example A350 was synthesized following the same procedures as described in Example A29 (5.5 mg, 1% yield over 9 steps) as a white solid. LC-MS (ESI): mass calcd, for C₆₃H₇₀FN₉O₉S, 1147.5/z found, 1148.0 [M+H]⁺. ¹H NMR (400 MHz, DMSO-d₆) δ 8.99 (s, 1H), 8.92 (s, 1H), 8.62 (s, 1H), 8.57-8.48 (m, 1H), 8.42 (d, J=8.3 Hz, 1H), 8.27 (d, J=8.1 Hz, 2H), 8.10-8.00 (m, 2H), 7.87 (d, J=8.8 Hz, 1H), 7.73 (s, 1H), 7.56 (d, J=7.8 Hz, 1H), 7.43 (d, J=7.8 Hz, 1H), 7.39 (d, J=7.7 Hz, 1H), 7.34 (s, 1H), 7.30 (d, J=9.0 Hz, 1H), 7.03 (s, 1H), 6.96 (d, J=7.7 Hz, 1H), 4.91 (s, 1H), 4.71-4.67 (m, 1H), 4.62-4.58 (m, 1H), 4.56-4.51 (m, 1H), 4.39-4.21 (m, 4H), 3.91-3.89 (m, 2H), 3.68-3.67 (m, 1H), 3.66-3.65 (m, 1H), 3.63-3.61 (m, 2H), 3.60-3.59 (m, 1H), 2.66 (s, 3H), 2.46 (s, 3H), 2.15-2.06 (m, 1H), 1.99-1.92 (m, 1H), 1.87-1.74 (m, 4H), 1.57-1.47 (m, 2H), 1.44-1.32 (m, 4H), 1.29 (s, 6H), 1.27-1.11 (m, 4H), 0.96 (s, 9H).

### Example A352. 2-amino-3-ethynyl-N-((S)-6-(6-(((1S,4S)-4-(2-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenoxy)ethoxy)cyclohexyl)carbamoyl)

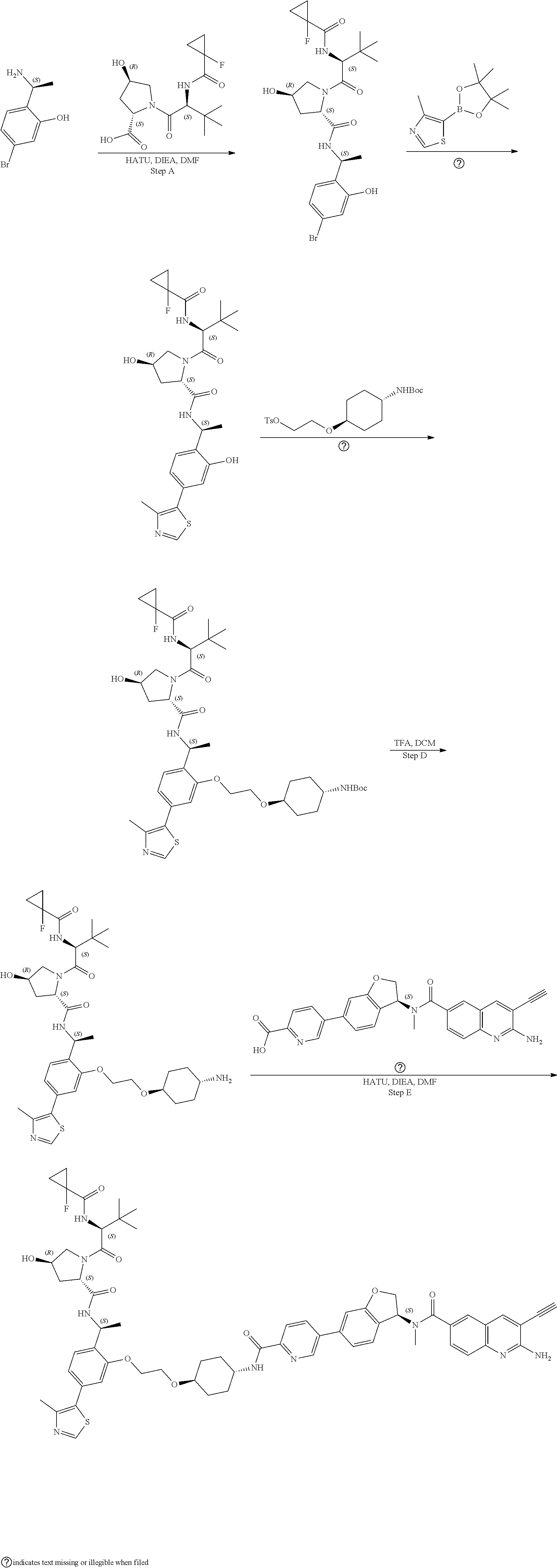

Example A352 was synthesized following the same procedures as described in Example A29 (9.4 mg, 2% yield over 5 steps) as a white solid. LC-MS (ESI): mass calcd, for C₆₂H₆₈FN₉O₉S, 1133.5/z found, 1134.9 [M+H]⁺. ¹H NMR (400 MHz, DMSO-d₆) δ 8.99 (s, 1H), 8.93 (s, 1H), 8.62 (s, 1H), 8.47 (d, J=8.5 Hz, 1H), 8.38 (d, J=7.8 Hz, 1H), 8.31-8.14 (m, 2H), 8.08 (d, J=8.4 Hz, 1H), 8.04 (s, 1H), 7.87 (d, J=7.3 Hz, 1H), 7.73 (s, 1H), 7.56 (d, J=7.8 Hz, 1H), 7.39 (d, J=8.1 Hz, 1H), 7.35 (s, 1H), 7.30-7.20 (m, 2H), 7.06-7.01 (m, 2H), 5.23-5.14 (m, 1H), 4.92 (s, 1H), 4.71-4.67 (m, 1H), 4.60-4.55 (m, 1H), 4.55-4.50 (m, 1H), 4.32-4.28 (m, 1H), 4.19-4.16 (m, 2H), 3.84-3.81 (m, 3H), 3.59-3.56 (m, 2H), 3.43-3.33 (m, 2H), 2.67 (s, 3H), 2.48 (s, 3H), 2.15-2.02 (m, 3H), 1.89-1.77 (m, 3H), 1.56-1.45 (m, 2H), 1.40-1.27 (m, 7H), 1.24-1.18 (m, 2H), 0.95 (s, 9H).

### Example A353. 2-amino-N-((S)-6-(6-(((1S,4S)-4-(2-(2-(((28,4R)-1-((S)-2-(1-cyanocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-3-ethynyl-N-methylquinoline-6-carboxamide

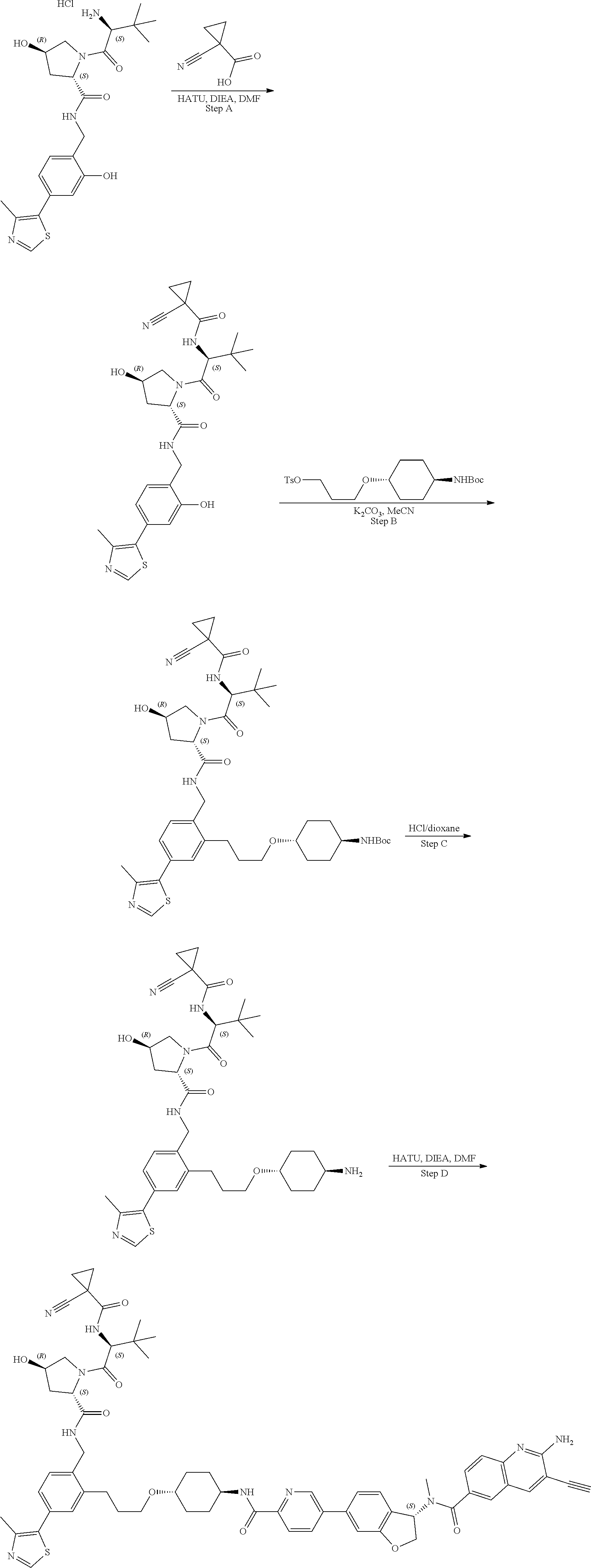

Step A: (2S, 4R)-1-((S)-2-(1-cyanocyclopropane-1-carboxamido)-3,3-dimethylbutanoyl)-4-hydroxy-N-(2-hydroxy-4-(4-methylthiazol-5-yl)benzyl)pyrrolidine-2-carboxamide

To a solution of (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (50 mg, 112 μmol, 1.0 eq), 1-cyanocyclopropane-1-carboxylic acid (12.4 mg, 112 μmol, 1.0 eq.) and HATU (64 mg, 168 μmol, 1.5 eq) in DMF (2.0 mL) was added N-ethyl-N-isopropylpropan-2-amine (43 mg, 336 μmol, 3.0 eq). The reaction was stirred at room temperature for 1 hour. The resulting solution was diluted with 30 mL of water and extracted with EtOAc (30 mL×3). The combined organic layers were washed with brine (30 mL), dried over Na₂SO₄ and concentrated under reduced pressure. The reaction was purified by silica gel chromatography (100-200 mesh silica gel, 0-10% MeOH in DCM) to give (2S,4R)-1-((S)-2-(1-cyanocyclopropane-1-carboxamido)-3,3-dimethylbutanoyl)-4-hydroxy-N-(2-hydroxy-4-(4-methylthiazol-5-yl)benzyl)pyrrolidine-2-carboxamide (60 mg, 99% yield) as a yellow solid. LC-MS (ESI): mass calcd. For C27H₃₃N505S, 539.2; m/z found, 540 [M+H]⁺.

Steps B-D: 2-amino-N-((S)-6-(6-(((1S, 4S)-4-(2-(2-(((2S,4R)-1-((S)-2-(1-cyanocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-3-ethynyl-N-methylquinoline-6-carboxamide

Example A353 was synthesized following the same procedures as described in Example A23 (9 mg, 7% yield over 3 steps) as a yellow solid. LC-MS (ESI): mass calcd, for C₆₂H66N1009S, 1126.5; m/z found, 1128 [M+H]⁺. ¹H NMR (400 MHz, DMSO-d₆) δ 8.99 (s, 1H), 8.92 (s, 1H), 8.67 (s, 1H), 8.52-8.47 (m, 2H), 8.28 (dd, J=8.2, 1.9 Hz, 1H), 8.10-8.05 (m, 2H), 7.90 (d, J=8.8 Hz, 1H), 7.75 (s, 1H), 7.56 (d, J=7.8 Hz, 1H), 7.42-7.34 (m, 4H), 7.06 (s, 1H), 6.97 (d, J=7.8 Hz, 1H), 4.95 (s, 1H), 4.69 (d, J=6.3 Hz, 2H), 4.56-4.50 (m, 3H), 4.35 (s, 1H), 4.31-4.25 (m, 2H), 4.18 (s, 2H), 3.81 (d, J=4.3 Hz, 3H), 3.67-3.55 (m, 2H), 3.37 (t, J=10.0 Hz, 1H), 2.67 (s, 3H), 2.47 (s, 3H), 2.10-2.01 (m, 3H), 1.97-1.91 (m, 1H), 1.86 (d, J=10.7 Hz, 2H), 1.66-1.60 (m, 2H), 1.55-1.47 (m, 4H), 1.36-1.26 (m, 2H), 0.94 (s, 9H).

### Example A354. 2-amino-3-ethynyl-N-((S)-6-(6-((((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)oxy) methyl)pyridin-3-yl)-2,3-dihydrobenzofuran-3-yl)-N-methylquinoline-6-carboxamide

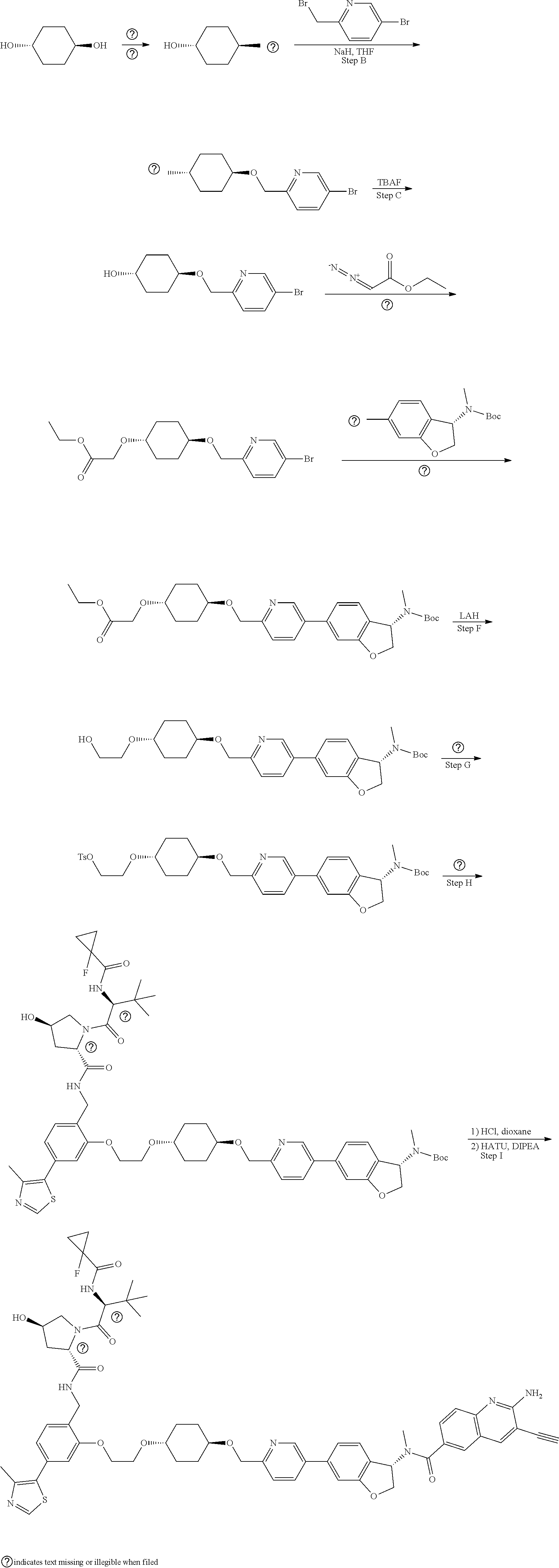

Example A354 was synthesized following the same procedures as described in Example A23 (1.3 mg, 1% yield over 9 steps) as a white solid. LC-MS (ESI): mass calcd, for C₆₁H₆₇FN₈O₉S, 1106.5/z found, 1107.3 [M+H]⁺. ¹H NMR (400 MHz, DMSO-d₆) δ 8.90 (s, 1H), 8.85 (s, 1H), 8.66 (s, 1H), 8.46 (s, 1H), 8.07 (s, 1H), 7.98-7.94 (m, 1H), 7.86 (d, J=8.2 Hz, 1H), 7.82-7.72 (m, 1H), 7.53-7.49 (m, 1H), 7.47 (d, J=7.8 Hz, 1H), 7.38-7.34 (m, 1H), 7.29-7.24 (m, 1H), 7.07 (d, J=1.5 Hz, 1H), 7.04-7.00 (m, 1H), 4.76-4.72 (m, 2H), 4.65-4.60 (m, 1H), 4.51-4.46 (m, 2H), 4.44-4.39 (m, 2H), 4.24-4.20 (m, 2H), 3.92-3.88 (m, 2H), 3.87-3.82 (m, 1H), 3.81-3.77 (m, 1H), 3.62-3.58 (m, 1H), 3.56-3.51 (m, 1H), 2.83-2.74 (m, 2H), 2.49 (s, 3H), 2.25-2.19 (m, 1H), 2.13-2.06 (m, 5H), 2.03 (s, 3H), 1.54-1.41 (m, 4H), 1.37-1.28 (m, 4H), 1.03 (s, 9H).

### Example A355. 2-amino-N-((S)-6-(6-(((1S,4S)-4-(2-((4′-(cyanomethyl)-4-(((28,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamoyl)pyridin-3-yl)-2,3-dihydrobenzofuran-3-yl)-3-ethynyl-N-methylquinoline-6-carboxamide

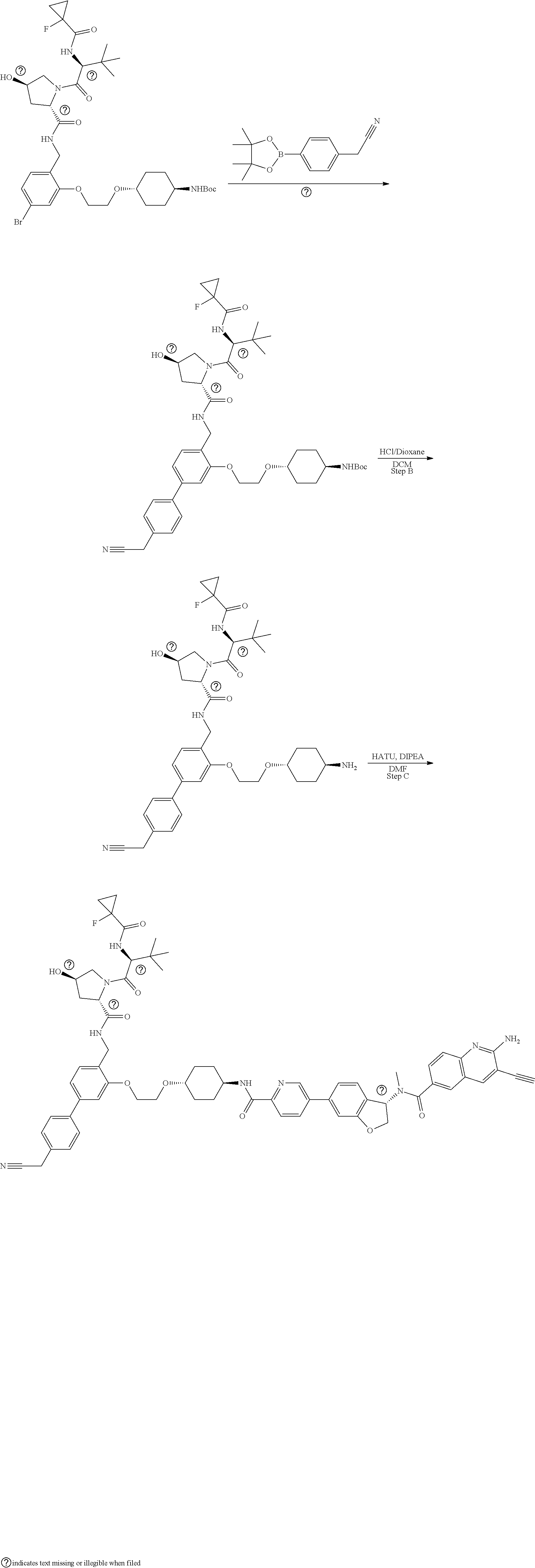

Step A: tert-butyl ((1S, 4r)-4-(2-((4′-(cyanomethyl)-4-(((25, 4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamate

To the solution of tert-butyl ((1S,4r)-4-(2-(5-bromo-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamate (50 mg, 66.2 μmol, 1.0 eq) in 1,4-Dioxane (2 mL) and Water (0.2 mL) were added PdCl₂ (dppf)(4.84 mg, 6.6 μmol, 0.1 eq), 2-(4-(4,4,5,5-tetramethyl-1,3,2-dioxaborolan-2-yl)phenyl) acetonitrile (17.7 mg, 72.8 μmol, 1.1 eq.) and K₂CO₃ (27.4 mg, 198 μmol, 3.0 eq.) and stirred for 1 hour at 80° C. The residue was purified by silica gel cholumn chromatography (100-200 mesh silica gel, 0-10% MeOH in DCM) to afford the tert-butyl ((1S,4r)-4-(2-((4′-(cyanomethyl)-4-(((2S,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamate (40.0 mg, 76% yield) as a colorless oil. LC-MS (ESI): mass calcd. For C43H58FN508, 791; m/z found, 792 [M+H]⁺.

Steps B-C: 2-amino-N-(S)-6-(6-(((1S,4S)-4-(2-((4′-(cyanomethyl)-4-(((2S,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamoyl)pyridin-3-yl)-2,3-dihydrobenzofuran-3-yl)-3-ethynyl-N-methylquinoline-6-carboxamide

Example A355 was synthesized following the same procedures as described in Example A29 (15.7 mg, 28% yield over 2 steps) as a white solid. LC-MS (ESI): mass calcd. for C₆₅H68FN₉O₉, 1137.51; m/z found, 1139.1 [M+H]⁺. ¹H NMR (400 MHz, DMSO-d₆) δ 8.95-8.89 (m, 1H), 8.59 (s, 1H), 8.51-8.41 (m, 2H), 8.30-8.26 (m, 1H), 8.08 (d, J=8.3 Hz, 1H), 8.02 (s, 1H), 7.84 (d, J=8.6 Hz, 1H), 7.71 (d, J=8.2 Hz, 3H), 7.56 (d, J=7.7 Hz, 1H), 7.45-7.34 (m, 5H), 7.30-7.23 (m, 2H), 7.19-7.15 (m, 1H), 5.32 (t, J=4.7 Hz, 1H), 4.88 (s, 1H), 4.71-4.67 (m, 1H), 4.63-4.51 (m, 2H), 4.37-4.34 (m, 1H), 4.31-4.22 (m, 4H), 4.08 (s, 2H), 3.67-3.58 (m, 4H), 3.40-3.35 (m, 1H), 2.67 (s, 3H), 2.13-1.82 (m, 8H), 1.56-1.47 (m, 2H), 1.40-1.29 (m, 4H), 1.24-1.22 (m, 4H), 0.99-0.94 (m, 9H).

### Example A357. 2-amino-3-ethynyl-N-((S)-6-(6-(((1R,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methylquinoline-6-carboxamide

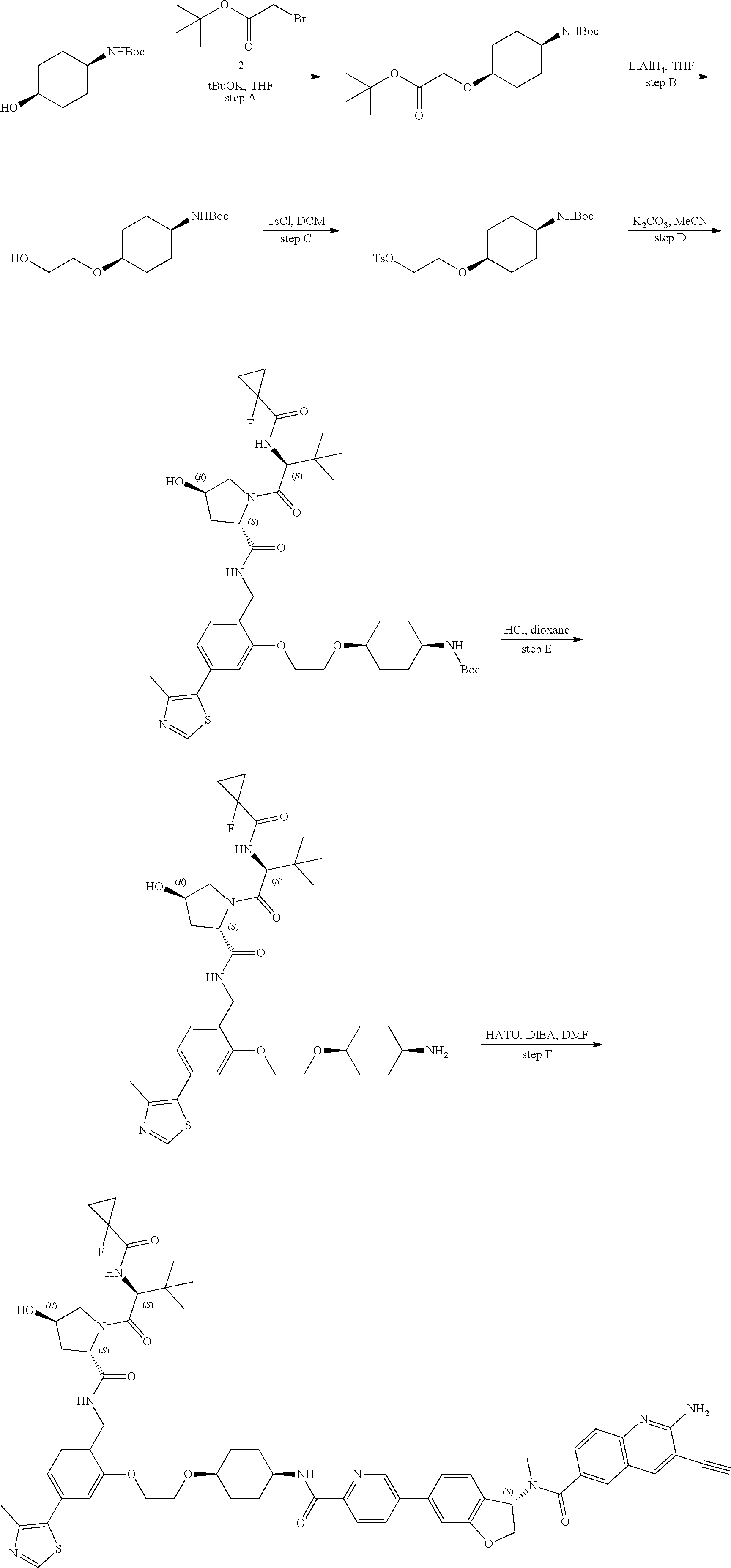

Example A357 was synthesized following the same procedures as described in Example A29 (4.3 mg, 1% yield over 6 steps) as a white solid. LC-MS (ESI): mass calcd, for C₆₁H₆₆FNO₉S, 1119.5/z found, 1121.1 [M+H]⁺. ¹H NMR (400 MHz, DMSO-d₆) δ 8.98 (s, 1H), 8.93 (s, 1H), 8.54-8.45 (m, 1H), 8.35 (d, J=7.9 Hz, 2H), 8.27 (dd, J=8.1, 2.1 Hz, 1H), 8.07 (d, J=8.2 Hz, 1H), 7.90 (s, 1H), 7.71-7.66 (m, 1H), 7.59-7.52 (m, 2H), 7.44-7.37 (m, 2H), 7.34 (s, 1H), 7.27 (d, J=6.6 Hz, 1H), 7.07 (s, 1H), 6.97 (d, J=7.4 Hz, 1H), 4.83-4.64 (m, 3H), 4.60-4.55 (m, 1H), 4.54-4.48 (m, 1H), 4.36-4.29 (m, 2H), 4.27-4.19 (m, 3H), 3.93-3.82 (m, 1H), 3.81-3.75 (m, 2H), 3.65-3.55 (m, 3H), 3.44-3.39 (m, 1H), 2.67 (s, 3H), 2.47 (s, 3H), 2.10-2.02 (m, 1H), 1.95-1.89 (m, 1H), 1.87-1.80 (m, 2H), 1.78-1.69 (m, 2H), 1.69-1.55 (m, 3H), 1.40-1.35 (m, 1H), 1.35-1.31 (m, 1H), 1.25-1.17 (m, 3H), 0.93 (s, 9H).

### Example A358. 2-amino-N-((S)-6-(6-(((1S,4S)-4-(2-((2′,4′-dichloro-4-(((2S,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamoyl)pyridin-3-yl)-2,3-dihydrobenzofuran-3-yl)-3-ethynyl-N-methylquinoline-6-carboxamide

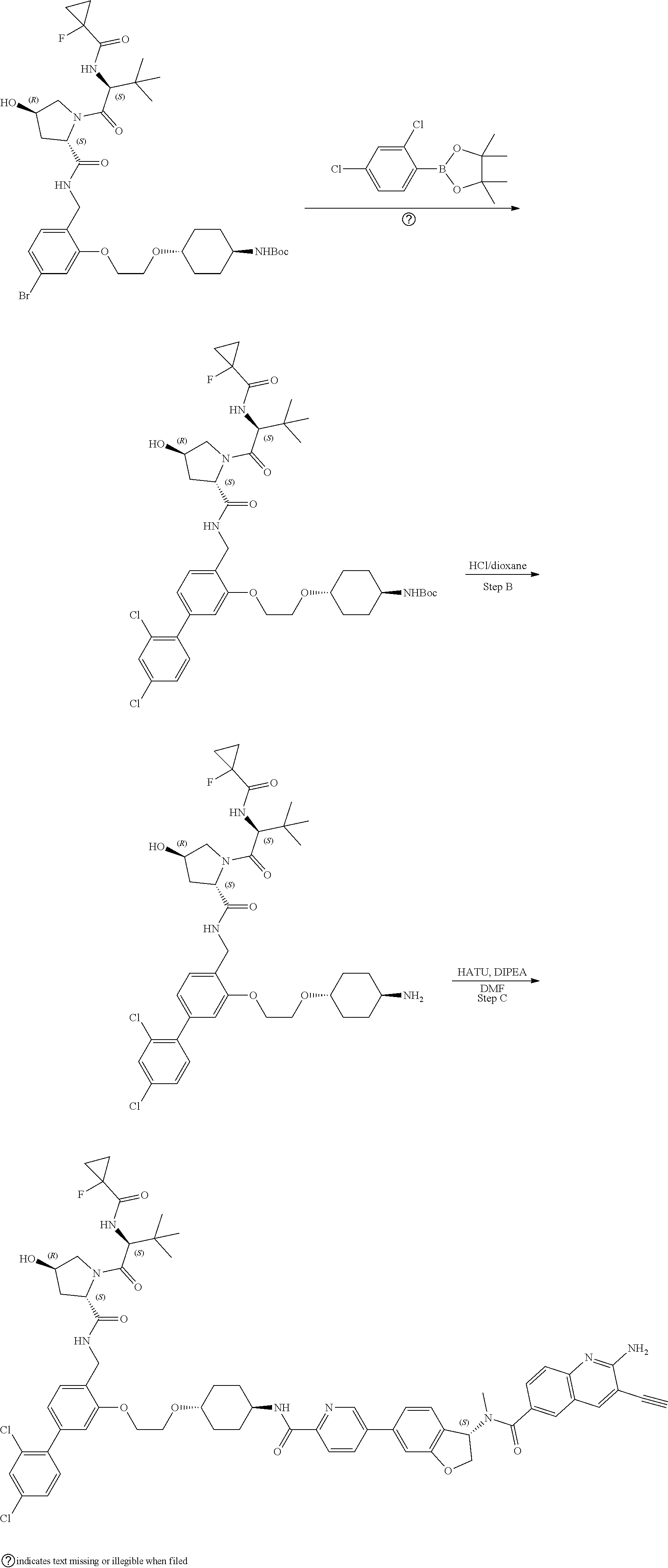

Step A: tert-butyl ((1S,4r)-4-(2-((2′,4′-dichloro-4-(((2S,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamate

To the solution of tert-butyl ((1S,4r)-4-(2-(5-bromo-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamate (50.0 mg, 66.2 μmol, 1.0 eq.) in 1,4-Dioxane (1.00 mL) were added 2-(2,4-dichlorophenyl)-4,4,5,5-tetramethyl-1,3,2-dioxaborolane (19.9 mg, 72.8 μmol, 1.1 eq.), K₂CO₃ (27.4 mg, 198 μmol, 3.0 eq.), PdCl₂ (dppf)(4.84 mg, 6.62 μmol, 0.1 eq.) and water (0.1 mL). The resulting mixture was stirred at 80° C. for 1 hours under the N2 protection. The residue was filtered, concentrated under reduced pressure and purified by cholumn chromatography (silica gel, 0%~10% MeOH in DCM) to afford the tert-butyl ((1S,4r)-4-(2-((2′,4′-dichloro-4-(((2S,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamate (50.0 mg, 92% yield) as a colorless oil. LC-MS (ESI): mass calcd. For C₄₁Hs5C12FN₄Os, 820.34; m/z found, 821.34 [M+H]⁺.

Steps B-C: 2-amino-N-((S)-6-(6-(((1S,4S)-4-(2-((2′,4′-dichloro-4-(((2S,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamoyl)pyridin-3-yl)-2,3-dihydrobenzofuran-3-yl)-3-ethynyl-N-methylquinoline-6-carboxamide

Example A358 was synthesized following the same procedures as described in Example A29 (3.2 mg, 12% yield over 6 steps) as a white solid. LC-MS (ESI): mass calcd. For C₆₃H₆₅C₁₂FN₈O₉, 1166.42; m/z found, 1167.42 [M+H]⁺. ¹H NMR (400 MHz, DMSO-d₆) δ 8.92 (s, 1H), 8.56 (s, 1H), 8.52-8.45 (m, 2H), 8.27 (dd, J=8.2, 2.1 Hz, 1H), 8.08 (d, J=8.2 Hz, 1H), 8.01 (s, 1H), 7.83 (d, J=8.6 Hz, 1H), 7.72 (d, J=2.1 Hz, 1H), 7.68 (d, J=8.2 Hz, 1H), 7.56 (d, J=7.8 Hz, 1H), 7.51 (dd, J=8.3, 2.1 Hz, 1H), 7.45 (d, J=8.3 Hz, 1H), 7.43-7.32 (m, 4H), 7.30 -7.20 (m, 2H), 7.02 (d, J=1.3 Hz, 1H), 6.92 (dd, J=7.8, 1.3 Hz, 1H), 4.86 (s, 1H), 4.71-4.67 (m, 1H), 4.60 (d, J=9.2 Hz, 1H), 4.53 (t, J=8.2 Hz, 1H), 4.36 (s, 1H), 4.31 (d, J=5.9 Hz, 1H), 4.28-4.22 (m, 1H), 4.17-4.13 (m, 2H), 3.83-3.78 (m, 3H), 3.68-3.67 (m, 2H), 3.66-3.64 (m, 2H), 3.39-3.32 (m, 1H), 2.67 (s, 3H), 2.14-2.08 (m, 1H), 2.07-2.02 (m, 2H), 2.00-1.95 (m, 1H), 1.88-1.82 (m, 2H), 1.54-1.47 (m, 2H), 1.39-1.33 (m, 2H), 1.30-1.27 (m, 2H), 1.22-1.19 (m, 2H), 0.96 (s, 9H).

### Example A359. 2-amino-N-((S)-6-(6-(((1S,4S)-4-(2-(2-(((2S,4R)-1-((S)-2-(bicyclo[1.1.1]pentane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-3-ethynyl-N-methylquinoline-6-carboxamide

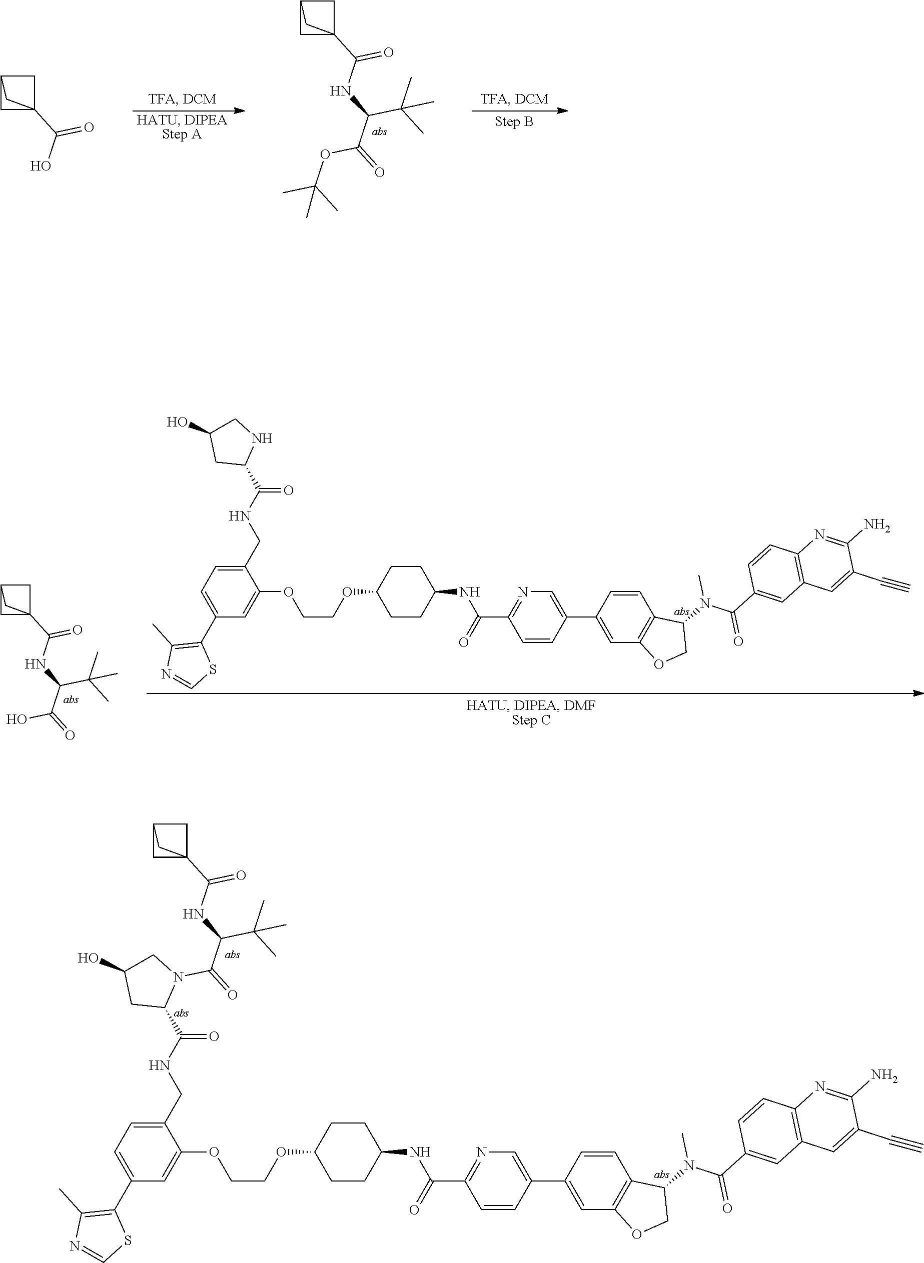

Step A: tert-butyl(S)-2-(bicyclo[1.1.1]pentane-1-carboxamido)-3,3-dimethylbutanoate

To a mixture of bicyclo[1.1.1]pentane-1-carboxylic acid (100 mg, 892 μmol, 1.0 eq.), tert-butyl(S)-2-amino-3,3-dimethylbutanoate (167 mg, 892 μmol, 1.0 eq.) and HATU (509 mg, 1.34 mmol, 1.5 eq.) in DMF (3 mL) was added DIEA (346 mg, 466 μL, 2.68 mmol, 3.0 eq.). The mixture was stirred at room temperature for 2 hours. The mixture was quenched H₂O (30 mL), extracted with EtOAc (30 mL×2) and the layers were separated. The organic layer was washed with brine (30 mL), dried over Na₂SO₄ and concentrated under reduced pressure, the residue was purified by silica gel chromatography (silica gel, 0-30% of EtOAc in PE) to give tert-butyl(S)-2-(bicyclo[1.1.1]pentane-1-carboxamido)-3,3-dimethylbutanoate (110 mg, 43% yield) as a white solid. LC-MS (ESI): mass calcd. For C₁₆H27NO_{3, 281.20}; m/z found, 282.3 [M+H]⁺.

Step B: (S)-2-(bicyclo[1.1.1]pentane-1-carboxamido)-3,3-dimethylbutanoic acid

To a solution of tert-butyl(S)-2-(bicyclo[1.1.1]pentane-1-carboxamido)-3,3-dimethylbutanoate (20 mg, 71.1 μmol, 1.0 eq.) in DCM (4 mL) was added TFA (1 mL). The reaction was stirred at room temperature for 16 hours. The mixture was concentrated to afford(S)-2-(bicyclo[1.1.1]pentane-1-carboxamido)-3,3-dimethylbutanoic acid (15 mg, 93% yield) as a white solid. LC-MS (ESI): mass calcd. For C₁₂H₁₉NO₃, 225.14; m/z found, 226.2 [M+H]⁺.

Step C: 2-amino-N-((S)-6-(6-(((1S,4S)-4-(2-(2-(((2S,4R)-1-((S)-2-(bicyclo[1.1.1]pentane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-3-ethynyl-N-methylquinoline-6-carboxamide

Example A359 was synthesized following the same procedures as described in Example A23 (2.0 mg, 4% yield) as a white solid. The synthetic procedure of 2-amino-3-ethynyl-N-((S)-6-(6-(((1S,4S)-4-(2-(2-(((2S,4R)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide is described in Steps I′-N′ below. LC-MS (ESI): mass calcd, for C₆₃H69N909S, 1127.49; m/z found, 1128.9 [M+H]⁺. ¹HNMR (400 MHz, DMSO-d₆) δ 8.99 (s, 1H), 8.92 (s, 1H), 8.62 (s, 1H), 8.50 (d, J=8.5 Hz, 1H), 8.27 (d, J=8.2 Hz, 1H), 8.14-8.02 (m, 3H), 7.87 (d, J=8.9 Hz, 1H), 7.72 (d, J=7.4 Hz, 1H), 7.56 (d, J=7.6 Hz, 1H), 7.44-7.23 (m, 4H), 7.06-6.95 (m, 2H), 4.91 (s, 1H), 4.82-4.64 (m, 2H), 4.45-4.29 (m, 4H), 4.21-4.13 (m, 3H), 3.85-3.67 (m, 7H), 2.67 (s, 3H), 2.47 (s, 3H), 2.31 (s, 1H), 2.08-2.04 (m, 2H), 2.01-1.98 (m, 2H), 1.89 (s, 6H), 1.58-1.45 (m, 2H), 1.37-1.27 (m, 2H), 1.24 (s, 2H), 1.00-0.87 (m 9H)

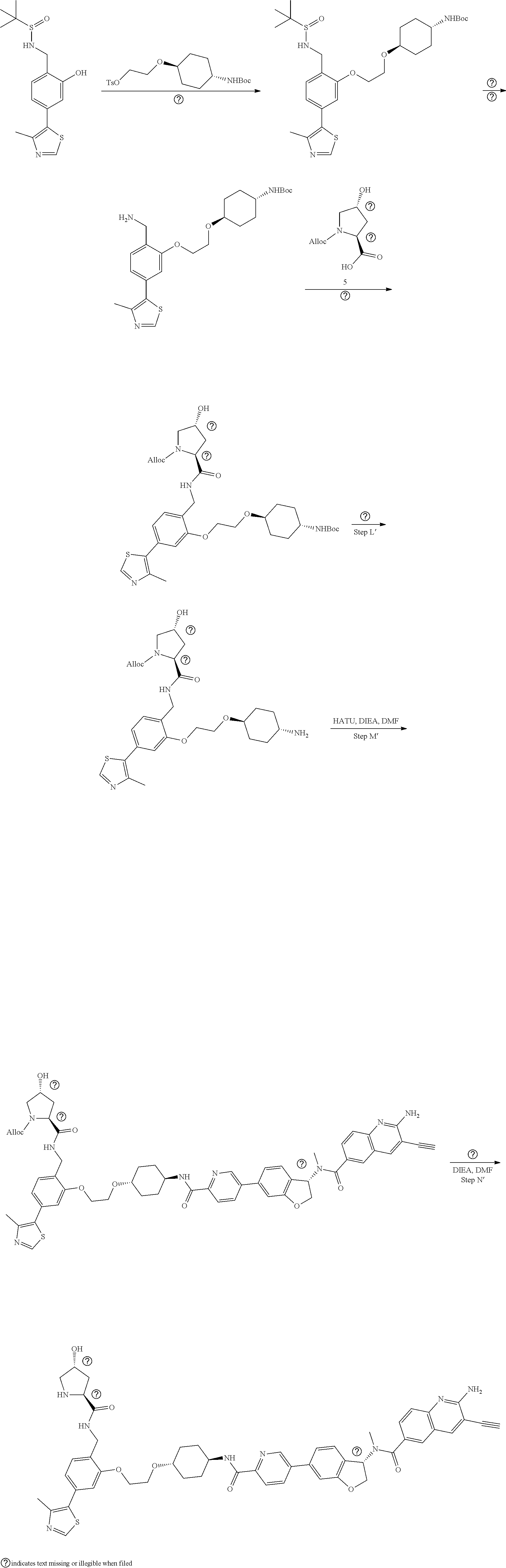

Step I′-J′: tert-butyl ((1r,4r)-4-(2-(2-(aminomethyl)-5-(4-methylthiazol-5-yl)phenoxy)ethoxy)cyclohexyl)carbamate
Tert-butyl ((1r,4r)-4-(2-(2-(aminomethyl)-5-(4-methylthiazol-5-yl)phenoxy)ethoxy)cyclohexyl)carbamate was synthesized following the same procedures as described in Example A126.

Step K′: allyl(2S,4R)-2-((2-(2-(((1r,4S)-4-((tert-butoxycarbonyl)amino)cyclohexyl)oxy)ethoxy)-4-(4-methylthiazol-5-yl)benzyl)carbamoyl)-4-hydroxypyrrolidine-1-carboxylate

To a stirred mixture of tert-butyl ((1r,4r)-4-(2-(2-(aminomethyl)-5-(4-methylthiazol-5-yl)phenoxy)ethoxy)cyclohexyl)carbamate (700 mg, 1.51 mmol, 1.0 eq.) in DMF (20 mL) were added EDCI (581 mg, 3.03 mmol, 2.0 eq.), HOBt (464 mg, 3.03 mmol, 2.0 eq.) and (2S,4R)-1-((allyloxy)carbonyl)-4-hydroxypyrrolidine-2-carboxylic acid (326 mg, 1.52 mmol, 1.0 eq.). Then 4-methylmorfolin (767 mg, 0.83 mL, 7.58 mmol, 5.0 eq.) was added. The mixture was stirred at room temperature for 2 hours. The resulting solution was diluted with 60 mL of water and extracted with EtOAc (60 mL×3). The combined organic layers were washed with brine (50 mL), dried over Na₂SO₄ and concentrated under reduced pressure. The crude product was purified by flash silica chromatography, eluting with a gradient of 0~10% MeOH in DCM to afford allyl(2S,4R)-2-((2-(2-(((1r,4S)-4-((tert-butoxycarbonyl)amino)cyclohexyl)oxy)ethoxy)-4-(4-methylthiazol-5-yl)benzyl)carbamoyl)-4-hydroxypyrrolidine-1-carboxylate (500 mg, 50% yield) as a yellow solid.

LC-MS (ESI): mass calcd, for C₃₃H₄₆N₄O₈S, 658.30; m/z found, 659.4 [M+H]⁺.

Steps L′-M′: allyl(2S,4R)-2-((2-(2-(((1S,4S)-4-(5-((S)-3-(2-amino-3-ethynyl-N-methylquinoline-6-carboxamido)-2,3-dihydrobenzofuran-6-yl)picolinamido)cyclohexyloxy)ethoxy)-4-(4-methylthiazol-5-yl)benzyl)carbamoyl)-4-hydroxypyrrolidine-1-carboxylate

Allyl(2S,4R)-2-((2-(2-(((1S,4S)-4-(5-((S)-3-(2-amino-3-ethynyl-N-methylquinoline-6-carboxamido)-2,3-dihydrobenzofuran-6-yl)picolinamido)cyclohexyl)oxy)ethoxy)-4-(4-methylthiazol-5-yl)benzyl)carbamoyl)-4-hydroxypyrrolidine-1-carboxylate was synthesized following the same procedures as described in Example A136.

Step N′: 2-amino-3-ethynyl-N-((S)-6-(6-(((15,4S)-4-(2-(2-(((2S,4R)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

To a mixture of allyl(2S,4R)-2-((2-(2-(((1S,4S)-4-(5-((S)-3-(2-amino-3-ethynyl-N-methylquinoline-6-carboxamido)-2,3-dihydrobenzofuran-6-yl)picolinamido)cyclohexyl)oxy) ethoxy)-4-(4-methylthiazol-5-yl)benzyl)carbamoyl)-4-hydroxypyrrolidine-1-carboxylate (150 mg, 149.2 μmol, 1.0 eq.), Bis-(triphenylphosphino)-palladous chloride (21 mg, 29.85 μmol, 0.2 eq.) and Meldrum's acid (65 mg, 447.7 μmol, 3.0 eq.) in DMF (5 mL) was added DIEA (58 mg, 78.0 μL, 447.7 μmol, 3.0 eq.) and Triethylsilane (52 mg, 71.5 μL, 447.7 μmol, 3.0 eq.). The mixture was stirred at room temperature for 1 hour. The mixture was quenched water (20 mL), extracted with EtOAc (10 mL×3) and the layers were separated. The organic layer was washed with brine, dried over Na₂SO₄ and concentrated under reduced pressure, the residue was purified by silica gel chromatography (100-200 mesh silica gel, 0-20% of MeOH in DCM) to give 2-amino-3-ethynyl-N-((S)-6-(6-(((1S,4S)-4-(2-(2-(((2S,4R)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide (20 mg, 15% yield) as a yellow solid. LC-MS (ESI): mass calcd, for C₅₁H₅₂N₈O₇S, 920.37; m/z found, 921.5 [M+H]⁺.

### Example A360. N-((3S)-6-(6-(((18,4S)-4-(2-(2-(((2S,4R)-1-((2S)-2-((1R,3R,5S)-adamantane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-2-amino-3-ethynyl-N-methylquinoline-6-carboxamide

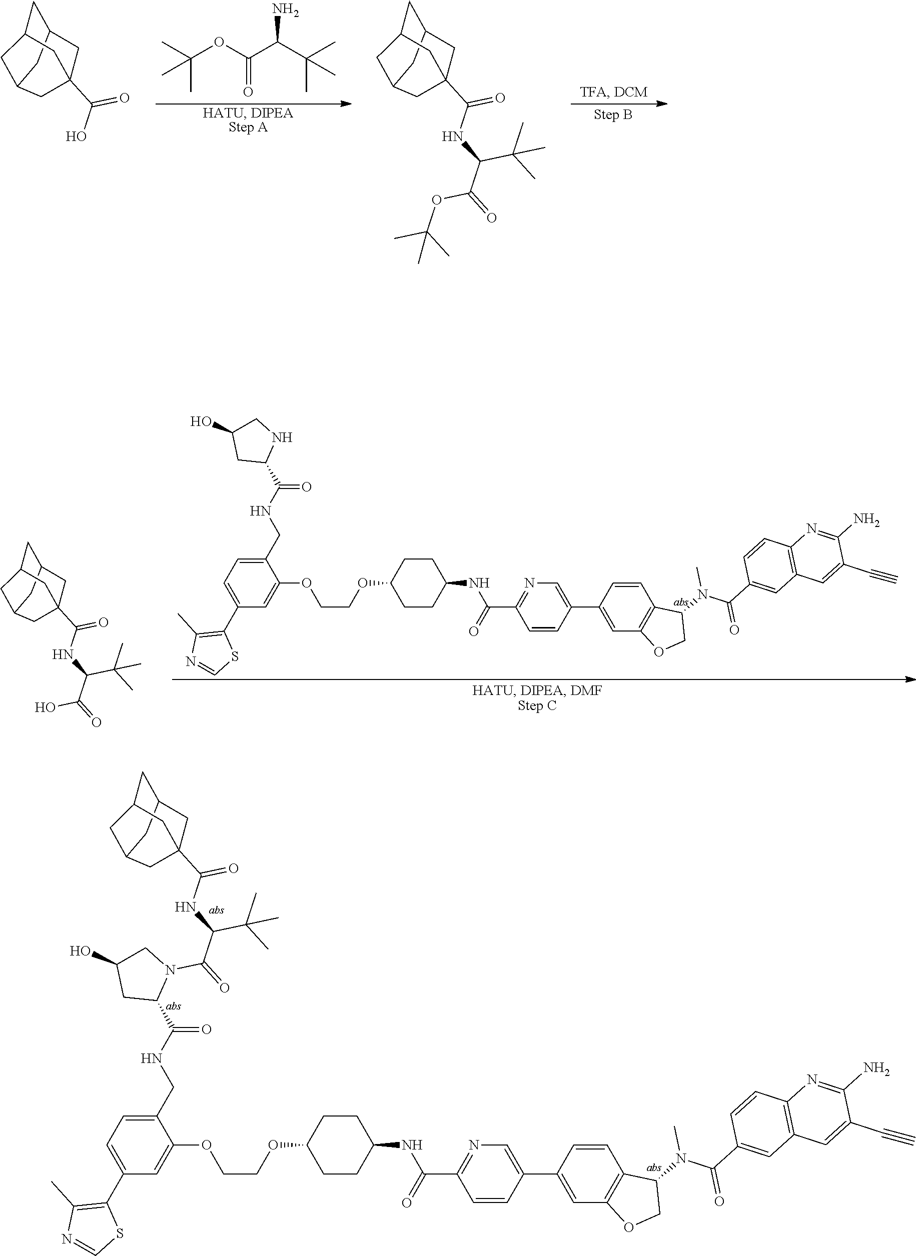

Example A360 was synthesized following the same procedures as described in Example A373 (2.0 mg, 2% yield over 3 steps) as a white solid. LC-MS (ESI): mass calcd, for C₆₈H₇₇N₉O₉S, 1195.56; m/z found, 1197.1 [M+H]⁺. ¹H NMR (400 MHz, DMSO-d₆) δ 8.99 (d, J=3.7 Hz, 1H), 8.92 (d, J=1.7 Hz, 1H), 8.66 (s, 1H), 8.53-8.41 (m, 2H), 8.27 (dd, J=8.3, 2.0 Hz, 1H), 8.16-7.95 (m, 3H), 7.89 (dd, J=8.5, 1.5 Hz, 1H), 7.75 (s, 1H), 7.56 (d, J=7.7 Hz, 1H), 7.42-7.27 (m, 3H), 7.06-7.01 (m, 1H), 6.99-6.94 (m, 1H), 6.79-6.65 (m, 1H), 4.94 (s, 1H), 4.84-4.66 (m, 2H), 4.57-4.25 (m, 5H), 4.21-4.13 (m, 3H), 3.84-3.75 (m, 6H), 2.96 (s, 1H), 2.66 (s, 3H), 2.46 (s, 3H), 2.06-2.02 (m, 2H), 1.98-1.94 (m, 2H), 1.91-1.89 (m, 1H), 1.83-1.78 (m, 4H), 1.67-1.64 (m, 6H), 1.56-1.48 (m, 6H), 1.38-1.22 (m, 3H), 1.01-0.88 (m, 9H).

### Example A363. 2-amino-3-ethynyl-N-((S)-6-(6-(((1R,4S)-4-(2-(2-((R)-1-((2S,4R)-1-((S)-2-(1-fluorocyclopropane-1-carboxamido)-3,3-dimethylbutanoyl)-4-hydroxypyrrolidine-2-carboxamido)-2-hydroxyethyl)-5-(4-methylthiazol-5-yl)phenoxy)ethoxy)cyclohexyl) carbamoyl)pyridin-3-yl)-2,3-dihydrobenzofuran-3-yl)-N-methylquinoline-6-carboxamide

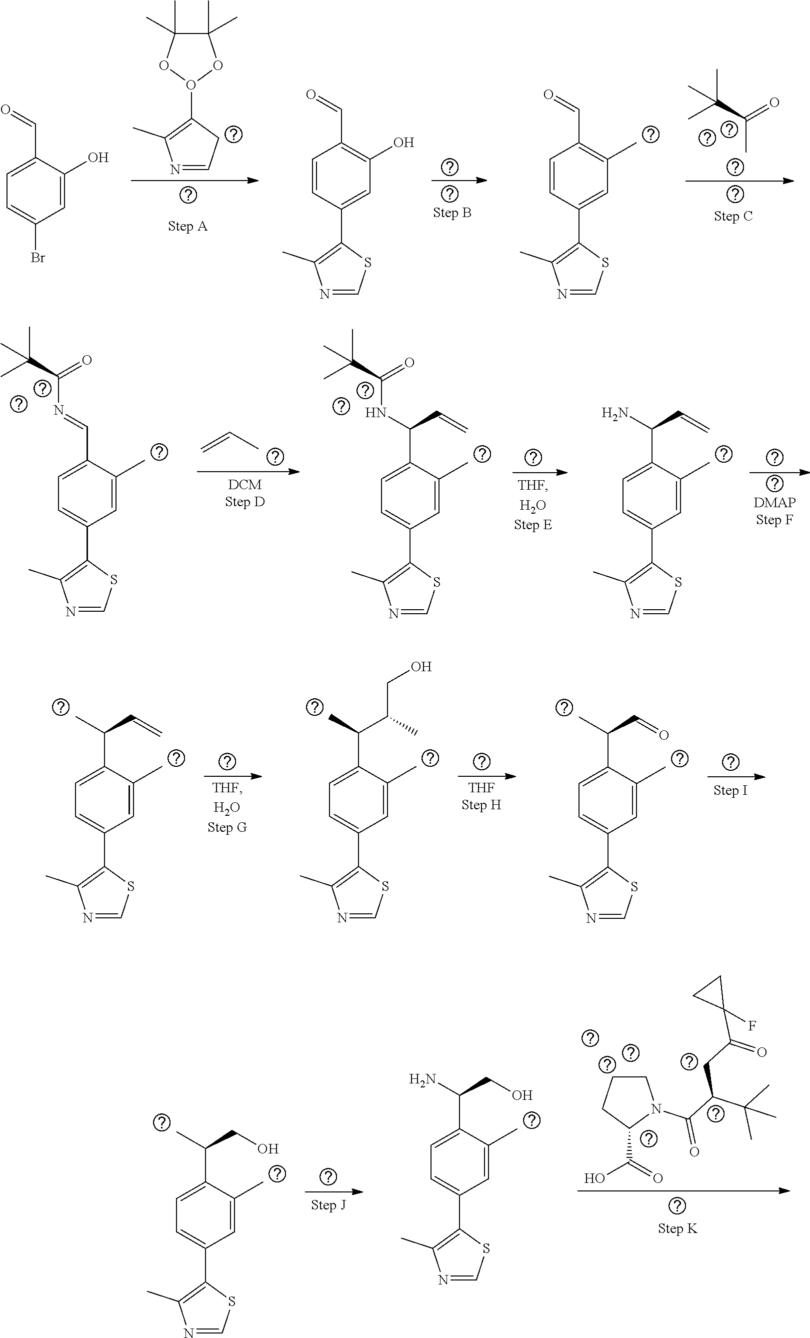

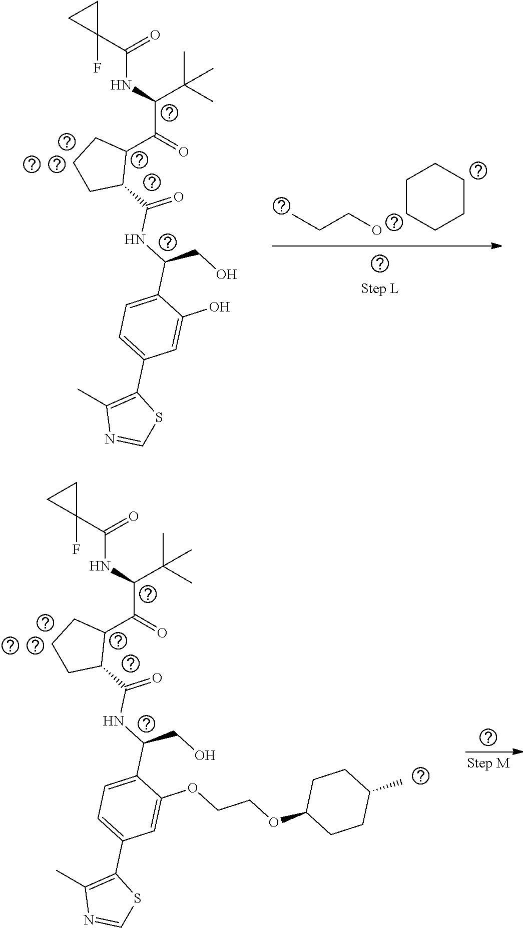

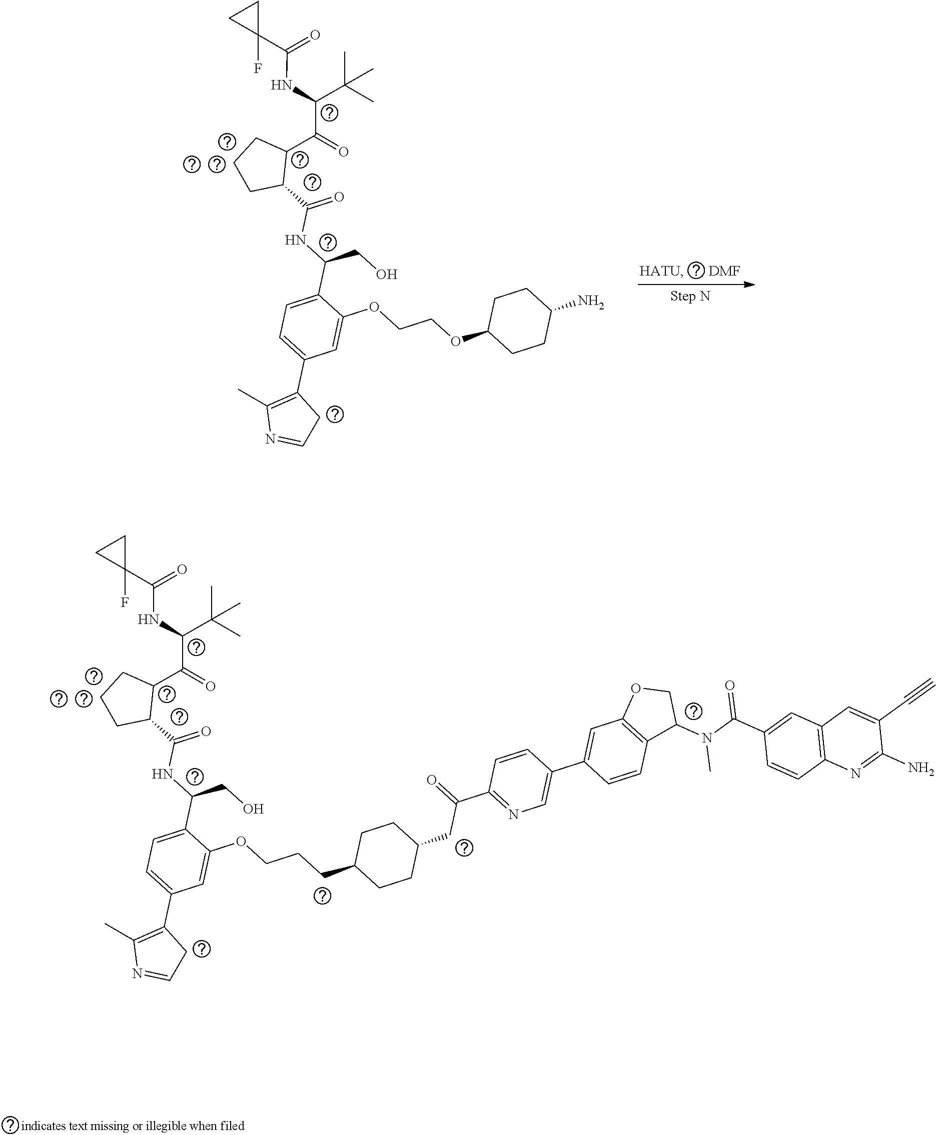

Step A: 2-hydroxy-4-(4-methylthiazol-5-yl)benzaldehyde

To a mixture of 4-bromo-2-hydroxybenzaldehyde (2.0 g, 9.95 mmol, 1.0 eq.) in 1,4-Dioxane (20 mL) and Water (1 mL) was added K₂CO₃ (4.1 g, 29.8 mmol, 3.0 eq.), 4-methyl-5-(4,4,5,5-tetramethyl-1,3,2-dioxaborolan-2-yl) thiazole (2.5 g, 10.9 mmol, 1.1 eq.) and PdCl₂ (dppf) (728 mg, 995 μmol, 0.1 eq.) under N₂. The mixture was stirred at 100° C. for 8 hours. The mixture was quenched water (50 mL), extracted with EtOAc (30 mL×3) and the layers were separated. The organic layer was washed with brine (50 mL), dried over Na₂SO₄, filtered and concentrated. The product was purified by column chromatography (silica gel, 0% EtOAc in PE~20% EtOAc in PE) to afford 2-hydroxy-4-(4-methylthiazol-5-yl)benzaldehyde (2.0 g, 92% yield) as a yellow solid. LC-MS (ESI): mass calcd. For C₁₁H₉NO₂S, 219.03; m/z found, 217.5 [M−H]

Step B: 2-(methoxymethoxy)-4-(4-methylthiazol-5-yl)benzaldehyde

To a mixture of 2-hydroxy-4-(4-methylthiazol-5-yl)benzaldehyde (2.2 g, 10.0 mmol, 1.0 eq.) in DCM (44 mL) was added triethylamine (3.0 g, 30.1 mmol, 3.0 eq.) and MOM-CI (1.6 g, 1.52 mL, 20.1 mmol, 2.0 eq.). The reaction mixture was stirred at room temperature for 3 hours. The mixture was quenched water (50 mL), extracted with EtOAc (30 mL×3) and the layers were separated. The organic layer was washed with brine (50 mL), dried over Na₂SO₄, filtered and concentrated. The product was purified by column chromatography (silica gel, 0% EtOAc in PE~30% EtOAc in PE) to afford 2-(methoxymethoxy)-4-(4-methylthiazol-5-yl)benzaldehyde (1.40 g, 53% yield) as a yellow oil. LC-MS (ESI): mass calcd. For C13H13NO₃S, 263.06; m/z found, 263.6 [M+H]⁺.

Step C: (R,E)-N-(2-(methoxymethoxy)-4-(4-methylthiazol-5-yl)benzylidene)-2-methylpropane-2-sulfinamide

To a mixture of 2-(methoxymethoxy)-4-(4-methylthiazol-5-yl)benzaldehyde (900 mg, 3.42 mmol, 1.0 eq.) in THF (20 mL) was added (R)-2-methylpropane-2-sulfinamide (829 mg, 6.84 mmol, 2.0 eq.) and cesium carbonate (3.3 g, 10.3 mmol, 3.0 eq.). The reaction mixture was stirred at 50° C. for 16 hours. The mixture was concentrated and purified by column chromatography (silica gel, 0% EtOAc in PE~45% EtOAc in PE) to afford (E)-N-(2-(methoxymethoxy)-4-(4-methylthiazol-5-yl)benzylidene)-2-methylpropane-2-sulfinamide (1.1 g, 90% yield) as a yellow solid. LC-MS (ESI): mass calcd. For C₁₇H22N203S2, 366.11; m/z found, 367.1 [M+H]⁺.

Step D: (R)-N-((S)-1-(2-(methoxymethoxy)-4-(4-methylthiazol-5-yl)phenyl)allyl)-2-methylpropane-2-sulfinamide

To a mixture of (E)-N-(2-(methoxymethoxy)-4-(4-methylthiazol-5-yl)benzylidene)-2-methylpropane-2-sulfinamide (1.5 g, 4.09 mmol, 1.0 eq.) in DCM (15 mL) was added vinylmagnesium bromide (1.1 g, 8.20 mL, 8.19 mmol, 2.0 eq.). The reaction mixture was stirred at −78° C. for 2 hours. The mixture was quenched aq. NH₄Cl (50 mL), extracted with DCM (30 mL×3) and the layers were separated. The organic layer was washed with brine, dried over Na₂SO₄, filtered and concentrated. The product was purified by prep-HPLC (C18, MeCN/H2O, 0.1% FA, 0 to 55% gradient, 30 min run) to afford N-((S)-1-(2-(methoxymethoxy)-4-(4-methylthiazol-5-yl)phenyl)allyl)-2-methylpropane-2-sulfinamide (900 mg, 56% yield) as a yellow oil. LC-MS (ESI): mass calcd. For C₁₉H26N₂O₃S2, 394.14; m/z found, 395.0 [M+H]⁺.

Step E: (S)-1-(2-(methoxymethoxy)-4-(4-methylthiazol-5-yl)phenyl) prop-2-en-1-amine

To a mixture of N-((S)-1-(2-(methoxymethoxy)-4-(4-methylthiazol-5-yl)phenyl)allyl)-2-methylpropane-2-sulfinamide (800 mg, 2.03 mmol, 1.0 eq.) in THF (20 mL) and Water (5 mL) was added I₂ (1.0 g, 4.06 mmol, 2.0 eq.). The reaction mixture was stirred at 50° C. for 1 hour. The reaction solution is directly used for the next step. LC-MS (ESI): mass calcd. For C₁₅H₁₅N₂O₂S, 290.11; m/z found, 290.7 [M+H]⁺.

Step F: tert-butyl(S)-(1-(2-(methoxymethoxy)-4-(4-methylthiazol-5-yl)phenyl)allyl)carbamate

To a mixture of(S)-1-(2-(methoxymethoxy)-4-(4-methylthiazol-5-yl)phenyl) prop-2-en-1-amine (589 mg, 2.03 mmol, 1.0 eq.) in THF (20 mL) and Water (5 mL) was added Sodium bicarbonate (1.0 g, 473 μL, 12.2 mmol, 6.0 eq.) and Di-tert-butyl dicarbonate (531 mg, 544 μL, 2.43 mmol, 1.2 eq.). The reaction mixture was stirred at room temperature for 1 hour. The mixture was quenched water (50 mL), extracted with EtOAc (30 mL×3) and the layers were separated. The organic layer was washed with brine (50 mL), dried over Na₂SO₄, filtered and concentrated.

The product was purified by column chromatography (silica gel, 0% EtOAc in PE~50% EtOAc in PE) to afford tert-butyl(S)-(1-(2-(methoxymethoxy)-4-(4-methylthiazol-5-yl)phenyl)allyl)carbamate (570 mg, 72% yield) as a yellow oil. LC-MS (ESI): mass calcd. For C₂₀H₂₆N₂O4S, 390.17; m/z found, 390.8 [M+H]⁺

Step G: tert-butyl ((1R,2R)-2,3-dihydroxy-1-(2-(methoxymethoxy)-4-(4-methylthiazol-5-yl)phenyl) propyl)carbamate

To a mixture of tert-butyl(S)-(1-(2-(methoxymethoxy)-4-(4-methylthiazol-5-yl)phenyl)allyl)carbamate (200 mg, 512 μmol, 1.0 eq.) in t-BuOH (6 mL) and H₂O (3 mL) was added Potassiumosmate (VI) dihydrate (19 mg, 51.2 μmol, 0.1 eq.) and NMO (126 mg, 1.08 mmol, 2.1 eq.). The reaction mixture was stirred at room temperature for 2 hours. The mixture was quenched aq. Na₂S₂O₃ (30 mL.), extracted with DCM (30 mL×3) and the layers were separated. The organic layer was washed with brine (30 mL), dried over Na₂SO₄, filtered and concentrated. The product was purified by column chromatography (silica gel, 0% EtOAc in PE~85% EtOAc in PE) to afford tert-butyl ((1R,2R)-2,3-dihydroxy-1-(2-(methoxymethoxy)-4-(4-methylthiazol-5-yl)phenyl) propyl)carbamate (180 mg, 83% yield) as a yellow oil. LC-MS (ESI): mass calcd. For C₂₀H₂₈N₂O₆S, 424.17; m/z found, 424.7 [M+H]⁺.

Step H: tert-butyl (R)-(1-(2-(methoxymethoxy)-4-(4-methylthiazol-5-yl)phenyl)-2-oxoethyl)carbamate

To a mixture of tert-butyl ((1R,2R)-2,3-dihydroxy-1-(2-(methoxymethoxy)-4-(4-methylthiazol-5-yl)phenyl) propyl)carbamate (180 mg, 424 μmol, 1.0 eq.) in THF (2 mL) and Water (1 mL) was added tert-butyl ((1R,2R)-2,3-dihydroxy-1-(2-(methoxymethoxy)-4-(4-methylthiazol-5-yl)phenyl) propyl)carbamate (180 mg, 424 μmol, 1.0 eq.). The reaction mixture was stirred at room temperature for 2 hours. The mixture was quenched Na₂S₂O₃ (30 mL), extracted with DCM (30 mL×3) and the layers were separated. The organic layer was washed with brine (30 mL), dried over Na₂SO₄, filtered and concentrated to afford tert-butyl (R)-(1-(2-(methoxymethoxy)-4-(4-methylthiazol-5-yl)phenyl)-2-oxoethyl)carbamate (166 mg, 99% yield) as a yellow oil. LC-MS (ESI): mass calcd. For C₁₉H₂₄N205S, 392.14; m/z found, 392.7 [M+H]⁺.

Step I: tert-butyl (R)-(2-hydroxy-1-(2-(methoxymethoxy)-4-(4-methylthiazol-5-yl)phenyl)ethyl)carbamate

To a mixture of tert-butyl (R)-(1-(2-(methoxymethoxy)-4-(4-methylthiazol-5-yl)phenyl)-2-oxoethyl)carbamate (165 mg, 420.4 μmol, 1.0 eq.) in MeOH (5 mL) was added sodium tetrahydroborate (64 mg, 1.682 mmol, 4.0 eq.) at 0° C. The reaction mixture was stirred at room temperature for 1 hour. The mixture was quenched water (30 mL), extracted with DCM (30 mL×3) and the layers were separated. The organic layer was washed with brine (30 mL), dried over Na₂SO₄, filtered and concentrated. The product was purified by column chromatography (silica gel, 0% EtOAc in PE~70% EtOAc in PE) to afford tert-butyl (R)-(2-hydroxy-1-(2-(methoxymethoxy)-4-(4-methylthiazol-5-yl)phenyl)ethyl)carbamate (130 mg, 78% yield) as a yellow oil. LC-MS (ESI): mass calcd. For C₁₉H₂₆N₂O₅S, 394.16; m/z found, 394.7 [M+H]⁺.

Step J: (R)-2-(1-amino-2-hydroxyethyl)-5-(4-methylthiazol-5-yl) phenol

To a mixture of tert-butyl (R)-(2-hydroxy-1-(2-(methoxymethoxy)-4-(4-methylthiazol-5-yl)phenyl)ethyl)carbamate (130 mg, 329.5 μmol, 1.0 eq.) in DCM (2 mL) was added HCl/1,4-dioxane (2.0 mL, 4 M/L). The reaction mixture was stirred at room temperature for 1 hour. The mixture was concentrated to afford (R)-2-(1-amino-2-hydroxyethyl)-5-(4-methylthiazol-5-yl) phenol (82 mg, 99% yield) as a white solid. LC-MS (ESI): mass calcd. For C₁₂H14N202S, 250.08; m/z found, 250.7 [M+H]⁺.

Steps K-N: 2-amino-3-ethynyl-N-(S)-6-16-(((IR, 4S)-4-(2-(2-((R)-1-(28,4R)-1-(S)-2-(1-fluorocyclopropane-1-carboxamido)-3,3-dimethylbutanoyl)-4-hydroxypyrrolidine-2-carboxamido)-2-hydroxyethyl)-5-(4-methylthiazol-5-yl)phenoxy)ethoxy)cyclohexyl) carbamoyl)pyridin-3-yl)-2,3-dihydrobenzofuran-3-yl)-N-methylquinoline-6-carboxamide

Example A363 was synthesized following the same procedures as described in Example A23 (13.0 mg, 3% yield over 4 steps) as a white solid. LC-MS (ESI): mass calcd. for C₆₂H₆₈FN₉O₁₀S, 1149.48; m/z found, 1148.3 [M−H]⁻. ¹H NMR (400 MHz, DMSO-d₆) δ 8.99 (s, 1H), 8.92 (s, 1H), 8.70 (s, 2H), 8.44 (d, J=8.5 Hz, 1H), 8.35-8.24 (m, 2H), 8.11-8.05 (m, 2H), 7.92 (dd, J=8.6, 1.4 Hz, 1H), 7.75 (d, J=7.5 Hz, 1H), 7.56 (d, J=7.8 Hz, 1H), 7.39 (d, J=6.8 Hz, 1H), 7.34 (s, 1H), 7.30 (d, J=7.8 Hz, 1H), 7.25 (dd, J=9.0, 2.4 Hz, 1H), 7.09-6.98 (m, 2H), 5.26-5.18 (m, 1H), 4.97 (s, 1H), 4.88-4.77 (m, 1H), 4.69 (d, J=6.5 Hz, 1H), 4.60-4.55 (m, 2H), 4.30 (s, 1H), 4.17 (d, J=2.6 Hz, 2H), 3.84-3.79 (m, 2H), 3.69-3.65 (m, 2H), 3.60 (d, J=5.8 Hz, 2H), 3.57-3.54 (m, 1H), 3.43-3.31 (m, 2H), 2.66 (s, 3H), 2.48 (s, 3H), 2.15-2.02 (m, 3H), 1.89-1.79 (m, 3H), 1.56-1.45 (m, 2H), 1.39-1.26 (m, 4H), 1.23-1.17 (m, 2H), 0.95 (s, 9H).

### Example A364. 4-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N,3-dimethylimidazo[1,5-a]quinoxaline-8-carboxamide

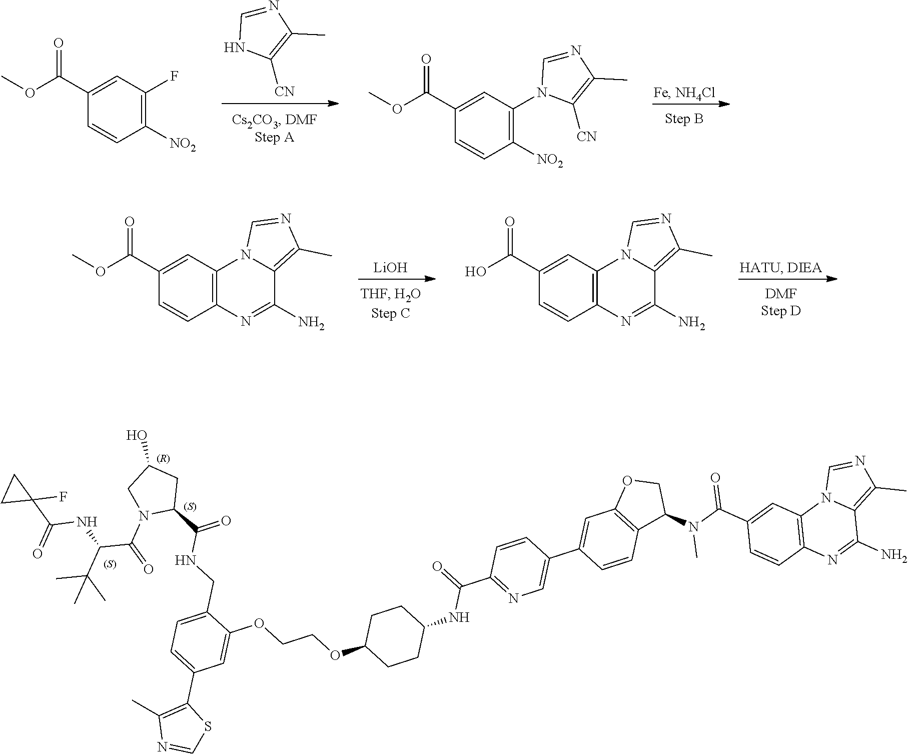

Step A: methyl 3-(5-cyano-4-methyl-1H-imidazol-1-yl)-4-nitrobenzoate

To a solution of methyl 3-fluoro-4-nitrobenzoate (1.5 g, 7.5 mmol, 1.0 eq.) in DMF (30 mL) was added 4-methyl-1H-imidazole-5-carbonitrile (968 mg, 9.0 mmol, 1.2 eq.) and Cs₂CO₃ (2.9 g, 9.0 mmol, 1.2 eq.). The mixture was stirred at 80° C. for 2 hours under N₂ atmosphere. The reaction mixture was poured into water (20 mL) and then extracted with EtOAc (20 mL×2). The combined organic layers were washed with brine, dried over anhydrous Na₂SO₄ and concentrated to afford crude methyl 3-(5-cyano-4-methyl-1H-imidazol-1-yl)-4-nitrobenzoate (650 mg, 30% yield) as a yellow oil. Which used directly for next step without purification. LC-MS (ESI): mass calcd. For C₁₃H₁₀N₄O₄, 286.07; m/z found, 287.1 [M+H]⁺.

Step B: methyl 4-amino-3-methylimidazo[1,5-a]quinoxaline-8-carboxylate

To a solution of crude methyl 3-(5-cyano-4-methyl-1H-imidazol-1-yl)-4-nitrobenzoate (643 mg, 2.2 mmol, 1.0 eq.) in EtOH (9 mL): Water (3 mL) was added NH₄Cl (721 mg, 13.2 mmol, 6.0 eq.) and Fe (753 mg, 13.2 mmol, 6.0 eq.). The mixture was stirred at 80° C. for 3 hours under N₂ atmosphere. The mixture was filtered through a pad of celite and added water (20 mL) and then extracted with EtOAc (20 mL×2). The combined organic layers were washed with brine, dried over anhydrous Na₂SO₄ and concentrated. The residue was purified by column chromatography (100-200 mesh silica gel, 0%~10% MeOH in DCM) to afford methyl 4-amino-3-methylimidazo[1,5-a]quinoxaline-8-carboxylate (35 mg, 6% yield) as a white solid. as a white solid.LC-MS (ESI): mass calcd. For C₁₃H₁₂N₄O₂,256.10; m/z found, 257.2 [M+H]⁺.

Steps C-D: 4-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N,3-dimethylimidazo[1,5-a]quinoxaline-8-carboxamide

Example A364 was synthesized following the same procedures as described in Example A23 (3.8 mg, 4% yield over 2 steps) as a white solid. LC-MS (ESI): mass calcd. for C₆₁H₆₈FN₁₁O₉S, 1149.49; m/z found, 1151.0 [M+H]⁺. ¹H NMR (400 MHz, DMSO-d₆) δ 9.11-9.04 (m, 1H), 8.90 (s, 1H), 8.67-8.54 (m, 2H), 8.44 (d, J=8.4 Hz, 1H), 8.30-8.22 (m, 1H), 8.09-7.99 (m, 2H), 7.92-7.82 (m, 1H), 7.78-7.66 (m, 1H), 7.60-7.51 (m, 1H), 7.44 (d, J=3.2 Hz, 1H), 7.38 (d, J=7.3 Hz, 1H), 7.34 (s, 1H), 7.27 (d, J=6.8 Hz, 1H), 7.22 (d, J=7.5 Hz, 1H), 7.01 (d, J=7.4 Hz, 1H), 6.37 (d, J=3.2 Hz, 1H), 4.94 (s, 1H), 4.88-4.79 (m, 1H), 4.71-4.66 (m, 1H), 4.65-4.57 (m, 2H), 4.54-4.47 (m, 2H), 4.42-4.32 (m, 2H), 3.76-3.73 (m, 2H), 3.68-3.66 (m, 2H), 3.63-3.61 (m, 2H), 3.21-3.00 (m, 2H), 2.66 (s, 3H), 2.38-2.30 (m, 4H), 2.13-2.03 (m, 1H), 1.98-1.83 (m, 3H), 1.81-1.69 (m, 2H), 1.45-1.29 (m, 5H), 1,25-1.13 (m, 4H), 1.00 (s, 9H).

### Example A365. 2-amino-N-((S)-6-(6-(((1S,4S)-4-(2-(2-(((2S,4R)-1-((S)-2-(1-(difluoromethyl)cyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-3-ethynyl-N-methylquinoline-6-carboxamide

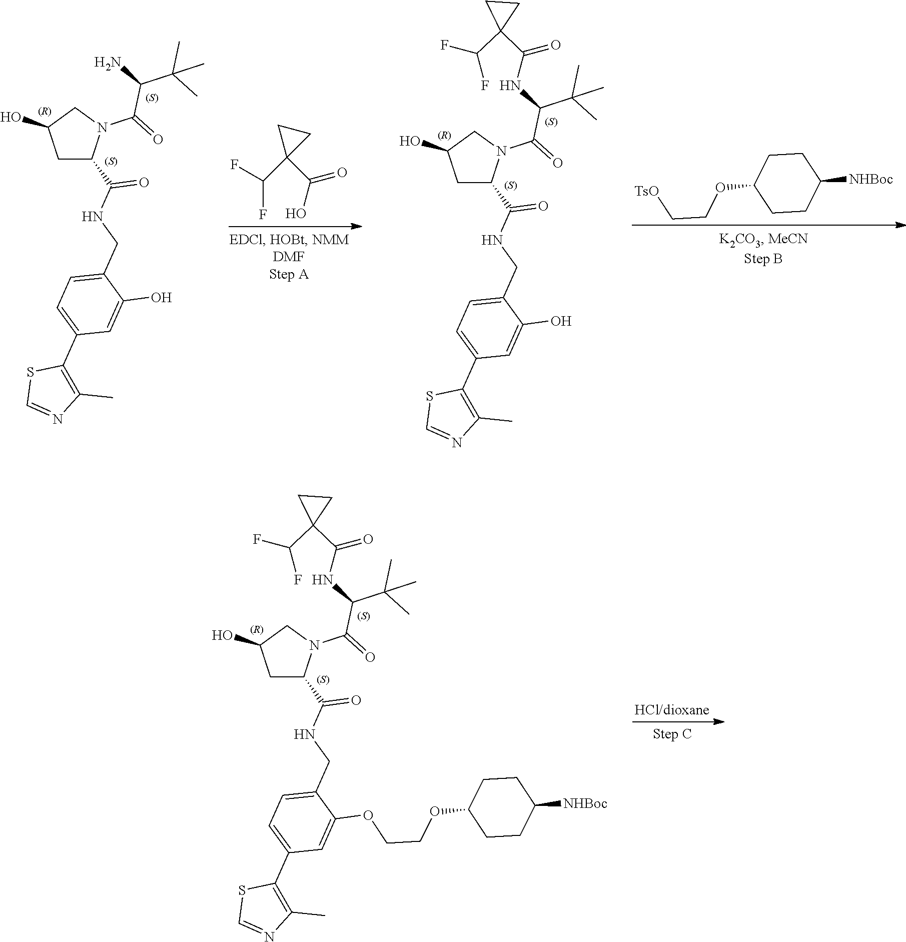

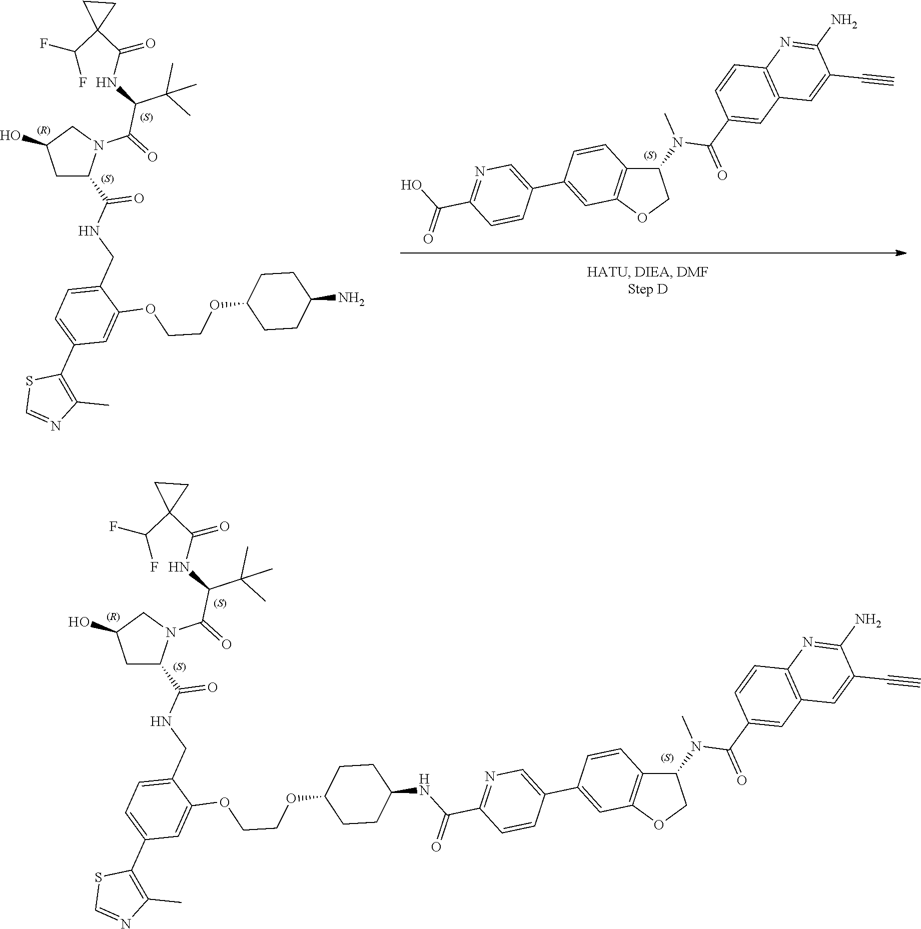

Step A: (2S,4R)-1-((S)-2-(1-(difluoromethyl)cyclopropane-1-carboxamido)-3,3-dimethylbutanoyl)-4-hydroxy-N-(2-hydroxy-4-(4-methylthiazol-5-yl)benzyl)pyrrolidine-2-carboxamide

To a mixture of (2S,4R)-1-((S)-2-amino-3,3-dimethylbutanoyl)-4-hydroxy-N-(2-hydroxy-4-(4-methylthiazol-5-yl)benzyl)pyrrolidine-2-carboxamide (50 mg, 112 μmol, 1.0 eq.), EDCI (35 mg, 224 μmol, 2.0 eq), 1-(difluoromethyl)cyclopropane-1-carboxylic acid (18 mg, 134 μmol, 1.2 eq.) and HOBt (34 mg, 224 μmol, 2.0 eq.) in DMF (3 mL) was added 4-methylmorfolin (23 mg, 224 μmol, 2.0 eq.). The mixture was stirred at room temperature for 2 hours under N₂ atmosphere. The resulting solution was diluted with 10 mL of water and extracted with EtOAc (20 mL×3). The combined organic layers were washed with brine, dried over anhydrous Na₂SO₄ and concentrated under reduced pressure. The residue was purified by flash column chromatography on silica gel (100-200 mesh silica gel, 0-10% MeOH in DCM) to afford (2S,4R)-1-((S)-2-(1-(difluoromethyl)cyclopropane-1-carboxamido)-3,3-dimethylbutanoyl)-4-hydroxy-N-(2-hydroxy-4-(4-methylthiazol-5-yl)benzyl)pyrrolidine-2-carboxamide (60 mg, 95% yield) as a yellow solid.

LC-MS (ESI): mass calcd. For C27H34F₂N405S, 564.22; m/z found, 565.3 [M+H]⁺.

Steps B-D: 2-amino-N-(S)-6-(6-(((1S,4S)-4-(2-(2-(((2S,4R)-1-(S)-2-(1-(difluoromethyl)cyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-3-ethynyl-N-methylquinoline-6-carboxamide

Example A365 was synthesized following the same procedures as described in Example A29 (5.9 mg, 24% yield over 3 steps) as a white solid. LC-MS (ESI): mass calcd. for C₆₂H₆₇F₂N₉O₉S, 1151.48 m/z found, 1152.6 [M+H]⁺. ¹H NMR (400 MHz, DMSO-d₆) δ 8.99 (s, 1H), 8.92 (s, 1H), 8.64 (s, 1H), 8.48 (d, J=7.9 Hz, 3H), 8.27 (dd, J=8.2, 2.1 Hz, 1H), 8.12-8.02 (m, 2H), 7.88 (d, J=8.6 Hz, 1H), 7.73 (s, 1H), 7.56 (d, J=7.9 Hz, 1H), 7.44-7.30 (m, 3H), 7.18 (d, J=9.5 Hz, 1H), 7.05 (d, J=1.3 Hz, 1H), 6.95 (dd, J=7.8, 1.4 Hz, 1H), 6.18 (t, J=55.5 Hz, 1H), 4.93 (s, 1H), 4.84-4.64 (m, 2H), 4.61 (d, J=9.1 Hz, 1H), 4.50 (t, J=8.2 Hz, 1H), 4.38-4.15 (m, 6H), 3.80 (s, 2H), 3.56-3.55 (m, 2H), 3.40-3.35 (m, 2H), 2.66 (s, 3H), 2.47 (s, 3H), 2.12-2.05 (m, 2H), 2.02-1.80 (m, 4H), 1.57-1.46 (m, 2H), 1.34-1.21 (m, 4H), 1.11-1.03 (m, 2H), 0.93 (s, 9H).

### Example A366. 4-amino-N-((S)-6-(6-(((1R,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-N,1-dimethyl-1H-pyrazolo[4,3-c]quinoline-8-carboxamide

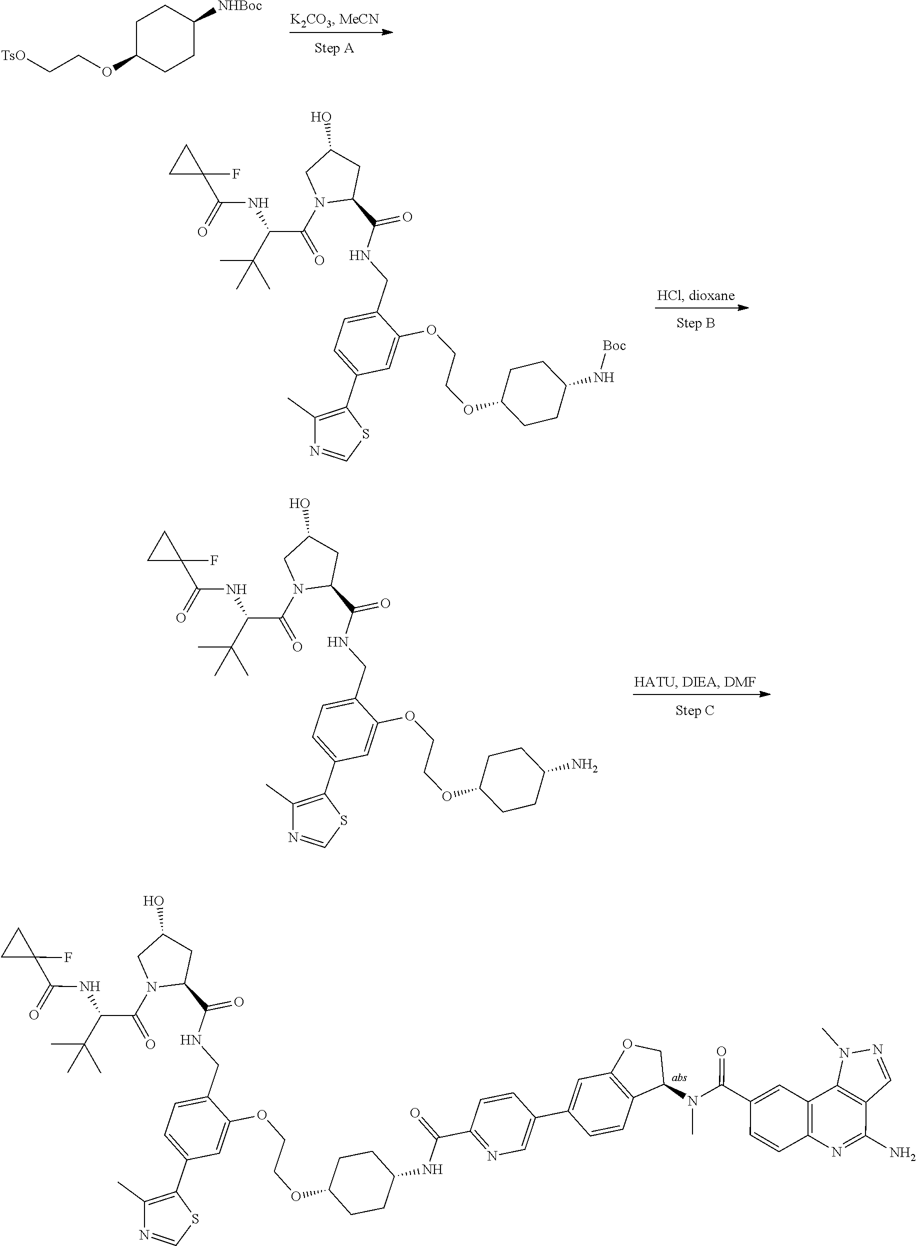 [SMILES: Cc1ccc(S(=O)(=O)OCCO[C@H]2CC[C@@H](NC(=O)OC(C)(C)C)CC2)cc1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@@H](NC(=O)OC(C)(C)C)CC2)c1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@@H](N)CC2)c1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@@H](NC(=O)c3ccc(-c4ccc5c(c4)OC[C@H]5N(C)C(=O)c4ccc5nc(N)c6cnn(C)c6c5c4)cn3)CC2)c1]

Example A366 was synthesized following the same procedures as described in Example A29 (4.4 mg, 2% yield over 3 steps) as a white solid. LC-MS (ESI): mass calcd. for C₆₁H₆₈FN₁₁O₉S, 1149.5/z found, 1151.0 [M+H]⁺. ¹H NMR (400 MHz, DMSO-d₆) δ 9.67 (s, 1H), 8.98 (s, 1H), 8.93 (s, 2H), 8.64 (s, 1H), 8.52-8.44 (m, 2H), 8.38-8.31 (m, 1H), 8.30-8.26 (m, 1H), 8.09-8.06 (m, 1H), 7.95-7.92 (m, 1H), 7.90-7.84 (m, 1H), 7.64-7.58 (m, 1H), 7.44-7.39 (m, 2H), 7.37-7.33 (m, 1H), 7.27 (d, J=8.1 Hz, 1H), 7.08 (s, 1H), 6.98 (d, J=7.7 Hz, 1H), 4.84-4.68 (m, 2H), 4.61-4.56 (m, 1H), 4.54-4.50 (m, 4H), 4.38-4.30 (m, 2H), 4.27-4.18 (m, 3H), 3.90-3.85 (m, 1H), 3.80-3.77 (m, 2H), 3.74-3.70 (m, 1H), 3.67-3.64 (m, 3H), 2.73-2.67 (m, 3H), 2.47 (s, 3H), 2.09-2.03 (m, 1H), 1,95-1.89 (m, 1H), 1.86-1.80 (m, 2H), 1.75-1.69 (m, 2H), 1.63-1.59 (m, 3H), 1.40-1.31 (m, 2H), 1.26-1.17 (m, 3H), 0.93 (s, 9H).

### Example A367. 2-amino-3-cyclopropyl-N-((S)-6-(6-(((1R,4R)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-N-methylquinoline-6-carboxamide

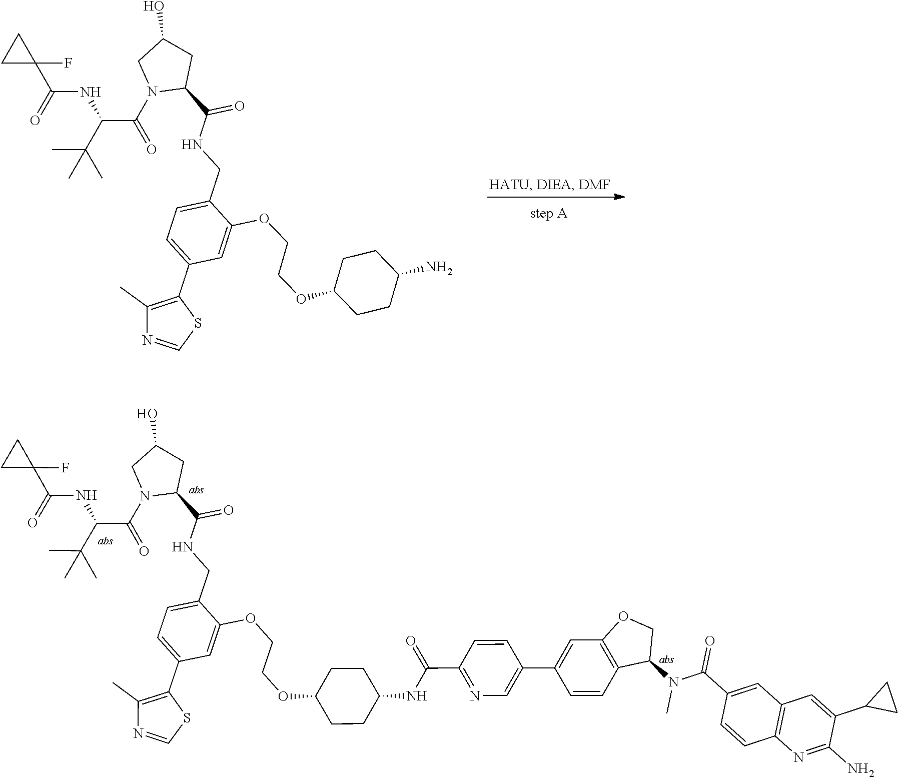 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@@H](N)CC2)c1 ; Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@@H](NC(=O)c3ccc(-c4ccc5c(c4)OC[C@H]5N(C)C(=O)c4ccc5nc(N)c(C6CC6)cc5c4)cn3)CC2)c1]

Example A367 was synthesized following the same procedures as described in Example A29 (3.6 mg, 8% yield) as a white solid. LC-MS (ESI): mass calcd, for C₆₂H7FNO₉S, 1135.5/z found, 1136.3 [M+H]⁺. ¹HNMR (400 MHz, DMSO-d₆) δ 8.98 (s, 1H), 8.93 (s, 1H), 8.50 (t, J=5.8 Hz, 1H), 8.34 (d, J=8.3 Hz, 1H), 8.27 (d, J=8.2 Hz, 1H), 8.20 (s, 1H), 8.08 (d, J=8.2 Hz, 1H), 8.04 (s, 1H), 7.85 (d, J=8.6 Hz, 1H), 7.78-7.68 (m, 1H), 7.55 (d, J=7.7 Hz, 1H), 7.45-7.37 (m, 2H), 7.34 (s, 1H), 7.27 (d, J=7.0 Hz, 1H), 7.10-7.04 (m, 1H), 7.01-6.93 (m, 1H), 4.81-4.66 (m, 2H), 4.60-4.55 (m, 1H), 4.55-4.49 (m, 1H), 4.37-4.30 (m, 2H), 4.27-4.18 (m, 3H), 3.94-3.82 (m, 2H), 3.80-3.76 (m, 2H), 3.62-3.59 (m, 3H), 2.71-2.61 (m, 3H), 2.47 (s, 3H), 2.10-2.03 (m, 1H), 1.95-1.88 (m, 2H), 1.86-1.79 (m, 2H), 1.75-1.67 (m, 2H), 1.65-1.55 (m, 3H), 1.39-1.31 (m, 2H), 1.26-1.17 (m, 3H), 1.10-1.05 (m, 2H), 0.93 (s, 9H), 0.84-0.78 (m, 2H).

### Example A368. 2-amino-3-ethynyl-N-((3S)-6-(6-(3-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)piperidin-1-yl)-8-azabicyclo[3.2.1]octane-8-carbonyl)pyridin-3-yl)-2,3-dihydrobenzofuran-3-yl)-N-methylquinoline-6-carboxamide

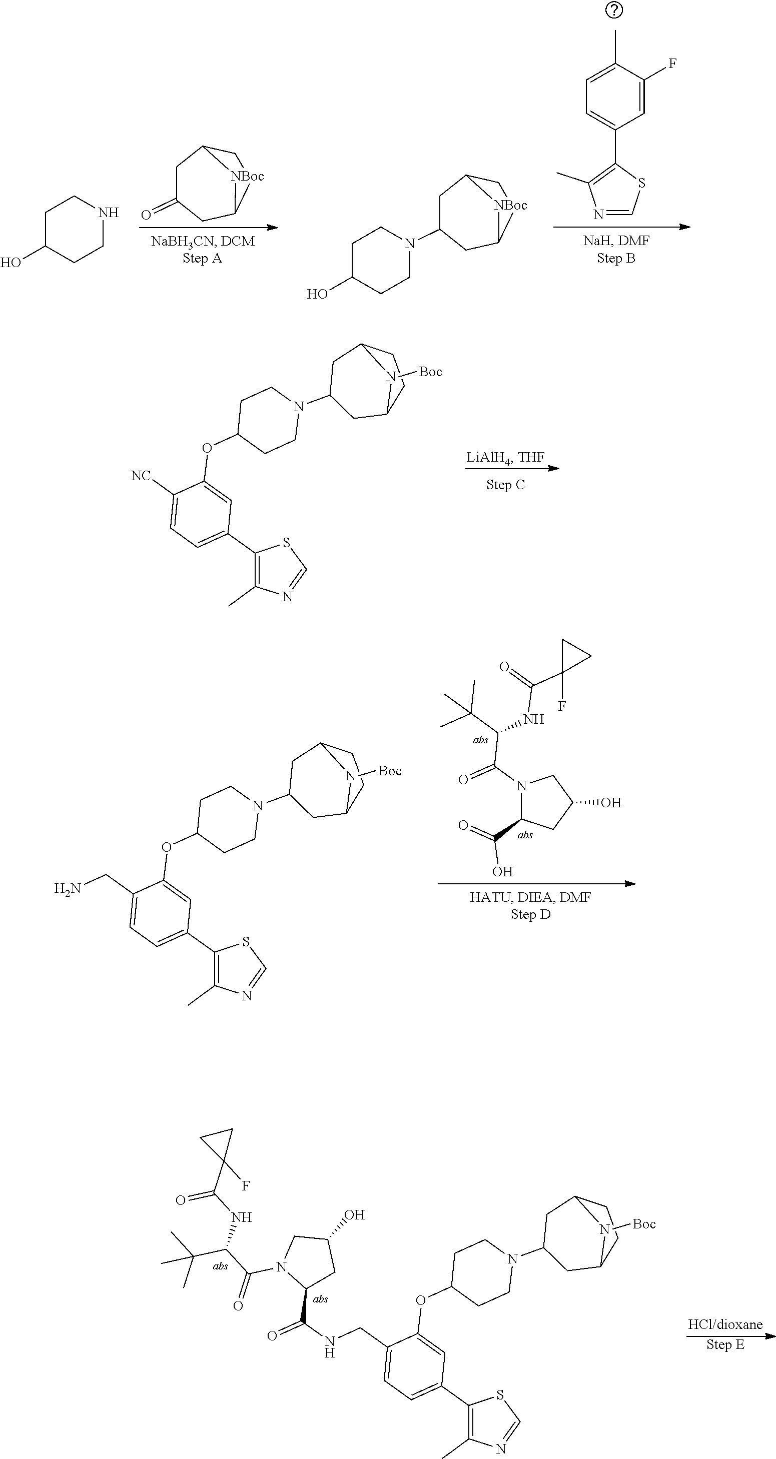

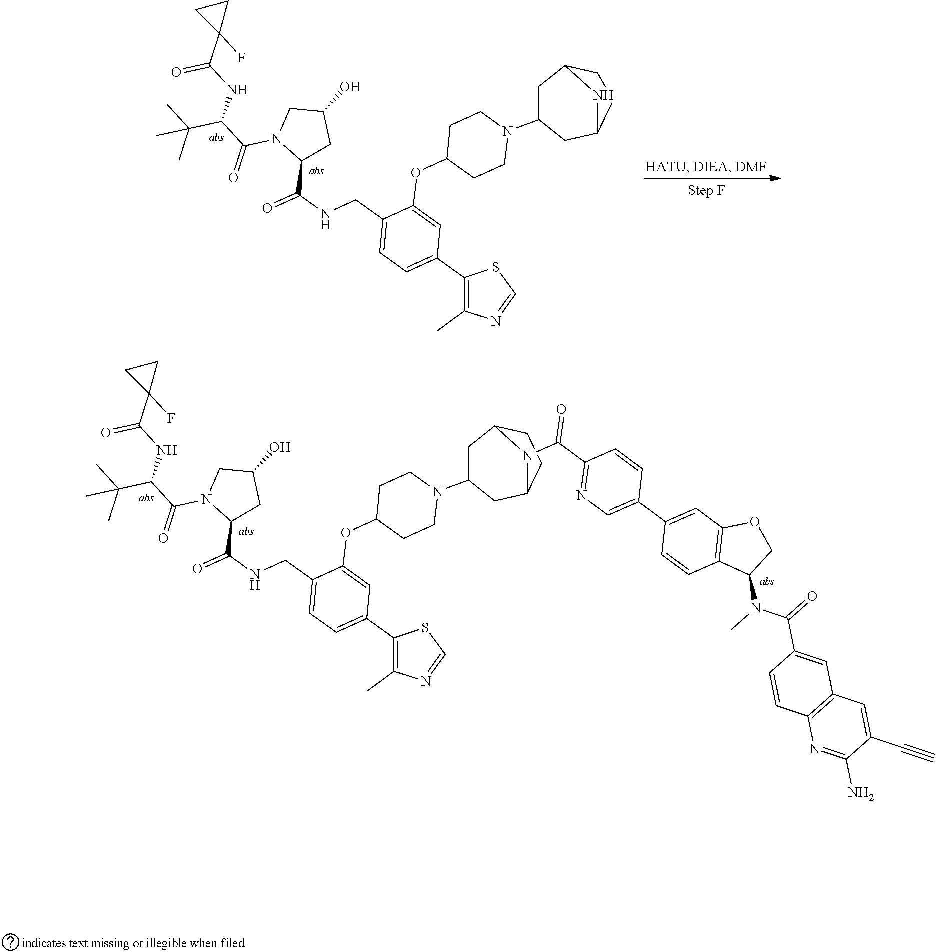 [SMILES: Cc1ncsc1-c1ccc(CNC(=O)[C@@H]2C[C@@H](O)CN2C(=O)[C@@H](NC(=O)C2(F)CC2)C(C)(C)C)c(OC2CCN(C3CC(C)NC(C)C3)CC2)c1 ; C#Cc1cc2cc(C(=O)N(C)[C@@H]3COc4cc(-c5ccc(C(=O)N6C7CCC6CC(N6CCC(Oc8cc(-c9scnc9C)ccc8CNC(=O)[C@@H]8C[C@@H](O)CN8C(=O)[C@@H](NC(=O)C8(F)CC8)C(C)(C)C)CC6)C7)nc5)ccc43)ccc2nc1N]

Step A: tert-butyl 3-(4-hydroxypiperidin-1-yl)-8-azabicyclo[3.2.1]octane-8-carboxylate

To a solution of piperidin-4-ol (1.0 g, 9.89 mmol, 1.0 eq.), tert-butyl 3-oxo-8-azabicyclo[3.2.1]octane-8-carboxylate (2.23 g, 9.89 mmol, 1.0 eq.) and acetic acid (594 mg, 9.89 mmol, 1.0 eq.) in DMF (10 mL) was stirred at 50° C. for 1 under N₂. Then the Sodium cyanoborohydride (1.24 g, 19.8 mmol, 2.0 eq.) was added, the reaction was stirred at 50° C. for 16 hours under N₂. The reaction was purified by reverse phase purification (C18, MeCN/H2O, 0.1% FA, 0 to 55% gradient, 30 min run) and the fractions containing desired product were lyophilized to afford the tert-butyl 3-(4-hydroxypiperidin-1-yl)-8-azabicyclo[3.2.1]octane-8-carboxylate (1 g, 32% yield) as a white solid. LC-MS (ESI): mass calcd, for C₁₇H₃₀N₂O₃, 310; m/z found, 311 [M+H]⁺

Step B: tert-butyl 3-(4-(2-cyano-5-(4-methylthiazol-5-yl)phenoxy)piperidin-1-yl)-8-azabicyclo[3.2.1]octane-8-carboxylate

To a stirred mixture of tert-butyl 3-(4-hydroxypiperidin-1-yl)-8-azabicyclo[3.2.1]octane-8-carboxylate (250 mg, 805 μmol, 1.0 eq.) in DMF (5 mL) were added NaH (39 mg, 1.61 mmol, 2.0 eq.) at 0° C. for 30 min, then added 2-fluoro-4-(4-methylthiazol-5-yl)benzonitrile (211 mg, 966 μmol, 1.2 eq.). The resulting mixture was stirred at room temperature for 3 hours. The reaction mixture was poured into water (20 mL), and then extracted with EtOAc (20 mL×2). The combined organic layers were washed with brine, dried over anhydrous Na₂SO₄ and concentrated. The residue was purified by column chromatography (silica gel, 0-30% of EA in PE) to afford tert-butyl 3-(4-(2-cyano-5-(4-methylthiazol-5-yl)phenoxy)piperidin-1-yl)-8-azabicyclo[3.2.1]octane-8-carboxylate (200 mg, 48% yield) as a yellow oil. LC-MS (ESI): mass calcd, for C₂₈H36N₄O₃S, 508.2; m/z found, 508.2/510.2 [M+H]⁺

Steps C-F: 2-amino-3-ethynyl-N-((3S)-6-(6-(3-(4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)piperidin-1-yl)-8-azabicyclo[3.2.1]octane-8-carbonyl)pyridin-3-yl)-2,3-dihydrobenzofuran-3-yl)-N-methylquinoline-6-carboxamide

Example A368 was synthesized following the same procedures as described in Example A23 (2.5 mg, 2% yield over 4 steps) as a white solid. LC-MS (ESI): mass calcd. for C₆₅H₇₁FN100&S, 1170.5; m/z found, 1175.4 [M+H]⁺, ¹H NMR (400 MHz, DMSO-d₆) δ 9.15 (s, 1H), 9.00 (d, J=3.4 Hz, 1H), 8.94 (s, 1H), 8.71-8.37 (m, 2H), 8.32-8.24 (m, 1H), 8.01 (s, 1H), 7.88-7.80 (m, 2H), 7.68 (s, 1H), 7.56 (d, J=7.9 Hz, 1H), 7.49-7.31 (m, 3H), 7.30-7.21 (m, 1H), 7.14-7.04 (m, 1H), 6.99 (d, J=7.8 Hz, 1H), 4.97-4.79 (m, 4H), 4.75-4.47 (m, 4H), 4.39-4.33 (m, 2H), 4.30-4.18 (m, 1H), 3.26-3.07 (m, 4H), 2.67 (s, 3H), 2.45 (s, 3H), 2.33-1.79 (m, 15H), 1.42-1.15 (m, 6H), 1.00-0.89 (m, 9H).

### Example A369. 2-amino-3-cyano-N-((S)-6-(6-((4-(2-(2-(((28,4R)-1-((S)-2-cyclopentyl-2-(1-fluorocyclopropane-1-carboxamido) acetyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

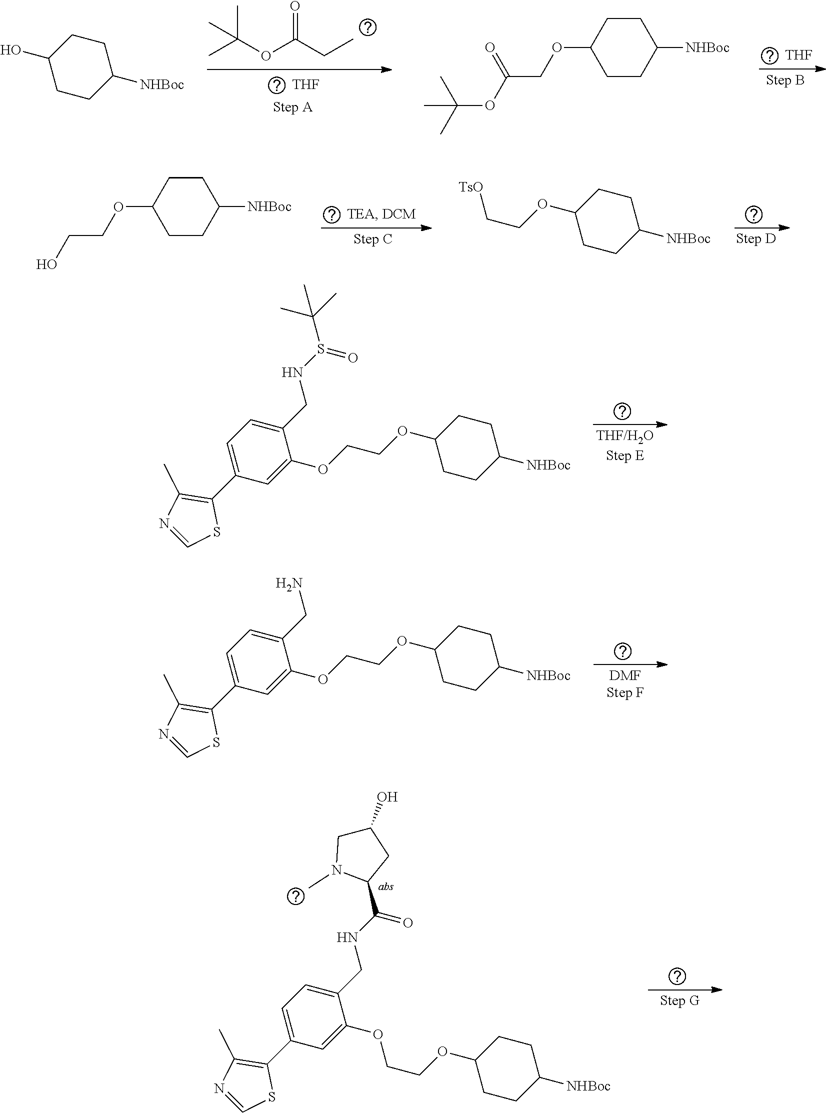

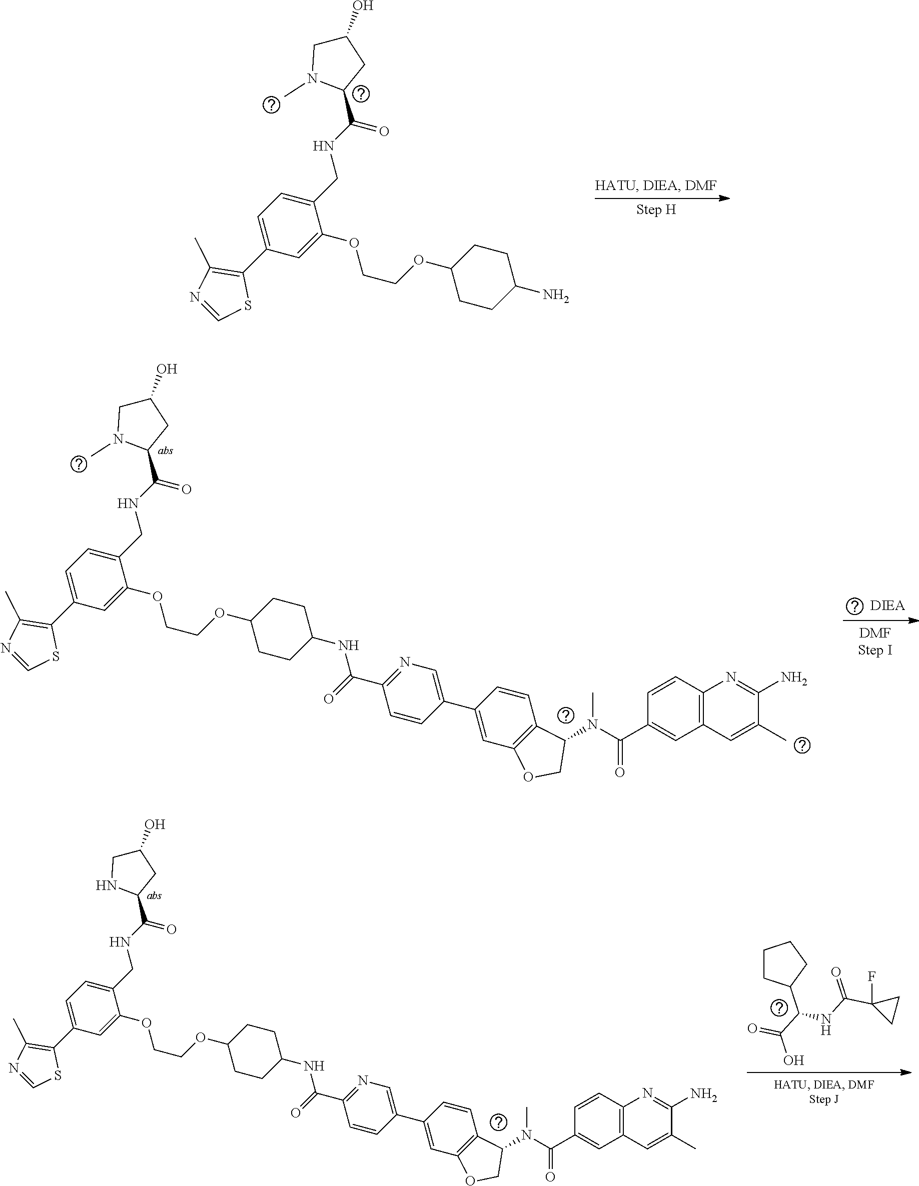 [SMILES: Cc1ncsc1-c1ccc(CNC(=O)[C@@H]2C[C@@H](O)CN2C)c(OCCOC2CCC(N)CC2)c1 ; Cc1cc2cc(C(=O)N(C)[C@@H]3COc4cc(-c5ccc(C(=O)NC6CCC(OCCOc7cc(-c8scnc8C)ccc7CNC(=O)[C@@H]7C[C@@H](O)CN7C)CC6)nc5)ccc43)ccc2nc1N ; Cc1cc2cc(C(=O)N(C)[C@@H]3COc4cc(-c5ccc(C(=O)NC6CCC(OCCOc7cc(-c8scnc8C)ccc7CNC(=O)[C@@H]7C[C@@H](O)CN7)CC6)nc5)ccc43)ccc2nc1N ; O=C(O)[C@@H](NC(=O)C1(F)CC1)C1CCCC1]

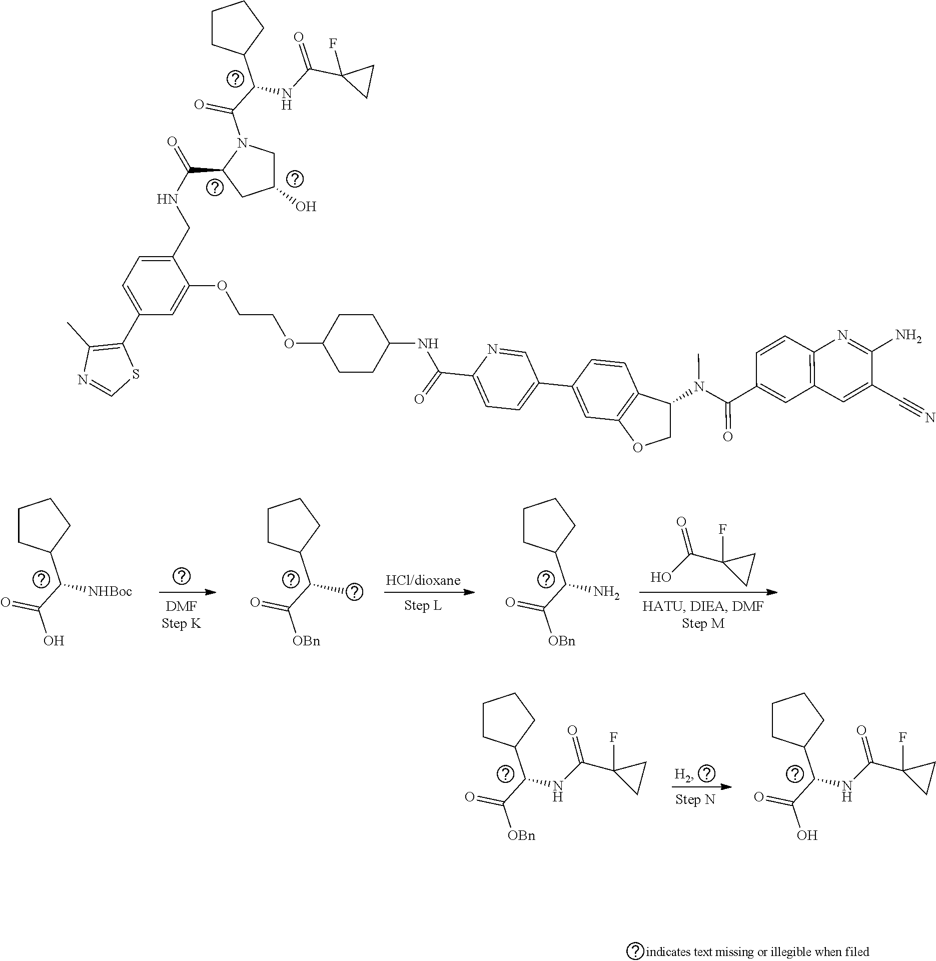

Step K: benzyl(S)-2-(tert-butoxycarbonyl)amino)-2-cyclopentylacetate

To a solution of(S)-2-((tert-butoxycarbonyl)amino)-2-cyclopentylacetic acid (500 mg, 2.06 mmol, 1.0 eq.), (bromomethyl)benzene (422 mg, 2.47 mmol, 1.2 eq.) and K₂CO₃ (426 mg, 3.08 mmol, 1.5 eq.) in DMF (10.0 mL) was stirred at room temperature for 16 hours. The reaction was quenched with water (20.0 mL), extracted with EtOAc (30.0 mL×3), the organic phase was dried over Na₂SO₄, filtered and concentrated in vacuo. The residue was purified by silica gel chromatography (100-200 mesh silica gel, 0-30% EtOAc in PE) to give benzyl(S)-2-((tert-butoxycarbonyl)amino)-2-cyclopentylacetate (300 mg, 44% yield) as a yellow oil. LC-MS (ESI): mass calcd. For C₁₉H₂₇NO₄, 333.2; m/z found, 334 [M+H]⁺.

Step L: benzyl(S)-2-amino-2-cyclopentylacetate

To a solution of benzyl(S)-2-((tert-butoxycarbonyl)amino)-2-cyclopentylacetate (300 mg, 900 μmol, 1.0 eq.) in DCM (3.0 mL) was added HCl in dioxane (3.0 mL). The mixture was stirred at room temperature for 30 mins. The reaction solution was concentrated in vacuo to give the benzyl(S)-2-amino-2-cyclopentylacetate (209 mg, 99% yield) as a yellow solid. LC-MS (ESI): mass calcd. For C₁₄H₁₉NO₂, 233.2; m/z found, 234 [M+H]⁺.

Step M: benzyl(S)-2-cyclopentyl-2-(1-fluorocyclopropane-1-carboxamido)acetate

To a mixture of 1-fluorocyclopropane-1-carboxylic acid (100 mg, 961 μmol, 1.0 eq.), HATU (548 mg, 1.44 mmol, 1.5 eq.) and DIEA (373 mg, 2.88 mmol, 3.0 eq.) in DMF (5.0 mL) was added benzyl(S)-2-amino-2-cyclopentylacetate (224 mg, 961 μmol, 1.0 eq.). The mixture was stirred at 0° C. for 30 mins under N₂ atmosphere. The resulting solution was diluted with 10 mL of water and extracted with EtOAc (20 mL×3). The combined organic layers were washed with brine, dried over anhydrous Na₂SO₄ and concentrated under reduced pressure. The residue was purified by flash column chromatography on silica gel (100-200 mesh silica gel, 0-20% EtOAc in PE) to afford benzyl(S)-2-cyclopentyl-2-(1-fluorocyclopropane-1-carboxamido)acetate (280 mg, 91% yield) as a yellow solid. LC-MS (ESI): mass calcd. For C₁₈H₂₂FNO₃, 319.2; m/z found, 320 [M+H]⁺.

Step N: (S)-2-cyclopentyl-2-(1-fluorocyclopropane-1-carboxamido) acetic acid

To the solution of benzyl(S)-2-cyclopentyl-2-(1-fluorocyclopropane-1-carboxamido)acetate (280 mg, 877 μmol, 1.0 eq) in MeOH (10.0 mL) were added Pd/C (150 mg, 1.41 mmol, 1,5 eq) and stirred for 15 hours at room temperature. The residue was concentrated in vacuo to give the(S)-2-cyclopentyl-2-(1-fluorocyclopropane-1-carboxamido) acetic acid (200 mg, 99% yield) as a yellow oil. LC-MS (ESI): mass calcd. For C₁₁H₁₆FNO₃, 229.2; m/z found, 230 [M+H]⁺.

Steps A-J: 2-amino-3-cyano-N-(S)-6-16-((4-(2-(2-(((25, 4R)-1-(S)-2-cyclopentyl-2-(1-fluorocyclopropane-1-carboxamido) acetyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

Example A369 was synthesized following the same procedures as described in Example A373 (6 mg, 1% yield over 10 steps) as a yellow solid. LC-MS (ESI): mass calcd. for C₆₁H₆₈FN₁₀O₇S, 1132.5; m/z found, 1134 [M+H]⁺. ¹H NMR (400 MHz, DMSO-d₆) δ 9.00 (s, 1H), 8.92 (s, 1H), 8.83 (s, 1H), 8.49 (d, J=8.4 Hz, 1H), 8.33 (t, J=5.9 Hz, 1H), 8.28 (dd, J=8.2, 2.0 Hz, 1H), 8.07 (t, J=7.4 Hz, 2H), 7.98 (s, 1H), 7.82 (dd, J=8.7, 1.7 Hz, 1H), 7.60 (d, J=8.5 Hz, 1H), 7.56 (d, J=7.7 Hz, 1H), 7.51-7.45 (m, 1H), 7.39 (d, J=7.6 Hz, 1H), 7.34 (d, J=7.4 Hz, 2H), 7.06 (s, 1H), 6.99 (d, J=7.8 Hz, 1H), 4.69 (m, 2H), 4.50 (m, 3H), 4.37 (m, 1H), 4.28 (d, J=5.7 Hz, 2H), 4.18 (s, 2H), 3.72-3.61 (m, 2H), 3.44-3.33 (m, 1H), 3.36 (m, 1H), 2.67 (s, 3H), 2.48 (s, 3H), 2.37-2.26 (m, 1H), 2.08-2.02 (m, 3H), 1.99-1.95 (m, 1H), 1.89-1.83 (m, 2H), 1.73-1.61 (m, 2H), 1.55-1.48 (m, 4H), 1.45-1.38 (m, 2H), 1.35-1.28 (m, 4H), 1.24 (s, 2H), 1.20-1.17 (m, 2H).

### Example A370. 2-amino-3-ethynyl-N-((3S)-6-(6-(((1R,3S,5R)-5-(2-(2-(((28,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) adamantan-2-yl)carbamoyl)pyridin-3-yl)-2,3-dihydrobenzofuran-3-yl)-N-methylquinoline-6-carboxamide

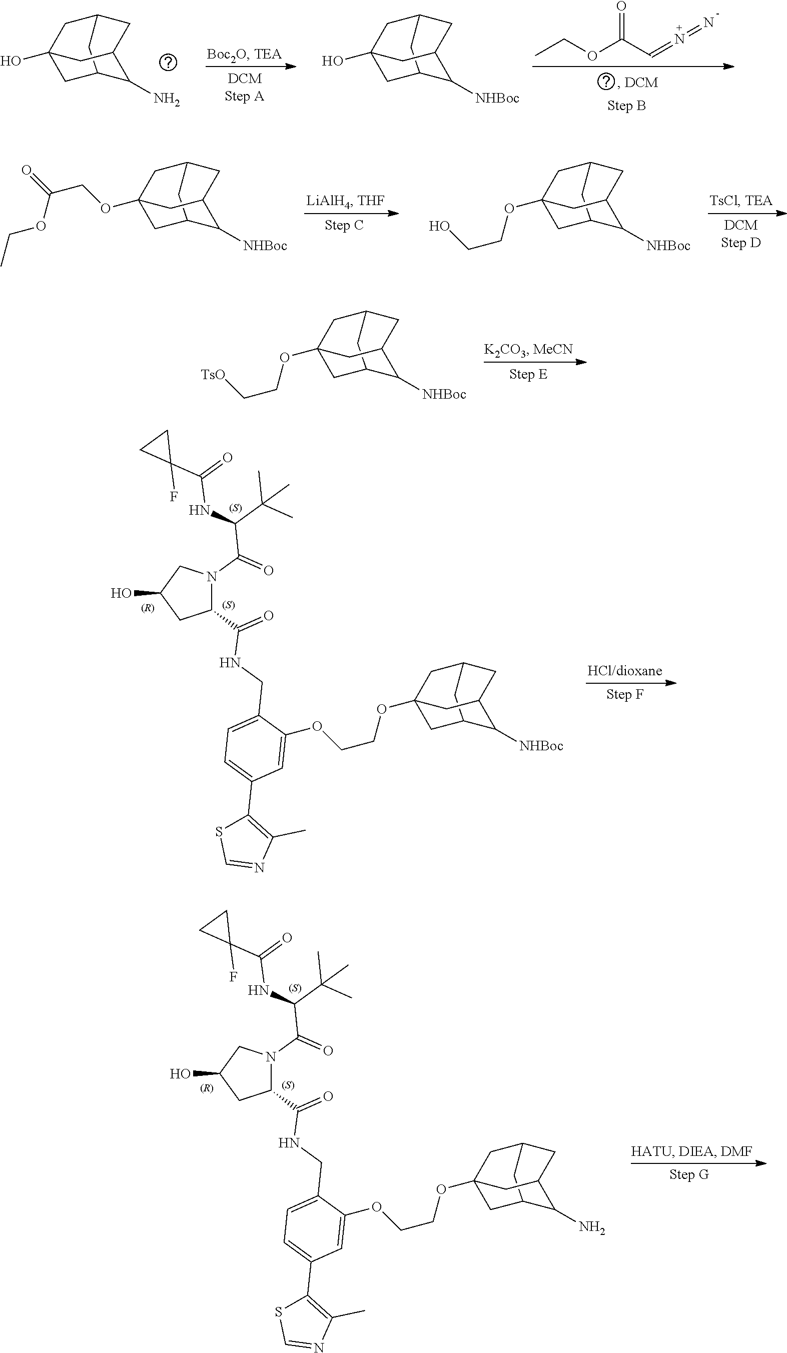

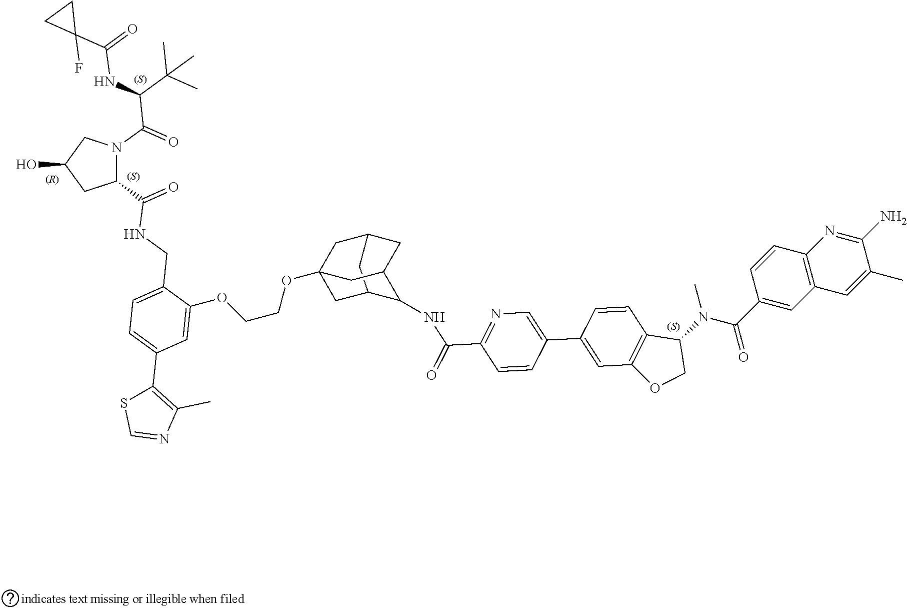

Step A: tert-butyl ((1R,3S,5s)-5-hydroxyadamantan-2-yl)carbamate

To a mixture of (1s,3R,5S)-4-aminoadamantan-1-ol (1.00 g, 5.98 mmol, 1.0 eq.), Di-tert-butyl dicarbonate (1.57 g, 7.17 mmol, 1.2 eq.) in DCM (20 mL) was added TEA (1.82 g, 17.9 mmol, 3.0 eq.). The mixture was stirred at room temperature for 12 hours under N₂ atmosphere. The resulting solution was diluted with 10 mL of water and extracted with EtOAc (30×3 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford tert-butyl ((1R,3S,5s)-5-hydroxyadamantan-2-yl)carbamate (800 mg, 50% yield) as a white oil. LC-MS (ESI): mass calcd. For C₁₅H25NO_{3, 267.18}; m/z found, 266.1 [M−H]⁻.

Step B: ethyl 2-(((1s, 3R,5S)-4-((tert-butoxycarbonyl)amino) adamantan-1-yl)oxy)acetate

To a mixture of tert-butyl ((1R,3S,5s)-5-hydroxyadamantan-2-yl)carbamate (100 mg, 374 μmol, 1.0 eq.), Rhodium (II) AcetateDimer (16 mg, 37.4 μmol, 0.1 eq.) in DCM (3 mL) at 0° C. was added ethyl 2-diazoacetate (128 mg, 1.12 mmol, 3.0 eq.). The mixture was stirred at room temperature for 12 hours under N₂ atmosphere. The resulting solution was diluted with 10 mL of water and extracted with EtOAc (30×3 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford ethyl 2-(((1s,3R,5S)-4-((tert-butoxycarbonyl)amino) adamantan-1-yl)oxy)acetate (60.0 mg, 45% yield) as a white oil. LC-MS (ESI): mass calcd. For C₁₉H₃₁NO₅, 353.22; m/z found, 352.1 [M−H]⁻.

Step C: tert-butyl ((1R,3S,5s)-5-(2-hydroxyethoxy) adamantan-2-yl)carbamate

To a mixture of ethyl 2-(((1s,3R,5S)-4-((tert-butoxycarbonyl)amino) adamantan-1-yl)oxy)acetate (300 mg, 849 μmol, 1.0 eq.) in THF (10 mL) was added Aluminum lithium hydride (64 mg, 1.70 mmol, 2.0 eq.) at 0° C. The mixture was stirred at room temperature for 1 hour under N2 atmosphere. The resulting solution was diluted with 10 mL of Potassium sodium tartrate in water and extracted with EtOAc (30×3 mL). The combined organic layers were washed with brine, dried over anhydrous Na₂SO₄ and concentrated under reduced pressure to afford tert-butyl ((1R,3S,5s)-5-(2-hydroxyethoxy) adamantan-2-yl)carbamate (160 mg, 61% yield) as a yellow oil, which was used directly without further purification. LC-MS (ESI): mass calcd. For C₁₇H₂₉NO₄, 311.21; m/z found, 310.1 [M−H]⁻.

Step D: 2-(((1s, 3R,5S)-4-((tert-butoxycarbonyl)amino) adamantan-1-yl)oxy)ethyl 4-methylbenzenesulfonate

To a mixture of tert-butyl ((1R,3S,5s)-5-(2-hydroxyethoxy) adamantan-2-yl)carbamate (160 mg, 514 μmol, 1.0 eq), p-Toluenesulfonyl chloride (147 mg, 771 μmol, 1.5 eq) in DCM (10 mL) was added TEA (52 mg, 514 μmol, 1.0 eq). The mixture was stirred at room temperature for 2 hours under N₂ atmosphere. The resulting solution was diluted with 10 mL of water and extracted with EtOAc (30×3 mL). The combined organic layers were washed with brine, dried over anhydrous Na₂SO₄ and concentrated under reduced pressure. The residue was purified by flash column chromatography on silica gel (100-200 mesh silica gel, 0-50% EtOAc in PE) to afford 2-(((1s,3R,5S)-4-((tert-butoxycarbonyl)amino) adamantan-1-yl)oxy)ethyl 4-methylbenzenesulfonate (200 mg, 84% yield) as a yellow oil. LC-MS (ESI): mass calcd. For C₂₄H35NO₆S, 465.22; m/z found, 464.3 [M−H]⁺.

Steps E-G: 2-amino-3-ethynyl-N-(3S)-6-(6-(1R,35,5R)-5-(2-(2-((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) adamantan-2-yl)carbamoyl)pyridin-3-yl)-2,3-dihydrobenzofuran-3-yl)-N-methylquinoline-6-carboxamide

Example A370 was synthesized following the same procedures as described in Example A29 (3 mg, 5% yield over 3 steps) as a white solid. LC-MS (ESI): mass calcd, for C₆₅H₇₀FN909S, 1171.50 m/z found, 1172.5 [M+H]⁺. ¹H NMR (400 MHz, DMSO-d₆) δ 8.98 (d, J=8.1 Hz, 2H), 8.68 (s, 1H), 8.61 (d, J=9.4 Hz, 1H), 8.49 (t, J=5.6 Hz, 1H), 8.36 (d, J=7.7 Hz, 1H), 8.30 (d, J=8.0 Hz, 1H), 8.13-8.03 (m, 2H), 7.91 (d, J=8.7 Hz, 1H), 7.76 (s, 1H), 7.56 (d, J=7.8 Hz, 1H), 7.45-7.34 (m, 3H), 7.29 (d, J=6.8 Hz, 1H), 7.05 (s, 1H), 6.97 (d, J=7.4 Hz, 1H), 6.00 (d, J=312.3 Hz, 1H), 4.96 (s, 1H), 4.89-4.65 (m, 2H), 4.59 (d, J=9.8 Hz, 1H), 4.52 (t, J=8.3 Hz, 1H), 4.38-4.21 (m, 5H), 4.14 (s, 2H), 4.04 (s, 2H), 3.62-3.58 (m, 2H), 2.66 (s, 3H), 2.47 (s, 3H), 2.26-2.10 (m, 4H), 2.03-1.93 (m, 2H), 1.86-1.79 (m, 4H), 1.73 (s, 1H), 1.53 (d, J=11.0 Hz, 2H), 1.40-1.33 (m, 2H), 1.29-1.17 (m, 4H), 0.95 (s, 9H).

### Example A371. 2-amino-3-cyano-N-((S)-6-(6-((4-(2-(2-(((2S,4R)-1-((S)-2-(1-fluorocyclopropane-1-carboxamido)-2-phenylacetyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methylquinoline-6-carboxamide

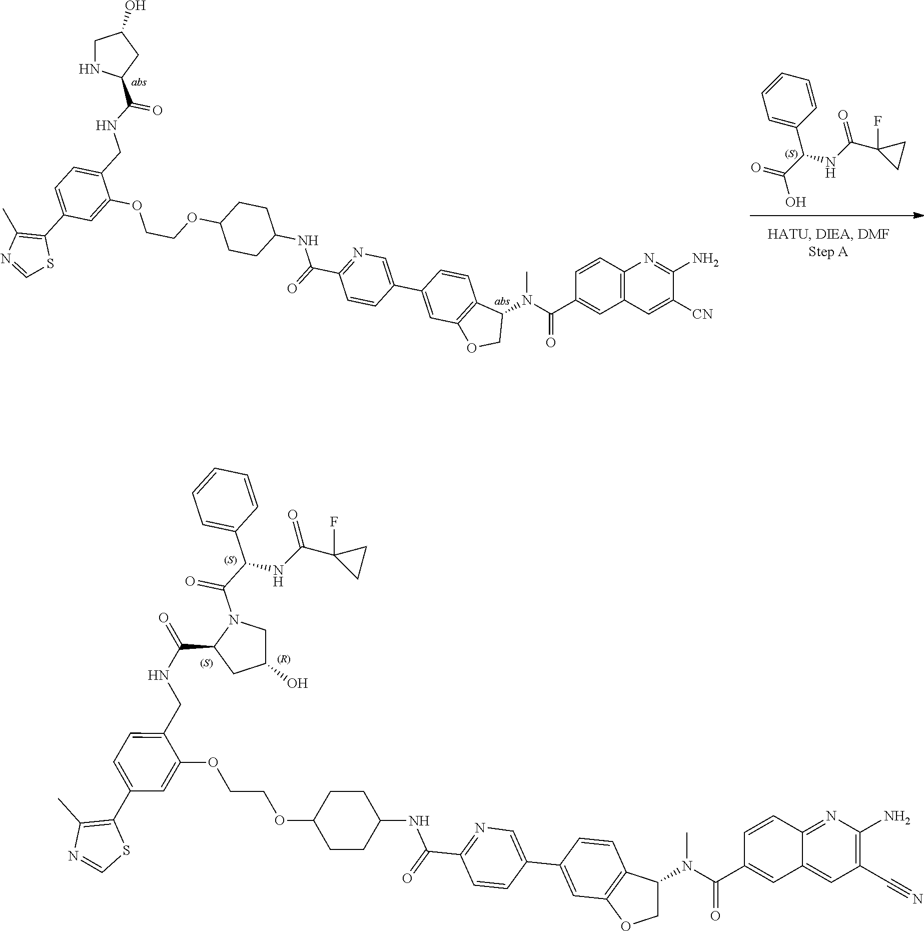

Example A371 was synthesized following the same procedures as described in Example A373 (1.1 mg, 9% yield) as a white solid. LC-MS (ESI): mass calcd, for C₆₂H₆₁FN₁₀O₉S, 1140.43; m/z found, 1141.5 [M+H]⁺. ¹H NMR (400 MHz, DMSO-d₆) δ 9.00 (s, 1H), 8.90 (s, 1H), 8.85 (d, J=12.0 Hz, 1H), 8.49 (d, J=8.4 Hz, 1H), 8.45-8.30 (m, 2H), 8.27 (dd, J=8.1, 2.1 Hz, 1H), 8.08 (d, J=8.2 Hz, 1H), 7.98 (s, 1H), 7.83 (dd, J=8.7, 1.8 Hz, 1H), 7.60 (d, J=7.8 Hz, 1H), 7.56 (d, J=7.8 Hz, 1H), 7.44 (d, J=6.8 Hz, 2H), 7.37 (dd, J=8.2, 3.2 Hz, 3H), 7.34-7.27 (m, 5H), 7.08-7.02 (m, 2H), 5.76 (d, J=7.5 Hz, 1H), 5.52-5.26 (m, 1H), 4.84-4.72 (m, 1H), 4.71-4.67 (m, 1H), 4.55 (t, J=7.9 Hz, 1H), 4.34 (d, J=10.4 Hz, 1H), 4.29-4.24 (m, 2H), 4.18 (s, 2H), 3.84-3.80 (m, 2H), 3.62 (s, 1H), 3.53 (d, J=11.1 Hz, 1H), 3.37 (t, J=10.2 Hz, 1H), 3.04 (dd, J=10.7, 4.0 Hz, 1H), 2.67 (s, 3H), 2.48 (s, 3H), 2.07-2.03 (m, 2H), 1.87-1.83 (m, 2H), 1.56-1.46 (m, 2H), 1.35-1.31 (m, 2H), 1.30-1.26 (m, 2H), 1.23 (s, 2H), 1.20-1.14 (m, 1H), 1.10-0.90 (m, 1H).

### Example A373. 2-amino-3-cyclopropyl-N-((S)-6-(6-(4-((4-fluoro-1~(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

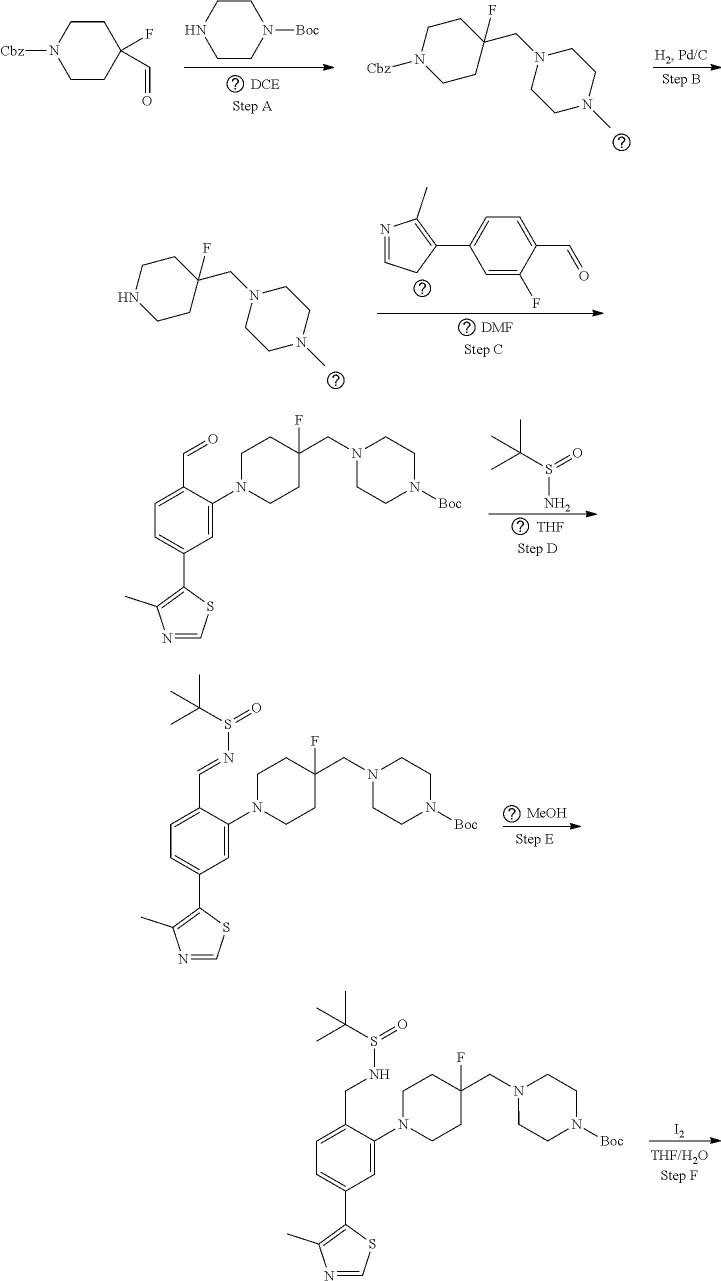

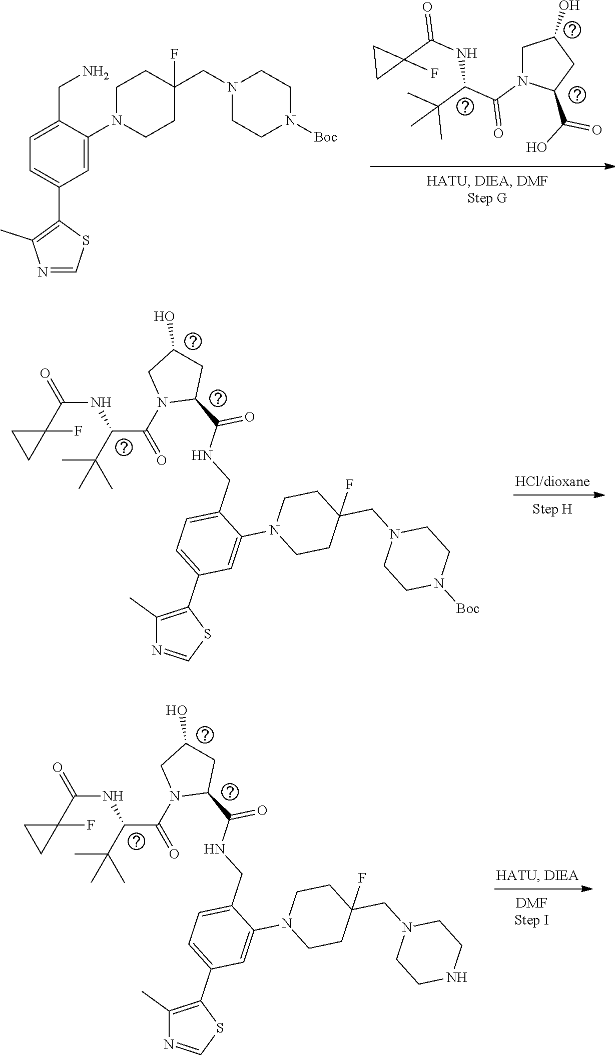 [SMILES: Cc1ncsc1-c1ccc(CN)c(N2CCC(F)(CN3CCN(C(=O)OC(C)(C)C)CC3)CC2)c1 ; CC(C)(C)[C@H](NC(=O)C1(F)CC1)C(=O)N1C[C@H](O)C[C@H]1C(=O)O ; Cc1ncsc1-c1ccc(CNC(=O)[C@@H]2C[C@@H](O)CN2C(=O)[C@@H](NC(=O)C2(F)CC2)C(C)(C)C)c(N2CCC(F)(CN3CCN(C(=O)OC(C)(C)C)CC3)CC2)c1 ; Cc1ncsc1-c1ccc(CNC(=O)[C@@H]2C[C@@H](O)CN2C(=O)[C@@H](NC(=O)C2(F)CC2)C(C)(C)C)c(N2CCC(F)(CN3CCNCC3)CC2)c1]

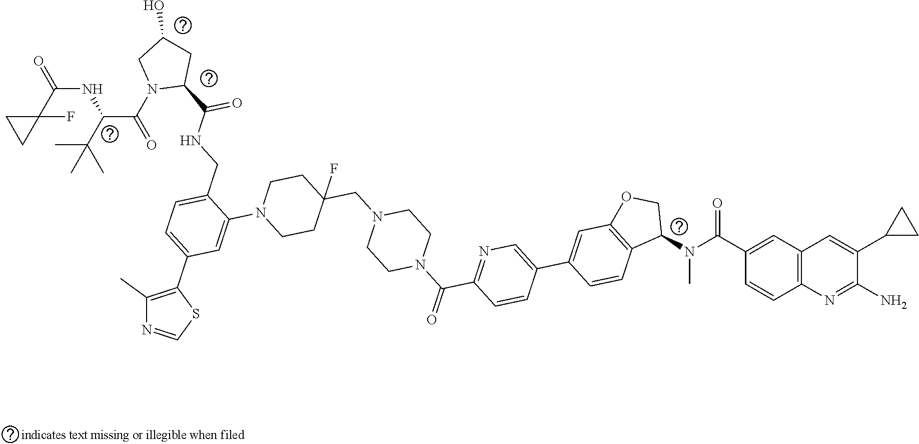 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(F)(CN3CCN(C(=O)c4ccc(-c5ccc6c(c5)OC[C@H]6N(C)C(=O)c5ccc6nc(N)c(C7CC7)cc6c5)cn4)CC3)CC2)c1]

Step A: tert-butyl 4-((1-((benzyloxy)carbonyl)-4-fluoropiperidin-4-yl)methyl)piperazine-1-carboxylate

A mixture of benzyl 4-fluoro-4-formylpiperidine-1-carboxylate (10 g, 38 mmol, 1.0 eq.) (WO2023/220640, 2023, A1), tert-butyl piperazine-1-carboxylate (14 g, 75.4 mmol, 2.0 eq.) and HOAC (2.3 g, 37.7 mmol, 1.0 eq.) in DCM (30 mL) was stirred at room temperature for 30 mins under nitrogen. Then NaBH (OAc) 3 (16 g, 75.4 mmol, 2.0 eq.) was added and stirred at room temperature for 16 hours. The resulting solution was diluted with 100 mL of water and extracted with EtOAc (100 mL×3). The combined organic layers were washed with brine (200 mL), dried over Na₂SO₄ and concentrated under reduced pressure. The crude product was purified by flash column chromatography on silica gel (100-200 mesh silica gel, 0~40% EtOAc in PE) to afford tert-butyl 4-((1-((benzyloxy)carbonyl)-4-fluoropiperidin-4-yl)methyl)piperazine-1-carboxylate (8 g, 48% yield) as a yellow solid. LC-MS (ESI): mass calcd. For C₂₃H34FN304, 435.25; m/z found, 436 [M+H]⁺.

Step B: tert-butyl ((S)-6-(2-((1S,4S)-4-(2-(2-(((2S, 4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1-oxo-1,2,3,4-tetrahydroisoquinolin-6-yl)-2,3-dihydrobenzofuran-3-yl)(methyl)carbamate

A mixture of tert-butyl 4-((1-((benzyloxy)carbonyl)-4-fluoropiperidin-4-yl)methyl)piperazine-1-carboxylate (6 g, 13.8 mmol, 1.0 eq.), Pd/C (1.0 g) and Pd (OH) 2 (1.0 g) in MeOH (40 mL) was stirred at 50° C. for 16 hours under H₂. After cooling to room temperature, the resulting solution was filtered and concentrated under reduced pressure to afford tert-butyl 4-((4-fluoropiperidin-4-yl)methyl)piperazine-1-carboxylate (4.1 g, 100% yield) as a white solid.

LC-MS (ESI): mass calcd. For C₁₅H28FN302, 301.22; m/z found, 302 [M+H]⁺.

Steps F-I: 2-amino-3-cyclopropyl-N-((S)-6-(6-(4-((4-fluoro-1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

Example A373 was synthesized following the same procedures as described in Example A126 (3 mg, 2% yield over 7 steps) as a white solid. LC-MS (ESI): mass calcd, for C₆₄H₆₉FN₈O₉S, 1144.49; m/z found, 1145.8 [M+H]⁺. ¹H NMR (400 MHz, DMSO-d₆) δ 8.99 (s, 1H), 8.93 (s, 1H), 8.82 (s, 2H), 8.52 (s, 1H), 8.28-8.20 (m, 2H), 8.05 (s, 1H), 7.86 (d, J=8.4 Hz, 1H), 7.79-7.71 (m, 2H), 7.55 (d, J=7.6 Hz, 1H), 7.45 (d, J=8.0 Hz, 1H), 7.42-7.38 (m, 1H), 7.35 (s, 1H), 7.27 (d, J=7.5 Hz, 1H), 7.18-7.13 (m, 2H), 4.84-4.66 (m, 2H), 4.60 (d, J=9.3 Hz, 1H), 4.52-4.47 (m, 1H), 4.46-4.24 (m, 4H), 3.68-3.62 (m, 8H), 3.00-2.89 (m, 4H), 2.67 (s, 3H), 2.46 (s, 3H), 2.12-1.88 (m, 9H), 1.40-1.32 (m, 2H), 1.26-1.19 (m, 4H), 1.10-1.06 (m, 2H), 0.99-0.95 (m, 9H), 0.84-0.80 (m, 2H).

### Example A374. 2-amino-N-((S)-6-(6-(((1S,4S)-4-(2-(5-cyano-2-(((2S,4R)-1-((S)-2-(1-fluorocyclopropane-1-carboxamido)-3,3-dimethylbutanoyl)-4-hydroxypyrrolidine-2-carboxamido)methyl)phenoxy)ethoxy)cyclohexyl)carbamoyl)pyridin-3-yl)-2,3-dihydrobenzofuran-3-yl)-3-cyclopropyl-N-methylquinoline-6-carboxamide

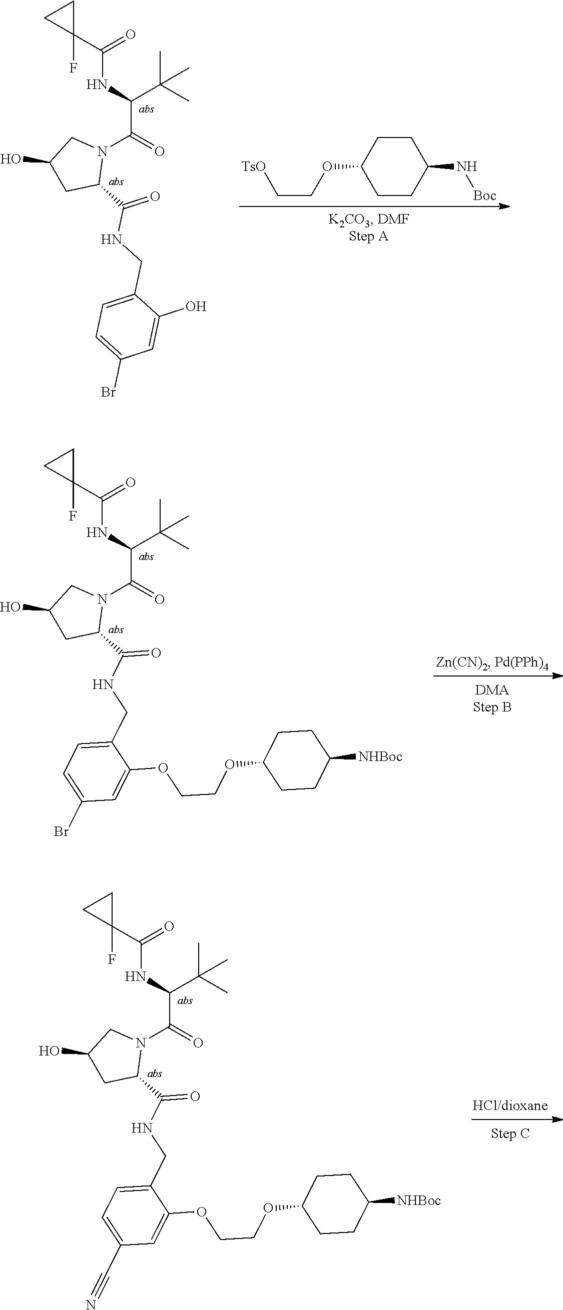 [SMILES: CC(C)(C)[C@H](NC(=O)C1(F)CC1)C(=O)N1C[C@H](O)C[C@H]1C(=O)NCc1ccc(Br)cc1O ; Cc1ccc(S(=O)(=O)OCCO[C@H]2CC[C@H](NC(=O)OC(C)(C)C)CC2)cc1 ; CC(C)(C)OC(=O)N[C@H]1CC[C@H](OCCOc2cc(Br)ccc2CNC(=O)[C@@H]2C[C@@H](O)CN2C(=O)[C@@H](NC(=O)C2(F)CC2)C(C)(C)C)CC1 ; CC(C)(C)OC(=O)N[C@H]1CC[C@H](OCCOc2cc(C#N)ccc2CNC(=O)[C@@H]2C[C@@H](O)CN2C(=O)[C@@H](NC(=O)C2(F)CC2)C(C)(C)C)CC1]

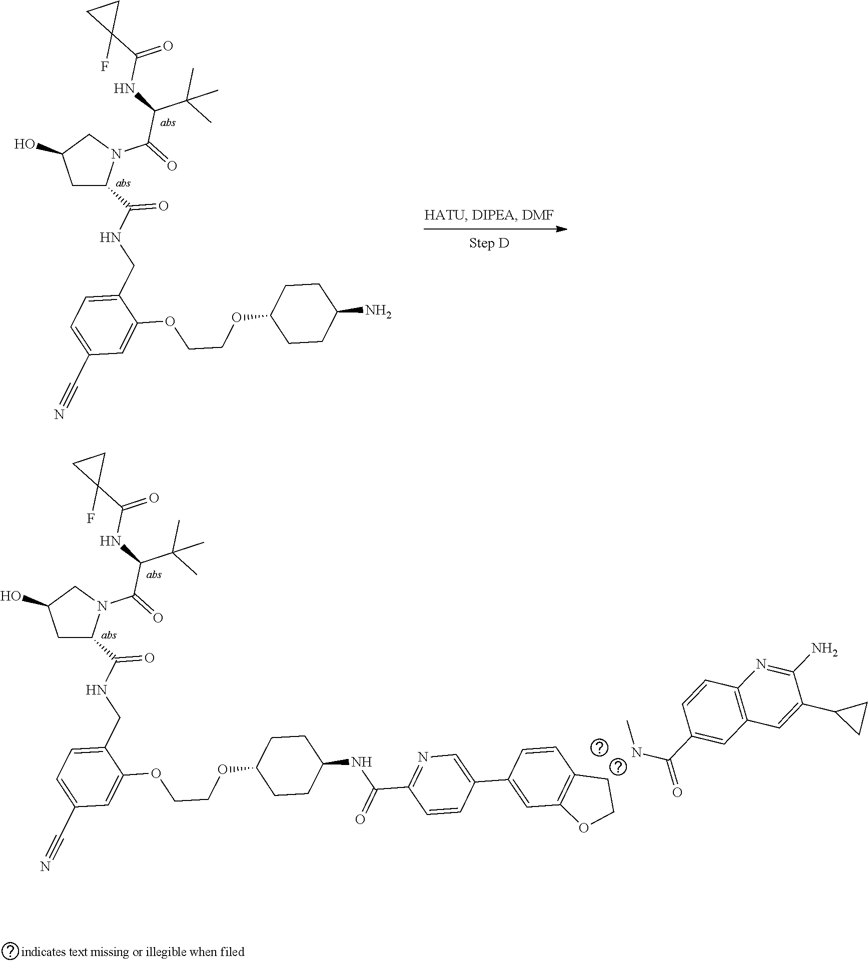

Example A374 was synthesized following the same procedures as described in Example A107 (5.7 mg, 5% yield over 4 steps) as a white solid. LC-MS (ESI): mass calcd. for C₅₉H₆₆FN₉O₉S, 1063.5/z found, 1064.7 [M+H]⁺. ¹H NMR (400 MHz, DMSO-d₆) δ 9.00-8.71 (m, 3H), 8.61 (t, J=5.8 Hz, 1H), 8.49 (d, J=8.4 Hz, 1H), 8.31-8.18 (m, 2H), 8.11-8.01 (m, 2H), 7.86 (d, J=8.5 Hz, 1H), 7.80-7.71 (m, 1H), 7.58-7.50 (m, 2H), 7.46 (s, 1H), 7.42-7.37 (m, 1H), 7.36-7.26 (m, 3H), 4.91-4.67 (m, 2H), 4.59 (d, J=9.3 Hz, 1H), 4.51 (t, J=8.3 Hz, 1H), 4.38-4.30 (m, 2H), 4.24-4.16 (m, 3H), 3.80 (s, 4H), 3.61-3.58 (m, 2H), 3.43-3.28 (m, 1H), 2.65 (s, 3H), 2.12-2.01 (m, 3H), 1.96-1.84 (m, 3H), 1.57-1.47 (m, 2H), 1.42-1.31 (m, 3H), 1.28-1.18 (m, 3H), 1.10-1.05 (m, 2H), 0.95 (s, 9H), 0.82 (d, J=3.9 Hz, 2H).

### Example A375. 2-amino-N-((S)-6-(6-(((18,4S)-4-(2-((4′-cyano-4-(((2S,4R)-1-((S)-2-(1-fluorocyclopropane-1-carboxamido)-3,3-dimethylbutanoyl)-4-hydroxypyrrolidine-2-carboxamido)methyl)-[1,1′-biphenyl]-3-yl)oxy)ethoxy)cyclohexyl)carbamoyl)pyridin-3-yl)-2,3-dihydrobenzofuran-3-yl)-3-ethynyl-N-methylquinoline-6-carboxamide

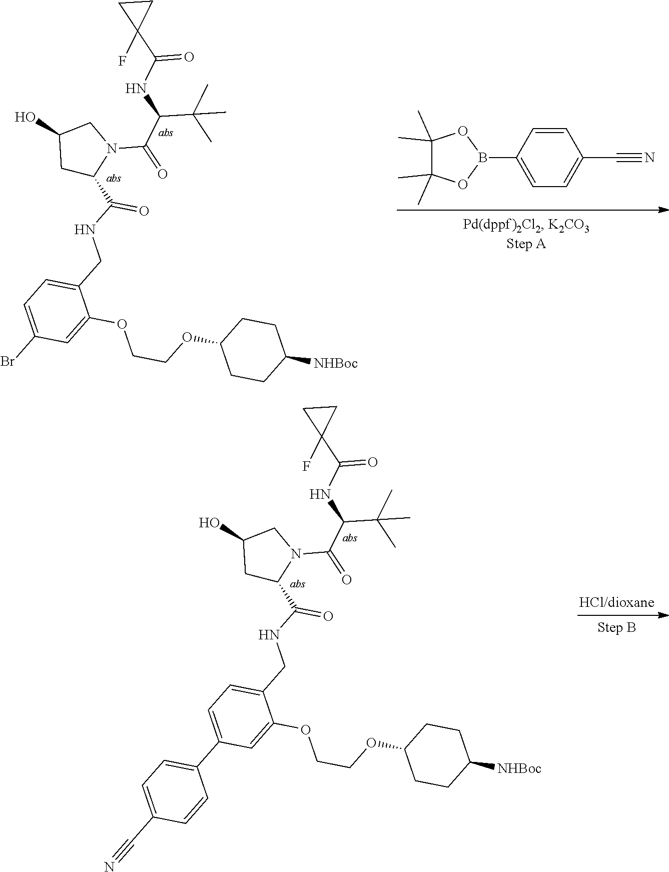 [SMILES: CC(C)(C)OC(=O)N[C@H]1CC[C@H](OCCOc2cc(Br)ccc2CNC(=O)[C@@H]2C[C@@H](O)CN2C(=O)[C@@H](NC(=O)C2(F)CC2)C(C)(C)C)CC1 ; CC1(C)OB(c2ccc(C#N)cc2)OC1(C)C ; CC(C)(C)OC(=O)N[C@H]1CC[C@H](OCCOc2cc(-c3ccc(C#N)cc3)ccc2CNC(=O)[C@@H]2C[C@@H](O)CN2C(=O)[C@@H](NC(=O)C2(F)CC2)C(C)(C)C)CC1]

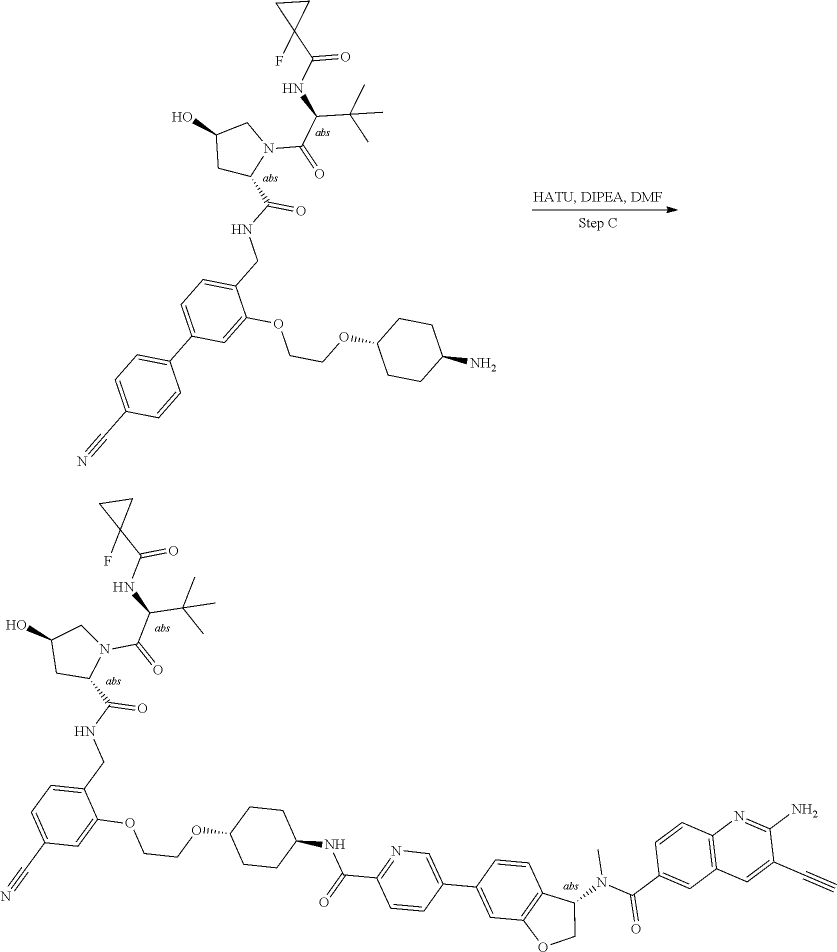

Example A375 was synthesized following the same procedures as described in Example A107 (4.1 mg, 4% yield over 3 steps) as a white solid. LC-MS (ESI): mass calcd, for C₆₄H₆₆FN₉O₉, 1123.50; m/z found, 1125.2 [M+H]⁺. ¹H NMR (400 MHz, DMSO DMSO-d₆) δ 8.94-8.91 (m, 1H), 8.61 (s, 1H), 8.53-8.45 (m, 2H), 8.32-8.24 (m, 1H), 8.08 (d, J=8.2 Hz, 1H), 8.03 (s, 1H), 7.92 (s, 4H), 7.86 (d, J=8.1 Hz, 1H), 7.75-7.67 (m, 1H), 7.56 (d, J=7.6 Hz, 1H), 7.44 (d, J=8.0 Hz, 1H), 7.39 (d, J=7.4 Hz, 1H), 7.34 (d, J=8.3 Hz, 2H), 7.26 (t, J=10.3 Hz, 2H), 4.90 (s, 1H), 4.83-4.64 (m, 2H), 4.60 (d, J=9.3 Hz, 1H), 4.53 (t, J=8.2 Hz, 1H), 4.36 (s, 1H), 4.31 (d, J=5.5 Hz, 1H), 4.29-4.21 (m, 3H), 3.85-3.79 (m, 2H), 3.42-3.34 (m, 2H), 2.67 (s, 3H), 2.19-1.75 (m, 7H), 1.58-1.46 (m, 2H), 1.41-1.19 (m, 8H), 0.96 (s, 9H).

### Example A376. 2-amino-3-ethynyl-N-((S)-6-(6-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) bicyclo[2.2.2]octan-1-yl)carbamoyl)pyridin-3-yl)-2,3-dihydrobenzofuran-3-yl)-N-methylquinoline-6-carboxamide

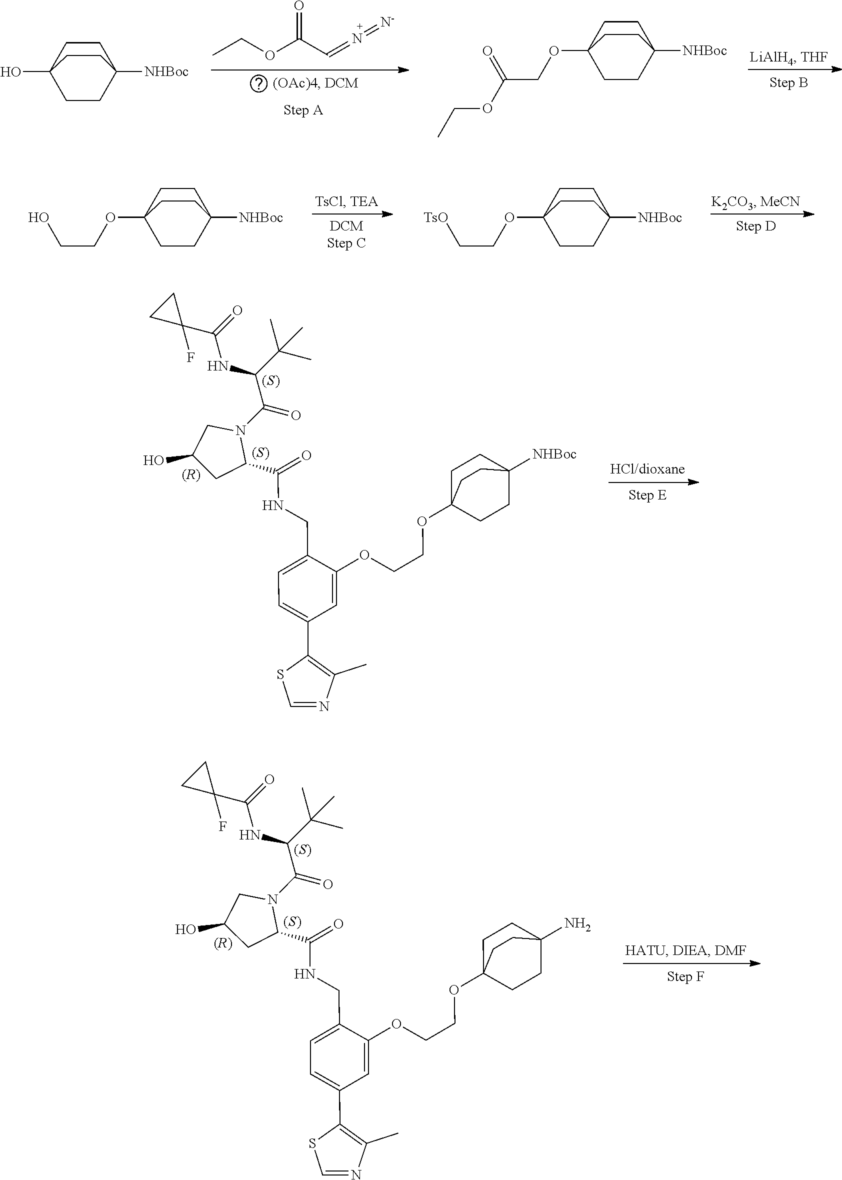

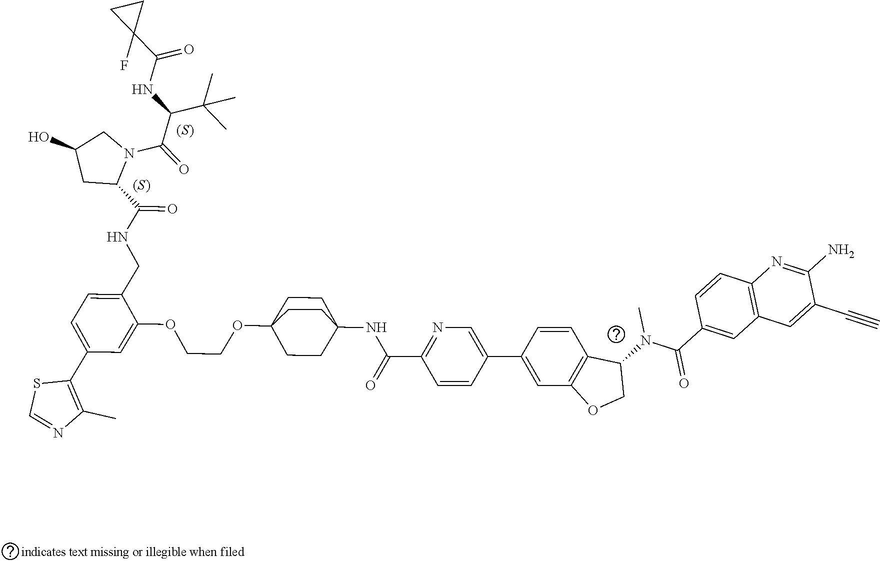

Example A376 was synthesized following the same procedures as described in Example A370 (9.7 mg, 1.6% yield over 6 steps) as a white solid. LC-MS (ESI): mass calcd. for C₆₃H₆₈FN₉O₉S, 1145.48 m/z found, 1146.1 [M+H]⁺. ¹H NMR (400 MHz, DMSO-d₆) δ 8.99 (s, 1H), 8.92 (s, 1H), 8.64 (s, 1H), 8.48 (t, J=5.7 Hz, 1H), 8.28 (d, J=8.1 Hz, 1H), 8.05 (d, J=8.1 Hz, 2H), 7.95 (s, 1H), 7.87 (d, J=8.2 Hz, 1H), 7.73 (s, 1H), 7.55 (d, J=7.8 Hz, 1H), 7.40 (d, J=7.7 Hz, 2H), 7.34 (s, 1H), 7.29 (d, J=6.6 Hz, 1H), 7.03 (s, 1H), 6.96 (d, J=7.9 Hz, 1H), 4.92 (s, 1H), 4.73-4.66 (m, 2H), 4.60 (d, J=9.4 Hz, 1H), 4.52 (t, J=8.3 Hz, 2H), 4.36 (s, 1H), 4.34-4.26 (m, 1H), 4.25-4.18 (m, 1H), 4.11 (s, 2H), 3.74-3.52 (m, 5H), 2.66 (s, 3H), 2.46 (s, 3H), 2.14-2.06 (m, 7H), 1.97-1.91 (m, 1H), 1.78 (s, 6H), 1.40-1.31 (m, 2H), 1.24-1.20 (m, 2H), 0.96 (s, 9H).

### Example A378. 2-amino-3-cyano-N-((S)-6-(6-(4-((4-fluoro-1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N-methylquinoline-6-carboxamide

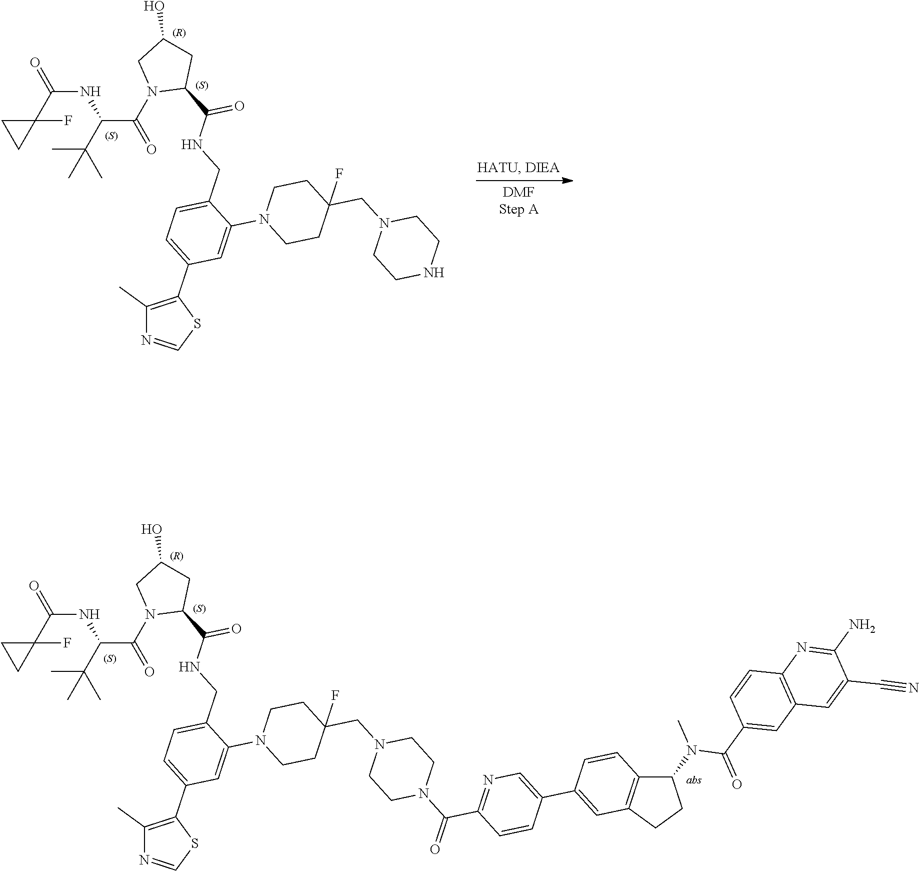

Example A378 was synthesized following the same procedures as described in Example A23 (3.7 mg, 9% yield) as a white solid. LC-MS (ESI): mass calcd, for C₆₂H₆₈F₂N₁₂O₇S, 1162.50; m/z found, 1163.50 [M+H]⁺. ¹H NMR (400 MHz, DMSO-d₆) δ 9.00 (s, 1H), 8.94 (s, 1H), 8.83 (s, 1H), 8.53 (t, J=5.5 Hz, 1H), 8.28 (dd, J=8.2, 2.1 Hz, 1H), 7.98 (s, 1H), 7.82 (dd, J=8.8, 1.8 Hz, 1H), 7.77 (d, J=8.2 Hz, 1H), 7.58 (dd, J=13.7, 8.0 Hz, 2H), 7.46 (d, J=7.8 Hz, 2H), 7.42-7.33 (m, 3H), 7.27 (dd, J=9.3, 2.3 Hz, 1H), 7.19-7.14 (m, 2H), 4.81-4.67 (m, 3H), 4.61-4.59 (m, 1H), 4.51-4.49 (m, 1H), 4.44-4.39 (m, 4H), 3.68-3.59 (m, 4H), 3.39-3.19 (m, 3H), 3.05-2.92 (m, 4H), 2.68 (s, 3H), 2.54-2.51 (m, 4H), 2.46 (s, 3H), 2.12-2.04 (m, 4H), 2.02-1.87 (m, 2H), 1.41-1.32 (m, 2H), 1.22 (d, J=8.5 Hz, 2H), 0.97 (s, 9H).

### Example A379. N1-(6-amino-5-cyanopyridin-3-yl)-N2-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N2-methyloxalamide

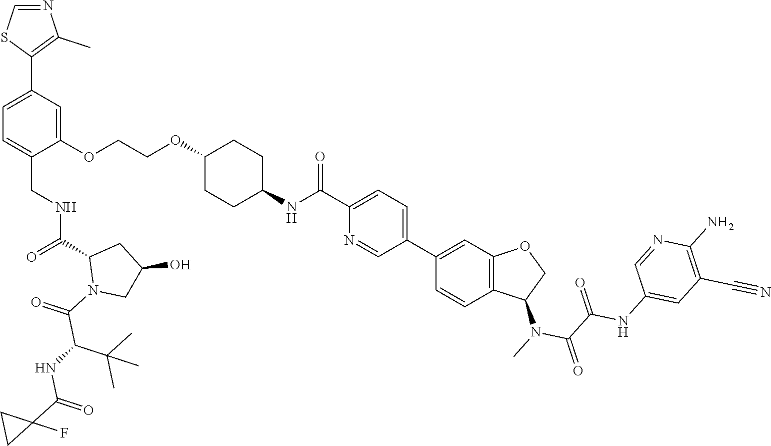 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)C(=O)Nc4cnc(N)c(C#N)c4)cn3)CC2)c1]

Example A379 was synthesized following the same procedures as described in Example A102 as a white solid. LC-MS (ESI): mass calcd, for C₅₇H₆₄FN₁₁O₁₀S, 1113.45 m/z found, 1114 [M+H]⁺. ¹HNMR (400 MHz, DMSO-d₆) δ 10.88 (d, J=50.9 Hz, 1H), 8.99 (s, 1H), 8.93 (s, 1H), 8.55-8.45 (m, 2H), 8.42 (dd, J=15.2, 2.6 Hz, 1H), 8.28 (dd, J=8.2, 1.8 Hz, 1H), 8.17-7.99 (m, 2H), 7.58-7.33 (m, 4H), 7.31-7.26 (m, 1H), 7.24-7.00 (m, 2H), 6.99-6.87 (m, 2H), 6.29-5.94 (m, 1H), 4.81-4.70 (m, 1H), 4.68-4.57 (m, 2H), 4.52 (t, J=8.3 Hz, 1H), 4.38-4.23 (m, 3H), 4.20-4.16 (m, 2H), 3.84-3.77 (m, 5H), 3.42-3.33 (m, 3H), 2.77-2.61 (m, 3H), 2.47 (s, 3H), 2.07-2.03 (m, 2H), 2.00 (d, J=7.8 Hz, 1H), 1.96-1.91 (m, 1H), 1.88-1.82 (m, 2H), 1.55-1.47 (m, 2H), 1.39-1.33 (m, 2H), 1.24-1.22 (m, 4H), 0.96 (s, 9H).

### Example A380. 2-amino-3-cyclopropyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(prop-1-yn-1-yl)phenoxy)ethoxy)cyclohexyl)carbamoyl)pyridin-3-yl)-2,3-dihydrobenzofuran-3-yl)-N-methylquinoline-6-carboxamide

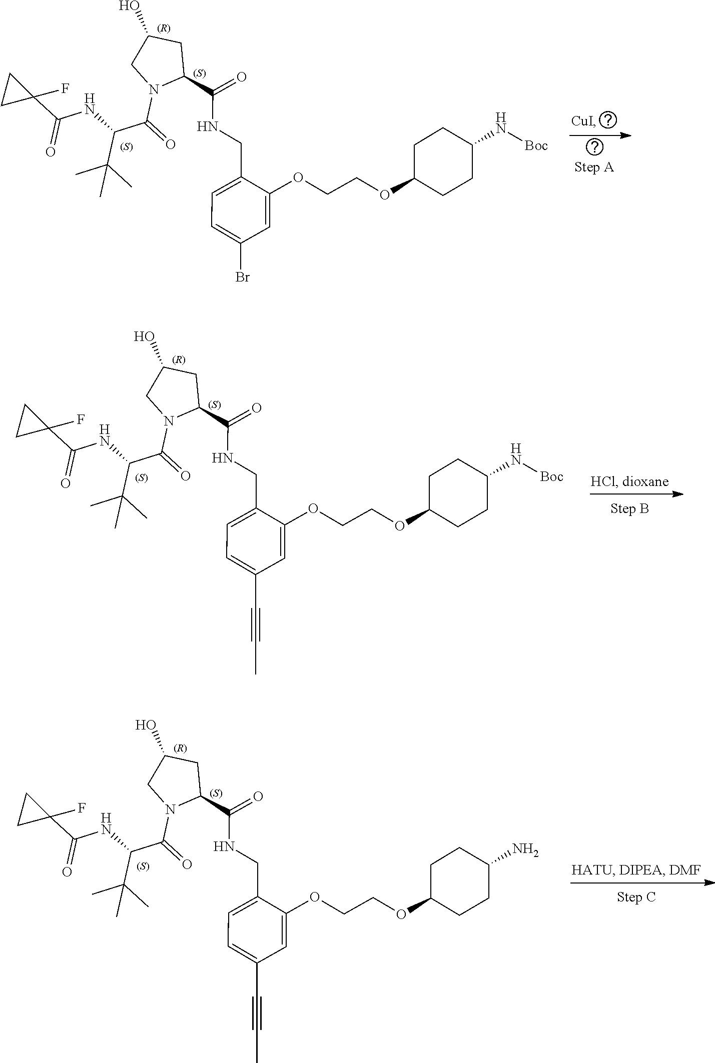

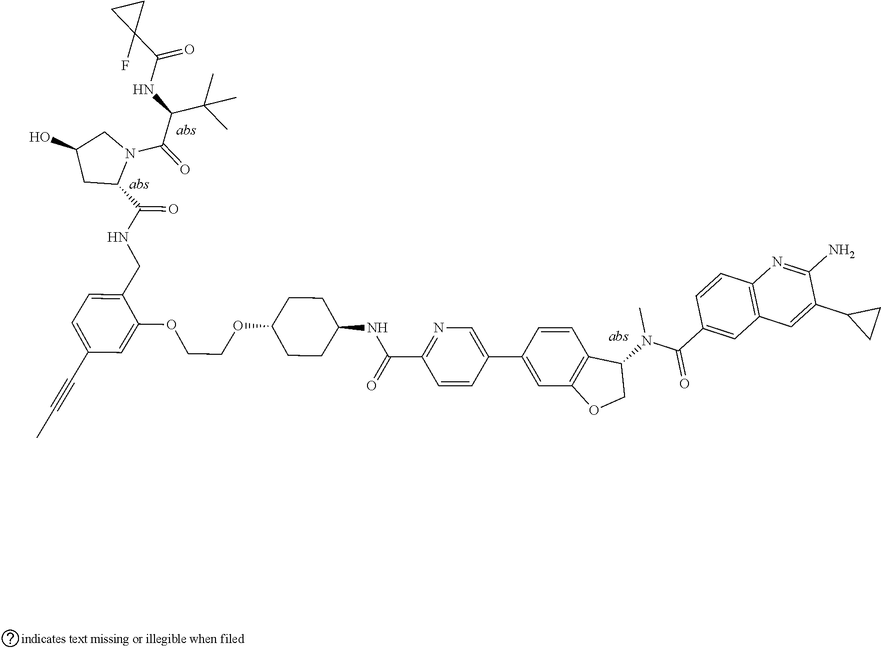

Example A380 was synthesized following the same procedures as described in Example A39 (3.9 mg, 5% yield over 3 steps) as a white solid. LC-MS (ESI): mass calcd, for C₆₁H₆₉FN₈O₉, 1076.5/z found, 1078.1 [M+H]⁺. ¹HNMR (400 MHz, DMSO-d₆) δ 8.93 (s, 1H), 8.83 (s, 2H), 8.58-8.40 (m, 2H), 8.33-8.18 (m, 2H), 8.15-7.97 (m, 2H), 7.86 (d, J=8.3 Hz, 1H), 7.81-7.68 (m, 1H), 7.55 (d, J=7.0 Hz, 1H), 7.46-7.33 (m, 2H), 7.31-7.19 (m, 2H), 7.18-6.74 (m, 2H), 4.86-4.65 (m, 2H), 4.59 (d, J=9.0 Hz, 1H), 4.49 (t, J=8.2 Hz, 1H), 4.35 (s, 1H), 4.28-4.06 (m, 5H), 3.78 (s, 4H), 3.42-3.29 (m, 2H), 2.66 (s, 3H), 2.09-2.01 (m, 5H), 1.99-1.80 (m, 4H), 1.56-1.46 (m, 2H), 1.41-1.28 (m, 4H), 1.27-1.18 (m, 3H), 1.14-1.06 (m, 2H), 0.95 (s, 9H), 0.86-0.74 (m, 2H).

Example A383. 2-amino-3-ethynyl-N-((S)-6-(6-(4-(3-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy) propyl)piperazine-1-carbonyl)pyridin-3-yl)-2,3-dihydrobenzofuran-3-yl)-N-methylquinoline-6-carboxamide

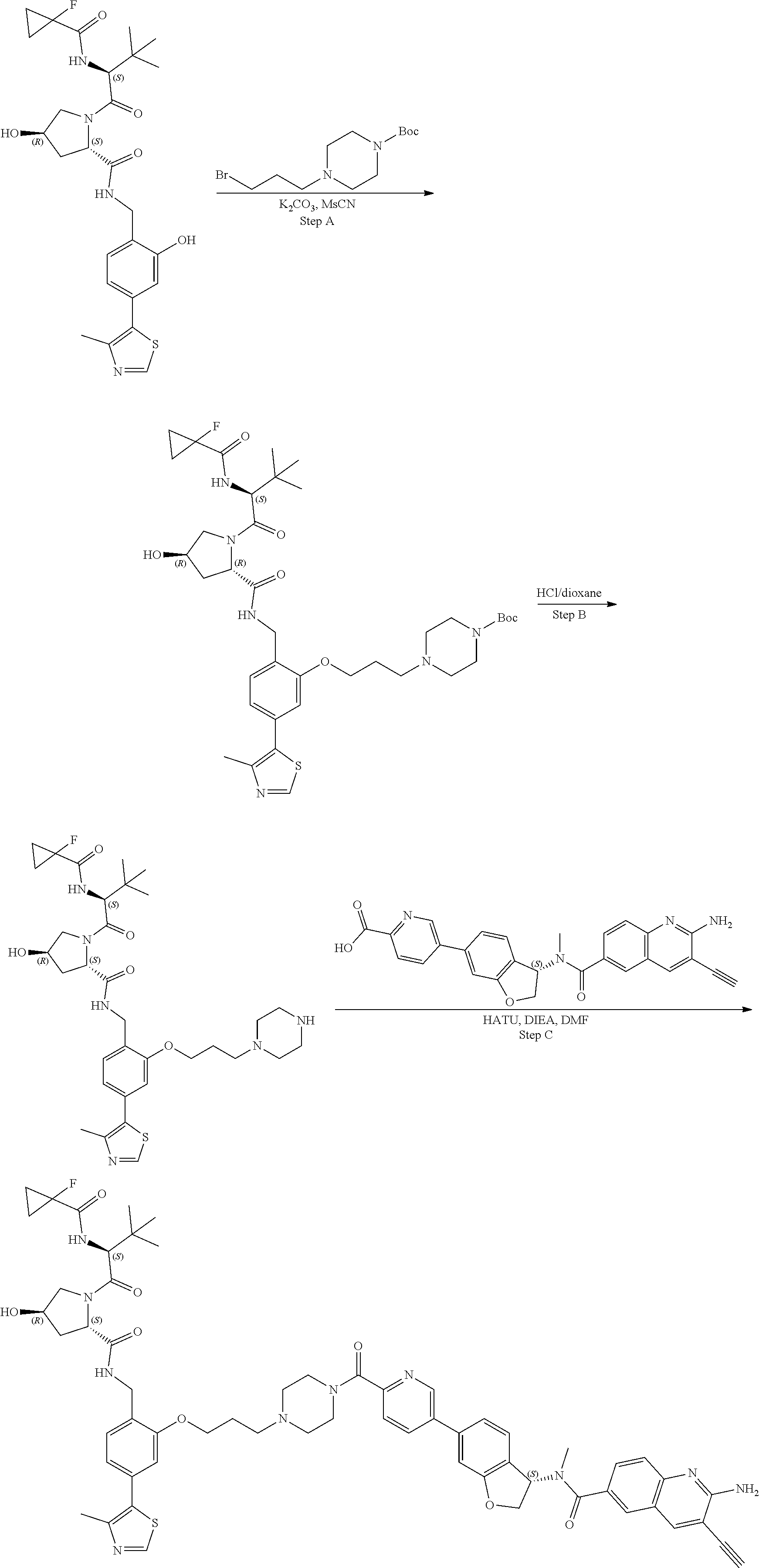

Example A383 was synthesized following the same procedures as described in Example A29 (7 mg, 8% yield over 3 steps) as a white solid. LC-MS (ESI): mass calcd, for C₆₀H₆₅FN₁₀O₈S, 1104.5/z found, 1106.0 [M+H]⁺. ¹H NMR (400 MHz, DMSO-d₆) δ 9.00 (s, 1H), 8.93 (s, 1H), 8.61 (s, 1H), 8.56-8.50 (m, 1H), 8.37-8.18 (m, 2H), 8.04 (s, 1H), 7.86 (d, J=7.8 Hz, 1H), 7.78 (d, J=8.1 Hz, 1H), 7.71 (s, 1H), 7.56 (d, J=7.7 Hz, 1H), 7.46-7.38 (m, 2H), 7.35 (s, 1H), 7.27 (d, J=6.7 Hz, 1H), 7.04-6.96 (m, 2H), 4.91 (s, 1H), 4.72-4.67 (m, 2H), 4.60-4.58 (m, 1H), 4.54-4.51 (m, 1H), 4.37-4.33 (m, 2H), 4.31-4.28 (m, 3H), 4.17-4.14 (m, 2H), 3.74-3.69 (m, 1H), 3.67-3.61 (m, 2H), 3.60-3.45 (m, 3H), 3.43-3.36 (m, 2H), 3.28-3.15 (m, 3H), 2.67 (s, 3H), 2.47 (s, 3H), 2.23-2.14 (m, 2H), 2.12-2.05 (m, 1H), 1.95-1.86 (m, 1H), 1.41-1.30 (m, 2H), 1.25-1.19 (m, 2H), 0.94 (s, 9H).

### Example A384. 2-amino-3-ethynyl-N-((S)-6-(6-(((1R,4S)-4-(2-(2-((R)-1-((28,4R)-1-((S)-2-(1-fluorocyclopropane-1-carboxamido)-3,3-dimethylbutanoyl)-4-hydroxypyrrolidine-2-carboxamido)-2-morpholinocthyl)-5-(4-methylthiazol-5-yl)phenoxy)ethoxy)cyclohexyl) carbamoyl)pyridin-3-yl)-2,3-dihydrobenzofuran-3-yl)-N-methylquinoline-6-carboxamide

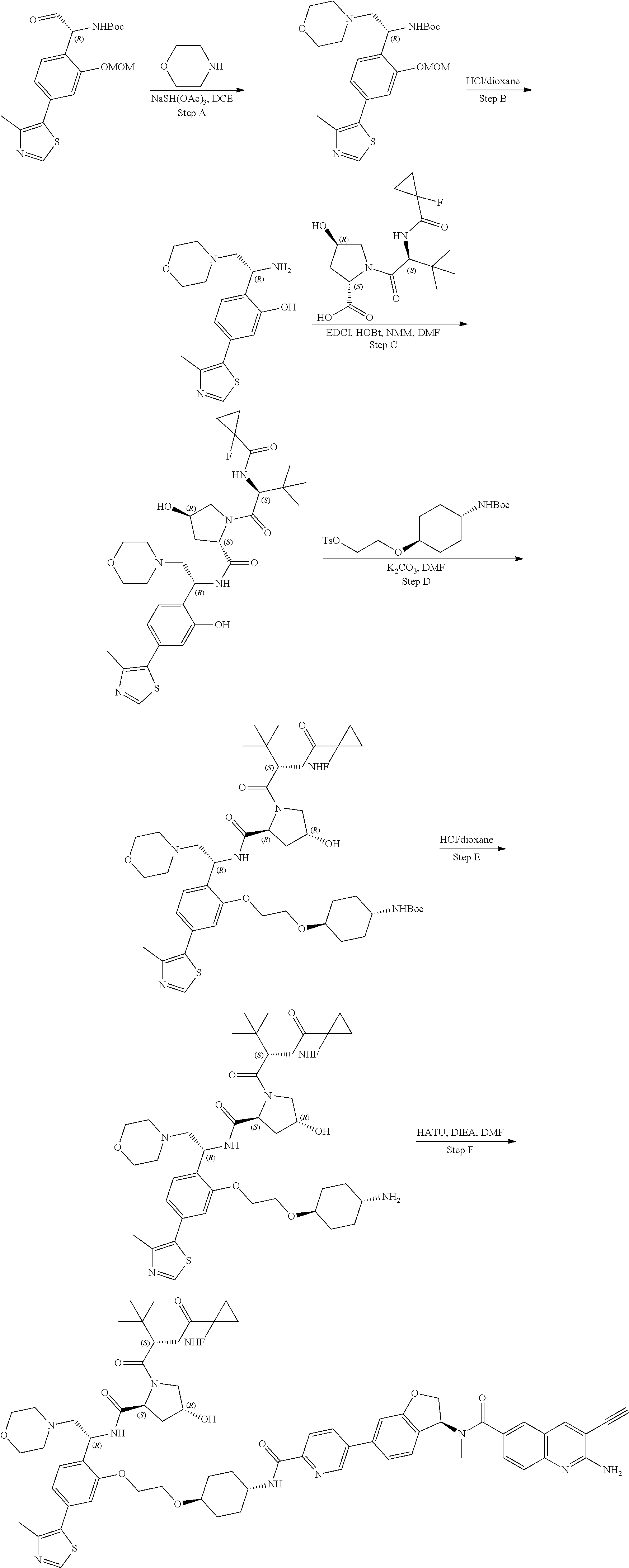

Step A: tert-butyl (R)-(1-(2-(methoxymethoxy)-4-(4-methylthiazol-5-yl)phenyl)-2-morpholinoethyl)carbamate

To a mixture of tert-butyl (R)-(1-(2-(methoxymethoxy)-4-(4-methylthiazol-5-yl)phenyl)-2-oxoethyl)carbamate (200 mg, 510 μmol, 1.0 eq.) in DMF (6 mL) was added morpholine (89 mg, 1.02 mmol, 2.0 eq.) and Sodium cyanoborohydride (96 mg, 1.53 mmol, 3.0 eq.). The reaction mixture was stirred at room temperature for 16 hours. The mixture was quenched water (30 mL), extracted with EtOAc (30 mL×3) and the layers were separated. The organic layer was washed with brine (30 mL), dried over Na₂SO₄, filtered and concentrated. The product was purified by column chromatography (silica gel, 0% EtOAc in PE~50% EtOAc in PE) to afford tert-butyl (R)-(1-(2-(methoxymethoxy)-4-(4-methylthiazol-5-yl)phenyl)-2-morpholinoethyl)carbamate (80 mg, 34% yield) as a yellow oil. LC-MS (ESI): mass calcd. For C₂₃H₃₃N305S, 463.21; m/z found, 463.8 [M+H]⁺

Step B: (R)-2-(1-amino-2-morpholinoethyl)-5-(4-methylthiazol-5-yl) phenol

To a mixture of tert-butyl (R)-(1-(2-(methoxymethoxy)-4-(4-methylthiazol-5-yl)phenyl)-2-morpholinoethyl)carbamate (80 mg, 173 μmol, 1.0 eq.) in DCM (2 mL) was added HCl/1,4-dioxane (1 mL, 4 M/L). The reaction mixture was stirred at room temperature for 1 hour. The mixture was concentrated to afford (R)-2-(1-amino-2-morpholinoethyl)-5-(4-methylthiazol-5-yl) phenol (55.0 mg, 99% yield) as a yellow solid. LC-MS (ESI): mass calcd. For C₁₆H₂₁N₃O₂S, 319.13; m/z found, 319.7 [M+H]⁺.

Steps C-F: 2-amino-3-ethynyl-N-(S)-6-(6-(((1R,4S)-4-(2-(2-((R)-1-(28,4R)-1-(S)-2-(1-fluorocyclopropane-1-carboxamido)-3,3-dimethylbutanoyl)-4-hydroxypyrrolidine-2-carboxamido)-2-morpholinoethyl)-5-(4-methylthiazol-5-yl)phenoxy)ethoxy)cyclohexyl) carbamoyl)pyridin-3-yl)-2,3-dihydrobenzofuran-3-yl)-N-methylquinoline-6-carboxamide

Example A384 was synthesized following the same procedures as described in Example A363 (2.2 mg, 2% yield over 4 steps) as a white solid. LC-MS (ESI): mass calcd. for C₆₆H₇₅FN₁₀O₁₀S, 1218.54; m/z found, 1219.3 [M+H]⁺. ¹H NMR (400 MHz, DMSO-d₆) δ 8.99 (s, 1H), 8.92 (s, 1H), 8.56-8.46 (m, 1H), 8.28 (dd, J=11.0, 2.5 Hz, 2H), 8.08 (d, J=8.1 Hz, 1H), 7.87 (s, 1H), 7.66 (d, J=8.6 Hz, 1H), 7.54 (t, J=9.4 Hz, 2H), 7.38 (d, J=7.6 Hz, 1H), 7.35-7.25 (m, 2H), 7.01 (d, J=8.1 Hz, 2H), 6.74 (s, 2H), 5.33 (s, 1H), 5.19 (s, 1H), 4.85-4.73 (m, 1H), 4.68 (d, J=9.9 Hz, 1H), 4.63 (s, 1H), 4.61-4.52 (m, 2H), 4.35 (d, J=24.7 Hz, 1H), 4.19 (s, 2H), 4.00 (s, 1H), 3.82 (s, 4H), 3.70-3.58 (m, 4H), 3.58-3.49 (m, 4H), 3.38 (s, 1H), 3.31-3.31 (m, 2H), 2.67 (s, 3H), 2.47 (d, J=3.1 Hz, 3H), 2.16-1.99 (m, 4H), 1.85 (dd, J=7.6, 5.4 Hz, 2H), 1.56-1.46 (m, 2H), 1.40-1.28 (m, 4H), 1.21 (s, 2H), 0.93 (d, J=27.4 Hz, 9H).

### Example A385. N1-(6-amino-5-cyclopropylpyridin-3-yl)-N2-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-N2-methyloxalamide

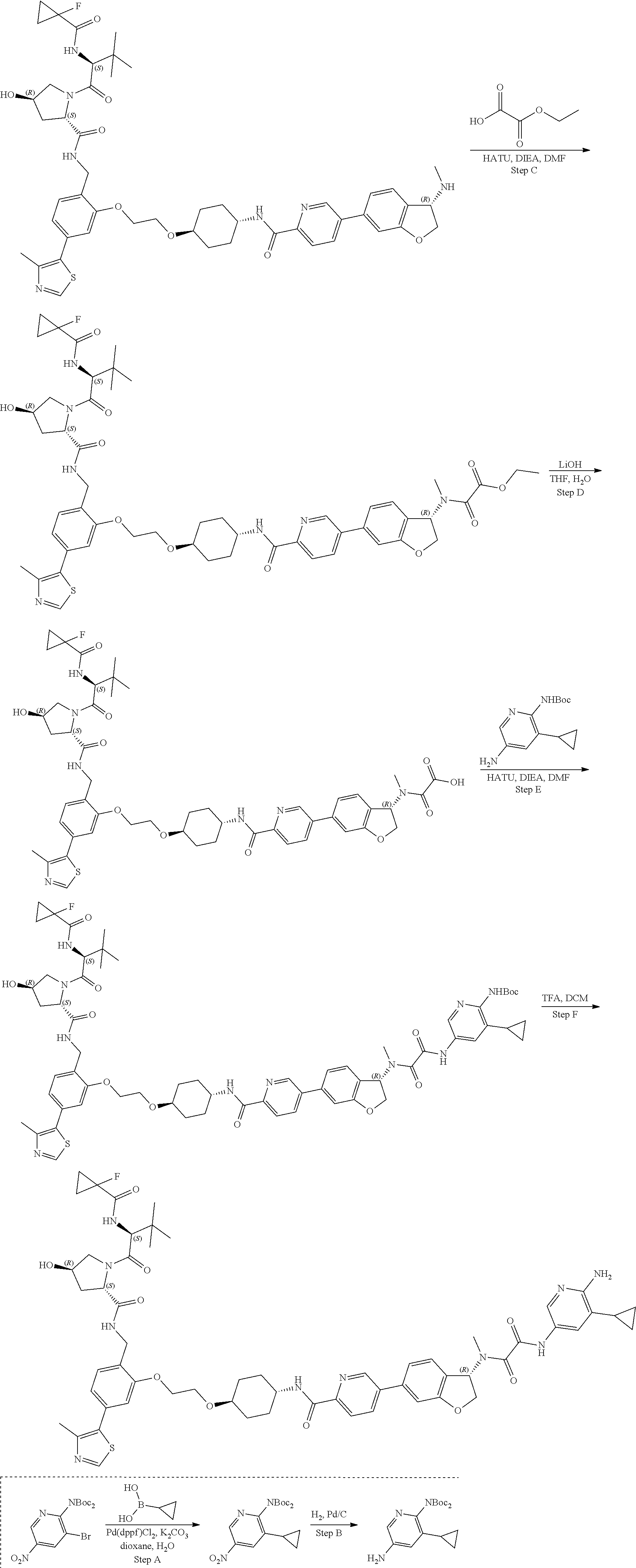

Step A: tert-butyl (tert-butoxycarbonyl)(3-cyclopropyl-5-nitropyridin-2-yl)carbamate

To a mixture of tert-butyl (3-bromo-5-nitropyridin-2-yl)(tert-butoxycarbonyl)carbamate (500 mg, 1.20 mmol, 1.0 eq.) in 1,4-Dioxane (10 mL) and Water (1 mL) was added cyclopropylboronic acid (308 mg, 3.59 mmol, 3.0 eq.), PdCl₂ (dppf)(88 mg, 120 μmol, 0.1 eq.) and K₂CO₃ (496 mg, 3.59 mmol, 3.0 eq.). The reaction mixture was stirred at 100° C. for 4 hours. The mixture was quenched water (50 mL), extracted with EtOAc (50 mL×3) and the layers were separated. The organic layer was washed with brine, dried over Na₂SO₄, filtered and concentrated. The product was purified by column chromatography (silica gel, 0% EtOAc in PE~10% EtOAc in PE) to afford tert-butyl (tert-butoxycarbonyl)(3-cyclopropyl-5-nitropyridin-2-yl)carbamate (250 mg, 55% yield) as a yellow oil. LC-MS (ESI): mass calcd. For C₁₈H₂₅N₃O₆, 379.17; m/z found, 379.7 [M+H]⁺.

Step B: tert-butyl (5-amino-3-cyclopropylpyridin-2-yl)(tert-butoxycarbonyl)carbamate

To a mixture of tert-butyl (tert-butoxycarbonyl)(3-cyclopropyl-5-nitropyridin-2-yl)carbamate (250 mg, 659 μmol, 1.0 eq.) in MeOH (10 mL) was added Pd/C (25 mg, 235 μmol, 0.4 eq.). The reaction mixture was stirred at 30° C. for 12 hours with H₂. The mixture was filtered and concentrated to afford tert-butyl (5-amino-3-cyclopropylpyridin-2-yl)(tert-butoxycarbonyl)carbamate (200 mg, 87% yield) as a yellow solid. LC-MS (ESI): mass calcd. For C₁₈H₂₇N₃O₄, 349.20; m/z found, 349.7 [M+H]⁺.

Steps C-F: N1-(6-amino-5-cyclopropylpyridin-3-yl)-N2-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N2-methyloxalamide

Example A385 was synthesized following the same procedures as described in Example A44 (2.1 mg, 2% yield over 4 steps) as a white solid. LC-MS (ESI): mass calcd. for C₅₉H₆₉FN₁₀O₁₀S, 1128.49; m/z found, 1127.4 [M+H]⁺. ¹H NMR (400 MHz, DMSO-d₆) δ 11.06 (d, J=34.6 Hz, 1H), 8.99 (s, 1H), 8.92 (d, J=1.9 Hz, 1H), 8.53-8.45 (m, 2H), 8.36 (dd, J=13.0, 2.2 Hz, 1H), 8.27 (dt, J=8.2, 2.0 Hz, 1H), 8.08 (d, J=8.2 Hz, 1H), 8.01 (s, 1H), 7.74 (dd, J=21.4, 1.8 Hz, 1H), 7.50 (dd, J=43.9, 7.9 Hz, 1H), 7.40 (t, J=7.3 Hz, 2H), 7.37 (s, 1H), 7.29 (dd, J=9.2, 2.6 Hz, 1H), 7.05 (d, J=1.3 Hz, 1H), 6.97 (dd, J=7.9, 1.2 Hz, 1H), 6.27-5.97 (m, 1H), 4.81-4.72 (m, 1H), 4.66 (dd, J=11.0, 4.0 Hz, 1H), 4.59 (d, J=8.9 Hz, 1H), 4.51 (d, J=8.2 Hz, 1H), 4.35 (d, J=4.1 Hz, 1H), 4.30 (d, J=6.0 Hz, 1H), 4.25 (d, J=5.7 Hz, 1H), 4.17 (d, J=4.5 Hz, 2H), 3.82-3.80 (m, 4H), 3.66 (d, J=7.1 Hz, 1H), 3.60 (d, J=10.9 Hz, 1H), 3.39-3.33 (m, 1H), 2.70 (d, J=61.5 Hz, 3H), 2.47 (s, 3H), 2.11 (d, J=13.2 Hz, 1H), 2.05 (d, J=16.7 Hz, 2H), 1.97-1.90 (m, 1H), 1.86 (s, 1H), 1.86-1.80 (m, 2H), 1.51 (dd, J=23.7, 10.5 Hz, 2H), 1.34 (ddd, J=18.3, 11.3, 4.1 Hz, 4H), 1.21 (dd, J=8.6, 3.3 Hz, 2H), 1.07-1.02 (m, 2H), 0.95 (s, 9H), 0.68-0.63 (m, 2H).

### Example A386. 2-amino-3-ethynyl-N-((S)-6-(6-(((1S,4S)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenethoxy)cyclohexyl)carbamoyl)pyridin-3-yl)-2,3-dihydrobenzofuran-3-yl)-N-methylquinoline-6-carboxamide

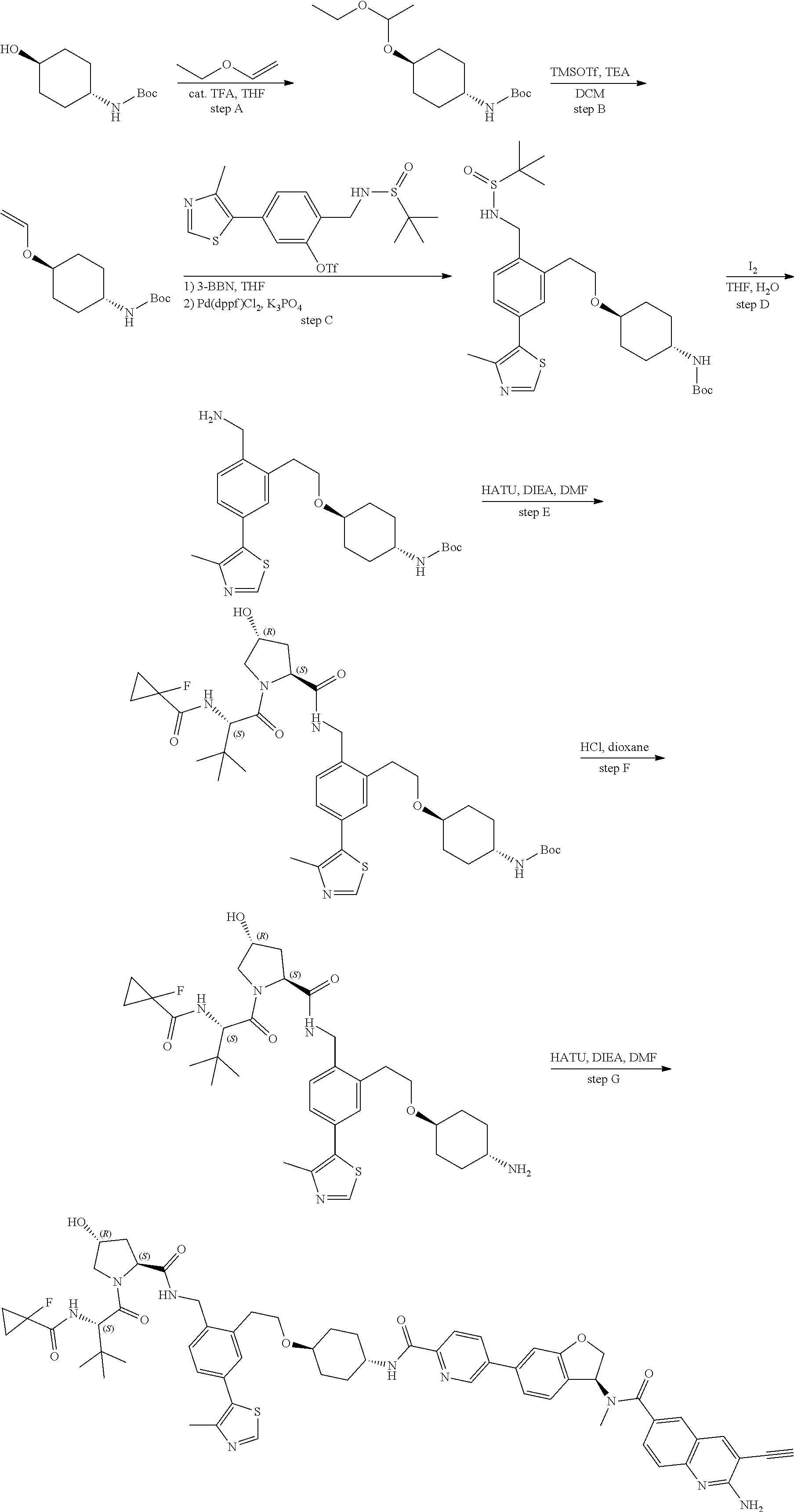

Step A: tert-butyl ((1r,4r)-4-(1-ethoxyethoxy)cyclohexyl)carbamate

To a stirred mixture of tert-butyl ((1r,4r)-4-hydroxycyclohexyl)carbamate (2 g, 9.30 mmol, 1.0 eq.) in ethoxyethene (20 mL) was added TFA (0.1 mL, 0.93 mmol, 0.1 eq.) slowly at 0° C. The resulting mixture was stirred at room temperature for 3 hours. The reaction mixture was quenched with NaHCO₃(aq. 50 mL), and then extracted with EtOAc. The combined organic layers were washed with brine, dried over anhydrous Na₂SO₄ and concentrated. The residue was purified by column chromatography (silica gel, 0% EtOAc in PE~10% EtOAc in PE) to afford tert-butyl ((1r,4r)-4-(1-ethoxyethoxy)cyclohexyl)carbamate (800 mg, 30% yield) as a white solid. ¹HNMR (400 MHz, CDCl₃) δ 4.77 (q, J=5.3 Hz, 1H), 4.43-4.30 (m, 1H), 3.66-3.59 (m, 1H), 3.52-3.38 (m, 3H), 2.04-1.93 (m, 4H), 1.44 (s, 9H), 1.41-1.24 (m, 5H), 1.20 (t, J=7.1 Hz, 3H), 1.17-1.08 (m, 2H).

Step B: tert-butyl ((1r,4r)-4-(1-ethoxyethoxy)cyclohexyl)carbamate

To a stirred mixture of tert-butyl ((1r,4r)-4-(1-ethoxyethoxy)cyclohexyl)carbamate (400 mg, 1.39 mmol, 1.0 eq.) in DCM (20 mL) was added TEA (0.5 mL, 3.48 mmol, 2.5 eq.), followed by TMSOTf (0.5 mL, 2.79 mmol, 2.0 eq.) slowly at 0° C. The resulting mixture was stirred at room temperature for 3 hours. The reaction mixture was quenched with water (30 mL), and then extracted with DCM. The combined organic layers were washed with brine, dried over anhydrous Na₂SO₄ and concentrated. The residue was purified by column chromatography (silica gel, 0% EtOAc in PE~10% EtOAc in PE) to afford tert-butyl ((1r,4r)-4-(vinyloxy)cyclohexyl)carbamate (120 mg, 35% yield) as a white solid. ¹H NMR (400 MHz, CDCl₃) δ 6.30 (dd, J=14.1, 6.6 Hz, 1H), 4.38 (s, 1H), 4.28 (dd, J=14.1, 1.5 Hz, 1H), 4.00 (dd, J=6.6, 1.5 Hz, 1H), 3.74-3.65 (m, 1H), 3.46 (s, 1H), 2.07-2.00 (m, 4H), 1.47-1.40 (m, 11H), 1.26-1.16 (m, 2H).

Step C: tert-butyl ((1r, 4r)-4-(2-(((tert-butylsulfinyl)amino)methyl)-5-(4-methylthiazol-5-yl) phenethoxy)cyclohexyl)carbamate

To a solution of tert-butyl ((1r,4r)-4-(vinyloxy)cyclohexyl)carbamate (150 mg, 622 μmol, 1.9 eq.) in THF (5 mL) were added 9-BBN (120 mg, 493 μmol, 1.5 eq.) at 0° C. The mixture was stirred at 0° C. for 2 hours. 2-(((tert-butylsulfinyl)amino)methyl)-5-(4-methylthiazol-5-yl) phenyl trifluoromethanesulfonate (150 mg, 329 μmol, 1.0 eq.) in DMF (2 mL) and K₂CO₃ (136 mg, 986 μmol, 3.0 eq.) was added, followed by 1,1′-Bis(di-t-butylphosphino) ferrocene palladium dichloride (21 mg, 32.9 μmol, 0.1 eq.). The mixture was stirred at 100° C. overnight. The reaction mixture was poured into water (20 mL) and then extracted with DCM (30 mL×2). The combined organic layers were washed with brine (50 mL), dried over anhydrous sodium sulfate, concentrated under reduced pressure, and purified by silica gel chromatography (silica gel, 0% EtOAc in PE~50% EtOAc in PE) to afford tert-butyl ((1r,4r)-4-(2-(((tert-butylsulfinyl)amino)methyl)-5-(4-methylthiazol-5-yl) phenethoxy)cyclohexyl)carbamate (35 mg, 19% yield) as a yellow solid. LC-MS (ESI): mass calcd. For C₂₈H₄₃N304S2, 549.27; m/z found, 550.5 [M+H]⁺.

Steps D G: 2-amino-3-ethynyl-N-((S)-6-(6-(((1S,4S)-4-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenethoxy)cyclohexyl)carbamoyl)pyridin-3-yl)-2,3-dihydrobenzofuran-3-yl)-N-methylquinoline-6-carboxamide

Example A386 was synthesized following the same procedures as described in Example A13 (6.5 mg, 1% yield over 4 steps) as a white solid. LC-MS (ESI): mass calcd, for C₆₁H₆₆FN₉O₈S, 1103.5/z found, 1104.9 [M+H]⁺. ¹HNMR (400 MHz, DMSO-d₆) δ 8.93 (s, 1H), 8.83 (s, 2H), 8.58-8.40 (m, 2H), 8.33-8.18 (m, 2H), 8.15-7.97 (m, 2H), 7.86 (d, J=8.3 Hz, 1H), 7.81-7.68 (m, 1H), 7.55 (d, J=7.0 Hz, 1H), 7.46-7.33 (m, 2H), 7.31-7.19 (m, 2H), 7.18-6.74 (m, 2H), 4.86-4.65 (m, 2H), 4.59 (d, J=9.0 Hz, 1H), 4.49 (t, J=8.2 Hz, 1H), 4.35 (s, 1H), 4.28-4.06 (m, 5H), 3.78 (s, 4H), 3.42-3.29 (m, 2H), 2.66 (s, 3H), 2.09-2.01 (m, 5H), 1.99-1.80 (m, 4H), 1.56-1.46 (m, 2H), 1.41-1.28 (m, 4H), 1.27-1.18 (m, 3H), 1.14-1.06 (m, 2H), 0.95 (s, 9H), 0.86-0.74 (m, 2H).

### Example A387. N1-(6-amino-5-ethynylpyridin-3-yl)-N2-((S)-6-(6-(((18,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) cyclohexyl)carbamoyl)pyridin-3-yl)-2,3-dihydrobenzofuran-3-yl)-N2-methyloxalamide

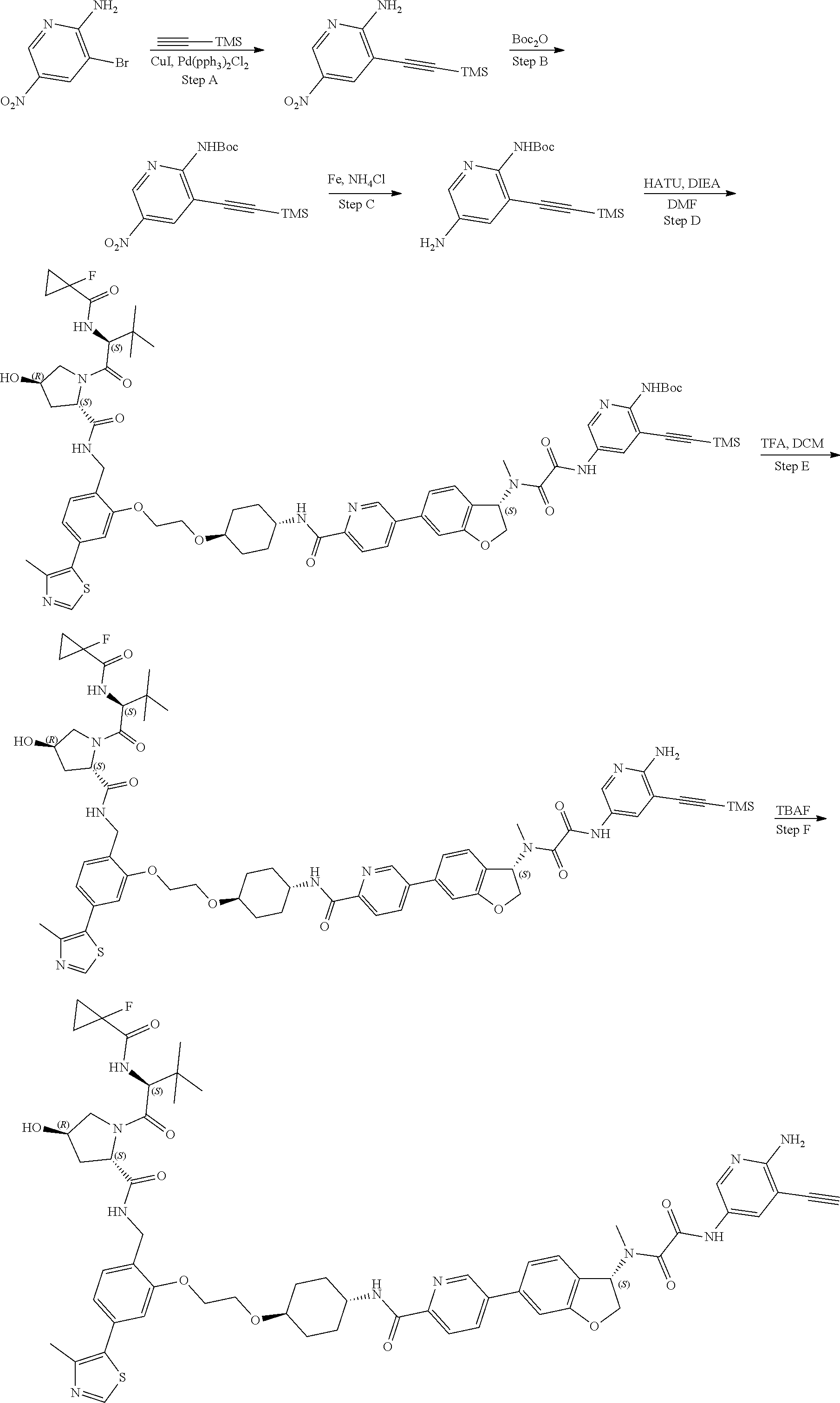

Example A387 was synthesized following the same procedures as described in Example A338 (2.0 mg, 7% yield over 2 steps) as a white solid. LC-MS (ESI): mass calcd. for C₅₈H₆₅FN₁₀O₁₀S, 1112.46; m/z found, 1113.5 [M+H]⁺. ¹H NMR (400 MHz, MeOD) δ 8.92 (s, 1H), 8.87 (s, 1H), 8.50 (dd, J=17.7, 2.4 Hz, 1H), 8.18 (d, J=2.4 Hz, 1H), 8.15 (d, J=8.1 Hz, 1H), 7.58-7.52 (m, 1H), 7.50 (d, J=7.0 Hz, 1H), 7.46 (d, J=5.8 Hz, 1H), 7.34 (d, J=7.8 Hz, 1H), 7.25 (d, J=4.9 Hz, 1H), 7.07 (d, J=1.4 Hz, 1H), 7.03 (dd, J=7.8, 1.5 Hz, 1H), 6.35 (dd, J=8.6, 3.6 Hz, 1H), 6.18 (dd, J=8.7, 3.6 Hz, 1H), 4.92-4.86 (m, 2H), 4.81 (d, J=8.0 Hz, 4H), 4.78 (d, J=3.3 Hz, 1H), 4.75 (d, J=8.9 Hz, 2H), 4.68-4.61 (m, 2H), 4.53-4.47 (m, 2H), 4.43 (d, J=15.8 Hz, 1H), 4.30 (d, J=2.4 Hz, 1H), 4.25-4.21 (m, 2H), 3.95-3.89 (m, 3H), 3.87-3.76 (m, 2H), 3.53-3.47 (m, 1H), 3.24 (dd, J=10.1, 7.0 Hz, 3H), 2.91 (s, 1H), 2.74 (s, 2H), 2.50 (s, 3H), 2.28-2.21 (m, 1H), 2.19-2.15 (m, 2H), 2.07-2.03 (m, 2H), 1.68-1.63 (m, 2H), 1.52-1.48 (m, 2H), 1.44-1.39 (m, 3H), 1.33-1.31 (m, 2H), 1.03 (s, 9H).

### Example A389. 2-amino-3-cyano-N-((S)-6-(6-((4-(2-(2-(((2S,4R)-1-((S)-2-cyclohexyl-2-(1-fluorocyclopropane-1-carboxamido) acetyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methylquinoline-6-carboxamide

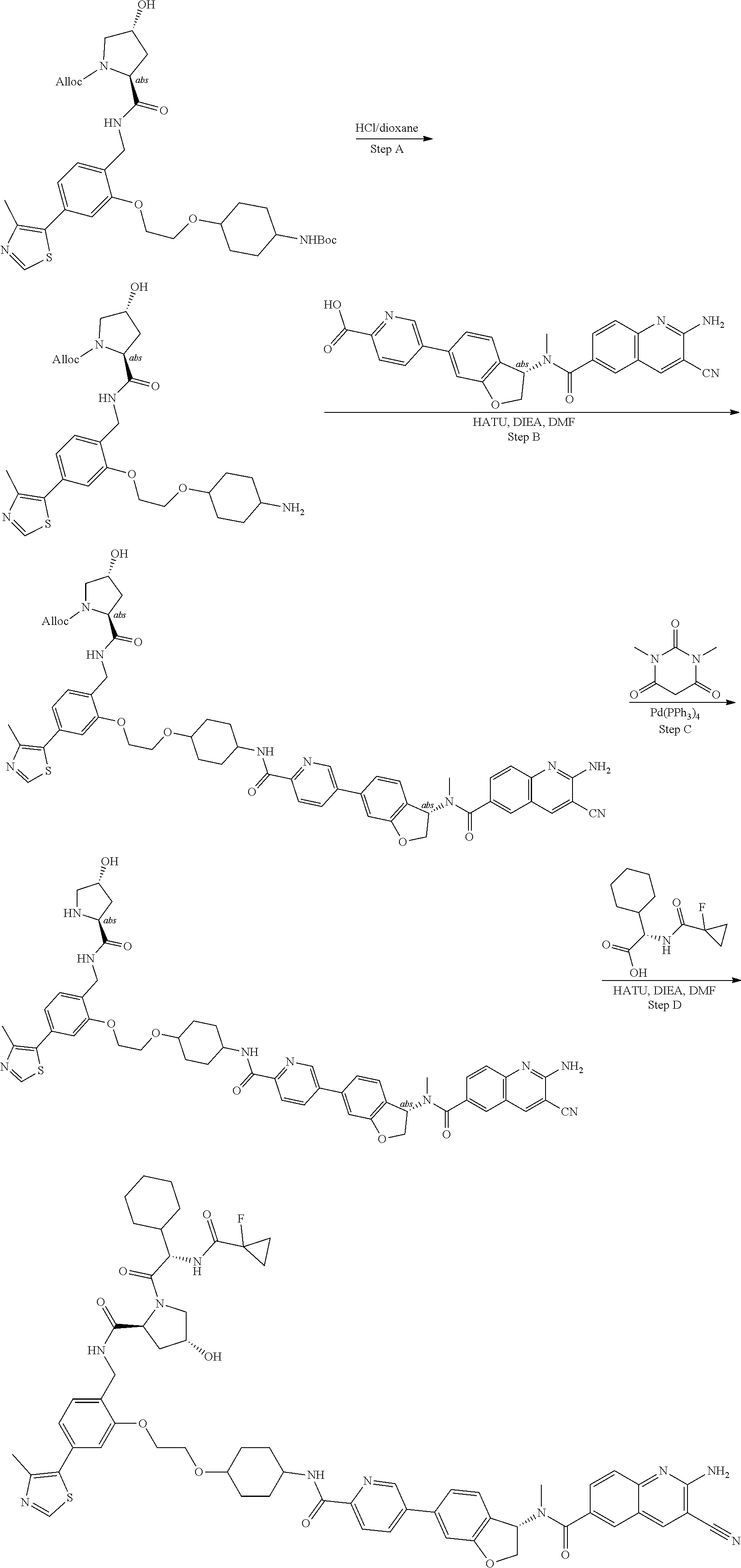

Example A389 was synthesized following the same procedures as described in Example A359 (5 mg, 4% yield over 4 steps) as a yellow solid. LC-MS (ESI): mass calcd. for C₆₂H₆₇FN₁₀O₉S, 1146.5/z found, 1148.0 [M+H]⁺. ¹H NMR (400 MHz, DMSO-d₆) δ 9.00 (s, 1H), 8.92 (s, 1H), 8.82 (s, 1H), 8.49 (d, J=8.5 Hz, 1H), 8.41-8.32 (m, 1H), 8.31-8.24 (m, 1H), 8.08 (d, J=8.1 Hz, 1H), 7.97 (s, 1H), 7.88 (d, J=7.3 Hz, 1H), 7.84-7.78 (m, 1H), 7.63-7.53 (m, 2H), 7.39 (d, J=7.6 Hz, 3H), 7.33 (d, J=7.8 Hz, 2H), 7.06 (s, 1H), 7.00 (d, J=7.7 Hz, 1H), 4.71-4.67 (m, 2H), 4.65-4.62 (m, 1H), 4.49-4.44 (m, 2H), 4.38-4.35 (m, 1H), 4.30-4.25 (m, 2H), 4.20-4.15 (m, 2H), 3.85-3.78 (m, 3H), 3.70-3.61 (m, 2H), 3.40-3.33 (m, 1H), 2.67 (s, 3H), 2.48 (s, 3H), 2.12-2.01 (m, 3H), 1.99-1.91 (m, 1H), 1.90-1.74 (m, 4H), 1.71-1.48 (m, 6H), 1.35-1.25 (m, 4H), 1.20-1.02 (m, 5H), 0.98-0.81 (m, 2H).

### Example A390. 2-amino-3-ethynyl-N-((S)-6-(6-(4-(2-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl) acetyl)piperazine-1-carbonyl)pyridin-3-yl)-2,3-dihydrobenzofuran-3-yl)-N-methylquinoline-6-carboxamide

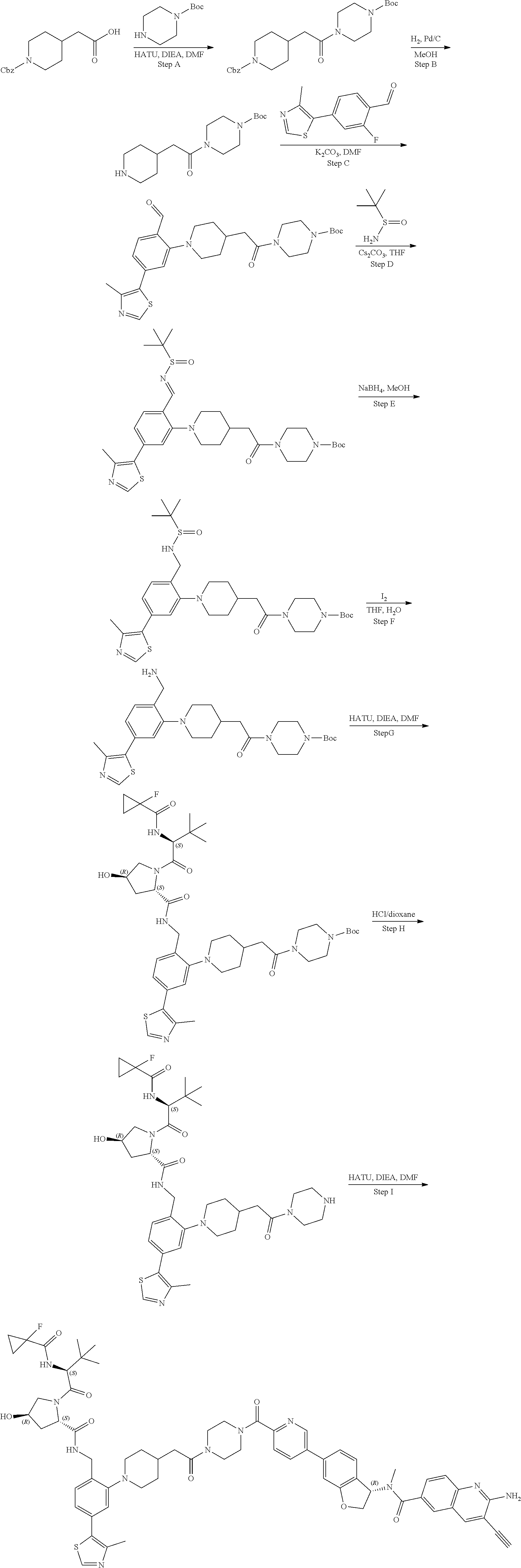

Step A: methyl (E)-4-bromo-2-(2-ethoxyvinyl)benzoate

To a solution of 2-(1-((benzyloxy)carbonyl)piperidin-4-yl) acetic acid (2.00 g, 7.21 mmol, 1.0 eq.) in DMF (20.0 mL) was added HATU (5.48 g, 14.4 mmol, 2.0 eq.), tert-butyl piperazine-1-carboxylate (1.34 g, 7.21 mmol, 1.0 eq.) and DIEA (1.86 g, 2.51 mL, 14.4 mmol, 2.0 eq.). The mixture was stirred at room temperature for 1 hour. The mixture was quenched water, extracted with EtOAc (100 mL×3) and the layers were separated. The organic layer was washed with brine (3×10⁰ mL), dried over Na₂SO₄, filtered and concentrated. The product was purified by flash chromatography (100-200 mesh silica gel, 0%-100% EtOAc in PE) to afford tert-butyl 4-(2-(1-((benzyloxy)carbonyl)piperidin-4-yl) acetyl)piperazine-1-carboxylate (2.20 g, 95% yield) as a yellow solid. LC-MS (ESI): mass calcd. For C₁₂H₁₃BrO₃, 284.00; m/z found, 285 [M+H]⁺.

Step B: methyl 4-bromo-2-(2-oxoethyl)benzoate

A mixture of tert-butyl 4-(2-(1-((benzyloxy)carbonyl)piperidin-4-yl) acetyl)piperazine-1-carboxylate (2.68 g, 6.01 mmol, 1.0 eq.), Pd/C (200 mg, 10% w.t) and Pd (OH) 2 (200 mg, 10% w.t) in MeOH (50 mL) was stirred at 50° C. for 16 hour under nitrogen. The mixture was removed under reduced pressure to afford tert-butyl 4-(2-(piperidin-4 yl) acetyl)piperazine-1-carboxylate (1.80 g, 96% yield) as a white solid. LC-MS (ESI): mass calcd. For C₁₀H₉BrO₃, 255.97; m/z found, 257 [M+H]⁺.

Steps C-I: 2-amino-3-ethynyl-N-(S)-6-(6-(4-(2-(1-(2-(((28,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl) acetyl)piperazine-1-carbonyl)pyridin-3-yl)-2,3-dihydrobenzofuran-3-yl)-N-methylquinoline-6-carboxamide

Example A390 was synthesized following the same procedures as described in Example A107 (3.8 mg, 1% yield over 7 steps) as a white solid. LC-MS (ESI): mass calcd. for C₆₄H₇₀FN₁₁O₈S, 1171.51; m/z found, 1172 [M+H]⁺. ¹H NMR (400 MHz, DMSO-d₆) δ 8.96 (d, J==27.3 Hz, 2H), 8.60 (d, J=69.9 Hz, 3H), 8.23 (s, 1H), 8.07 (s, 1H), 7.90 (s, 1H), 7.71 (s, 2H), 7.55 (d, J=7.5 Hz, 1H), 7.38 (d, J=23.5 Hz, 3H), 7.27 (s, 1H), 7.15 (s, 2H), 4.97 (s, 1H), 4.69 (s, 2H), 4.60 (s, 1H), 4.47 (s, 1H), 4.36 (s, 3H), 3.76 (s, 10H), 3.08 (s, 3H), 2.67 (s, 3H), 2.46 (s, 3H), 2.33 (s, 2H), 2.07 (s, 2H), 1.87 (d, J=32.3 Hz, 4H), 1.37 (s, 4H), 1.23 (s, 3H), 0.96 (s, 9H).

### Example A391. 4-amino-N-((S)-6-(4-((4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy) bicyclo[2.2.2]octan-1-yl)carbamoyl)phenyl)-2,3-dihydrobenzofuran-3-yl)-N-methyl-1,3-dihydrofuro[3,4-c][1,7]naphthyridine-8-carboxamide

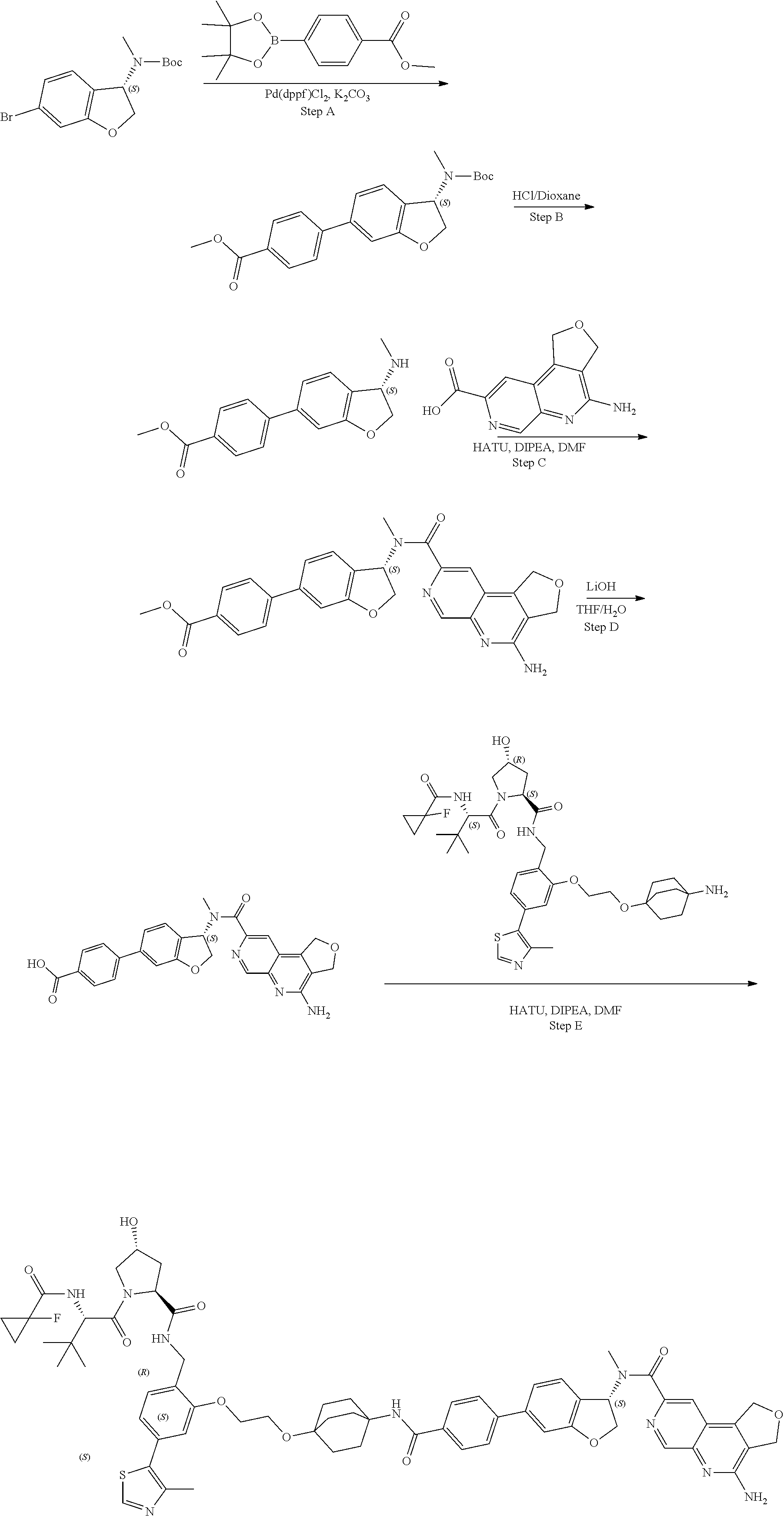

Example A391 was synthesized following the same procedures as described in Example A9 (3.8 mg, 8% yield over 5 steps) as a white solid. LC-MS (ESI): mass calcd, for C₆₃H₇₀FN₉O₁₀S, 1163.50 m/z found, 1164.8 [M+H]⁺. ¹H NMR (400 MHz, DMSO-d₆) δ 9.02 (s, 1H), 8.99 (s, 1H), 8.49 (t, J=5.8 Hz, 1H), 8.07 (s, 1H), 7.96 (d, J=19.6 Hz, 1H), 7.85 (d, J=8.1 Hz, 2H), 7.72 (d, J=4.2 Hz, 3H), 7.50 (dd, J=45.0, 7.6 Hz, 1H), 7.40 (d, J=7.9 Hz, 1H), 7.31 (dd, J=15.0, 7.2 Hz, 2H), 7.23 (d, J=12.7 Hz, 1H), 7.03 (s, 1H), 6.96 (d, J=8.8 Hz, 1H), 5.44 (s, 2H), 5.10 (s, 2H), 4.86-4.77 (m, 1H), 4.67 (d, J=6.4 Hz, 2H), 4.60 (d, J=9.2 Hz, 2H), 4.55-4.50 (m, 2H), 4.36 (s, 1H), 4.23 (s, 2H), 4.11 (s, 1H), 3.68 (d, J=3.8 Hz, 2H), 3.61 (d, J=10.8 Hz, 1H), 2.71 (d, J=7.9 Hz, 3H), 2.46 (s, 3H), 2.07 (d, J=4.7 Hz, 7H), 1.98-1.91 (m, 1H), 1.75 (s, 6H), 1.42-1.31 (m, 2H), 1.24-1.20 (m, 2H), 0.96 (s, 9H).

### Example A392. 4-amino-7-fluoro-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl) carbamoyl)pyridin-3-yl)-2,3-dihydrobenzofuran-3-yl)-N-methyl-1,3-dihydrofuro[3,4-c]quinoline-8-carboxamide

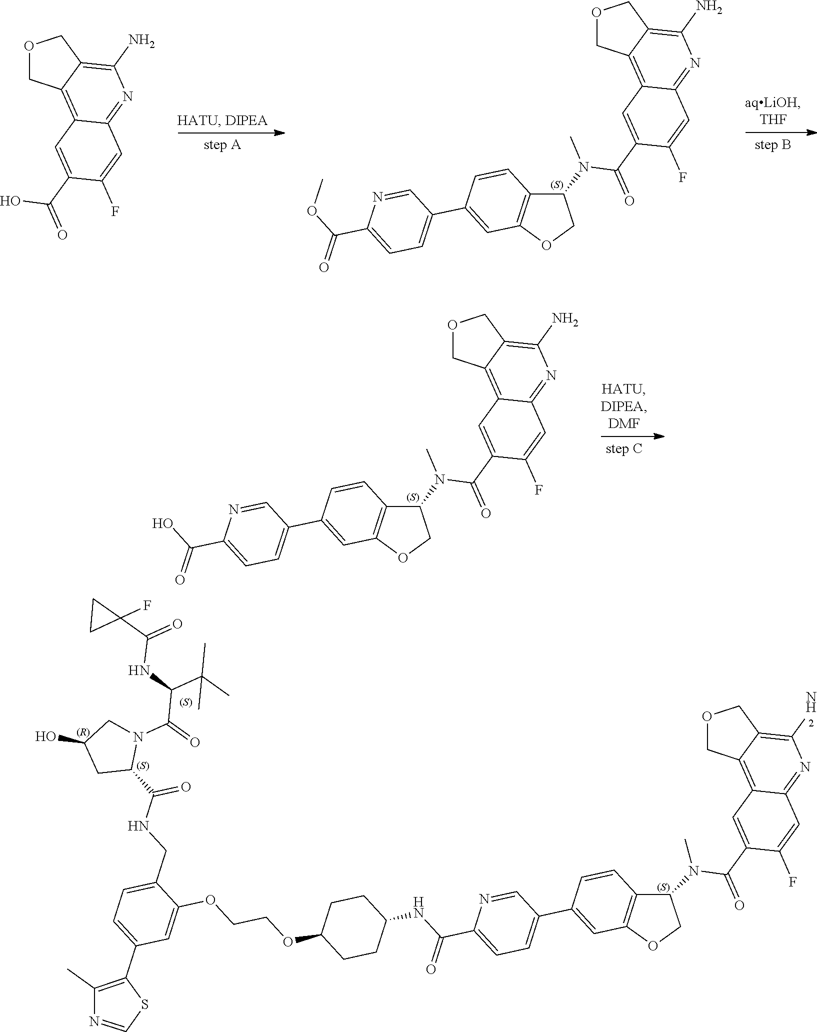

Example A392 was synthesized following the same procedures as described in Example A9 (3.4 mg, 2% yield over 3 steps) as a white solid. LC-MS (ESI): mass calcd. for C₆₁H₆₇F₂N₉O₁₀S, 1155.5/z found, 1156.8 [M+H]⁺. ¹HNMR (400 MHz, DMSO-d₆) δ 8.99 (s, 1H), 8.92 (d, J=8.2 Hz, 1H), 8.85 (s, 2H), 8.52-8.45 (m, 2H), 8.30-8.24 (m, 1H), 8.14-7.93 (m, 2H), 7.69-7.61 (m, 1H), 7.50 (d, J=7.7 Hz, 1H), 7.41 (d, J=7.8 Hz, 2H), 7.35 (d, J=19.6 Hz, 1H), 7.29 (d, J=6.7 Hz, 1H), 7.05 (s, 1H), 6.97 (d, J=7.8 Hz, 1H), 6.47 (dd, J=9.1, 3.7 Hz, 1H), 5.50-5.44 (m, 2H), 5.09 (d, J=3.7 Hz, 2H), 4.91-4.78 (m, 1H), 4.68-4.64 (m, 1H), 4.60 (d, J=9.1 Hz, 1H), 4.53 (t, J=8.2 Hz, 1H), 4.37-4.35 (m, 1H), 4.27 (dd, J=19.6, 5.7 Hz, 2H), 4.18 (s, 2H), 3.66-3.59 (m, 5H), 3.39-3.34 (m, 1H), 2.73 (s, 2H), 2.57 (s, 2H), 2.47 (s, 3H), 2.12-2.02 (m, 3H), 1.97-1.91 (m, 1H), 1.89-1.82 (m, 2H), 1.56-1.46 (m, 2H), 1.40-1.29 (m, 4H), 1.24-1,20 (m, 2H), 0.96 (s, 9H).

### Example A393. 4-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-1,3-dihydrofuro[3,4-c]quinoline-8-carboxamide

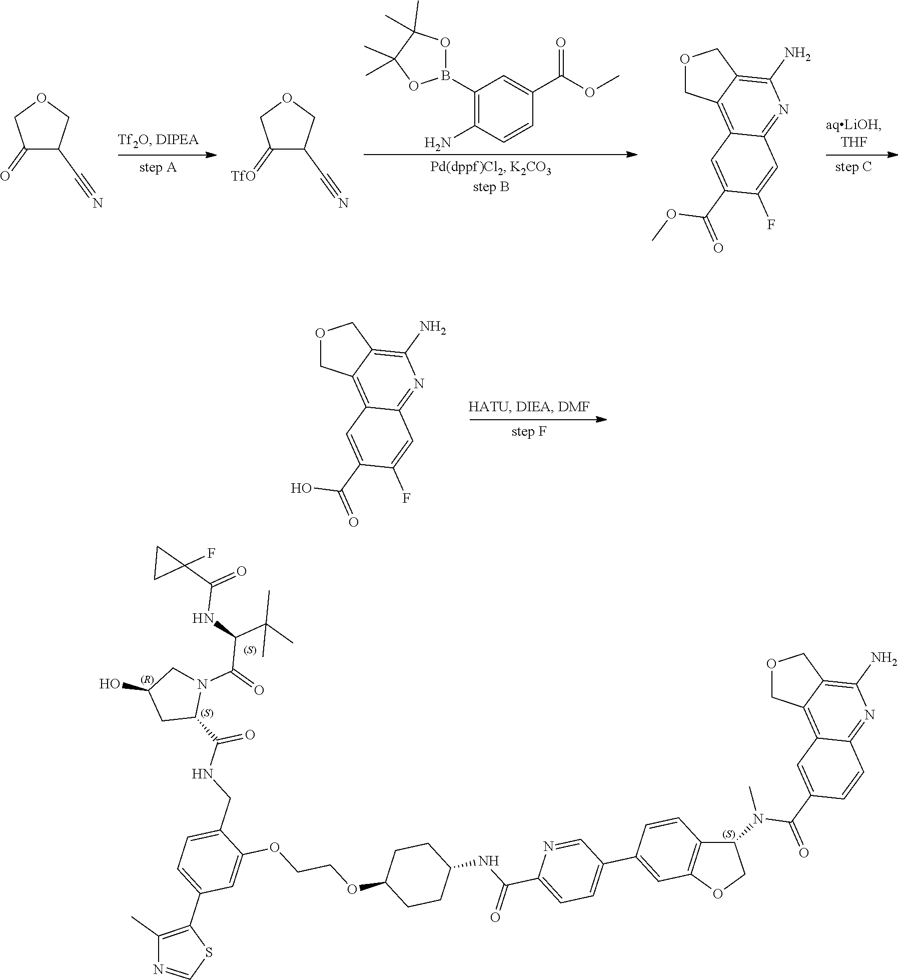

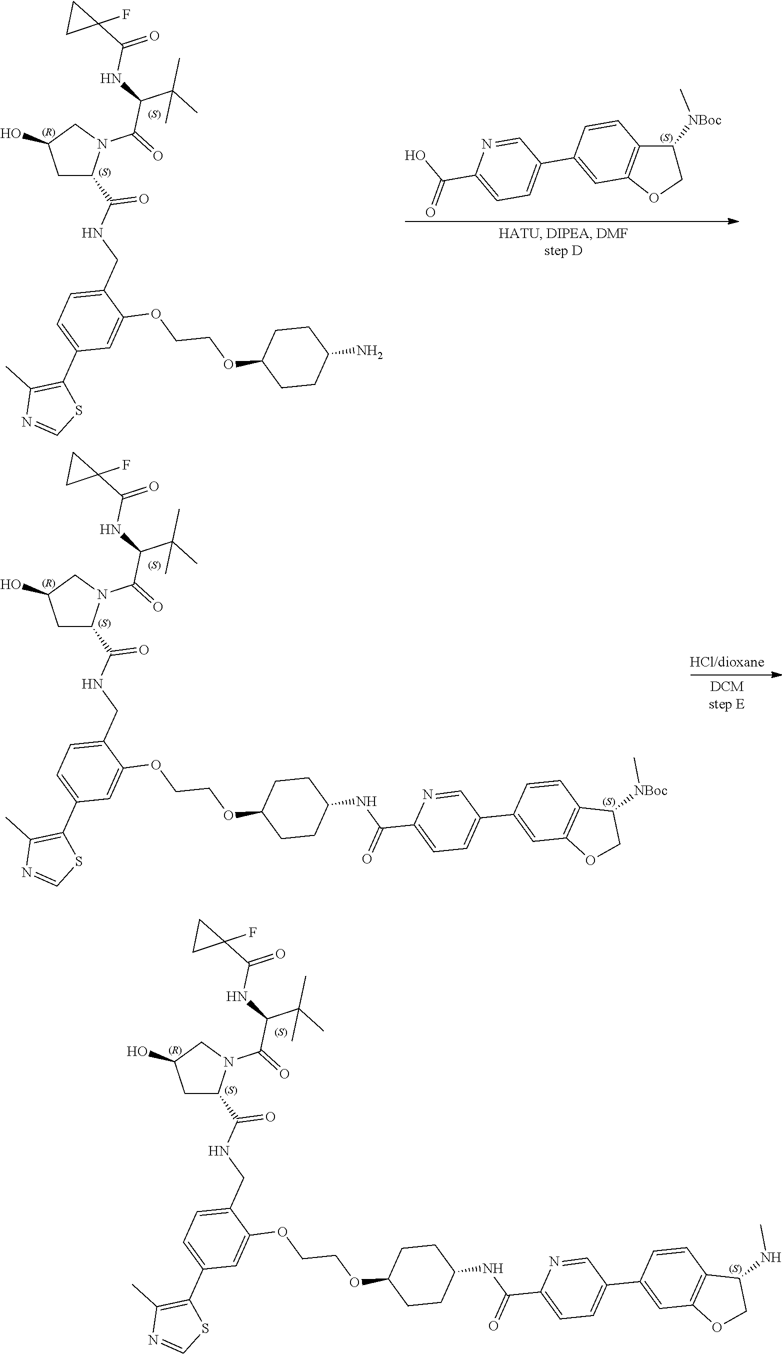

Example A393 was synthesized following the same procedures as described in Example A9 (7.8 mg, 1% yield over 6 steps) as a white solid. LC-MS (ESI): mass calcd, for C₆₁H68FN₉O₁₀S, 1137.5/z found, 1138.9 [M+H]⁺. ¹HNMR (400 MHz, DMSO-d₆) δ 8.93 (s, 1H), 8.83 (s, 2H), 8.58-8.40 (m, 2H), 8.33-8.18 (m, 2H), 8.15-7.97 (m, 2H), 7.86 (d, J=8.3 Hz, 1H), 7.81-7.68 (m, 1H), 7.55 (d, J=7.0 Hz, 1H), 7.46-7.33 (m, 2H), 7.31-7.19 (m, 2H), 7.18-6.74 (m, 2H), 4.86-4.65 (m, 2H), 4.59 (d, J=9.0 Hz, 1H), 4.49 (t, J=8.2 Hz, 1H), 4.35 (s, 1H), 4.28-4.06 (m, 5H), 3.78 (s, 4H), 3.42-3.29 (m, 2H), 2.66 (s, 3H), 2.09-2.01 (m, 5H), 1.99-1.80 (m, 4H), 1.56-1.46 (m, 2H), 1.41-1.28 (m, 4H), 1.27-1.18 (m, 3H), 1.14-1.06 (m, 2H), 0.95 (s, 9H), 0.86-0.74 (m, 2H).

### Example A394. 2-amino-3-cyano-N-((S)-6-(6-((4-(2-(2-(((2S,4R)-1-((S)-3-ethyl-2-(1-fluorocyclopropane-1-carboxamido) pentanoyl)-4-hydroxypyrrolidine-2-carboxamido)methyl)-5-(4-methylthiazol-5-yl)phenoxy)ethoxy)cyclohexyl)carbamoyl)

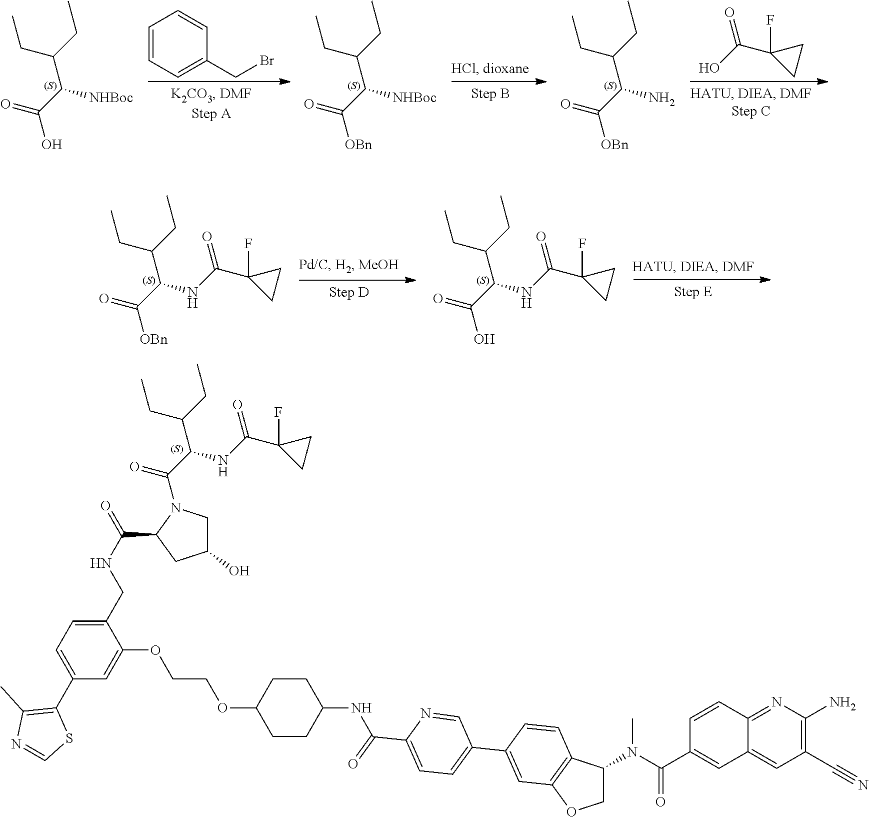

Example A394 was synthesized following the same procedures as described in Example A379 (4.6 mg, 6% yield over 5 steps) as a white solid. LC-MS (ESI): mass calcd. for C₆₁H₆₇FN₁₀O₉S, 1134.48/z found, 1135.6 [M+H]⁺. ¹H NMR (400 MHz, DMSO-d₆) δ 9.00 (s, 1H), 8.92 (s, 1H), 8.83 (s, 1H), 8.49 (d, J=8.4 Hz, 1H), 8.36 (t, J=5.8 Hz, 1H), 8.28 (dd, J=8.2, 2.2 Hz, 1H), 8.08 (d, J=8.3 Hz, 1H), 7.98 (s, 1H), 7.89 (d, J=8.1 Hz, 1H), 7.82 (dd, J=8.7, 1.9 Hz, 1H), 7.69 (d, J=6.8 Hz, 1H), 7.64-7.51 (m, 3H), 7.39 (d, J=7.8 Hz, 1H), 7.35-7.30 (m, 2H), 7.06 (s, 1H), 7.01-6.97 (m, 1H), 4.75-4.68 (m, 2H), 4.49 (t, J=8.0 Hz, 1H), 4.44-4.41 (m, 1H), 4.40-4.36 (m, 1H), 4.28 (d, J=5.7 Hz, 2H), 4.20-4.15 (m, 2H), 3.83-3.79 (m, 4H), 2.67 (s, 3H), 2.47 (s, 3H), 2.08-1.84 (m, 6H), 1.78-1.72 (m, 2H), 1.32-1.27 (m, 8H), 1.20-1.16 (m, 4H), 0.88-0.83 (m, 9H).

### Example A395. 2-amino-3-cyano-N-((S)-6-(6-((4-(2-(2-(((2S,4R)-1-((S)-2-(1-fluorocyclopropane-1-carboxamido)-2-(1-methylcyclopentyl) acetyl)-4-hydroxypyrrolidine-2-carboxamido)methyl)-5-(4-methylthiazol-5-yl)phenoxy)ethoxy)cyclohexyl)carbamoyl)

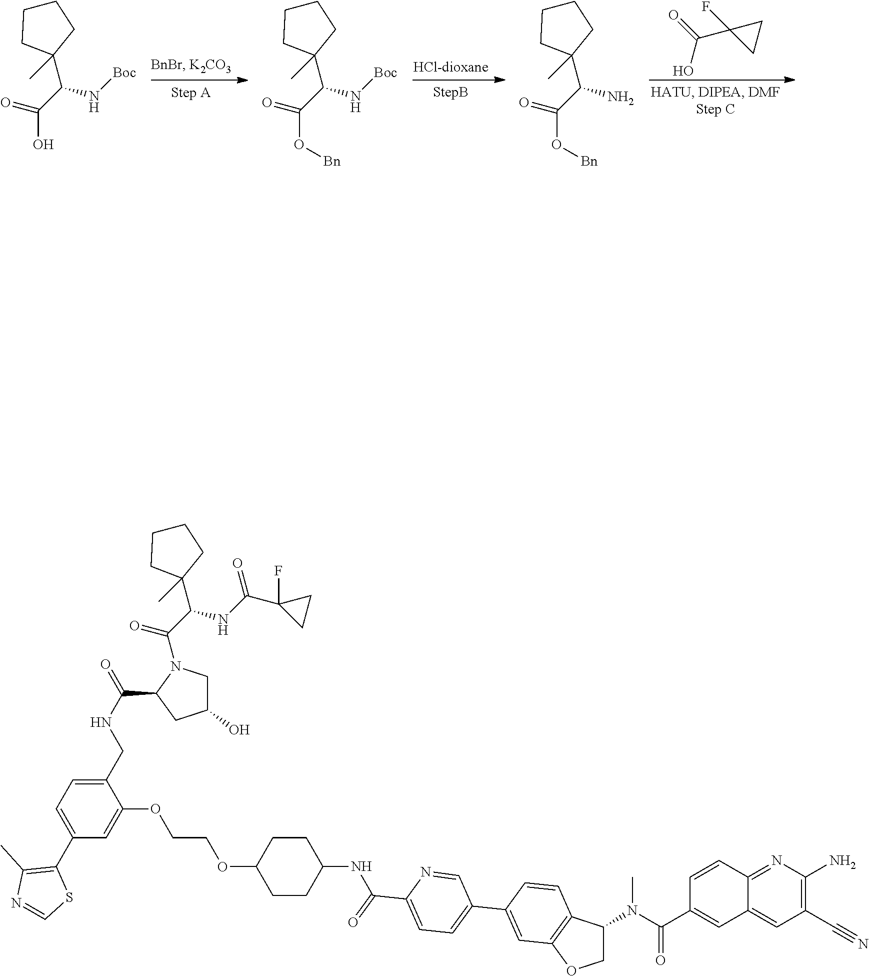 [SMILES: CC(C)(C)OC(=O)N[C@H](C(=O)O)C1(C)CCCC1 ; CC(C)(C)OC(=O)N[C@H](C(=O)OCc1ccccc1)C1(C)CCCC1 ; CC1([C@H](N)C(=O)OCc2ccccc2)CCCC1 ; O=C(O)C1(F)CC1 ; Cc1ncsc1-c1ccc(CNC(=O)[C@@H]2C[C@@H](O)CN2C(=O)[C@@H](NC(=O)C2(F)CC2)C2(C)CCCC2)c(OCCOC2CCC(NC(=O)c3ccc(-c4ccc5c(c4)OC[C@H]5N(C)C(=O)c4ccc5nc(N)c(C#N)cc5c4)cn3)CC2)c1]

Example A395 was synthesized following the same procedures as described in Example A379 (1.2 mg, 1% yield over 5 steps) as a white solid. LC-MS (ESI): mass calcd. for C₆₂H₆₇FN₁₀O₉S, 1146.5/z found, 1147.3 [M+H]⁺. ¹H NMR (400 MHz, DMSO-d₆) δ 8.99 (s, 1H), 8.93 (d, J=1.9 Hz, 1H), 8.77 (s, 1H), 8.58-8.43 (m, 2H), 8.28 (dd, J=8.2, 2.1 Hz, 1H), 8.08 (d, J=8.2 Hz, 1H), 7.95 (s, 1H), 7.79 (dd, J=8.7, 1.9 Hz, 1H), 7.56 (d, J=7.8 Hz, 2H), 7.42-7.37 (m, 2H), 7.34 (s, 1H), 7.18 (d, J=9.4 Hz, 2H), 7.06 (d, J=7.7 Hz, 1H), 6.99 (dd, J=15.5, 8.3 Hz, 1H), 5.17 (d, J=3.7 Hz, 1H), 4.63 (ddd, J=74.7, 14.3, 8.9 Hz, 3H), 4.39-4.28 (m, 2H), 4.24 (t, J=8.2 Hz, 1H), 4.17 (s, 2H), 3.80 (d, J=3.5 Hz, 3H), 3.59 (d, J=9.7 Hz, 1H), 3.36 (s, 2H), 3.31 (s, 2H), 2.68 (s, 3H), 2.47 (d, J=3.0 Hz, 3H), 2.05 (d, J=9.1 Hz, 3H), 1.99-1.82 (m, 4H), 1.73-1.60 (m, 2H), 1.50 (d,)=26.1 Hz, 6H), 1.40-1.17 (m, 10H).

### Example A396. 4-amino-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-1H-pyrazolo[4,3-c]quinoline-8-carboxamide

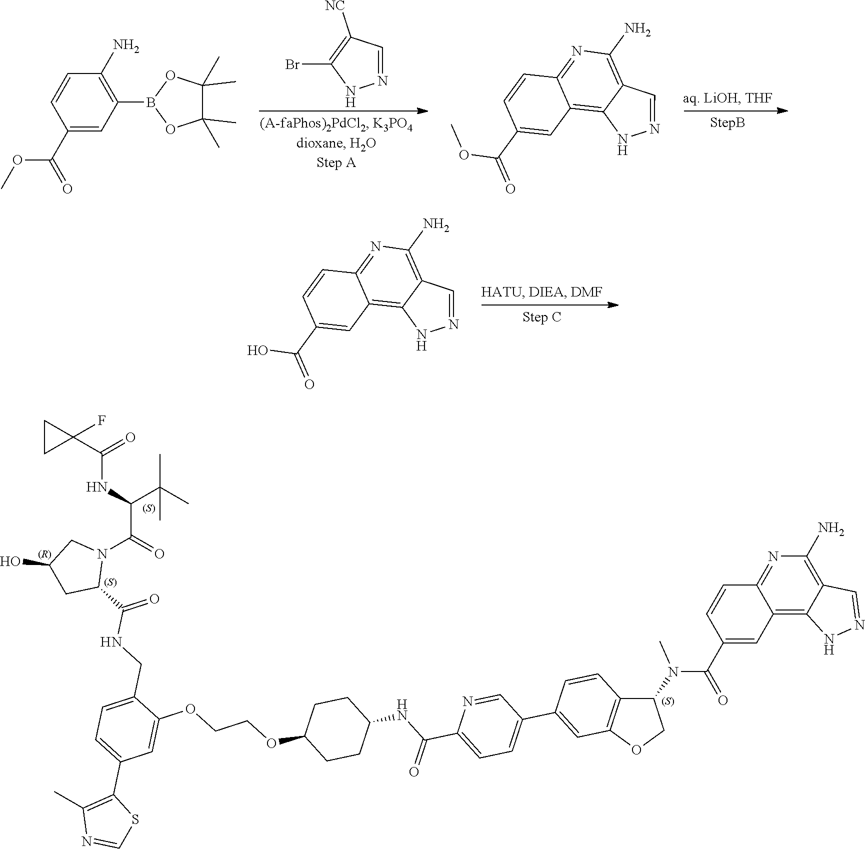

Step A: methyl(S)-3-(4-bromophenyl)-3-((tert-butoxycarbonyl)amino)propanoate

To a stirred mixture of methyl 4-amino-3-(4,4,5,5-tetramethyl-1,3,2-dioxaborolan-2-yl)benzoate (1 g, 3.61 mmol, 1.0 eq.),Bis(di-tert-butyl (4-dimethylaminophenyl)phosphine)dichloropalladium (II)(232 mg, 0.33 mmol, 0.1 eq.) and 5-bromo-1H-pyrazole-4-carbonitrile (564 mg, 3.28 mmol, 1.0 eq.) in 1,4-Dioxane (8 mL) and H₂O (2 mL) was added Potassium phosphate, tribasic (2.1 g, 9.84 mmol, 3.0 eq.). The resulting mixture was stirred at 90° C. for 15 hours. The reaction mixture was quenched with H₂O (20 mL), and then extracted with EtOAc (50 mL×2). The combined organic layers were washed with brine, dried over anhydrous Na₂SO₄ and concentrated. The residue was purified by column chromatography (silica gel, 0% EtOAc in PE~20% EtOAc in PE) to afford methyl 4-amino-1H-pyrazolo[4,3-c]quinoline-8-carboxylate (30 mg, 3.8% yield) as a white solid. LC-MS (ESI): mass calcd. For C₁₂H₁₀N₄O₂, 242.08; m/z found, 243.2 [M+H]⁺.

Steps B-C: 4-amino-N-(S)-6-(6-(((15, 4S)-4-(2-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methyl-1H-pyrazolo[4,3-c]quinoline-8-carboxamide

Example A396 was synthesized following the same procedures as described in Example A29 (14.2 mg, 5% yield over 2 steps) as a white solid. LC-MS (ESI): mass calcd. for C₆₀H₆₆FN₁₁O₉S, 1135.5/z found, 1136.3 [M+H]⁺, ¹H NMR (400 MHz, DMSO-d₆) δ 8.99 (s, 1H), 8.93 (s, 1H), 8.74 (s, 1H), 8.54-8.38 (m, 3H), 8.28 (d, J=8.1 Hz, 1H), 8.09 (d, J=8.1 Hz, 1H), 7.95-7.72 (m, 2H), 7.58 (d, J=7.8 Hz, 1H), 7.41 (d, J=7.7 Hz, 2H), 7.36 (s, 1H), 7.29 (d, J=6.8 Hz, 1H), 7.06 (s, 1H), 6.97 (d, J=7.7 Hz, 1H), 4.72 (d, J=7.0 Hz, 1H), 4.60 (d, J=9.0 Hz, 1H), 4.52 (t, J=8.2 Hz, 1H), 4.38-4.29 (m, 2H), 4.28-4.21 (m, 1H), 4.20-4.16 (m, 2H), 3.84-3.79 (m, 3H), 3.68-3.64 (m, 2H), 3.39-3.34 (m, 2H), 2.74-2.66 (m, 3H), 2.47 (s, 3H), 2.10-2.01 (m, 3H), 1.98-1.91 (m, 1H), 1.89-1.82 (m, 2H), 1.57-1.47 (m, 2H), 1.41-1.29 (m, 4H), 1.27-1.17 (m, 3H), 0.96 (s, 9H).

### Example A397. 4-amino-N-((S)-6-(6-(((1S,4S)-4-(2-(2-(((2S,4R)-1-((S)-2-cyclopentyl-2-(1-fluorocyclopropane-1-carboxamido) acetyl)-4-hydroxypyrrolidine-2-carboxamido)methyl)-5-(4-methylthiazol-5-yl)phenoxy)ethoxy)cyclohexyl)carbamoyl)pyridin-3-yl)-2,3-dihydrobenzofuran-3-yl)-N-methyl-1,3-dihydrofuro[3,4-c][1,7]naphthyridine-8-carboxamide

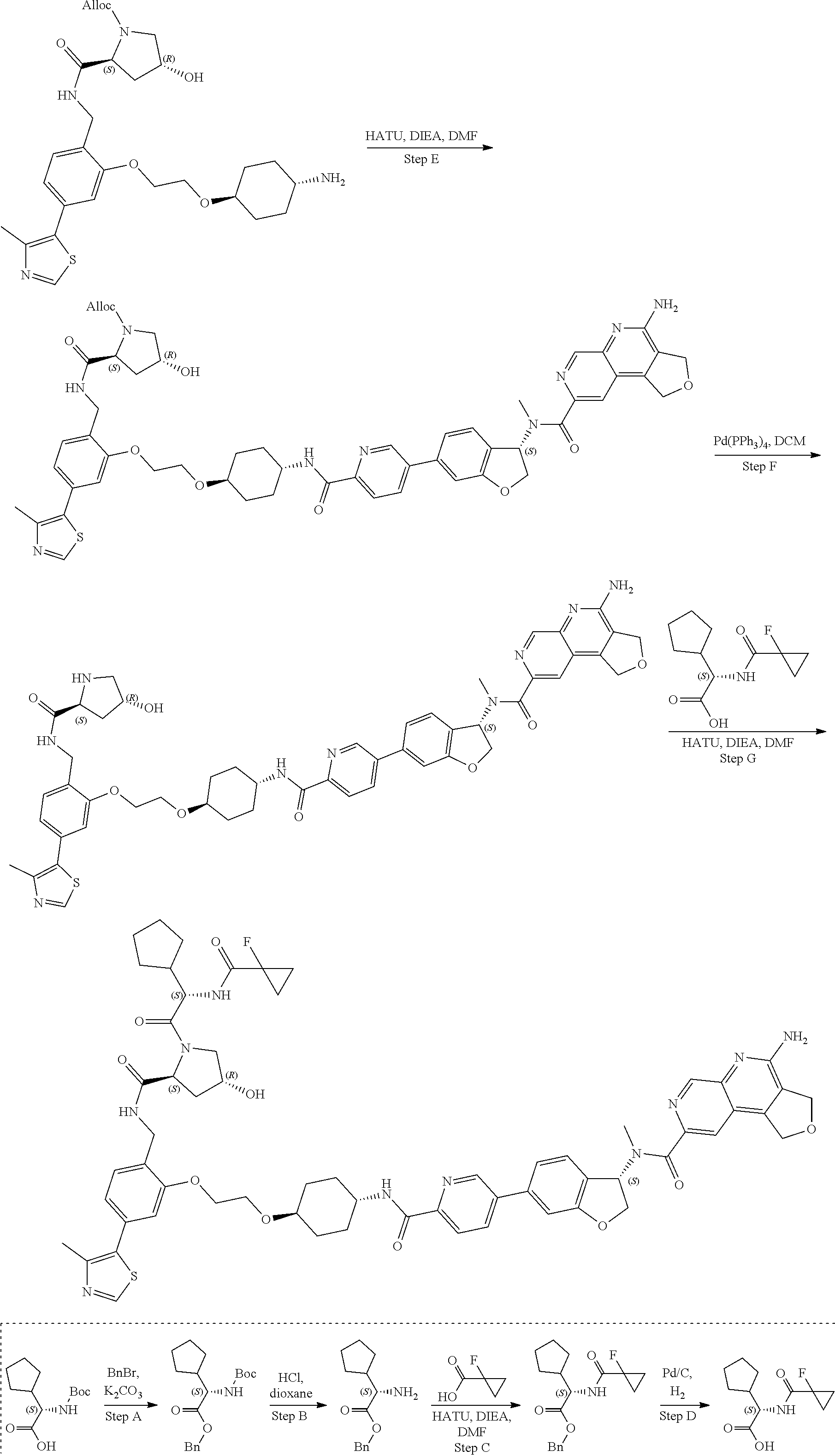

Example A397 was synthesized following the same procedures as described in Example A379 (10.0 mg, 32% yield over 7 steps) as a white solid. LC-MS (ESI): mass calcd. for C₆₁H₆₇FN₁₀O₁₀S, 1150.47/z found, 1151.5 [M+H]⁺. ¹H NMR (400 MHz, DMSO-d₆) δ 9.03-8.97 (m, 2H), 8.94-8.91 (m, 1H), 8.49 (d, J=8.5 Hz, 1H), 8.36-8.31 (m, 1H), 8.29-8.25 (m, 1H), 8.09-7.94 (m, 4H), 7.64-7.49 (m, 1H), 7.42-7.38 (m, 1H), 7.37-7.32 (m, 2H), 7.07-7.05 (m, 1H), 7.01-6.97 (m, 1H), 5.46-5.42 (m, 2H), 5.11-5.07 (m, 2H), 4.72-4.63 (m, 2H), 4.54-4.46 (m, 2H), 4.38-4.35 (m, 1H), 4.28 (d, J=5.8 Hz, 2H), 4.19-4.16 (m, 2H), 3.82-3.80 (m, 6H), 3.40-3.33 (m, 2H), 2.73-2.70 (m, 3H), 2.47 (s, 3H), 2.34-2.30 (m, 1H), 2.08-2.02 (m, 3H), 1.98-1.93 (m, 1H), 1.88-1.83 (m, 2H), 1.69-1.62 (m, 2H), 1.55-1,47 (m, 4H), 1.44-1.38 (m, 2H), 1.33-1.18 (m, 8H).

### Example A398. 2-amino-3-ethynyl-N-methyl-N-((3S)-6-(6-(4-(2,2,2-trifluoro-1-(1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)ethyl)piperazine-1-carbonyl)pyridin-3-yl)-2,3-dihydrobenzofuran-3-yl)quinoline-6-carboxamide

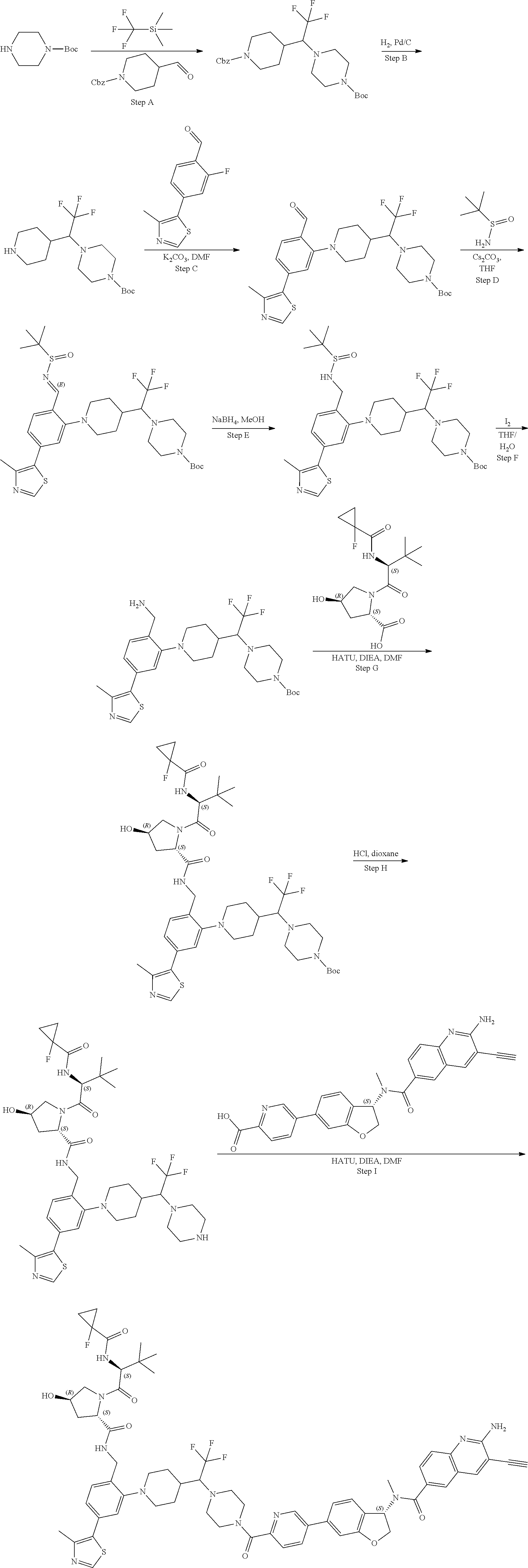

Step A: tert-butyl 4-(1-(1-((benzyloxy)carbonyl)piperidin-4-yl)-2,2,2-trifluoroethyl)piperazine-1-carboxylate

To a solution of tert-butyl piperazine-1-carboxylate (6 g, 32.2 mmol, 1.0 eq.) in MeCN (60 mL) was added benzyl 4-formylpiperidine-1-carboxylate (7.97 g, 32.2 mmol, 1.0 eq.), trimethyl(trifluoromethyl) silane (18.3 g, 129 mmol, 4.0 eq.), TFA (5.51 g, 48.3 mmol, 1.5 eq.) and KHF₂ (5.03 g, 64.4 mmol, 2.0 eq.) at room temperature. The mixture was then stirred at room temperature for 2 hours under N₂ atmosphere. The resulting solution was diluted with 30 mL of H₂O and extracted with EtOAc (30 mL×2). The combined organic layers were washed with brine, dried over anhydrous Na₂SO₄ and concentrated under reduced pressure. The residue was purified by column chromatography (silica gel, 0% EtOAc in PE~4% EtOAc in PE) to afford tert-butyl 4-(1-(1-((benzyloxy)carbonyl)piperidin-4-yl)-2,2,2-trifluoroethyl)piperazine-1-carboxylate (150 mg, 1% yield) as a white solid. LC-MS (ESI): mass calcd. For C₂₄H₃₄F₃N₃O₄, 485.3; m/z found, 486.5 [M+H]⁺.

Step B: tert-butyl 4-(2,2,2-trifluoro-1-(piperidin-4-yl)ethyl)piperazine-1-carboxylate

To a solution of tert-butyl 4-(1-(1-((benzyloxy)carbonyl)piperidin-4-yl)-2,2,2-trifluoroethyl)piperazine-1-carboxylate (150 mg, 309 μmol, 1.0 eq.) in TFE (5 mL) was added Palladium (2+) dihydroxide (30 mg, 214 μmol, 0.7 eq.) and Pd/C (30 mg, 282 μmol, 0.9 eq.) at room temperature. The mixture was then stirred at room temperature for 16 hours under H₂ atmosphere. The solid was filtered out. The filtrate was concentrated under reduced pressure to afford tert-butyl 4-(2,2,2-trifluoro-1-(piperidin-4-yl)ethyl)piperazine-1-carboxylate (100 mg, 92% yield) as a colourless oil. LC-MS (ESI): mass calcd. For C₁₆H₂₈F₃N₃O₂, 351.2; m/z found, 352.3 [M+H]⁺.

Steps C-I: 2-amino-3-ethynyl-N-methyl-N-(3S)-6-(6-(4-(2,2,2-trifluoro-1-(1-(2-(((2S,4R)-1-(S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)ethyl)piperazine-1-carbonyl)pyridin-3-yl)-2,3-dihydrobenzofuran-3-yl)quinoline-6-carboxamide

Example A398 was synthesized following the same procedures as described in Example A107 (11 mg, 1% yield over 7 steps) as a white solid. LC-MS (ESI): mass calcd. for C₆₄H₆₉F₄N₁₁O₇S, 1211.5/z found, 1212.7 [M+H]⁺. ¹H NMR (400 MHz, DMSO-d₆) δ 9.00 (s, 1H), 8.93 (s, 1H), 8.71 (s, 2H), 8.57 (s, 1H), 8.27-8.16 (m, 1H), 8.09 (s, 1H), 7.97-7.88 (m, 1H), 7.78 (s, 1H), 7.67 (d, J=8.2 Hz, 1H), 7.55 (d, J=7.8 Hz, 1H), 7.47-7.43 (m, 1H), 7.40 (d, J=6.9 Hz, 1H), 7.34 (s, 1H), 7.27 (d, J=9.2 Hz, 1H), 7.17 (s, 2H), 4.99 (s, 1H), 4.70-4.66 (m, 1H), 4.62-4.58 (m, 1H), 4.51-4.46 (m, 1H), 4.45-4.39 (m, 1H), 4.37-4.31 (m, 2H), 3.71-3.59 (m, 4H), 3.49-3.41 (m, 2H), 3.40-3.27 (m, 2H), 3.19-3.10 (m, 2H), 3.04-2.67 (m, 7H), 2.67 (s, 3H), 2.47 (s, 3H), 2.18-2.07 (m, 2H), 1.95-1.87 (m, 2H), 1.85-1.78 (m, 1H), 1.63-1.49 (m, 2H), 1.40-1.32 (m, 2H), 1.24-1.20 (m, 2H), 0.97 (s, 9H).

### Example A399. N1-(7-amino-1-methyl-1H-pyrazolo[3,4-c]pyridin-4-yl)-N2-((S)-6-(6-(4-((1-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenyl)piperidin-4-yl)methyl)piperazine-1-carbonyl)pyridin-3-yl)-2,3-dihydrobenzofuran-3-yl)-N2-methyloxalamide

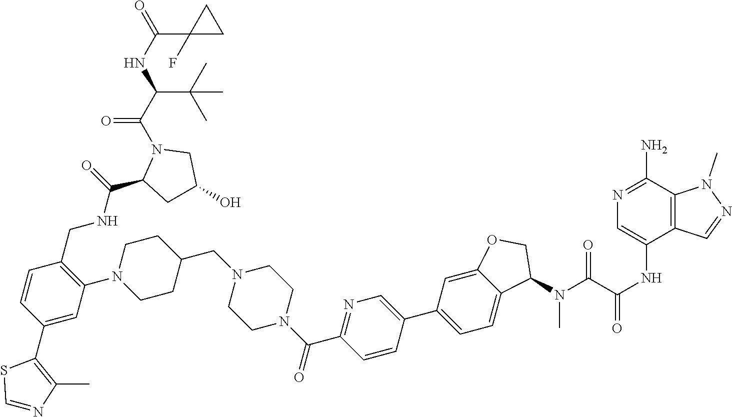 [SMILES: Cc1ncsc1-c1ccc(CNC(=O)[C@@H]2C[C@@H](O)CN2C(=O)[C@@H](NC(=O)C2(F)CC2)C(C)(C)C)c(N2CCC(CN3CCN(C(=O)c4ccc(-c5ccc6c(c5)OC[C@H]6N(C)C(=O)C(=O)Nc5cnc(N)c6c5cnn6C)cn4)CC3)CC2)c1]

Example A399 was synthesized following the same procedures as described in Example A93. LC-MS (ESI): mass calcd, for C₆₀H₆₁FN1408S, 1166.53; m/z found, 1167 [M+H]⁺. ¹H NMR (400 MHz, DMSO-d₆) δ 8.99 (s, 1H), 8.96-8.92 (m, 1H), 8.52 (t, J=5.3 Hz, 1H), 8.38 (d, J=6.2 Hz, 1H), 8.30-8.26 (m, 1H), 8.11-8.02 (m, 1H), 7.80-7.74 (m, 1H), 7.55 (dd, J=44.8, 7.9 Hz, 1H), 7.46-7.41 (m, 2H), 7.38 (d, J=5.7 Hz, 1H), 7.27 (dd, J=8.9, 2.3 Hz, 1H), 7.14-7.10 (m, 2H), 6.06 (ddd, J=12.0, 8.2, 3.3 Hz, 1H), 5.19 (s, 1H), 4.79-4.66 (m, 2H), 4.60 (d, J=8.9 Hz, 2H), 4.48 (t, J=8.1 Hz, 1H), 4.37 (s, 3H), 4.35-4.33 (m, 1H), 4.21 (s, 1H), 3.69-3.59 (m, 6H), 3.19-3.04 (m, 8H), 2.76-2.63 (m, 6H), 2.46 (s, 3H), 2.10-1.97 (m, 2H), 1.95-1.89 (m, 3H), 1.51-1.42 (m, 2H), 1.41-1.38 (m, 1H), 1.36-1.33 (m, 1H), 1.24-1.22 (m, 1H), 1.22-1.19 (m, 1H), 0.98 (s, 9H).

### Example A400. 3-cyclopropyl-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methyl-2-(methylamino) quinoline-6-carboxamide

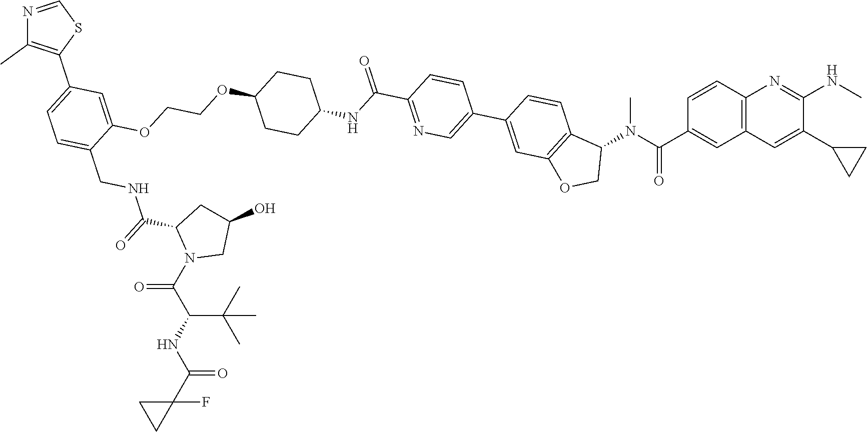 [SMILES: CNc1nc2ccc(C(=O)N(C)[C@@H]3COc4cc(-c5ccc(C(=O)N[C@H]6CC[C@H](OCCOc7cc(-c8scnc8C)ccc7CNC(=O)[C@@H]7C[C@@H](O)CN7C(=O)[C@@H](NC(=O)C7(F)CC7)C(C)(C)C)CC6)nc5)ccc43)cc2cc1C1CC1]

Example A400 was synthesized following the same procedures as described in Example A249. LC-MS (ESI): mass calcd, for C₆₃H₇₂FN9O9S, 1149.52; m/z found, 1150 [M+H]⁺. ¹H NMR (400 MHz, DMSO) δ 9.13 (s, 1H), 8.99 (s, 1H), 8.92 (s, 1H), 8.49 (t, J=8.1 Hz, 2H), 8.27 (d, J=10.0 Hz, 1H), 8.15 (s, 1H), 8.09 (d, J=8.1 Hz, 1H), 8.03 (s, 2H), 7.86 (d, J=8.8 Hz, 1H), 7.55 (d, J=7.7 Hz, 1H), 7.43-7.33 (m, 3H), 7.29 (d, J=6.6 Hz, 1H), 7.05 (s, 1H), 6.97 (d, J=7.7 Hz, 1H), 6.00 (m, 1H), 4.75 (m, 2H), 4.60 (d, J=9.1 Hz, 1H), 4.52 (t, J=8.1 Hz, 1H), 4.36 (m, 1H), 4.30 (m, 1H), 4.25 (m, 1H), 4.18 (m, 2H), 3.81 (m, 5H), 3.65 (m, 3H), 3.36 (m, 1H), 3.23 (d, J=4.7 Hz, 3H), 2.67 (m, 2H), 2.47 (s, 3H), 2.13-2.03 (m, 3H), 1.97-1.91 (m, 1H), 1.84 (m, 3H), 1.51 (m, 2H), 1.36 (m, 3H), 1.24-1.20 (m, 2H), 1.09 (m, 2H), 1.09 (m, 2H), 0.96 (s, 9H), 0.80 (m, 2H).

### Example A401. 2-amino-3-cyano-N-((S)-6-(6-((2-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethyl)-2-azaspiro[3.5]nonan-7-yl)carbamoyl)pyridin-3-yl)-2,3-dihydrobenzofuran-3-yl)-N-methylquinoline-6-carboxamide

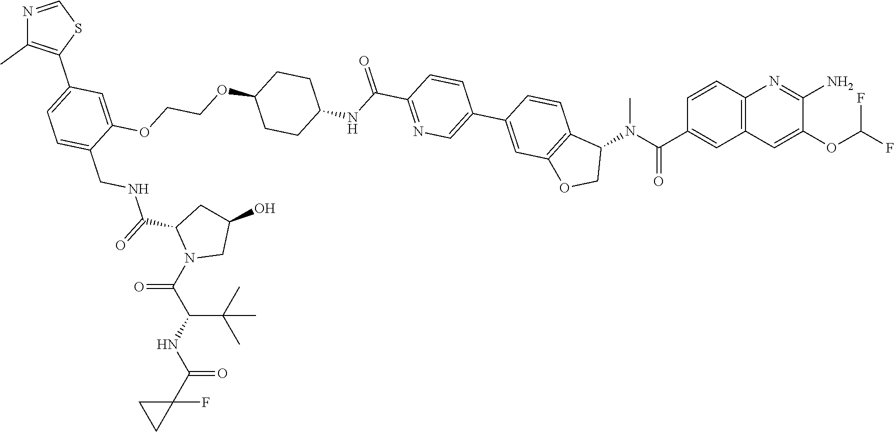 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(OC(F)F)cc5c4)cn3)CC2)c1]

Example A401 was synthesized following the same procedures as described in Example A91. LC-MS (ESI): mass calcd, for C₆₂H₆₈FN₁₁O₈S, 1145.50; m/z found, 1146 [M+H]⁺.]⁺. ¹H NMR (400 MHz, DMSO) δ 9.86 (s, 1H), 9.01 (s, 1H), 8.92 (s, 1HJ), 8.80 (s, 1H), 8.60-8.54 (m, 1H), 8.48-8.40 (m, 1H), 8.32-8.26 (m, 1H), 8.08 (d, J=8.2 Hz, 1H), 7.96 (s, 1H), 7.84-7.77 (m, 1H), 7.61-7.51 (m, 2H), 7.47-7.43 (m, 1H), 7.41-7.37 (m, 1H), 7.36-7.23 (m, 4H), 7.06-7.02 (m, 2H), 4.72-4.67 (m, 1H), 4.62-4.58 (m, 1H), 4.55-4.50 (m, 1H), 4.37-4.27 (m, 5H), 4.04-3.97 (m, 3H), 3.86-3.70 (m, 6H), 2.67 (s, 3H), 2.47 (s, 3H), 2.14-2.03 (m, 3H), 1.95-1.89 (m, 1H), 1.82-1.72 (m, 2H), 1.66-1.55 (m, 3H), 1.48-1.30 (m, 4H), 1.25-1.17 (m, 3H), 0.95 (s, 9H).

### Example A402. 2-amino-3-(difluoromethoxy)-N-((S)-6-(6-(((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)carbamoyl) pyridin-3-yl)-2,3-dihydrobenzofuran-3-yl)-N-methylquinoline-6-carboxamide

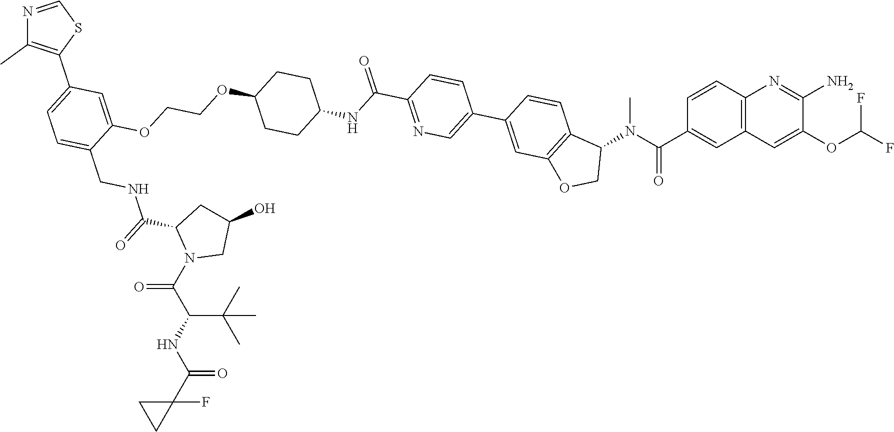 [SMILES: Cc1ncsc1-c1ccc(CNC(=O)[C@@H]2C[C@@H](O)CN2C(=O)[C@@H](NC(=O)C2(F)CC2)C(C)(C)C)c(OCCO[C@H]2CC[C@H](NC(=O)c3ccc(-c4ccc5c(c4)OC[C@H]5N(C)C(=O)c4ccc5nc(N)c(OC(F)F)cc5c4)cn3)CC2)c1]

Example A402 was synthesized following the same procedures as described in Example A248. LC-MS (ESI): mass calcd, for C₆₀H₆₆F₃N₉O₁₀S, 1161.45; m/z found, 1162 [M+H]⁺. ¹H NMR (400 MHz, DMSO) δ 8.99 (s, 1H), 8.92 (s, 1H), 8.53-8.45 (m, 2H), 8.31-8.25 (m, 1H), 8.23-8.16 (m, 1H), 8.10-8.03 (m, 2H), 7.86-7.66 (m, 2H), 7.56 (d, J=7.7 Hz, 1H), 7.44-7.34 (m, 3H), 7.32-7.25 (m, 1H), 7.05 (s, 1H), 6.97 (d, J=7.7 Hz, 1H), 4.72-4.65 (m, 1H), 4.60 (d, J=9.3 Hz, 1H), 4.52 (t, J=8.3 Hz, 1H), 4.38-4.33 (m, 1h), 4.27 (dd, J=20.1, 5.8 Hz, 2H), 4.21-4.15 (m, 3H), 3.84-3.76 (m, 4H), 3.70-3.55 (m, 2H), 3.40-3.31 (m, 1H), 2.67 (s, 3H), 2.47 (s, 3H), 2.10-2.03 (m, 2H), 2.02-1.92 (m, 2h), 1.91-1.81 (m, 2h), 1.57-1.46 (m, 2H), 1.38-1.28 (m, 4H), 1.22-1.19 (m, 2H), 0.96 (s, 9H).

### Example A403. 2-amino-3-ethynyl-N-((S)-6-(2-((1S,4S)-4-(2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)ethoxy)cyclohexyl)-1,3-dioxoisoindolin-5-yl)-2,3-dihydrobenzofuran-3-yl)-N-methylquinoline-6-carboxamide

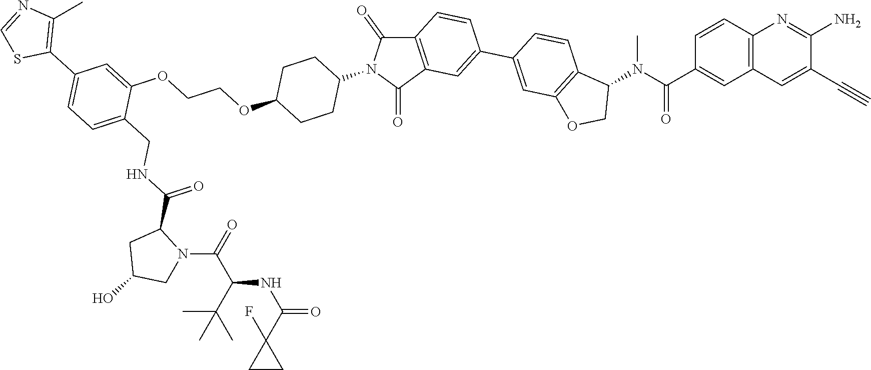

Example A403 was synthesized following the same procedures as described in Example A316. LC-MS (ESI): mass calcd, for C₆₃H₆₅FN8010S, 1144.45; m/z found, 1145 [M+H]⁺. ¹H NMR (400 MHz, DMSO) δ 8.99 (s, 1H), 8.75-8.37 (m, 4H), 8.11 (d, J=8.4 Hz, 1H), 8.09-8.05 (m, 2H), 7.90 (d, J=7.9 Hz, 2H), 7.77-7.71 (m, 1H), 7.54 (d, J=7.7 Hz, 1H), 7.45-7.40 (m, 2H), 7.36 (s, 1H), 7.32-7.27 (m, 1H), 7.07-7.05 (m, 1H), 6.98 (d, J=7.8 Hz, 1H), 4.94 (s, 1H), 4.87-4.75 (m, 1H), 4.70-4.67 (m, 1H), 4.60 (d, J=9.1 Hz, 1H), 4.52 (t, J=8.2 Hz, 1H), 4.37-4.30 (m, 2H), 4.27-4.16 (m, 4H), 4.07-4.00 (m, 2H), 3.84-3.82 (m, 2H), 3.46-3.42 (m, 3H), 2.67 (s, 3H), 2.47 (s, 3H), 2.22-2.09 (m, 5H), 1.97-1.91 (m, 1H), 1.79-1.73 (m, 2H), 1.40-1.29 (m, 4H), 1.24-1.20 (m, 2H), 0.96 (s, 9H).

### II. Biological Assessment

The Binding Potency of Representative Compounds to PRMT5-MTA and SAM were Determined Using HTRF Assay Technology.

PRMT5 binding potencies in the absence and presence of SAM or MTA were measured in a displacement assay using a CY5 labeled probe. Compound IC₅₀ values were determined for Apo PRMT5, PRMT5/MTA and PRMT5/SAM. 7.5 nL of DMSO stock of compounds were dispensed into a 384-well microplate (Perkin Elmer 6008289) with 11-point 3-fold serial dilution starting at 2 μM. 10 nL CY5 labeled probe at final concentrations of 10 nM were dispensed by ECHO to measure IC₅₀s for Apo, PRMT5/MTA and PRMT5/SAM, respectively. To a solution of 0.125 nM of anti-His Tb cryptate and 0.25 nM PRMT5/MEP50, MTA or SAM were added to a final concentration of 50 μM and the mixtures were incubated at room temperature for 30 minutes. 2 μL of the resultant solutions were added to the compound plates sealed and centrifuged at 1500G for 30 seconds. Final DMSO was 1%. The plates were incubated for 120 minutes at room temperature. The assay was run in 30 mM Bicine pH 8.0, 0.003% Tween-20, 1.5 mM DTT, 150 mM NaCl. The signal from the assay was examined on Envision (Perkin Elmer). The TR-FRET signal was calculated by determining the ratio of Emission 2 (665 nm) to Emission 1 (620 nm) following excitation at 337 nm for each data point. IC50 values were then calculated using a non-linear fit variable slope model in Signals software (PerkinElmer) and K is were calculated by using the equation IC₅₀=Kᵢ*{1+(probe/K_{d}, probe)} where K_{d} for the probe for apo, PRMT5/MTA and PRMT5/SAM were 1.3, 0.75 and 10 nM.

TABLE 2

| | MTA-PRMTS | SAM-PRMT5 |
|---|---|---|
| Compound No. | IC₅₀ (nM) | IC₅₀ (nM) |
| A1 | A | B |
| A2 | B | D |
| A4 | A | A |
| A5 | D | D |
| A6 | A | A |
| A7 | A | A |
| A8 | A | B |
| A9 | C | D |
| A11 | A | B |
| A12 | B | C |
| A13 | A | B |
| A14 | A | B |
| A15 | C | C |
| A16 | C | D |
| A17 | A | B |
| A18 | A | A |
| A19 | C | D |
| A20 | C | D |
| A21 | D | D |
| A22 | C | D |
| A24 | D | D |
| A25 | D | D |
| A26 | D | D |
| A27 | D | D |
| A28 | D | D |
| A29 | C | D |
| A30 | A | B |
| A31 | B | C |
| A32 | A | A |
| A33 | C | D |
| A34 | A | C |
| A35 | B | B |
| A36 | A | B |
| A37 | A | A |
| A38 | A | C |
| A39 | B | C |
| A40 | A | B |
| A41 | B | C |
| A42 | A | C |
| A43 | B | C |
| A44 | A | C |
| A46 | A | C |
| A47 | B | C |
| A48 | B | C |
| A49 | B | D |
| A50 | A | C |
| A51 | C | D |
| A52 | A | B |
| A53 | A | C |
| A54 | C | D |
| A55 | B | C |
| A56 | D | D |
| A57 | B | C |
| A58 | C | D |
| A59 | B | C |
| A60 | B | D |
| A61 | B | D |
| A62 | B | D |
| A63 | C | D |
| A64 | C | D |
| A65 | C | D |
| A66 | B | D |
| A67 | C | D |
| A68 | B | C |
| A69 | A | C |
| A70 | A | B |
| A71 | C | D |
| A72 | C | D |
| A73 | A | C |
| A74 | C | D |
| A81 | B | C |
| A83 | C | D |
| A86 | A | B |
| A88 | D | D |
| A89 | C | D |
| A90 | B | C |
| A91 | D | D |
| A92 | D | D |
| A93 | C | D |
| A94 | C | D |
| A95 | D | D |
| A96 | C | D |
| A97 | C | D |
| A98 | D | D |
| A99 | C | D |
| A100 | C | D |
| A101 | B | C |
| A102 | C | D |
| A103 | A | C |
| A104 | C | D |
| A105 | D | E |
| A106 | C | E |
| A107 | A | B |
| A118 | A | B |
| A109 | D | E |
| A110 | A | C |
| A111 | A | C |
| A113 | C | D |
| A114 | D | B |
| A116 | B | C |
| A117 | C | E |
| A119 | C | D |
| A120 | C | D |
| A121 | A | C |
| A122 | A | C |
| A123 | A | C |
| A124 | B | B |
| A125 | A | B |
| A126 | B | C |
| A127 | D | E |
| A128 | B | C |
| A129 | D | E |
| A130 | A | B |
| A131 | B | C |
| A132 | B | C |
| A133 | B | C |
| A134 | A | B |
| A135 | D | E |
| A136 | D | E |
| A137 | B | C |
| A145 | C | E |
| A146 | B | C |
| A147 | B | D |
| A148 | D | E |
| A149 | C | D |
| A156 | B | C |
| A157 | C | D |
| A158 | A | C |
| A159 | B | C |
| A160 | B | C |
| A162 | C | D |
| A163 | A | C |
| A164 | B | C |
| A165 | C | D |
| A166 | C | D |
| A167 | A | C |
| A168 | B | C |
| A170 | B | D |
| A171 | B | D |
| A172 | A | C |
| A173 | C | D |
| A174 | C | D |
| A175 | B | C |
| A176 | B | D |
| A177 | D | E |
| A179 | B | D |
| A180 | B | C |
| A181 | B | C |
| A182 | D | E |
| A183 | C | E |
| A185 | D | E |
| A186 | B | D |
| A188 | B | D |
| A189 | B | C |
| A190 | B | C |
| A196 | C | D |
| A199 | C | D |
| A200 | C | D |
| A206 | C | D |
| A207 | D | E |
| A209 | B | D |
| A220 | C | E |
| A221 | C | E |
| A242 | C | D |
| A248 | C | D |
| A249 | C | E |
| A255 | C | D |
| A256 | C | E |
| A257 | C | D |
| A259 | C | D |
| A260 | C | D |
| A262 | C | D |
| A265 | C | D |
| A267 | B | D |
| A268 | D | E |
| A270 | C | D |
| A272 | C | D |
| A282 | C | E |
| A283 | C | D |
| A284 | C | D |
| A285 | D | E |
| A287 | C | D |
| A288 | C | D |
| A289 | C | D |
| A295 | C | D |
| A299 | C | D |
| A303 | C | D |
| A304 | C | D |
| A306 | C | D |
| A307 | B | D |
| A311 | B | D |
| A312 | C | D |
| A313 | C | D |
| A314 | C | D |
| A321 | C | D |
| A324 | D | E |
| A325 | D | E |
| A326 | D | E |
| A327 | C | D |
| A399 | D | E |

Definitions:
IC₅₀: “A”: < 1 nM; “B”: 1-9.9 nM; “C”: 10-99 nM; “D”: 100-999 nM; “E”: >1000 nM

PRMT5 Degradation Potency and Dₘₐₓ of Representative Compounds were Determined Using in HiBit Technology in HiBit MTAP KO and MTAP WT Cells.

HCT116 cells+/−MTAP (Creative Biogene (CSC-RT2701) were genetically modified via CRISPR/Cas9 to fuse HiBIT to the carboxy terminus of PRMT5. Cells were cultured in McCoy's 5A containing 10% FBS and 1% (Vol: Vol) penicillin-streptomycin. At the time of experiment, cells were seeded at a density of 1250/25 μL/well in a 384-well plate (Corning; cat #3571) 24 hrs prior to compound treatment. Investigational compounds were prepared at 3 mM top concentration in 100% DMSO and an 11-point 3-fold serial dilution done in DMSO. 25 nL of serial diluted compounds were transferred to wells containing cell in 25 μL of media for a final top concentration of 3 μM. An equal volume of DMSO was added to control wells. Each treatment was performed in duplicate. Cells were incubated at 37° C.+5% CO₂ in a cell culture incubator for 6 hours (Examples A126 through A403 for KO cells), 18 hours (Examples A1 through A125 for KO and WT cells), or 24 hours (Examples A126 through A403 for WT cells). At the end of experiment, levels of PRMT5 were evaluated with Nano-Glo® HiBIT Lytic Detection Assay (Promega N3050). Assay reagent was prepared according to the manual provided with the Promega kit. 25 μl of this assay solution was added to each well. Luminescence from each well was measured with Envision plate reader (Perkin Elmer). Dose response curves were fit using Perkin Elmer Signals Lead Discovery software.

TABLE 3

| | MTAP KO | MTAP | MTAP WT | MTAP |
|---|---|---|---|---|
| | HiBit | KO HiBit | HiBit | WT HiBit |
| Example No. | DC₅₀ (nM) | Dₘₐₓ (%) | DC₅₀ (nM) | Dₘₐₓ (%) |
| | 18 h | | 18 h | |
| A1 | B | A | B | B |
| A2 | B | A | B | A |
| A3 | E | E | | |
| A4 | B | A | | |
| A5 | B | A | B | B |
| A6 | B | A | B | A |
| A7 | B | B | | |
| A8 | E | E | | |
| A9 | B | A | B | B |
| A10 | C | B | C | B |
| A11 | B | A | B | B |
| A12 | B | A | B | B |
| A13 | B | A | C | B |
| A14 | B | A | C | C |
| A15 | A | A | B | A |
| A16 | B | A | B | B |
| A17 | B | A | B | A |
| A18 | B | A | B | A |
| A19 | B | B | C | B |
| A20 | B | D | | |
| A21 | E | B | | |
| A22 | C | D | | |
| A23 | B | B | B | C |
| A24 | B | A | A | A |
| A25 | B | A | B | C |
| A26 | B | A | A | A |
| A27 | B | A | B | B |
| A28 | A | A | A | A |
| A29 | A | A | A | A |
| A30 | B | A | B | B |
| A31 | B | B | C | D |
| A32 | B | A | B | A |
| A33 | B | A | C | B |
| A34 | B | A | B | D |
| A35 | C | A | B | A |
| A36 | B | D | | |
| A37 | B | C | B | C |
| A38 | B | A | B | B |
| A39 | B | A | B | B |
| A40 | B | A | C | B |
| A41 | C | B | C | C |
| A42 | A | A | B | A |
| A43 | A | A | A | A |
| A44 | B | A | B | B |
| A45 | C | B | D | C |
| A46 | B | A | B | B |
| A47 | B | B | C | D |
| A48 | C | A | C | C |
| A49 | B | A | B | B |
| A50 | B | A | B | B |
| A51 | B | A | B | A |
| A52 | B | A | B | B |
| A53 | A | A | A | A |
| A54 | B | A | B | A |
| A55 | B | A | B | A |
| A56 | B | A | B | A |
| A57 | C | B | C | D |
| A58 | B | A | C | B |
| A59 | B | A | C | B |
| A60 | B | A | B | A |
| A61 | B | A | B | B |
| A62 | B | A | B | B |
| A63 | B | A | B | A |
| A64 | B | A | C | B |
| A65 | C | C | | |
| A66 | B | A | B | B |
| A67 | C | A | C | A |
| A68 | C | B | C | B |
| A69 | C | B | D | D |
| A70 | C | B | C | C |
| A71 | B | A | C | B |
| A72 | B | A | C | B |
| A73 | C | B | C | C |
| A74 | C | C | | |
| A75 | B | A | C | C |
| A76 | C | D | | |
| A77 | C | B | C | D |
| A78 | C | A | C | C |
| A79 | C | B | C | B |
| A80 | D | B | C | D |
| A81 | C | A | C | B |
| A82 | C | B | C | D |
| A83 | B | A | B | A |
| A84 | E | E | E | E |
| A85 | B | B | C | D |
| A86 | B | A | C | B |
| A87 | C | A | C | D |
| A88 | B | C | C | C |
| A89 | C | C | C | D |
| A90 | B | A | C | B |
| A91 | B | A | C | C |
| A92 | C | A | C | B |
| A93 | D | B | C | C |
| A94 | C | A | C | B |
| A95 | D | B | D | C |
| A96 | C | A | C | B |
| A97 | C | B | D | D |
| A98 | B | B | D | D |
| A99 | C | A | C | B |
| A100 | D | C | D | D |
| A101 | B | A | B | B |
| A102 | B | B | C | D |
| A103 | B | A | C | B |
| A104 | D | C | | |
| A105 | A | A | B | C |
| A106 | E | D | | |
| A107 | D | B | | |
| A118 | C | D | | |
| A109 | B | A | B | A |
| A110 | A | A | A | A |
| A111 | A | A | A | A |
| A112 | E | E | | |
| A113 | A | A | A | B |
| A114 | D | B | | |
| A115 | D | D | | |
| A116 | C | A | C | B |
| A117 | B | A | A | A |
| A118 | C | D | | |
| A119 | B | A | A | A |
| A120 | C | A | C | A |
| A121 | A | A | B | A |
| A122 | A | A | A | A |
| A123 | B | A | B | A |
| A124 | A | A | A | A |
| A125 | B | A | B | A |
| | 6 h | | 24 h | |
| A126 | B | A | B | A |
| A127 | C | C | C | D |
| A128 | B | A | A | A |
| A129 | D | D | E | C |
| A130 | A | A | A | A |
| A131 | B | A | C | A |
| A132 | B | A | B | A |
| A133 | A | A | A | A |
| A134 | B | B | B | B |
| A135 | E | | | |
| A136 | D | D | | |
| A137 | B | C | C | B |
| A138 | B | B | C | B |
| A139 | B | A | C | A |
| A140 | B | B | B | B |
| A141 | B | A | B | B |
| A142 | B | A | B | A |
| A143 | B | A | B | A |
| A144 | C | A | D | B |
| A145 | A | A | B | A |
| A146 | B | A | B | A |
| A147 | B | A | B | A |
| A148 | D | D | | |
| A149 | C | D | | |
| A150 | C | B | C | B |
| A151 | B | B | B | B |
| A152 | D | D | | |
| A153 | B | A | B | A |
| A154 | B | A | B | A |
| A155 | C | D | D | D |
| A156 | B | A | B | A |
| A157 | B | A | B | B |
| A158 | B | B | B | B |
| A159 | B | A | B | A |
| A160 | B | A | B | A |
| A161 | B | B | B | B |
| A162 | C | A | C | B |
| A163 | C | C | | |
| A164 | C | B | | |
| A165 | C | B | C | B |
| A166 | B | A | C | A |
| A167 | B | A | B | A |
| A168 | B | A | B | A |
| A169 | B | A | B | A |
| A170 | B | A | B | B |
| A171 | B | A | C | B |
| A172 | C | B | B | D |
| A173 | B | A | B | A |
| A174 | B | A | C | B |
| A175 | B | A | B | A |
| A176 | B | B | C | B |
| A177 | C | C | | |
| A178 | C | A | C | A |
| A179 | C | C | | |
| A180 | B | B | C | B |
| A181 | B | A | B | A |
| A182 | C | B | | |
| A183 | C | B | C | B |
| A184 | B | A | B | A |
| A185 | C | D | D | D |
| A186 | B | A | B | A |
| A187 | B | A | B | A |
| A188 | B | A | B | A |
| A189 | B | A | B | A |
| A190 | B | A | C | B |
| A192 | B | A | C | A |
| A193 | B | A | C | B |
| A194 | B | D | B | B |
| A196 | B | B | C | B |
| A198 | B | B | C | B |
| A199 | B | A | B | A |
| A200 | B | A | C | B |
| A201 | B | A | C | B |
| A202 | B | A | B | A |
| A203 | B | A | C | A |
| A204 | C | A | C | A |
| A206 | B | A | C | B |
| A207 | B | A | C | B |
| A208 | B | A | B | A |
| A209 | B | A | B | A |
| A210 | D | C | | |
| A211 | E | E | E | E |
| A213 | D | D | | |
| A214 | B | A | C | B |
| A215 | C | B | D | B |
| A216 | C | B | D | C |
| A217 | E | E | | |
| A218 | E | E | E | E |
| A219 | D | C | E | E |
| A220 | B | B | C | C |
| A221 | C | C | C | C |
| A222 | E | E | E | D |
| A223 | D | D | E | E |
| A224 | D | D | E | D |
| A225 | B | B | C | C |
| A226 | C | B | D | D |
| A227 | B | C | C | D |
| A228 | C | C | D | D |
| A229 | C | B | C | C |
| A230 | C | C | | |
| A231 | C | C | D | D |
| A233 | E | E | | |
| A234 | B | B | C | B |
| A235 | C | A | C | B |
| A236 | E | E | | |
| A237 | B | B | C | B |
| A239 | D | C | | |
| A240 | C | B | D | C |
| A241 | C | B | C | B |
| A242 | B | A | B | A |
| A243 | C | B | D | D |
| A244 | C | B | C | C |
| A245 | C | B | | |
| A248 | B | A | B | B |
| A249 | C | A | C | A |
| A250 | C | B | C | C |
| A251 | E | E | | |
| A252 | C | C | | |
| A254 | B | B | B | B |
| A255 | B | A | B | B |
| A256 | B | A | B | B |
| A257 | B | A | C | B |
| A259 | B | A | C | B |
| A260 | B | A | C | B |
| A262 | B | A | C | B |
| A263 | C | D | | |
| A264 | C | D | | |
| A265 | B | A | C | B |
| A266 | E | E | | |
| A267 | C | B | C | B |
| A268 | C | B | D | D |
| A269 | C | B | C | C |
| A270 | C | A | C | B |
| A271 | E | E | | |
| A272 | B | A | B | A |
| A274 | C | A | D | A |
| A275 | D | C | | |
| A276 | D | D | | |
| A278 | C | B | | |
| A279 | D | D | E | C |
| A280 | B | B | C | B |
| A281 | D | C | | |
| A282 | B | A | C | B |
| A283 | B | A | C | B |
| A284 | C | A | C | B |
| A285 | C | B | D | B |
| A286 | C | D | | |
| A287 | B | A | C | B |
| A288 | B | A | C | A |
| A289 | B | A | C | A |
| A290 | E | E | E | E |
| A291 | C | B | | |
| A292 | D | D | | |
| A293 | D | D | | |
| A294 | C | C | | |
| A295 | B | A | C | B |
| A297 | E | E | E | E |
| A298 | C | B | | |
| A299 | B | B | C | B |
| A301 | C | C | | |
| A303 | B | A | C | A |
| A304 | B | A | C | B |
| A305 | D | C | D | D |
| A306 | B | A | C | B |
| A307 | B | A | C | B |
| A308 | E | E | D | D |
| A309 | C | C | D | C |
| A310 | C | C | D | C |
| A311 | B | B | C | C |
| A312 | C | A | C | B |
| A313 | B | A | C | B |
| A314 | B | A | C | B |
| A315 | C | C | C | D |
| A316 | C | D | E | D |
| A318 | C | B | C | B |
| A319 | C | D | C | C |
| A321 | C | A | C | B |
| A323 | D | D | E | D |
| A324 | C | A | C | B |
| A325 | B | B | C | C |
| A326 | C | A | C | A |
| A327 | B | A | C | B |
| A328 | B | A | B | A |
| A329 | E | E | E | E |
| A330 | E | E | E | E |
| A331 | D | D | D | C |
| A334 | D | D | D | C |
| A335 | B | A | B | A |
| A336 | B | A | C | B |
| A337 | C | D | C | B |
| A338 | C | C | C | B |
| A339 | C | D | D | D |
| A341 | C | C | E | F |
| A342 | C | C | D | 0 |
| A343 | B | A | C | B |
| A344 | C | C | D | D |
| A345 | E | E | E | E |
| A346 | B | B | C | C |
| A347 | B | A | C | B |
| A350 | C | A | C | B |
| A352 | B | A | C | B |
| A353 | C | B | C | C |
| A354 | C | B | D | D |
| A355 | B | A | D | D |
| A357 | B | B | C | D |
| A358 | E | D | E | C |
| A359 | B | D | E | E |
| A360 | E | E | E | E |
| A363 | B | A | B | B |
| A364 | B | A | B | A |
| A365 | C | C | D | D |
| A366 | B | A | B | A |
| A367 | C | C | D | D |
| A368 | B | C | C | D |
| A369 | C | B | C | C |
| A370 | C | C | B | D |
| A371 | C | C | C | D |
| A373 | B | A | B | A |
| A374 | B | A | C | B |
| A375 | E | E | E | E |
| A376 | C | B | C | C |
| A378 | B | A | C | B |
| A379 | B | A | C | B |
| A380 | C | A | C | C |
| A383 | C | D | D | D |
| A384 | B | B | B | C |
| A385 | B | A | B | B |
| A386 | C | B | C | C |
| A387 | B | A | C | B |
| A389 | C | B | C | C |
| A390 | B | A | C | B |
| A391 | B | A | B | A |
| A392 | B | A | B | A |
| A393 | B | A | B | A |
| A394 | C | A | C | B |
| A395 | B | A | C | B |
| A396 | B | A | B | A |
| A397 | B | A | B | A |
| A398 | C | B | C | C |
| A399 | A | A | A | A |
| A400 | D | D | | |
| A401 | B | B | D | B |
| A402 | C | B | C | C |
| A403 | C | B | D | D |

Definitions:
DC₅₀: “A”: < 1 nM; “B”: 1-9.9 nM; “C”: 10-99 nM; “D”: 100-999 nM; “E”: >1000 nM
D_{Max}: “A”: > 80%; “B”: 60-79%; “C”: 40-59%; “D”: 20-39%; “E”: < 20%.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula I
T-L-V (I′),
or a pharmaceutically acceptable salt, solvate, or stereoisomer thereof, wherein:
T is of Formula I′-1 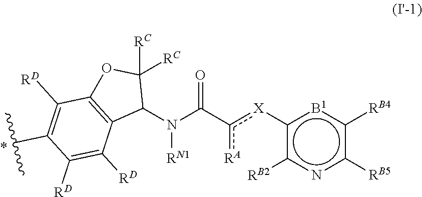
wherein:
* denotes attachment to L;
each [symbol] is independently a single bond or a double bond, as valency permits;
X is N(R^{X1}), and R^{A} is oxo; or
X is C(R^{X2}), and R^{A} and R^{B2}, together with the intervening atoms to which they are attached, form C₆ aryl or 6-membered heteroaryl, wherein the Co aryl or 6-membered heteroaryl is optionally substituted with one or more halogen, —CN, —NO₂, —OH, or —NH₂;
R^{X1} is hydrogen or C₁₋₆ alkyl, wherein the C₁₋₆ alkyl is optionally substituted with one or more halogen, —CN, —NO₂, —OH, or —NH₂;
R^{X2} is hydrogen, halogen, —CN, —NO₂, —OH, —NH₂, or C₁₋₆ alkyl, wherein the C₁₋₆ alkyl is optionally substituted with one or more halogen, —CN, —NO₂, —OH, or —NH₂;
B¹ is N, CR^{B1}, or NR^{BN1}, as valency permits;
R^{B1}, R^{B2}, R^{B4}, and R^{B5} are independently hydrogen, halogen, —CN, —NO₂, —OH, —NH₂, —C(═O)NH₂, —C(═O)(C₁₋₆ alkylamino), C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH, —NH₂, or C₁₋₆ alkyl; or
R^{B1} and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, or 3- to 12-membered heterocyclyl; or
R^{BN1} and R^{B4}, together with the intervening atoms to which they are attached, form 5-membered heteroaryl optionally substituted with one or more halogen, —CN, —NO₂, —OH, —NH₂, C1-6 alkyl, C₁₋₆ alkoxy, C₁₋₆ alkylamino, C₂₋₆ alkenyl, C₂₋₆ alkynyl, C₃₋₁₂ carbocyclyl, or 3- to 12-membered heterocyclyl;
R^{N1} is hydrogen, C₁₋₆ alkyl, or C₃₋₁₂ carbocyclyl, wherein the C₁₋₆ alkyl or C₃₋₁₂ carbocyclyl is optionally substituted with one or more halogen, —CN, —NO₂, —OH, —NH₂, or C₃₋₁₂ carbocyclyl;
each R^{C} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, or —NH₂; and
each R^{D} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN,-NO₂, —OH, or —NH₂;
L is of Formula I′-2 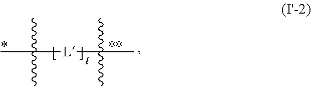
wherein:
* denotes attachment to T, and ** denotes attachment to V;
each L′ is independently —C(═O)—, —C(═O)N(R^{L′})—, —N(R^{L′})C(═O)—, —C(═O)O—, —OC(═O)—, —N(R^{L′})—,-O—,-O—(C₁₋₆ alkylene)-,-(C₁₋₆ alkylene)-O—, —S—, —S(═O)₂—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene, wherein the —O-(C₁₋₆ alkylene)-,-(C₁₋₆ alkylene)-O—, C₁₋₆ alkylene, C₁₋₆ heteroalkylene, C₂₋₆ alkenylene, C₂₋₆ alkynylene, C₃₋₁₂ carbocyclylene, 3- to 12-membered heterocyclylene, C₆₋₁₀ arylene, or 5- to 10-membered heteroarylene is optionally substituted with one or more oxo, halogen, —CN, —NO₂, —OH, —NH₂, or C₁₋₆ alkyl;
each occurrence of R^{L′} is independently hydrogen, C₁₋₆ alkyl, C₂₋₆ alkenyl, C₂₋₆ alkynyl, C₃₋₈ carbocyclyl, 3- to 8-membered heterocyclyl, —S(═O)₂R^{a}, —S(═O)₂OR^{a}, —S(═O)₂N(R^{a})₂, —C(═O)R^{a}, —C(═O)OR^{a}, or —C(═O)N(R^{a})₂, wherein the C₁₋₆ alkyl, C₂₋₆ alkenyl, C₂₋₆ alkynyl, C₃₋₈ carbocyclyl, or 3- to 8-membered heterocyclyl is optionally substituted with one or more halogen, —CN, —NO₂, —OH, or —NH₂; and
each R^{a} independently is hydrogen, C₁₋₆ alkyl, C₂₋₆ alkenyl, or C₂₋₆ alkynyl, wherein the C₁₋₆ alkyl, C₂₋₆ alkenyl, or C₂₋₆ alkynyl is optionally substituted with one or more halogen, —CN, —NO₂, —OH, or —NH₂;
1 is an integer selected from 0 to 10;
V is of Formula I′-3 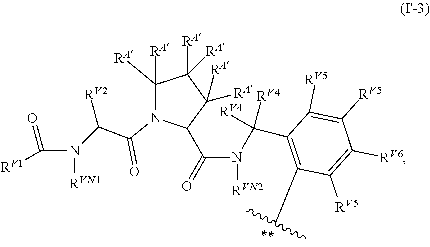
wherein:
** denotes attachment to L;
R^{V1} is hydrogen, C₁₋₆ alkyl, C₃₋₁₂ carbocyclyl, or 3- to 6-membered heterocyclyl, wherein the C₁₋₆ alkyl, C₃₋₆ carbocyclyl, or 3- to 6-membered heterocyclyl is optionally substituted with one or more halogen, —CN, —NO₂, —OH, —NH₂, or C₁₋₆ alkyl optionally substituted with one or more halogen;
R^{VN1} is hydrogen or C₁₋₆ alkyl, wherein the C₁₋₆ alkyl is optionally substituted with one or more halogen, —CN, —NO₂, —OH, or —NH₂;
R^{V2} is hydrogen, C₁₋₆ alkyl, C₃₋₆ carbocyclyl, 3- to 6-membered heterocyclyl, C₆₋₁₀ aryl, or 5- to 10-membered heteroaryl, wherein the C₁₋₆ alkyl, C₃₋₆ carbocyclyl, 3- to 6-membered heterocyclyl, C₆₋₁₀ aryl, or 5- to 10-membered heteroaryl is optionally substituted with one or more halogen, —CN, —NO₂, —OH, —NH₂, or C₁₋₆ alkyl;
each R^{A′} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN,-NO₂, —OH, or —NH₂;
R^{VN2} is hydrogen or C₁₋₆ alkyl, wherein the C₁₋₆ alkyl is optionally substituted with one or more halogen, —CN, —NO₂, —OH, or —NH₂;
each R^{V4} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN, —NO₂, —OH, —NH₂, C₃₋₁₂ carbocyclyl, 3- to 12-membered heterocyclyl, C₆₋₁₀ aryl, or 5- to 10-membered heteroaryl;
each R^{V5} is independently hydrogen, halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl, wherein the C₁₋₆ alkyl, C₁₋₆ alkoxy, C₁₋₆ alkylamino, C₂₋₆ alkenyl, or C₂₋₆ alkynyl is optionally substituted with one or more halogen, —CN,-NO₂, —OH, or —NH₂; and
R^{V6} is hydrogen, halogen, —CN, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl is optionally substituted with one or more halogen, —CN, —NO₂, —OH,-NH₂, C₁₋₆ alkyl optionally substituted with one or more-CN, C₂₋₆ alkenyl, or C₂₋₆ alkynyl.

2. The compound of claim 1, wherein X is N(R^{X1}), and R^{A} is oxo.

3. The compound of claim 1, wherein X is C(R^{X2}), and R^{A} and R^{B2}, together with the intervening atoms to which they are attached, form Ce aryl or 6-membered heteroaryl, wherein the C6 aryl or 6-membered heteroaryl is optionally substituted with one or more halogen, —CN, —NO₂, —OH, or —NH₂.

4. The compound of claim 3, wherein R^{A} and R^{B2}, together with the intervening atoms to which they are attached, form Co aryl or pyridinyl.

5. The compound of any one of claims 1-4, wherein RBI and R^{B4}, together with the intervening atoms to which they are attached, form 3- to 8-membered heterocyclyl or 5- to 6-membered heteroaryl, wherein the 3- to 8-membered heterocyclyl or 5- to 6-membered heteroaryl is optionally substituted with one or more halogen, —CN, —NO₂, —OH, —NH₂, C₁₋₆ alkyl, C₁₋₆ alkoxy, C₁₋₆ alkylamino, C₂₋₆ alkenyl, or C₂₋₆ alkynyl.

6. The compound of claim 5, wherein R^{B1} and R^{B4}, together with the intervening atoms to which they are attached, form dihydrofuran or pyrazole optionally substituted with one or more halogen or C₁₋₆ alkyl.

7. The compound of any one of claims 1-4, wherein RBI is hydrogen or C₁₋₆ alkyl, and R^{B4} is halogen, —CN, C₁₋₆ alkyl optionally substituted with one or more-OH, C₂₋₆ alkynyl, C₃₋₁₂ carbocyclyl, or 3- to 12-membered heterocyclyl.

8. The compound of any one of claims 1-7, wherein R^{B5} is hydrogen or —NH₂.

9. The compound of any one of claims 1-8, wherein each R^{C} is independently hydrogen.

10. The compound of any one of claims 1-9, wherein R^{N1} is C₁₋₆ alkyl optionally substituted with one or more C₃₋₁₂ carbocyclyl.

11. The compound of any one of claims 1-9, wherein R^{N1} is C₃₋₁₂ carbocyclyl.

12. The compound of any one of claims 1-11, wherein each R^{D} is independently hydrogen.

13. The compound of any one of claims 1-12, wherein T is of Formula I-1-a or I-1-b 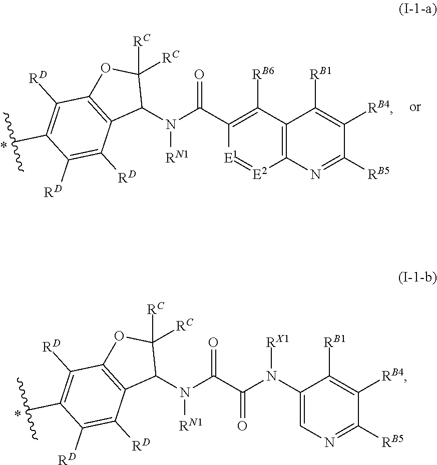
wherein:
E¹ is N or CR^{B7};
E² is N or CR^{B8}; and
R^{B6}, R^{B7}, and R^{B8} are independently hydrogen, halogen, —CN, —NO₂, —OH, or —NH₂.

14. The compound of any one of claims 1-13, wherein each L′ is independently selected from —C(═O)—, —C(═O)N(R^{L′})—, —N(R^{L′})C(═O)—, —N(R^{L′})—, —O—, —O—(C₁₋₆ alkylene)-,-(C₁₋₆ alkylene)-O—, C₁₋₆ alkylene, C₁₋₆ heteroalkylene, C₃₋₁₂ carbocyclylene, 3- to 12-membered heterocyclylene, and 5- to 10-membered heteroarylene.

15. The compound of any one of claims 1-14, wherein L is **-[O-(C₁₋₆ alkylene)]₁₋₈-N(R^{L′})C(═O)-(5- to 10-membered heteroarylene)-*, **-O-[(C₁₋₆ alkylene)]₁₋₃-N(R^{L′})C(═O)-(5- to 6-membered heteroarylene)-*, **-[O-(C₁₋₆ alkylene)]₁₋₇-(3- to 12-membered heterocyclylene)-C(═O)-(5- to 10-membered heteroarylene)-*, **-[O-(C1.6 alkylene)]₁₋₆—O—(C₃₋₁₂ carbocyclylene)-N(R^{L′})C(═O)-(5- to 10-membered heteroarylene)-*, or **-O-[(C₁₋₆ heteroalkylene)]₁₋₃-N(R^{L′})C(═O)-(5- to 10-membered heteroarylene)-*.

16. The compound of claim 15, wherein L is **-[O-(C₁₋₆ alkylene)]₁₋₈-N(H)C(═O)-(pyridinylene)-*, **-O-[(C₁₋₆ alkylene)]₁₋₃-N(H)C(═O)-(pyridinylene)-***-[O-(C₁₋₆ alkylene)]₁₋₇-(3- to 12-membered heterocyclylene)-C(═O)-(pyridinylene)-*, **-[O-(C₁₋₆ alkylene)]₁₋₆—O—(C₃₋₁₂ carbocyclylene)-N(H)C(═O)-(pyridinylene)-*, or **-O-[(C₁₋₆ heteroalkylene)]₁₋₃-N(H)C(═O)-(pyridinylene)-*.

17. The compound of any one of claims 1-16, wherein R^{V1} is C₁₋₆ alkyl or C₃₋₆ carbocyclyl, wherein the C₁₋₆ alkyl or C₃₋₆ carbocyclyl is optionally substituted with one or more halogen.

18. The compound of any one of claims 1-17, wherein R^{VN1} and R^{VN2} are each independently hydrogen.

19. The compound of any one of claims 1-18, wherein R^{V2} is C₁₋₆ alkyl.

20. The compound of any one of claims 1-19, wherein one RA′ is-OH, and each of the remaining R^{A′} is independently hydrogen.

21. The compound of any one of claims 1-20, wherein each R^{V4} is independently hydrogen.

22. The compound of any one of claims 1-21, wherein each R^{V5} is independently hydrogen.

23. The compound of any one of claims 1-22, wherein R^{V6} is C₂₋₆ alkynyl or 5- to 10-membered heteroaryl optionally substituted with one or more C₁₋₆ alkyl.

24. The compound of any one of claims 1-23, wherein Vis of Formula I-3-a

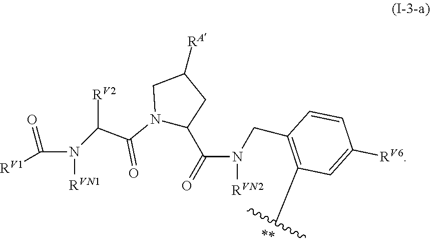

25. The compound of claim 1, wherein the compound is of Formula (I′-a) 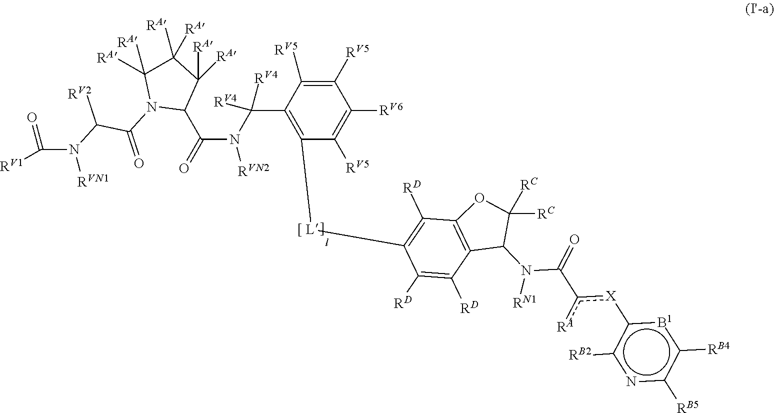
or a pharmaceutically acceptable salt, solvate, or stereoisomer thereof.

26. The compound of claim 1, wherein the compound is of Formula (I′-a-i), (I′-a-ii), or (I′-a-iii) 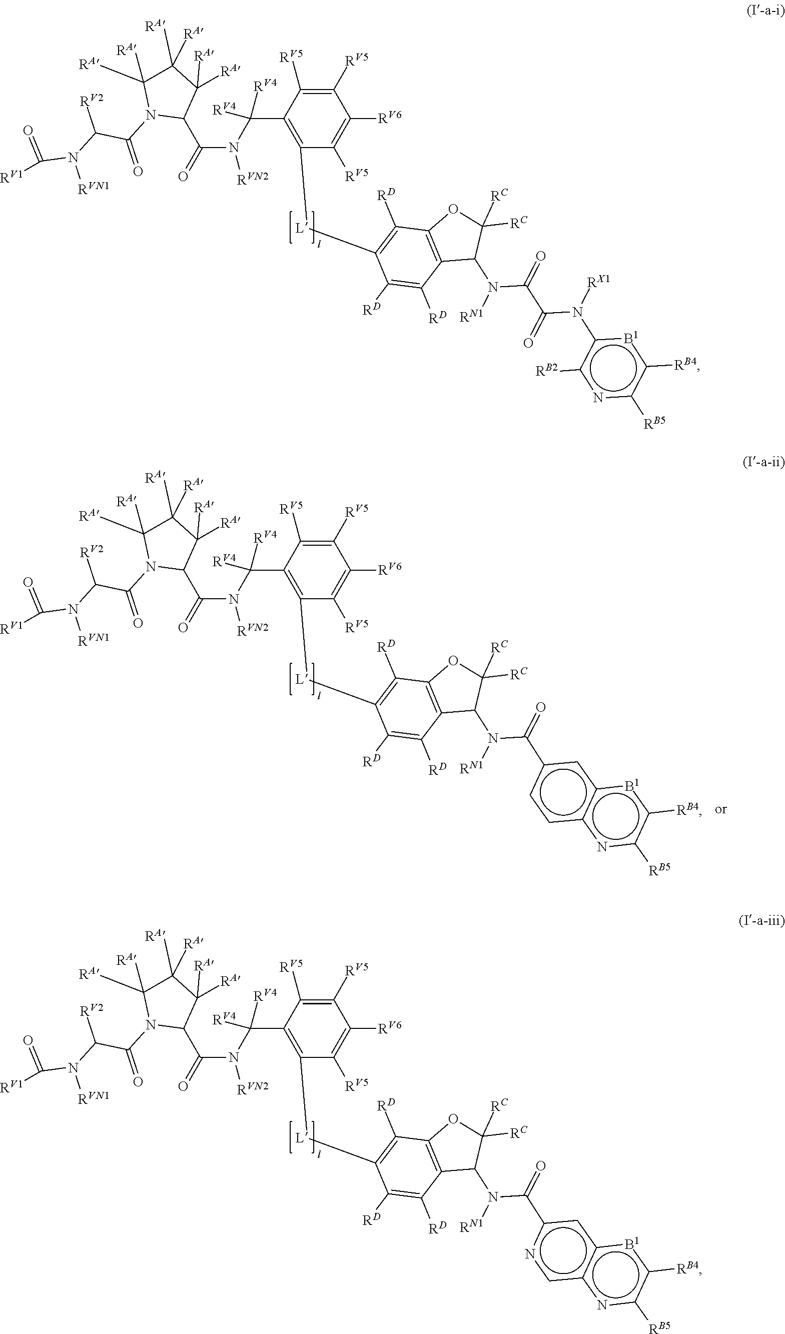
or a pharmaceutically acceptable salt, solvate, or stereoisomer thereof.

27. A compound selected from the compounds in Table 1, or a pharmaceutically acceptable salt, solvate, or stereoisomer thereof.

28. A pharmaceutical composition comprising the compound of any one of claims 1-27, and one or more pharmaceutically acceptable excipient.

29. A method of treating a disease or disorder comprising administering to a patient in need thereof a compound of any one of claims 1-27.

30. Use of a compound of any one of claims 1-27 in the manufacture of a medicament for treating a disease or disorder.

31. A compound of any one of claims 1-27 for use in treating a disease or disorder.

32. The method, use, or compound for use of any one of claims 29-31, wherein the disease or disorder is a PRMT5 protein-mediated disease or disorder.

33. The method, use, or compound for use of any one of claims 29-32, wherein the disease or disorder is cancer.

34. The compound of any one of claims 1-27, wherein the compound is conjugated with a conjugate partner.

35. A conjugate comprising the compound of any one of claims 1-27 and a conjugate partner.

36. The compound of claim 34 or the conjugate of claim 35, wherein the conjugate partner is an antibody.

37. The compound of claim 34 or the conjugate of claim 35, wherein the conjugate partner is a half-life extending moiety.

38. A method of preparing a compound according to any one of claims 1-27, or any intermediate in the Examples, according to a scheme of the present disclosure or a synthetic description in the Examples.

39. An intermediate described in one or more Examples.
